(19) United States
(12) Patent Application Publication
(10) Pub. No.: US 2025/0332173 A1
(43) Pub. Date: Oct. 30, 2025

(54) COMPOUNDS AND METHODS FOR MODULATING NUCLEIC ACID SPLICING

(71) Applicant: REMIX THERAPEUTICS INC., Watertown, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Peter Smith, Arlington, MA (US); Sudeep Prajapati, Somerville, MA (US); Allen T. Hopper, Lexington, MA (US); Stepan Vyskocil, Benesov (CZ); Benoît Moreau, Newton, MA (US)

(21) Appl. No.: 18/700,632

(22) PCT Filed: Oct. 13, 2022
(86) PCT No.: PCT/US2022/078081

§ 371 Date: Apr. 11, 2024

Related U.S. Application Data
(60) Provisional application No. 63/255,348, filed on Oct. 13, 2021.
(60) Provisional application No. 63/255,079, filed on Oct. 13, 2021.
(60) Provisional application No. 63/255,178, filed on Oct. 13, 2021.
(60) Provisional application No. 63/393,210, filed on Jul. 28, 2022.
(60) Provisional application No. 63/393,208, filed on Jul. 28, 2022.

(51) Int. Cl. A61K 31/55; A61K 31/4375; A61K 31/438; A61K 31/4545; A61K 31/4725; A61K 31/4985; A61K 31/5025; A61K 31/517; A61K 31/519; C07D 401/14; C07D 413/14; C07D 417/14; C07D 471/04; C07D 487/04; C07D 498/04; C07D 519/00

(52) U.S. Cl. CPC A61K 31/55; A61K 31/4375; A61K 31/438; A61K 31/4545; A61K 31/4725; A61K 31/4985; A61K 31/5025; A61K 31/517; A61K 31/519; C07D 401/14; C07D 413/14; C07D 417/14; C07D 471/04; C07D 487/04; C07D 498/04; C07D 519/00

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application is a U.S. National Phase Application under 35 U.S.C. § 371 of International Application No. PCT/US2022/078081, filed Oct. 13, 2022, which claims priority to U.S. Application No. 63/255,178, filed on Oct. 13, 2021; U.S. Application No. 63/255,348, filed on Oct. 13, 2021; U.S. Application No. 63/255,079, filed on Oct. 13, 2021; U.S. Application No. 63/393,208, filed on Jul. 28, 2022; and U.S. Application No. 63/393,210, filed on Jul. 28, 2022. The disclosure of each of the foregoing applications is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formulas (I), (II), (III), (IV), (V), (VI), (VII), (VIII), or (IX) and pharmaceutically acceptable salts, solvates, hydrates, tautomers, or stereoisomers thereof. The present disclosure additionally provides methods of using the compounds of the disclosure (e.g., compounds of Formulas (I), (II), (III), (IV), (V), (VI), (VII), (VIII), or (IX) and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I), (II), (III), (IV), (V), (VI), (VII), (VIII), or (IX) and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I), (II), (III), (IV), (V), (VI), (VII), (VIII), or (IX) and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I), (II), (III), (IV), (V), (VI), (VII), (VIII), or (IX), and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the present disclosure provides compounds of Formula (I):

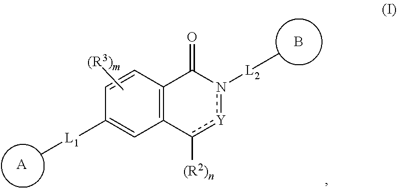

or a pharmaceutically acceptable salt, solvate, hydrate, tautomer, or stereoisomer thereof, wherein each of A, B, L¹, L², Y, R², R³, m, n, and subvariables thereof are defined as described herein.

In another aspect, the present disclosure features compounds of Formula (II):

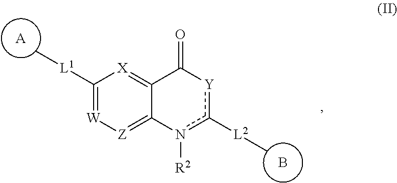

or a pharmaceutically acceptable salt, solvate, hydrate, tautomer, or stereoisomer thereof, wherein each of A, B, L¹, L², W, X, Y, Z, R², and subvariables thereof are defined as described herein.

In another aspect, the present disclosure provides compounds of Formula (III):

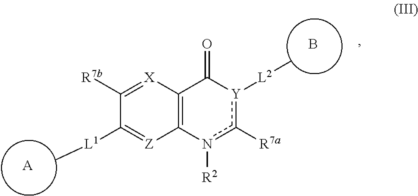

or a pharmaceutically acceptable salt, solvate, hydrate, tautomer, or stereoisomer thereof, wherein each of A, B, L¹, L², X, Y, Z, R², R^{7a}, R^{7b}, and subvariables thereof are defined as described herein.

In another aspect, the present disclosure provides compounds of Formula (IV):

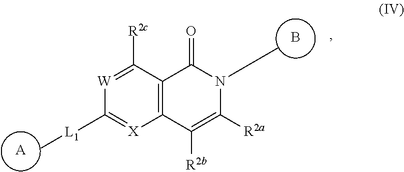

or a pharmaceutically acceptable salt, solvate, hydrate, tautomer, or stereoisomer thereof, wherein each of A, B, L¹, L², W, X, R^{2a}, R^{2b}, R^{2c}, and subvariables thereof are defined as described herein.

In another aspect, the present disclosure provides compounds of Formula (V):

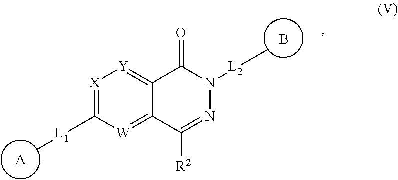

or a pharmaceutically acceptable salt, solvate, hydrate, tautomer, or stereoisomer thereof, wherein each of A, B, L¹, L², W, X, Y, R², and subvariables thereof are defined as described herein.

In another aspect, the present disclosure provides compounds of Formula (VI):

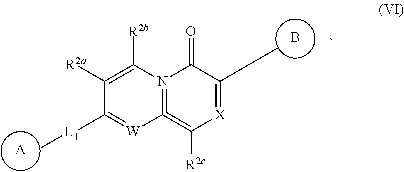

or a pharmaceutically acceptable salt, solvate, hydrate, tautomer, or stereoisomer thereof, wherein each of A, B, L¹, L², W, X, R^{2a}, R^{2b}, R^{2c}, and subvariables thereof are defined as described herein.

In another aspect, the present disclosure provides compounds of Formula (VII):

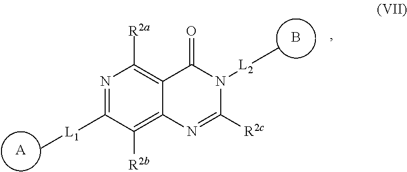

or a pharmaceutically acceptable salt, solvate, hydrate, tautomer, or stereoisomer thereof, wherein each of A, B, L¹, L², R^{2a}, R^{2b}, R^{2c}, and subvariables thereof are defined as described herein.

In another aspect, the present disclosure provides compounds of Formula (VIII):

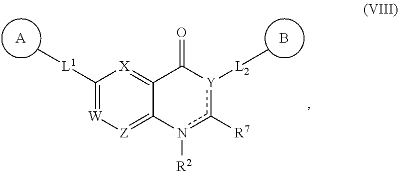

or a pharmaceutically acceptable salt, solvate, hydrate, tautomer, or stereoisomer thereof, wherein each of A, B, L¹, L², W, X, Y, Z, R², R⁷, and subvariables thereof are defined as described herein.

In another aspect, the present disclosure provides compounds of Formula (IX):

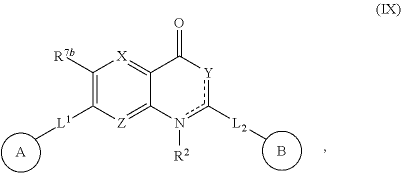

or a pharmaceutically acceptable salt, solvate, hydrate, tautomer, or stereoisomer thereof, wherein each of A, B, L, L², X, Y, Z, R², R^{7b}, and subvariables thereof are defined as described herein.

In another aspect, the present invention provides pharmaceutical compositions comprising a compound of Formulas (I), (II), (III), (IV), (V), (VI), (VII), (VIII), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formulas (I), (II), (III), (IV), (V), (VI), (VII), (VIII), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formulas (I), (II), (III), (IV), (V), (VI), (VII), (VIII), or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formulas (I), (II), (III), (IV), (V), (VI), (VII), (VIII), or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formulas (I), (II), (III), (IV), (V), (VI), (VII), (VIII), or (IX) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formulas (I), (II), (III), (IV), (V), (VI), (VII), (VIII), or (IX) alters a target nucleic acid (e.g., DNA or RNA, e.g., a precursor RNA, e.g., a pre-mRNA), target protein, or combination thereof. In some embodiments, the compound of Formulas (I), (II), (III), (IV), (V), (VI), (VII), (VIII), or (IX) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I), (II), (III), (IV), (V), (VI), (VII), (VIII), or (IX), e.g., in a healthy or diseased cell or tissue). In some embodiments, the presence of a compound of Formulas (I), (II), (III), (IV), (V), (VI), (VII), (VIII), or (IX) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I), (II), (III), (IV), (V), (VI), (VII), (VIII), or (IX), e.g., in a healthy or diseased cell or tissue).

In another aspect, the present disclosure provides methods for preventing and/or treating a disease, disorder, or condition in a subject by administering a compound of Formulas (I), (II), (III), (IV), (V), (VI), (VII), (VIII), or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formulas (I), (II), (III), (IV), (V), (VI), (VII), (VIII), or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formulas (I), (II), (III), (IV), (V), (VI), (VII), (VIII), or a pharmaceutically acceptable salt, solvate, hydrate, tautomer, or stereoisomer thereof in a biological sample or subject. In another aspect, the present disclosure provides methods of altering the isoform of a target protein with a compound of Formulas (I), (II), (III), (IV), (V), (VI), (VII), (VIII), or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formulas (I), (II), (III), (IV), (V), (VI), (VII), (VIII), or (IX) to a biological sample, a cell, or a subject comprises inhibition of cell growth or induction of cell death.

In another aspect, the present disclosure provides compositions for use in preventing and/or treating a disease, disorder, or condition in a subject by administering a compound of Formulas (I), (II), (III), (IV), (V), (VI), (VII), (VIII), or (IX) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides compositions for use in treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formulas (I), (II), (III), (IV), (V), (VI), (VII), (VIII), or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formulas (I), (II), (III), (IV), (V), (VI), (VII), (VIII), or a pharmaceutically acceptable salt, solvate, hydrate, tautomer, or stereoisomer thereof in a biological sample or subject. In another aspect, the present disclosure provides compositions for use in altering the isoform of a target protein with a compound of Formulas (I), (II), (III), (IV), (V), (VI), (VII), (VIII), or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formulas (I), (II), (III), (IV), (V), (VI), (VII), (VIII), or (IX) to a biological sample, a cell, or a subject comprises inhibition of cell growth or induction of cell death.

In another aspect, the present disclosure features kits comprising a container with a compound of Formulas (I), (II), (III), (IV), (V), (VI), (VII), (VIII), or a pharmaceutically acceptable salt, solvate, hydrate, tautomer, stereoisomer thereof, or a pharmaceutical composition thereof. In certain embodiments, the kits described herein further include instructions for administering the compound of Formulas (I), (II), (III), (IV), (V), (VI), (VII), (VIII), or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2020/004594. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2020/004594, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₅ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂-10 alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—Cl₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 16-membered non-aromatic ring system having ring carbon atoms and 1 to 4 ring heteroatoms, wherein each heteroatom is independently selected from nitrogen, oxygen, sulfur, boron, phosphorus, and silicon (“3-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′—and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substituent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereomerically pure exo compound can comprise, for example, about 90% excipient and about 10% diastereomerically pure exo compound. In certain embodiments, the diastereomerically pure exo compound in such compositions can, for example, comprise, at least about 95% by weight exo compound and at most about 5% by weight endo compound, by total weight of the compound. For example, a pharmaceutical composition comprising a diastereomerically pure endo compound can comprise, for example, about 90% excipient and about 10% diastereomerically pure endo compound. In certain embodiments, the diastereomerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formulas (I), (II), (III), (IV), (V), (VI), (VII), (VIII), or (IX) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomer” refers to compounds that are interchangeable forms of a particular compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formulas (I), (II), (III), (IV), (V), (VI), (VII), (VIII), or (IX) refers to an amount sufficient to elicit the desired biological response, i.e., treating the condition. As will be appreciated by those of ordinary skill in this art, the effective amount of a compound of Formulas (I), (II), (III), (IV), (V), (VI), (VII), (VIII), or (IX) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formulas (I), (II), (III), (IV), (V), (VI), (VII), (VIII), or (IX) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formulas (I), (II), (III), (IV), (V), (VI), (VII), (VIII), or (IX)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formulas (I), (II), (III), (IV), (V), (VI), (VII), (VIII), or (IX)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

In one aspect, the present disclosure features a compound of Formula (I):

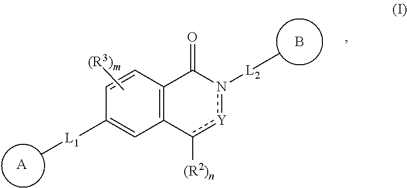

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; R^{B} is B, C₁-C₆-alkyl, or C₁-C₆-heteroalkyl, wherein alkyl and heteroalkyl are substituted by one or more R¹⁰; B is cycloalkyl, heterocyclyl, aryl, or heteroaryl, each of which is optionally substituted with one or more R¹; each of which is optionally substituted with one or more R¹; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁹; Y is N, C(R^{6a}), or C(R^{6a})(R^{6b}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, —NR^{B}C(O)R^{D}, or —S(O)ₓR^{D}; R³ is C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, C(O)NR^{B}R^{C}, —NR^{B}C(O)R^{D}, or —S(O)ₓR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; R^{6a} and R^{6b} is independently hydrogen, C₁-C₆-alkyl, C₁-C₆-heteroalkyl, C₁-C₆-haloalkyl, or halo; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆-alkenyl, C₂-C₆-alkynyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₂-C₆-alkenyl, C₂-C₆-alkynyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —OR^{A}, —S(O)ₓR^{D}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ and R¹⁰ is independently C₁-C₆-alkyl, halo, or —OR^{A1}; n is 0, 1, or 2; m is 0, 1, 2, or 3; and x is 0, 1, or 2.

In another aspect, the present disclosure features a compound of Formula (II):

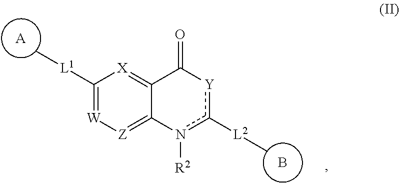

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently is absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of W, X, and Z is independently C(R³) or N; Y is N, N(R^{4a}), C(R^{4b}), or C(R^{4b})(R^{4c}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)R^{D}, or —S(O)ₓR^{D}; R^{4a} is hydrogen, C₁-C₆-alkyl, C₁-C₆-heteroalkyl, or C₁-C₆-haloalkyl; each of R^{4b} and R^{4c} is independently hydrogen, C₁-C₆-alkyl, C₁-C₆-heteroalkyl, C₁-C₆-haloalkyl, halo, or —OR^{A}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In another aspect, the present disclosure features a compound of Formula (III):

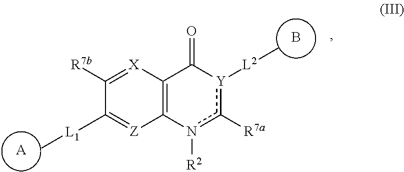

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of X and Z is independently C(R³) or N; Y is N, C, or C(R^{4b}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)R^{D}, or —S(O)ₓR^{D}; R^{4b} is hydrogen, C₁-C₆-alkyl, C₁-C₆-heteroalkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R^{7a} is hydrogen, C₁-C₆-alkyl, C₁-C₆-heteroalkyl, C₁-C₆-haloalkyl, halo, cyano, oxo, or —OR^{A}; R^{7b} is hydrogen, C₁-C₆-alkyl, C₁-C₆-heteroalkyl, C₁-C₆-haloalkyl, halo, cyano, or —OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₂-C₆-alkenyl, C₂-C₆-alkynyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₂-C₆-alkenyl, C₂-C₆-alkynyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —OR^{A}, —S(O)ₓR^{D}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In another aspect, the present disclosure features a compound of Formula (IV):

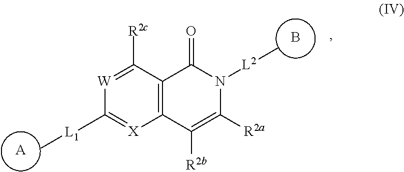

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W and X are each independently C(R³) or N; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁶; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R^{2a}, R^{2b}, and R^{2c} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; each R⁶ and R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —C(O)R^{D}, or —S(O)ₓR^{D}; each R^{B} and R^{C} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —OR^{A}, —S(O)ₓR^{D}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2 In another aspect, the present disclosure features a compound of Formula (V):

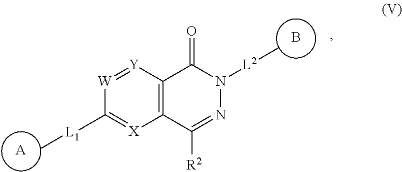

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W, X, and Y are each independently C(R³) or N, wherein at least one of W, X, and Y is independently N; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁶; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; each R⁶ and R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —C(O)R^{D}, or —S(O)ₓR^{D}; each R^{B} and R^{C} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —OR^{A}, —S(O)ₓR^{D}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In another aspect, the present disclosure features a compound of Formula (VI):

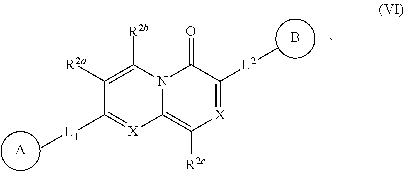

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W and X are each independently C(R³) or N; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁶; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R^{2a}, R^{2b}, and R^{2c} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; each R⁶ and R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —C(O)R^{D}, or —S(O)ₓR^{D}; each R^{B} and R^{C} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —OR^{A}, —S(O)ₓR^{D}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In another aspect, the present disclosure features a compound of Formula (VII):

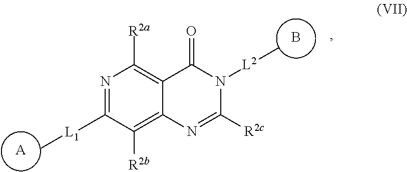

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁶; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R^{2a}, R^{2b}, and R^{2c} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; each R⁶ and R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —C(O)R^{D}, or —S(O)ₓR^{D}; each R^{B} and R^{C} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —OR^{A}, —S(O)ₓR^{D}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; and x is 0, 1, or 2.

In another aspect, the present invention features a compound of Formula (VIII):

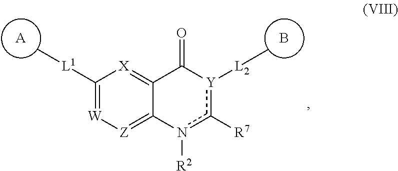

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of W, X, and Z is independently C(R³) or N; Y is N, C, or C(R^{4b}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R^{4b} is hydrogen, C₁-C₆-alkyl, C₁-C₆-heteroalkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R⁷ is hydrogen, C₁-C₆-alkyl, C₁-C₆-heteroalkyl, C₁-C₆-haloalkyl, halo, cyano, oxo, or —OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In another aspect, the present disclosure features a compound of Formula (IX):

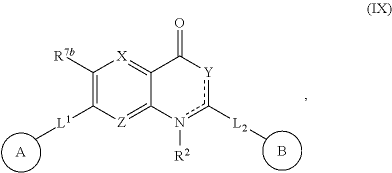

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of X and Z is independently C(R³) or N; Y is N, N(R^{4a}), C(R^{4b}), or C(R^{4b})(R^{4c}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R^{4a} is hydrogen, C₁-C₆-alkyl, C₁-C₆-heteroalkyl, or C₁-C₆-haloalkyl; each of R^{4b} and R^{4c} is independently hydrogen, C₁-C₆-alkyl, C₁-C₆-heteroalkyl, C₁-C₆-haloalkyl, halo, or —OR^{A}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R^{7b} is hydrogen, C₁-C₆-alkyl, C₁-C₆-heteroalkyl, C₁-C₆-haloalkyl, halo, cyano, or —OR^{A}; each R⁵ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, -NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

As generally described herein for compounds of Formula (I), (II), (III), (IV), (V), (VI), (VII), (VIII), and (IX), each of A or B are independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, B is a nitrogen-containing bicyclic heteroaryl (e.g., a 9-membered nitrogen-containing bicyclic heteroaryl), that is optionally substituted with one or more R¹. In some embodiments, B is a 9-membered bicyclic heteroaryl comprising 1 nitrogen atom. In some embodiments, B is a 9-membered bicyclic heteroaryl comprising 2 nitrogen atoms. In some embodiments, B is a 9-membered bicyclic heteroaryl comprising 3 nitrogen atoms. In some embodiments, B is a 9-membered bicyclic heteroaryl comprising 4 nitrogen atoms. The one or more nitrogen atom of the 9-membered bicyclic heteroaryl may be at any position of the ring. In some embodiments, B is a 9-membered bicyclic heteroaryl substituted with one or more R¹.

In some embodiments, each of A and B are independently selected from:

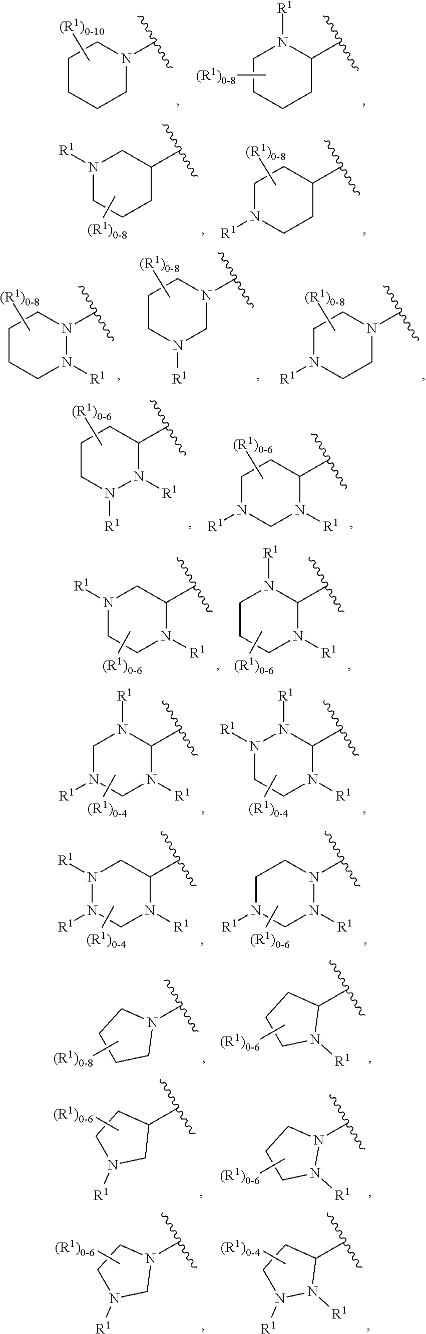

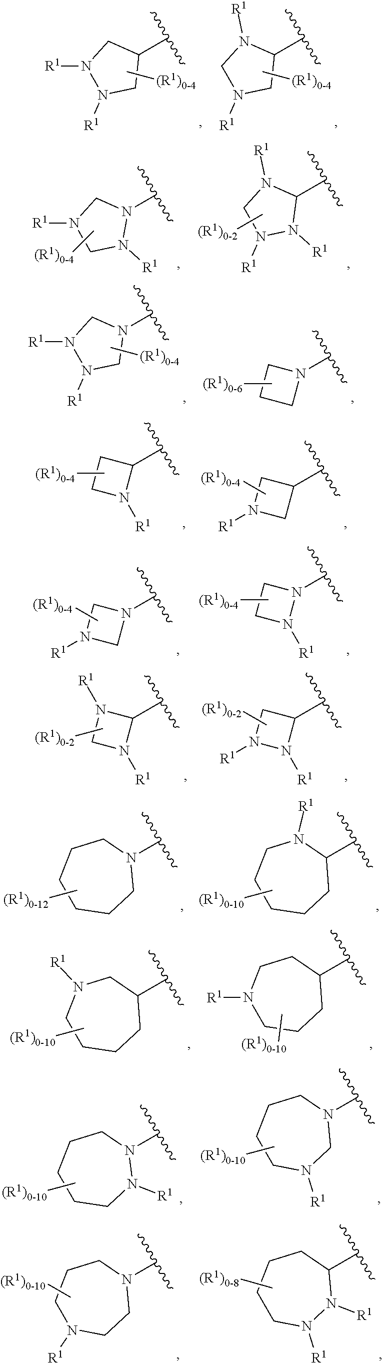

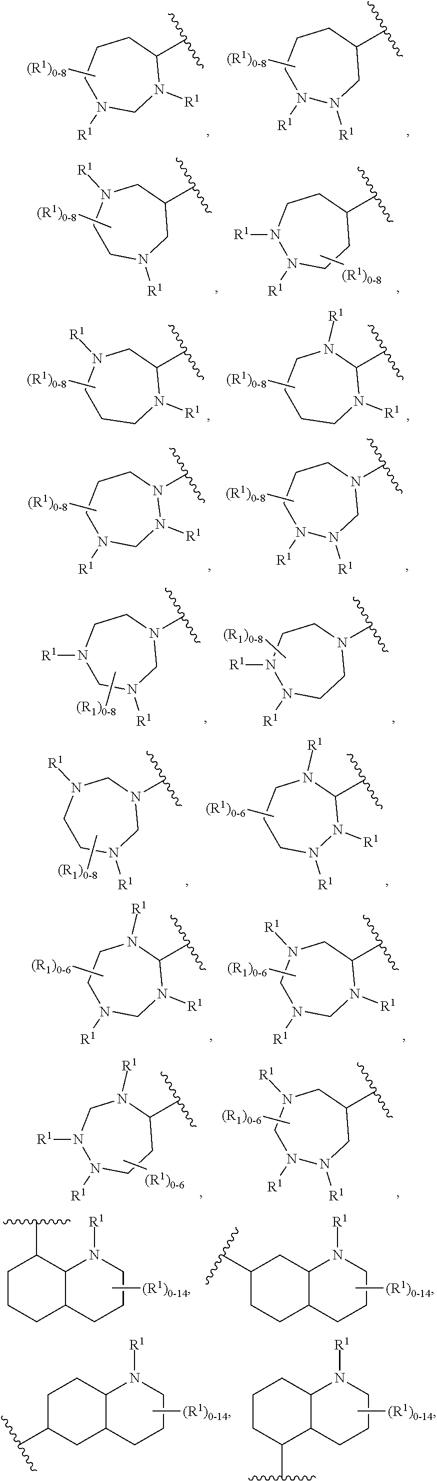

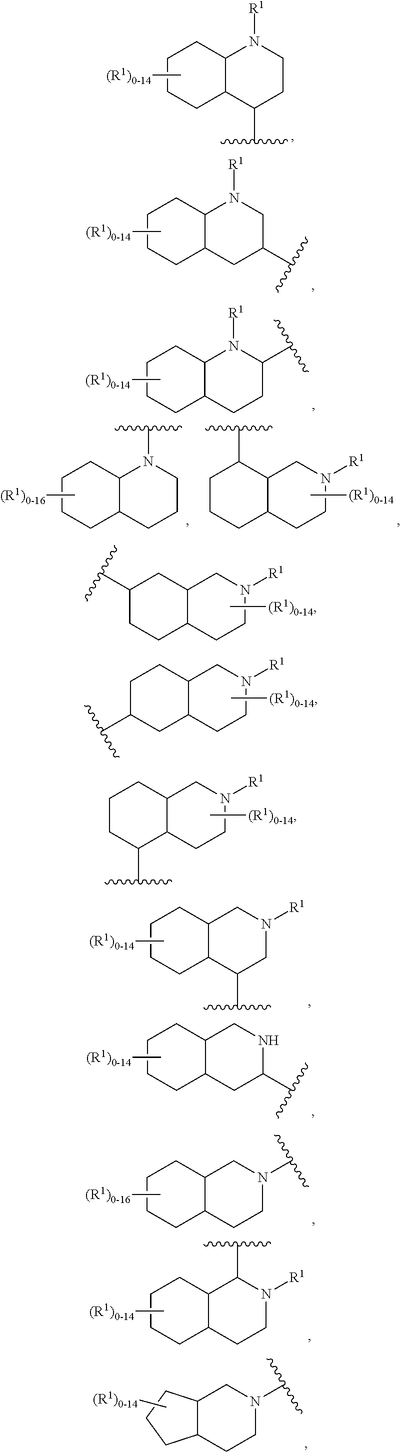

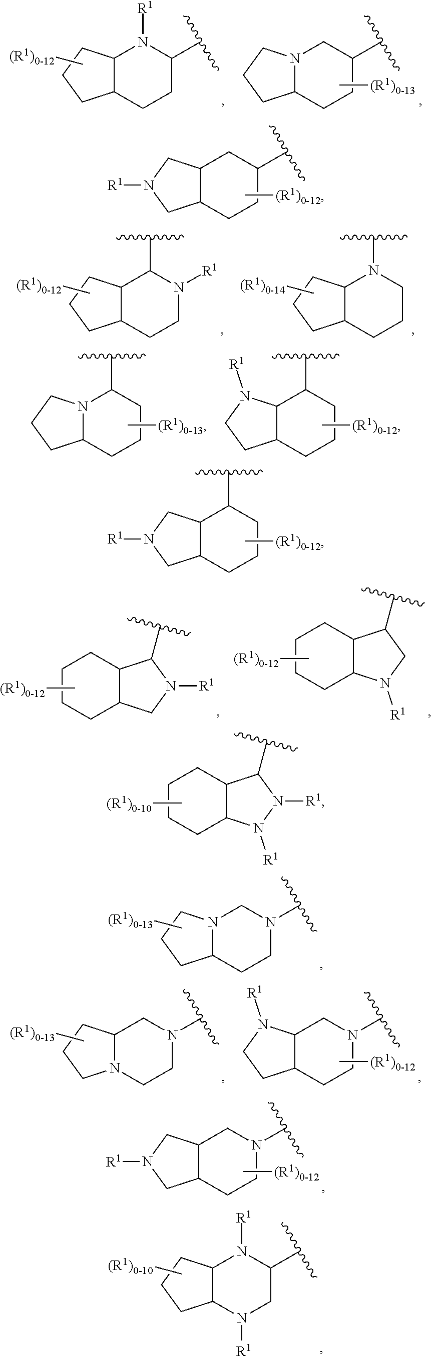

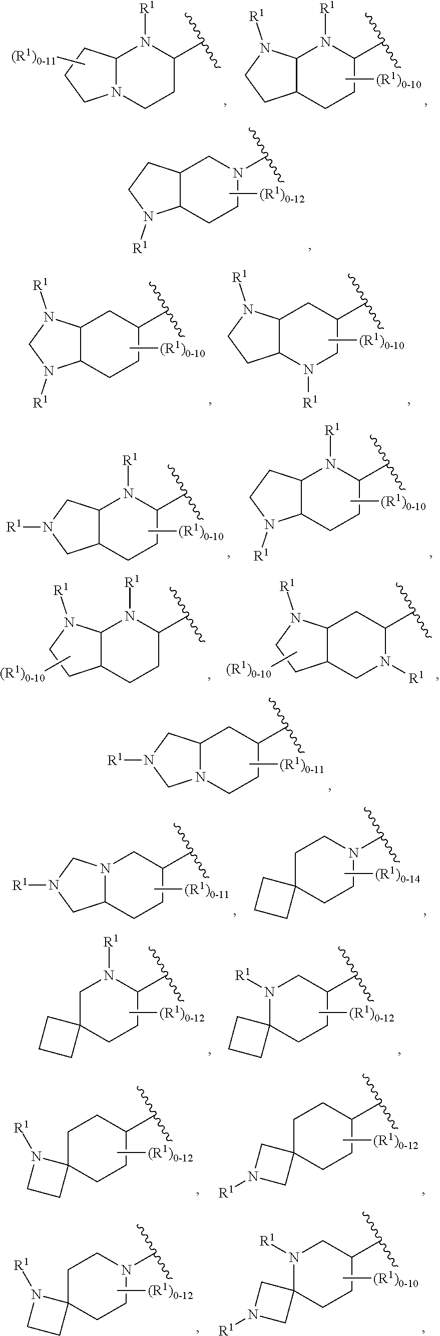

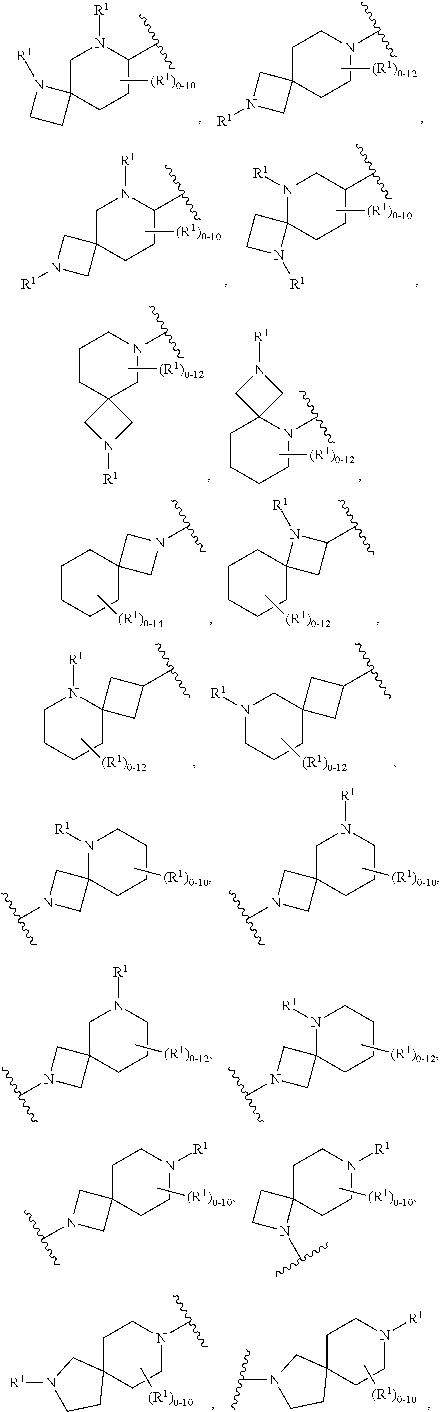

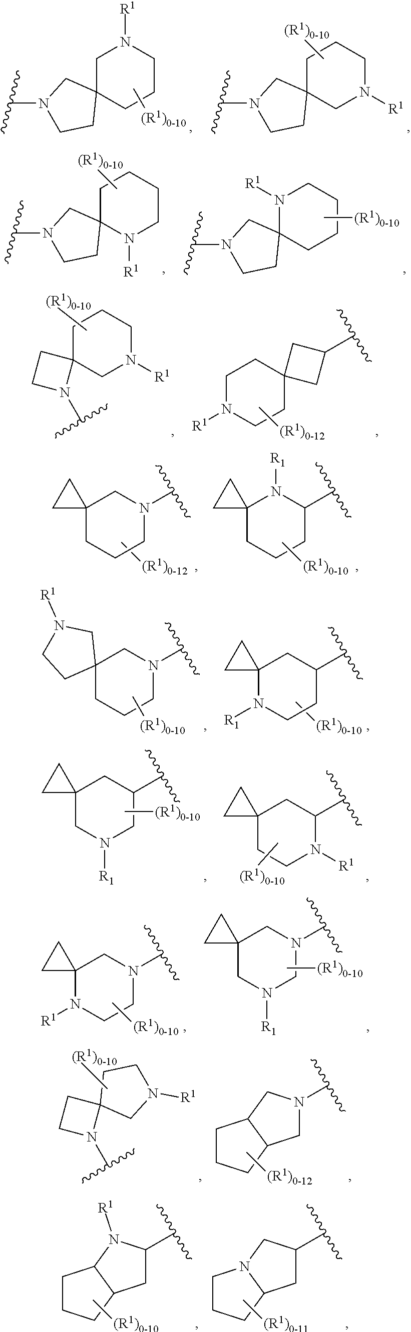

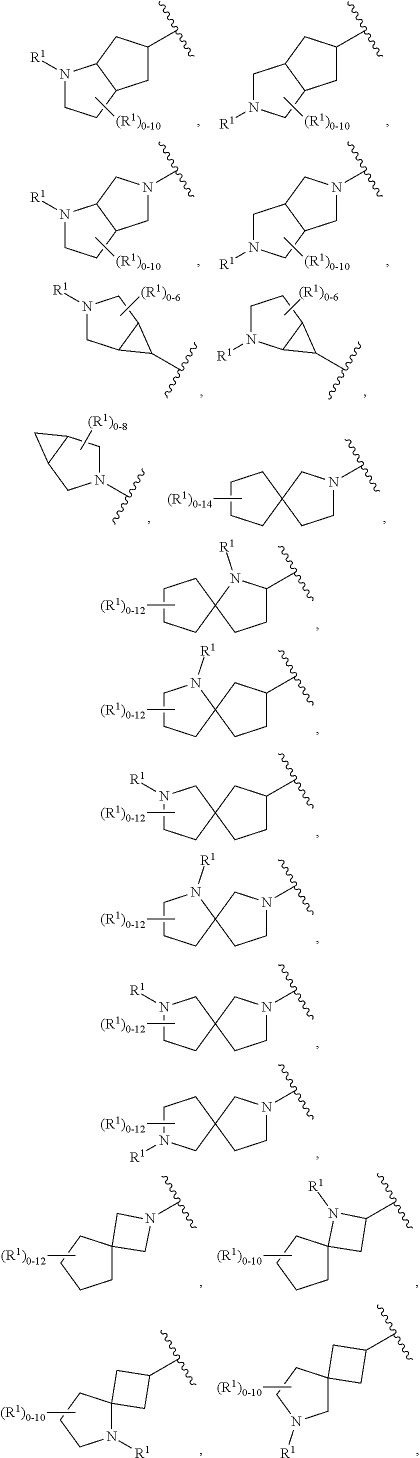

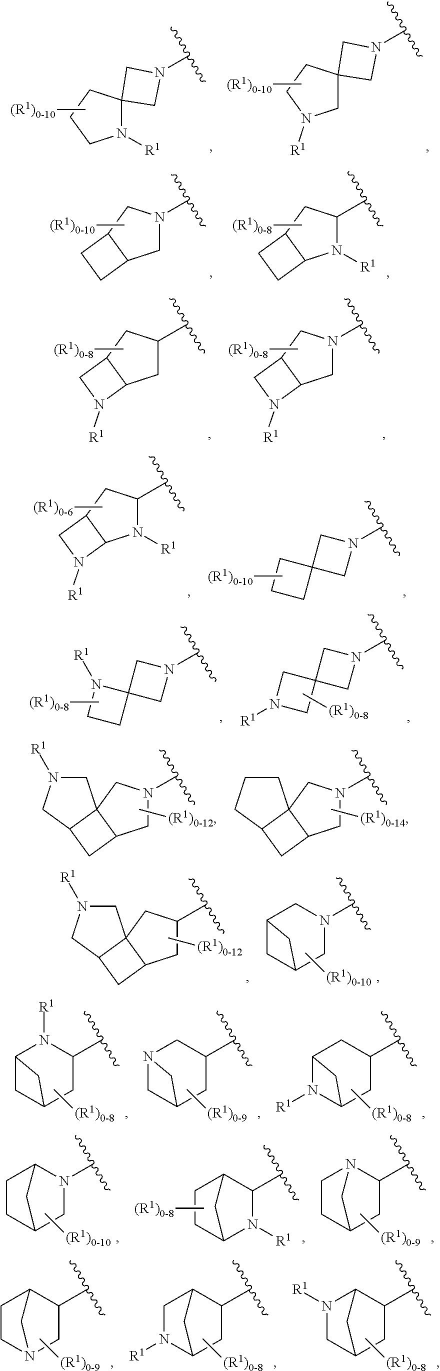

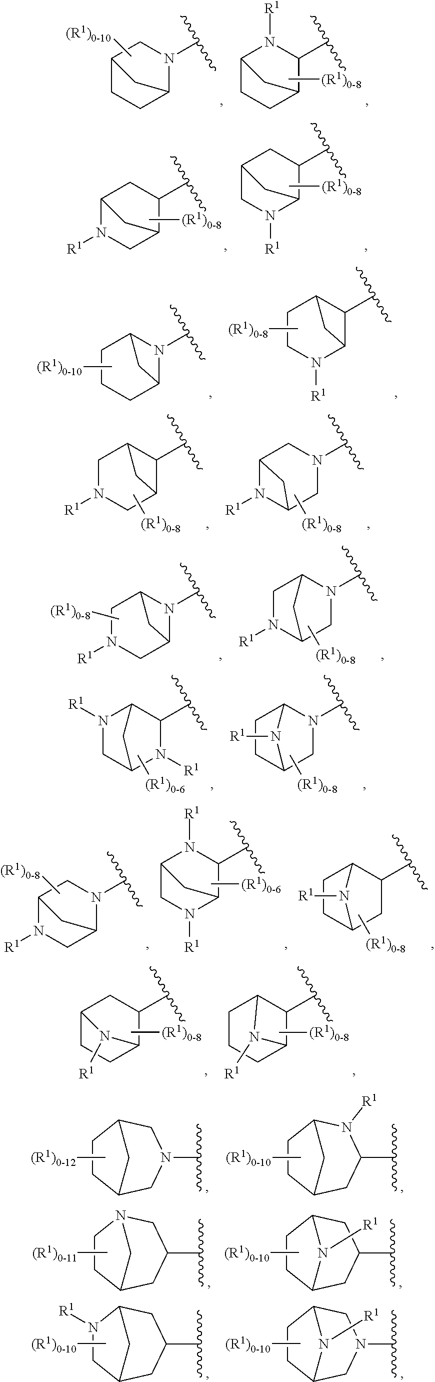

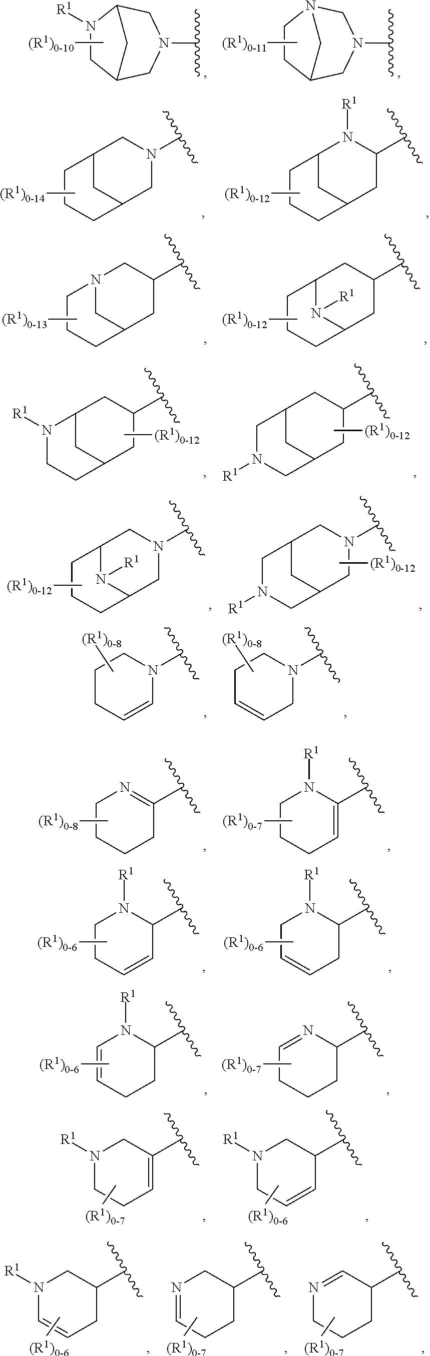

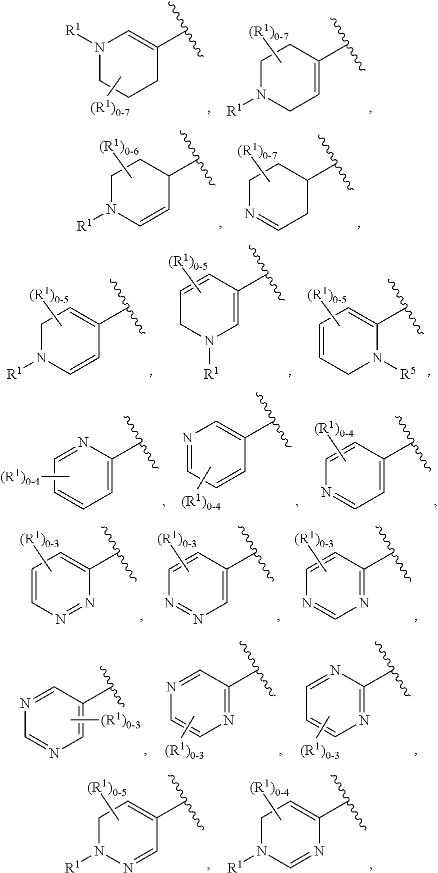

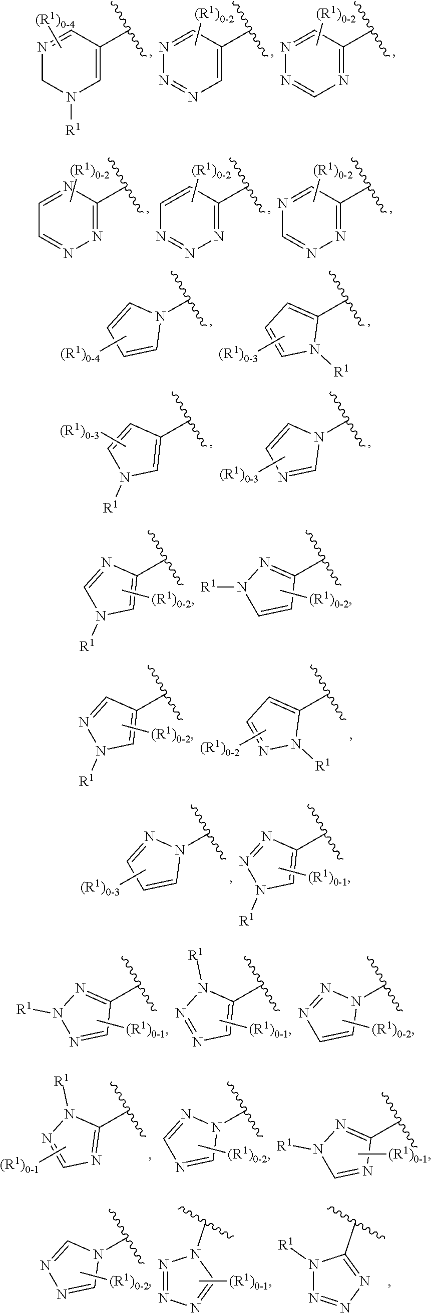

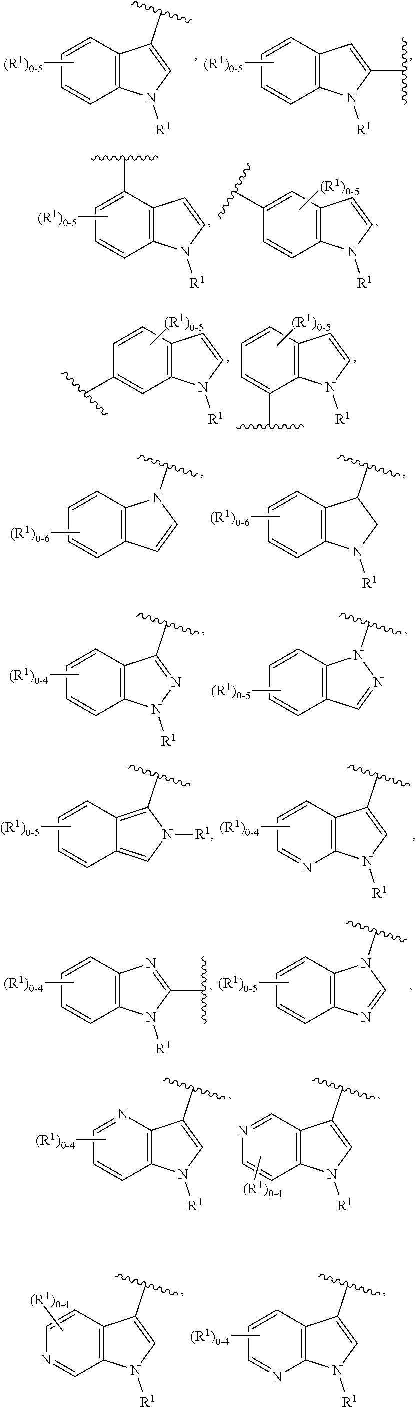

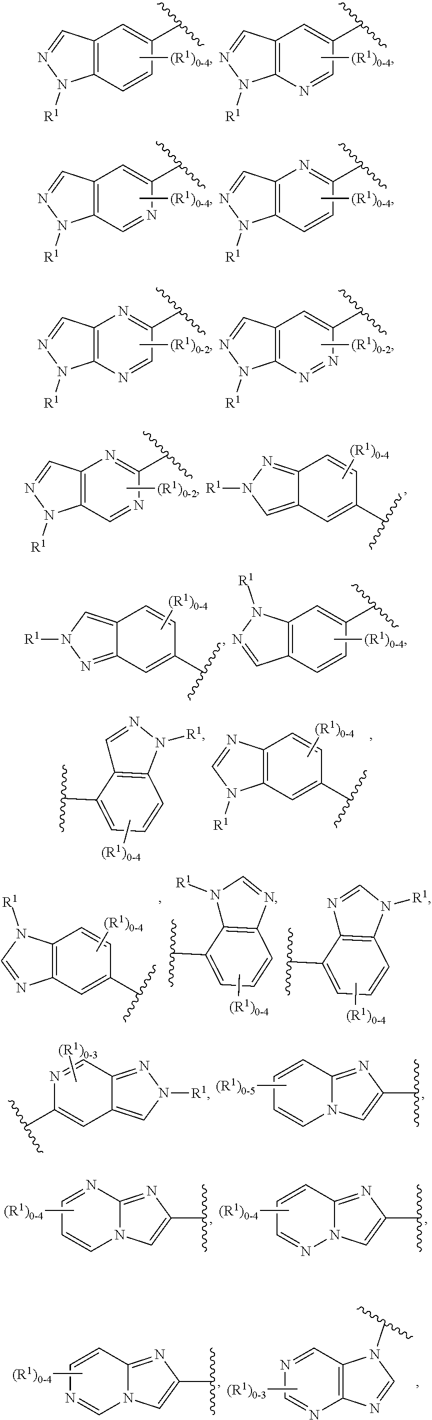

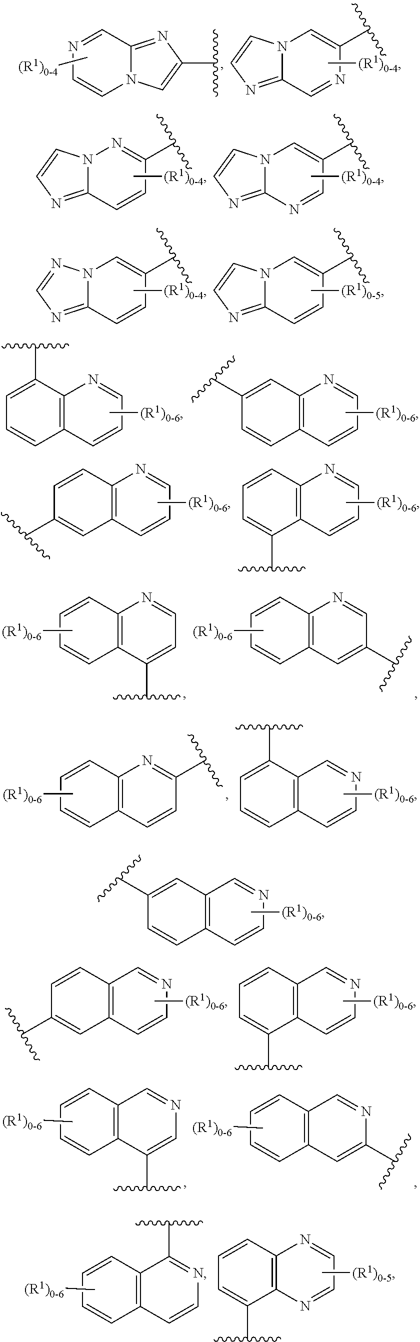

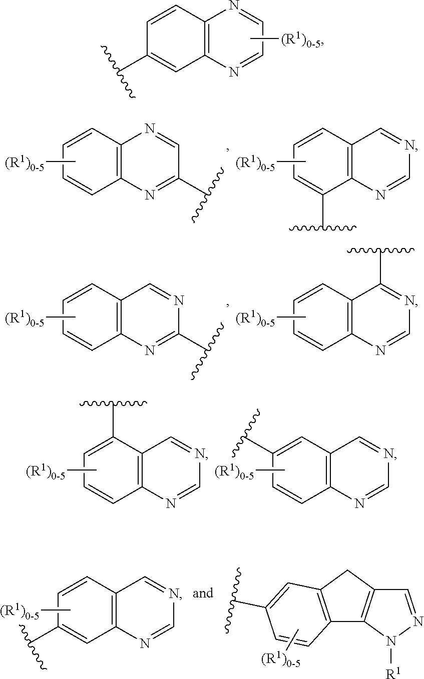

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

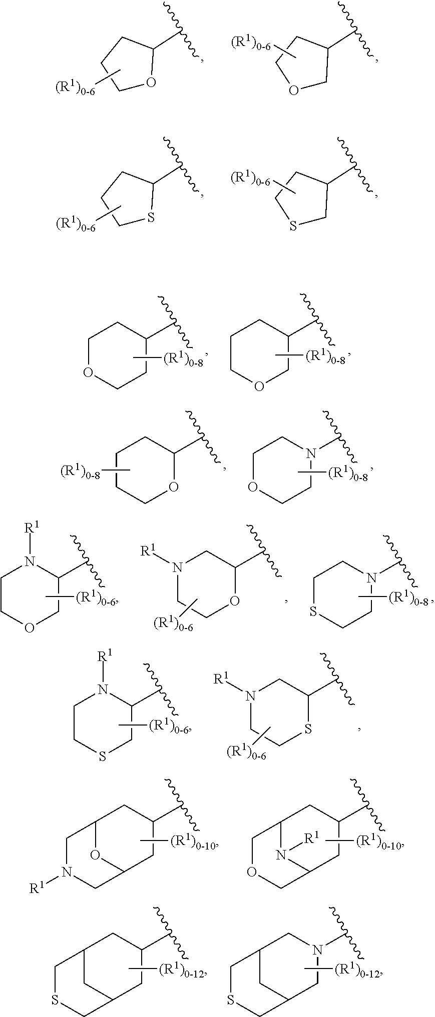

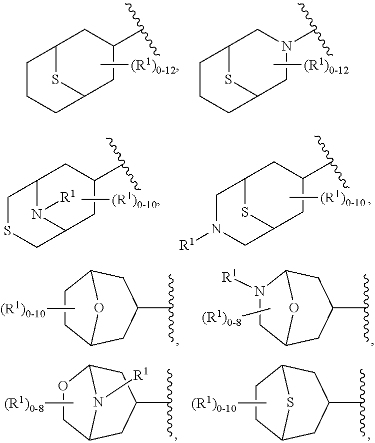

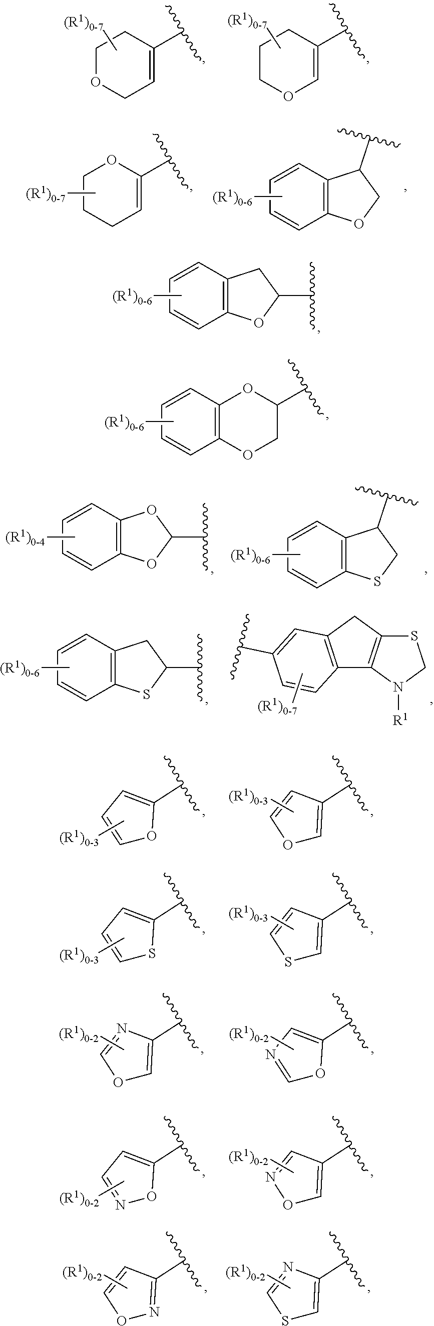

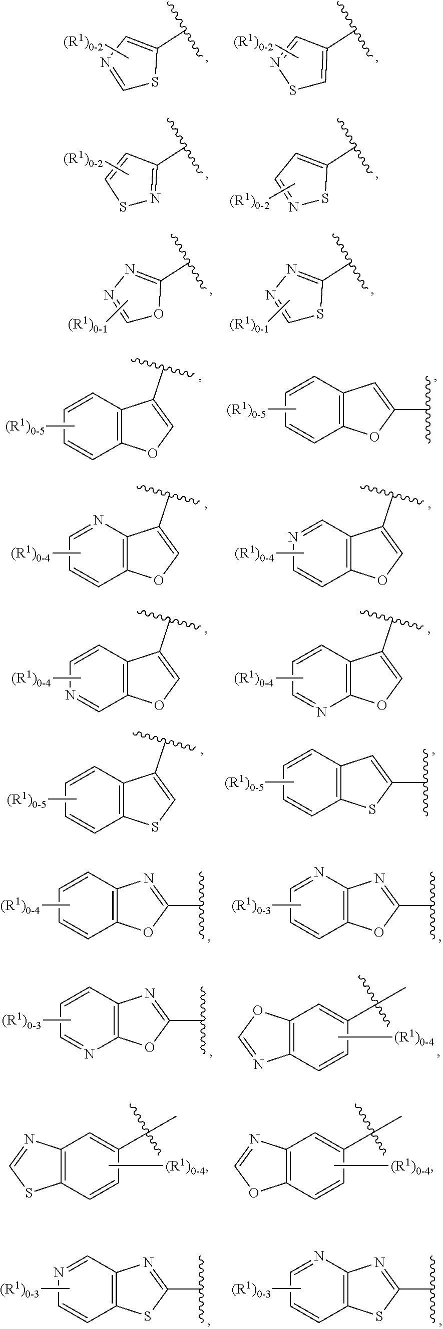

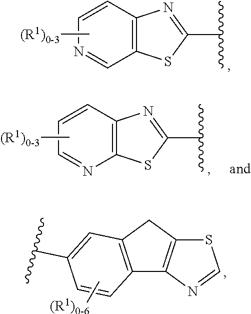

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, A is heterocyclyl. In some embodiments, A is a nitrogen-containing heterocyclyl. In some embodiments, A is a monocyclic nitrogen-containing heterocyclyl. In some embodiments, A is selected from

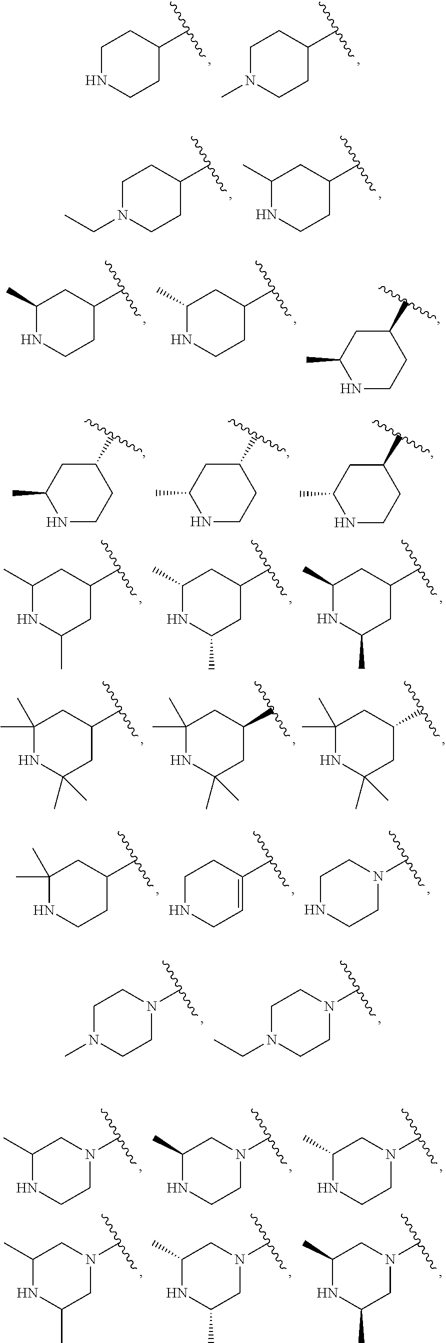

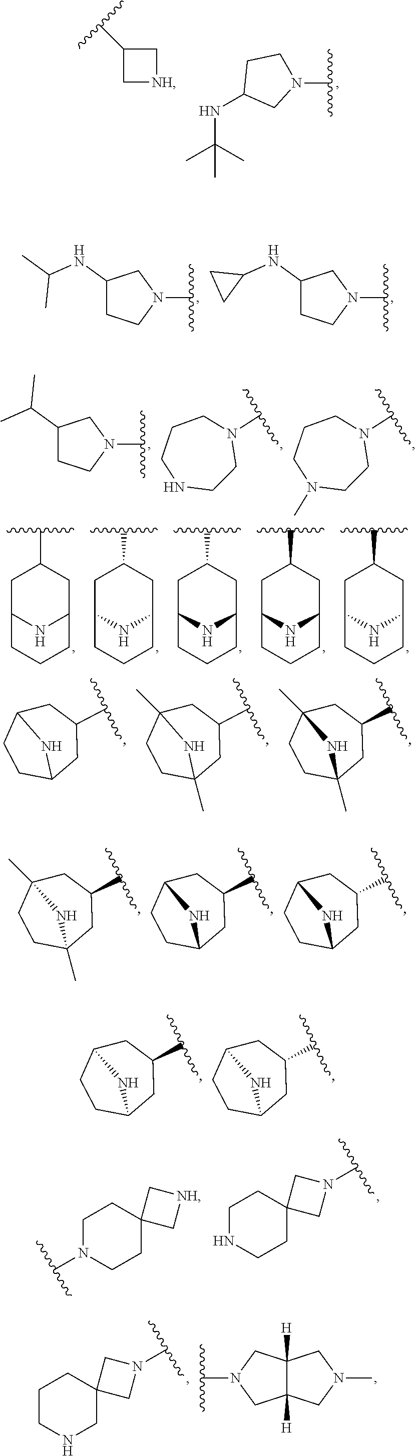

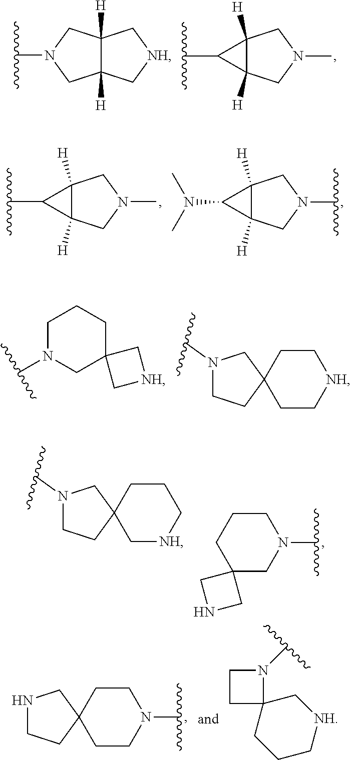

In some embodiments A is selected from

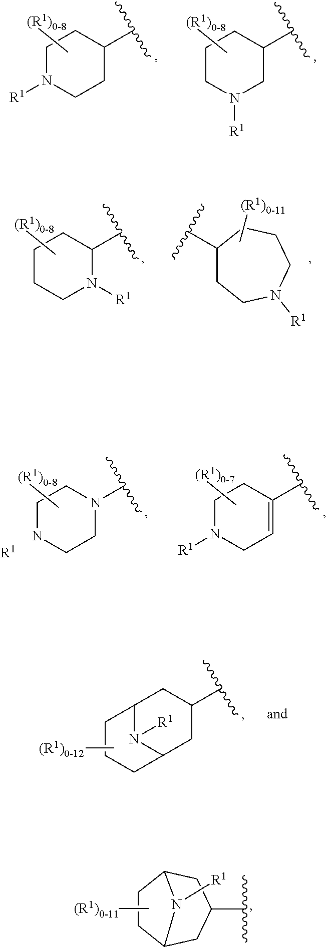

wherein R¹ is as defined herein.

In some embodiments, A is selected from,

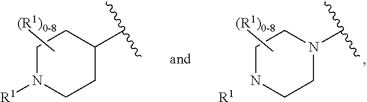

wherein R¹ is as defined herein.

In some embodiments, A is selected from

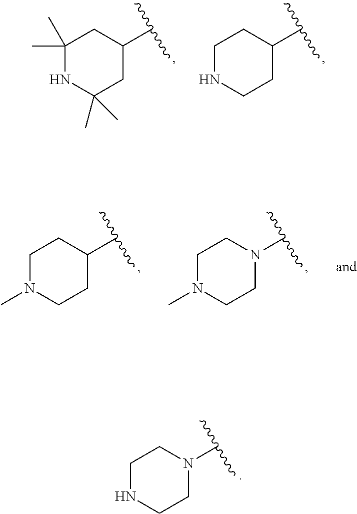

In some embodiments, A is heteroaryl. In some embodiments, A is a nitrogen-containing heteroaryl. In some embodiments, A is a bicyclic nitrogen-containing heteroaryl. In some embodiments, A is selected from

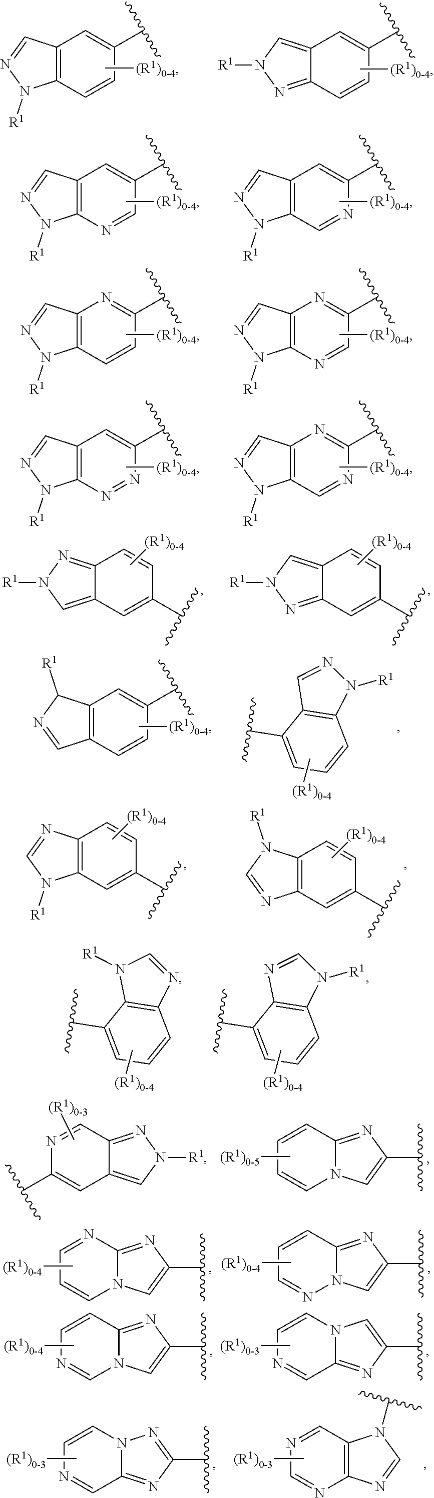

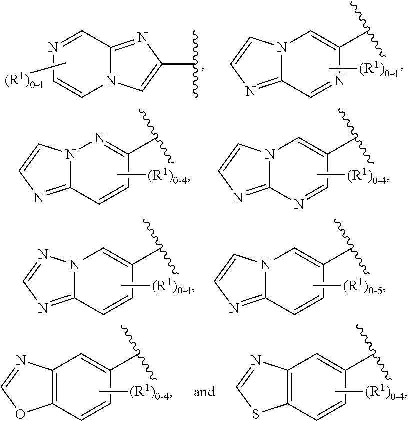

wherein R¹ is as defined herein.

In some embodiments, A is selected from

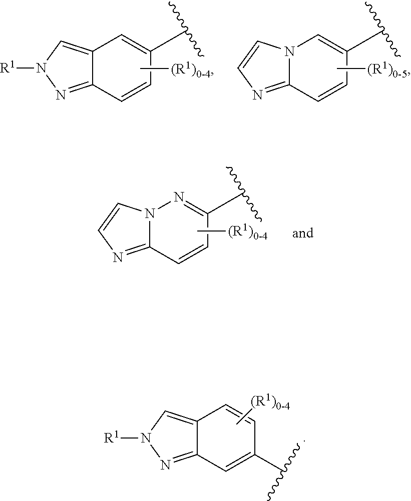

In some embodiments, A is

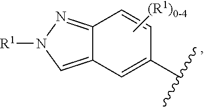

wherein R¹ is as defined herein. In some embodiments, A is selected from

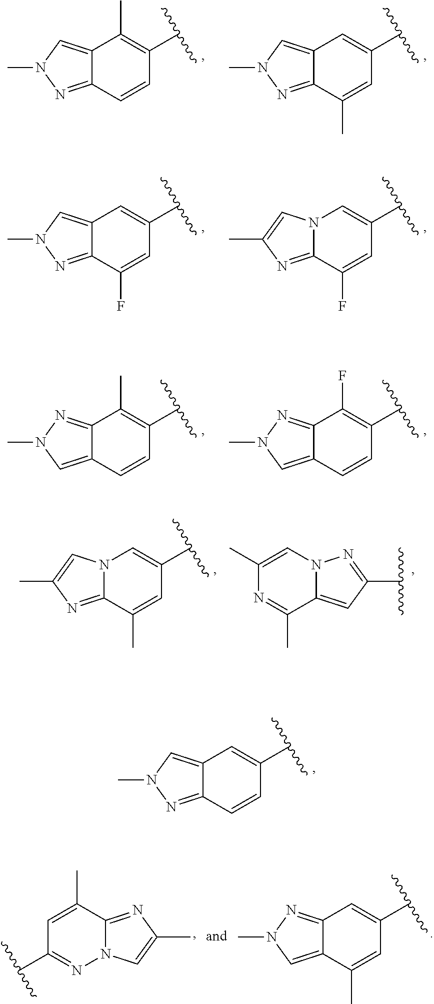

In some embodiments, A is

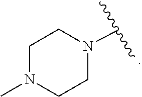 [SMILES: *N1CCN(C)CC1]

In some embodiments, A is

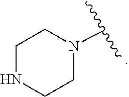 [SMILES: *N1CCNCC1]

In some embodiments, A is

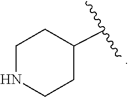 [SMILES: *C1CCNCC1]

In some embodiments, A is

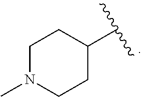 [SMILES: *C1CCN(C)CC1]

In some embodiments, A is

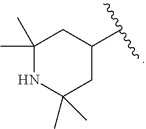 [SMILES: *C1CC(C)(C)NC(C)(C)C1]

In some embodiments, A is

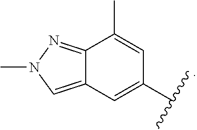 [SMILES: *c1cc(C)c2nn(C)cc2c1]

In some embodiments, A is

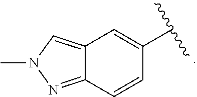 [SMILES: *c1ccc2nn(C)cc2c1]

In some embodiments, A is

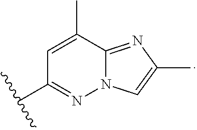 [SMILES: *c1cc(C)c2nc(C)cn2n1]

In some embodiments, A is

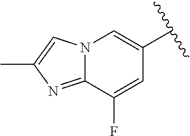 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

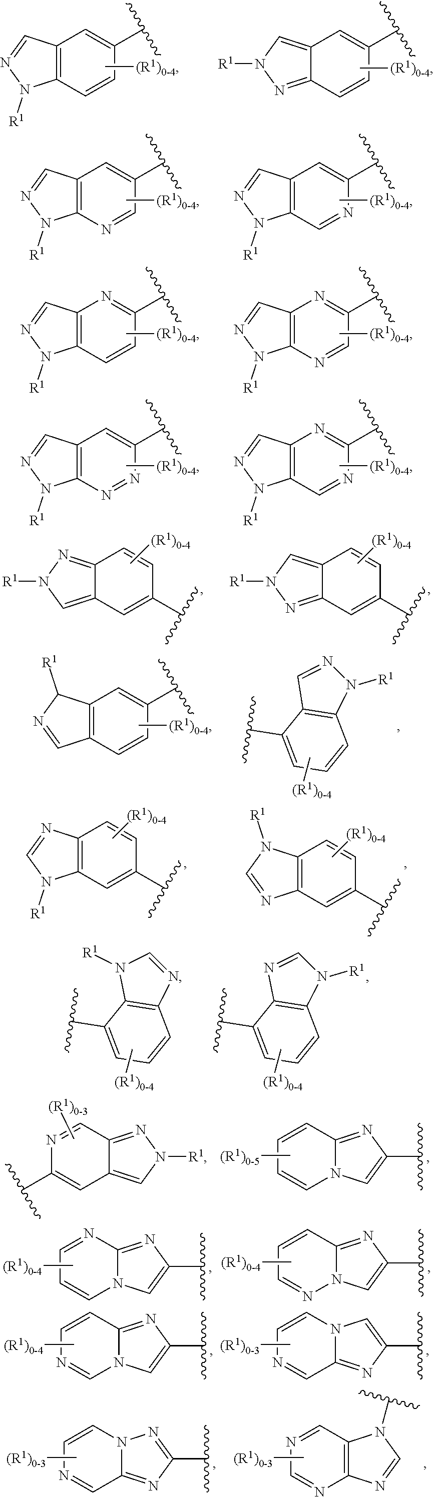

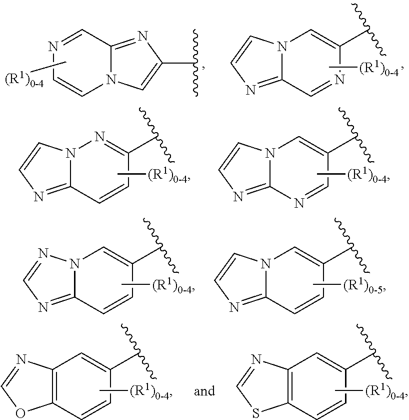

wherein R¹ is as defined herein.

In some embodiments, B is selected from

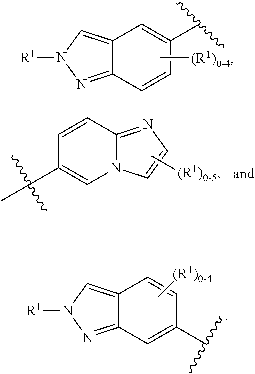

In some embodiments, B is

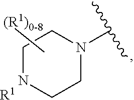

wherein R¹ is as defined herein.

In some embodiments, B is selected from

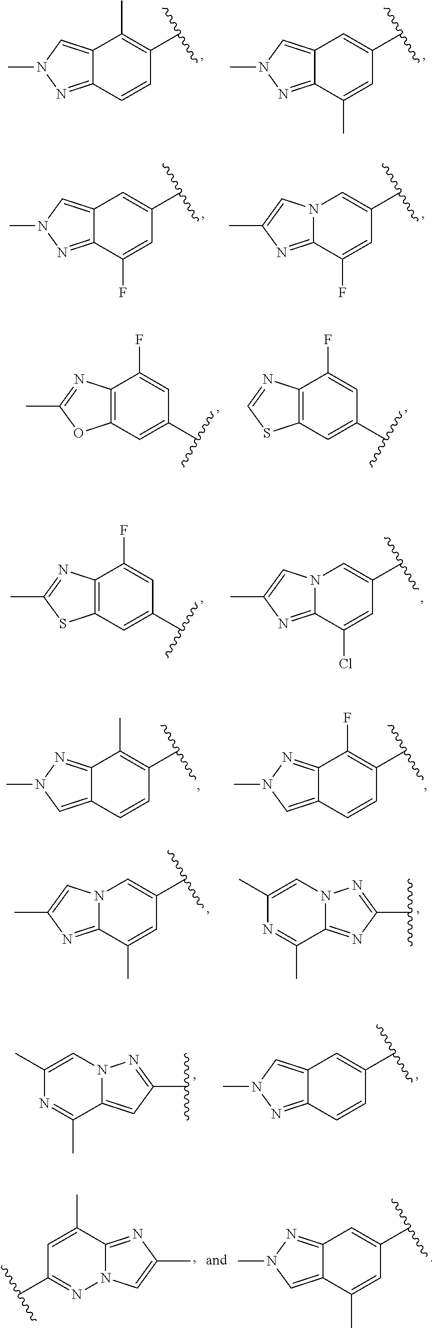

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl.

In some embodiments, B is selected from

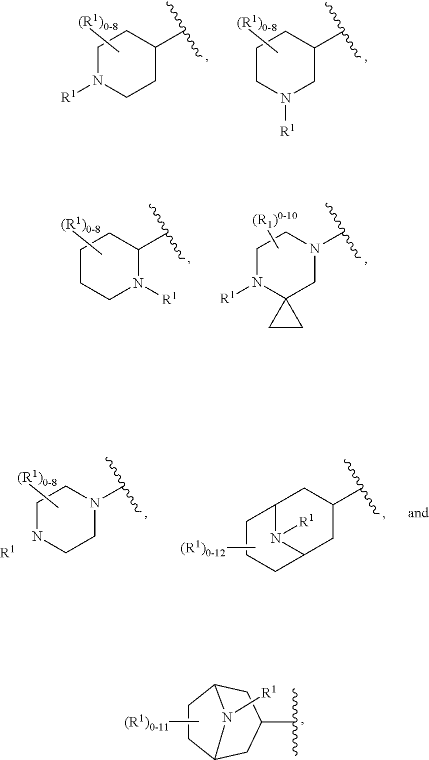

wherein R¹ is as defined herein. In some embodiments, B is selected from

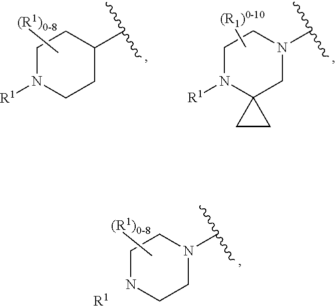

wherein R¹ is as defined herein. In some embodiments, B is selected from,

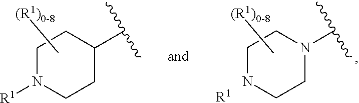

wherein R¹ is as defined herein.

In some embodiments B is selected from,

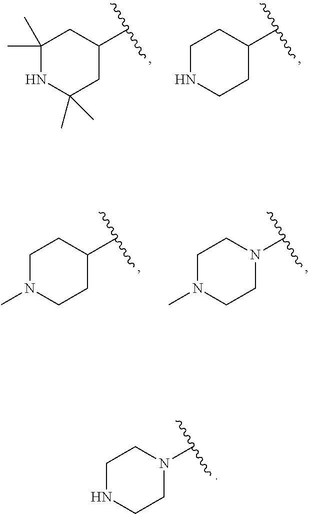

In some embodiments, B is

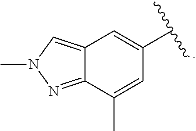 [SMILES: *c1cc(C)c2nn(C)cc2c1]

In some embodiments, B is

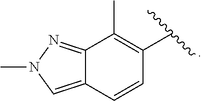 [SMILES: *c1ccc2cn(C)nc2c1C]

In some embodiments, B is

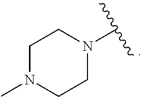 [SMILES: *N1CCN(C)CC1]

In some embodiments, B is

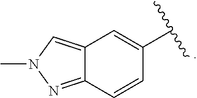 [SMILES: *c1ccc2nn(C)cc2c1]

In some embodiments, B is

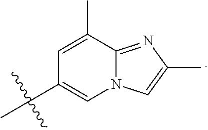 [SMILES: *c1cc(C)c2nc(C)cn2c1]

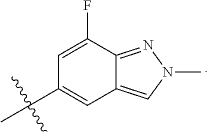 [SMILES: *c1cc(F)c2nn(C)cc2c1]

In some embodiments, B is

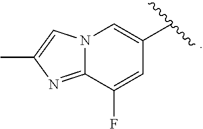 [SMILES: *c1cc(F)c2nc(C)cn2c1]

In some embodiments, B is

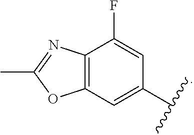 [SMILES: *c1cc(F)c2nc(C)oc2c1]

In some embodiments, B is

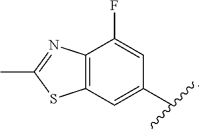 [SMILES: *c1cc(F)c2nc(C)sc2c1]

In some embodiments, B is

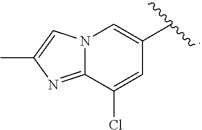 [SMILES: *c1cc(Cl)c2nc(C)cn2c1]

In some embodiments, B is

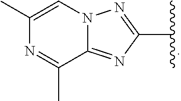 [SMILES: *c1nc2c(C)nc(C)cn2n1]

In some embodiments, B is

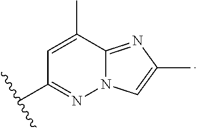 [SMILES: *c1cc(C)c2nc(C)cn2n1]

In some embodiments, B is

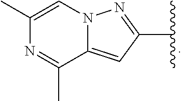 [SMILES: *c1cc2c(C)nc(C)cn2n1]

In some embodiments, B is

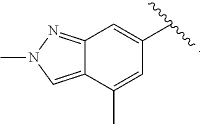 [SMILES: *c1cc(C)c2cn(C)nc2c1]

In some embodiments, B is

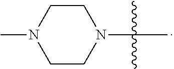 [SMILES: *N1CCN(C)CC1]

In some embodiments, B is

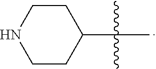 [SMILES: *C1CCNCC1]

As generally described for Formulas (I), (II), (III), (IV), (V), (VI), (VII), (VIII), and (IX), each of L¹ and L² may independently be absent or refer to a C₁-C₆-alkylene, C₁-C₆-heteroalkylene, —O—, —C(O)—, —N(R⁸)—, —N(R⁸)C(O)—, or —C(O)N(R⁸)— group, wherein each alkylene and heteroalkylene is optionally substituted with one or more R⁹. In some embodiments, L¹ is absent or C₁-C₆-heteroalkylene. In some embodiments, L¹ is absent. In some embodiments, L¹ is C₁-C₆-heteroalkylene (e.g., —N(CH₃)—). In some embodiments, L² is absent or C₁-C₆-heteroalkylene. In some embodiments, L² is absent. In some embodiments, L² is C₁-C₆-heteroalkylene (e.g., —N(CH₃)—).

As generally described for Formula (I), each of W, X, and Z may independently be N or C(R³). In some embodiments, W is C(R³) (e.g., CH). In some embodiments, W is N. In some embodiments, X is C(R³) (e.g., CH). In some embodiments, X is N. In some embodiments, Z is C(R³) (e.g., CH). In some embodiments, Z is N. In some embodiments, each of W and X is independently C(R³) (e.g., CH). In some embodiments, each of W and Z is independently C(R³) (e.g., CH). In some embodiments, each of X and Z is independently C(R³) (e.g., CH). In some embodiments, each of W, X, and Z is independently C(R³) (e.g., CH).

As generally described for Formula (I), Y may be N, N(R^{4a}), C(R^{4b}), or C(R^{4b})(R^{4c}), wherein the dashed lines in the ring comprising Y may be single or double bonds as valency permits. In some embodiments, Y is N(R^{4a}) or C(R^{4b}). In some embodiments, Y is N(R^{4a}) (e.g., NH). In some embodiments, Y is C(R^{4b}) (e.g., CH).

In some embodiments, W is C(R³) and Y is N(R^{4a}). In some embodiments, W is CH and Y is NH. In some embodiments, X is C(R³) and Y is N(R^{4a}). In some embodiments, X is CH and Y is NH. In some embodiments, Z is C(R³) and Y is N(R^{4a}). In some embodiments, Z is CH and Y is NH. In some embodiments, W and X are independently C(R³) and Y is N(R^{4a}). In some embodiments, W and X are independently C(R³) and Y is NH. In some embodiments, W and Z are independently C(R³) and Y is N(R^{4a}). In some embodiments, W and Z are independently C(R³) and Y is NH. In some embodiments, X and Z are independently C(R³) and Y is N(R^{4a}). In some embodiments, X and Z are independently C(R³) and Y is NH. In some embodiments, each of W, X, and Z is independently C(R³) and Y is N(R^{4a}). In some embodiments, each of W, X, and Z is independently CH and Y is NH.

In some embodiments, W is C(R³) and Y is N. In some embodiments, W is CH and Y is N. In some embodiments, X is C(R³) and Y is N. In some embodiments, X is CH and Y is N. In some embodiments, Z is C(R³) and Y is N. In some embodiments, Z is CH and Y is N. In some embodiments, W and X are independently C(R³) and Y is N. In some embodiments, W and X are independently C(R³) and Y is N. In some embodiments, W and Z are independently C(R³) and Y is N. In some embodiments, W and Z are independently C(R³) and Y is N. In some embodiments, X and Z are independently C(R³) and Y is N. In some embodiments, X and Z are independently C(R³) and Y is N. In some embodiments, each of W, X, and Z is independently C(R³) and Y is N. In some embodiments, each of W, X, and Z is independently CH and Y is N.

In some embodiments, R² is absent.

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹.

In some embodiments of Formula (II), A is a bicyclic heteroaryl and B is a monocyclic heterocyclyl. In some embodiments of Formula (II), Z is N. In some embodiments of Formula (II), each of W, X, and Z is not independently C(R³), e.g., (CH). In some embodiments of Formula (II), the compound is not a compound disclosed in WO 2020/004594.

In some embodiments, for Formula (III), A is a bicyclic heteroaryl not containing oxygen. In some embodiments, A is a bicyclic heteroaryl substituted by one or more R¹, wherein R¹ is not halo. In some embodiments, A is not

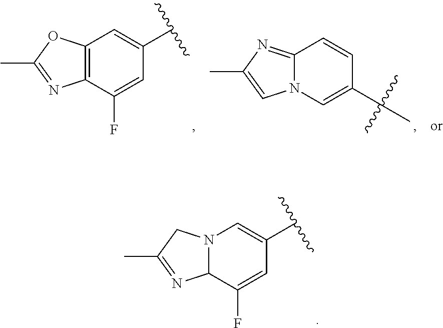 [SMILES: *c1cc(F)c2nc(C)oc2c1 ; *c1ccc2nc(C)cn2c1 ; *C1=CN2CC(C)=NC2C(F)=C1]

In some embodiments, B is a nitrogen-containing heterocyclyl optionally substituted with one or more R¹, wherein R¹ is not cycloalkyl (e.g., cyclopropyl). In some embodiments, B is unsubstituted piperidinyl (e.g., 0 R¹). In some embodiments, B is not

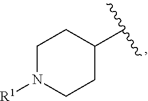

wherein R¹ is C₁-C₆ alkyl (e.g., methyl) or cycloalkyl (e.g., cyclopropyl). In some embodiments, B is

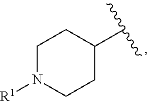

wherein R¹ is hydrogen. In some embodiments, B is not

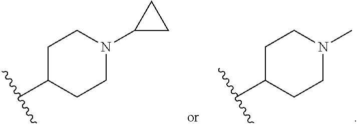

In some embodiments, B is not

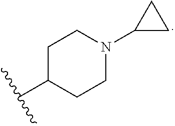 [SMILES: *C1CCN(C2CC2)CC1]

In some embodiments, X is C(R³), wherein R³ is halo. In some embodiments, X is CF. In some embodiments, the compound of Formula (III) is not a compound disclosed in WO 2020/004594. In some embodiments, the compound of Formula (III) is not a compound selected from

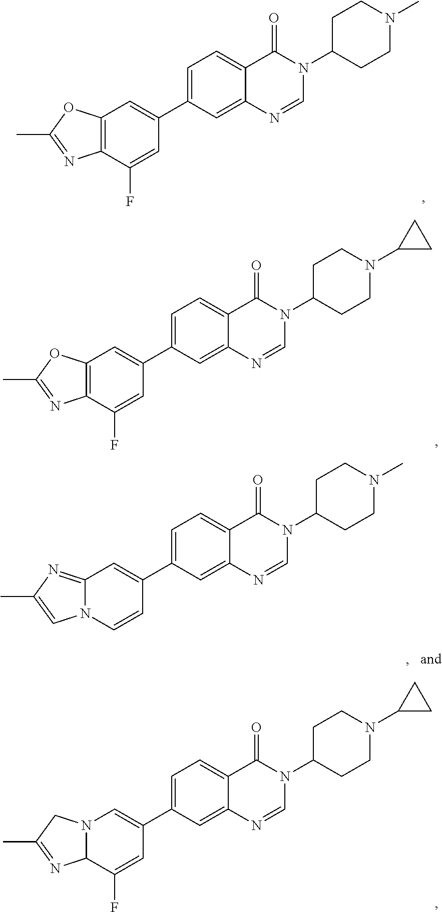

or a pharmaceutically acceptable salt thereof.

In some embodiments, the present disclosure features a compound of Formula (I-i):

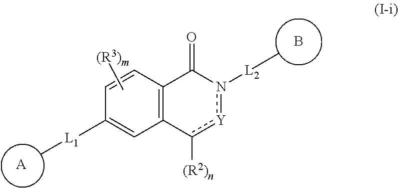

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁷; Y is N, C(R^{6a}), or C(R^{6a})(R⁶¹), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen or C₁-C₆-alkyl; R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; R^{6a} and R^{6b} is independently hydrogen, C₁-C₆-alkyl, C₁-C₆-heteroalkyl, C₁-C₆-haloalkyl, or halo; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; n is 0, 1, or 2; m is 0, 1, 2, or 3; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

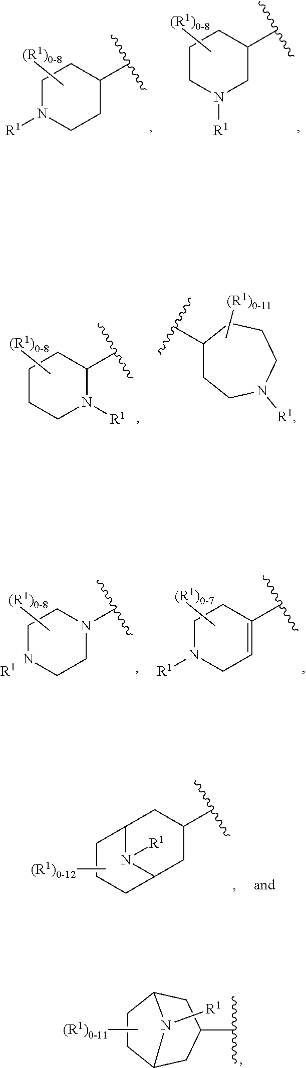

wherein R¹ is as defined herein.

In some embodiments, A is selected from

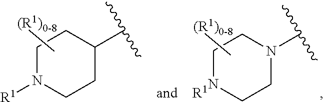

wherein R¹ is as defined herein.

In some embodiments A is selected from

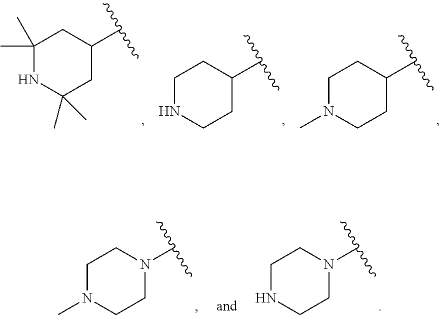

In some embodiments, A is heteroaryl. In some embodiments, A is a nitrogen-containing heteroaryl. In some embodiments, A is a bicyclic nitrogen-containing heteroaryl.

In some embodiments, A is selected from

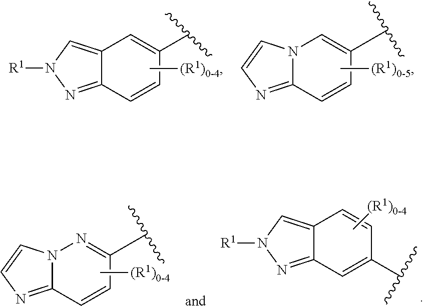

In some embodiments, A is

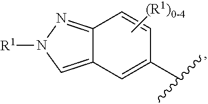

wherein R¹ is as defined herein. In some embodiments, A is selected from

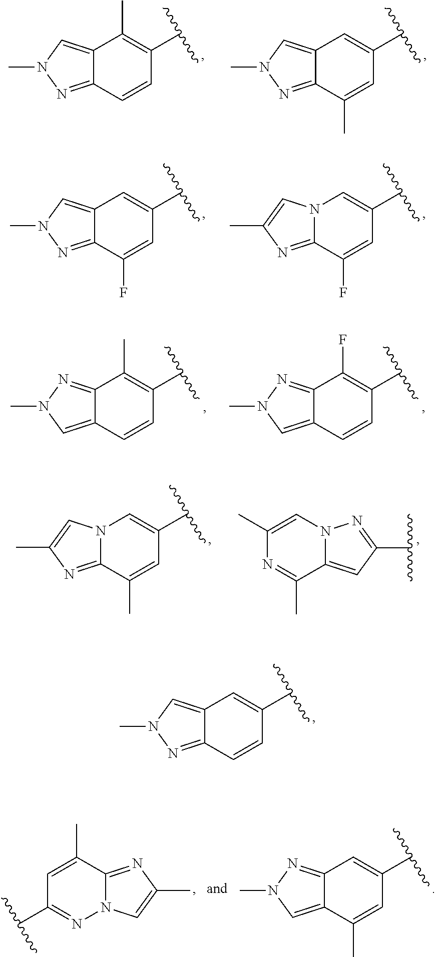

In some embodiments A is

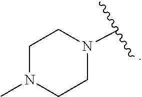 [SMILES: *N1CCN(C)CC1]

In some embodiments, A is

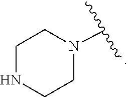 [SMILES: *N1CCNCC1]

In some embodiments, A is

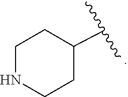 [SMILES: *C1CCNCC1]

In some embodiments, A is

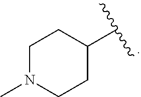 [SMILES: *C1CCN(C)CC1]

In some embodiments, A is

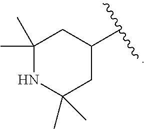 [SMILES: *C1CC(C)(C)NC(C)(C)C1]

In some embodiments, A is

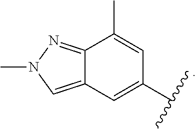 [SMILES: *c1cc(C)c2nn(C)cc2c1]

In some embodiments, A is

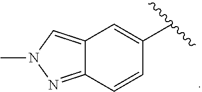 [SMILES: *c1ccc2nn(C)cc2c1]

In some embodiments, A is

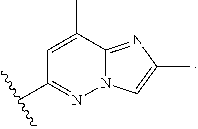 [SMILES: *c1cc(C)c2nc(C)cn2n1]

In some embodiments, A is

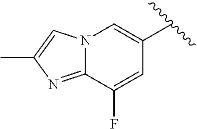 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

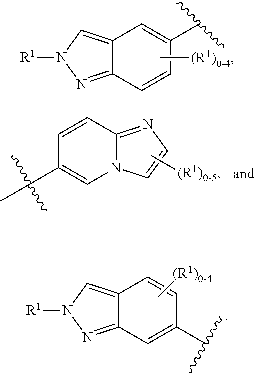

In some embodiments, B is

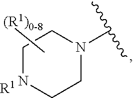

wherein R¹ is as defined herein.

In some embodiments, B is selected from

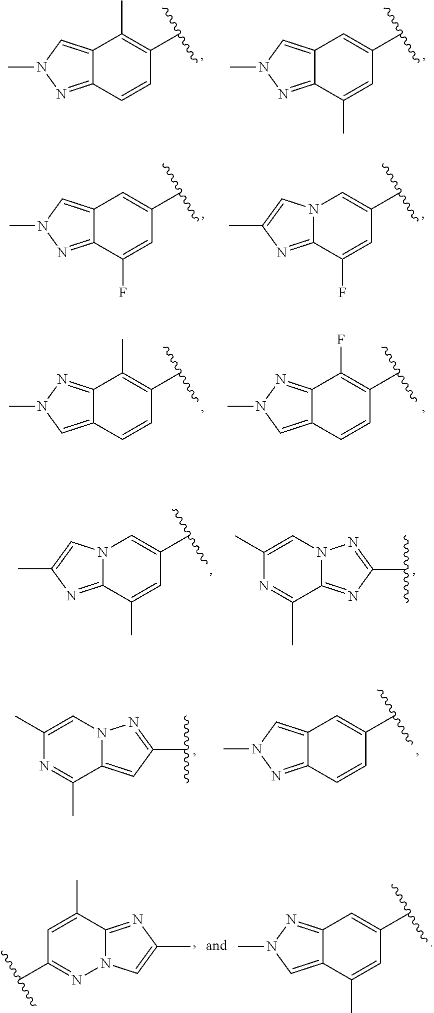

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

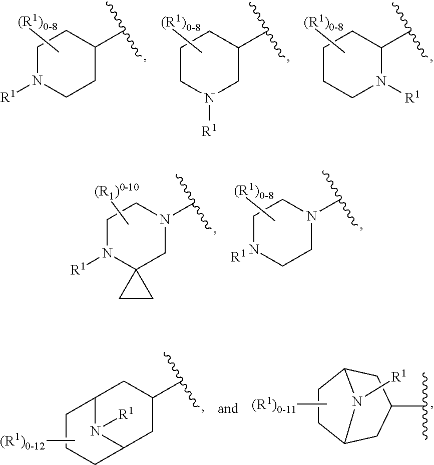

wherein R¹ is as defined herein. In some embodiments, B is selected from

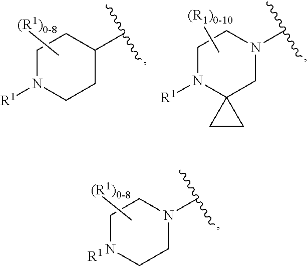

wherein R¹ is as defined herein. In some embodiments, B is selected from,

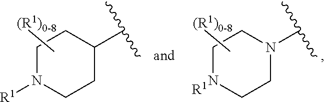

wherein R¹ is as defined herein.

In some embodiments, B is selected from

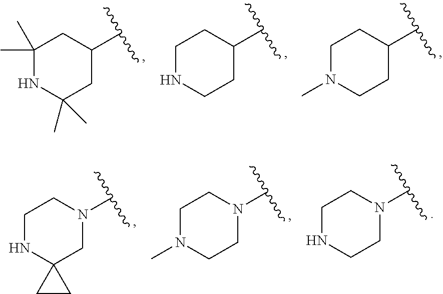

In some embodiments, B is

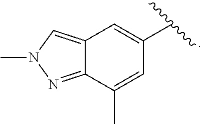 [SMILES: *c1cc(C)c2nn(C)cc2c1]

In some embodiments, B is

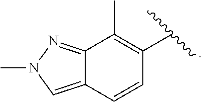 [SMILES: *c1ccc2cn(C)nc2c1C]

In some embodiments, B is

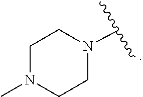 [SMILES: *N1CCN(C)CC1]

In some embodiments, B is

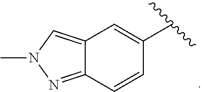 [SMILES: *c1ccc2nn(C)cc2c1]

In some embodiments, B is

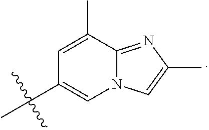 [SMILES: *c1cc(C)c2nc(C)cn2c1]

In some embodiments, B is

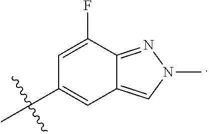 [SMILES: *c1cc(F)c2nn(C)cc2c1]

In some embodiments, B is

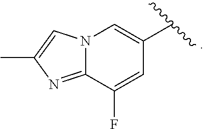 [SMILES: *c1cc(F)c2nc(C)cn2c1]

In some embodiments, B is

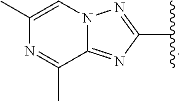 [SMILES: *c1nc2c(C)nc(C)cn2n1]

In some embodiments, B is

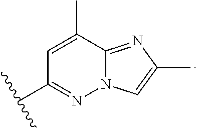 [SMILES: *c1cc(C)c2nc(C)cn2n1]

In some embodiments, B is

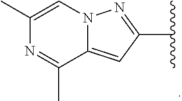 [SMILES: *c1cc2c(C)nc(C)cn2n1]

In some embodiments, B is

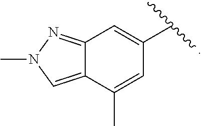 [SMILES: *c1cc(C)c2cn(C)nc2c1]

In some embodiments, B is

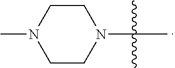 [SMILES: *N1CCN(C)CC1]

In some embodiments, B is

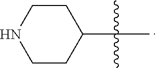 [SMILES: *C1CCNCC1]

In some embodiments, B is

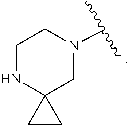 [SMILES: *N1CCNC2(CC2)C1]

In some embodiments, the compound of Formula (I) is Formula (I-a):

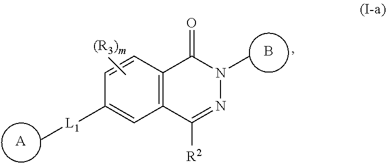

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁷; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen or C₁-C₆-alkyl; R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; n is 0, 1, or 2; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

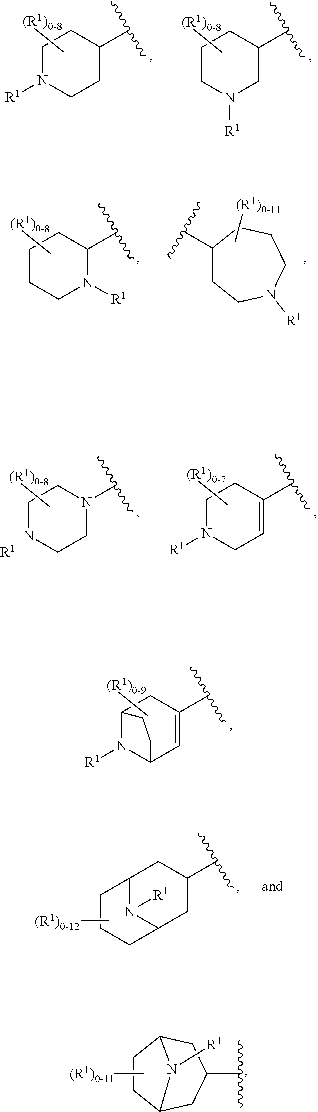

wherein R¹ is as defined herein.

In some embodiments, A is selected from,

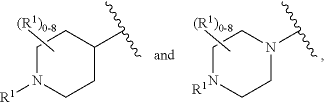

wherein R¹ is as defined herein.

In some embodiments, A is selected from

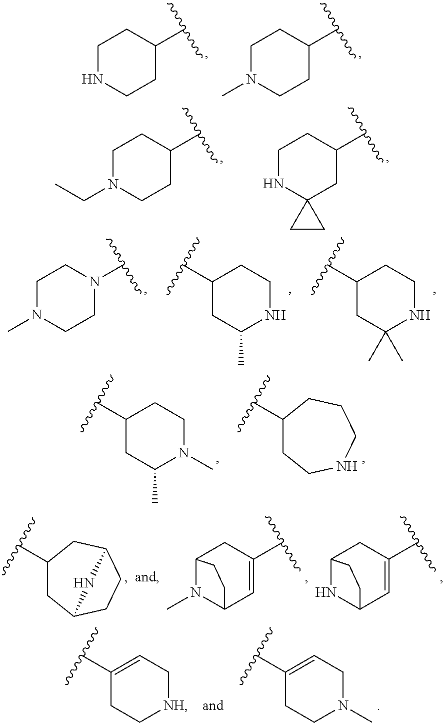

In some embodiments, A is heteroaryl. In some embodiments, A is a nitrogen-containing heteroaryl. In some embodiments, A is a bicyclic nitrogen-containing heteroaryl.

In some embodiments, A is selected from

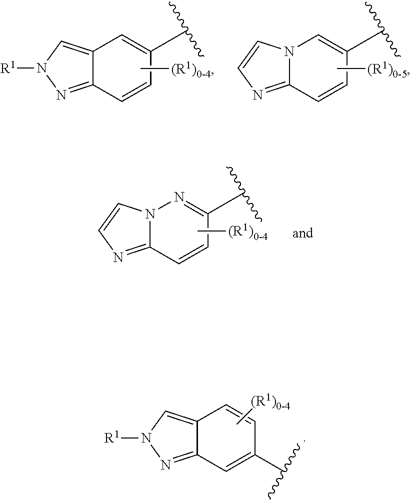

In some embodiments, A is

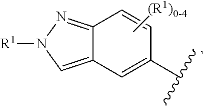

wherein R¹ is as defined herein. In some embodiments, A is selected from

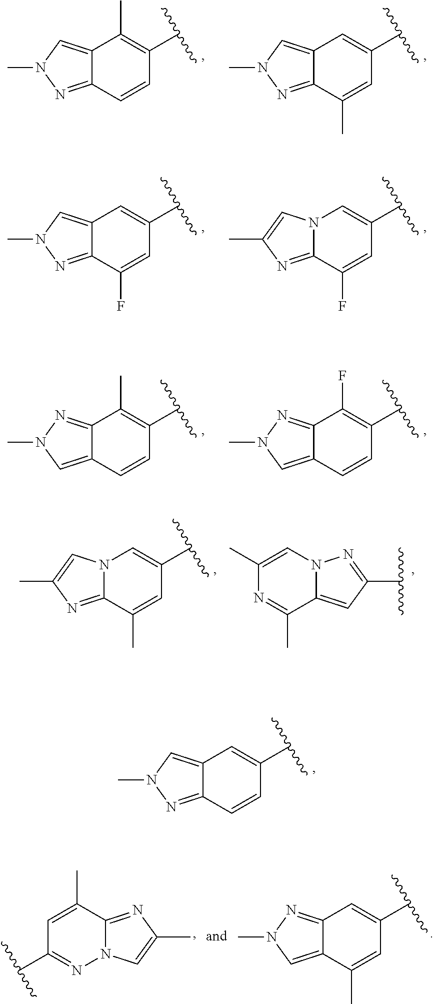

In some embodiments, A is

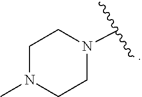 [SMILES: *N1CCN(C)CC1]

In some embodiments, A is

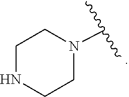 [SMILES: *N1CCNCC1]

In some embodiments, A is

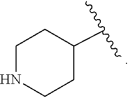 [SMILES: *C1CCNCC1]

In some embodiments, A is

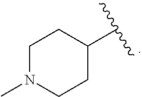 [SMILES: *C1CCN(C)CC1]

In some embodiments, A is

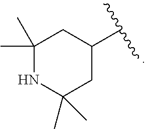 [SMILES: *C1CC(C)(C)NC(C)(C)C1]

In some embodiments, A is

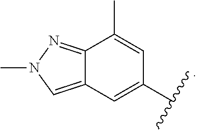 [SMILES: *c1cc(C)c2nn(C)cc2c1]

In some embodiments, A is

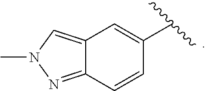 [SMILES: *c1ccc2nn(C)cc2c1]

In some embodiments, A is

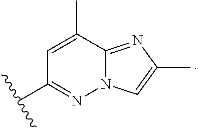 [SMILES: *c1cc(C)c2nc(C)cn2n1]

In some embodiments, A is

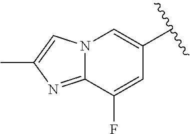 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

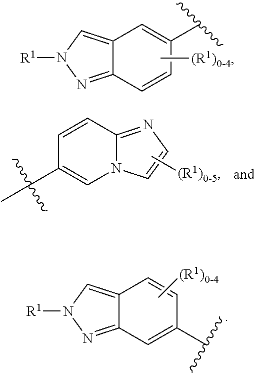

In some embodiments, B is

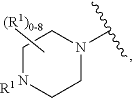

wherein R¹ is as defined herein.

In some embodiments, B is selected from

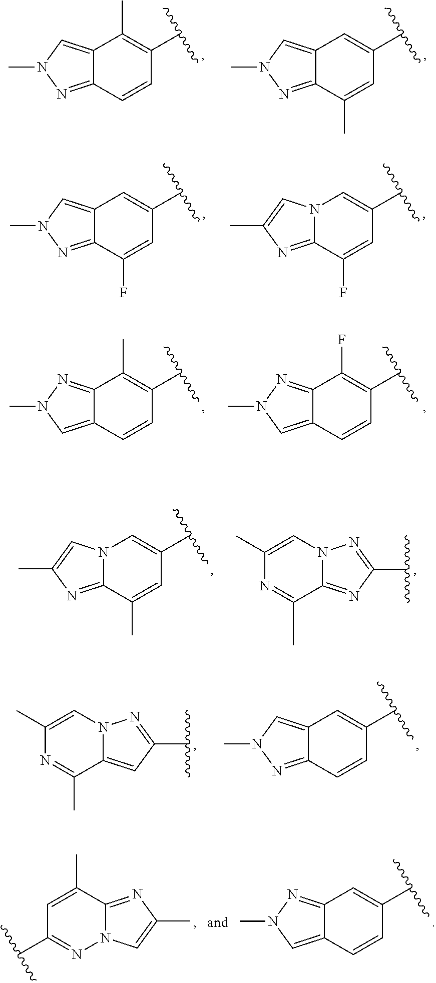

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected

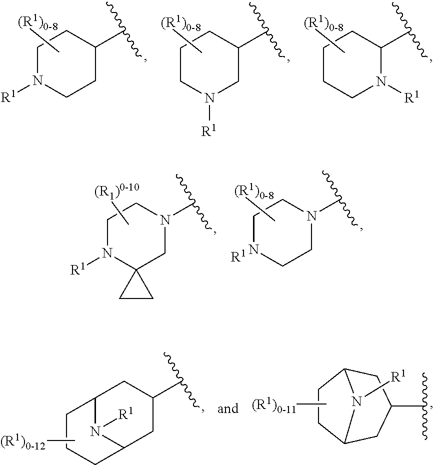

wherein R¹ is as defined herein. In some embodiments, B is selected
from

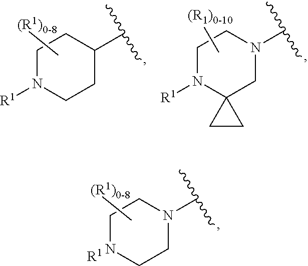

wherein R¹ is as defined herein. In some embodiments, B is selected from,

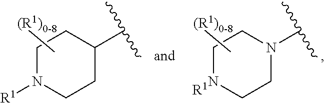

wherein R¹ is as defined herein.

In some embodiments, B is selected from

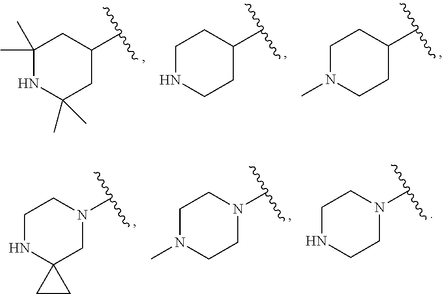

In some embodiments, B is

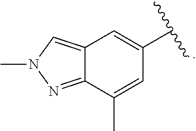 [SMILES: *c1cc(C)c2nn(C)cc2c1]

In some embodiments, B is

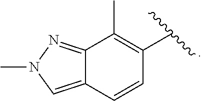 [SMILES: *c1ccc2cn(C)nc2c1C]

In some embodiments, B is

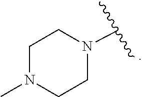 [SMILES: *N1CCN(C)CC1]

In some embodiments, B is

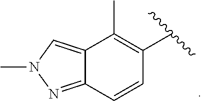 [SMILES: *c1ccc2nn(C)cc2c1C]

In some embodiments, B is

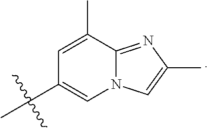 [SMILES: *c1cc(C)c2nc(C)cn2c1]

In some embodiments, B is

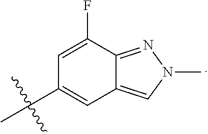 [SMILES: *c1cc(F)c2nn(C)cc2c1]

In some embodiments, B is

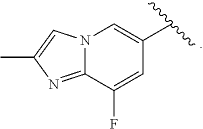 [SMILES: *c1cc(F)c2nc(C)cn2c1]

In some embodiments, B is

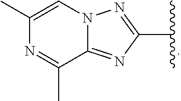 [SMILES: *c1nc2c(C)nc(C)cn2n1]

In some embodiments, B is

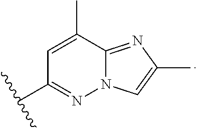 [SMILES: *c1cc(C)c2nc(C)cn2n1]

In some embodiments, B is

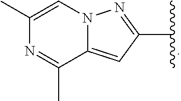 [SMILES: *c1cc2c(C)nc(C)cn2n1]

In some embodiments, B is

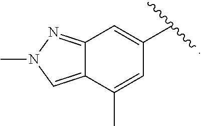 [SMILES: *c1cc(C)c2cn(C)nc2c1]

In some embodiments, B is

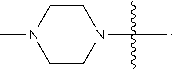 [SMILES: *N1CCN(C)CC1]

In some embodiments, B is

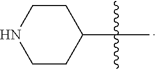 [SMILES: *C1CCNCC1]

In some embodiments, B is

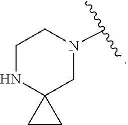 [SMILES: *N1CCNC2(CC2)C1]

In some embodiments, the compound of Formula (I) is Formula (I-b):

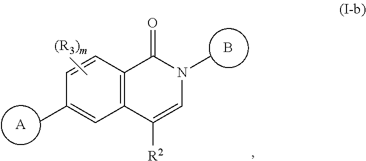

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁷; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen or C₁-C₆-alkyl; R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; m is 0, 1, 2, or 3; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

In some embodiments, B is

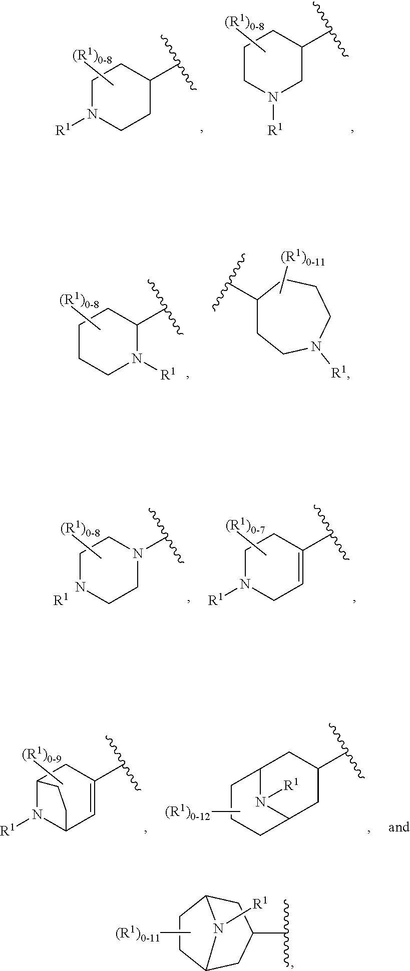

wherein R¹ is as defined herein.

In some embodiments, A is selected from,

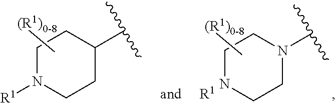

wherein R¹ is as defined herein.

In some embodiments, A is selected from

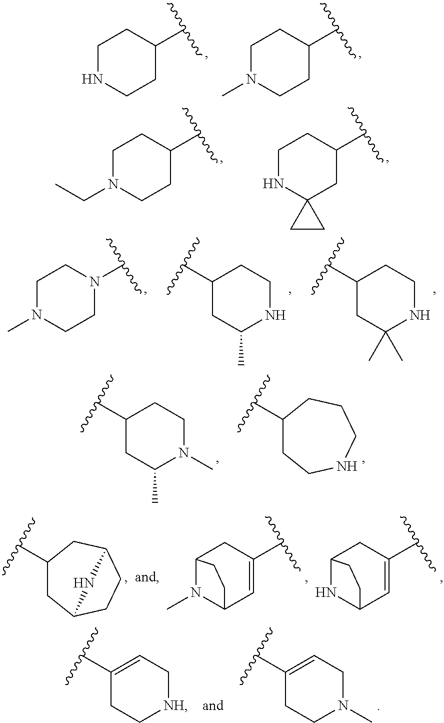

In some embodiments, A is heteroaryl. In some embodiments, A is a nitrogen-containing heteroaryl. In some embodiments, A is a bicyclic nitrogen-containing heteroaryl.

In some embodiments, A is selected from

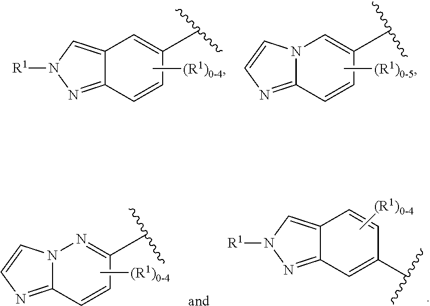

In some embodiments, A is

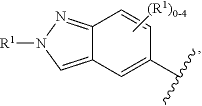

wherein R¹ is as defined herein. In some embodiments, A is selected from

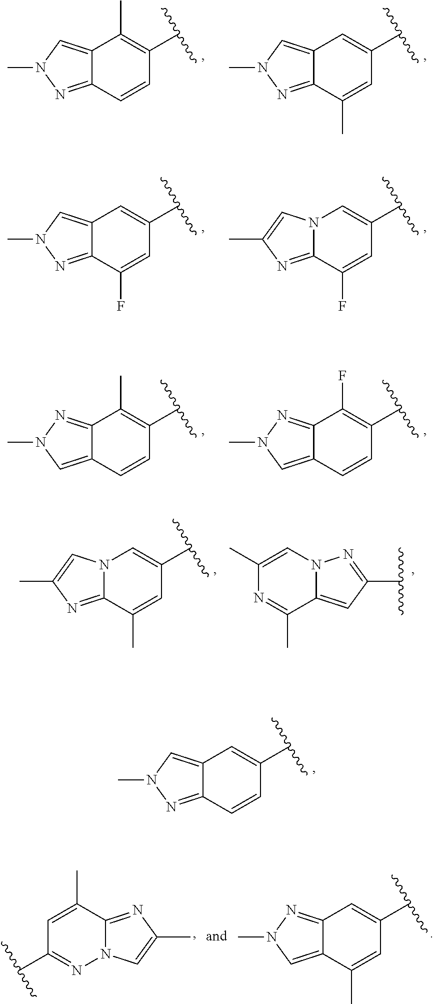

In some embodiments, A is

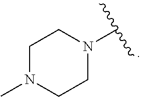 [SMILES: *N1CCN(C)CC1]

In some embodiments, A is

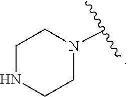 [SMILES: *N1CCNCC1]

In some embodiments, A is

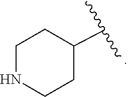 [SMILES: *C1CCNCC1]

In some embodiments, A is

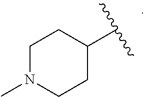 [SMILES: *C1CCN(C)CC1]

In some embodiments, A is

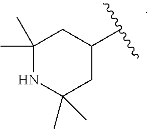 [SMILES: *C1CC(C)(C)NC(C)(C)C1]

In some embodiments, A is

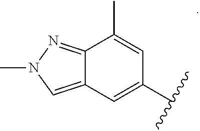 [SMILES: *c1cc(C)c2nn(C)cc2c1]

In some embodiments, A is

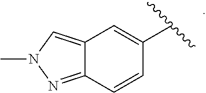 [SMILES: *c1ccc2nn(C)cc2c1]

In some embodiments, A is

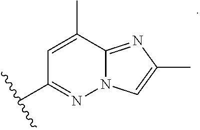 [SMILES: *c1cc(C)c2nc(C)cn2n1]

In some embodiments, A is

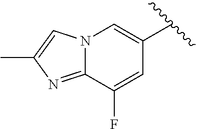 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

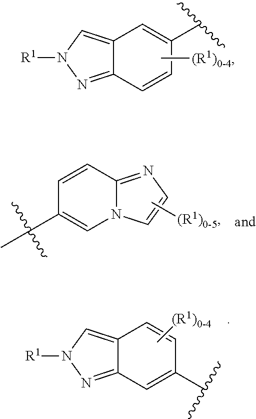

In some embodiments, B is

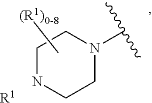

wherein R¹ is as defined herein.

In some embodiments, B is selected from

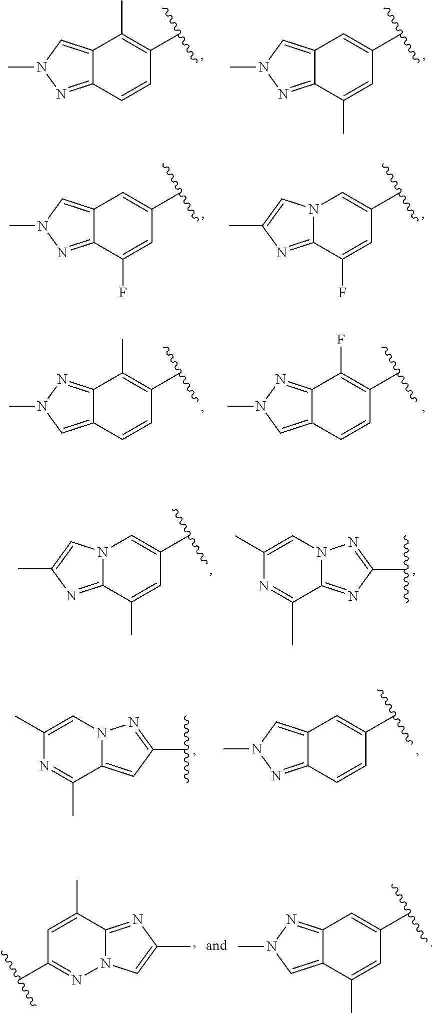

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

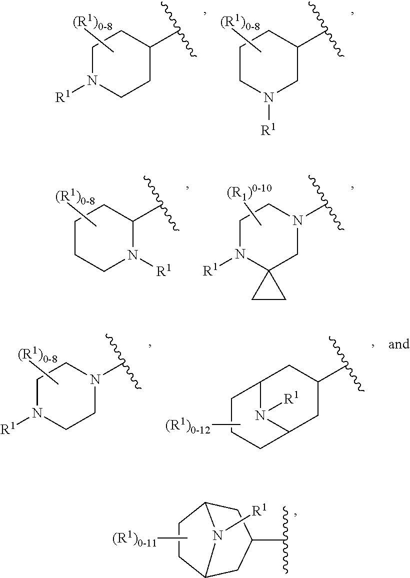

wherein R¹ is as defined herein. In some embodiments, B is selected from

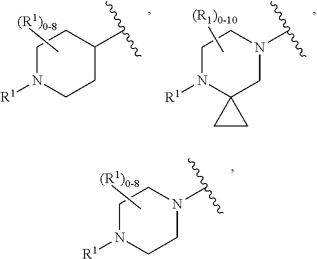

wherein R¹ is as defined herein. In some embodiments, B is selected from,

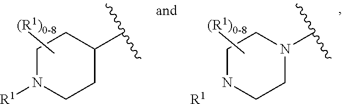

wherein R¹ is as defined herein.

In some embodiments, B is selected from

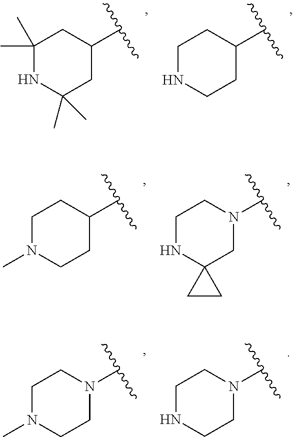

In some embodiments, B is

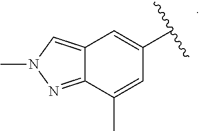 [SMILES: *c1cc(C)c2nn(C)cc2c1]

In some embodiments, B is

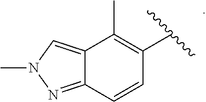 [SMILES: *c1ccc2nn(C)cc2c1C]

In some embodiments, B is

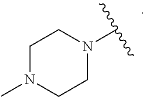 [SMILES: *N1CCN(C)CC1]

In some embodiments, B is

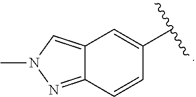 [SMILES: *c1ccc2nn(C)cc2c1]

In some embodiments, B is

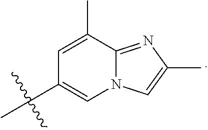 [SMILES: *c1cc(C)c2nc(C)cn2c1]

In some embodiments, B is

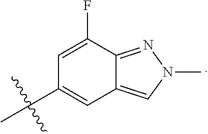 [SMILES: *c1cc(F)c2nn(C)cc2c1]

In some embodiments, B is

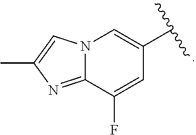 [SMILES: *c1cc(F)c2nc(C)cn2c1]

In some embodiments, B is

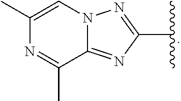 [SMILES: *c1nc2c(C)nc(C)cn2n1]

In some embodiments, B is

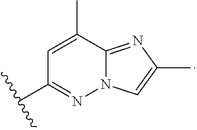 [SMILES: *c1cc(C)c2nc(C)cn2n1]

In some embodiments, B is

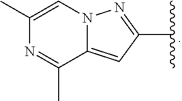 [SMILES: *c1cc2c(C)nc(C)cn2n1]

In some embodiments, B is

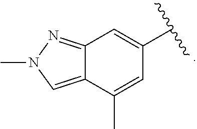 [SMILES: *c1cc(C)c2cn(C)nc2c1]

In some embodiments, B is

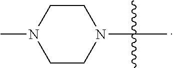 [SMILES: *N1CCN(C)CC1]

In some embodiments, B is

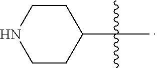 [SMILES: *C1CCNCC1]

In some embodiments, B is

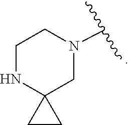 [SMILES: *N1CCNC2(CC2)C1]

In some embodiments, the compound of Formula (I) is Formula (I-c):

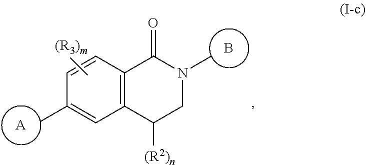

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁷; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen or C₁-C₆-alkyl; R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; n is 0, 1, or 2; m is 0, 1, 2, or 3; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

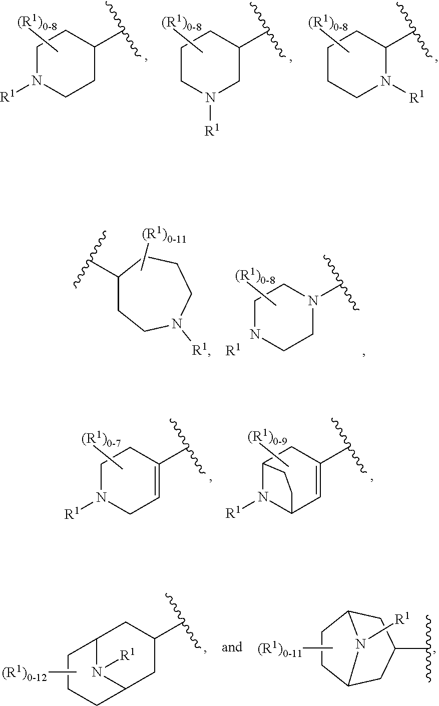

wherein R¹ is as defined herein.

In some embodiments, A is selected from,

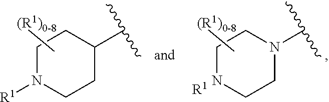

wherein R¹ is as defined herein.

In some embodiments, A is selected from

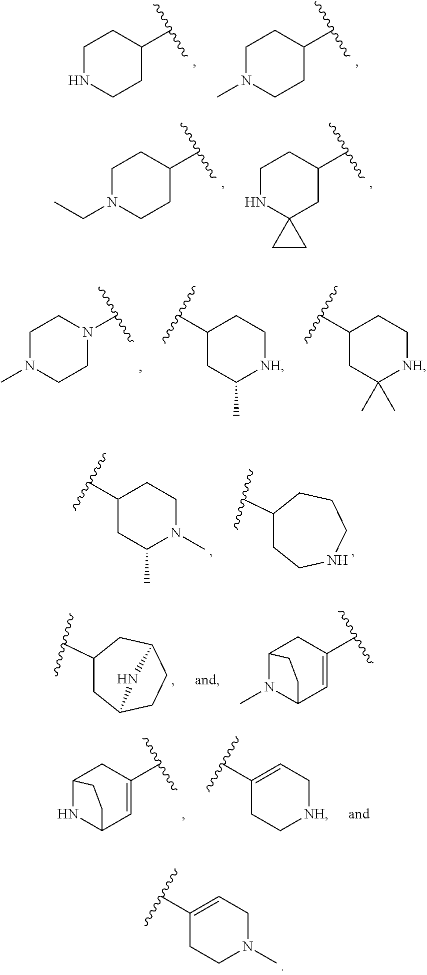

In some embodiments, A is heteroaryl. In some embodiments, A is a nitrogen-containing heteroaryl. In some embodiments, A is a bicyclic nitrogen-containing heteroaryl.

In some embodiments, A is selected from

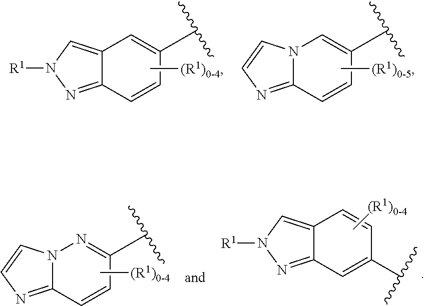

In some embodiments, A is

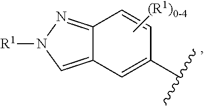

wherein R¹ is as defined herein. In some embodiments, A is selected from

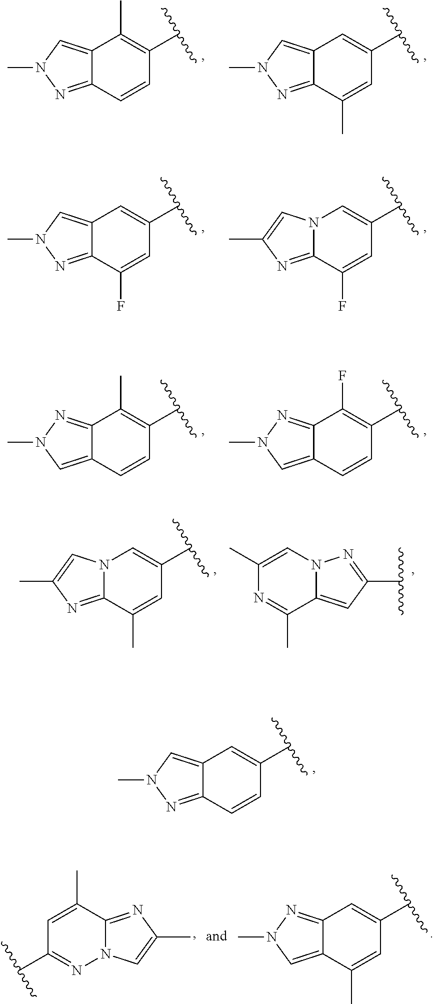

In some embodiments A is

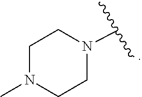 [SMILES: *N1CCN(C)CC1]

In some embodiments A is

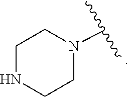 [SMILES: *N1CCNCC1]

In some embodiments, A is

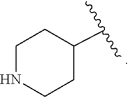 [SMILES: *C1CCNCC1]

In some embodiments, A is

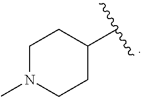 [SMILES: *C1CCN(C)CC1]

In some embodiments, A is

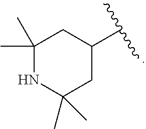 [SMILES: *C1CC(C)(C)NC(C)(C)C1]

In some embodiments, A is

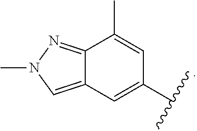 [SMILES: *c1cc(C)c2nn(C)cc2c1]

In some embodiments, A is

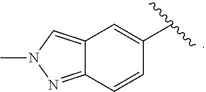 [SMILES: *c1ccc2nn(C)cc2c1]

In some embodiments A is

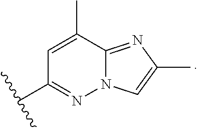 [SMILES: *c1cc(C)c2nc(C)cn2n1]

In some embodiments, A is

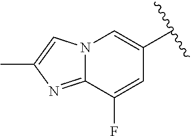 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

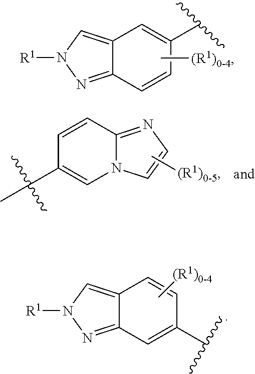

In some embodiments, B is

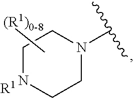

wherein R¹ is as defined herein.

In some embodiments, B is selected from

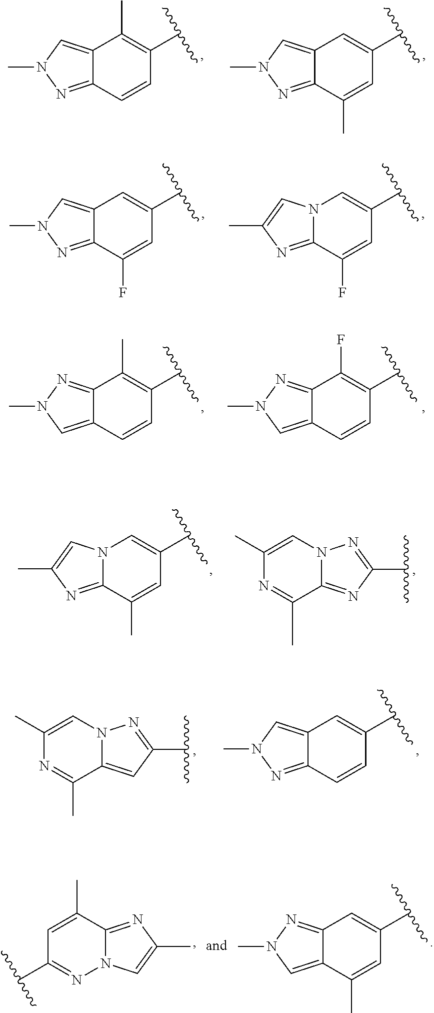

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

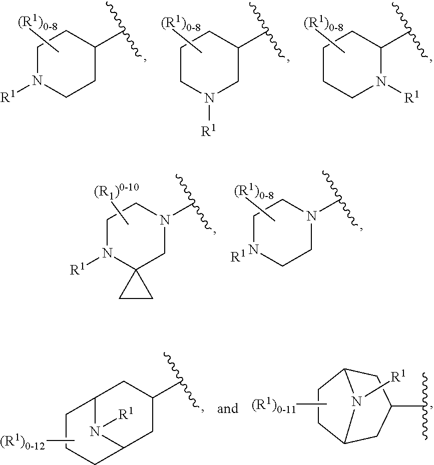

wherein R¹ is as defined herein. In some embodiments, B is selected from

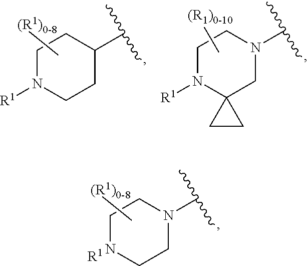

wherein R¹ is as defined herein. In some embodiments, B is selected from,

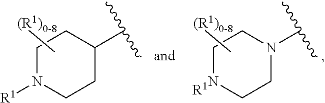

wherein R¹ is as defined herein.

In some embodiments, B is selected from

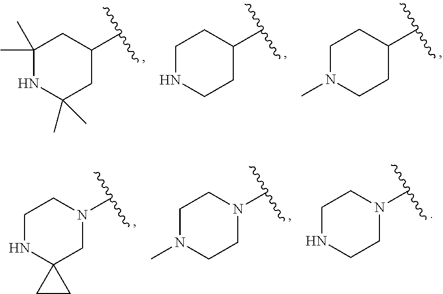

In some embodiments, B is

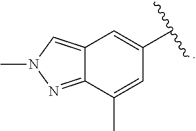 [SMILES: *c1cc(C)c2nn(C)cc2c1]

In some embodiments, B is

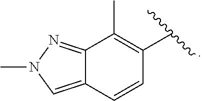 [SMILES: *c1ccc2cn(C)nc2c1C]

In some embodiments, B is

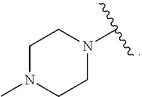 [SMILES: *N1CCN(C)CC1]

In some embodiments, B is

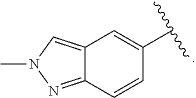 [SMILES: *c1ccc2nn(C)cc2c1]

In some embodiments, B is

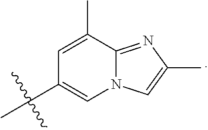 [SMILES: *c1cc(C)c2nc(C)cn2c1]

In some embodiments, B is

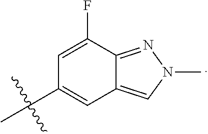 [SMILES: *c1cc(F)c2nn(C)cc2c1]

In some embodiments, B is

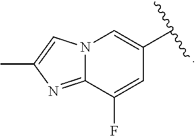 [SMILES: *c1cc(F)c2nc(C)cn2c1]

In some embodiments, B is

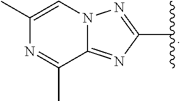 [SMILES: *c1nc2c(C)nc(C)cn2n1]

In some embodiments, B is

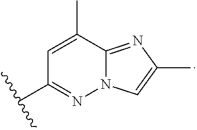 [SMILES: *c1cc(C)c2nc(C)cn2n1]

In some embodiments, B is

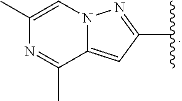 [SMILES: *c1cc2c(C)nc(C)cn2n1]

In some embodiments, B is

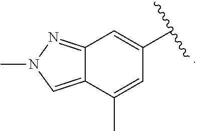 [SMILES: *c1cc(C)c2cn(C)nc2c1]

In some embodiments, B is

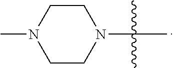 [SMILES: *N1CCN(C)CC1]

In some embodiments, B is

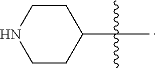 [SMILES: *C1CCNCC1]

In some embodiments, B is

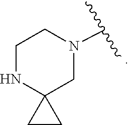 [SMILES: *N1CCNC2(CC2)C1]

In some embodiments, the compound of Formula (I) is selected from a compound in Table 1, or a pharmaceutically acceptable salt, solvate, hydrate, tautomer, or stereoisomer thereof.

TABLE 1
Exemplary compounds of Formula (I)

| Cmpd | |
|---|---|
| No. | Structure |
| 185 | 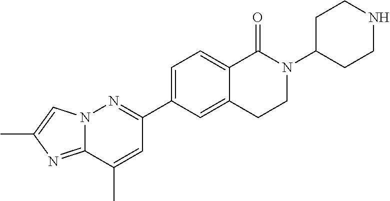 [SMILES: Cc1cn2nc(-c3ccc4c(c3)CCN(C3CCNCC3)C4=O)cc(C)c2n1] |
| 186 | 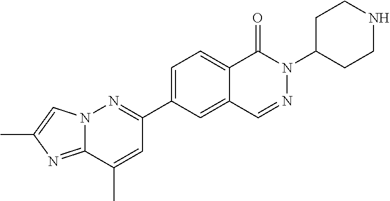 [SMILES: Cc1cn2nc(-c3ccc4c(=O)n(C5CCNCC5)ncc4c3)cc(C)c2n1] |
| 187 | 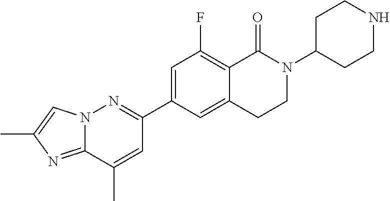 [SMILES: Cc1cn2nc(-c3cc(F)c4c(c3)CCN(C3CCNCC3)C4=O)cc(C)c2n1] |
| 188 | 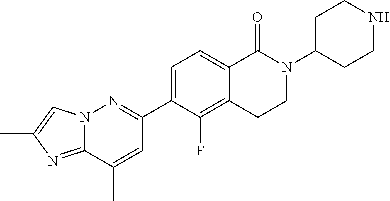 [SMILES: Cc1cn2nc(-c3ccc4c(c3F)CCN(C3CCNCC3)C4=O)cc(C)c2n1] |
| 215 | 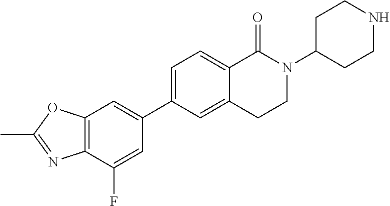 [SMILES: Cc1nc2c(F)cc(-c3ccc4c(c3)CCN(C3CCNCC3)C4=O)cc2o1] |
| 216 | 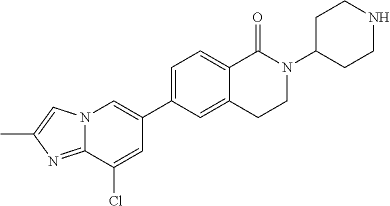 [SMILES: Cc1cn2cc(-c3ccc4c(c3)CCN(C3CCNCC3)C4=O)cc(Cl)c2n1] |
| 217 | 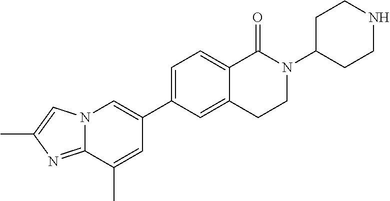 [SMILES: Cc1cn2cc(-c3ccc4c(c3)CCN(C3CCNCC3)C4=O)cc(C)c2n1] |
| 218 | 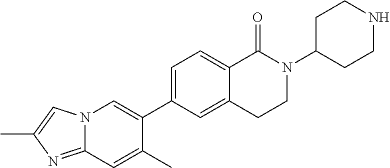 [SMILES: Cc1cn2cc(-c3ccc4c(c3)CCN(C3CCNCC3)C4=O)c(C)cc2n1] |
| 219 | 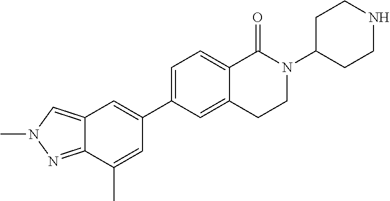 [SMILES: Cc1cc(-c2ccc3c(c2)CCN(C2CCNCC2)C3=O)cc2cn(C)nc12] |
| 220 | 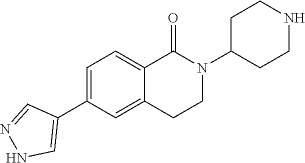 |
| 221 | 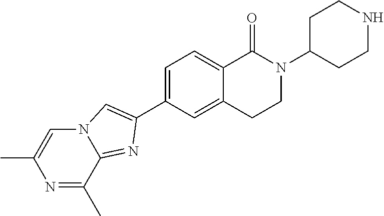 [SMILES: Cc1cn2cc(-c3ccc4c(c3)CCN(C3CCNCC3)C4=O)nc2c(C)n1] |
| 222 | 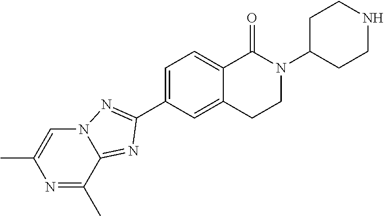 [SMILES: Cc1cn2nc(-c3ccc4c(c3)CCN(C3CCNCC3)C4=O)nc2c(C)n1] |
| 223 | 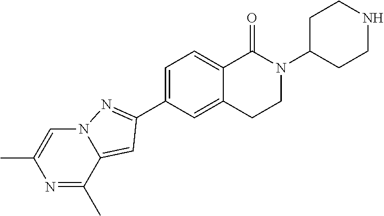 [SMILES: Cc1cn2nc(-c3ccc4c(c3)CCN(C3CCNCC3)C4=O)cc2c(C)n1] |
| 224 | 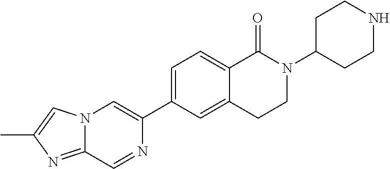 [SMILES: Cc1cn2cc(-c3ccc4c(c3)CCN(C3CCNCC3)C4=O)ncc2n1] |
| 225 | 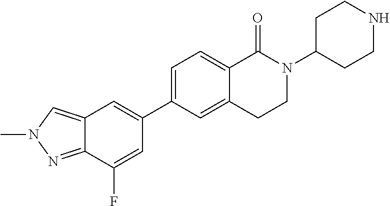 [SMILES: Cn1cc2cc(-c3ccc4c(c3)CCN(C3CCNCC3)C4=O)cc(F)c2n1] |
| 226 | 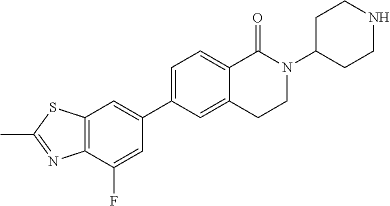 [SMILES: Cc1nc2c(F)cc(-c3ccc4c(c3)CCN(C3CCNCC3)C4=O)cc2s1] |
| 247 | 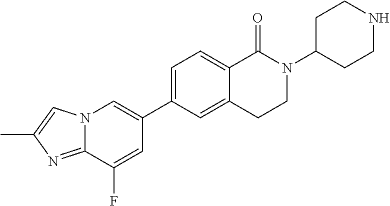 [SMILES: Cc1cn2cc(-c3ccc4c(c3)CCN(C3CCNCC3)C4=O)cc(F)c2n1] |
| 248 | 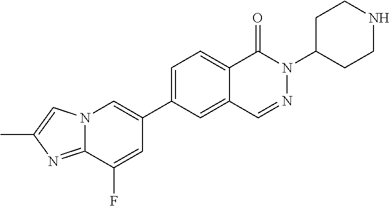 [SMILES: Cc1cn2cc(-c3ccc4c(=O)n(C5CCNCC5)ncc4c3)cc(F)c2n1] |
| 249 | 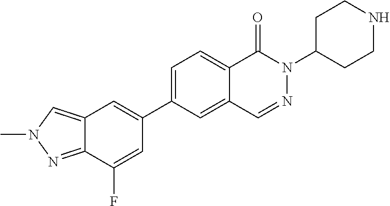 [SMILES: Cn1cc2cc(-c3ccc4c(=O)n(C5CCNCC5)ncc4c3)cc(F)c2n1] |
| 250 | 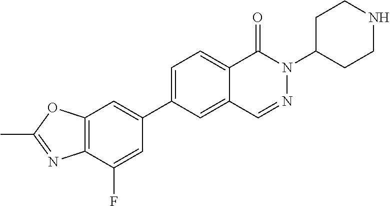 [SMILES: Cc1nc2c(F)cc(-c3ccc4c(=O)n(C5CCNCC5)ncc4c3)cc2o1] |
| 251 | 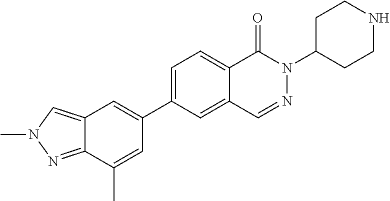 [SMILES: Cc1cc(-c2ccc3c(=O)n(C4CCNCC4)ncc3c2)cc2cn(C)nc12] |
| 252 | 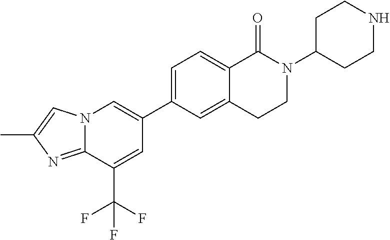 [SMILES: Cc1cn2cc(-c3ccc4c(c3)CCN(C3CCNCC3)C4=O)cc(C(F)(F)F)c2n1] |
| 253 | 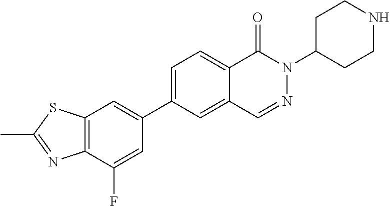 [SMILES: Cc1nc2c(F)cc(-c3ccc4c(=O)n(C5CCNCC5)ncc4c3)cc2s1] |
| 254 | 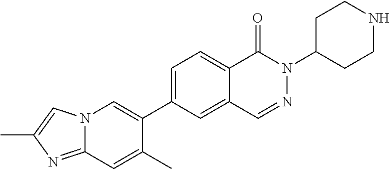 [SMILES: Cc1cn2cc(-c3ccc4c(=O)n(C5CCNCC5)ncc4c3)c(C)cc2n1] |
| 255 | 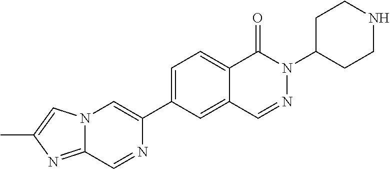 [SMILES: Cc1cn2cc(-c3ccc4c(=O)n(C5CCNCC5)ncc4c3)ncc2n1] |
| 256 | 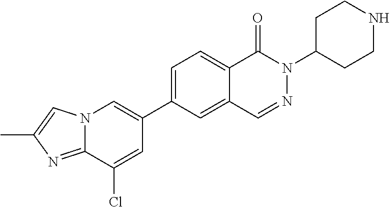 [SMILES: Cc1cn2cc(-c3ccc4c(=O)n(C5CCNCC5)ncc4c3)cc(Cl)c2n1] |
| 257 | 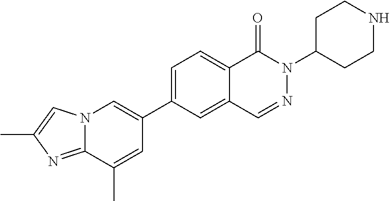 [SMILES: Cc1cn2cc(-c3ccc4c(=O)n(C5CCNCC5)ncc4c3)cc(C)c2n1] |
| 258 | 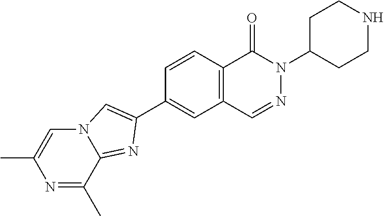 [SMILES: Cc1cn2cc(-c3ccc4c(=O)n(C5CCNCC5)ncc4c3)nc2c(C)n1] |
| 259 | 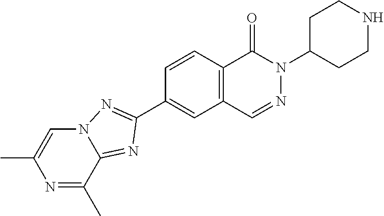 [SMILES: Cc1cn2nc(-c3ccc4c(=O)n(C5CCNCC5)ncc4c3)nc2c(C)n1] |
| 260 | 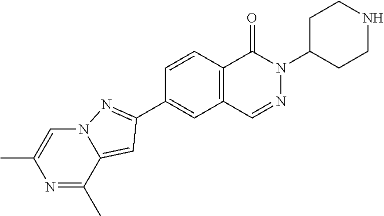 [SMILES: Cc1cn2nc(-c3ccc4c(=O)n(C5CCNCC5)ncc4c3)cc2c(C)n1] |
| 261 | 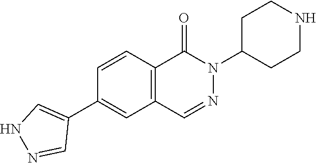 |
| 262 | 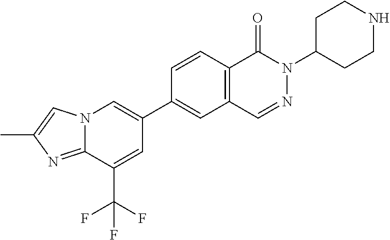 [SMILES: Cc1cn2cc(-c3ccc4c(=O)n(C5CCNCC5)ncc4c3)cc(C(F)(F)F)c2n1] |
| 263 | 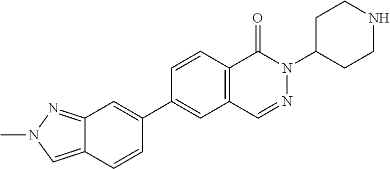 [SMILES: Cn1cc2ccc(-c3ccc4c(=O)n(C5CCNCC5)ncc4c3)cc2n1] |
| 264 | 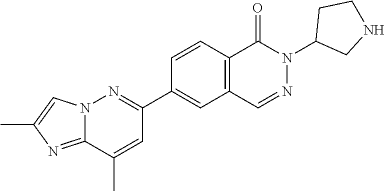 [SMILES: Cc1cn2nc(-c3ccc4c(=O)n(C5CCNC5)ncc4c3)cc(C)c2n1] |
| 265 | 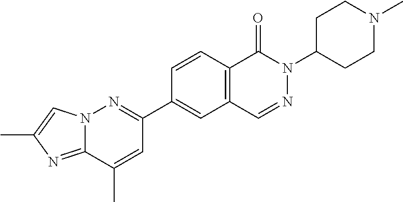 |
| 266 | 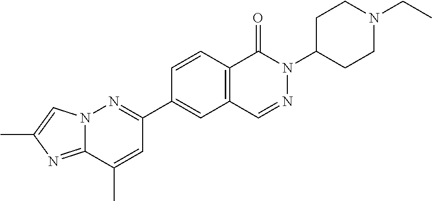 [SMILES: CCN1CCC(n2ncc3cc(-c4cc(C)c5nc(C)cn5n4)ccc3c2=O)CC1] |
| 267 | 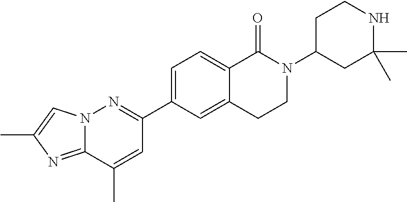 |
| 268 | 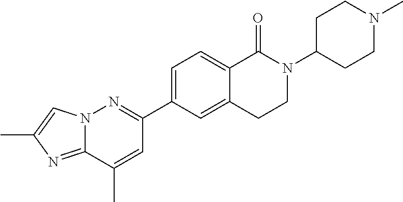 |
| 269 | 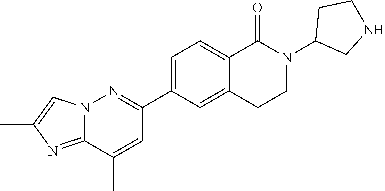 [SMILES: Cc1cn2nc(-c3ccc4c(c3)CCN(C3CCNC3)C4=O)cc(C)c2n1] |
| 270 | 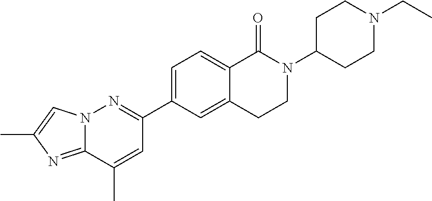 [SMILES: CCN1CCC(N2CCc3cc(-c4cc(C)c5nc(C)cn5n4)ccc3C2=O)CC1] |
| 271 | 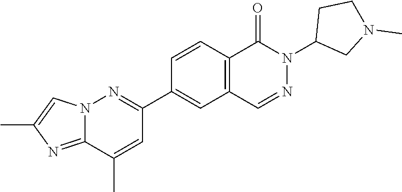 |
| 272 | 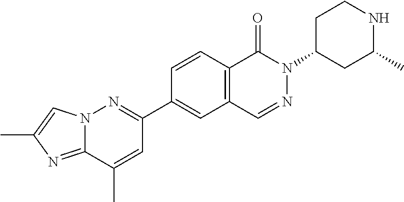 |
| 273 | 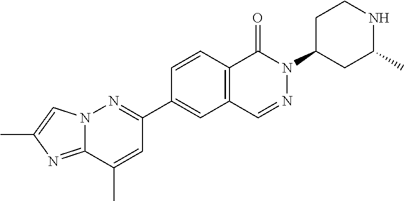 |
| 274 | 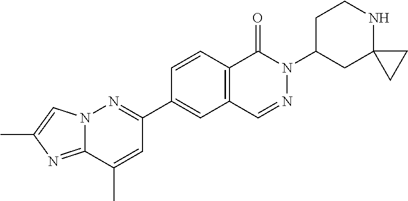 [SMILES: Cc1cn2nc(-c3ccc4c(=O)n(C5CCNC6(CC6)C5)ncc4c3)cc(C)c2n1] |
| 275 | 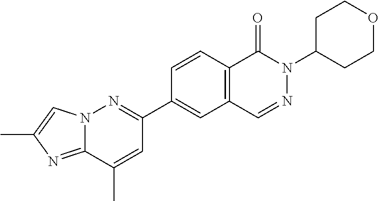 [SMILES: Cc1cn2nc(-c3ccc4c(=O)n(C5CCOCC5)ncc4c3)cc(C)c2n1] |
| 276 | 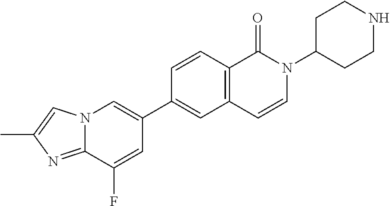 [SMILES: Cc1cn2cc(-c3ccc4c(=O)n(C5CCNCC5)ccc4c3)cc(F)c2n1] |
| 277 | 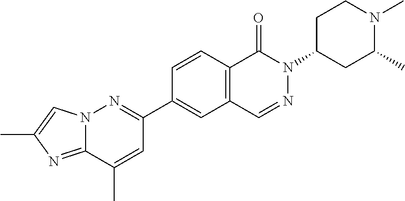 |
| 278 | 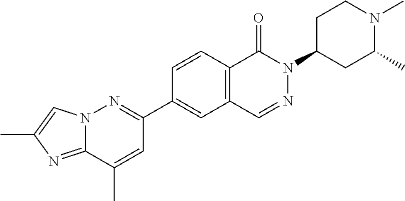 |
| 279 | 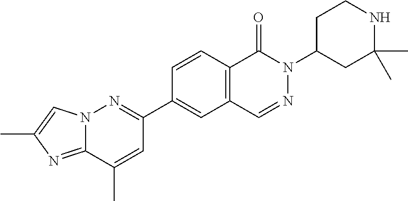 |
| 280 | 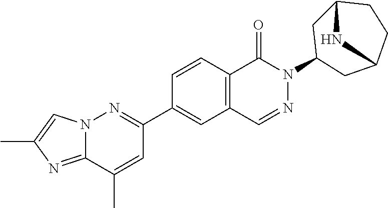 [SMILES: Cc1cn2nc(-c3ccc4c(=O)n([C@@H]5C[C@H]6CC[C@@H](C5)N6)ncc4c3)cc(C)c2n1] |
| 281 | 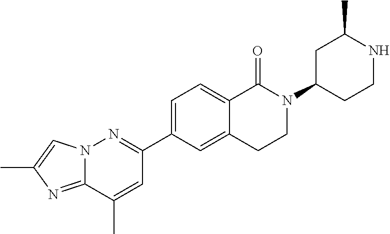 [SMILES: Cc1cn2nc(-c3ccc4c(c3)CCN([C@@H]3CCN[C@H](C)C3)C4=O)cc(C)c2n1] |
| 282 | 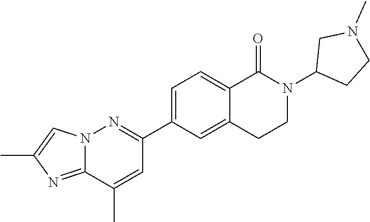 [SMILES: Cc1cn2nc(-c3ccc4c(c3)CCN(C3CCN(C)C3)C4=O)cc(C)c2n1] |
| 283 | 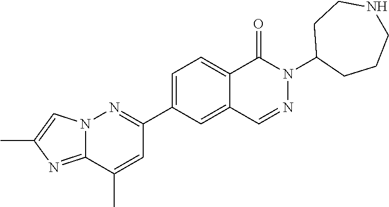 [SMILES: Cc1cn2nc(-c3ccc4c(=O)n(C5CCCNCC5)ncc4c3)cc(C)c2n1] |
| 304 | 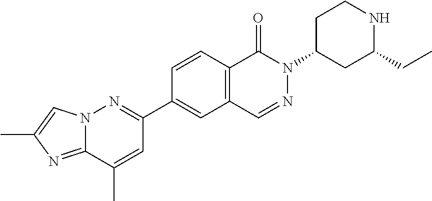 [SMILES: CC[C@@H]1C[C@H](n2ncc3cc(-c4cc(C)c5nc(C)cn5n4)ccc3c2=O)CCN1] |
| 305 | 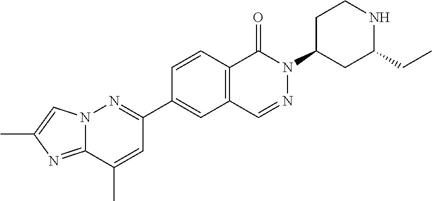 [SMILES: CC[C@@H]1C[C@@H](n2ncc3cc(-c4cc(C)c5nc(C)cn5n4)ccc3c2=O)CCN1] |
| 309 | 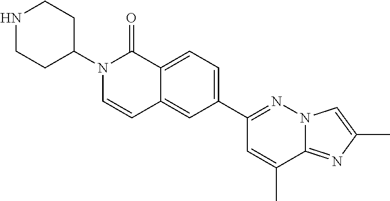 [SMILES: Cc1cn2nc(-c3ccc4c(=O)n(C5CCNCC5)ccc4c3)cc(C)c2n1] |
| 310 | 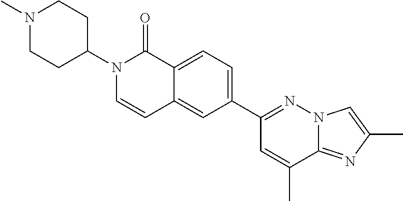 |
| 312 | 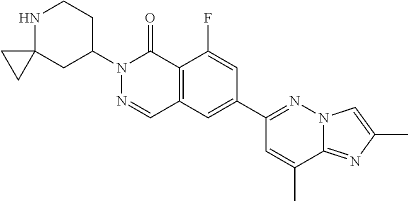 [SMILES: Cc1cn2nc(-c3cc(F)c4c(=O)n(C5CCNC6(CC6)C5)ncc4c3)cc(C)c2n1] |
| 322 | 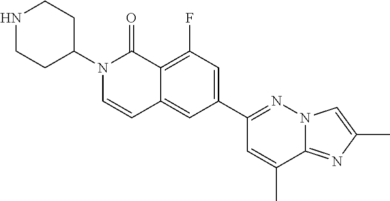 [SMILES: Cc1cn2nc(-c3cc(F)c4c(=O)n(C5CCNCC5)ccc4c3)cc(C)c2n1] |
| 324 | 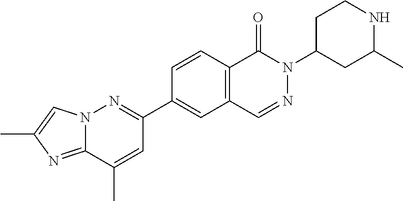 |
| 325 | 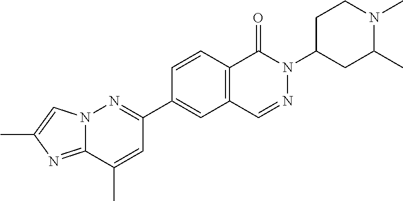 |
| 326 | 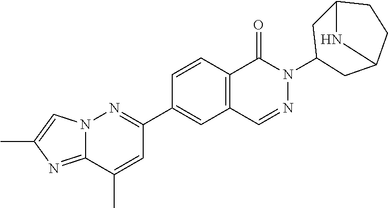 [SMILES: Cc1cn2nc(-c3ccc4c(=O)n(C5CC6CCC(C5)N6)ncc4c3)cc(C)c2n1] |
| 327 | 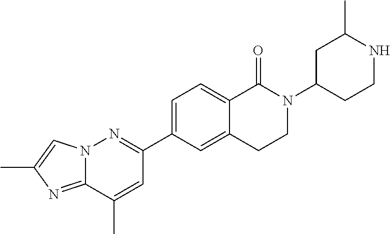 [SMILES: Cc1cn2nc(-c3ccc4c(c3)CCN(C3CCNC(C)C3)C4=O)cc(C)c2n1] |
| 328 | 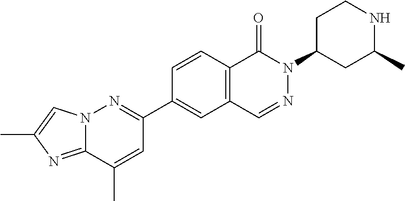 |
| 329 | 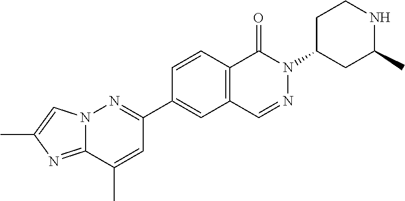 |
| 330 | 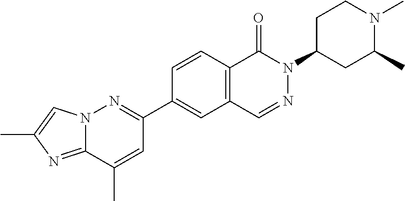 |
| 331 | 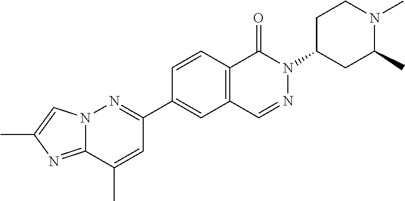 |
| 332 | 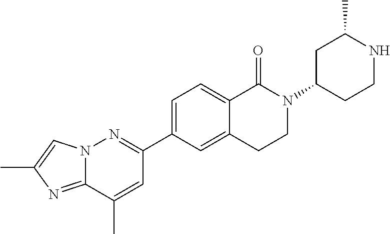 [SMILES: Cc1cn2nc(-c3ccc4c(c3)CCN([C@H]3CCN[C@@H](C)C3)C4=O)cc(C)c2n1] |
| 333 | 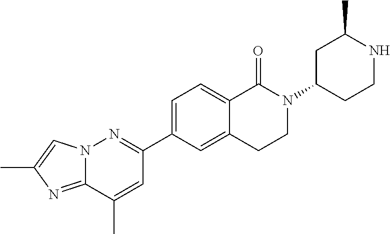 [SMILES: Cc1cn2nc(-c3ccc4c(c3)CCN([C@H]3CCN[C@H](C)C3)C4=O)cc(C)c2n1] |
| 334 | 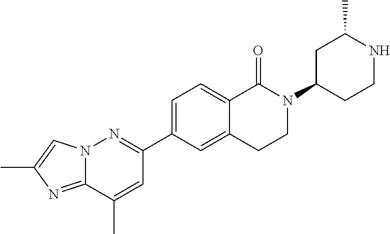 [SMILES: Cc1cn2nc(-c3ccc4c(c3)CCN([C@@H]3CCN[C@@H](C)C3)C4=O)cc(C)c2n1] |
| 335 | 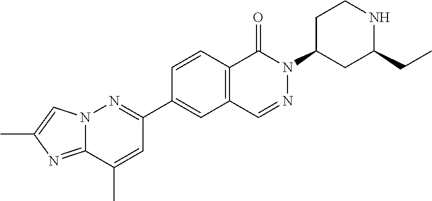 [SMILES: CC[C@H]1C[C@@H](n2ncc3cc(-c4cc(C)c5nc(C)cn5n4)ccc3c2=O)CCN1] |
| 336 | 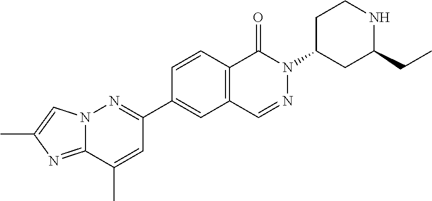 [SMILES: CC[C@H]1C[C@H](n2ncc3cc(-c4cc(C)c5nc(C)cn5n4)ccc3c2=O)CCN1] |
| 368 | 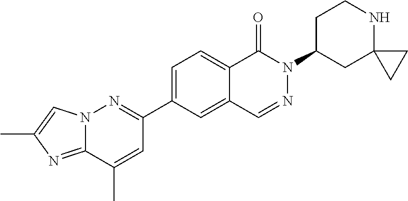 [SMILES: Cc1cn2nc(-c3ccc4c(=O)n([C@H]5CCNC6(CC6)C5)ncc4c3)cc(C)c2n1] |
| 369 | 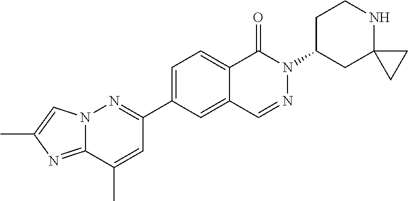 [SMILES: Cc1cn2nc(-c3ccc4c(=O)n([C@@H]5CCNC6(CC6)C5)ncc4c3)cc(C)c2n1] |
| 370 | 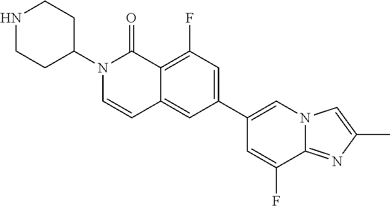 [SMILES: Cc1cn2cc(-c3cc(F)c4c(=O)n(C5CCNCC5)ccc4c3)cc(F)c2n1] |
| 372 | 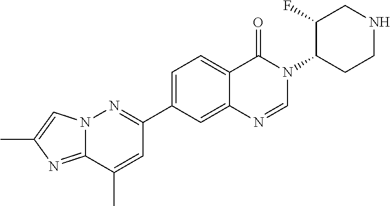 [SMILES: Cc1cn2nc(-c3ccc4c(=O)n([C@H]5CCNC[C@H]5F)cnc4c3)cc(C)c2n1] |
| 375 | 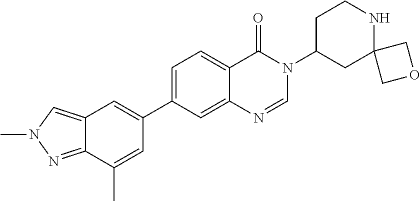 [SMILES: Cc1cc(-c2ccc3c(=O)n(C4CCNC5(COC5)C4)cnc3c2)cc2cn(C)nc12] |
| 378 | 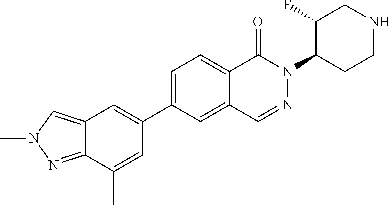 [SMILES: Cc1cc(-c2ccc3c(=O)n([C@@H]4CCNC[C@H]4F)ncc3c2)cc2cn(C)nc12] |
| 379 | 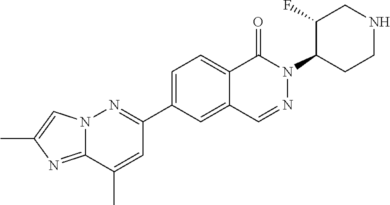 [SMILES: Cc1cn2nc(-c3ccc4c(=O)n([C@@H]5CCNC[C@H]5F)ncc4c3)cc(C)c2n1] |
| 380 | 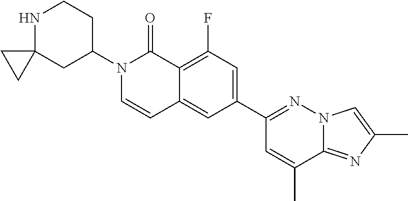 [SMILES: Cc1cn2nc(-c3cc(F)c4c(=O)n(C5CCNC6(CC6)C5)ccc4c3)cc(C)c2n1] |
| 381 | 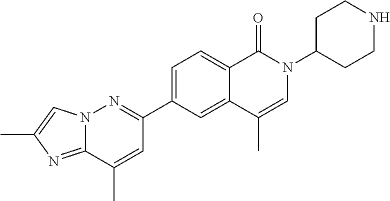 [SMILES: Cc1cn2nc(-c3ccc4c(=O)n(C5CCNCC5)cc(C)c4c3)cc(C)c2n1] |
| 382 | 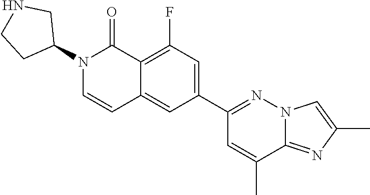 [SMILES: Cc1cn2nc(-c3cc(F)c4c(=O)n([C@H]5CCNC5)ccc4c3)cc(C)c2n1] |
| 383 | 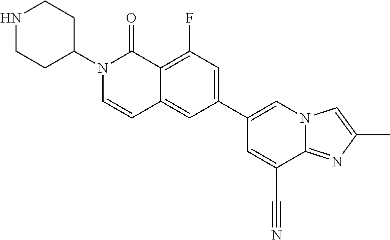 [SMILES: Cc1cn2cc(-c3cc(F)c4c(=O)n(C5CCNCC5)ccc4c3)cc(C#N)c2n1] |
| 384 | 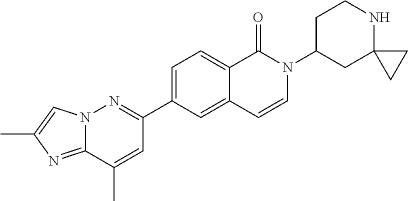 [SMILES: Cc1cn2nc(-c3ccc4c(=O)n(C5CCNC6(CC6)C5)ccc4c3)cc(C)c2n1] |
| 387 | 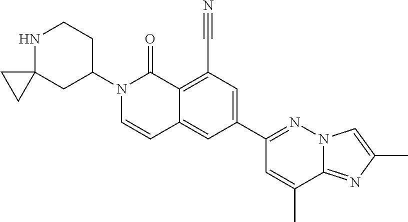 [SMILES: Cc1cn2nc(-c3cc(C#N)c4c(=O)n(C5CCNC6(CC6)C5)ccc4c3)cc(C)c2n1] |
| 390 | 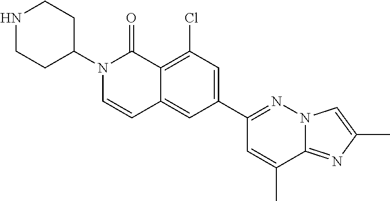 [SMILES: Cc1cn2nc(-c3cc(Cl)c4c(=O)n(C5CCNCC5)ccc4c3)cc(C)c2n1] |
| 391 | 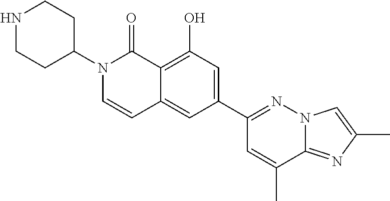 [SMILES: Cc1cn2nc(-c3cc(O)c4c(=O)n(C5CCNCC5)ccc4c3)cc(C)c2n1] |
| 392 | 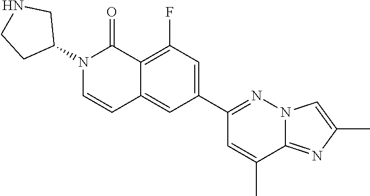 [SMILES: Cc1cn2nc(-c3cc(F)c4c(=O)n([C@@H]5CCNC5)ccc4c3)cc(C)c2n1] |
| 393 | 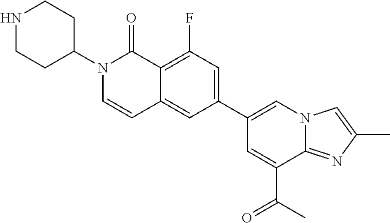 [SMILES: CC(=O)c1cc(-c2cc(F)c3c(=O)n(C4CCNCC4)ccc3c2)cn2cc(C)nc12] |
| 394 | 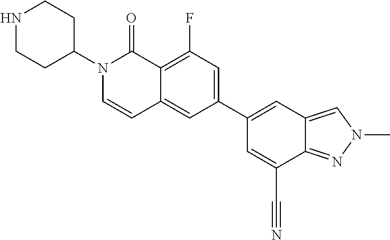 [SMILES: Cn1cc2cc(-c3cc(F)c4c(=O)n(C5CCNCC5)ccc4c3)cc(C#N)c2n1] |
| 395 | 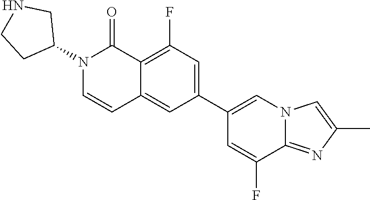 [SMILES: Cc1cn2cc(-c3cc(F)c4c(=O)n([C@@H]5CCNC5)ccc4c3)cc(F)c2n1] |
| 396 | 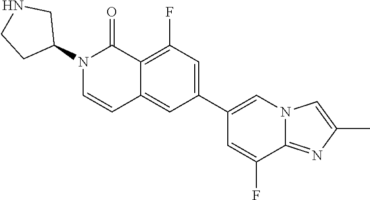 [SMILES: Cc1cn2cc(-c3cc(F)c4c(=O)n([C@H]5CCNC5)ccc4c3)cc(F)c2n1] |
| 397 | 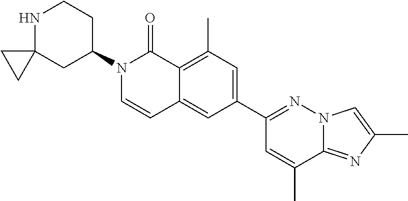 [SMILES: Cc1cn2nc(-c3cc(C)c4c(=O)n([C@@H]5CCNC6(CC6)C5)ccc4c3)cc(C)c2n1] |
| 398 | 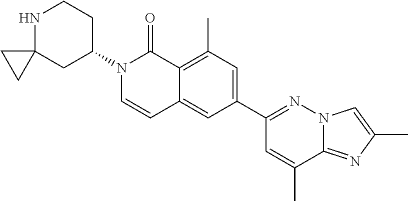 [SMILES: Cc1cn2nc(-c3cc(C)c4c(=O)n([C@H]5CCNC6(CC6)C5)ccc4c3)cc(C)c2n1] |
| 399 | 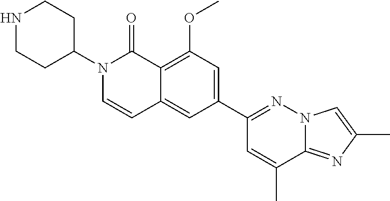 [SMILES: COc1cc(-c2cc(C)c3nc(C)cn3n2)cc2ccn(C3CCNCC3)c(=O)c12] |
| 400 | 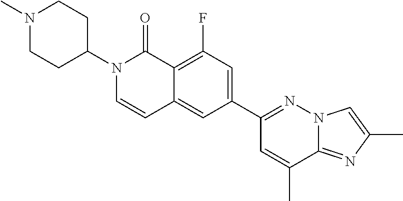 |
| 401 | 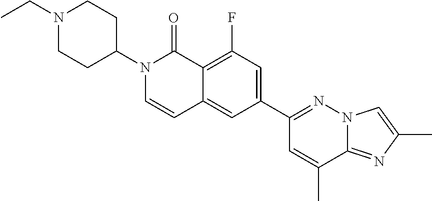 [SMILES: CCN1CCC(n2ccc3cc(-c4cc(C)c5nc(C)cn5n4)cc(F)c3c2=O)CC1] |
| 402 | 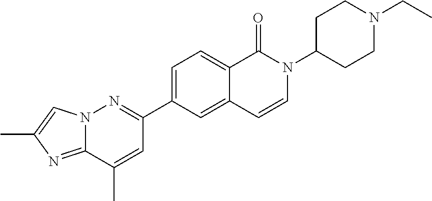 [SMILES: CCN1CCC(n2ccc3cc(-c4cc(C)c5nc(C)cn5n4)ccc3c2=O)CC1] |
| 403 | 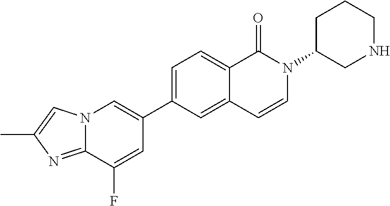 [SMILES: Cc1cn2cc(-c3ccc4c(=O)n([C@@H]5CCCNC5)ccc4c3)cc(F)c2n1] |
| 404 | 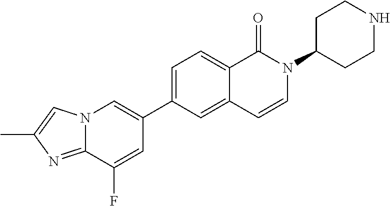 |
| 409 | 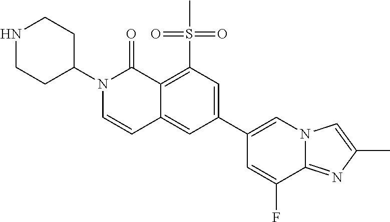 [SMILES: Cc1cn2cc(-c3cc(S(C)(=O)=O)c4c(=O)n(C5CCNCC5)ccc4c3)cc(F)c2n1] |
| 410 | 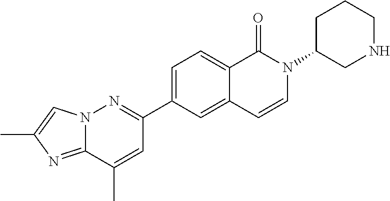 [SMILES: Cc1cn2nc(-c3ccc4c(=O)n([C@@H]5CCCNC5)ccc4c3)cc(C)c2n1] |
| 411 | 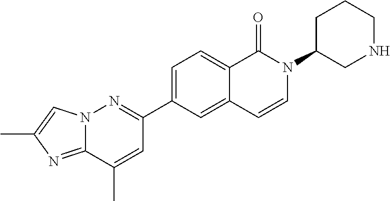 [SMILES: Cc1cn2nc(-c3ccc4c(=O)n([C@H]5CCCNC5)ccc4c3)cc(C)c2n1] |
| 412 | 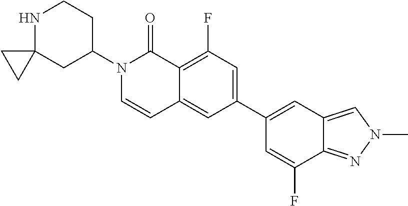 [SMILES: Cn1cc2cc(-c3cc(F)c4c(=O)n(C5CCNC6(CC6)C5)ccc4c3)cc(F)c2n1] |
| 413 | 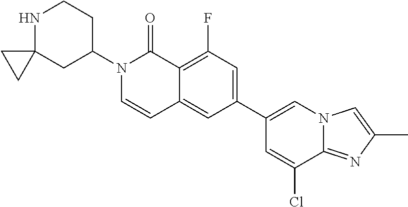 [SMILES: Cc1cn2cc(-c3cc(F)c4c(=O)n(C5CCNC6(CC6)C5)ccc4c3)cc(Cl)c2n1] |
| 414 | 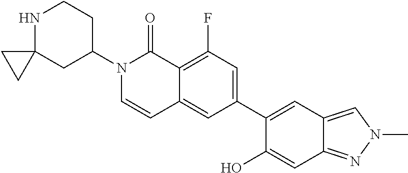 [SMILES: Cn1cc2cc(-c3cc(F)c4c(=O)n(C5CCNC6(CC6)C5)ccc4c3)c(O)cc2n1] |
| 415 | 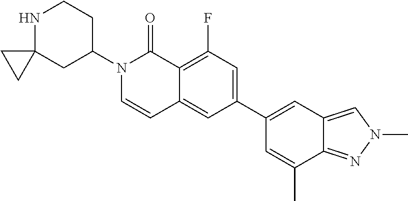 [SMILES: Cc1cc(-c2cc(F)c3c(=O)n(C4CCNC5(CC5)C4)ccc3c2)cc2cn(C)nc12] |
| 416 | 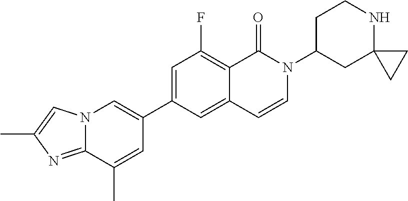 [SMILES: Cc1cn2cc(-c3cc(F)c4c(=O)n(C5CCNC6(CC6)C5)ccc4c3)cc(C)c2n1] |
| 417 | 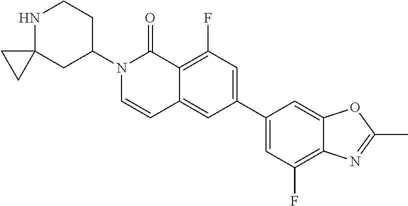 [SMILES: Cc1nc2c(F)cc(-c3cc(F)c4c(=O)n(C5CCNC6(CC6)C5)ccc4c3)cc2o1] |
| 418 | 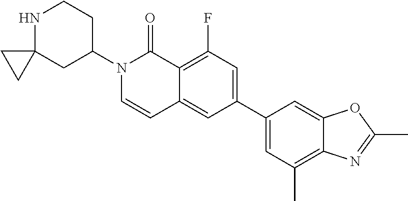 [SMILES: Cc1nc2c(C)cc(-c3cc(F)c4c(=O)n(C5CCNC6(CC6)C5)ccc4c3)cc2o1] |
| 419 | 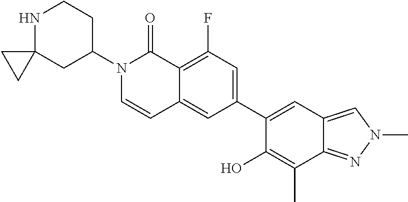 [SMILES: Cc1c(O)c(-c2cc(F)c3c(=O)n(C4CCNC5(CC5)C4)ccc3c2)cc2cn(C)nc12] |
| 420 | 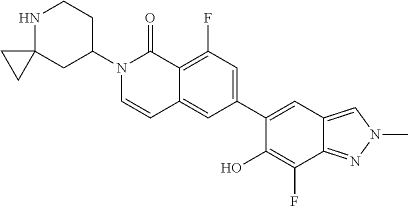 [SMILES: Cn1cc2cc(-c3cc(F)c4c(=O)n(C5CCNC6(CC6)C5)ccc4c3)c(O)c(F)c2n1] |
| 421 | 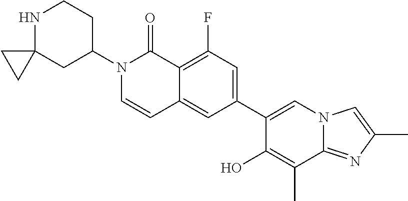 [SMILES: Cc1cn2cc(-c3cc(F)c4c(=O)n(C5CCNC6(CC6)C5)ccc4c3)c(O)c(C)c2n1] |
| 423 | 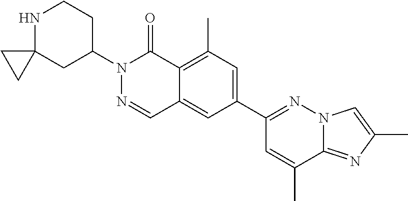 [SMILES: Cc1cn2nc(-c3cc(C)c4c(=O)n(C5CCNC6(CC6)C5)ncc4c3)cc(C)c2n1] |
| 425 | 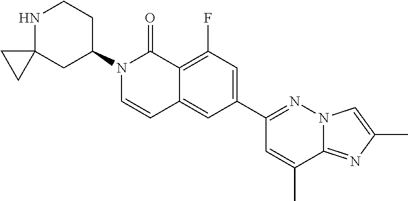 [SMILES: Cc1cn2nc(-c3cc(F)c4c(=O)n([C@@H]5CCNC6(CC6)C5)ccc4c3)cc(C)c2n1] |
| 426 | 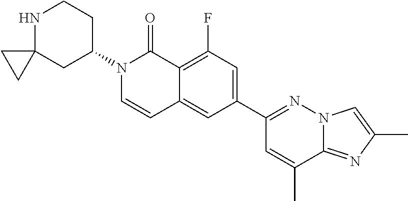 [SMILES: Cc1cn2nc(-c3cc(F)c4c(=O)n([C@H]5CCNC6(CC6)C5)ccc4c3)cc(C)c2n1] |
| 428 | 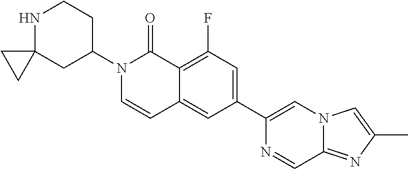 [SMILES: Cc1cn2cc(-c3cc(F)c4c(=O)n(C5CCNC6(CC6)C5)ccc4c3)ncc2n1] |
| 429 | 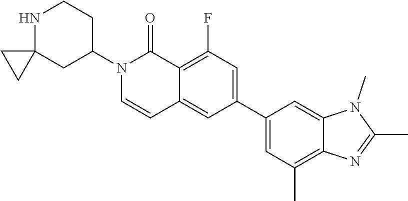 [SMILES: Cc1cc(-c2cc(F)c3c(=O)n(C4CCNC5(CC5)C4)ccc3c2)cc2c1nc(C)n2C] |
| 430 | 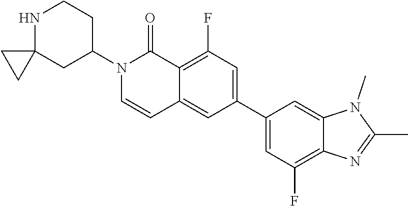 [SMILES: Cc1nc2c(F)cc(-c3cc(F)c4c(=O)n(C5CCNC6(CC6)C5)ccc4c3)cc2n1C] |
| 431 | 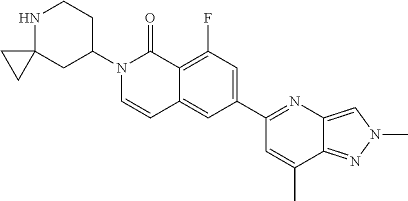 [SMILES: Cc1cc(-c2cc(F)c3c(=O)n(C4CCNC5(CC5)C4)ccc3c2)nc2cn(C)nc12] |
| 432 | 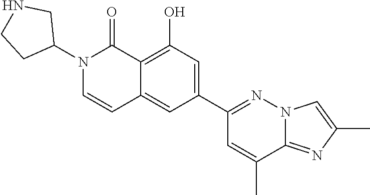 [SMILES: Cc1cn2nc(-c3cc(O)c4c(=O)n(C5CCNC5)ccc4c3)cc(C)c2n1] |
| 433 | 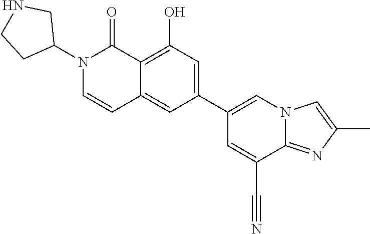 [SMILES: Cc1cn2cc(-c3cc(O)c4c(=O)n(C5CCNC5)ccc4c3)cc(C#N)c2n1] |
| 434 | 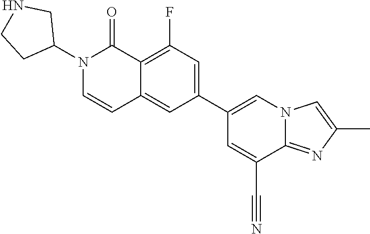 [SMILES: Cc1cn2cc(-c3cc(F)c4c(=O)n(C5CCNC5)ccc4c3)cc(C#N)c2n1] |
| 440 | 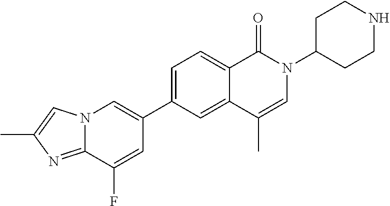 [SMILES: Cc1cn2cc(-c3ccc4c(=O)n(C5CCNCC5)cc(C)c4c3)cc(F)c2n1] |
| 441 | 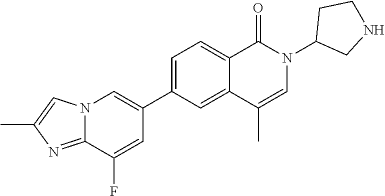 [SMILES: Cc1cn2cc(-c3ccc4c(=O)n(C5CCNC5)cc(C)c4c3)cc(F)c2n1] |
| 442 | 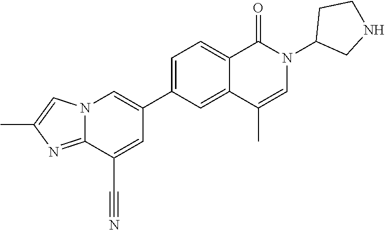 [SMILES: Cc1cn2cc(-c3ccc4c(=O)n(C5CCNC5)cc(C)c4c3)cc(C#N)c2n1] |
| 443 | 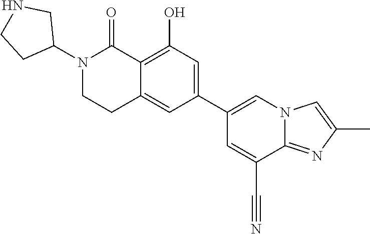 [SMILES: Cc1cn2cc(-c3cc(O)c4c(c3)CCN(C3CCNC3)C4=O)cc(C#N)c2n1] |
| 444 | 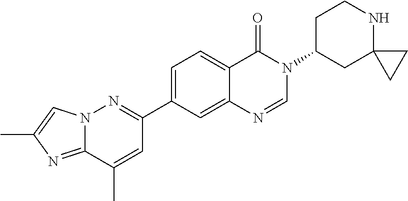 [SMILES: Cc1cn2nc(-c3ccc4c(=O)n([C@@H]5CCNC6(CC6)C5)cnc4c3)cc(C)c2n1] |
| 445 | 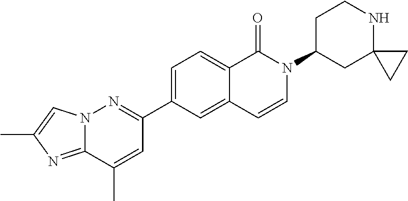 [SMILES: Cc1cn2nc(-c3ccc4c(=O)n([C@H]5CCNC6(CC6)C5)ccc4c3)cc(C)c2n1] |
| 446 | 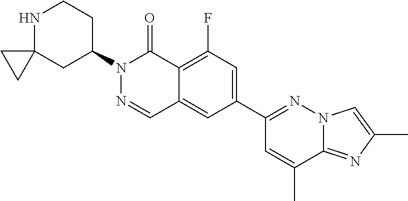 [SMILES: Cc1cn2nc(-c3cc(F)c4c(=O)n([C@@H]5CCNC6(CC6)C5)ncc4c3)cc(C)c2n1] |
| 447 | 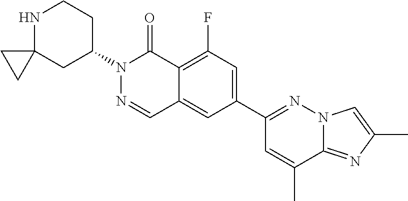 [SMILES: Cc1cn2nc(-c3cc(F)c4c(=O)n([C@H]5CCNC6(CC6)C5)ncc4c3)cc(C)c2n1] |
| 448 | 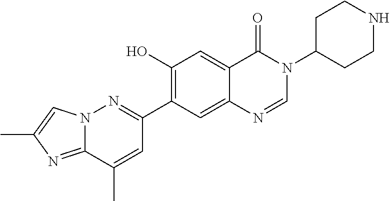 [SMILES: Cc1cn2nc(-c3cc4ncn(C5CCNCC5)c(=O)c4cc3O)cc(C)c2n1] |
| 449 | 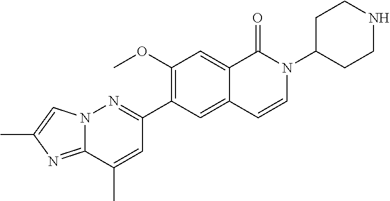 [SMILES: COc1cc2c(=O)n(C3CCNCC3)ccc2cc1-c1cc(C)c2nc(C)cn2n1] |
| 451 | 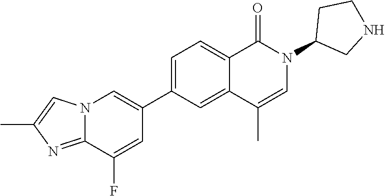 [SMILES: Cc1cn2cc(-c3ccc4c(=O)n([C@H]5CCNC5)cc(C)c4c3)cc(F)c2n1] |
| 452 | 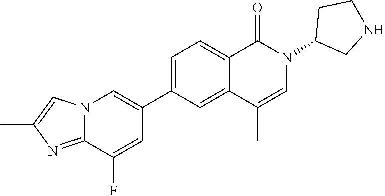 [SMILES: Cc1cn2cc(-c3ccc4c(=O)n([C@@H]5CCNC5)cc(C)c4c3)cc(F)c2n1] |
| 453 | 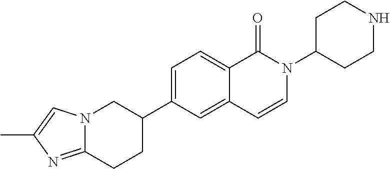 [SMILES: Cc1cn2c(n1)CCC(c1ccc3c(=O)n(C4CCNCC4)ccc3c1)C2] |
| 454 | 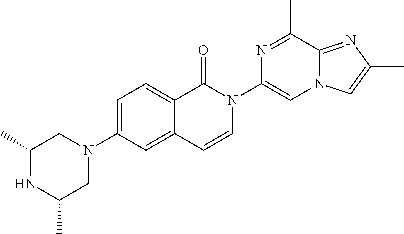 |
| 456 | 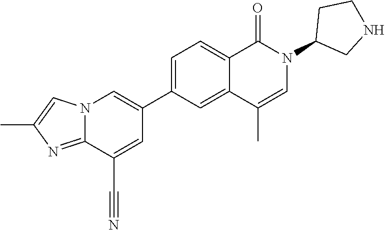 [SMILES: Cc1cn2cc(-c3ccc4c(=O)n([C@H]5CCNC5)cc(C)c4c3)cc(C#N)c2n1] |
| 457 | 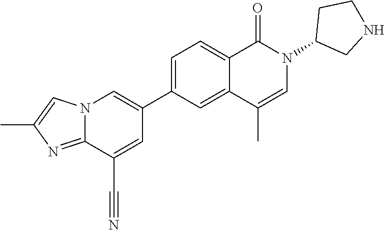 [SMILES: Cc1cn2cc(-c3ccc4c(=O)n([C@@H]5CCNC5)cc(C)c4c3)cc(C#N)c2n1] |
| 461 | 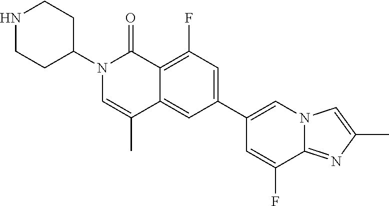 [SMILES: Cc1cn2cc(-c3cc(F)c4c(=O)n(C5CCNCC5)cc(C)c4c3)cc(F)c2n1] |
| 462 | 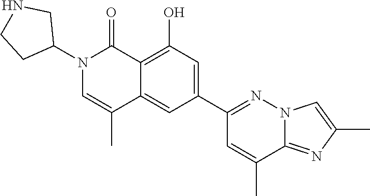 [SMILES: Cc1cn2nc(-c3cc(O)c4c(=O)n(C5CCNC5)cc(C)c4c3)cc(C)c2n1] |
| 466 | 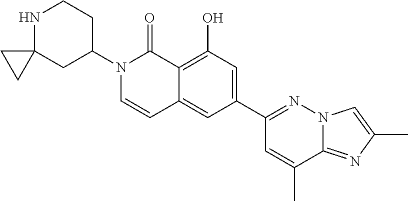 [SMILES: Cc1cn2nc(-c3cc(O)c4c(=O)n(C5CCNC6(CC6)C5)ccc4c3)cc(C)c2n1] |
| 467 | 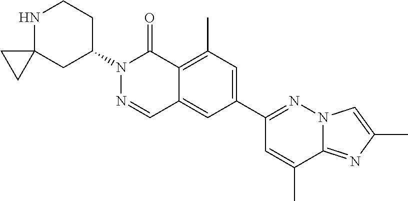 [SMILES: Cc1cn2nc(-c3cc(C)c4c(=O)n([C@H]5CCNC6(CC6)C5)ncc4c3)cc(C)c2n1] |
| 468 | 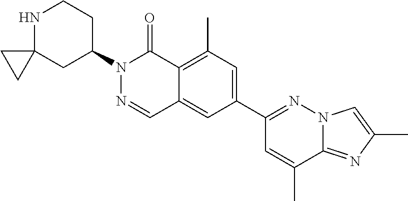 [SMILES: Cc1cn2nc(-c3cc(C)c4c(=O)n([C@@H]5CCNC6(CC6)C5)ncc4c3)cc(C)c2n1] |
| 469 | 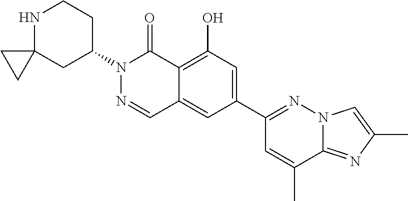 [SMILES: Cc1cn2nc(-c3cc(O)c4c(=O)n([C@H]5CCNC6(CC6)C5)ncc4c3)cc(C)c2n1] |
| 470 | 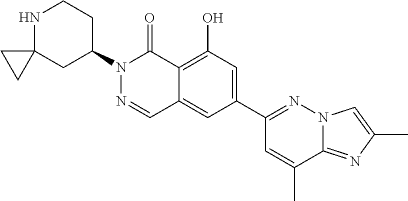 [SMILES: Cc1cn2nc(-c3cc(O)c4c(=O)n([C@@H]5CCNC6(CC6)C5)ncc4c3)cc(C)c2n1] |
| 471 | 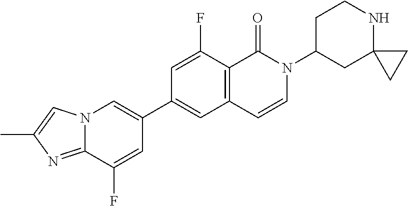 [SMILES: Cc1cn2cc(-c3cc(F)c4c(=O)n(C5CCNC6(CC6)C5)ccc4c3)cc(F)c2n1] |
| 472 | 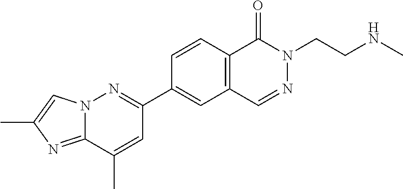 |
| 473 | 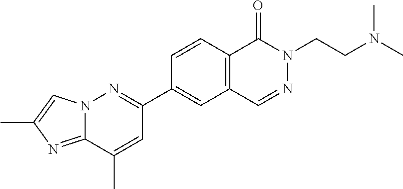 |
| 474 | 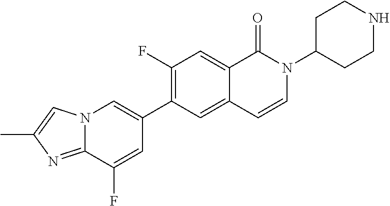 [SMILES: Cc1cn2cc(-c3cc4ccn(C5CCNCC5)c(=O)c4cc3F)cc(F)c2n1] |
| 475 | 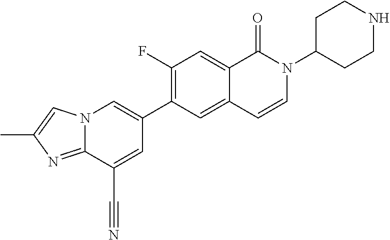 [SMILES: Cc1cn2cc(-c3cc4ccn(C5CCNCC5)c(=O)c4cc3F)cc(C#N)c2n1] |
| 476 | 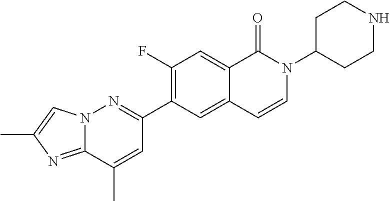 [SMILES: Cc1cn2nc(-c3cc4ccn(C5CCNCC5)c(=O)c4cc3F)cc(C)c2n1] |
| 477 | 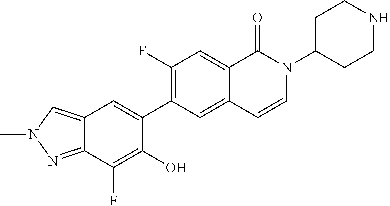 [SMILES: Cn1cc2cc(-c3cc4ccn(C5CCNCC5)c(=O)c4cc3F)c(O)c(F)c2n1] |
| 478 | 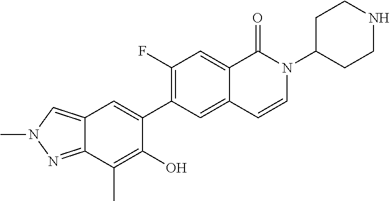 [SMILES: Cc1c(O)c(-c2cc3ccn(C4CCNCC4)c(=O)c3cc2F)cc2cn(C)nc12] |
| 479 | 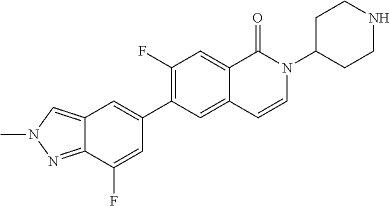 [SMILES: Cn1cc2cc(-c3cc4ccn(C5CCNCC5)c(=O)c4cc3F)cc(F)c2n1] |
| 480 | 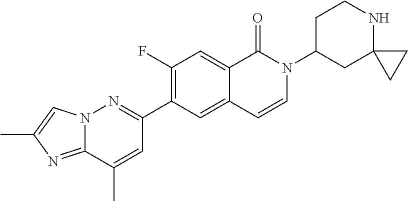 [SMILES: Cc1cn2nc(-c3cc4ccn(C5CCNC6(CC6)C5)c(=O)c4cc3F)cc(C)c2n1] |
| 481 | 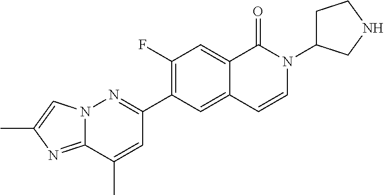 [SMILES: Cc1cn2nc(-c3cc4ccn(C5CCNC5)c(=O)c4cc3F)cc(C)c2n1] |
| 509 | 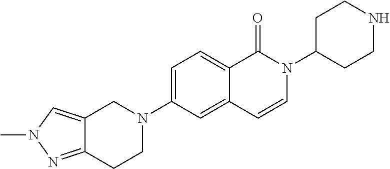 [SMILES: Cn1cc2c(n1)CCN(c1ccc3c(=O)n(C4CCNCC4)ccc3c1)C2] |
| 510 | 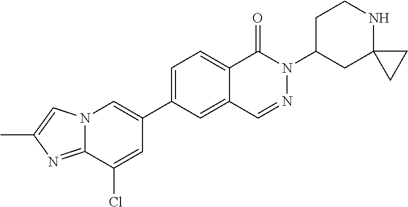 [SMILES: Cc1cn2cc(-c3ccc4c(=O)n(C5CCNC6(CC6)C5)ncc4c3)cc(Cl)c2n1] |
| 511 | 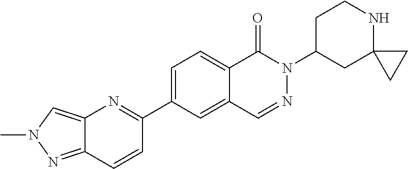 [SMILES: Cn1cc2nc(-c3ccc4c(=O)n(C5CCNC6(CC6)C5)ncc4c3)ccc2n1] |
| 512 | 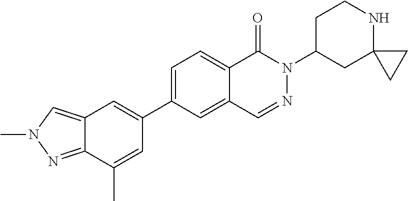 [SMILES: Cc1cc(-c2ccc3c(=O)n(C4CCNC5(CC5)C4)ncc3c2)cc2cn(C)nc12] |
| 513 | 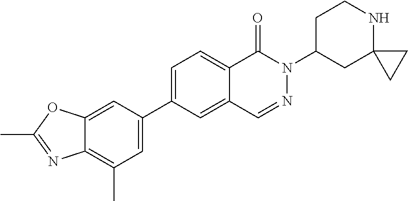 [SMILES: Cc1nc2c(C)cc(-c3ccc4c(=O)n(C5CCNC6(CC6)C5)ncc4c3)cc2o1] |
| 514 | 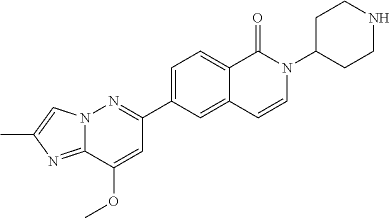 [SMILES: COc1cc(-c2ccc3c(=O)n(C4CCNCC4)ccc3c2)nn2cc(C)nc12] |
| 515 | 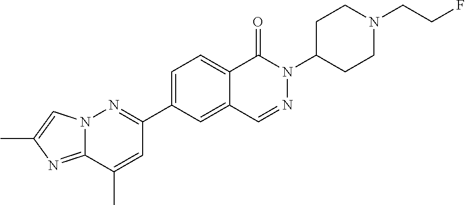 [SMILES: Cc1cn2nc(-c3ccc4c(=O)n(C5CCN(CCF)CC5)ncc4c3)cc(C)c2n1] |
| 516 | 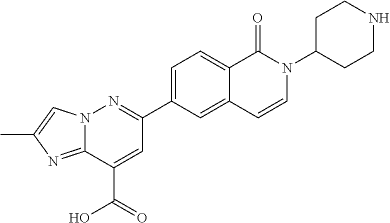 [SMILES: Cc1cn2nc(-c3ccc4c(=O)n(C5CCNCC5)ccc4c3)cc(C(=O)O)c2n1] |
| 517 | 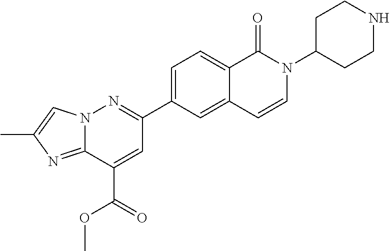 [SMILES: COC(=O)c1cc(-c2ccc3c(=O)n(C4CCNCC4)ccc3c2)nn2cc(C)nc12] |
| 518 | 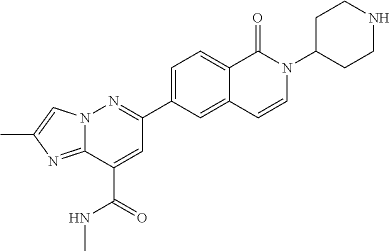 [SMILES: CNC(=O)c1cc(-c2ccc3c(=O)n(C4CCNCC4)ccc3c2)nn2cc(C)nc12] |
| 519 | 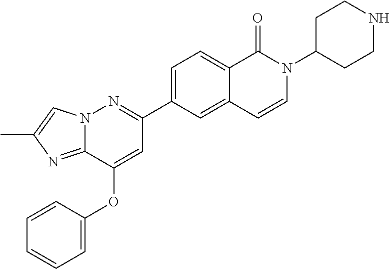 [SMILES: Cc1cn2nc(-c3ccc4c(=O)n(C5CCNCC5)ccc4c3)cc(Oc3ccccc3)c2n1] |
| 520 | 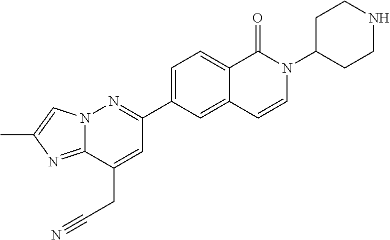 [SMILES: Cc1cn2nc(-c3ccc4c(=O)n(C5CCNCC5)ccc4c3)cc(CC#N)c2n1] |
| 521 | 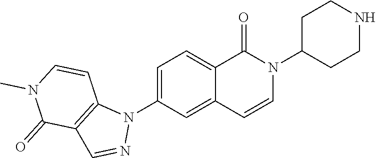 [SMILES: Cn1ccc2c(cnn2-c2ccc3c(=O)n(C4CCNCC4)ccc3c2)c1=O] |
| 522 | 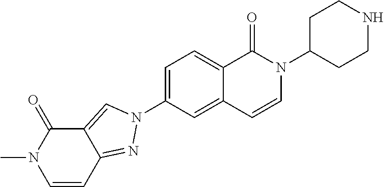 [SMILES: Cn1ccc2nn(-c3ccc4c(=O)n(C5CCNCC5)ccc4c3)cc2c1=O] |
| 523 | 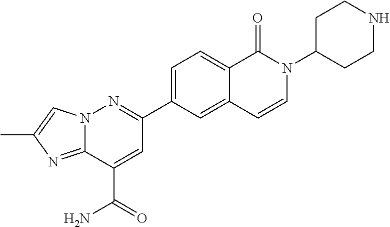 [SMILES: Cc1cn2nc(-c3ccc4c(=O)n(C5CCNCC5)ccc4c3)cc(C(N)=O)c2n1] |
| 524 | 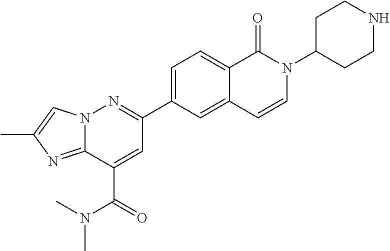 [SMILES: Cc1cn2nc(-c3ccc4c(=O)n(C5CCNCC5)ccc4c3)cc(C(=O)N(C)C)c2n1] |
| 525 | 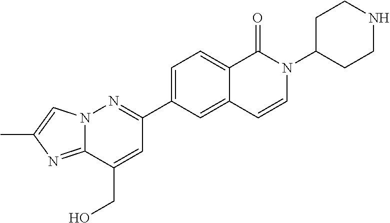 [SMILES: Cc1cn2nc(-c3ccc4c(=O)n(C5CCNCC5)ccc4c3)cc(CO)c2n1] |
| 526 | 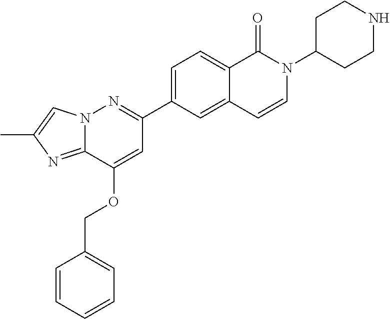 [SMILES: Cc1cn2nc(-c3ccc4c(=O)n(C5CCNCC5)ccc4c3)cc(OCc3ccccc3)c2n1] |
| 527 | 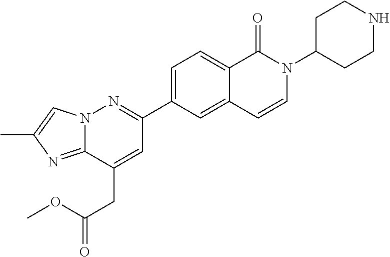 [SMILES: COC(=O)Cc1cc(-c2ccc3c(=O)n(C4CCNCC4)ccc3c2)nn2cc(C)nc12] |
| 528 | 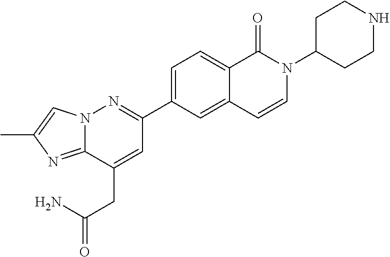 [SMILES: Cc1cn2nc(-c3ccc4c(=O)n(C5CCNCC5)ccc4c3)cc(CC(N)=O)c2n1] |
| 529 | 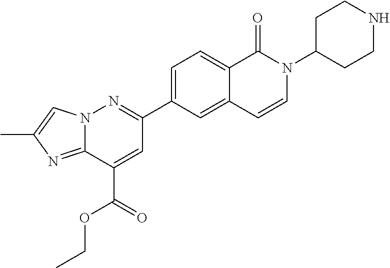 [SMILES: CCOC(=O)c1cc(-c2ccc3c(=O)n(C4CCNCC4)ccc3c2)nn2cc(C)nc12] |
| 530 | 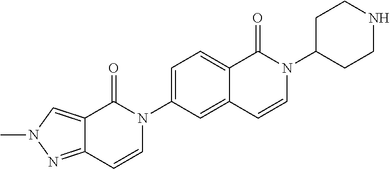 [SMILES: Cn1cc2c(=O)n(-c3ccc4c(=O)n(C5CCNCC5)ccc4c3)ccc2n1] |
| 531 | 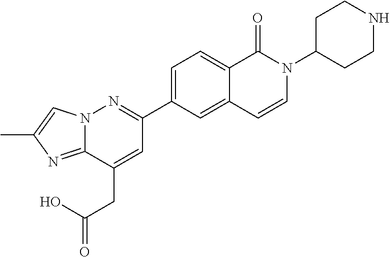 [SMILES: Cc1cn2nc(-c3ccc4c(=O)n(C5CCNCC5)ccc4c3)cc(CC(=O)O)c2n1] |
| 532 | 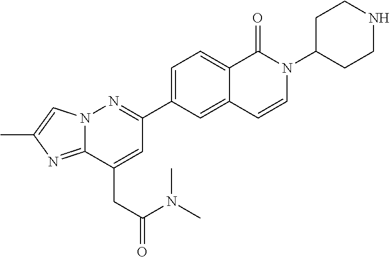 [SMILES: Cc1cn2nc(-c3ccc4c(=O)n(C5CCNCC5)ccc4c3)cc(CC(=O)N(C)C)c2n1] |
| 533 | 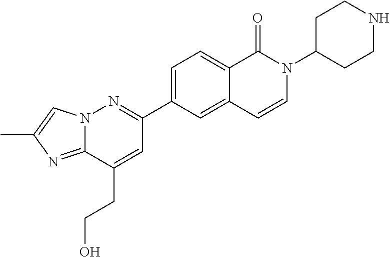 [SMILES: Cc1cn2nc(-c3ccc4c(=O)n(C5CCNCC5)ccc4c3)cc(CCO)c2n1] |
| 534 | 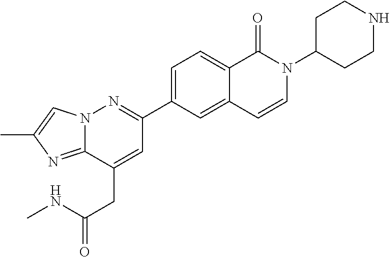 [SMILES: CNC(=O)Cc1cc(-c2ccc3c(=O)n(C4CCNCC4)ccc3c2)nn2cc(C)nc12] |
| 535 | 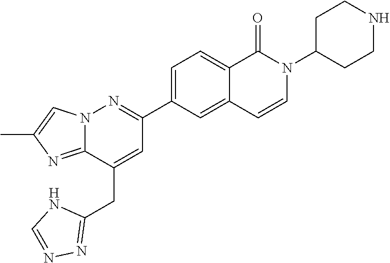 [SMILES: Cc1cn2nc(-c3ccc4c(=O)n(C5CCNCC5)ccc4c3)cc(Cc3nnc[nH]3)c2n1] |
| 536 | 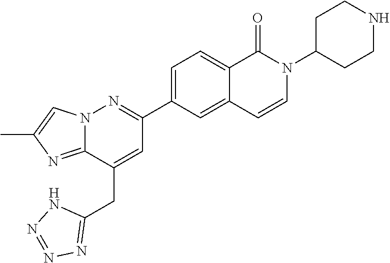 [SMILES: Cc1cn2nc(-c3ccc4c(=O)n(C5CCNCC5)ccc4c3)cc(Cc3nnn[nH]3)c2n1] |
| 537 | 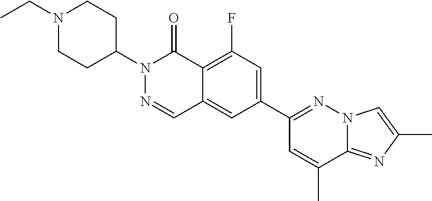 [SMILES: CCN1CCC(n2ncc3cc(-c4cc(C)c5nc(C)cn5n4)cc(F)c3c2=O)CC1] |
| 538 | 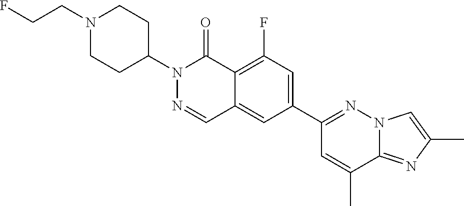 [SMILES: Cc1cn2nc(-c3cc(F)c4c(=O)n(C5CCN(CCF)CC5)ncc4c3)cc(C)c2n1] |
| 539 | 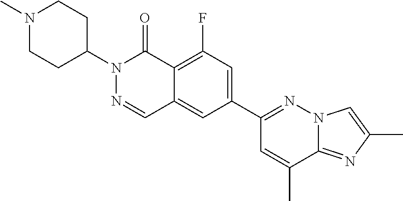 |
| 540 | 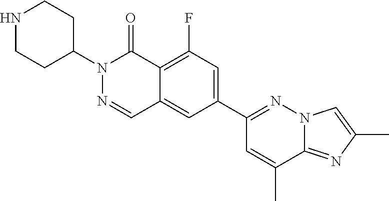 [SMILES: Cc1cn2nc(-c3cc(F)c4c(=O)n(C5CCNCC5)ncc4c3)cc(C)c2n1] |
| 541 | 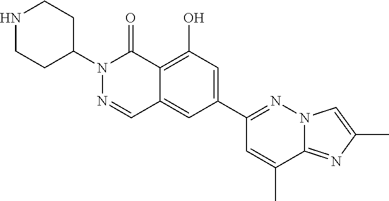 [SMILES: Cc1cn2nc(-c3cc(O)c4c(=O)n(C5CCNCC5)ncc4c3)cc(C)c2n1] |
| 542 | 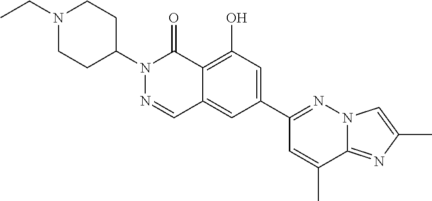 [SMILES: CCN1CCC(n2ncc3cc(-c4cc(C)c5nc(C)cn5n4)cc(O)c3c2=O)CC1] |
| 543 | 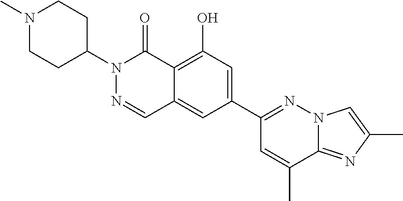 |
| 544 | 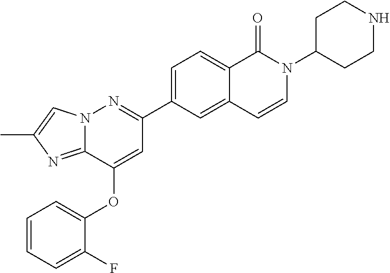 [SMILES: Cc1cn2nc(-c3ccc4c(=O)n(C5CCNCC5)ccc4c3)cc(Oc3ccccc3F)c2n1] |
| 545 | 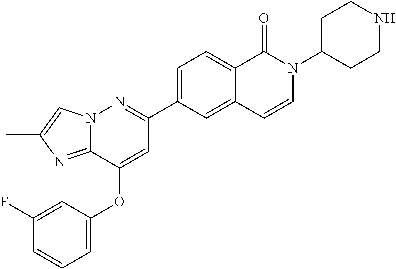 [SMILES: Cc1cn2nc(-c3ccc4c(=O)n(C5CCNCC5)ccc4c3)cc(Oc3cccc(F)c3)c2n1] |
| 546 | 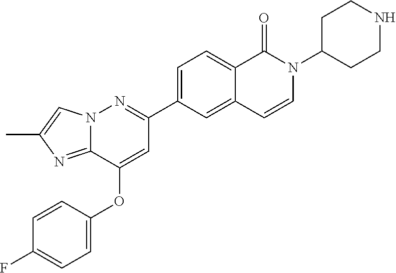 [SMILES: Cc1cn2nc(-c3ccc4c(=O)n(C5CCNCC5)ccc4c3)cc(Oc3ccc(F)cc3)c2n1] |
| 547 | 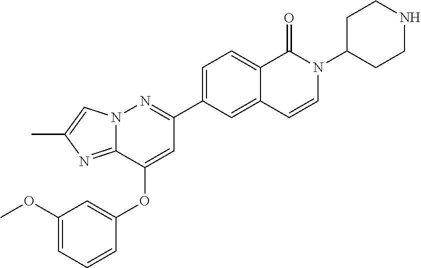 [SMILES: COc1cccc(Oc2cc(-c3ccc4c(=O)n(C5CCNCC5)ccc4c3)nn3cc(C)nc23)c1] |
| 548 | 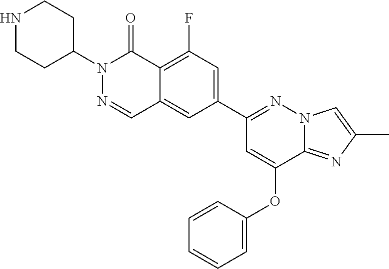 [SMILES: Cc1cn2nc(-c3cc(F)c4c(=O)n(C5CCNCC5)ncc4c3)cc(Oc3ccccc3)c2n1] |
| 549 | 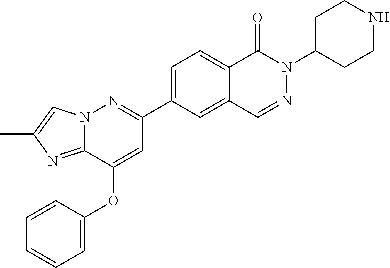 [SMILES: Cc1cn2nc(-c3ccc4c(=O)n(C5CCNCC5)ncc4c3)cc(Oc3ccccc3)c2n1] |
| 550 | 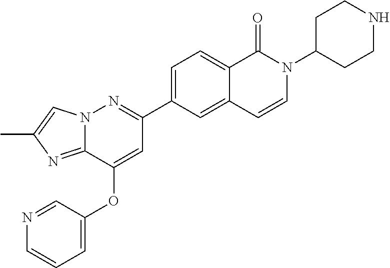 [SMILES: Cc1cn2nc(-c3ccc4c(=O)n(C5CCNCC5)ccc4c3)cc(Oc3cccnc3)c2n1] |
| 551 | 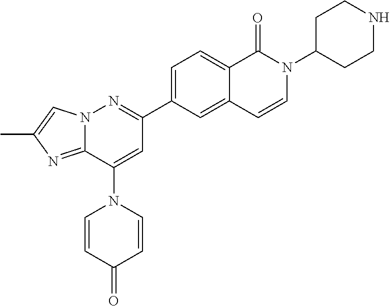 [SMILES: Cc1cn2nc(-c3ccc4c(=O)n(C5CCNCC5)ccc4c3)cc(-n3ccc(=O)cc3)c2n1] |
| 552 | 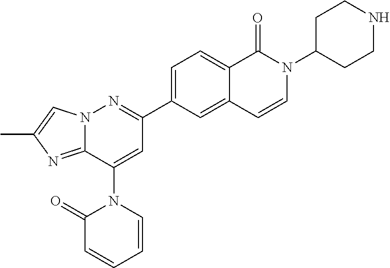 [SMILES: Cc1cn2nc(-c3ccc4c(=O)n(C5CCNCC5)ccc4c3)cc(-n3ccccc3=O)c2n1] |
| 553 | 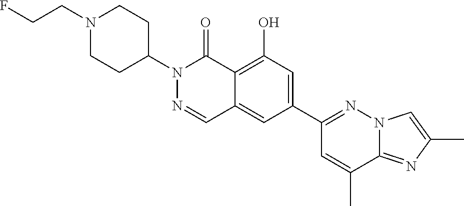 [SMILES: Cc1cn2nc(-c3cc(O)c4c(=O)n(C5CCN(CCF)CC5)ncc4c3)cc(C)c2n1] |
| 554 | 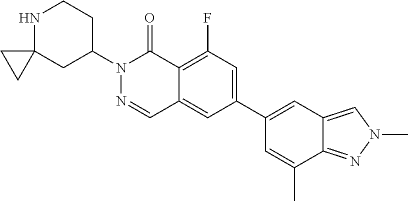 [SMILES: Cc1cc(-c2cc(F)c3c(=O)n(C4CCNC5(CC5)C4)ncc3c2)cc2cn(C)nc12] |
| 555 | 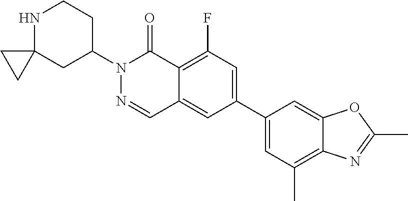 [SMILES: Cc1nc2c(C)cc(-c3cc(F)c4c(=O)n(C5CCNC6(CC6)C5)ncc4c3)cc2o1] |
| 556 | 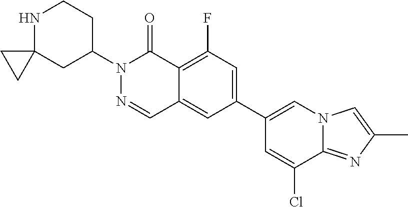 [SMILES: Cc1cn2cc(-c3cc(F)c4c(=O)n(C5CCNC6(CC6)C5)ncc4c3)cc(Cl)c2n1] |
| 557 | 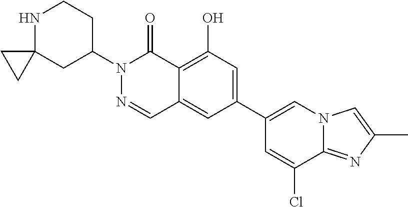 [SMILES: Cc1cn2cc(-c3cc(O)c4c(=O)n(C5CCNC6(CC6)C5)ncc4c3)cc(Cl)c2n1] |
| 558 | 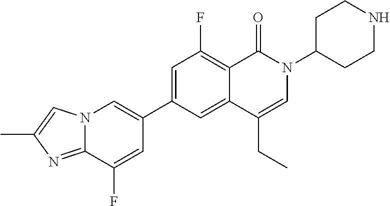 [SMILES: CCc1cn(C2CCNCC2)c(=O)c2c(F)cc(-c3cc(F)c4nc(C)cn4c3)cc12] |
| 559 | 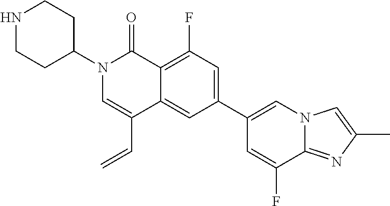 [SMILES: C=Cc1cn(C2CCNCC2)c(=O)c2c(F)cc(-c3cc(F)c4nc(C)cn4c3)cc12] |
| 560 | 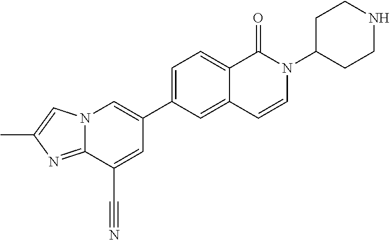 [SMILES: Cc1cn2cc(-c3ccc4c(=O)n(C5CCNCC5)ccc4c3)cc(C#N)c2n1] |
| 561 | 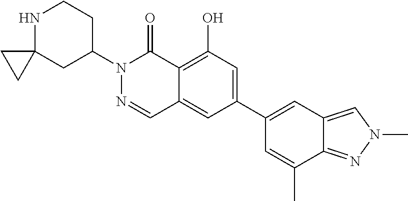 [SMILES: Cc1cc(-c2cc(O)c3c(=O)n(C4CCNC5(CC5)C4)ncc3c2)cc2cn(C)nc12] |
| 562 | 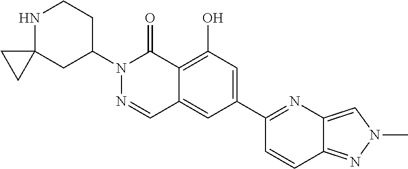 [SMILES: Cn1cc2nc(-c3cc(O)c4c(=O)n(C5CCNC6(CC6)C5)ncc4c3)ccc2n1] |
| 563 | 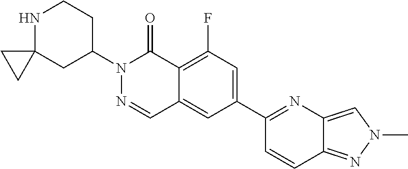 [SMILES: Cn1cc2nc(-c3cc(F)c4c(=O)n(C5CCNC6(CC6)C5)ncc4c3)ccc2n1] |
| 564 | 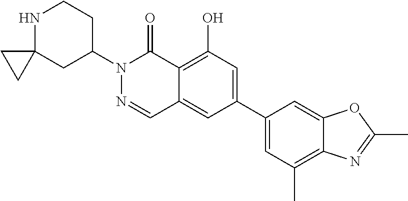 [SMILES: Cc1nc2c(C)cc(-c3cc(O)c4c(=O)n(C5CCNC6(CC6)C5)ncc4c3)cc2o1] |
| 565 | 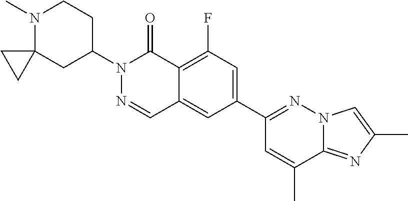 [SMILES: Cc1cn2nc(-c3cc(F)c4c(=O)n(C5CCN(C)C6(CC6)C5)ncc4c3)cc(C)c2n1] |
| 566 | 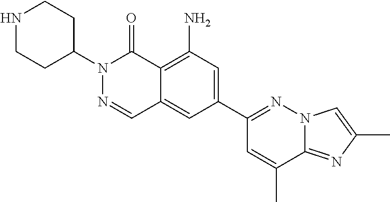 [SMILES: Cc1cn2nc(-c3cc(N)c4c(=O)n(C5CCNCC5)ncc4c3)cc(C)c2n1] |
| 567 | 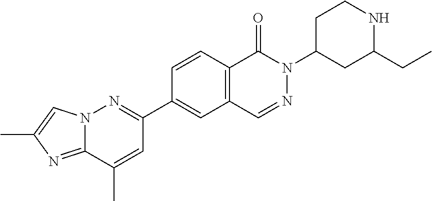 [SMILES: CCC1CC(n2ncc3cc(-c4cc(C)c5nc(C)cn5n4)ccc3c2=O)CCN1] |
| 568 | 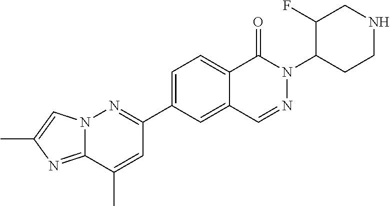 [SMILES: Cc1cn2nc(-c3ccc4c(=O)n(C5CCNCC5F)ncc4c3)cc(C)c2n1] |
| 569 | 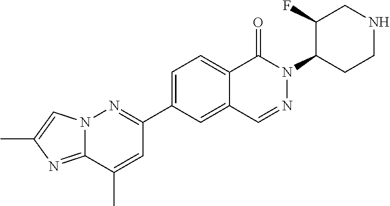 [SMILES: Cc1cn2nc(-c3ccc4c(=O)n([C@@H]5CCNC[C@@H]5F)ncc4c3)cc(C)c2n1] |
| 570 | 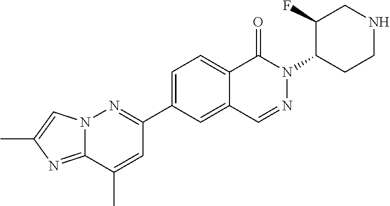 [SMILES: Cc1cn2nc(-c3ccc4c(=O)n([C@H]5CCNC[C@@H]5F)ncc4c3)cc(C)c2n1] |
| 571 | 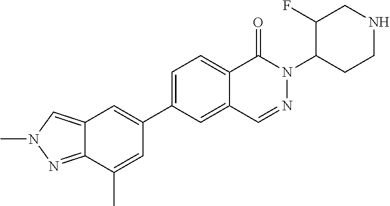 [SMILES: Cc1cc(-c2ccc3c(=O)n(C4CCNCC4F)ncc3c2)cc2cn(C)nc12] |
| 572 | 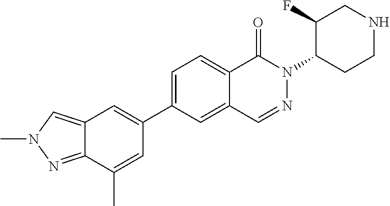 [SMILES: Cc1cc(-c2ccc3c(=O)n([C@H]4CCNC[C@@H]4F)ncc3c2)cc2cn(C)nc12] |
| 573 | 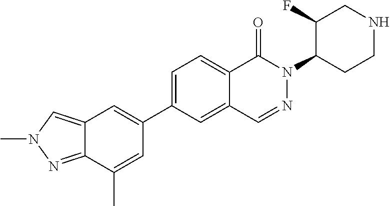 [SMILES: Cc1cc(-c2ccc3c(=O)n([C@@H]4CCNC[C@@H]4F)ncc3c2)cc2cn(C)nc12] |
| 574 | 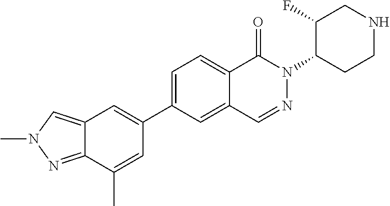 [SMILES: Cc1cc(-c2ccc3c(=O)n([C@H]4CCNC[C@H]4F)ncc3c2)cc2cn(C)nc12] |
| 575 | 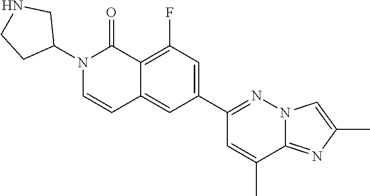 [SMILES: Cc1cn2nc(-c3cc(F)c4c(=O)n(C5CCNC5)ccc4c3)cc(C)c2n1] |
| 576 | 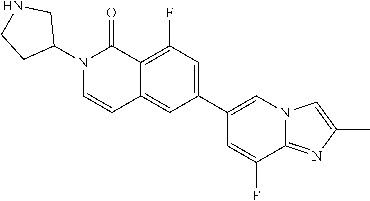 [SMILES: Cc1cn2cc(-c3cc(F)c4c(=O)n(C5CCNC5)ccc4c3)cc(F)c2n1] |
| 577 | 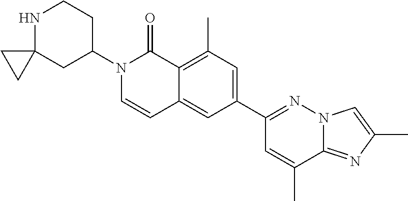 [SMILES: Cc1cn2nc(-c3cc(C)c4c(=O)n(C5CCNC6(CC6)C5)ccc4c3)cc(C)c2n1] |
| 578 | 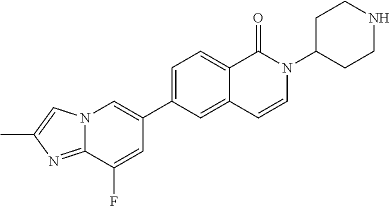 [SMILES: Cc1cn2cc(-c3ccc4c(=O)n(C5CCNCC5)ccc4c3)cc(F)c2n1] |
| 579 | 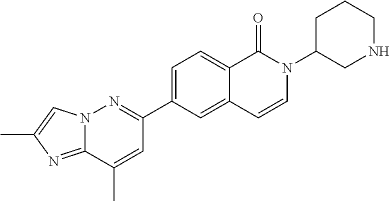 [SMILES: Cc1cn2nc(-c3ccc4c(=O)n(C5CCCNC5)ccc4c3)cc(C)c2n1] |
| 580 | 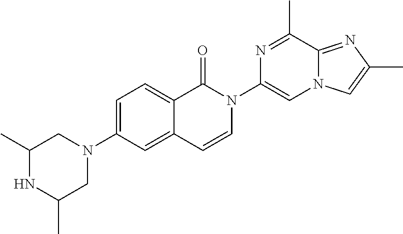 |
| 581 | 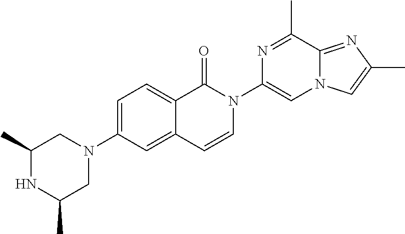 |
| 582 | 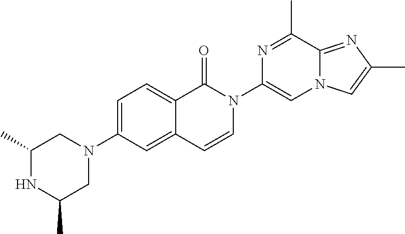 |
| 583 | 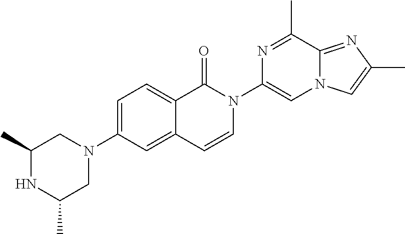 |
| 584 | 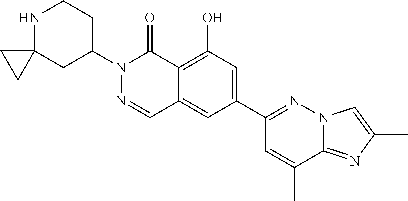 [SMILES: Cc1cn2nc(-c3cc(O)c4c(=O)n(C5CCNC6(CC6)C5)ncc4c3)cc(C)c2n1] |
| 610 | 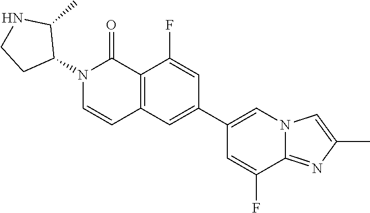 [SMILES: Cc1cn2cc(-c3cc(F)c4c(=O)n([C@@H]5CCN[C@@H]5C)ccc4c3)cc(F)c2n1] |
| 611 | 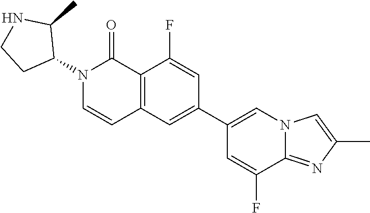 [SMILES: Cc1cn2cc(-c3cc(F)c4c(=O)n([C@@H]5CCN[C@H]5C)ccc4c3)cc(F)c2n1] |
| 612 | 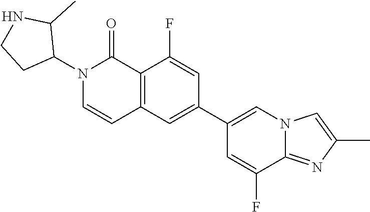 [SMILES: Cc1cn2cc(-c3cc(F)c4c(=O)n(C5CCNC5C)ccc4c3)cc(F)c2n1] |
| 613 | 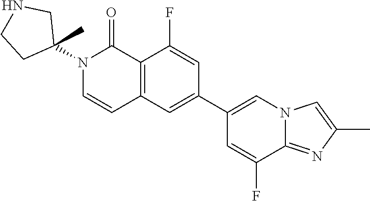 |
| 614 | 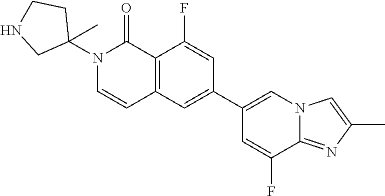 [SMILES: Cc1cn2cc(-c3cc(F)c4c(=O)n(C5(C)CCNC5)ccc4c3)cc(F)c2n1] |
| 615 | 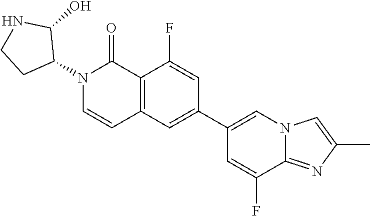 [SMILES: Cc1cn2cc(-c3cc(F)c4c(=O)n([C@@H]5CCN[C@@H]5O)ccc4c3)cc(F)c2n1] |
| 616 | 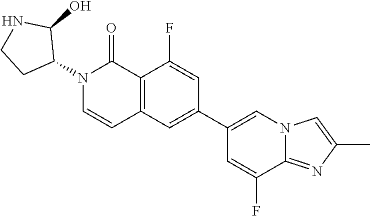 [SMILES: Cc1cn2cc(-c3cc(F)c4c(=O)n([C@@H]5CCN[C@H]5O)ccc4c3)cc(F)c2n1] |
| 617 | 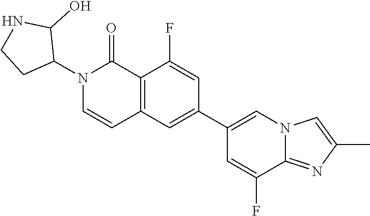 [SMILES: Cc1cn2cc(-c3cc(F)c4c(=O)n(C5CCNC5O)ccc4c3)cc(F)c2n1] |
| 618 | 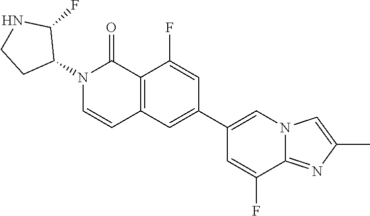 [SMILES: Cc1cn2cc(-c3cc(F)c4c(=O)n([C@@H]5CCN[C@@H]5F)ccc4c3)cc(F)c2n1] |
| 619 | 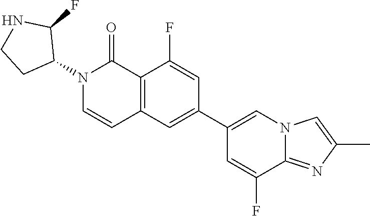 [SMILES: Cc1cn2cc(-c3cc(F)c4c(=O)n([C@@H]5CCN[C@H]5F)ccc4c3)cc(F)c2n1] |
| 620 | 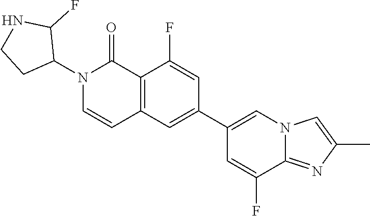 [SMILES: Cc1cn2cc(-c3cc(F)c4c(=O)n(C5CCNC5F)ccc4c3)cc(F)c2n1] |
| 621 | 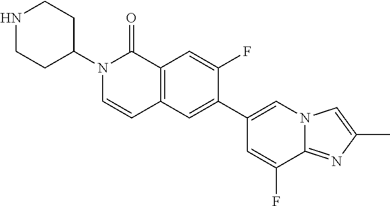 [SMILES: Cc1cn2cc(-c3cc4ccn(C5CCNCC5)c(=O)c4cc3F)cc(F)c2n1] |
| 622 | 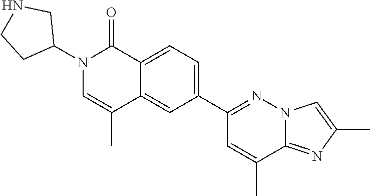 [SMILES: Cc1cn2nc(-c3ccc4c(=O)n(C5CCNC5)cc(C)c4c3)cc(C)c2n1] |
| 623 | 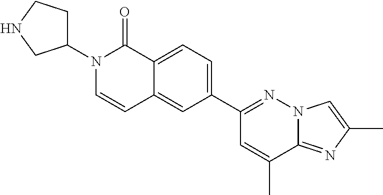 [SMILES: Cc1cn2nc(-c3ccc4c(=O)n(C5CCNC5)ccc4c3)cc(C)c2n1] |
| 624 | 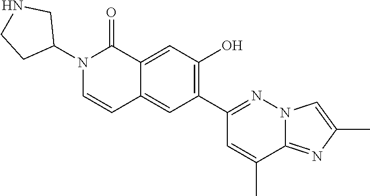 [SMILES: Cc1cn2nc(-c3cc4ccn(C5CCNC5)c(=O)c4cc3O)cc(C)c2n1] |
| 625 | 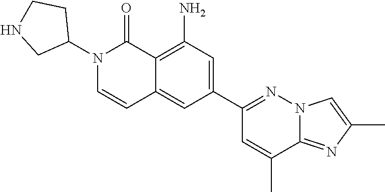 [SMILES: Cc1cn2nc(-c3cc(N)c4c(=O)n(C5CCNC5)ccc4c3)cc(C)c2n1] |
| 626 | 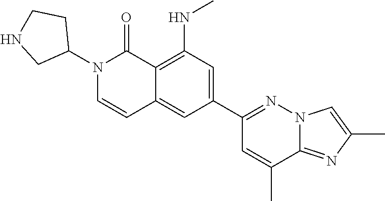 [SMILES: CNc1cc(-c2cc(C)c3nc(C)cn3n2)cc2ccn(C3CCNC3)c(=O)c12] |
| 627 | 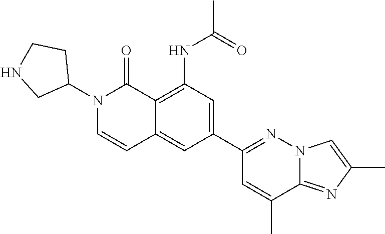 [SMILES: CC(=O)Nc1cc(-c2cc(C)c3nc(C)cn3n2)cc2ccn(C3CCNC3)c(=O)c12] |
| 628 | 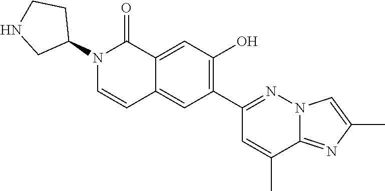 [SMILES: Cc1cn2nc(-c3cc4ccn([C@@H]5CCNC5)c(=O)c4cc3O)cc(C)c2n1] |
| 629 | 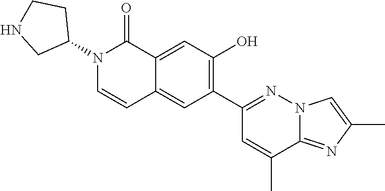 [SMILES: Cc1cn2nc(-c3cc4ccn([C@H]5CCNC5)c(=O)c4cc3O)cc(C)c2n1] |
| 630 | 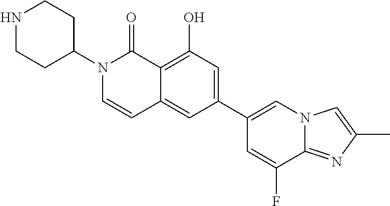 [SMILES: Cc1cn2cc(-c3cc(O)c4c(=O)n(C5CCNCC5)ccc4c3)cc(F)c2n1] |
| 631 | 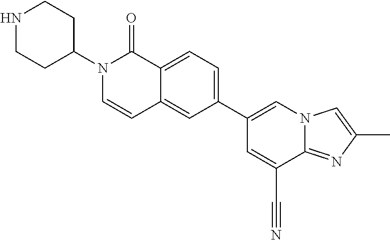 [SMILES: Cc1cn2cc(-c3ccc4c(=O)n(C5CCNCC5)ccc4c3)cc(C#N)c2n1] |
| 632 | 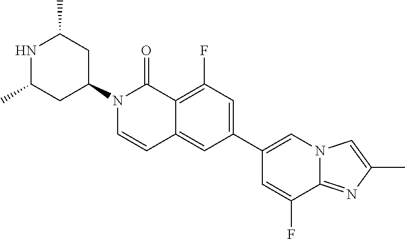 |
| 633 | 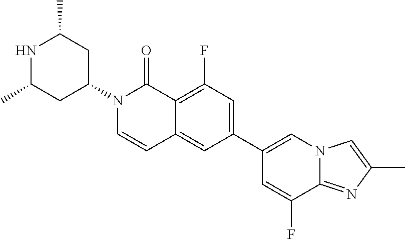 |
| 634 | 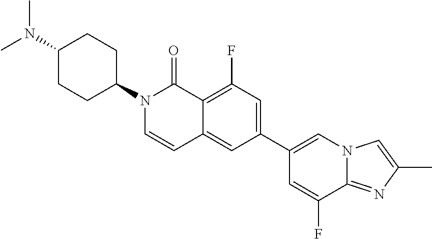 |
| 635 | 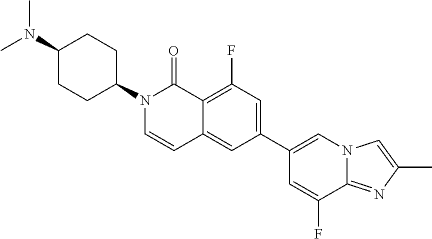 |
| 636 | 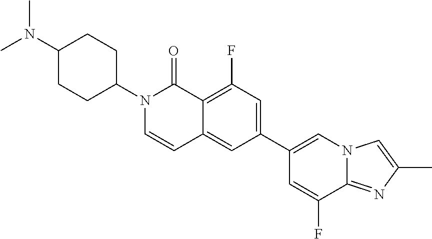 |
| 637 | 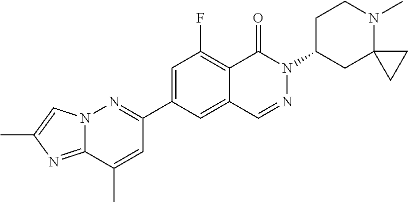 [SMILES: Cc1cn2nc(-c3cc(F)c4c(=O)n([C@@H]5CCN(C)C6(CC6)C5)ncc4c3)cc(C)c2n1] |
| 638 | 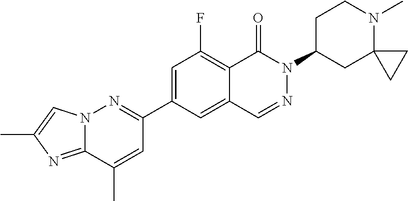 [SMILES: Cc1cn2nc(-c3cc(F)c4c(=O)n([C@H]5CCN(C)C6(CC6)C5)ncc4c3)cc(C)c2n1] |
| 639 | 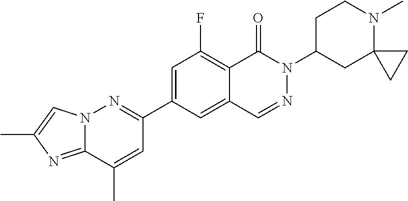 [SMILES: Cc1cn2nc(-c3cc(F)c4c(=O)n(C5CCN(C)C6(CC6)C5)ncc4c3)cc(C)c2n1] |
| 640 | 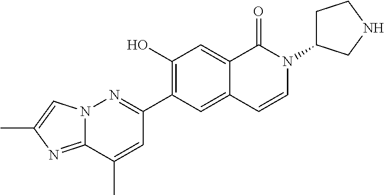 [SMILES: Cc1cn2nc(-c3cc4ccn([C@@H]5CCNC5)c(=O)c4cc3O)cc(C)c2n1] |
| 641 | 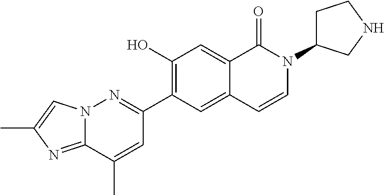 [SMILES: Cc1cn2nc(-c3cc4ccn([C@H]5CCNC5)c(=O)c4cc3O)cc(C)c2n1] |
| 642 | 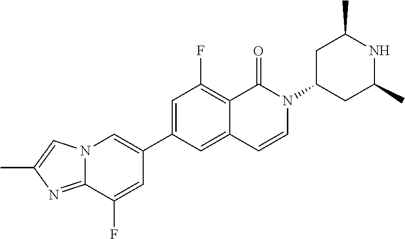 |
| 643 | 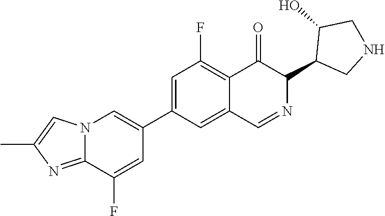 [SMILES: Cc1cn2cc(-c3cc(F)c4c(c3)C=NC([C@@H]3CNC[C@H]3O)C4=O)cc(F)c2n1] |
| 644 | 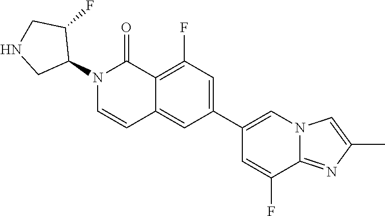 [SMILES: Cc1cn2cc(-c3cc(F)c4c(=O)n([C@H]5CNC[C@@H]5F)ccc4c3)cc(F)c2n1] |
| 645 | 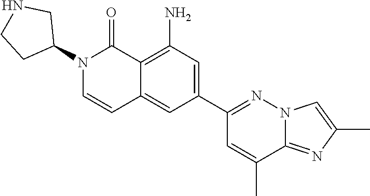 [SMILES: Cc1cn2nc(-c3cc(N)c4c(=O)n([C@H]5CCNC5)ccc4c3)cc(C)c2n1] |
| 646 | 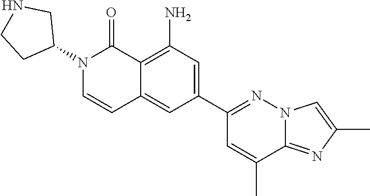 [SMILES: Cc1cn2nc(-c3cc(N)c4c(=O)n([C@@H]5CCNC5)ccc4c3)cc(C)c2n1] |
| 647 | 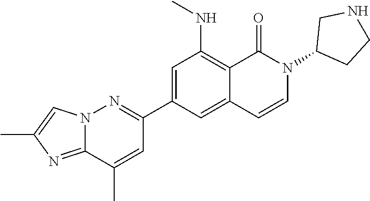 [SMILES: CNc1cc(-c2cc(C)c3nc(C)cn3n2)cc2ccn([C@H]3CCNC3)c(=O)c12] |
| 648 | 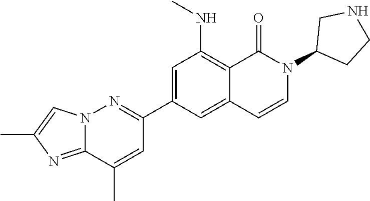 [SMILES: CNc1cc(-c2cc(C)c3nc(C)cn3n2)cc2ccn([C@@H]3CCNC3)c(=O)c12] |
| 649 | 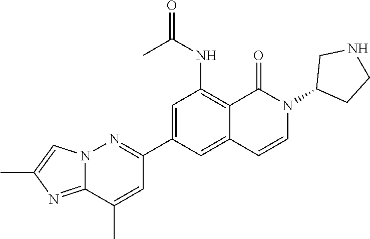 [SMILES: CC(=O)Nc1cc(-c2cc(C)c3nc(C)cn3n2)cc2ccn([C@H]3CCNC3)c(=O)c12] |
| 650 | 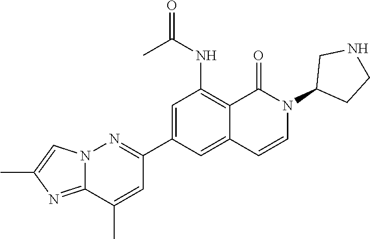 [SMILES: CC(=O)Nc1cc(-c2cc(C)c3nc(C)cn3n2)cc2ccn([C@@H]3CCNC3)c(=O)c12] |
| 651 | 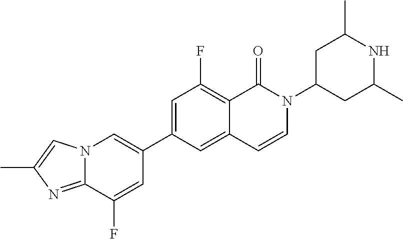 |
| 652 | 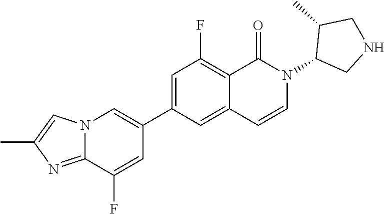 [SMILES: Cc1cn2cc(-c3cc(F)c4c(=O)n([C@H]5CNC[C@H]5C)ccc4c3)cc(F)c2n1] |
| 653 | 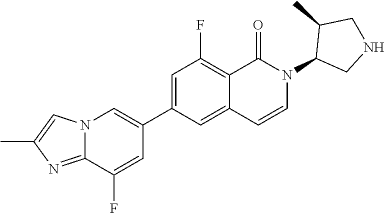 [SMILES: Cc1cn2cc(-c3cc(F)c4c(=O)n([C@@H]5CNC[C@@H]5C)ccc4c3)cc(F)c2n1] |
| 654 | 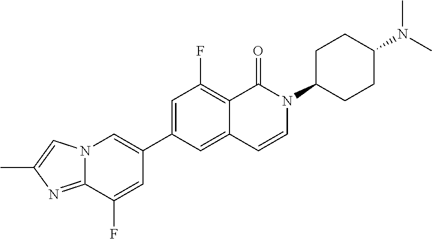 |
| 655 | 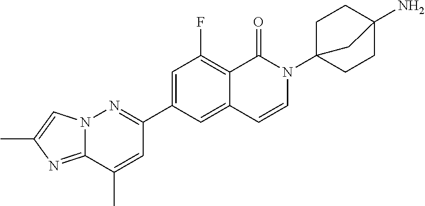 [SMILES: Cc1cn2nc(-c3cc(F)c4c(=O)n(C56CCC(N)(CC5)C6)ccc4c3)cc(C)c2n1] |
| 656 | 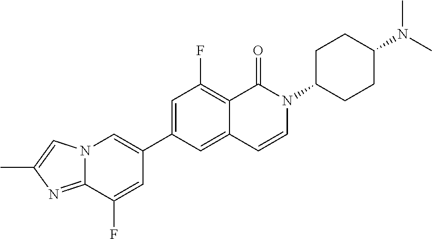 |

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 185, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is N; R² is hydrogen; m is 0; and n is 1. In some embodiments, the compound of Formula (I) is Compound 186, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is C(R^{6a})(R^{6b}) (e.g., CH₂); each R² is hydrogen; R³ is halo (e.g., F); m is 1; and n is 2. In some embodiments, the compound of Formula (I) is Compound 187, 188, or a pharmaceutically acceptable salt, solvate, hydrate, tautomer, or stereoisomer thereof.

In some embodiments, for Formula (I), A is bicyclic heteroaryl (e.g., 4-fluoro-2-methylbenzo[d]oxazolyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 215, or a pharmaceutically acceptable salt, solvate, hydrate, tautomer, or stereoisomer thereof.

In some embodiments, for Formula (I), A is bicyclic heteroaryl (e.g., 8-chloro-2-methylimidazo[1,2-a]pyridinyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 216, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-a]pyridinyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 217, or a pharmaceutically acceptable salt, solvate, hydrate, tautomer, or stereoisomer thereof.

In some embodiments, for Formula (I), A is bicyclic heteroaryl (e.g., 2,7-dimethylimidazo[1,2-a]pyridinyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 218, or a pharmaceutically acceptable salt, solvate, hydrate, tautomer, or stereoisomer thereof.

In some embodiments, for Formula (I), A is bicyclic heteroaryl (e.g., 2,7-dimethyl-2H-indazolyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 219, or a pharmaceutically acceptable salt, solvate, hydrate, tautomer, or stereoisomer thereof.

In some embodiments, for Formula (I), A is monocyclic heteroaryl (e.g., pyrazolyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 220, or a pharmaceutically acceptable salt, solvate, hydrate, tautomer, or stereoisomer thereof.

In some embodiments, for Formula (I), A is bicyclic heteroaryl (e.g., 6,8-dimethylimidazo[1,2-a]pyrazyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 221, or a pharmaceutically acceptable salt, solvate, hydrate, tautomer, or stereoisomer thereof.

In some embodiments, for Formula (I), A is bicyclic heteroaryl (e.g., 6,8-dimethyl-[1,2,4]triazolo[1,5-a]pyrazyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 222, or a pharmaceutically acceptable salt, solvate, hydrate, tautomer, or stereoisomer thereof.

In some embodiments, for Formula (I), A is bicyclic heteroaryl (e.g., 4,6-dimethylpyrazolo[1,5-a]pyrazyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 223, or a pharmaceutically acceptable salt, solvate, hydrate, tautomer, or stereoisomer thereof.

In some embodiments, for Formula (I), A is bicyclic heteroaryl (e.g., 2-methylimidazo[1,2-a]pyrazyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 224, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 225, or a pharmaceutically acceptable salt, solvate, hydrate, tautomer, or stereoisomer thereof.

In some embodiments, for Formula (I), A is bicyclic heteroaryl (e.g., 4-fluoro-2-methylbenzo[d]thiazolyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 226, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 247, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (I) is Compound 248, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., piperidinyl); L¹ and L² are absent; Y is N; R² is hydrogen; m is 0; and n is 1. In some embodiments, the compound of Formula (I) is Compound 249, or a pharmaceutically acceptable salt, solvate, hydrate, tautomer, or stereoisomer thereof.

In some embodiments, for Formula (I), A is bicyclic heteroaryl (e.g., 4-fluoro-2-methylbenzo[d]oxazolyl); B is monocyclic heterocyclyl (e.g., piperidinyl); L¹ and L² are absent; Y is N; R² is hydrogen; m is 0; and n is 1. In some embodiments, the compound of Formula (I) is Compound 250, or a pharmaceutically acceptable salt, solvate, hydrate, tautomer, or stereoisomer thereof.

In some embodiments, for Formula (I), A is bicyclic heteroaryl (e.g., 2,7-dimethyl-2H-indazolyl); B is monocyclic heterocyclyl (e.g., piperidinyl); L¹ and L² are absent; Y is N; R² is hydrogen; m is 0; and n is 1. In some embodiments, the compound of Formula (I) is Compound 251, or a pharmaceutically acceptable salt, solvate, hydrate, tautomer, or stereoisomer thereof.

In some embodiments, for Formula (I), A is bicyclic heteroaryl (e.g., 2-methyl-8-(trifluoromethyl)imidazo[1,2-a]pyridinyl); B is monocyclic heterocyclyl (e.g., piperidinyl); L¹ and L² are absent; Y is C(R^{6a})(R^{6b}) (e.g., CH₂); R² is hydrogen; m is 0; and n is 1. In some embodiments, the compound of Formula (I) is Compound 252, or a pharmaceutically acceptable salt, solvate, hydrate, tautomer, or stereoisomer thereof.

In some embodiments, for Formula (I), A is bicyclic heteroaryl (e.g., 4-fluoro-2-methylbenzo[d]thiazolyl); B is monocyclic heterocyclyl (e.g., piperidinyl); L¹ and L² are absent; Y is N; R² is hydrogen; m is 0; and n is 1. In some embodiments, the compound of Formula (I) is Compound 253, or a pharmaceutically acceptable salt, solvate, hydrate, tautomer, or stereoisomer thereof.

In some embodiments, for Formula (I), A is bicyclic heteroaryl (e.g., 2,7-dimethylimidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (I) is Compound 254, or a pharmaceutically acceptable salt, solvate, hydrate, tautomer, or stereoisomer thereof.

In some embodiments, for Formula (I), A is bicyclic heteroaryl (e.g., 2-methylimidazo[1,2-a]pyrazyl); B is monocyclic heterocyclyl (e.g., piperidinyl); L¹ and L² are absent; Y is N; R² is hydrogen; m is 0; and n is 1. In some embodiments, the compound of Formula (I) is Compound 255, or a pharmaceutically acceptable salt, solvate, hydrate, tautomer, or stereoisomer thereof.

In some embodiments, for Formula (I), A is bicyclic heteroaryl (e.g., 8-chloro-2-methylimidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (I) is Compound 256, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (I) is Compound 257, or a pharmaceutically acceptable salt, solvate, hydrate, tautomer, or stereoisomer thereof.

In some embodiments, for Formula (I), A is bicyclic heteroaryl (e.g., 6,8-dimethylimidazo[1,2-a]pyrazyl); B is monocyclic heterocyclyl (e.g., piperidinyl); L¹ and L² are absent; Y is N; R² is hydrogen; m is 0; and n is 1. In some embodiments, the compound of Formula (I) is Compound 258, or a pharmaceutically acceptable salt, solvate, hydrate, tautomer, or stereoisomer thereof.

In some embodiments, for Formula (I), A is bicyclic heteroaryl (e.g., 6,8-dimethyl-[1,2,4]triazolo[1,5-a]pyrazyl); B is monocyclic heterocyclyl (e.g., piperidinyl); L¹ and L² are absent; Y is N; R² is hydrogen; m is 0; and n is 1. In some embodiments, the compound of Formula (I) is Compound 259, or a pharmaceutically acceptable salt, solvate, hydrate, tautomer, or stereoisomer thereof.

In some embodiments, for Formula (I), A is bicyclic heteroaryl (e.g., 4,6-dimethylpyrazolo[1,5-a]pyrazyl); B is monocyclic heterocyclyl (e.g., piperidinyl); L¹ and L² are absent; Y is N; R² is hydrogen; m is 0; and n is 1. In some embodiments, the compound of Formula (I) is Compound 260, or a pharmaceutically acceptable salt, solvate, hydrate, tautomer, or stereoisomer thereof.

In some embodiments, for Formula (I), A is monocyclic heteroaryl (e.g., pyrazyl); B is monocyclic heterocyclyl (e.g., piperidinyl); L¹ and L² are absent; Y is N; R² is hydrogen; m is 0; and n is 1. In some embodiments, the compound of Formula (I) is Compound 261, or a pharmaceutically acceptable salt, solvate, hydrate, tautomer, or stereoisomer thereof.

In some embodiments, for Formula (I), A is bicyclic heteroaryl (e.g., 2-methyl-8-(trifluoromethyl)imidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (I) is Compound 262, or a pharmaceutically acceptable salt, solvate, hydrate, tautomer, or stereoisomer thereof.

In some embodiments, for Formula (I), A is bicyclic heteroaryl (e.g., 2-methyl-2H-indazolyl); B is monocyclic heterocyclyl (e.g., piperidinyl); L¹ and L² are absent; Y is N; R² is hydrogen; m is 0; and n is 1. In some embodiments, the compound of Formula (I) is Compound 263, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., pyrrolidinyl); L¹ and L² are absent; Y is N; R² is hydrogen; m is 0; and n is 1. In some embodiments, the compound of Formula (I) is Compound 264, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-methyl piperidinyl); L¹ and L² are absent; Y is N; R² is hydrogen; m is 0; and n is 1. In some embodiments, the compound of Formula (I) is Compound 265, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-ethyl piperidinyl); L¹ and L² are absent; Y is N; R² is hydrogen; m is 0; and n is 1. In some embodiments, the compound of Formula (I) is Compound 266, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 2,2-dimethylpiperidinyl); L¹ and L² are absent; Y is C(R^{6a})(R^{6b}) (e.g., CH₂); each R² is hydrogen; m is 0; and n is 2. In some embodiments, the compound of Formula (I) is Compound 267, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-methyl piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 268, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., pyrrolidinyl); L¹ and L² are absent; Y is C(R^{6a})(R^{6b}) (e.g., CH₂); each R² is hydrogen; m is 0; and n is 2. In some embodiments, the compound of Formula (I) is Compound 269, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-ethyl piperidinyl); L¹ and L² are absent; Y is C(R^{6a})(R^{6b}) (e.g., CH₂); each R² is hydrogen; m is 0; and n is 2. In some embodiments, the compound of Formula (I) is Compound 270, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-methyl pyrrolidinyl); L¹ and L² are absent; Y is N; R² is hydrogen; m is 0; and n is 1. In some embodiments, the compound of Formula (I) is Compound 271, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 2-methylpiperidine); L¹ and L² are absent; Y is N; R² is hydrogen; m is 0; and n is 1. In some embodiments, the compound of Formula (I) is Compound 272, 273, 324, 328, 329, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 4-azaspiro[2.5]octanyl); L¹ and L² are absent; Y is N; R² is hydrogen; m is 0; and n is 1. In some embodiments, the compound of Formula (I) is Compound 274, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., tetrahydro-2H-pyranyl); L¹ and L² are absent; Y is N; R² is hydrogen; m is 0; and n is 1. In some embodiments, the compound of Formula (I) is Compound 275, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L¹ and L² are absent; Y is C(R^{6a}) (e.g., CH); R² is hydrogen; m is 0; and n is 1. In some embodiments, the compound of Formula (I) is Compound 276, 403, 404, 578, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-methyl 2-methylpiperidine); L¹ and L² are absent; Y is N; R² is hydrogen; m is 0; and n is 1. In some embodiments, the compound of Formula (I) is Compound 277, 278, 325, 330, 331, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 2,2-dimethylpiperidinyl); L¹ and L² are absent; Y is N; R² is hydrogen; m is 0; and n is 1. In some embodiments, the compound of Formula (I) is Compound 279, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 8-azabicyclo[3.2.1]octanyl); L¹ and L² are absent; Y is N; R² is hydrogen; m is 0; and n is 1. In some embodiments, the compound of Formula (I) is Compound 280, 326, 332, 333, 334 or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 2-methylpiperidinyl); L¹ and L² are absent; Y is C(R^{6a})(R^{6b}) (e.g., CH₂); each R² is hydrogen; m is 0; and n is 2. In some embodiments, the compound of Formula (I) is Compound 281, 327, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-methyl pyrrolidinyl); L¹ and L² are absent; Y is C(R^{6a})(R^{6b}) (e.g., CH₂); each R² is hydrogen; m is 0; and n is 2. In some embodiments, the compound of Formula (I) is Compound 282, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., azepanyl); L¹ and L² are absent; Y is N; R² is hydrogen; m is 0; and n is 1. In some embodiments, the compound of Formula (I) is Compound 283, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 2-ethylpiperidinyl); L¹ and L² are absent; Y is N; R² is hydrogen; m is 0; and n is 1. In some embodiments, the compound of Formula (I) is Compound 304, 305, 328, 335, 336, 567, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is C(R^{6a}) (e.g., CH); R² is hydrogen; m is 0; and n is 1. In some embodiments, the compound of Formula (I) is Compound 309, 410, 411, 579, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-methyl piperidinyl); L¹ and L² are absent; Y is C(R^{6a}) (e.g., CH); R² is hydrogen; m is 0; and n is 1. In some embodiments, the compound of Formula (I) is Compound 310, or a pharmaceutically acceptable salt, solvate, hydrate, tautomer, or stereoisomer thereof.

In some embodiments, for Formula (I), A is bicyclic heteraryl (e.g., 2,8-dimethylimidazo[1,2-b]pyridazyl); B is monocyclic heterocyclyl (e.g., 4-azaspiro[2.5]octanyl); L¹ and L² are absent; Y is N; R² is hydrogen; R³ is halo (e.g., F); m is 1; and n is 1. In some embodiments, the compound of Formula (I) is Compound 312, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is C(R^{6a}) (e.g., CH); R² is hydrogen; R³ is halo (e.g., F); m is 1; and n is 1. In some embodiments, the compound of Formula (I) is Compound 322, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 2-methylpiperidinyl); L¹ and L² are absent; Y is N; R² and R³ are independently hydrogen; m is 1; and n is 1. In some embodiments, the compound of Formula (I) is Compound 324, Compound 328, Compound 329, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 1,2-dimethylpiperidinyl); L¹ and L² are absent; Y is N; R² and R³ are independently hydrogen; m is 1; and n is 1. In some embodiments, the compound of Formula (I) is Compound 325, Compound 330, Compound 331 or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 8-azabicyclo[3.2.1]octanyl); L¹ and L² are absent; Y is N; R² and R³ are independently hydrogen; m is 1; and n is 1. In some embodiments, the compound of Formula (I) is Compound 326, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 2-methylpiperidinyl); L¹ and L² are absent; Y is C(R^{6a}) (e.g., CH₂); R² and R³ are independently hydrogen; m is 1; and n is 1. In some embodiments, the compound of Formula (I) is Compound 327, Compound 332, Compound 333, Compound 334, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 2-ethylpiperidinyl); L¹ and L² are absent; Y is N; R² and R³ are independently hydrogen; m is 1; and n is 1. In some embodiments, the compound of Formula (I) is Compound 335, Compound 336, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; Y is N; R² is hydrogen; R³ is hydrogen; m is 1; and n is 1. In some embodiments, the compound of Formula (I) is Compound 368, Compound 369, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L¹ and L² are absent; Y is C(R^{6a}) (e.g., CH); R² is hydrogen; R³ is halo (e.g., F); m is 1; and n is 1. In some embodiments, the compound of Formula (I) is Compound 370, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 3-fluoropiperidinyl); L¹ and L² are absent; Y is N; R² is hydrogen; R³ is hydrogen; m is 1; and n is 1. In some embodiments, the compound of Formula (I) is Compound 372, 568, 569, 570, or a pharmaceutically acceptable salt, solvate, hydrate, tautomer, or stereoisomer thereof.

In some embodiments, for Formula (I), A is bicyclic heteroaryl (e.g., 2,7-dimethyl-2H-imidazolyl); B is bicyclic heterocyclyl (e.g., 2-oxa-5-azaspiro[3.5]nonanyl); L¹ and L² are absent; Y is N; R² is hydrogen; R³ is hydrogen; m is 1; and n is 1. In some embodiments, the compound of Formula (I) is Compound 375, or a pharmaceutically acceptable salt, solvate, hydrate, tautomer, or stereoisomer thereof.

In some embodiments, for Formula (I), A is bicyclic heteroaryl (e.g., 2,7-dimethyl-2H-imidazolyl); B is monocyclic heterocyclyl (e.g., 3-fluoropiperidinyl); L¹ and L² are absent; Y is N; R² is hydrogen; R³ is hydrogen; m is 1; and n is 1. In some embodiments, the compound of Formula (I) is Compound 378, 571, 572, 573, 574, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-b]pyridazinyl); B is monocyclic heterocyclyl (e.g., 3-fluoropiperidinyl); L¹ and L² are absent; Y is N; R² is hydrogen; R³ is hydrogen; m is 1; and n is 1. In some embodiments, the compound of Formula (I) is Compound 379, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 380, Compound 425, Compound 426, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is C(R^{6a}) (e.g., CH); R² is methyl; R³ is hydrogen; m is 1; and n is 1. In some embodiments, the compound of Formula (I) is Compound 381 or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazinyl); B is monocyclic heterocyclyl (e.g., pyrrolidinyl); L¹ and L² are absent; Y is C(R^{6a}) (e.g., CH); R² is hydrogen; R³ is halo (e.g., F); m is 1; and n is 1. In some embodiments, the compound of Formula (I) is Compound 382, Compound 392, 575 or a pharmaceutically acceptable salt, solvate, hydrate, tautomer, or stereoisomer thereof.

In some embodiments, for Formula (I), A is bicyclic heteroaryl (e.g., 8-cyano-2-methylimidazo[1,2-a]pyridinyl); B is monocyclic heterocyclyl (e.g., piperidinyl); L¹ and L² are absent; Y is C(R^{6a}) (e.g., CH); R² is hydrogen; R³ is halo (e.g., F); m is 1; and n is 1. In some embodiments, the compound of Formula (I) is Compound 383 or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; Y is C(R^{6a}) (e.g., CH); R² and R³ are each hydrogen; m is 1; and n is 1. In some embodiments, the compound of Formula (I) is Compound 384 or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; Y is C(R^{6a}) (e.g., CH); R² is hydrogen; R³ is cyano; m is 1; and n is 1. In some embodiments, the compound of Formula (I) is Compound 387 or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is C(R^{6a}) (e.g., CH); R² is hydrogen; R³ is halo (e.g., C₁); m is 1; and n is 1. In some embodiments, the compound of Formula (I) is Compound 390 or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is C(R^{6a}) (e.g., CH); R² is hydrogen; R³ is hydroxy; m is 1; and n is 1. In some embodiments, the compound of Formula (I) is Compound 391, Compound 448, or a pharmaceutically acceptable salt, solvate, hydrate, tautomer, or stereoisomer thereof.

In some embodiments, for Formula (I), A is bicyclic heteroaryl (e.g., 1-(2-methylimidazo[1,2-a]pyridin-8-yl)ethanoyl); B is monocyclic heterocyclyl (e.g., piperidinyl); L¹ and L² are absent; Y is C(R^{6a}) (e.g., CH); R² is hydrogen; R³ is halo (e.g., F); m is 1; and n is 1. In some embodiments, the compound of Formula (I) is Compound 393 or a pharmaceutically acceptable salt, solvate, hydrate, tautomer, or stereoisomer thereof.

In some embodiments, for Formula (I), A is bicyclic heteroaryl (e.g., 7-cyano-2-methyl-2H-indazolyl); B is monocyclic heterocyclyl (e.g., piperidinyl); L¹ and L² are absent; Y is C(R^{6a}) (e.g., CH); R² is hydrogen; R³ is halo (e.g., F); m is 1; and n is 1. In some embodiments, the compound of Formula (I) is Compound 394 or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., pyrrolidinyl); L¹ and L² are absent; Y is C(R^{6a}) (e.g., CH); R² is hydrogen; R³ is halo (e.g., F); m is 1; and n is 1. In some embodiments, the compound of Formula (I) is Compound 395, Compound 396, 576 or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; Y is C(R^{6a}) (e.g., CH); R² is hydrogen; R³ is methyl; m is 1; and n is 1. In some embodiments, the compound of Formula (I) is Compound 397, Compound 398, 577, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is C(R^{6a}) (e.g., CH); R² is hydrogen; R³ is methoxy; m is 1; and n is 1. In some embodiments, the compound of Formula (I) is Compound 399, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-methyl piperidinyl); L¹ and L² are absent; Y is C(R^{6a}) (e.g., CH); R² is hydrogen; R³ is halo (e.g., F); m is 1; and n is 1. In some embodiments, the compound of Formula (I) is Compound 400, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-ethyl piperidinyl); L¹ and L² are absent; Y is C(R^{6a}) (e.g., CH); R² is hydrogen; R³ is halo (e.g., F); m is 1; and n is 1. In some embodiments, the compound of Formula (I) is Compound 401, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-ethyl piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 402, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L¹ and L² are absent; Y is C(R^{6a}) (e.g., CH); R² is hydrogen; R³ is methyl sulfonyl; m is 1; and n is 1. In some embodiments, the compound of Formula (I) is Compound 409, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 412, or a pharmaceutically acceptable salt, solvate, hydrate, tautomer, or stereoisomer thereof.

In some embodiments, for Formula (I), A is bicyclic heteroaryl (e.g., 7-chloro-2-methyl-2H-indazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 413, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 414, or a pharmaceutically acceptable salt, solvate, hydrate, tautomer, or stereoisomer thereof.

In some embodiments, for Formula (I), A is bicyclic heteroaryl (e.g., 2,7-dimethyl-2H-indazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 415, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-a]pyridinyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 416, or a pharmaceutically acceptable salt, solvate, hydrate, tautomer, or stereoisomer thereof.

In some embodiments, for Formula (I), A is bicyclic heteroaryl (e.g., 4-fluoro-2-methyl-1,3-benzoxazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 417, or a pharmaceutically acceptable salt, solvate, hydrate, tautomer, or stereoisomer thereof.

In some embodiments, for Formula (I), A is bicyclic heteroaryl (e.g., 2,4-dimethylbenzoxazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 418, or a pharmaceutically acceptable salt, solvate, hydrate, tautomer, or stereoisomer thereof.

In some embodiments, for Formula (I), A is bicyclic heteroaryl (e.g., 6-hydroxy-2,4-dimethyl-2H-indazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 419, or a pharmaceutically acceptable salt, solvate, hydrate, tautomer, or stereoisomer thereof.

In some embodiments, for Formula (I), A is bicyclic heteroaryl (e.g., 7-fluoro-6-hydroxy-2-methyl-2H-indazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 420, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 421, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; Y is N; R² is hydrogen; R³ is methyl; m is 1; and n is 1. In some embodiments, the compound of Formula (I) is Compound 423, or a pharmaceutically acceptable salt, solvate, hydrate, tautomer, or stereoisomer thereof.

In some embodiments, for Formula (I), A is bicyclic heteroaryl (e.g., 2-methylimidazo[1,2-a]pyrazinyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 428, or a pharmaceutically acceptable salt, solvate, hydrate, tautomer, or stereoisomer thereof.

In some embodiments, for Formula (I), A is bicyclic heteroaryl (e.g., 1,2,4-trimethyl-1H-benzimidazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 429, or a pharmaceutically acceptable salt, solvate, hydrate, tautomer, or stereoisomer thereof.

In some embodiments, for Formula (I), A is bicyclic heteroaryl (e.g., 4-fluoro-1,2-dimethyl-1H-benzimidazolyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 430, or a pharmaceutically acceptable salt, solvate, hydrate, tautomer, or stereoisomer thereof.

In some embodiments, for Formula (I), A is bicyclic heteroaryl (e.g., 2,7-dimethylpyrazolo[4,3-b]pyridinyl); B is bicyclic heterocyclyl (e.g., 4-azaspiro[2.5]octanyl); L¹ and L² are absent; Y is C(R^{6a}) (e.g., CH); R² is hydrogen; R³ is halo (e.g., F); m is 1; and n is 1. In some embodiments, the compound of Formula (I) is Compound 431, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., pyrrolidinyl); L¹ and L² are absent; Y is C(R^{6a}) (e.g., CH); R² is hydrogen; R³ is hydroxy; m is 1; and n is 1. In some embodiments, the compound of Formula (I) is Compound 432, or a pharmaceutically acceptable salt, solvate, hydrate, tautomer, or stereoisomer thereof.

In some embodiments, for Formula (I), A is bicyclic heteroaryl (e.g., 8-cyano-2-methylimidazo[1,2-a]pyridinyl); B is monocyclic heterocyclyl (e.g., pyrrolidinyl); L¹ and L² are absent; Y is C(R^{6a}) (e.g., CH); R² is hydrogen; R³ is hydroxy; m is 1; and n is 1. In some embodiments, the compound of Formula (I) is Compound 433, or a pharmaceutically acceptable salt, solvate, hydrate, tautomer, or stereoisomer thereof.

In some embodiments, for Formula (I), A is bicyclic heterocyclyl (e.g., 8-cyano-2-methylimidazo[1,2-a]pyridinyl); B is monocyclic heterocyclyl (e.g., pyrrolidinyl); L¹ and L² are absent; Y is C(R^{6a}) (e.g., CH); R² is hydrogen; R³ is halo (e.g., F); m is 1; and n is 1. In some embodiments, the compound of Formula (I) is Compound 434, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L¹ and L² are absent; Y is C(R^{6a}) (e.g., CH); R² is methyl; R³ is hydrogen; m is 1; and n is 1. In some embodiments, the compound of Formula (I) is Compound 440, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., pyrrolidinyl); L¹ and L² are absent; Y is C(R^{6a}) (e.g., CH); R² is methyl; R³ is hydrogen; m is 1; and n is 1. In some embodiments, the compound of Formula (I) is Compound 441, or a pharmaceutically acceptable salt, solvate, hydrate, tautomer, or stereoisomer thereof.

In some embodiments, for Formula (I), A is bicyclic heteroaryl (e.g., 8-cyano-2-methylimidazo[1,2-a]pyridinyl); B is monocyclic heterocyclyl (e.g., pyrrolidinyl); L¹ and L² are absent; Y is C(R^{6a}) (e.g., CH); R² is methyl; R³ is hydrogen; m is 1; and n is 1. In some embodiments, the compound of Formula (I) is Compound 442, or a pharmaceutically acceptable salt, solvate, hydrate, tautomer, or stereoisomer thereof.

In some embodiments, for Formula (V), A is bicyclic heteroaryl (e.g., 8-cyano-2-methylimidazo[1,2-a]pyridinyl); B is monocyclic heterocyclyl (e.g., pyrrolidinyl); L¹ and L² are absent; Y is C(R^{6a}) (e.g., CH₂); R² is hydrogen; R³ is hydroxyl; m is 1; and n is 2. In some embodiments, the compound of Formula (I) is Compound 443, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazinyl); B is bicyclic heterocyclyl (e.g., 4-azaspiro[2.5]octanyl); L¹ and L² are absent; Y is C(R^{6a}) (e.g., CH); R² is hydrogen; R³ is hydrogen; m is 1; and n is 1. In some embodiments, the compound of Formula (I) is Compound 444, Compound 445, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazinyl); B is bicyclic heterocyclyl (e.g., 4-azaspiro[2.5]octanyl); L¹ and L² are absent; Y is N; R² is hydrogen; R³ is halo (e.g., F); m is 1; and n is 1. In some embodiments, the compound of Formula (I) is Compound 446, Compound 447, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., piperidinyl); L¹ and L² are absent; Y is C(R^{6a}) (e.g., CH); R² is hydrogen; R³ is hydroxy; m is 1; and n is 1. In some embodiments, the compound of Formula (I) is Compound 448, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is C(R^{6a}) (e.g., CH); R² is hydrogen; R³ is methoxy; m is 1; and n is 1. In some embodiments, the compound of Formula (I) is Compound 449, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., pyrrolidinyl); L¹ and L² are absent; Y is C(R^{6a}) (e.g., CH); R² is methyl; R³ is hydrogen; m is 1; and n is 1. In some embodiments, the compound of Formula (I) is Compound 451, Compound 452, or a pharmaceutically acceptable salt, solvate, hydrate, tautomer, or stereoisomer thereof.

In some embodiments, for Formula (I), A is bicyclic heteroaryl (e.g., 2-methyl-5,6,7,8-tetrahydroimidazo[1,2-a]pyridinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 453 or a pharmaceutically acceptable salt, solvate, hydrate, tautomer, or stereoisomer thereof.

In some embodiments, for Formula (I), A is monocyclic heterocyclyl (e.g. 2,6-dimethyl piperazinyl); B is bicyclic heteroaryl (e.g., 2,8-dimethylimidazo[1,2-a]pyrazinyl); L¹ and L² are absent; Y is C(R^{6a}) (e.g., CH); R² is hydrogen; R³ is hydrogen; m is 1; and n is 1. In some embodiments, the compound of Formula (I) is Compound 454, 580, 581, 582, 583 or a pharmaceutically acceptable salt, solvate, hydrate, tautomer, or stereoisomer thereof.

In some embodiments, for Formula (I), A is bicyclic heteroaryl (e.g., 8-cyano-2-methylimidazo[1,2-a]pyridinyl); B is monocyclic heterocyclyl (e.g., pyrrolidinyl); L¹ and L² are absent; Y is C(R^{6a}) (e.g., CH); R² is methyl; R³ is hydrogen; m is 1; and n is 1. In some embodiments, the compound of Formula (I) is Compound 456, Compound 457, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L¹ and L² are absent; Y is C(R^{6a}) (e.g., CH); R² is methyl; R³ is halogen (e.g., F); m is 1; and n is 1. In some embodiments, the compound of Formula (I) is Compound 461, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., pyrrolidinyl); L¹ and L² are absent; Y is C(R^{6a}) (e.g., CH); R² is methyl; R³ is hydroxy; m is 1; and n is 1. In some embodiments, the compound of Formula (I) is Compound 462, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; Y is C(R^{6a}) (e.g., CH); R² is hydrogen; R³ is hydroxyl; m is 1; and n is 1. In some embodiments, the compound of Formula (I) is Compound 466 or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; Y is N; R² is hydrogen; R³ is methyl; m is 1; and n is 1. In some embodiments, the compound of Formula (I) is Compound 467, Compound 468, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; Y is N; R² is hydrogen; R³ is hydroxy; m is 1; and n is 1. In some embodiments, the compound of Formula (I) is Compound 469, Compound 470, 584, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is bicyclic heterocyclyl amino (e.g., 4-azaspiro[2.5]octanyl); L¹ and L² are absent; Y is C(R^{6a}) (e.g., CH); R² is hydrogen; R³ is hydrogen; m is 1; and n is 1. In some embodiments, the compound of Formula (I) is Compound 471, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is secondary amino (e.g., ethylmethylamino); L¹ and L² are absent; Y is N; R² is hydrogen; R³ is hydrogen; m is 1; and n is 1. In some embodiments, the compound of Formula (I) is Compound 472, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is secondary amino (e.g., N,N-dimethylethylamino); L¹ and L² are absent; Y is N; R² is hydrogen; R³ is hydrogen; m is 1; and n is 1. In some embodiments, the compound of Formula (I) is Compound 473, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L¹ and L² are absent; Y is C(R^{6a}) (e.g., CH); R² is hydrogen; R³ is fluoro; m is 1; and n is 1. In some embodiments, the compound of Formula (I) is Compound 474, or a pharmaceutically acceptable salt, solvate, hydrate, tautomer, or stereoisomer thereof.

In some embodiments, for Formula (I), A is bicyclic heteroaryl (e.g., 8-cyano-2-methylimidazo[1,2-a]pyridinyl); B is monocyclic heterocyclyl (e.g., piperidinyl); L¹ and L² are absent; Y is C(R^{6a}) (e.g., CH); R² is hydrogen; R³ is fluoro; m is 1; and n is 1. In some embodiments, the compound of Formula (I) is Compound 475, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is C(R^{6a}) (e.g., CH); R² is hydrogen; R³ is fluoro; m is 1; and n is 1. In some embodiments, the compound of Formula (I) is Compound 476, or a pharmaceutically acceptable salt, solvate, hydrate, tautomer, or stereoisomer thereof.

In some embodiments, for Formula (I), A is bicyclic heteroaryl (e.g., 7-fluoro-6-hydroxy-2-methyl-2H-indazolyl); B is monocyclic heterocyclyl (e.g., piperidinyl); L¹ and L² are absent; Y is C(R^{6a}) (e.g., CH); R² is hydrogen; R³ is fluoro; m is 1; and n is 1. In some embodiments, the compound of Formula (I) is Compound 477, or a pharmaceutically acceptable salt, solvate, hydrate, tautomer, or stereoisomer thereof.

In some embodiments, for Formula (I), A is bicyclic heteroaryl (e.g., 2,7-dimethyl-6-hydroxy-2H-indazolyl); B is monocyclic heterocyclyl (e.g., piperidinyl); L¹ and L² are absent; Y is C(R^{6a}) (e.g., CH); R² is hydrogen; R³ is fluoro; m is 1; and n is 1. In some embodiments, the compound of Formula (I) is Compound 478, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., piperidinyl); L¹ and L² are absent; Y is C(R^{6a}) (e.g., CH); R² is hydrogen; R³ is fluoro; m is 1; and n is 1. In some embodiments, the compound of Formula (I) is Compound 479, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; Y is C(R^{6a}) (e.g., CH); R² is hydrogen; R³ is fluoro; m is 1; and n is 1. In some embodiments, the compound of Formula (I) is Compound 480, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., pyrrolidinyl); L¹ and L² are absent; Y is C(R^{6a}) (e.g., CH); R² is hydrogen; R³ is fluoro; m is 1; and n is 1. In some embodiments, the compound of Formula (I) is Compound 481, or a pharmaceutically acceptable salt, solvate, hydrate, tautomer, or stereoisomer thereof.

In some embodiments, for Formula (I), A is bicyclic heteroaryl (e.g., 2-methyl-2,4,6,7-tetrahydro-5-pyrazolo[4,3-c]pyridinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 509, or a pharmaceutically acceptable salt, solvate, hydrate, tautomer, or stereoisomer thereof.

In some embodiments, for Formula (I), A is bicyclic heteroaryl (e.g., 8-chloro-2-methylimidazo[1,2-a]pyridinyl); B is bicyclic heterocyclyl amino (e.g., 4-azaspiro[2.5]octanyl); L¹ and L² are absent; Y is N; R² is hydrogen; R³ is hydrogen; m is 1; and n is 1. In some embodiments, the compound of Formula (I) is Compound 510, or a pharmaceutically acceptable salt, solvate, hydrate, tautomer, or stereoisomer thereof.

In some embodiments, for Formula (I), A is bicyclic heteroaryl (e.g., 2-methyl-2H-pyrazolo[4,3-b]pyridinyl); B is bicyclic heterocyclyl amino (e.g., 4-azaspiro[2.5]octanyl); L¹ and L² are absent; Y is N; R² is hydrogen; R³ is hydrogen; m is 1; and n is 1. In some embodiments, the compound of Formula (I) is Compound 511, or a pharmaceutically acceptable salt, solvate, hydrate, tautomer, or stereoisomer thereof.

In some embodiments, for Formula (I), A is bicyclic heteroaryl (e.g., 2,7-dimethyl-2H-indazolyl); B is bicyclic heterocyclyl amino (e.g., 4-azaspiro[2.5]octanyl); L¹ and L² are absent; Y is N; R² is hydrogen; R³ is hydrogen; m is 1; and n is 1. In some embodiments, the compound of Formula (I) is Compound 512, or a pharmaceutically acceptable salt, solvate, hydrate, tautomer, or stereoisomer thereof.

In some embodiments, for Formula (I), A is bicyclic heteroaryl (e.g., 2,4-dimethylbenzo[d]oxazolyl); B is bicyclic heterocyclyl amino (e.g., 4-azaspiro[2.5]octanyl); L¹ and L² are absent; Y is N; R² is hydrogen; R³ is hydrogen; m is 1; and n is 1. In some embodiments, the compound of Formula (I) is Compound 513, or a pharmaceutically acceptable salt, solvate, hydrate, tautomer, or stereoisomer thereof.

In some embodiments, for Formula (I), A is bicyclic heteroaryl (e.g., 8-methoxy-2-methylimidazo[1,2-b]pyridazyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 514, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-(1-fluoro)-ethylpiperidinyl); L¹ and L² are absent; Y is N; R² is hydrogen; R³ is hydrogen; m is 1; and n is 1. In some embodiments, the compound of Formula (I) is Compound 515, or a pharmaceutically acceptable salt, solvate, hydrate, tautomer, or stereoisomer thereof.

In some embodiments, for Formula (I), A is bicyclic heteroaryl (e.g., 2-methylimidazo[1,2-b]pyridazyl-8-carboxylic acid); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 516, or a pharmaceutically acceptable salt, solvate, hydrate, tautomer, or stereoisomer thereof.

In some embodiments, for Formula (I), A is bicyclic heteroaryl (e.g., 2-methylimidazo[1,2-b]pyridazyl-8-carboxylate); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 517, or a pharmaceutically acceptable salt, solvate, hydrate, tautomer, or stereoisomer thereof.

In some embodiments, for Formula (I), A is bicyclic heteroaryl (e.g., N,2-methylimidazo[1,2-b]pyridazyl-8-carboxamide); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 518, or a pharmaceutically acceptable salt, solvate, hydrate, tautomer, or stereoisomer thereof.

In some embodiments, for Formula (I), A is bicyclic heteroaryl (e.g., 2-methyl-8-phenoxyimidazo[1,2-b]pyridazinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 519, or a pharmaceutically acceptable salt, solvate, hydrate, tautomer, or stereoisomer thereof.

In some embodiments, for Formula (I), A is bicyclic heteroaryl (e.g., 2-(2-methylimidazo[1,2-b]pyridazin-8-yl)acetonitrile); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 520, or a pharmaceutically acceptable salt, solvate, hydrate, tautomer, or stereoisomer thereof.

In some embodiments, for Formula (I), A is bicyclic heteroaryl (e.g., 5-methyl-1-pyrazolo[4,5-c]pyridin-4(5H)-one); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 521, 522, or a pharmaceutically acceptable salt, solvate, hydrate, tautomer, or stereoisomer thereof.

In some embodiments, for Formula (I), A is bicyclic heteroaryl (e.g., 2-methylimidazo[1,2-b]pyridazyl-8-carboxamide); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 523, or a pharmaceutically acceptable salt, solvate, hydrate, tautomer, or stereoisomer thereof.

In some embodiments, for Formula (I), A is bicyclic heteroaryl (e.g., N,N,2-methylimidazo[1,2-b]pyridazyl-8-carboxamide); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 524, or a pharmaceutically acceptable salt, solvate, hydrate, tautomer, or stereoisomer thereof.

In some embodiments, for Formula (I), A is bicyclic heteroaryl (e.g., (2-methylimidazo[1,2-b]pyridazin-8-yl)methanol); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 525, or a pharmaceutically acceptable salt, solvate, hydrate, tautomer, or stereoisomer thereof.

In some embodiments, for Formula (I), A is bicyclic heteroaryl (e.g., (8-(benzyloxy)-2-methylimidazo[1,2-b]pyridazinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 526, or a pharmaceutically acceptable salt, solvate, hydrate, tautomer, or stereoisomer thereof.

In some embodiments, for Formula (I), A is bicyclic heteroaryl (e.g., (methyl 2-(2-methylimidazo[1,2-b]pyridazin-8-yl)acetate); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 527, or a pharmaceutically acceptable salt, solvate, hydrate, tautomer, or stereoisomer thereof.

In some embodiments, for Formula (I), A is bicyclic heteroaryl (e.g., (2-(2-methylimidazo[1,2-b]pyridazin-8-yl)acetamide); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 528, or a pharmaceutically acceptable salt, solvate, hydrate, tautomer, or stereoisomer thereof.

In some embodiments, for Formula (I), A is bicyclic heteroaryl (e.g., ethyl 2-methylimidazo[1,2-b]pyridazine-8-carboxylate); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 529, or a pharmaceutically acceptable salt, solvate, hydrate, tautomer, or stereoisomer thereof.

In some embodiments, for Formula (I), A is bicyclic heteroaryl (e.g., 2-methyl-5-pyrazolo[4,3-c]pyridin-4(2H)-one); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 530, or a pharmaceutically acceptable salt, solvate, hydrate, tautomer, or stereoisomer thereof.

In some embodiments, for Formula (I), A is bicyclic heteroaryl (e.g., 2-(2-methylimidazo[1,2-b]pyridazin-8-yl)acetic acid); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 531, or a pharmaceutically acceptable salt, solvate, hydrate, tautomer, or stereoisomer thereof.

In some embodiments, for Formula (I), A is bicyclic heteroaryl (e.g., N,N-dimethyl-2-(2-methylimidazo[1,2-b]pyridazin-8-yl)acetamide); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 532, or a pharmaceutically acceptable salt, solvate, hydrate, tautomer, or stereoisomer thereof.

In some embodiments, for Formula (I), A is bicyclic heteroaryl (e.g., 2-(2-methylimidazo[1,2-b]pyridazin-8-yl)ethan-1-ol); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 533, or a pharmaceutically acceptable salt, solvate, hydrate, tautomer, or stereoisomer thereof.

In some embodiments, for Formula (I), A is bicyclic heteroaryl (e.g., N-methyl-2-(2-methylimidazo[1,2-b]pyridazin-8-yl)acetamide); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 534, or a pharmaceutically acceptable salt, solvate, hydrate, tautomer, or stereoisomer thereof.

In some embodiments, for Formula (I), A is bicyclic heteroaryl (e.g., 8-((4H-1,2,4-triazol-3-yl)methyl)-2-methylimidazo[1,2-b]pyridazinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 535, or a pharmaceutically acceptable salt, solvate, hydrate, tautomer, or stereoisomer thereof.

In some embodiments, for Formula (I), A is bicyclic heteroaryl (e.g., 8-((1H-tetrazol-5-yl)methyl)-2-methylimidazo[1,2-b]pyridazinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 536, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-ethylpiperidinyl); L¹ and L² are absent; Y is N; R² is hydrogen; R³ is fluoro; m is 1; and n is 1. In some embodiments, the compound of Formula (I) is Compound 537, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-(1-fluoro)-ethylpiperidinyl); L¹ and L² are absent; Y is N; R² is hydrogen; R³ is fluoro; m is 1; and n is 1. In some embodiments, the compound of Formula (I) is Compound 538, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-methylpiperidinyl); L¹ and L² are absent; Y is N; R² is hydrogen; R³ is fluoro; m is 1; and n is 1. In some embodiments, the compound of Formula (I) is Compound 539, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is N; R² is hydrogen; R³ is fluoro; m is 1; and n is 1. In some embodiments, the compound of Formula (I) is Compound 540, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is N; R² is hydrogen; R³ is hydroxy; m is 1; and n is 1. In some embodiments, the compound of Formula (I) is Compound 541, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-ethylpiperidinyl); L¹ and L² are absent; Y is N; R² is hydrogen; R³ is hydroxy; m is 1; and n is 1. In some embodiments, the compound of Formula (I) is Compound 542, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-methylpiperidinyl); L¹ and L² are absent; Y is N; R² is hydrogen; R³ is hydroxy; m is 1; and n is 1. In some embodiments, the compound of Formula (I) is Compound 543, or a pharmaceutically acceptable salt, solvate, hydrate, tautomer, or stereoisomer thereof.

In some embodiments, for Formula (I), A is bicyclic heteroaryl (e.g., 8-(2-fluorophenoxy)-2-methylimidazo[1,2-b]pyridazinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 544, or a pharmaceutically acceptable salt, solvate, hydrate, tautomer, or stereoisomer thereof.

In some embodiments, for Formula (I), A is bicyclic heteroaryl (e.g., 8-(3-fluorophenoxy)-2-methylimidazo[1,2-b]pyridazinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 545, or a pharmaceutically acceptable salt, solvate, hydrate, tautomer, or stereoisomer thereof.

In some embodiments, for Formula (I), A is bicyclic heteroaryl (e.g., 8-(4-fluorophenoxy)-2-methylimidazo[1,2-b]pyridazinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 546, or a pharmaceutically acceptable salt, solvate, hydrate, tautomer, or stereoisomer thereof.

In some embodiments, for Formula (I), A is bicyclic heteroaryl (e.g., 8-(3-methoxyphenoxy)-2-methylimidazo[1,2-b]pyridazinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 547, or a pharmaceutically acceptable salt, solvate, hydrate, tautomer, or stereoisomer thereof.

In some embodiments, for Formula (I), A is bicyclic heteroaryl (e.g., 8-phenoxy-2-methylimidazo[1,2-b]pyridazinyl); B is monocyclic heterocyclyl (e.g., piperidinyl); L¹ and L² are absent; Y is N; R² is hydrogen; R³ is fluoro; m is 1; and n is 1. In some embodiments, the compound of Formula (I) is Compound 548, or a pharmaceutically acceptable salt, solvate, hydrate, tautomer, or stereoisomer thereof.

In some embodiments, for Formula (I), A is bicyclic heteroaryl (e.g., 8-phenoxy-2-methylimidazo[1,2-b]pyridazinyl); B is monocyclic heterocyclyl (e.g., piperidinyl); L¹ and L² are absent; Y is N; R² is hydrogen; R³ is hydrogen; m is 1; and n is 1. In some embodiments, the compound of Formula (I) is Compound 549, or a pharmaceutically acceptable salt, solvate, hydrate, tautomer, or stereoisomer thereof.

In some embodiments, for Formula (I), A is bicyclic heteroaryl (e.g., 2-methyl-8-(pyridin-3-yloxy)imidazo[1,2-b]pyridazinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 550, or a pharmaceutically acceptable salt, solvate, hydrate, tautomer, or stereoisomer thereof.

In some embodiments, for Formula (I), A is bicyclic heteroaryl (e.g., 1-(2-methylimidazo[1,2-b]pyridazin-8-yl)pyridin-4(1H)-one); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 551, or a pharmaceutically acceptable salt, solvate, hydrate, tautomer, or stereoisomer thereof.

In some embodiments, for Formula (I), A is bicyclic heteroaryl (e.g., 1-(2-methylimidazo[1,2-b]pyridazin-8-yl)pyridin-2(1H)-one); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 552, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., N-(1-fluoro)-ethylpiperidinyl); L¹ and L² are absent; Y is N; R² is hydrogen; R³ is hydroxy; m is 1; and n is 1. In some embodiments, the compound of Formula (I) is Compound 553, or a pharmaceutically acceptable salt, solvate, hydrate, tautomer, or stereoisomer thereof.

In some embodiments, for Formula (I), A is bicyclic heteroaryl (e.g., 2,7-dimethyl-2H-indazolyl); B is bicyclic heterocyclyl (e.g., 4-azaspiro[2.5]octanyl); L¹ and L² are absent; Y is N; R² is hydrogen; R³ is fluoro; m is 1; and n is 1. In some embodiments, the compound of Formula (I) is Compound 554, or a pharmaceutically acceptable salt, solvate, hydrate, tautomer, or stereoisomer thereof.

In some embodiments, for Formula (I), A is bicyclic heteroaryl (e.g., 2,4-dimethylbenzo[d]oxazolyl); B is bicyclic heterocyclyl (e.g., 4-azaspiro[2.5]octanyl); L¹ and L² are absent; Y is N; R² is hydrogen; R³ is fluoro; m is 1; and n is 1. In some embodiments, the compound of Formula (I) is Compound 555, or a pharmaceutically acceptable salt, solvate, hydrate, tautomer, or stereoisomer thereof.

In some embodiments, for Formula (I), A is bicyclic heteroaryl (e.g., 8-chloro-2-methylimidazo[1,2-a]pyridinyl); B is bicyclic heterocyclyl (e.g., 4-azaspiro[2.5]octanyl); L¹ and L² are absent; Y is N; R² is hydrogen; R³ is fluoro; m is 1; and n is 1. In some embodiments, the compound of Formula (I) is Compound 556, or a pharmaceutically acceptable salt, solvate, hydrate, tautomer, or stereoisomer thereof.

In some embodiments, for Formula (I), A is bicyclic heteroaryl (e.g., 8-chloro-2-methylimidazo[1,2-a]pyridinyl); B is bicyclic heterocyclyl (e.g., 4-azaspiro[2.5]octanyl); L¹ and L² are absent; Y is N; R² is hydrogen; R³ is hydroxy; m is 1; and n is 1. In some embodiments, the compound of Formula (I) is Compound 557, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L¹ and L² are absent; Y is C(R^{6a}) (e.g., CH); R² is ethyl; R³ is fluoro; m is 1; and n is 1. In some embodiments, the compound of Formula (I) is Compound 558, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperidinyl); L¹ and L² are absent; Y is C(R^{6a}) (e.g., CH); R² is vinyl; R³ is fluoro; m is 1; and n is 1. In some embodiments, the compound of Formula (I) is Compound 559, or a pharmaceutically acceptable salt, solvate, hydrate, tautomer, or stereoisomer thereof.

In some embodiments, for Formula (I), A is bicyclic heteroaryl (e.g., 8-cyano-2-methylimidazo[1,2-a]pyridinyl); B is monocyclic heterocyclyl (e.g., piperidinyl); L¹ and L² are absent; Y is C(R^{6a}) (e.g., CH); R² is hydrogen; R³ is hydrogen; m is 1; and n is 1. In some embodiments, the compound of Formula (I) is Compound 560, or a pharmaceutically acceptable salt, solvate, hydrate, tautomer, or stereoisomer thereof.

In some embodiments, for Formula (I), A is bicyclic heteroaryl (e.g., 2,7-dimethyl-2H-indazolyl); B is bicyclic heterocyclyl (e.g., 4-azaspiro[2.5]octanyl); L¹ and L² are absent; Y is N; R² is hydrogen; R³ is hydroxy; m is 1; and n is 1. In some embodiments, the compound of Formula (I) is Compound 561, or a pharmaceutically acceptable salt, solvate, hydrate, tautomer, or stereoisomer thereof.

In some embodiments, for Formula (I), A is bicyclic heteroaryl (e.g., 2-methyl-2H-pyrazolo[4,3-b]pyridinyl); B is bicyclic heterocyclyl (e.g., 4-azaspiro[2.5]octanyl); L¹ and L² are absent; Y is N; R² is hydrogen; R³ is hydroxy; m is 1; and n is 1. In some embodiments, the compound of Formula (I) is Compound 562, or a pharmaceutically acceptable salt, solvate, hydrate, tautomer, or stereoisomer thereof.

In some embodiments, for Formula (I), A is bicyclic heteroaryl (e.g., 2-methyl-2H-pyrazolo[4,3-b]pyridinyl); B is bicyclic heterocyclyl (e.g., 4-azaspiro[2.5]octanyl); L¹ and L² are absent; Y is N; R² is hydrogen; R³ is fluoro; m is 1; and n is 1. In some embodiments, the compound of Formula (I) is Compound 563, or a pharmaceutically acceptable salt, solvate, hydrate, tautomer, or stereoisomer thereof.

In some embodiments, for Formula (I), A is bicyclic heteroaryl (e.g., 2,4-dimethylbenzo[d]oxazolyl); B is bicyclic heterocyclyl (e.g., 4-azaspiro[2.5]octanyl); L¹ and L² are absent; Y is N; R² is hydrogen; R³ is hydroxy; m is 1; and n is 1. In some embodiments, the compound of Formula (I) is Compound 564, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., N-methyl-4-azaspiro[2.5]octanyl); L¹ and L² are absent; Y is N; R² is hydrogen; R³ is fluoro; m is 1; and n is 1. In some embodiments, the compound of Formula (I) is Compound 565, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., piperidinyl); L¹ and L² are absent; Y is N; R² is hydrogen; R³ is amino; m is 1; and n is 1. In some embodiments, the compound of Formula (I) is Compound 566, or a pharmaceutically acceptable salt, solvate, hydrate, tautomer, or stereoisomer thereof.

In some embodiments, the compound of Formula (II) is a compound of Formula (II-a):

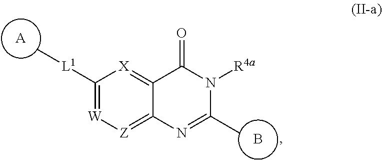

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of W, X, and Z is independently C(R³) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}, or —S(O)ₓR^{D}; R^{4a} is hydrogen, C₁-C₆-alkyl, C₁-C₆-heteroalkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

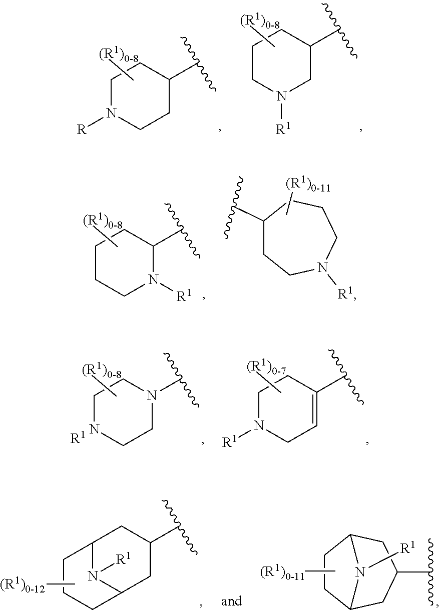

wherein R¹ is as defined herein. In some embodiments, A is selected from,

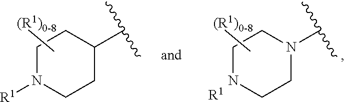

wherein R¹ is as defined herein. In some embodiments, A is selected from

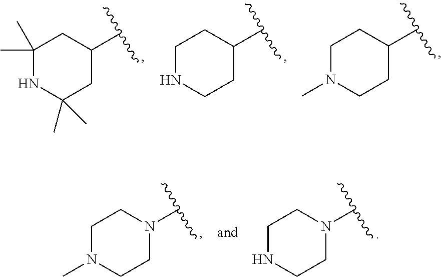

In some embodiments, A is heteroaryl. In some embodiments, A is a nitrogen-containing heteroaryl. In some embodiments, A is a bicyclic nitrogen-containing heteroaryl.

In some embodiments, A is selected from

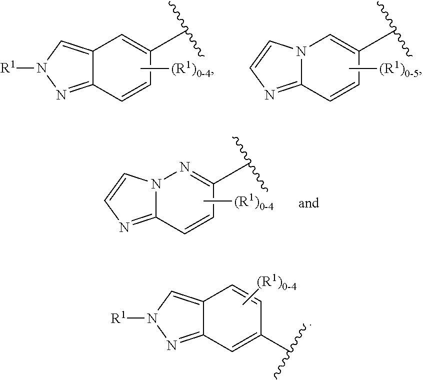

In some embodiments A is

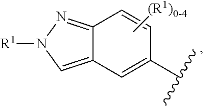

wherein R¹ is as defined herein. In some embodiments, A is selected from

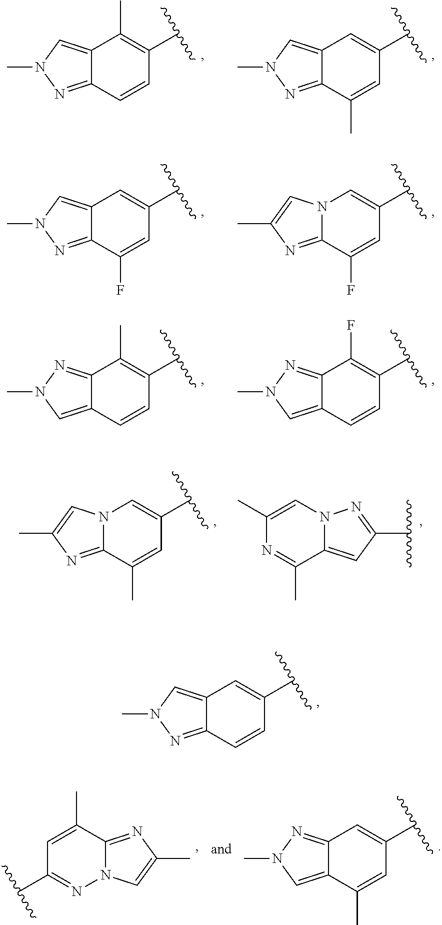

In some embodiments, A is

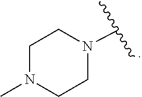 [SMILES: *N1CCN(C)CC1]

In some embodiments A is

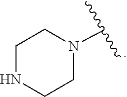 [SMILES: *N1CCNCC1]

In some embodiments, A is

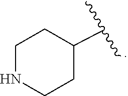 [SMILES: *C1CCNCC1]

In some embodiments, A is

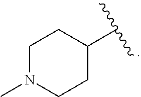 [SMILES: *C1CCN(C)CC1]

In some embodiments, A is

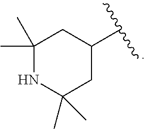 [SMILES: *C1CC(C)(C)NC(C)(C)C1]

In some embodiments, A is

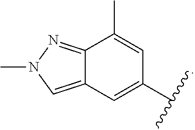 [SMILES: *c1cc(C)c2nn(C)cc2c1]

In some embodiments, A is

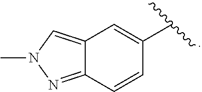 [SMILES: *c1ccc2nn(C)cc2c1]

In some embodiments, A is

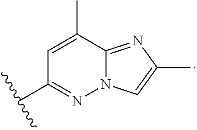 [SMILES: *c1cc(C)c2nc(C)cn2n1]

In some embodiments, A is

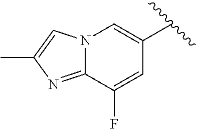 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

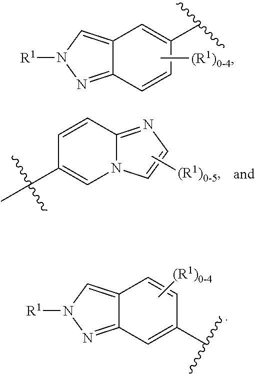

In some embodiments, B is

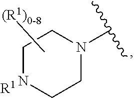

wherein R¹ is as defined herein.

In some embodiments, B is selected from

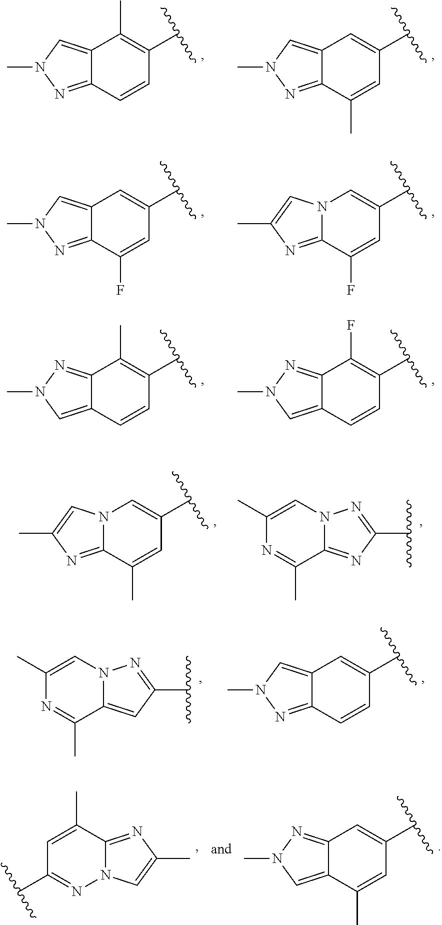

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

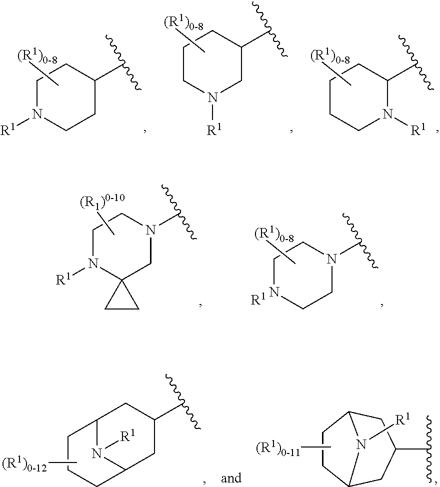

wherein R¹ is as defined herein. In some embodiments, B is selected from

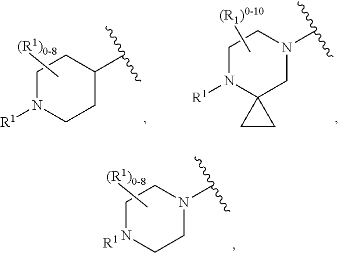

wherein R¹ is as defined herein. In some embodiments, B is selected from,

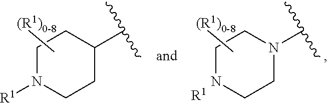

wherein R¹ is as defined herein.

In some embodiments, B is selected from

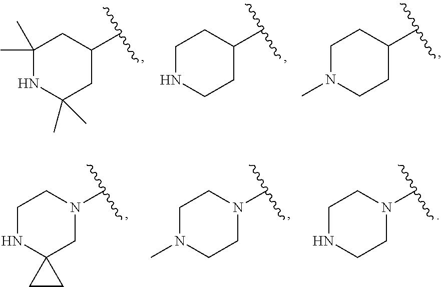

In some embodiments, B is

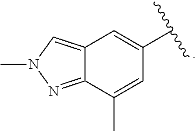 [SMILES: *c1cc(C)c2nn(C)cc2c1]

In some embodiments, B is

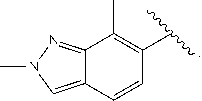 [SMILES: *c1ccc2cn(C)nc2c1C]

In some embodiments, B is

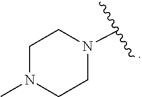 [SMILES: *N1CCN(C)CC1]

In some embodiments, B is

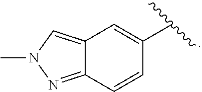 [SMILES: *c1ccc2nn(C)cc2c1]

In some embodiments, B is

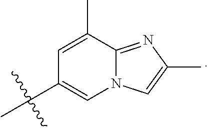 [SMILES: *c1cc(C)c2nc(C)cn2c1]

In some embodiments, B is

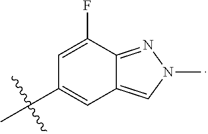 [SMILES: *c1cc(F)c2nn(C)cc2c1]

In some embodiments, B is

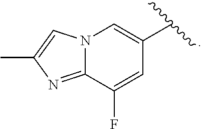 [SMILES: *c1cc(F)c2nc(C)cn2c1]

In some embodiments, B is

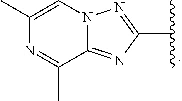 [SMILES: *c1nc2c(C)nc(C)cn2n1]

In some embodiments, B is

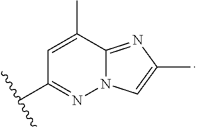 [SMILES: *c1cc(C)c2nc(C)cn2n1]

In some embodiments, B is

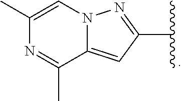 [SMILES: *c1cc2c(C)nc(C)cn2n1]

In some embodiments, B is

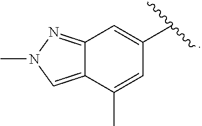 [SMILES: *c1cc(C)c2cn(C)nc2c1]

In some embodiments, B is

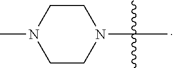 [SMILES: *N1CCN(C)CC1]

In some embodiments, B is

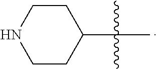 [SMILES: *C1CCNCC1]

In some embodiments, B is

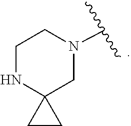 [SMILES: *N1CCNC2(CC2)C1]

In some embodiments, L¹ is absent or N(CH₃). In some embodiments, L¹ is absent. In some embodiments, L¹ is N(CH₃).

In some embodiments, each of W, X, and Z may independently be N or C(R³). In some embodiments, W is C(R³) (e.g., CH). In some embodiments, W is N. In some embodiments, X is C(R³) (e.g., CH). In some embodiments, X is N. In some embodiments, Z is C(R³) (e.g., CH). In some embodiments, Z is N. In some embodiments, each of W and X is independently C(R³) (e.g., CH). In some embodiments, each of W and Z is independently C(R³) (e.g., CH). In some embodiments, each of X and Z is independently C(R³) (e.g., CH). In some embodiments, each of W, X, and Z is independently C(R³) (e.g., CH).

In some embodiments, R^{4a} is hydrogen or C₁-C₆ alkyl. In some embodiments, R^{4a} is hydrogen.

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹.

In some embodiments, A is a bicyclic heteroaryl and B is a monocyclic heterocyclyl. In some embodiments of Formula (II), Z is N. In some embodiments of Formula (II), each of W, X, and Z is not independently C(R³), e.g., (CH).

In some embodiments, the compound of Formula (II) is a compound of Formula (II-b):

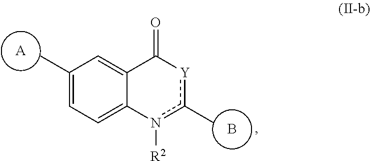

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; Y is N, N(R^{4a}), C(R^{4b}), or C(R^{4b})(R^{4c}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R^{4a} is hydrogen, C₁-C₆-alkyl, C₁-C₆-heteroalkyl, or C₁-C₆-haloalkyl; each of R^{4b} and R^{4c} is independently hydrogen, C₁-C₆-alkyl, C₁-C₆-heteroalkyl, C₁-C₆-haloalkyl, halo, or —OR^{A}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

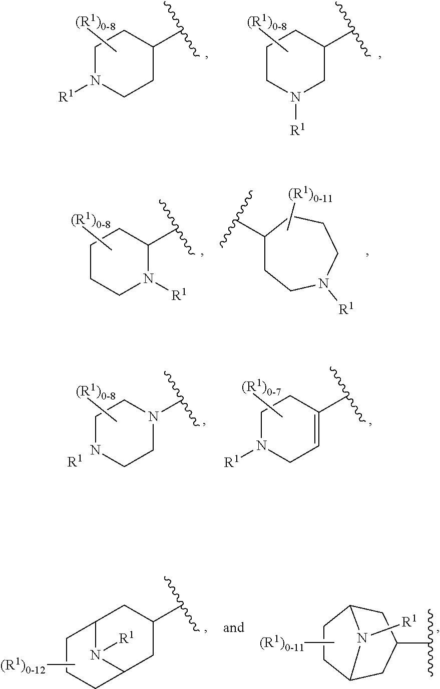

wherein R¹ is as defined herein.

In some embodiments, A is selected from

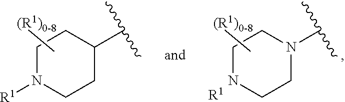

wherein R¹ is as defined herein.

In some embodiments, A is selected from H

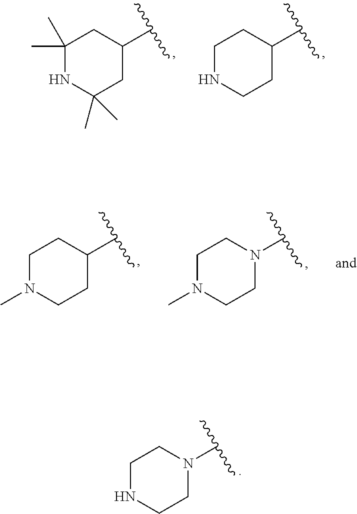

In some embodiments, A is heteroaryl. In some embodiments, A is a nitrogen-containing heteroaryl. In some embodiments, A is a bicyclic nitrogen-containing heteroaryl.

In some embodiments, A is selected from

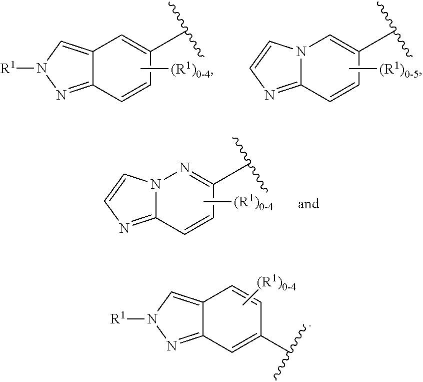

In some embodiments, A is

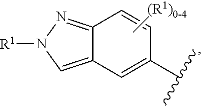

wherein R¹ is as defined herein. In some embodiments, A is selected from

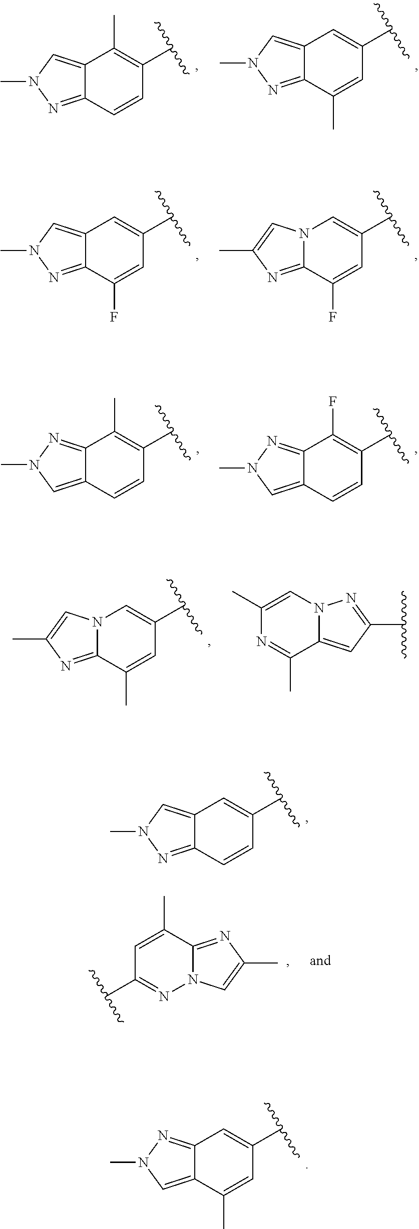

In some embodiments, A is

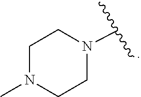 [SMILES: *N1CCN(C)CC1]

In some embodiments, A is

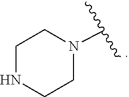 [SMILES: *N1CCNCC1]

In some embodiments, A is

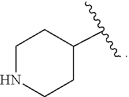 [SMILES: *C1CCNCC1]

In some embodiments, A is

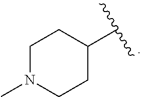 [SMILES: *C1CCN(C)CC1]

In some embodiments, A is

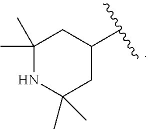 [SMILES: *C1CC(C)(C)NC(C)(C)C1]

In some embodiments, A is

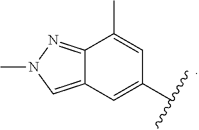 [SMILES: *c1cc(C)c2nn(C)cc2c1]

In some embodiments, A is

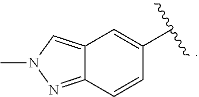 [SMILES: *c1ccc2nn(C)cc2c1]

In some embodiments, A is

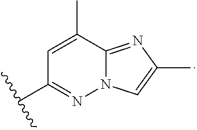 [SMILES: *c1cc(C)c2nc(C)cn2n1]

In some embodiments A is

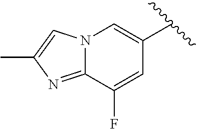 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

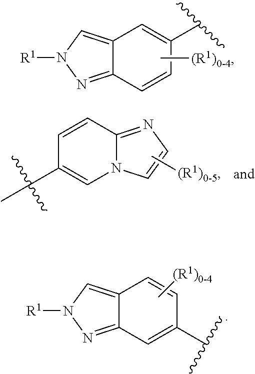

In some embodiments, B is

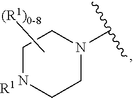

wherein R¹ is as defined herein.

In some embodiments, B is selected from

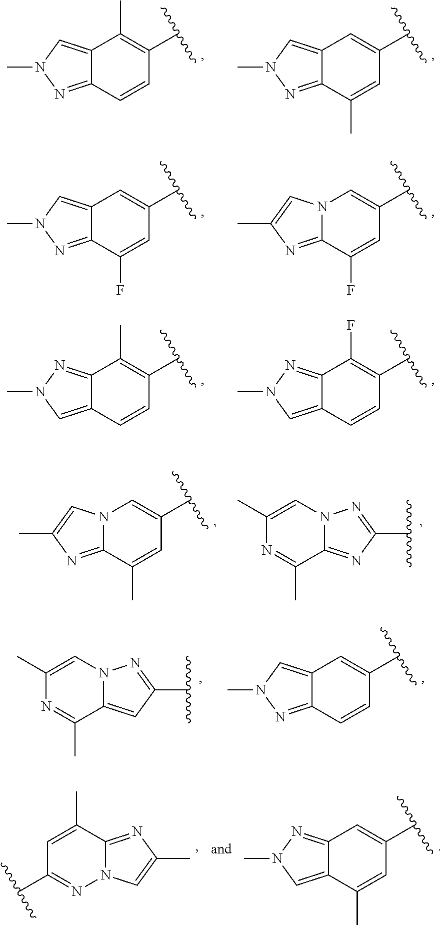

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

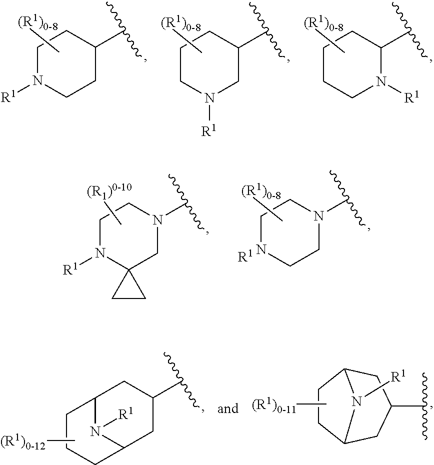

wherein R¹ is as defined herein. In some embodiments, B is selected from

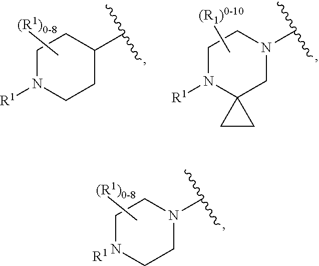

wherein R¹ is as defined herein. In some embodiments, B is selected from,

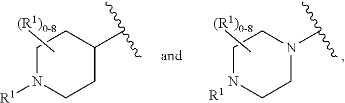

wherein R¹ is as defined herein.

In some embodiments, B is selected from

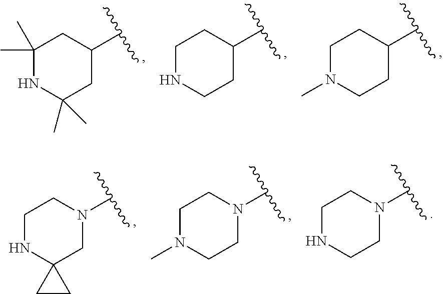

In some embodiments, B is

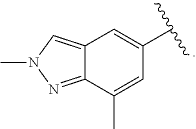 [SMILES: *c1cc(C)c2nn(C)cc2c1]

In some embodiments, B is

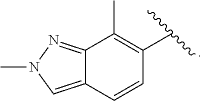 [SMILES: *c1ccc2cn(C)nc2c1C]

In some embodiments, B is

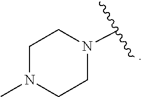 [SMILES: *N1CCN(C)CC1]

In some embodiments, B is

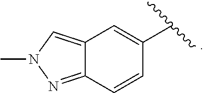 [SMILES: *c1ccc2nn(C)cc2c1]

In some embodiments, B is

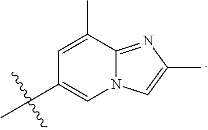 [SMILES: *c1cc(C)c2nc(C)cn2c1]

In some embodiments, B is

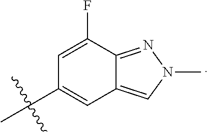 [SMILES: *c1cc(F)c2nn(C)cc2c1]

In some embodiments, B is

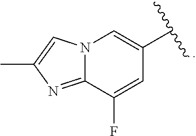 [SMILES: *c1cc(F)c2nc(C)cn2c1]

In some embodiments, B is

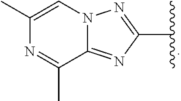 [SMILES: *c1nc2c(C)nc(C)cn2n1]

In some embodiments, B is

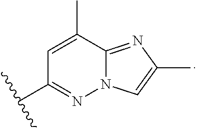 [SMILES: *c1cc(C)c2nc(C)cn2n1]

In some embodiments, B is

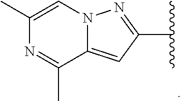 [SMILES: *c1cc2c(C)nc(C)cn2n1]

In some embodiments, B is

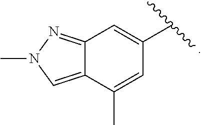 [SMILES: *c1cc(C)c2cn(C)nc2c1]

In some embodiments, B is

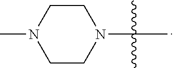 [SMILES: *N1CCN(C)CC1]

In some embodiments, B is

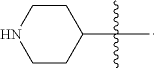 [SMILES: *C1CCNCC1]

In some embodiments, B is

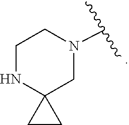 [SMILES: *N1CCNC2(CC2)C1]

As generally described, Y may be N, N(R^{4a}), C(R^{4b}), or C(R^{4b})(R^{4c}), wherein the dashed lines in the ring comprising Y may be single or double bonds as valency permits. In some embodiments, Y is N(R^{4a}) or C(R^{4b}). In some embodiments, Y is N(R^{4a}) (e.g., NH). In some embodiments, Y is C(R^{4b}) (e.g., CH).

In some embodiments, R² is absent.

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹. In some embodiments, A is a bicyclic heteroaryl and B is a monocyclic heterocyclyl.

In some embodiments, the compound of Formula (II) is a compound of Formula (II-c):

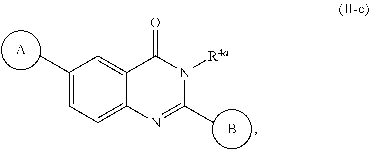

or a pharmaceutically acceptable salt, solvate, hydrate, tautomer, or stereoisomer thereof, wherein A is a monocyclic nitrogen-containing heterocyclyl optionally substituted with one or more R¹; B is a bicyclic nitrogen-containing heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; R^{4a} is hydrogen, C₁-C₆-alkyl, C₁-C₆-heteroalkyl, or C₁-C₆-haloalkyl; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

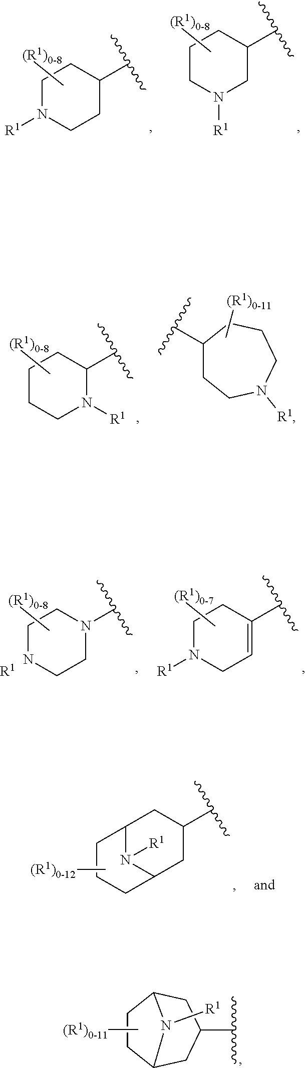

wherein R¹ is as defined herein.

In some embodiments, A is selected from,

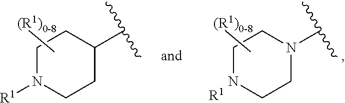

wherein R¹ is as defined herein.

In some embodiments, A is selected from

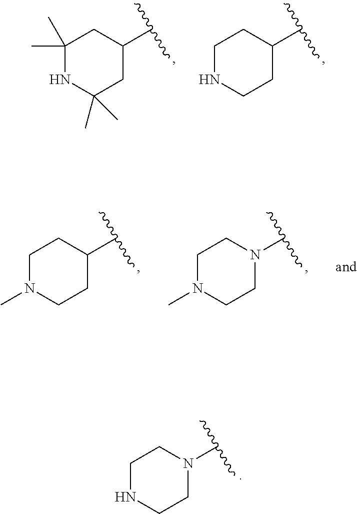

In some embodiments, A is heteroaryl. In some embodiments, A is a nitrogen-containing heteroaryl. In some embodiments, A is a bicyclic nitrogen-containing heteroaryl.

In some embodiments, A is selected from

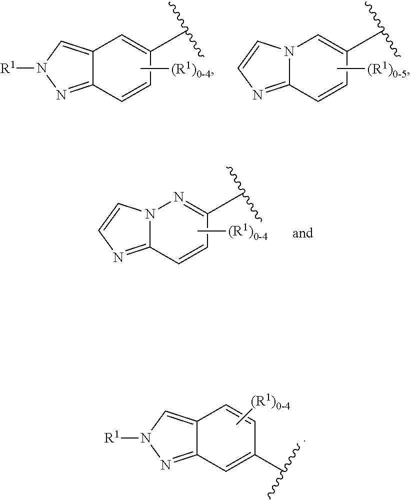

In some embodiments, A is

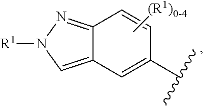

wherein R¹ is as defined herein. In some embodiments, A is selected from

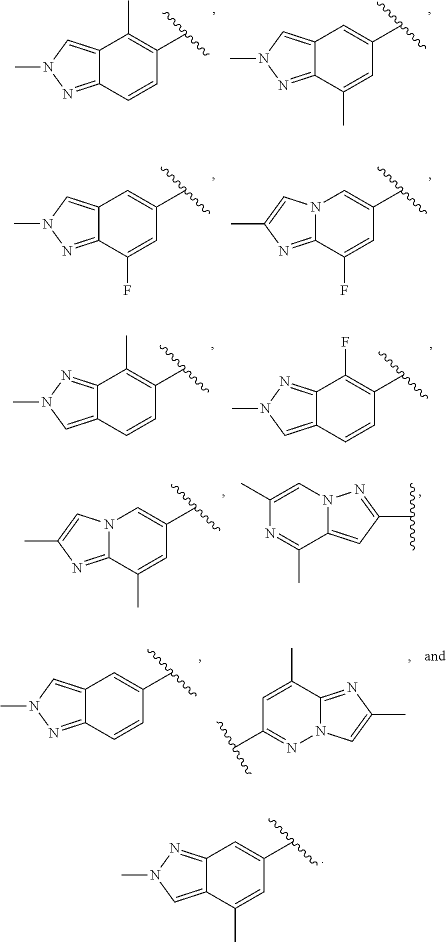

In some embodiments, A is

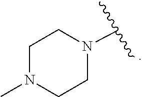 [SMILES: *N1CCN(C)CC1]

In some embodiments A is

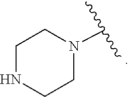 [SMILES: *N1CCNCC1]

In some embodiments, A is

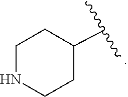 [SMILES: *C1CCNCC1]

In some embodiments, A is

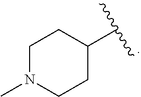 [SMILES: *C1CCN(C)CC1]

In some embodiments, A is

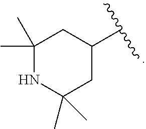 [SMILES: *C1CC(C)(C)NC(C)(C)C1]

In some embodiments, A is

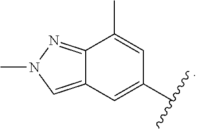 [SMILES: *c1cc(C)c2nn(C)cc2c1]

In some embodiments, A is

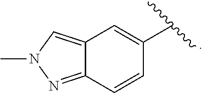 [SMILES: *c1ccc2nn(C)cc2c1]

In some embodiments, A is

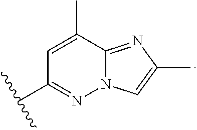 [SMILES: *c1cc(C)c2nc(C)cn2n1]

In some embodiments, A is

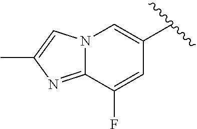 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

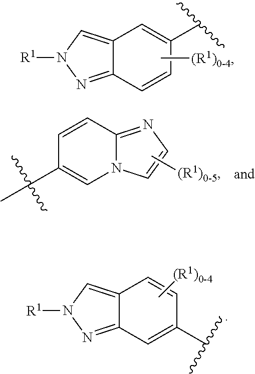

In some embodiments, B is

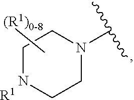

wherein R¹ is as defined herein.

In some embodiments, B is selected from

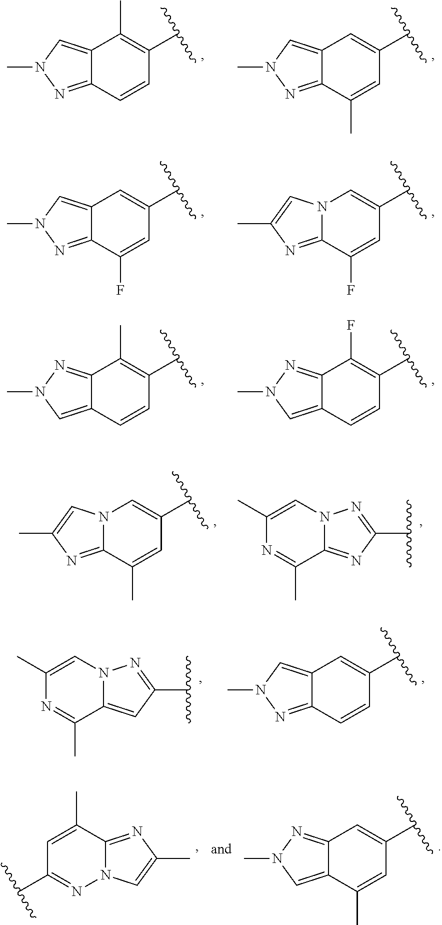

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

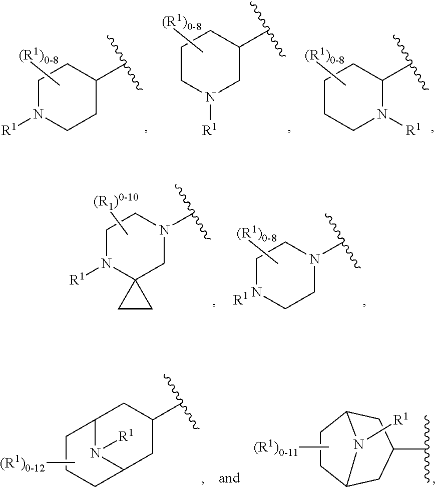

wherein R¹ is as defined herein. In some embodiments, B is selected from

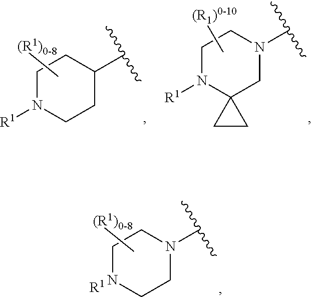

wherein R¹ is as defined herein. In some embodiments, B is selected from,

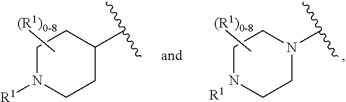

wherein R¹ is as defined herein.

In some embodiments, B is selected from

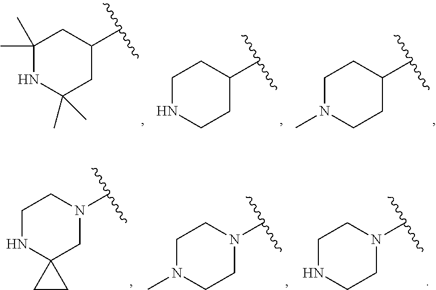

In some embodiments, B is

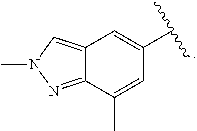 [SMILES: *c1cc(C)c2nn(C)cc2c1]

In some embodiments, B is

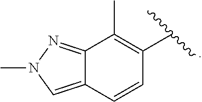 [SMILES: *c1ccc2cn(C)nc2c1C]

In some embodiments, B is

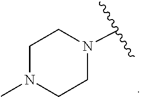 [SMILES: *N1CCN(C)CC1]

In some embodiments, B is

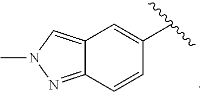 [SMILES: *c1ccc2nn(C)cc2c1]

In some embodiments, B is

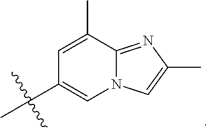 [SMILES: *c1cc(C)c2nc(C)cn2c1]

In some embodiments, B is

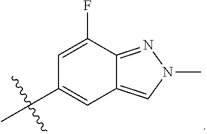 [SMILES: *c1cc(F)c2nn(C)cc2c1]

In some embodiments, B is

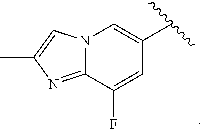 [SMILES: *c1cc(F)c2nc(C)cn2c1]

In some embodiments, B is

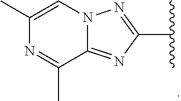 [SMILES: *c1nc2c(C)nc(C)cn2n1]

In some embodiments, B is

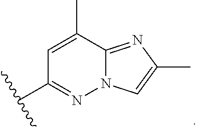 [SMILES: *c1cc(C)c2nc(C)cn2n1]

In some embodiments, B is

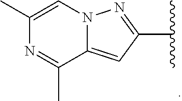 [SMILES: *c1cc2c(C)nc(C)cn2n1]

In some embodiments, B is

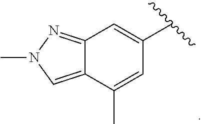 [SMILES: *c1cc(C)c2cn(C)nc2c1]

In some embodiments, B is

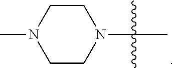 [SMILES: *N1CCN(C)CC1]

In some embodiments, B is

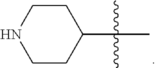 [SMILES: *C1CCNCC1]

In some embodiments, B is

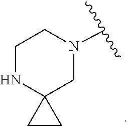 [SMILES: *N1CCNC2(CC2)C1]

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹.

In some embodiments, the compound of Formula (II) is selected from a compound in Table 2, or a pharmaceutically acceptable salt, solvate, hydrate, tautomer, or stereoisomer thereof.

TABLE 2
Exemplary compounds of Formula (II)

| Cmpd | |
|---|---|
| No. | Structure |
| 100 | 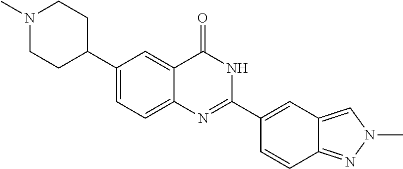 |
| 101 | 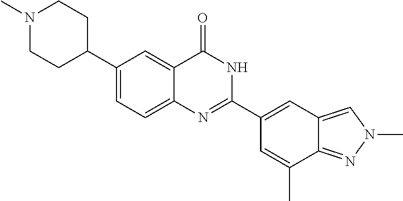 |
| 102 | 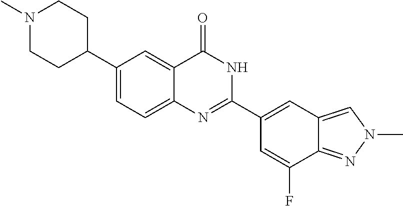 |
| 103 | 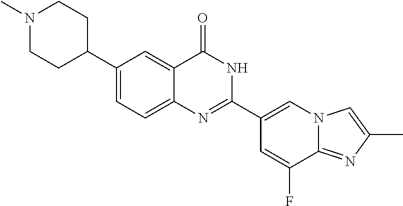 |
| 104 | 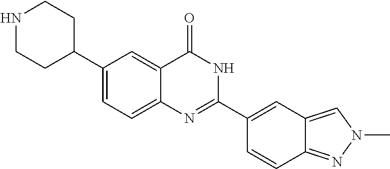 [SMILES: Cn1cc2cc(-c3nc4ccc(C5CCNCC5)cc4c(=O)[nH]3)ccc2n1] |
| 105 | 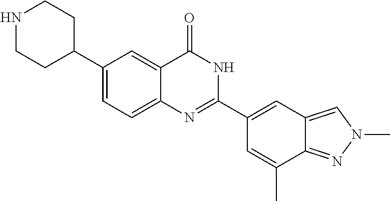 [SMILES: Cc1cc(-c2nc3ccc(C4CCNCC4)cc3c(=O)[nH]2)cc2cn(C)nc12] |
| 106 | 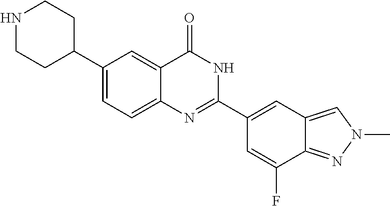 [SMILES: Cn1cc2cc(-c3nc4ccc(C5CCNCC5)cc4c(=O)[nH]3)cc(F)c2n1] |
| 107 | 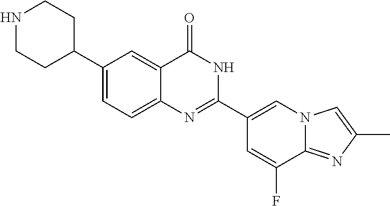 [SMILES: Cc1cn2cc(-c3nc4ccc(C5CCNCC5)cc4c(=O)[nH]3)cc(F)c2n1] |
| 108 | 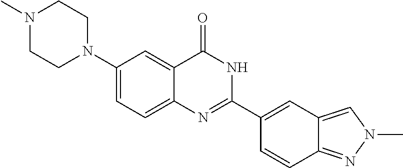 |
| 109 | 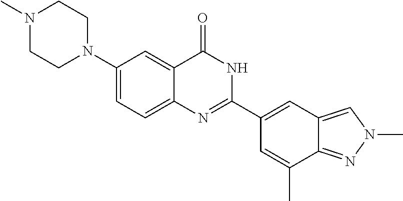 |
| 110 | 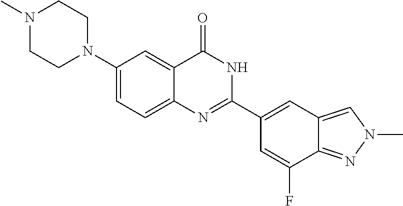 |
| 111 | 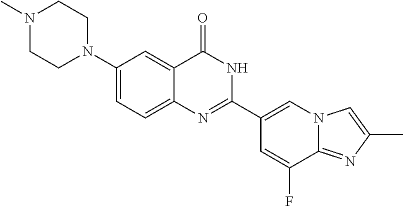 |
| 112 | 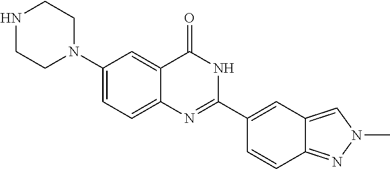 [SMILES: Cn1cc2cc(-c3nc4ccc(N5CCNCC5)cc4c(=O)[nH]3)ccc2n1] |
| 113 | 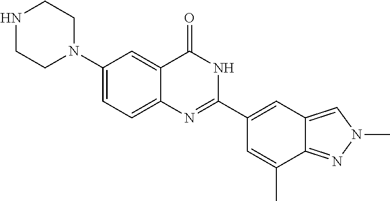 [SMILES: Cc1cc(-c2nc3ccc(N4CCNCC4)cc3c(=O)[nH]2)cc2cn(C)nc12] |
| 114 | 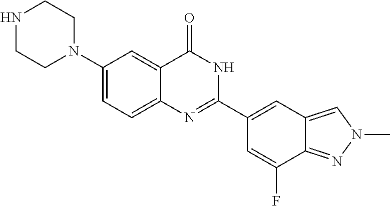 [SMILES: Cn1cc2cc(-c3nc4ccc(N5CCNCC5)cc4c(=O)[nH]3)cc(F)c2n1] |
| 115 | 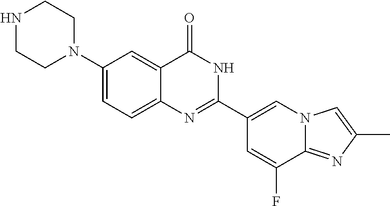 [SMILES: Cc1cn2cc(-c3nc4ccc(N5CCNCC5)cc4c(=O)[nH]3)cc(F)c2n1] |
| 116 | 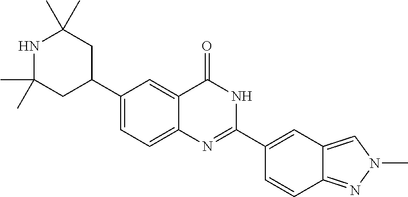 |
| 117 | 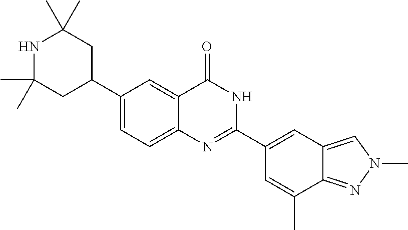 |
| 118 | 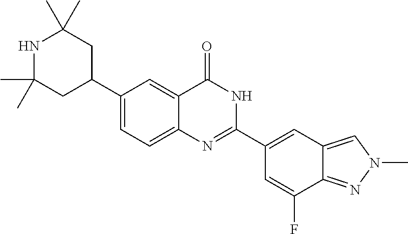 |
| 119 | 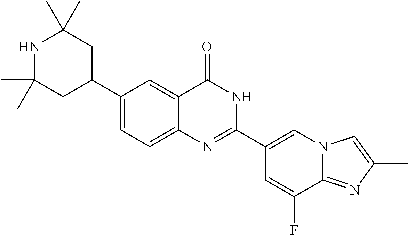 |
| 120 | 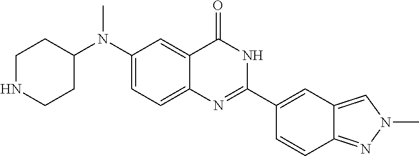 [SMILES: CN(c1ccc2nc(-c3ccc4nn(C)cc4c3)[nH]c(=O)c2c1)C1CCNCC1] |
| 121 | 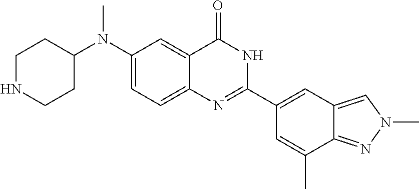 [SMILES: Cc1cc(-c2nc3ccc(N(C)C4CCNCC4)cc3c(=O)[nH]2)cc2cn(C)nc12] |
| 122 | 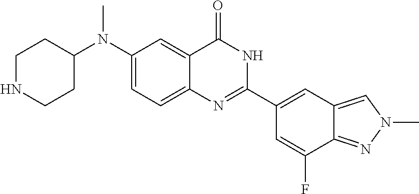 [SMILES: CN(c1ccc2nc(-c3cc(F)c4nn(C)cc4c3)[nH]c(=O)c2c1)C1CCNCC1] |
| 123 | 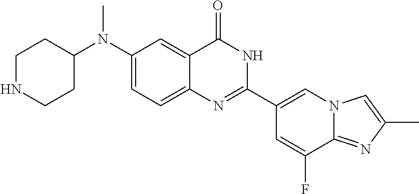 [SMILES: Cc1cn2cc(-c3nc4ccc(N(C)C5CCNCC5)cc4c(=O)[nH]3)cc(F)c2n1] |
| 124 | 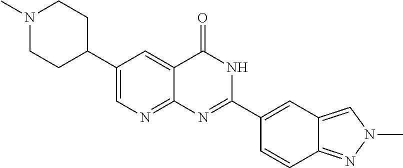 |
| 125 | 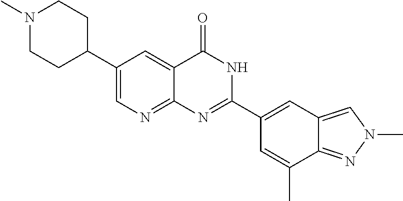 |
| 126 | 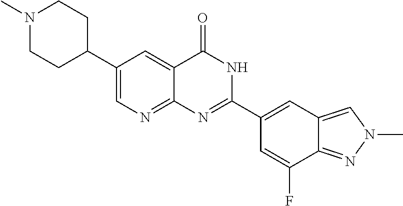 |
| 127 | 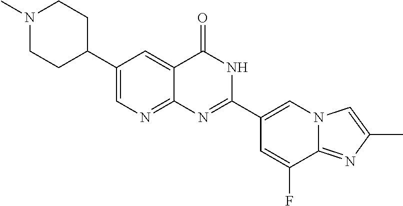 |
| 128 | 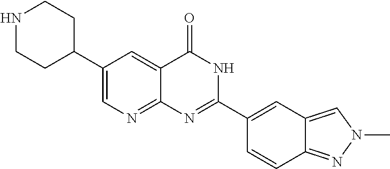 [SMILES: Cn1cc2cc(-c3nc4ncc(C5CCNCC5)cc4c(=O)[nH]3)ccc2n1] |
| 129 | 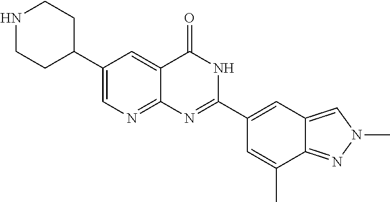 [SMILES: Cc1cc(-c2nc3ncc(C4CCNCC4)cc3c(=O)[nH]2)cc2cn(C)nc12] |
| 130 | 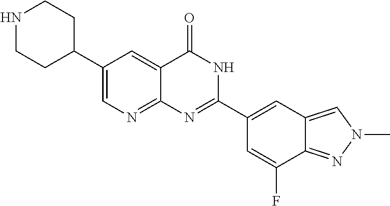 [SMILES: Cn1cc2cc(-c3nc4ncc(C5CCNCC5)cc4c(=O)[nH]3)cc(F)c2n1] |
| 131 | 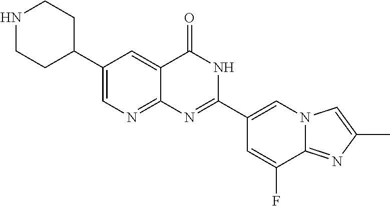 [SMILES: Cc1cn2cc(-c3nc4ncc(C5CCNCC5)cc4c(=O)[nH]3)cc(F)c2n1] |
| 132 | 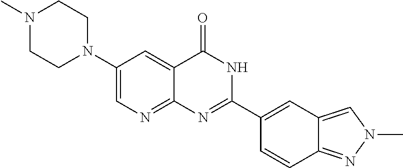 |
| 133 | 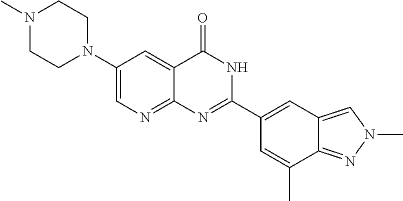 |
| 134 | 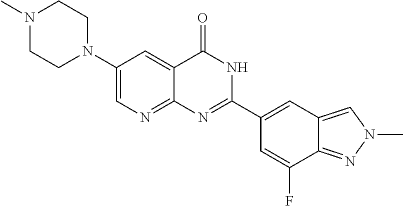 |
| 135 | 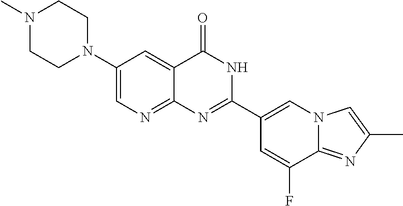 |
| 136 | 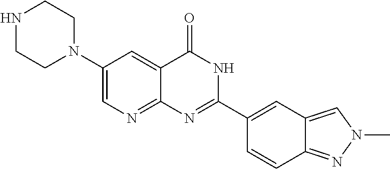 [SMILES: Cn1cc2cc(-c3nc4ncc(N5CCNCC5)cc4c(=O)[nH]3)ccc2n1] |
| 137 | 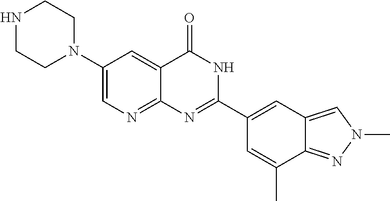 [SMILES: Cc1cc(-c2nc3ncc(N4CCNCC4)cc3c(=O)[nH]2)cc2cn(C)nc12] |
| 138 | 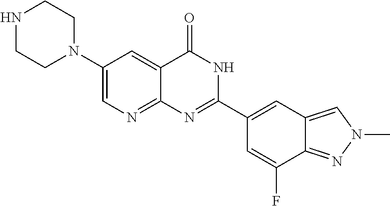 [SMILES: Cn1cc2cc(-c3nc4ncc(N5CCNCC5)cc4c(=O)[nH]3)cc(F)c2n1] |
| 139 | 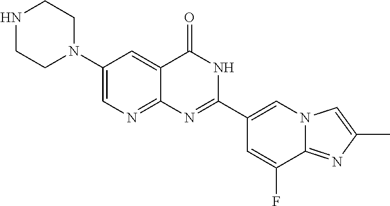 [SMILES: Cc1cn2cc(-c3nc4ncc(N5CCNCC5)cc4c(=O)[nH]3)cc(F)c2n1] |
| 140 | 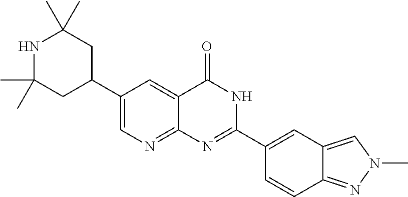 |
| 141 | 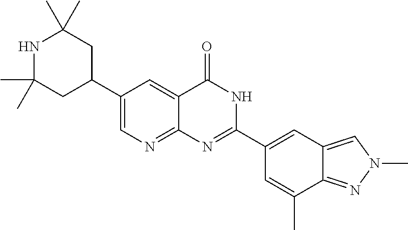 |
| 142 | 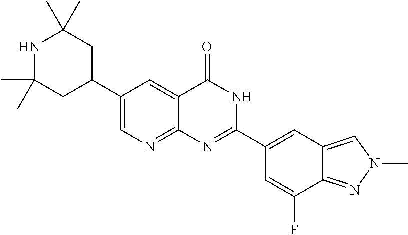 |
| 143 | 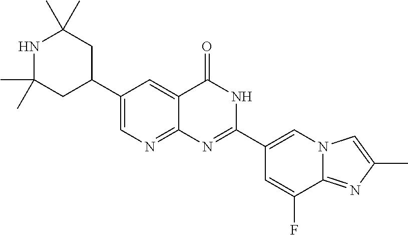 |
| 144 | 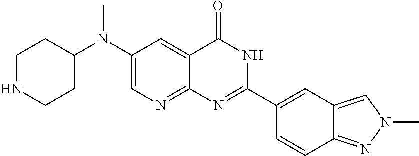 [SMILES: CN(c1cnc2nc(-c3ccc4nn(C)cc4c3)[nH]c(=O)c2c1)C1CCNCC1] |
| 145 | 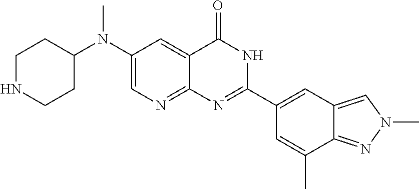 [SMILES: Cc1cc(-c2nc3ncc(N(C)C4CCNCC4)cc3c(=O)[nH]2)cc2cn(C)nc12] |
| 146 | 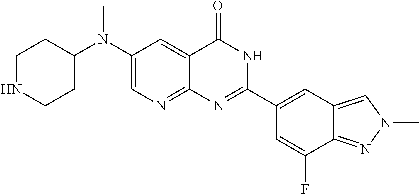 [SMILES: CN(c1cnc2nc(-c3cc(F)c4nn(C)cc4c3)[nH]c(=O)c2c1)C1CCNCC1] |
| 147 | 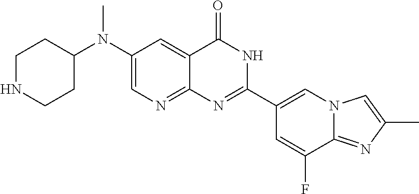 [SMILES: Cc1cn2cc(-c3nc4ncc(N(C)C5CCNCC5)cc4c(=O)[nH]3)cc(F)c2n1] |
| 165 | 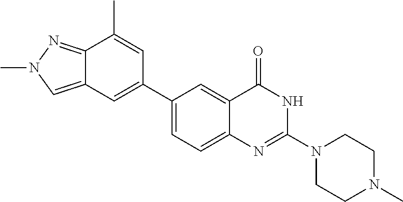 |
| 166 | 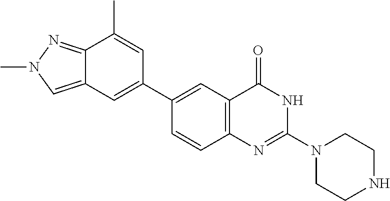 [SMILES: Cc1cc(-c2ccc3nc(N4CCNCC4)[nH]c(=O)c3c2)cc2cn(C)nc12] |
| 167 | 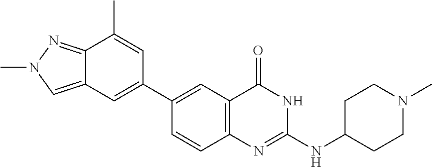 |
| 189 | 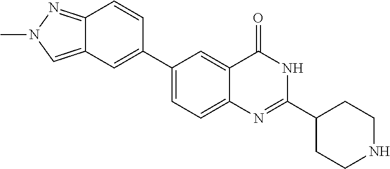 [SMILES: Cn1cc2cc(-c3ccc4nc(C5CCNCC5)[nH]c(=O)c4c3)ccc2n1] |
| 190 | 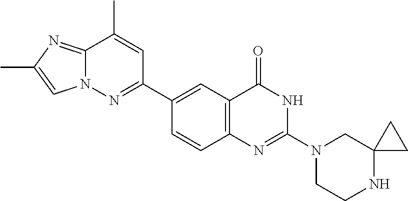 [SMILES: Cc1cn2nc(-c3ccc4nc(N5CCNC6(CC6)C5)[nH]c(=O)c4c3)cc(C)c2n1] |
| 191 | 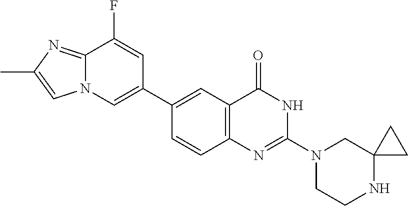 [SMILES: Cc1cn2cc(-c3ccc4nc(N5CCNC6(CC6)C5)[nH]c(=O)c4c3)cc(F)c2n1] |
| 192 | 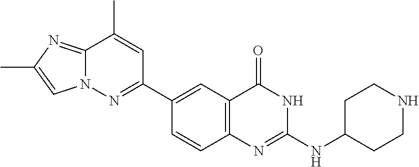 [SMILES: Cc1cn2nc(-c3ccc4nc(NC5CCNCC5)[nH]c(=O)c4c3)cc(C)c2n1] |
| 193 | 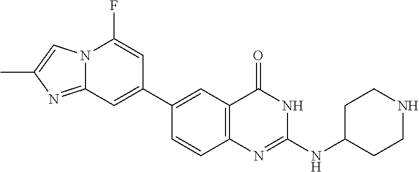 [SMILES: Cc1cn2c(F)cc(-c3ccc4nc(NC5CCNCC5)[nH]c(=O)c4c3)cc2n1] |
| 238 | 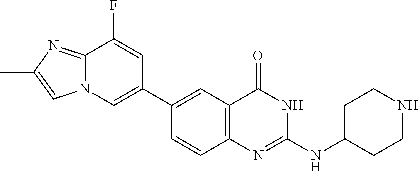 [SMILES: Cc1cn2cc(-c3ccc4nc(NC5CCNCC5)[nH]c(=O)c4c3)cc(F)c2n1] |
| 239 | 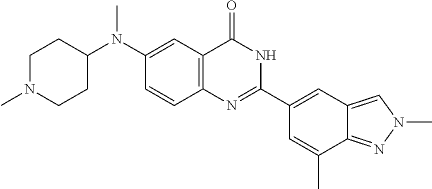 |
| 483 | 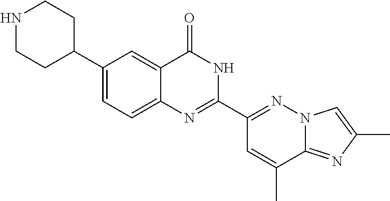 [SMILES: Cc1cn2nc(-c3nc4ccc(C5CCNCC5)cc4c(=O)[nH]3)cc(C)c2n1] |
| 484 | 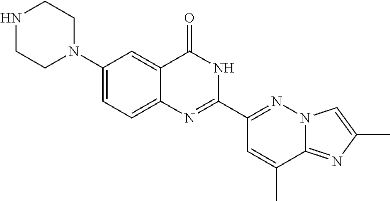 [SMILES: Cc1cn2nc(-c3nc4ccc(N5CCNCC5)cc4c(=O)[nH]3)cc(C)c2n1] |

In some embodiments, for Formula (II), A is monocyclic heterocyclyl (e.g., N-methyl piperidinyl); B is bicyclic heteroaryl (e.g., 2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 100, or a pharmaceutically acceptable salt, solvate, hydrate, tautomer, or stereoisomer thereof.

In some embodiments, for Formula (II), A is monocyclic heterocyclyl (e.g., N-methyl piperidinyl); B is bicyclic heteroaryl (e.g., 2,7-dimethyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 101, or a pharmaceutically acceptable salt, solvate, hydrate, tautomer, or stereoisomer thereof.

In some embodiments, for Formula (II), A is monocyclic heterocyclyl (e.g., N-methyl piperidinyl); B is bicyclic heteroaryl (e.g., 7-fluoro-2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 102, or a pharmaceutically acceptable salt, solvate, hydrate, tautomer, or stereoisomer thereof.

In some embodiments, for Formula (II), A is monocyclic heterocyclyl (e.g., N-methyl piperidinyl); B is bicyclic heteroaryl (e.g., 8-fluoro-2-methylimidazo[1,2-a]pyridinyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 103, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 104, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2,7-dimethyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 105, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 7-fluoro-2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 106, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 8-fluoro-2-methylimidazo[1,2-a]pyridinyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 107, or a pharmaceutically acceptable salt, solvate, hydrate, tautomer, or stereoisomer thereof.

In some embodiments, for Formula (II), A is monocyclic heterocyclyl (e.g., N-methyl piperazinyl); B is bicyclic heteroaryl (e.g., 2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 108, or a pharmaceutically acceptable salt, solvate, hydrate, tautomer, or stereoisomer thereof.

In some embodiments, for Formula (II), A is monocyclic heterocyclyl (e.g., N-methyl piperazinyl); B is bicyclic heteroaryl (e.g., 2,7-dimethyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 109, or a pharmaceutically acceptable salt, solvate, hydrate, tautomer, or stereoisomer thereof.

In some embodiments, for Formula (II), A is monocyclic heterocyclyl (e.g., N-methyl piperazinyl); B is bicyclic heteroaryl (e.g., 7-fluoro-2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 110, or a pharmaceutically acceptable salt, solvate, hydrate, tautomer, or stereoisomer thereof.

In some embodiments, for Formula (II), A is monocyclic heterocyclyl (e.g., N-methyl piperazinyl); B is bicyclic heteroaryl (e.g., 8-fluoro-2-methylimidazo[1,2-a]pyridinyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 111, or a pharmaceutically acceptable salt, solvate, hydrate, tautomer, or stereoisomer thereof.

In some embodiments, for Formula (II), A is monocyclic heterocyclyl (e.g., piperazinyl); B is bicyclic heteroaryl (e.g., 2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 112, or a pharmaceutically acceptable salt, solvate, hydrate, tautomer, or stereoisomer thereof.

In some embodiments, for Formula (II), A is monocyclic heterocyclyl (e.g., piperazinyl); B is bicyclic heteroaryl (e.g., 2,7-dimethyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 113, or a pharmaceutically acceptable salt, solvate, hydrate, tautomer, or stereoisomer thereof.

In some embodiments, for Formula (II), A is monocyclic heterocyclyl (e.g., piperazinyl); B is bicyclic heteroaryl (e.g., 7-fluoro-2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 114, or a pharmaceutically acceptable salt, solvate, hydrate, tautomer, or stereoisomer thereof.

In some embodiments, for Formula (II), A is monocyclic heterocyclyl (e.g., piperazinyl); B is bicyclic heteroaryl (e.g., 8-fluoro-2-methylimidazo[1,2-a]pyridinyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 115, or a pharmaceutically acceptable salt, solvate, hydrate, tautomer, or stereoisomer thereof.

In some embodiments, for Formula (II), A is monocyclic heterocyclyl (e.g., 2,2,6,6-tetramethylpiperidinyl); B is bicyclic heteroaryl (e.g., 2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 116, or a pharmaceutically acceptable salt, solvate, hydrate, tautomer, or stereoisomer thereof.

In some embodiments, for Formula (II), A is monocyclic heterocyclyl (e.g., 2,2,6,6-tetramethylpiperidinyl); B is bicyclic heteroaryl (e.g., 2,7-dimethyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 117, or a pharmaceutically acceptable salt, solvate, hydrate, tautomer, or stereoisomer thereof.

In some embodiments, for Formula (II), A is monocyclic heterocyclyl (e.g., 2,2,6,6-tetramethylpiperidinyl); B is bicyclic heteroaryl (e.g., 7-fluoro-2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 118, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 119, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2-methyl-2H-indazolyl); L¹ is —N(R⁸)— (e.g., —N(CH₃)—); L² is absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 120, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2,7-dimethyl-2H-indazolyl); L¹ is —N(R⁸)— (e.g., —N(CH₃)—); L² is absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 121, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 7-fluoro-2-methyl-2H-indazolyl); L¹ is —N(R⁸)— (e.g., —N(CH₃)—); L² is absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 122, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 8-fluoro-2-methylimidazo[1,2-a]pyridinyl); L¹ is —N(R⁸)— (e.g., —N(CH₃)—); L² is absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 123, or a pharmaceutically acceptable salt, solvate, hydrate, tautomer, or stereoisomer thereof.

In some embodiments, for Formula (II), A is monocyclic heterocyclyl (e.g., N-methyl piperidinyl); B is bicyclic heteroaryl (e.g., 2-methyl-2H-indazolyl); L¹ and L² are each absent; X, and W are each independently C(R³) (e.g., CH); Z is N; Y is N(R^{4a}) (e.g., NH); and R² is absent. In some embodiments, the compound of Formula (II) and (II-a) is Compound 124, or a pharmaceutically acceptable salt, solvate, hydrate, tautomer, or stereoisomer thereof.

In some embodiments, for Formula (II), A is monocyclic heterocyclyl (e.g., N-methyl piperidinyl); B is bicyclic heteroaryl (e.g., 2,7-dimethyl-2H-indazolyl); L¹ and L² are each absent; X and W are each independently C(R³) (e.g., CH); Z in N; Y is N(R^{4a}) (e.g., NH); and R² is absent. In some embodiments, the compound of Formula (II) and (II-a) is Compound 125, or a pharmaceutically acceptable salt, solvate, hydrate, tautomer, or stereoisomer thereof.

In some embodiments, for Formula (II), A is monocyclic heterocyclyl (e.g., N-methyl piperidinyl); B is bicyclic heteroaryl (e.g., 7-fluoro-2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 126, or a pharmaceutically acceptable salt, solvate, hydrate, tautomer, or stereoisomer thereof.

In some embodiments, for Formula (II), A is monocyclic heterocyclyl (e.g., N-methyl piperidinyl); B is bicyclic heteroaryl (e.g., 8-fluoro-2-methylimidazo[1,2-a]pyridinyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 127, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2-methyl-2H-indazolyl); L¹ and L² are each absent; X, and W are each independently C(R³) (e.g., CH); Z is N; Y is N(R^{4a}) (e.g., NH); and R² is absent. In some embodiments, the compound of Formula (II) and (II-a) is Compound 128, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2,7-dimethyl-2H-indazolyl); L¹ and L² are each absent; X and W are each independently C(R³) (e.g., CH); Z in N; Y is N(R^{4a}) (e.g., NH); and R² is absent. In some embodiments, the compound of Formula (II) and (II-a) is Compound 129, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 7-fluoro-2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 130, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 8-fluoro-2-methylimidazo[1,2-a]pyridinyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 131, or a pharmaceutically acceptable salt, solvate, hydrate, tautomer, or stereoisomer thereof.

In some embodiments, for Formula (II), A is monocyclic heterocyclyl (e.g., N-methyl piperazyl); B is bicyclic heteroaryl (e.g., 2-methyl-2H-indazolyl); L¹ and L² are each absent; X, and W are each independently C(R³) (e.g., CH); Z is N; Y is N(R^{4a}) (e.g., NH); and R² is absent. In some embodiments, the compound of Formula (II) and (II-a) is Compound 132, or a pharmaceutically acceptable salt, solvate, hydrate, tautomer, or stereoisomer thereof.

In some embodiments, for Formula (II), A is monocyclic heterocyclyl (e.g., N-methyl piperazyl); B is bicyclic heteroaryl (e.g., 2,7-dimethyl-2H-indazolyl); L¹ and L² are each absent; X and W are each independently C(R³) (e.g., CH); Z in N; Y is N(R^{4a}) (e.g., NH); and R² is absent. In some embodiments, the compound of Formula (II) and (II-a) is Compound 133, or a pharmaceutically acceptable salt, solvate, hydrate, tautomer, or stereoisomer thereof.

In some embodiments, for Formula (II), A is monocyclic heterocyclyl (e.g., N-methyl piperazyl); B is bicyclic heteroaryl (e.g., 7-fluoro-2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 134, or a pharmaceutically acceptable salt, solvate, hydrate, tautomer, or stereoisomer thereof.

In some embodiments, for Formula (II), A is monocyclic heterocyclyl (e.g., N-methyl piperazyl); B is bicyclic heteroaryl (e.g., 8-fluoro-2-methylimidazo[1,2-a]pyridinyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 135, or a pharmaceutically acceptable salt, solvate, hydrate, tautomer, or stereoisomer thereof.

In some embodiments, for Formula (II), A is monocyclic heterocyclyl (e.g., piperazyl); B is bicyclic heteroaryl (e.g., 2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 136, or a pharmaceutically acceptable salt, solvate, hydrate, tautomer, or stereoisomer thereof.

In some embodiments, for Formula (II), A is monocyclic heterocyclyl (e.g., piperazyl); B is bicyclic heteroaryl (e.g., 2,7-dimethyl-2H-indazolyl); L¹ and L² are each absent; X and W are each independently C(R³) (e.g., CH); Z in N; Y is N(R^{4a}) (e.g., NH); and R² is absent. In some embodiments, the compound of Formula (II) and (II-a) is Compound 137, or a pharmaceutically acceptable salt, solvate, hydrate, tautomer, or stereoisomer thereof.

In some embodiments, for Formula (II), A is monocyclic heterocyclyl (e.g., piperazyl); B is bicyclic heteroaryl (e.g., 7-fluoro-2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 138, or a pharmaceutically acceptable salt, solvate, hydrate, tautomer, or stereoisomer thereof.

In some embodiments, for Formula (II), A is monocyclic heterocyclyl (e.g., piperazyl); B is bicyclic heteroaryl (e.g., 8-fluoro-2-methylimidazo[1,2-a]pyridinyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 139, or a pharmaceutically acceptable salt, solvate, hydrate, tautomer, or stereoisomer thereof.

In some embodiments, for Formula (II), A is monocyclic heterocyclyl (e.g., 2,2,6,6-tetramethylpiperidinyl); B is bicyclic heteroaryl (e.g., 2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 140, or a pharmaceutically acceptable salt, solvate, hydrate, tautomer, or stereoisomer thereof.

In some embodiments, for Formula (II), A is monocyclic heterocyclyl (e.g., 2,2,6,6-tetramethylpiperidinyl); B is bicyclic heteroaryl (e.g., 2,7-dimethyl-2H-indazolyl); L¹ and L² are each absent; X and W are each independently C(R³) (e.g., CH); Z in N; Y is N(R^{4a}) (e.g., NH); and R² is absent. In some embodiments, the compound of Formula (II) and (II-a) is Compound 141, or a pharmaceutically acceptable salt, solvate, hydrate, tautomer, or stereoisomer thereof.

In some embodiments, for Formula (II), A is monocyclic heterocyclyl (e.g., 2,2,6,6-tetramethylpiperidinyl); B is bicyclic heteroaryl (e.g., 7-fluoro-2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 142, or a pharmaceutically acceptable salt, solvate, hydrate, tautomer, or stereoisomer thereof.

In some embodiments, for Formula (II), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L¹ and L² are each absent; X and W are each independently C(R³) (e.g., CH); Z is N; Y is N(R^{4a}) (e.g., NH); and R² is absent. In some embodiments, the compound of Formula (II) and (II-a) is Compound 143, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2-methyl-2H-indazolyl); L¹ is —N(R⁸)— (e.g., —N(CH₃)—); L² is absent; X and W are each independently C(R³) (e.g., CH); Z is N; Y is N(R^{4a}) (e.g., NH); and R² is absent. In some embodiments, the compound of Formula (II) and (II-a) is Compound 144, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2,7-dimethyl-2H-indazolyl); L¹ is —N(R⁸)— (e.g., —N(CH₃)—); L² is absent; X and W are each independently C(R³) (e.g., CH); Z in N; Y is N(R^{4a}) (e.g., NH); and R² is absent. In some embodiments, the compound of Formula (II) and (II-a) is Compound 145, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 7-fluoro-2-methyl-2H-indazolyl); L¹ is —N(R⁸)— (e.g., —N(CH₃)—); L² is absent; X and W are each independently C(R³) (e.g., CH); Z is N; Y is N(R^{4a}) (e.g., NH); and R² is absent. In some embodiments, the compound of Formula (II) and (II-a) is Compound 146, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 8-fluoro-2-methylimidazo[1,2-a]pyridinyl); L¹ is —N(R⁸)— (e.g., —N(CH₃)—); L² is absent; X and W are each independently C(R³) (e.g., CH); Z is N; Y is N(R^{4a}) (e.g., NH); and R² is absent. In some embodiments, the compound of Formula (II) and (II-a) is Compound 147, or a pharmaceutically acceptable salt, solvate, hydrate, tautomer, or stereoisomer thereof.

In some embodiments, for Formula (II), A is bicyclic heterocyclyl (e.g., 2,7-dimethyl-2H-indazolyl); B is monocyclic heteroaryl (e.g., N-methyl piperazyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 165, or a pharmaceutically acceptable salt, solvate, hydrate, tautomer, or stereoisomer thereof.

In some embodiments, for Formula (II), A is bicyclic heterocyclyl (e.g., 2,7-dimethyl-2H-indazolyl); B is monocyclic heteroaryl (e.g., piperazyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 166, or a pharmaceutically acceptable salt, solvate, hydrate, tautomer, or stereoisomer thereof.

In some embodiments, for Formula (II), A is bicyclic heterocyclyl (e.g., 2,7-dimethyl-2H-indazolyl); B is monocyclic heteroaryl (e.g., N-methyl piperidinyl); L¹ is absent; L² is —N(R⁸)—(e.g., —N(H)—); X, W, and Z are each independently C(R³) (e.g., CH); Y is N(R^{4a}) (e.g., NH); and R² is absent. In some embodiments, the compound of Formula (II), (II-a), (II-b), and (II-c) is Compound 167, or a pharmaceutically acceptable salt, solvate, hydrate, tautomer, or stereoisomer thereof.

In some embodiments, for Formula (II), A is bicyclic heterocyclyl (e.g., 2-methyl-2H-indazolyl); B is monocyclic heteroaryl (e.g., piperidinyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 189, or a pharmaceutically acceptable salt, solvate, hydrate, tautomer, or stereoisomer thereof.

In some embodiments, for Formula (II), A is bicyclic heterocyclyl (e.g., 2,8-dimethylimidazo[1,2-b]pyridazyl); B is monocyclic heteroaryl (e.g., 4,7-diazaspiro[2.5]octanyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 190, or a pharmaceutically acceptable salt, solvate, hydrate, tautomer, or stereoisomer thereof.

In some embodiments, for Formula (II), A is bicyclic heterocyclyl (e.g., 8-fluoro-2-methylimidazo[1,2-a]pyridinyl); B is monocyclic heteroaryl (e.g., 4,7-diazaspiro[2.5]octanyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 191, or a pharmaceutically acceptable salt, solvate, hydrate, tautomer, or stereoisomer thereof.

In some embodiments, for Formula (II), A is bicyclic heterocyclyl (e.g., 2,8-dimethylimidazo[1,2-b]pyridazyl); B is monocyclic heteroaryl (e.g., piperidinyl); L¹ is absent; L² is —N(R⁸)—(e.g., —N(H)—); X, W, and Z are each independently C(R³) (e.g., CH); Y is N(R^{4a}) (e.g., NH); and R² is absent. In some embodiments, the compound of Formula (II) is Compound 192, or a pharmaceutically acceptable salt, solvate, hydrate, tautomer, or stereoisomer thereof.

In some embodiments, for Formula (II), A is bicyclic heterocyclyl (e.g., 5-fluoro-2-methylimidazo[1,2-a]pyridinyl); B is monocyclic heteroaryl (e.g., piperidinyl); L¹ is absent; L² is —N(R⁸)—(e.g., —N(H)—); X, W, and Z are each independently C(R³) (e.g., CH); Y is N(R^{4a}) (e.g., NH); and R² is absent. In some embodiments, the compound of Formula (II) is Compound 193, or a pharmaceutically acceptable salt, solvate, hydrate, tautomer, or stereoisomer thereof.

In some embodiments, for Formula (II), A is bicyclic heterocyclyl (e.g., 8-fluoro-2-methylimidazo[1,2-a]pyridinyl); B is monocyclic heteroaryl (e.g., piperidinyl); L¹ is absent; L² is —N(R⁸)—(e.g., —N(H)—); X, W, and Z are each independently C(R³) (e.g., CH); Y is N(R^{4a}) (e.g., NH); and R² is absent. In some embodiments, the compound of Formula (II) is Compound 238, or a pharmaceutically acceptable salt, solvate, hydrate, tautomer, or stereoisomer thereof.

In some embodiments, for Formula (II), A is monocyclic heterocyclyl (e.g., N-methyl piperidinyl); B is bicyclic heteroaryl (e.g., 2,7-dimethyl-2H-indazolyl); L¹ is —N(R⁸)—(e.g., —N(CH₃)—); L² is absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 239, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2,8-dimethylimidazo[1,2-b]pyridazinyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 483, or a pharmaceutically acceptable salt, solvate, hydrate, tautomer, or stereoisomer thereof.

In some embodiments, for Formula (II), A is monocyclic heterocyclyl (e.g., piperazinyl); B is bicyclic heteroaryl (e.g., 2,8-dimethylimidazo[1,2-b]pyridazinyl); L¹ and L² are each absent; X, W, and Z are each independently C(R³) (e.g., CH); Y is N(R^{4a}) (e.g., NH); and R² is absent. In some embodiments, the compound of Formula (II), (II-a), (II-b), and (II-c) is Compound 484, or a pharmaceutically acceptable salt, solvate, hydrate, tautomer, or stereoisomer thereof.

As generally described for Formula (III), Y may be N, C, or C(R^{4b}), wherein the dashed lines in the ring comprising Y may be single or double bonds as valency permits. In some embodiments, Y is N or C. In some embodiments, Y is N (e.g., N). In some embodiments, Y is C.

In some embodiments, Z is C(R³) and Y is N. In some embodiments, Z is CH and Y is N. In some embodiments, X is C(R³) and Y is N. In some embodiments, X is CH and Y is N. In some embodiments, Z is C(R³) and Y is N. In some embodiments, Z is CH and Y is N. In some embodiments, Z and X are independently C(R³) and Y is N. In some embodiments, Z and X are independently CH and Y is N. In some embodiments, X and Z are independently C(R³) and Y is N. In some embodiments, X and Z are independently C(R³) and Y is N. In some embodiments, X and Z are independently CH and Y is N.

In some embodiments, the compound of Formula (III) is a compound of Formula (III-a):

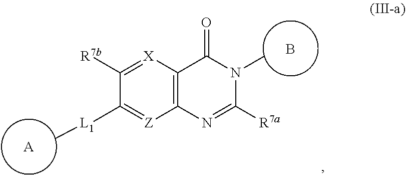

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of X and Z is independently C(R³) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R^{7a} is hydrogen, C₁-C₆-alkyl, C₁-C₆-heteroalkyl, C₁-C₆-haloalkyl, halo, cyano, oxo, or —OR^{A}; R^{7b} is hydrogen, C₁-C₆-alkyl, C₁-C₆-heteroalkyl, C₁-C₆-haloalkyl, halo, cyano, or —OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is

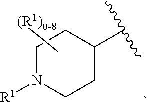

wherein each R¹ is independently hydrogen or C₁-C₆-alkyl. In some embodiments, A is

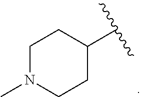 [SMILES: *C1CCN(C)CC1]

In some embodiments, A is

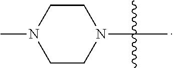 [SMILES: *N1CCN(C)CC1]

In some embodiments, A is

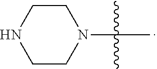 [SMILES: *N1CCNCC1]

In some embodiments, A is heteroaryl optionally substituted with one or more R¹. In some embodiments, A is bicyclic nitrogen-containing heteroaryl. In some embodiments, A is optionally substituted indazolyl. In some embodiments, A is optionally substituted imidazo[1,2-a]pyridinyl. In some embodiments, A is

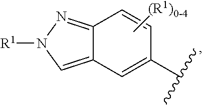

wherein each R¹ is as defined herein. In some embodiments, A is

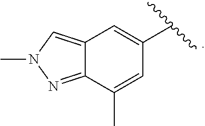 [SMILES: *c1cc(C)c2nn(C)cc2c1]

In some embodiments, A is

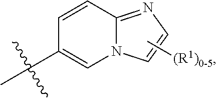

wherein each R⁸ is as defined herein. In some embodiments, A is

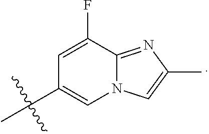 [SMILES: *c1cc(F)c2nc(C)cn2c1]

In some embodiments, A is

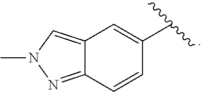 [SMILES: *c1ccc2nn(C)cc2c1]

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

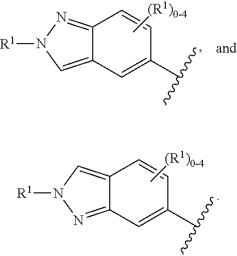

In some embodiments, B is selected from

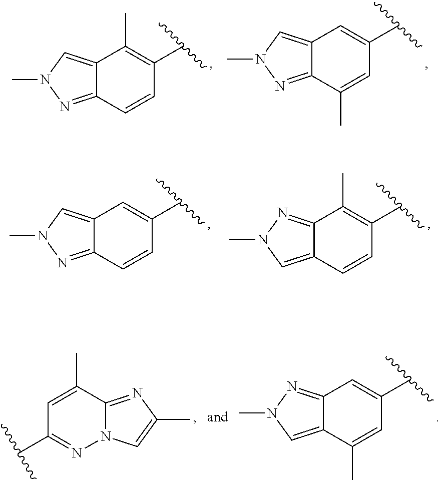

In some embodiments, B is

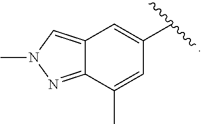 [SMILES: *c1cc(C)c2nn(C)cc2c1]

In some embodiments, B is

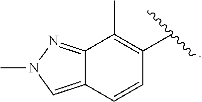 [SMILES: *c1ccc2cn(C)nc2c1C]

In some embodiments, B is heterocyclyl optionally substituted with one or more R¹. In some embodiments, B is monocyclic nitrogen-containing heterocyclyl. In some embodiments, B is optionally substituted piperazinyl. In some embodiments, B is

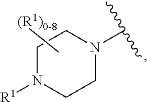

wherein R¹ is as defined herein. In some embodiments, B is

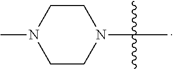 [SMILES: *N1CCN(C)CC1]

In some embodiments, B is

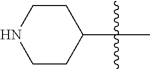 [SMILES: *C1CCNCC1]

In some embodiments, L¹ is absent.

In some embodiments, each of X and Z may independently be N or C(R³). In some embodiments, X is C(R³) (e.g., CH). In some embodiments, X is N. In some embodiments, Z is C(R³) (e.g., CH). In some embodiments, Z is N. In some embodiments, each of X and Z is independently C(R³) (e.g., CH). In some embodiments, each of X and Z is independently C(R³) (e.g., CH).

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹. In some embodiments, each of R^{7a} and R^{7b} is independently hydrogen.

In some embodiments, the compound of Formula (III) is a compound of Formula (III-b):

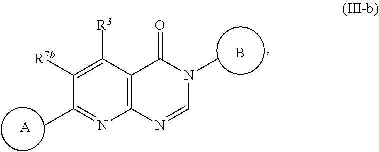

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}, —S(O)ₓR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R^{7b} is hydrogen, C₁-C₆-alkyl, C₁-C₆-heteroalkyl, C₁-C₆-haloalkyl, halo, cyano, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, the compound of Formula (III) is a compound of Formula (III-c):

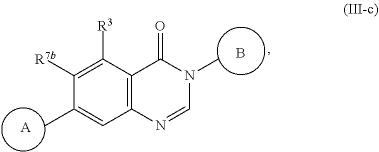

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}, —S(O)ₓR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R^{7b} is hydrogen, C₁-C₆-alkyl, C₁-C₆-heteroalkyl, C₁-C₆-haloalkyl, halo, cyano, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is

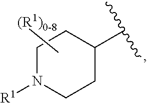

wherein each R¹ is independently hydrogen or C₁-C₆-alkyl. In some embodiments, A is

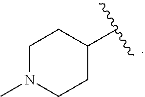 [SMILES: *C1CCN(C)CC1]

In some embodiments, A is

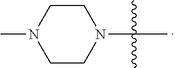 [SMILES: *N1CCN(C)CC1]

In some embodiments, A is

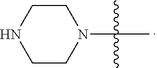 [SMILES: *N1CCNCC1]

In some embodiments, A is heteroaryl optionally substituted with one or more R¹. In some embodiments, A is bicyclic nitrogen-containing heteroaryl. In some embodiments, A is optionally substituted indazolyl. In some embodiments, A is optionally substituted imidazo[1,2-a]pyridinyl. In some embodiments, A is

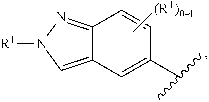

wherein each R¹ is as defined herein. In some embodiments, A is

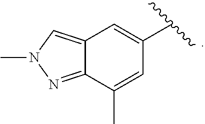 [SMILES: *c1cc(C)c2nn(C)cc2c1]

In some embodiments, A is

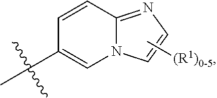

wherein each R¹ is as defined herein. In some embodiments, A is

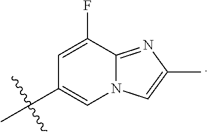 [SMILES: *c1cc(F)c2nc(C)cn2c1]

In some embodiments, A is

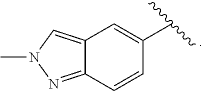 [SMILES: *c1ccc2nn(C)cc2c1]

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

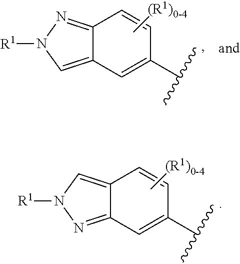

In some embodiments, B is selected from

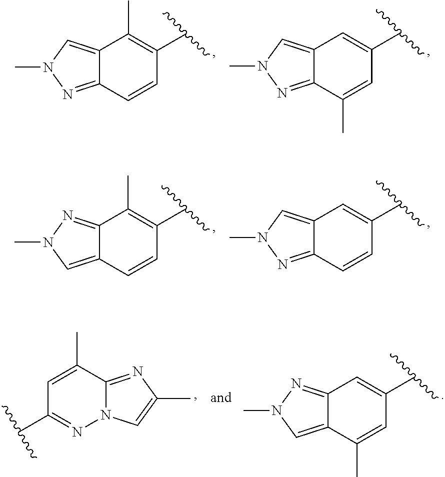 [SMILES: *c1ccc2nn(C)cc2c1C ; *c1cc(C)c2nn(C)cc2c1 ; *c1ccc2cn(C)nc2c1C ; *c1ccc2nn(C)cc2c1 ; *c1cc(C)c2nc(C)cn2n1 ; *c1cc(C)c2cn(C)nc2c1]

In some embodiments, B is

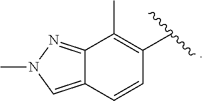 [SMILES: *c1ccc2cn(C)nc2c1C]

In some embodiments, B is

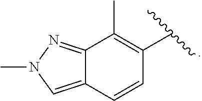 [SMILES: *c1ccc2cn(C)nc2c1C]

In some embodiments, B is

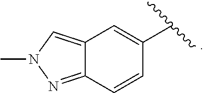 [SMILES: *c1ccc2nn(C)cc2c1]

In some embodiments, Y is N, wherein the dashed lines in the ring comprising Y may be single or double bonds as valency permits. In some embodiments, Y is N or C(R^{4b}). In some embodiments, Y is N (e.g., N). In some embodiments, Y is C(R^{4b}) (e.g., CH).

In some embodiments, L¹ is absent. In some embodiments, R² is absent.

In some embodiments, each of R^{7a} and R^{7b} is independently hydrogen.

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹.

In some embodiments, the compound of Formula (III) is selected from a compound in Table 3, or a pharmaceutically acceptable salt, solvate, hydrate, tautomer, or stereoisomer thereof.

TABLE 3
Exemplary compounds of Formula (III)

| Cmpd | |
|---|---|
| No. | Structure |
| 152 | 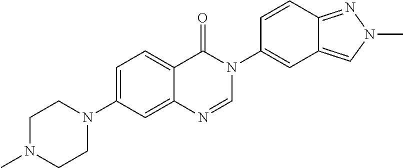 |
| 153 | 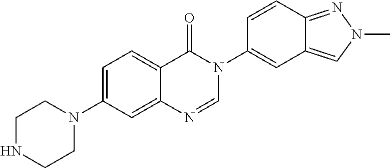 [SMILES: Cn1cc2cc(-n3cnc4cc(N5CCNCC5)ccc4c3=O)ccc2n1] |
| 156 | 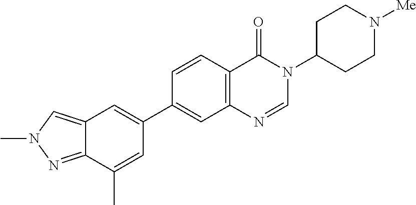 [SMILES: Cc1cc(-c2ccc3c(=O)n(C4CCN(C)CC4)cnc3c2)cc2cn(C)nc12] |
| 157 | 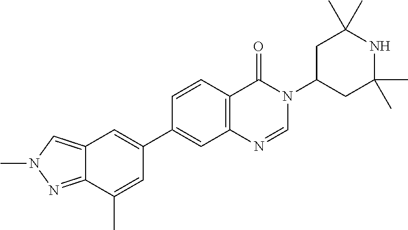 |
| 158 | 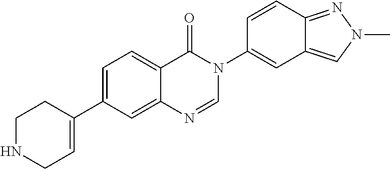 [SMILES: Cn1cc2cc(-n3cnc4cc(C5=CCNCC5)ccc4c3=O)ccc2n1] |
| 159 | 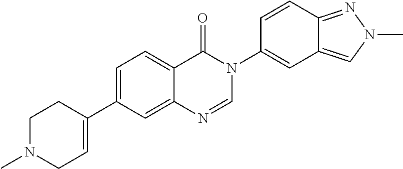 |
| 160 | 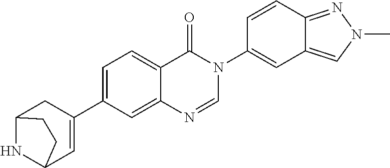 [SMILES: Cn1cc2cc(-n3cnc4cc(C5=CC6CCC(C5)N6)ccc4c3=O)ccc2n1] |
| 161 | 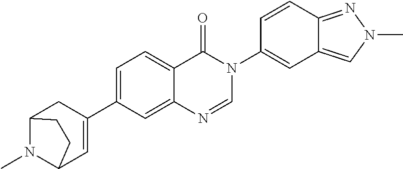 |
| 162 | 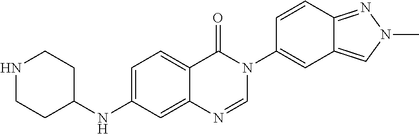 [SMILES: Cn1cc2cc(-n3cnc4cc(NC5CCNCC5)ccc4c3=O)ccc2n1] |
| 163 | 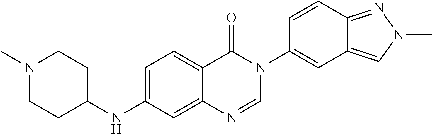 |
| 172 | 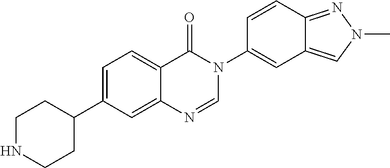 [SMILES: Cn1cc2cc(-n3cnc4cc(C5CCNCC5)ccc4c3=O)ccc2n1] |
| 173 | 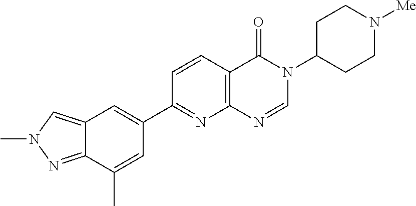 [SMILES: Cc1cc(-c2ccc3c(=O)n(C4CCN(C)CC4)cnc3n2)cc2cn(C)nc12] |
| 174 | 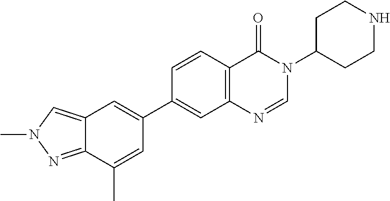 [SMILES: Cc1cc(-c2ccc3c(=O)n(C4CCNCC4)cnc3c2)cc2cn(C)nc12] |
| 175 | 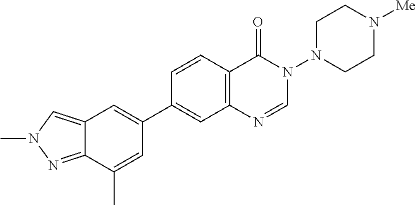 [SMILES: Cc1cc(-c2ccc3c(=O)n(N4CCN(C)CC4)cnc3c2)cc2cn(C)nc12] |
| 176 | 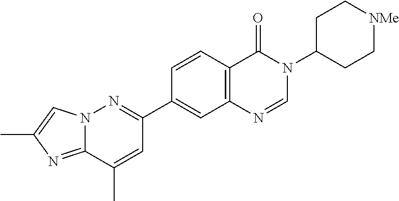 [SMILES: Cc1cn2nc(-c3ccc4c(=O)n(C5CCN(C)CC5)cnc4c3)cc(C)c2n1] |
| 177 | 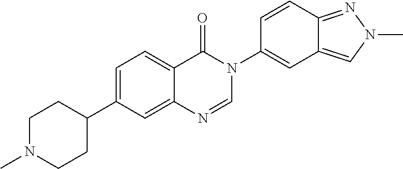 |
| 178 | 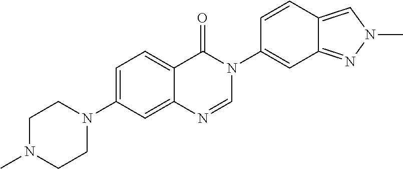 |
| 179 | 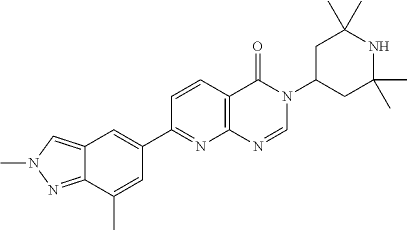 |
| 180 | 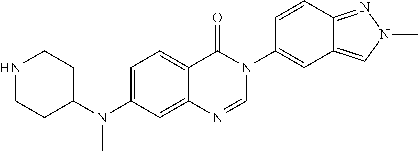 [SMILES: CN(c1ccc2c(=O)n(-c3ccc4nn(C)cc4c3)cnc2c1)C1CCNCC1] |
| 181 | 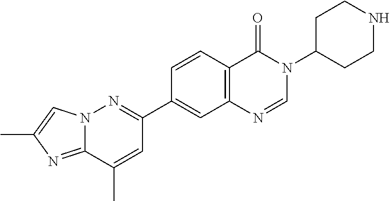 [SMILES: Cc1cn2nc(-c3ccc4c(=O)n(C5CCNCC5)cnc4c3)cc(C)c2n1] |
| 182 | 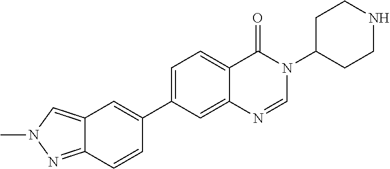 [SMILES: Cn1cc2cc(-c3ccc4c(=O)n(C5CCNCC5)cnc4c3)ccc2n1] |
| 203 | 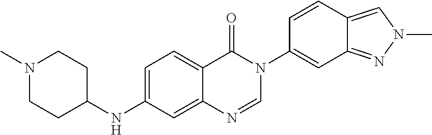 |
| 204 | 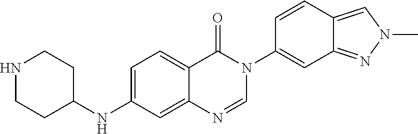 [SMILES: Cn1cc2ccc(-n3cnc4cc(NC5CCNCC5)ccc4c3=O)cc2n1] |
| 205 | 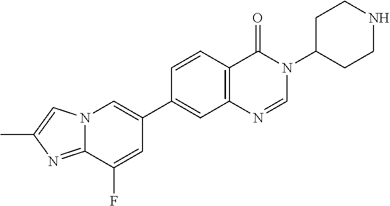 [SMILES: Cc1cn2cc(-c3ccc4c(=O)n(C5CCNCC5)cnc4c3)cc(F)c2n1] |
| 206 | 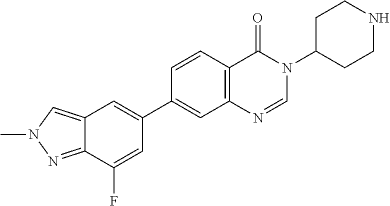 [SMILES: Cn1cc2cc(-c3ccc4c(=O)n(C5CCNCC5)cnc4c3)cc(F)c2n1] |
| 207 | 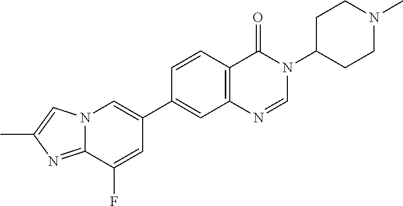 |
| 208 | 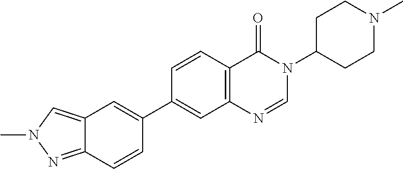 |
| 209 | 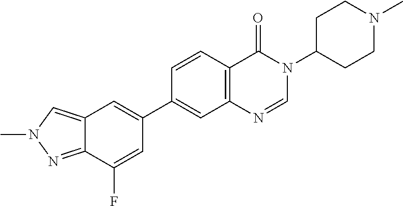 |
| 210 | 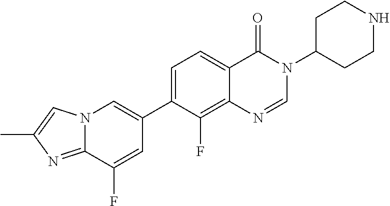 [SMILES: Cc1cn2cc(-c3ccc4c(=O)n(C5CCNCC5)cnc4c3F)cc(F)c2n1] |
| 227 | 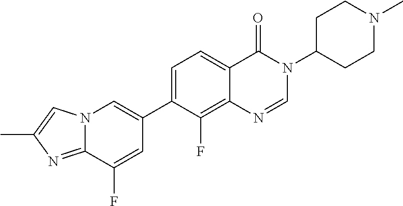 |
| 228 | 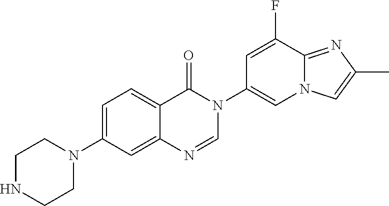 [SMILES: Cc1cn2cc(-n3cnc4cc(N5CCNCC5)ccc4c3=O)cc(F)c2n1] |
| 229 | 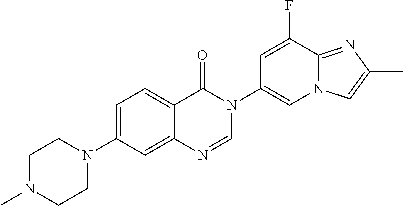 |
| 230 | 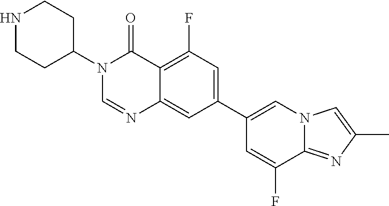 [SMILES: Cc1cn2cc(-c3cc(F)c4c(=O)n(C5CCNCC5)cnc4c3)cc(F)c2n1] |
| 231 | 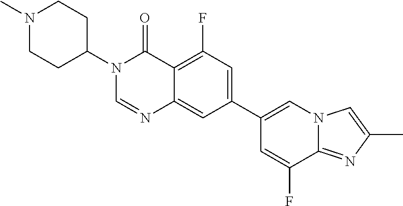 |
| 232 | 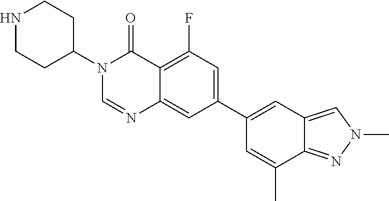 [SMILES: Cc1cc(-c2cc(F)c3c(=O)n(C4CCNCC4)cnc3c2)cc2cn(C)nc12] |
| 233 | 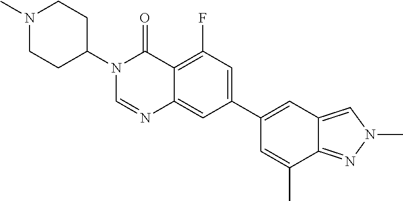 |
| 234 | 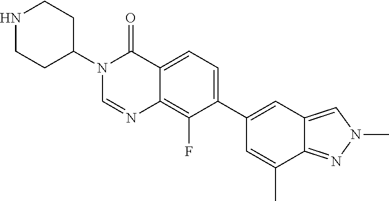 [SMILES: Cc1cc(-c2ccc3c(=O)n(C4CCNCC4)cnc3c2F)cc2cn(C)nc12] |
| 235 | 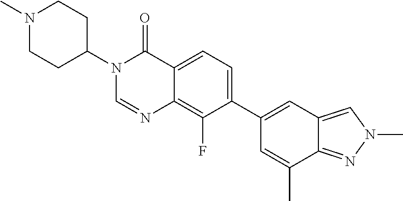 |
| 236 | 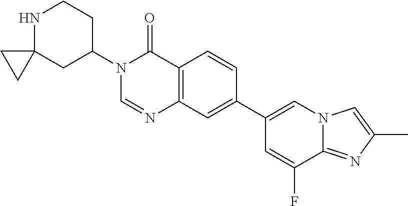 [SMILES: Cc1cn2cc(-c3ccc4c(=O)n(C5CCNC6(CC6)C5)cnc4c3)cc(F)c2n1] |
| 237 | 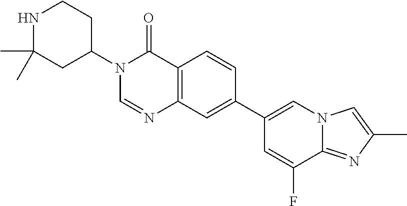 |
| 240 | 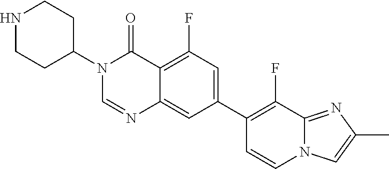 [SMILES: Cc1cn2ccc(-c3cc(F)c4c(=O)n(C5CCNCC5)cnc4c3)c(F)c2n1] |
| 241 | 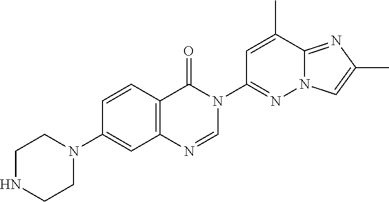 [SMILES: Cc1cn2nc(-n3cnc4cc(N5CCNCC5)ccc4c3=O)cc(C)c2n1] |
| 242 | 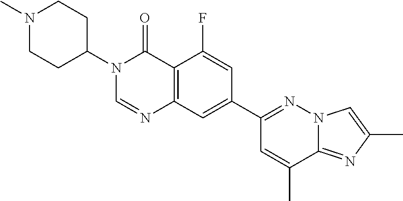 |
| 243 | 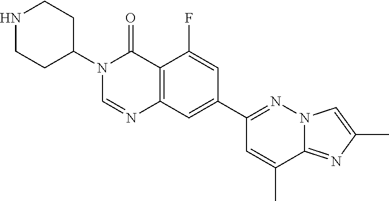 [SMILES: Cc1cn2nc(-c3cc(F)c4c(=O)n(C5CCNCC5)cnc4c3)cc(C)c2n1] |
| 244 | 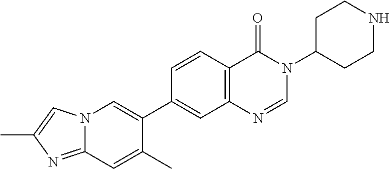 [SMILES: Cc1cn2cc(-c3ccc4c(=O)n(C5CCNCC5)cnc4c3)c(C)cc2n1] |
| 245 | 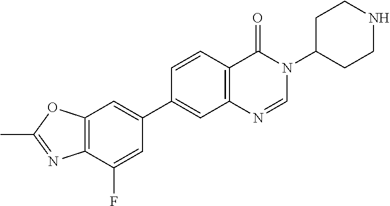 [SMILES: Cc1nc2c(F)cc(-c3ccc4c(=O)n(C5CCNCC5)cnc4c3)cc2o1] |
| 246 | 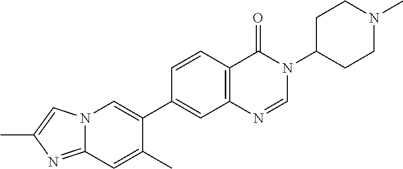 |
| 284 | 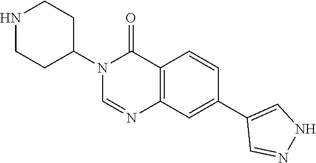 [SMILES: O=c1c2ccc(-c3cn[nH]c3)cc2ncn1C1CCNCC1] |
| 285 | 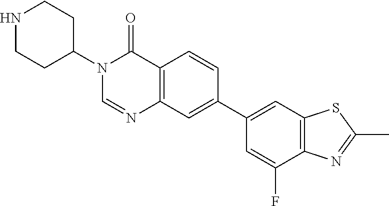 [SMILES: Cc1nc2c(F)cc(-c3ccc4c(=O)n(C5CCNCC5)cnc4c3)cc2s1] |
| 286 | 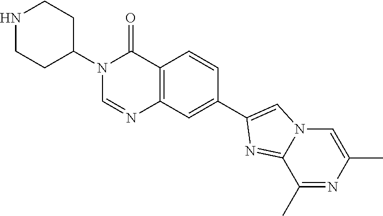 [SMILES: Cc1cn2cc(-c3ccc4c(=O)n(C5CCNCC5)cnc4c3)nc2c(C)n1] |
| 287 | 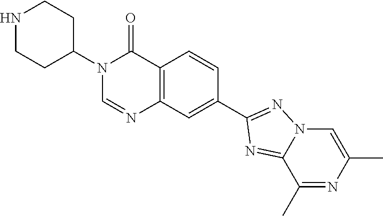 [SMILES: Cc1cn2nc(-c3ccc4c(=O)n(C5CCNCC5)cnc4c3)nc2c(C)n1] |
| 288 | 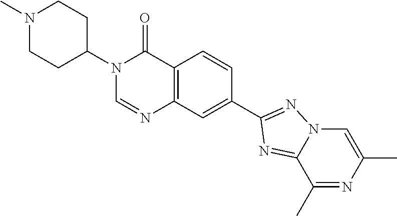 |
| 289 | 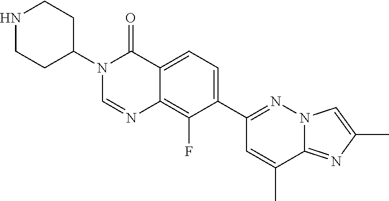 [SMILES: Cc1cn2nc(-c3ccc4c(=O)n(C5CCNCC5)cnc4c3F)cc(C)c2n1] |
| 290 | 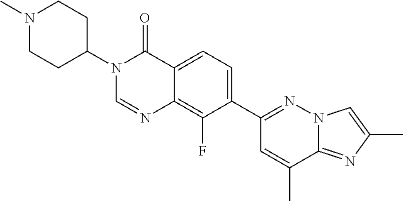 |
| 291 | 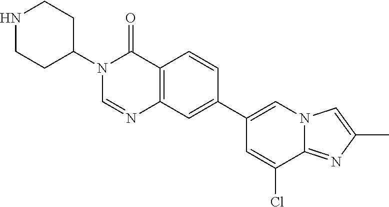 [SMILES: Cc1cn2cc(-c3ccc4c(=O)n(C5CCNCC5)cnc4c3)cc(Cl)c2n1] |
| 292 | 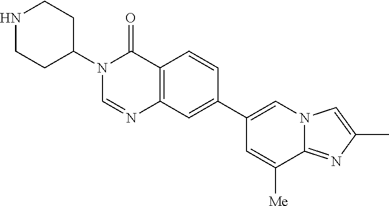 [SMILES: Cc1cn2cc(-c3ccc4c(=O)n(C5CCNCC5)cnc4c3)cc(C)c2n1] |
| 293 | 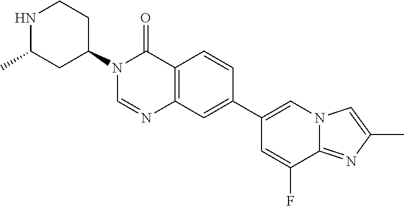 |
| 294 | 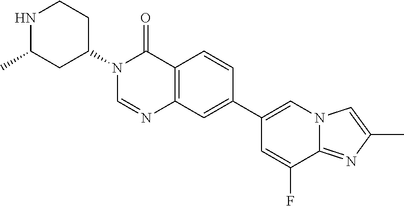 |
| 295 | 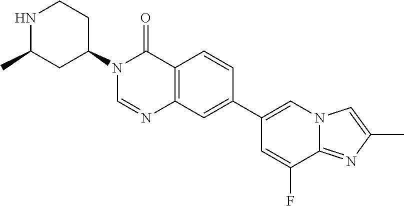 |
| 296 | 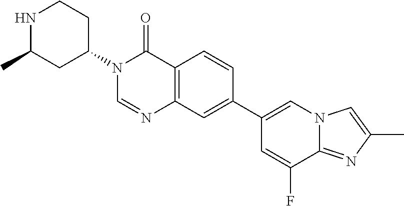 |
| 297 | 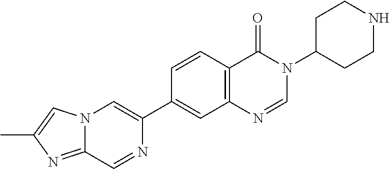 [SMILES: Cc1cn2cc(-c3ccc4c(=O)n(C5CCNCC5)cnc4c3)ncc2n1] |
| 298 | 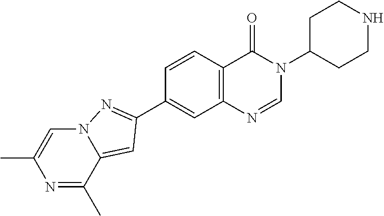 [SMILES: Cc1cn2nc(-c3ccc4c(=O)n(C5CCNCC5)cnc4c3)cc2c(C)n1] |
| 299 | 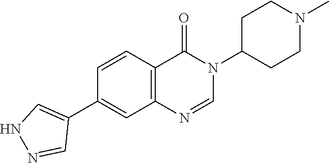 [SMILES: CN1CCC(n2cnc3cc(-c4cn[nH]c4)ccc3c2=O)CC1] |
| 300 | 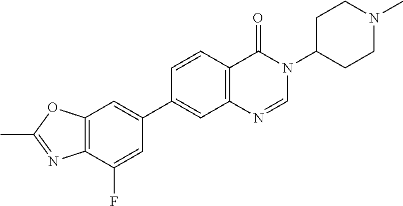 |
| 301 | 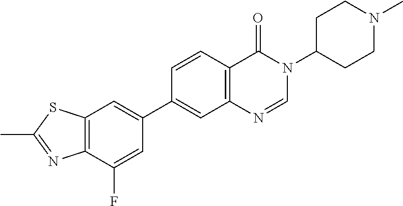 |
| 302 | 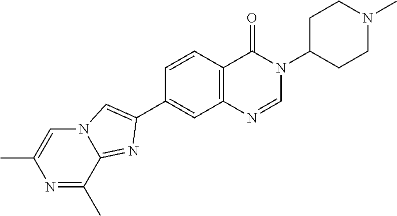 |
| 303 | 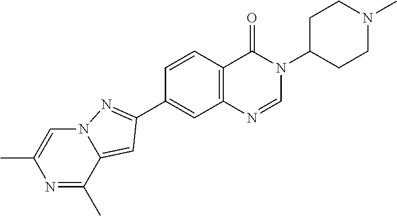 |
| 306 | 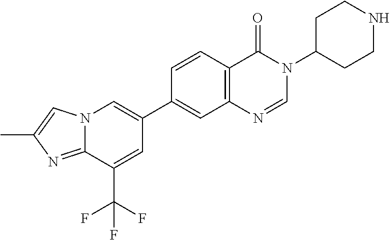 [SMILES: Cc1cn2cc(-c3ccc4c(=O)n(C5CCNCC5)cnc4c3)cc(C(F)(F)F)c2n1] |
| 307 | 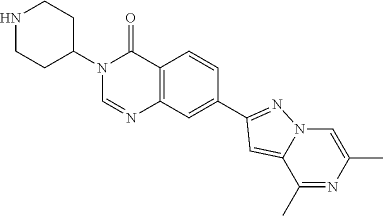 [SMILES: Cc1cn2nc(-c3ccc4c(=O)n(C5CCNCC5)cnc4c3)cc2c(C)n1] |
| 308 | 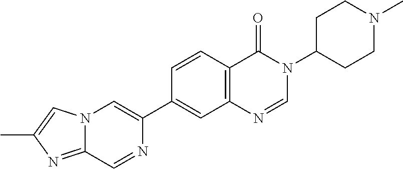 |
| 311 | 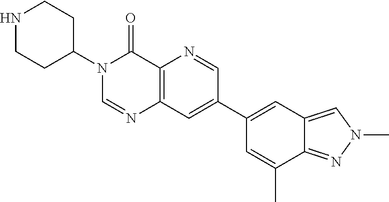 [SMILES: Cc1cc(-c2cnc3c(=O)n(C4CCNCC4)cnc3c2)cc2cn(C)nc12] |
| 313 | 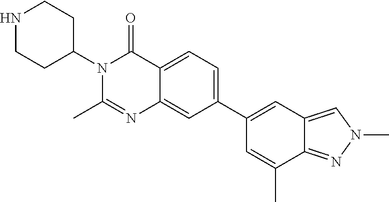 [SMILES: Cc1cc(-c2ccc3c(=O)n(C4CCNCC4)c(C)nc3c2)cc2cn(C)nc12] |
| 314 | 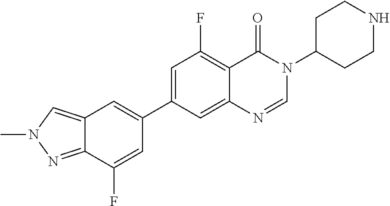 [SMILES: Cn1cc2cc(-c3cc(F)c4c(=O)n(C5CCNCC5)cnc4c3)cc(F)c2n1] |
| 315 | 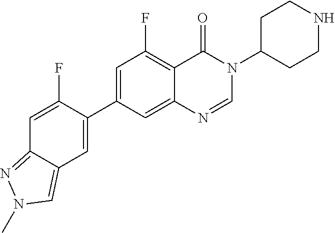 [SMILES: Cn1cc2cc(-c3cc(F)c4c(=O)n(C5CCNCC5)cnc4c3)c(F)cc2n1] |
| 316 | 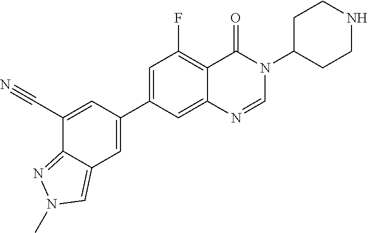 [SMILES: Cn1cc2cc(-c3cc(F)c4c(=O)n(C5CCNCC5)cnc4c3)cc(C#N)c2n1] |
| 317 | 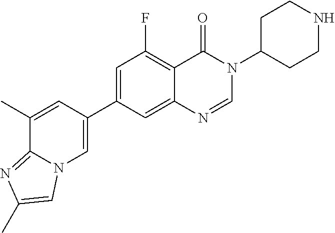 [SMILES: Cc1cn2cc(-c3cc(F)c4c(=O)n(C5CCNCC5)cnc4c3)cc(C)c2n1] |
| 318 | 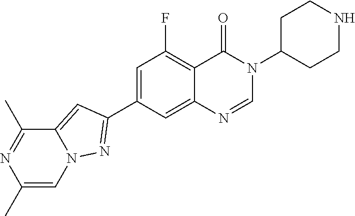 [SMILES: Cc1cn2nc(-c3cc(F)c4c(=O)n(C5CCNCC5)cnc4c3)cc2c(C)n1] |
| 319 | 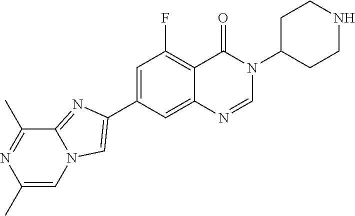 [SMILES: Cc1cn2cc(-c3cc(F)c4c(=O)n(C5CCNCC5)cnc4c3)nc2c(C)n1] |
| 320 | 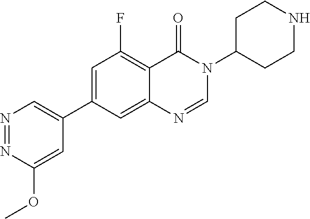 [SMILES: COc1cc(-c2cc(F)c3c(=O)n(C4CCNCC4)cnc3c2)cnn1] |
| 321 | 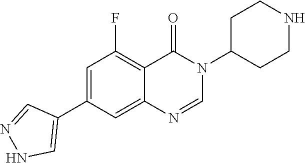 [SMILES: O=c1c2c(F)cc(-c3cn[nH]c3)cc2ncn1C1CCNCC1] |
| 323 | 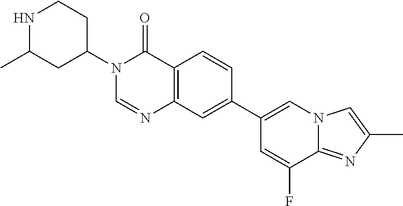 |
| 338 | 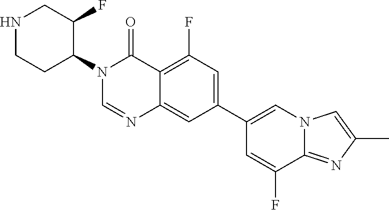 [SMILES: Cc1cn2cc(-c3cc(F)c4c(=O)n([C@H]5CCNC[C@H]5F)cnc4c3)cc(F)c2n1] |
| 339 | 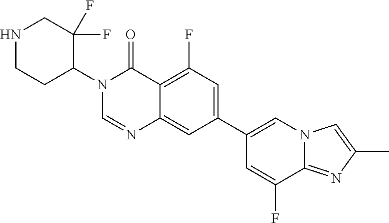 [SMILES: Cc1cn2cc(-c3cc(F)c4c(=O)n(C5CCNCC5(F)F)cnc4c3)cc(F)c2n1] |
| 340 | 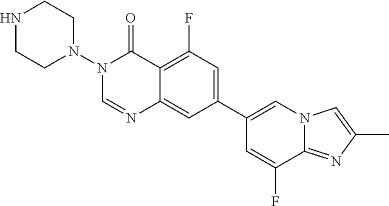 [SMILES: Cc1cn2cc(-c3cc(F)c4c(=O)n(N5CCNCC5)cnc4c3)cc(F)c2n1] |
| 341 | 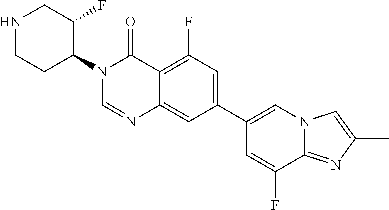 [SMILES: Cc1cn2cc(-c3cc(F)c4c(=O)n([C@H]5CCNC[C@@H]5F)cnc4c3)cc(F)c2n1] |
| 342 | 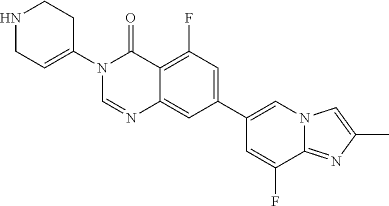 [SMILES: Cc1cn2cc(-c3cc(F)c4c(=O)n(C5=CCNCC5)cnc4c3)cc(F)c2n1] |
| 343 | 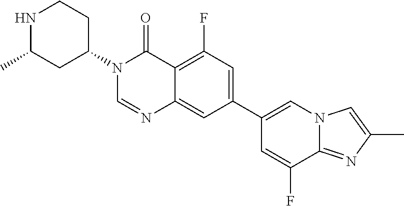 |
| 344 | 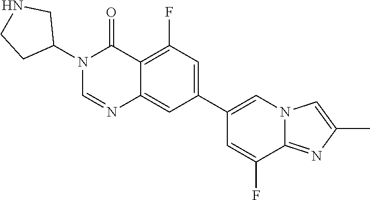 [SMILES: Cc1cn2cc(-c3cc(F)c4c(=O)n(C5CCNC5)cnc4c3)cc(F)c2n1] |
| 345 | 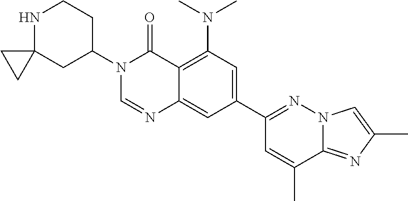 [SMILES: Cc1cn2nc(-c3cc(N(C)C)c4c(=O)n(C5CCNC6(CC6)C5)cnc4c3)cc(C)c2n1] |
| 346 | 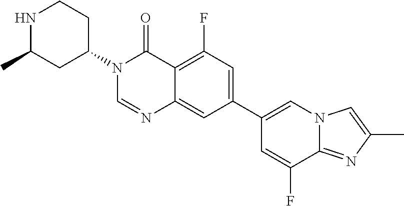 |
| 347 | 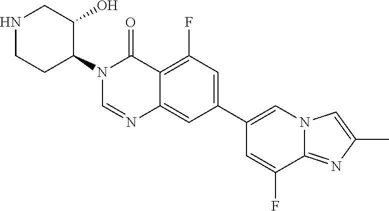 [SMILES: Cc1cn2cc(-c3cc(F)c4c(=O)n([C@H]5CCNC[C@@H]5O)cnc4c3)cc(F)c2n1] |
| 348 | 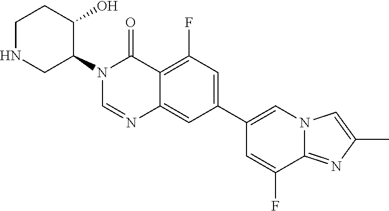 [SMILES: Cc1cn2cc(-c3cc(F)c4c(=O)n([C@H]5CNCC[C@@H]5O)cnc4c3)cc(F)c2n1] |
| 349 | 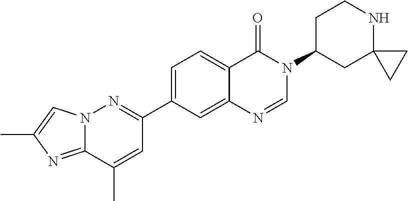 [SMILES: Cc1cn2nc(-c3ccc4c(=O)n([C@H]5CCNC6(CC6)C5)cnc4c3)cc(C)c2n1] |
| 350 | 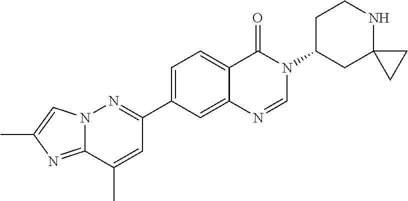 [SMILES: Cc1cn2nc(-c3ccc4c(=O)n([C@@H]5CCNC6(CC6)C5)cnc4c3)cc(C)c2n1] |
| 351 | 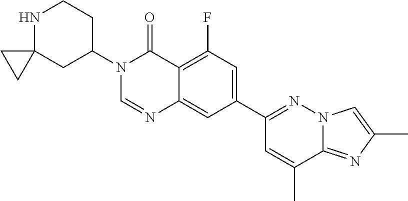 [SMILES: Cc1cn2nc(-c3cc(F)c4c(=O)n(C5CCNC6(CC6)C5)cnc4c3)cc(C)c2n1] |
| 352 | 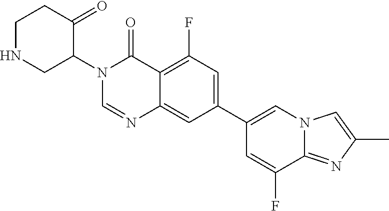 [SMILES: Cc1cn2cc(-c3cc(F)c4c(=O)n(C5CNCCC5=O)cnc4c3)cc(F)c2n1] |
| 353 | 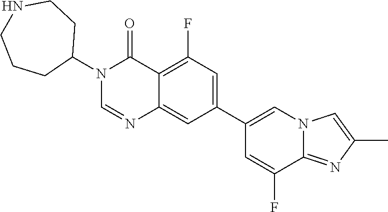 [SMILES: Cc1cn2cc(-c3cc(F)c4c(=O)n(C5CCCNCC5)cnc4c3)cc(F)c2n1] |
| 354 | 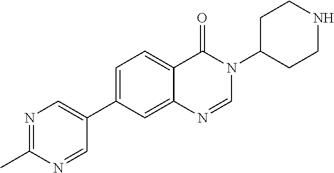 [SMILES: Cc1ncc(-c2ccc3c(=O)n(C4CCNCC4)cnc3c2)cn1] |
| 355 | 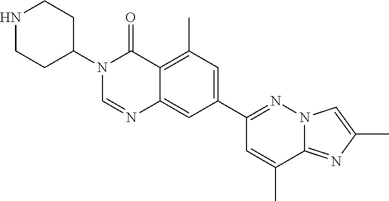 [SMILES: Cc1cn2nc(-c3cc(C)c4c(=O)n(C5CCNCC5)cnc4c3)cc(C)c2n1] |
| 356 | 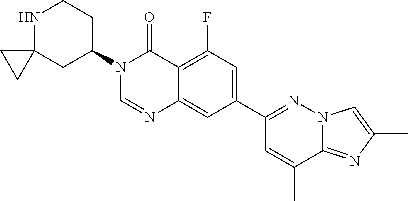 [SMILES: Cc1cn2nc(-c3cc(F)c4c(=O)n([C@@H]5CCNC6(CC6)C5)cnc4c3)cc(C)c2n1] |
| 357 | 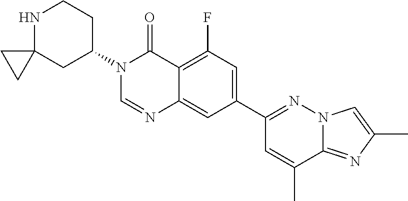 [SMILES: Cc1cn2nc(-c3cc(F)c4c(=O)n([C@H]5CCNC6(CC6)C5)cnc4c3)cc(C)c2n1] |
| 358 | 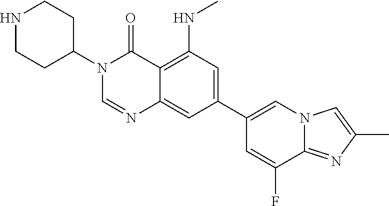 [SMILES: CNc1cc(-c2cc(F)c3nc(C)cn3c2)cc2ncn(C3CCNCC3)c(=O)c12] |
| 359 | 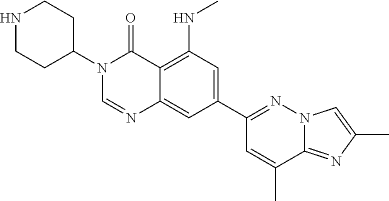 [SMILES: CNc1cc(-c2cc(C)c3nc(C)cn3n2)cc2ncn(C3CCNCC3)c(=O)c12] |
| 360 | 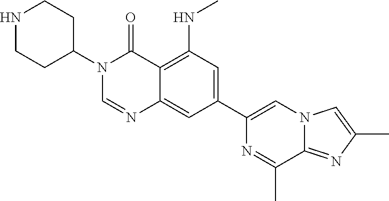 [SMILES: CNc1cc(-c2cn3cc(C)nc3c(C)n2)cc2ncn(C3CCNCC3)c(=O)c12] |
| 361 | 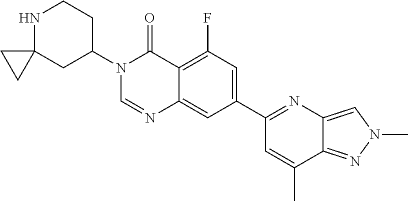 [SMILES: Cc1cc(-c2cc(F)c3c(=O)n(C4CCNC5(CC5)C4)cnc3c2)nc2cn(C)nc12] |
| 362 | 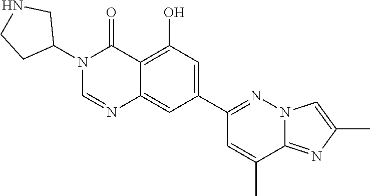 [SMILES: Cc1cn2nc(-c3cc(O)c4c(=O)n(C5CCNC5)cnc4c3)cc(C)c2n1] |
| 363 | 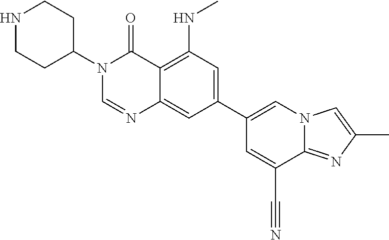 [SMILES: CNc1cc(-c2cc(C#N)c3nc(C)cn3c2)cc2ncn(C3CCNCC3)c(=O)c12] |
| 364 | 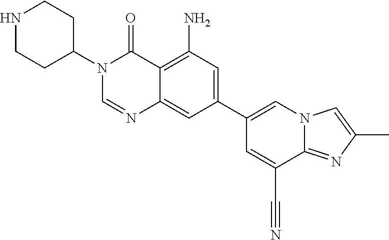 [SMILES: Cc1cn2cc(-c3cc(N)c4c(=O)n(C5CCNCC5)cnc4c3)cc(C#N)c2n1] |
| 365 | 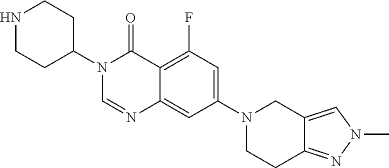 [SMILES: Cn1cc2c(n1)CCN(c1cc(F)c3c(=O)n(C4CCNCC4)cnc3c1)C2] |
| 366 | 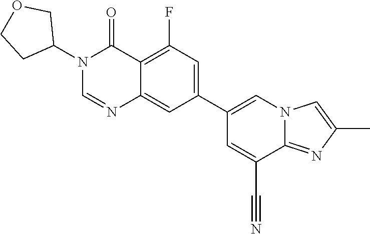 [SMILES: Cc1cn2cc(-c3cc(F)c4c(=O)n(C5CCOC5)cnc4c3)cc(C#N)c2n1] |
| 367 | 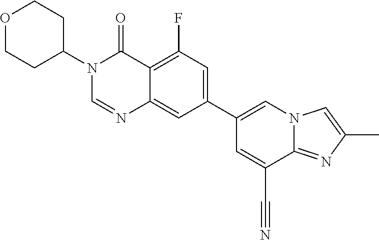 [SMILES: Cc1cn2cc(-c3cc(F)c4c(=O)n(C5CCOCC5)cnc4c3)cc(C#N)c2n1] |
| 491 | 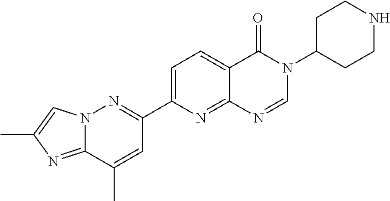 [SMILES: Cc1cn2nc(-c3ccc4c(=O)n(C5CCNCC5)cnc4n3)cc(C)c2n1] |
| 492 | 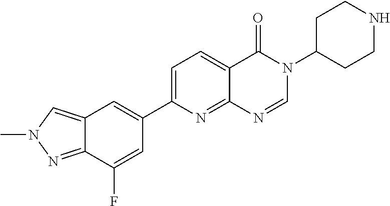 [SMILES: Cn1cc2cc(-c3ccc4c(=O)n(C5CCNCC5)cnc4n3)cc(F)c2n1] |
| 493 | 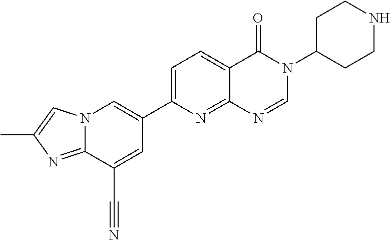 [SMILES: Cc1cn2cc(-c3ccc4c(=O)n(C5CCNCC5)cnc4n3)cc(C#N)c2n1] |
| 494 | 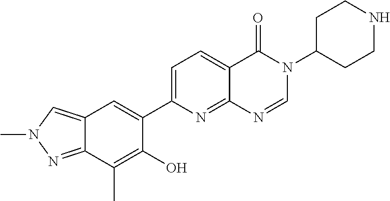 [SMILES: Cc1c(O)c(-c2ccc3c(=O)n(C4CCNCC4)cnc3n2)cc2cn(C)nc12] |
| 495 | 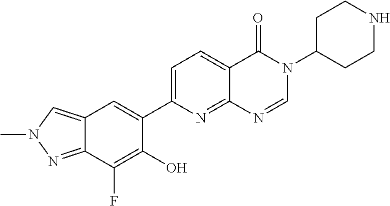 [SMILES: Cn1cc2cc(-c3ccc4c(=O)n(C5CCNCC5)cnc4n3)c(O)c(F)c2n1] |
| 496 | 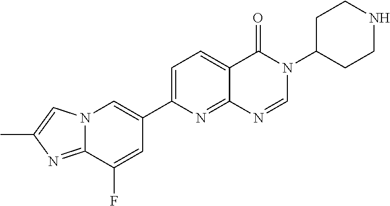 [SMILES: Cc1cn2cc(-c3ccc4c(=O)n(C5CCNCC5)cnc4n3)cc(F)c2n1] |
| 497 | 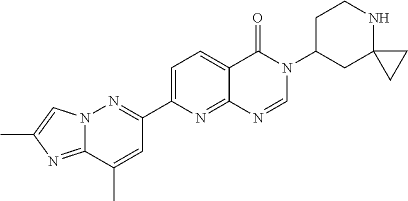 [SMILES: Cc1cn2nc(-c3ccc4c(=O)n(C5CCNC6(CC6)C5)cnc4n3)cc(C)c2n1] |
| 498 | 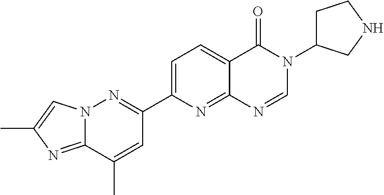 [SMILES: Cc1cn2nc(-c3ccc4c(=O)n(C5CCNC5)cnc4n3)cc(C)c2n1] |
| 499 | 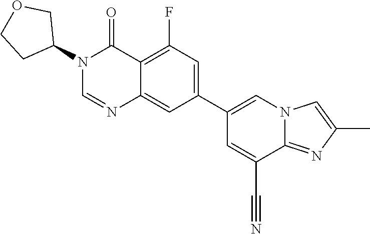 [SMILES: Cc1cn2cc(-c3cc(F)c4c(=O)n([C@H]5CCOC5)cnc4c3)cc(C#N)c2n1] |
| 500 | 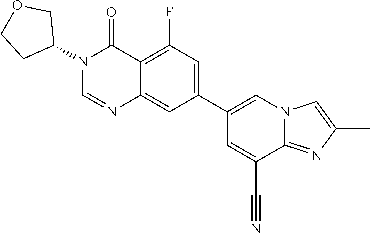 [SMILES: Cc1cn2cc(-c3cc(F)c4c(=O)n([C@@H]5CCOC5)cnc4c3)cc(C#N)c2n1] |
| 501 | 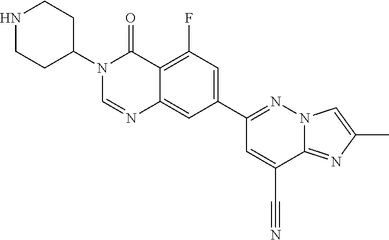 [SMILES: Cc1cn2nc(-c3cc(F)c4c(=O)n(C5CCNCC5)cnc4c3)cc(C#N)c2n1] |
| 502 | 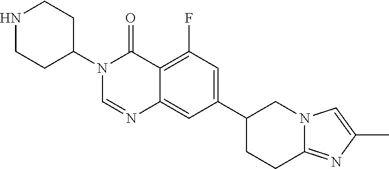 [SMILES: Cc1cn2c(n1)CCC(c1cc(F)c3c(=O)n(C4CCNCC4)cnc3c1)C2] |
| 503 | 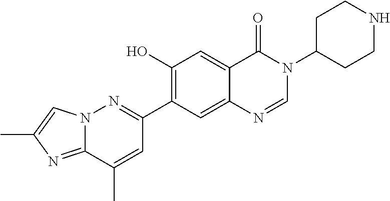 [SMILES: Cc1cn2nc(-c3cc4ncn(C5CCNCC5)c(=O)c4cc3O)cc(C)c2n1] |
| 504 | 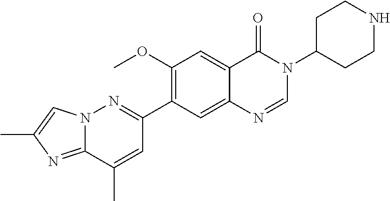 [SMILES: COc1cc2c(=O)n(C3CCNCC3)cnc2cc1-c1cc(C)c2nc(C)cn2n1] |
| 505 | 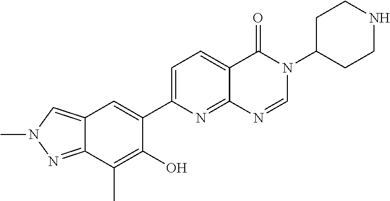 [SMILES: Cc1c(O)c(-c2ccc3c(=O)n(C4CCNCC4)cnc3n2)cc2cn(C)nc12] |
| 506 | 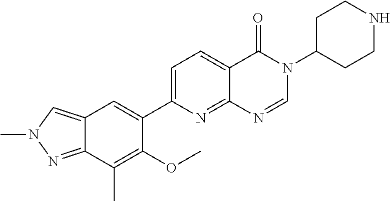 [SMILES: COc1c(-c2ccc3c(=O)n(C4CCNCC4)cnc3n2)cc2cn(C)nc2c1C] |
| 507 | 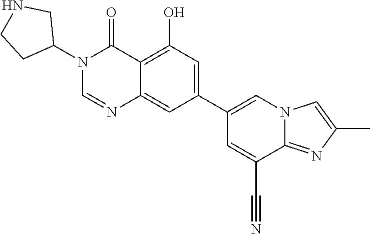 [SMILES: Cc1cn2cc(-c3cc(O)c4c(=O)n(C5CCNC5)cnc4c3)cc(C#N)c2n1] |
| 508 | 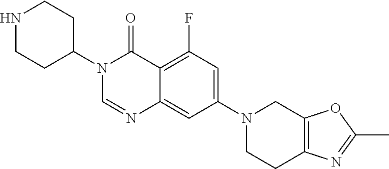 [SMILES: Cc1nc2c(o1)CN(c1cc(F)c3c(=O)n(C4CCNCC4)cnc3c1)CC2] |
| 585 | 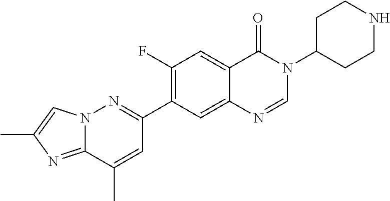 [SMILES: Cc1cn2nc(-c3cc4ncn(C5CCNCC5)c(=O)c4cc3F)cc(C)c2n1] |
| 586 | 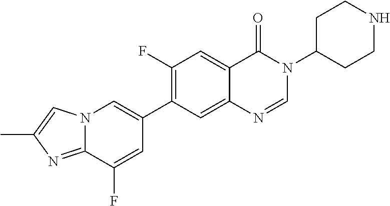 [SMILES: Cc1cn2cc(-c3cc4ncn(C5CCNCC5)c(=O)c4cc3F)cc(F)c2n1] |
| 587 | 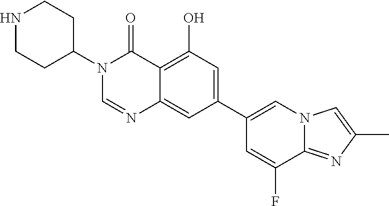 [SMILES: Cc1cn2cc(-c3cc(O)c4c(=O)n(C5CCNCC5)cnc4c3)cc(F)c2n1] |
| 588 | 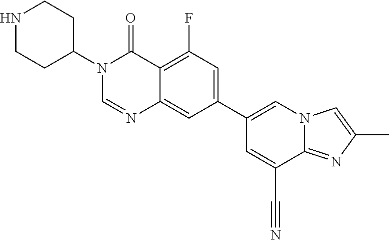 [SMILES: Cc1cn2cc(-c3cc(F)c4c(=O)n(C5CCNCC5)cnc4c3)cc(C#N)c2n1] |
| 589 | 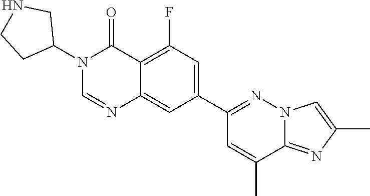 [SMILES: Cc1cn2nc(-c3cc(F)c4c(=O)n(C5CCNC5)cnc4c3)cc(C)c2n1] |
| 590 | 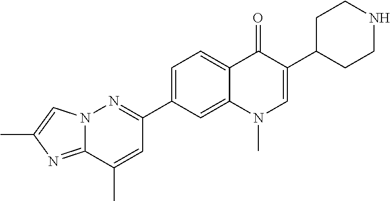 [SMILES: Cc1cn2nc(-c3ccc4c(=O)c(C5CCNCC5)cn(C)c4c3)cc(C)c2n1] |
| 591 | 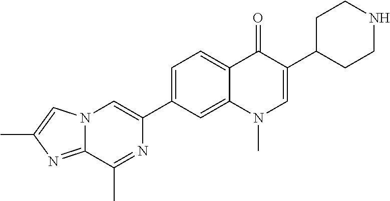 [SMILES: Cc1cn2cc(-c3ccc4c(=O)c(C5CCNCC5)cn(C)c4c3)nc(C)c2n1] |
| 592 | 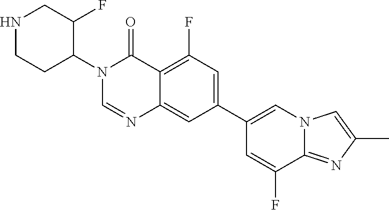 [SMILES: Cc1cn2cc(-c3cc(F)c4c(=O)n(C5CCNCC5F)cnc4c3)cc(F)c2n1] |
| 593 | 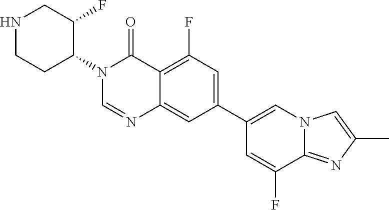 [SMILES: Cc1cn2cc(-c3cc(F)c4c(=O)n([C@@H]5CCNC[C@@H]5F)cnc4c3)cc(F)c2n1] |
| 594 | 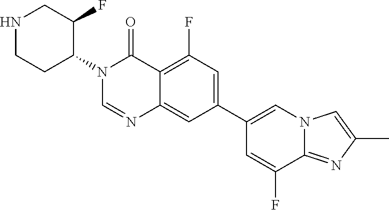 [SMILES: Cc1cn2cc(-c3cc(F)c4c(=O)n([C@@H]5CCNC[C@H]5F)cnc4c3)cc(F)c2n1] |
| 595 | 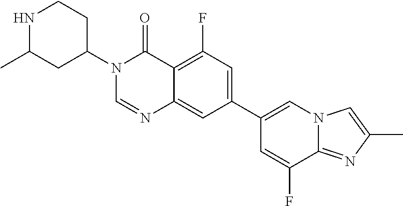 |
| 596 | 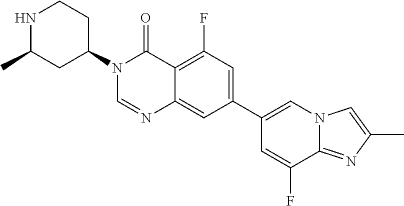 |
| 597 | 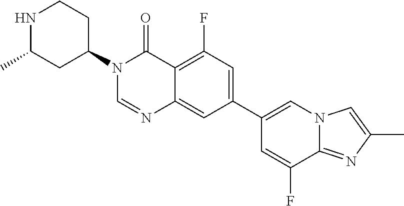 |
| 598 | 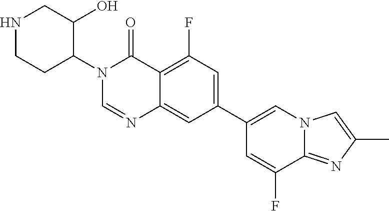 [SMILES: Cc1cn2cc(-c3cc(F)c4c(=O)n(C5CCNCC5O)cnc4c3)cc(F)c2n1] |
| 599 | 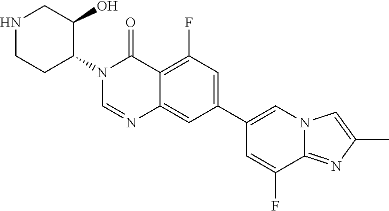 [SMILES: Cc1cn2cc(-c3cc(F)c4c(=O)n([C@@H]5CCNC[C@H]5O)cnc4c3)cc(F)c2n1] |
| 600 | 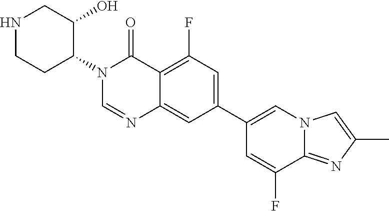 [SMILES: Cc1cn2cc(-c3cc(F)c4c(=O)n([C@@H]5CCNC[C@@H]5O)cnc4c3)cc(F)c2n1] |
| 601 | 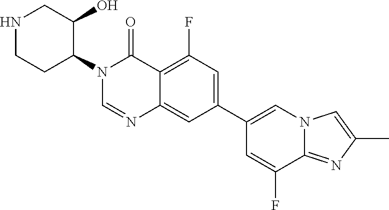 [SMILES: Cc1cn2cc(-c3cc(F)c4c(=O)n([C@H]5CCNC[C@H]5O)cnc4c3)cc(F)c2n1] |
| 602 | 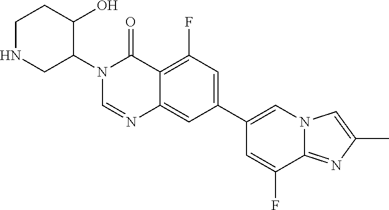 [SMILES: Cc1cn2cc(-c3cc(F)c4c(=O)n(C5CNCCC5O)cnc4c3)cc(F)c2n1] |
| 603 | 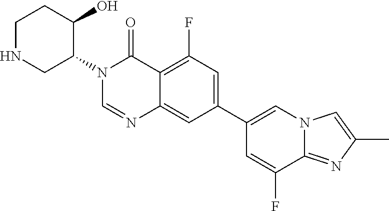 [SMILES: Cc1cn2cc(-c3cc(F)c4c(=O)n([C@@H]5CNCC[C@H]5O)cnc4c3)cc(F)c2n1] |
| 604 | 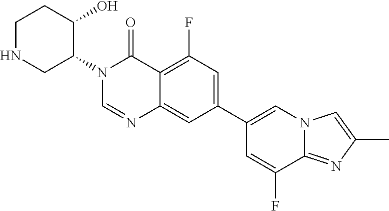 [SMILES: Cc1cn2cc(-c3cc(F)c4c(=O)n([C@@H]5CNCC[C@@H]5O)cnc4c3)cc(F)c2n1] |
| 605 | 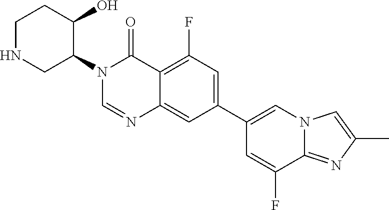 [SMILES: Cc1cn2cc(-c3cc(F)c4c(=O)n([C@H]5CNCC[C@H]5O)cnc4c3)cc(F)c2n1] |
| 606 | 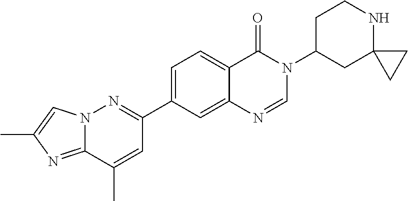 [SMILES: Cc1cn2nc(-c3ccc4c(=O)n(C5CCNC6(CC6)C5)cnc4c3)cc(C)c2n1] |
| 607 | 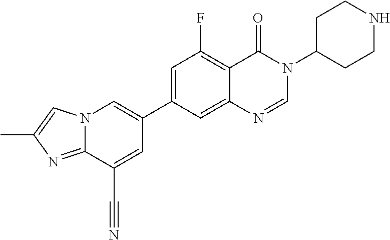 [SMILES: Cc1cn2cc(-c3cc(F)c4c(=O)n(C5CCNCC5)cnc4c3)cc(C#N)c2n1] |
| 608 | 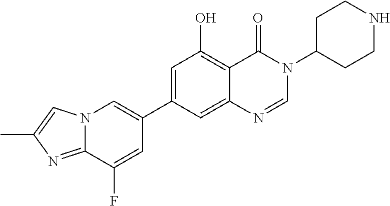 [SMILES: Cc1cn2cc(-c3cc(O)c4c(=O)n(C5CCNCC5)cnc4c3)cc(F)c2n1] |
| 609 | 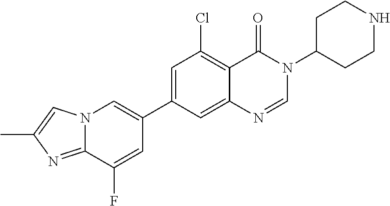 [SMILES: Cc1cn2cc(-c3cc(Cl)c4c(=O)n(C5CCNCC5)cnc4c3)cc(F)c2n1] |
| 657 | 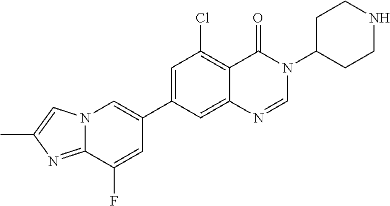 [SMILES: Cc1cn2cc(-c3cc(Cl)c4c(=O)n(C5CCNCC5)cnc4c3)cc(F)c2n1] |

In some embodiments, for Formula (III), A is monocyclic heterocyclyl (e.g., N-methyl piperazyl); B is bicyclic heteroaryl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 152, or a pharmaceutically acceptable salt, solvate, hydrate, tautomer, or stereoisomer thereof.

In some embodiments, for Formula (III), A is monocyclic heterocyclyl (e.g., N-methyl piperazyl); B is bicyclic heteroaryl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 153, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 156, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., 2,2,6,6-tetramethylpiperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 157, or a pharmaceutically acceptable salt, solvate, hydrate, tautomer, or stereoisomer thereof.

In some embodiments, for Formula (III), A is monocyclic heterocyclyl (e.g., 1,2,3,6-tetrahydropyridinyl); B is bicyclic heteroaryl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 158, or a pharmaceutically acceptable salt, solvate, hydrate, tautomer, or stereoisomer thereof.

In some embodiments, for Formula (III), A is monocyclic heterocyclyl (e.g., N-methyl 1,2,3,6-tetrahydropyridinyl); B is bicyclic heteroaryl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 159, or a pharmaceutically acceptable salt, solvate, hydrate, tautomer, or stereoisomer thereof.

In some embodiments, for Formula (III), A is bicyclic heterocyclyl (e.g., 8-azabicyclo[3.2.1]oct-2-enyl); B is bicyclic heteroaryl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 160, or a pharmaceutically acceptable salt, solvate, hydrate, tautomer, or stereoisomer thereof.

In some embodiments, for Formula (III), A is bicyclic heterocyclyl (e.g., N-methyl 8-azabicyclo[3.2.1]oct-2-enyl); B is bicyclic heteroaryl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 161, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 2-methyl-2H-indazolyl); L¹ is —N(R⁸)—(e.g., —NH—); L² are absent; X and Z are each independently C(R³) (e.g., CH); Y is N; R² is absent; and R^{7a} and R^{7b} are each independently hydrogen. In some embodiments, the compound of Formulas (III) and (III-a) is Compound 162, or a pharmaceutically acceptable salt, solvate, hydrate, tautomer, or stereoisomer thereof.

In some embodiments, for Formula (III), A is monocyclic heterocyclyl (e.g., N-methyl piperidinyl); B is bicyclic heteroaryl (e.g., 2-methyl-2H-indazolyl); L¹ is —N(R⁸)—(e.g., —NH—); L² are absent; X and Z are each independently C(R³) (e.g., CH); Y is N; R² is absent; and R^{7a} and R^{7b} are each independently hydrogen. In some embodiments, the compound of Formulas (III) and (III-a) is Compound 163, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 172, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., N-methyl piperidinyl); L¹ and L² are absent; X is C(R³) (e.g., CH); Z and Y are each independently N; R² is absent; and R^{7a} and R^{7b} are each independently hydrogen. In some embodiments, the compound of Formulas (III), (III-a), and (III-b) is Compound 173, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 174, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., N-methyl piperazyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 175, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 176, or a pharmaceutically acceptable salt, solvate, hydrate, tautomer, or stereoisomer thereof.

In some embodiments, for Formula (III), A is monocyclic heterocyclyl (e.g., N-methyl piperidinyl); B is bicyclic heteroaryl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 177, or a pharmaceutically acceptable salt, solvate, hydrate, tautomer, or stereoisomer thereof.

In some embodiments, for Formula (III), A is monocyclic heterocyclyl (e.g., N-methyl piperazyl); B is bicyclic heteroaryl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 178, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., 2,2,6,6-tetramethylpiperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-b) is Compound 179, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 2-methyl-2H-indazolyl); L¹ is —N(R⁸)—(e.g., —N(CH₃)—); L² is absent; X and Z are each independently C(R³) (e.g., CH); Y is N; R² is absent; and R^{7a} and R^{7b} are each independently hydrogen. In some embodiments, the compound of Formulas (III) and (III-a) is Compound 180, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 181, or a pharmaceutically acceptable salt, solvate, hydrate, tautomer, or stereoisomer thereof.

In some embodiments, for Formula (III), A is bicyclic heteroaryl (e.g., 2-methyl-2H-indazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 182, or a pharmaceutically acceptable salt, solvate, hydrate, tautomer, or stereoisomer thereof.

In some embodiments, for Formula (III), A is monocyclic heterocyclyl (e.g., N-methyl piperidinyl); B is bicyclic heteroaryl (e.g., 2-methyl-2H-indazolyl); L¹ is —N(R⁸)—(e.g., —NH—); L² is absent; X and Z are each independently C(R³) (e.g., CH); Y is N; R² is absent; and R^{7a} and R^{7b} are each independently hydrogen. In some embodiments, the compound of Formulas (III) and (III-a) is Compound 203, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 2-methyl-2H-indazolyl); L¹ is —N(R⁸)—(e.g., —NH—); L² is absent; X and Z are each independently C(R³) (e.g., CH); Y is N; R² is absent; and R^{7a} and R^{7b} are each independently hydrogen. In some embodiments, the compound of Formulas (III) and (III-a) is Compound 204, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 205, or a pharmaceutically acceptable salt, solvate, hydrate, tautomer, or stereoisomer thereof.

In some embodiments, for Formula (III), A is bicyclic heteroaryl (e.g., 7-fluoro-2-methyl-2H-indazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 206, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 207, or a pharmaceutically acceptable salt, solvate, hydrate, tautomer, or stereoisomer thereof.

In some embodiments, for Formula (III), A is bicyclic heteroaryl (e.g., 2-methyl-2H-indazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 208, or a pharmaceutically acceptable salt, solvate, hydrate, tautomer, or stereoisomer thereof.

In some embodiments, for Formula (III), A is bicyclic heteroaryl (e.g., 7-fluoro-2-methyl-2H-indazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 209, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., piperidinyl); L¹ and L² are absent; X is C(R³) (e.g., CH); Z is C(R³) (e.g., CF); Y is N; R² is absent; and R^{7a} and R^{7b} are each independently hydrogen. In some embodiments, the compound of Formulas (III) and (III-a) is Compound 210, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., N-methyl piperidinyl); L¹ and L² are absent; X is C(R³) (e.g., CH); Z is C(R³) (e.g., CF); Y is N; R² is absent; and R^{7a} and R^{7b} are each independently hydrogen. In some embodiments, the compound of Formulas (III) and (III-a) is Compound 227, or a pharmaceutically acceptable salt, solvate, hydrate, tautomer, or stereoisomer thereof.

In some embodiments, for Formula (III), A is monocyclic heterocyclyl (e.g., piperazyl); B is bicyclic heteroaryl (e.g., 8-fluoro-2-methylimidazo[1,2-a]pyridinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 228, or a pharmaceutically acceptable salt, solvate, hydrate, tautomer, or stereoisomer thereof.

In some embodiments, for Formula (III), A is monocyclic heterocyclyl (e.g., N-methyl piperazyl); B is bicyclic heteroaryl (e.g., 8-fluoro-2-methylimidazo[1,2-a]pyridinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 229, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-c) is Compound 230, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., N-methyl piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-c) is Compound 231, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-c) is Compound 232, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., N-methyl piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-c) is Compound 233, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., piperidinyl); L¹ and L² are absent; X is C(R³) (e.g., Ch); Z is C(R³) (e.g., CF); Y is N; R² is absent; and R^{7a} and R^{7b} are each independently hydrogen. In some embodiments, the compound of Formulas (III) and (III-a) is Compound 234, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., N-methyl piperidinyl); L¹ and L² are absent; X is C(R³) (e.g., CH); Z is C(R³) (e.g., CF); Y is N; R² is absent; and R^{7a} and R^{7b} are each independently hydrogen. In some embodiments, the compound of Formulas (III) and (III-a) is Compound 235, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is bicyclic heterocyclyl (e.g., 4-azaspiro[2.5]octanyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 236, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 2,2-dimethylpiperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 237, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-c) is Compound 240, or a pharmaceutically acceptable salt, solvate, hydrate, tautomer, or stereoisomer thereof.

In some embodiments, for Formula (III), A is monocyclic heterocyclyl (e.g., piperazyl); B is bicyclic heteroaryl (e.g., 2,8-dimethylimidazo[1,2-b]pyridazyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 241, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., N-methyl piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-c) is Compound 242, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-c) is Compound 243, or a pharmaceutically acceptable salt, solvate, hydrate, tautomer, or stereoisomer thereof.

In some embodiments, for Formula (III), A is bicyclic heteroaryl (e.g., 2,7-dimethylimidazo[1,2-a]pyridinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 244, or a pharmaceutically acceptable salt, solvate, hydrate, tautomer, or stereoisomer thereof.

In some embodiments, for Formula (III), A is bicyclic heteroaryl (e.g., 4-fluoro-2-methylbenzo[d]oxazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 245, or a pharmaceutically acceptable salt, solvate, hydrate, tautomer, or stereoisomer thereof.

In some embodiments, for Formula (III), A is bicyclic heteroaryl (e.g., 2,7-dimethylimidazo[1,2-a]pyridinyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 246, or a pharmaceutically acceptable salt, solvate, hydrate, tautomer, or stereoisomer thereof.

In some embodiments, for Formula (III), A is monocyclic heteroaryl (e.g., pyrazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 284, or a pharmaceutically acceptable salt, solvate, hydrate, tautomer, or stereoisomer thereof.

In some embodiments, for Formula (III), A is bicyclic heteroaryl (e.g., 4-fluoro-2-methylbenzo[d]thiazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 285, or a pharmaceutically acceptable salt, solvate, hydrate, tautomer, or stereoisomer thereof.

In some embodiments, for Formula (III), A is bicyclic heteroaryl (e.g., 6,8-dimethylimidazo[1,2-a]pyrazyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 286, or a pharmaceutically acceptable salt, solvate, hydrate, tautomer, or stereoisomer thereof.

In some embodiments, for Formula (III), A is bicyclic heteroaryl (e.g., 6,8-dimethyl-[1,2,4]triazolo[1,5-a]pyrazinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 287, or a pharmaceutically acceptable salt, solvate, hydrate, tautomer, or stereoisomer thereof.

In some embodiments, for Formula (III), A is bicyclic heteroaryl (e.g., 6,8-dimethyl-[1,2,4]triazolo[1,5-a]pyrazinyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 288, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., piperidinyl); L¹ and L² are absent; X is C(R³) (e.g., CH); Z is C(R³) (e.g., CF); Y is N; R² is absent; and R^{7a} and R^{7b} are each independently hydrogen. In some embodiments, the compound of Formulas (III), (III-a), and (III-c) is Compound 289, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., N-methyl piperidinyl); L¹ and L² are absent; X is C(R³) (e.g., CH); Z is C(R³) (e.g., CF); Y is N; R² is absent; and R^{7a} and R^{7b} are each independently hydrogen. In some embodiments, the compound of Formulas (III), (III-a), and (III-c) is Compound 290, or a pharmaceutically acceptable salt, solvate, hydrate, tautomer, or stereoisomer thereof.

In some embodiments, for Formula (III), A is bicyclic heteroaryl (e.g., 8-chloro-2-methylimidazo[1,2-a]pyridinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 291, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-a]pyridinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 292, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 2-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 293, 294, 295, 296, or 323, or a pharmaceutically acceptable salt, solvate, hydrate, tautomer, or stereoisomer thereof.

In some embodiments, for Formula (III), A is bicyclic heteroaryl (e.g., 2-methylimidazo[1,2-a]pyrazyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 297, or a pharmaceutically acceptable salt, solvate, hydrate, tautomer, or stereoisomer thereof.

In some embodiments, for Formula (III), A is bicyclic heteroaryl (e.g., 4,6-dimethylpyrazolo[1,5-a]pyrazyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 298, or a pharmaceutically acceptable salt, solvate, hydrate, tautomer, or stereoisomer thereof.

In some embodiments, for Formula (III), A is monocyclic heteroaryl (e.g., pyrazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 299, or a pharmaceutically acceptable salt, solvate, hydrate, tautomer, or stereoisomer thereof.

In some embodiments, for Formula (III), A is bicyclic heteroaryl (e.g., 4-fluoro-2-methylbenzo[d]oxazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 300, or a pharmaceutically acceptable salt, solvate, hydrate, tautomer, or stereoisomer thereof.

In some embodiments, for Formula (III), A is bicyclic heteroaryl (e.g., 4-fluoro-2-methylbenzo[d]thiazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 301, or a pharmaceutically acceptable salt, solvate, hydrate, tautomer, or stereoisomer thereof.

In some embodiments, for Formula (III), A is bicyclic heteroaryl (e.g., 6,8-dimethylimidazo[1,2-a]pyrazyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 302, or a pharmaceutically acceptable salt, solvate, hydrate, tautomer, or stereoisomer thereof.

In some embodiments, for Formula (III), A is bicyclic heteroaryl (e.g., 4,6-dimethylpyrazolo[1,5-a]pyrazyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 303, or a pharmaceutically acceptable salt, solvate, hydrate, tautomer, or stereoisomer thereof.

In some embodiments, for Formula (III), A is bicyclic heteroaryl (e.g., 2-methyl-8-(trifluoromethyl)imidazo[1,2-a]pyridinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formula (III), (III-a), and (III-c) is Compound 306, or a pharmaceutically acceptable salt, solvate, hydrate, tautomer, or stereoisomer thereof.

In some embodiments, for Formula (III), A is bicyclic heteroaryl (e.g., 4,6-dimethylpyrazolo[1,5-a]pyrazyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 307, or a pharmaceutically acceptable salt, solvate, hydrate, tautomer, or stereoisomer thereof.

In some embodiments, for Formula (III), A is bicyclic heteroaryl (e.g., 2-methylimidazo[1,2-a]pyrazyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), and (III-c) is Compound 308, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., piperidinyl); L¹ and L² are absent; X is N; Y is N; Z is C(R³) (e.g., CH); R² is absent; and R^{7a} and R^{7b} are each independently hydrogen. In some embodiments, the compound of Formula (III) and (III-a) is Compound 311, or a pharmaceutically acceptable salt, solvate, hydrate, tautomer, or stereoisomer thereof.

In some embodiments, for Formula (III), A is bicyclic heteroaryl (e.g., 2,7-dimethyl-2H-indazolyl); B is monocyclic heterocyclyl (e.g., piperidinyl); L¹ and L² are absent; X and Z are independently C(R³) (e.g., CH); Y is N; R² is absent; and R^{7a} is methyl; and R^{7b} is hydrogen. In some embodiments, the compound of Formula (III), (III-a), and (III-c) is Compound 313, or a pharmaceutically acceptable salt, solvate, hydrate, tautomer, or stereoisomer thereof.

In some embodiments, for Formula (III), A is bicyclic heteroaryl (e.g., 7-fluoro-2-methyl-2H-indazolyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 314, or a pharmaceutically acceptable salt, solvate, hydrate, tautomer, or stereoisomer thereof.

In some embodiments, for Formula (III), A is bicyclic heteroaryl (e.g., 6-fluoro-2-methyl-2H-indazolyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formulas (III), (III-a), and (III-c) is Compound 315, or a pharmaceutically acceptable salt, solvate, hydrate, tautomer, or stereoisomer thereof.

In some embodiments, for Formula (III), A is bicyclic heteroaryl (e.g., 7-cyano-2-methyl-2H-indazolyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 316, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-a]pyridinyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 317, or a pharmaceutically acceptable salt, solvate, hydrate, tautomer, or stereoisomer thereof.

In some embodiments, for Formula (III), A is bicyclic heteroaryl (e.g., 4,6-dimethylpyrazolo[1,5-a]pyrazinyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 318, or a pharmaceutically acceptable salt, solvate, hydrate, tautomer, or stereoisomer thereof.

In some embodiments, for Formula (III), A is bicyclic heterocyclyl (e.g., 6,8-dimethylimidazo[1,2-a]pyrazinyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 319, or a pharmaceutically acceptable salt, solvate, hydrate, tautomer, or stereoisomer thereof.

In some embodiments, for Formula (III), A is bicyclic heteroaryl (e.g., 3-methoxypyridazinyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 320, or a pharmaceutically acceptable salt, solvate, hydrate, tautomer, or stereoisomer thereof.

In some embodiments, for Formula (III), A is mono cyclic heteroaryl (e.g., pyrazolyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 321, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 2-methylpiperidinyl); L¹ and L² are absent; X and Z are independently C(R³) (e.g., CH); Y is N; R² is absent; and R^{7a} and R^{7b} are each independently hydrogen. In some embodiments, the compound of Formula (III), (III-a), and (III-c) is Compound 323, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 3-fluoropiperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 338, Compound 341, 592, 593, 594 or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 3,3-difluoropiperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 339, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., piperazinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 340, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 1,2,3,6-tetrahydropyridinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 342, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 2-methylpiperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 343, 595, 596, 597 or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., pyrrolidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 344, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridinyl); B is bicyclic heterocyclyl (e.g., 4-azaspiro[2.5]octanyl); L¹ and L² are absent; X is C(R³) (e.g., CN(CH₃)₂); Y is N; Z is C(R³) (e.g., CH); R² is absent; and R^{7a} and R^{7b} are each independently hydrogen. In some embodiments, the compound of Formula (III), (III-a), and (III-c) is Compound 345, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 1-methylpiperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formula (III), (III-a), and (III-c) is Compound 346, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 3-hydroxypiperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 347, 598, 599, 600, 601 or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 4-hydroxypiperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 348, 602, 603, 604, 605, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; X and Z are independently C(R³) (e.g., CH); Y is N; R² is absent; and R^{7a} and R^{7b} are each independently hydrogen. In some embodiments, the compound of Formula (III), (III-a), and (III-c) is Compound 349, Compound 350, 606, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 351, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., 4-piperidonyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 352, or a pharmaceutically acceptable salt, solvate, hydrate, tautomer, or stereoisomer thereof.

In some embodiments, for Formula (III), A is bicyclic heterocyclyl (e.g., 8-fluoro-2-methylimidazo[1,2-a]pyridinyl); B is monocyclic heterocyclyl (e.g., azepanyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 353, or a pharmaceutically acceptable salt, solvate, hydrate, tautomer, or stereoisomer thereof.

In some embodiments, for Formula (III), A is monocyclic heteroaryl (e.g., 2-methylpyrimidine); B is monocyclic heterocyclyl (e.g., piperidinyl); L¹ and L² are absent; X and Z are independently C(R³) (e.g., CH); Y is N; R² is absent; and R^{7a} and R^{7b} are each independently hydrogen. In some embodiments, the compound of Formula (III), (III-a), and (III-c) is Compound 354, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., piperidinyl); L¹ and L² are absent; X is C(R³) (e.g., CCH₃); Y is N; Z is C(R³) (e.g., CH); R² is absent; and R^{7a} and R^{7b} are each independently hydrogen. In some embodiments, the compound of Formula (III), (III-a), and (III-c) is Compound 355, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 356, Compound 357, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., piperidinyl); L¹ and L² are absent; X is C(R³) (e.g., CNHCH₃); Y is N; Z is C(R³) (e.g., CH); R² is absent; and R^{7a} and R^{7b} are each independently hydrogen. In some embodiments, the compound of Formula (III), (III-a), and (III-c) is Compound 358, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., piperidinyl); L¹ and L² are absent; X is C(R³) (e.g., CNHCH₃); Y is N; Z is C(R³) (e.g., CH); R² is absent; and R^{7a} and R^{7b} are each independently hydrogen. In some embodiments, the compound of Formula (III), (III-a), and (III-c) is Compound 359, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-a]pyrazinyl); B is monocyclic heterocyclyl (e.g., piperidinyl); L¹ and L² are absent; X is C(R³) (e.g., CNHCH₃); Y is N; Z is C(R³) (e.g., CH); R² is absent; and R^{7a} and R^{7b} are each independently hydrogen. In some embodiments, the compound of Formula (III), (III-a), and (III-c) is Compound 360, or a pharmaceutically acceptable salt, solvate, hydrate, tautomer, or stereoisomer thereof.

In some embodiments, for Formula (III), A is bicyclic heterocyclyl (e.g., 2,7-dimethylpyrazolo[4,3-b]pyridinyl); B is bicyclic heterocyclyl (e.g., 4-azaspiro[2.5]octanyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 361, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-a]pyridazyl); B is monocyclic heterocyclyl (e.g., pyrrolidinyl); L¹ and L² are absent; X is C(R³) (e.g., COH); Y is N; Z is C(R³) (e.g., CH); R² is absent; and R^{7a} and R^{7b} are each independently hydrogen. In some embodiments, the compound of Formula (III), (III-a), and (III-c) is Compound 362, or a pharmaceutically acceptable salt, solvate, hydrate, tautomer, or stereoisomer thereof.

In some embodiments, for Formula (III), A is bicyclic heteroaryl (e.g., 8-cyano-2-methylimidazo[1,2-a]pyridinyl); B is monocyclic heterocyclyl (e.g., piperidinyl); L¹ and L² are absent; X is C(R³) (e.g., CNHCH₃); Y is N; Z is C(R³) (e.g., CH); R² is absent; and R^{7a} and R^{7b} are each independently hydrogen. In some embodiments, the compound of Formula (III), (III-a), and (III-c) is Compound 363, or a pharmaceutically acceptable salt, solvate, hydrate, tautomer, or stereoisomer thereof.

In some embodiments, for Formula (III), A is bicyclic heteroaryl (e.g., 8-cyano-2-methylimidazo[1,2-a]pyridinyl); B is monocyclic heterocyclyl (e.g., piperidinyl); L¹ and L² are absent; X is C(R³) (e.g., CNH₂); Y is N; Z is C(R³) (e.g., CH); R² is absent; and R^{7a} and R^{7b} are each independently hydrogen. In some embodiments, the compound of Formula (III), (III-a), and (III-c) is Compound 364, or a pharmaceutically acceptable salt, solvate, hydrate, tautomer, or stereoisomer thereof.

In some embodiments, for Formula (III), A is bicyclic heterocyclyl (e.g., 2-methyl-4,5,6,7-tetrahydro-2H-pyrazolo[4,3-c]pyridinyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 365, or a pharmaceutically acceptable salt, solvate, hydrate, tautomer, or stereoisomer thereof.

In some embodiments, for Formula (III), A is bicyclic heteroaryl (e.g., 8-cyano-2-methylimidazo[1,2-a]pyridinyl); B is monocyclic heterocyclyl (e.g., tetrahydrofuranyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 366, Compound 499, Compound 500, or a pharmaceutically acceptable salt, solvate, hydrate, tautomer, or stereoisomer thereof.

In some embodiments, for Formula (III), A is bicyclic heteroaryl (e.g., 8-cyano-2-methylimidazo[1,2-a]pyridinyl); B is monocyclic heterocyclyl (e.g., tetrahydropyranyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 367, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., piperidinyl); L¹ and L² are absent; X is C(R³) (e.g., CH); Y is N; Z is N; R² is absent; and R^{7a} and R^{7b} are each independently hydrogen. In some embodiments, the compound of Formula (III), (III-a), and (III-b) is Compound 491, or a pharmaceutically acceptable salt, solvate, hydrate, tautomer, or stereoisomer thereof.

In some embodiments, for Formula (III), A is bicyclic heteroaryl (e.g., 7-fluoro-2-methyl-2H-indazole); B is monocyclic heterocyclyl (e.g., piperidinyl); L¹ and L² are absent; X is C(R³) (e.g., CH); Y is N; Z is N; R² is absent; and R^{7a} and R^{7b} are each independently hydrogen. In some embodiments, the compound of Formula (III), (III-a), and (III-b) is Compound 492, or a pharmaceutically acceptable salt, solvate, hydrate, tautomer, or stereoisomer thereof.

In some embodiments, for Formula (III), A is bicyclic heteroaryl (e.g., 8-cyano-2-methylimidazo[1,2-a]pyridinyl); B is monocyclic heterocyclyl (e.g., piperidinyl); L¹ and L² are absent; X is C(R³) (e.g., CH); Y is N; Z is N; R² is absent; and R^{7a} and R^{7b} are each independently hydrogen. In some embodiments, the compound of Formula (III), (III-a), and (III-b) is Compound 493, or a pharmaceutically acceptable salt, solvate, hydrate, tautomer, or stereoisomer thereof.

In some embodiments, for Formula (III), A is bicyclic heteroaryl (e.g., 6-hydroxy-2,7-dimethyl-2H-indazolyl); B is monocyclic heterocyclyl (e.g., piperidinyl); L¹ and L² are absent; X is C(R³) (e.g., CH); Y is N; Z is N; R² is absent; and R^{7a} and R^{7b} are each independently hydrogen. In some embodiments, the compound of Formula (III), (III-a), and (III-b) is Compound 494, or a pharmaceutically acceptable salt, solvate, hydrate, tautomer, or stereoisomer thereof.

In some embodiments, for Formula (III), A is bicyclic heteroaryl (e.g., 6-fluoro-2,7-dimethyl-2H-indazolyl); B is monocyclic heterocyclyl (e.g., piperidinyl); L¹ and L² are absent; X is C(R³) (e.g., CH); Y is N; Z is N; R² is absent; and R^{7a} and R^{7b} are each independently hydrogen. In some embodiments, the compound of Formula (III), (III-a), and (III-b) is Compound 495, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., piperidinyl); L¹ and L² are absent; X is C(R³) (e.g., CH); Y is N; Z is N; R² is absent; and R^{7a} and R^{7b} are each independently hydrogen. In some embodiments, the compound of Formula (III), (III-a), and (III-b) is Compound 496, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is bicyclic heterocyclyl (e.g., 4-azaspiro[2.5]octanyl); L¹ and L² are absent; X is C(R³) (e.g., CH); Y is N; Z is N; R² is absent; and R^{7a} and R^{7b} are each independently hydrogen. In some embodiments, the compound of Formula (III), (III-a), and (III-b) is Compound 497, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., pyrrolidinyl); L¹ and L² are absent; X is C(R³) (e.g., CH); Y is N; Z is N; R² is absent; and R^{7a} and R^{7b} are each independently hydrogen. In some embodiments, the compound of Formula (III), (III-a), and (III-b) is Compound 498, or a pharmaceutically acceptable salt, solvate, hydrate, tautomer, or stereoisomer thereof.

In some embodiments, for Formula (III), A is bicyclic heteroaryl (e.g., 8-cyano-2-methylimidazo[1,2-b]pyridazyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 501, or a pharmaceutically acceptable salt, solvate, hydrate, tautomer, or stereoisomer thereof.

In some embodiments, for Formula (III), A is bicyclic heteroaryl (e.g., 2-methyl-5,6,7,8-tetrahydroimidazo[1,2-a]pyridinyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 502, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., piperidinyl); L¹ and L² are absent; X and Z are independently C(R³) (e.g., CH); Y is N; R² is absent; and R^{7a} is hydrogen; and R^{7b} is OR (e.g., OH). In some embodiments, the compound of Formula (III), (III-a), and (III-c) is Compound 503, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., piperidinyl); L¹ and L² are absent; X and Z are independently C(R³) (e.g., CH); Y is N; R² is absent; and R^{7a} is hydrogen; and R^{7b} is OR (e.g., OCH₃). In some embodiments, the compound of Formula (III), (III-a), and (III-c) is Compound 504, or a pharmaceutically acceptable salt, solvate, hydrate, tautomer, or stereoisomer thereof.

In some embodiments, for Formula (III), A is bicyclic heteroaryl (e.g., 6-hydroxy-2,7-dimethyl-2H-indazolyl); B is monocyclic heterocyclyl (e.g., piperidinyl); L¹ and L² are absent; X is C(R³) (e.g., CH); Y and Z are independently N; R² is absent; and R^{7a} is hydrogen; and R^{7b} is hydrogen. In some embodiments, the compound of Formula (III), (III-a), and (III-b) is Compound 505, or a pharmaceutically acceptable salt, solvate, hydrate, tautomer, or stereoisomer thereof.

In some embodiments, for Formula (III), A is bicyclic heteroaryl (e.g., 6-methoxy-2,7-dimethyl-2H-indazolyl); B is monocyclic heterocyclyl (e.g., piperidinyl); L¹ and L² are absent; X is C(R³) (e.g., CH); Y and Z are independently N; R² is absent; and R^{7a} is hydrogen; and R^{7b} is hydrogen. In some embodiments, the compound of Formula (III), (III-a), and (III-b) is Compound 506, or a pharmaceutically acceptable salt, solvate, hydrate, tautomer, or stereoisomer thereof.

In some embodiments, for Formula (III), A is bicyclic heteroaryl (e.g., 8-cyano-2-methylimidazo[1,2-a]pyridinyl); B is monocyclic heterocyclyl (e.g., pyrrolidinyl); L¹ and L² are absent; X is C(R³) (e.g., COH); Y is N; Z is C(R³) (e.g., CH); R² is absent; and R^{7a} and R^{7b} are each independently hydrogen. In some embodiments, the compound of Formula (III), (III-a), and (III-c) is Compound 507, or a pharmaceutically acceptable salt, solvate, hydrate, tautomer, or stereoisomer thereof.

In some embodiments, for Formula (III), A is bicyclic heteroaryl (e.g., 2-methyl-4,5,6,7-tetrahydro[1,3]oxazolo[5,4-c]pyridinyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 508, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., piperidinyl); L¹ and L² are absent; X is C(R³) (e.g., CH); Y is N; Z is C(R³) (e.g., CH); R² is absent; R^{7a} is hydrogen; and R^{7b} is fluoro. In some embodiments, the compound of Formula (III), (III-a), and (III-c) is Compound 585, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., piperidinyl); L¹ and L² are absent; X is C(R³) (e.g., CH); Y is N; Z is C(R³) (e.g., CH); R² is absent; R^{7a} is hydrogen; and R^{7b} is fluoro. In some embodiments, the compound of Formula (III), (III-a), and (III-c) is Compound 586, or a pharmaceutically acceptable salt, solvate, hydrate, tautomer, or stereoisomer thereof.

In some embodiments, for Formula (III), A is bicyclic heteroaryl (e.g., 8-fluoro-2-methylimidazo[1,2-a]pyridinyl); B is monocyclic heterocyclyl (e.g., piperidinyl); L¹ and L² are absent; X is C(R³) (e.g., C(OH)); Y is N; Z is C(R³) (e.g., CH); R² is absent; and R^{7a} and R^{7b} are each independently hydrogen. In some embodiments, the compound of Formula (III), (III-a), and (III-c) is Compound 587, or a pharmaceutically acceptable salt, solvate, hydrate, tautomer, or stereoisomer thereof.

In some embodiments, for Formula (III), A is bicyclic heteroaryl (e.g., 8-cyano-2-methylimidazo[1,2-a]pyridinyl); B is monocyclic heterocyclyl (e.g., piperidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 588, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., pyrrolidinyl); L¹ and L² are absent; X is C(R³) (e.g., CF); Y is N; Z is C(R³) (e.g., CH); R² is absent; and R^{7a} and R^{7b} are each independently hydrogen. In some embodiments, the compound of Formula (III), (III-a), and (III-c) is Compound 589, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazyl); B is monocyclic heterocyclyl (e.g., piperidinyl); L¹ and L² are absent; X is C(R³) (e.g., CH); Y is C; Z is C(R³) (e.g., CH); R² is C(R³) (e.g., CH₃); and R^{7a} and R⁷ are each independently hydrogen. In some embodiments, the compound of Formula (III) is Compound 590, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-a]pyrazinyl); B is monocyclic heterocyclyl (e.g., piperidinyl); L¹ and L² are absent; X is C(R³) (e.g., CH); Y is C; Z is C(R³) (e.g., CH); R² is C(R³) (e.g., CH₃); and R^{7a} and R⁷ are each independently hydrogen. In some embodiments, the compound of Formula (III) is Compound 591, or a pharmaceutically acceptable salt, solvate, hydrate, tautomer, or stereoisomer thereof.

In some embodiments, the compound of Formula (IV) is a compound of Formula (IV-a):

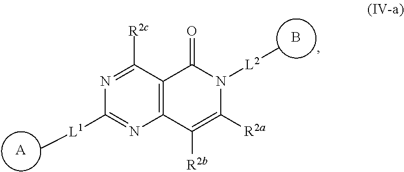

or a pharmaceutically acceptable salt, solvate, hydrate, tautomer, or stereoisomer thereof, wherein A, B, L¹, L², R^{2a}, R^{2b}, R^{2c}, and subvariables thereof are as defined herein.

In some embodiments, the compound of Formula (IV) is a compound of Formula (IV-b):

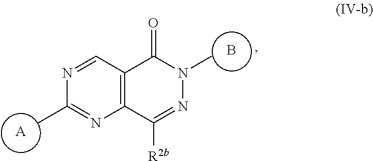

or a pharmaceutically acceptable salt, solvate, hydrate, tautomer, or stereoisomer thereof, wherein A, B, R^{2b}, and subvariables thereof are as defined herein.

In some embodiments, the compound of Formula (IV) is a compound of Formula (IV-c):

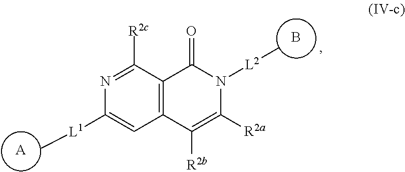

or a pharmaceutically acceptable salt, solvate, hydrate, tautomer, or stereoisomer thereof, wherein A, B, L¹, L², R^{2a}, R^{2b}, R^{2c}, and subvariables thereof are as defined herein.

In some embodiments, the compound of Formula (IV) is a compound of Formula (IV-d):

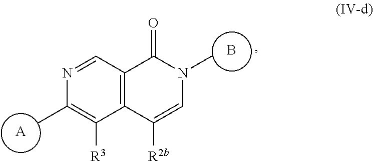

or a pharmaceutically acceptable salt, solvate, hydrate, tautomer, or stereoisomer thereof, wherein A, B, R³, R^{2b}, and subvariables thereof are as defined herein.

In some embodiments, the compound of Formula (IV) is a compound of Formula (I-e):

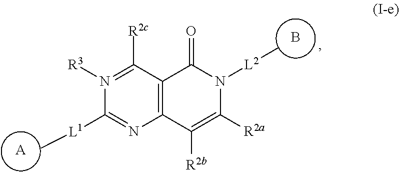

or a pharmaceutically acceptable salt, solvate, hydrate, tautomer, or stereoisomer thereof, wherein A, B, L¹, L², R^{2a}, R^{2b}, R^{2c}, R³, and subvariables thereof are as defined herein.

In some embodiments, the compound of Formula (IV) is a compound of Formula (IV-f):

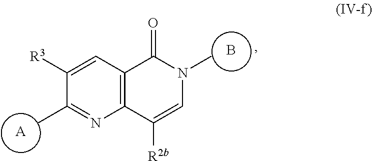

or a pharmaceutically acceptable salt, solvate, hydrate, tautomer, or stereoisomer thereof, wherein A, B, R³, R^{2b}, and subvariables thereof are as defined herein.

In some embodiments, the compound of Formula (IV) is a compound provided in Table 4.

TABLE 4
Exemplary Compounds of Formula (IV)

| Cd | |
|---|---|
| No. | Structure |
| 1121 | 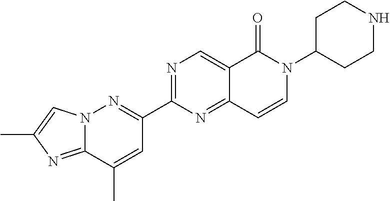 [SMILES: Cc1cn2nc(-c3ncc4c(=O)n(C5CCNCC5)ccc4n3)cc(C)c2n1] |
| 1122 | 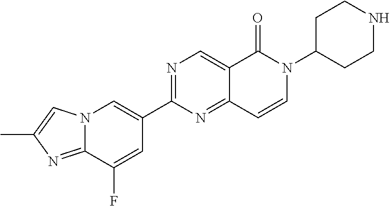 [SMILES: Cc1cn2cc(-c3ncc4c(=O)n(C5CCNCC5)ccc4n3)cc(F)c2n1] |
| 1123 | 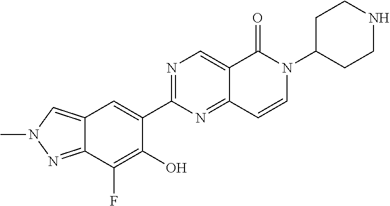 [SMILES: Cn1cc2cc(-c3ncc4c(=O)n(C5CCNCC5)ccc4n3)c(O)c(F)c2n1] |
| 1124 | 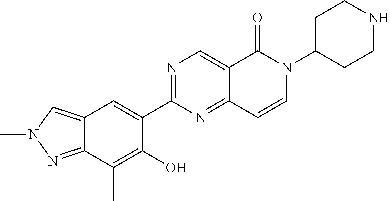 [SMILES: Cc1c(O)c(-c2ncc3c(=O)n(C4CCNCC4)ccc3n2)cc2cn(C)nc12] |
| 1125 | 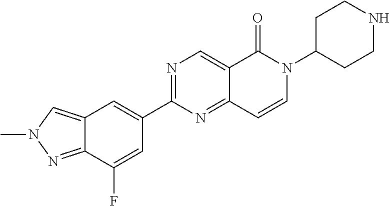 [SMILES: Cn1cc2cc(-c3ncc4c(=O)n(C5CCNCC5)ccc4n3)cc(F)c2n1] |
| 1126 | 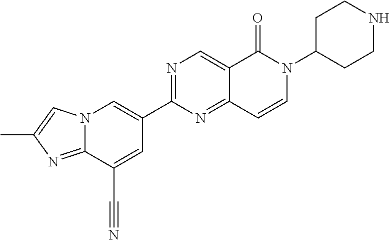 [SMILES: Cc1cn2cc(-c3ncc4c(=O)n(C5CCNCC5)ccc4n3)cc(C#N)c2n1] |
| 1127 | 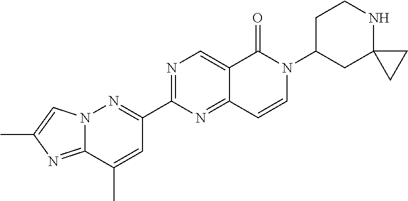 [SMILES: Cc1cn2nc(-c3ncc4c(=O)n(C5CCNC6(CC6)C5)ccc4n3)cc(C)c2n1] |
| 1128 | 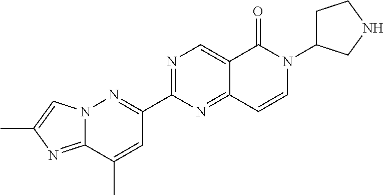 [SMILES: Cc1cn2nc(-c3ncc4c(=O)n(C5CCNC5)ccc4n3)cc(C)c2n1] |
| 1188 | 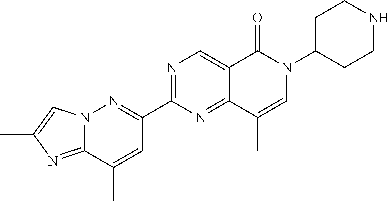 [SMILES: Cc1cn2nc(-c3ncc4c(=O)n(C5CCNCC5)cc(C)c4n3)cc(C)c2n1] |
| 1189 | 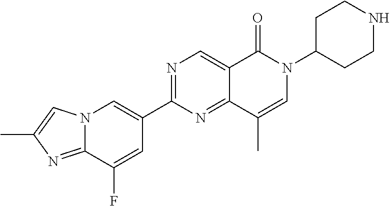 [SMILES: Cc1cn2cc(-c3ncc4c(=O)n(C5CCNCC5)cc(C)c4n3)cc(F)c2n1] |
| 1190 | 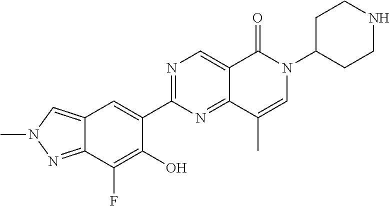 [SMILES: Cc1cn(C2CCNCC2)c(=O)c2cnc(-c3cc4cn(C)nc4c(F)c3O)nc12] |
| 1191 | 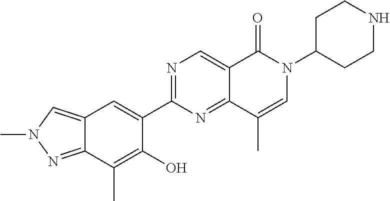 [SMILES: Cc1c(O)c(-c2ncc3c(=O)n(C4CCNCC4)cc(C)c3n2)cc2cn(C)nc12] |
| 1192 | 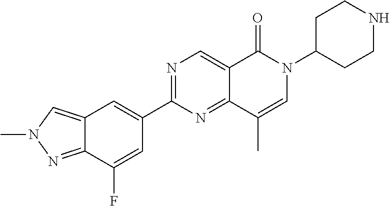 [SMILES: Cc1cn(C2CCNCC2)c(=O)c2cnc(-c3cc(F)c4nn(C)cc4c3)nc12] |
| 1193 | 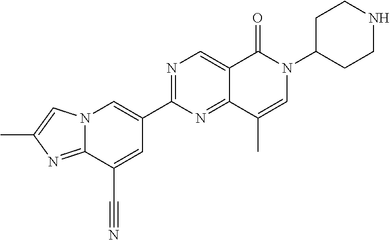 [SMILES: Cc1cn2cc(-c3ncc4c(=O)n(C5CCNCC5)cc(C)c4n3)cc(C#N)c2n1] |
| 1194 | 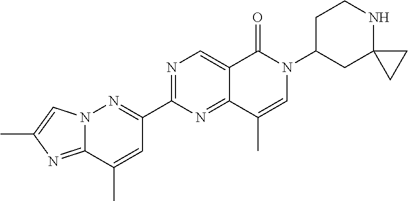 [SMILES: Cc1cn2nc(-c3ncc4c(=O)n(C5CCNC6(CC6)C5)cc(C)c4n3)cc(C)c2n1] |
| 1195 | 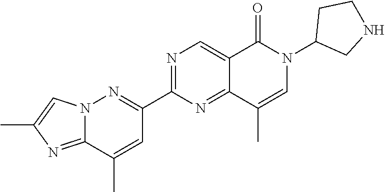 [SMILES: Cc1cn2nc(-c3ncc4c(=O)n(C5CCNC5)cc(C)c4n3)cc(C)c2n1] |
| 1196 | 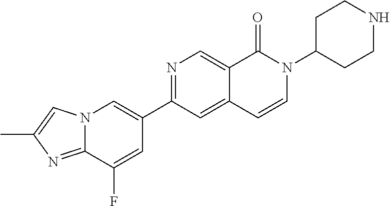 [SMILES: Cc1cn2cc(-c3cc4ccn(C5CCNCC5)c(=O)c4cn3)cc(F)c2n1] |
| 1197 | 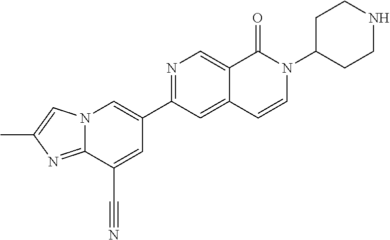 [SMILES: Cc1cn2cc(-c3cc4ccn(C5CCNCC5)c(=O)c4cn3)cc(C#N)c2n1] |
| 1198 | 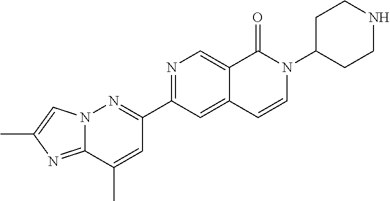 [SMILES: Cc1cn2nc(-c3cc4ccn(C5CCNCC5)c(=O)c4cn3)cc(C)c2n1] |
| 1199 | 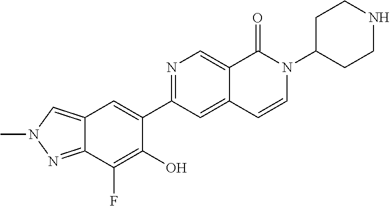 [SMILES: Cn1cc2cc(-c3cc4ccn(C5CCNCC5)c(=O)c4cn3)c(O)c(F)c2n1] |
| 1200 | 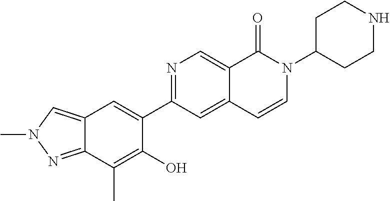 [SMILES: Cc1c(O)c(-c2cc3ccn(C4CCNCC4)c(=O)c3cn2)cc2cn(C)nc12] |
| 1201 | 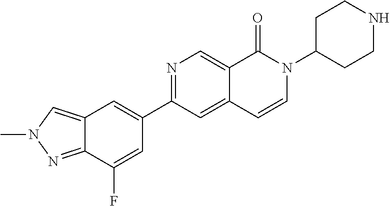 [SMILES: Cn1cc2cc(-c3cc4ccn(C5CCNCC5)c(=O)c4cn3)cc(F)c2n1] |
| 1202 | 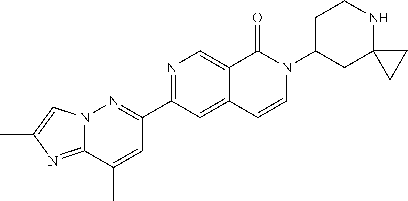 [SMILES: Cc1cn2nc(-c3cc4ccn(C5CCNC6(CC6)C5)c(=O)c4cn3)cc(C)c2n1] |
| 1203 | 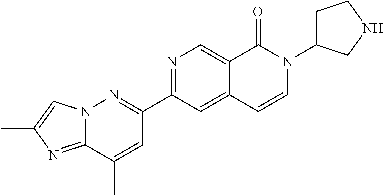 [SMILES: Cc1cn2nc(-c3cc4ccn(C5CCNC5)c(=O)c4cn3)cc(C)c2n1] |
| 1204 | 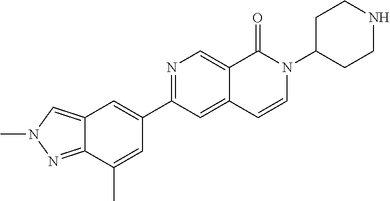 [SMILES: Cc1cc(-c2cc3ccn(C4CCNCC4)c(=O)c3cn2)cc2cn(C)nc12] |
| 1205 | 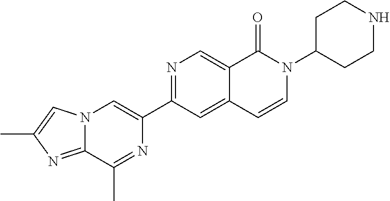 [SMILES: Cc1cn2cc(-c3cc4ccn(C5CCNCC5)c(=O)c4cn3)nc(C)c2n1] |
| 1206 | 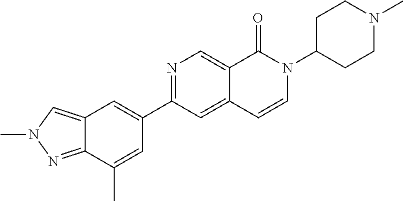 |
| 1207 | 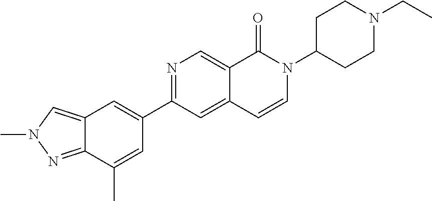 [SMILES: CCN1CCC(n2ccc3cc(-c4cc(C)c5nn(C)cc5c4)ncc3c2=O)CC1] |
| 1208 | 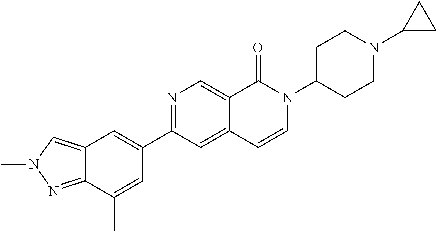 [SMILES: Cc1cc(-c2cc3ccn(C4CCN(C5CC5)CC4)c(=O)c3cn2)cc2cn(C)nc12] |
| 1209 | 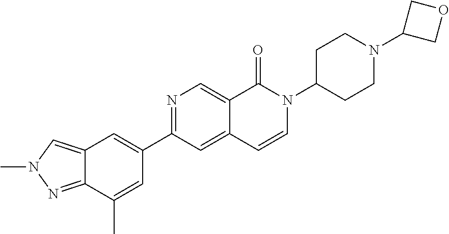 [SMILES: Cc1cc(-c2cc3ccn(C4CCN(C5COC5)CC4)c(=O)c3cn2)cc2cn(C)nc12] |
| 1210 | 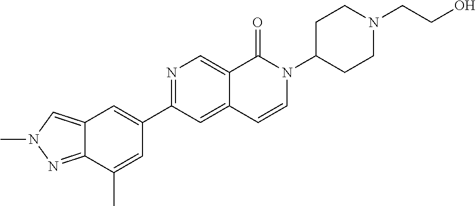 [SMILES: Cc1cc(-c2cc3ccn(C4CCN(CCO)CC4)c(=O)c3cn2)cc2cn(C)nc12] |
| 1211 | 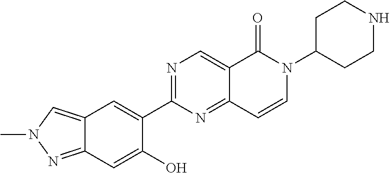 [SMILES: Cn1cc2cc(-c3ncc4c(=O)n(C5CCNCC5)ccc4n3)c(O)cc2n1] |
| 1212 | 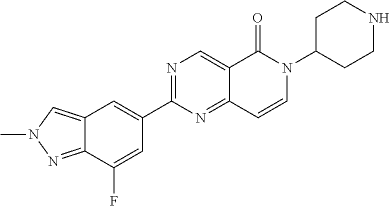 [SMILES: Cn1cc2cc(-c3ncc4c(=O)n(C5CCNCC5)ccc4n3)cc(F)c2n1] |
| 1213 | 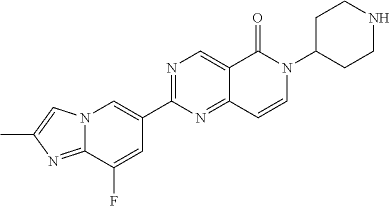 [SMILES: Cc1cn2cc(-c3ncc4c(=O)n(C5CCNCC5)ccc4n3)cc(F)c2n1] |
| 1214 | 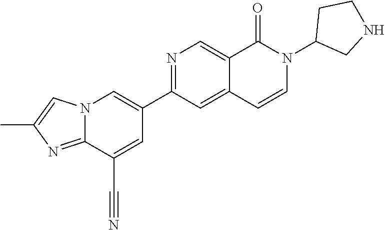 [SMILES: Cc1cn2cc(-c3cc4ccn(C5CCNC5)c(=O)c4cn3)cc(C#N)c2n1] |
| 1215 | 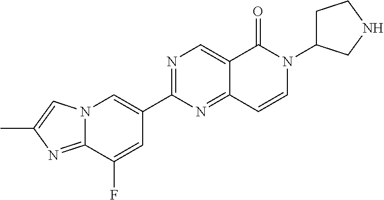 [SMILES: Cc1cn2cc(-c3ncc4c(=O)n(C5CCNC5)ccc4n3)cc(F)c2n1] |
| 1218 | 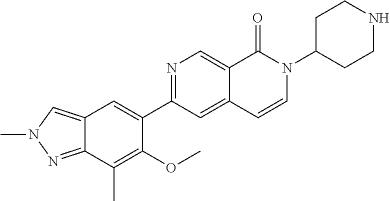 [SMILES: COc1c(-c2cc3ccn(C4CCNCC4)c(=O)c3cn2)cc2cn(C)nc2c1C] |
| 1219 | 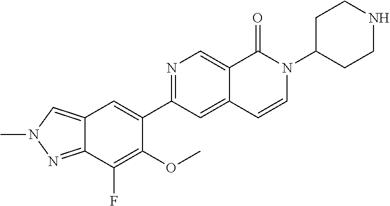 [SMILES: COc1c(-c2cc3ccn(C4CCNCC4)c(=O)c3cn2)cc2cn(C)nc2c1F] |
| 1220 | 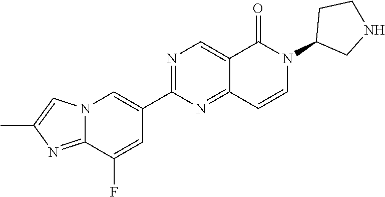 [SMILES: Cc1cn2cc(-c3ncc4c(=O)n([C@H]5CCNC5)ccc4n3)cc(F)c2n1] |
| 1221 | 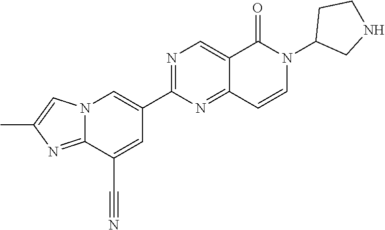 [SMILES: Cc1cn2cc(-c3ncc4c(=O)n(C5CCNC5)ccc4n3)cc(C#N)c2n1] |
| 1222 | 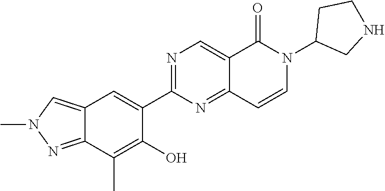 [SMILES: Cc1c(O)c(-c2ncc3c(=O)n(C4CCNC4)ccc3n2)cc2cn(C)nc12] |
| 1223 | 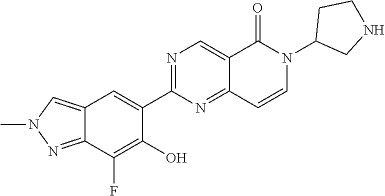 [SMILES: Cn1cc2cc(-c3ncc4c(=O)n(C5CCNC5)ccc4n3)c(O)c(F)c2n1] |
| 1224 | 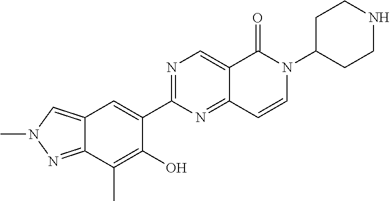 [SMILES: Cc1c(O)c(-c2ncc3c(=O)n(C4CCNCC4)ccc3n2)cc2cn(C)nc12] |
| 1225 | 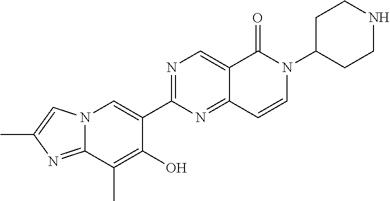 [SMILES: Cc1cn2cc(-c3ncc4c(=O)n(C5CCNCC5)ccc4n3)c(O)c(C)c2n1] |
| 1226 | 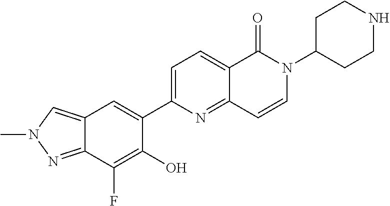 [SMILES: Cn1cc2cc(-c3ccc4c(=O)n(C5CCNCC5)ccc4n3)c(O)c(F)c2n1] |
| 1227 | 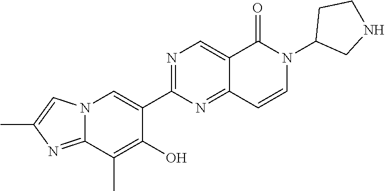 [SMILES: Cc1cn2cc(-c3ncc4c(=O)n(C5CCNC5)ccc4n3)c(O)c(C)c2n1] |
| 1228 | 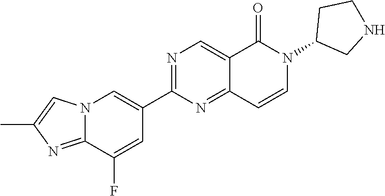 [SMILES: Cc1cn2cc(-c3ncc4c(=O)n([C@@H]5CCNC5)ccc4n3)cc(F)c2n1] |
| 1229 | 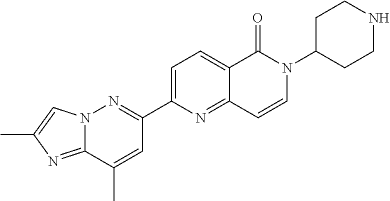 [SMILES: Cc1cn2nc(-c3ccc4c(=O)n(C5CCNCC5)ccc4n3)cc(C)c2n1] |
| 1230 | 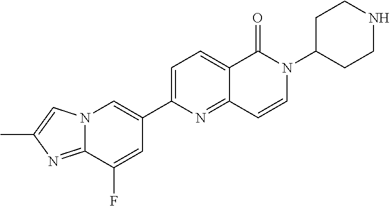 [SMILES: Cc1cn2cc(-c3ccc4c(=O)n(C5CCNCC5)ccc4n3)cc(F)c2n1] |
| 1231 | 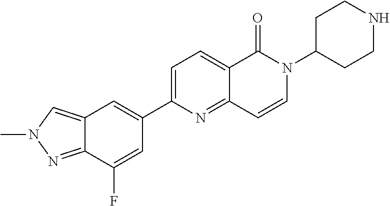 [SMILES: Cn1cc2cc(-c3ccc4c(=O)n(C5CCNCC5)ccc4n3)cc(F)c2n1] |
| 1232 | 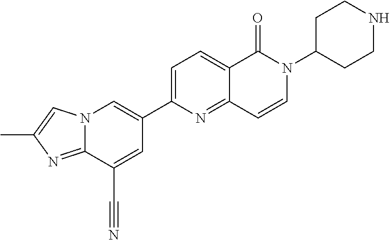 [SMILES: Cc1cn2cc(-c3ccc4c(=O)n(C5CCNCC5)ccc4n3)cc(C#N)c2n1] |
| 1233 | 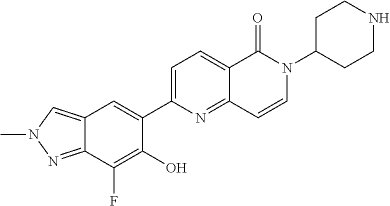 [SMILES: Cn1cc2cc(-c3ccc4c(=O)n(C5CCNCC5)ccc4n3)c(O)c(F)c2n1] |
| 1234 | 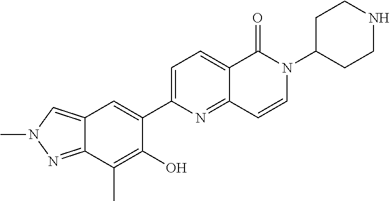 [SMILES: Cc1c(O)c(-c2ccc3c(=O)n(C4CCNCC4)ccc3n2)cc2cn(C)nc12] |
| 1235 | 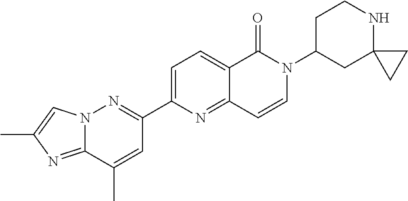 [SMILES: Cc1cn2nc(-c3ccc4c(=O)n(C5CCNC6(CC6)C5)ccc4n3)cc(C)c2n1] |
| 1236 | 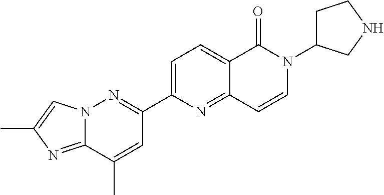 [SMILES: Cc1cn2nc(-c3ccc4c(=O)n(C5CCNC5)ccc4n3)cc(C)c2n1] |
| 1237 | 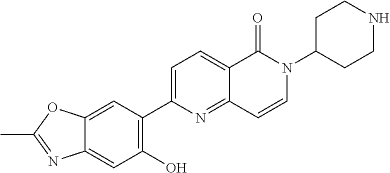 [SMILES: Cc1nc2cc(O)c(-c3ccc4c(=O)n(C5CCNCC5)ccc4n3)cc2o1] |
| 1238 | 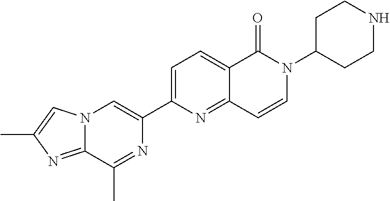 [SMILES: Cc1cn2cc(-c3ccc4c(=O)n(C5CCNCC5)ccc4n3)nc(C)c2n1] |
| 1239 | 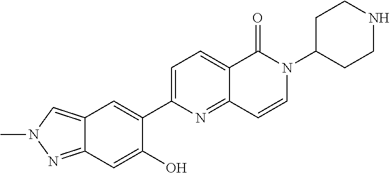 [SMILES: Cn1cc2cc(-c3ccc4c(=O)n(C5CCNCC5)ccc4n3)c(O)cc2n1] |
| 1240 | 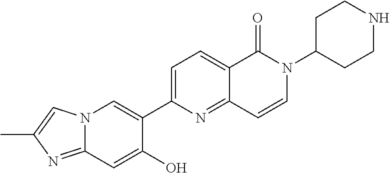 [SMILES: Cc1cn2cc(-c3ccc4c(=O)n(C5CCNCC5)ccc4n3)c(O)cc2n1] |
| 1241 | 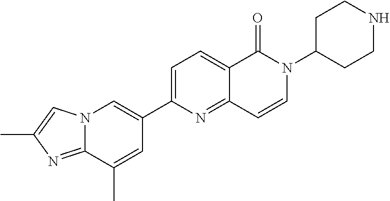 [SMILES: Cc1cn2cc(-c3ccc4c(=O)n(C5CCNCC5)ccc4n3)cc(C)c2n1] |
| 1242 | 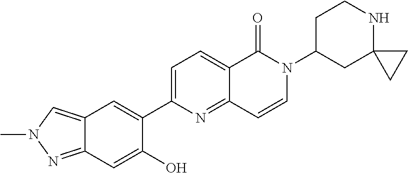 [SMILES: Cn1cc2cc(-c3ccc4c(=O)n(C5CCNC6(CC6)C5)ccc4n3)c(O)cc2n1] |
| 1243 | 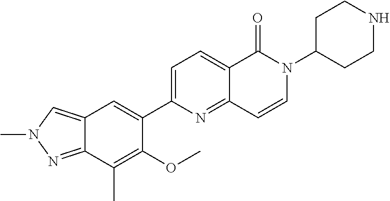 [SMILES: COc1c(-c2ccc3c(=O)n(C4CCNCC4)ccc3n2)cc2cn(C)nc2c1C] |
| 1244 | 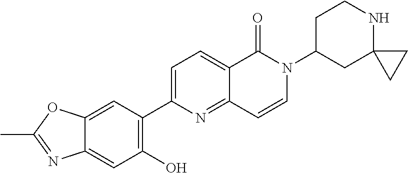 [SMILES: Cc1nc2cc(O)c(-c3ccc4c(=O)n(C5CCNC6(CC6)C5)ccc4n3)cc2o1] |
| 1245 | 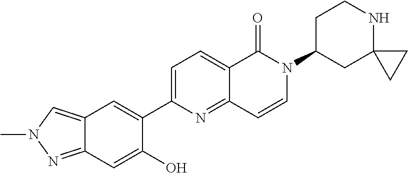 [SMILES: Cn1cc2cc(-c3ccc4c(=O)n([C@H]5CCNC6(CC6)C5)ccc4n3)c(O)cc2n1] |
| 1246 | 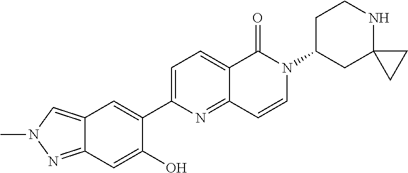 [SMILES: Cn1cc2cc(-c3ccc4c(=O)n([C@@H]5CCNC6(CC6)C5)ccc4n3)c(O)cc2n1] |
| 1283 | 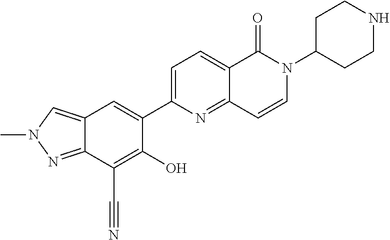 [SMILES: Cn1cc2cc(-c3ccc4c(=O)n(C5CCNCC5)ccc4n3)c(O)c(C#N)c2n1] |
| 1284 | 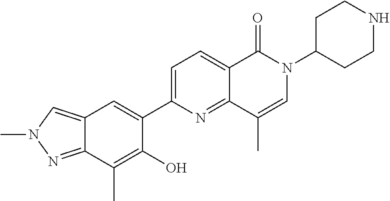 [SMILES: Cc1c(O)c(-c2ccc3c(=O)n(C4CCNCC4)cc(C)c3n2)cc2cn(C)nc12] |
| 1285 | 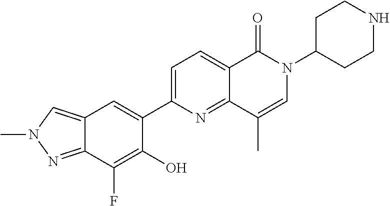 [SMILES: Cc1cn(C2CCNCC2)c(=O)c2ccc(-c3cc4cn(C)nc4c(F)c3O)nc12] |
| 1296 | 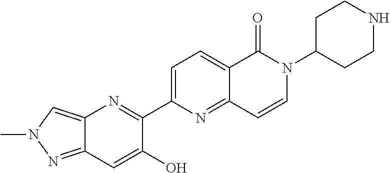 [SMILES: Cn1cc2nc(-c3ccc4c(=O)n(C5CCNCC5)ccc4n3)c(O)cc2n1] |
| 1297 | 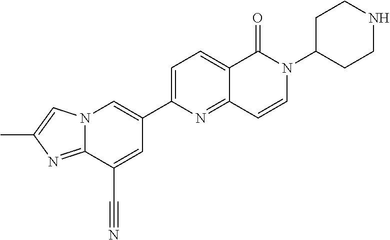 [SMILES: Cc1cn2cc(-c3ccc4c(=O)n(C5CCNCC5)ccc4n3)cc(C#N)c2n1] |
| 1299 | 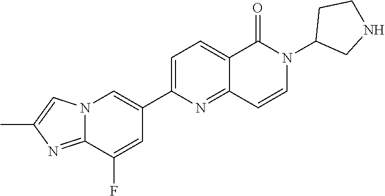 [SMILES: Cc1cn2cc(-c3ccc4c(=O)n(C5CCNC5)ccc4n3)cc(F)c2n1] |
| 1300 | 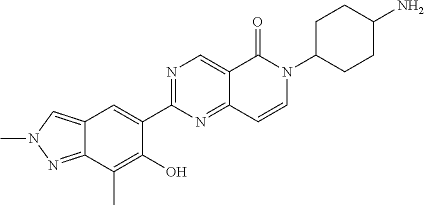 [SMILES: Cc1c(O)c(-c2ncc3c(=O)n(C4CCC(N)CC4)ccc3n2)cc2cn(C)nc12] |
| 1301 | 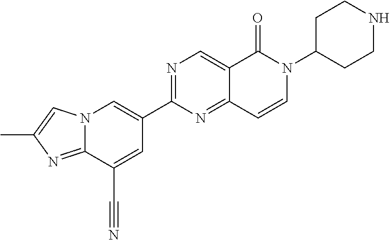 [SMILES: Cc1cn2cc(-c3ncc4c(=O)n(C5CCNCC5)ccc4n3)cc(C#N)c2n1] |
| 1302 | 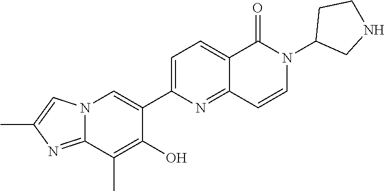 [SMILES: Cc1cn2cc(-c3ccc4c(=O)n(C5CCNC5)ccc4n3)c(O)c(C)c2n1] |
| 1303 | 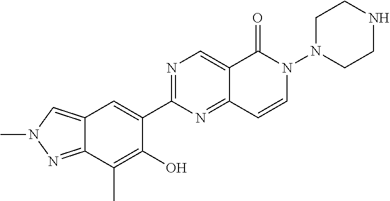 [SMILES: Cc1c(O)c(-c2ncc3c(=O)n(N4CCNCC4)ccc3n2)cc2cn(C)nc12] |
| 1304 | 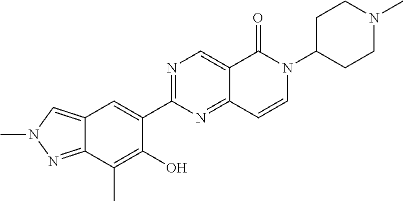 |
| 1305 | 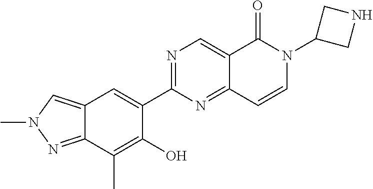 [SMILES: Cc1c(O)c(-c2ncc3c(=O)n(C4CNC4)ccc3n2)cc2cn(C)nc12] |
| 1320 | 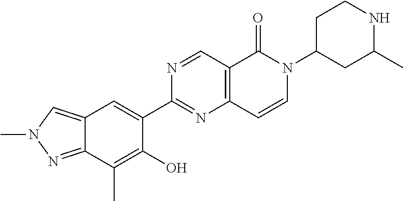 |
| 1321 | 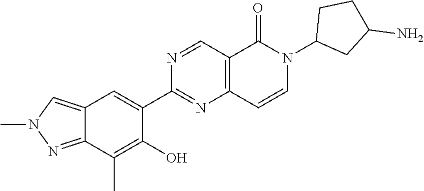 [SMILES: Cc1c(O)c(-c2ncc3c(=O)n(C4CCC(N)C4)ccc3n2)cc2cn(C)nc12] |
| 1322 | 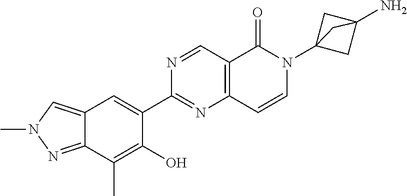 [SMILES: Cc1c(O)c(-c2ncc3c(=O)n(C45CC(N)(C4)C5)ccc3n2)cc2cn(C)nc12] |
| 1323 | 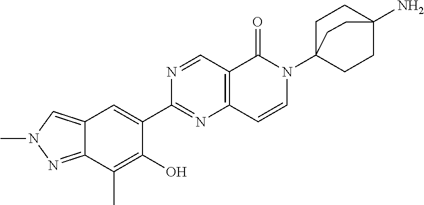 [SMILES: Cc1c(O)c(-c2ncc3c(=O)n(C45CCC(N)(CC4)CC5)ccc3n2)cc2cn(C)nc12] |
| 1324 | 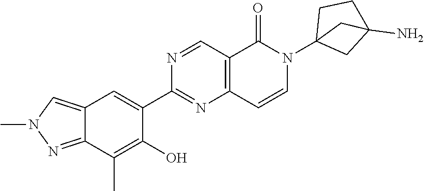 [SMILES: Cc1c(O)c(-c2ncc3c(=O)n(C45CCC(N)(C4)C5)ccc3n2)cc2cn(C)nc12] |
| 1325 | 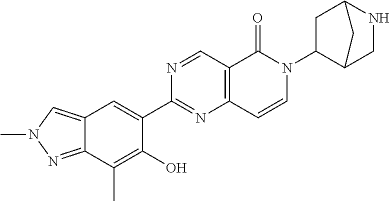 [SMILES: Cc1c(O)c(-c2ncc3c(=O)n(C4CC5CC4CN5)ccc3n2)cc2cn(C)nc12] |
| 1326 | 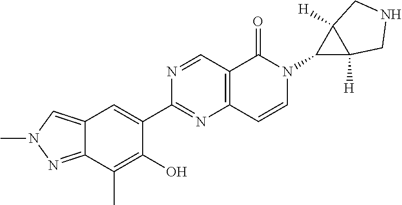 [SMILES: Cc1c(O)c(-c2ncc3c(=O)n([C@H]4[C@@H]5CNC[C@@H]54)ccc3n2)cc2cn(C)nc12] |
| 1327 | 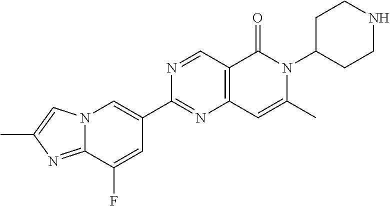 [SMILES: Cc1cn2cc(-c3ncc4c(=O)n(C5CCNCC5)c(C)cc4n3)cc(F)c2n1] |
| 1328 | 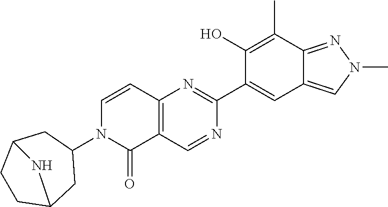 [SMILES: Cc1c(O)c(-c2ncc3c(=O)n(C4CC5CCC(C4)N5)ccc3n2)cc2cn(C)nc12] |
| 1329 | 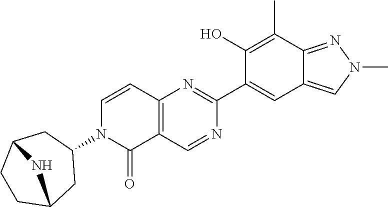 [SMILES: Cc1c(O)c(-c2ncc3c(=O)n([C@H]4C[C@H]5CC[C@@H](C4)N5)ccc3n2)cc2cn(C)nc12] |
| 1330 | 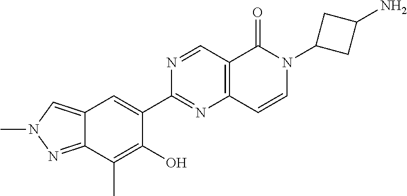 [SMILES: Cc1c(O)c(-c2ncc3c(=O)n(C4CC(N)C4)ccc3n2)cc2cn(C)nc12] |
| 1331 | 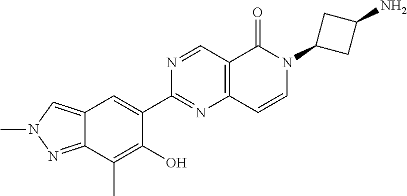 [SMILES: Cc1c(O)c(-c2ncc3c(=O)n([C@H]4C[C@@H](N)C4)ccc3n2)cc2cn(C)nc12] |
| 1332 | 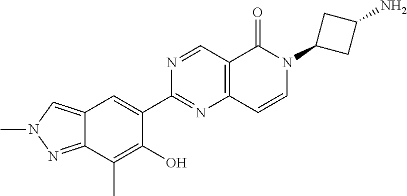 [SMILES: Cc1c(O)c(-c2ncc3c(=O)n([C@H]4C[C@H](N)C4)ccc3n2)cc2cn(C)nc12] |
| 1333 | 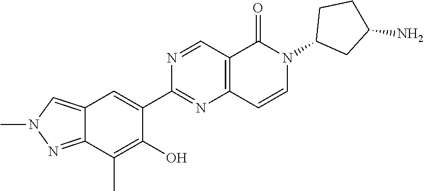 [SMILES: Cc1c(O)c(-c2ncc3c(=O)n([C@@H]4CC[C@H](N)C4)ccc3n2)cc2cn(C)nc12] |
| 1334 | 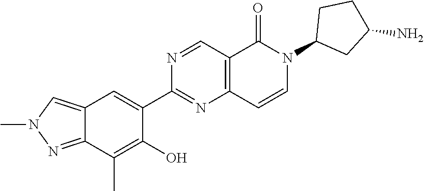 [SMILES: Cc1c(O)c(-c2ncc3c(=O)n([C@H]4CC[C@H](N)C4)ccc3n2)cc2cn(C)nc12] |
| 1335 | 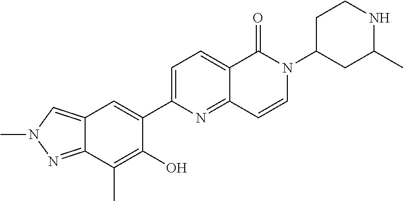 |
| 1336 | 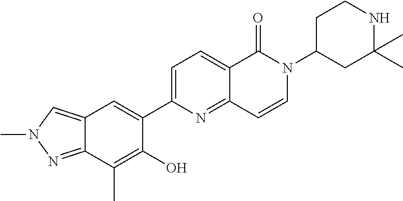 |
| 1337 | 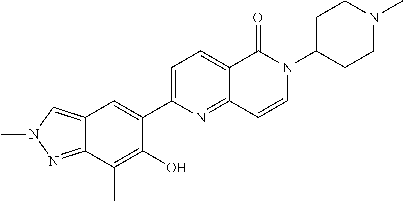 |
| 1338 | 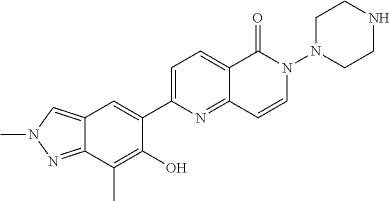 [SMILES: Cc1c(O)c(-c2ccc3c(=O)n(N4CCNCC4)ccc3n2)cc2cn(C)nc12] |
| 1339 | 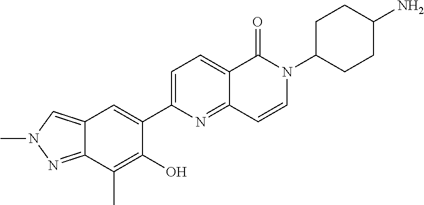 [SMILES: Cc1c(O)c(-c2ccc3c(=O)n(C4CCC(N)CC4)ccc3n2)cc2cn(C)nc12] |
| 1340 | 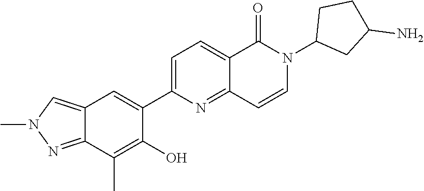 [SMILES: Cc1c(O)c(-c2ccc3c(=O)n(C4CCC(N)C4)ccc3n2)cc2cn(C)nc12] |
| 1341 | 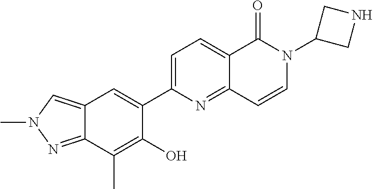 [SMILES: Cc1c(O)c(-c2ccc3c(=O)n(C4CNC4)ccc3n2)cc2cn(C)nc12] |
| 1342 | 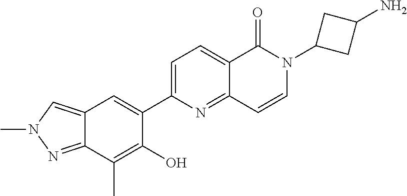 [SMILES: Cc1c(O)c(-c2ccc3c(=O)n(C4CC(N)C4)ccc3n2)cc2cn(C)nc12] |
| 1343 | 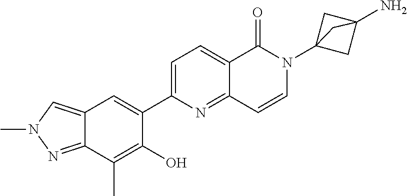 [SMILES: Cc1c(O)c(-c2ccc3c(=O)n(C45CC(N)(C4)C5)ccc3n2)cc2cn(C)nc12] |
| 1344 | 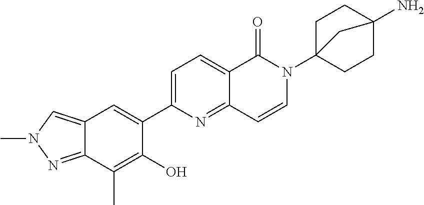 [SMILES: Cc1c(O)c(-c2ccc3c(=O)n(C45CCC(N)(CC4)C5)ccc3n2)cc2cn(C)nc12] |
| 1345 | 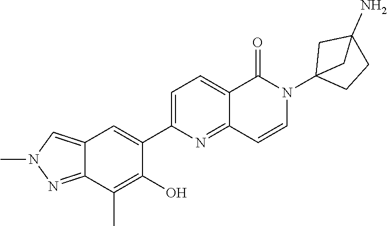 [SMILES: Cc1c(O)c(-c2ccc3c(=O)n(C45CCC(N)(C4)C5)ccc3n2)cc2cn(C)nc12] |
| 1346 | 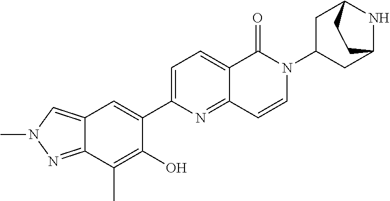 [SMILES: Cc1c(O)c(-c2ccc3c(=O)n(C4C[C@H]5CC[C@@H](C4)N5)ccc3n2)cc2cn(C)nc12] |
| 1347 | 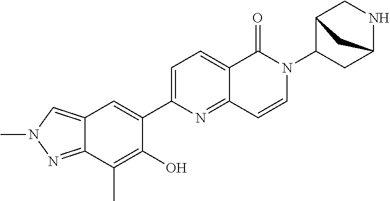 [SMILES: Cc1c(O)c(-c2ccc3c(=O)n(C4C[C@@H]5C[C@H]4CN5)ccc3n2)cc2cn(C)nc12] |
| 1348 | 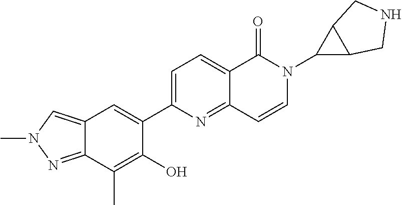 [SMILES: Cc1c(O)c(-c2ccc3c(=O)n(C4C5CNCC54)ccc3n2)cc2cn(C)nc12] |
| 1349 | 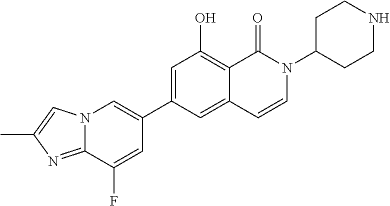 [SMILES: Cc1cn2cc(-c3cc(O)c4c(=O)n(C5CCNCC5)ccc4c3)cc(F)c2n1] |
| 1350 | 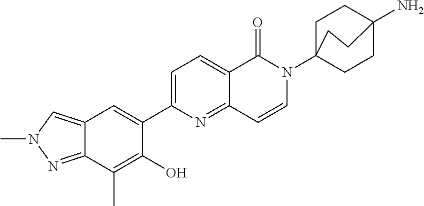 [SMILES: Cc1c(O)c(-c2ccc3c(=O)n(C45CCC(N)(CC4)CC5)ccc3n2)cc2cn(C)nc12] |
| 1351 | 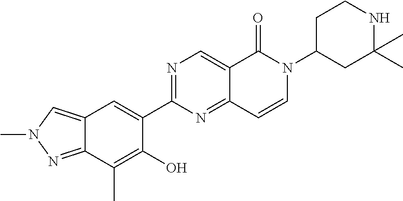 |
| 1352 | 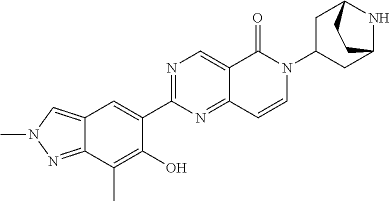 [SMILES: Cc1c(O)c(-c2ncc3c(=O)n(C4C[C@H]5CC[C@@H](C4)N5)ccc3n2)cc2cn(C)nc12] |
| 1353 | 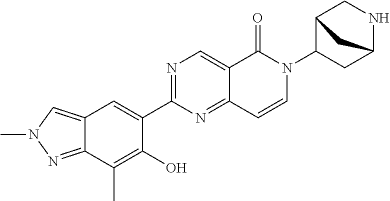 [SMILES: Cc1c(O)c(-c2ncc3c(=O)n(C4C[C@@H]5C[C@H]4CN5)ccc3n2)cc2cn(C)nc12] |
| 1354 | 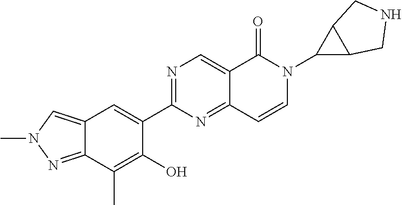 [SMILES: Cc1c(O)c(-c2ncc3c(=O)n(C4C5CNCC54)ccc3n2)cc2cn(C)nc12] |
| 1355 | 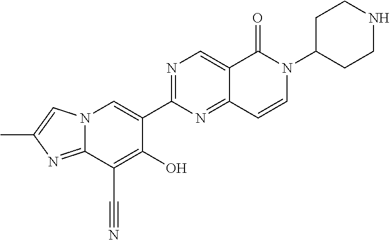 [SMILES: Cc1cn2cc(-c3ncc4c(=O)n(C5CCNCC5)ccc4n3)c(O)c(C#N)c2n1] |
| 1356 | 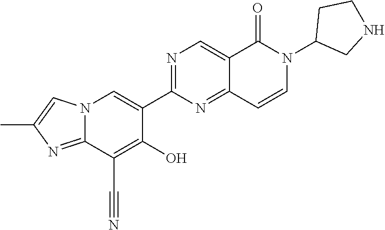 [SMILES: Cc1cn2cc(-c3ncc4c(=O)n(C5CCNC5)ccc4n3)c(O)c(C#N)c2n1] |
| 1357 | 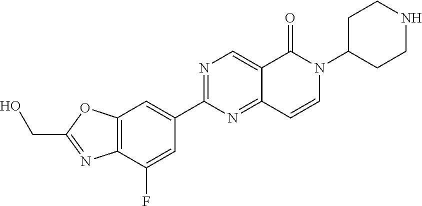 [SMILES: O=c1c2cnc(-c3cc(F)c4nc(CO)oc4c3)nc2ccn1C1CCNCC1] |
| 1358 | 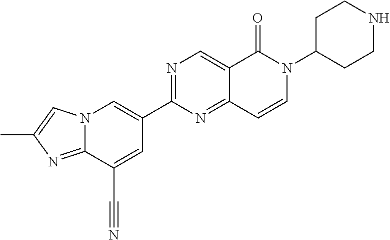 [SMILES: Cc1cn2cc(-c3ncc4c(=O)n(C5CCNCC5)ccc4n3)cc(C#N)c2n1] |
| 1359 | 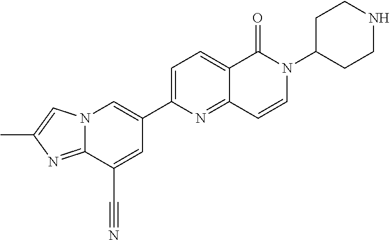 [SMILES: Cc1cn2cc(-c3ccc4c(=O)n(C5CCNCC5)ccc4n3)cc(C#N)c2n1] |
| 1360 | 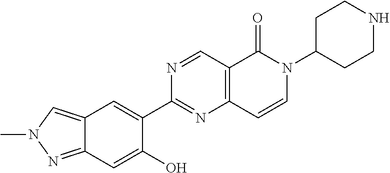 [SMILES: Cn1cc2cc(-c3ncc4c(=O)n(C5CCNCC5)ccc4n3)c(O)cc2n1] |
| 1361 | 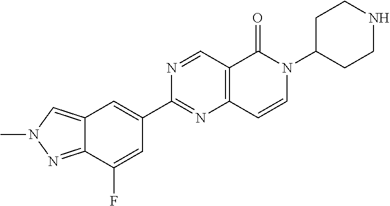 [SMILES: Cn1cc2cc(-c3ncc4c(=O)n(C5CCNCC5)ccc4n3)cc(F)c2n1] |
| 1362 | 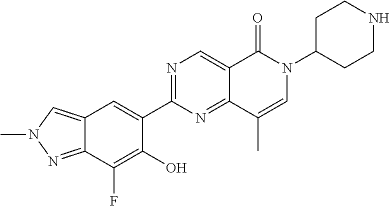 [SMILES: Cc1cn(C2CCNCC2)c(=O)c2cnc(-c3cc4cn(C)nc4c(F)c3O)nc12] |
| 1363 | 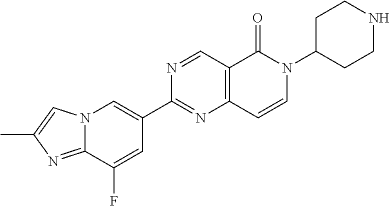 [SMILES: Cc1cn2cc(-c3ncc4c(=O)n(C5CCNCC5)ccc4n3)cc(F)c2n1] |
| 1364 | 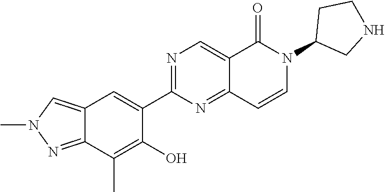 [SMILES: Cc1c(O)c(-c2ncc3c(=O)n([C@H]4CCNC4)ccc3n2)cc2cn(C)nc12] |
| 1365 | 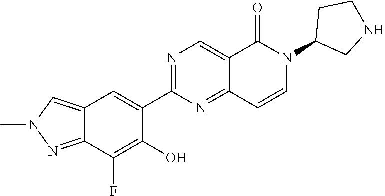 [SMILES: Cn1cc2cc(-c3ncc4c(=O)n([C@H]5CCNC5)ccc4n3)c(O)c(F)c2n1] |
| 1366 | 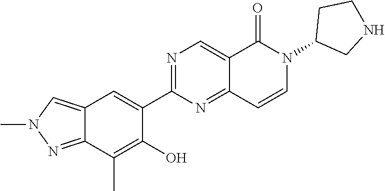 [SMILES: Cc1c(O)c(-c2ncc3c(=O)n([C@@H]4CCNC4)ccc3n2)cc2cn(C)nc12] |
| 1367 | 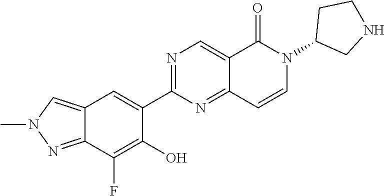 [SMILES: Cn1cc2cc(-c3ncc4c(=O)n([C@@H]5CCNC5)ccc4n3)c(O)c(F)c2n1] |
| 1368 | 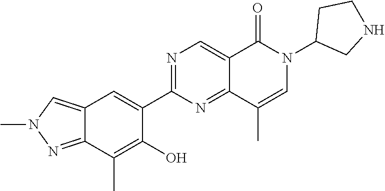 [SMILES: Cc1c(O)c(-c2ncc3c(=O)n(C4CCNC4)cc(C)c3n2)cc2cn(C)nc12] |
| 1369 | 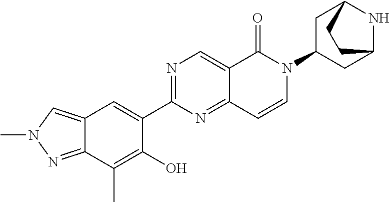 [SMILES: Cc1c(O)c(-c2ncc3c(=O)n([C@H]4C[C@H]5CC[C@@H](C4)N5)ccc3n2)cc2cn(C)nc12] |
| 1370 | 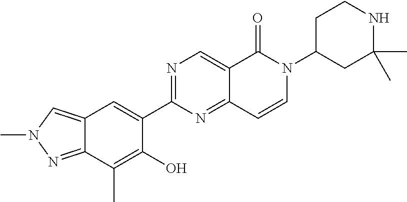 |
| 1371 | 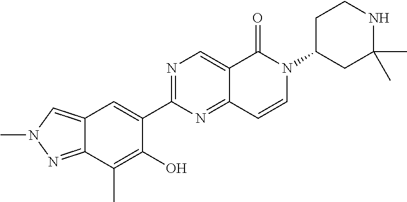 |
| 1372 | 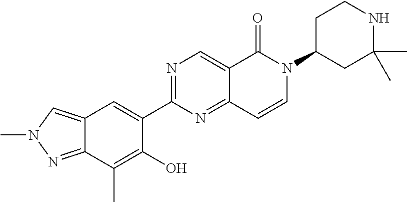 |
| 1373 | 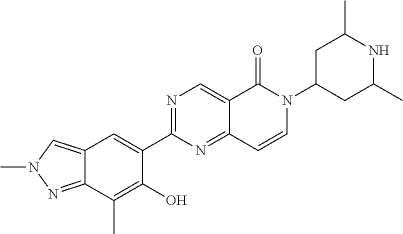 |
| 1374 | 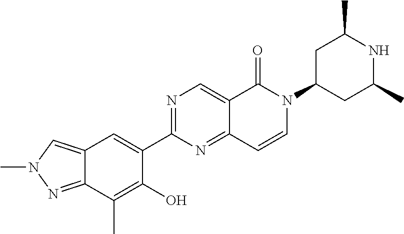 |
| 1375 | 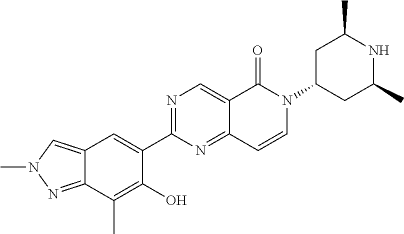 |
| 1376 | 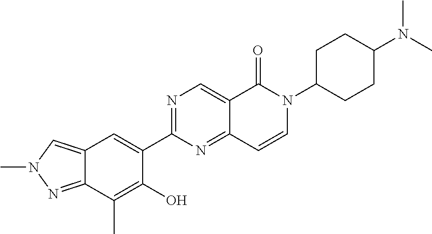 |
| 1377 | 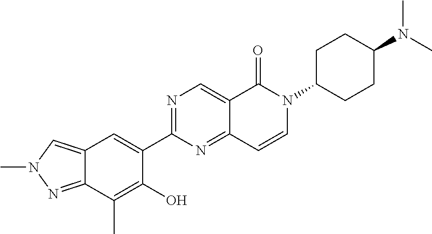 |
| 1378 | 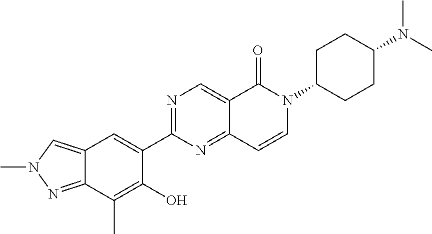 |
| 1379 | 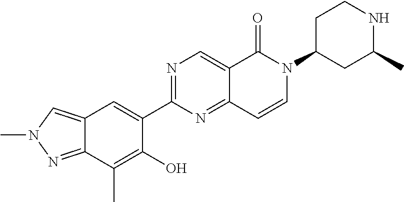 |
| 1380 | 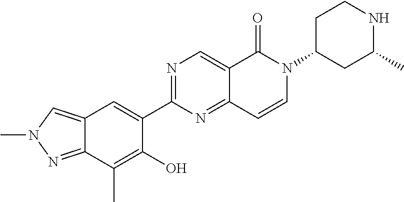 |
| 1381 | 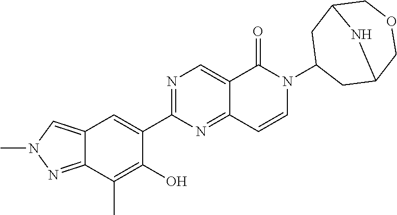 [SMILES: Cc1c(O)c(-c2ncc3c(=O)n(C4CC5COCC(C4)N5)ccc3n2)cc2cn(C)nc12] |
| 1382 | 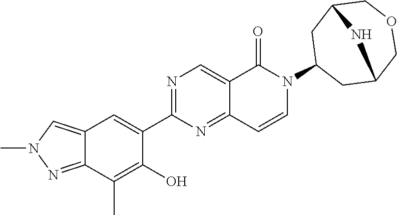 [SMILES: Cc1c(O)c(-c2ncc3c(=O)n([C@@H]4C[C@H]5COC[C@@H](C4)N5)ccc3n2)cc2cn(C)nc12] |
| 1383 | 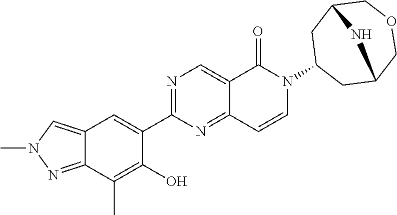 [SMILES: Cc1c(O)c(-c2ncc3c(=O)n([C@H]4C[C@H]5COC[C@@H](C4)N5)ccc3n2)cc2cn(C)nc12] |
| 1384 | 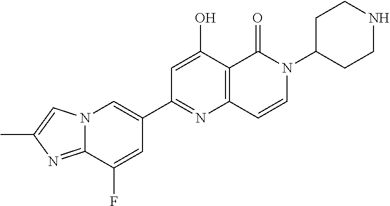 [SMILES: Cc1cn2cc(-c3cc(O)c4c(=O)n(C5CCNCC5)ccc4n3)cc(F)c2n1] |
| 1385 | 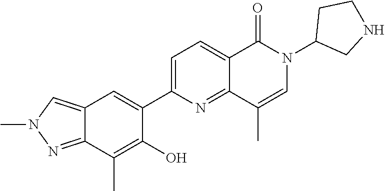 [SMILES: Cc1c(O)c(-c2ccc3c(=O)n(C4CCNC4)cc(C)c3n2)cc2cn(C)nc12] |
| 1386 | 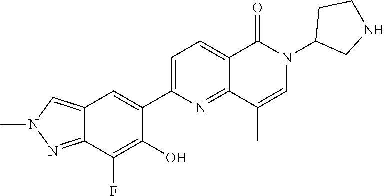 [SMILES: Cc1cn(C2CCNC2)c(=O)c2ccc(-c3cc4cn(C)nc4c(F)c3O)nc12] |
| 1387 | 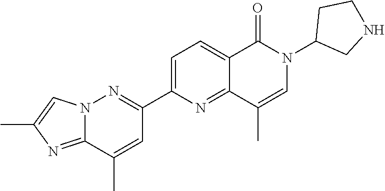 [SMILES: Cc1cn2nc(-c3ccc4c(=O)n(C5CCNC5)cc(C)c4n3)cc(C)c2n1] |
| 1388 | 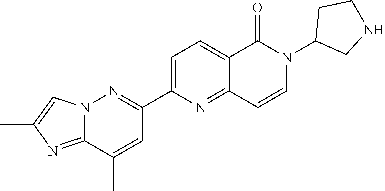 [SMILES: Cc1cn2nc(-c3ccc4c(=O)n(C5CCNC5)ccc4n3)cc(C)c2n1] |
| 1389 | 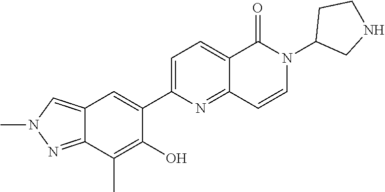 [SMILES: Cc1c(O)c(-c2ccc3c(=O)n(C4CCNC4)ccc3n2)cc2cn(C)nc12] |
| 1390 | 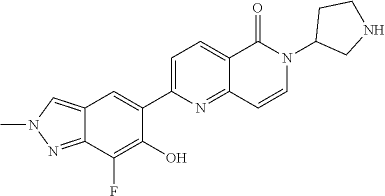 [SMILES: Cn1cc2cc(-c3ccc4c(=O)n(C5CCNC5)ccc4n3)c(O)c(F)c2n1] |
| 1391 | 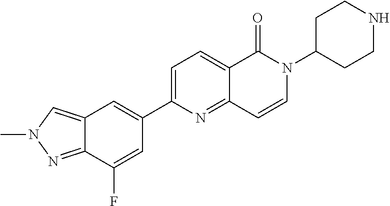 [SMILES: Cn1cc2cc(-c3ccc4c(=O)n(C5CCNCC5)ccc4n3)cc(F)c2n1] |
| 1392 | 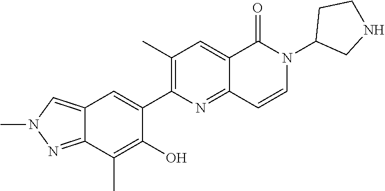 [SMILES: Cc1cc2c(=O)n(C3CCNC3)ccc2nc1-c1cc2cn(C)nc2c(C)c1O] |
| 1393 | 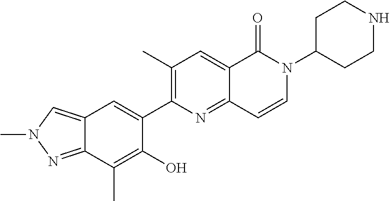 [SMILES: Cc1cc2c(=O)n(C3CCNCC3)ccc2nc1-c1cc2cn(C)nc2c(C)c1O] |
| 1394 | 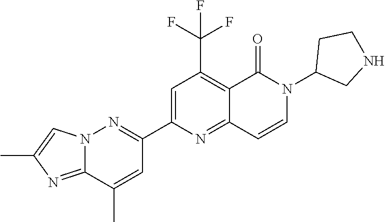 [SMILES: Cc1cn2nc(-c3cc(C(F)(F)F)c4c(=O)n(C5CCNC5)ccc4n3)cc(C)c2n1] |
| 1395 | 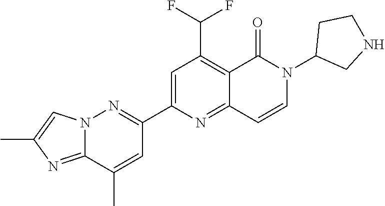 [SMILES: Cc1cn2nc(-c3cc(C(F)F)c4c(=O)n(C5CCNC5)ccc4n3)cc(C)c2n1] |
| 1396 | 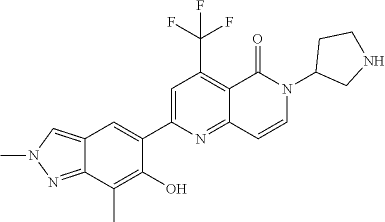 [SMILES: Cc1c(O)c(-c2cc(C(F)(F)F)c3c(=O)n(C4CCNC4)ccc3n2)cc2cn(C)nc12] |
| 1397 | 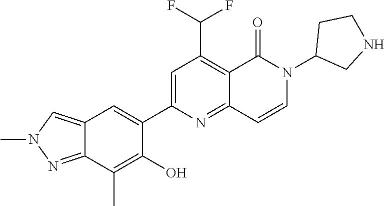 [SMILES: Cc1c(O)c(-c2cc(C(F)F)c3c(=O)n(C4CCNC4)ccc3n2)cc2cn(C)nc12] |
| 1398 | 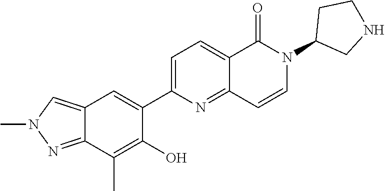 [SMILES: Cc1c(O)c(-c2ccc3c(=O)n([C@H]4CCNC4)ccc3n2)cc2cn(C)nc12] |
| 1399 | 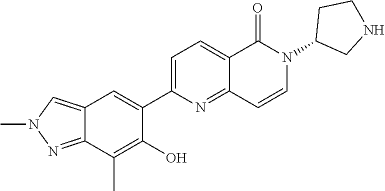 [SMILES: Cc1c(O)c(-c2ccc3c(=O)n([C@@H]4CCNC4)ccc3n2)cc2cn(C)nc12] |
| 1400 | 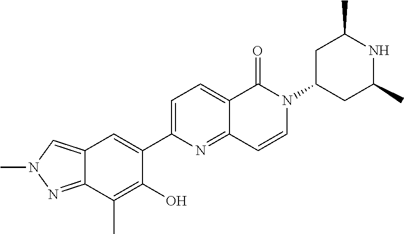 |
| 1401 | 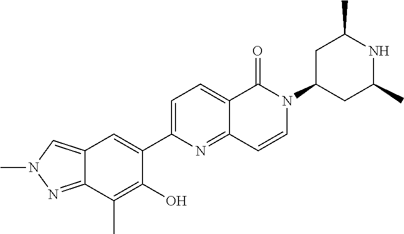 |
| 1402 | 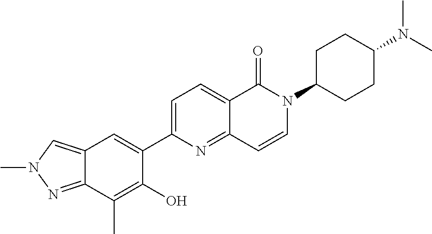 |
| 1403 | 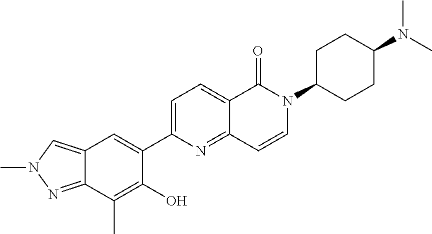 |
| 1404 | 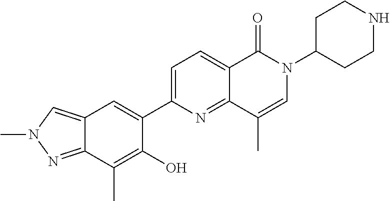 [SMILES: Cc1c(O)c(-c2ccc3c(=O)n(C4CCNCC4)cc(C)c3n2)cc2cn(C)nc12] |
| 1405 | 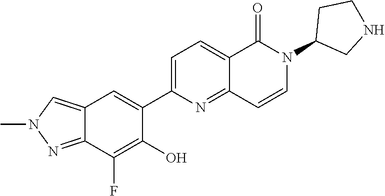 [SMILES: Cn1cc2cc(-c3ccc4c(=O)n([C@H]5CCNC5)ccc4n3)c(O)c(F)c2n1] |
| 1406 | 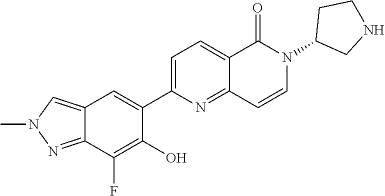 [SMILES: Cn1cc2cc(-c3ccc4c(=O)n([C@@H]5CCNC5)ccc4n3)c(O)c(F)c2n1] |
| 1407 | 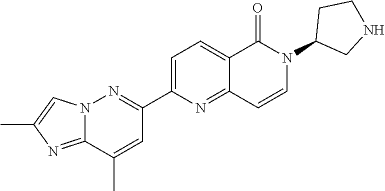 [SMILES: Cc1cn2nc(-c3ccc4c(=O)n([C@H]5CCNC5)ccc4n3)cc(C)c2n1] |
| 1408 | 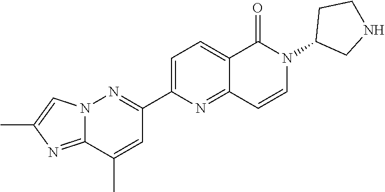 [SMILES: Cc1cn2nc(-c3ccc4c(=O)n([C@@H]5CCNC5)ccc4n3)cc(C)c2n1] |
| 1409 | 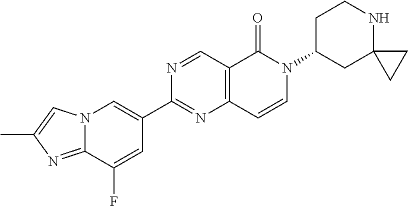 [SMILES: Cc1cn2cc(-c3ncc4c(=O)n([C@@H]5CCNC6(CC6)C5)ccc4n3)cc(F)c2n1] |
| 1410 | 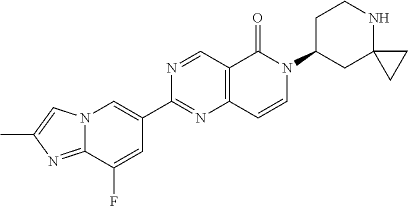 [SMILES: Cc1cn2cc(-c3ncc4c(=O)n([C@H]5CCNC6(CC6)C5)ccc4n3)cc(F)c2n1] |
| 1428 | 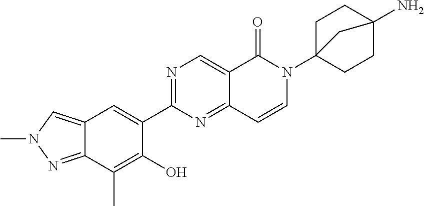 [SMILES: Cc1c(O)c(-c2ncc3c(=O)n(C45CCC(N)(CC4)C5)ccc3n2)cc2cn(C)nc12] |
| 1429 | 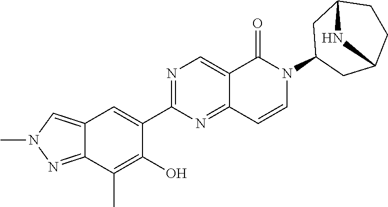 [SMILES: Cc1c(O)c(-c2ncc3c(=O)n([C@@H]4C[C@H]5CC[C@@H](C4)N5)ccc3n2)cc2cn(C)nc12] |
| 1430 | 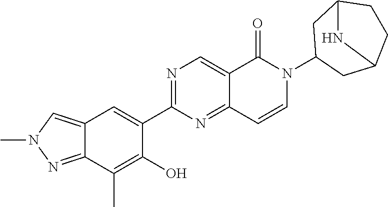 [SMILES: Cc1c(O)c(-c2ncc3c(=O)n(C4CC5CCC(C4)N5)ccc3n2)cc2cn(C)nc12] |
| 1431 | 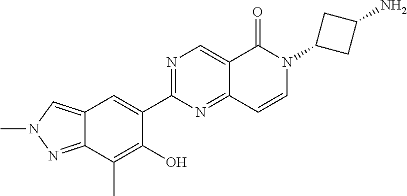 [SMILES: Cc1c(O)c(-c2ncc3c(=O)n([C@H]4C[C@@H](N)C4)ccc3n2)cc2cn(C)nc12] |
| 1432 | 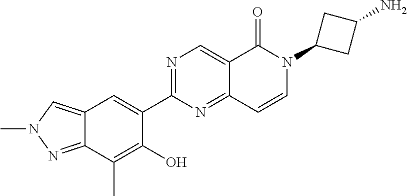 [SMILES: Cc1c(O)c(-c2ncc3c(=O)n([C@H]4C[C@H](N)C4)ccc3n2)cc2cn(C)nc12] |
| 1433 | 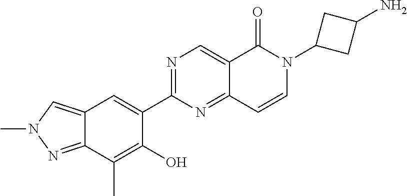 [SMILES: Cc1c(O)c(-c2ncc3c(=O)n(C4CC(N)C4)ccc3n2)cc2cn(C)nc12] |
| 1434 | 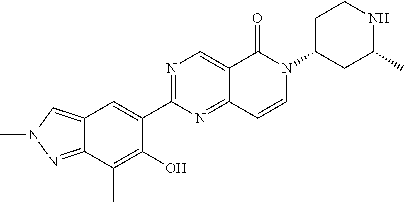 |
| 1435 | 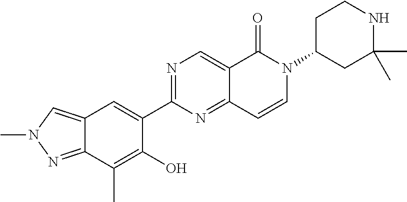 |
| 1436 | 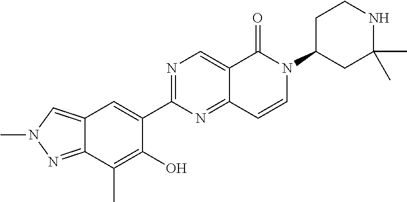 |
| 1437 | 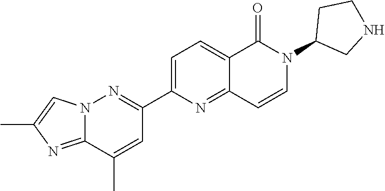 [SMILES: Cc1cn2nc(-c3ccc4c(=O)n([C@H]5CCNC5)ccc4n3)cc(C)c2n1] |
| 1438 | 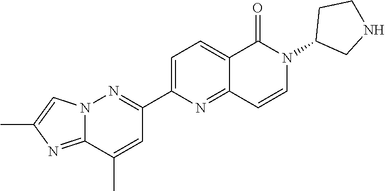 [SMILES: Cc1cn2nc(-c3ccc4c(=O)n([C@@H]5CCNC5)ccc4n3)cc(C)c2n1] |
| 1439 | 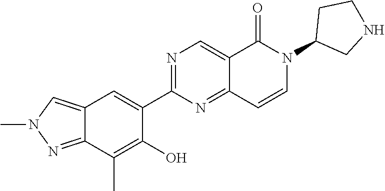 [SMILES: Cc1c(O)c(-c2ncc3c(=O)n([C@H]4CCNC4)ccc3n2)cc2cn(C)nc12] |
| 1440 | 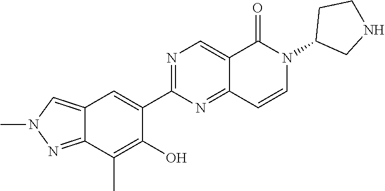 [SMILES: Cc1c(O)c(-c2ncc3c(=O)n([C@@H]4CCNC4)ccc3n2)cc2cn(C)nc12] |
| 1441 | 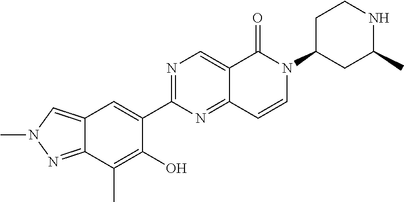 |
| 1442 | 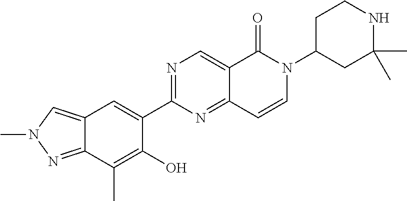 |
| 1443 | 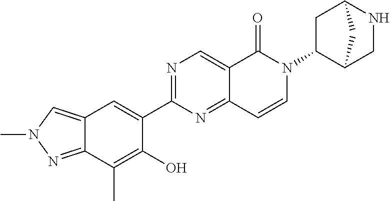 [SMILES: Cc1c(O)c(-c2ncc3c(=O)n([C@@H]4C[C@@H]5C[C@H]4CN5)ccc3n2)cc2cn(C)nc12] |
| 1444 | 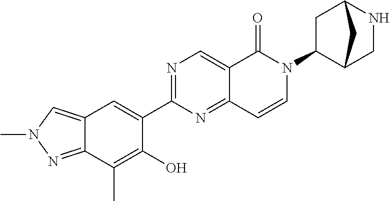 [SMILES: Cc1c(O)c(-c2ncc3c(=O)n([C@H]4C[C@H]5C[C@@H]4CN5)ccc3n2)cc2cn(C)nc12] |
| 1445 | 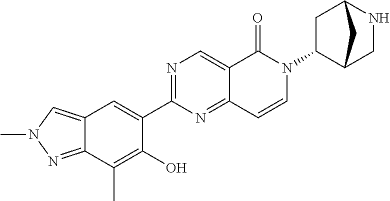 [SMILES: Cc1c(O)c(-c2ncc3c(=O)n([C@@H]4C[C@H]5C[C@@H]4CN5)ccc3n2)cc2cn(C)nc12] |
| 1446 | 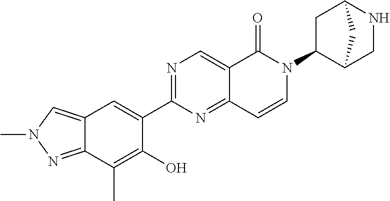 [SMILES: Cc1c(O)c(-c2ncc3c(=O)n([C@H]4C[C@@H]5C[C@H]4CN5)ccc3n2)cc2cn(C)nc12] |
| 1447 | 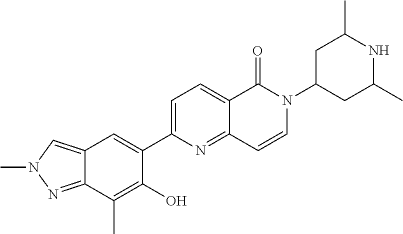 |
| 1448 | 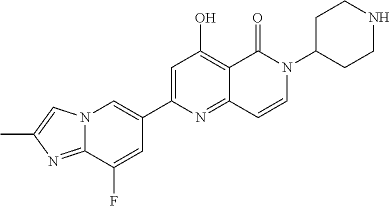 [SMILES: Cc1cn2cc(-c3cc(O)c4c(=O)n(C5CCNCC5)ccc4n3)cc(F)c2n1] |
| 1449 | 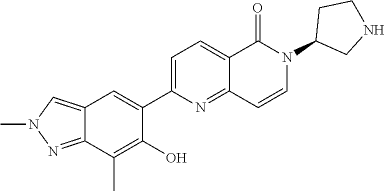 [SMILES: Cc1c(O)c(-c2ccc3c(=O)n([C@H]4CCNC4)ccc3n2)cc2cn(C)nc12] |
| 1450 | 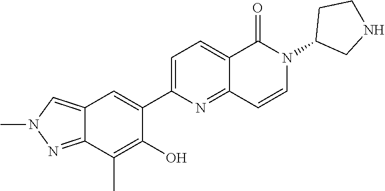 [SMILES: Cc1c(O)c(-c2ccc3c(=O)n([C@@H]4CCNC4)ccc3n2)cc2cn(C)nc12] |
| 1451 | 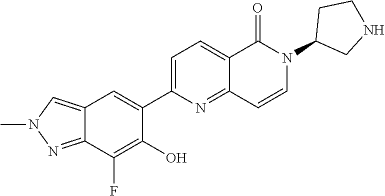 [SMILES: Cn1cc2cc(-c3ccc4c(=O)n([C@H]5CCNC5)ccc4n3)c(O)c(F)c2n1] |
| 1452 | 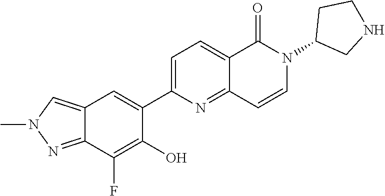 [SMILES: Cn1cc2cc(-c3ccc4c(=O)n([C@@H]5CCNC5)ccc4n3)c(O)c(F)c2n1] |
| 1453 | 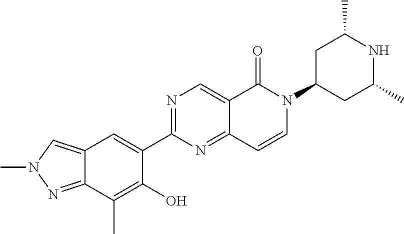 |
| 1454 | 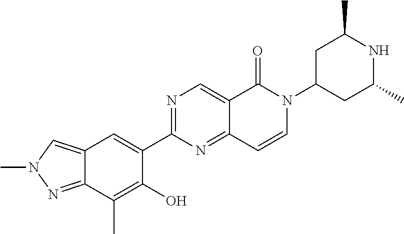 |
| 1455 | 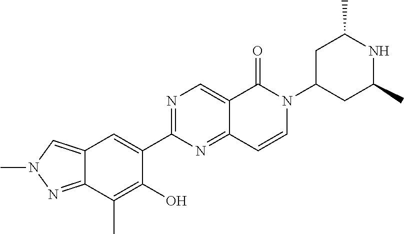 |
| 1456 | 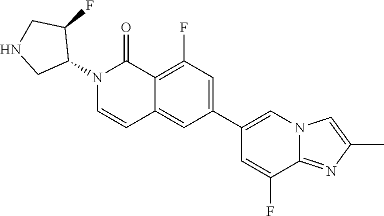 [SMILES: Cc1cn2cc(-c3cc(F)c4c(=O)n([C@@H]5CNC[C@H]5F)ccc4c3)cc(F)c2n1] |
| 1457 | 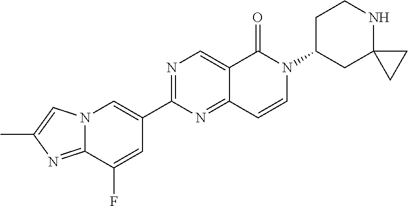 [SMILES: Cc1cn2cc(-c3ncc4c(=O)n([C@@H]5CCNC6(CC6)C5)ccc4n3)cc(F)c2n1] |
| 1458 | 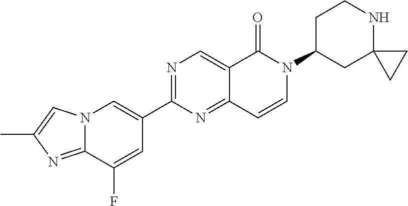 [SMILES: Cc1cn2cc(-c3ncc4c(=O)n([C@H]5CCNC6(CC6)C5)ccc4n3)cc(F)c2n1] |
| 1459 | 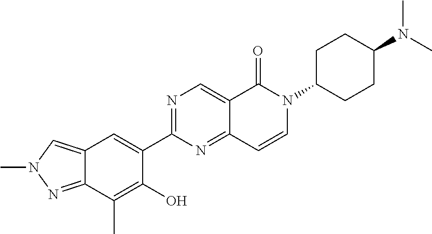 |
| 1460 | 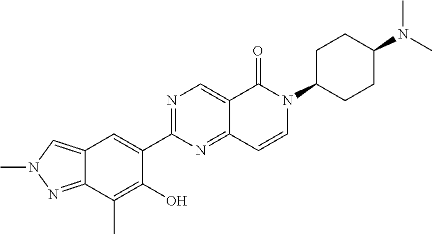 |
| 1461 | 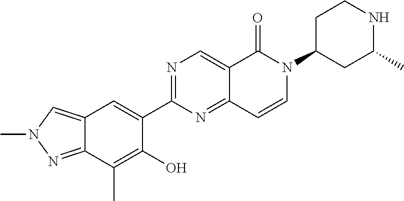 |
| 1462 | 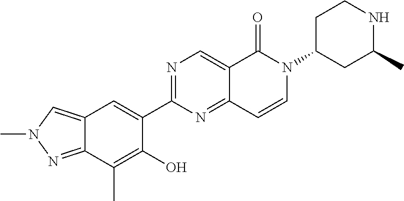 |
| 1463 | 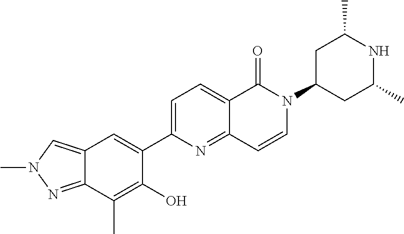 |
| 1464 | 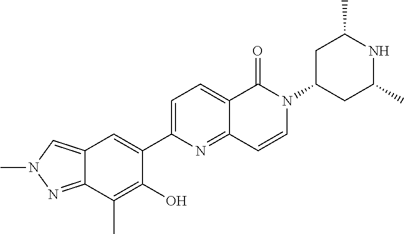 |
| 1465 | 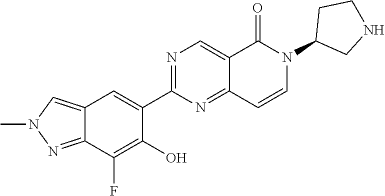 [SMILES: Cn1cc2cc(-c3ncc4c(=O)n([C@H]5CCNC5)ccc4n3)c(O)c(F)c2n1] |
| 1466 | 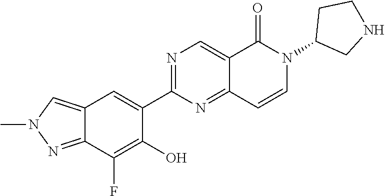 [SMILES: Cn1cc2cc(-c3ncc4c(=O)n([C@@H]5CCNC5)ccc4n3)c(O)c(F)c2n1] |
| 1467 | 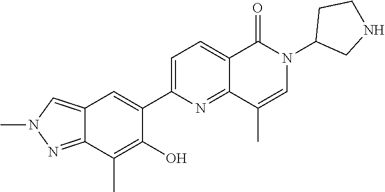 [SMILES: Cc1c(O)c(-c2ccc3c(=O)n(C4CCNC4)cc(C)c3n2)cc2cn(C)nc12] |
| 1468 | 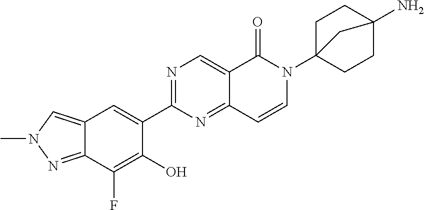 [SMILES: Cn1cc2cc(-c3ncc4c(=O)n(C56CCC(N)(CC5)C6)ccc4n3)c(O)c(F)c2n1] |
| 1469 | 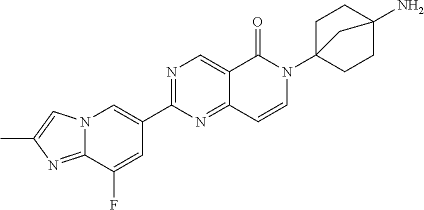 [SMILES: Cc1cn2cc(-c3ncc4c(=O)n(C56CCC(N)(CC5)C6)ccc4n3)cc(F)c2n1] |
| 1470 | 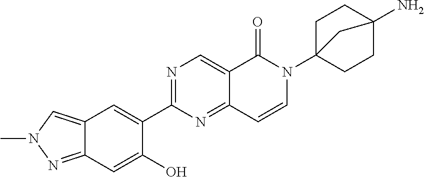 [SMILES: Cn1cc2cc(-c3ncc4c(=O)n(C56CCC(N)(CC5)C6)ccc4n3)c(O)cc2n1] |
| 1471 | 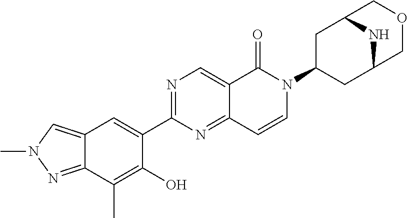 [SMILES: Cc1c(O)c(-c2ncc3c(=O)n([C@@H]4C[C@H]5COC[C@@H](C4)N5)ccc3n2)cc2cn(C)nc12] |
| 1472 | 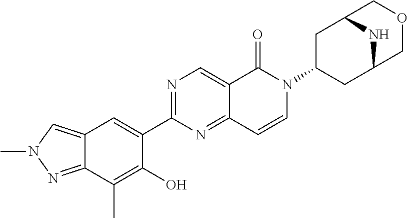 [SMILES: Cc1c(O)c(-c2ncc3c(=O)n([C@H]4C[C@H]5COC[C@@H](C4)N5)ccc3n2)cc2cn(C)nc12] |
| 1473 | 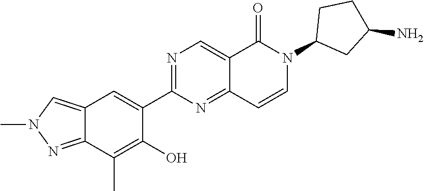 [SMILES: Cc1c(O)c(-c2ncc3c(=O)n([C@H]4CC[C@@H](N)C4)ccc3n2)cc2cn(C)nc12] |
| 1474 | 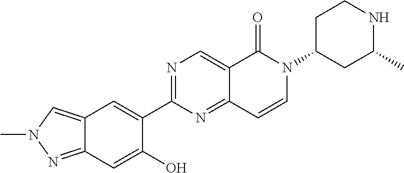 |
| 1475 | 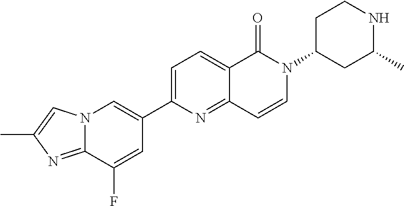 |
| 1476 | 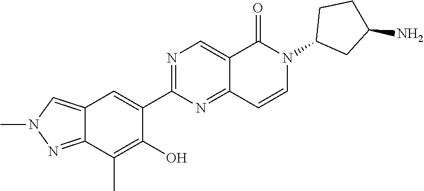 [SMILES: Cc1c(O)c(-c2ncc3c(=O)n([C@@H]4CC[C@@H](N)C4)ccc3n2)cc2cn(C)nc12] |
| 1477 | 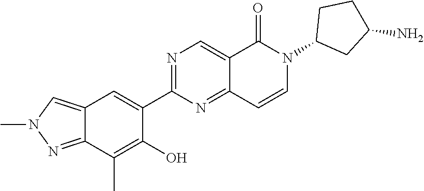 [SMILES: Cc1c(O)c(-c2ncc3c(=O)n([C@@H]4CC[C@H](N)C4)ccc3n2)cc2cn(C)nc12] |
| 1478 | 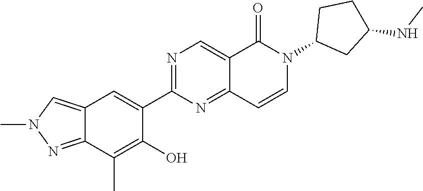 |
| 1479 | 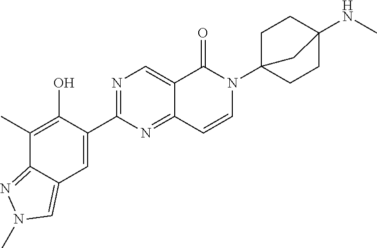 |
| 1480 | 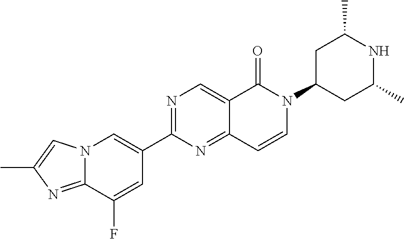 |
| 1481 | 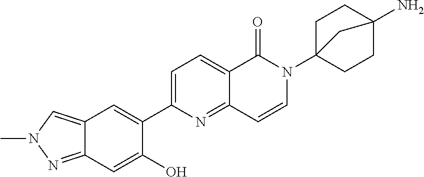 [SMILES: Cn1cc2cc(-c3ccc4c(=O)n(C56CCC(N)(CC5)C6)ccc4n3)c(O)cc2n1] |
| 1482 | 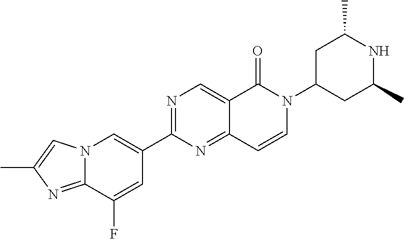 |
| 1483 | 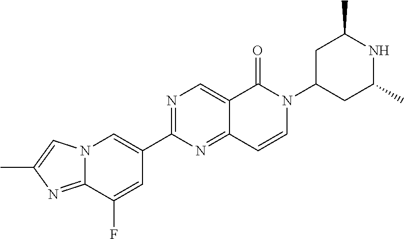 |
| 1484 | 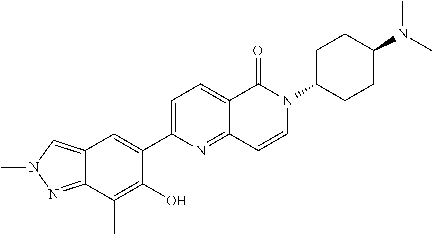 |
| 1485 | 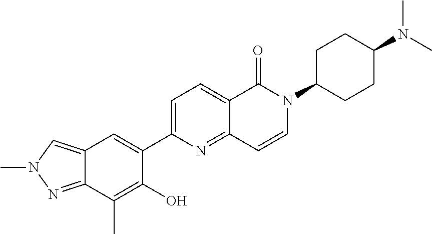 |
| 1486 | 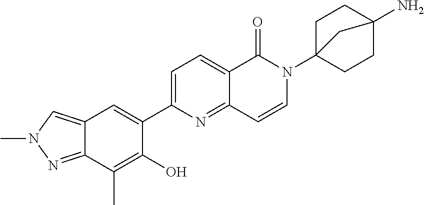 [SMILES: Cc1c(O)c(-c2ccc3c(=O)n(C45CCC(N)(CC4)C5)ccc3n2)cc2cn(C)nc12] |
| 1487 | 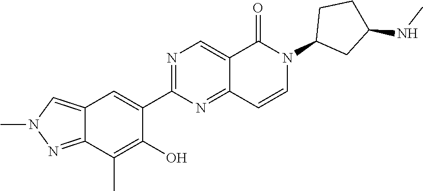 |
| 1488 | 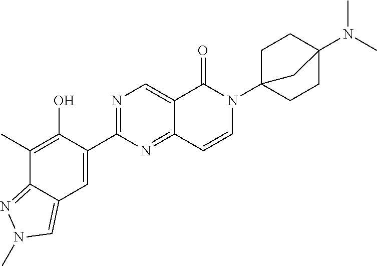 |
| 1489 | 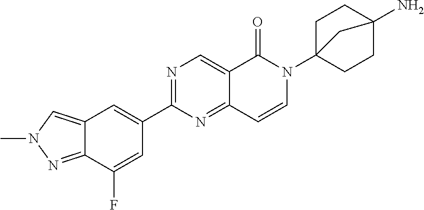 [SMILES: Cn1cc2cc(-c3ncc4c(=O)n(C56CCC(N)(CC5)C6)ccc4n3)cc(F)c2n1] |
| 1490 | 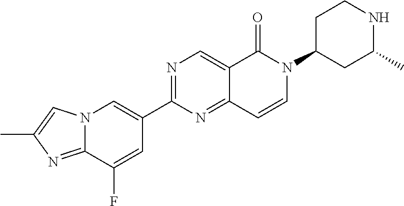 |
| 1491 | 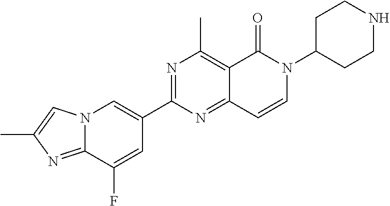 [SMILES: Cc1cn2cc(-c3nc(C)c4c(=O)n(C5CCNCC5)ccc4n3)cc(F)c2n1] |
| 1492 | 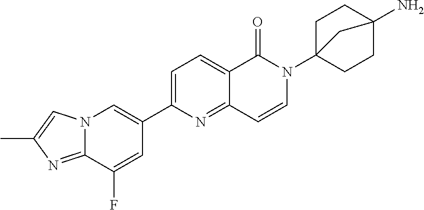 [SMILES: Cc1cn2cc(-c3ccc4c(=O)n(C56CCC(N)(CC5)C6)ccc4n3)cc(F)c2n1] |
| 1493 | 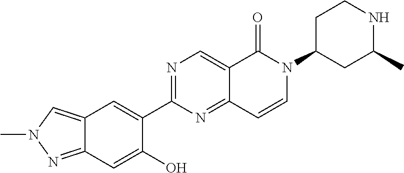 |
| 1494 | 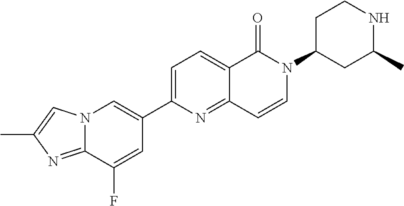 |
| 1495 | 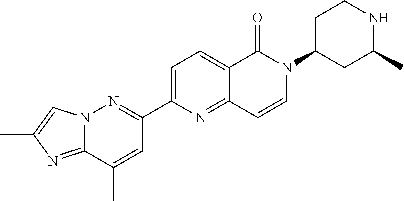 |
| 1496 | 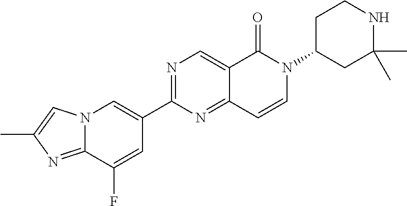 |
| 1497 | 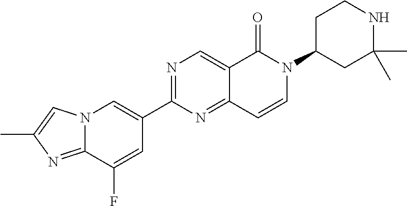 |
| 1498 | 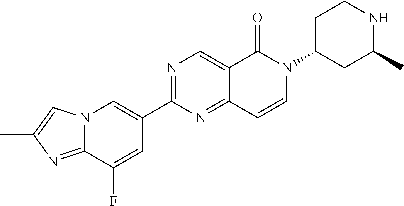 |
| 1499 | 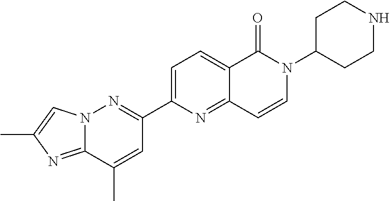 [SMILES: Cc1cn2nc(-c3ccc4c(=O)n(C5CCNCC5)ccc4n3)cc(C)c2n1] |
| 1500 | 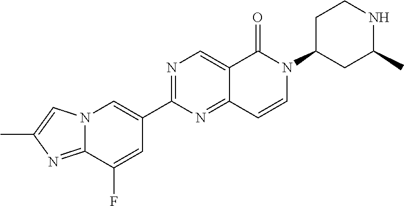 |
| 1501 | 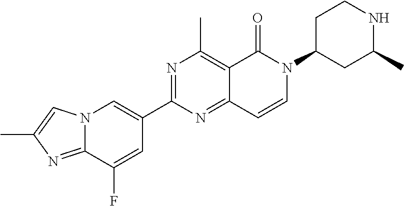 |
| 1502 | 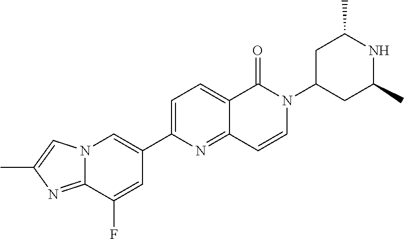 |
| 1503 | 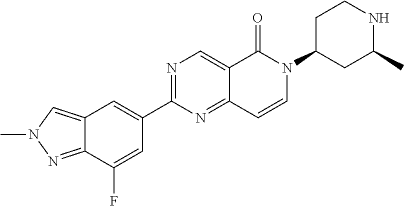 |
| 1504 | 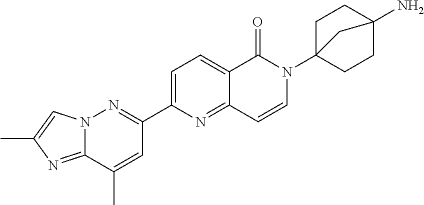 [SMILES: Cc1cn2nc(-c3ccc4c(=O)n(C56CCC(N)(CC5)C6)ccc4n3)cc(C)c2n1] |
| 1505 | 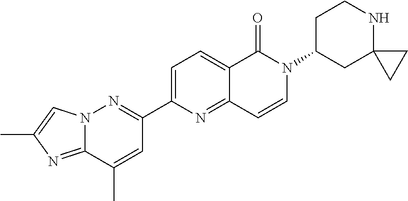 [SMILES: Cc1cn2nc(-c3ccc4c(=O)n([C@@H]5CCNC6(CC6)C5)ccc4n3)cc(C)c2n1] |
| 1506 | 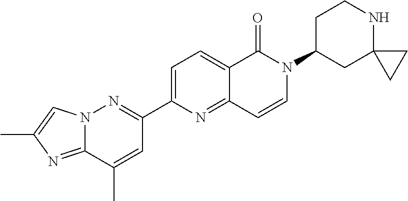 [SMILES: Cc1cn2nc(-c3ccc4c(=O)n([C@H]5CCNC6(CC6)C5)ccc4n3)cc(C)c2n1] |
| 1507 | 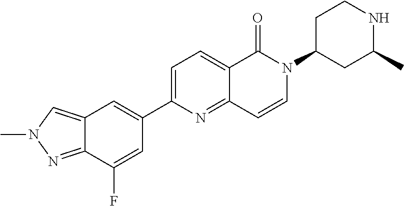 |
| 1508 | 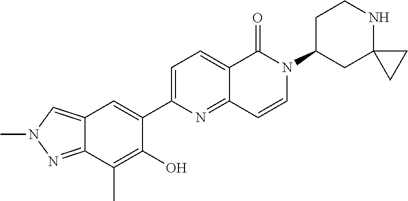 [SMILES: Cc1c(O)c(-c2ccc3c(=O)n([C@H]4CCNC5(CC5)C4)ccc3n2)cc2cn(C)nc12] |
| 1509 | 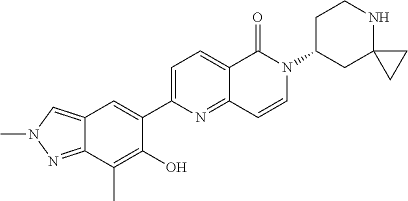 [SMILES: Cc1c(O)c(-c2ccc3c(=O)n([C@@H]4CCNC5(CC5)C4)ccc3n2)cc2cn(C)nc12] |
| 1510 | 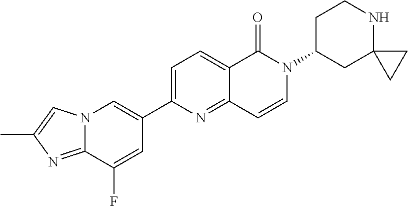 [SMILES: Cc1cn2cc(-c3ccc4c(=O)n([C@@H]5CCNC6(CC6)C5)ccc4n3)cc(F)c2n1] |
| 1511 | 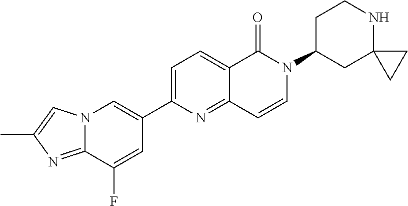 [SMILES: Cc1cn2cc(-c3ccc4c(=O)n([C@H]5CCNC6(CC6)C5)ccc4n3)cc(F)c2n1] |
| 1512 | 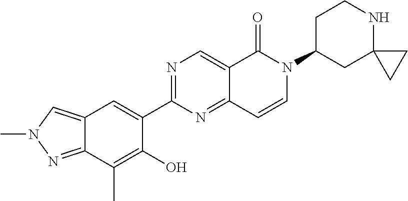 [SMILES: Cc1c(O)c(-c2ncc3c(=O)n([C@H]4CCNC5(CC5)C4)ccc3n2)cc2cn(C)nc12] |
| 1513 | 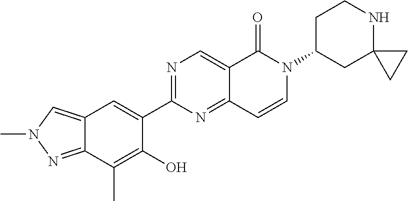 [SMILES: Cc1c(O)c(-c2ncc3c(=O)n([C@@H]4CCNC5(CC5)C4)ccc3n2)cc2cn(C)nc12] |
| 1514 | 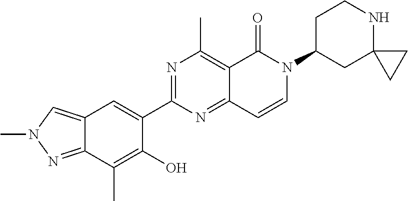 [SMILES: Cc1c(O)c(-c2nc(C)c3c(=O)n([C@H]4CCNC5(CC5)C4)ccc3n2)cc2cn(C)nc12] |
| 1515 | 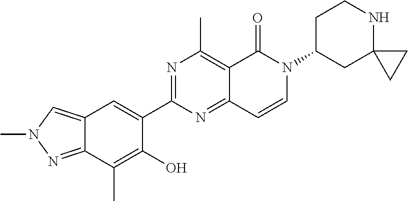 [SMILES: Cc1c(O)c(-c2nc(C)c3c(=O)n([C@@H]4CCNC5(CC5)C4)ccc3n2)cc2cn(C)nc12] |
| 1516 | 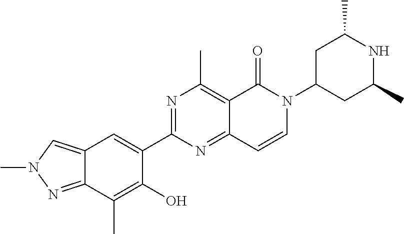 |
| 1517 | 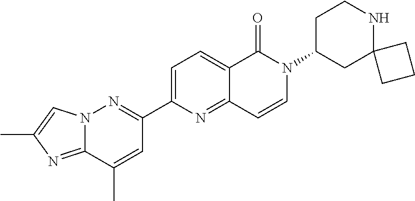 [SMILES: Cc1cn2nc(-c3ccc4c(=O)n([C@@H]5CCNC6(CCC6)C5)ccc4n3)cc(C)c2n1] |
| 1518 | 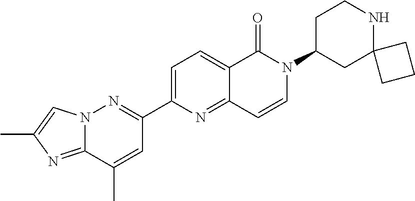 [SMILES: Cc1cn2nc(-c3ccc4c(=O)n([C@H]5CCNC6(CCC6)C5)ccc4n3)cc(C)c2n1] |
| 1519 | 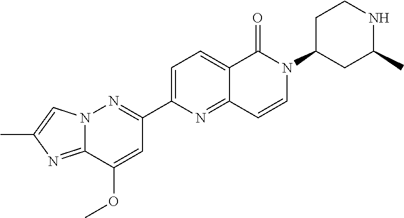 |
| 1520 | 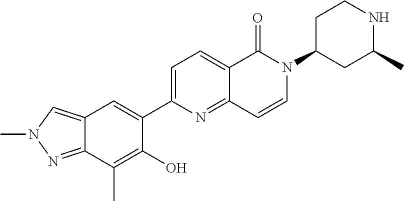 |
| 1521 | 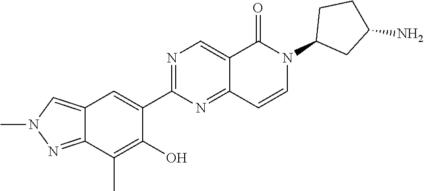 [SMILES: Cc1c(O)c(-c2ncc3c(=O)n([C@H]4CC[C@H](N)C4)ccc3n2)cc2cn(C)nc12] |
| 1522 | 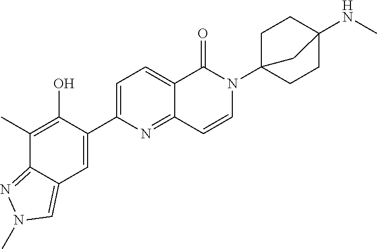 |
| 1523 | 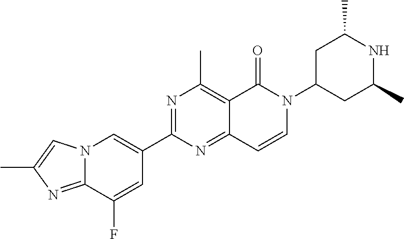 |
| 1524 | 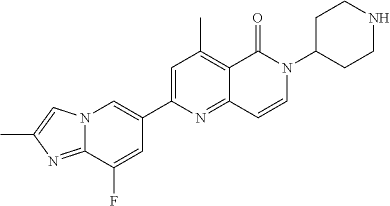 [SMILES: Cc1cn2cc(-c3cc(C)c4c(=O)n(C5CCNCC5)ccc4n3)cc(F)c2n1] |
| 1525 | 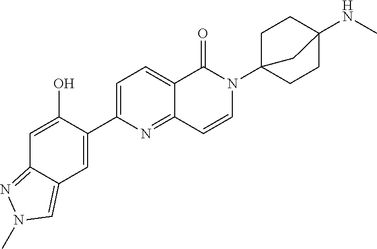 |
| 1526 | 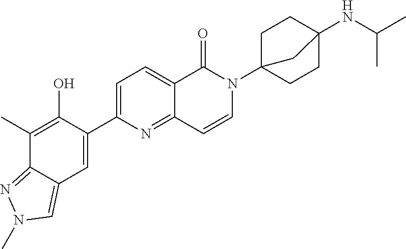 [SMILES: Cc1c(O)c(-c2ccc3c(=O)n(C45CCC(NC(C)C)(CC4)C5)ccc3n2)cc2cn(C)nc12] |

In some embodiments, the compound of Formula (V) is a compound of Formula (V-a):

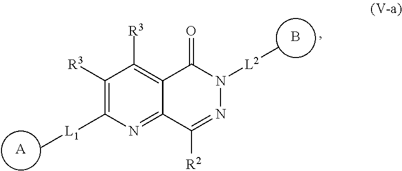

or a pharmaceutically acceptable salt, solvate, hydrate, tautomer, or stereoisomer thereof, wherein A, B, L¹, L², R², R³, and subvariables thereof are as defined herein.

In some embodiments, the compound of Formula (V) is a compound of Formula (V-b):

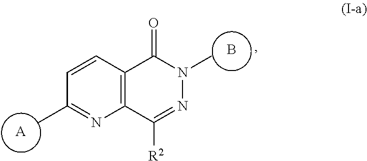

or a pharmaceutically acceptable salt, solvate, hydrate, tautomer, or stereoisomer thereof, wherein A, B, R², and subvariables thereof are as defined herein.

In some embodiments, the compound of Formula (V) is a compound of Formula (V-c):

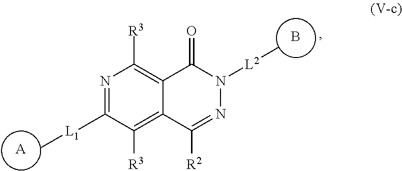

or a pharmaceutically acceptable salt, solvate, hydrate, tautomer, or stereoisomer thereof, wherein A, B, L¹, L², R², R³, and subvariables thereof are as defined herein.

In some embodiments, the compound of Formula (V) is a compound of Formula (V-d):

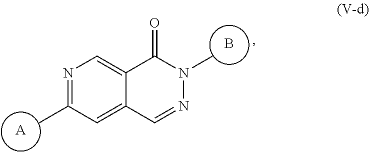

or a pharmaceutically acceptable salt, solvate, hydrate, tautomer, or stereoisomer thereof, wherein A, B, and subvariables thereof are as defined herein.

In some embodiments, the compound of Formula (V) is a compound of Formula (V-e):

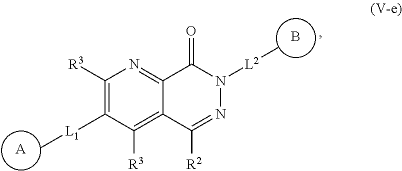

or a pharmaceutically acceptable salt, solvate, hydrate, tautomer, or stereoisomer thereof, wherein A, B, L¹, L², R², R³, and subvariables thereof are as defined herein.

In some embodiments, the compound of Formula (V) is a compound of Formula (V-f):

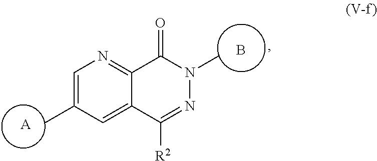

or a pharmaceutically acceptable salt, solvate, hydrate, tautomer, or stereoisomer thereof, wherein A, B, R², and subvariables thereof are as defined herein.

In some embodiments, the compound of Formula (V) is a compound provided in Table 5.

TABLE 5
Exemplary Compounds of Formula (V)

| Cmpd | |
|---|---|
| No. | Structure |
| 1247 | 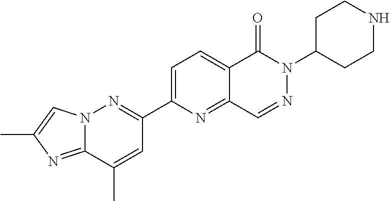 [SMILES: Cc1cn2nc(-c3ccc4c(=O)n(C5CCNCC5)ncc4n3)cc(C)c2n1] |
| 1250 | 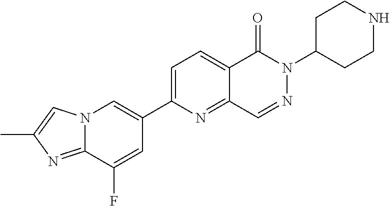 [SMILES: Cc1cn2cc(-c3ccc4c(=O)n(C5CCNCC5)ncc4n3)cc(F)c2n1] |
| 1251 | 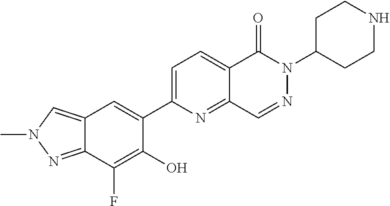 [SMILES: Cn1cc2cc(-c3ccc4c(=O)n(C5CCNCC5)ncc4n3)c(O)c(F)c2n1] |
| 1252 | 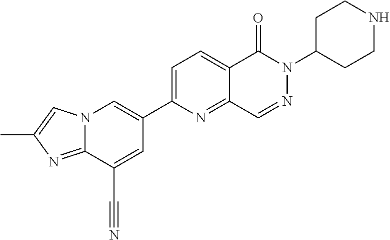 [SMILES: Cc1cn2cc(-c3ccc4c(=O)n(C5CCNCC5)ncc4n3)cc(C#N)c2n1] |
| 1253 | 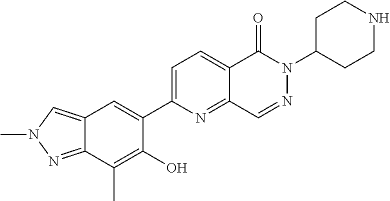 [SMILES: Cc1c(O)c(-c2ccc3c(=O)n(C4CCNCC4)ncc3n2)cc2cn(C)nc12] |
| 1254 | 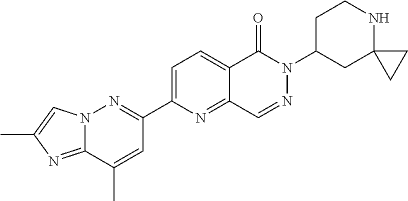 [SMILES: Cc1cn2nc(-c3ccc4c(=O)n(C5CCNC6(CC6)C5)ncc4n3)cc(C)c2n1] |
| 1255 | 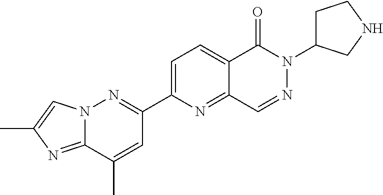 [SMILES: Cc1cn2nc(-c3ccc4c(=O)n(C5CCNC5)ncc4n3)cc(C)c2n1] |
| 1256 | 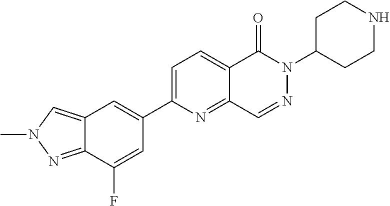 [SMILES: Cn1cc2cc(-c3ccc4c(=O)n(C5CCNCC5)ncc4n3)cc(F)c2n1] |
| 1257 | 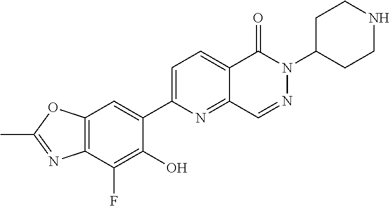 [SMILES: Cc1nc2c(F)c(O)c(-c3ccc4c(=O)n(C5CCNCC5)ncc4n3)cc2o1] |
| 1258 | 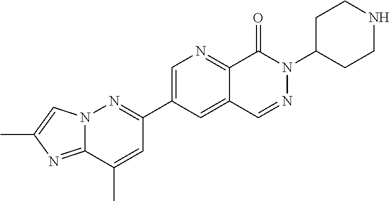 [SMILES: Cc1cn2nc(-c3cnc4c(=O)n(C5CCNCC5)ncc4c3)cc(C)c2n1] |
| 1259 | 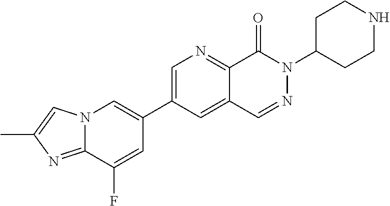 [SMILES: Cc1cn2cc(-c3cnc4c(=O)n(C5CCNCC5)ncc4c3)cc(F)c2n1] |
| 1260 | 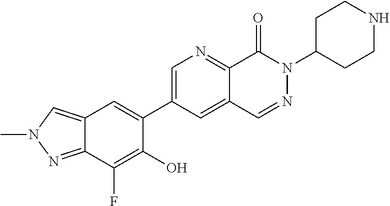 [SMILES: Cn1cc2cc(-c3cnc4c(=O)n(C5CCNCC5)ncc4c3)c(O)c(F)c2n1] |
| 1261 | 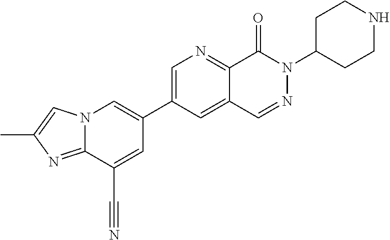 [SMILES: Cc1cn2cc(-c3cnc4c(=O)n(C5CCNCC5)ncc4c3)cc(C#N)c2n1] |
| 1262 | 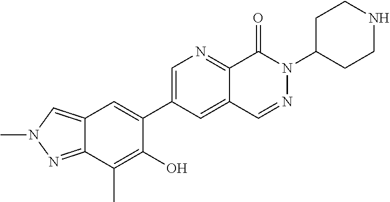 [SMILES: Cc1c(O)c(-c2cnc3c(=O)n(C4CCNCC4)ncc3c2)cc2cn(C)nc12] |
| 1263 | 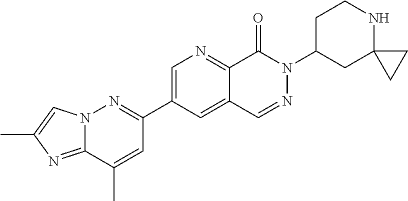 [SMILES: Cc1cn2nc(-c3cnc4c(=O)n(C5CCNC6(CC6)C5)ncc4c3)cc(C)c2n1] |
| 1264 | 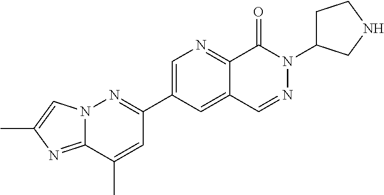 [SMILES: Cc1cn2nc(-c3cnc4c(=O)n(C5CCNC5)ncc4c3)cc(C)c2n1] |
| 1265 | 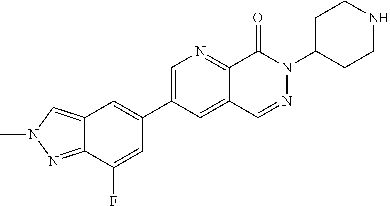 [SMILES: Cn1cc2cc(-c3cnc4c(=O)n(C5CCNCC5)ncc4c3)cc(F)c2n1] |
| 1266 | 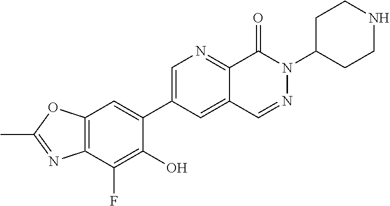 [SMILES: Cc1nc2c(F)c(O)c(-c3cnc4c(=O)n(C5CCNCC5)ncc4c3)cc2o1] |
| 1267 | 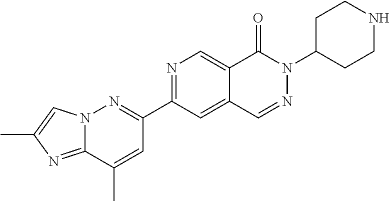 [SMILES: Cc1cn2nc(-c3cc4cnn(C5CCNCC5)c(=O)c4cn3)cc(C)c2n1] |
| 1268 | 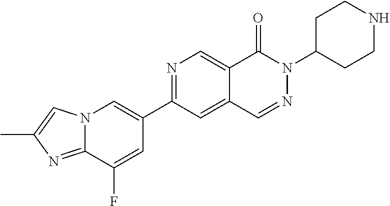 [SMILES: Cc1cn2cc(-c3cc4cnn(C5CCNCC5)c(=O)c4cn3)cc(F)c2n1] |
| 1269 | 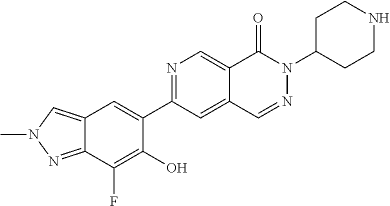 [SMILES: Cn1cc2cc(-c3cc4cnn(C5CCNCC5)c(=O)c4cn3)c(O)c(F)c2n1] |
| 1270 | 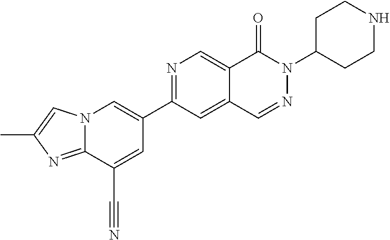 [SMILES: Cc1cn2cc(-c3cc4cnn(C5CCNCC5)c(=O)c4cn3)cc(C#N)c2n1] |
| 1271 | 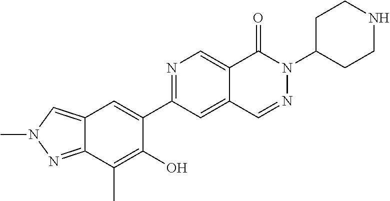 [SMILES: Cc1c(O)c(-c2cc3cnn(C4CCNCC4)c(=O)c3cn2)cc2cn(C)nc12] |
| 1272 | 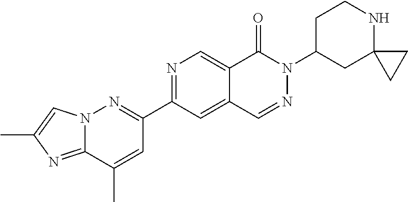 [SMILES: Cc1cn2nc(-c3cc4cnn(C5CCNC6(CC6)C5)c(=O)c4cn3)cc(C)c2n1] |
| 1273 | 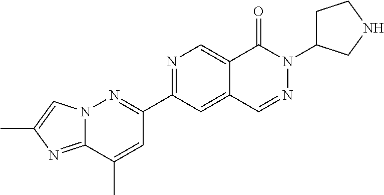 [SMILES: Cc1cn2nc(-c3cc4cnn(C5CCNC5)c(=O)c4cn3)cc(C)c2n1] |
| 1274 | 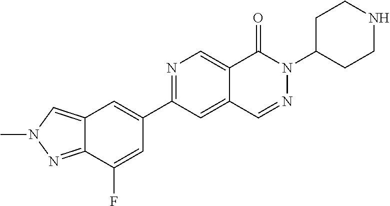 [SMILES: Cn1cc2cc(-c3cc4cnn(C5CCNCC5)c(=O)c4cn3)cc(F)c2n1] |
| 1275 | 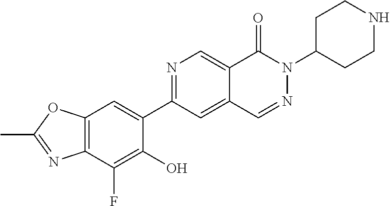 [SMILES: Cc1nc2c(F)c(O)c(-c3cc4cnn(C5CCNCC5)c(=O)c4cn3)cc2o1] |
| 1298 | 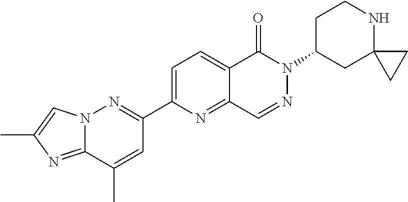 [SMILES: Cc1cn2nc(-c3ccc4c(=O)n([C@@H]5CCNC6(CC6)C5)ncc4n3)cc(C)c2n1] |
| 1306 | 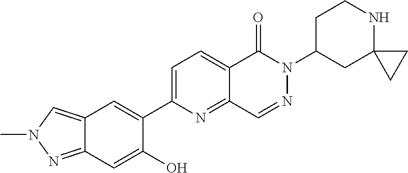 [SMILES: Cn1cc2cc(-c3ccc4c(=O)n(C5CCNC6(CC6)C5)ncc4n3)c(O)cc2n1] |
| 1307 | 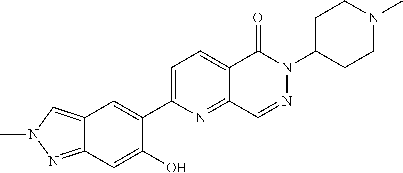 |
| 1308 | 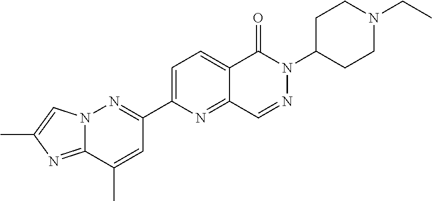 [SMILES: CCN1CCC(n2ncc3nc(-c4cc(C)c5nc(C)cn5n4)ccc3c2=O)CC1] |
| 1309 | 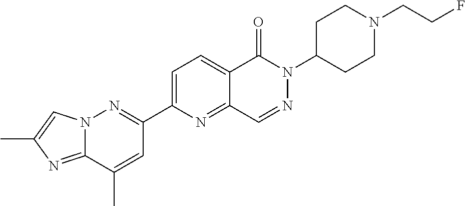 [SMILES: Cc1cn2nc(-c3ccc4c(=O)n(C5CCN(CCF)CC5)ncc4n3)cc(C)c2n1] |
| 1310 | 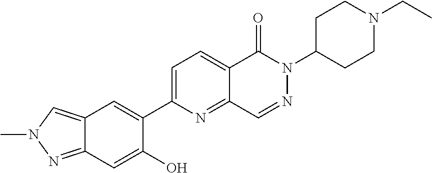 [SMILES: CCN1CCC(n2ncc3nc(-c4cc5cn(C)nc5cc4O)ccc3c2=O)CC1] |
| 1311 | 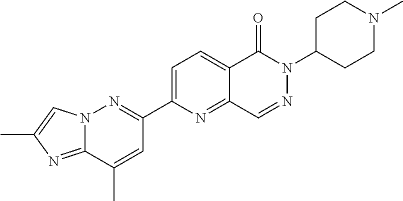 |
| 1312 | 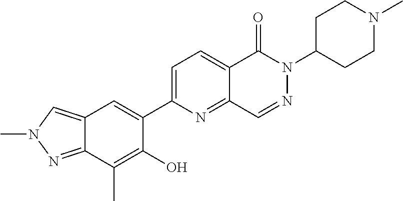 |
| 1313 | 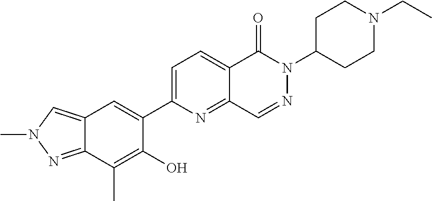 [SMILES: CCN1CCC(n2ncc3nc(-c4cc5cn(C)nc5c(C)c4O)ccc3c2=O)CC1] |
| 1314 | 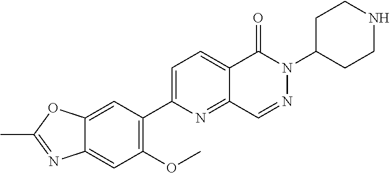 [SMILES: COc1cc2nc(C)oc2cc1-c1ccc2c(=O)n(C3CCNCC3)ncc2n1] |
| 1315 | 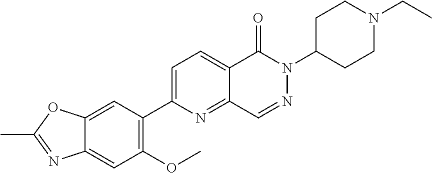 [SMILES: CCN1CCC(n2ncc3nc(-c4cc5oc(C)nc5cc4OC)ccc3c2=O)CC1] |
| 1316 | 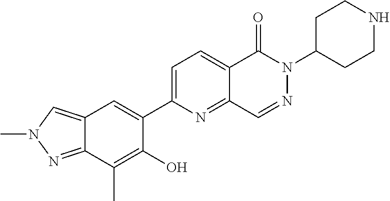 [SMILES: Cc1c(O)c(-c2ccc3c(=O)n(C4CCNCC4)ncc3n2)cc2cn(C)nc12] |
| 1317 | 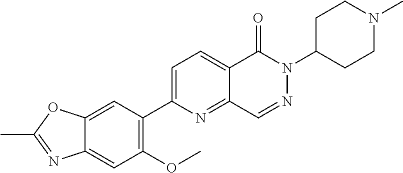 |
| 1318 | 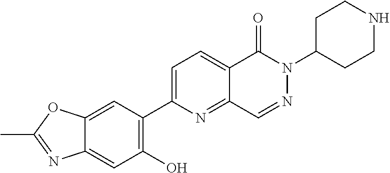 [SMILES: Cc1nc2cc(O)c(-c3ccc4c(=O)n(C5CCNCC5)ncc4n3)cc2o1] |
| 1319 | 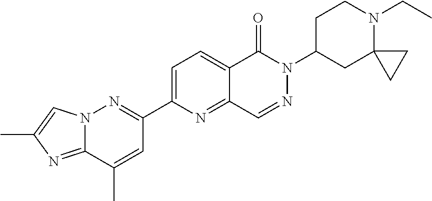 [SMILES: CCN1CCC(n2ncc3nc(-c4cc(C)c5nc(C)cn5n4)ccc3c2=O)CC12CC2] |
| 1411 | 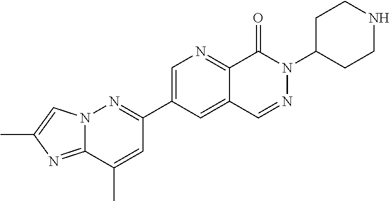 [SMILES: Cc1cn2nc(-c3cnc4c(=O)n(C5CCNCC5)ncc4c3)cc(C)c2n1] |
| 1412 | 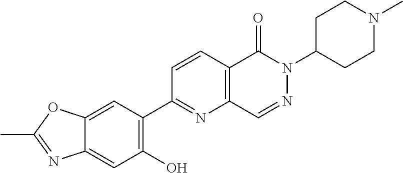 |
| 1413 | 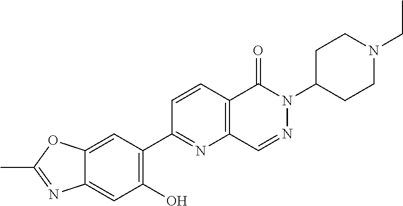 [SMILES: CCN1CCC(n2ncc3nc(-c4cc5oc(C)nc5cc4O)ccc3c2=O)CC1] |
| 1414 | 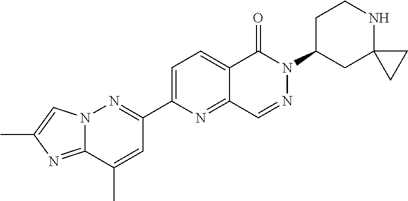 [SMILES: Cc1cn2nc(-c3ccc4c(=O)n([C@H]5CCNC6(CC6)C5)ncc4n3)cc(C)c2n1] |
| 1415 | 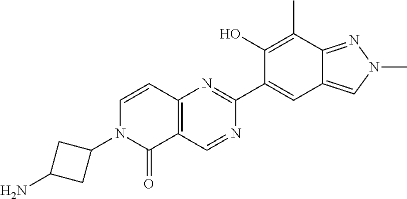 [SMILES: Cc1c(O)c(-c2ncc3c(=O)n(C4CC(N)C4)ccc3n2)cc2cn(C)nc12] |
| 1416 | 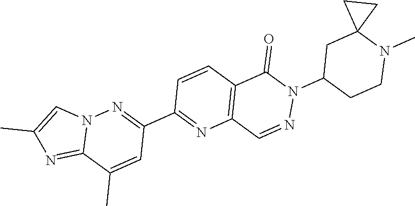 |
| 1417 | 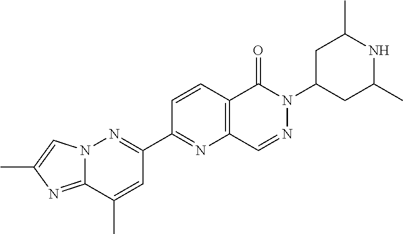 |
| 1418 | 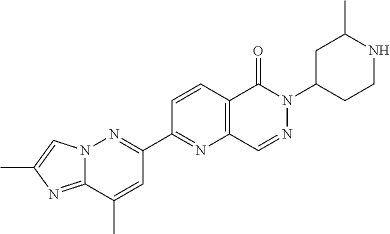 [SMILES: Cc1cn2nc(-c3ccc4c(=O)n(C5CCNC(C)C5)ncc4n3)cc(C)c2n1] |
| 1419 | 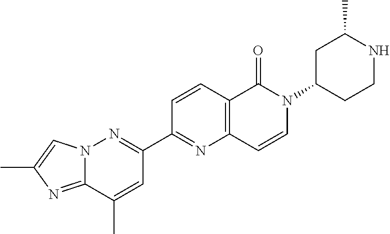 [SMILES: Cc1cn2nc(-c3ccc4c(=O)n([C@H]5CCN[C@@H](C)C5)ccc4n3)cc(C)c2n1] |
| 1420 | 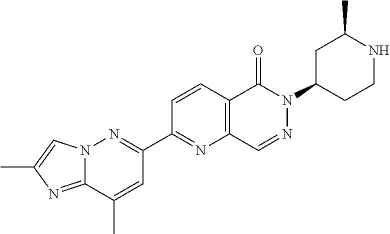 [SMILES: Cc1cn2nc(-c3ccc4c(=O)n([C@@H]5CCN[C@H](C)C5)ncc4n3)cc(C)c2n1] |
| 1421 | 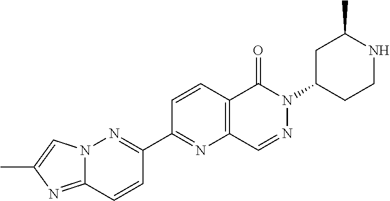 [SMILES: Cc1cn2nc(-c3ccc4c(=O)n([C@H]5CCN[C@H](C)C5)ncc4n3)ccc2n1] |
| 1422 | 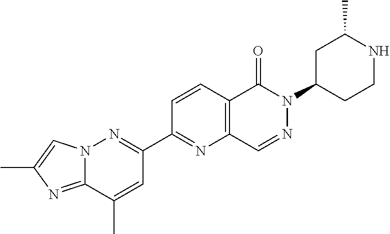 [SMILES: Cc1cn2nc(-c3ccc4c(=O)n([C@@H]5CCN[C@@H](C)C5)ncc4n3)cc(C)c2n1] |
| 1423 | 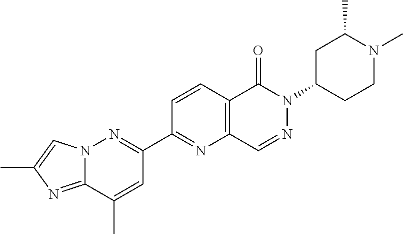 |
| 1424 | 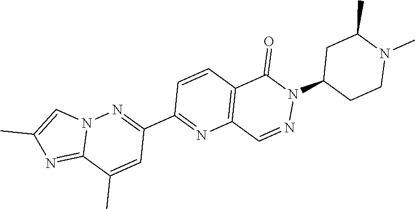 |
| 1425 | 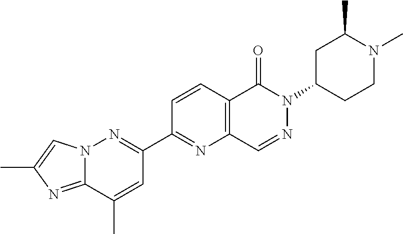 |
| 1426 | 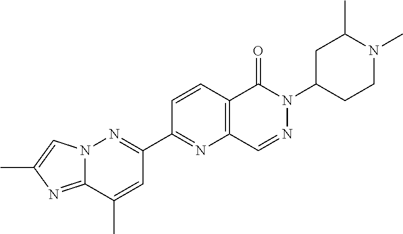 |
| 1427 | 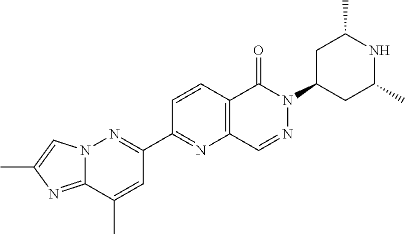 |
| 1527 | 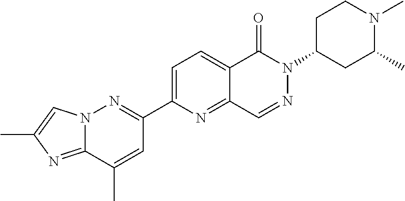 |
| 1528 | 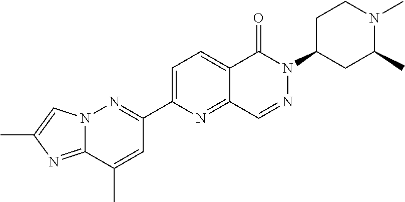 |
| 1529 | 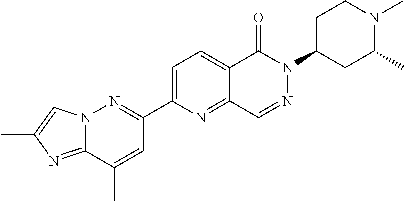 |
| 1530 | 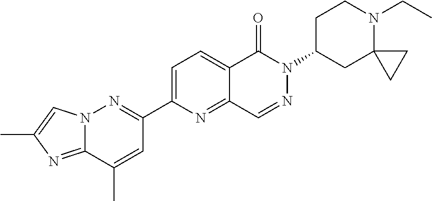 [SMILES: CCN1CC[C@@H](n2ncc3nc(-c4cc(C)c5nc(C)cn5n4)ccc3c2=O)CC12CC2] |
| 1531 | 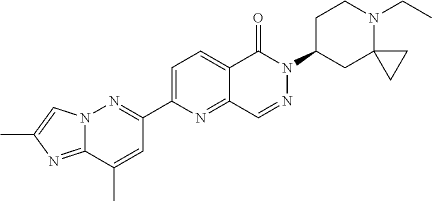 [SMILES: CCN1CC[C@H](n2ncc3nc(-c4cc(C)c5nc(C)cn5n4)ccc3c2=O)CC12CC2] |
| 1532 | 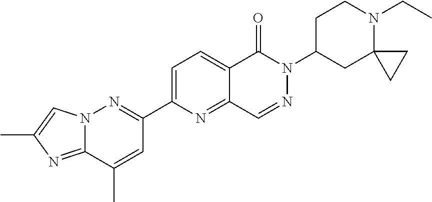 [SMILES: CCN1CCC(n2ncc3nc(-c4cc(C)c5nc(C)cn5n4)ccc3c2=O)CC12CC2] |
| 1533 | 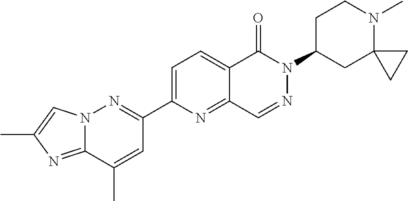 [SMILES: Cc1cn2nc(-c3ccc4c(=O)n([C@H]5CCN(C)C6(CC6)C5)ncc4n3)cc(C)c2n1] |
| 1534 | 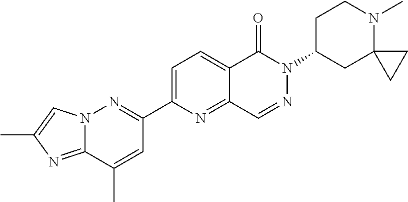 [SMILES: Cc1cn2nc(-c3ccc4c(=O)n([C@@H]5CCN(C)C6(CC6)C5)ncc4n3)cc(C)c2n1] |
| 1535 | 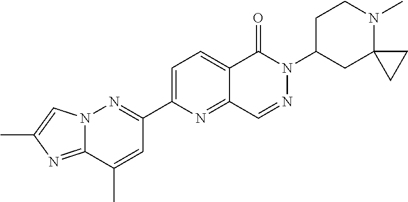 [SMILES: Cc1cn2nc(-c3ccc4c(=O)n(C5CCN(C)C6(CC6)C5)ncc4n3)cc(C)c2n1] |
| 1536 | 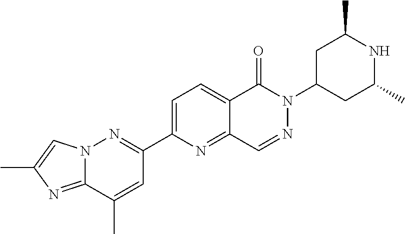 |
| 1537 | 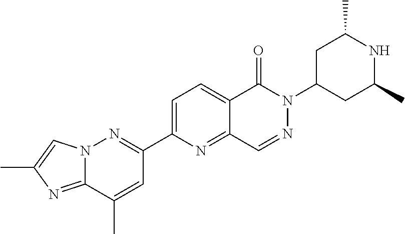 |
| 1538 | 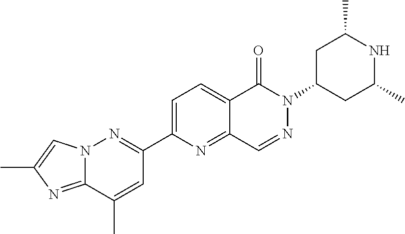 |
| 1539 | 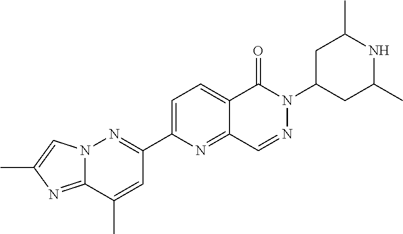 |
| 1540 | 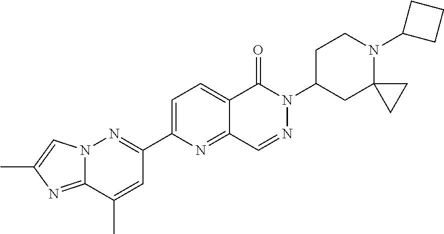 [SMILES: Cc1cn2nc(-c3ccc4c(=O)n(C5CCN(C6CCC6)C6(CC6)C5)ncc4n3)cc(C)c2n1] |
| 1541 | 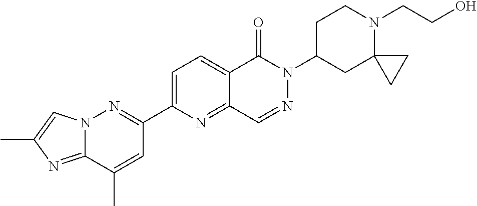 [SMILES: Cc1cn2nc(-c3ccc4c(=O)n(C5CCN(CCO)C6(CC6)C5)ncc4n3)cc(C)c2n1] |
| 1542 | 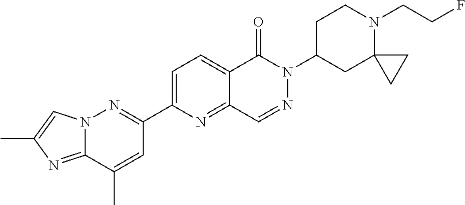 [SMILES: Cc1cn2nc(-c3ccc4c(=O)n(C5CCN(CCF)C6(CC6)C5)ncc4n3)cc(C)c2n1] |
| 1543 | 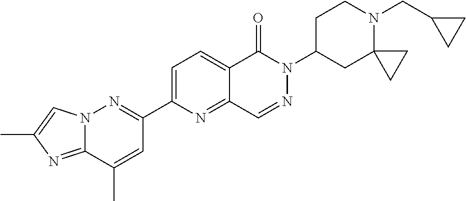 [SMILES: Cc1cn2nc(-c3ccc4c(=O)n(C5CCN(CC6CC6)C6(CC6)C5)ncc4n3)cc(C)c2n1] |
| 1544 | 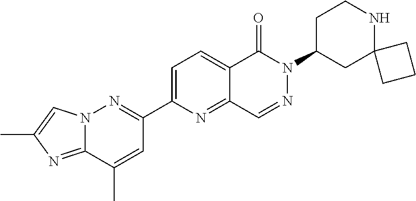 [SMILES: Cc1cn2nc(-c3ccc4c(=O)n([C@H]5CCNC6(CCC6)C5)ncc4n3)cc(C)c2n1] |
| 1545 | 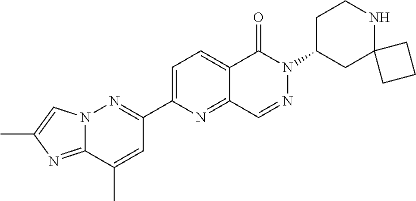 [SMILES: Cc1cn2nc(-c3ccc4c(=O)n([C@@H]5CCNC6(CCC6)C5)ncc4n3)cc(C)c2n1] |
| 1546 | 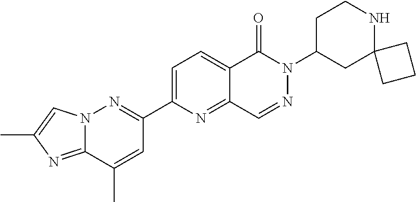 [SMILES: Cc1cn2nc(-c3ccc4c(=O)n(C5CCNC6(CCC6)C5)ncc4n3)cc(C)c2n1] |
| 1547 | 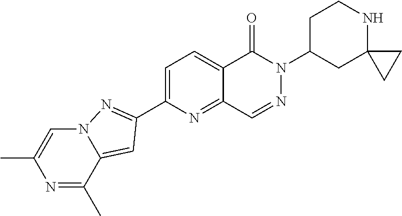 [SMILES: Cc1cn2nc(-c3ccc4c(=O)n(C5CCNC6(CC6)C5)ncc4n3)cc2c(C)n1] |
| 1548 | 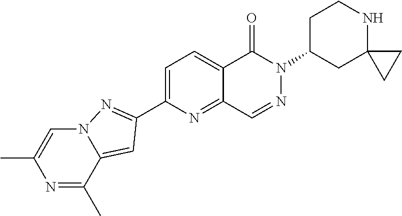 [SMILES: Cc1cn2nc(-c3ccc4c(=O)n([C@@H]5CCNC6(CC6)C5)ncc4n3)cc2c(C)n1] |
| 1549 | 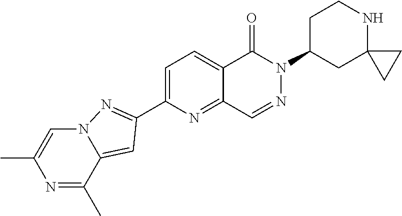 [SMILES: Cc1cn2nc(-c3ccc4c(=O)n([C@H]5CCNC6(CC6)C5)ncc4n3)cc2c(C)n1] |
| 1550 | 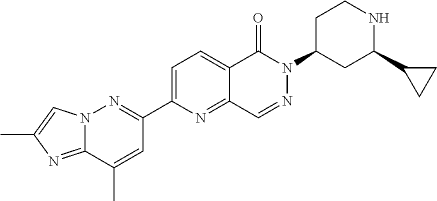 [SMILES: Cc1cn2nc(-c3ccc4c(=O)n([C@H]5CCN[C@@H](C6CC6)C5)ncc4n3)cc(C)c2n1] |
| 1551 | 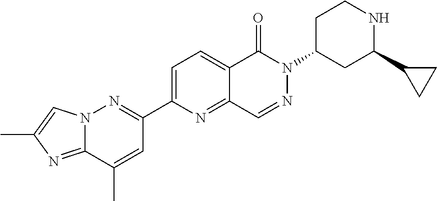 [SMILES: Cc1cn2nc(-c3ccc4c(=O)n([C@@H]5CCN[C@@H](C6CC6)C5)ncc4n3)cc(C)c2n1] |
| 1552 | 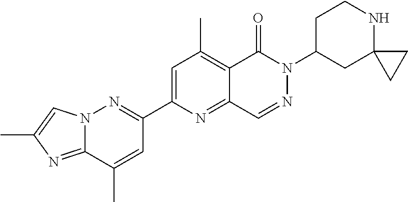 [SMILES: Cc1cn2nc(-c3cc(C)c4c(=O)n(C5CCNC6(CC6)C5)ncc4n3)cc(C)c2n1] |
| 1553 | 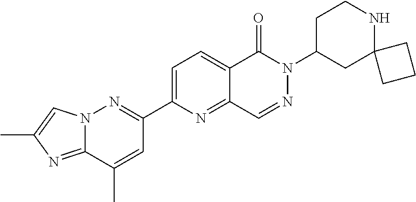 [SMILES: Cc1cn2nc(-c3ccc4c(=O)n(C5CCNC6(CCC6)C5)ncc4n3)cc(C)c2n1] |

In some embodiments, the compound of Formula (VI) is a compound of Formula (VI-a):

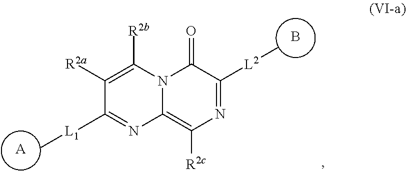

or a pharmaceutically acceptable salt, solvate, hydrate, tautomer, or stereoisomer thereof, wherein A, B, L¹, L², R^{2a}, R^{2b}, R^{2c}, and subvariables thereof are as defined herein.

In some embodiments, the compound of Formula (VI) is a compound of Formula (VI-b):

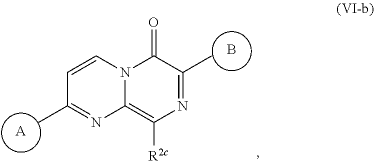

or a pharmaceutically acceptable salt, solvate, hydrate, tautomer, or stereoisomer thereof, wherein A, B, R^{2c}, and subvariables thereof are as defined herein.

In some embodiments, the compound of Formula (VI) is a compound of Formula (VI-c):

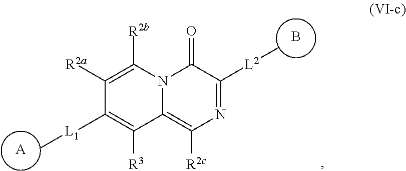

or a pharmaceutically acceptable salt, solvate, hydrate, tautomer, or stereoisomer thereof, wherein A, B, L¹, L², R^{2a}, R^{2b}, R^{2c}, R³, and subvariables thereof are as defined herein.

In some embodiments, the compound of Formula (VI) is a compound of Formula (VI-d):

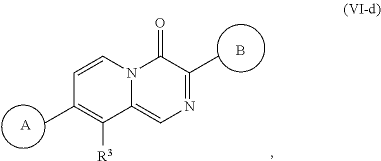

or a pharmaceutically acceptable salt, solvate, hydrate, tautomer, or stereoisomer thereof, wherein A, B, R³, and subvariables thereof are as defined herein.

In some embodiments, the compound of Formula (VI) is a compound of Formula (VI-e):

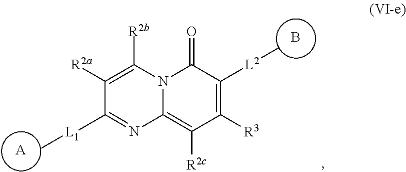

or a pharmaceutically acceptable salt, solvate, hydrate, tautomer, or stereoisomer thereof, wherein A, B, L¹, L², R^{2a}, R^{2b}, R^{2c}, R³, and subvariables thereof are as defined herein.

In some embodiments, the compound of Formula (VI) is a compound of Formula (VI-f):

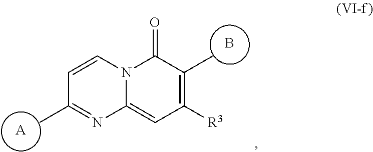

or a pharmaceutically acceptable salt, solvate, hydrate, tautomer, or stereoisomer thereof, wherein A, B, R³, and subvariables thereof are as defined herein.

In some embodiments, the compound of Formula (VI) is a compound of Formula (VI-g):

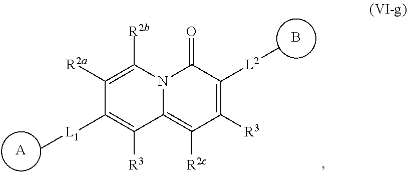

or a pharmaceutically acceptable salt, solvate, hydrate, tautomer, or stereoisomer thereof, wherein A, B, L¹, L², R^{2a}, R^{2b}, R^{2c}, R³, and subvariables thereof are as defined herein.

In some embodiments, the compound of Formula (VI) is a compound of Formula (VI-h):

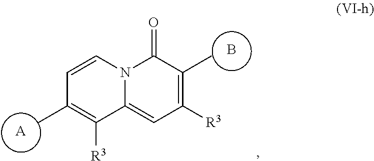

or a pharmaceutically acceptable salt, solvate, hydrate, tautomer, or stereoisomer thereof, wherein A, B, R³, and subvariables thereof are as defined herein.

In some embodiments, the compound of Formula (VI) is a compound provided in Table 6.

TABLE 6
Exemplary Compounds of Formula (VI)

| Cmpd | |
|---|---|
| No. | Structure |
| 1112 | 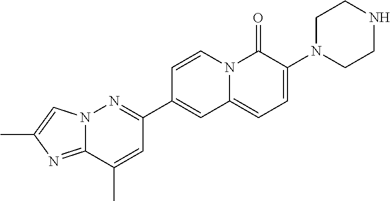 [SMILES: Cc1cn2nc(-c3ccn4c(=O)c(N5CCNCC5)ccc4c3)cc(C)c2n1] |
| 1153 | 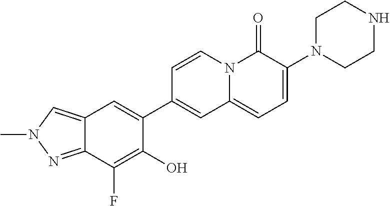 [SMILES: Cn1cc2cc(-c3ccn4c(=O)c(N5CCNCC5)ccc4c3)c(O)c(F)c2n1] |
| 1154 | 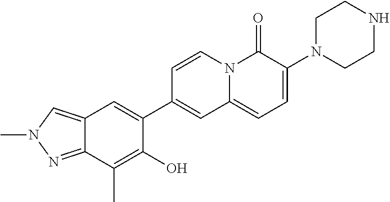 [SMILES: Cc1c(O)c(-c2ccn3c(=O)c(N4CCNCC4)ccc3c2)cc2cn(C)nc12] |
| 1155 | 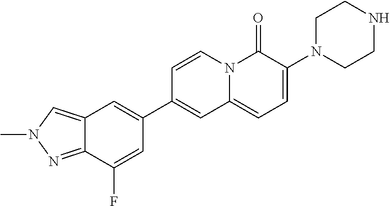 [SMILES: Cn1cc2cc(-c3ccn4c(=O)c(N5CCNCC5)ccc4c3)cc(F)c2n1] |
| 1156 | 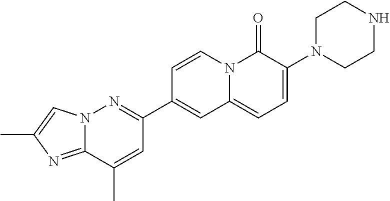 [SMILES: Cc1cn2nc(-c3ccn4c(=O)c(N5CCNCC5)ccc4c3)cc(C)c2n1] |
| 1157 | 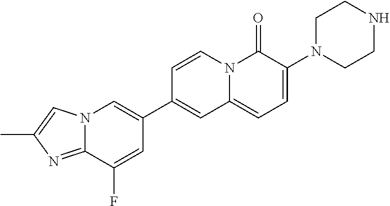 [SMILES: Cc1cn2cc(-c3ccn4c(=O)c(N5CCNCC5)ccc4c3)cc(F)c2n1] |
| 1158 | 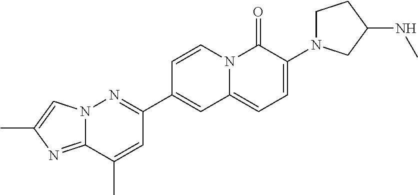 |
| 1159 | 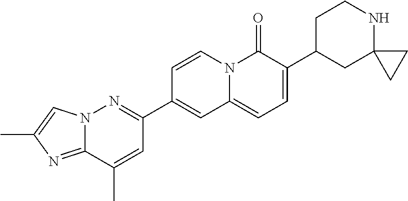 [SMILES: Cc1cn2nc(-c3ccn4c(=O)c(C5CCNC6(CC6)C5)ccc4c3)cc(C)c2n1] |
| 1164 | 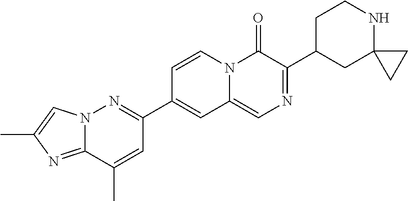 [SMILES: Cc1cn2nc(-c3ccn4c(=O)c(C5CCNC6(CC6)C5)ncc4c3)cc(C)c2n1] |
| 1171 | 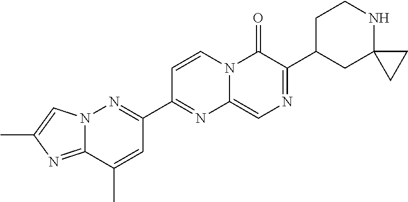 [SMILES: Cc1cn2nc(-c3ccn4c(=O)c(C5CCNC6(CC6)C5)ncc4n3)cc(C)c2n1] |
| 1172 | 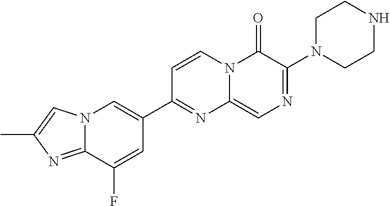 [SMILES: Cc1cn2cc(-c3ccn4c(=O)c(N5CCNCC5)ncc4n3)cc(F)c2n1] |
| 1173 | 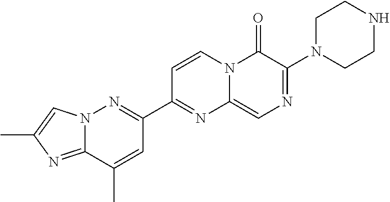 [SMILES: Cc1cn2nc(-c3ccn4c(=O)c(N5CCNCC5)ncc4n3)cc(C)c2n1] |
| 1174 | 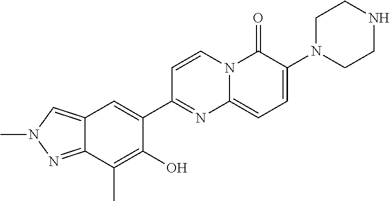 [SMILES: Cc1c(O)c(-c2ccn3c(=O)c(N4CCNCC4)ccc3n2)cc2cn(C)nc12] |
| 1175 | 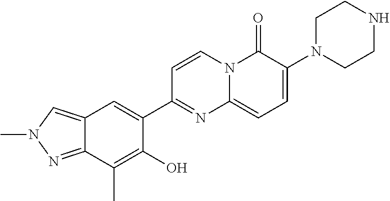 [SMILES: Cc1c(O)c(-c2ccn3c(=O)c(N4CCNCC4)ccc3n2)cc2cn(C)nc12] |
| 1176 | 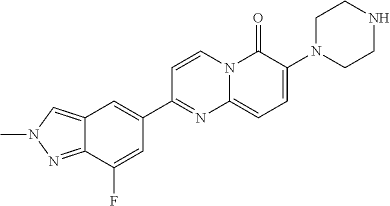 [SMILES: Cn1cc2cc(-c3ccn4c(=O)c(N5CCNCC5)ccc4n3)cc(F)c2n1] |
| 1177 | 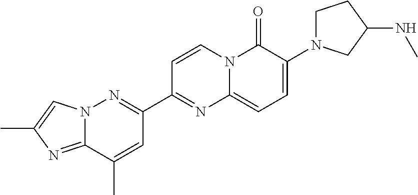 |
| 1178 | 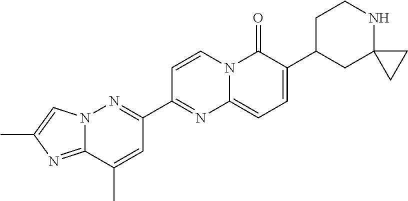 [SMILES: Cc1cn2nc(-c3ccn4c(=O)c(C5CCNC6(CC6)C5)ccc4n3)cc(C)c2n1] |
| 1179 | 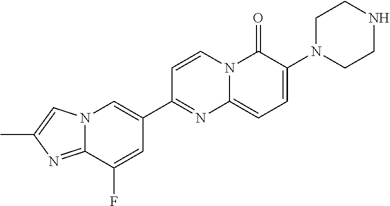 [SMILES: Cc1cn2cc(-c3ccn4c(=O)c(N5CCNCC5)ccc4n3)cc(F)c2n1] |
| 1180 | 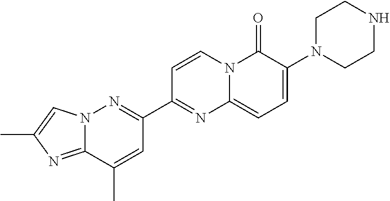 [SMILES: Cc1cn2nc(-c3ccn4c(=O)c(N5CCNCC5)ccc4n3)cc(C)c2n1] |
| 1181 | 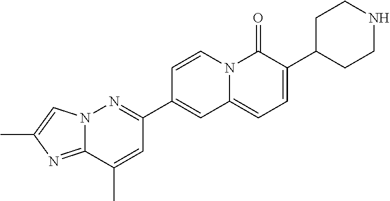 [SMILES: Cc1cn2nc(-c3ccn4c(=O)c(C5CCNCC5)ccc4c3)cc(C)c2n1] |
| 1182 | 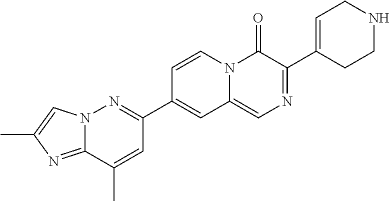 [SMILES: Cc1cn2nc(-c3ccn4c(=O)c(C5=CCNCC5)ncc4c3)cc(C)c2n1] |

In some embodiments, the compound of Formula (VII) is a compound provided in Table 7.

TABLE 7
Exemplary Compounds of Formula (VII)

| Cmpd | |
|---|---|
| No. | Structure |
| 1248 | 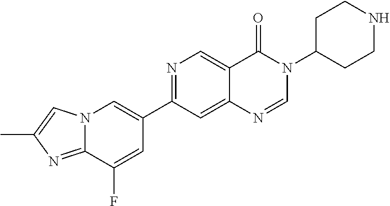 [SMILES: Cc1cn2cc(-c3cc4ncn(C5CCNCC5)c(=O)c4cn3)cc(F)c2n1] |
| 1276 | 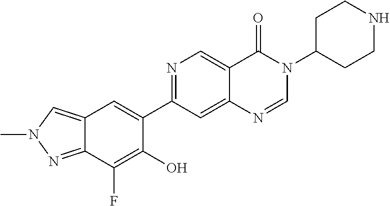 [SMILES: Cn1cc2cc(-c3cc4ncn(C5CCNCC5)c(=O)c4cn3)c(O)c(F)c2n1] |
| 1277 | 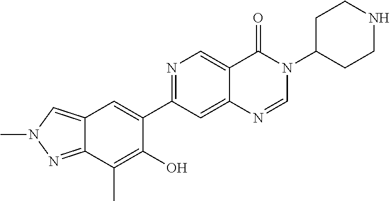 [SMILES: Cc1c(O)c(-c2cc3ncn(C4CCNCC4)c(=O)c3cn2)cc2cn(C)nc12] |
| 1278 | 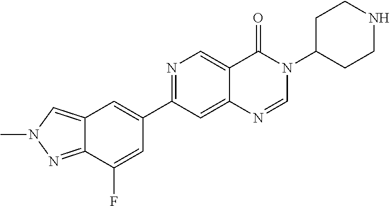 [SMILES: Cn1cc2cc(-c3cc4ncn(C5CCNCC5)c(=O)c4cn3)cc(F)c2n1] |
| 1279 | 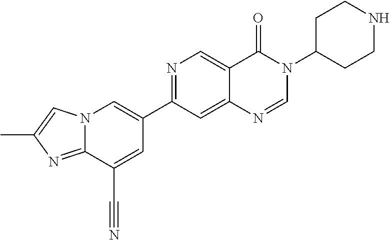 [SMILES: Cc1cn2cc(-c3cc4ncn(C5CCNCC5)c(=O)c4cn3)cc(C#N)c2n1] |
| 1280 | 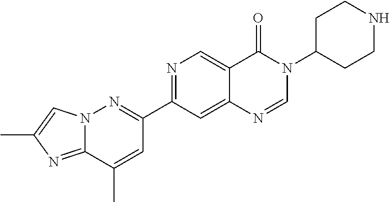 [SMILES: Cc1cn2nc(-c3cc4ncn(C5CCNCC5)c(=O)c4cn3)cc(C)c2n1] |
| 1281 | 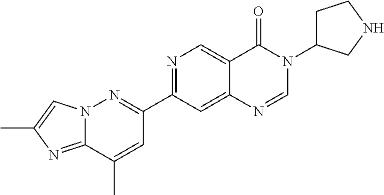 [SMILES: Cc1cn2nc(-c3cc4ncn(C5CCNC5)c(=O)c4cn3)cc(C)c2n1] |
| 1282 | 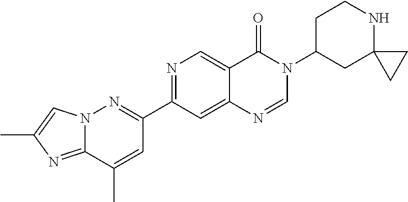 [SMILES: Cc1cn2nc(-c3cc4ncn(C5CCNC6(CC6)C5)c(=O)c4cn3)cc(C)c2n1] |

In some embodiments, the compound of Formula (VIII) is selected from a compound in Table 8.

TABLE 8
Exemplary Compounds of Formula (VIII)

| Cmpd | |
|---|---|
| No. | Structure |
| 2148 | 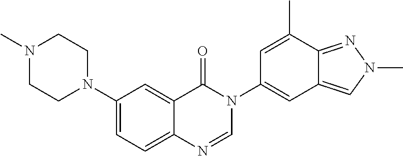 |
| 2149 | 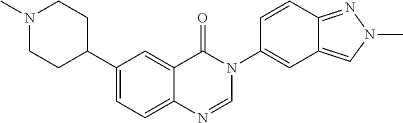 |
| 2150 | 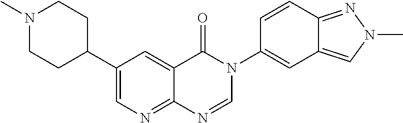 |
| 2151 | 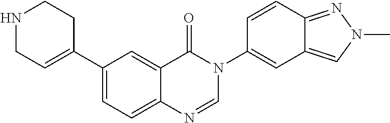 [SMILES: Cn1cc2cc(-n3cnc4ccc(C5=CCNCC5)cc4c3=O)ccc2n1] |
| 2154 | 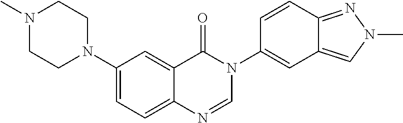 |
| 2155 | 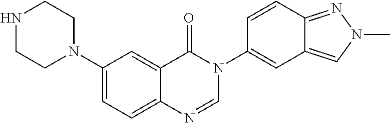 [SMILES: Cn1cc2cc(-n3cnc4ccc(N5CCNCC5)cc4c3=O)ccc2n1] |
| 2164 | 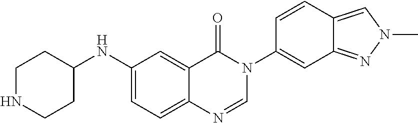 [SMILES: Cn1cc2ccc(-n3cnc4ccc(NC5CCNCC5)cc4c3=O)cc2n1] |
| 2168 | 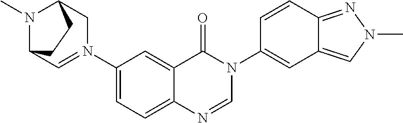 |
| 2169 | 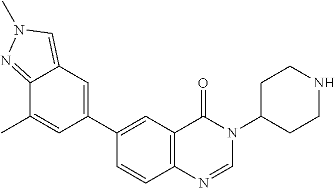 [SMILES: Cc1cc(-c2ccc3ncn(C4CCNCC4)c(=O)c3c2)cc2cn(C)nc12] |
| 2170 | 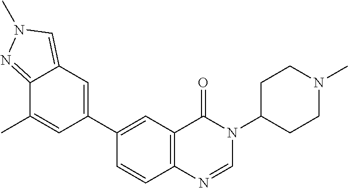 |
| 2171 | 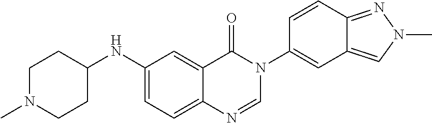 |
| 2184 | 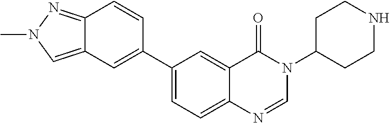 [SMILES: Cn1cc2cc(-c3ccc4ncn(C5CCNCC5)c(=O)c4c3)ccc2n1] |
| 2194 | 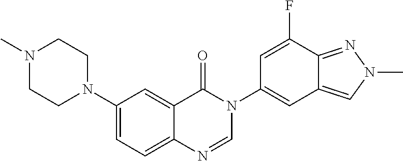 |
| 2195 | 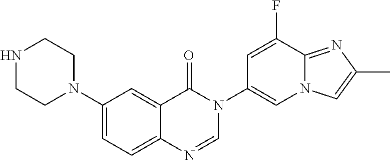 [SMILES: Cc1cn2cc(-n3cnc4ccc(N5CCNCC5)cc4c3=O)cc(F)c2n1] |
| 2196 | 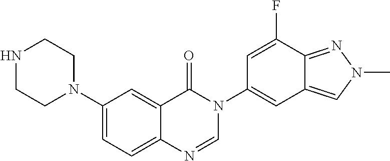 [SMILES: Cn1cc2cc(-n3cnc4ccc(N5CCNCC5)cc4c3=O)cc(F)c2n1] |
| 2197 | 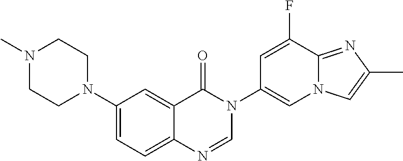 |
| 2198 | 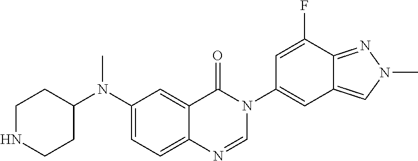 [SMILES: CN(c1ccc2ncn(-c3cc(F)c4nn(C)cc4c3)c(=O)c2c1)C1CCNCC1] |
| 2199 | 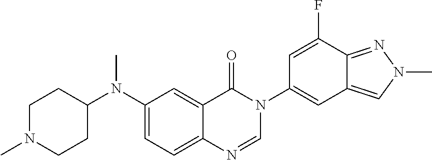 |
| 2200 | 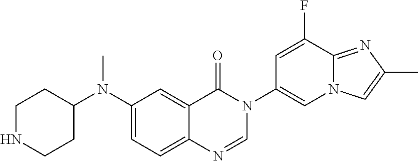 [SMILES: Cc1cn2cc(-n3cnc4ccc(N(C)C5CCNCC5)cc4c3=O)cc(F)c2n1] |
| 2201 | 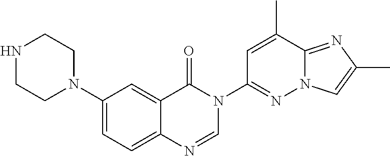 [SMILES: Cc1cn2nc(-n3cnc4ccc(N5CCNCC5)cc4c3=O)cc(C)c2n1] |
| 2202 | 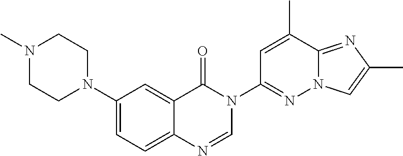 |

In some embodiments, the compound of Formula (IX) is selected from a compound in Table 9.

TABLE 9
Exemplary compounds of Formula (IX)

| Cmpd | |
|---|---|
| No. | Structure |
| 2183 | 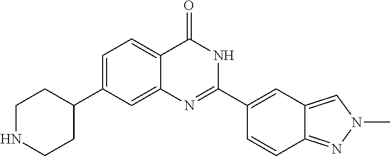 [SMILES: Cn1cc2cc(-c3nc4cc(C5CCNCC5)ccc4c(=O)[nH]3)ccc2n1] |
| 2211 | 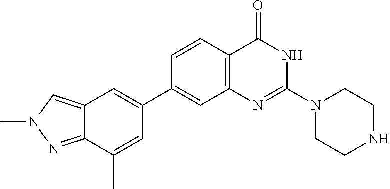 [SMILES: Cc1cc(-c2ccc3c(=O)[nH]c(N4CCNCC4)nc3c2)cc2cn(C)nc12] |
| 2212 | 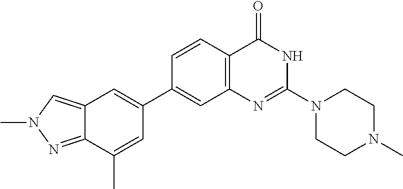 |
| 2213 | 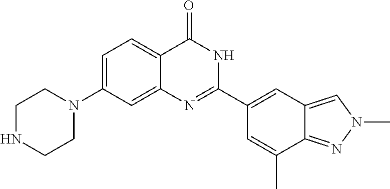 [SMILES: Cc1cc(-c2nc3cc(N4CCNCC4)ccc3c(=O)[nH]2)cc2cn(C)nc12] |
| 2214 | 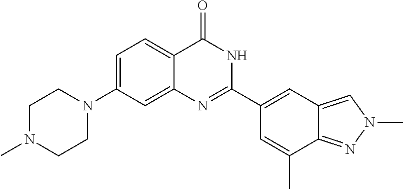 |
| 2215 | 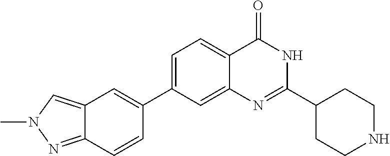 [SMILES: Cn1cc2cc(-c3ccc4c(=O)[nH]c(C5CCNCC5)nc4c3)ccc2n1] |

### Pharmaceutical Compositions, Kits, and Administration

The present invention provides pharmaceutical compositions comprising a compound of Formula (I), (II), (III), (IV), (V), (VI), (VII), (VIII), or (IX), e.g., a compound of Formula (I), (II), (III), (IV), (V), (VI), (VII), (VIII), or (IX) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I), (II), (III), (IV), (V), (VI), (VII), (VIII), or (IX) or a pharmaceutically acceptable salt thereof, and optionally a pharmaceutically acceptable excipient. In certain embodiments, the compound of Formula (I), (II), (III), (IV), (V), (VI), (VII), (VIII), or (IX) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I), (II), (III), (IV), (V), (VI), (VII), (VIII), or (IX) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I), (II), (III), (IV), (V), (VI), (VII), (VIII), or (IX) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I), (II), (III), (IV), (V), (VI), (VII), (VIII), or (IX) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I), (II), (III), (IV), (V), (VI), (VII), (VIII), or (IX) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I), (II), (III), (IV), (V), (VI), (VII), (VIII), or (IX). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I), (II), (III), (IV), (V), (VI), (VII), (VIII), or (IX). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I), (II), (III), (IV), (V), (VI), (VII), (VIII), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAM15, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASH1L-IT1, ASNSD1, ASPM, ATAD5, ATF1, ATG4A, ATG16L2, ATM, ATN1, ATP11C, ATP6V1G3, ATP13A5, ATP7A, ATP7B, ATR, ATXN2, ATXN3, ATXN7, ATXN10, AXIN1, B2 M, B4GALNT3, BBS4, BCL2, BCL2L1, BCL2-like 11 (BIM), BCL11B, BBOX1, BCS1L, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf101, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, C1R, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDKI1B, CD33, CD46, CDH1, CDH23, CDK6, CDKI1B, CDK13, CEBPZ, CEL, CELSR3, CENPA, CENP1, CENPT, CENTB2, CENTG2, CEPJ10, CEP170, CEP192, CETP, CFB, CFTR, CFH, CGN, CGNL1, CHAF1A, CHD9, CHIC2, CHL1, CHN1, CHM, CLEC16A, CL1C2, CLCN1, CLINT1, CLK1, CLPB, CLPTM1, CMIP, CMYA5, CNGA3, CNOT1, CNOT7, CNTN6, COG3, COL11A1, COL11A2, COL12A1, COL14A1, COL15A1, COL17AM, COL19A1, COL1A1, COL1A2, COL2A, COL3A1, COL4A, COL4A2, COL4A5, COL4A6, COL5A2, COL6A1, COL7A, COL9A, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF1, DNAH3, DNAH8, DNA11, DNAJA4, DNAJC13, DNAJC7, DNMT1, DNTTIP2, DOCK4, DOCK5, DOCK10, DOCK11, DOT1L, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBN1, FBX015, FBXO18, FBXO38, FCGBP, FECH, FEZ2, FGA, FGD6, FGFR2, FGFR1OP, FGFR1OP2, FGFR2, FGG, FGR, FIX, FKBP3, FL11, FLJ35848, FLJ36070, FLNA, FN1, FNBP1L, FOLH1, FOSL1, FOSL2, FOXK1, FOAM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GH1, GHR, GHV, GJA1, GLA, GLT8D1, GNA11, GNAQ, GNAS, GNB5, GOLGB1, GOLT1A, GOLT1B, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXC10, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF, IKZF3, IL1R2, IL5RA, IL7RA, IMMT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, 1TGB3, ITGB4, ITIH1, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIF5A, KIF5B, KIF9, KIN, KIR2DL5B, KIR3DL2, KIR3DL3, KIT, KLF3, KLF5, KLF7, KLF10, KLF12, KLF16, KLHL20, KLK12, KLKB1, KMT2A, KMT2B, KPNA5, KRAS, KREMEN1, KRIT1, KRT5, KRTCAP2, KYNU, L1CAM, L3 MBTL, L3 MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAG11, MAGT1, MALT1, MAP2K1, MAP4K4, MAPK8IP3, MAPK9, MAPT, MARC, MARCHS, MATN2, MBD3, MCF2L2, MCM6, MDGA2, MDM4, ASXL1, FUS, SPR54, MECOM, MEF2C, MEF2D, MEGF10, MEGF11, MEMO1, MET, MGA, MGAM, MGAT4A, MGAT5, MGC16169, MGC34774, MKKS, MIB1, MIER2, MITF, MKL2, MLANA, MLH1, MLL5, MLX, MME, MPDZ, MPI, MRAP2, MRPL11, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEKS, NEK11, NF1, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NR1D1, NR1H3, NR1H4, NR4A3, NR5A1, NRXN1, NSMAF, NSMCE2, NT5C, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPA1, OPN4, OPTN, OSBPL11, OSBPL8, OSGEPL1, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PD1A3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3R1, PIP5K1A, PITRM1, PIWIL3, PKD1, PKHD1L1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPFIA2, PPP1R12A, PPP3CB, PPP4C, PPP4RIL, PPP4R2, PRAME, PRC1, PRDM1, PREX1, PREX2, PRIM1, PRIM2, PRKAR1A, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN11, PTPN22, PTPRD, PTPRK, PTPRM, PTPRN2, PTPRT, PUS10, PVRL2, PYGM, QRSL1, RABI1FIP2, RAB23, RAF1, RALBP1, RALGDS, RB1CC1, RBL2, RBM39, RBM45, RBPJ, RBSN, REC8, RELB, RFC4, RFTJ, RFTN1, RHOA, RHPN2, RIF1, RIT1, RLN3, RMND5B, RNF11, RNF32, RNFT1, RNGTT, ROCK1, ROCK2, RORA, RP1, RP6KA3, RP11-265F1, RP13-36C9, RPAP3, RPN1, RPGR, RPL22, RPL22L1, RPS6KA6, RREB1, RRM1, RRP1B, RSK2, RTEL1, RTF1, RUFY1, RUNX1, RUNX2, RXRA, RYR3, SAAL1, SAE1, SALL4, SAT1, SATB2, SBCAD, SCN1A, SCN2A, SCN3A, SCN4A, SCN5A, SCN8A, SCNA, SCN11A, SCO1, SCYL3, SDC1, SDK1, SDK2, SEC24A, SEC24D, SEC31A, SEL1L, SENP3, SENP6, SENP7, SERPINA1, SETD3, SETD4, SETDB1, SEZ6, SFRS12, SGCE, SGOL2, SGPL1, SH2D1A, SH3BGRL2, SH3PXD2A, SH3PXD2B, SH3RF2, SH3TC2, SHOC2, SIPAIL2, SIPAIL3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX, SMS, SMTN, SNCAIP, SNORD86, SNRK, SNRP70, SNX5, SNX6, SOD1, SOD10, SOS, SOS2, SOX5, SOX6, SOX8, SP1, SP2, SP3, SPH110, SPAG9, SPATA13, SPATA4, SPATS1, SPECC1L, SPDEF, SP11, SPINKS, SPP2, SPTA1, SRF, SRM, SRP72, SSX3, SSX5, SSX9, STAG1, STAG2, STAMBPL1, STARD6, STAT1, STAT3, STAT5A, STAT5B, STAT6, STK17B, STX3, STXBP1, SUCLG2, SULF2, SUPT6H, SUPT16H, SV2C, SYCP2, SYT6, SYCP1, SYTL3, SYTL5, TAF2, TARDBP, TBC1D3G, TBC1D8B, TBC1D26, TBC1D29, TBCEL, TBK1, TBP, TBPL1, TBR1, TBX, TCEB3, TCF3, TCF4, TCF7L2, TCFL5, TCF12, TCP11L2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGS1, THAP7, THAP12, THOC2, TIAL1, TIAM2, TIMM50, TLK2, TM4SF20, TM6SF1, TMEM27, TMEM77, TMEM156, TMEM194A, TMF1, TMPRSS6, TNFRSF10A, TNFRSF10B, TNFRSF8, TNK2, TNKS, TNKS2, TOM1L1, TOM1L2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTF1, TTLL5, TTLL9, TTN, TTPAL, TTR, TUSC3, TXNDC10, UBE3A, UCK1, UGT1A1, UHRF1BP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VT11A, VTI1B, VWA3B, WDFY2, WDR16, WDR17, WDR26, WDR44, WDR67, WDTC1, WRN, WRNIP1, WT1, WWC3, XBPJ, XRN1, XRRN2, XX-FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPM1, ZFYVE1, ZFX, ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1CF, A4GALT, AAR2, ABAT, ABCA11P, ZNF721, ABCA5, ABHD10, ABHD13, ABHD2, ABHD6, AC000120.3, KR1T1, AC004076.1, ZNF772, AC004076.9, ZNF772, AC004223.3, RAD51D, AC0004381.6, AC006486.1, ERF, AC0007390.5, AC007780.1, PRKAR1A, AC0007998.2, INO80C, AC0009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDD1A, AC073610.3, ARF3, AC074091.1, GPN1, AC079447.1, LIPT1, AC092587.1, AC079594.2, TRIM59, AC091060.1, C18orf21, AC092143.3, MC1R, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHD1L, ACADM, ACAP3, ACKR2, RP11-141 M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADH1B, ADIPOR1, ADNP, ADPRH, AGBL5, AGPAT1, AGPAT3, AGR2, AGTR1, AHDC1, AH11, AHNAK, AIFM1, AIFM3, AIMP2, AK4, AKAP1, AKNAD1, CLCC1, AKR1A1, AKT1, AKT1S1, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDH1B1, ALDH3A1, ALDH3B2, ALDOA, ALKBH2, ALPL, AMD1, AMICA1, AMN1, AMOTL2, AMY1B, AMY2B, ANAPC10, ANAPC11, ANAPC15, ANG, RNASE4, AL163636.2, ANGEL2, ANGPTL1, ANKMY1, ANKRD11, ANKRD28, ANKRD46, ANKRD9, ANKS3, ANKS3, RP11-127I20.7, ANKS6, ANKZF1, ANPEP, ANXA11, ANXA2, ANXA8L2, AL603965.1, AOC3, AP000304.12, CRYZL1, AP000311.1, CRYZL1, AP000893.2, RAB30, AP001267.5, ATP5 MG, AP002495.2, AP003175.1, OR2AT4, AP003419.1, CLCF1, AP005263.1, ANKRD12, AP006621.5, AP006621.1, AP1G1, AP3 M1, AP3 M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARID1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATP5F1A, ATP5G2, ATP5J, ATP5 MD, ATP5PF, ATP6AP2, ATP6V0B, ATP6V1C1, ATP6V1D, ATP7B, ATXN1, ATXA1L, IST1, ATXA3, ATXA7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCS1L, BDH1, BDKRB2, AL355102.2, BEST1, BEST3, BEX4, BHLHB9, BID, BIN3, BIRC2, BIVM, BIVM-ERCC5, BIVM, BLCAP, BLK, BLOCIS1, RPI1-644F5.10, BLOCIS6, AC090527.2, BLOCIS6, RP11-96020.4, BLVRA, BMF, BOLA1, BORCS8-MEF2B, BORCS8, BRCA1, BRD1, BRDT, BRINP3, BROX, BTBD10, BTBD3, BTBD9, BTD, BTF3L4, BTNL9, BUB1B-PAK6, PAK6, BUB3, C10orf68, C11orf1, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, CID, CIGALT1, CIQB, CIQTNF1, C1S, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CA5BP1, CA5B, CABYR, CALCA, CALCOCO1, CALCOCO2, CALM1, CALM3, CALML4, RP11-315D16.2, CALN1, CALU, CANT1, CANX, CAP1, CAPN12, CAPS2, CARD8, CARHSP1, CARNS1, CASC1, CASP3, CASP7, CBFA2T2, CBS, CBY1, CCBL1, CCBL2, RBMXL1, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB1IP1, CCNC, CCND3, CCNG1, CCP110, CCR9, CCT7, CCT8, CD151, CD1D, CD200, CD22, CD226, CD276, CD36, CD59, CDC26, CDC42, CDC42SE1, CDC42SE2, CDHR3, CDK10, CDK16, CDK4, CDKAL1, CDKL3, CTD-2410N18.4, CDKN1A, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHD1L, CHD8, CHFR, ZNF605, CHIA, CHID1, CHL1, CHM, CHMP1A, CHMP3, RNF103-CHMP3, CHRNA2, CIDEC, CIRBP, CITED1, CKLF-CMTM1, CMTM1, CKMT1B, CLDN12, CTB-13L3.1, CLDND1, AC021660.3, CLDND1, CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COAMD3-BMI1, BMI1, COPS5, COPS7B, COQ8A, CORO6, COTL1, COX14, RP4-60503.4, COX7A2, COX7A2L, COX7B2, CPA4, CPA5, CPEB1, CPNE1, AL109827.1, RBM12, CPNE1, RP1-309K20.6, RBM12, CPNE3, CPSF3L, CPT1C, CREB3L2, CREM, CRP, CRYZ, CS, AC073896.1, CS, RP11-977G19.10, CSAD, CSDE1, CSF2RA, CSGALNACT1, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB, KDELR2, DARS, DBNL, DCAF11, DCAF8, PEX19, DCLRE1C, DCTD, DCTN1, DCTN4, DCUN1D2, DDR1, DDX11, DDX19B, AC012184.2, DDX19B, RP11-529K1.3, DDX25, DDX39B, ATP6V1G2-DDX39B, SNORD84, DDX42, DDX60L, DEDD, DEDD2, DEFA1, DEFA1B, DEFA1B, DEFA3, DENND1C, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKN, DMTF1, DMTN, DNAJC14, DNAJC19, DNAL1, DNASE1L1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RPL1-500 M8.7, DZIP1L, E2F6, ECHDC1, ECSIT, ECT2, EDC3, EDEM1, EDEM2, MMP24-AS1, RP4-61404.11, EEF1AKNMT, EEF1D, EFEMP1, EFHC1, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL1, ELOVL7, ELP1, ELP6, EML3, EMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERLIN2, ERMARD, ERRFI1, ESR2, RP11-544I20.2, ESRRA, ESRRB, ESRRG, ETFA, ETFRF1, ETV1, ETV4, ETV7, EVA1A, EVC2, EVX1, EXD2, EXO5, EXOC1, EXOC2, FAAP24, FABP6, FADS1, FADS2, FAHD2B, FAM107B, FAM111A, FAM111B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANC1, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLR1, FOXP1, FOXK1, FOAM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT11, GALNT14, GALNT6, GAPVD1, GARNL3, GAS2L3, GAS8, GATA1, GATA2, GATA4, GBA, GCNT1, GDPD2, GDPD5, GEMIN7, MARK4, GEMIN8, GGA3, GGACT, AL356966.1, GGPS1, GHRL, GID8, GIGYF2, GIMAP8, GIPC1, GJB1, GJB6, GLB1L, GLI1, GLT8D1, GMFG, GMPR2, GNAI2, GNAQ, GNB1, GNB2, GNE, GNG2, GNGT2, GNPDA1, GNPDA2, GOLGA3, CHFR, GOLGA4, GOLPH3L, GOLT1B, GPBP1L1, GPER1, GPR116, GPR141, EPDR1, GPR155, GPR161, GPR56, GPR63, GPR75-ASB3, ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCY1A3, GUCY1B3, GUK1, GULP1, GYPC, GYS1, GZF1, HAGH, HA02, HAPLN3, HAVCR1, HAX1, HBG2, AC104389.4, HBG2, AC104389.4, HBE1, HBG2, AC104389.4, HBE1, OR51B5, HBG2, HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX, HOXA3, HOXB3, HOXB3, HOXB4, HOXC4, HOXD3, HOXD3, HOXD4, HPCAL1, HPS4, HPS5, HRH1, HS3ST3A1, HSH2D, HSP90AA1, HSPD1, HTT, HUWE1, HYOU1, IAH1, ICA1L, ICAM2, ICE2, ICK, IDH2, IDH3G, IDS, IFI27, IFI44, IFT20, IFT22, IFT88, IGF2, INS-IGF2, IGF2BP3, IGFBP6, IKBKAP, IKBKB, IL11, IL18BP, IL18RAP, IL1RAP, IL1RL1, IL18R1, IL1RN, IL32, IL411, NUP62, AC011452.1, IL411, NUP62, CTC-326K19.6, IL6ST, ILVBL, IMMP1L, IMPDH1, INCA1, ING1, INIP, INPP1, INPP5J, INPP5K, INSIG2, INTS11, INTS12, INTS14, IP6K2, IP6K3, IPO11, LRRC70, IQCE, IQGAP3, IRAK4, IRF3, IRF5, IRF6, ISG20, IST1, ISYNA1, ITFG2, ITGB1BP1, ITGB7, ITIH4, RP5-966 M1.6, ITPRIPL1, JADE1, JAK2, JARID2, JDP2, KANK1, KANK1, RP11-31F19.1, KANK2, KANSL1L, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDM1B, KDM4A, AL451062.3, KHNYN, KIAA0040, KIAA0125, KIAA0196, KIAA0226L, PPP1R2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC1, APOPT1, AL139300.1, KLC4, KLHDC4, KLHDC8A, KLHL13, KLHL18, KLHL2, KLHL24, KLHL7, KLK11, KLK2, KLK5, KLK6, KLK7, KNOP1, KRBA2, AC135178.2, KRBA2, RPII-849F2.7, KRIT1, KRT15, KRT8, KTN1, KXD1, KYAT3, RBMXL1, KYNU, L3 MBTL1, LACC, LARGE, LARP4, LARP7, LAT2, LBHD1, LCA5, LCA5L, LCTL, LEPROTL1, LGALS8, LGALS9C, LGMN, LHFPL2, LIG4, LIMCH1, LIMK2, LIMS2, LINC00921, ZNF263, LIPF, LLGL2, LMAN2L, LMCD1, LMF1, RPII-161 M6.2, LMO1, LMO3, LOXHD1, LPAR1, LPAR2, LPAR4, LPAR5, LPAR6, LPHN1, LPIN2, LPIN3, LPP, LRFN5, LRIF1, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYG1, LYL1, LYPD4, LYPD6B, LYRM1, LYRM5, LYSMD4, MACC1, MAD1L1, MAD1L1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASP1, MATK, MATR3, MATR3, SNHG4, MB, MBD5, MBNL1, MBOAT7, MCC, MCFD2, MCM9, MCOLN3, MCRS1, MDC1, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGF10, MEI1, MEIS2, MELK, MET, METTL13, METTL23, MFF, MFN2, MFSD2A, MGST3, MIB2, MICAL1, MICAL3, MICOS10, NBL1, MICOS10-NBL1, MID1, MINA, MINOS1-NBL1, MINOS1, MIOS, MIPOL1, MIS12, MKLN1, MKNK1, MKNK1, MOB3C, MLF2, MLH1, MMP17, MOBP, MOCS1, MOGS, MOK, MORF4L1, MPC1, MPC2, MPG, MP1, MPP1, MPP2, MPPE1, MPST, MRAS, MRO, MROH1, MROH7-TTC4, MROH7, MRPL14, MRPL24, MRPL33, BABAM2, MRPL33, BRE, MRPL47, MRPL48, MRPL55, MRRF, MRTFA, MRTFB, MRV11, MS4A1, MS4A15, MS4A3, MS4A6E, MS4A7, MS4A14, MSANTD3, MSANTD4, MSH5, MSH5-SAPCD1, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMR10, MTRF1, MTRR, MTUS2, MUTYH, MVK, MX1, MX2, MYH10, MYL12A, MYB, MYD88, MYL5, MYLIP, MYNN, MYO15A, MYO1B, MYOM2, MZF1, N4BP2L2, NAA60, NAB1, NAE1, NAGK, NAP1L1, NAPIL4, NAPG, NARFL, NARG2, NAT1, NAT10, NBPFI1, WI2-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFV1, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOLO, NOL8, NONO, NPAS1, NPIPA8, RPII-1212A22.1, NPIPB3, NPIPB4, NPIPB9, NPL, NPM1, NPPA, NQO2, NR1H3, NR2C2, NR2F2, NR4A1, NRDC, NREP, NRF1, NRG4, NRIP1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF, NXPE1, NXPE3, OARD1, OAT, OAZ2, OCIAD1, OCLN, ODF2, OGDHL, OGFOD2, AC026362.1, OGFOD2, RPII-197N18.2, OLA1, OPRL1, OPTN, OR2H1, ORAI2, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARP11, PARVG, PASK, PAX6, PBRM1, PBX1P1, PCBP3, PCBP4, AC115284.1, PCBP4, RPI1-155D18.14, RPI1-155D18.12, PCGF3, PCGF5, PCNP, PCSK9, PDCD10, PDCD6, AHRR, PDDC1, PDGFRB, PDIA6, PDIK1L, PDLIM7, PDP1, PDPK1, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4 KB, PIAS2, PICALM, PIF1, PIGN, PIGO, PIGT, PIK3CD, PILRB, STAG3L5P-PVRIG2P-PILRB, PIP5K1B, PIR, PISD, PIWIL4, FUT4, PKD2, PKIA, PKIG, PKM, PKN2, PLA1A, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP, AKTIS1, PNMT, PNPLA4, PNPLA8, PNPO, PNRC1, POCIB, POFUT1, POLB, POLD1, POLH, POL1, POLL, POLR1B, POM121, POM121C, AC006014.7, POM121C, AC211429.1, POMC, POMT1, POP1, PORCN, POU5F1, PSORS1C3, PPARD, PPARG, PPHLN1, PPIL3, PPIL4, PPM1A, PPM1B, AC013717.1, PPP1CB, PPP1R11, PPP1R13L, PPP1R26, PPP1R9A, PPP2R2B, PPP3CA, PPP6R1, PPP6R3, PPT2, PPT2-EGFL8, EGFL8, PPWDJ, PRDM2, PRDM8, PRELID3A, PREPL, PRICKLE1, PRKAG1, PRMT2, PRMT5, PRMT7, PROM1, PRPS1, PRPSAP2, PRR14L, PRR15L, PRR5, PRR5-ARHGAP8, PRR5L, PRR7, PRRC2B, PRRT4, PRSS50, PRSS45, PRSS44, PRUNE, PRUNE1, PSEN1, PSMA2, PSMF, PSORS1C1, PSPH, PSRC1, PTBP3, PTHLH, PTK2, PTPDC1, PTPRM, PUF60, PUM2, PUS1, PUS10, PXN, PXYLP1, PYCR1, QRICH1, R3HCC1L, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAP1, RAD17, RAD51L3-RFFL, RAD51D, RAD52, RAE1, RAI14, RAI2, RALBP1, RAN, RANGAP1, RAP1A, RAP1B, RAP1GAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7, AP002373.1, RBM7, RPI1-212D19.4, RBMS2, RBMY1E, RBPJ, RBPMS, RBSN, RCBTB2, RCC1, RCC1, SNHG3, RCCD1, RECQL, RELL2, REPIN1, AC073111.3, REPIN1, ZNF775, RER1, RERE, RFWD3, RFX3, RGL2, RGMB, RGS11, RGS3, RGS5, AL592435.1, RHBDD1, RHNO1, TULP3, RHOC, AL603832.3, RHOC, RP11-426L16.10, RHOH, RIC8B, RIMKLB, RIN1, RIPK2, RIT1, RLIM, RNASE4, ANG, AL163636.6, RNASEK, RNASEK-C17orf49, RNFI11, RNF123, RNF13, RNF14, RNF185, RNF216, RNF24, RNF32, RNF34, RNF38, RNF4, RNF44, RNH1, RNMT, RNPS1, R060, ROPN1, ROPNIB, ROR2, RPI-102H19.8, C6orf163, RPI-283E3.8, CDK11A, RP11-120 M18.2, PRKAR1A, RPII-133K10.2, PAK6, RP11-164J13.1, CAPN3, RP11-21J18.1, ANKRD12, RP11-322E10.6, INO80C, RP11-337C18.10, CHD1L, RPI1-432B6.3, TRIM59, RPI1-468E2.4, IRF9, RP11-484 M3.5, UPK1B, RP11-517H2.6, CCR6, RP11-613 MI0.9, SLC25A51, RP11-659G9.3, RAB30, RP11-691N7.6, CTNND1, RP11-849H4.2, RP11-896JI0.3, NKX2-1, RPI1-96020.4, SQRDL, RP11-986E7.7, SERPINA3, RP4-769N13.6, GPRASP1, RP4-769N13.6, GPRASP2, RP4-798P15.3, SEC16B, RP5-1021I20.4, ZNF410, RP6-109B7.3, FLJ27365, RPE, RPH3AL, RPL15, RPL17, RPL17-C18orf32, RPL17, RPL23A, RPL36, HSD11B1L, RPP38, RPS20, RPS27A, RPS3A, RPS6KA3, RPS6KC1, RPS6KL1, RPUSD1, RRAGD, RRAS2, RRBP1, RSL1DJ, RSRC2, RSRP1, RUBCNL, RUNX1T1, RUVBL2, RWDD1, RWDD4, S100A13, AL162258.1, S100A13, RP1-178F15.5, S100A16, S100A4, S100A3, S100A6, S100PBP, SAA1, SACM1L, SAMD4B, SAR1A, SARAF, SARNP, RP11-762I7.5, SCAMPS, SCAP, SCAPER, SCFD1, SCGB3A2, SCIN, SCML1, SCNN1D, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-Sep, 2-Sep, SERGEF, AC055860.1, SERP1, SERPINA1, SERPINA5, SERPINB6, SERPING1, SERPINH1, SERTAD3, SETD5, SFMBT1, AC096887.1, SFTPA1, SFTPA2, SFXN2, SGCD, SGCE, SGK3, SGK3, C8orf44, SH2B1, SH2D6, SH3BP1, Z83844.3, SH3BP2, SH3BP5, SH3D19, SH3YL1, SHC1, SHISA5, SHMT1, SHMT2, SHOC2, SHROOM1, SIGLEC5, SIGLEC14, SIL1, SIN3A, SIRT2, SIRT6, SKP1, STAT4, AC104109.3, SLAIN1, SLCI0A3, SLC12A9, SLC14A1, SLC16A6, SLC1A2, SLC1A6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCO1A2, SLCO1C1, SLCO2B1, SLFN11, SLFN12, SLFNL1, SLMO1, SL™, SLU7, SMAD2, SMAP2, SMARCA2, SMARCE1, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPD1, SNRPD2, SNRPN, SNRPN, SNURF, SNUPN, SNX11, SNX16, SNX17, SOAT1, SOHLH2, CCDC169-SOHLH2, CCDC169, SORBS1, SORBS2, SOX5, SP2, SPART, SPATA20, SPATA21, SPATS2, SPATS2L, SPDYE2, SPECC1, SPECC1L, SPECC1L-ADORA2A, SPECCIL-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF1l, SSX1, SSX2IP, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RPII-463D19.2, STEAP2, STEAP3, STIL, STK25, STK33, STK38L, STK40, STMN1, STON1, STONI-GTF2AIL, STRAP, STRBP, STRC, AC011330.5, STRC, CATSPER2, STRC, CATSPER2, AC011330.5, STRC, STRCP1, STT3A, STX16-NPEPL1, NPEPL1, STX5, STX6, STX8, STXBP6, STYK1, SULT1A1, SULT1A2, SUMF2, SUN1, SUN2, SUN2, DNAL4, SUOX, SUPT6H, SUV39H2, SV2B, SYBU, SYNCRIP, SYNJ2, SYT1, SYTL4, TAB2, TACC1, TADA2B, TAF1C, TAF6, AC073842.2, TAF6, RPII-506 M12.1, TAF9, TAGLN, TANK, TAPSAR1, PSMB9, TAPT1, TATDN1, TAZ, TBC1D1, TBC1D12, HELLS, TBC1D15, TBC1D3H, TBC1D3G, TBC1D5, TBC1D5, SATB1, TBCA, TBCEL, TBCEL, AP000646.1, TBL1XR1, TBP, TBX5, TBXAS1, TCAF1, TCEA2, TCEAL4, TCEAL8, TCEAL9, TCEANC, TCEB1, TCF19, TCF25, TCF4, TCP1, TCP10L, AP000275.65, TCP11, TCP11L2, TCTN1, TDG, TDP1, TDRD7, TEAD2, TECR, TENC1, TENT4A, TEX264, TEX30, TEX37, TFDP1, TFDP2, TFEB, TFG, TFP1, TF, TFP1, TGIF1, THAP6, THBS3, THOC5, THRAP3, THUMPD3, TIAL1, TIMM9, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSS11D, TMPRSS5, TMSB15B, TMTC4, TMUB2, TMX2-CTNND1, RPII-691N7.6, CTNND1, TNFAIP2, TNFAIP8L2, SCNM1, TNFRSF10C, TNFRSF9, TNFRSF8, TNFSF12-TNFSF3, TNFSF2, TNFSF3, TNFSF12-TNFSF3, TNFSF3, TNIP1, TNK2, TNNT1, TNRC18, TNS3, TOB2, TOM1L1, TOP1 MT, TOP3B, TOX2, TP53, RP11-199F1.2, TP5311, TP53INP2, TPCN, TPM3P9, AC022137.3, TPT1, TRA2B, TRAF2, TRAF3, TRAPPC12, TRAPPC3, TREH, TREX, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMTIOB, TRMT2B, TRMT2B-AS1, TRNT1, TRO, TROVE2, TRPS1, TRPT1, TSC2, TSGA10, TSPAN14, TSPAN3, TSPAN4, TSPAN5, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD1, TYK2, U2AF1, UBA2, UBA52, UBAP2, UBE2D2, UBE2D3, UBE2E3, UBE21, UBE2J2, UBE3A, UBL7, UBXN11, UBXN7, UGDH, UGGT1, UGP2, UMAD1, AC007161.3, UNC45A, UQCC1, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VILL, VIPR1, VPS29, VPS37C, VPS8, VPS9D1, VRK2, VWA1, VWA5A, WARS, WASF1, WASHC5, WBP5, WDHD1, WDPCP, WDR37, WDR53, WDR6, WDR72, WDR74, WDR81, WDR86, WDYHV1, WFDC3, WHSC1, WIPF1, WSCD2, WWP2, XAGE1A, XAGE1B, XKR9, XPNPEP1, XRCC3, XRN2, XXYLT1, YIF1A, YIF1B, YIPF1, YIPF5, YPEL5, YWHAB, YWHAZ, YYJAP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFANDS, ZFAND6, ZFP1, ZFP62, ZFX, ZFYVE16, ZFYVE19, ZFYVE20, ZFYVE27, ZHX2, AC016405.1, ZHX3, ZIK1, ZIM2, PEG3, ZKSCAN1, ZKSCAN3, ZKSCAN8, ZMAT3, ZMAT5, ZMIZ2, ZMYM6, ZMYND11, ZNF10, AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC0008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158I13.2, ZNF512B, ZNF512B, SAMD10, ZNF521, ZNF532, ZNF544, AC020915.5, ZNF544, CTD-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOXM1 gene.

Exemplary genes that may be modulated by the compounds of Formula (I), (II), (III), (IV), (V), (VI), (VII), (VIII), or (IX) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgcgaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucagcg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugagcg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugcguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuauu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacuge (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugagcg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugaguc (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgcgccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacage (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguagcug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugagcg (SEQ ID NO: 957), AUGgugagcu (SEQ ID NO: 958), AUGgugaggc (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugcgau (SEQ ID NO: 966), AUGgugcgug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguuaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgcgccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacccg (SEQ ID NO: 1173), CAGguacccu (SEQ ID NO: 1174), CAGguaccgc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacugc (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucagcg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccugc (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugcg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugagcg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugcgcc (SEQ ID NO: 1376), CAGgugcgcg (SEQ ID NO: 1377), CAGgugcgga (SEQ ID NO: 1378), CAGgugcggu (SEQ ID NO: 1379), CAGgugcgua (SEQ ID NO: 1380), CAGgugcguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcugc (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggcca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucccg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugcgua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CCAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugcgcg (SEQ ID NO: 1589), CCGgugggcg (SEQ ID NO: 1590), CCGguugguc (SEQ ID NO: 1591), CCUguaaaug (SEQ ID NO: 1592), CCUguaaauu (SEQ ID NO: 1593), CCUguaagaa (SEQ ID NO: 1594), CCUguaagac (SEQ ID NO: 1595), CCUguaagag (SEQ ID NO: 1596), CCUguaagca (SEQ ID NO: 1597), CCUguaagcg (SEQ ID NO: 1598), CCUguaagga (SEQ ID NO: 1599), CCUguaaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugagaa (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguaagag (SEQ ID NO: 1622), CGGgcaggca (SEQ ID NO: 1623), CGGguaagcc (SEQ ID NO: 1624), CGGguaagcu (SEQ ID NO: 1625), CGGguaaguu (SEQ ID NO: 1626), CGGguaauuc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggcc (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugccuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaaug (SEQ ID NO: 1661), CUAguaagcg (SEQ ID NO: 1662), CUAguaagcu (SEQ ID NO: 1663), CUAguaagua (SEQ ID NO: 1664), CUAguaaguc (SEQ ID NO: 1665), CUAguaagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CUGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGgucccgc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugcgug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacagc (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguaccga (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GAGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauucc (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucaggc (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugagcg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugcggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugagcg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugcgua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GUAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaagcg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagege (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugaggc (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UACguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UCCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccueg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagcc (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacggc (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UGGgccccgg (SEQ ID NO: 2820), UGGguaaaau (SEQ ID NO: 2821), UGGguaaagc (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguaagaa (SEQ ID NO: 2832), UGGguaagac (SEQ ID NO: 2833), UGGguaagag (SEQ ID NO: 2834), UGGguaagau (SEQ ID NO: 2835), UGGguaagca (SEQ ID NO: 2836), UGGguaagcc (SEQ ID NO: 2837), UGGguaagcu (SEQ ID NO: 2838), UGGguaaggg (SEQ ID NO: 2839), UGGguaaggu (SEQ ID NO: 2840), UGGguaagua (SEQ ID NO: 2841), UGGguaaguc (SEQ ID NO: 2842), UGGguaagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggeg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054).

Additional exemplary gene sequences and splice site sequences (e.g., 5′ splice site sequences) include AAGgcaagau (SEQ ID NO: 96), AUGguaugug (SEQ ID NO: 937), GGGgugaggc (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgcgagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GAGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGgugguge (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggcgc (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgcgggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugagge (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugagcg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgcgcgcg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugagcg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugegug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgegguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugcgug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggcg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacacc (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugagcg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgcgggaa (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguagugc (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucuggc (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugcguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugcgga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgcggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugcg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugagcg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgccgguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugcguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaagc (SEQ ID NO: 3523), CUGguaaaag (SEQ ID NO: 3524), CCGgugcgga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucagcg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaagcg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaagcg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugcggc (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaagcg (SEQ ID NO: 3684), AAGguccgcg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggcg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacucg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugcgcg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugagcg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGguggugc (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccuge (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaagcg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugcggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugaggc (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaacc (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugcgua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgcgagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugaugc (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGguguggc (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacugc (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggcg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucguce (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugagge (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGguccgcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucucgc (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugcg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugcguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgcccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugcggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggcg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggccc (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagagc (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugcgcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaaauu (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugagcg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugagcg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugaggc (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggcg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550). Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaagcg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugeguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugcggc (SEQ ID NO: 4673), AGGguacuga (SEQ ID NO: 4674), AGAguaagcg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggcgg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUguccgcc (SEQ ID NO: 4836), AAGgugcgga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugeguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgcagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugcgcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugcguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggagc (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugagcg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgcauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugcgcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugcgcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuauuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I), (II), (III), (IV), (V), (VI), (VII), (VIII), or (IX) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C₁/C₂. Human genes encoding hnRNPs include HNRNPA0, HNRNPA1, HNRNPA1L1, HNRNPA1L2, HNRNPA3, HNRNPA2B1, HNRNPAB1, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I), (II), (III), (IV), (V), (VI), (VII), (VIII), or (IX) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I), (II), (III), (IV), (V), (VI), (VII), (VIII), or (IX) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7% 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I), (II), (III), (IV), (V), (VI), (VII), (VIII), or (IX) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I), (II), (III), (IV), (V), (VI), (VII), (VIII), or (IX) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I), (II), (III), (IV), (V), (VI), (VII), (VIII), or (IX). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I), (II), (III), (IV), (V), (VI), (VII), (VIII), or (IX) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I), (II), (III), (IV), (V), (VI), (VII), (VIII), or (IX) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I), (II), (III), (IV), (V), (VI), (VII), (VIII), or (IX) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I), (II), (III), (IV), (V), (VI), (VII), (VIII), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I), (II), (III), (IV), (V), (VI), (VII), (VIII), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I), (II), (III), (IV), (V), (VI), (VII), (VIII), or (IX) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I), (II), (III), (IV), (V), (VI), (VII), (VIII), or (IX) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MDS).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I), (II), (III), (IV), (V), (VI), (VII), (VIII), or (IX) or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I), (II), (III), (IV), (V), (VI), (VII), (VIII), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), pseudobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I), (II), (III), (IV), (V), (VI), (VII), (VIII), or (IX) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashimoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitis interstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I), (II), (III), (IV), (V), (VI), (VII), (VIII), or (IX) or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I), (II), (III), (IV), (V), (VI), (VII), (VIII), or (IX) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I), (II), (III), (IV), (V), (VI), (VII), (VIII), or (IX) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I), (II), (III), (IV), (V), (VI), (VII), (VIII), or (IX) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I), (II), (III), (IV), (V), (VI), (VII), (VIII), or (IX) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I), (II), (III), (IV), (V), (VI), (VII), (VIII), or (IX) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I), (II), (III), (IV), (V), (VI), (VII), (VIII), or (IX) increases expression of the haploinsufficient gene locus. In an embodiment, a compound of Formula (I), (II), (III), (IV), (V), (VI), (VII), (VIII), or (IX) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I), (II), (III), (IV), (V), (VI), (VII), (VIII), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I), (II), (III), (IV), (V), (VI), (VII), (VIII), or (IX) may increase the expression of a target (e.g., a gene) related to an autosomal recessive disease with residual function. Exemplary autosomal recessive diseases with residual function include Friedreich's ataxia, Stargardt disease, Usher syndrome, chloridemia, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I), (II), (III), (IV), (V), (VI), (VII), (VIII), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I), (II), (III), (IV), (V), (VI), (VII), (VIII), or (IX) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I), (II), (III), (IV), (V), (VI), (VII), (VIII), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I), (II), (III), (IV), (V), (VI), (VII), (VIII), or (IX) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I), (II), (III), (IV), (V), (VI), (VII), (VIII), or (IX) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I), (II), (III), (IV), (V), (VI), (VII), (VIII), or (IX) or a pharmaceutically acceptable salt thereof, e.g., as described herein, has a cell permeability measurement (Papp) of <2×10⁻⁶ cm s⁻¹. In an embodiment, a compound of Formula (I), (II), (III), (IV), (V), (VI), (VII), (VIII), or (IX) or a pharmaceutically acceptable salt thereof, e.g., as described herein, has a cell permeability measurement (Papp) of between 2-6×10⁻⁶ cm s⁻¹. In an embodiment, a compound of Formula (I), (II), (III), (IV), (V), (VI), (VII), (VIII), or (IX) or a pharmaceutically acceptable salt thereof, e.g., as described herein, has a cell permeability measurement (Papp) of Papp greater than 6×10⁻⁶ cm s⁻¹. In an embodiment, a compound of Formula (I), (II), (III), (IV), (V), (VI), (VII), (VIII), or (IX) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), (III), (IV), (V), (VI), (VII), (VIII), or (IX) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I), (II), (III), (IV), (V), (VI), (VII), (VIII), or (IX) or a pharmaceutically acceptable salt thereof, e.g., as described herein, has a cell efflux ratio of less than 1.5. In an embodiment, a compound of Formula (I), (II), (III), (IV), (V), (VI), (VII), (VIII), or (IX) or a pharmaceutically acceptable salt thereof, e.g., as described herein, has a cell efflux ratio of between 1.5 and 5. In an embodiment, a compound of Formula (I), (II), (III), (IV), (V), (VI), (VII), (VIII), or (IX) or a pharmaceutically acceptable salt thereof, e.g., as described herein, has a cell efflux ratio greater than 5. In an embodiment, a compound of Formula (I), (II), (III), (IV), (V), (VI), (VII), (VIII), or (IX) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), (III), (IV), (V), (VI), (VII), (VIII), or (IX) or a pharmaceutically acceptable salt thereof, e.g., as described herein, modulates the expression of a target protein (e.g., HTT or MYB) in a reference cell or sample. In an embodiment, a compound of Formula (I), (II), (III), (IV), (V), (VI), (VII), (VIII), or (IX) or a pharmaceutically acceptable salt thereof, e.g., as described herein, increases the expression of a target protein (e.g., HTT or MYB) in a reference cell or sample. In an embodiment, a compound of Formula (I), (II), (III), (IV), (V), (VI), (VII), (VIII), or (IX) or a pharmaceutically acceptable salt thereof, e.g., as described herein, decreases the expression of a target protein (e.g., HTT or MYB) in a reference cell or sample. The effect of an exemplary compound of Formula (I), (II), (III), (IV), (V), (VI), (VII), (VIII), or (IX) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I), (II), (III), (IV), (V), (VI), (VII), (VIII), or (IX) or a pharmaceutically acceptable salt thereof, e.g., as described herein, has a protein abundance response less than 100 nM. In an embodiment, a compound of Formula (I), (II), (III), (IV), (V), (VI), (VII), (VIII), or (IX) or a pharmaceutically acceptable salt thereof, e.g., as described herein, has a protein abundance response between 100-1000 nM. In an embodiment, a compound of Formula (I), (II), (III), (IV), (V), (VI), (VII), (VIII), or (IX) or a pharmaceutically acceptable salt thereof, e.g., as described herein, has a protein abundance response greater than 1000 nM. In an embodiment, a compound of Formula (I), (II), (III), (IV), (V), (VI), (VII), (VIII), or (IX) or a pharmaceutically acceptable salt thereof, e.g., as described herein, has a protein abundance response greater than 10 uM. In an embodiment, a compound of Formula (I), (II), (III), (IV), (V), (VI), (VII), (VIII), or (IX) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), (III), (IV), (V), (VI), (VII), (VIII), or (IX) or a pharmaceutically acceptable salt thereof, e.g., as described herein, modulates the viability of a target cell in a subject or sample. In an embodiment, a compound of Formula (I), (II), (III), (IV), (V), (VI), (VII), (VIII), or (IX) or a pharmaceutically acceptable salt thereof, e.g., as described herein, increases the viability of a target cell in a subject or sample. In an embodiment, a compound of Formula (I), (II), (III), (IV), (V), (VI), (VII), (VIII), or (IX) or a pharmaceutically acceptable salt thereof, e.g., as described herein, decreases the viability of a target cell in a subject or sample. In an embodiment, a compound of Formula (I), (II), (III), (IV), (V), (VI), (VII), (VIII), or (IX) or a pharmaceutically acceptable salt thereof, e.g., as described herein, does not impact the viability of a cell (e.g., is non-toxic) in a subject or sample. The effect an exemplary compound of Formula (I), (II), (III), (IV), (V), (VI), (VII), (VIII), or (IX) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I), (II), (III), (IV), (V), (VI), (VII), (VIII), or (IX) or a pharmaceutically acceptable salt thereof, e.g., as described herein, is tolerated by a target cell at a concentration of less than 100 nM. In an embodiment, a compound of Formula (I), (II), (III), (IV), (V), (VI), (VII), (VIII), or (IX) or a pharmaceutically acceptable salt thereof, e.g., as described herein, is tolerated by a target cell at a concentration of between 100-1000 nM. In an embodiment, a compound of Formula (I), (II), (III), (IV), (V), (VI), (VII), (VIII), or (IX) or a pharmaceutically acceptable salt thereof, e.g., as described herein, is tolerated by a target cell at a concentration of greater than 1000 nM. In an embodiment, a compound of Formula (I), (II), (III), (IV), (V), (VI), (VII), (VIII), or (IX) or a pharmaceutically acceptable salt thereof, e.g., as described herein, is tolerated by a target cell at a concentration of greater than 10 uM.

In some embodiments, a compound of Formula (I), (II), (III), (IV), (V), (VI), (VII), (VIII), or (IX) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,brain}) may be defined as the ratio of unbound brain-free compound concentration to unbound plasma concentration. It is calculated using the following equation:

$${K}_{p , uu , brain} = \frac{{f}_{u , brain} \times {C}_{brain}}{{f}_{u , plasma} \times {C}_{plasma}}$$

C_{brain} and Cₚₗₐₛₘₐ represent the total concentrations in brain and plasma, respectively. In this assay, the f_{u,brain} and C_{fu,plasma} may be the unbound fraction of the compound in brain and plasma, respectively. Both f_{u,brain} and C_{fu,plasma} may be determined in vitro via equilibrium dialysis. In an embodiment, a compound of Formula (I), (II), (III), (IV), (V), (VI), (VII), (VIII), or (IX) or a pharmaceutically acceptable salt thereof, e.g., as described herein, has a Kp value of greater than 5. In an embodiment, a compound of Formula (I), (II), (III), (IV), (V), (VI), (VII), (VIII), or (IX) or a pharmaceutically acceptable salt thereof, e.g., as described herein, has a Kp value between 1 and 5. In an embodiment, a compound of Formula (I), (II), (III), (IV), (V), (VI), (VII), (VIII), or (IX) or a pharmaceutically acceptable salt thereof, e.g., as described herein, has a Kp value between 0.2-1. In an embodiment, a compound of Formula (I), (II), (III), (IV), (V), (VI), (VII), (VIII), or (IX) or a pharmaceutically acceptable salt thereof, e.g., as described herein, has a Kp value of less than 0.2. In an embodiment, a compound of Formula (I), (II), (III), (IV), (V), (VI), (VII), (VIII), or (IX) or a pharmaceutically acceptable salt thereof, e.g., as described herein, has a Kpuu value of greater than 2.5. In an embodiment, a compound of Formula (I), (II), (III), (IV), (V), (VI), (VII), (VIII), or (IX) or a pharmaceutically acceptable salt thereof, e.g., as described herein, has a Kpuu value between 0.5-2.5. In an embodiment, a compound of Formula (I), (II), (III), (IV), (V), (VI), (VII), (VIII), or (IX) or a pharmaceutically acceptable salt thereof, e.g., as described herein, has a Kpuu value between 0.1-0.5. In an embodiment, a compound of Formula (I), (II), (III), (IV), (V), (VI), (VII), (VIII), or (IX) or a pharmaceutically acceptable salt thereof, e.g., as described herein, has a Kpuu value of less than 0.1. In an embodiment, a compound of Formula (I), (II), (III), (IV), (V), (VI), (VII), (VIII), or (IX) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), (III), (IV), (V), (VI), (VII), (VIII), or (IX) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I), (II), (III), (IV), (V), (VI), (VII), (VIII), or (IX) or a pharmaceutically acceptable salt thereof, e.g., as described herein, exhibits selectivity for HTT, e.g., an HTT-related nucleic acid sequence. In an embodiment, a compound of Formula (I), (II), (III), (IV), (V), (VI), (VII), (VIII), or (IX) or a pharmaceutically acceptable salt thereof, e.g., as described herein, exhibits selectivity for SMN2, e.g., an SMN2-related nucleic acid sequence. In an embodiment, a compound of Formula (I), (II), (III), (IV), (V), (VI), (VII), (VIII), or (IX) or a pharmaceutically acceptable salt thereof, e.g., as described herein, exhibits selectivity for Target C, e.g., a Target C-related nucleic acid sequence. In an embodiment, a compound of Formula (I), (II), (III), (IV), (V), (VI), (VII), (VIII), or (IX) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I), (II), (III), (IV), (V), (VI), (VII), (VIII), or (IX) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I), (II), (III), (IV), (V), (VI), (VII), (VIII), or (IX) or a pharmaceutically acceptable salt thereof, e.g., as described herein, has a 3-fold greater selectivity for HTT over MYB. In an embodiment, a compound of Formula (I), (II), (III), (IV), (V), (VI), (VII), (VIII), or (IX) or a pharmaceutically acceptable salt thereof, e.g., as described herein, has a 3-fold greater selectivity for MYB over HTT. In an embodiment, a compound of Formula (I), (II), (III), (IV), (V), (VI), (VII), (VIII), or (IX) or a pharmaceutically acceptable salt thereof, e.g., as described herein, has a 10-fold greater selectivity for HTT over MYB. In an embodiment, a compound of Formula (I), (II), (III), (IV), (V), (VI), (VII), (VIII), or (IX) or a pharmaceutically acceptable salt thereof, e.g., as described herein, has a 10-fold greater selectivity for MYB over HTT. In an embodiment, a compound of Formula (I), (II), (III), (IV), (V), (VI), (VII), (VIII), or (IX) or a pharmaceutically acceptable salt thereof, e.g., as described herein, has a 3-fold greater selectivity for HTT over SMN2. In an embodiment, a compound of Formula (I), (II), (III), (IV), (V), (VI), (VII), (VIII), or (IX) or a pharmaceutically acceptable salt thereof, e.g., as described herein, has a 3-fold greater selectivity for SMN2 over HTT. In an embodiment, a compound of Formula (I), (II), (III), (IV), (V), (VI), (VII), (VIII), or (IX) or a pharmaceutically acceptable salt thereof, e.g., as described herein, has a 10-fold greater selectivity for HTT over SMN2. In an embodiment, a compound of Formula (I), (II), (III), (IV), (V), (VI), (VII), (VIII), or (IX) or a pharmaceutically acceptable salt thereof, e.g., as described herein, has a 10-fold greater selectivity for SMN2 over HTT. In an embodiment, a compound of Formula (I), (II), (III), (IV), (V), (VI), (VII), (VIII), or (IX) or a pharmaceutically acceptable salt thereof, e.g., as described herein, has a 3-fold greater selectivity for MYB over SMN2. In an embodiment, a compound of Formula (I), (II), (III), (IV), (V), (VI), (VII), (VIII), or (IX) or a pharmaceutically acceptable salt thereof, e.g., as described herein, has a 3-fold greater selectivity for SMN2 over MYB. In an embodiment, a compound of Formula (I), (II), (III), (IV), (V), (VI), (VII), (VIII), or (IX) or a pharmaceutically acceptable salt thereof, e.g., as described herein, has a 10-fold greater selectivity for MYB over SMN2. In an embodiment, a compound of Formula (I), (II), (III), (IV), (V), (VI), (VII), (VIII), or (IX) or a pharmaceutically acceptable salt thereof, e.g., as described herein, has a 10-fold greater selectivity for SMN2 over MYB. In an embodiment, a compound of Formula (I), (II), (III), (IV), (V), (VI), (VII), (VIII), or (IX) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I), (II), (III), (IV), (V), (VI), (VII), (VIII), or (IX), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (δ) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 μm, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH-C₁₈ (C18, Ø4.6×50 mm, 3 μm, 120 Å, 40° C.) operating in ESI(+) ionization mode; flow rate=1.2 mL/min. Mobile phase A: Water/5 mM NH₄HCO₃, Mobile phase B: CH₃CN.)

Analytical chiral HPLC: Analytical chiral HPLC was performed on a Agilent 1260 using column: CHIRALPAK IG-3, CHIRALPAK IC-3 or CHIRALPAK OJ-3, with flow rate=1.2 mL/min. Mobile phase=MTBE(DEA):EtOH=50:50).

Preparative HPLC purification: prep-HPLC purification was performed on a Waters-2545 or Shimadzu, using column: X-Select CSH C18 OBD (130 Å, 5 μm, 30 mm×150 mm), XBridge Prep OBD C18 (30×150 mm, 5 μm), XBridge Prep C18 OBD (Sum, 19 mm×150 mm), or YMC-Actus Triart C₁₈ (30×150 mm, 5 μm).

Condition 1: Column: YMC-Actus Triart C18, 30×150 mm, 5 μm; Mobile Phase A: water (10 mmol/L NH₄HCO₃), Mobile Phase B: ACN; Flow rate: 60 mL/min; Gradient 1:10% B up to 40% B in 8 min; Gradient 2: 5% B to 55% in 8 min Gradient 3: 10% B to 28% B in 10 min; Gradient 4: 5% B to 50% B in 8 min; Gradient 5: 10% B to 65% B in 8 min; Gradient 6: 45% B to 67% B in 8 min; Gradient 7: 5% B to 70% B in 8 min; Gradient 8: 5% B to 40% B in 8 min.

Condition 2: Column: XBridge Prep OBD C18 Column, 30×150 mm, 5 μm; Mobile Phase A: Water(10 mmol/L NH₄HCO₃); Mobile Phase B: ACN; Flow rate: 60 mL/min; Gradient 1: 5% B to 30% B in 10 min; Gradient 2: 5% B to 33% B in 8 min; Gradient 3: 3% B to 33% B in 6 min; Gradient 4: 50% B to 80% B in 8 min; Gradient 5: 5% B to 45% B in 8 min; Gradient 6: 5% B to 58% B in 8 min.

Condition 3: Column: Xselect CSH OBD Column 30×150 mm Sum; Mobile Phase A: Water (0.05% HCl) and Mobile Phase 2: ACN; Gradient 1: hold 3% B for 2 min, up to 20% B in 6 min; Gradient 2: hold 3% B for 2 min, up to 23% B in 8 min;

Condition 4: Column: C18 silica gel; Mobile phase A: Water (0.1% HCl), Mobile Phase B: ACN. Gradient 1: 10% B to 50% B in 10 min.

Condition 5: Column: C18 silica gel; Mobile phase A: Water (0.1% TFA), Mobile Phase B: ACN. Gradient 1: 5% B to 35% B in 12 min; Gradient 2: 20% B to 50% B in 15 min.

Condition 6: Column: Welch Ultimate XB-C18, 50*250 mm, 10 μm; Mobile Phase A: 0.1% TFA, Mobile Phase B: acetonitrile; Flow rate: 90 mL/min; Gradient 1: 5% B to 5% B in 2 min, Gradient 2: 5% B to 30% B in 12 min; Gradient 3: 10% B to 45% B in 12 min.

Condition 7: Column: Xselect C18, 19*150 mm, 5 μm; Mobile Phase A: 0.1% TFA, Mobile Phase B: acetonitrile; Flow rate: 20 mL/min; Gradient 1: 5% B to 30% B in 7 min.

Condition 8: Column: XBridge, C18 silica gel, 19*150 mm; Mobile phase A: water (0.05% NH₃H₂O), Mobile phase B: acetonitrile; Gradient 1: 20% B to 60% B in 12 min; Gradient 2: 25% B to 55% B in 7 min; Gradient 3: 25% B to 60% B in 8 min.

Condition 9: Column: YMC-Actus Triart C18, 30×150 mm, 5 μm; Mobile Phase A: Water (10 mmol/L NH₄HCO₃), Mobile Phase B: ACN; Flow rate: 60 mL/min; Gradient 1: 10% B to 28% B in 10 mi.

Condition 10: Column: C18 silica gel; Mobile phase A: Water (0.1% formic acid), Mobile Phase B: ACN. Gradient 1: 10% B to 100% B in 12 min.

Condition 11: Column: Xselect C18, 19*150 mm, 5 μm; Mobile Phase A: Water (10 mmol/L NH₄HCO₃), Mobile Phase B: acetonitrile; Flow rate: 20 mL/min; Gradient 1: 5% B to 65% B in 8 min; Gradient 2: 5% B in 36% B in 8 min.

Condition 12: Column: Phenomenex C18 75×30 mm×3 um; Mobile Phase A: Water (NH₄HCO₃), Mobile Phase B: Acetonitrile; Gradient 1: 40% B to 70% B in 8 min; Gradient 2: 25% B to 80% B in 8 min; Gradient 3: 30% B to 60% B in 8 min; Gradient 4: 15% B to 50% B in 8 min.

Condition 13: Column: Phenomenex C18 75×30 mm×3 um; Mobile Phase A: Water (HCl), Mobile Phase B: Acetonitrile; Gradient 1: 1% B to 25% B in 8 min; Gradient 2: 1% B to 45% B in 8 min; Gradient 3: 10% B to 45% B in 8 min.

Condition 14: Column: XBridge Prep OBD C18 150×40 mm×10 um; Mobile Phase A: water (NH₄HCO₃), Mobile Phase B: Acetonitrile; Gradient 1: 15% B to 45% B in 8 min; Gradient 2: 35% B to 65% B in 8 mi; Gradient 3: 30% B to 60% B in 8 min; Gradient 4: 25% B to 65% B in 8 min; Gradient 5: 60% B to 90% B in 8 min; Gradient 6: 75% to 95% B in 8 min.

Condition 15: Column: Xbridge BEH C18 100×30 mm×10 um; Mobile Phase A: Water(NH₄HCO₃), Mobile Phase B: Acetonitrile; Gradient 1: 30% B to 60% B in 8 min; Gradient 2: 25% B to 55% B in 10 min.

Preparative chiral HPLC: purification by chiral HPLC was performed on a Gilson-GX 281 using column: CHIRALPAK IG-3), CHIRALPAK IC-3 or CHIRALPAK OJ-3.

Condition 1: Column: CHIRAL ART Cellulose-SB, 4.6×100 mm, 3 μm; Mobile Phase A: Methyl tert-butyl ether (0.1% diethanolamine): EtOH=70

Condition 2: Column: CHIRALPAK IC, 2*25 cm, 5 μm; Mobile Phase A: hexane:DCM (1:1) (0.5% 2 M NH₃-MeOH), Mobile Phase B: EtOH; Flow rate: 24 mL/min; Gradient 1: 50% B to 50% B in 30 min.

Condition 3: Column: CHIRALPAK IG, 5*15 cm, 10 μm; Mobile Phase A: hexane: DCM (3: 1) (0.5% 2 M NH₃-MeOH), Mobile Phase B: EtOH; Flow rate: 80 mL/min; Gradient 1: 50% B to 50% B in 20 min.

Condition 4: Column: CHIRAL ART Cellulose-SB, 3×25 cm, 5 μm; Mobile Phase A: CO₂, Mobile Phase B: MeOH: ACN=1: 1(0.1% 2 M NH3-MeOH); Flow rate: 80 mL/min; Gradient 1: Isocratic 30% B; Gradient 2: Isocratic 15% B in 24 min.

Condition 5: Column: CHIRALPAK ID, 2×25 cm, 5 μm; Mobile Phase A: MTBE(0.1% DEA); Mobile Phase B: ethanol; Flow rate: 35 mL/min; Gradient 1: 15% B to 15% B in 14 min.

Condition 6: Column: CHIRALPAK IG, 5×15 cm, 10 μm; Mobile Phase A: Hexane (0.1% 2 M NH₃-MeOH); Mobile Phase B: Ethanol; Flow rate: 80 mL/min; Gradient a: 50% B to 50% B in 40 min.

Condition 7: Column: CHIRALPAK IF, 3×25 cm, 5 μm; Mobile Phase A: CO₂, Mobile Phase B: MeOH: DCM=2: 1(0.1% 2 M NH₃-MeOH); Flow rate: 80 mL/min; Gradient 1: isocratic 50% B;

Condition 8: Column: CHIRAL ART Cellulose-SJ, 3×25 cm, 5 μm; Mobile Phase A: CO₂, Mobile Phase B: MeOH(0.1% 2 M NH₃-MeOH); Flow rate: 80 mL/min; Gradient 1: isocratic 45% B.

Reverse flash chromatography: purification by reverse flash chromatography was performed according to the following conditions.

Condition 1: Column: C18 silica gel; Mobile Phase A: water (0.1% formic acid), Mobile Phase B: acetonitrile; Gradient 1: 40% B to 80% B in 12 min; Gradient 2: 30% B to 80% B in 12 min; Gradient 3: 30% B to 70% B in 12 min; Gradient 4: 5% B to 50% B in 12 min; Gradient 5: 5% B to 70% B.

Condition 2: Column: C18 silica gel; Mobile Phase A: water (0.1% NH₃H₂O), Mobile Phase B: acetonitrile; Gradient 1: 30% B to 80% B in 10 min; Gradient 2: 30% B to 50% B in 12 min; Gradient 3: 20% B to 50% B in 12 min; Gradient 4: 20% B to 70% B in 12 min; Gradient 5: 10% B to 55% B 12 min; Gradient 6: 20% B to 60% B in 10 min; Gradient 7: 50% B to 95% B in 12 min; Gradient 8: 20% B to 65% B in 10 min; Gradient 9: 10% B to 50% B in 10 min; Gradient 10: 15% B to 65% B in 10 min; Gradient 11: 5% B to 35% B in 12 min; Gradient 12: 10% B to 45% B in 10 min; Gradient 13: 10% B to 65% B in 10 min; Gradient 14: 20% B to 40% B in 10 min.

Condition 3: Column: C18 silica gel; Mobile Phase A: water, Mobile Phase B: acetonitrile; Gradient 1: 10% B to 50% B in 10 min; Gradient 2: 30% B to 70% B in 25 min

Condition 4: Column: Weich Ultimate XB-C18 50×25.0 mm, 10 μm; Mobile Phase A: water (0.1% NH₃·H₂O), Mobile Phase B: acetonitrile; Flow rate: 90 mL/min; Gradient 1: 15% B to 51% B in 12 min.

Condition 5: Column: C18 silica gel; Mobile Phase A: water (0.1% TFA), Mobile Phase B: acetonitrile; Gradient 1: 10% B to 50% B in 12 min; Gradient 2: 20% B to 40% B in 15 min; Gradient 3: 10% B to 35% B in 10 min; Gradient 4: 15% B to 51% B in 12 min; Gradient 5: 0% B to 50% B in 10 min; Gradient 6: 5% B to 50% B in 10 min.

Condition 6: Column: silica gel; Mobile Phase A: water (0.05% NH₃H₂O), Mobile Phase B: acetonitrile, Gradient 1: 10% B to 60% B in 10 min.

Condition 7: Column: Xselect CSH C18 OBD Column 30×150 mm 5 m, n; Mobile Phase A: Water (0.05% HCl), Mobile Phase B: ACN; Flow rate: 60 mL/min; Gradient 1: 3% B to 18% B in 8 min;

Condition 8: YMC-Actus Triart C18, 30×150 mm, 5 μm; Mobile Phase A: Water (10 mmol/L NH₄HCO₃), Mobile Phase B: ACN; Flow rate: 60 mL/min; Gradient 1: 20% B to 35% B in 12 min

Condition 9: Column: C18 silica gel; Mobile Phase A: water (0.1% HCl), Mobile Phase B: acetonitrile; Gradient 1: 10% B to 50% B in 10 min; Gradient 2: 5% B to 50% B in 10 min; Gradient 3: 5% B to 70% B in 10 min.

Condition 10: Column: C18 silica gel; Mobile Phase A: water; Mobile Phase B: methanol; Gradient 1: 10% B to 50% B in 10 min.

### General Synthetic Schemes

Compounds of the present disclosure may be prepared using a synthetic protocol illustrated below in Schemes A and B.

### Steps 1 and 2

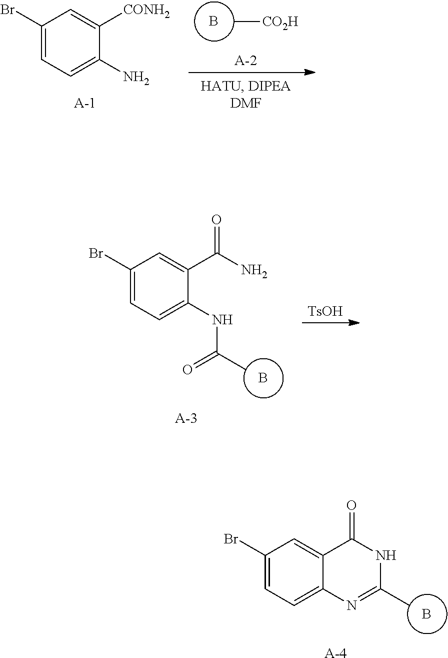

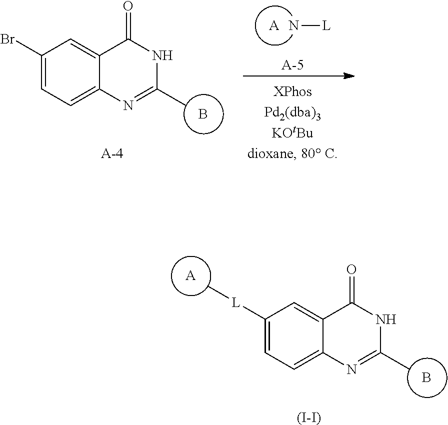

Scheme A. An exemplary method of preparing a representative compound of Formula (I); wherein A, B, and L are as defined herein.

An exemplary method of preparing a compound of Formula (I-I) is provided in Scheme A. In this scheme, A-3 is prepared in Step 1 by incubating A-1 with A-2 in the presence of hexafluorophosphate azabenzotriazole tetramethyl uranium (HATU), or a similar coupling agent, diisopropylethylamine (DIPEA), and dimethylformamide (DMF). Suitable alternatives to DIPEA and DMF may also be used in the reaction. In Step 2, A-3 is cyclized by treatment with tosic acid, or a similar alternative, in order to provide A4.

Next, in Step 3, A-4 is coupled with A-5 to provide a compound of Formula (I-I). This coupling reaction may be conducted in the presence of Pd₂(dba)₃, XPhos, and KOtBu or a similar reagent. Alternative catalysts to Pd₂(dba)₃ may also be used, such as any suitable palladium catalyst. Likewise, other ligands similar to XPhos may be implemented in the reaction of Step 3. The reaction of Step 3 is carried out in dioxane, or a similar solvent, and the reaction is heated to 80° C. or a temperature sufficient to provide the compound of Formula (I-I). Each starting material and/or intermediate in Scheme A may be protected and deprotected using standard protecting group methods. In addition, purification and characterization of each intermediate as well as the final compound of Formula (I) may be afforded by any accepted procedure.

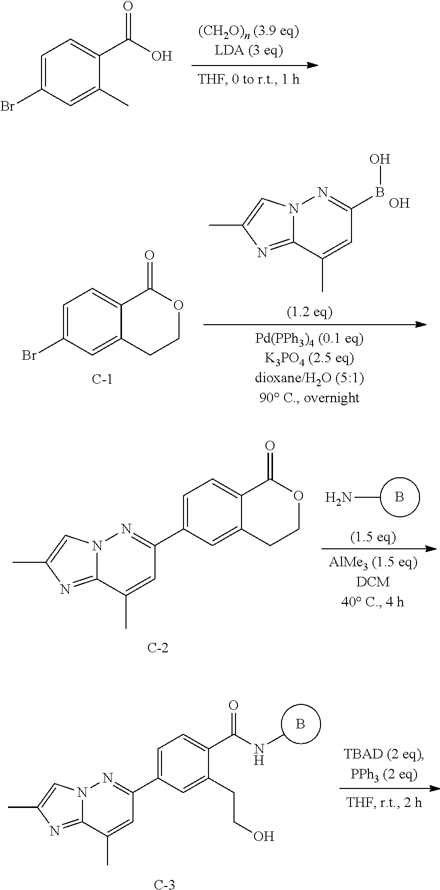

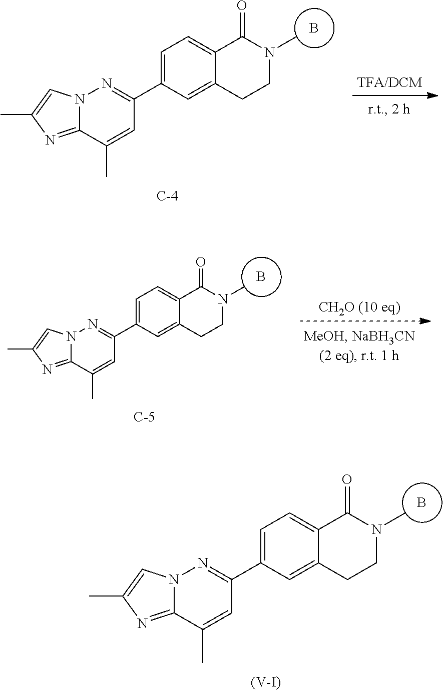

Scheme C. An exemplary method of preparing a representative compound of Formula (V-I); wherein B is as defined herein.

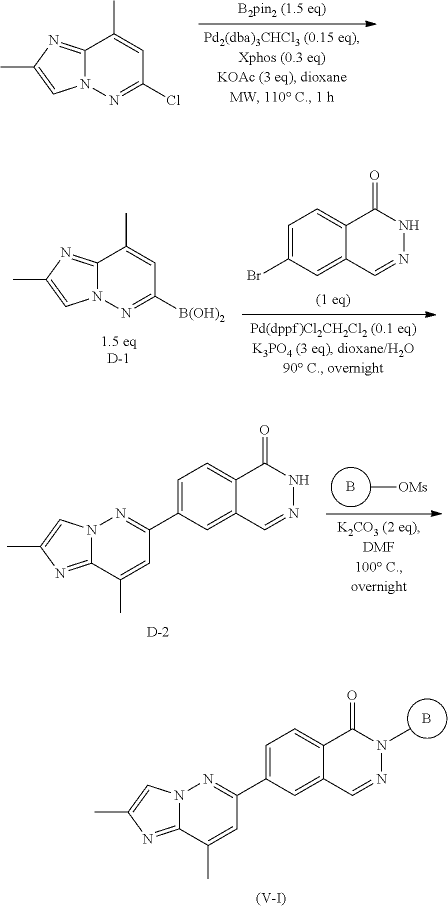

Scheme D. An exemplary method of preparing a representative compound of Formula (V-I); wherein B is as defined herein.

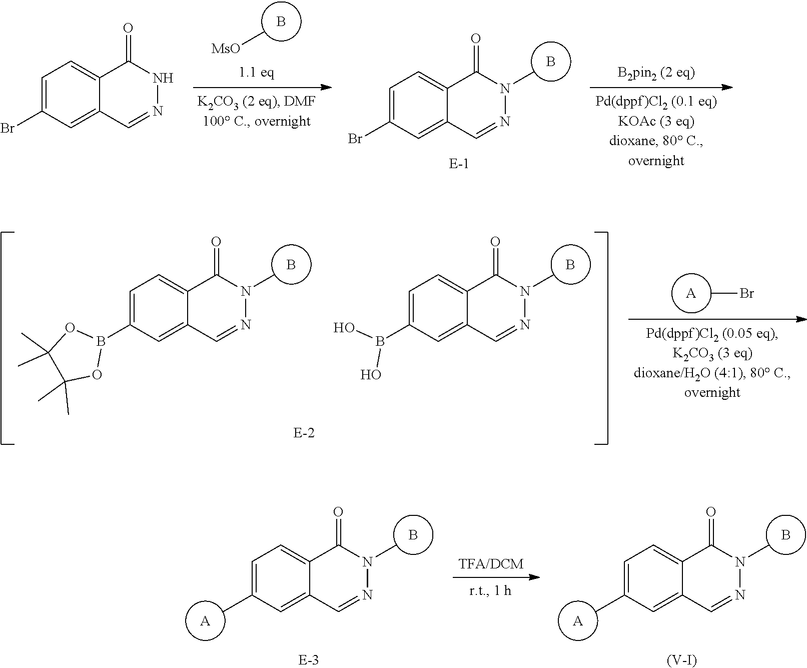

Scheme E. An exemplary method of preparing a representative compound of Formula (V-I); wherein each of A and B is as defined herein. The last step of this scheme involves deprotection of an acid-labile nitrogen protecting group, if needed (e.g., Boc).

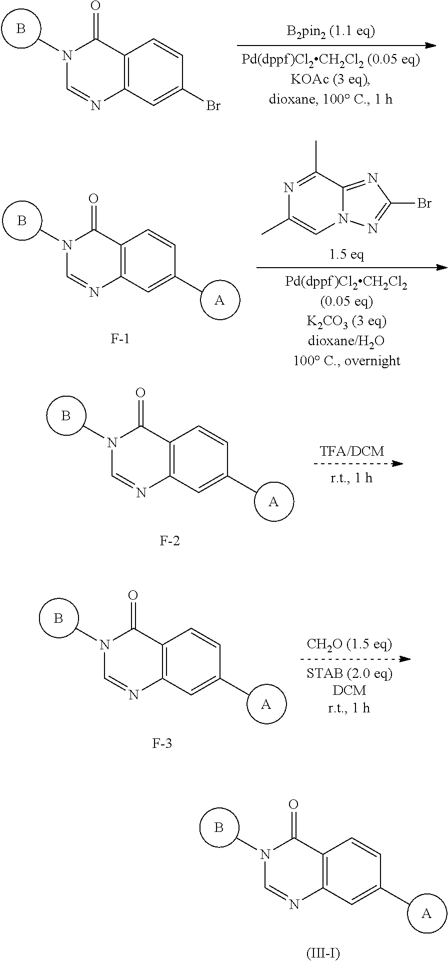

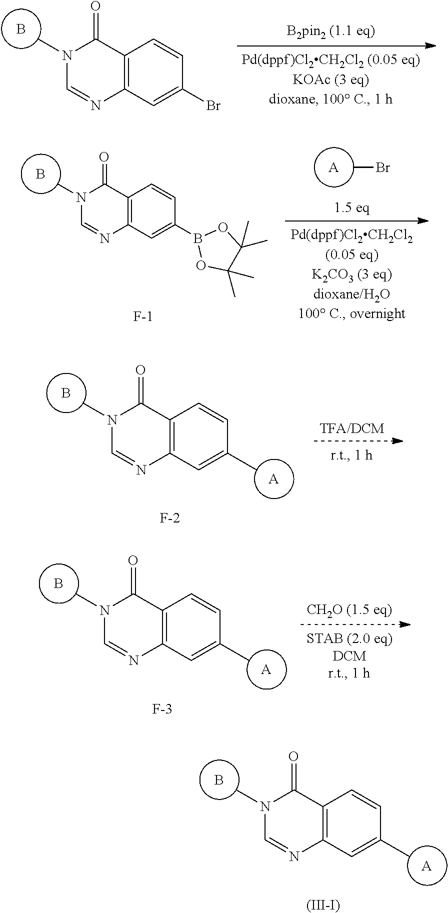

Scheme F. An exemplary method of preparing a representative compound of Formula (III-I); wherein each of A and B is as defined herein. The last steps of this scheme involve deprotection of an acid-labile nitrogen protecting group, if needed (e.g., Boc), and optional methylation.

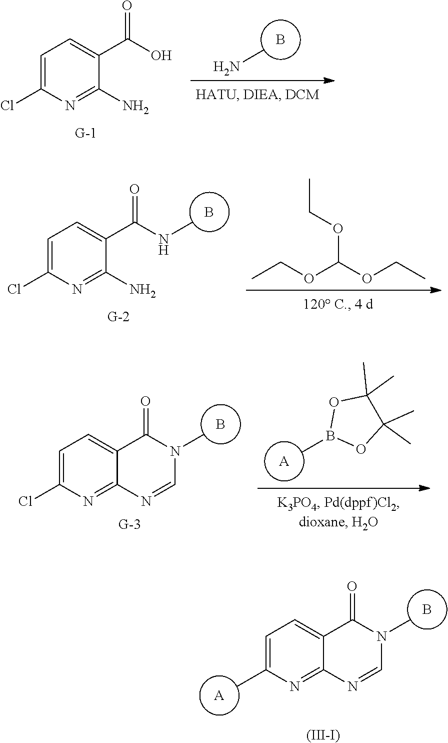

Scheme G. An exemplary method of preparing a representative compound of Formula (III-I); wherein each of A and B is as defined herein. The last steps of this scheme involve deprotection of an acid-labile nitrogen protecting group, if needed (e.g., Boc), and optional methylation.

Schemes and protocols for the synthesis of exemplary compounds disclosed herein from Tables 1-4, which represent Examples 1-89, can be found in WO 2021/207554. Schemes and protocols for the synthesis of exemplary compounds disclosed herein from Tables 8-9 can be found in WO 2021/207532. Each of the foregoing references are incorporated herein by reference in its entirety.

### Example 90: Synthesis of Compound 346

### Synthesis of Intermediate C1

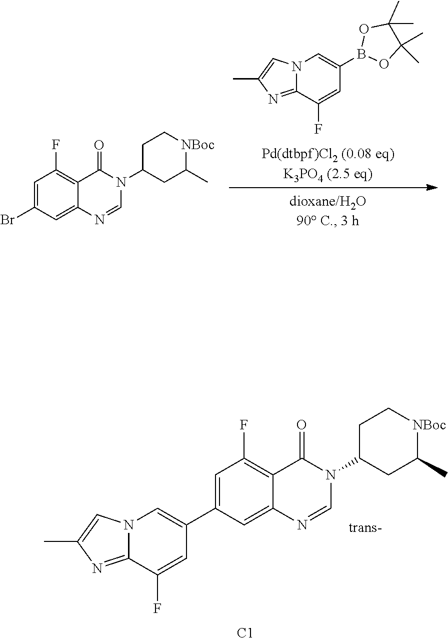

A mixture of tert-butyl 4-(7-bromo-5-fluoro-4-oxoquinazolin-3-yl)-2-methylpiperidine-1-carboxylate (140 mg, 0.318 mmol, 1.00 equiv), 8-fluoro-2-methyl-6-(4,4,5,5-tetramethyl-1,3,2-dioxaborolan-2-yl)imidazo[1,2-a]pyridine (131.69 mg, 0.477 mmol, 1.5 equiv), Pd(DtBPF)Cl₂ (20.72 mg, 0.032 mmol, 0.1 equiv), and K₃PO₄ (202.47 mg, 0.954 mmol, 3 equiv) in 1,4-dioxane (4 mL) and water (1 mL) was stirred for 3 hr at 90° C. under nitrogen atmosphere. The resulting mixture was concentrated under vacuum to give a residue. The residue was applied onto a silica gel column with ethyl acetate/hexane (1:1), followed by chiral HPLC to afford trans-tert-butyl 4-(5-fluoro-7-{8-fluoro-2-methylimidazo[1,2-a]pyridin-6-yl}-4-oxoquinazolin-3-yl)-2-methylpiperidine-1-carboxylate (80 mg, 49%) (C1; 20 mg) as a solid. LCMS (ES, m/z): 510 [M+H]⁺.

### Synthesis of Compound 346

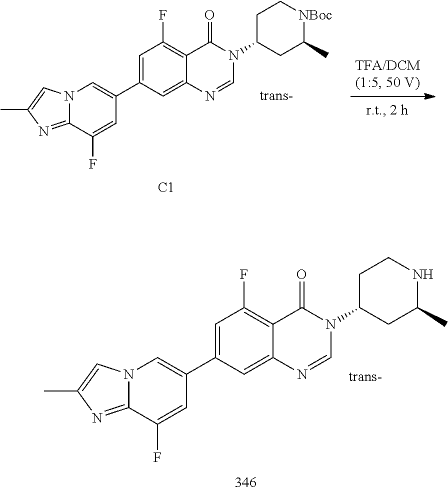

A mixture of trans-tert-butyl-4-(5-fluoro-7-{8-fluoro-2-methylimidazo[1,2-a]pyridin-6-yl}-4-oxoquinazolin-3-yl)-2-methylpiperidine-1-carboxylate (20 mg, 0.039 mmol, 1.00 equiv), dichloromethane (1.5 mL), and trifluoroacetic acid (0.25 mL) was stirred for 2 hr at room temperature, then concentrated under vacuum to give a residue. The residue was purified by prep-HPLC (Condition 2, Gradient 1) to afford trans-5-fluoro-7-(8-fluoro-2-methylimidazo[1,2-a]pyridin-6-yl)-3-(2-methylpiperidin-4-yl)quina-zolin-4(3H)-one (Compound 346, 6 mg, 38%) as a solid. LCMS (ES, m/z): 410 [M+H]^{+ 1}H NMR (400 MHz, DMSO-d₆) δ 9.06 (d, J=1.6 Hz, 1H), 8.48 (s, 1H), 7.88 (d, J=1.8 Hz, 1H), 7.84 (d, J=3.1 Hz, 1H), 7.74 (ddd, J=12.6, 4.2, 1.7 Hz, 2H), 4.98 (tt, J=11.8, 4.0 Hz, 1H), 3.34 (s, 1), 2.96-2.81 (m, 2H), 2.39 (s, 3H), 2.05 (td, J 12.1, 5.0 Hz, 1H), 1.89 (qd, J=11.5, 4.4 Hz, 1H), 1.82-1.74 (m, 1H), 1.66 (ddt, J=12.3, 4.3, 2.2 Hz, 1H), 1.20 (d, J=6.9 Hz, 3). ¹⁹F NMR (376 MHz, DMSO) δ 110.22, 131.85.

### Example 91: Synthesis of Compounds 338, 339, and 341

### Synthesis of Intermediate C2

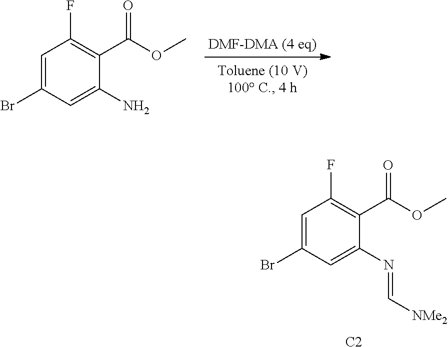 [SMILES: COC(=O)c1c(N)cc(Br)cc1F ; "DMF-DMA (4 eq)" ; COC(=O)c1c(F)cc(Br)cc1/N=C/N(C)C ; "C2"]

A mixture of methyl 2-amino-4-bromo-6-fluorobenzoate (500 mg, 2.02 mmol, 1.00 equiv), N,N-dimethyl formamide dimethyl acetal (963.5 mg, 8.08 mmol, 4.0 equiv), and toluene (5 mL) was stirred for 4 hr at 100° C. The resulting mixture was concentrated under vacuum to afford methyl (E)-4-bromo-2-(((dimethylamino)methylene)amino)-6-fluorobenzoate (510 mg) as an oil. LCMS (ES, m/z): 303 [M+H]⁺.

### Synthesis of Intermediate C₃

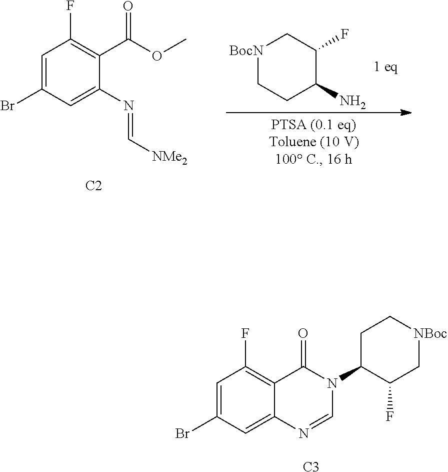

A mixture of methyl (E)-4-bromo-2-(((dimethylamino)methylene)amino)-6-fluorobenzoate (150.0 mg, 0.49 mmol, 1.00 equiv), trans-tert-butyl-4-amino-3-fluoropiperidine-1-carboxylate (107.9 mg, 0.49 mmol, 1.0 equiv), p-toluenesulfonic acid (8.46 mg, 0.049 mmol, 0.1 equiv), and toluene (1.5 mL) was stirred for 16 hr at 100° C. The resulting mixture was concentrated under vacuum to give a residue, then quenched with water (20 mL) and extracted with ethyl acetate (3×20 mL). The organic layers were combined, washed with saturated aqueous NaCl (1×50 mL), dried over anhydrous sodium sulfate, and filtered. The filtrate was concentrated under vacuum to give a residue. The residue was applied onto a silica gel column with ethyl acetate/petroleum ether to afford trans-tert-butyl-4-(7-bromo-5-fluoro-4-oxoquinazolin-3(4H)-yl)-3-fluoropiperidine-1-carboxylate (100 mg) as a solid. LCMS (ES, m/z): 444 [M+H]⁺.

### Synthesis of Intermediate C₄

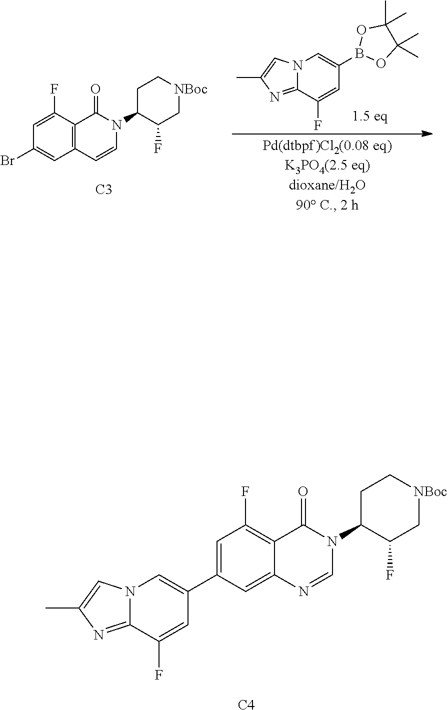

A mixture of trans-tert-butyl-4-(7-bromo-5-fluoro-4-oxoquinazolin-3(4H)-yl)-3-fluoropiperidine-1-carboxylate (100.0 mg, crude), 8-fluoro-2-methyl-6-(4,4,5,5-tetramethyl-1,3,2-dioxaborolan-2-yl)imidazo[1,2-a]pyridine (93.4 mg, 0.338 mmol, 1.50 equiv), dioxane (3 mL), K₃PO₄ (119.6 mg, 0.56 mmol, 2.50 equiv), H₂O (0.6 mL), and Pd(dtbpf)Cl₂ (11.7 mg, 0.018 mmol, 0.08 equiv) was stirred for 2 hr at 90° C. under nitrogen atmosphere. The reaction mixture was quenched with water (15 mL) and extracted with ethyl acetate (3×20 mL). The organic layers were combined, washed with saturated aqueous NaCl (1×50 mL), dried over anhydrous sodium sulfate, and filtered. The filtrate was concentrated under vacuum to give a residue. The residue was applied onto a silica gel column with ethyl acetate/petroleum ether to afford of trans tert-butyl-3-fluoro-4-(5-fluoro-7-(8-fluoro-2-methylimidazo[1,2-a]pyridin-6-yl)-4-oxoquinazolin-3(4H)-yl)piperidine-1-carboxylate (C4, 20 mg, 17%) as a solid. LCMS (ES, m/z): 514 [M+H]⁺.

### Synthesis of Compound 341

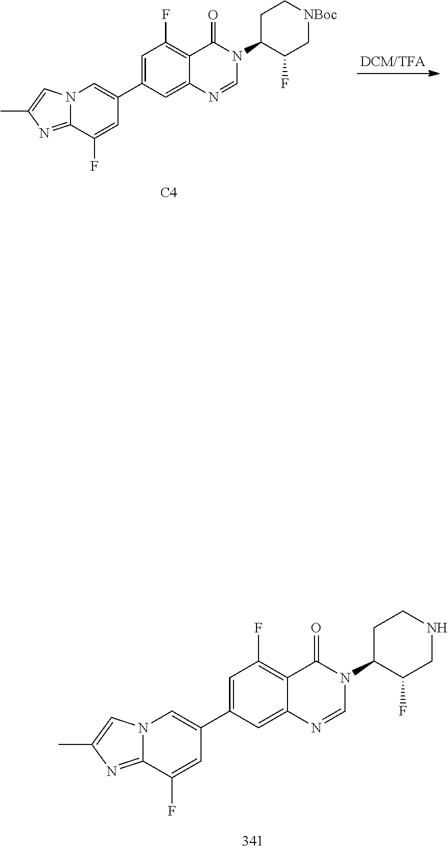

A mixture of trans tert-butyl-3-fluoro-4-(5-fluoro-7-(8-fluoro-2-methylimidazo[1,2-a]pyridin-6-yl)-4-oxoquinazolin-3(4H)-yl)piperidine-1-carboxylate (20.0 mg, 0.038 mmol, 1.00 equiv), dichloromethane (1.0 mL), and trifluoroacetic acid (0.2 mL) was stirred for 30 min at room temperature. The resulting mixture was concentrated under vacuum to give a residue. The residue was purified by Prep-HPLC (Condition 1, Gradient 2) to afford 5-fluoro-7-(8-fluoro-2-methylimidazo[1,2-a]pyridin-6-yl)-3-(trans-3-fluoropiperidin-4-yl)quinazolin-4(3H)-one (Compound 341, 5.1 mg, 31%) as a solid.

Compounds 338 and 339 were synthesized according to the method of Compound 341 using the intermediates show in the Table below.

| Compound No. | Intermediate | Analysis data |
|---|---|---|
| 341 | 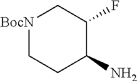 [SMILES: CC(C)(C)OC(=O)N1CC[C@H](N)[C@@H](F)C1] | LCMS: (ES, m/z): 414 [M + H]⁺ ¹H NMR: (400 MHZ, DMSO-d₆) δ 9.09 (d, J = 1.6 Hz, 1H), 8.60 (s, 1H), 7.93 (d, J = 1.8 Hz, 1H), 7.87-7.83 (m, 1H), 7.78 (td, J = 12.5, 1.7 Hz, 2H), 5.21-4.91 (m, 1H), 4.78 (s, 1H), 3.39 (m, 1H), 2.99 (d, J = 12.4 Hz, 1H), 2.56 (t, J = 8.4 Hz, 2H), 2.42-2.37 (m, 3H), 2.08 (d, J = 12.7 Hz, 1H), 1.92 (d, J = 11.6 Hz, 1H). |
| 338 | 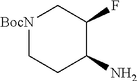 [SMILES: CC(C)(C)OC(=O)N1CC[C@H](N)[C@H](F)C1] | LCMS: (ES, m/z): 414 [M-HCl]⁺ ¹H NMR: (400 MHZ, DMSO-d₆) δ 9.63 (d, J = 10.9 Hz, 1H), 9.36 (s, 1H), 8.96 (s, 1H), 8.34 (s, 1H), 8.28 (d, J = 11.8 Hz, 1H), 8.02 (d, J = 1.8 Hz, 2H), 7.88 (dd, J = 12.2, 1.8 Hz, 1H), 5.37-5.26 (m, 2H), 3.67 (d, J = 12.4 Hz, 2H), 3.46 (d, J = 13.0 Hz, 1H), 3.44 (d, 1H), 3.34 (t, J = 12.0 Hz, 1H), 2.60 (m, 3H), 2.09 (d, J = 13.1 Hz, 1H). |
| 339 | 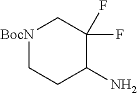 [SMILES: CC(C)(C)OC(=O)N1CCC(N)C(F)(F)C1] | LCMS: (ES, m/z): 432 [M-HCl]⁺ ¹H NMR: (400 MHZ, DMSO-d₆) δ 10.19 (s, 1H), 9.55 (s, 1H), 9.31 (d, J = 8.2 Hz, 1H), 8.38 (d, J = 2.3 Hz, 1H), 8.16 (d, J = 12.4 Hz, 1H), 8.02 (d, J = 1.8 Hz, 2H), 7.89 (dd, J = 12.3, 1.7 Hz, 1H), 5.79 (dd, J = 26.8, 12.8 Hz, 1H), 3.99 (t, J = 12.1 Hz, 2H), 3.43 (t, J = 12.1 Hz, 2H), 2.64 (s, 1H), 2.51-2.45 (m, 3H), 2.38 (s, 1H). |

### Example 92: Synthesis of Compounds 342 and 344

### Synthesis of Intermediate C5

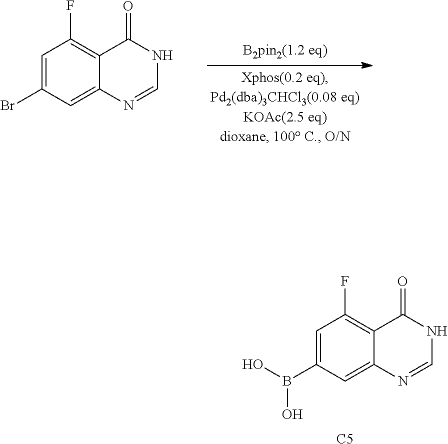

A mixture of 7-bromo-5-fluoro-3H-quinazolin-4-one (5.0 g, 20.57 mmol, 1.00 equiv), B₂pin₂ (6.3 g, 24.68 mmol, 1.2 equiv), XPhos (1.96 g, 4.11 mmol, 0.2 equiv), Pd₂(dba)₃CHCl₃ (1.7 g, 1.64 mmol, 0.08 equiv), and potassium acetate (5.1 g, 51.43 mmol, 2.5 equiv) in dioxane (50 mL) was stirred for overnight at 100° C. under a nitrogen atmosphere. LCMS (ES, m/z): 209 [M+H]⁺.

### Synthesis of Intermediate C6

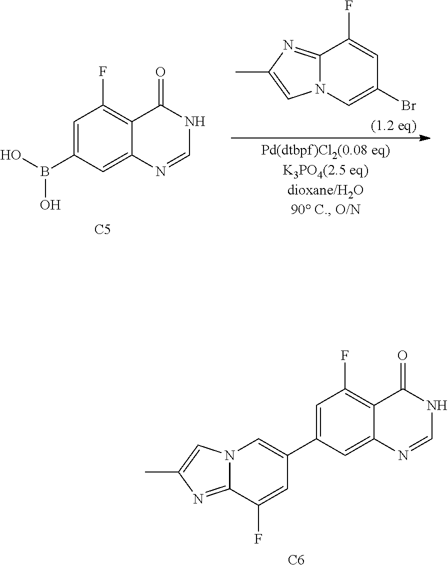

A mixture of 5-fluoro-4-oxo-3H-quinazolin-7-ylboronic acid (crude),6-bromo-8-fluoro-2-methylimidazo[1,2-a]pyridine (6.61 g, 28.85 mmol, 1.2 equiv), Pd(DtBPF)Cl₂ (1.25 g, 1.92 mmol, 0.08 equiv), and K₃PO₄ (12.76 g, 60.10 mmol, 2.5 equiv) in dioxane (50 mL) and H₂O (12 mL) was stirred for overnight at 90° C. under a nitrogen atmosphere. The resulting mixture was diluted with water (100 mL). A precipitate formed that was collected by filtration and washed with MeOH/H₂O(1:1) (5×50 mL) to afford 5-fluoro-7-{8-fluoro-2-methylimidazo[1,2-a]pyridin-6-yl}-3H-quinazolin-4-one (C6, 4.5 g, 59%) as a solid. LCMS (ES, m/z): 313 [M+H]⁺.

### Synthesis of Intermediate C7

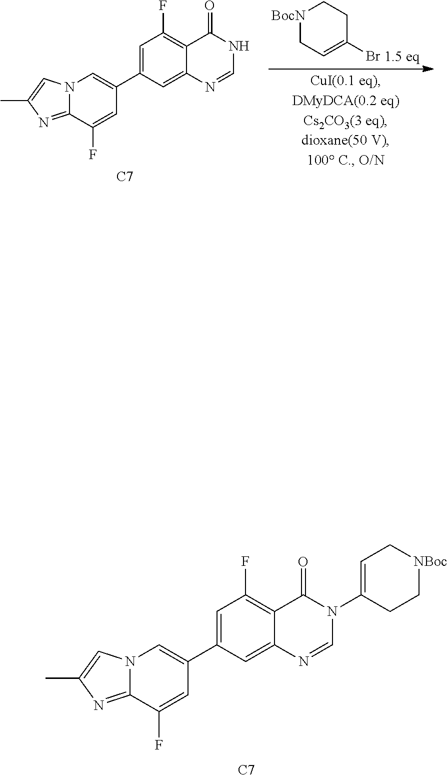

A mixture of 5-fluoro-7-{8-fluoro-2-methylimidazo[1,2-a]pyridin-6-yl}-3H-quinazolin-4-one (1.0 g, 3.20 mmol, 1.00 equiv), tert-butyl 4-bromo-3,6-dihydro-2H-pyridine-1-carboxylate (1.3 g, 4.80 mmol, 1.5 equiv), CuI (0.06 g, 0.32 mmol, 0.1 equiv), DMyDCA (0.09 g, 0.64 mmol, 0.2 equiv), and Cs₂CO₃ (3.1 g, 9.60 mmol, 3 equiv) in dioxane (50 mL) was stirred for overnight at 100° C. The resulting mixture was diluted with water (100 mL) and extracted with ethyl acetate (3×100 mL). The organic layers were combined, washed with brine (1×100 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (96:4) to afford tert-butyl 4-(5-fluoro-7-{8-fluoro-2-methylimidazo[1,2-a]pyridin-6-yl}-4-oxoquinazolin-3-yl)-3,6-dihydro-2H-pyridine-1-carboxylate (C7, 1 g, 63%) as an oil. LCMS (ES, m/z): 494 [M+H]⁺.

### Synthesis of Compound 342

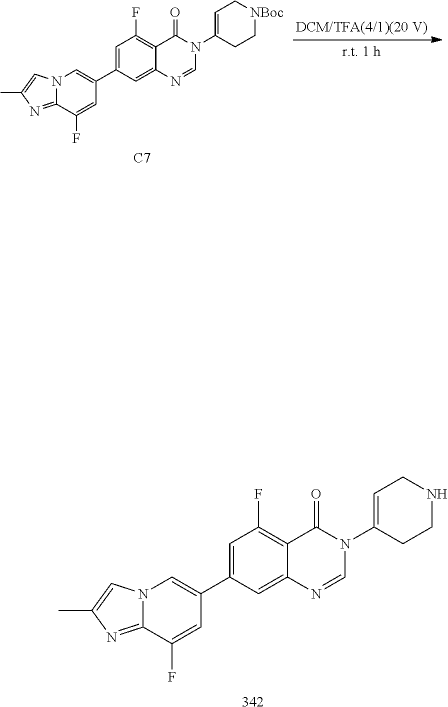 [SMILES: Cc1cn2cc(-c3cc(F)c4c(=O)n(C5=CCN(C(=O)OC(C)(C)C)CC5)cnc4c3)cc(F)c2n1 ; "C7" ; Cc1cn2cc(-c3cc(F)c4c(=O)n(C5=CCNCC5)cnc4c3)cc(F)c2n1 ; "342"]

A mixture of tert-butyl 4-(5-fluoro-7-{8-fluoro-2-methylimidazo[1,2-a]pyridin-6-yl}-4-oxoquinazolin-3-yl)-3,6-dihydro-2H-pyridine-1-carboxylate (150 mg, 0.30 mmol, 1.00 equiv) in trifluoroacetic acid (1 mL) and dichloromethane (4 mL) was stirred for 1 hr at room temperature. The resulting mixture was concentrated under vacuum to give a residue. The residue was purified by Prep-HPLC (Condition 2, Gradient 2) to afford 5-fluoro-7-{8-fluoro-2-methylimidazo[1,2-a]pyridin-6-yl}-3-(1,2,3,6-tetrahydropyridin-4-yl)quinazolin-4-one (Compound 344, 5.4 mg, 4%) as solid. LCMS: (ES, m/z): 394 [M+H]^{+ 1}H NMR: (400 MHz, DMSO-d₆) δ 8.90 (s, 1H), 8.14 (s, 1H), 7.84-7.79 (m, 2H), 7.61 (dd, J=35.5, 12.3 Hz, 2H), 5.98 (s, 1H), 3.40 (q, J=3.0 Hz, 2H), 2.95 (t, J=5.6 Hz, 2H), 2.38 (s, 3H), 2.32 (s, 2H). ¹⁹F NMR (376 MHz, DMSO) δ−73.41, −109.46, −109.72, −110.15, −131.59, −131.83, −131.88, −220.61.

### Synthesis of Intermediate C8

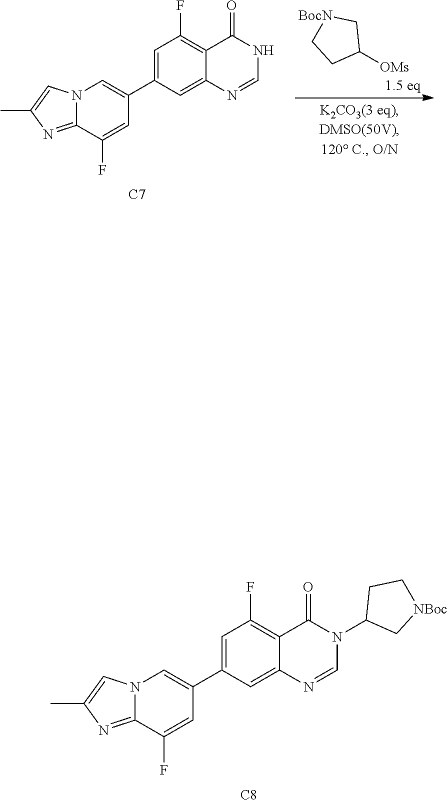

A mixture of 5-fluoro-7-{8-fluoro-2-methylimidazo[1,2-a]pyridin-6-yl}-3H-quinazolin-4-one (150 mg, 0.48 mmol, 1.00 equiv), tert-butyl 3-(methanesulfonyloxy)pyrrolidine-1-carboxylate (191.1 mg, 0.72 mmol, 1.5 equiv), and K₂CO₃ (199.1 mg, 1.44 mmol, 3 equiv) in DMSO (5 mL) was stirred for overnight at 110° C. The resulting mixture was diluted with water (30 mL) and extracted with ethyl acetate (3×30 mL). The organic layers were combined, washed with brine (1×30 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (96:4) to afford tert-butyl 3-(5-fluoro-7-{8-fluoro-2-methylimidazo[1,2-a]pyridin-6-yl}-4-oxoquinazolin-3-yl)pyrrolidine-1-carboxylate (C8, 24 mg, 10%) as a solid. LCMS (ES, m/z): 482 [M+H]⁺.

### Synthesis of Compound 344

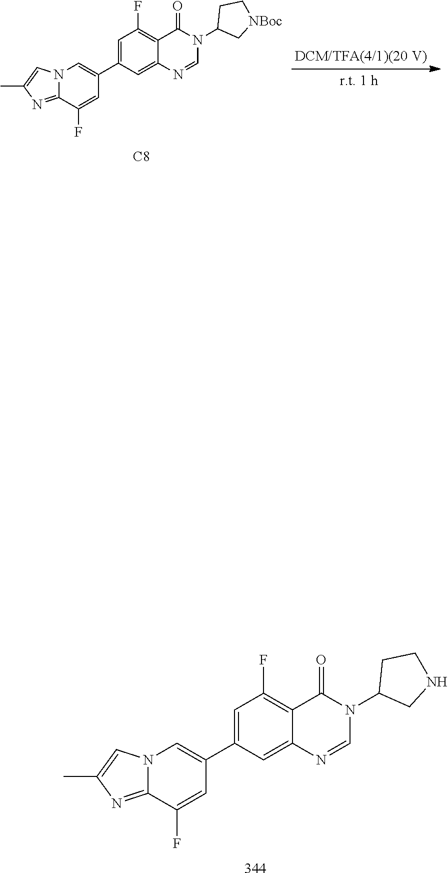 [SMILES: Cc1cn2cc(-c3cc(F)c4c(=O)n(C5CCN(C(=O)OC(C)(C)C)C5)cnc4c3)cc(F)c2n1 ; "C8" ; Cc1cn2cc(-c3cc(F)c4c(=O)n(C5CCNC5)cnc4c3)cc(F)c2n1 ; "344"]

A mixture of tert-butyl 3-(5-fluoro-7-{8-fluoro-2-methylimidazo[1,2-a]pyridin-6-yl}-4-oxoquinazolin-3-yl)pyrrolidine-1-carboxylate (24.0 mg, 0.05 mmol, 1.00 equiv) in TFA (0.2 mL) and DCM (0.8 mL) was stirred for 1 hr at room temperature. The resulting mixture was concentrated under vacuum to give a residue. The residue was purified by Prep-HPLC (Condition 2, Gradient 2) to afford 5-fluoro-7-{8-fluoro-2-methylimidazo[1,2-a]pyridin-6-yl}-3-(pyrrolidin-3-yl)quinazolin-4-one (Compound 344, 4.4 mg, 21%) as a solid. LCMS: (ES, m/z): 382 [M+H]^{+ 1}H NMR:(400 MHz, DMSO-d₆) δ 8.86 (d, J=1.6 Hz, 1H), 8.39 (s, 1H), 7.80 (dd, J=12.3, 2.3 Hz, 2H), 7.62 (dd, J=12.4, 1.8 Hz, 1H), 7.54 (dd, J=12.4, 1.6 Hz, 1H), 5.15-5.04 (m, 1H), 3.20-3.06 (m, 2H), 2.97 (dd, J=11.8, 4.3 Hz, 1H), 2.86 (ddd, J=10.7, 8.2, 6.7 Hz, 1H), 2.37 (d, J=0.9 Hz, 3H), 2.25 (dtd, J=13.7, 8.6, 5.3 Hz, 1H), 1.97-1.84 (m, 1H). 1⁹F NMR (376 MHz, DMSO) δ−73.40, −110.15, −110.21, −131.46, −131.86, −131.88, −131.93, −132.20.

### Example 93: Synthesis of Compounds 347 and 348

### Synthesis of Intermediate C9 and C10

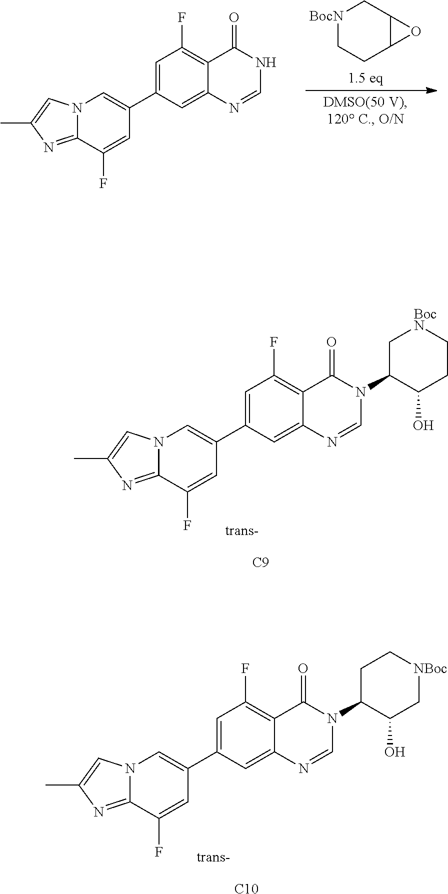 [SMILES: Cc1cn2cc(-c3cc(F)c4c(=O)[nH]cnc4c3)cc(F)c2n1 ; CC(C)(C)OC(=O)N1CCC2OC2C1 ; Cc1cn2cc(-c3cc(F)c4c(=O)n([C@H]5CN(C(=O)OC(C)(C)C)CC[C@@H]5O)cnc4c3)cc(F)c2n1 ; "C9" ; Cc1cn2cc(-c3cc(F)c4c(=O)n([C@H]5CCN(C(=O)OC(C)(C)C)C[C@@H]5O)cnc4c3)cc(F)c2n1 ; "C10"]

A mixture of 5-fluoro-7-{8-fluoro-2-methylimidazo[1,2-a]pyridin-6-yl}-3H-quinazolin-4-one (1.15 g, 3.68 mmol, 1.00 equiv) and K₂CO₃ (1.53 g, 11.04 mmol, 3 equiv) in DMSO (38.33 mL) was stirred for overnight at 110° C. The resulting mixture was diluted with water (30 mL) and extracted with ethyl acetate (3×30 mL). The organic layers were combined, washed with brine (1×30 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (96:4) to afford mixture products as a solid. LCMS (ES, m/z): 512 [M+H]⁺.

### Synthesis of Compounds 347 and 348

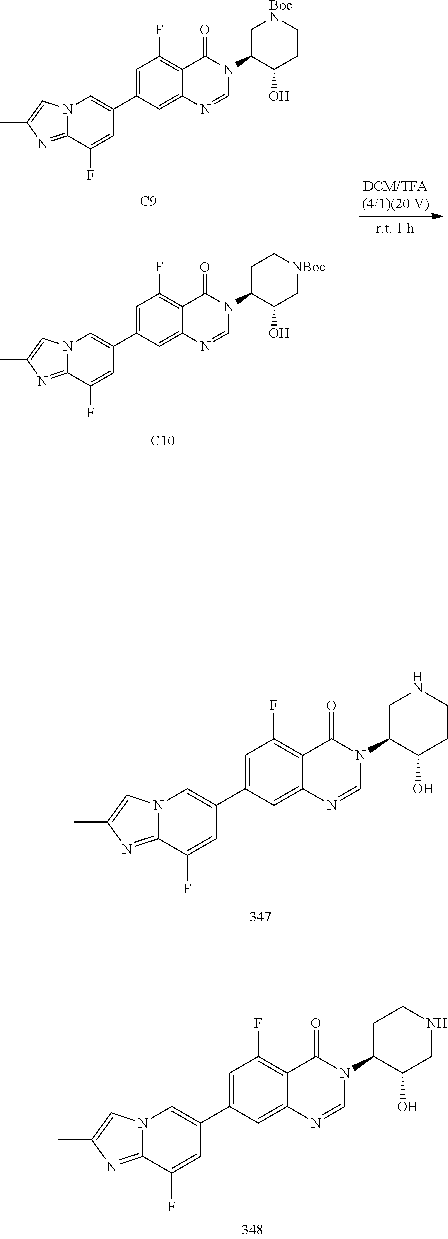 [SMILES: Cc1cn2cc(-c3cc(F)c4c(=O)n([C@H]5CN(C(=O)OC(C)(C)C)CC[C@@H]5O)cnc4c3)cc(F)c2n1 ; "C9" ; Cc1cn2cc(-c3cc(F)c4c(=O)n([C@H]5CCN(C(=O)OC(C)(C)C)C[C@@H]5O)cnc4c3)cc(F)c2n1 ; "C10" ; Cc1cn2cc(-c3cc(F)c4c(=O)n([C@H]5CNCC[C@@H]5O)cnc4c3)cc(F)c2n1 ; Cc1cn2cc(-c3cc(F)c4c(=O)n([C@H]5CCNC[C@@H]5O)cnc4c3)cc(F)c2n1]

A mixture of C9 and C10 (370.0 mg, 0.72 mmol, 1.00 equiv) in trifluoroacetic acid (1 mL) and dichloromethane (4 mL) was stirred for 1 hr at room temperature. The resulting mixture was concentrated under vacuum to give a residue. The residue was purified by Prep-HPLC (Condition 1, Gradient 3) to afford 5-fluoro-7-{8-fluoro-2-methylimidazo[1,2-a]pyridin-6-yl}-3-[(3S,4S)-3-hydroxypiperidin-4-yl]quinazolin-4-one (Compound 347, 8.6 mg, 2%) as a solid and product 5-fluoro-7-{8-fluoro-2-methylimidazo[1,2-a]pyridin-6-yl}-3-[(3S,4S)-4-hydroxypiperidin-3-yl]quinazolin-4-one (Compound 348, 8.7 mg, 2%) as a solid.

| Compound | |
|---|---|
| No. | Analysis Data |
| 347 | ¹H NMR (400 MHz, DMSO-d₆) δ 9.08 (d, J = 1.6 Hz, 1H), 8.43 (s, |
| | 1H), 7.89 (d, J = 1.8 Hz, 1H), 7.84 (d, J = 3.0 Hz, 1H), 7.75 (dd, J = |
| | 12.5, 1.6 Hz, 2H), 5.12 (d, J = 5.2 Hz, 1H), 4.42 (s, 1H), 3.95 (s, 1H), |
| | 3.16 (dd, J = 11.9, 4.7 Hz, 1H), 2.99 (d, J = 12.2 Hz, 1H), 2.55 (d, J = |
| | 11.2 Hz, 1H), 2.39 (s, 3H), 2.37-2.32 (m, 1H), 1.96 (s, 1H), 1.81 (dd, |
| | J = 12.7, 3.9 Hz, 1H). |
| | ¹⁹F NMR (376 MHz, DMSO) δ −73.40, −110.18, −131.86. |
| 348 | LCMS (ES, m/z)(PH-RMT-2021-0222-0A): 412 [M + H]⁺ |
| | ¹H NMR (400 MHz, DMSO-d₆) δ 9.06 (d, J = 1.6 Hz, 1H), 8.47 (s, |
| | 1H), 7.88 (d, J = 1.8 Hz, 1H), 7.82 (d, J = 3.0 Hz, 1H), 7.74 (dd, J = |
| | 12.5, 1.7 Hz, 2H), 5.31-4.99 (m, 1H), 4.44 (s, 1H), 4.06 (s, 1H), 3.08- |
| | 2.87 (m, 2H), 2.84 (s, 1H), 2.57 (td, J = 12.9, 2.6 Hz, 1H), 2.39 (s, |
| | 3H), 2.01-1.83 (m, 1H), 1.45 (qd, J = 12.4, 4.0 Hz, 1H). |
| | ¹⁹F NMR (376 MHz, DMSO) δ −73.43, −110.61, −132.11. |

### Example 94: Synthesis of Compound 352

### Synthesis of Intermediate C13

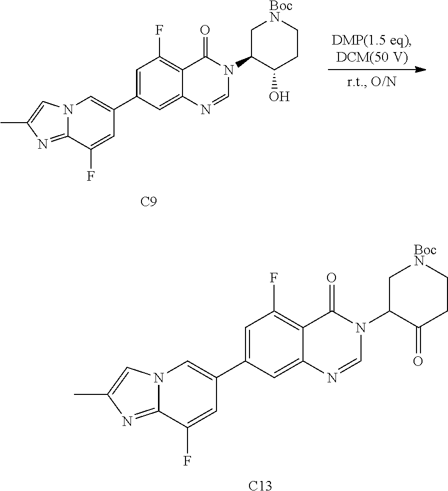 [SMILES: Cc1cn2cc(-c3cc(F)c4c(=O)n([C@H]5CN(C(=O)OC(C)(C)C)CC[C@@H]5O)cnc4c3)cc(F)c2n1 ; "C9" ; Cc1cn2cc(-c3cc(F)c4c(=O)n(C5CN(C(=O)OC(C)(C)C)CCC5=O)cnc4c3)cc(F)c2n1 ; "C13"]

A mixture of tert-butyl (3S,4S)-3-(5-fluoro-7-{8-fluoro-2-methylimidazo[1,2-a]pyridin-6-yl}-4-oxoquinazolin-3-yl)-4-hydroxypiperidine-1-carboxylate (40.0 mg, 0.08 mmol, 1.00 equiv) and DMP (49.7 mg, 0.12 mmol, 1.5 equiv) in dichloromethane (2 mL) was stirred overnight at room temperature. The resulting mixture was diluted with Na₂S₂O₃ (2 mL), basified to pH 8 with saturated NaHCO₃ (aq.), and extracted with CH₂Cl₂ (3×5 mL). The organic layers were combined, washed with brine (1×5 mL), dried over anhydrous Na₂SO₄, and filtered. After filtration, the filtrate was concentrated under reduced pressure to give a residue. The residue was purified by reverse flash chromatography to yield tert-butyl 3-(5-fluoro-7-{8-fluoro-2-methylimidazo[1,2-a]pyridin-6-yl}-4-oxoquinazolin-3-yl)-4-oxopiperidine-1-carboxylate (C13, 13 mg, 32%) as a solid. LCMS (ES, m/z): 510 [M+H]⁺.

### Synthesis of Compound 352

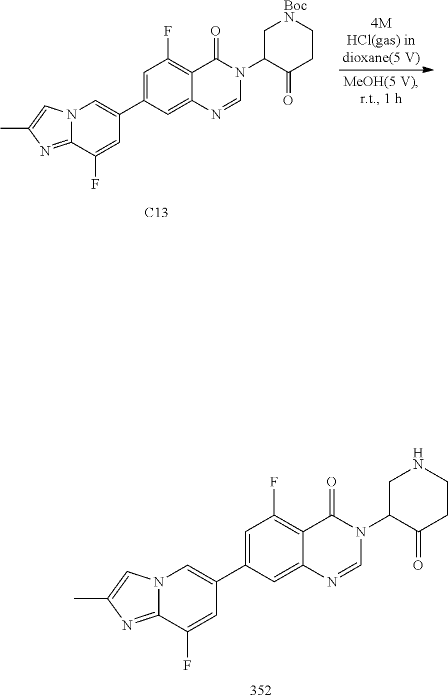 [SMILES: Cc1cn2cc(-c3cc(F)c4c(=O)n(C5CN(C(=O)OC(C)(C)C)CCC5=O)cnc4c3)cc(F)c2n1 ; "C13" ; Cc1cn2cc(-c3cc(F)c4c(=O)n(C5CNCCC5=O)cnc4c3)cc(F)c2n1 ; "352"]

A mixture of tert-butyl 3-(5-fluoro-7-{8-fluoro-2-methylimidazo[1,2-a]pyridin-6-yl}-4-oxoquinazolin-3-yl)-4-oxopiperidine-1-carboxylate (13.0 mg, 0.03 mmol, 1.00 equiv) in 4 M HCl (gas) in 1,4-dioxane (0.5 mL) and methanol (0.5 mL) was stirred for 1 hr at room temperature. The resulting mixture was concentrated under vacuum to give a residue. The residue was purified by Prep-HPLC (Condition 1, Gradient 4) to afford 5-fluoro-7-{8-fluoro-2-methylimidazo[1,2-a]pyridin-6-yl}-3-(4-oxopiperidin-3-yl)quinazolin-4-one (Compound 352, 5.5 mg, 51%) as a solid. LCMS (ES, m/z): 410 [M+H]^{+ 1}H NMR (400 MHz, DMSO-d₆) δ 9.09 (d, J=1.6 Hz, 1H), 8.33 (s, 1H), 7.93 (d, J=1.8 Hz, 1H), 7.84 (dd, J=3.2, 1.1 Hz, 1H), 7.77 (ddd, J=12.7, 8.4, 1.7 Hz, 2H), 5.42 (dd, J=10.8, 7.0 Hz, 1H), 3.47-3.39 (m, 1H), 3.28 (s, 1H), 2.95 (t, J=12.5 Hz, 2H), 2.70-2.59 (m, 1H), 2.44 (d, J=14.6 Hz, 1H), 2.39 (d, J=0.9 Hz, 3H). ¹⁹F NMR (376 MHz, DMSO) δ−109.76, −131.85.

### Example 95: Synthesis of Compound 343

### Synthesis of Intermediate C14

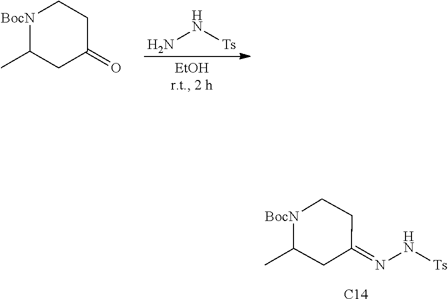 [SMILES: CC1CC(=O)CCN1C(=O)OC(C)(C)C ; Cc1ccc(S(=O)(=O)NN)cc1 ; Cc1ccc(S(=O)(=O)N/N=C2\CCN(C(=O)OC(C)(C)C)C(C)C2)cc1 ; "C14"]

A mixture of tert-butyl 2-methyl-4-oxopiperidine-1-carboxylate (1 g, 4.689 mmol, 1.00 equiv) and 4-toluenesulfonyl hydrazide (0.87 g, 4.689 mmol, 1 equiv) in ethanol (10 mL) was stirred for 2 hr at room temperature under nitrogen atmosphere. The resulting mixture was concentrated in vacuum to afford tert-butyl (4E)-2-methyl-4-[(4-methylbenzenesulfonamido)imino]piperidine-1-carboxylate (C14, 1.5 g) as a solid. LCMS (ES, m/z): 382 [M+H]⁺.

### Synthesis of Intermediate C15

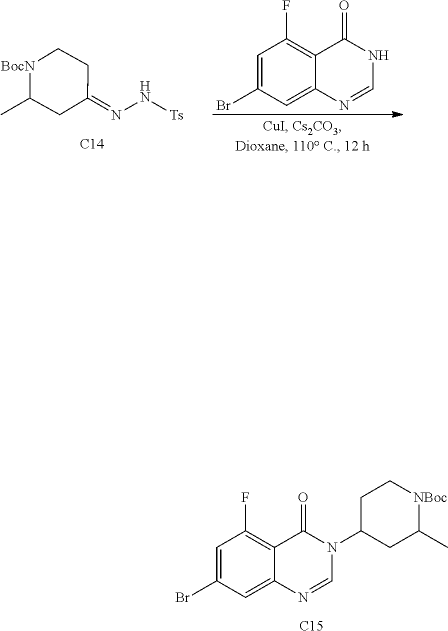 [SMILES: Cc1ccc(S(=O)(=O)N/N=C2\CCN(C(=O)OC(C)(C)C)C(C)C2)cc1 ; O=c1[nH]cnc2cc(Br)cc(F)c12 ; "C14" ; CC1CC(n2cnc3cc(Br)cc(F)c3c2=O)CCN1C(=O)OC(C)(C)C ; "C15"]

A mixture of tert-butyl (4E)-2-methyl-4-[(4-methylbenzenesulfonamido)imino]piperidine-1-carboxylate (600 mg, 1.573 mmol, 1.00 equiv), 7-bromo-5-fluoro-3H-quinazolin-4-one (573.36 mg, 2.359 mmol, 1.5 equiv), Cs₂CO₃ (1.5 g, 4.719 mmol, 3 equiv), and CuI (149 mg, 0.786 mmol, 0.5 equiv) in 1,4-dioxane (20 mL) was stirred for 12 hr at 110° C. under nitrogen atmosphere. The resulting mixture was concentrated under vacuum to give a residue. The residue was applied onto a silica gel column with ethyl acetate/hexane (1:1) to afford tert-butyl 4-(7-bromo-5-fluoro-4-oxoquinazolin-3-yl)-2-methylpiperidine-1-carboxylate (C15, 140 mg, 20%) as a solid. LCMS (ES, m/z): 462 [M+Na]⁺.

### Synthesis of Intermediate C16

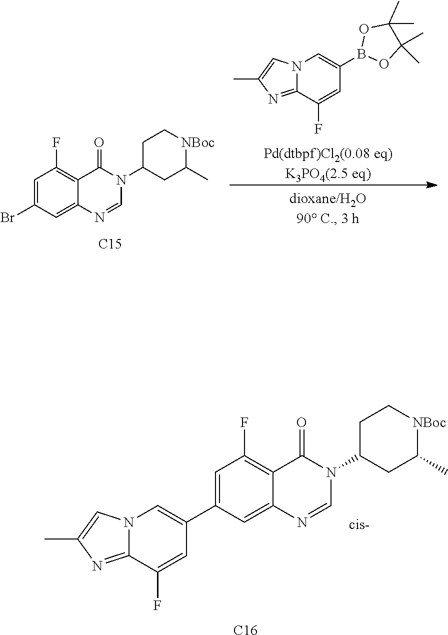

A mixture of tert-butyl 4-(7-bromo-5-fluoro-4-oxoquinazolin-3-yl)-2-methylpiperidine-1-carboxylate (140 mg, 0.318 mmol, 1.00 equiv), 8-fluoro-2-methyl-6-(4,4,5,5-tetramethyl-1,3,2-dioxaborolan-2-yl)imidazo[1,2-a]pyridine (131.69 mg, 0.477 mmol, 1.5 equiv), Pd(DtBPF)Cl₂ (20.72 mg, 0.032 mmol, 0.1 equiv), and K₃PO₄ (202.47 mg, 0.954 mmol, 3 equiv) in 1,4-dioxane (4 mL) and H₂O (1 mL) was stirred for 3 hr at 90° C. under nitrogen atmosphere. The resulting mixture was concentrated under reduced pressure to give a residue. The residue was applied onto a silica gel column with ethyl acetate/hexane (1:1), followed by chiral HPLC to afford cis-tert-butyl 4-(5-fluoro-7-{8-fluoro-2-methylimidazo[1,2-a]pyridin-6-yl}-4-oxoquinazolin-3-yl)-2-methylpiperidine-1-carboxylate (C16, 20 mg) as a solid. LCMS (ES, m/z): 510 [M+H]⁺.

### Synthesis of Compound 343

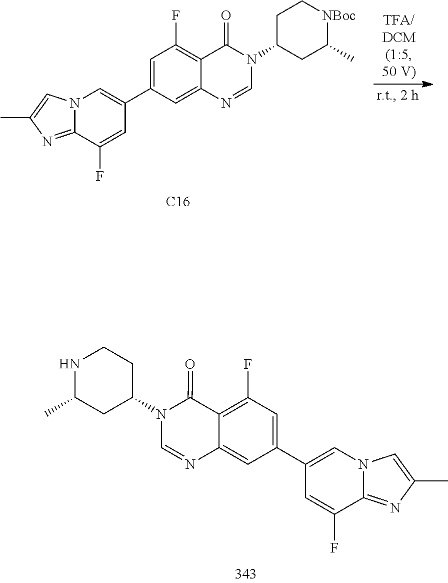

A mixture of cis-tert-butyl-4-(5-fluoro-7-{8-fluoro-2-methylimidazo[1,2-a]pyridin-6-yl}-4-oxoquinazolin-3-yl)-2-methylpiperidine-1-carboxylate (20 mg, 0.039 mmol, 1.00 equiv), dichloromethane (1.5 mL), and trifluoroacetic acid (0.25 mL) was stirred for 2 hr at room temperature under nitrogen atmosphere. The resulting mixture was concentrated to give a residue. The residue was purified by prep-HPLC (Condition 2, Gradient 3) to afford of cis-5-fluoro-7-(8-fluoro-2-methylimidazo[1,2-a]pyridin-6-yl)-3-(2-methylpiperidin-4-yl)quinazolin-4(3H)-one as a solid (Compound 343, 15 mg, 93%). LCMS (ES, m/z): 410 [M+H]^{+ 1}H NMR (400 MHz, Methanol-d₄) δ 9.25 (d, J=1.2 Hz, 1H), 8.97 (s, 1H), 8.34 (dd, J=10.8, 1.1 Hz, 1H), 8.23 (t, J=1.7 Hz, 1H), 7.97 (d, J=1.5 Hz, 1H), 7.85 (dd, J=11.6, 1.6 Hz, 1H), 5.02 (qd, J=8.2, 7.1, 4.3 Hz, 1H), 3.66 (ddd, J=13.0, 4.4, 2.1 Hz, 1H), 3.61-3.52 (m, 1H), 3.31 (d, J=10.1 Hz, 1H), 2.66 (s, 3H), 2.53 (qd, J=13.1, 4.5 Hz, 1H), 2.45-2.27 (m, 3H), 1.49 (d, J=6.4 Hz, 3H). ¹⁹F NMR (376 MHz, DMSO) δ 110.03, 133.52.

### Example 96: Synthesis of Compound 231

### Synthesis of Compound 231

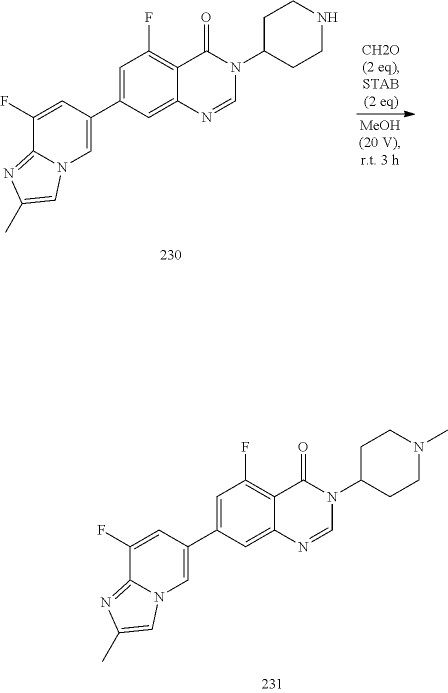 [SMILES: Cc1cn2cc(-c3cc(F)c4c(=O)n(C5CCNCC5)cnc4c3)cc(F)c2n1 ; Cc1cn2cc(-c3cc(F)c4c(=O)n(C5CCN(C)CC5)cnc4c3)cc(F)c2n1]

To a stirred mixture of 5-fluoro-7-{8-fluoro-2-methylimidazo[1,2-a]pyridin-6-yl}-3-(piperidin-4-yl) quinazolin-4-one (70.00 mg, 0.18 mmol, 1.00 equiv) and formic acid (10.63 mg, 0.35 mmol, 2.00 equiv) in methanol (3 mL) was stirred for 1 hr at room temperature. To this mixture was added sodium triacetoxyborohydride (75.04 mg, 0.35 mmol, 2.00 equiv). The resulting mixture was stirred for 2 hr at room temperature, then quenched with water (1 mL) at room temperature and extracted with ethyl acetate (3×10 mL). The organic layers were combined, washed with brine (1×10 mL), and dried over anhydrous Na₂SO₄, and filtered. After filtration, the filtrate was concentrated under reduced pressure to give a residue. The residue was purified by reverse flash chromatography (Condition 7, Gradient 1) to afford 5-fluoro-7-{8-fluoro-2-methylimidazo[1,2-a]pyridin-6-yl}-3-(1-methylpiperidin-4-yl)quinazolin-4-one (Compound 231, 10.70 mg, 14%) as a solid. LCMS (ES, m/z):410 [M+H]^{+ 1}H NMR (400 MHz, DMSO-d₆) δ 10.80 (s, 1H), 9.42 (d, J=1.4 Hz, 1H), 8.35 (s, 1H), 7.91-8.03 (m, 3H), 7.87 (dd, J=12.3, 1.8 Hz, 1H), 4.86 (ddt, J=12.4, 7.8, 4.0 Hz, 1H), 3.56 (d, J=11.8 Hz, 2H), 3.22 (dt, J=13.7, 10.7 Hz, 2H), 2.97 (d, J=5.1 Hz, 3H), 2.79 (d, J=4.7 Hz, 2H), 2.45 (d, J=4.5 Hz, 3H), 2.11 (d, J=12.8 Hz, 2H).

### Example 97: Synthesis of Compound 340

### Synthesis of Intermediate C17

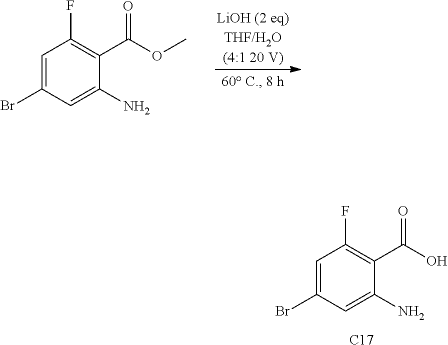

A mixture of methyl 2-amino-4-bromo-6-fluorobenzoate (500 mg, 2.016 mmol, 1.00 equiv), lithium hydroxide (96.54 mg, 4.032 mmol, 2 equiv), tetrahydrofuran (8 mL) and H₂O (2 mL) was stirred for 8 hr at 60° C. under nitrogen atmosphere. The reaction mixture was quenched with ice water (10 mL) and acidified with hydrochloric acid. The resulting solution was extracted with ethyl acetate (3×10 mL). The organic layers were combined, dried over anhydrous sodium sulfate, filtered, and the filtrate concentrated under reduced pressure to give a residue. The residue was applied onto a silica gel column with ethyl acetate/hexane (1:1) to afford 2-amino-4-bromo-6-fluorobenzoic acid (C17, 300 mg, 63%) as a solid. LCMS (ES, m/z): 234 [M+H]⁺.

### Synthesis of Intermediate C18

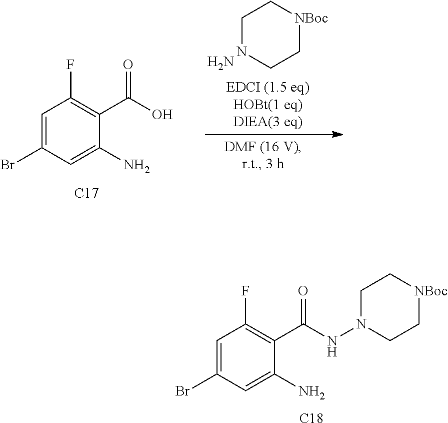

A mixture of 2-amino-4-bromo-6-fluorobenzoic acid (300 mg, 1.282 mmol, 1.00 equiv), tert-butyl 4-aminopiperazine-1-carboxylate (283.81 mg, 1.410 mmol, 1.1 equiv), EDCI (368.62 mg, 1.923 mmol, 1.5 equiv), and HOBT (173.22 mg, 1.282 mmol, 1 equiv) in dimethylformamide (5 mL, 64.609 mmol, 50.40 equiv) was stirred for 3 hr at room temperature under nitrogen atmosphere. The reaction was quenched with water (20 mL) and extracted with ethyl acetate (3×20 mL). The organic layers were combined, dried over anhydrous sodium sulfate, filtered, and the filtrate concentrated under reduce pressure to give a residue. The residue was applied onto a silica gel column with ethyl acetate/hexane (1:1) to afford tert-butyl 4-(2-amino-4-bromo-6-fluorobenzamido)piperazine-1-carboxylate (C18, 250 mg, 46%) as a solid. LCMS (ES, m/z): 417 [M+H]⁺.

### Synthesis of Intermediate C19

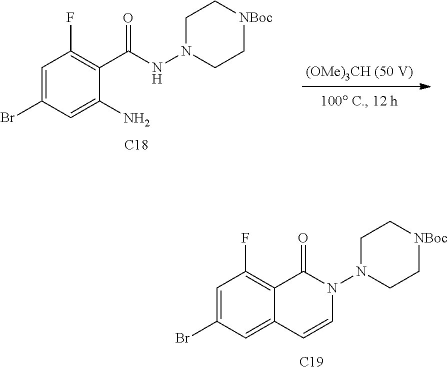 [SMILES: CC(C)(C)OC(=O)N1CCN(NC(=O)c2c(N)cc(Br)cc2F)CC1 ; "C18" ; CC(C)(C)OC(=O)N1CCN(n2ccc3cc(Br)cc(F)c3c2=O)CC1 ; "C19"]

A mixture of tert-butyl 4-(2-amino-4-bromo-6-fluorobenzamido)piperazine-1-carboxylate (100 mg, 0.240 mmol, 1.00 equiv) in trimethyl orthoformate (5 mL) was heated to 100° C. for 12 h, then concentrated in vacuo to give a residue. The residue was applied onto a silica gel column with ethyl acetate/hexane (1:2) to afford tert-butyl 4-(7-bromo-5-fluoro-4-oxoquinazolin-3-yl)piperazine-1-carboxylate (C19, 70 mg, 68%) as a solid. LCMS (ES, m/z): 429 [M+H]⁺.

### Synthesis of Intermediate C20

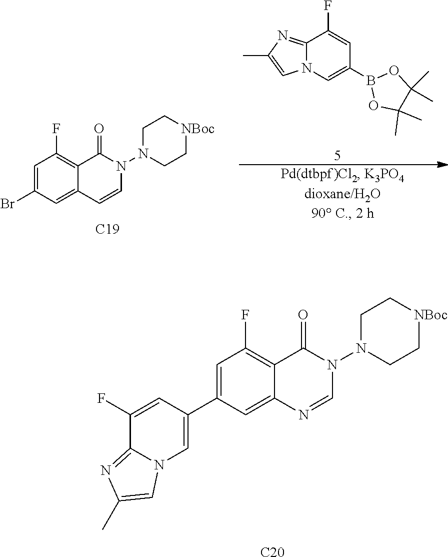

A mixture of tert-butyl 4-(7-bromo-5-fluoro-4-oxoquinazolin-3-yl)piperazine-1-carboxylate (70 mg, 0.164 mmol, 1.00 equiv), 8-fluoro-2-methyl-6-(4,4,5,5-tetramethyl-1,3,2-dioxaborolan-2-yl)imidazo[1,2-a]pyridine (50 mg, 0.180 mmol, 1.1 equiv), Pd(dtbpf)Cl₂ (10.64 mg, 0.016 mmol, 0.1 equiv), K₃PO₄ (104.33 mg, 0.492 mmol, 3 equiv),1,4-dioxane (2 mL), and H₂O (0.5 mL) was stirred for 2 hr at 80° C. The resulting mixture was concentrated to give a residue. The residue was applied onto a silica gel column with ethyl acetate/hexane (1:1) to afford tert-butyl 4-(5-fluoro-7-{8-fluoro-2-methylimidazo[1,2-a]pyridin-6-yl}-4-oxoquinazolin-3-yl)piperazine-1-carboxylate (C20, 50 mg, 61%) as a solid. LCMS (ES, m/z): 497 [M+H]⁺.

### Synthesis of Compound 340

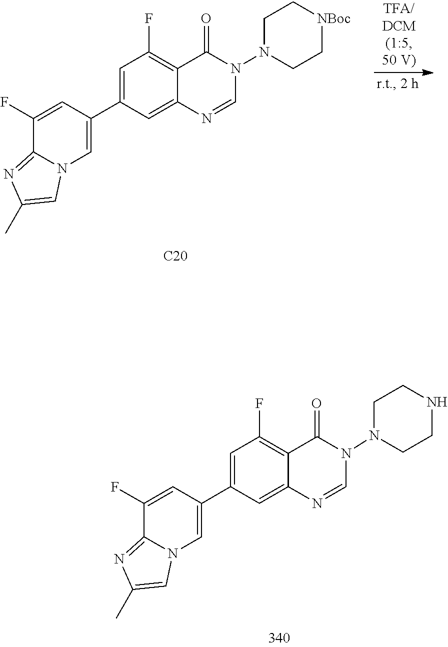 [SMILES: Cc1cn2cc(-c3cc(F)c4c(=O)n(N5CCN(C(=O)OC(C)(C)C)CC5)cnc4c3)cc(F)c2n1 ; "C20" ; Cc1cn2cc(-c3cc(F)c4c(=O)n(N5CCNCC5)cnc4c3)cc(F)c2n1]

A mixture of tert-butyl 4-(5-fluoro-7-{8-fluoro-2-methylimidazo[1,2-a]pyridin-6-yl}-4-oxoquinazolin-3-yl)piperazine-1-carboxylate (40 mg, 0.081 mmol, 1.00 equiv), TFA (0.5 mL), and DCM (2.5 mL) was stirred for 1 h at 25° C. The resulting mixture was concentrated to give a residue. The residue was purified by Prep-HPLC (Condition 3, Gradient 1) to afford 5-fluoro-7-{8-fluoro-2-methylimidazo[1,2-a]pyridin-6-yl}-3-(piperazin-1-yl)quinazolin-4-one (Compound 340, 15 mg, 46%) as a solid. LCMS (ES, m/z): 397 [M+H]^{+ 1}H NMR (400 MHz, DMSO-d₆) δ 9.34 (s, 1H), 9.18-8.58 (m, 2H), 8.28 (s, 1H), 8.24 (d, J=12.3 Hz, 1H), 8.06 (d, J=2.5 Hz, 1H), 8.00 (d, J=1.7 Hz, 1H), 7.88 (d, J=1.8 Hz, 1H), 7.85 (d, J=1.8 Hz, 1H), 3.37-2.88 (m, 8H), 2.51 (s, 3H). ¹⁹F NMR (376 MHz, DMSO) δ−109.81, 131.65.

### Example 98: Synthesis of Compound 353

### Synthesis of Intermediate C21

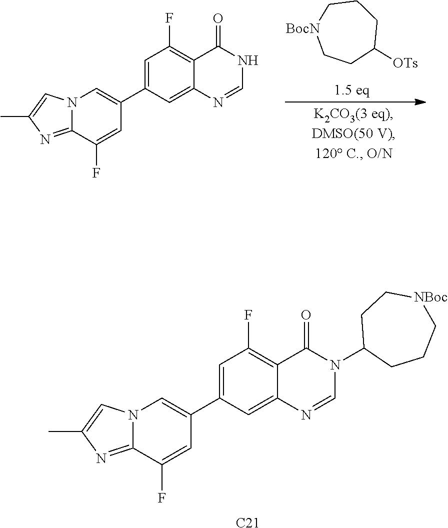 [SMILES: Cc1cn2cc(-c3cc(F)c4c(=O)[nH]cnc4c3)cc(F)c2n1 ; Cc1ccc(S(=O)(=O)OC2CCCN(C(=O)OC(C)(C)C)CC2)cc1 ; Cc1cn2cc(-c3cc(F)c4c(=O)n(C5CCCN(C(=O)OC(C)(C)C)CC5)cnc4c3)cc(F)c2n1 ; "C21"]

A mixture of 5-fluoro-7-{8-fluoro-2-methylimidazo[1,2-a]pyridin-6-yl}-3H-quinazolin-4-one (250.0 mg, 0.79 mmol, 1.00 equiv), tert-butyl 4-[(4-methylbenzenesulfonyl)oxy]azepane-1-carboxylate (442.8 mg, 1.18 mmol, 1.5 equiv), and K₂CO₃ (331.3 mg, 2.39 mmol, 3 equiv) in DMSO (10 mL) was stirred overnight at 120° C. The solution was purified by reverse flash chromatography (Condition 3, Gradient 2) to yield tert-butyl 4-(5-fluoro-7-{8-fluoro-2-methylimidazo[1,2-a]pyridin-6-yl}-4-oxoquinazolin-3-yl)azepane-1-carboxylate (C21, 60 mg, 14%) as a solid. LCMS (ES, m/z): 510 [M+H]⁺.

### Synthesis of Compound 353

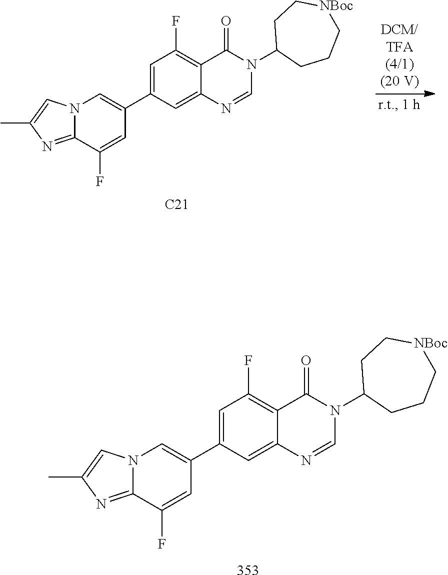 [SMILES: Cc1cn2cc(-c3cc(F)c4c(=O)n(C5CCCN(C(=O)OC(C)(C)C)CC5)cnc4c3)cc(F)c2n1 ; "C21" ; Cc1cn2cc(-c3cc(F)c4c(=O)n(C5CCCN(C(=O)OC(C)(C)C)CC5)cnc4c3)cc(F)c2n1 ; "353"]

A solution tert-butyl 4-(5-fluoro-7-{8-fluoro-2-methylimidazo[1,2-a]pyridin-6-yl}-4-oxoquinazolin-3-yl)azepane-1-carboxylate (60 mg, 0.11 mmol, 1.00 equiv) in trifluoroacetic acid (0.2 mL) and dichloromethane (0.8 mL) was stirred for 1 hr at room temperature. The resulting mixture was concentrated under vacuum to give a residue. The residue was purified by Prep-HPLC (Condition 3, Gradient 3) to afford 3-(azepan-4-yl)-5-fluoro-7-{8-fluoro-2-methylimidazo[1,2-a]pyridin-6-yl}quinazolin-4-one (Compound 353, 12.4 mg, 26%) as a solid. LCMS (ES, m/z): 410 [M+H]^{+ 1}H NMR (400 MHz, DMSO-d₆) δ 9.48-9.23 (m, 2H), 9.15 (s, 1H), 8.54 (s, 1H), 8.34 (d, J=11.8 Hz, 1H), 8.10 (d, J=2.5 Hz, 1H), 7.99 (d, J=1.8 Hz, 1H), 7.84 (dd, J=12.2, 1.9 Hz, 1H), 4.83 (d, J=12.3 Hz, 1H), 3.38 (d, J=7.3 Hz, 1H), 3.18 (dd, J=22.4, 11.8 Hz, 3H), 2.48 (s, 3H), 2.33 (p, J=1.8 Hz, 1H), 2.22 (dt, J=27.8, 13.5 Hz, 2H), 2.06-1.94 (m, 2H), 1.87 (t, J=10.4 Hz, 1H). ¹⁹F NMR (376 MHz, DMSO) δ−109.70, −132.10.

### Example 99: Synthesis of Compounds 349 and 350

### Synthesis of Intermediate C22

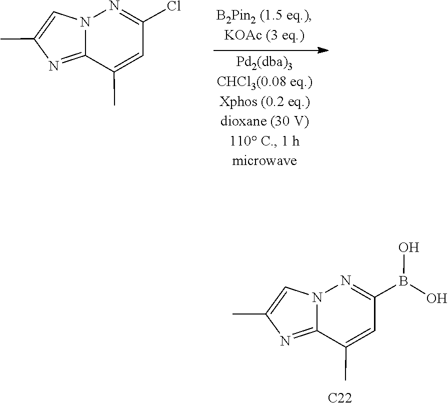

A mixture of 6-chloro-2,8-dimethylimidazo[1,2-b]pyridazine (500 mg, 2.75 mmol, 1.00 equiv), 4,4,5,5-tetramethyl-2-(tetramethyl-1,3,2-dioxaborolan-2-yl)-1,3,2-dioxaborolane (1.05 g, 4.13 mmol, 1.5 equiv), potassium acetate (810.56 mg, 8.26 mmol, 3.00 equiv), XPhos (262.48 mg, 0.55 mmol, 0.20 equiv), Pd₂(dba)₃CHCl₃ (227.97 mg, 0.22 mmol, 0.08 equiv) and dioxane (15 mL) was evacuated and flushed three times with nitrogen. The reaction mixture was irradiated with microwave radiation for 1 hr at 110° C., then filtered to remove solids. LCMS (ES, m/z): 192 [M+H]⁺.

### Synthesis of Intermediate C23

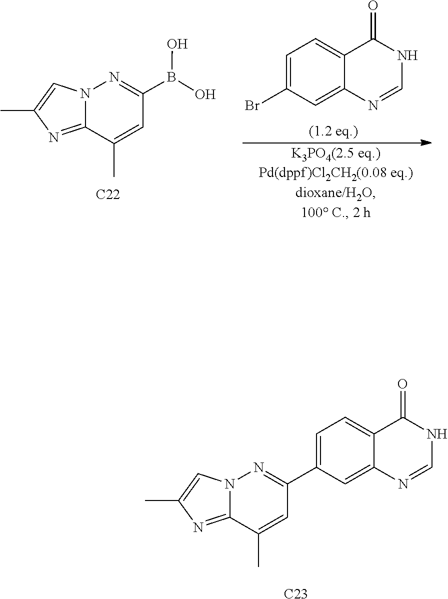 [SMILES: Cc1cn2nc(B(O)O)cc(C)c2n1 ; O=c1[nH]cnc2cc(Br)ccc12 ; "C22" ; Cc1cn2nc(-c3ccc4c(=O)[nH]cnc4c3)cc(C)c2n1 ; "C23"]

A mixture of 2,8-dimethylimidazo[1,2-b]pyridazin-6-ylboronic acid (500 mg, 2.62 mmol, 1.00 equiv), 7-bromo-3H-quinazolin-4-one (706.66 mg, 3.14 mmol, 1.2 equiv), dioxane (15 mL), K₃PO₄ (1.38 g, 6.54 mmol, 2.50 equiv), H₂O (3 mL) and Pd(dppf)Cl₂·CH₂Cl₂ (170.60 mg, 0.209 mmol, 0.08 equiv) was evacuated and flushed three times with nitrogen. The resulting mixture was stirred for 2 hr at 100° C., then quenched with water (40 mL). The reaction mixture was filtered and the filter cake washed with hexane (20 ml). The filtrate was concentrated under vacuum to afford 7-{2,8-dimethylimidazo-[1,2-b]pyridazin-6-yl}-3H-quinazolin-4-one (700 mg) as a solid. LCMS (ES, m/z):292 [M+H]⁺

### Synthesis of Intermediate C24

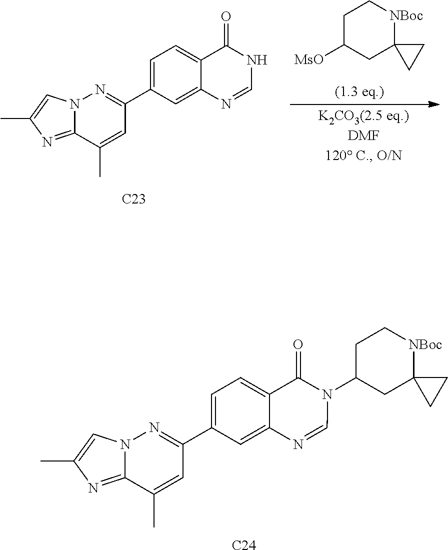 [SMILES: Cc1cn2nc(-c3ccc4c(=O)[nH]cnc4c3)cc(C)c2n1 ; CC(C)(C)OC(=O)N1CCC(OS(C)(=O)=O)CC12CC2 ; "C23" ; Cc1cn2nc(-c3ccc4c(=O)n(C5CCN(C(=O)OC(C)(C)C)C6(CC6)C5)cnc4c3)cc(C)c2n1 ; "C24"]

A mixture of 7-{2,8-dimethylimidazo[1,2-b]pyridazin-6-yl}-3H-quinazolin-4-one (350 mg, 1.20 mmol, 1.00 equiv), tert-butyl 7-(methanesulfonyloxy)-4-azaspiro[2.5]octane-4-carboxylate (476.89 mg, 1.56 mmol, 1.3 equiv), K₂CO₃ (414.95 mg, 3.00 mmol, 2.5 equiv), and dimethylformamide (12 mL) was stirred overnight at 120° C., then cooled to room temperature. The reaction mixture was quenched with water (40 mL), then filtered and the filtrate concentrated under vacuum to give a residue. The residue was purified by Prep-HPLC (Condition 2, Gradient 4) to afford tert-butyl 7-(7-{2,8-dimethylimidazo[1,2-b]pyridazin-6-yl}-4-oxoquinazolin-3-yl)-4-azaspiro[2.5]octane-4-carboxylate (C24, 40 mg, 6%) as a solid. LCMS (ES, m/z): 501 [M+H]⁺.

### Synthesis of Compound 349 and 350

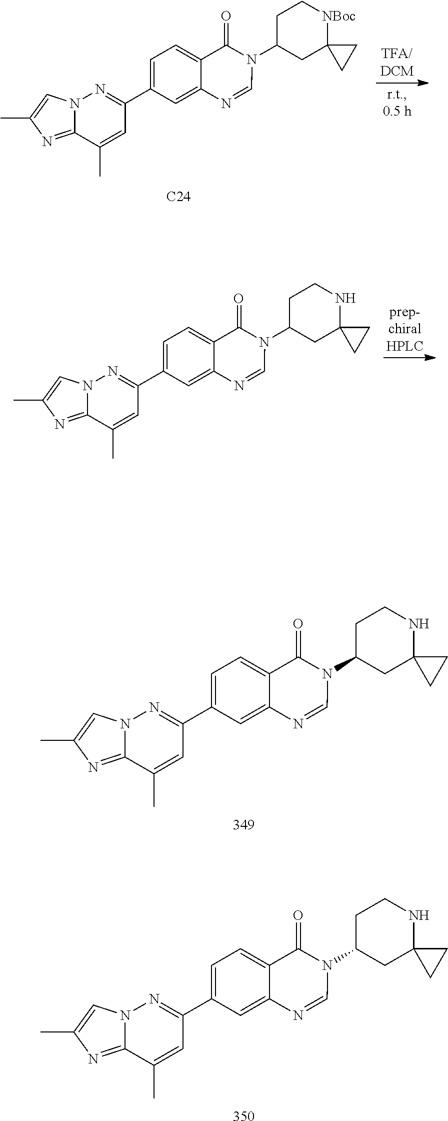

A mixture of tert-butyl 7-(7-{2,8-dimethylimidazo[1,2-b]pyridazin-6-yl}-4-oxoquinazolin-3-yl)-4-azaspiro[2.5]octane-4-carboxylate (40 mg, 0.08 mmol, 1.00 equiv), dichloromethane (2 mL), and trifluoroacetic acid (0.4 mL) was stirred for 30 min at room temperature. The resulting mixture was concentrated under vacuum to give a residue. The residue was purified by Chiral-Prep-HPLC (Condition 1) to afford 3-[(7S)-4-azaspiro[2.5]octan-7-yl]-7-{2,8-dimethylimidazo[1,2-b]pyridazin-6-yl}quinazolin-4-one (Compound 349, 11 mg) as a solid and 3-[(7R)-4-azaspiro[2.5]octan-7-yl]-7-{2,8-dimethylimidazo[1,2-b]pyridazin-6-yl}quinazolin-4-one (Compound 350, 10.8 mg) as a solid.

| Compound | |
|---|---|
| No. | Analysis Data |
| 349 | LCMS: (ES, m/z): 401 [M + H]⁺ |
| | ¹H-NMR: (400 MHz, DMSO-d₆) δ 8.51 (s, 1H), 8.33 (d, J = 1.7 Hz, 1H), |
| | 8.30 (d, J = 8.4 Hz, 1H), 8.23 (dd, J = 8.4, 1.8 Hz, 1H), 8.14 (d, J = 1.0 Hz, |
| | 1H), 7.85 (d, J = 1.2 Hz, 1H), 5.16-4.49 (m, 1H), 3.11 (d, J = 12.6 Hz, |
| | 1H), 2.81 (m, 1H), 2.64 (d, J = 1.0 Hz, 3H), 2.48-2.27 (m, 3H), 2.00 |
| | (s, 1H), 1.91 (s, 1H), 1.35-1.23 (m, 1H), 1.20-1.11 (m, 1H), 0.66 (s, 1H), |
| | 0.53 (s, 2H), 0.45 (d, J = 7.7 Hz, 1H). |
| 350 | LCMS: (ES, m/z): 401 [M + H]⁺ |
| | ¹H-NMR: (400 MHz, DMSO-d₆) δ 8.51 (s, 1H), 8.33 (d, J = 1.7 Hz, 1H), |
| | 8.30 (d, J = 8.4 Hz, 1H), 8.23 (dd, J = 8.4, 1.8 Hz, 1H), 8.14 (d, J = 1.0 Hz, |
| | 1H), 7.85 (d, J = 1.2 Hz, 1H), 5.16-4.49 (m, 1H), 3.11 (d, J = 12.6 Hz, |
| | 1H), 2.81 (m, 1H), 2.64 (d, J = 1.0 Hz, 3H), 2.48-2.27 (m, 3H), 2.00 |
| | (s, 1H), 1.91 (s, 1H), 1.35-1.23 (m, 1H), 1.20-1.11 (m, 1H), 0.66 (s, 1H), |
| | 0.53 (s, 2H), 0.45 (d, J = 7.7 Hz, 1H). |

### Example 100: Synthesis of Compound 381

### Synthesis of Intermediate C25

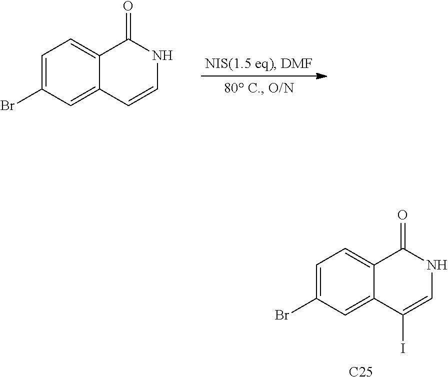 [SMILES: O=c1[nH]ccc2cc(Br)ccc12 ; O=c1[nH]cc(I)c2cc(Br)ccc12 ; "C25"]

A solution of 6-bromo-2H-isoquinolin-1-one (1.0 g, 4.463 mmol, 1.00 equiv) and NIS (1.5 g, 6.71 mmol, 1.50 equiv) in DMF (10 ml) was stirred overnight at 80° C. A precipitate formed that was collected by filtration and washed with H₂O (3×10 mL) to afford 6-bromo-4-iodo-2H-isoquinolin-1-one (C25, 1.3 g, 83%) as a solid. LCMS (ES, m/z): 350 [M+H]⁺.

### Synthesis of Intermediate C26

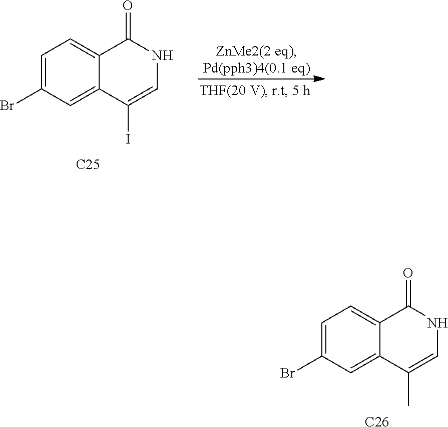

To a stirred mixture of 6-bromo-4-iodo-2H-isoquinolin-1-one (1.3 g, 3.715 mmol, 1 equiv) and Pd(PPh₃)₄ (0.43 g, 0.37 mmol, 0.10 equiv) in tetrahydrofuran (20 mL) was added Zn(CH₃)₂ (0.71 g, 7.43 mmol, 2.00 equiv). The reaction mixture was stirred for 5 hr at room temperature, then quenched with AcOH (1 M). A precipitate formed that was collected by filtration and washed with H₂O (3×10 mL) to afford 6-bromo-4-methyl-2H-isoquinolin-1-one (C26, 750 mg, 84%) as a solid. LCMS (ES, m/z): 238 [M+H]⁺.

### Synthesis of Intermediate C27

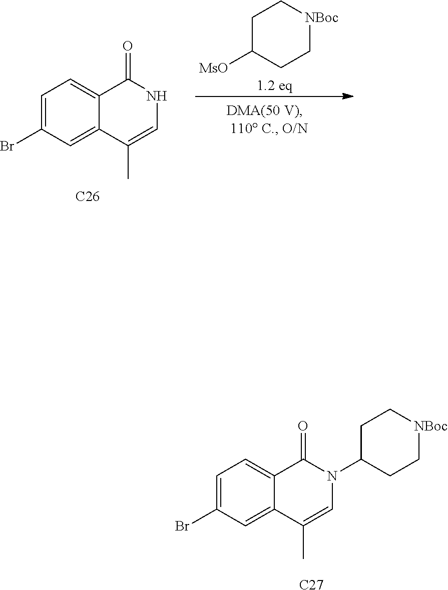 [SMILES: Cc1c[nH]c(=O)c2ccc(Br)cc12 ; CC(C)(C)OC(=O)N1CCC(OS(C)(=O)=O)CC1 ; "C26" ; Cc1cn(C2CCN(C(=O)OC(C)(C)C)CC2)c(=O)c2ccc(Br)cc12 ; "C27"]

To a stirred mixture of 6-bromo-4-methyl-2H-isoquinolin-1-one (700 mg, 2.94 mmol, 1.00 equiv) and tert-butyl 4-(methanesulfonyloxy)piperidine-1-carboxylate (985.59 mg, 3.53 mmol, 1.20 equiv) in dimethylacetamide (10 mL) was added Cs₂CO₃ (2873.87 mg, 8.82 mmol, 3.00 equiv) in portions at 110° C. The resulting mixture was extracted with ethyl acetate (3×10 mL). The organic layers were combined, washed with saturated NaCl (2×10 mL), dried over anhydrous Na₂SO₄, and filtered. After filtration, the filtrate was concentrated under reduced pressure to give a residue. The residue was purified by silica gel column chromatography, eluted with PE/EA (5:1) to afford tert-butyl 4-(6-bromo-4-methyl-1-oxoisoquinolin-2-yl)piperidine-1-carboxylate (C27, 220 mg, 17%) as a solid. LCMS (ES, m/z): 421[M+H]⁺.

### Synthesis of Intermediate C28

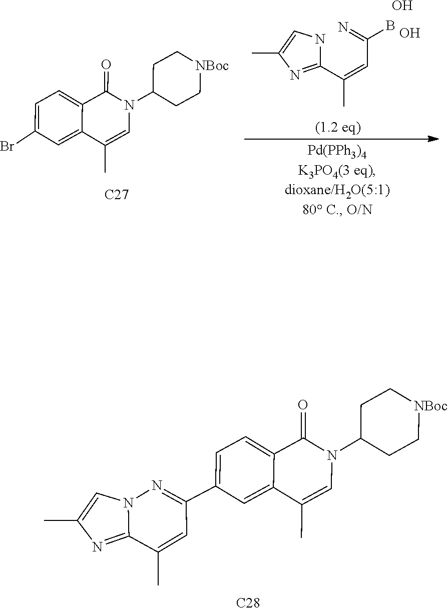

To a stirred mixture of tert-butyl 4-(6-bromo-4-methyl-1-oxoisoquinolin-2-yl)piperidine-1-carboxylate (220 mg, 0.52 mmol, 1.00 equiv) and 2,8-dimethylimidazo[1,2-b]pyridazin-6-ylboronic acid (119.68 mg, 0.63 mmol, 1.20 equiv) in dioxane/H₂O (4:1, 5 mL) was added K₃PO₄ (332.50 mg, 1.57 mmol, 3.00 equiv) and Pd(PPh₃)₄ (60.34 mg, 0.05 mmol, 0.10 equiv). The reaction mixture was stirred overnight at 80° C. under a nitrogen atmosphere. The resulting mixture was extracted with ethyl acetate (3×10 mL). The organic layers were combined, washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (5:1) to afford tert-butyl 4-(6-{2,8-dimethylimidazo[1,2-b]pyridazin-6-yl}-4-methyl-1-oxoisoquinolin-2-yl)piperidine-1-carboxylate (C28, 110 mg, 43%) as a solid. LCMS (ES, m/z):488 [M+H]⁺.

### Synthesis of Compound 381

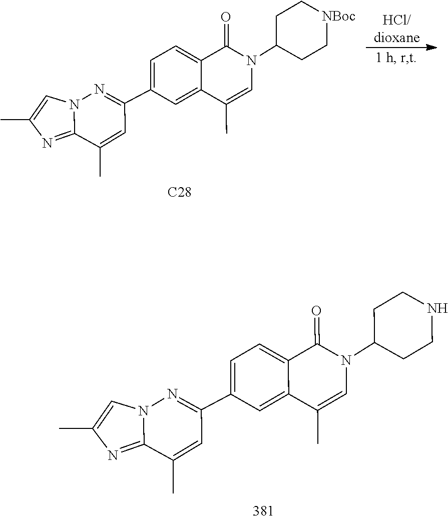 [SMILES: Cc1cn2nc(-c3ccc4c(=O)n(C5CCN(C(=O)OC(C)(C)C)CC5)cc(C)c4c3)cc(C)c2n1 ; "C28" ; Cc1cn2nc(-c3ccc4c(=O)n(C5CCNCC5)cc(C)c4c3)cc(C)c2n1 ; "381"]

A solution of tert-butyl 4-(6-{2,8-dimethylimidazo[1,2-b]pyridazin-6-yl}-4-methyl-1-oxoisoquinolin-2-yl)piperidine-1-carboxylate (110 mg, 0.23 mmol, 1.00 equiv) in HCl (gas) in 1,4-dioxane (10 mL) was stirred for 1 hr at room temperature. The resulting mixture was concentrated under reduced pressure to give a residue. The residue was purified by reverse flash chromatography (Condition 8, Gradient 1) to afford 6-{2,8-dimethylimidazo[1,2-b]pyridazin-6-yl}-4-methyl-2-(piperidin-4-yl)isoquinolin-1-one (Compound 381, 30.70 mg, 35%) as a solid. LCMS (ES, m/z):388 [M+H]^{+ 1}H NMR (400 MHz, DMSO-d₆) δ 8.41 (d, J=8.5 Hz, 1H), 8.19 (d, J=1.8 Hz, 1H), 8.11 (dd, J=8.4, 1.8 Hz, 1H), 8.01 (d, J=1.0 Hz, 1H), 7.67 (d, J=1.2 Hz, 1H), 7.32 (d, J=1.4 Hz, 1H), 4.87 (tt, J=12.0, 4.1 Hz, 1H), 3.13-3.05 (m, 2H), 2.69-2.58 (m, 5H), 2.42 (d, J=0.9 Hz, 3H), 2.37 (d, J=1.2 Hz, 3H), 1.88-1.77 (m, 2H), 1.76-1.66 (m, 2H).

### Example 101: Synthesis of Compounds 397 and 398

### Synthesis of Intermediate C29

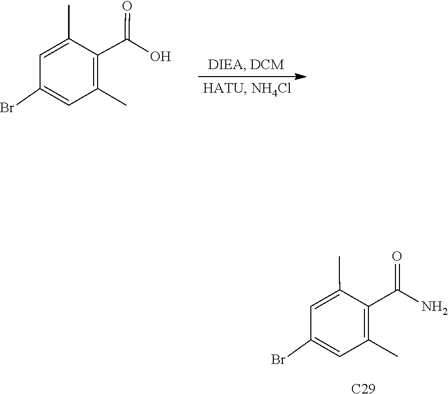

A mixture of 4-bromo-2,6-dimethylbenzoic acid (4 g, 17.462 mmol, 1.0 equiv), DCM (40 mL), HATU (7.97 g, 20.954 mmol, 1.2 equiv), DIEA (7.07 g, 69.848 mmol, 4.0 equiv) and NH₄Cl (2.80 g, 52.386 mmol, 3.0 equiv) was stirred for 4 hr at room temperature, then diluted with water (100 mL). The resulting mixture was extracted with CH₂Cl₂ (3×100 mL). The organic layers were combined, washed with brine (1×100 mL), dried over anhydrous Na₂SO₄, and filtered. After filtration, the filtrate was concentrated under reduced pressure to give a residue. The residue was purified by silica gel column chromatography, eluted with PE/EA (1:1) to afford 4-bromo-2,6-dimethylbenzamide (C29, 3.6 g, 9%) as a solid. LCMS (ES, m/z): 228 [M+H]⁺.

### Synthesis of Intermediate C30

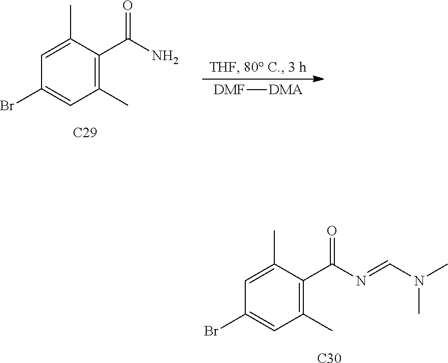

A mixture of 4-bromo-2,6-dimethylbenzamide (3.0 g, 13.153 mmol, 1.0 equiv), tetrahydrofuran (15 mL) and dimethylformamide-dimethyl acetal (1.72 g, 14.468 mmol, 1.1 equiv) was stirred for 1 hr at 80° C. The mixture was concentrated under reduced pressure to give a solid. The solid was triturated with hexane to afford 4-bromo-N-[(1E)-(dimethylamino)methylidene]-2,6-dimethylbenzamide (C30, 3.0 g, 80%) as a solid. LCMS (ES, m/z): 283 [M+H]⁺

### Synthesis of Compound C₃₁

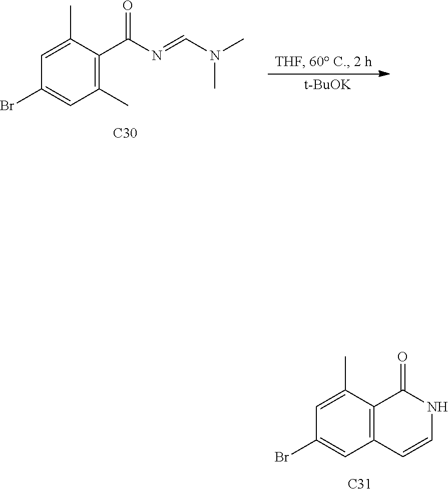 [SMILES: "t-BuOK" ; Cc1cc(Br)cc(C)c1C(=O)/N=C/N(C)C ; "C30" ; Cc1cc(Br)cc2cc[nH]c(=O)c12 ; "C31"]

To a mixture of 4-bromo-N-[(1E)-(dimethylamino)methylidene]-2,6-dimethylbenzamide (4.0 g, 14.126 mmol, 1 equiv) in THF (8 mL) was added potassium tert-butoxide in THF (1 M) (26.66 mL, 237.599 mmol, 16.82 equiv) dropwise at 60° C. The resulting mixture was stirred for 2 hr at 60° C., then concentrated under reduced pressure to give a residue. The residue was acidified to pH 4 with 1 M HCl. The resulting mixture was filtered, washed with water (3×50 mL), and hexane (50 mL) to afford 6-bromo-8-methyl-2H-isoquinolin-1-one (C31, 3.0 g, 89%) as a solid. LCMS (ES, m/z): 238 [M+H]⁺.

### Synthesis of Intermediate C32

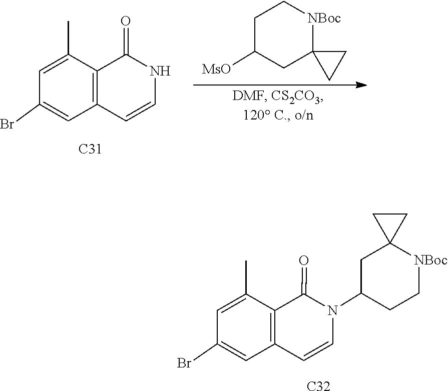 [SMILES: Cc1cc(Br)cc2cc[nH]c(=O)c12 ; CC(C)(C)OC(=O)N1CCC(OS(C)(=O)=O)CC12CC2 ; "C31" ; Cc1cc(Br)cc2ccn(C3CCN(C(=O)OC(C)(C)C)C4(CC4)C3)c(=O)c12 ; "C32"]

A mixture of 6-bromo-8-methyl-2H-isoquinolin-1-one (500 mg, 2.100 mmol, 1.0 equiv), DMF (5 mL), tert-butyl 7-(methanesulfonyloxy)-4-azaspiro[2.5]octane-4-carboxylate (962.02 mg, 3.150 mmol, 1.5 equiv) and Cs₂CO₃ (2052.76 mg, 6.300 mmol, 3.0 equiv) was stirred overnight at 120° C. The resulting mixture was diluted with water (40 mL) and extracted with ethyl acetate (3×40 mL). The organic layers were combined, washed with water (3×100 mL), dried over anhydrous Na₂SO₄, filtered, and the filtrate concentrated under reduced pressure to give a residue. The residue was purified by reverse phase flash chromatography to afford tert-butyl 7-(6-bromo-8-methyl-1-oxoisoquinolin-2-yl)-4-azaspiro[2.5]octane-4-carboxylate (C32, 220 mg, 23%) as a solid. LCMS (ES, m/z): 447 [M+H]⁺.

### Synthesis of Intermediate C33

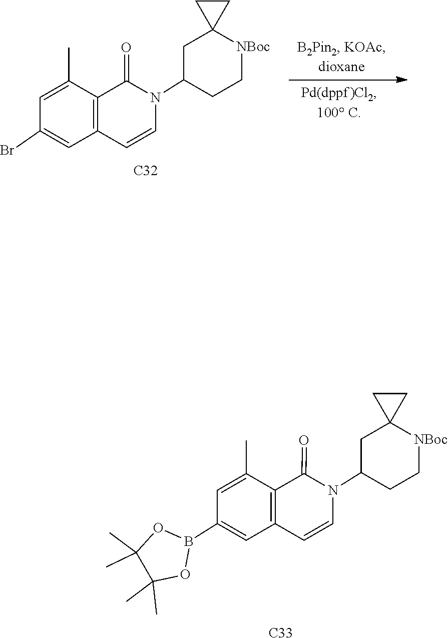 [SMILES: Cc1cc(Br)cc2ccn(C3CCN(C(=O)OC(C)(C)C)C4(CC4)C3)c(=O)c12 ; "C32" ; Cc1cc(B2OC(C)(C)C(C)(C)O2)cc2ccn(C3CCN(C(=O)OC(C)(C)C)C4(CC4)C3)c(=O)c12 ; "C33"]

A mixture of tert-butyl 7-(6-bromo-8-methyl-1-oxoisoquinolin-2-yl)-4-azaspiro[2.5]octane-4-carboxylate (220 mg, 0.492 mmol, 1.0 equiv), dioxane (3 mL), bis(pinacolato)diboron (149.85 mg, 0.590 mmol, 1.2 equiv), potassium acetate (144.79 mg, 1.476 mmol, 3.0 equiv), and Pd(dppf)Cl₂CH₂Cl₂ (40.06 mg, 0.049 mmol, 0.1 equiv) was stirred for 4 hr at 100° C. under a nitrogen atmosphere. LCMS (ES, m/z): 495 [M+H]⁺.

### Synthesis of Intermediate C34

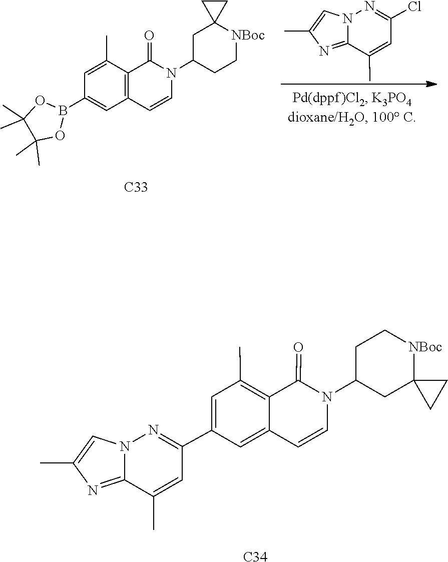 [SMILES: Cc1cc(B2OC(C)(C)C(C)(C)O2)cc2ccn(C3CCN(C(=O)OC(C)(C)C)C4(CC4)C3)c(=O)c12 ; Cc1cn2nc(Cl)cc(C)c2n1 ; "C33" ; Cc1cn2nc(-c3cc(C)c4c(=O)n(C5CCN(C(=O)OC(C)(C)C)C6(CC6)C5)ccc4c3)cc(C)c2n1 ; "C34"]

To the stirred mixture of C₃₃ was added water (0.5 mL), 6-chloro-2,8-dimethylimidazo[1,2-b]pyridazine (89.26 mg, 0.491 mmol, 1.0 equiv), K₃PO₄ (104.32 mg, 0.491 mmol, 1.0 equiv), and Pd(dppf)Cl₂CH₂Cl₂ (40.04 mg, 0.049 mmol, 0.1 equiv) at room temperature under a nitrogen atmosphere. The resulting mixture was stirred for 6 hr at 100° C. under nitrogen atmosphere, then diluted with water (20 mL) and extracted with ethyl acetate (3×40 mL). The organic layers were combined, washed with brine (1×100 mL), dried over anhydrous Na₂SO₄, filtered, and the filtrate concentrated under reduced pressure to give a residue. The residue was purified by silica gel column chromatography, eluted with CH₂Cl₂/MeOH (8:1) to afford tert-butyl 7-(6-{2,8-dimethylimidazo[1,2-b]pyridazin-6-yl}-8-methyl-1-oxoisoquinolin-2-yl)-4-azaspiro[2.5]octane-4-carboxylate (C34, 245 mg, 9%) as an oil. LCMS (ES, m/z): 514 [M+H]⁺.

### Synthesis of Intermediate C35

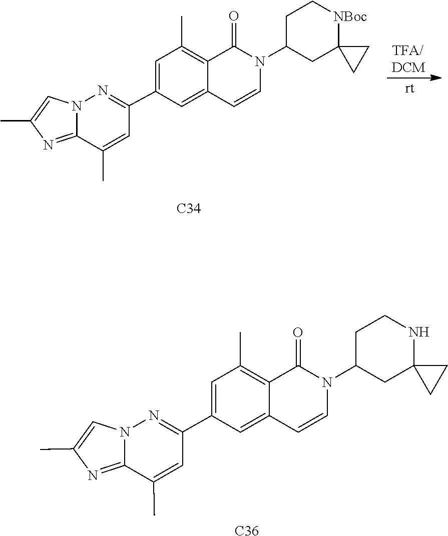 [SMILES: Cc1cn2nc(-c3cc(C)c4c(=O)n(C5CCN(C(=O)OC(C)(C)C)C6(CC6)C5)ccc4c3)cc(C)c2n1 ; "C34" ; Cc1cn2nc(-c3cc(C)c4c(=O)n(C5CCNC6(CC6)C5)ccc4c3)cc(C)c2n1 ; "C36"]

A mixture of tert-butyl 7-(6-{2,8-dimethylimidazo[1,2-b]pyridazin-6-yl}-8-methyl-1-oxoisoquinolin-2-yl)-4-azaspiro[2.5]octane-4-carboxylate (300 mg, 0.584 mmol, 1 equiv), DCM (3 mL), and TFA (3 mL, 40.389 mmol, 69.15 equiv) was stirred for 3 hr at room temperature, then concentrated under reduced pressure to give a residue. The residue was purified by reverse phase flash chromatography to afford 2-{4-azaspiro[2.5]octan-7-yl}-6-{2,8-dimethylimidazo[1,2-b]pyridazin-6-yl}-8-methylisoquinolin-1-one (C35, 155 mg, 64%) as a solid. LCMS (ES, m/z): 414 [M+H]⁺.

### Purification of Compound 398

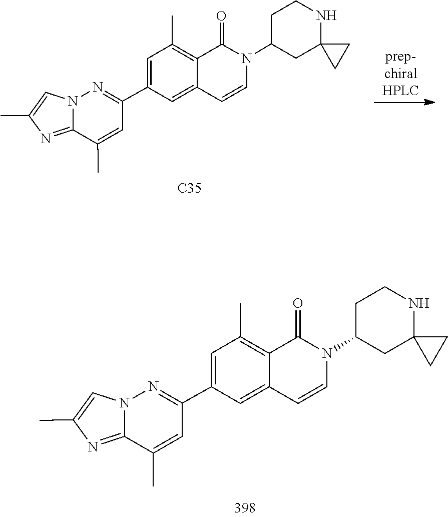 [SMILES: Cc1cn2nc(-c3cc(C)c4c(=O)n(C5CCNC6(CC6)C5)ccc4c3)cc(C)c2n1 ; "C35" ; Cc1cn2nc(-c3cc(C)c4c(=O)n([C@@H]5CCNC6(CC6)C5)ccc4c3)cc(C)c2n1]

C35 was purified by chiral supercritical fluid chromatography (Condition 1, Gradient 1) to afford (R)-6-(2,8-dimethylimidazo[1,2-b]pyridazin-6-yl)-8-methyl-2-(4-azaspiro[2.5]octan-7-yl)isoquinolin-1(2H)-one (Compound 398, 33 mg) as a solid. LCMS (ES, m/z): 414 [M+H]^{+ 1}H NMR (300 MHz, DMSO-d6) δ 8.07 (d, J=3.0 Hz, 2H), 7.84 (s, 1H), 7.69 (s, 1H), 7.48 (d, J=7.5 Hz, 1H), 6.68 (d, J=7.4 Hz, 1H), 5.04 (q, J=10.3, 9.2 Hz, 1H), 3.00 (d, J=12.7 Hz, 1H), 2.90 (s, 3H), 2.70 (dt, J=13.5, 7.5 Hz, 1H), 2.60 (s, 3H), 2.41 (s, 3H), 2.18 (t, J=12.0 Hz, 1H), 2.04 (s, 1H), 1.72 (h, J=4.3, 3.8 Hz, 2H), 1.12 (dd, J=11.9, 3.9 Hz, 1H), 0.63-0.52 (m, 1H), 0.45 (s, 1H), 0.46-0.31 (m, 2H).

### Purification of Compound 397

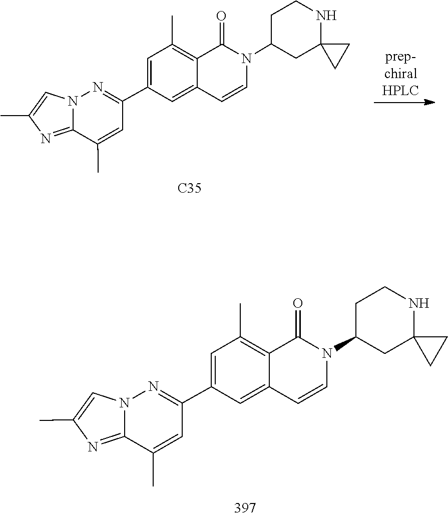 [SMILES: Cc1cn2nc(-c3cc(C)c4c(=O)n(C5CCNC6(CC6)C5)ccc4c3)cc(C)c2n1 ; "C35" ; Cc1cn2nc(-c3cc(C)c4c(=O)n([C@H]5CCNC6(CC6)C5)ccc4c3)cc(C)c2n1]

C35 was further purified by preparative chiral HPLC (Condition 4, Gradient 1) to afford (S)-6-(2,8-dimethylimidazo[1,2-b]pyridazin-6-yl)-8-methyl-2-(4-azaspiro[2.5]octan-7-yl)isoquinolin-1(2H)-one (Compound 397, 34 mg) as a solid. LCMS (ES, m/z): 414 [M+H]^{+ 1}H NMR (300 MHz, DMSO-d6) δ 8.08 (d, J=4.5 Hz, 2H), 7.86 (s, 1H), 7.70 (s, 1H), 7.48 (d, J=7.5 Hz, 1H), 6.70 (d, J=7.4 Hz, 1H), 5.09-4.97 (m, 1H), 3.05 (d, J=12.8 Hz, 1H), 2.90 (s, 3H), 2.76 (d, J=10.3 Hz, 1H), 2.61 (s, 3H), 2.41 (s, 3H), 2.22 (t, J=12.2 Hz, 1H), 1.83-1.70 (m, 2H), 1.30 (s, 0H), 1.25-1.10 (m, 2H), 0.67-0.57 (m, 1H), 0.49 (d, J=9.5 Hz, 2H), 0.42 (d, J=7.4 Hz, 1H).

### Example 102: Synthesis of Compound 390

### Synthesis of Intermediate C36

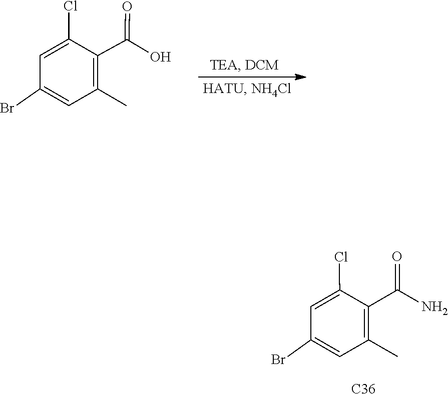

A mixture of 4-bromo-2-chloro-6-methylbenzoic acid (5.0 g, 20.041 mmol, 1.0 equiv), dichloromethane (50 mL), HATU (9.14 g, 24.049 mmol, 1.2 equiv), TEA (8.11 g, 80.164 mmol, 4.0 equiv) and NH₄Cl (3.22 g, 60.123 mmol, 3.0 equiv) was stirred for 6 hr at room temperature. The resulting mixture was diluted with water (50 mL) and extracted with CH₂Cl₂ (3×50 mL). The organic layers were combined, washed with brine (1×100 mL), dried over anhydrous Na₂SO₄, filtered, and the filtrate concentrated under reduced pressure to give a residue. The residue was purified by silica gel column chromatography, eluted with PE/EA (1:3) to afford 4-bromo-2-chloro-6-methylbenzamide (C36, 4.8 g, 96%) as a solid. LCMS (ES, m/z): 248 [M+H]⁺.

### Synthesis of Intermediate C37

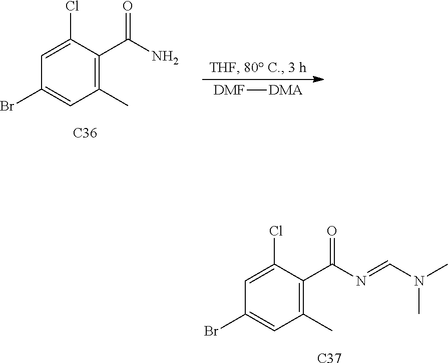

A mixture of 4-bromo-2-chloro-6-methylbenzamide (4.0 g, 16.097 mmol, 1 equiv), THF (20 mL) and DMF-DMA (2.11 g, 17.707 mmol, 1.1 equiv) was stirred for 3 h at 80° C. The resulting mixture was concentrated under reduced pressure to afford 4-bromo-2-chloro-6-methyl-N-[(1E)-(methylamino)methylidene]benzamide (C37, 4.0 g, 85%) as a solid. LCMS (ES, m/z): 303 [M+H]⁺.

### Synthesis of Intermediate C38

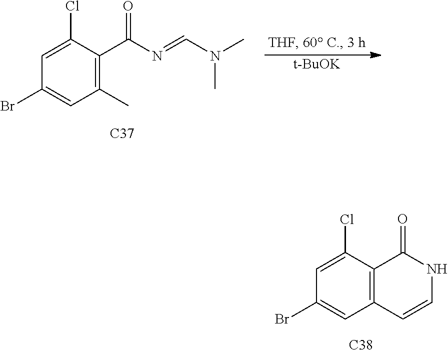 [SMILES: "t-BuOK" ; Cc1cc(Br)cc(Cl)c1C(=O)/N=C/N(C)C ; "C37" ; O=c1[nH]ccc2cc(Br)cc(Cl)c12 ; "C38"]

To a mixture of 4-bromo-2-chloro-N-[(1E)-(dimethylamino)methylidene]-6-methylbenzamide (4.5 g, 14.823 mmol, 1.00 equiv) and THF (10 mL) was added potassium tert-butoxide in THF (22.50 mL, 200.555 mmol, 13.53 equiv) dropwise at 60° C. The resulting mixture was stirred for 2 hr at 60° C. The mixture was concentrated under reduced pressure to give a residue. The residue was acidified to pH 4 with HCl. The resulting mixture was filtered, the filter cake was washed with water and hexane to afford 6-bromo-8-chloro-2H-isoquinolin-1-one (C38, 3.6 g, 93%) as a solid. LCMS (ES, m/z): 258 [M+H]⁺

### Synthesis of Intermediate C39

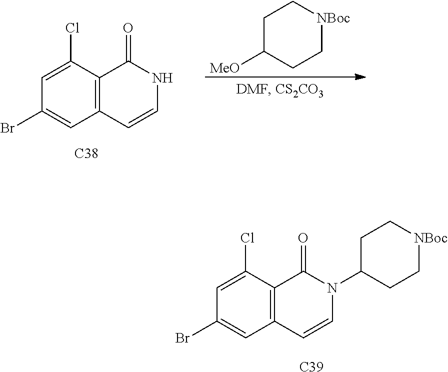 [SMILES: O=c1[nH]ccc2cc(Br)cc(Cl)c12 ; COC1CCN(C(=O)OC(C)(C)C)CC1 ; "C38" ; CC(C)(C)OC(=O)N1CCC(n2ccc3cc(Br)cc(Cl)c3c2=O)CC1 ; "C39"]

A mixture of 6-bromo-8-chloro-2H-isoquinolin-1-one (500 mg, 1.934 mmol, 1.0 equiv), DMF (5 mL), tert-butyl 4-(methanesulfonyloxy)piperidine-1-carboxylate (810.49 mg, 2.901 mmol, 1.5 equiv), and Cs₂CO₃ (1890.64 mg, 5.802 mmol, 3.0 equiv) was stirred overnight at 110° C. The mixture was diluted with water (40 mL) and extracted with ethyl acetate (3×40 mL). The organic layers were combined, washed with water (3×40 mL), dried over anhydrous Na₂SO₄, filtered, and the filtrate concentrated under reduced pressure to give a residue. The residue was purified by reverse phase flash chromatography to afford tert-butyl 4-(6-bromo-8-chloro-1-oxoisoquinolin-2-yl)piperidine-1-carboxylate (C39, 160 mg, 18%) as a solid. LCMS (ES, m/z): 441 [M+H]⁺.

### Synthesis of Intermediate C40

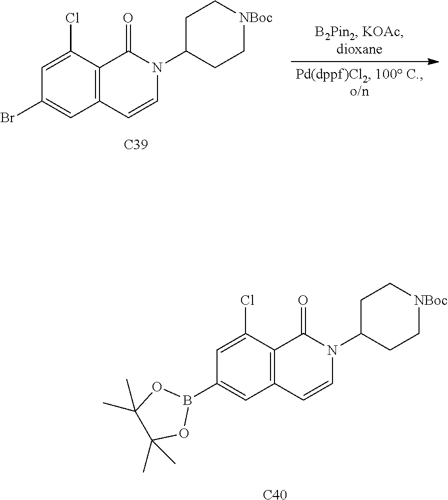 [SMILES: CC(C)(C)OC(=O)N1CCC(n2ccc3cc(Br)cc(Cl)c3c2=O)CC1 ; "C39" ; CC(C)(C)OC(=O)N1CCC(n2ccc3cc(B4OC(C)(C)C(C)(C)O4)cc(Cl)c3c2=O)CC1 ; "C40"]

A mixture of tert-butyl 4-(6-bromo-8-chloro-1-oxoisoquinolin-2-yl)piperidine-1-carboxylate (80 mg, 0.181 mmol, 1.0 equiv), dioxane (1 mL), bis(pinacolato)diboron (55.19 mg, 0.217 mmol, 1.2 equiv), potassium acetate (53.32 mg, 0.543 mmol, 3.0 equiv), and Pd(dppf)Cl₂CH₂Cl₂ (14.75 mg, 0.018 mmol, 0.1 equiv) was stirred overnight at 100° C. under nitrogen atmosphere. LCMS (ES, m/z): 489 [M+H]⁺.

### Synthesis of Intermediate C41

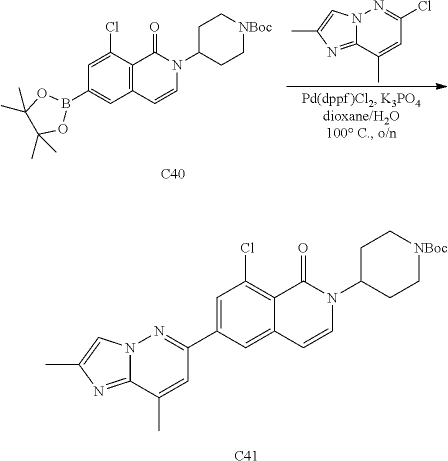 [SMILES: CC(C)(C)OC(=O)N1CCC(n2ccc3cc(B4OC(C)(C)C(C)(C)O4)cc(Cl)c3c2=O)CC1 ; Cc1cn2nc(Cl)cc(C)c2n1 ; "C40" ; Cc1cn2nc(-c3cc(Cl)c4c(=O)n(C5CCN(C(=O)OC(C)(C)C)CC5)ccc4c3)cc(C)c2n1 ; "C41"]

To a mixture of C40 was added water (0.2 mL), 6-chloro-2,8-dimethylimidazo[1,2-b]pyridazine (32.70 mg, 0.180 mmol, 1.0 equiv), K₃PO₄ (38.21 mg, 0.180 mmol, 1.0 equiv), and Pd(dppf)Cl₂CH₂Cl₂ (14.67 mg, 0.018 mmol, 0.1 equiv) at room temperature under a nitrogen atmosphere. The resulting mixture was stirred overnight at 100° C., then diluted with water (5 ml) and extracted with ethyl acetate (3×5 mL). The organic layers were combined, washed with brine (10 mL), dried over anhydrous Na₂SO₄, filtered, and the filtrate concentrated under reduced pressure to give a residue. The residue was purified by silica gel column chromatography, eluted with CH₂Cl₂/MeOH (10:1) to afford tert-butyl 4-(8-chloro-6-{2,8-dimethylimidazo[1,2-b]pyridazin-6-yl}-1-oxoisoquinolin-2-yl)piperidine-1-carboxylate (C41, 70 mg, 76%) as an oil. LCMS (ES, m/z): 508 [M+H]⁺.

### Synthesis of Compound 390

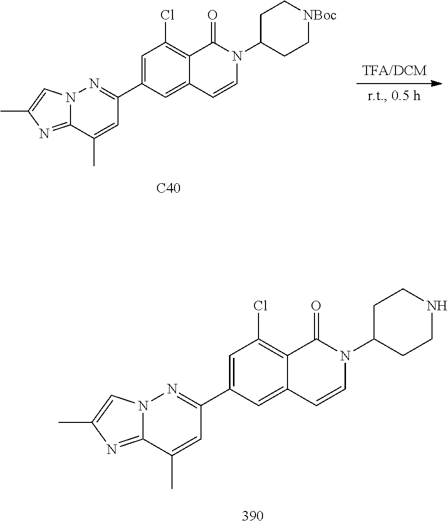 [SMILES: Cc1cn2nc(-c3cc(Cl)c4c(=O)n(C5CCN(C(=O)OC(C)(C)C)CC5)ccc4c3)cc(C)c2n1 ; "C40" ; Cc1cn2nc(-c3cc(Cl)c4c(=O)n(C5CCNCC5)ccc4c3)cc(C)c2n1 ; "390"]

A mixture of tert-butyl 4-(8-chloro-6-{2,8-dimethylimidazo[1,2-b]pyridazin-6-yl}-1-oxoisoquinolin-2-yl)piperidine-1-carboxylate (60 mg, 0.118 mmol, 1 equiv), dichloromethane (1 mL), and trifluoroacetic acid (1 mL, 13.463 mmol, 113.99 equiv) was stirred for 30 min at room temperature, then concentrated under reduced pressure to give a residue. The residue was purified by reverse phase flash chromatography to afford 8-chloro-6-{2,8-dimethylimidazo[1,2-b]pyridazin-6-yl}-2-(piperidin-4-yl)isoquinolin-1-one; trifluoroacetaldehyde (Compound 390, 12 mg, 20%) as a solid. LCMS (ES, m/z): 408 [M+H]^{+ 1}H NMR (300 MHz, DMSO-d6) δ 8.84 (d, J=11.1 Hz, 1H), 8.59 (d, J=11.7 Hz, 1H), 8.33 (d, J=1.8 Hz, 1H), 8.23 (s, 1H), 8.15 (d, J=1.7 Hz, 1H), 7.95 (s, 1H), 7.49 (d, J=7.6 Hz, 1H), 6.83 (d, J=7.5 Hz, 1H), 5.03 (ddt, J=11.9, 8.2, 4.4 Hz, 1H), 3.48 (d, J=12.2 Hz, 2H), 3.16 (q, J=11.7 Hz, 2H), 2.65 (s, 3H), 2.46 (s, 3H), 2.14 (qd, J=12.6, 3.6 Hz, 2H), 2.00 (d, J=12.3 Hz, 2H).

### Example 103: Synthesis of Compound 391

### Synthesis of Intermediate C41

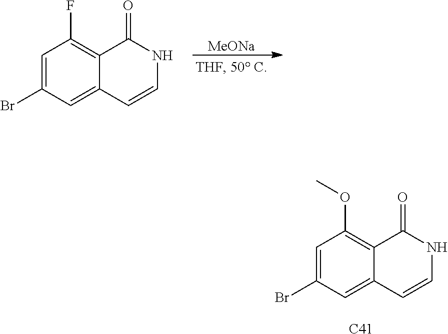

To a stirred mixture of 6-bromo-8-fluoro-2H-isoquinolin-1-one (1 g, 4.13 mmol, 1 equiv) and MeONa (260 mg, 1.96 mmol, 1.2 equiv) was added THF (10 mL) at room temperature. The resulting mixture was stirred for 16 hr at 50° C. The resulting mixture was concentrated under vacuum, then quenched with water (20 mL) and extracted with ethyl acetate (3×20 mL). The organic layers were combined, washed with brine (2×20 mL), dried over anhydrous Na₂SO₄, and filtered. After filtration, the filtrate was concentrated under reduced pressure to afford 6-bromo-8-methoxyisoquinolin-1(2H)-one (C41, 840 mg, 80%) as a solid LCMS (ES, m/z): 254 [M+H]⁺.

### Synthesis of Intermediate C42

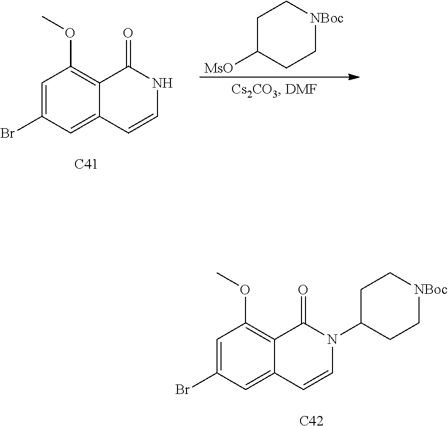 [SMILES: COc1cc(Br)cc2cc[nH]c(=O)c12 ; CC(C)(C)OC(=O)N1CCC(OS(C)(=O)=O)CC1 ; "C41" ; COc1cc(Br)cc2ccn(C3CCN(C(=O)OC(C)(C)C)CC3)c(=O)c12 ; "C42"]

To a stirred solution of 6-bromo-8-methoxy-2H-isoquinolin-1-one (200 mg, 0.79 mmol, 1 equiv) in dimethylacetamide (2 mL) was added tert-butyl 4-(methanesulfonyloxy)piperidine-1-carboxylate (430 mg, 1.58 mmol, 2 equiv) and Cs₂CO₃ (760 mg, 2.37 mmol, 3 equiv) in portions at room temperature. The mixture was stirred for 4 hr at 120° C. The reaction mixture was quenched with water (10 mL). The resulting mixture was extracted with ethyl acetate (3×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with PE:EA (2:1) to afford tert-butyl 4-(6-bromo-8-methoxy-1-oxoisoquinolin-2-yl)piperidine-1-carboxylate (C42, 150 mg, 43%) as a solid. LCMS (ES, m/z): 439 [M+H]⁺.

### Synthesis of Intermediate C43

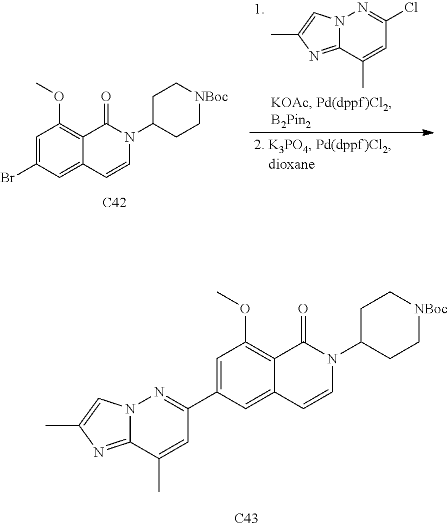 [SMILES: COc1cc(Br)cc2ccn(C3CCN(C(=O)OC(C)(C)C)CC3)c(=O)c12 ; Cc1cn2nc(Cl)cc(C)c2n1 ; "C42" ; COc1cc(-c2cc(C)c3nc(C)cn3n2)cc2ccn(C3CCN(C(=O)OC(C)(C)C)CC3)c(=O)c12 ; "C43"]

To a stirred solution of 6-chloro-2,8-dimethylimidazo[1,2-b]pyridazine (124 mg, 0.686 mmol, 1.5 equiv) and B₂Pin₂ (230 mg, 0.914 mmol, 2.0 equiv) in dioxane (6 mL) was added potassium acetate (112 mg, 1.143 mmol2.5 equiv) and Pd(dppf)Cl₂ (38 mg, 0.0457 mmol, 0.1 equiv) at room temperature under a nitrogen atmosphere. The reaction mixture was stirred at 100° C. overnight. The resulting mixture was cooled to room temperature. To the above mixture were added tert-butyl 4-(6-bromo-8-methoxy-1-oxoisoquinolin-2-yl)piperidine-1-carboxylate (200 mg, 0.457 mmol, 1.0 equiv), K₃PO₄ (290 mg, 1.371 mmol, 3.0 equiv), H₂O (1 mL) and Pd(dppf)Cl₂ (38 mg, 0.0457 mmol, 0.1 equiv) at room temperature under a nitrogen atmosphere. The reaction mixture was stirred for additional 4 hr at 100° C. The reaction was quenched with water (50 mL) at room temperature. The resulting mixture was extracted with ethyl acetate (2×50 mL). The organic layers were combined, washed with brine (2×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(6-{2,8-dimethylimidazo[1,2-b]pyridazin-6-yl}-8-methoxy-1-oxoisoquinolin-2-yl)piperidine-1-carboxylate (C43, 150 mg, 65%) as a solid. LCMS (ES, m/z): 504 [M+H]⁺.

### Synthesis of Compound 391

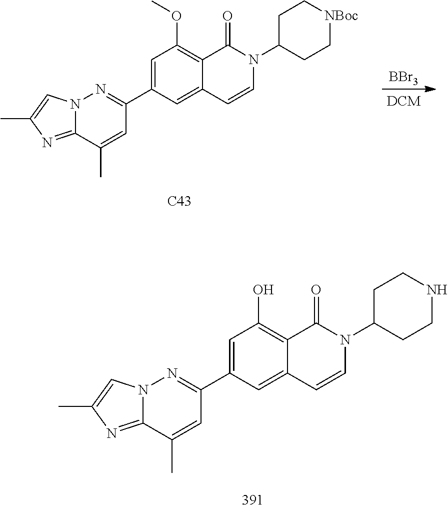

A solution of tert-butyl 4-(6-{2,8-dimethylimidazo[1,2-b]pyridazin-6-yl}-8-methoxy-1-oxoisoquinolin-2-yl)piperidine-1-carboxylate (50 mg, 0.099 mmol, 1 equiv) and BBr₃ (124 mg, 0.495 mmol, 5.0 equiv) in dichloromethane (30 mL) was stirred for 1 hr at 25° C. The resulting mixture was concentrated in vacuum to give a residue. The residue was purified by Prep-HPLC (Condition 4, Gradient 1) to afford 6-(2,8-dimethylimidazo[1,2-b]pyridazin-6-yl)-8-hydroxy-2-(piperidin-4-yl)isoquinolin-1(2H)-one (Compound 391, 17 mg, 40%) as a solid. LCMS (ES, m/z): 390 [M+H]^{+ 1}H NMR (300 MHz, DMSO-d₆) δ 8.40 (d, J=1.1 Hz, 1H), 8.21 (s, 1H), 7.87 (d, J=1.7 Hz, 1H), 7.58-7.44 (m, 2H), 6.97 (d, J=7.6 Hz, 1H), 5.13-4.98 (m, 1H), 3.23-3.09 (m, 2H), 2.70 (d, J=1.0 Hz, 3H), 2.54 (d, J=1.0 Hz, 3H), 2.27-2.13 (m, 2H), 2.04 (d, J=12.9 Hz, 2H).

### Example 104: Synthesis of Compounds 382 and 392

### Synthesis of Intermediate C44

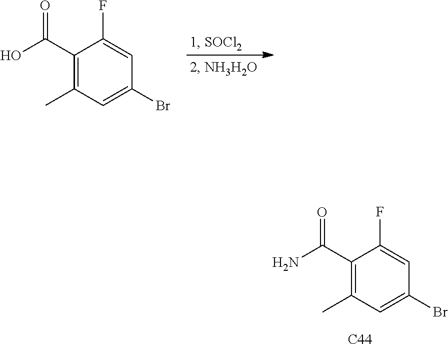

A solution of 4-bromo-2-fluoro-6-methylbenzoic acid (25 g, 107.28 mmol, 1 equiv) and SOCl₂ (200 mL) was stirred for 16 h at 85° C. The resulting mixture was concentrated under reduced pressure to give a residue. The residue was diluted with THF (100 mL), then added dropwise to NH₃H₂O (200 mL) at 0° C. The resulting mixture was stirred for an additional 2 hr at room temperature. The resulting mixture was extracted with ethyl acetate (3×100 mL). The organic layers were combined, washed with brine (2×50 mL), and dried over anhydrous Na₂SO₄, and filtered. After filtration, the filtrate was concentrated under reduced pressure to afford 4-bromo-2-fluoro-6-methylbenzamide (C44, 24 g, 96%) as a solid. LCMS (ES, m/z): 232 [M+H]⁺.

### Synthesis of Intermediate C45

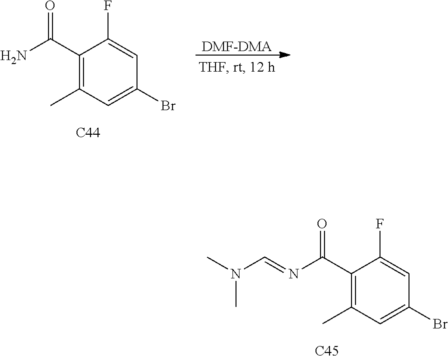

To a stirred solution of 4-bromo-2-fluoro-6-methylbenzamide (24 g, 103.425 mmol, 1.00 equiv) in THF (300 mL) was added DMF-DMA (13.56 g, 113.768 mmol, 1.1 equiv) at room temperature. The reaction mixture was stirred for 12 hr at room temperature. The resulting mixture was concentrated under reduced pressure to afford 4-bromo-N-[(1E)-(dimethylamino)methylidene]-2-fluoro-6-methylbenzamide (C45, 22.2 g, 74%) as a solid. LCMS (ES, m/z): 287 [M+H]⁺.

### Synthesis of Intermediate C46

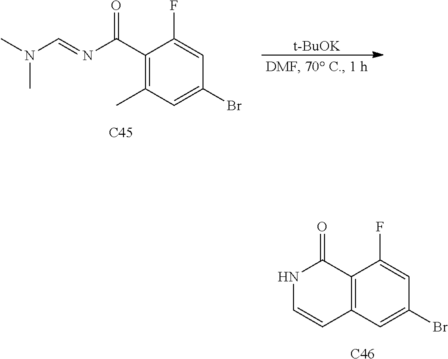 [SMILES: "t-BuOK" ; Cc1cc(Br)cc(F)c1C(=O)/N=C/N(C)C ; "C45" ; O=c1[nH]ccc2cc(Br)cc(F)c12 ; "C46"]

To a stirred mixture of 4-bromo-N-[(1E)-(dimethylamino)methylidene]-2-fluoro-6-methylbenzamide (20 g, 69.654 mmol, 1 equiv) in THE (300 mL) was added t-BuOK (9.38 g, 83.585 mmol, 1.2 equiv) in portions at room temperature under a nitrogen atmosphere. The reaction mixture was stirred for 1 hr at 70° C. The resulting mixture was concentrated under reduced pressure. The residue was acidified to pH 5 with 1 M HCl. A precipitate formed that was collected by filtration and purified by reverse phase chromatography (Condition 2, Gradient 13)to afford 6-bromo-8-fluoro-2H-isoquinolin-1-one (C46, 11.6 g, 69%) as a solid. LCMS (ES, m/z): 242 [M+H]⁺.

### Synthesis of Intermediate C47

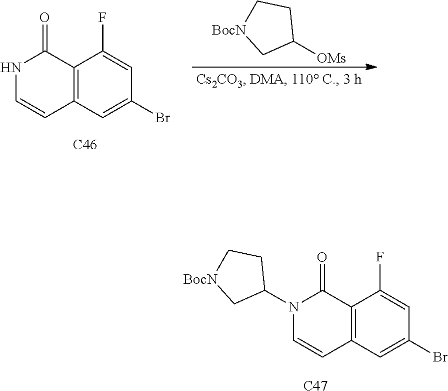 [SMILES: O=c1[nH]ccc2cc(Br)cc(F)c12 ; CC(C)(C)OC(=O)N1CCC(OS(C)(=O)=O)C1 ; "C46" ; CC(C)(C)OC(=O)N1CCC(n2ccc3cc(Br)cc(F)c3c2=O)C1 ; "C47"]

To a stirred solution of 6-bromo-8-fluoro-2H-isoquinolin-1-one (1 g, 4.131 mmol, 1 equiv) and tert-butyl 3-(methanesulfonyloxy)pyrrolidine-1-carboxylate (1.64 g, 6.197 mmol, 1.5 equiv) in DMA (15 mL) was added Cs₂CO₃ (4 g, 12.393 mmol, 3 equiv) in portions at room temperature. The resulting mixture was stirred for 5 hr at 110° C. The mixture was allowed to cool down to room temperature. The residue was purified by reverse flash chromatography to afford tert-butyl 3-(6-bromo-8-fluoro-1-oxoisoquinolin-2-yl)pyrrolidine-1-carboxylate (C47, 0.68 g, 40%) as a solid. LCMS (ES, m/z): 411 [M+H]⁺

### Synthesis of Intermediate C48

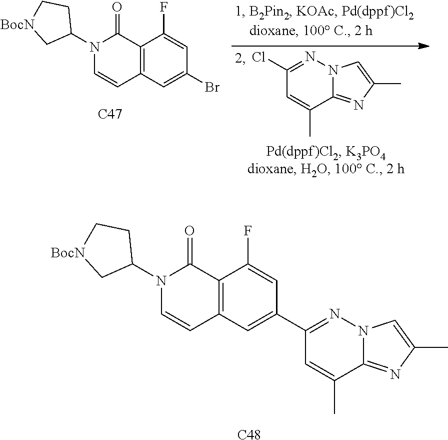 [SMILES: "C47" ; CC(C)(C)OC(=O)N1CCC(n2ccc3cc(Br)cc(F)c3c2=O)C1 ; Cc1cn2nc(Cl)cc(C)c2n1 ; Cc1cn2nc(-c3cc(F)c4c(=O)n(C5CCN(C(=O)OC(C)(C)C)C5)ccc4c3)cc(C)c2n1 ; "C48"]

To a stirred solution of tert-butyl 3-(6-bromo-8-fluoro-1-oxoisoquinolin-2-yl)pyrrolidine-1-carboxylate (180 mg, 0.438 mmol, 1 equiv) and bis(pinacolato)diboron (133 mg, 0.526 mmol, 1.2 equiv) in dioxane (10 mL) was added potassium acetate (86 mg, 0.876 mmol, 2 equiv) and Pd(dppf)Cl₂ (32 mg, 0.044 mmol, 0.1 equiv) in portions at room temperature under a nitrogen atmosphere. The resulting mixture was stirred for 2 hr at 100° C. under a nitrogen atmosphere. The mixture was cooled to room temperature. To the reaction mixture was added 6-chloro-2,8-dimethylimidazo[1,2-b]pyridazine (80 mg, 0.438 mmol, 1 equiv), K₃PO₄ (186 mg, 0.876 mmol, 2 equiv), H₂O (2 mL) and Pd(dppf)Cl₂ (32 mg, 0.044 mmol, 0.1 equiv) in portions at room temperature. The resulting mixture was stirred for an additional 2 hr at 100° C., then cooled to room temperature. The reaction was quenched with water (10 mL) and extracted with ethyl acetate (3×3 mL). The organic layers were combined, washed with brine (1×10 mL), dried over anhydrous Na₂SO₄, and filtered. After filtration, the filtrate was concentrated under reduced pressure to give a residue. The residue was purified by reverse flash chromatography to afford tert-butyl 3-(6-{2,8-dimethylimidazo[1,2-b]pyridazin-6-yl}-8-fluoro-1-oxoisoquinolin-2-yl)pyrrolidine-1-carboxylate (91 mg, 434%) as a solid. LCMS (ES, m/z): 478 [M+H]⁺.

### Synthesis of Compounds 382 and 392

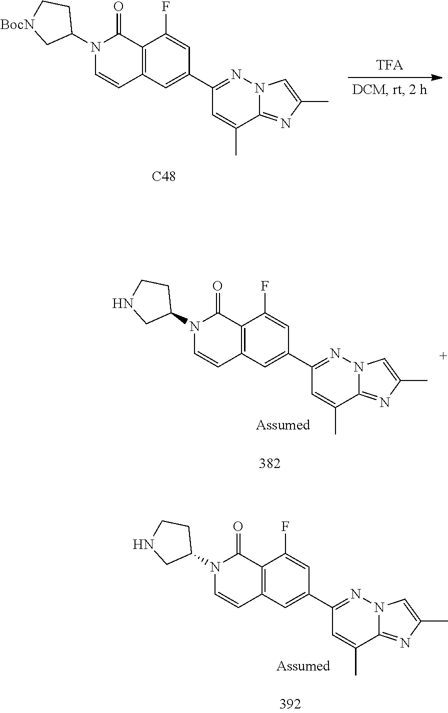 [SMILES: Cc1cn2nc(-c3cc(F)c4c(=O)n(C5CCN(C(=O)OC(C)(C)C)C5)ccc4c3)cc(C)c2n1 ; "C48" ; Cc1cn2nc(-c3cc(F)c4c(=O)n([C@@H]5CCNC5)ccc4c3)cc(C)c2n1 ; Cc1cn2nc(-c3cc(F)c4c(=O)n([C@H]5CCNC5)ccc4c3)cc(C)c2n1]

A solution of tert-butyl 3-(6-{2,8-dimethylimidazo[1,2-b]pyridazin-6-yl}-8-fluoro-1-oxoisoquinolin-2-yl)pyrrolidine-1-carboxylate (100 mg, 0.209 mmol, 1 equiv) in TFA (1 mL) and DCM (1 mL) was stirred for 2 hr at room temperature. The resulting mixture was concentrated under vacuum to give a residue. The residue was basified to pH 10 with NH₃ (g) in methanol. The resulting mixture was concentrated under reduced pressure to give a residue. The residue was purified by reverse flash chromatography to afford 6-{2,8-dimethylimidazo[1,2-b]pyridazin-6-yl}-8-fluoro-2-(pyrrolidin-3-yl)isoquinolin-1-one (60 mg, 75%) as a solid that was separated by Preparative Chiral-HPLC (Condition 5, Gradient 1) to afford (R)-6-(2,8-dimethylimidazo[1,2-b]pyridazin-6-yl)-8-fluoro-2-(pyrrolidin-3-yl)isoquinolin-1(2H)-one (Compound 382, 16.1 mg, 20%) as a solid and (S)-6-(2,8-dimethylimidazo[1,2-b]pyridazin-6-yl)-8-fluoro-2-(pyrrolidin-3-yl)isoquinolin-1(2H)-one (Compound 392, 12.1 mg, 15%) as a solid.

| Compound No. | Analysis Data |
|---|---|
| 382 | LCMS (ES, m/z): 378 [M + H] ⁺ |
| | ¹H NMR (300 MHz, DMSO-d₆) 8 8.17 (s, 1H), 8.10 |
| | (s, 1H), 7.88-7.76 (m, 2H), 7.68 (d, J = 7.7 Hz, 1H), |
| | 6.78 (d, J = 7.5 Hz, 1H), 5.33 (s, 1H), 3.10 (s, 2H), |
| | 2.85 (d, J = 9.7 Hz, 2H), 2.62 (s, 3H), 2.42 (s, 3H), |
| | 2.23 (s, 1H), 1.75 (s, 1H). |
| 392 | LCMS (ES, m/z): 378 [M + H] ⁺ |
| | ¹H NMR (300 MHZ, DMSO-d₆) 8 8.17 (s, 1H), 8.10 |
| | (s, 1H), 7.88-7.76 (m, 2H), 7.68 (d, J = 7.7 Hz, 1H), |
| | 6.78 (d, J = 7.5 Hz, 1H), 5.33 (s, 1H), 3.10 (s, 2H), |
| | 2.85 (d, J = 9.7 Hz, 2H), 2.62 (s, 3H), 2.42 (s, 3H), |
| | 2.23 (s, 1H), 1.75 (s, 1H). |

### Example 105: Synthesis of Compounds 410 and 411

### Synthesis of Intermediate C49

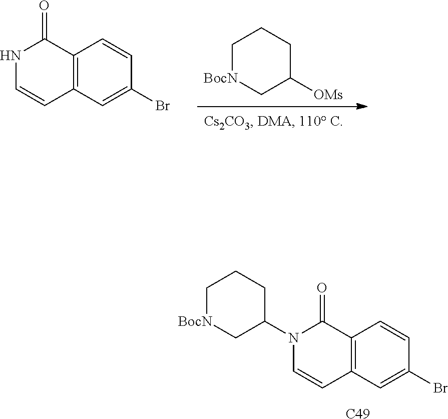 [SMILES: O=c1[nH]ccc2cc(Br)ccc12 ; CC(C)(C)OC(=O)N1CCCC(OS(C)(=O)=O)C1 ; CC(C)(C)OC(=O)N1CCCC(n2ccc3cc(Br)ccc3c2=O)C1 ; "C49"]

To a stirred solution of 6-bromo-2H-isoquinolin-1-one (1 g, 4.463 mmol, 1 equiv) and tert-butyl 3-(methanesulfonyloxy)piperidine-1-carboxylate (2.49 g, 8.926 mmol, 2.0 equiv) in DMA (20 mL) was added Cs₂CO₃ (4.36 g, 13.389 mmol, 3.0 equiv) in portions at room temperature. The resulting mixture was stirred for 12 hr at 110° C., then cooled to room temperature. The residue was purified by reverse flash chromatography to afford tert-butyl 3-(6-bromo-1-oxoisoquinolin-2-yl)piperidine-1-carboxylate (C49, 200 mg, 11%) as a solid. LCMS (ES, m/z): 407 [M+H]⁺.

### Synthesis of Intermediate C50

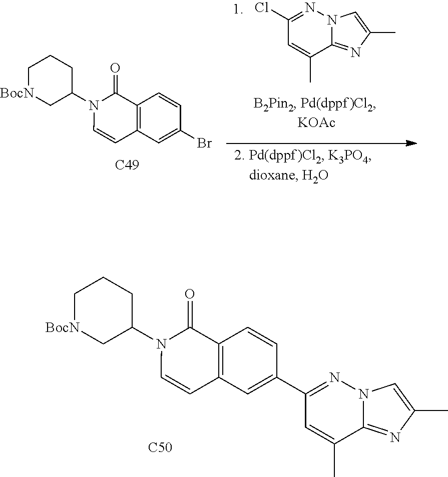 [SMILES: CC(C)(C)OC(=O)N1CCCC(n2ccc3cc(Br)ccc3c2=O)C1 ; Cc1cn2nc(Cl)cc(C)c2n1 ; "C49" ; "C50" ; Cc1cn2nc(-c3ccc4c(=O)n(C5CCCN(C(=O)OC(C)(C)C)C5)ccc4c3)cc(C)c2n1]

To a solution of 6-chloro-2,8-dimethylimidazo[1,2-b]pyridazine (100 mg, 0.551 mmol, 1 equiv) and 4,4,5,5-tetramethyl-2-(tetramethyl-1,3,2-dioxaborolan-2-yl)-1,3,2-dioxaborolane (168 mg, 0.661 mmol, 1.2 equiv) in dioxane (5 mL) was added potassium acetate (162 mg, 1.653 mmol, 3.0 equiv) and Pd(dppf)Cl₂·CH₂Cl₂ (45 mg, 0.055 mmol, 0.1 equiv). After stirring for 12 hr at 80° C. under a nitrogen atmosphere, then cooled to room temperature. To the reaction mixture was added tert-butyl 3-(6-bromo-1-oxoisoquinolin-2-yl)piperidine-1-carboxylate (247 mg, 0.606 mmol, 1.1 equiv), K₃PO₄ (350 mg, 1.653 mmol, 3.0 equiv), Pd(dppf)Cl₂·CH₂Cl₂ (46 mg, 0.055 mmol, 0.1 equiv) and H₂O (1 mL) at room temperature under a nitrogen atmosphere. The reaction mixture was stirred for an additional 1 hr at 100° C. under a nitrogen atmosphere, then concentrated under reduced pressure to give a residue. The residue was purified by to yield tert-butyl 3-(6-{2,8-dimethylimidazo[1,2-b]pyridazin-6-yl}-1-oxoisoquinolin-2-yl)piperidine-1-carboxylate (C50, 100 mg, 38%) as a solid. LCMS (ES, m/z): 474 [M+H]⁺.

### Synthesis of Compounds 410 and 411

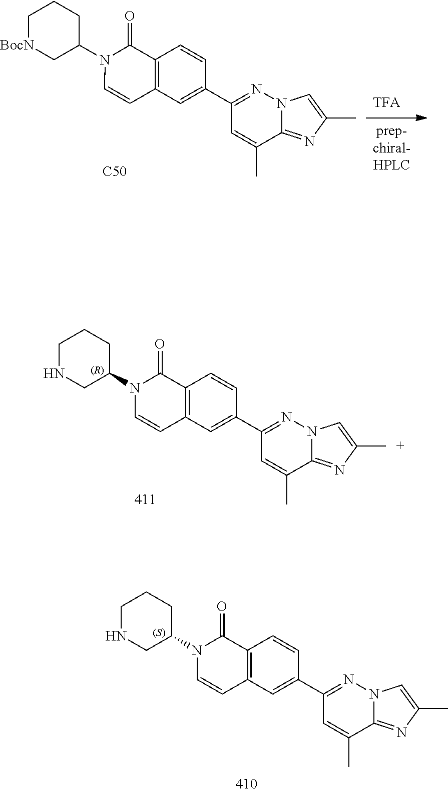

A solution of tert-butyl 3-(6-{2,8-dimethylimidazo[1,2-b]pyridazin-6-yl}-1-oxoisoquinolin-2-yl)piperidine-1-carboxylate (100 mg, 0.211 mmol, 1 equiv) and TFA (0.5 mL) in dichloromethane (2 mL) was stirred for 1 hr at room temperature. The reaction mixture was basified to pH 8 with NH₃ in methanol, then concentrated under reduced pressure to give a residue. The residue was purified by reverse flash chromatography to yield 6-(2,8-dimethylimidazo[1,2-b]pyridazin-6-yl)-2-(piperidin-3-yl)isoquinolin-1(2H)-one (50 mg, 63%) as a solid that was separated by Preparative Chiral-HPLC (Condition 6, Gradient 1) to afford 6-{2,8-dimethylimidazo[1,2-b]pyridazin-6-yl}-2-[(3R)-piperidin-3-yl]isoquinolin-1-one (Compound 411, 13 mg, 16%) as a solid and 6-{2,8-dimethylimidazo[1,2-b]pyridazin-6-yl}-2-[(3S)-piperidin-3-yl]isoquinolin-1-one (Compound 410, 18 mg, 22%) as a solid.

| Compound | |
|---|---|
| No. | Analysis Data |
| 410 | LCMS (ES, m/z): 374 [M + H]⁺ |
| | ¹H NMR (300 MHz, DMSO-d₆) δ 8.41-8.31 (m, 2H), 8.20-8.08 (m, 2H), |
| | 7.77 (s, 1H), 7.67 (d, J = 7.5 Hz, 1H), 6.78 (d, J = 7.5 Hz, 1H), 4.86 (s, |
| | 1H), 2.94 (t, J = 13.2 Hz, 2H), 2.78 (d, J = 10.9 Hz, 1H), 2.64 (d, J = 1.1 |
| | Hz, 3H), 2.43 (d, J = 0.8 Hz, 4H), 1.86 (d, J = 9.4 Hz, 2H), 1.77 (d, J = |
| | 12.4 Hz, 1H), 1.59 (s, 1H). |
| 411 | LCMS (ES, m/z): 374 [M + H]⁺ |
| | ¹H NMR (300 MHz, DMSO-d₆) δ 8.41-8.31 (m, 2H), 8.20-8.08 (m, 2H), |
| | 7.77 (d, J = 1.2 Hz, 1H), 7.67 (d, J = 7.5 Hz, 1H), 6.77 (d, J = 7.6 Hz, |
| | 1H), 4.85 (s, 1H), 2.93 (t, J = 15.3 Hz, 2H), 2.74 (t, J = 11.1 Hz, 1H), 2.64 |
| | (d, J = 1.0 Hz, 3H), 2.43 (d, J = 0.8 Hz, 4H), 1.85 (d, J = 8.5 Hz, 2H), |
| | 1.76 (d, J = 12.6 Hz, 1H), 1.57 (s, 1H). |

### Example 106: Synthesis of Compound 393

### Synthesis of Intermediate C51

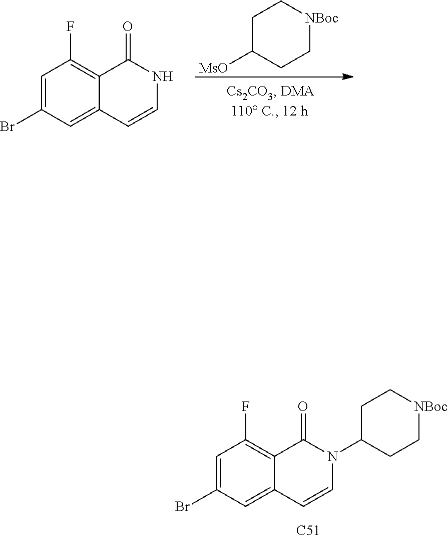 [SMILES: O=c1[nH]ccc2cc(Br)cc(F)c12 ; CC(C)(C)OC(=O)N1CCC(OS(C)(=O)=O)CC1 ; CC(C)(C)OC(=O)N1CCC(n2ccc3cc(Br)cc(F)c3c2=O)CC1 ; "C51"]

To a stirred solution of 6-bromo-8-fluoro-2H-isoquinolin-1-one (1 g, 4.131 mmol, 1 equiv) and tert-butyl 4-(methanesulfonyloxy)piperidine-1-carboxylate (1.7 g, 6.197 mmol, 1.5 equiv) in DMA (20 mL) was added Cs₂CO₃ (2.68 g, 8.262 mmol, 2.0 equiv) in portions at room temperature. The resulting mixture was stirred for 12 hr at 110° C., then cooled to room temperature. The residue was purified by reverse flash chromatography to afford tert-butyl 4-(6-bromo-8-fluoro-1-oxoisoquinolin-2-yl)piperidine-1-carboxylate (C51 380 mg, 21%) as a solid. LCMS (ES, m/z): 425 [M+H]⁺.

### Synthesis of Intermediate C52

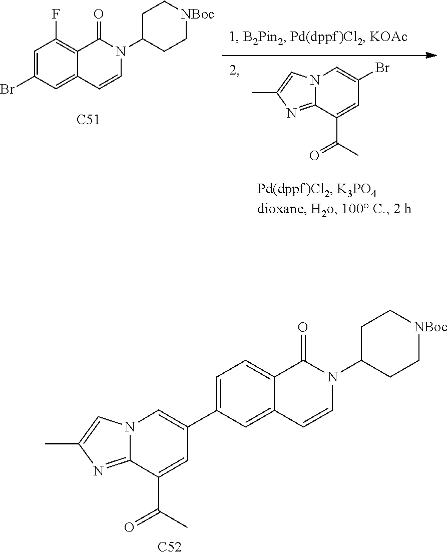 [SMILES: "C51" ; CC(C)(C)OC(=O)N1CCC(n2ccc3cc(Br)cc(F)c3c2=O)CC1 ; CC(=O)c1cc(Br)cn2cc(C)nc12 ; CC(=O)c1cc(-c2ccc3c(=O)n(C4CCN(C(=O)OC(C)(C)C)CC4)ccc3c2)cn2cc(C)nc12 ; "C52"]

To a stirred solution of tert-butyl 4-(6-bromo-8-fluoro-1-oxoisoquinolin-2-yl)piperidine-1-carboxylate (50 mg, 0.118 mmol, 1 equiv) and bis(pinacolato)diboron (36 mg, 0.142 mmol, 1.2 equiv) in dioxane (5 mL) was added KOAc (23 mg, 0.236 mmol, 2 equiv) and Pd(dppf)Cl₂ (9 mg, 0.012 mmol, 0.1 equiv) in portions at room temperature under a nitrogen atmosphere. The resulting mixture was stirred for 2 hr at 100° C. under a nitrogen atmosphere, then cooled to room temperature. To the reaction mixture was added 1-{6-bromo-2-methylimidazo[1,2-a]pyridin-8-yl}ethanone (30 mg, 0.118 mmol, 1 equiv), K₃PO₄ (50 mg, 0.236 mmol, 2 equiv), H₂O (1 mL), and Pd(dppf)Cl₂ (9 mg, 0.012 mmol, 0.1 equiv) in portions at room temperature. The resulting mixture was stirred for an additional 2 h at 100° C., then cooled to room temperature. The reaction mixture was quenched with water (20 mL) and extracted with ethyl acetate (3×20 mL). The organic layers were combined, washed with brine (1×10 mL), dried over anhydrous Na₂SO₄, and filtered. After filtration, the filtrate was concentrated under reduced pressure to give a residue. The residue was purified by reverse flash chromatography to afford tert-butyl 4-(6-{8-acetyl-2-methylimidazo[1,2-a]pyridin-6-yl}-8-fluoro-1-oxoisoquinolin-2-yl)piperidine-1-carboxylate (C52, 33 mg, 54%) as a solid. LCMS (ES, m/z): 519 [M+H]⁺

### Synthesis of Compound 393

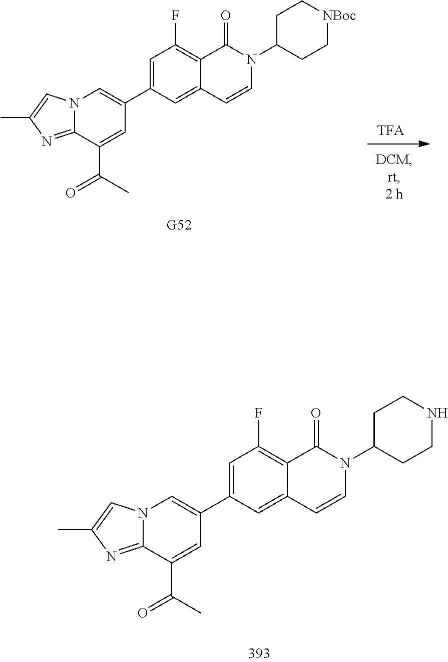 [SMILES: CC(=O)c1cc(-c2cc(F)c3c(=O)n(C4CCN(C(=O)OC(C)(C)C)CC4)ccc3c2)cn2cc(C)nc12 ; CC(=O)c1cc(-c2cc(F)c3c(=O)n(C4CCNCC4)ccc3c2)cn2cc(C)nc12]

A solution of tert-butyl 4-(6-{8-acetyl-2-methylimidazo[1,2-a]pyridin-6-yl}-8-fluoro-1-oxoisoquinolin-2-yl)piperidine-1-carboxylate (32 mg, 0.062 mmol, 1 equiv) in trifluoroacetic acid (1 mL) and dichloromethane (1 mL) was stirred for 2 hr at room temperature. The resulting mixture was concentrated under vacuum to give a residue. The residue was basified to pH 8 with 7 M NH₃ (g) in methanol. The resulting mixture was concentrated under vacuum to give a residue. The residue was purified by reverse flash chromatography to afford 6-{8-acetyl-2-methylimidazo[1,2-a]pyridin-6-yl}-8-fluoro-2-(piperidin-4-yl)isoquinolin-1-one 2,2,2-trifluoroacetate (Compound 393, 16 mg, 61%) as a solid. LCMS (ES, m/z): 419 [M+H]¹H NMR (300 MHz, DMSO-d₆) δ 9.64 (s, 1H), 8.86 (s, 2H), 8.63 (d, J=11.5 Hz, 1H), 8.16-8.05 (m, 2H), 7.88 (d, J=12.8 Hz, 1H), 7.50 (d, J=7.6 Hz, 1H), 6.81 (dd, J=7.6, 2.1 Hz, 1H), 5.06 (s, 1H), 3.48 (d, J=12.4 Hz, 2H), 3.18 (d, J=12.5 Hz, 2H), 2.94 (s, 3H), 2.55 (s, 3H), 2.23-2.05 (m, 2H), 2.05-1.94 (m, 2H).

### Example 107: Synthesis of Compound 383

### Synthesis of Intermediate C53

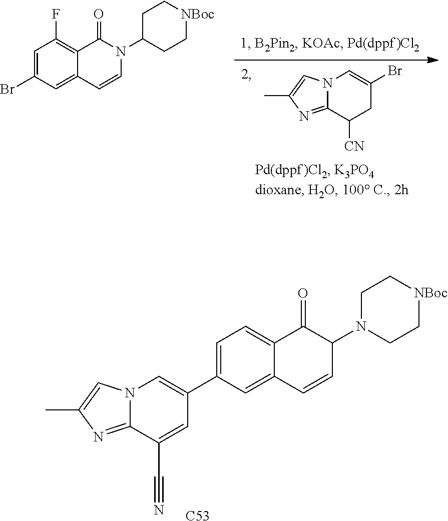 [SMILES: CC(C)(C)OC(=O)N1CCC(n2ccc3cc(Br)cc(F)c3c2=O)CC1 ; Cc1cn2c(n1)C(C#N)CC(Br)=C2 ; Cc1cn2cc(-c3ccc4c(c3)C=CC(N3CCN(C(=O)OC(C)(C)C)CC3)C4=O)cc(C#N)c2n1 ; "C53"]

To a stirred solution of tert-butyl 4-(6-bromo-8-fluoro-1-oxoisoquinolin-2-yl)piperidine-1-carboxylate (45 mg, 0.106 mmol, 1 equiv) and bis(pinacolato)diboron (32 mg, 0.127 mmol, 1.2 equiv) in dioxane (5 mL) was added KOAc (21 mg, 0.212 mmol, 2 equiv) and Pd(dppf)Cl₂ (8 mg, 0.011 mmol, 0.1 equiv) in portions at room temperature under a nitrogen atmosphere. The resulting mixture was stirred for 2 hr at 100° C. under nitrogen atmosphere, then cooled to room temperature. To the reaction mixture was added 6-bromo-2-methylimidazo[1,2-a]pyridine-8-carbonitrile (25 mg, 0.106 mmol, 1 equiv), K₃PO₄ (44.92 mg, 0.212 mmol, 2 equiv), H₂O (1 mL), and Pd(dppf)Cl₂ (7.74 mg, 0.011 mmol, 0.1 equiv) in portions at room temperature. The resulting mixture was stirred for an additional 2 hr at 100° C., then cooled to room temperature, quenched with water (10 mL) and extracted with ethyl acetate (3×20 mL). The organic layers were combined, washed with brine (1×20 mL), dried over anhydrous Na₂SO₄, and filtered. After filtration, the filtrate was concentrated under reduced pressure to give a residue. The residue was purified by reverse flash chromatography to afford tert-butyl 4-(6-{8-cyano-2-methylimidazo[1,2-a]pyridin-6-yl}-8-fluoro-1-oxoisoquinolin-2-yl)piperidine-1-carboxylate (C53, 37 mg, 69%) as a solid. LCMS (ES, m/z): 502 [M+H]⁺.

### Synthesis of Compound 383

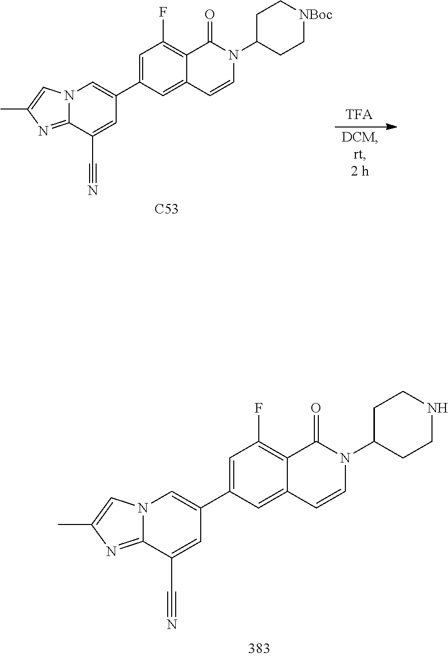 [SMILES: Cc1cn2cc(-c3cc(F)c4c(=O)n(C5CCN(C(=O)OC(C)(C)C)CC5)ccc4c3)cc(C#N)c2n1 ; "C53" ; Cc1cn2cc(-c3cc(F)c4c(=O)n(C5CCNCC5)ccc4c3)cc(C#N)c2n1]

A solution of tert-butyl 4-(6-{8-cyano-2-methylimidazo[1,2-a]pyridin-6-yl}-8-fluoro-1-oxoisoquinolin-2-yl)piperidine-1-carboxylate (35 mg, 0.070 mmol, 1 equiv) in trifluoroacetic acid (2 mL) and dichloromethane (2 mL) was stirred for 2 hr at room temperature. The resulting mixture was concentrated under reduced pressure to give a residue. The residue was basified to pH 9 with 7 M NH₃ (g) in methanol. The resulting mixture was concentrated under reduced pressure to give a residue. The residue was purified by reverse flash chromatography to afford 6-[8-fluoro-1-oxo-2-(piperidin-4-yl)isoquinolin-6-yl]-2-methylimidazo[1,2-a]pyridine-8-carbonitrile (Compound 383, 20 mg, 50%) as a solid. LCMS (ES, m/z): 402 [M+H]^{+ 1}H NMR (300 MHz, DMSO-d₆) δ 9.39 (d, J=1.8 Hz, 1H), 8.45 (d, J=1.8 Hz, 1H), 7.95-7.86 (m, 2H), 7.68 (dd, J=13.3, 1.8 Hz, 1H), 7.60 (d, J=7.6 Hz, 1H), 6.66 (dd, J=7.5, 2.1 Hz, 1H), 4.82 (dd, J=11.2, 6.3 Hz, 1H), 3.08 (d, J=12.1 Hz, 2H), 2.61 (t, J=10.7 Hz, 2H), 2.43 (s, 3H), 1.72 (dd, J=13.6, 8.7 Hz, 4H).

### Example 108: Synthesis of Compound 394

### Synthesis of Intermediate C54

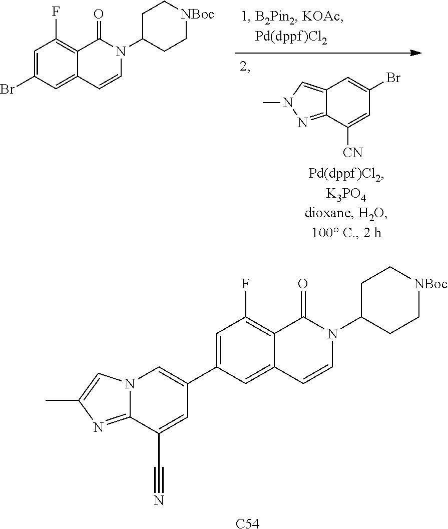 [SMILES: CC(C)(C)OC(=O)N1CCC(n2ccc3cc(Br)cc(F)c3c2=O)CC1 ; Cn1cc2cc(Br)cc(C#N)c2n1 ; Cc1cn2cc(-c3cc(F)c4c(=O)n(C5CCN(C(=O)OC(C)(C)C)CC5)ccc4c3)cc(C#N)c2n1 ; "C54"]

To a stirred solution of tert-butyl 4-(6-bromo-8-fluoro-1-oxoisoquinolin-2-yl)piperidine-1-carboxylate (45 mg, 0.106 mmol, 1 equiv) and bis(pinacolato)diboron (32 mg, 0.127 mmol, 1.2 equiv) in dioxane (5 mL) was added potassium acetate (21 mg, 0.212 mmol, 2 equiv), and Pd(dppf)Cl₂ (8 mg, 0.011 mmol, 0.1 equiv) in portions at room temperature under a nitrogen atmosphere. The resulting mixture was stirred for 2 hr at 100° C. under a nitrogen atmosphere, then cooled to room temperature. To the reaction mixture was added 5-bromo-2-methylindazole-7-carbonitrile (25 mg, 0.106 mmol, 1 equiv), K₃PO₄ (45 mg, 0.212 mmol, 2 equiv), H₂O (1 mL), and Pd(dppf)Cl₂ (8 mg, 0.011 mmol, 0.1 equiv) in portions at room temperature. The resulting mixture was stirred for additional 2 hr at 100° C., then cooled to room temperature, quenched with water (10 mL) and extracted with ethyl acetate (3×3 mL). The organic layers were combined, washed with brine (1×10 mL), dried over anhydrous Na₂SO₄, and filtered. After filtration, the filtrate was concentrated under reduced pressure to give a residue. The residue was purified by reverse flash chromatography to afford tert-butyl 4-[6-(7-cyano-2-methylindazol-5-yl)-8-fluoro-1-oxoisoquinolin-2-yl]piperidine-1-carboxylate (C54, 32 mg, 60%) as a solid. LCMS (ES, m/z): 502 [M+H]

### Synthesis of Compound 394

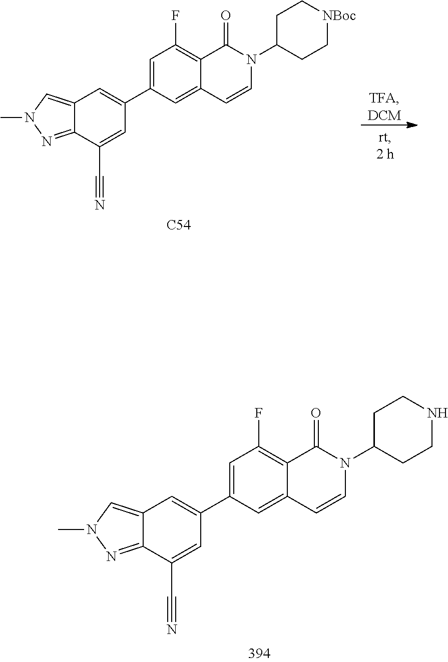 [SMILES: Cn1cc2cc(-c3cc(F)c4c(=O)n(C5CCN(C(=O)OC(C)(C)C)CC5)ccc4c3)cc(C#N)c2n1 ; "C54" ; Cn1cc2cc(-c3cc(F)c4c(=O)n(C5CCNCC5)ccc4c3)cc(C#N)c2n1]

A solution of tert-butyl 4-(6-(7-cyano-2-methyl-2H-indazol-5-yl)-8-fluoro-1-oxoisoquinolin-2(1H)-yl)piperidine-1-carboxylate (30 mg, 0.058 mmol, 1 equiv) in trifluoroacetic acid (1 mL) and dichloromethane (1 mL) was stirred for 2 hr at room temperature. The resulting mixture was concentrated under reduced pressure to give a residue. The residue was basified to pH 8 with 7 M NH₃(g) in methanol. The resulting mixture was concentrated under vacuum to give a residue. The residue was purified by reverse flash chromatography to afford 5-(8-fluoro-1-oxo-2-(piperidin-4-yl)-1,2-dihydroisoquinolin-6-yl)-2-methyl-2H-indazole-7-carbonitrile (Compound 394, 15 mg, 62%) as a solid. LCMS (ES, m/z): 402 [M+H]^{+ 1}H NMR (300 MHz, DMSO-d₆) δ 8.75 (s, 2H), 8.63 (s, 1H), 8.50 (s, 1H), 8.42 (s, 1H), 7.97 (s, 1H), 7.74 (d, J=13.4 Hz, 1H), 7.42 (d, J=7.5 Hz, 1H), 6.76 (d, J=7.4 Hz, 1H), 5.04 (s, 1H), 4.12 (s, 3H), 3.47 (d, J=12.4 Hz, 2H), 3.17 (d, J=12.2 Hz, 2H), 2.21-2.03 (m, 2H), 1.99 (d, J=13.0 Hz, 2H).

### Example 109: Synthesis of Compound 400

### Synthesis of Intermediate C55

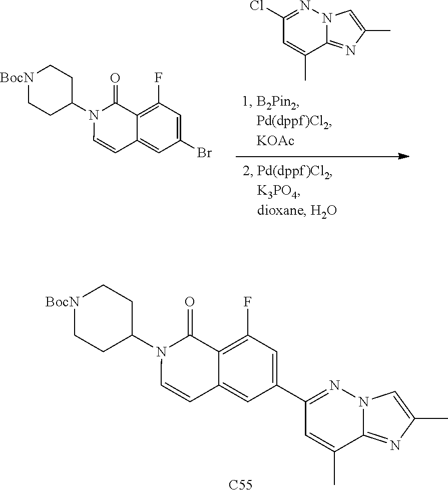 [SMILES: CC(C)(C)OC(=O)N1CCC(n2ccc3cc(Br)cc(F)c3c2=O)CC1 ; Cc1cn2nc(Cl)cc(C)c2n1 ; "C55" ; Cc1cn2nc(-c3cc(F)c4c(=O)n(C5CCN(C(=O)OC(C)(C)C)CC5)ccc4c3)cc(C)c2n1]

To a stirred solution of tert-butyl 4-(6-bromo-8-fluoro-1-oxoisoquinolin-2-yl)piperidine-1-carboxylate (130 mg, 0.306 mmol, 1 equiv) and bis(pinacolato)diboron (93 mg, 0.367 mmol, 1.2 equiv) in dioxane (5 mL) was added potassium acetate (60 mg, 0.612 mmol, 2 equiv) and Pd(dppf)Cl₂ (23 mg, 0.031 mmol, 0.1 equiv) in portions at room temperature under a nitrogen atmosphere. The resulting mixture was stirred for 3 hr at 100° C. under a nitrogen atmosphere, then cooled to room temperature. To the reaction mixture was added 6-chloro-2,8-dimethylimidazo[1,2-b]pyridazine (56 mg, 0.306 mmol, 1 equiv), K₃PO₄ (130 mg, 0.612 mmol, 2 equiv), H₂O (1 mL), and Pd(dppf)Cl₂ (23 mg, 0.031 mmol, 0.1 equiv) in portions at room temperature. The resulting mixture was stirred for an additional 3 hr at 100° C., then cooled to room temperature, quenched with water (10 mL) and extracted with ethyl acetate (3×10 mL). The organic layers were combined, washed with brine (1×10 mL), dried over anhydrous Na₂SO₄, and filtered. After filtration, the filtrate was concentrated under reduced pressure to give a residue. The residue was purified by reverse flash chromatography to afford tert-butyl 4-(6-{2,8-dimethylimidazo[1,2-b]pyridazin-6-yl}-8-fluoro-1-oxoisoquinolin-2-yl)piperidine-1-carboxylate (C55, 90 mg, 59%) as a solid. LCMS (ES, m/z): 492 [M+H]⁺.

### Synthesis of Intermediate C56

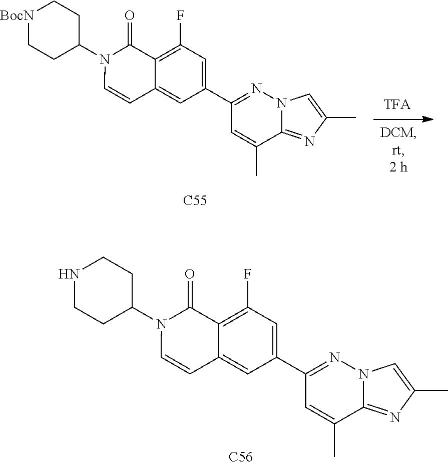 [SMILES: Cc1cn2nc(-c3cc(F)c4c(=O)n(C5CCN(C(=O)OC(C)(C)C)CC5)ccc4c3)cc(C)c2n1 ; "C55" ; "C56" ; Cc1cn2nc(-c3cc(F)c4c(=O)n(C5CCNCC5)ccc4c3)cc(C)c2n1]

A solution of tert-butyl 4-(6-{2,8-dimethylimidazo[1,2-b]pyridazin-6-yl}-8-fluoro-1-oxoisoquinolin-2-yl)piperidine-1-carboxylate (90 mg, 0.183 mmol, 1 equiv) in trifluoroacetic acid (2 mL) and dichloromethane (2 mL) was stirred for 2 hr at room temperature. The resulting mixture was concentrated under reduced pressure to give a residue. The residue was basified to pH 8 with 7 M NH₃ (g) in methanol. The resulting mixture was concentrated under reduced pressure to give a residue. The residue was purified by reverse flash chromatography to afford 6-{2,8-dimethylimidazo[1,2-b]pyridazin-6-yl}-8-fluoro-2-(piperidin-4-yl)isoquinolin-1-one (C56, 68 mg, 94%) as a solid. LCMS (ES, m/z): 392 [M+H]⁺.

### Synthesis of Compound 400

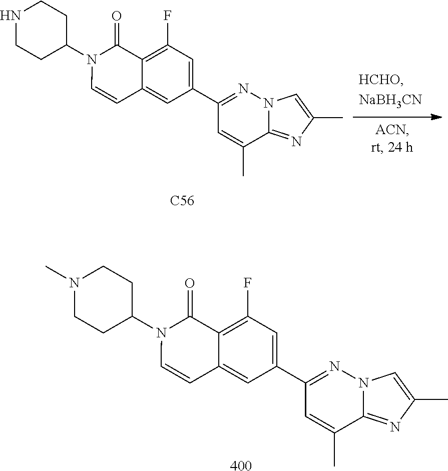

To a stirred solution of 6-{2,8-dimethylimidazo[1,2-b]pyridazin-6-yl}-8-fluoro-2-(piperidin-4-yl)isoquinolin-1-one (32 mg, 0.082 mmol, 1 equiv) and HCHO (13 mg, 0.410 mmol, 5 equiv) in acetonitrile (5 mL) was added NaBH₃CN (16 mg, 0.246 mmol, 3 equiv) in portions at room temperature. The resulting mixture was stirred for 30 min at room temperature. To the reaction mixture was added HOAc (49 mg, 0.820 mmol, 10 equiv) dropwise at room temperature. The resulting mixture was stirred for an additional 2 hr at room temperature, then quenched with water (10 mL), basified to pH 8 with saturated NaHCO₃ (aq.), and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by reverse flash chromatography to afford 6-(2,8-dimethylimidazo[1,2-b]pyridazin-6-yl)-8-fluoro-2-(1-methylpiperidin-4-yl)isoquinolin-1(2H)-one (Compound 400, 10.2 mg, 30%) as a solid. LCMS (ES, m/z): 406 [M+H]^{+ 1}H NMR (300 MHz, Methanol-d₄) δ 8.39 (dd, J=3.0, 1.3 Hz, 2H), 8.27 (d, J=1.7 Hz, 1H), 7.99 (dd, J=12.8, 1.7 Hz, 1H), 7.55 (d, J=7.6 Hz, 1H), 6.92 (dd, J=7.6, 2.0 Hz, 1H), 5.11 (t, J=12.3 Hz, 1H), 3.73 (d, J=12.5 Hz, 2H), 2.98 (s, 3H), 2.83 (d, J=1.1 Hz, 3H), 2.69 (d, J=1.0 Hz, 3H), 2.53-2.34 (m, 2H), 2.22 (d, J=13.5 Hz, 2H).

### Example 110: Synthesis of Compound 110

### Synthesis of Intermediate C57

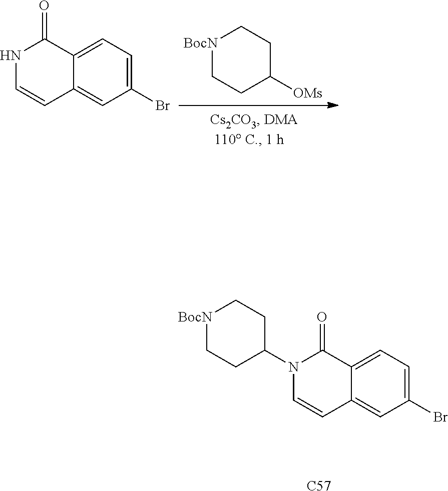 [SMILES: O=c1[nH]ccc2cc(Br)ccc12 ; CC(C)(C)OC(=O)N1CCC(OS(C)(=O)=O)CC1 ; CC(C)(C)OC(=O)N1CCC(n2ccc3cc(Br)ccc3c2=O)CC1 ; "C57"]

To a stirred solution of 6-bromo-2H-isoquinolin-1-one (3 g, 13.389 mmol, 1 equiv) and tert-butyl 4-(methanesulfonyloxy)piperidine-1-carboxylate (5.61 g, 20.084 mmol, 1.5 equiv) in DMA (60 mL) was added Cs₂CO₃ (13.09 g, 40.167 mmol, 3.0 equiv) in portions at room temperature. The resulting mixture was stirred for 1 h at 110° C. The resulting mixture was diluted with H₂O (30 mL) and extracted with ethyl acetate (2×50 mL). The organic layers were combined, washed brine (1×10 mL), dried over anhydrous Na₂SO₄, and filtered. After filtration, the filtrate was concentrated under reduced pressure to give a residue. The residue was purified by reverse flash chromatography (Condition 1, Gradient 1) to afford tert-butyl 4-(6-bromo-1-oxoisoquinolin-2-yl)piperidine-1-carboxylate (2 g, 37%) as an oil. LCMS (ES, m/z): 407 [M+H]⁺.

### Synthesis of Intermediate C58

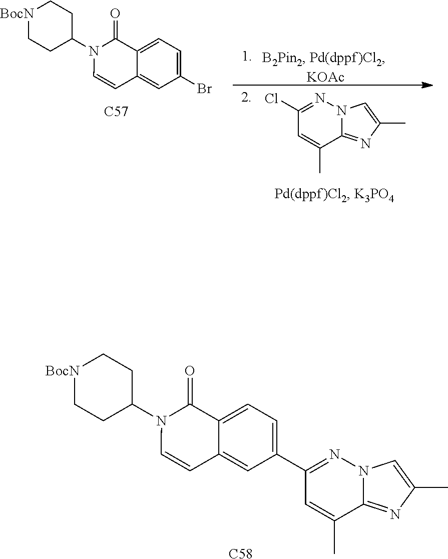 [SMILES: CC(C)(C)OC(=O)N1CCC(n2ccc3cc(Br)ccc3c2=O)CC1 ; "C57" ; Cc1cn2nc(Cl)cc(C)c2n1 ; "C58" ; Cc1cn2nc(-c3ccc4c(=O)n(C5CCN(C(=O)OC(C)(C)C)CC5)ccc4c3)cc(C)c2n1]

To a mixture of tert-butyl 4-(6-bromo-1-oxoisoquinolin-2-yl)piperidine-1-carboxylate (1.0 g, 2.455 mmol, 1 equiv), bis(pinacolato)diboron (0.75 g, 2.946 mmol, 1.2 equiv), and KOAc (0.48 g, 4.910 mmol, 2.0 equiv) in dioxane (25 mL) was added Pd(dppf)Cl₂CH₂Cl₂ (0.20 g, 0.246 mmol, 0.1 equiv) at room temperature under nitrogen atmosphere. After stirring for 1 h at 80° C. under a nitrogen atmosphere, the resulting mixture was allowed to cool to room temperature. To the reaction mixture was added 6-chloro-2,8-dimethylimidazo[1,2-b]pyridazine (0.49 g, 2.701 mmol, 1.1 equiv), K₃PO₄ (1.56 g, 7.365 mmol, 3.0 equiv), Pd(dppf)Cl₂CH₂Cl₂ (0.20 g, 0.246 mmol, 0.1 equiv), and H₂O (5 mL). After stirring for an additional 3 h at 80° C. under a nitrogen atmosphere, the resulting mixture was concentrated under reduced pressure to give a residue. The residue was purified by silica gel column chromatography, eluted with EA:PE (9:1) to afford tert-butyl 4-(6-{2,8-dimethylimidazo[1,2-b]pyridazin-6-yl}-1-oxoisoquinolin-2-yl)piperidine-1-carboxylate (600 mg, 52%) as a solid. LCMS (ES, m/z): 474 [M+H]⁺.

### Synthesis of Intermediate C59

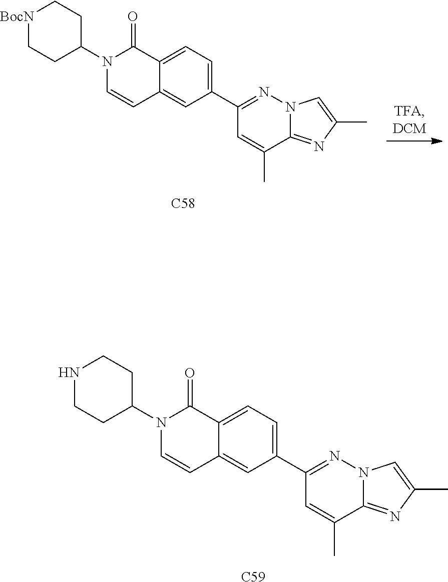 [SMILES: Cc1cn2nc(-c3ccc4c(=O)n(C5CCN(C(=O)OC(C)(C)C)CC5)ccc4c3)cc(C)c2n1 ; "C58" ; Cc1cn2nc(-c3ccc4c(=O)n(C5CCNCC5)ccc4c3)cc(C)c2n1 ; "C59"]

A solution of tert-butyl 4-(6-{2,8-dimethylimidazo[1,2-b]pyridazin-6-yl}-1-oxoisoquinolin-2-yl)piperidine-1-carboxylate (380 mg, 0.802 mmol, 1 equiv) and TFA (3 mL) in DCM (9 mL) was stirred for 1 h at room temperature. The resulting mixture was concentrated under reduced pressure to give a residue. The residue was purified by reverse flash chromatography (Condition 2, Gradient 1) to afford 6-{2,8-dimethylimidazo[1,2-b]pyridazin-6-yl}-2-(piperidin-4-yl)isoquinolin-1-one (200 mg, 67%) as a solid. LCMS (ES, m/z): 374 [M+H]⁺.

### Synthesis of Compound 309

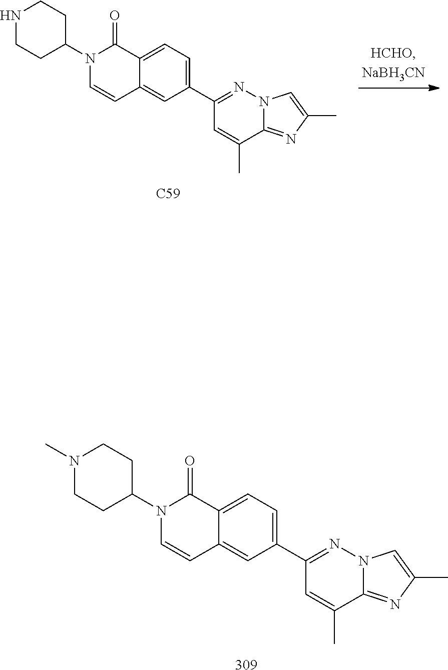

To a stirred solution of 6-{2,8-dimethylimidazo[1,2-b]pyridazin-6-yl}-2-(piperidin-4-yl)isoquinolin-1-one (80 mg, 0.214 mmol, 1 equiv) in methanol (2.5 mL) was added formaldehyde (173 mg, 2.140 mmol, 10 equiv, 37% aq) and NaBH₃CN (80.77 mg, 1.284 mmol, 6 equiv) in portions at room temperature. The resulting mixture was stirred for 12 h at room temperature, then concentrated under reduced pressure to give a residue. The residue was purified by reverse flash chromatography (Condition 2, Gradient 2) to afford 6-{2,8-dimethylimidazo[1,2-b]pyridazin-6-yl}-2-(1-methylpiperidin-4-yl)isoquinolin-1-one (30 mg, 36%) as a solid. LCMS (ES, m/z): 387 [M+H]⁺. ¹H NMR (300 MHz, DMSO-d₆) δ 8.41-8.31 (m, 2H), 8.20-8.08 (m, 2H), 7.77 (d, J=1.2 Hz, 1H), 7.62 (d, J=7.6 Hz, 1H), 6.79 (d, J=7.5 Hz, 1H), 4.82 (s, 1H), 2.98-2.87 (m, 2H), 2.64 (d, J=1.0 Hz, 3H), 2.43 (d, J=0.8 Hz, 3H), 2.24 (s, 3H), 2.16-1.88 (m, 3H), 1.73 (d, J=11.0 Hz, 2H).

### Example 111: Synthesis of Compound 402

### Synthesis of Compound 402

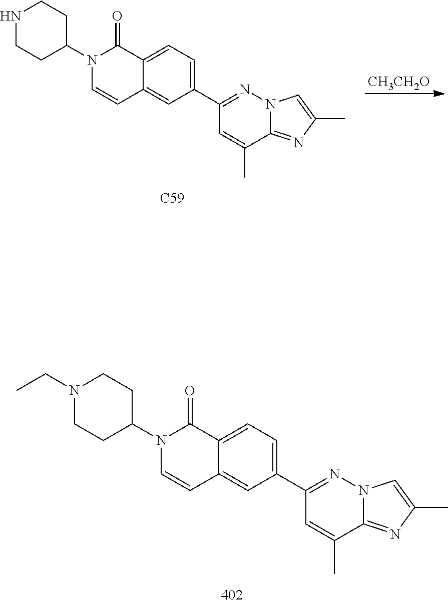

To a stirred solution of 6-{2,8-dimethylimidazo[1,2-b]pyridazin-6-yl}-2-(piperidin-4-yl)isoquinolin-1-one (50 mg, 0.134 mmol, 1 equiv) in methanol (2.5 mL) was added acetaldehyde (58.98 mg, 1.340 mmol, 10 equiv) and NaBH₃CN (50.48 mg, 0.804 mmol, 6.0 equiv) in portions at room temperature. The resulting mixture was stirred for 1 h at room temperature, then concentrated under reduced pressure to give a residue. The residue was purified by reverse flash chromatography (Condition 2, Gradient 3) to afford 6-{2,8-dimethylimidazo[1,2-b]pyridazin-6-yl}-2-(1-ethylpiperidin-4-yl)isoquinolin-1-one (13 mg, 24%) as a solid. LCMS (ES, m/z): 402 [M+H]⁺. ¹H NMR (300 MHz, DMSO-d₆) δ 8.41-8.31 (m, 2H), 8.20-8.09 (m, 2H), 7.76 (d, J=1.2 Hz, 1H), 7.64 (d, J=7.6 Hz, 1H), 6.78 (d, J=7.6 Hz, 1H), 4.82 (t, J=12.0 Hz, 1H), 3.04 (d, J=10.5 Hz, 2H), 2.64 (d, J=1.1 Hz, 3H), 2.46-2.33 (m, 5H), 2.06 (t, J=11.2 Hz, 2H), 1.99-1.86 (m, 2H), 1.75 (d, J=11.9 Hz, 2H), 1.04 (t, J=7.1 Hz, 3H).

### Example 112: Synthesis of Compounds 395 and 396

### Synthesis of Intermediate C60

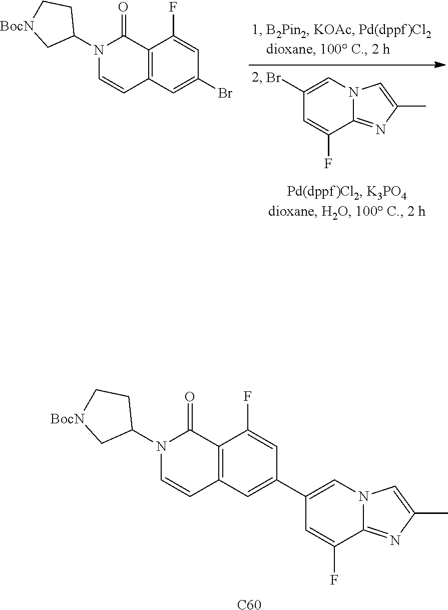 [SMILES: CC(C)(C)OC(=O)N1CCC(n2ccc3cc(Br)cc(F)c3c2=O)C1 ; Cc1cn2cc(Br)cc(F)c2n1 ; Cc1cn2cc(-c3cc(F)c4c(=O)n(C5CCN(C(=O)OC(C)(C)C)C5)ccc4c3)cc(F)c2n1 ; "C60"]

To a stirred mixture of tert-butyl 3-(6-bromo-8-fluoro-1-oxoisoquinolin-2-yl)pyrrolidine-1-carboxylate (180 mg, 0.438 mmol, 1 equiv) and bis(pinacolato)diboron (133 mg, 0.526 mmol, 1.2 equiv) in dioxane (10 mL) was added KOAc (86 mg, 0.876 mmol, 2 equiv) and Pd(dppf)Cl₂ (32 mg, 0.044 mmol, 0.1 equiv) in portions at room temperature under nitrogen atmosphere. The resulting mixture was stirred for 2 h at 100° C. under nitrogen atmosphere, then cooled to room temperature. To the resulting mixture was added 6-bromo-8-fluoro-2-methylimidazo[1,2-a]pyridine (100 mg, 0.438 mmol, 1 equiv), K₃PO₄ (186 mg, 0.876 mmol, 2 equiv), H₂O (2 mL), and Pd(dppf)Cl₂ (32 mg, 0.044 mmol, 0.1 equiv) in portions at room temperature. The resulting mixture was stirred for an additional 2 h at 100° C., then cooled to room temperature, and quenched with water (10 mL). The resulting mixture was extracted with ethyl acetate (3×3 mL). The organic layers were combined, washed with brine (1×10 mL), dried over anhydrous Na₂SO₄, and filtered. After filtration, the filtrate was concentrated under reduced pressure to give a residue. The residue was purified by reverse flash chromatography (Condition 2, Gradient 4) to afford tert-butyl 3-(8-fluoro-6-{8-fluoro-2-methylimidazo[1,2-a]pyridin-6-yl}-1-oxoisoquinolin-2-yl)pyrrolidine-1-carboxylate (165 mg, 78%) as a solid. LCMS (ES, m/z): 481 [M+H]⁺

### Synthesis of Compounds 395 and 396

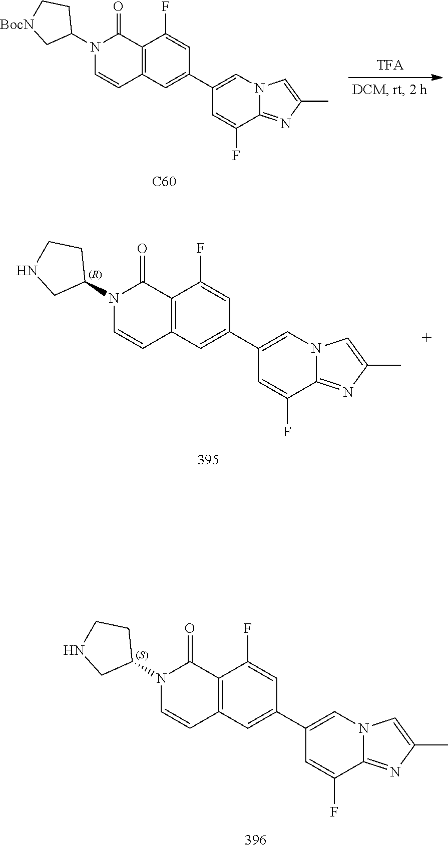

A solution of tert-butyl 3-(8-fluoro-6-{8-fluoro-2-methylimidazo[1,2-a]pyridin-6-yl}-1-oxoisoquinolin-2-yl)pyrrolidine-1-carboxylate (80 mg, 0.166 mmol, 1 equiv) in TFA (1 mL) and DCM (1 mL) was stirred for 2 h at room temperature. The resulting mixture was concentrated under reduced pressure to give a residue. The residue was basified to pH 10 with 7 M NH₃ in methanol. The resulting mixture was concentrated under reduced pressure to give a residue. The residue was purified by reverse flash chromatography (Condition 2, Gradient 5), followed by chiral prep-HPLC (Condition 2, Gradient 1) to afford (R)-8-fluoro-6-(8-fluoro-2-methylimidazo[1,2-a]pyridin-6-yl)-2-(pyrrolidin-3-yl)isoquinolin-1(2H)-one (6.9 mg, 11%) and (S)-8-fluoro-6-(8-fluoro-2-methylimidazo[1,2-a]pyridin-6-yl)-2-(pyrrolidin-3-yl)isoquinolin-1(2H)-one (6.1 mg, 10%) as solids. First Peak (395) RT=7.798 min. LCMS (ES, m/z): 381 [M+H]⁺. ¹H NMR (300 MHz, DMSO-d₆) δ 9.00 (s, 1H), 7.90-7.80 (m, 2H), 7.73-7.59 (m, 3H), 6.69 (s, 1H), 5.34 (d, J=8.7 Hz, 1H), 3.11 (td, J=16.5, 14.1, 7.1 Hz, 2H), 2.84 (dd, J=11.6, 4.7 Hz, 2H), 2.39 (s, 4H), 1.80-1.70 (m, 1H). Second Peak (396) RT=9.126 min. LCMS (ES, m/z): 381 [M+H]⁺. ¹H NMR (300 MHz, DMSO-d₆) δ 9.00 (s, 1H), 7.90-7.80 (m, 2H), 7.73-7.59 (m, 3H), 6.69 (s, 1H), 5.34 (d, J=8.7 Hz, 1H), 3.11 (td, J=16.5, 14.1, 7.1 Hz, 2H), 2.84 (dd, J=11.6, 4.7 Hz, 2H), 2.39 (s, 4H), 1.80-1.70 (m, 1H).

### Example 113: Synthesis of Compounds 403 and 404

### Synthesis of Intermediate C61

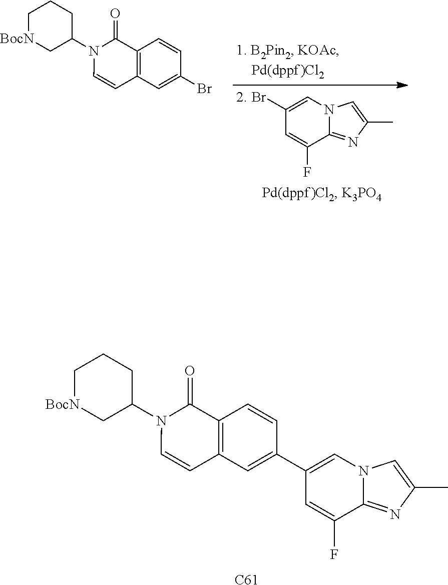 [SMILES: CC(C)(C)OC(=O)N1CCCC(n2ccc3cc(Br)ccc3c2=O)C1 ; Cc1cn2cc(Br)cc(F)c2n1 ; Cc1cn2cc(-c3ccc4c(=O)n(C5CCCN(C(=O)OC(C)(C)C)C5)ccc4c3)cc(F)c2n1 ; "C61"]

To a mixture of tert-butyl 3-(6-bromo-1-oxoisoquinolin-2-yl)piperidine-1-carboxylate (100 mg, 0.246 mmol, 1 equiv) and 4,4,5,5-tetramethyl-2-(tetramethyl-1,3,2-dioxaborolan-2-yl)-1,3,2-dioxaborolane (75 mg, 0.295 mmol, 1.2 equiv) in dioxane (2.5 mL) was added KOAc (48 mg, 0.492 mmol, 2.0 equiv) and Pd(dppf)Cl₂·CH₂Cl₂ (20 mg, 0.025 mmol, 0.1 equiv) at room temperature under nitrogen atmosphere. After stirring for 1 h at 80° C. under a nitrogen atmosphere, the reaction mixture was cooled to room temperature. To the resulting mixture was added 6-bromo-8-fluoro-2-methylimidazo[1,2-a]pyridine (68 mg, 0.295 mmol, 1.2 equiv), K₃PO₄ (156 mg, 0.738 mmol, 3.0 equiv), Pd(dppf)Cl₂·CH₂Cl₂ (20 mg, 0.025 mmol, 0.1 equiv), and H₂O (0.5 mL). After stirring for 2 h at 80° C. under a nitrogen atmosphere, the resulting mixture was concentrated under reduced pressure to give a residue. The residue was purified by reverse flash chromatography (Condition 2, Gradient 1) to afford tert-butyl 3-(6-{8-fluoro-2-methylimidazo[1,2-a]pyridin-6-yl}-1-oxoisoquinolin-2-yl)piperidine-1-carboxylate (110 mg, 94%) as a solid. LCMS (ES, m/z): 477 [M+H]⁺.

### Synthesis of Compounds 403 and 404

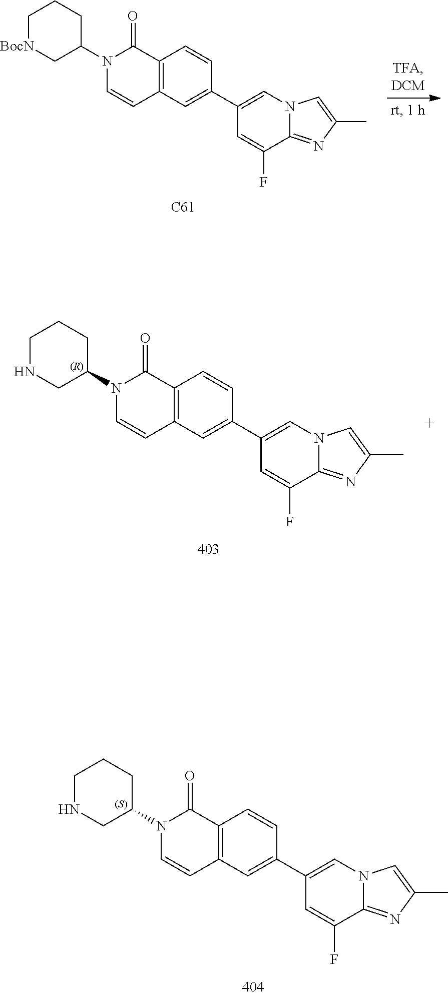

A mixture of tert-butyl 3-(6-{8-fluoro-2-methylimidazo[1,2-a]pyridin-6-yl}-1-oxoisoquinolin-2-yl)piperidine-1-carboxylate (100 mg, 0.210 mmol, 1 equiv) and TFA (0.5 mL) in DCM (2 mL) was stirred for 1 h at room temperature. The resulting mixture was concentrated under reduced pressure to give a residue. The residue was purified by reverse flash chromatography (Condition 2, Gradient 6), followed by chiral HPLC (Condition 3, Gradient 1) to afford 6-{8-fluoro-2-methylimidazo[1,2-a]pyridin-6-yl}-2-[(3R)-piperidin-3-yl]isoquinolin-1-one (13 mg, 13%) and 6-{8-fluoro-2-methylimidazo[1,2-a]pyridin-6-yl}-2-[(3S)-piperidin-3-yl]isoquinolin-1-one (15 mg, 19%) as solids. First Peak (403) RT=6.742 min. LCMS (ES, m/z):377 [M+H]⁺. ¹H NMR (300 MHz, DMSO-d₆) δ 8.94 (d, J=1.5 Hz, 1H), 8.31 (d, J=8.5 Hz, 1H), 8.03 (d, J=1.8 Hz, 1H), 7.90-7.81 (m, 2H), 7.69-7.58 (m, 2H), 6.69 (d, J=7.5 Hz, 1H), 4.88 (s, 1H), 2.94 (d, J=14.5 Hz, 2H), 2.80 (d, J=11.2 Hz, 1H), 2.39 (s, 3H), 1.87 (s, 2H), 1.78 (d, J=13.8 Hz, 1H), 1.60 (s, 1H). Second Peak (404) RT=9.418. LCMS (ES, m/z):377 [M+H]⁺. ¹H NMR (300 MHz, DMSO-d₆) δ 8.94 (d, J=1.5 Hz, 1H), 8.31 (d, J=8.5 Hz, 1H), 8.03 (d, J=1.8 Hz, 1H), 7.90-7.81 (m, 2H), 7.69-7.58 (m, 2H), 6.69 (d, J=7.5 Hz, 1H), 4.88 (s, 1H), 2.94 (d, J=14.5 Hz, 2H), 2.80 (d, J=11.2 Hz, 1H), 2.39 (s, 3H), 1.87 (s, 2H), 1.78 (d, J=13.8 Hz, 1H), 1.60 (s, 1H).

### Example 114: Synthesis of Compound 471

### Synthesis of Intermediate C62

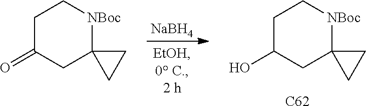

To a stirred solution of tert-butyl 7-oxo-4-azaspiro[2.5]octane-4-carboxylate (10 g, 44.388 mmol, 1 equiv) in ethanol (150 mL) was added NaBH₄ (2.5 g, 66.582 mmol, 1.5 equiv) in portions at 0° C. under nitrogen atmosphere. The resulting mixture was stirred for 2 h at room temperature under nitrogen atmosphere, then concentrated under vacuum to give a residue. The residue was dissolved in dichloromethane (20 mL). To the resulting mixture was added H₂O (50 mL). The aqueous layer was extracted with dichloromethane (3×20 mL). The organic layers were combined, washed with brine (1×50 mL), dried over anhydrous Na₂SO₄, and filtered. After filtration, the filtrate was concentrated under reduced pressure to afford tert-butyl 7-hydroxy-4-azaspiro[2.5]octane-4-carboxylate (10 g, 99%) as an oil. ¹H NMR (300 MHz, DMSO-d₆) δ 4.66 (d, J=4.9 Hz, 1H), 3.73 (tt, J=14.9, 4.4 Hz, 2H), 2.82 (t, J=12.5 Hz, 1H), 1.85-1.66 (m, 1H), 1.62-1.50 (m, 1H), 1.40 (s, 9H), 1.29-1.15 (m, 2H), 1.05-0.93 (m, 1H), 0.74 (ddd, J=9.5, 6.7, 5.5 Hz, 1H), 0.52-0.27 (m, 2H).

### Synthesis of Intermediate C63

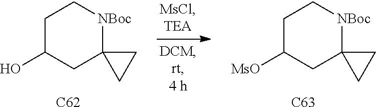 [SMILES: CC(C)(C)OC(=O)N1CCC(O)CC12CC2 ; CC(C)(C)OC(=O)N1CCC(OS(C)(=O)=O)CC12CC2 ; "C62" ; "C63"]

To a stirred mixture of tert-butyl 7-hydroxy-4-azaspiro[2.5]octane-4-carboxylate (11.1 g, 48.833 mmol, 1 equiv) and TEA (9.9 g, 97.666 mmol, 2 equiv) in DCM (120 mL) was added MsCl (8.4 g, 73.249 mmol, 1.5 equiv) dropwise at 0° C. under nitrogen atmosphere. The resulting mixture was stirred for 4 h at room temperature under nitrogen atmosphere, then concentrated under reduced pressure to afford tert-butyl 7-(methanesulfonyloxy)-4-azaspiro[2.5]octane-4-carboxylate (14.5 g, 97%) as an oil. ¹H NMR (300 MHz, DMSO-d₆) δ 4.89 (tt, J=9.0, 4.5 Hz, 1H), 3.71 (dt, J=13.9, 4.4 Hz, 1H), 3.20 (s, 3H), 3.10 (t, J=11.5 Hz, 1H), 2.05-1.93 (m, 1H), 1.83-1.43 (m, 3H), 1.41 (s, 9H), 0.98 (hept, J=7.2 Hz, 1H), 0.81 (ddd, J=9.7, 6.7, 5.4 Hz, 1H), 0.62 (ddd, J=9.6, 7.4, 2.9 Hz, 2H).

### Synthesis of Intermediate C64

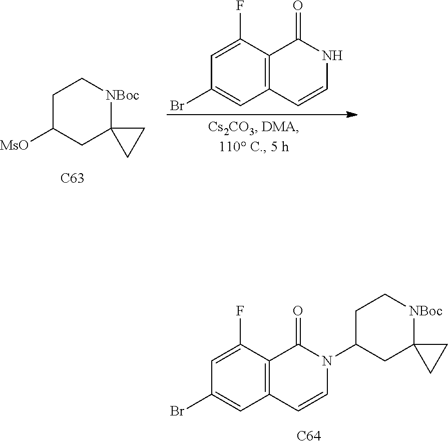

To a stirred mixture of 6-bromo-8-fluoro-2H-isoquinolin-1-one (4 g, 16.526 mmol, 1 equiv) and tert-butyl 7-(methanesulfonyloxy)-4-azaspiro[2.5]octane-4-carboxylate (6.1 g, 19.831 mmol, 1.2 equiv) in DMA (40 mL) was added Cs₂CO₃ (16.2 g, 49.578 mmol, 3 equiv) in portions at room temperature. The resulting mixture was stirred for 5 h at 100° C., then cooled to room temperature. The resulting mixture was concentrated under reduced pressure to give a residue. The residue was purified by reverse flash chromatography (Condition 2, Gradient 7) to afford tert-butyl 7-(6-bromo-8-fluoro-1-oxoisoquinolin-2-yl)-4-azaspiro[2.5]octane-4-carboxylate (1.4 g, 19%) as a solid. LCMS (ES, m/z): 451 [M+H]⁺.

### Synthesis of Intermediate C65

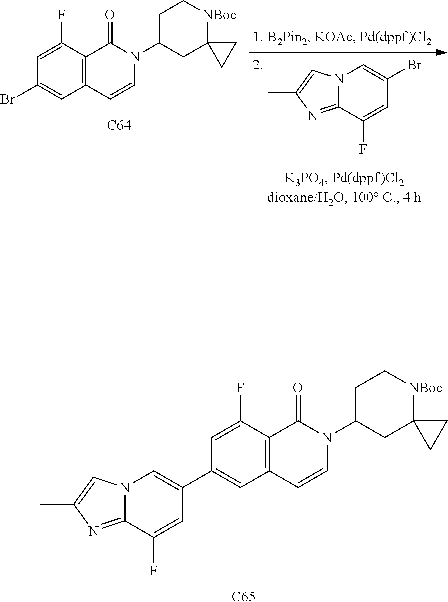 [SMILES: "C64" ; CC(C)(C)OC(=O)N1CCC(n2ccc3cc(Br)cc(F)c3c2=O)CC12CC2 ; Cc1cn2cc(Br)cc(F)c2n1 ; Cc1cn2cc(-c3cc(F)c4c(=O)n(C5CCN(C(=O)OC(C)(C)C)C6(CC6)C5)ccc4c3)cc(F)c2n1 ; "C65"]

To a stirred mixture of tert-butyl 7-(6-bromo-8-fluoro-1-oxoisoquinolin-2-yl)-4-azaspiro[2.5]octane-4-carboxylate (100 mg, 0.222 mmol, 1 equiv) and bis(pinacolato)diboron (68 mg, 0.266 mmol, 1.2 equiv) in dioxane (5 mL) was added KOAc (44 mg, 0.444 mmol, 2 equiv) and Pd(dppf)Cl₂ (16 mg, 0.022 mmol, 0.1 equiv) in portions at room temperature under nitrogen atmosphere. The resulting mixture was stirred for 3 h at 100° C. under nitrogen atmosphere, then cooled to room temperature. To the resulting mixture was added 5-bromo-7-fluoro-2-methylindazole (51 mg, 0.222 mmol, 1 equiv), H₂O (1 mL), K₃PO₄ (94 mg, 0.444 mmol, 2 equiv), and Pd(dppf)Cl₂ (16 mg, 0.022 mmol, 0.1 equiv) in portions at room temperature. The reaction mixture was stirred for additional 3 h at 100° C., then cooled to room temperature. The resulting mixture was diluted with H₂O (10 mL) and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 7-[8-fluoro-6-(7-fluoro-2-methylindazol-5-yl)-1-oxoisoquinolin-2-yl]-4-azaspiro[2.5]octane-4-carboxylate (30 mg, 26%) as a solid. LCMS (ES, m/z): 521 [M+H]⁺.

### Synthesis of Compound 471

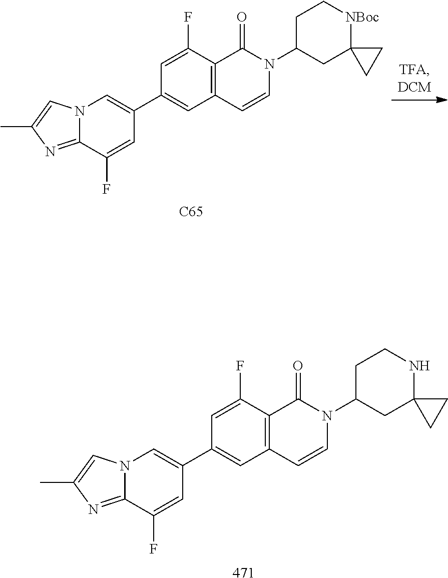 [SMILES: Cc1cn2cc(-c3cc(F)c4c(=O)n(C5CCN(C(=O)OC(C)(C)C)C6(CC6)C5)ccc4c3)cc(F)c2n1 ; "C65" ; Cc1cn2cc(-c3cc(F)c4c(=O)n(C5CCNC6(CC6)C5)ccc4c3)cc(F)c2n1 ; "471"]

A solution of tert-butyl 7-(8-fluoro-6-{8-fluoro-2-methylimidazo[1,2-a]pyridin-6-yl}-1-oxoisoquinolin-2-yl)-4-azaspiro[2.5]octane-4-carboxylate (25 mg, 0.048 mmol, 1 equiv) in TFA (1 mL) and DCM (1 mL) was stirred for 3 h at room temperature. The resulting mixture was concentrated under reduced pressure to give a residue. The residue was basified to pH 8 with 7 M NH₃ in methanol. The resulting mixture was concentrated under vacuum to give a residue. The residue was purified by reverse flash chromatography (Condition 2, Gradient 4) to afford 2-{4-azaspiro[2.5]octan-7-yl}-8-fluoro-6-{8-fluoro-2-methylimidazo[1,2-a]pyridin-6-yl}isoquinolin-1-one (16 mg, 79%) as a solid. LCMS (ES, m/z): 421 [M+H]⁺. ¹H NMR (300 MHz, DMSO-d₆) δ 8.60 (d, J=2.8 Hz, 1H), 8.08 (d, J=1.4 Hz, 1H), 7.87 (d, J=1.8 Hz, 1H), 7.64 (dd, J=13.6, 1.7 Hz, 1H), 7.63-7.51 (m, 2H), 6.71 (dd, J=7.6, 2.1 Hz, 1H), 5.04 (d, J=7.9 Hz, 1H), 4.24 (s, 3H), 3.03 (d, J=12.7 Hz, 1H), 2.73 (s, 1H), 2.20 (t, J=12.0 Hz, 1H), 1.75 (s, 2H), 1.14 (dd, J=11.8, 3.9 Hz, 1H), 0.60 (t, J=6.3 Hz, 1H), 0.43 (dd, J=23.4, 6.7 Hz, 2H), 0.42 (s, 1H).

### Example 115: Synthesis of Compound 415

### Synthesis of Intermediate C66

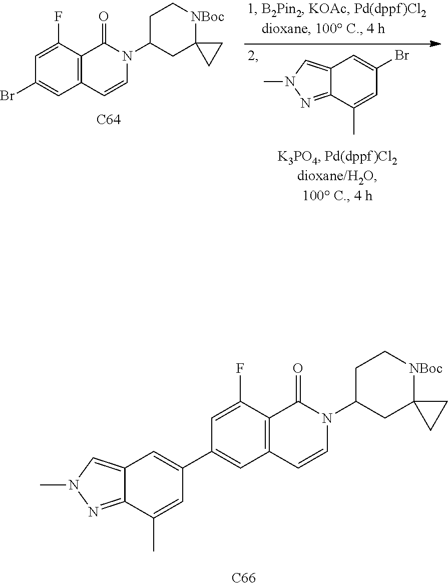 [SMILES: "C64" ; CC(C)(C)OC(=O)N1CCC(n2ccc3cc(Br)cc(F)c3c2=O)CC12CC2 ; Cc1cc(Br)cc2cn(C)nc12 ; Cc1cc(-c2cc(F)c3c(=O)n(C4CCN(C(=O)OC(C)(C)C)C5(CC5)C4)ccc3c2)cc2cn(C)nc12 ; "C66"]

To a stirred mixture of tert-butyl 7-(6-bromo-8-fluoro-1-oxoisoquinolin-2(1H)-yl)-4-azaspiro[2.5]octane-4-carboxylate (80 mg, 0.173 mmol, 1 equiv) and bis(pinacolato)diboron (54 mg, 0.212 mmol, 1.2 equiv) in dioxane (5 mL) was added KOAc (35 mg, 0.354 mmol, 2 equiv) and Pd(dppf)Cl₂ (13 mg, 0.018 mmol, 0.1 equiv) in portions at room temperature under nitrogen atmosphere. The resulting mixture was stirred for 4 h at 100° C. under nitrogen atmosphere, then cooled to room temperature. To the resulting mixture was added 5-bromo-2,7-dimethylindazole (40 mg, 0.177 mmol, 1 equiv), K₃PO₄ (75.05 mg, 0.354 mmol, 2 equiv), H₂O (1 mL) and Pd(dppf)Cl₂ (13 mg, 0.018 mmol, 0.1 equiv) in portions at room temperature. The reaction mixture was stirred for an additional 4 h at 100° C., then cooled to room temperature. The resulting mixture was diluted with H₂O (15 mL) and extracted with ethyl acetate (3×5 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with EA to afford tert-butyl 7-[6-(2,7-dimethylindazol-5-yl)-8-fluoro-1-oxoisoquinolin-2-yl]-4-azaspiro[2.5]octane-4-carboxylate (30 mg, 33%) as an oil. LCMS (ES, m/z): 517 [M+H]⁺.

### Synthesis of Compound 415

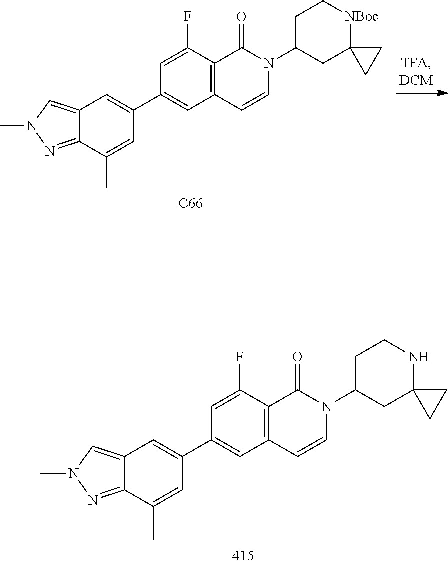 [SMILES: Cc1cc(-c2cc(F)c3c(=O)n(C4CCN(C(=O)OC(C)(C)C)C5(CC5)C4)ccc3c2)cc2cn(C)nc12 ; "C66" ; Cc1cc(-c2cc(F)c3c(=O)n(C4CCNC5(CC5)C4)ccc3c2)cc2cn(C)nc12 ; "415"]

A solution of tert-butyl 7-[6-(2,7-dimethylindazol-5-yl)-8-fluoro-1-oxoisoquinolin-2-yl]-4-azaspiro[2.5]octane-4-carboxylate (40 mg, 0.077 mmol, 1 equiv) in TFA (1 mL) and DCM (1 mL) was stirred for 2 h at room temperature. The resulting mixture was concentrated under reduced pressure to give a residue. The residue was basified to pH 8 with 7 M NH₃ in MeOH. The resulting mixture was concentrated under reduced pressure to give a residue. The residue was purified by reverse flash chromatography (Condition 2, Gradient 8) to afford 2-{4-azaspiro[2.5]octan-7-yl}-6-(2,7-dimethylindazol-5-yl)-8-fluoroisoquinolin-1-one (15 mg, 47%) as a solid. LCMS (ES, m/z): 417 [M+H]^{+ 1}H NMR (300 MHz, DMSO-d₆) δ 8.43 (s, 1H), 8.01 (d, J=1.7 Hz, 1H), 7.82 (d, J=1.7 Hz, 1H), 7.63-7.49 (m, 2H), 7.50 (s, 1H), 6.72 (dd, J=7.6, 2.1 Hz, 1H), 5.04 (d, J=9.1 Hz, 1H), 4.21 (s, 3H), 3.07-2.96 (m, 1H), 2.76-2.66 (m, 1H), 2.58 (s, 3H), 2.18 (t, J=12.0 Hz, 1H), 1.74 (q, J=7.5, 5.0 Hz, 2H), 1.13 (dd, J=11.9, 3.9 Hz, 1H), 0.57 (q, J=6.2 Hz, 1H), 0.45 (d, J=6.6 Hz, 1H), 0.38 (dt, J=12.2, 5.7 Hz, 3H).

### Example 116: Synthesis of Compound 416

### Synthesis of Intermediate C67

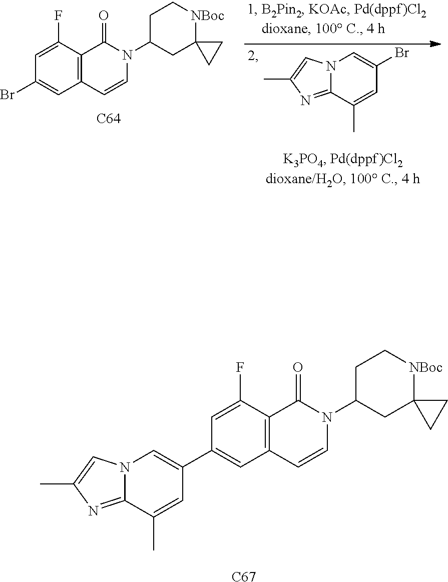 [SMILES: "C64" ; CC(C)(C)OC(=O)N1CCC(n2ccc3cc(Br)cc(F)c3c2=O)CC12CC2 ; Cc1cn2cc(Br)cc(C)c2n1 ; Cc1cn2cc(-c3cc(F)c4c(=O)n(C5CCN(C(=O)OC(C)(C)C)C6(CC6)C5)ccc4c3)cc(C)c2n1 ; "C67"]

To a stirred mixture of tert-butyl 7-(6-bromo-8-fluoro-1-oxoisoquinolin-2-yl)-4-azaspiro[2.5]octane-4-carboxylate (80 mg, 0.177 mmol, 1 equiv) and bis(pinacolato)diboron (54 mg, 0.212 mmol, 1.2 equiv) in dioxane (5 mL) was added KOAc (35 mg, 0.354 mmol, 2 equiv) and Pd(dppf)Cl₂ (13 mg, 0.018 mmol, 0.1 equiv) in portions at room temperature under nitrogen atmosphere. The resulting mixture was stirred for 4 h at 100° C. under nitrogen atmosphere, then cooled to room temperature. To the reaction mixture was added 6-bromo-2,8-dimethylimidazo[1,2-a]pyridine (40 mg, 0.177 mmol, 1 equiv), K₃PO₄ (75 mg, 0.354 mmol, 2 equiv), H₂O (0.4 mL), and Pd(dppf)Cl₂ (2 mg, 0.002 mmol, 0.1 equiv) in portions at room temperature. The resulting mixture was stirred for an additional 4 h at 100° C., then cooled to room temperature. The resulting mixture was diluted with H₂O (15 mL) and extracted with ethyl acetate (3×5 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with EA to afford tert-butyl 7-(6-{2,8-dimethylimidazo[1,2-a]pyridin-6-yl}-8-fluoro-1-oxoisoquinolin-2-yl)-4-azaspiro[2.5]octane-4-carboxylate (50 mg, 55%) as an oil. LCMS (ES, m/z): 517 [M+H]⁺.

### Synthesis of Compound 416

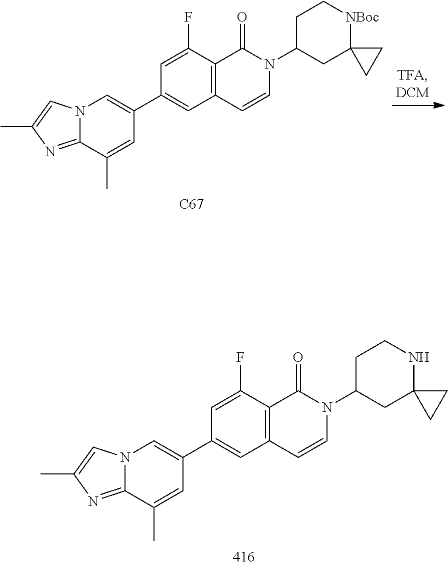 [SMILES: Cc1cn2cc(-c3cc(F)c4c(=O)n(C5CCN(C(=O)OC(C)(C)C)C6(CC6)C5)ccc4c3)cc(C)c2n1 ; "C67" ; Cc1cn2cc(-c3cc(F)c4c(=O)n(C5CCNC6(CC6)C5)ccc4c3)cc(C)c2n1 ; "416"]

A mixture of tert-butyl 7-(6-{2,8-dimethylimidazo[1,2-a]pyridin-6-yl}-8-fluoro-1-oxoisoquinolin-2-yl)-4-azaspiro[2.5]octane-4-carboxylate (40 mg, 0.077 mmol, 1 equiv) in TFA (2 mL) and DCM (2 mL) was stirred for 2 h at room temperature. The resulting mixture was concentrated under reduced pressure to give a residue. The residue was basified to pH 8 with 7 M NH₃ in methanol. The resulting mixture was concentrated under reduced pressure to give a residue. The residue was purified by reverse flash chromatography (Condition 2, Gradient 9) to afford 2-{4-azaspiro[2.5]octan-7-yl}-6-{2,8-dimethylimidazo[1,2-a]pyridin-6-yl}-8-fluoroisoquinolin-1-one (15 mg, 47%) as a solid. LCMS (ES, m/z): 417 [M+H]⁺. ¹H NMR (300 MHz, DMSO-d₆) δ 8.91 (d, J=1.9 Hz, 1H), 7.83 (d, J=1.7 Hz, 1H), 7.69 (s, 1H), 7.65-7.53 (m, 2H), 7.51 (s, 1H), 6.69 (dd, J=7.6, 2.1 Hz, 1H), 5.03 (dd, J=11.0, 6.5 Hz, 1H), 3.06-2.96 (m, 1H), 2.71 (q, J=12.5, 9.4 Hz, 1H), 2.53 (s, 3H), 2.37 (s, 3H), 2.18 (t, J=11.9 Hz, 1H), 1.72 (p, J=5.0, 4.0 Hz, 2H), 1.13 (dd, J=11.9, 3.9 Hz, 1H), 0.57 (q, J=6.3 Hz, 1H), 0.45 (d, J=6.7 Hz, 2H), 0.37 (q, J=6.5 Hz, 1H).

### Example 117: Synthesis of Compound 413

### Synthesis of Intermediate C68

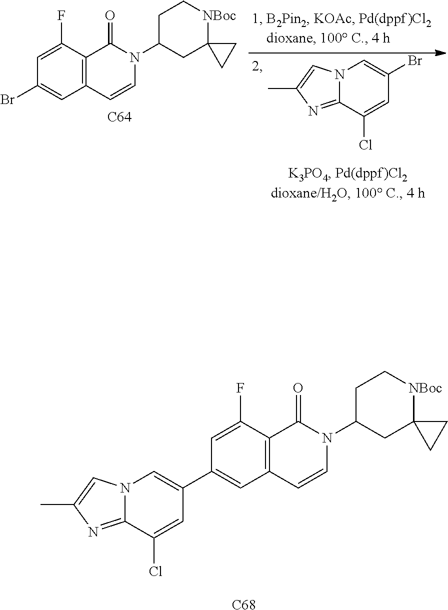 [SMILES: "C64" ; CC(C)(C)OC(=O)N1CCC(n2ccc3cc(Br)cc(F)c3c2=O)CC12CC2 ; Cc1cn2cc(Br)cc(Cl)c2n1 ; Cc1cn2cc(-c3cc(F)c4c(=O)n(C5CCN(C(=O)OC(C)(C)C)C6(CC6)C5)ccc4c3)cc(Cl)c2n1 ; "C68"]

To a stirred mixture of tert-butyl 7-(6-bromo-8-fluoro-1-oxoisoquinolin-2-yl)-4-azaspiro[2.5]octane-4-carboxylate (90 mg, 0.199 mmol, 1 equiv) and bis(pinacolato)diboron (61 mg, 0.239 mmol, 1.2 equiv) in dioxane (5 mL) was added KOAc (39 mg, 0.398 mmol, 2 equiv) and Pd(dppf)Cl₂ (15 mg, 0.020 mmol, 0.1 equiv) in portions at room temperature under nitrogen atmosphere. The resulting mixture was stirred for 4 h at 100° C. under nitrogen atmosphere, then cooled to room temperature. To the resulting mixture was added 6-bromo-8-chloro-2-methylimidazo[1,2-a]pyridine (49 mg, 0.199 mmol, 1 equiv), H₂O (1 mL), K₃PO₄ (85 mg, 0.398 mmol, 2 equiv), and Pd(dppf)Cl₂ (15 mg, 0.020 mmol, 0.1 equiv) in portions at room temperature. The resulting mixture was stirred for an additional 4 h at 100° C., then cooled to room temperature. The resulting mixture were diluted with H₂O (10 mL) and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 7-(6-{8-chloro-2-methylimidazo[1,2-a]pyridin-6-yl}-8-fluoro-1-oxoisoquinolin-2-yl)-4-azaspiro[2.5]octane-4-carboxylate (70 mg, 65%) as a solid. LCMS (ES, m/z): 537 [M+H]⁺.

### Synthesis of Compound 413

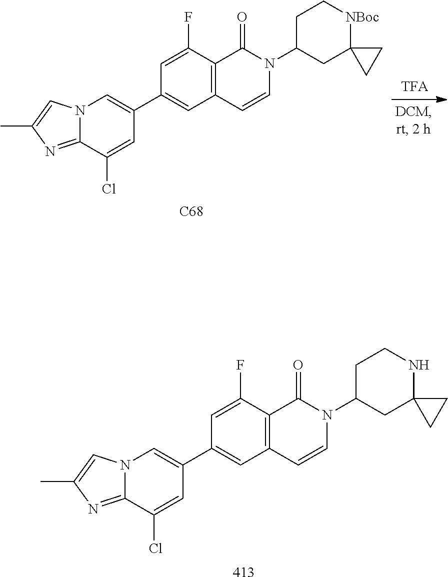 [SMILES: Cc1cn2cc(-c3cc(F)c4c(=O)n(C5CCN(C(=O)OC(C)(C)C)C6(CC6)C5)ccc4c3)cc(Cl)c2n1 ; "C68" ; Cc1cn2cc(-c3cc(F)c4c(=O)n(C5CCNC6(CC6)C5)ccc4c3)cc(Cl)c2n1 ; "413"]

A solution of tert-butyl 7-(6-{8-chloro-2-methylimidazo[1,2-a]pyridin-6-yl}-8-fluoro-1-oxoisoquinolin-2-yl)-4-azaspiro[2.5]octane-4-carboxylate (65 mg, 0.121 mmol, 1 equiv) in 2,2,2-trifluoroacetic acid (2 mL) and DCM (2 mL) was stirred for 2 h at room temperature. The resulting mixture was concentrated under reduced pressure to give a residue. The residue was basified to pH 8 with 7 M NH₃ in methanol. The resulting mixture was concentrated under vacuum to give a residue. The residue was purified by reverse flash chromatography (Condition 2, Gradient 4) to afford 2-{4-azaspiro[2.5]octan-7-yl}-6-{8-chloro-2-methylimidazo[1,2-a]pyridin-6-yl}-8-fluoroisoquinolin-1-one (30 mg, 57%) as a solid. LCMS (ES, m/z): 437 [M+H]⁺. ¹H NMR (300 MHz, DMSO-d₆) δ 9.10 (d, J=1.6 Hz, 1H), 7.90 (dd, J=7.7, 1.7 Hz, 2H), 7.84 (d, J=1.1 Hz, 1H), 7.65 (dd, J=13.4, 1.8 Hz, 1H), 7.57 (d, J=7.5 Hz, 1H), 6.69 (dd, J=7.6, 2.1 Hz, 1H), 5.04 (d, J=9.8 Hz, 1H), 3.01 (d, J=13.0 Hz, 1H), 2.79-2.63 (m, 1H), 2.40 (s, 3H), 2.18 (t, J=12.0 Hz, 1H), 1.73 (q, J=7.5, 6.9 Hz, 2H), 1.13 (dd, J=11.9, 3.9 Hz, 1H), 0.57 (q, J=6.3 Hz, 1H), 0.50-0.39 (m, 2H), 0.36 (dd, J=7.5, 5.0 Hz, 1H).

### Example 118: Synthesis of Compound 428

### Synthesis of Intermediate C69

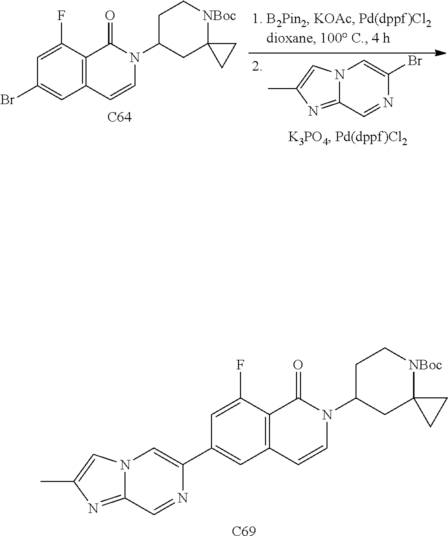 [SMILES: CC(C)(C)OC(=O)N1CCC(n2ccc3cc(Br)cc(F)c3c2=O)CC12CC2 ; Cc1cn2cc(Br)ncc2n1 ; "C64" ; Cc1cn2cc(-c3cc(F)c4c(=O)n(C5CCN(C(=O)OC(C)(C)C)C6(CC6)C5)ccc4c3)ncc2n1 ; "C69"]

To a stirred mixture of tert-butyl 7-(6-bromo-8-fluoro-1-oxoisoquinolin-2(1H)-yl)-4-azaspiro[2.5]octane-4-carboxylate (80 mg, 0.173 mmol, 1 equiv) and bis(pinacolato)diboron (54 mg, 0.212 mmol, 1.2 equiv) in dioxane (5 mL) was added KOAc (35 mg, 0.354 mmol, 2 equiv) and Pd(dppf)Cl₂ (13 mg, 0.018 mmol, 0.1 equiv) in portions at room temperature under nitrogen atmosphere. The resulting mixture was stirred for 4 h at 100° C. under nitrogen atmosphere, then cooled to room temperature. To the resulting mixture was added 6-bromo-2-methylimidazo[1,2-a]pyrazine (41 mg, 0.193 mmol, 1.1 equiv), K₃PO₄ (110 mg, 0.519 mmol, 3 equiv), Pd(dppf)Cl₂ (12 mg, 0.017 mmol, 0.1 equiv), and H₂O (1 mL) at room temperature under nitrogen atmosphere. The resulting mixture was stirred for an additional 4 h at 100° C., then cooled to room temperature. The reaction mixture was quenched with water (10 mL) at room temperature, then extracted with ethyl acetate (2×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with PE/EA (2:1) to afford tert-butyl 7-(8-fluoro-6-{2-methylimidazo[1,2-a]pyrazin-6-yl}-1-oxoisoquinolin-2-yl)-4-azaspiro[2.5]octane-4-carboxylate (50 mg, 56%) as a solid. LCMS (ES, m/z): 503 [M+H]⁺.

### Synthesis of Compound 428

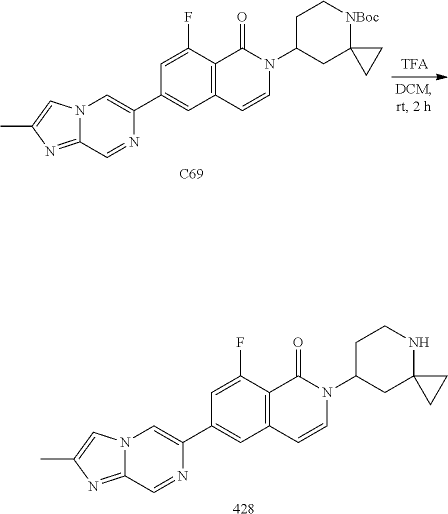 [SMILES: Cc1cn2cc(-c3cc(F)c4c(=O)n(C5CCN(C(=O)OC(C)(C)C)C6(CC6)C5)ccc4c3)ncc2n1 ; "C69" ; Cc1cn2cc(-c3cc(F)c4c(=O)n(C5CCNC6(CC6)C5)ccc4c3)ncc2n1 ; "428"]

A mixture of tert-butyl 7-(8-fluoro-6-{2-methylimidazo[1,2-a]pyrazin-6-yl}-1-oxoisoquinolin-2-yl)-4-azaspiro[2.5]octane-4-carboxylate (50 mg, 0.099 mmol, 1 equiv) and trifluoroacetic acid (5 mL) in DCM (5 mL) was stirred for 2 h at room temperature. The resulting mixture was concentrated under vacuum to give a residue. The residue was purified by Prep-HPLC (Condition 5, Gradient 1) to afford 8-fluoro-6-(2-methylimidazo[1,2-a]pyrazin-6-yl)-2-(4-azaspiro[2.5]octan-7-yl)isoquinolin-1(2H)-one (15 mg, 30%) as a solid. LCMS (ES, m/z): 404 [M+H]⁺. ¹H NMR (300 MHz, Methanol-d₄) δ 9.26 (d, J=1.5 Hz, 1H), 9.14 (s, 1H), 8.18 (s, 1H), 8.00 (s, 1H), 7.92 (d, J=13.1 Hz, 1H), 7.48 (d, J=7.6 Hz, 1H), 6.87 (d, J=6.0 Hz, 1H), 5.04 (d, J=9.1 Hz, 1H), 3.65 (d, J=12.6 Hz, 2H), 2.75 (t, J=13.0 Hz, 1H), 2.59 (d, J=0.9 Hz, 3H), 2.29 (s, 2H), 1.64 (d, J=13.1 Hz, 1H), 1.32 (s, 4H), 1.19-0.99 (m, 3H). ¹⁹F NMR (282 MHz, Methanol-d₄) δ−77.28, −111.39.

### Example 119: Synthesis of Compound 417

### Synthesis of Intermediate C70

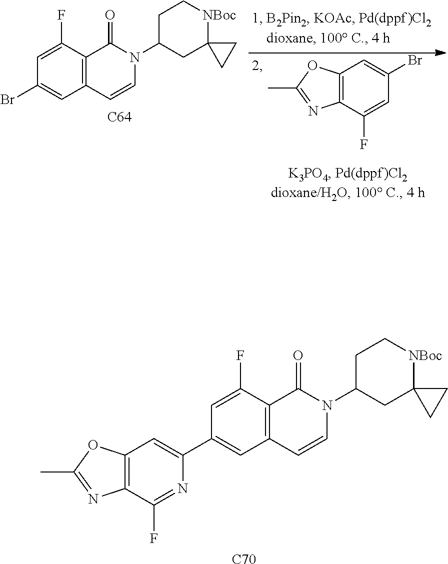 [SMILES: "C64" ; CC(C)(C)OC(=O)N1CCC(n2ccc3cc(Br)cc(F)c3c2=O)CC12CC2 ; Cc1nc2c(F)cc(Br)cc2o1 ; Cc1nc2c(F)nc(-c3cc(F)c4c(=O)n(C5CCN(C(=O)OC(C)(C)C)C6(CC6)C5)ccc4c3)cc2o1 ; "C70"]

To a stirred mixture of tert-butyl 7-(6-bromo-8-fluoro-1-oxoisoquinolin-2-yl)-4-azaspiro[2.5]octane-4-carboxylate (90 mg, 0.199 mmol, 1 equiv) and bis(pinacolato)diboron (61 mg, 0.239 mmol, 1.2 equiv) in dioxane (5 mL) was added KOAc (39 mg, 0.398 mmol, 2 equiv) and Pd(dppf)Cl₂ (15 mg, 0.020 mmol, 0.1 equiv) in portions at room temperature under nitrogen atmosphere. The resulting mixture was stirred for 4 h at 100° C. under nitrogen atmosphere, then cooled to room temperature. To the resulting mixture was added 6-bromo-4-fluoro-2-methyl-1,3-benzoxazole (46 mg, 0.199 mmol, 1 equiv), H₂O (1 mL), K₃PO₄ (85 mg, 0.398 mmol, 2 equiv), and Pd(dppf)Cl₂ (15 mg, 0.020 mmol, 0.1 equiv) in portions at room temperature. The resulting mixture was stirred for an additional 4 h at 100° C., then cooled to room temperature. The resulting mixture was concentrated under reduced pressure to give a residue. The residue was purified by silica gel column chromatography, eluted with PE/EA (1:10) to afford tert-butyl 7-[8-fluoro-6-(4-fluoro-2-methyl-1,3-benzoxazol-6-yl)-1-oxoisoquinolin-2-yl]-4-azaspiro[2.5]octane-4-carboxylate (45.8 mg, 44%) as an oil. LCMS (ES, m/z): 522 [M+H]⁺.

### Synthesis of Compound 417

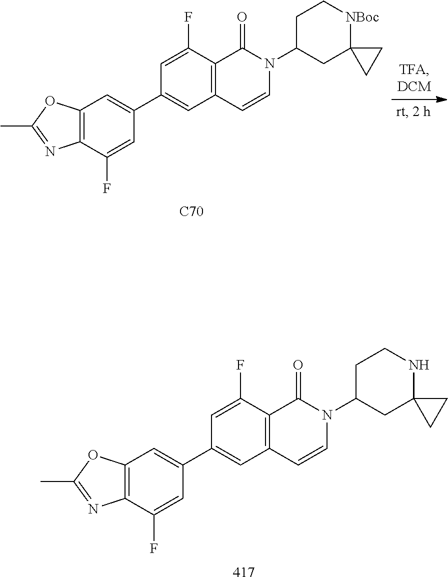 [SMILES: Cc1nc2c(F)cc(-c3cc(F)c4c(=O)n(C5CCN(C(=O)OC(C)(C)C)C6(CC6)C5)ccc4c3)cc2o1 ; "C70" ; Cc1nc2c(F)cc(-c3cc(F)c4c(=O)n(C5CCNC6(CC6)C5)ccc4c3)cc2o1 ; "417"]

A solution of tert-butyl 7-[8-fluoro-6-(4-fluoro-2-methyl-1,3-benzoxazol-6-yl)-1-oxoisoquinolin-2-yl]-4-azaspiro[2.5]octane-4-carboxylate (35 mg, 0.067 mmol, 1 equiv) in TFA (2 mL) and DCM (2 mL) was stirred for 2 h at room temperature. The resulting mixture was concentrated under reduced pressure to give a residue. The residue was basified to pH 8 with 7 M NH₃ in methanol. The resulting mixture was concentrated under reduced pressure to give a residue. The residue was purified by reverse flash chromatography (Condition 2, Gradient 10) to afford 2-{4-azaspiro[2.5]octan-7-yl}-8-fluoro-6-(4-fluoro-2-methyl-1,3-benzoxazol-6-yl)isoquinolin-1-one (13 mg, 46%) as a solid. LCMS (ES, m/z): 422 [M+H]⁺. ¹H NMR (300 MHz, DMSO-d₆) δ 8.09 (d, J=1.4 Hz, 1H), 7.93 (d, J=1.8 Hz, 1H), 7.73 (ddd, J=23.2, 12.4, 1.6 Hz, 2H), 7.57 (d, J=7.6 Hz, 1H), 6.70 (dd, J=7.6, 2.1 Hz, 1H), 5.03 (d, J=8.5 Hz, 1H), 3.07-2.96 (m, 1H), 2.71 (dd, J=16.3, 11.7 Hz, 1H), 2.69 (s, 3H), 2.18 (t, J=12.0 Hz, 1H), 1.73 (q, J=6.0, 5.4 Hz, 2H), 1.13 (dd, J=11.9, 4.0 Hz, 1H), 0.58 (dd, J=7.3, 4.8 Hz, 1H), 0.41 (dd, J=23.5, 6.1 Hz, 3H).

### Example 120: Synthesis of Compound 418

### Synthesis of Intermediate C71

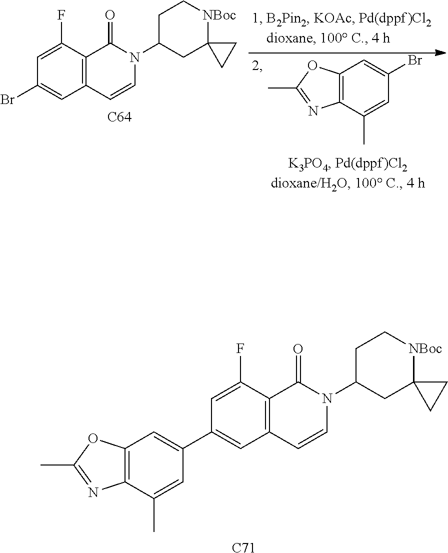 [SMILES: "C64" ; CC(C)(C)OC(=O)N1CCC(n2ccc3cc(Br)cc(F)c3c2=O)CC12CC2 ; Cc1nc2c(C)cc(Br)cc2o1 ; Cc1nc2c(C)cc(-c3cc(F)c4c(=O)n(C5CCN(C(=O)OC(C)(C)C)C6(CC6)C5)ccc4c3)cc2o1 ; "C71"]

To a stirred mixture of tert-butyl 7-(6-bromo-8-fluoro-1-oxoisoquinolin-2-yl)-4-azaspiro[2.5]octane-4-carboxylate (100 mg, 0.222 mmol, 1 equiv) and bis(pinacolato)diboron (68 mg, 0.266 mmol, 1.2 equiv) in dioxane (5 mL) was added KOAc (43.49 mg, 0.444 mmol, 2 equiv) and Pd(dppf)Cl₂ (17 mg, 0.022 mmol, 0.1 equiv) in portions at room temperature under nitrogen atmosphere. The resulting mixture was stirred for 4 h at 100° C. under nitrogen atmosphere, then cooled to room temperature. To the resulting mixture was added 6-bromo-2,4-dimethyl-1,3-benzoxazole (50 mg, 0.222 mmol, 1 equiv), K₃PO₄ (94 mg, 0.444 mmol, 2 equiv), Pd(dppf)Cl₂ (17 mg, 0.022 mmol, 0.1 equiv), and H₂O (1 mL) in portions at room temperature. The resulting mixture was stirred for an additional 4 h at 100° C., then cooled to room temperature. The resulting mixture was concentrated under reduced pressure to give a residue. The residue was purified by silica gel column chromatography, eluted with PE/EA (1:10) to afford tert-butyl 7-[6-(2,4-dimethyl-1,3-benzoxazol-6-yl)-8-fluoro-1-oxoisoquinolin-2-yl]-4-azaspiro[2.5]octane-4-carboxylate (40 mg, 35%) as an oil. LCMS (ES, m/z): 518 [M+H]⁺.

### Synthesis of Compound 418

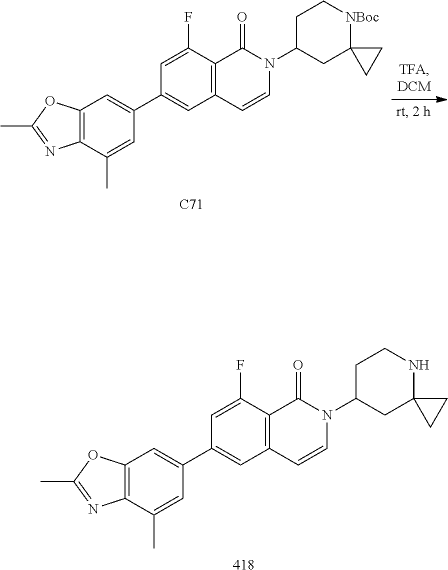 [SMILES: Cc1nc2c(C)cc(-c3cc(F)c4c(=O)n(C5CCN(C(=O)OC(C)(C)C)C6(CC6)C5)ccc4c3)cc2o1 ; "C71" ; Cc1nc2c(C)cc(-c3cc(F)c4c(=O)n(C5CCNC6(CC6)C5)ccc4c3)cc2o1 ; "418"]

A solution of tert-butyl 7-[6-(2,4-dimethyl-1,3-benzoxazol-6-yl)-8-fluoro-1-oxoisoquinolin-2-yl]-4-azaspiro[2.5]octane-4-carboxylate (40 mg, 0.077 mmol, 1 equiv) in TFA (2 mL) and DCM (2 mL) was stirred for 2 h at room temperature. The resulting mixture was concentrated under reduced pressure to give a residue. The residue was basified to pH 8 with 7 M NH₃ in methanol. The resulting mixture was concentrated under reduced pressure to give a residue. The residue was purified by reverse flash chromatography (Condition 2, Gradient 9) to afford 2-{4-azaspiro[2.5]octan-7-yl}-6-(2,4-dimethyl-1,3-benzoxazol-6-yl)-8-fluoroisoquinolin-1-one (15 mg, 46%) as a solid. LCMS (ES, m/z): 418 [M+H]⁺. ¹H NMR (300 MHz, DMSO-d₆) δ 7.96 (d, J=1.7 Hz, 1H), 7.86 (d, J=1.8 Hz, 1H), 7.70-7.58 (m, 2H), 7.56 (d, J=7.5 Hz, 1H), 6.72 (dd, J=7.6, 2.1 Hz, 1H), 5.04 (d, J=9.7 Hz, 1H), 3.01 (d, J=12.5 Hz, 1H), 2.79-2.67 (m, 1H), 2.65 (s, 3H), 2.58 (s, 3H), 2.18 (t, J=12.0 Hz, 1H), 1.74 (s, 2H), 1.13 (dd, J=12.0, 3.9 Hz, 1H), 0.57 (q, J=6.3 Hz, 1H), 0.41 (dd, J=23.5, 6.1 Hz, 3H).

### Example 121: Synthesis of Compound 429

### Synthesis of Intermediate C72

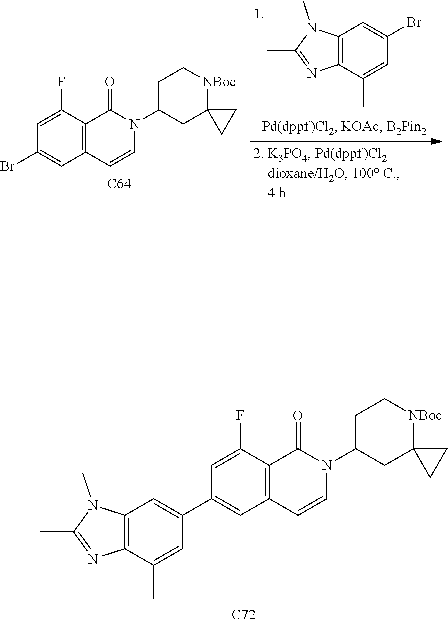 [SMILES: CC(C)(C)OC(=O)N1CCC(n2ccc3cc(Br)cc(F)c3c2=O)CC12CC2 ; Cc1cc(Br)cc2c1nc(C)n2C ; "C64" ; Cc1cc(-c2cc(F)c3c(=O)n(C4CCN(C(=O)OC(C)(C)C)C5(CC5)C4)ccc3c2)cc2c1nc(C)n2C ; "C72"]

To a stirred mixture of 6-bromo-1,2,4-trimethyl-1H-benzo[d]imidazole (80 mg, 0.333 mmol, 1.5 equiv) and B₂Pin₂ (113 mg, 0.444 mmol, 2.0 equiv) in 1,4-dioxane (8 mL) was added KOAc (66 mg, 0.666 mmol, 3.0 equiv) and Pd(dppf)Cl₂ (18 mg, 0.022 mmol, 0.1 equiv) at room temperature under nitrogen atmosphere. The reaction mixture was stirred for 3 h at 90° C. under nitrogen atmosphere, then cooled to room temperature. To the resulting mixture was added tert-butyl 7-(6-bromo-8-fluoro-1-oxoisoquinolin-2(1H)-yl)-4-azaspiro[2.5]octane-4-carboxylate (100 mg, 0.222 mmol, 1.0 equiv), K₃PO₄ (142 mg, 0.666 mmol, 3 equiv), H₂O (2 mL), and Pd(dppf)Cl₂ (18 mg, 0.022 mmol, 0.1 equiv) in portions at room temperature under nitrogen atmosphere. The resulting mixture was stirred for an additional 4 h at 100° C. The reaction mixture was quenched with water (20 mL), then extracted with ethyl acetate (3×20 mL). The organic layers were combined, washed with brine (2×20 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 7-[8-fluoro-1-oxo-6-(2,3,7-trimethyl-1,3-benzodiazol-5-yl)isoquinolin-2-yl]-4-azaspiro[2.5]octane-4-carboxylate (30 mg, 35%) as a solid. LCMS (ES, m/z):531 [M+H]⁺.

### Synthesis of Compound 429

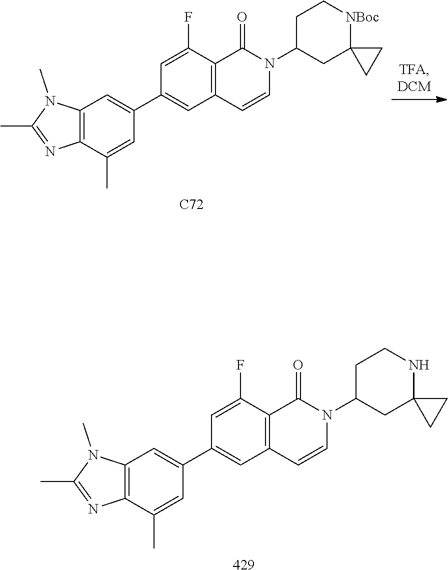 [SMILES: Cc1cc(-c2cc(F)c3c(=O)n(C4CCN(C(=O)OC(C)(C)C)C5(CC5)C4)ccc3c2)cc2c1nc(C)n2C ; "C72" ; Cc1cc(-c2cc(F)c3c(=O)n(C4CCNC5(CC5)C4)ccc3c2)cc2c1nc(C)n2C ; "429"]

A mixture of tert-butyl 7-[8-fluoro-1-oxo-6-(2,3,7-trimethyl-1,3-benzodiazol-5-yl)isoquinolin-2-yl]-4-azaspiro[2.5]octane-4-carboxylate (30 mg, 0.057 mmol, 1.00 equiv) and trifluoroacetic acid (32 mg, 0.285 mmol, 5 equiv) in DCM (2 mL) was stirred for 2 h at room temperature, then concentrated under reduced pressure to give a residue. The residue was neutralized to pH 8 with ammonia. The resulting mixture was concentrated under reduced pressure to give a residue. The residue was purified by reverse flash chromatography (Condition 2, Gradient 11) to afford 2-{4-azaspiro[2.5]octan-7-yl}-8-fluoro-6-{2-methylimidazo[1,2-a]pyrazin-6-yl}isoquinolin-1-one (10 mg, 37%) as a solid. LCMS (ES, m/z): 431[M+H]⁺. ¹H NMR (300 MHz, DMSO-d6) δ 7.89-7.79 (m, 2H), 7.70-7.60 (m, 1H), 7.54 (d, J=7.5 Hz, 1H), 7.47 (s, 1H), 6.73 (d, J=7.5 Hz, 1H), 5.05 (s, 1H), 3.80 (s, 3H), 3.02 (d, J=12.9 Hz, 1H), 2.57 (d, J=2.0 Hz, 6H), 2.19 (t, J=12.0 Hz, 1H), 1.74 (s, 2H), 1.18-1.08 (m, 1H), 0.64-0.54 (m, 1H), 0.46 (d, J=7.1 Hz, 2H), 0.38 (d, J=6.1 Hz, 1H). ¹⁹F NMR (282 MHz, DMSO) δ-110.75.

### Example 122: Synthesis of Compound 430

### Synthesis of Intermediate C73

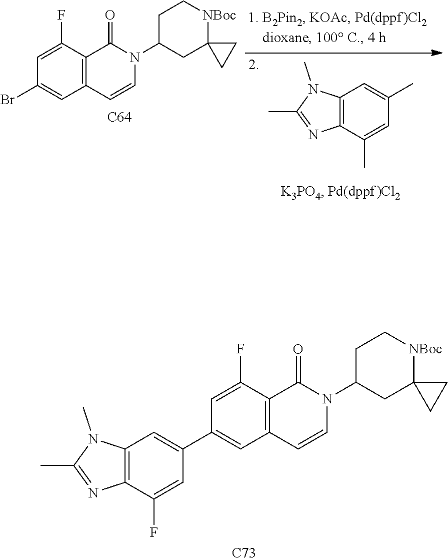

To a stirred mixture of tert-butyl 7-(6-bromo-8-fluoro-1-oxoisoquinolin-2(1H)-yl)-4-azaspiro[2.5]octane-4-carboxylate (80 mg, 0.173 mmol, 1 equiv) and bis(pinacolato)diboron (54 mg, 0.212 mmol, 1.2 equiv) in dioxane (5 mL) was added KOAc (35 mg, 0.354 mmol, 2 equiv) and Pd(dppf)Cl₂ (13 mg, 0.018 mmol, 0.1 equiv) in portions at room temperature under nitrogen atmosphere. The resulting mixture was stirred for 4 h at 100° C. under nitrogen atmosphere, then cooled to room temperature. To the resulting mixture was added 6-bromo-4-fluoro-1,2-dimethyl-1H-benzo[d]imidazole (47 mg, 0.193 mmol, 1.1 equiv), K₃PO₄ (110 mg, 0.519 mmol, 3 equiv), Pd(dppf)Cl₂ (12 mg, 0.017 mmol, 0.1 equiv), and H₂O (1 mL) at room temperature under nitrogen atmosphere. The resulting mixture was stirred for an additional 4 h at 100° C., then cooled to room temperature. The reaction mixture was quenched with water (10 mL) and extracted with ethyl acetate (2×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with PE/EA (2:1) to afford tert-butyl 7-[8-fluoro-6-(7-fluoro-2,3-dimethyl-1,3-benzodiazol-5-yl)-1-oxoisoquinolin-2-yl]-4-azaspiro[2.5]octane-4-carboxylate (60 mg, 63%) as a solid. LCMS (ES, m/z): 535 [M+H]⁺.

### Synthesis of Compound 430

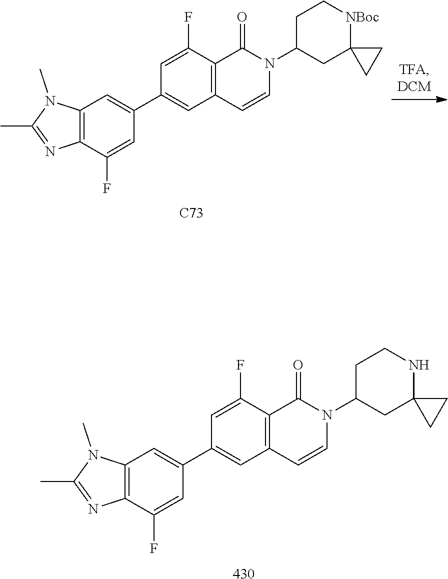 [SMILES: Cc1nc2c(F)cc(-c3cc(F)c4c(=O)n(C5CCN(C(=O)OC(C)(C)C)C6(CC6)C5)ccc4c3)cc2n1C ; "C73" ; Cc1nc2c(F)cc(-c3cc(F)c4c(=O)n(C5CCNC6(CC6)C5)ccc4c3)cc2n1C ; "430"]

A mixture of tert-butyl 7-[8-fluoro-6-(7-fluoro-2,3-dimethyl-1,3-benzodiazol-5-yl)-1-oxoisoquinolin-2-yl]-4-azaspiro[2.5]octane-4-carboxylate (30 mg, 0.056 mmol, 1 equiv) and TFA (2 mL) in DCM (5 mL) was stirred for 2 h at room temperature. The resulting mixture was concentrated under reduced pressure to give a residue. The residue was purified by reverse flash chromatography (Condition 2, Gradient 11) to afford 8-fluoro-6-(4-fluoro-1,2-dimethyl-1H-benzo[d]imidazol-6-yl)-2-(4-azaspiro[2.5]octan-7-yl)isoquinolin-1(2H)-one (8.2 mg, 29%) as a solid. LCMS (ES, m/z): 435 [M+H]⁺. ¹H NMR (300 MHz, DMSO-d₆) δ 7.93 (d, J=1.6 Hz, 2H), 7.77-7.66 (m, 1H), 7.60-7.47 (m, 2H), 6.72 (d, J=6.4 Hz, 1H), 5.04 (s, 1H), 3.84 (s, 3H), 3.03 (d, J=12.5 Hz, 1H), 2.63 (s, 1H), 2.58 (s, 3H), 2.20 (t, J=12.1 Hz, 2H), 1.75 (s, 2H), 1.14 (dd, J=11.7, 4.0 Hz, 1H), 0.59 (d, J=6.2 Hz, 1H), 0.43 (dd, J=23.8, 6.5 Hz, 2H). ¹⁹F NMR (282 MHz, DMSO-d₆) δ−110.53, −129.33.

### Example 123: Synthesis of Compound 414

### Synthesis of Intermediate C74

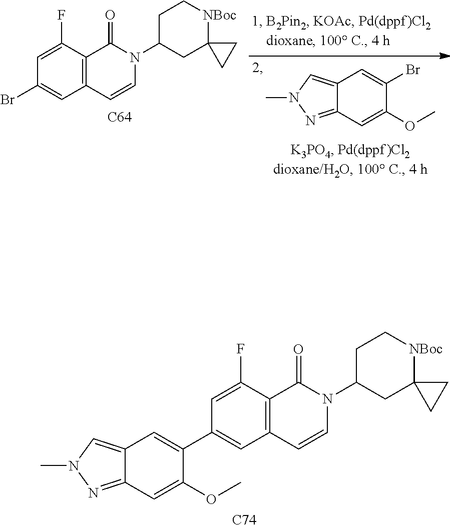

To a stirred mixture of tert-butyl 7-(6-bromo-8-fluoro-1-oxoisoquinolin-2-yl)-4-azaspiro[2.5]octane-4-carboxylate (100 mg, 0.222 mmol, 1 equiv) and bis(pinacolato)diboron (68 mg, 0.266 mmol, 1.2 equiv) in dioxane (5 mL) was added KOAc (44 mg, 0.444 mmol, 2 equiv) and Pd(dppf)Cl₂ (16.21 mg, 0.022 mmol, 0.1 equiv) in portions at room temperature under nitrogen atmosphere. The resulting mixture was stirred for 4 h at 100° C. under nitrogen atmosphere, then cooled to room temperature. To the resulting mixture was added 5-bromo-6-methoxy-2-methylindazole (54 mg, 0.222 mmol, 1 equiv), H₂O (1 mL), K₃PO₄ (94 mg, 0.444 mmol, 2 equiv), and Pd(dppf)Cl₂ (16 mg, 0.022 mmol, 0.1 equiv) in portions at room temperature. The resulting mixture was stirred for an additional 4 h at 100° C., then cooled to room temperature. The resulting mixture was diluted with H₂O (10 mL) and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 7-[8-fluoro-6-(6-methoxy-2-methylindazol-5-yl)-1-oxoisoquinolin-2-yl]-4-azaspiro[2.5]octane-4-carboxylate (50 mg, 42%) as a solid. LCMS (ES, m/z): 533 [M+H]⁺.

### Synthesis of Compound 414

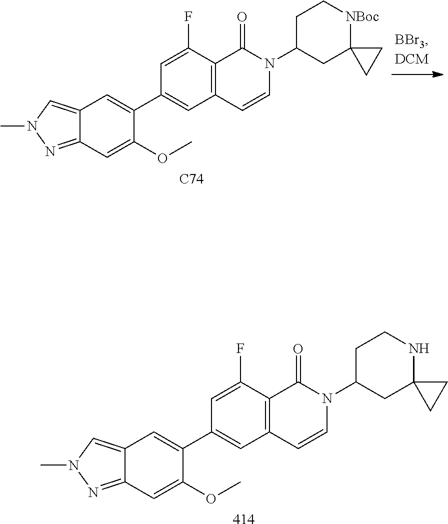

To a stirred solution of tert-butyl 7-[8-fluoro-6-(6-methoxy-2-methylindazol-5-yl)-1-oxoisoquinolin-2-yl]-4-azaspiro[2.5]octane-4-carboxylate (5 mg, 0.009 mmol, 1 equiv) in DCM (0.4 mL) was added BBr₃ (12 mg, 0.045 mmol, 5 equiv) dropwise at 0° C. The resulting mixture was stirred for 2 h at room temperature, then concentrated under reduced pressure. The reaction mixture was quenched with methanol at room temperature. The resulting mixture was purified by reverse flash chromatography (Condition 2, Gradient 9) to afford 2-{4-azaspiro[2.5]octan-7-yl}-8-fluoro-6-(6-hydroxy-2-methylindazol-5-yl)isoquinolin-1-one (12.3 mg) as a solid. LCMS (ES, m/z): 419 [M+H]⁺. ¹H NMR (300 MHz, DMSO-d₆) δ 9.79 (s, 1H), 8.26 (s, 1H), 7.72 (s, 1H), 7.61 (d, J=1.6 Hz, 1H), 7.51 (d, J=7.6 Hz, 1H), 7.39 (dd, J=13.5, 1.6 Hz, 1H), 6.95 (s, 1H), 6.69 (dd, J=7.7, 2.1 Hz, 1H), 5.10-5.01 (m, 1H), 4.09 (s, 3H), 3.01 (d, J=12.7 Hz, OH), 2.69 (d, J=12.7 Hz, 1H), 2.19 (t, J=12.0 Hz, 1H), 1.73 (s, 2H), 1.13 (dd, J=11.8, 4.0 Hz, 2H), 0.57 (q, J=6.2 Hz, 1H), 0.45 (d, J=6.2 Hz, 1H), 0.37 (t, J=6.0 Hz, 3H).

### Example 124: Synthesis of Compound 419

### Synthesis of Intermediate C75

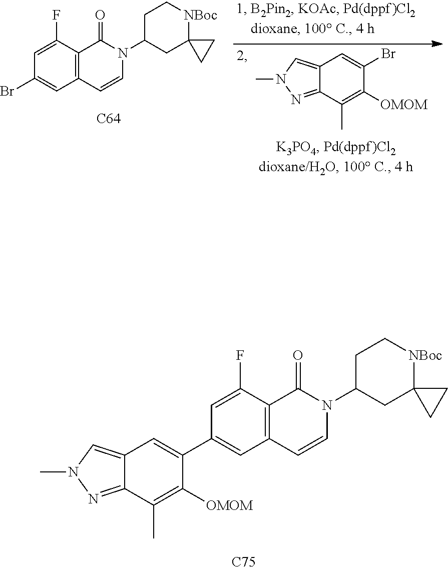

To a stirred mixture of tert-butyl 7-(6-bromo-8-fluoro-1-oxoisoquinolin-2-yl)-4-azaspiro[2.5]octane-4-carboxylate (100 mg, 0.222 mmol, 1 equiv) and bis(pinacolato)diboron (68 mg, 0.266 mmol, 1.2 equiv) in dioxane (5 mL) was added KOAc (44 mg, 0.444 mmol, 2 equiv) and Pd(dppf)Cl₂ (16 mg, 0.022 mmol, 0.1 equiv) in portions at room temperature under nitrogen atmosphere. The resulting mixture was stirred for 4 h at 100° C. under nitrogen atmosphere, then cooled to room temperature. To the resulting mixture was added 5-bromo-6-(methoxymethoxy)-2,7-dimethyl-2H-indazole (63 mg, 0.222 mmol, 1 equiv), Pd(dppf)Cl₂ (16 mg, 0.022 mmol, 0.1 equiv), H₂O (1 mL), and K₃PO₄ (94 mg, 0.444 mmol, 2 equiv) in portions at room temperature. The resulting mixture was stirred for additional 4 h at 100° C. under nitrogen atmosphere, then cooled to room temperature. The resulting mixture was concentrated under reduced pressure to give a residue. The residue was purified by silica gel column chromatography, eluted with PE/EA (1:10) to afford tert-butyl 7-{8-fluoro-6-[6-(methoxymethoxy)-2,7-dimethylindazol-5-yl]-1-oxoisoquinolin-2-yl}-4-azaspiro[2.5]octane-4-carboxylate (45 mg, 35%) as an oil. LCMS (ES, m/z): 577 [M+H]⁺.

### Synthesis of Compound 419

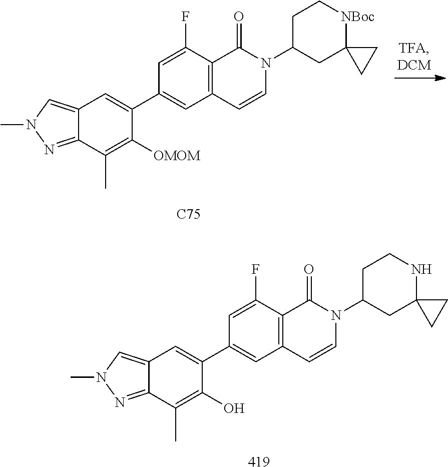

A solution of tert-butyl 7-{8-fluoro-6-[6-(methoxymethoxy)-2,7-dimethylindazol-5-yl]-1-oxoisoquinolin-2-yl}-4-azaspiro[2.5]octane-4-carboxylate (40 mg, 0.069 mmol, 1 equiv) in TFA (2 mL) and DCM (2 mL) was stirred for 2 h at room temperature. The resulting mixture was concentrated under reduced pressure to give a residue. The residue was basified to pH 8 with 7 M NH₃ in methanol. The resulting mixture was concentrated under vacuum to give a residue. The residue was purified by reverse flash chromatography (Condition 2, Gradient 9) to afford 2-{4-azaspiro[2.5]octan-7-yl}-8-fluoro-6-(6-hydroxy-2,7-dimethylindazol-5-yl)isoquinolin-1-one (16 mg, 53%) as a solid. LCMS (ES, m/z): 433 [M+H]⁺. ¹H NMR (300 MHz, DMSO-d₆) δ 8.46 (s, 1H), 8.25 (s, 1H), 7.58 (d, J=1.6 Hz, 1H), 7.51 (d, J=7.8 Hz, 2H), 7.35 (dd, J=13.4, 1.6 Hz, 1H), 6.69 (dd, J=7.6, 2.1 Hz, 1H), 5.04 (s, 1H), 4.13 (s, 3H), 3.01 (d, J=12.8 Hz, 1H), 2.71 (s, 1H), 2.41 (s, 3H), 2.19 (t, J=12.0 Hz, 1H), 1.74 (dd, J=9.3, 6.0 Hz, 2H), 1.13 (dd, J=12.1, 4.0 Hz, 1H), 0.57 (q, J=6.1 Hz, 1H), 0.42 (dd, J=22.5, 6.0 Hz, 3H).

### Example 125: Synthesis of Compound 420

### Synthesis of Intermediate C76

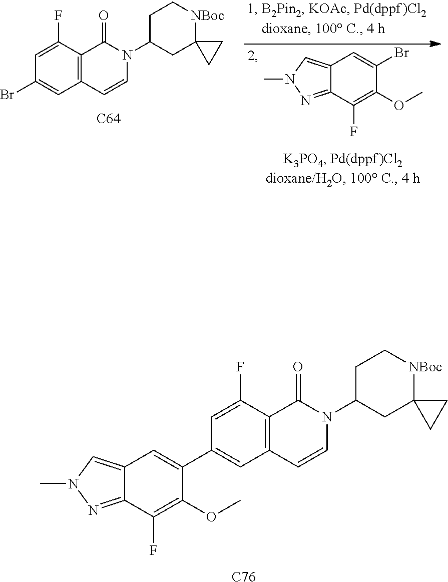

To a stirred mixture of tert-butyl 7-(6-bromo-8-fluoro-1-oxoisoquinolin-2-yl)-4-azaspiro[2.5]octane-4-carboxylate (100 mg, 0.222 mmol, 1 equiv) and bis(pinacolato)diboron (68 mg, 0.266 mmol, 1.2 equiv) in dioxane (5 mL) was added KOAc (44 mg, 0.444 mmol, 2 equiv) and Pd(dppf)Cl₂ (16 mg, 0.022 mmol, 0.1 equiv) in portions at room temperature under nitrogen atmosphere. The resulting mixture was stirred for 3 h at 100° C. under nitrogen atmosphere, then cooled to room temperature. To the resulting mixture was added 5-bromo-7-fluoro-6-methoxy-2-methylindazole (58 mg, 0.222 mmol, 1 equiv), H₂O (1 mL), K₃PO₄ (94 mg, 0.444 mmol, 2 equiv), and Pd(dppf)Cl₂ (16 mg, 0.022 mmol, 0.1 equiv) in portions at room temperature. The resulting mixture was stirred for additional 3 h at 100° C., then cooled to room temperature. The resulting mixture was diluted with H₂O (10 mL) and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 7-[8-fluoro-6-(7-fluoro-6-methoxy-2-methylindazol-5-yl)-1-oxoisoquinolin-2-yl]-4-azaspiro[2.5]octane-4-carboxylate (60 mg, 49%) as a solid. LCMS (ES, m/z): 551 [M+H]⁺.

### Synthesis of Compound 420

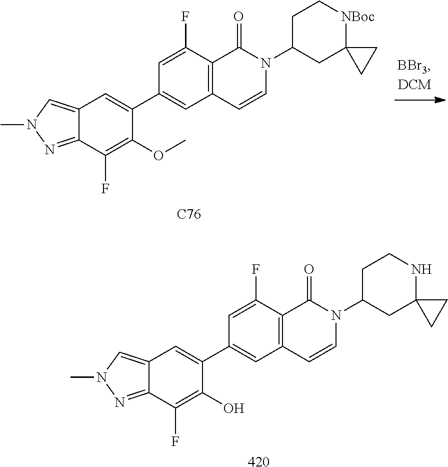 [SMILES: COc1c(-c2cc(F)c3c(=O)n(C4CCN(C(=O)OC(C)(C)C)C5(CC5)C4)ccc3c2)cc2cn(C)nc2c1F ; "C76" ; Cn1cc2cc(-c3cc(F)c4c(=O)n(C5CCNC6(CC6)C5)ccc4c3)c(O)c(F)c2n1 ; "420"]

To a stirred solution of tert-butyl 7-[8-fluoro-6-(7-fluoro-6-methoxy-2-methylindazol-5-yl)-1-oxoisoquinolin-2-yl]-4-azaspiro[2.5]octane-4-carboxylate (50 mg, 0.009 mmol, 1 equiv) in DCM (5 mL) was added BBr₃ (125 mg, 0.500 mmol, 5 equiv) dropwise at 0° C. under nitrogen atmosphere. The resulting mixture was stirred for 2 h at room temperature under nitrogen atmosphere, then concentrated under vacuum to give a residue. The residue was dissolved in water (2 mL). The resulting mixture was concentrated under reduced pressure to give a residue. The residue was purified by reverse flash chromatography (Condition 2, Gradient 8) to afford 2-{4-azaspiro[2.5]octan-7-yl}-8-fluoro-6-(7-fluoro-6-hydroxy-2-methylindazol-5-yl)isoquinolin-1-one (20 mg, 46%) as a solid. LCMS (ES, m/z): 437 [M+H]⁺. ¹H NMR (300 MHz, DMSO-d₆) δ 9.78 (s, 1H), 8.41 (d, J=2.7 Hz, 1H), 7.66-7.56 (m, 2H), 7.53 (d, J=7.6 Hz, 1H), 7.39 (dd, J=13.4, 1.6 Hz, 1H), 6.69 (dd, J=7.6, 2.1 Hz, 1H), 5.05 (dd, J=7.6, 2.1 Hz, 1H), 4.15 (s, 3H), 3.01 (d, J=12.5 Hz, 1H), 2.71 (s, 1H), 2.19 (t, J=12.0 Hz, 1H), 1.74 (dd, J=9.3, 5.8 Hz, 2H), 1.13 (dd, J=11.8, 3.9 Hz, 1H), 0.57 (q, J=6.1 Hz, 1H), 0.41 (dd, J=22.2, 5.9 Hz, 3H).

### Example 126: Synthesis of Compound 421

### Synthesis of Intermediate C77

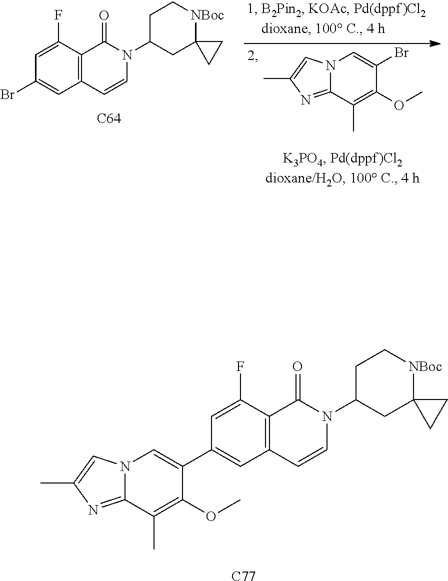

To a stirred mixture of tert-butyl 7-(6-bromo-8-fluoro-1-oxoisoquinolin-2-yl)-4-azaspiro[2.5]octane-4-carboxylate (100 mg, 0.222 mmol, 1 equiv) and bis(pinacolato)diboron (68 mg, 0.266 mmol, 1.2 equiv) in dioxane (5 mL) was added KOAc (44 mg, 0.444 mmol, 2 equiv) and Pd(dppf)Cl₂ (161 mg, 0.022 mmol, 0.1 equiv) in portions at room temperature under nitrogen atmosphere. The resulting mixture was stirred for 4 h at 100° C. under nitrogen atmosphere, then cooled to room temperature. To the resulting mixture was added 6-bromo-7-methoxy-2,8-dimethylimidazo[1,2-a]pyridine (56.52 mg, 0.222 mmol, 1 equiv), H₂O (1 mL), K₃PO₄ (94 mg, 0.444 mmol, 2 equiv), and Pd(dppf)Cl₂ (16 mg, 0.022 mmol, 0.1 equiv) in portions at room temperature. The resulting mixture was stirred for an additional 4 h at 100° C., then cooled to room temperature. The resulting mixture was diluted with H₂O (10 mL) and extracted with ethyl acetate (3×5 mL). The organic layers were combined, washed with brine (1×5 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 7-(8-fluoro-6-{7-methoxy-2,8-dimethylimidazo[1,2-a]pyridin-6-yl}-1-oxoisoquinolin-2-yl)-4-azaspiro[2.5]octane-4-carboxylate (50 mg, 41%) as a solid. LCMS (ES, m/z): 547 [M+H]⁺.

### Synthesis of Compound 421

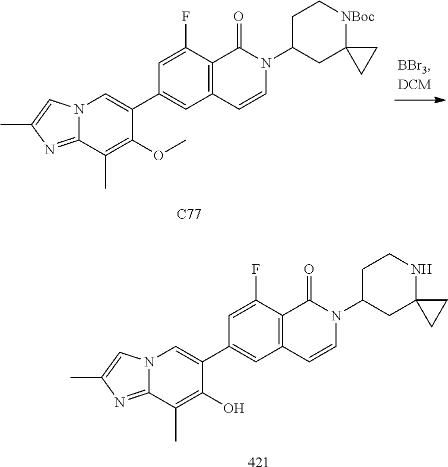 [SMILES: COc1c(-c2cc(F)c3c(=O)n(C4CCN(C(=O)OC(C)(C)C)C5(CC5)C4)ccc3c2)cn2cc(C)nc2c1C ; "C77" ; Cc1cn2cc(-c3cc(F)c4c(=O)n(C5CCNC6(CC6)C5)ccc4c3)c(O)c(C)c2n1 ; "421"]

To a stirred solution of tert-butyl 7-(8-fluoro-6-{7-methoxy-2,8-dimethylimidazo[1,2-a]pyridin-6-yl}-1-oxoisoquinolin-2-yl)-4-azaspiro[2.5]octane- 4-carboxylate (45 mg, 0.101 mmol, 1 equiv) in DCM (5 mL) was added BBr₃ (126 mg, 0.505 mmol, 5 equiv) dropwise at 0° C. under nitrogen atmosphere. The resulting mixture was stirred for 2 h at room temperature under nitrogen atmosphere, then concentrated under reduced pressure to give a residue. The residue was dissolved in water (5 mL). The resulting mixture was concentrated under vacuum to give a residue. The residue was purified by reverse flash chromatography (Condition 2, Gradient 8) to afford 2-{4-azaspiro[2.5]octan-7-yl}-8-fluoro-6-{7-hydroxy-2,8-dimethylimidazo[1,2-a]pyridin-6-yl}isoquinolin-1-one (14 mg, 39%) as a solid. LCMS (ES, m/z): 433 [M+H]⁺. ¹H NMR (300 MHz, DMSO-d₆) δ 8.33 (s, 1H), 7.73 (s, 1H), 7.58 (s, 1H), 7.49 (d, J=7.6 Hz, 1H), 7.26 (s, 1H), 6.65 (dd, J=7.7, 2.0 Hz, 1H), 5.08-4.97 (m, 1H), 2.99 (d, J=12.7 Hz, 1H), 2.68 (s, 1H), 2.29-2.19 (m, 3H), 2.17 (s, 2H), 2.13 (s, 3H), 1.83-1.67 (m, 2H), 1.11 (dd, J=12.1, 3.9 Hz, 1H), 0.57 (t, J=6.2 Hz, 1H), 0.49-0.30 (m, 2H).

### Example 127: Synthesis of Compound 431

### Synthesis of Intermediate C78

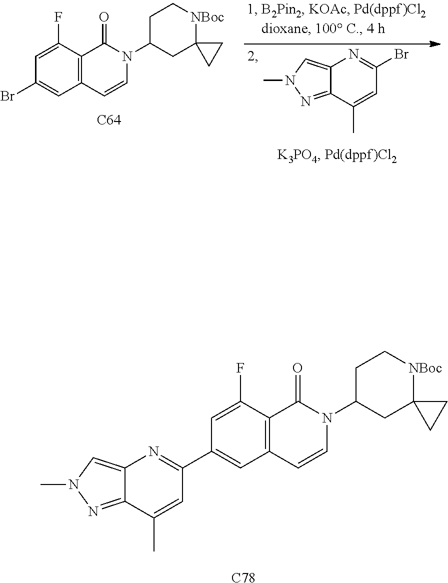 [SMILES: "C64" ; CC(C)(C)OC(=O)N1CCC(n2ccc3cc(Br)cc(F)c3c2=O)CC12CC2 ; Cc1cc(Br)nc2cn(C)nc12 ; Cc1cc(-c2cc(F)c3c(=O)n(C4CCN(C(=O)OC(C)(C)C)C5(CC5)C4)ccc3c2)nc2cn(C)nc12 ; "C78"]

To a stirred mixture of tert-butyl 7-(6-bromo-8-fluoro-1-oxoisoquinolin-2(1H)-yl)-4-azaspiro[2.5]octane-4-carboxylate (80 mg, 0.173 mmol, 1 equiv) and bis(pinacolato)diboron (54 mg, 0.212 mmol, 1.2 equiv) in dioxane (5 mL) was added KOAc (35 mg, 0.354 mmol, 2 equiv) and Pd(dppf)Cl₂ (13 mg, 0.018 mmol, 0.1 equiv) in portions at room temperature under nitrogen atmosphere. The resulting mixture was stirred for 4 h at 100° C. under nitrogen atmosphere, then cooled to room temperature. To the resulting mixture was added 5-bromo-2,7-dimethylpyrazolo[4,3-b]pyridine (59 mg, 0.259 mmol, 1.5 equiv), K₃PO₄ (110 mg, 0.519 mmol, 3 equiv), Pd(dppf)Cl₂ (12 mg, 0.017 mmol, 0.1 equiv), and H₂O (1 mL) at room temperature under nitrogen atmosphere. The resulting mixture was stirred for additional 4 h at 100° C., then cooled to room temperature. The reaction mixture was quenched with water (10 mL) and extracted with ethyl acetate (2×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with PE/EA (2:1) to afford tert-butyl 7-(6-{2,7-dimethylpyrazolo[4,3-b]pyridin-5-yl}-8-fluoro-1-oxoisoquinolin-2-yl)-4-azaspiro[2.5]octane-4-carboxylate (70 mg, 76%) as a solid. LCMS (ES, m/z): 518 [M+H]⁺.

### Synthesis of Compound 431

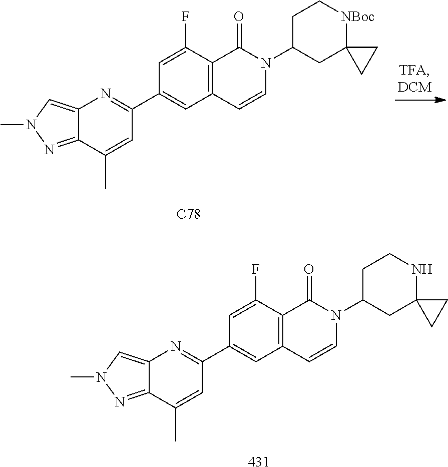 [SMILES: Cc1cc(-c2cc(F)c3c(=O)n(C4CCN(C(=O)OC(C)(C)C)C5(CC5)C4)ccc3c2)nc2cn(C)nc12 ; "C78" ; Cc1cc(-c2cc(F)c3c(=O)n(C4CCNC5(CC5)C4)ccc3c2)nc2cn(C)nc12 ; "431"]

A mixture of tert-butyl 7-(6-{2,7-dimethylpyrazolo[4,3-b]pyridin-5-yl}-8-fluoro-1-oxoisoquinolin-2-yl)-4-azaspiro[2.5]octane-4-carboxylate (38 mg, 0.073 mmol, 1 equiv) and trifluoroacetic acid (3 mL) in DCM (3 mL) was stirred for 2 h at room temperature. The resulting mixture was concentrated under reduced pressure to give a residue. The residue was neutralized to pH 8 with ammonia and purified by reverse flash chromatography (Condition 2, Gradient 11) to afford 2-{4-azaspiro[2.5]octan-7-yl}-8-fluoro-6-{2-methylimidazo[1,2-a]pyrazin-6-yl}isoquinolin-1-one (8 mg, 37%) as a solid. LCMS (ES, m/z): 417 [M+H]¹H NMR (300 MHz, DMSO-d₆) δ 8.71 (s, 1H), 8.24 (s, 1H), 8.00-7.87 (m, 2H), 7.57 (d, J=7.5 Hz, 1H), 6.80 (d, J=7.4 Hz, 1H), 5.05 (s, 1H), 4.26 (s, 3H), 3.02 (d, J=12.8 Hz, 1H), 2.65 (s, 2H), 2.58 (s, 2H), 2.20 (t, J=11.9 Hz, 1H), 1.74 (s, 2H), 1.13 (d, J=13.2 Hz, 1H), 0.59 (t, J=5.9 Hz, 1H), 0.45 (d, J=6.7 Hz, 2H), 0.41-0.31 (m, 1H). ¹⁹F NMR (282 MHz, DMSO-d6) δ−110.47.

### Example 128: Synthesis of Compound 361

### Synthesis of Intermediate C79

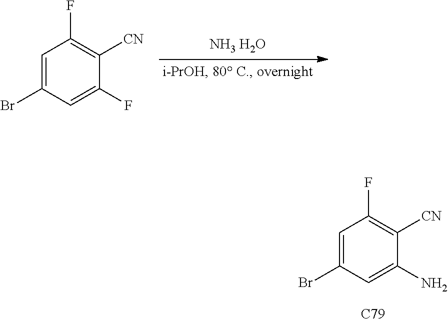 [SMILES: N#Cc1c(F)cc(Br)cc1F ; "NH3 H2O" ; N#Cc1c(N)cc(Br)cc1F ; "C79"]

A mixture of 4-bromo-2,6-difluorobenzonitrile (2.0 g, 9.174 mmol, 1.0 equiv), i-PrOH (5 mL), and NH₃·H₂O (8 mL) was stirred overnight at 80° C. The reaction mixture was concentrated under reduced pressure, then washed with water (10 mL) and filtered under vacuum to afford 2-amino-4-bromo-6-fluorobenzonitrile (1.8 g, 91%) as a solid. LCMS (ES, m/z): 215 [M+H]⁺.

### Synthesis of Intermediate C80

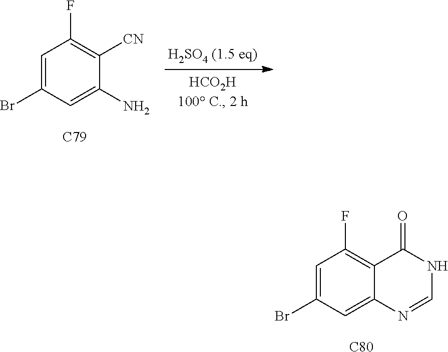 [SMILES: N#Cc1c(N)cc(Br)cc1F ; "H2SO4 (1.5 eq)" ; "C79" ; O=c1[nH]cnc2cc(Br)cc(F)c12 ; "C80"]

A mixture of 2-amino-4-bromo-6-fluorobenzonitrile (1.8 g, 8.371 mmol, 1 equiv), HCOOH (20 mL), and H₂SO₄ (1.23 g, 12.556 mmol, 1.5 equiv) was stirred for 2 h at 100° C. The reaction mixture was poured into ice water, stirred for 15 minutes, and a precipitate formed. The precipitate was filtered, washed with petroleum ether, and drained under vacuum to afford 7-bromo-5-fluoro-3H-quinazolin-4-one (1.4 g, 69%) as a solid. LCMS (ES, m/z): 243 [M+H]⁺.

### Synthesis of Intermediate C81

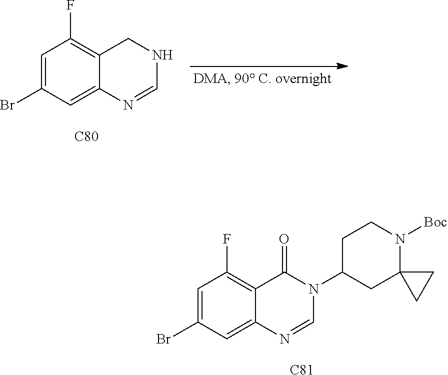 [SMILES: Fc1cc(Br)cc2c1CNC=N2 ; "C80" ; CC(C)(C)OC(=O)N1CCC(n2cnc3cc(Br)cc(F)c3c2=O)CC12CC2 ; "C81"]

A mixture of 7-bromo-5-fluoro-3H-quinazolin-4-one (500 mg, 2.057 mmol, 1.0 equiv), tert-butyl 7-(methanesulfonyloxy)-4-azaspiro[2.5]octane-4-carboxylate (1256.57 mg, 4.114 mmol, 2.0 equiv), DMA (5 mL), and potassium carbonate (1145.62 mg, 8.228 mmol, 4.0 equiv) was stirred overnight at 90° C. The resulting mixture was diluted with water (20 mL) and extracted with ethyl acetate (3×10 mL). The organic layers were combined, washed with brine (1×10 mL), dried over anhydrous Na₂SO₄, and concentrated under reduced pressure to give a residue. The residue was purified by reverse phase flash chromatography (Condition, Gradient 1) to afford tert-butyl 7-(7-bromo-5-fluoro-4-oxoquinazolin-3-yl)-4-azaspiro[2.5]octane-4-carboxylate (110 mg, 12%) as a solid. LCMS (ES, m/z): 452 [M+H]⁺.

### Synthesis of Intermediate C82

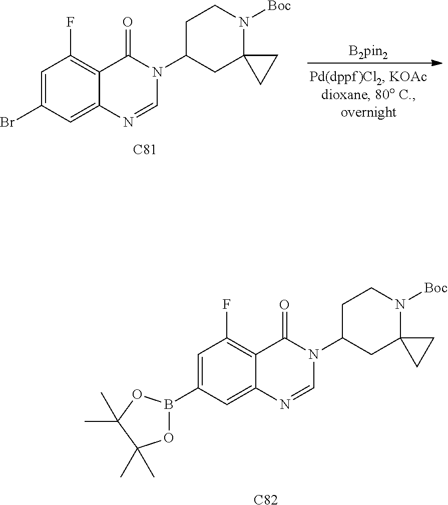 [SMILES: CC(C)(C)OC(=O)N1CCC(n2cnc3cc(Br)cc(F)c3c2=O)CC12CC2 ; "C81" ; CC(C)(C)OC(=O)N1CCC(n2cnc3cc(B4OC(C)(C)C(C)(C)O4)cc(F)c3c2=O)CC12CC2 ; "C82"]

Tert-butyl 7-(7-bromo-5-fluoro-4-oxoquinazolin-3-yl)-4-azaspiro[2.5]octane-4-carboxylate (60 mg, 0.133 mmol, 1.0 equiv), 4,4,5,5-tetramethyl-2-(tetramethyl-1,3,2-dioxaborolan-2-yl)-1,3,2-dioxaborolane (40.42 mg, 0.160 mmol, 1.2 equiv), AcOK (39.06 mg, 0.399 mmol, 3.0 equiv), dioxane (1 mL), and Pd(dppf)Cl₂CH₂Cl₂ (10.81 mg, 0.013 mmol, 0.1 equiv) were combined at room temperature under nitrogen atmosphere. The resulting mixture was stirred overnight at 80° C. LCMS (ES, m/z): 418 [M+H]⁺.

### Synthesis of Intermediate C83

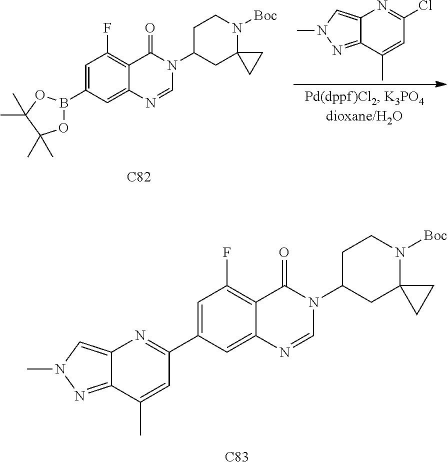 [SMILES: CC(C)(C)OC(=O)N1CCC(n2cnc3cc(B4OC(C)(C)C(C)(C)O4)cc(F)c3c2=O)CC12CC2 ; Cc1cc(Cl)nc2cn(C)nc12 ; "C82" ; Cc1cc(-c2cc(F)c3c(=O)n(C4CCN(C(=O)OC(C)(C)C)C5(CC5)C4)cnc3c2)nc2cn(C)nc12 ; "C83"]

To the stirred mixture of C82 was added water (0.2 ml), 5-chloro-2,7-dimethylpyrazolo[4,3-b]pyridine (21.82 mg, 0.120 mmol, 1.0 equiv), K₃PO₄ (25.50 mg, 0.120 mmol, 1.0 equiv), and Pd(dppf)Cl₂CH₂Cl₂ (9.79 mg, 0.012 mmol, 0.1 equiv) at room temperature under nitrogen atmosphere. The resulting mixture was stirred overnight at 100° C. under nitrogen atmosphere, then diluted with water (2 mL) and extracted with ethyl acetate (3×5 mL). The organic layers were combined, washed with brine (1×10 mL), dried over anhydrous Na₂SO₄, filtered. The filtrate was concentrated under reduced pressure to give a residue. The residue was purified by silica gel column chromatography, eluted with CH₂Cl₂/MeOH (10:1) to afford tert-butyl 7-(7-{2,7-dimethylpyrazolo[4,3-b]pyridin-5-yl}-5-fluoro-4-oxoquinazolin-3-yl)-4-azaspiro[2.5]octane-4-carboxylate (40 mg, 64%) as a solid. LCMS (ES, m/z): 519 [M+H]⁺.

### Synthesis of Compound 361

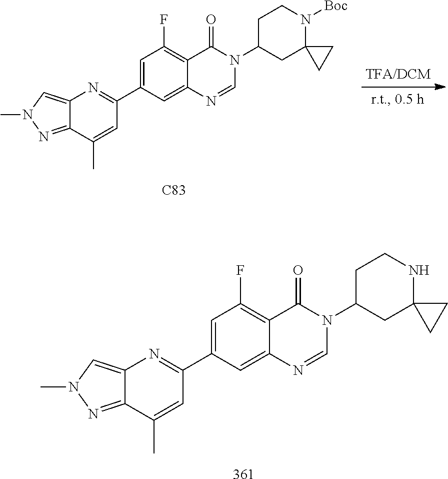 [SMILES: Cc1cc(-c2cc(F)c3c(=O)n(C4CCN(C(=O)OC(C)(C)C)C5(CC5)C4)cnc3c2)nc2cn(C)nc12 ; "C83" ; Cc1cc(-c2cc(F)c3c(=O)n(C4CCNC5(CC5)C4)cnc3c2)nc2cn(C)nc12 ; "361"]

Tert-butyl 7-(7-{2,7-dimethylpyrazolo[4,3-b]pyridin-5-yl}-5-fluoro-4-oxoquinazolin-3-yl)-4-azaspiro[2.5]octane-4-carboxylate (40 mg, 0.077 mmol, 1 equiv), DCM (1 mL) and TFA (1 mL, 13.463 mmol, 174.55 equiv) were combined at room temperature. The resulting mixture was stirred for 30 min at room temperature, then concentrated under reduced pressure to give a residue. The residue was purified by reverse phase flash chromatography (Condition 4, Gradient 1) to afford 3-{4-azaspiro[2.5]octan-7-yl}-7-{2,7-dimethylpyrazolo[4,3-b]pyridin-5-yl}-5-fluoroquinazolin-4-one (12 mg, 37%) as a solid. LCMS (ES, m/z): 419 [M+H]⁺. ¹H NMR (400 MHz, DMSO-d₆) δ 8.73 (s, 1H), 8.48 (s, 1H), 8.23 (d, J=1.6 Hz, 1H), 8.04 (dd, J=12.7, 1.7 Hz, 1H), 7.96 (d, J=1.2 Hz, 1H), 4.88 (ddt, J=12.1, 8.0, 4.2 Hz, 1H), 4.26 (s, 3H), 3.07-2.99 (m, 1H), 2.72 (d, J=11.1 Hz, 1H), 2.65 (d, J=1.0 Hz, 3H), 2.35 (t, J=11.8 Hz, 1H), 2.09 (s, 1H), 1.98-1.80 (m, 2H), 1.27-1.19 (m, 1H), 0.59 (td, J=8.7, 4.6 Hz, 1H), 0.46 (q, J=5.2 Hz, 2H), 0.42-0.33 (m, 1H).

### Example 129: Synthesis of Compound 358

### Synthesis of Intermediate C84

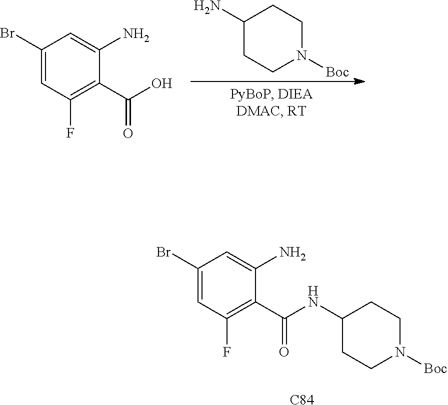 [SMILES: Nc1cc(Br)cc(F)c1C(=O)O ; CC(C)(C)OC(=O)N1CCC(N)CC1 ; CC(C)(C)OC(=O)N1CCC(NC(=O)c2c(N)cc(Br)cc2F)CC1 ; "C84"]

A mixture of 2-amino-4-bromo-6-fluorobenzoic acid (2 g, 8.546 mmol, 1 equiv), PyBOP (6.67 g, 12.819 mmol, 1.5 equiv), DMAC (40 mL), and DIEA (3.31 g, 25.610 mmol, 3.00 equiv) was stirred for 0.5 h at room temperature. To the reaction mixture was added tert-butyl 4-aminopiperidine-1-carboxylate (2.05 g, 10.255 mmol, 1.2 equiv). The resulting mixture was stirred for an additional 2 h at 25° C. The reaction mixture was quenched with water (10 mL) and extracted with ethyl acetate (2×10 mL). The resulting liquid was dried over Na₂SO₄ and filtered. The filtrate was concentrated under reduced pressure to afford tert-butyl 4-(2-amino-4-bromo-6-fluorobenzamido)piperidine-1-carboxylate (2.7 g, 76%) as a solid. LCMS (ES, m/z): 416 [M+H]⁺.

### Synthesis of Intermediate C85

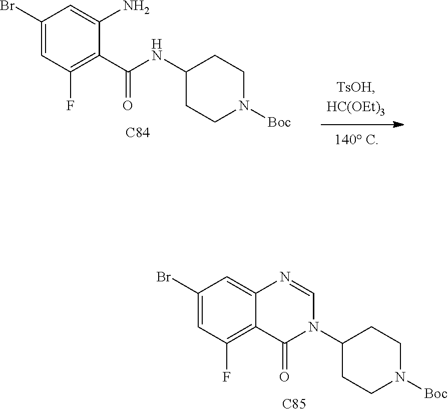 [SMILES: CC(C)(C)OC(=O)N1CCC(NC(=O)c2c(N)cc(Br)cc2F)CC1 ; "C84" ; CC(C)(C)OC(=O)N1CCC(n2cnc3cc(Br)cc(F)c3c2=O)CC1 ; "C85"]

Tert-butyl 4-(2-amino-4-bromo-6-fluorobenzamido) piperidine-1-carboxylate (2 g, 4.804 mmol, 1 equiv), triethyl orthoformate (7.12 g, 48.040 mmol, 10 equiv), and para-toluene sulfonate (0.08 g, 0.480 mmol, 0.1 equiv) were combined at 25° C. The resulting mixture was stirred for 5 h at 140° C., then cooled to 25° C. The reaction mixture was concentrated under reduced pressure to give a residue. The residue was purified by silica gel column chromatography, eluted with PE/EA (1:1) to afford tert-butyl 4-(7-bromo-5-fluoro-4-oxoquinazolin-3-yl)piperidine-1-carboxylate (2 g, 98%) as a solid. LCMS (ES, m/z): 426 [M+H]⁺.

### Synthesis of Intermediate C86

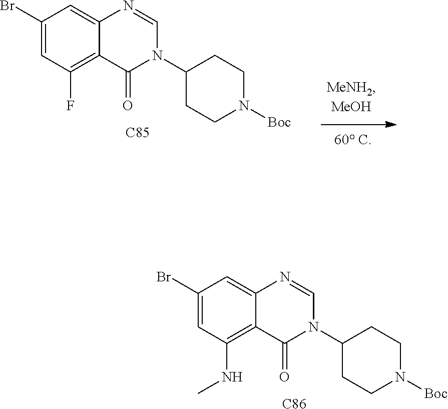 [SMILES: CC(C)(C)OC(=O)N1CCC(n2cnc3cc(Br)cc(F)c3c2=O)CC1 ; "C85" ; CNc1cc(Br)cc2ncn(C3CCN(C(=O)OC(C)(C)C)CC3)c(=O)c12 ; "C86"]

Tert-butyl 4-(7-bromo-5-fluoro-4-oxoquinazolin-3-yl) piperidine-1-carboxylate (1 g, 2.346 mmol, 1 equiv), methylamine (0.36 g, 11.730 mmol, 5 equiv), and methanol (10 mL) were combined at 25° C. The resulting mixture was stirred for 5 h at 60° C., then cooled to 25° C. The resulting mixture was concentrated under reduced pressure to give a residue. The residue was purified by silica gel column chromatography, eluted with PE/EA (1:1) to afford tert-butyl 4-[7-bromo-5-(methylamino)-4-oxoquinazolin-3-yl]piperidine-1-carboxylate (1 g, 97%) as a solid. LCMS (ES, m/z): 437 [M+H]⁺.

### Synthesis of Intermediate C87

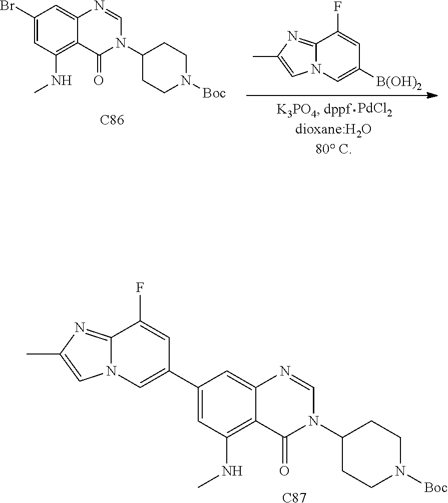

To a solution of tert-butyl 4-[7-bromo-5-(methylamino)-4-oxoquinazolin-3-yl]piperidine-1-carboxylate (100 mg, 0.229 mmol, 1 equiv) and 8-fluoro-2-methylimidazo[1,2-a]pyridin-6-ylboronic acid (53.22 mg, 0.275 mmol, 1.2 equiv) in dioxane (2 mL) and H₂O (0.2 mL) was added K₃PO₄ (145.61 mg, 0.687 mmol, 3 equiv) and Pd(dppf)Cl₂ (16.73 mg, 0.023 mmol, 0.1 equiv). After stirring for 6 h at 80° C. under a nitrogen atmosphere, the resulting mixture was concentrated under reduced pressure to give a residue. The residue was purified by silica gel column chromatography, eluted with THF/EA (1:1) to afford tert-butyl 4-(7-{8-fluoro-2-methylimidazo[1,2-a]pyridin-6-yl}-5-(methylamino)-4-oxoquinazolin-3-yl) piperidine-1-carboxylate (65 mg, 56%) as a solid. LCMS (ES, m/z): 507 [M+H]⁺.

### Synthesis of Compound 358

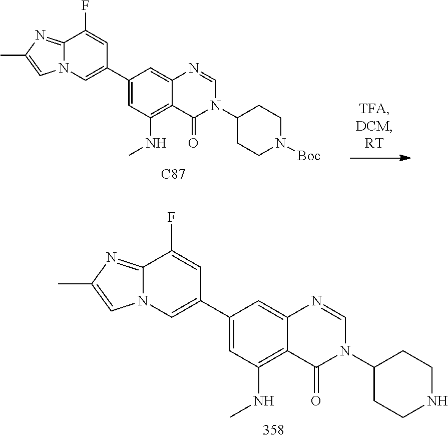

Tert-butyl 4-(7-{8-fluoro-2-methylimidazo[1,2-a]pyridin-6-yl}-5-(methyl amino)-4-oxoquinazolin-3-yl)piperidine-1-carboxylate (65 mg, 0.128 mmol, 1 equiv), DCM (1 mL) and trifluoroacetaldehyde (1 mL) were combined at room temperature. The resulting mixture was stirred for 1 h at room temperature, then concentrated under reduced pressure to give a residue. The residue was purified by Prep-HPLC (Condition 6, Gradient 1, Gradient 2) to afford 7-{8-fluoro-2-methylimidazo[1,2-a]pyridine-6-yl}-5-(methylamino)-3-(piperidin-4-yl)quinazolin-4-one (8 mg, 15%) as a solid. LCMS (ES, m/z): 407 [M+H]⁺. ¹H NMR (300 MHz, Methanol-d4) δ 9.05 (d, J=1.4 Hz, 1H), 8.39 (s, 1H), 8.29-8.06 (m, 2H), 7.07 (d, J=1.7 Hz, 1H), 6.89 (d, J=1.7 Hz, 1H), 3.73-3.53 (m, 2H), 3.24 (dd, J=13.3, 3.0 Hz, 2H), 3.07 (s, 3H), 2.71-2.57 (m, 3H), 2.48 (s, 2H), 2.24 (d, J=12.9 Hz, 2H).

### Example 130: Synthesis of Compound 359

### Synthesis of Intermediate C88

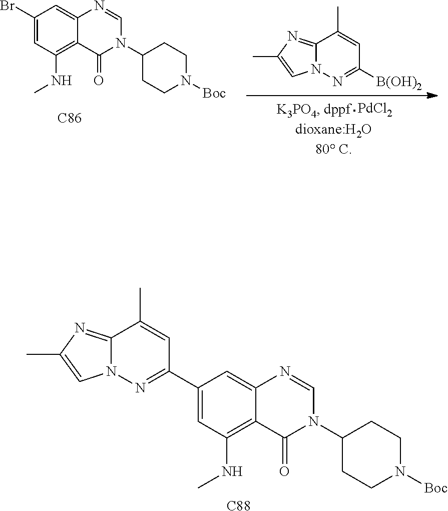

To a solution of tert-butyl 4-[7-bromo-5-(methylamino)-4-oxoquinazolin-3-yl]piperidine-1-carboxylate (100 mg, 0.229 mmol, 1 equiv) and 2,8-dimethylimidazo[1,2-b]pyridazin-6-ylboronic acid (52.41 mg, 0.275 mmol, 1.2 equiv) in dioxane (2 mL) and H₂O (0.2 mL) was added K₃PO₄ (145.61 mg, 0.687 mmol, 3 equiv) and Pd(dppf)Cl₂ (16.73 mg, 0.023 mmol, 0.1 equiv). The reaction mixture was stirred for 6 h at 80° C. under a nitrogen atmosphere, then concentrated under reduced pressure to give a residue. The residue was purified by silica gel column chromatography, eluted with THF/EA (1:1) to afford tert-butyl 4-(7-{2,8-dimethylimidazo[1,2-b]pyridazin-6-yl}-5-(methylamino)-4-oxoquinazolin-3-yl)piperidine-1-carboxylate (57 mg, 50%) as a solid. LCMS (ES, m/z): 504 [M+H]⁺.

### Synthesis of Compound 359

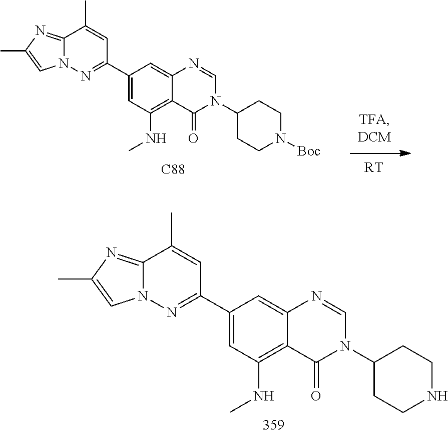

Tert-butyl 4-(7-{2,8-dimethylimidazo[1,2-b]pyridazin-6-yl}-5-(methylamino)-4-oxoquinazolin-3-yl)piperidine-1-carboxylate (57 mg, 0.113 mmol, 1 equiv), DCM (1 mL) and trifluoroacetaldehyde (1 mL) were combined at room temperature. The resulting mixture was stirred for 1 h at room temperature, then concentrated under reduced pressure to give a residue. The residue was purified by Prep-HPLC with the following conditions (Condition 6, Gradient 1, Gradient 2) to afford 7-{2,8-dimethylimidazo[1,2-b]pyridazin-6-yl}-5-(methyl amino)-3-(piperid in-4-yl)quinazolin-4-one (25 mg, 55%) as a solid. LCMS (ES, m/z): 404 [M+H]⁺. ¹H NMR (300 MHz, Methanol-d4) δ 8.41-8.17 (m, 3H), 7.39 (d, J=1.7 Hz, 1H), 7.22 (d, J=1.6 Hz, 1H), 3.75-3.52 (m, 2H), 3.25 (dd, J=13.2, 3.0 Hz, 2H), 3.06 (s, 3H), 2.80 (d, J=1.1 Hz, 3H), 2.67 (d, J=1.0 Hz, 3H), 2.51 (dd, J=12.8, 4.0 Hz, 2H), 2.24 (d, J=13.5 Hz, 2H).

### Example 131: Synthesis of Compound 360 Synthesis of Intermediate C89

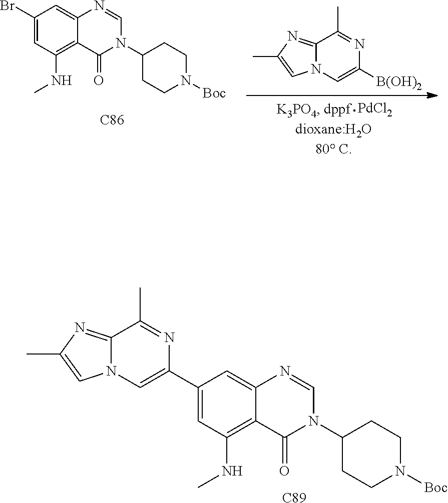

To a solution of tert-butyl 4-[7-bromo-5-(methylamino)-4-oxoquinazolin-3-yl]piperidine-1-carboxylate (100 mg, 0.229 mmol, 1 equiv) and 2,8-dimethylimidazo[1,2-a]pyrazin-6-ylboronic acid (52.41 mg, 0.275 mmol, 1.2 equiv) in dioxane (2 mL) and H₂O (0.2 mL) was added K₃PO₄ (145.61 mg, 0.687 mmol, 3 equiv) and Pd(dppf)Cl₂ (16.73 mg, 0.023 mmol, 0.1 equiv). After stirring for 6 h at 80° C. under a nitrogen atmosphere, the resulting mixture was concentrated under reduced pressure to give a residue. The residue was purified by silica gel column chromatography, eluted with THF/EA (1:1) to afford tert-butyl 4-(7-{2,8-dimethylimidazo[1,2-a]pyrazin-6-yl}-5-(methylamino)-4-oxoquinazolin-3-yl) piperidine-1-carboxylate (62 mg, 54%) as a solid. LCMS (ES, m/z): 504 [M+H]⁺.

### Synthesis of Compound 360

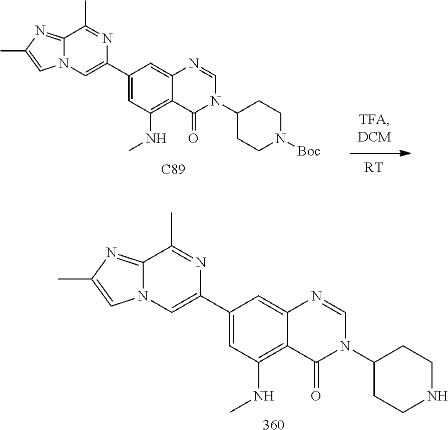

Tert-butyl 4-(7-{2,8-dimethylimidazo[1,2-a]pyrazine-6-yl}-5-(methyl amino)-4-oxoquinazolin-3-yl)piperidine-1-carboxylate (62 mg, 0.123 mmol, 1 equiv), DCM (1 mL) and trifluoroacetaldehyde (1 mL) were combined at room temperature. The resulting mixture was stirred for 1 h at room temperature, then concentrated under reduced pressure to give a residue. The residue was purified by Prep-HPLC (Condition 7, Gradient 1) to afford 7-{2,8-dimethylimidazo[1,2-a]pyrazine-6-yl}-5-(methylamino)-3-(piperidin-4-yl)quinazolin-4-one (20 mg, 40%) as a solid. LCMS (ES, m/z): 404 [M+H]⁺. ¹H NMR (300 MHz, Methanol-d4) δ 9.15 (s, 1H), 8.52 (s, 1H), 8.08 (s, 1H), 7.31 (s, 1H), 7.16 (d, J=3.4 Hz, 1H), 4.90 (s, 1H), 3.66 (d, J=12.6 Hz, 2H), 3.31-3.15 (m, 2H), 3.03 (s, 3H), 2.92 (s, 3H), 2.62 (s, 3H), 2.50 (qd, J=13.1, 3.9 Hz, 2H), 2.30 (s, 2H).

### Example 132: Synthesis of Compound 461

### Synthesis of Intermediate C90

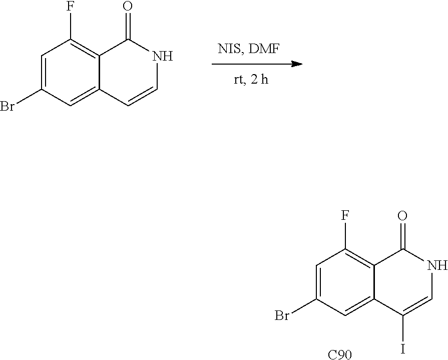 [SMILES: O=c1[nH]ccc2cc(Br)cc(F)c12 ; O=c1[nH]cc(I)c2cc(Br)cc(F)c12 ; "C90"]

A mixture of 6-bromo-8-fluoro-2H-isoquinolin-1-one (6 g, 24.789 mmol, 1 equiv) and NIS (8.37 g, 37.184 mmol, 1.5 equiv) in DMF (50 mL) was stirred for 2 h at 80° C. The reaction mixture was cooled to room temperature, then quenched with water (100 mL) at 0° C. A solid precipitated that was collected by filtration and washed with water (3×10 mL). The resulting solid was dried in an oven under reduced pressure to afford 6-bromo-8-fluoro-4-iodo-2H-isoquinolin-1-one (5.5 g, 60%). LCMS (ES, m/z): 368[M+H]⁺.

### Synthesis of Intermediate C91

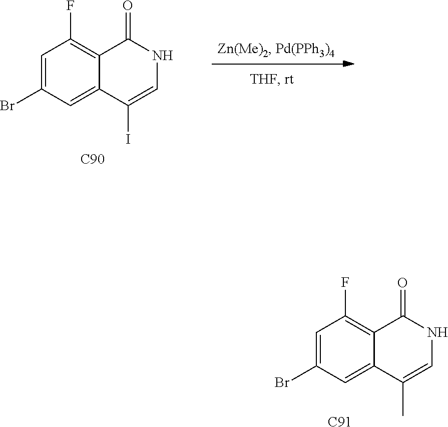

To a stirred mixture of 6-bromo-8-fluoro-4-iodo-2H-isoquinolin-1-one (6.6 g, 17.93 mmol, 1 equiv) and Pd(PPh₃)₄(414.5 mg, 0.359 mmol, 0.02 equiv) in THF (70 mL, 814.565 mmol, 45.41 equiv) was added dimethylzinc (35.9 mL, 35.876 mmol, 2 equiv, 1 M in THF) dropwise at room temperature under nitrogen atmosphere. The resulting mixture was stirred for an additional 5 h at room temperature, then quenched with a solution of HOAc in water (50 mL, 1 mol/L). A precipitate formed that was collected by filtration and washed with THF (3×20 mL) to afford 6-bromo-8-fluoro-4-methyl-2H-isoquinolin-1-one (1.5 g, 33%) as a solid. LCMS (ES, m/z): 256 [M+H]⁺.

### Synthesis of Intermediate C92

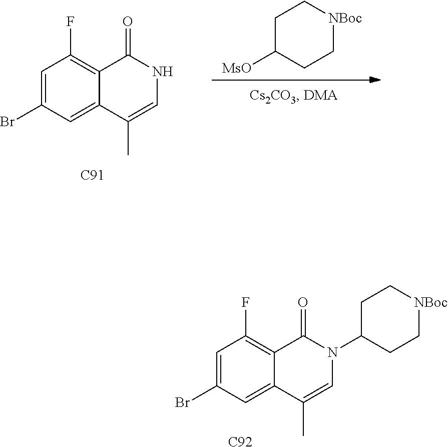 [SMILES: Cc1c[nH]c(=O)c2c(F)cc(Br)cc12 ; CC(C)(C)OC(=O)N1CCC(OS(C)(=O)=O)CC1 ; "C91" ; Cc1cn(C2CCN(C(=O)OC(C)(C)C)CC2)c(=O)c2c(F)cc(Br)cc12 ; "C92"]

To a stirred mixture of 6-bromo-8-fluoro-4-methyl-2H-isoquinolin-1-one (200 mg, 0.781 mmol, 1 equiv) and tert-butyl 4-(methanesulfonyloxy)piperidine-1-carboxylate (436 mg, 1.562 mmol, 2 equiv) in DMA (5 mL) was added Cs₂CO₃ (763 mg, 2.343 mmol, 3 equiv) at room temperature. The resulting mixture was stirred for 2 h at 110° C. The reaction mixture was quenched with water (20 mL) at room temperature, then extracted with ethyl acetate (2×30 mL). The organic layers were combined, washed with brine (3×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(6-bromo-8-fluoro-4-methyl-1-oxoisoquinolin-2-yl)piperidine-1-carboxylate (130 mg, 38%) as a solid. LCMS (ES, m/z): 439 [M+H]⁺.

### Synthesis of Intermediate C93

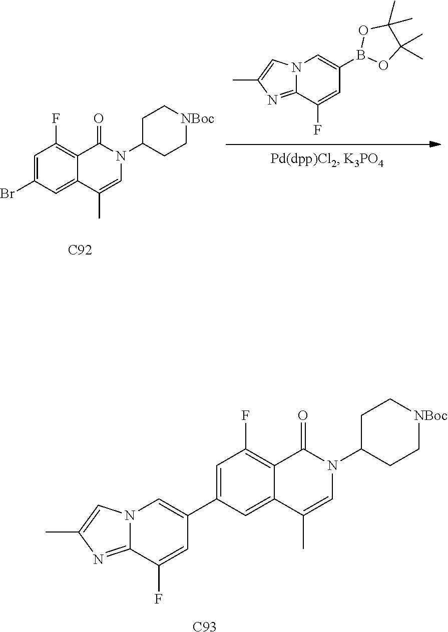

To a stirred mixture of tert-butyl 4-(6-bromo-8-fluoro-4-methyl-1-oxoisoquinolin-2-yl)piperidine-1-carboxylate (200 mg, 0.455 mmol, 1 equiv) and 8-fluoro-2-methyl-6-(4,4,5,5-tetramethyl-1,3,2-dioxaborolan-2-yl)imidazo[1,2-a]pyridine (150 mg, 0.546 mmol, 1.2 equiv) in H₂O (2 mL) and dioxane (10 mL) was added K₃PO₄ (193 mg, 0.910 mmol, 2 equiv) and Pd(dppf)Cl₂ (33 mg, 0.046 mmol, 0.1 equiv) in portions at room temperature under nitrogen atmosphere. The resulting mixture was stirred for 3 h at 80° C. The reaction mixture was quenched with water (20 mL) at room temperature, then extracted with ethyl acetate (2×20 mL). The organic layers were combined, washed with brine (2×20 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(8-fluoro-6-{8-fluoro-2-methylimidazo[1,2-a]pyridin-6-yl}-4-methyl-1-oxoisoquinolin-2-yl)piperidine-1-carboxylate (120 mg, 52%) as a solid. LCMS (ES, m/z): 509 [M+H]⁺.

### Synthesis of Compound 461

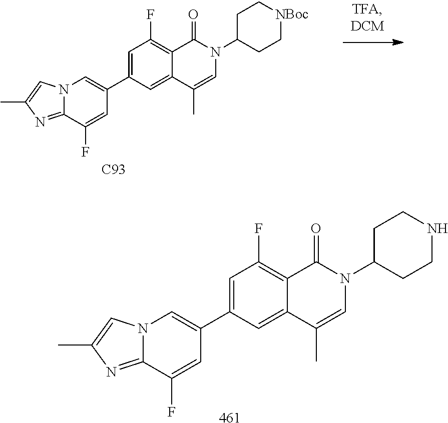 [SMILES: Cc1cn2cc(-c3cc(F)c4c(=O)n(C5CCNCC5)cc(C)c4c3)cc(F)c2n1 ; "C93" ; Cc1cn2cc(-c3cc(F)c4c(=O)n(C5CCNCC5)cc(C)c4c3)cc(F)c2n1 ; "461" ; "Boc"]

A mixture of tert-butyl 4-(8-fluoro-6-{8-fluoro-2-methylimidazo[1,2-a]pyridin-6-yl}-4-methyl-1-oxoisoquinolin-2-yl)piperidine-1-carboxylate (120 mg, 0.236 mmol, 1 equiv) and trifluoroacetic acid (5 mL) in DCM (20 mL) was stirred for 2 h at room temperature. The resulting mixture was concentrated under reduced pressure to give a residue. The residue was purified by Prep-HPLC (Condition 8, Gradient 1) to afford 8-fluoro-6-{8-fluoro-2-methylimidazo[1,2-a]pyridin-6-yl}-4-methyl-2-(piperidin-4-yl)isoquinolin-1-one (30 mg, 31%) as a solid. LCMS (ES, m/z): 408 [M+H]⁺. ¹H NMR (300 MHz, DMSO-d₆) δ 9.03 (d, J=1.5 Hz, 1H), 7.85 (dd, J=3.1, 1.0 Hz, 1H), 7.81-7.62 (m, 3H), 7.44 (s, 1H), 4.91-4.77 (m, 1H), 3.10 (d, J=12.0 Hz, 2H), 2.63 (t, J=11.6 Hz, 2H), 2.43-2.32 (m, 7H), 1.88-1.61 (m, 4H). ¹⁹F NMR (282 MHz, DMSO-d6) δ−109.70, −132.07.

### Example 133: Synthesis of Compound 440

### Synthesis of Intermediate C94

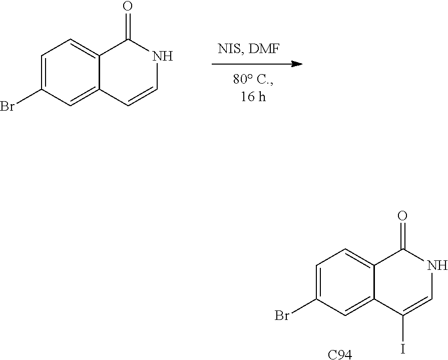 [SMILES: O=c1[nH]ccc2cc(Br)ccc12 ; O=c1[nH]cc(I)c2cc(Br)ccc12 ; "C94"]

A mixture of 6-bromo-2H-isoquinolin-1-one (1 g, 4.463 mmol, 1 equiv) and NIS (1.51 g, 6.694 mmol, 1.5 equiv) in DMF (20 mL) was stirred for 12 h at 80° C. under nitrogen atmosphere, then cooled to room temperature. The resulting mixture was poured into the water (100 mL) and a precipitate formed. The precipitate was collected by filtration, washed with H₂O (3×10 mL), and dried to afford 6-bromo-4-iodo-2H-isoquinolin-1-one (0.9 g, 58%) as a solid. LCMS (ES, m/z): 350 [M+H]⁺.

### Synthesis of Intermediate C95

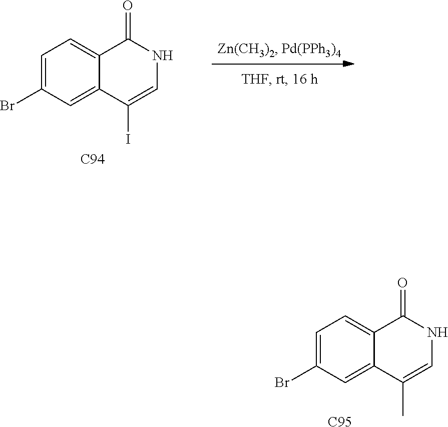

To a stirred mixture of 6-bromo-4-iodo-2H-isoquinolin-1-one (10 g, 28.575 mmol, 1 equiv) and Pd(PPh₃)₄(3.30 g, 2.857 mmol, 0.1 equiv) in THF (60 mL) was added Zn(CH₃)₂(115 mL, 57.15 mmol, 2.0 equiv, 0.5 mol/L in hexane) at room temperature. The reaction mixture was stirred for 16 h, then quenched with HOAc (100 mL, 1 mol/L in water) at room temperature. A precipitate formed that was collected by filtration and washed with H₂O (3×10 mL) to afford 6-bromo-4-methyl-2H-isoquinolin-1-one (3.5 g, 51%) as a solid. LCMS (ES, m/z): 238 [M+H]⁺.

### Synthesis of Intermediate C96

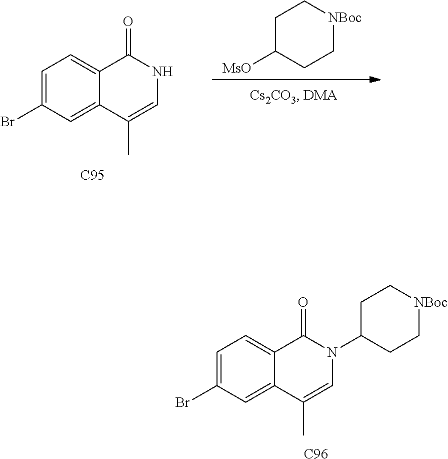 [SMILES: Cc1c[nH]c(=O)c2ccc(Br)cc12 ; CC(C)(C)OC(=O)N1CCC(OS(C)(=O)=O)CC1 ; "C95" ; Cc1cn(C2CCN(C(=O)OC(C)(C)C)CC2)c(=O)c2ccc(Br)cc12 ; "C96"]

To a stirred mixture of 6-bromo-4-methyl-2H-isoquinolin-1-one (500 mg, 2.1 mmol, 1 equiv) and tert-butyl 4-(methanesulfonyloxy)piperidine-1-carboxylate (645 mg, 2.31 mmol, 1.1 equiv) in DMA (5 mL) was added Cs₂CO₃ (2052 mg, 6.3 mmol, 3 equiv) in portions at room temperature. The resulting mixture was stirred for 2 h at 110° C., then cooled to room temperature. The reaction mixture was quenched with water (30 mL) and extracted with ethyl acetate (2×50 mL). The organic layers were combined, washed with brine (2×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(6-bromo-4-methyl-1-oxoisoquinolin-2-yl)piperidine-1-carboxylate (380 mg, 43%) as a solid. LCMS (ES, m/z):421 [M+H]. ⁺.

### Synthesis of Intermediate C97

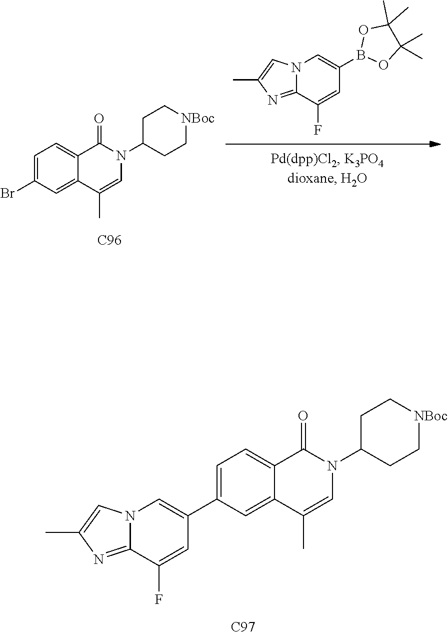

To a stirred mixture of tert-butyl 4-(6-bromo-4-methyl-1-oxoisoquinolin-2-yl)piperidine-1-carboxylate (200 mg, 0.475 mmol, 1.0 equiv), K₃PO₄ (201 mg, 0.95 mmol, 2 equiv), and (2-{8-fluoro-2-methylimidazo[1,2-a]pyridin-6-yl}-4,5,5-trimethyl-1,3,2-dioxaborolan-4-yl)methylium (156 mg, 0.570 mmol, 1.2 equiv) in dioxane (3 mL) was added H₂O (0.5 mL) and Pd(dppf)Cl₂ (34.7 mg, 0.048 mmol, 0.1 equiv) in portions at room temperature under nitrogen atmosphere. The resulting mixture was stirred for 3 h at 80° C., then quenched with water (10 mL) at room temperature and extracted with ethyl acetate (2×10 mL). The organic layers were combined, washed with brine (2×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(6-{8-fluoro-2-methylimidazo[1,2-a]pyridin-6-yl}-4-methyl-1-oxoisoquinolin-2-yl)piperidine-1-carboxylate (150 mg, 64%) as a solid. LCMS (ES, m/z): 491[M+H]⁺.

### Synthesis of Compound 440

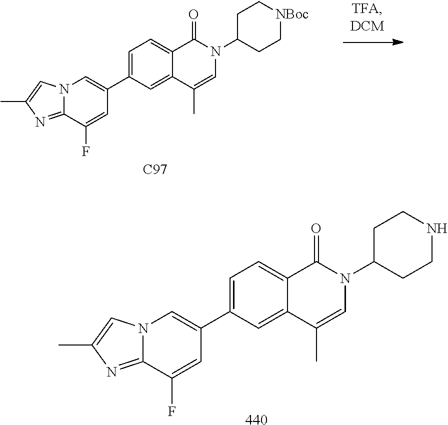 [SMILES: Cc1cn2cc(-c3ccc4c(=O)n(C5CCNCC5)cc(C)c4c3)cc(F)c2n1 ; "C97" ; Cc1cn2cc(-c3ccc4c(=O)n(C5CCNCC5)cc(C)c4c3)cc(F)c2n1 ; "440" ; "Boc"]

A mixture of tert-butyl 4-(6-{8-fluoro-2-methylimidazo[1,2-a]pyridin-6-yl}-4-methyl-1-oxoisoquinolin-2-yl)piperidine-1-carboxylate (100 mg, 0.204 mmol, 1 equiv) and trifluoroacetic acid (116.2 mg, 1.020 mmol, 5 equiv) in DCM (10 mL) was stirred for 2 h at 0° C. The resulting mixture was concentrated under vacuum to give a residue. The residue was purified by reverse flash chromatography (Condition 2, Gradient 12) to afford 6-{8-fluoro-2-methylimidazo[1,2-a]pyridin-6-yl}-4-methyl-2-(piperidin-4-yl)isoquinolin-1-one (30 mg, 38%) as a solid. LCMS (ES, m/z): 390 [M+H]⁺. ¹H NMR (300 MHz, Methanol-d₄) δ 8.75 (d, J=1.5 Hz, 1H), 8.50 (d, J=8.5 Hz, 1H), 7.99 (d, J=1.8 Hz, 1H), 7.88 (dd, J=8.5, 1.8 Hz, 1H), 7.82 (d, J=3.1 Hz, 1H), 7.59 (dd, J=12.1, 1.4 Hz, 1H), 7.36 (s, 1H), 5.09 (s, 1H), 3.26 (s, 2H), 2.87 (t, J=11.7 Hz, 2H), 2.48 (dd, J=4.0, 1.0 Hz, 6H), 1.96 (d, J=19.4 Hz, 4H). ¹⁹F NMR (282 MHz, DMSO-d₆) δ−132.15.

### Example 134: Synthesis of Compound 462

### Synthesis of Intermediate C98

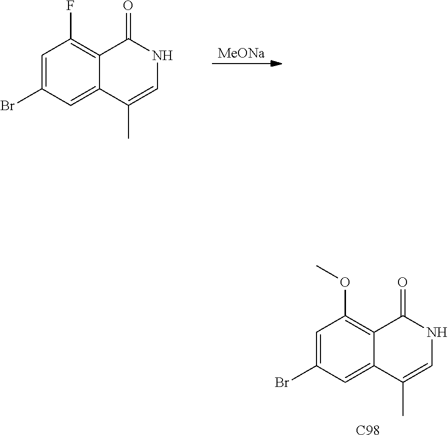

A mixture of 6-bromo-8-fluoro-4-methyl-2H-isoquinolin-1-one (200 mg, 0.781 mmol, 1 equiv) and sodium methoxide (210 mg, 3.905 mmol, 5 equiv) in THF (2 mL) was stirred for 3 h at 50° C. under nitrogen atmosphere, then cooled to room temperature. The reaction mixture was quenched with water (50 mL) and extracted with ethyl acetate (2×50 mL). The organic layers were combined, washed with brine (2×20 mL), dried over anhydrous Na₂SO₄, and filtered. After filtration, the filtrate was concentrated under reduced pressure to give a residue. The residue was purified by silica gel column chromatography, eluted with PE/EA (1:1) to afford 6-bromo-8-methoxy-4-methyl-2H-isoquinolin-1-one (150 mg, 72%) as a solid. LCMS (ES, m/z): 268 [M+H]⁺.

### Synthesis of Intermediate C99

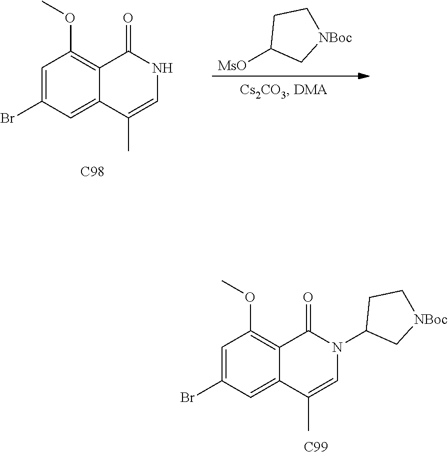 [SMILES: COc1cc(Br)cc2c(C)c[nH]c(=O)c12 ; CC(C)(C)OC(=O)N1CCC(OS(C)(=O)=O)C1 ; "C98" ; COc1cc(Br)cc2c(C)cn(C3CCN(C(=O)OC(C)(C)C)C3)c(=O)c12 ; "C99"]

To a stirred mixture of 6-bromo-8-methoxy-4-methyl-2H-isoquinolin-1-one (1 g, 3.73 mmol, 1 equiv) and tert-butyl 3-(methanesulfonyloxy)pyrrolidine-1-carboxylate (1.98 g, 7.46 mmol, 2 equiv) in DMA (30 mL) was added Cs₂CO₃ (3.65 g, 11.19 mmol, 3 equiv) in portions at room temperature under nitrogen atmosphere. The resulting mixture was stirred for 3 h at 110° C., then cooled to room temperature. The reaction mixture was quenched with water (50 mL) and extracted with ethyl acetate (2×50 mL). The organic layers were combined, washed with brine (2×30 mL), dried over anhydrous Na₂SO₄, and filtered. After filtration, the filtrate was concentrated under reduced pressure to give a residue. The residue was purified by silica gel column chromatography, eluted with PE/EA (5:1) to afford tert-butyl 3-(6-bromo-8-methoxy-4-methyl-1-oxoisoquinolin-2-yl)pyrrolidine-1-carboxylate (0.6 g, 37%) as a solid. LCMS (ES, m/z): 437 [M+H]⁺.

### Synthesis of Intermediate C100

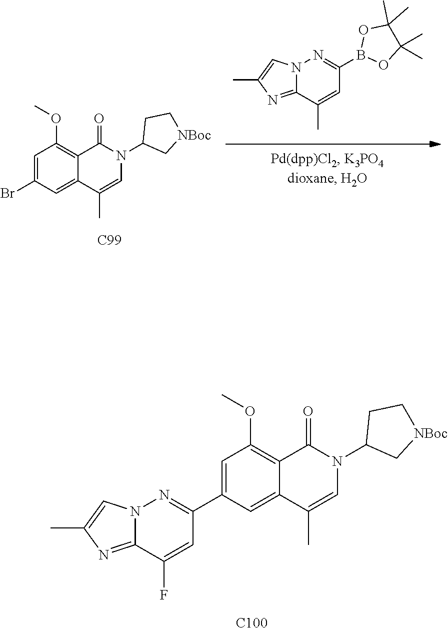

To a stirred mixture of tert-butyl 3-(6-bromo-8-methoxy-4-methyl-1-oxoisoquinolin-2-yl)pyrrolidine-1-carboxylate (500 mg, 1.143 mmol, 1 equiv), 2,8-dimethyl-6-(4,4,5,5-tetramethyl-1,3,2-dioxaborolan-2-yl)imidazo[1,2-b]pyridazine (374 mg, 1.372 mmol, 1.2 equiv), and K₃PO₄ (485.36 mg, 2.286 mmol, 2 equiv) in dioxane (10 mL) was added H₂O (1 mL) and Pd(dppf)Cl₂ (83 mg, 0.114 mmol, 0.1 equiv) in portions at room temperature under nitrogen atmosphere. The resulting mixture was stirred for 2 h at 80° C., then cooled to room temperature. The reaction mixture was quenched with water (50 mL) and extracted with ethyl acetate (2×100 mL). The organic layers were combined, washed with brine (3×30 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 3-(6-{2,8-dimethylimidazo[1,2-b]pyridazin-6-yl}-8-methoxy-4-methyl-1-oxoisoquinolin-2-yl)pyrrolidine-1-carboxylate (350 mg, 61%) as a solid. LCMS (ES, m/z): 504 [M+H]⁺.

### Synthesis of Compound 462

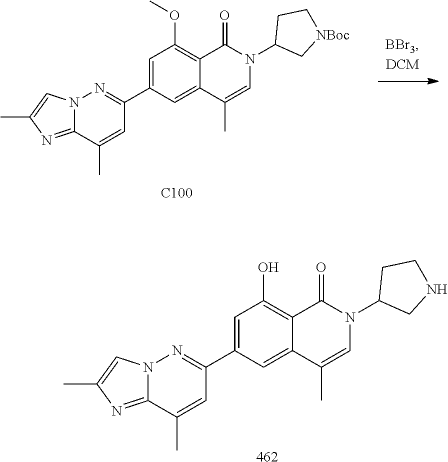 [SMILES: COc1cc(-c2cc(C)c3nc(C)cn3n2)cc2c(C)cn(C3CCN(C(=O)OC(C)(C)C)C3)c(=O)c12 ; "C100" ; Cc1cn2nc(-c3cc(O)c4c(=O)n(C5CCNC5)cc(C)c4c3)cc(C)c2n1 ; "462"]

A mixture of tert-butyl 3-(6-{2,8-dimethylimidazo[1,2-b]pyridazin-6-yl}-8-methoxy-4-methyl-1-oxoisoquinolin-2-yl)pyrrolidine-1-carboxylate (200 mg, 0.397 mmol, 1 equiv) and BBr₃ (2 mL) in DCM (20 mL) was stirred for 2 h at room temperature. The resulting mixture was concentrated under reduced pressure to give a residue. The residue was purified by Prep-HPLC (Condition 5, Gradient 2) to afford 8-hydroxy-4-methyl-6-{2-methylimidazo[1,2-b]pyridazin-6-yl}-2-(pyrrolidin-3-yl) isoquinolin-1-one (11 mg, 7%) as an oil. LCMS (ES, m/z): 390 [M+H]⁺. ¹H NMR (300 MHz, DMSO-d₆) 13.26 (s, 1H), 9.16 (s, 1H), 9.06 (s, 1H), 8.37-8.30 (m, 1H), 8.14 (s, 1H), 7.73 (d, J=1.6 Hz, 1H), 7.57 (d, J=1.5 Hz, 1H), 7.54-7.47 (m, 1H), 5.44-5.30 (m, 1H), 3.62 (d, J=11.2 Hz, 4H), 3.32 (s, 1H), 2.71-2.62 (m, 3H), 2.39-2.22 (m, 4H). ¹⁹F NMR (282 MHz, DMSO-d6) δ−74.40.

### Example 135: Synthesis of Compounds 441, 451 and 452

### Synthesis of Intermediate C101

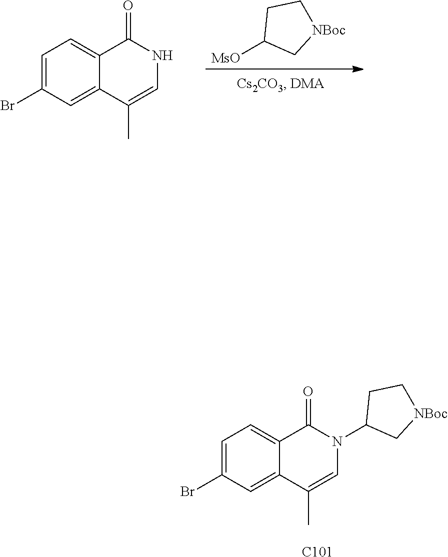 [SMILES: Cc1c[nH]c(=O)c2ccc(Br)cc12 ; CC(C)(C)OC(=O)N1CCC(OS(C)(=O)=O)C1 ; Cc1cn(C2CCN(C(=O)OC(C)(C)C)C2)c(=O)c2ccc(Br)cc12 ; "C101"]

To a stirred solution of 6-bromo-4-methyl-2H-isoquinolin-1-one (2.3 g, 9.66 mmol, 1 equiv) and tert-butyl 3-(methanesulfonyloxy)pyrrolidine-1-carboxylate (5.1 g, 19.32 mmol, 2 equiv) in dimethylacetamide (20 mL) was added CS₂CO₃ (9.4 g, 28.98 mmol, 3 equiv) at room temperature. The resulting mixture was stirred for 2 hr at 110° C. The mixture was allowed to cool down to room temperature. The reaction was quenched by the addition of water (50 mL) at room temperature. The resulting mixture was extracted with ethyl acetate (2×50 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EA (1:1) to afford tert-butyl 3-(6-bromo-4-methyl-1-oxoisoquinolin-2-yl)pyrrolidine-1-carboxylate (C101, 1.5 g, 38%) as a solid. LCMS (ES, m/z): 407 [M+H]⁺.

### Synthesis of Intermediate C102

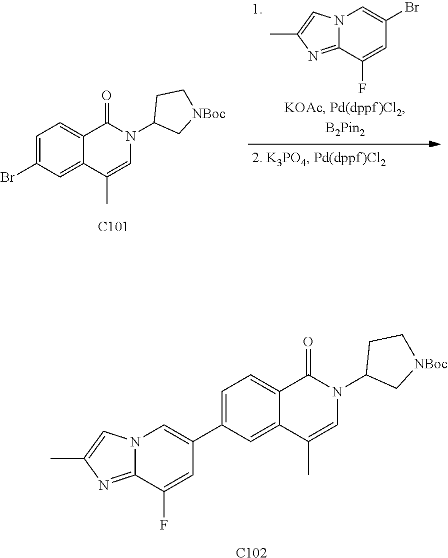 [SMILES: Cc1cn2cc(Br)cc(F)c2n1 ; Cc1cn(C2CCN(C(=O)OC(C)(C)C)C2)c(=O)c2ccc(Br)cc12 ; "C101" ; Cc1cn2cc(-c3ccc4c(=O)n(C5CCN(C(=O)OC(C)(C)C)C5)cc(C)c4c3)cc(F)c2n1 ; "C102"]

To a stirred solution of 6-bromo-8-fluoro-2-methylimidazo[1,2-a]pyridine (214 mg, 0.735 mmol, 1.50 equiv), B₂Pin₂ (250 mg, 0.98 mmol, 2 equiv) in dioxane (5 mL) was added potassium acetate (144 mg, 1.470 mmol, 3.00 equiv), Pd(dppf)Cl₂ (38 mg, 0.049 mmol, 0.10 equiv) at 25° C. under a nitrogen atmosphere. The resulting mixture was stirred for 3 hr at 80° C., then cooled to 25° C. To the reaction mixture was added tert-butyl 3-(6-bromo-4-methyl-1-oxoisoquinolin-2-yl)pyrrolidine-1-carboxylate (200 mg, 0.491 mmol, 1 equiv), K₃PO₄ (208 mg, 0.982 mmol, 2 equiv), H₂O (2 mL), and Pd(dppf)Cl₂ (36 mg, 0.049 mmol, 0.1 equiv) at room temperature under nitrogen atmosphere. The resulting mixture was stirred for 2 hr at 80° C., then cooled to room temperature. The resulting mixture was diluted with water (20 mL) and extracted with ethyl acetate (2×20 mL). The organic layers were combined, washed with brine (2×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 3-(6-{8-fluoro-2-methylimidazo[1,2-a]pyridin-6-yl}-4-methyl-1-oxoisoquinolin-2-yl)pyrrolidine-1-carboxylate (C102, 120 mg, 51%) as a solid. LCMS (ES, m/z): 478 [M+H]⁺.

### Synthesis of Compound 441

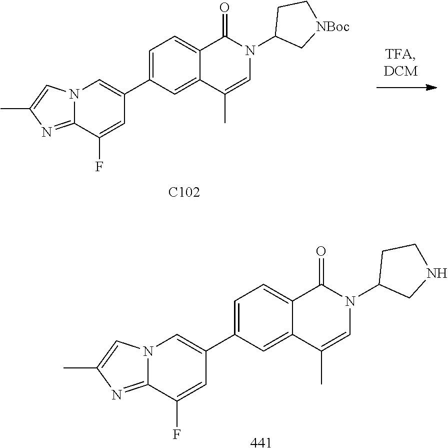 [SMILES: Cc1cn2cc(-c3ccc4c(=O)n(C5CCN(C(=O)OC(C)(C)C)C5)cc(C)c4c3)cc(F)c2n1 ; "C102" ; Cc1cn2cc(-c3ccc4c(=O)n(C5CCNC5)cc(C)c4c3)cc(F)c2n1 ; "441"]

To a stirred solution of tert-butyl 3-(6-{8-fluoro-2-methylimidazo[1,2-a]pyridin-6-yl}-4-methyl-1-oxoisoquinolin-2-yl)pyrrolidine-1-carboxylate (100 mg, 0.21 mmol, 1 equiv) in dichloromethane (5 mL) was added trifluoroacetic acid (119 mg, 1.05 mmol, 5 equiv) at 0° C. The resulting mixture was stirred for 2 hr at room temperature. The resulting mixture was concentrated under reduced pressure to give a residue. The residue was purified by reverse flash chromatography (Condition 2, Gradient 9) to afford 6-{8-fluoro-2-methylimidazo[1,2-a]pyridin-6-yl}-4-methyl-2-(pyrrolidin-3-yl)isoquinolin-1-one (Compound 441, 60 mg, 75%) as a solid. LCMS (ES, m/z): 377 [M+H]^{+ 1}H NMR (300 MHz, DMSO-d₆) δ 8.97 (d, J=1.5 Hz, 1H), 8.35 (dd, J=8.5, 4.6 Hz, 1H), 7.96-7.83 (m, 3H), 7.72 (dd, J=12.7, 1.5 Hz, 1H), 7.47-7.41 (m, 1H), 5.38 (p, J=8.6 Hz, 1H) 3.14 (dd, J=11.4, 7.7 Hz, 2H), 2.93-2.78 (m, 2H), 2.39 (dd, J=8.4, 1.0 Hz, 6H), 2.23 (dd, J=15.2, 6.8 Hz, 1H), 1.79 (dt, J=13.3, 6.6 Hz, 1H).

### Purification of Compounds 451 and 452

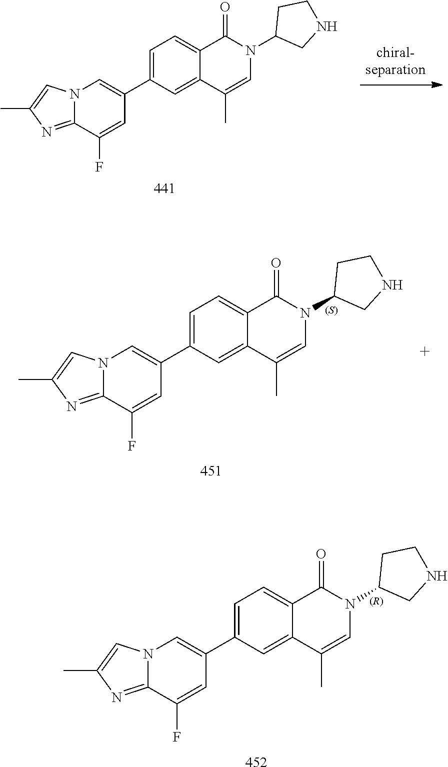

6-(8-fluoro-2-methylimidazo[1,2-a]pyridin-6-yl)-4-methyl-2-(pyrrolidin-3-yl)isoquinolin-1(2H)-one (50 mg) was purified by preparative chiral HPLC (Condition 7, Gradient 1) to afford
(S)-6-(8-fluoro-2-methylimidazo[1,2-a]pyridin-6-yl)-4-methyl-2-(pyrrolidin-3-yl)isoquinolin-1(2H)-one (Compound 451, 8 mg, 16%) as a solid and
(R)-6-(8-fluoro-2-methylimidazo[1,2-a]pyridin-6-yl)-4-methyl-2-(pyrrolidin-3-yl)isoquinolin-1(2H)-one (Compound 452, 10 mg, 20%) as a solid.

| Compound | |
|---|---|
| No. | Analysis Data |
| 451 | LCMS (ES, m/z): 377 [M + H]⁺ |
| | ¹H NMR (400 MHz, Methanol-d₄) δ 8.74 (d, J = 1.5 Hz, 1H), 8.48 |
| | (d, J = 8.4 Hz, 1H), 7.98 (d, J = 1.8 Hz, 1H), 7.88 (dd, J = 8.5, 1.8 |
| | Hz, 1H), 7.84-7.78 (m, 1H), 7.58 (dd, J = 11.9, 1.4 Hz, 1H), 7.35 (d, |
| | J = 1.4 Hz, 1H), 5.38 (p, J = 8.6 Hz, 1H), 3.38 (dd, J = 12.3, 8.2 Hz, |
| | 3H), 3.18 (dd, J = 12.3, 5.0 Hz, 1H), 3.07 (dt, J = 11.5, 7.8 Hz, 1H), |
| | 2.47 (dd, J = 9.3, 1.0 Hz, 7H), 2.13 (dt, J = 13.6, 7.1 Hz, 1H).. |
| 452 | LCMS (ES, m/z): 377 [M + H]⁺ |
| | ¹H NMR (400 MHz, Methanol-d₄) δ 8.72 (d, J = 1.5 Hz, 1H), 8.46 |
| | (d, J = 8.4 Hz, 1H), 7.96 (d, J = 1.8 Hz, 1H), 7.86 (dd, J = 8.4, 1.9 |
| | Hz, 1H), 7.79 (d, J = 2.9 Hz, 1H), 7.56 (dd, J = 11.9, 1.4 Hz, 1H), |
| | 7.33 (s, 1H), 5.37 (t, J = 14.2 Hz, 1H), 3.35 (d, J = 12.2 Hz, 3H), |
| | 3.17-3.09 (m, 1H), 3.03 (dt, J = 11.4, 7.7 Hz, 1H), 2.45 (dd, J = 8.9, |
| | 1.0 Hz, 7H), 2.37 (s, 1H), 2.08 (dq, J = 14.0, 7.6 Hz, 1H) |

### Example 136: Synthesis of Compound 362

### Synthesis of Intermediate C103

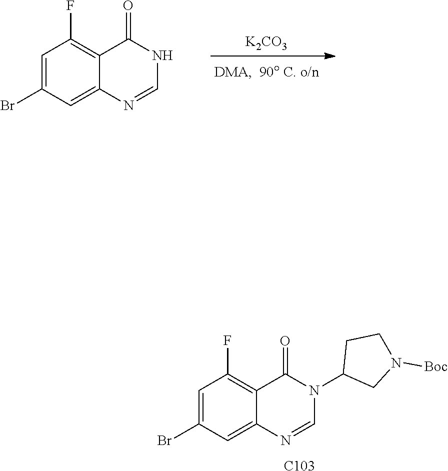

A mixture of 7-bromo-5-fluoro-3H-quinazolin-4-one (1.0 g, 4.115 mmol, 1.0 equiv), dimethylacetamide (10 mL), tert-butyl 3-(methanesulfonyloxy)pyrrolidine-1-carboxylate (1.31 g, 4.938 mmol, 1.2 equiv), and K₂CO₃ (1.71 g, 12.345 mmol, 3.0 equiv) was stirred overnight at 90° C. The resulting mixture was filtered, the filter cake was washed with dichloromethane (3×20 mL). The filtrate was concentrated under reduced pressure to give a residue. The residue was purified by reverse flash chromatography (Condition 2, Gradient 9) to afford tert-butyl 3-(7-bromo-5-fluoro-4-oxoquinazolin-3-yl)pyrrolidine-1-carboxylate (400 mg, 23%) as a solid. LCMS (ES, m/z): 412 [M+H]⁺.

### Synthesis of Intermediate C104

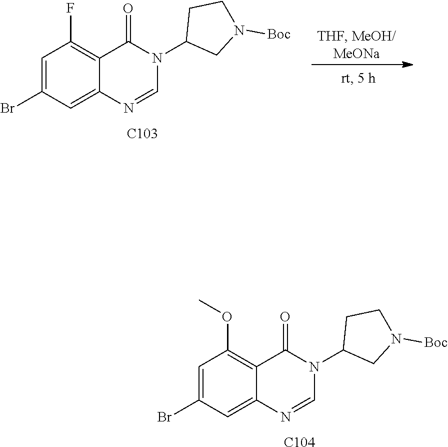 [SMILES: CC(C)(C)OC(=O)N1CCC(n2cnc3cc(Br)cc(F)c3c2=O)C1 ; "THF, MeOH/MeONa" ; "C103" ; COc1cc(Br)cc2ncn(C3CCN(C(=O)OC(C)(C)C)C3)c(=O)c12 ; "C104"]

A mixture of tert-butyl 3-(7-bromo-5-fluoro-4-oxoquinazolin-3-yl)pyrrolidine-1-carboxylate (400 mg, 0.970 mmol, 1.0 equiv), tetrahydrofuran (5 mL) and sodium methoxide (78.63 mg, 1.455 mmol, 1.5 equiv) was stirred for 5 hr at room temperature, diluted with water (10 mL) and extracted with CH₂Cl₂ (3×10 mL). The organic layers were combined, dried over anhydrous Na₂SO₄, and filtered. After filtration, the filtrate was concentrated under reduced pressure to afford tert-butyl 3-(7-bromo-5-methoxy-4-oxoquinazolin-3-yl)pyrrolidine-1-carboxylate (408 mg, 99%) as a solid. LCMS (ES, m/z): 424 [M+H]⁺.

### Synthesis of Intermediate C05

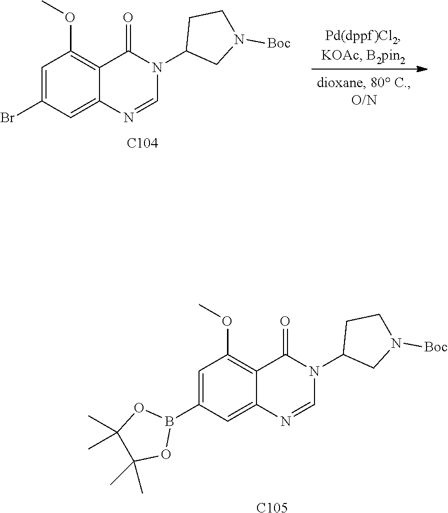 [SMILES: COc1cc(Br)cc2ncn(C3CCN(C(=O)OC(C)(C)C)C3)c(=O)c12 ; "Pd(dppf)Cl2, KOAc, B2pin2" ; "C104" ; COc1cc(B2OC(C)(C)C(C)(C)O2)cc2ncn(C3CCN(C(=O)OC(C)(C)C)C3)c(=O)c12 ; "C105"]

A mixture of tert-butyl 3-(7-bromo-5-methoxy-4-oxoquinazolin-3-yl)pyrrolidine-1-carboxylate (70 mg, 0.165 mmol, 1.0 equiv), 4,4,5,5-tetramethyl-2-(tetramethyl-1,3,2-dioxaborolan-2-yl)-1,3,2-dioxaborolane (50.27 mg, 0.198 mmol, 1.2 equiv), potassium acetate (48.57 mg, 0.495 mmol, 3.0 equiv), dioxane (1 mL) and Pd(dppf)Cl₂ (12.07 mg, 0.017 mmol, 0.1 equiv) was stirred overnight at 80° C. under nitrogen atmosphere. LCMS (ES, m/z): 472 [M+H]⁺.

### Synthesis of Intermediate C106

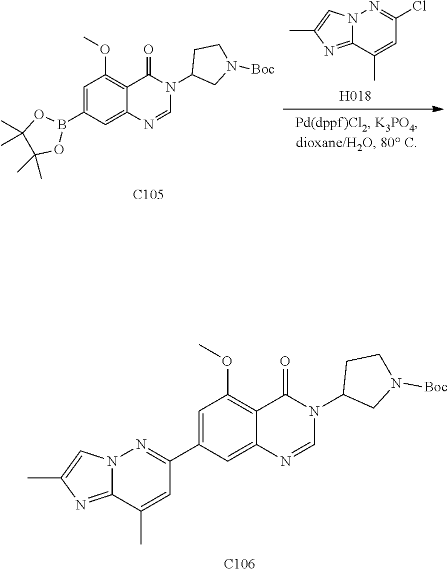

To the stirred mixture of C105 was added water (0.2 mL), 6-chloro-2,8-dimethylimidazo[1,2-b]pyridazine (61.65 mg, 0.340 mmol, 2.0 equiv), K₃PO₄ (36.03 mg, 0.170 mmol, 1.0 equiv) and Pd(dppf)Cl₂ (12.42 mg, 0.017 mmol, 0.1 equiv) at room temperature under nitrogen atmosphere. The resulting mixture was stirred overnight at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 3-(7-{2,8-dimethylimidazo[1,2-b]pyridazin-6-yl}-5-methoxy-4-oxoquinazolin-3-yl)pyrrolidine-1-carboxylate (C106, 70 mg, 84%) as an oil. LCMS (ES, m/z): 491 [M+H]⁺.

### Synthesis of Compound 362

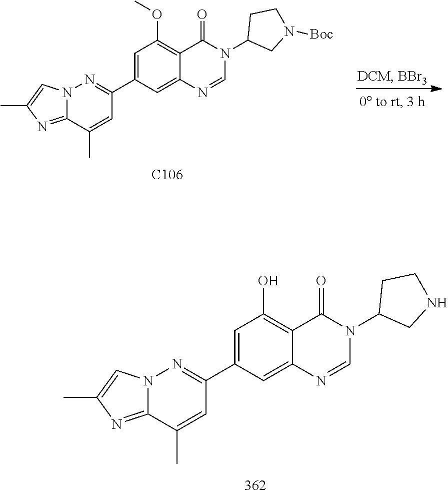 [SMILES: COc1cc(-c2cc(C)c3nc(C)cn3n2)cc2ncn(C3CCN(C(=O)OC(C)(C)C)C3)c(=O)c12 ; "C106" ; Cc1cn2nc(-c3cc(O)c4c(=O)n(C5CCNC5)cnc4c3)cc(C)c2n1 ; "362"]

A mixture of tert-butyl 3-(7-{2,8-dimethylimidazo[1,2-b]pyridazin-6-yl}-5-methoxy-4-oxoquinazolin-3-yl)pyrrolidine-1-carboxylate (50 mg, 0.102 mmol, 1.0 equiv), dichloromethane (1 mL) and BBr₃ (255.34 mg, 1.020 mmol, 10.0 equiv) was stirred for 3 hr at room temperature. The reaction was quenched with methanol (5 mL) at 0° C., then concentrated under reduced pressure to give a residue. The residue was purified by reverse phase flash (Condition 5, Gradient 4) to afford 7-{2,8-dimethylimidazo[1,2-b]pyridazin-6-yl}-5-hydroxy-3-(pyrrolidin-3-yl)quinazolin-4-one as an oil (Compound 362). LCMS (ES, m/z): 377 [M+H]+H NMR (300 MHz, Methanol-d₄) δ 8.30 (d, J=7.4 Hz, 2H), 8.18 (s, 1H), 7.90 (s, 1H), 7.69 (s, 1H), 5.09-4.97 (m, 1H), 3.90 (s, 2H), 3.74 (s, 1H), 3.40 (s, 2H), 2.79 (s, 3H), 2.73 (s, 2H), 2.65 (s, 3H).

### Example 137: Synthesis of Compound 442, 456, and 457

### Synthesis of Intermediate C107

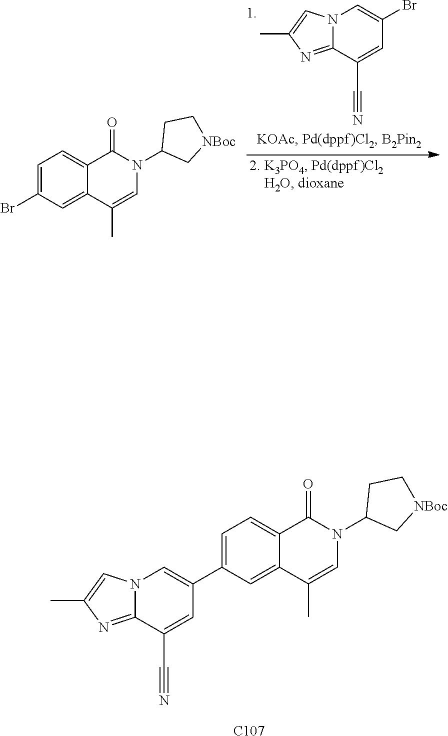 [SMILES: Cc1cn(C2CCN(C(=O)OC(C)(C)C)C2)c(=O)c2ccc(Br)cc12 ; Cc1cn2cc(Br)cc(C#N)c2n1 ; Cc1cn2cc(-c3ccc4c(=O)n(C5CCN(C(=O)OC(C)(C)C)C5)cc(C)c4c3)cc(C#N)c2n1 ; "C107"]

To a stirred solution of 6-bromo-2-methylimidazo[1,2-a]pyridine-8-carbonitrile (130 mg, 0.552 mmol, 1.50 equiv), B₂Pin₂ (187 mg, 0.736 mmol, 2.0 equiv) in dioxane (5 mL) was added and potassium acetate (108 mg, 1.104 mmol, 3.00 equiv) and Pd(dppf)Cl₂ (27 mg, 0.037 mmol, 0.10 equiv) at 25° C. under nitrogen atmosphere. The resulting mixture was stirred for 3 hr at 80° C. under nitrogen atmosphere, then cooled to 25° C. To the reaction mixture were added tert-butyl 3-(6-bromo-4-methyl-1-oxoisoquinolin-2-yl)pyrrolidine-1-carboxylate (150 mg, 0.368 mmol, 1.00 equiv), K₃PO₄ (156 mg, 0.735 mmol, 2.00 equiv), H₂O (1 mL) and Pd(dppf)Cl₂ (27 mg, 0.037 mmol, 0.10 equiv) at room temperature under nitrogen atmosphere. The resulting mixture was stirred for an additional 2 hr at 80° C. under nitrogen atmosphere, then cooled to room temperature. The reaction mixture was quenched with water (30 mL) and extracted with ethyl acetate (2×50 mL). The organic layers were combined, washed with brine (2×20 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 3-(6-{8-cyano-2-methylimidazo[1,2-a]pyridin-6-yl}-4-methyl-1-oxoisoquinolin-2-yl)pyrrolidine-1-carboxylate (C107, 110 mg, 61%) as a solid. LCMS (ES, m/z): 484[M+H]⁺.

### Synthesis of Compound 442

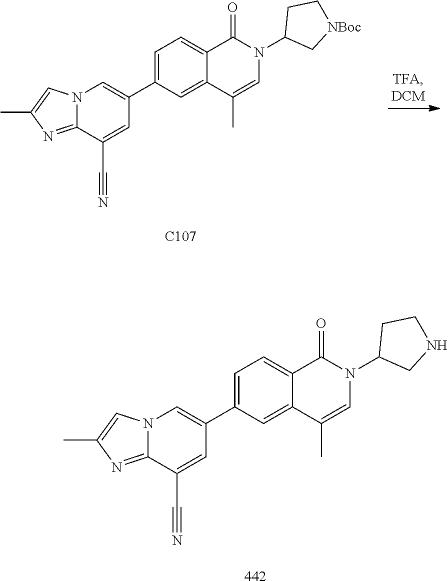 [SMILES: Cc1cn2cc(-c3ccc4c(=O)n(C5CCN(C(=O)OC(C)(C)C)C5)cc(C)c4c3)cc(C#N)c2n1 ; "C107" ; Cc1cn2cc(-c3ccc4c(=O)n(C5CCNC5)cc(C)c4c3)cc(C#N)c2n1 ; "442"]

A solution of tert-butyl 3-(6-{8-cyano-2-methylimidazo[1,2-a]pyridin-6-yl}-4-methyl-1-oxoisoquinolin-2-yl)pyrrolidine-1-carboxylate (100 mg, 0.207 mmol, 1 equiv) and trifluoroacetic acid (117 mg, 1.035 mmol, 5 equiv) in dichloromethane (5 mL) was stirred for 1 hr at room temperature. The reaction mixture was concentrated under reduced pressure to give a residue. The residue was purified by reverse flash chromatography (Condition 6, Gradient 1) to afford 2-methyl-6-[4-methyl-1-oxo-2-(pyrrolidin-3-yl) isoquinolin-6-yl]imidazo[1,2-a]pyridine-8-carbonitrile (60 mg, 75%) as a solid. LCMS (ES, m/z):384[M+H]^{+ 1}H NMR (300 MHz, DMSO-d₆) δ 9.39 (d, J=1.9 Hz, 1H), 8.54 (d, J=1.8 Hz, 1H), 8.36 (d, J=8.4 Hz, 1H), 8.01 (s, 1H), 7.92 (d, J=5.7 Hz, 2H), 7.45 (s, 1H), 5.38 (s, 1H), 3.17 (s, 2H), 2.91 (d, J=12.2 Hz, 2H), 2.41 (d, J=17.3 Hz, 6H), 2.09 (dd, J=14.1, 7.4 Hz, 1H), 1.78 (dd, J=14.1, 7.4 Hz, 1H).

### Purification of Compounds 456 and 457

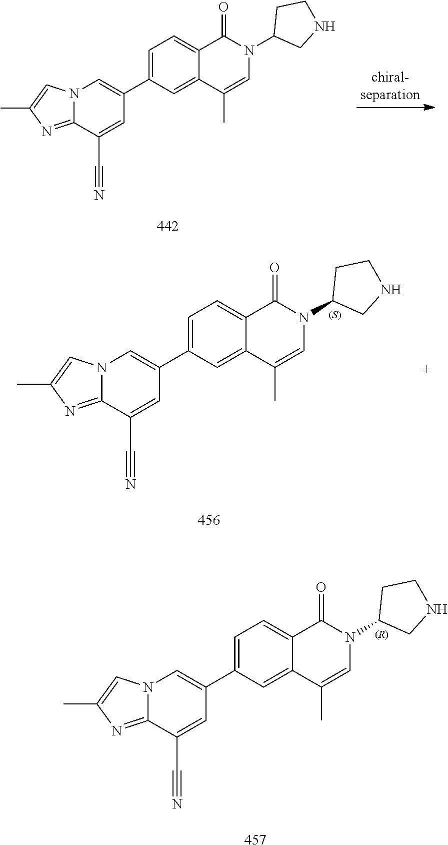

2-methyl-6-(4-methyl-1-oxo-2-(pyrrolidin-3-yl)-1,2-dihydroisoquinolin-6-yl)imidazo[1,2-a]pyridine-8-carbonitrile (Compound 442, 57 mg) was purified by Preparative Chiral-HPLC (Condition 4, Gradient 2) to afford (S)-2-methyl-6-(4-methyl-1-oxo-2-(pyrrolidin-3-yl)-1,2-dihydroisoquinolin-6-yl)imidazo[1,2-a]pyridine-8-carbonitrile (Compound 456, 15 mg, 26%) as a solid and (R)-2-methyl-6-(4-methyl-1-oxo-2-(pyrrolidin-3-yl)-1,2-dihydroisoquinolin-6-yl)imidazo[1,2-a]pyridine-8-carbonitrile (Compound 457, 16 mg, 28%) as a solid.

| Compound | |
|---|---|
| No. | Analysis Data |
| 456 | LCMS (ES, m/z): 384 [M + H]⁺ |
| | ¹H NMR (300 MHz, DMSO-d₆) δ 9.38 (d, J = 1.8 Hz, 1H), 8.54 (d, J = 1.8 |
| | Hz, 1H), 8.42-8.32 (m, 1H), 8.00 (d, J = 1.8 Hz, 1H), 7.97-7.87 (m, 2H), 7.45 |
| | (d, J = 1.3 Hz, 1H), 5.38 (s, 1H), 3.14 (d, J = 8.4 Hz, 2H), 2.90 (d, J = 8.7 Hz, |
| | 2H), 2.41 (dd, J = 17.5, 1.0 Hz, 6H), 2.11 (dd, J = 14.1, 7.4 Hz, 1H), 1.80 (dd, |
| | J = 14.1, 7.4 Hz, 1H). |
| 457 | LCMS (ES, m/z): 384 [M + H]⁺ |
| | ¹H NMR (300 MHz, DMSO-d₆) δ 9.38 (d, J = 1.8 Hz, 1H), 8.54 (d, J = 1.8 Hz, 1H), |
| | 8.42-8.32 (m, 1H), 8.00 (d, J = 1.8 Hz, 1H), 7.97-7.87 (m, 2H), 7.46 (d, J = 1.3 Hz, |
| | 1H), 5.38 (s, 1H), 3.14 (d, J = 8.4 Hz, 2H), 2.89 (d, J = 8.7 Hz, 2H), 2.39 (dd, J = |
| | 17.5, 1.0 Hz, 6H), 2.12 (dd, J = 14.1, 7.4 Hz, 1H), 1.78 (dd, J = 14.1, 7.4 Hz, 1H). |

### Experimental 138: Synthesis of Compound 501

### Synthesis of Intermediate C108

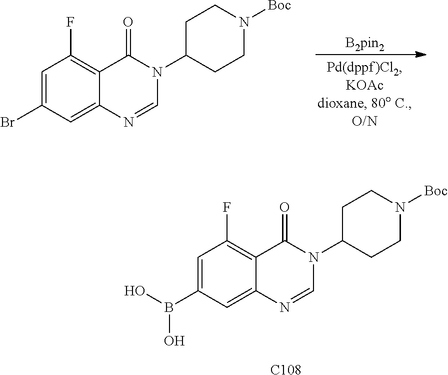 [SMILES: CC(C)(C)OC(=O)N1CCC(n2cnc3cc(Br)cc(F)c3c2=O)CC1 ; CC(C)(C)OC(=O)N1CCC(n2cnc3cc(B(O)O)cc(F)c3c2=O)CC1 ; "C108"]

To a stirred mixture of tert-butyl 4-(7-bromo-5-fluoro-4-oxoquinazolin-3-yl)piperidine-1-carboxylate (50 mg, 0.117 mmol, 1 equiv) and bis(pinacolato)diboron (44.68 mg, 0.176 mmol, 1.5 equiv) in dioxane (1 mL) was added potassium acetate (34.53 mg, 0.351 mmol, 3 equiv) and Pd(dppf)Cl₂ (8.58 mg, 0.012 mmol, 0.1 equiv) at room temperature under nitrogen atmosphere. The resulting mixture was stirred for 12 hr at 80° C. under nitrogen atmosphere. LCMS (ES, m/z): 392 [M+H]⁺.

### Synthesis of Intermediate C109

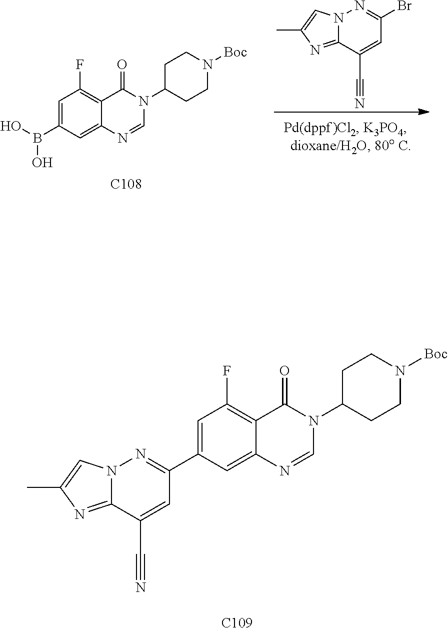 [SMILES: CC(C)(C)OC(=O)N1CCC(n2cnc3cc(B(O)O)cc(F)c3c2=O)CC1 ; Cc1cn2nc(Br)cc(C#N)c2n1 ; "C108" ; Cc1cn2nc(-c3cc(F)c4c(=O)n(C5CCN(C(=O)OC(C)(C)C)CC5)cnc4c3)cc(C#N)c2n1 ; "C109"]

To a stirred mixture of 6-chloro-2-methylimidazo[1,2-b]pyridazine-8-carbonitrile (24.39 mg, 0.127 mmol, 1.2 equiv) and C108 in dioxane (1 mL) and H₂O (0.4 mL) was added K₃PO₄ (22.40 mg, 0.106 mmol, 1 equiv) and Pd(dppf)Cl₂ (7.72 mg, 0.011 mmol, 0.1 equiv) at room temperature under nitrogen atmosphere. The resulting mixture was stirred for 12 hr at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 4-(7-{8-cyano-2-methylimidazo[1,2-b]pyridazin-6-yl}-5-fluoro-4-oxoquinazolin-3-yl)piperidine-1-carboxylate (C109, 50 mg, 94%) as a solid. LCMS (ES, m/z): 504 [M+H]⁺.

### Synthesis of Compound 501

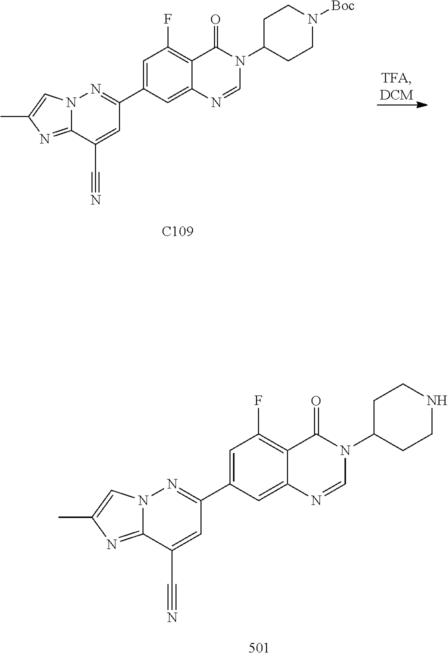 [SMILES: Cc1cn2nc(-c3cc(F)c4c(=O)n(C5CCN(C(=O)OC(C)(C)C)CC5)cnc4c3)cc(C#N)c2n1 ; "C109" ; Cc1cn2nc(-c3cc(F)c4c(=O)n(C5CCNCC5)cnc4c3)cc(C#N)c2n1]

To a stirred solution of tert-butyl 4-(7-{8-cyano-2-methylimidazo[1,2-b]pyridazin-6-yl}-5-fluoro-4-oxoquinazolin-3-yl)piperidine-1-carboxylate (50 mg, 0.099 mmol, 1 equiv) in dichloromethane (1 mL) was added trifluoroacetic acid (1 mL) at room temperature. The resulting mixture was stirred for 1 hr at room temperature. The resulting mixture was concentrated under reduced pressure to give a residue. The residue was purified by Prep-HPLC (Condition 6, Gradient 3) to afford tert-butyl 4-(7-{8-cyano-2-methylimidazo[1,2-b]pyridazin-6-yl}-5-fluoro-4-oxoquinazolin-3-yl)piperidine-1-carboxylate (Compound 501, 19.3 mg, 31%) as a solid. LCMS (ES, m/z): 404 [M+H]^{+ 1}H NMR (400 MHz, Methanol-d₄) δ 8.42 (s, 1H), 8.31 (s, 1H), 8.22 (dd, J=7.9, 1.4 Hz, 2H), 7.99 (dd, J=12.0, 1.7 Hz, 1H), 4.81 (s, 1H), 4.80 (s, OH), 3.65-3.57 (m, 2H), 3.24 (td, J=13.1, 2.9 Hz, 2H), 2.56 (d, J=0.9 Hz, 3H), 2.48 (td, J=13.0, 4.1 Hz, 2H), 2.24 (d, J=13.6 Hz, 2H).

### Example 139: Synthesis of Compound 502

### Synthesis of Intermediate C110

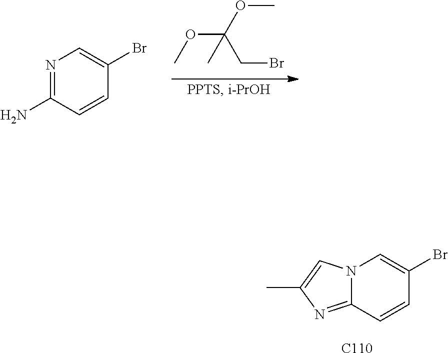

To a stirred solution of 5-bromopyridin-2-amine (3 g, 17.340 mmol, 1 equiv) in i-PrOH (60 mL) were added PPTS (0.44 g, 1.734 mmol, 0.1 equiv) and 1-bromo-2,2-dimethoxypropane (3.81 g, 20.808 mmol, 1.2 equiv) at room temperature. The resulting mixture was stirred for 2 hr at 80° C. The resulting mixture was concentrated under reduced pressure to give a residue. The residue was washed with ethyl acetate (3×50 mL) to afford 6-bromo-2-methylimidazo[1,2-a]pyridine (C110, 3 g, 81%) as a solid. LCMS (ES, m/z): 210 [M+H]⁺.

### Synthesis of Intermediate C111

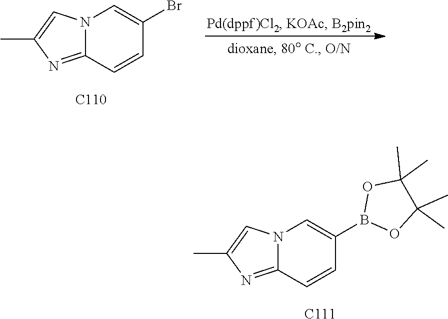

To a stirred mixture of 6-bromo-2-methylimidazo[1,2-a]pyridine (500 mg, 2.369 mmol, 1 equiv) and bis(pinacolato)diboron (902.37 mg, 3.554 mmol, 1.5 equiv) in dioxane (10 mL) was added potassium acetate (697.49 mg, 7.107 mmol, 3 equiv) and Pd(dppf)Cl₂ (173.34 mg, 0.237 mmol, 0.1 equiv) at room temperature under nitrogen atmosphere. The resulting mixture was stirred for overnight at 80° C. under nitrogen atmosphere. LCMS (ES, m/z): 259 [M+H]⁺.

### Synthesis of Intermediate C112

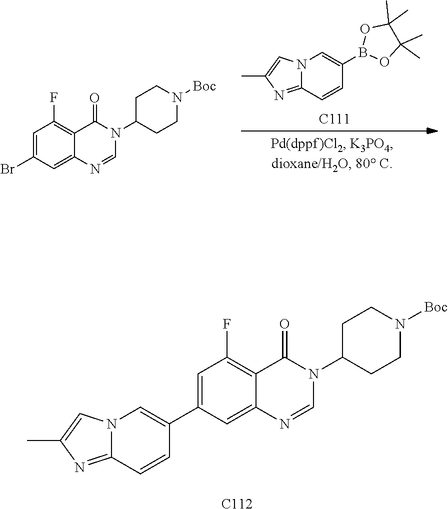

To a stirred mixture of tert-butyl 4-(7-bromo-5-fluoro-4-oxoquinazolin-3-yl)piperidine-1-carboxylate (150 mg, 0.352 mmol, 1 equiv) and C111 in dioxane (3 mL) and H₂O (0.6 mL) was added K₃PO₄ (224.07 mg, 1.056 mmol, 3 equiv) and Pd(dppf)Cl₂ (25.75 mg, 0.035 mmol, 0.1 equiv) at room temperature under nitrogen atmosphere. The resulting mixture was stirred overnight at 80° C. under nitrogen atmosphere, then concentrated under reduced pressure to give a residue. The residue was purified by silica gel column chromatography, eluted with CH₂Cl₂/MeOH (10:1) to afford tert-butyl 4-(5-fluoro-7-{2-methylimidazo[1,2-a]pyridin-6-yl}-4-oxoquinazolin-3-yl)piperidine-1-carboxylate (C112, 80 mg, 47%) as a solid. LCMS (ES, m/z): 478 [M+H]⁺.

### Synthesis of Intermediate C113

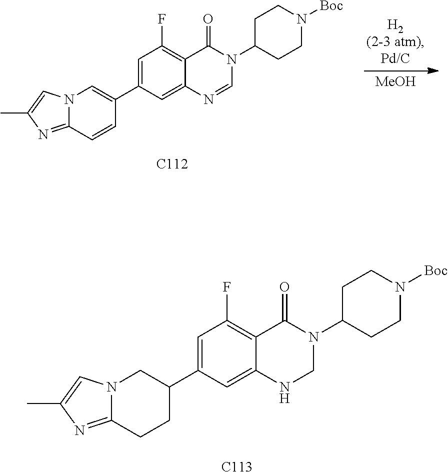 [SMILES: "MeOH" ; Cc1cn2cc(-c3cc(F)c4c(=O)n(C5CCN(C(=O)OC(C)(C)C)CC5)cnc4c3)ccc2n1 ; "C112" ; Cc1cn2c(n1)CCC(c1cc(F)c3c(c1)NCN(C1CCN(C(=O)OC(C)(C)C)CC1)C3=O)C2 ; "C113"]

To a solution of tert-butyl 4-(5-fluoro-7-{2-methylimidazo[1,2-a]pyridin-6-yl}-4-oxoquinazolin-3-yl)piperidine-1-carboxylate (80 mg, 0.168 mmol, 1 equiv) in methanol (5 mL) was added Pd/C (10%, 15 mg) under nitrogen atmosphere in a 50 mL round-bottom flask. The reaction mixture was hydrogenated at room temperature overnight under hydrogen atmosphere using a hydrogen balloon, filtered and concentrated under reduced pressure to afford tert-butyl 4-(5-fluoro-7-{2-methyl-5H,6H,7H,8H-imidazo[1,2-a]pyridin-6-yl}-4-oxo-1,2-dihydroquinazolin-3-yl)piperidine-1-carboxylate (C113, 60 mg, 74%) as an oil. LCMS (ES, m/z): 484 [M+H]⁺.

### Synthesis of Intermediate C114

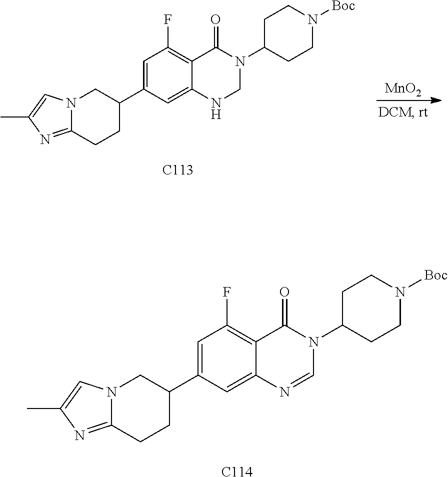 [SMILES: Cc1cn2c(n1)CCC(c1cc(F)c3c(c1)NCN(C1CCN(C(=O)OC(C)(C)C)CC1)C3=O)C2 ; "MnO2" ; "C113" ; Cc1cn2c(n1)CCC(c1cc(F)c3c(=O)n(C4CCN(C(=O)OC(C)(C)C)CC4)cnc3c1)C2 ; "C114"]

To a stirred solution of tert-butyl 4-(5-fluoro-7-{2-methyl-5H,6H,7H,8H-imidazo[1,2-a]pyridin-6-yl}-4-oxo-1,2-dihydroquinazolin-3-yl)piperidine-1-carboxylate (60 mg, 0.124 mmol, 1 equiv) in dichloromethane (5 mL) was added manganese dioxide (10 mg, 0.115 mmol, 0.93 equiv) at room temperature under nitrogen atmosphere. The resulting mixture was stirred for 12 hr at room temperature under nitrogen atmosphere, then filtered and the filtrate was concentrated under reduced pressure to afford tert-butyl 4-(5-fluoro-7-{2-methyl-5H,6H,7H,8H-imidazo[1,2-a]pyridin-6-yl}-4-oxoquinazolin-3-yl)piperidine-1-carboxylate (C114, 40 mg, 66%) as an oil. LCMS (ES, m/z): 482 [M+H]⁺.

### Synthesis of Compound 502

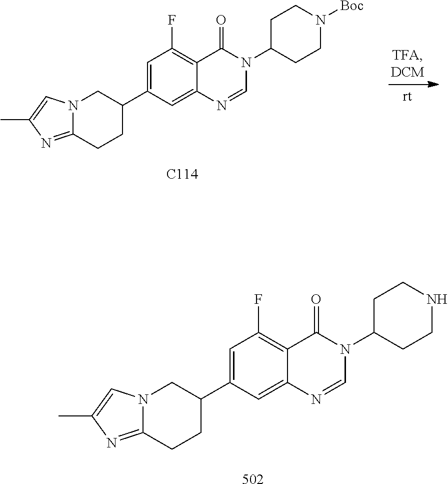 [SMILES: Cc1cn2c(n1)CCC(c1cc(F)c3c(=O)n(C4CCN(C(=O)OC(C)(C)C)CC4)cnc3c1)C2 ; "C114" ; Cc1cn2c(n1)CCC(c1cc(F)c3c(=O)n(C4CCNCC4)cnc3c1)C2 ; "502"]

To a stirred solution of tert-butyl 4-(5-fluoro-7-{2-methyl-5H,6H,7H,8H-imidazo[1,2-a]pyridin-6-yl}-4-oxoquinazolin-3-yl)piperidine-1-carboxylate (40 mg, 0.083 mmol, 1 equiv) in dichloromethane (1 mL) was added trifluoroacetic acid (1 mL, 13.463 mmol, 162.09 equiv) at room temperature. The resulting mixture was stirred for 2 hr at room temperature, then concentrated under reduced pressure to give a residue. The residue was purified by Prep-HPLC (Condition 6, Gradient 3) to afford 5-fluoro-7-{2-methyl-5H,6H,7H,8H-imidazo[1,2-a]pyridin-6-yl}-3-(piperidin-4-yl)quinazolin-4-one (Compound 502, 3.4 mg, 6%) as a solid. LCMS (ES, m/z): 382 [M+H]^{+ 1}H NMR (400 MHz, Methanol-d₄) δ 8.30 (s, 1H), 7.56-7.51 (m, 1H), 7.36 (dd, J=11.9, 1.7 Hz, 1H), 7.18 (d, J=1.3 Hz, 1H), 4.84-4.82 (m, 1H), 4.46 (dd, J=13.0, 5.2 Hz, 1H), 4.27-4.16 (m, 1H), 3.66-3.53 (m, 3H), 3.31-3.15 (m, 5H), 2.51 (qd, J=13.0, 4.1 Hz, 2H), 2.36 (d, J=1.1 Hz, 5H), 2.22 (d, J=13.5 Hz, 2H), 0.92 (t, J=6.7 Hz, 1H).

### Example 140: Synthesis of Compound 367

### Synthesis of Intermediate C115

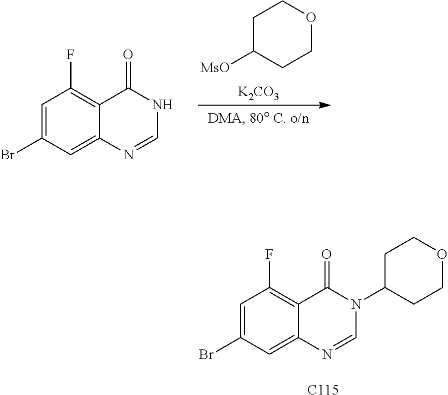

A mixture of 7-bromo-5-fluoro-3H-quinazolin-4-one (500 mg, 2.057 mmol, 1.0 equiv), dimethylacetamide (5 mL), oxan-4-yl methanesulfonate (444.92 mg, 2.468 mmol, 1.2 equiv) and K₂CO₃ (1137.33 mg, 8.228 mmol, 4.0 equiv) was stirred overnight at 80° C. The resulting mixture was filtered, the filter cake was washed with dichloromethane (3×20 mL). The filtrate was concentrated under reduced pressure to give a residue. The residue was purified by reverse flash chromatography (Condition 2, Gradient 5) to afford 7-bromo-5-fluoro-3-(oxan-4-yl)quinazolin-4-one (C115, 150 mg, 22%) as a solid. LCMS (ES, m/z): 327 [M+H]⁺.

### Synthesis of Intermediate C116

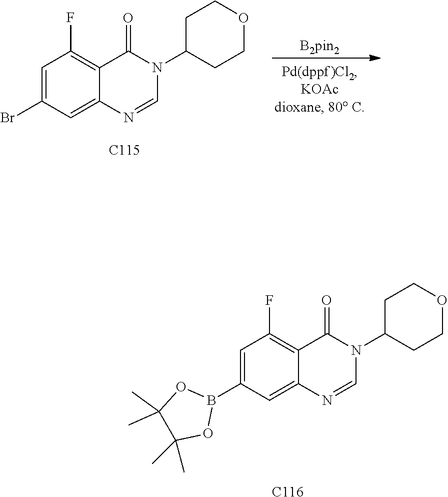 [SMILES: O=c1c2c(F)cc(Br)cc2ncn1C1CCOCC1 ; "C115" ; CC1(C)OB(c2cc(F)c3c(=O)n(C4CCOCC4)cnc3c2)OC1(C)C ; "C116"]

A mixture of 7-bromo-5-fluoro-3-(oxan-4-yl)quinazolin-4-one (70 mg, 0.214 mmol, 1.0 equiv), 4,4,5,5-tetramethyl-2-(tetramethyl-1,3,2-dioxaborolan-2-yl)-1,3,2-dioxaborolane (65.20 mg, 0.257 mmol, 1.2 equiv), potassium acetate (63.00 mg, 0.642 mmol, 3.0 equiv), dioxane (1 mL) and Pd(dppf)Cl₂ (15.66 mg, 0.021 mmol, 0.1 equiv) was stirred for 4 hr at 80° C. under nitrogen atmosphere. LCMS (ES, m/z): 375 [M+H]⁺.

### Synthesis of Compound 367

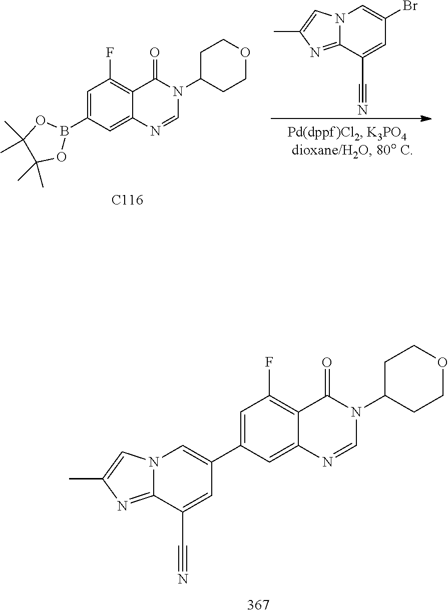

To the mixture of C116 was added water (0.2 mL), 6-bromo-2-methylimidazo[1,2-a]pyridine-8-carbonitrile (88.32 mg, 0.374 mmol, 2.0 equiv), K₃PO₄ (79.41 mg, 0.374 mmol, 2.0 equiv), and Pd(dppf)Cl₂ (13.69 mg, 0.019 mmol, 0.1 equiv) at room temperature under nitrogen atmosphere. The resulting mixture was stirred overnight at 80° C. under nitrogen atmosphere, then concentrated under reduced pressure to give a residue. The residue was purified by reverse phase flash (Condition 5, Gradient 4) to afford 6-[5-fluoro-3-(oxan-4-yl)-4-oxoquinazolin-7-yl]-2-methylimidazo[1,2-a]pyridine-8-carbonitrile (Compound 367, 11 mg, 14%) as a solid. LCMS (ES, m/z): 404 [M+H]^{+ 1}H NMR (400 MHz, DMSO-d₆) δ 9.47 (d, J=1.8 Hz, 1H), 8.57-8.50 (m, 2H), 7.96 (d, J=1.8 Hz, 1H), 7.90 (d, J=1.1 Hz, 1H), 7.81 (dd, J=12.5, 1.8 Hz, 1H), 4.91-4.80 (m, 1H), 4.02 (dd, J=11.3, 4.3 Hz, 2H), 3.50 (t, J=11.5 Hz, 2H), 2.44 (s, 3H), 2.12 (qd, J=12.3, 4.5 Hz, 2H), 1.81 (d, J=13.3 Hz, 2H).

### Example 141: Synthesis of Compounds 499 and 500

### Synthesis of Intermediate C117

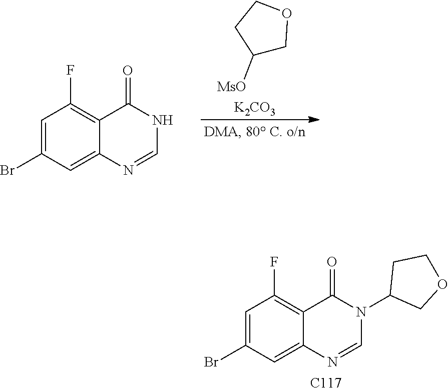

A mixture of 7-bromo-5-fluoro-3H-quinazolin-4-one (500 mg, 2.057 mmol, 1.0 equiv), dimethyl acetamide (5 mL), oxolan-3-yl methanesulfonate (410.29 mg, 2.468 mmol, 1.2 equiv) and K₂CO₃ (1137.33 mg, 8.228 mmol, 4.0 equiv) was stirred overnight at 80° C. The resulting mixture was filtered, the filter cake was washed with DCM (3×20 mL). The filtrate was concentrated under reduced pressure to give a residue. The residue was purified by reverse flash chromatography (Condition 3, Gradient 1)to afford 7-bromo-5-fluoro-3-(oxolan-3-yl)quinazolin-4-one (C117, 208 mg, 32%) as a solid. LCMS (ES, m/z): 313 [M+H]⁺.

### Synthesis of Intermediate C118

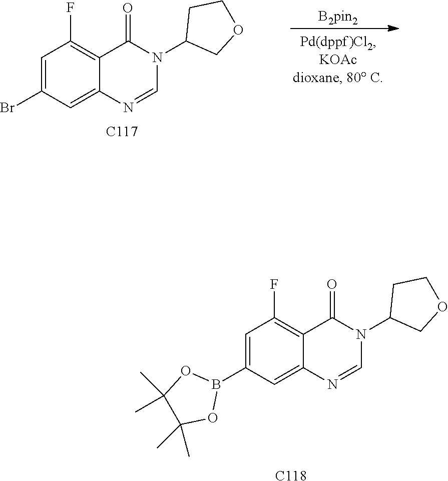 [SMILES: O=c1c2c(F)cc(Br)cc2ncn1C1CCOC1 ; "C117" ; CC1(C)OB(c2cc(F)c3c(=O)n(C4CCOC4)cnc3c2)OC1(C)C ; "C118"]

A mixture of 7-bromo-5-fluoro-3-(oxolan-3-yl)quinazolin-4-one (100 mg, 0.319 mmol, 1.0 equiv), 4,4,5,5-tetramethyl-2-(tetramethyl-1,3,2-dioxaborolan-2-yl)-1,3,2-dioxaborolane (97.32 mg, 0.383 mmol, 1.2 equiv), potassium acetate (94.03 mg, 0.957 mmol, 3.0 equiv), dioxane (1 mL) and Pd(dppf)Cl₂ (23.37 mg, 0.032 mmol, 0.1 equiv) was stirred for 4 hr at 80° C. under nitrogen atmosphere. LCMS (ES, m/z): 361 [M+H]⁺.

### Synthesis of Compound 366

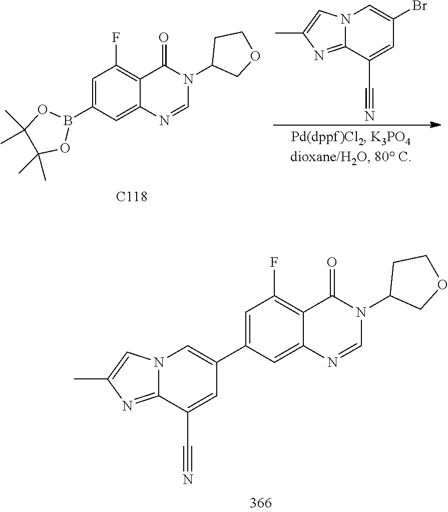

To the stirred mixture of C118 was added water (0.2 mL), 6-bromo-2-methylimidazo[1,2-a]pyridine-8-carbonitrile (150.74 mg, 0.638 mmol, 2.0 equiv), K₃PO₄ (135.54 mg, 0.638 mmol, 2.0 equiv) and Pd(dppf)Cl₂ (23.36 mg, 0.032 mmol, 0.1 equiv) at room temperature under nitrogen atmosphere. The resulting mixture was stirred overnight at 80° C. under nitrogen atmosphere, then concentrated under reduced pressure to give a residue. The residue was purified by reverse phase flash (Condition 5, Gradient 4) to afford 6-[5-fluoro-4-oxo-3-(oxolan-3-yl)quinazolin-7-yl]-2-methylimidazo[1,2-a]pyridine-8-carbonitrile (Compound 366, 35 mg, 28%) as a solid. LCMS (ES, m/z): 390 [M+H]^{+ 1}H NMR (300 MHz, DMSO-d₆) δ 9.48 (d, J=1.8 Hz, 1H), 8.54 (d, J=1.8 Hz, 1H), 8.30 (s, 1H), 7.97 (d, J=1.8 Hz, 1H), 7.90 (d, J=1.1 Hz, 1H), 7.82 (dd, J=12.6, 1.8 Hz, 1H), 5.33 (td, J=6.0, 3.1 Hz, 1H), 4.20-4.07 (m, 1H), 4.01 (dd, J=10.1, 2.9 Hz, 1H), 3.94-3.73 (m, 2H), 2.44 (d, J=0.9 Hz, 3H), 2.24-2.14 (m, 2H).

### Synthesis of Compounds 499 and 500

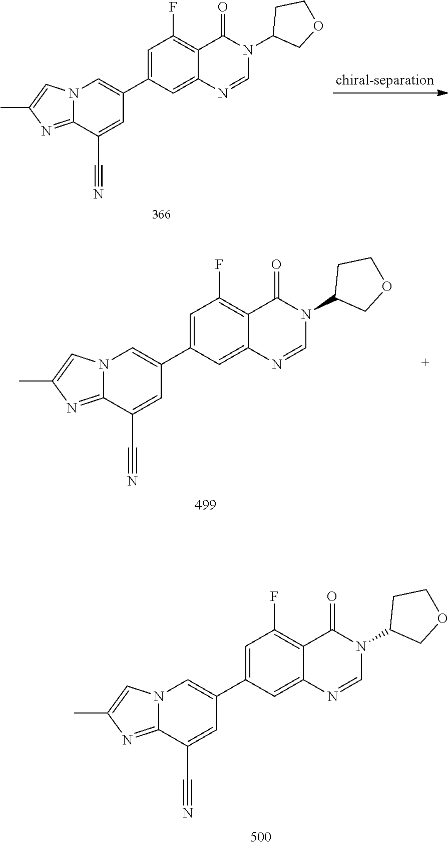

Compound 366 (30 mg) was purified by preparative chiral HPLC (Condition 8, Gradient 1) to afford (R)-6-(5-fluoro-4-oxo-3-(tetrahydrofuran-3-yl)-3,4-dihydroquinazolin-7-yl)-2-methylimidazo[1,2-a]pyridine-8-carbonitrile (Compound 499, 4 mg) as a solid and (S)-6-(5-fluoro-4-oxo-3-(tetrahydrofuran-3-yl)-3,4-dihydroquinazolin-7-yl)-2-methylimidazo[1,2-a]pyridine-8-carbonitrile (Compound 500, 4 mg) as a solid.

| Compound | |
|---|---|
| No. | Analysis Data |
| 499 | LCMS (ES, m/z): 390 [M + H]⁺ |
| | ¹H NMR (300 MHz, DMSO-d₆) δ 9.48 (d, J = 1.8 Hz, 1H), 8.53 (d, J = 1.8 |
| | Hz, 1H), 8.30 (s, 1H), 7.97 (s, 1H), 7.89 (d, J = 1.0 Hz, 1H), 7.82 (d, J = |
| | 12.7 Hz, 1H), 5.33 (s, 1H), 4.14 (q, J = 8.1 Hz, 1H), 4.01 (dd, J = 10.1, 2.9 |
| | Hz, 1H), 3.94-3.73 (m, 2H), 2.43 (d, J = 0.9 Hz, 3H), 2.21 (s, 2H). |
| 500 | LCMS (ES, m/z): 390 [M + H] ⁺ |
| | ¹H NMR (300 MHz, DMSO-d₆) δ 9.48 (d, J = 1.8 Hz, 1H), 8.53 (d, J = 1.8 |
| | Hz, 1H), 8.30 (s, 1H), 7.97 (s, 1H), 7.93-7.77 (m, 2H), 5.33 (s, 1H), 4.14 |
| | (q, J = 8.0 Hz, 1H), 4.06-3.96 (m, 1H), 3.94-3.73 (m, 2H), 2.50-2.40 |
| | (m, 3H), 2.20 (s, 2H). |

### Example 142: Synthesis of Compound 503

### Synthesis of Intermediate C119

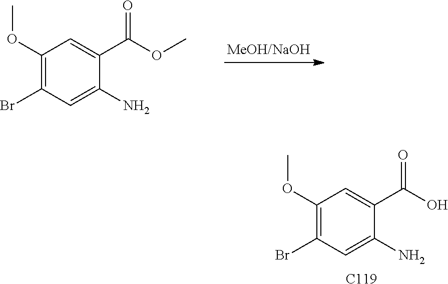

A mixture of methyl 2-amino-4-bromo-5-methoxybenzoate (3.0 g, 11.535 mmol, 1.0 equiv), methanol (30 mL), and water (10 mL) at room temperature. To the reaction mixture was added NaOH (1.38 g, 34.605 mmol, 3.0 equiv) in portions at 0° C. The resulting mixture was stirred overnight at room temperature. Methanol was removed under reduced pressure. The resulting mixture was acidified to pH 3 with concentrated HCl, then filtered, the filter cake was washed with petroleum ether (50 mL) to afford 2-amino-4-bromo-5-methoxybenzoic acid (C119, 2.1 g, 73%) as a solid. LCMS (ES, m/z): 246 [M+H]⁺.

### Synthesis of Intermediate C120

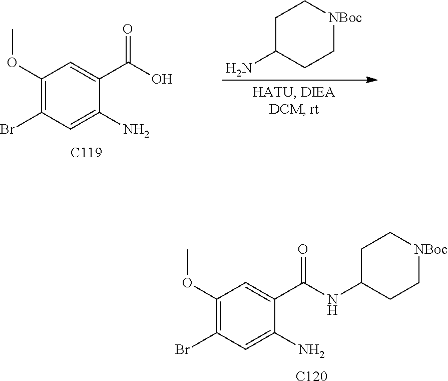 [SMILES: COc1cc(C(=O)O)c(N)cc1Br ; CC(C)(C)OC(=O)N1CCC(N)CC1 ; "C119" ; COc1cc(C(=O)NC2CCN(C(=O)OC(C)(C)C)CC2)c(N)cc1Br ; "C120"]

A mixture of 2-amino-4-bromo-5-methoxybenzoic acid (1.0 g, 4.064 mmol, 1.0 equiv), dichloromethane (10 mL), HATU (2.01 g, 5.283 mmol, 1.3 equiv), DIEA (1.58 g, 12.192 mmol, 3.0 equiv) and tert-butyl 4-aminopiperidine-1-carboxylate (0.98 g, 4.877 mmol, 1.2 equiv) was stirred for 6 hr at room temperature. The resulting mixture was concentrated under reduced pressure to give a residue. The residue was purified by silica gel column chromatography, eluted with PE/EA (3:1) to afford tert-butyl 4-(2-amino-4-bromo-5-methoxybenzamido)piperidine-1-carboxylate (C120, 1.3 g, 74%) as a solid. LCMS (ES, m/z): 428 [M+H]⁺.

### Synthesis of Intermediate C121

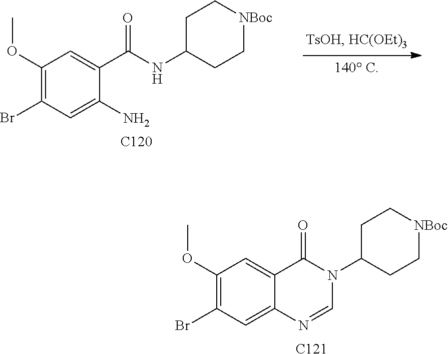 [SMILES: COc1cc(C(=O)NC2CCN(C(=O)OC(C)(C)C)CC2)c(N)cc1Br ; "C120" ; COc1cc2c(=O)n(C3CCN(C(=O)OC(C)(C)C)CC3)cnc2cc1Br ; "C121"]

A mixture of tert-butyl 4-(2-amino-4-bromo-5-methoxybenzamido)piperidine-1-carboxylate (1.3 g, 3.035 mmol, 1 equiv), triethyl orthoformate (15 mL) and TsOH (0.05 g, 0.304 mmol, 0.1 equiv) was stirred for 2 hr at 140° C. The resulting mixture was concentrated under reduced pressure to give a residue. The residue was purified by silica gel column chromatography, eluted with PE/EA (1:1) to afford tert-butyl 4-(7-bromo-6-methoxy-4-oxoquinazolin-3-yl)piperidine-1-carboxylate (C121, 0.4 g, 30%) as a solid. LCMS (ES, m/z): 438 [M+H]⁺

### Synthesis of Intermediate C122

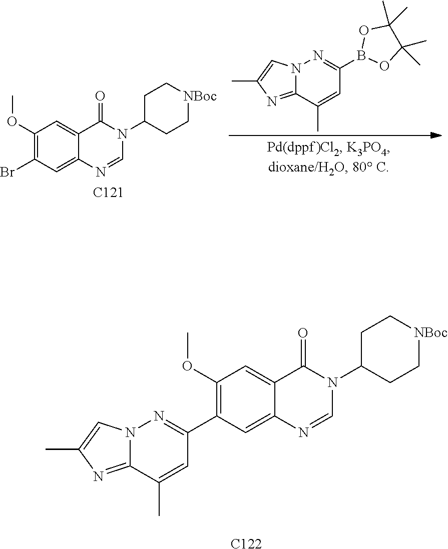

A mixture of tert-butyl 4-(7-bromo-6-methoxy-4-oxoquinazolin-3-yl)piperidine-1-carboxylate (300 mg, 0.684 mmol, 1.0 equiv), 2,8-dimethyl-6-(4,4,5,5-tetramethyl-1,3,2-dioxaborolan-2-yl)imidazo[1,2-b]pyridazine (224.33 mg, 0.821 mmol, 1.2 equiv), K₃PO₄ (290.56 mg, 1.368 mmol, 2.0 equiv), dioxane (3.00 mL), water (0.60 mL) and Pd(dppf)Cl₂ (50.08 mg, 0.068 mmol, 0.1 equiv) was stirred overnight at 80° C. under nitrogen atmosphere. The resulting mixture was concentrated under reduced pressure to give a residue. The residue was purified by silica gel column chromatography, eluted with CH₂Cl₂/MeOH (20:1) to afford tert-butyl 4-(7-{2,8-dimethylimidazo[1,2-b]pyridazin-6-yl}-6-methoxy-4-oxoquinazolin-3-yl)piperidine-1-carboxylate (C122, 200 mg, 57%) as a solid. LCMS (ES, m/z): 505 [M+H]⁺

### Synthesis of Compound 503

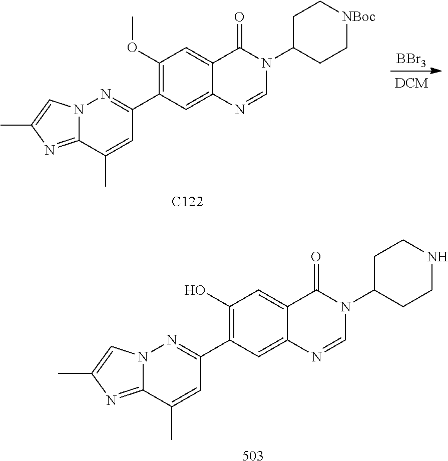

A mixture of tert-butyl 4-(7-{2,8-dimethylimidazo[1,2-b]pyridazin-6-yl}-6-methoxy-4-oxoquinazolin-3-yl)piperidine-1-carboxylate (100 mg, 0.198 mmol, 1.0 equiv), dichloromethane (1 mL) and boron tribromide (496.48 mg, 1.980 mmol, 10.0 equiv) was stirred for 16 hr at room temperature. The reaction mixture was quenched with methanol (5 mL) at 0° C., then concentrated under reduced pressure to give a residue. The residue product was purified by reverse phase flash (Condition 5, Gradient 4) to afford 7-{2,8-dimethylimidazo[1,2-b]pyridazin-6-yl}-6-hydroxy-3-(piperidin-4-yl)quinazolin-4-one (Compound 502, 9 mg, 9%) as a solid. LCMS (ES, m/z): 391 [M+H]^{+ 1}H NMR (400 MHz, Methanol-d₄) δ 8.25 (s, 1H), 8.19 (s, 1H), 8.12 (d, J=1.4 Hz, 1H), 8.06 (s, 1H), 7.77 (s, 1H), 3.62 (d, J=12.9 Hz, 2H), 3.31-3.19 (m, 2H), 2.77-2.73 (m, 3H), 2.63 (s, 3H), 2.61-2.54 (m, 1H), 2.50 (dd, J=13.2, 4.0 Hz, 1H), 2.22 (d, J=12.4 Hz, 2H).

### Example 143: Synthesis of Compound 432

### Synthesis of Intermediate C123

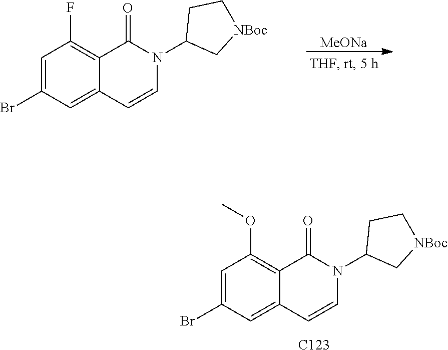 [SMILES: "MeONa" ; CC(C)(C)OC(=O)N1CCC(n2ccc3cc(Br)cc(F)c3c2=O)C1 ; COc1cc(Br)cc2ccn(C3CCN(C(=O)OC(C)(C)C)C3)c(=O)c12 ; "C123"]

A mixture of tert-butyl 3-(6-bromo-8-fluoro-1-oxoisoquinolin-2-yl)pyrrolidine-1-carboxylate (200 mg, 0.486 mmol, 1 equiv) and sodium methoxide (32 mg, 0.583 mmol, 1.2 equiv) in tetrahydrofuran (10 mL) was stirred for 5 h at room temperature. The reaction mixture was quenched with saturated NH₄Cl (aq.) (10 mL) at room temperature and extracted with ethyl acetate (3×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with PE/EA (1:10) to afford tert-butyl 3-(6-bromo-8-methoxy-1-oxoisoquinolin-2-yl)pyrrolidine-1-carboxylate (115 mg, 56%) as a solid. LCMS (ES, m/z): 423 [M+H]⁺.

### Synthesis of Intermediate C124

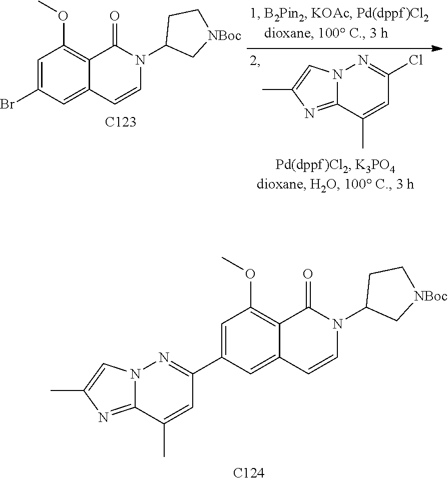 [SMILES: "C123" ; COc1cc(Br)cc2ccn(C3CCN(C(=O)OC(C)(C)C)C3)c(=O)c12 ; Cc1cn2nc(Cl)cc(C)c2n1 ; COc1cc(-c2cc(C)c3nc(C)cn3n2)cc2ccn(C3CCN(C(=O)OC(C)(C)C)C3)c(=O)c12 ; "C124"]

To a stirred mixture of tert-butyl 3-(6-bromo-8-methoxy-1-oxoisoquinolin-2-yl)pyrrolidine-1-carboxylate (95 mg, 0.224 mmol, 1 equiv) and bis(pinacolato)diboron (69 mg, 0.269 mmol, 1.2 equiv) in dioxane (5 mL) was added KOAc (44 mg, 0.448 mmol, 2 equiv) and Pd(dppf)Cl₂ (17 mg, 0.022 mmol, 0.1 equiv) in portions at room temperature under nitrogen atmosphere. The resulting mixture was stirred for 3 h at 100° C. under nitrogen atmosphere, then cooled to room temperature. To the resulting mixture was added 6-chloro-2,8-dimethylimidazo[1,2-b]pyridazine (41 mg, 0.224 mmol, 1 equiv), H₂O (1 mL), K₃PO₄ (95 mg, 0.448 mmol, 2 equiv), and Pd(dtbpf)Cl₂ (15 mg, 0.022 mmol, 0.1 equiv) in portions at room temperature under nitrogen atmosphere. The resulting mixture was stirred for an additional 3 h at 100° C. under nitrogen atmosphere, then cooled to room temperature. The resulting mixture was concentrated under reduced pressure to give a residue. The residue was purified by silica gel column chromatography, eluted with CH₂Cl₂/MeOH (10:1) to afford tert-butyl 3-(6-{2,8-dimethylimidazo[1,2-b]pyridazin-6-yl}-8-methoxy-1-oxoisoquinolin-2-yl)pyrrolidine-1-carboxylate (70 mg, 64%) as a solid. LCMS (ES, m/z): 490 [M+H]⁺.

### Synthesis of Compound 432

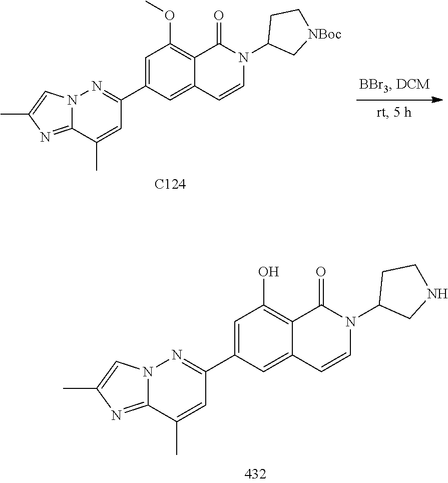 [SMILES: COc1cc(-c2cc(C)c3nc(C)cn3n2)cc2ccn(C3CCN(C(=O)OC(C)(C)C)C3)c(=O)c12 ; "C124" ; Cc1cn2nc(-c3cc(O)c4c(=O)n(C5CCNC5)ccc4c3)cc(C)c2n1]

To a stirred solution of tert-butyl 3-(6-{2,8-dimethylimidazo[1,2-b]pyridazin-6-yl}-8-methoxy-1-oxoisoquinolin-2-yl)pyrrolidine-1-carboxylate (35 mg, 0.071 mmol, 1 equiv) in DCM (5 mL) was added BBr₃ (90 mg, 0.355 mmol, 5 equiv) dropwise at room temperature. The resulting mixture was stirred for 5 h at room temperature, then concentrated under vacuum to give a residue. The residue was quenched with MeOH at room temperature. The resulting mixture was concentrated under vacuum to give a residue. The residue was purified by reverse flash chromatography (Condition 2, Gradient 13) to afford 6-{2,8-dimethylimidazo[1,2-b]pyridazin-6-yl}-8-hydroxy-2-(pyrrolidin-3-yl)isoquinolin-1-one (16.3 mg, 56%) as a solid. LCMS (ES, m/z): 376 [M+H]⁺. ¹H NMR (300 MHz, DMSO-d₆) δ 13.20 (s, 1H), 8.10 (s, 1H), 7.81-7.70 (m, 2H), 7.66 (d, J=7.6 Hz, 1H), 7.46 (d, J=1.6 Hz, 1H), 6.89 (d, J=7.6 Hz, 1H), 5.35 (s, 1H), 3.17 (dd, J=11.7, 7.5 Hz, 1H), 3.11 (s, 1H), 2.89 (ddd, J=23.4, 11.2, 5.8 Hz, 2H), 2.62 (s, 3H), 2.42 (s, 3H), 2.40-2.19 (m, 1H), 1.94-1.75 (m, 1H).

### Example 144: Synthesis of Compound 433

### Synthesis of Intermediate C125

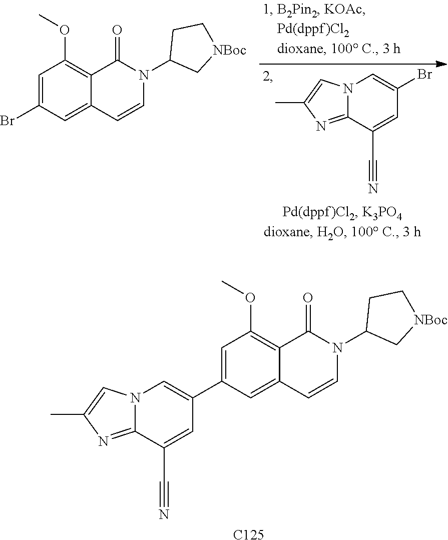 [SMILES: COc1cc(Br)cc2ccn(C3CCN(C(=O)OC(C)(C)C)C3)c(=O)c12 ; Cc1cn2cc(Br)cc(C#N)c2n1 ; COc1cc(-c2cc(C#N)c3nc(C)cn3c2)cc2ccn(C3CCN(C(=O)OC(C)(C)C)C3)c(=O)c12 ; "C125"]

To a stirred mixture of tert-butyl 3-(6-bromo-8-methoxy-1-oxoisoquinolin-2-yl)pyrrolidine-1-carboxylate (90 mg, 0.213 mmol, 1 equiv) and bis(pinacolato)diboron (65 mg, 0.256 mmol, 1.2 equiv) in dioxane (10 mL) was added KOAc (42 mg, 0.426 mmol, 2 equiv) and Pd(dppf)Cl₂ (16 mg, 0.021 mmol, 0.1 equiv) in portions at room temperature under nitrogen atmosphere. The resulting mixture was stirred for 3 h at 100° C. under nitrogen atmosphere, then cooled to room temperature. To the resulting mixture was added 6-chloro-2,8-dimethylimidazo[1,2-b]pyridazine (39 mg, 0.213 mmol, 1 equiv), H₂O (2 mL), K₃PO₄ (90 mg, 0.426 mmol, 2 equiv), and Pd(dtbpf)Cl₂ (14 mg, 0.021 mmol, 0.1 equiv) in portions at room temperature under nitrogen atmosphere, then stirred for an additional 3 h at 100° C. under nitrogen atmosphere. The resulting mixture was cooled to room temperature, then concentrated under reduced pressure to give a residue. The residue was purified by silica gel column chromatography, eluted with CH₂Cl₂/MeOH (10:1) to afford tert-butyl 3-(6-{8-cyano-2-methylimidazo[1,2-a]pyridin-6-yl}-8-methoxy-1-oxoisoquinolin-2-yl)pyrrolidine-1-carboxylate (30 mg, 28%) as a solid. LCMS (ES, m/z): 500 [M+H]⁺.

### Synthesis of Compound 433

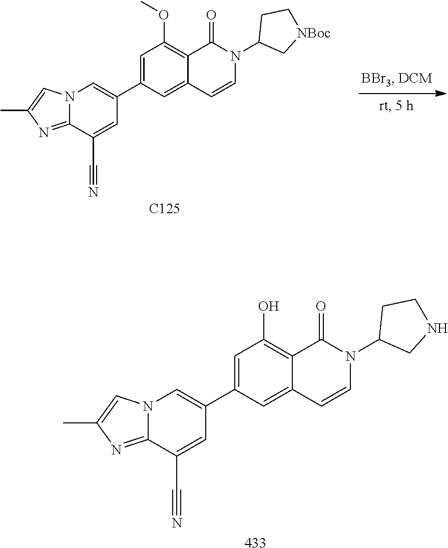 [SMILES: COc1cc(-c2cc(C#N)c3nc(C)cn3c2)cc2ccn(C3CCN(C(=O)OC(C)(C)C)C3)c(=O)c12 ; "C125" ; Cc1cn2cc(-c3cc(O)c4c(=O)n(C5CCNC5)ccc4c3)cc(C#N)c2n1 ; "433"]

To a stirred solution of tert-butyl 3-(6-{8-cyano-2-methylimidazo[1,2-a]pyridin-6-yl}-8-methoxy-1-oxoisoquinolin-2-yl)pyrrolidine-1-carboxylate (90 mg, 0.180 mmol, 1 equiv) in DCM (10 mL) was added BBr₃ (226 mg, 0.900 mmol, 5 equiv) dropwise at room temperature. The resulting mixture was stirred for 5 h at room temperature, then concentrated under reduced pressure to give a residue. The residue was quenched with methanol at room temperature. The resulting mixture was concentrated under vacuum to give a residue. The residue was purified by reverse flash chromatography (Condition 5, Gradient 1) to afford 6-(8-hydroxy-1-oxo-2-(pyrrolidin-3-yl)-1,2-dihydroisoquinolin-6-yl)-2-methylimidazo[1,2-a]pyridine-8-carbonitrile (7.1 mg, 10%) as a solid. LCMS (ES, m/z): 386 [M+H]⁺. ¹H NMR (300 MHz, DMSO-d₆) δ 9.28 (d, J=1.7 Hz, 1H), 8.39 (d, J=1.7 Hz, 1H), 7.91 (d, J=1.1 Hz, 1H), 7.55-7.46 (m, 2H), 7.26 (d, J=1.7 Hz, 1H), 6.82 (d, J=7.6 Hz, 1H), 5.23 (p, J=7.4 Hz, 1H), 3.59 (d, J=7.4 Hz, 3H), 3.35-3.19 (m, 1H), 2.42 (d, J=0.9 Hz, 4H), 2.39-2.26 (m, 1H).

### Example 145: Synthesis of Compound 434

### Synthesis of Intermediate C126

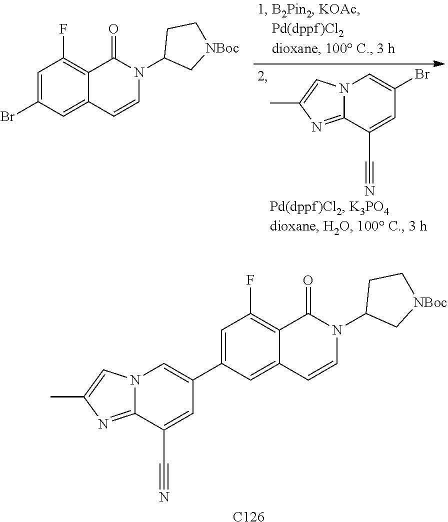 [SMILES: CC(C)(C)OC(=O)N1CCC(n2ccc3cc(Br)cc(F)c3c2=O)C1 ; Cc1cn2cc(Br)cc(C#N)c2n1 ; Cc1cn2cc(-c3cc(F)c4c(=O)n(C5CCN(C(=O)OC(C)(C)C)C5)ccc4c3)cc(C#N)c2n1 ; "C126"]

To a stirred mixture of tert-butyl 3-(6-bromo-8-fluoro-1-oxoisoquinolin-2-yl)pyrrolidine-1-carboxylate (90 mg, 0.219 mmol, 1 equiv) and bis(pinacolato)diboron (67 mg, 0.263 mmol, 1.2 equiv) in dioxane (10 mL) was added KOAc (43 mg, 0.438 mmol, 2 equiv) and Pd(dppf)Cl₂ (16 mg, 0.022 mmol, 0.1 equiv) in portions at room temperature under nitrogen atmosphere. The resulting mixture was stirred for 3 h at 100° C. under nitrogen atmosphere, then cooled to room temperature. To the resulting mixture was added 6-chloro-2,8-dimethylimidazo[1,2-b]pyridazine (40 mg, 0.219 mmol, 1 equiv), K₃PO₄ (140 mg, 0.657 mmol, 3 equiv), H₂O (2 mL) and Pd(dtbpf)Cl₂ (14 mg, 0.022 mmol, 0.1 equiv) in portions at room temperature. The resulting mixture was stirred for an additional 3 h at 100° C., then cooled to room temperature. The resulting mixture was concentrated under reduced pressure to give a residue. The residue was purified by silica gel column chromatography, eluted with CH₂Cl₂/MeOH (1:1) to afford tert-butyl 3-(6-{8-cyano-2-methylimidazo[1,2-a]pyridin-6-yl}-8-fluoro-1-oxoisoquinolin-2-yl)pyrrolidine-1-carboxylate (60 mg, 56%) as a solid. LCMS (ES, m/z): 488 [M+H]⁺.

### Synthesis of Compound 434

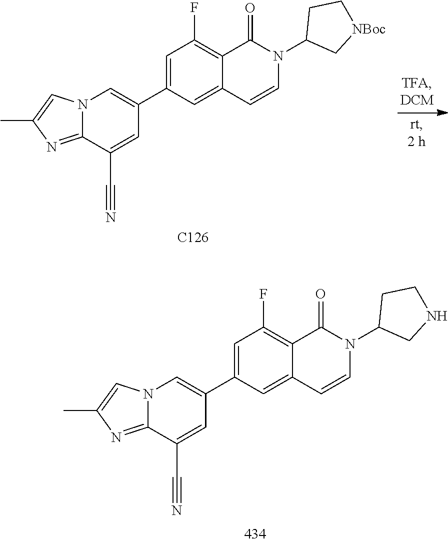 [SMILES: Cc1cn2cc(-c3cc(F)c4c(=O)n(C5CCN(C(=O)OC(C)(C)C)C5)ccc4c3)cc(C#N)c2n1 ; "C126" ; Cc1cn2cc(-c3cc(F)c4c(=O)n(C5CCNC5)ccc4c3)cc(C#N)c2n1]

A solution of tert-butyl 3-(6-{8-cyano-2-methylimidazo[1,2-a]pyridin-6-yl}-8-fluoro-1-oxoisoquinolin-2-yl)pyrrolidine-1-carboxylate (50 mg, 0.103 mmol, 1 equiv) in TFA (2 mL) and DCM (2 mL) was stirred for 2 h at room temperature. The resulting mixture was concentrated under vacuum to give a residue. The residue was basified to pH 8 with 7 M NH₃ in methanol. The resulting mixture was concentrated under vacuum to give a residue. The residue was purified by reverse flash chromatography (Condition 2, Gradient 8) to afford 6-[8-fluoro-1-oxo-2-(pyrrolidin-3-yl)isoquinolin-6-yl]-2-methylimidazo[1,2-a]pyridine-8-carbonitrile (20 mg, 50%) as a solid. LCMS (ES, m/z): 388 [M+H]⁺. ¹H NMR (400 MHz, DMSO-d₆) δ 9.39 (d, J=1.8 Hz, 1H), 8.45 (d, J=1.8 Hz, 1H), 7.94-7.87 (m, 2H), 7.73-7.63 (m, 2H), 6.67 (dd, J=7.6, 2.1 Hz, 1H), 5.32 (td, J=9.3, 8.7, 4.4 Hz, 1H), 3.19-3.04 (m, 2H), 2.91-2.80 (m, 2H), 2.43 (s, 3H), 2.22 (ddd, J=13.3, 8.7, 5.0 Hz, 1H), 1.74 (dq, J=13.2, 6.9 Hz, 1H).

### Example 146: Synthesis of Compound 443

### Synthesis of Intermediate C127

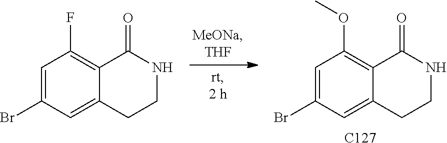

To a stirred solution of 6-bromo-8-fluoro-3,4-dihydro-2H-isoquinolin-1-one (1 g, 4.097 mmol, 1 equiv) in THF (10 mL) was added NaOMe (1.2 mL, 5.553 mmol, 1.36 equiv, 25%) dropwise at 0° C. The resulting mixture was stirred for 2 h at room temperature. The reaction mixture was quenched with NH₄Cl (aq.) at room temperature. A precipitate formed that was collected by filtration to afford 6-bromo-8-methoxy-3,4-dihydro-2H-isoquinolin-1-one (800 mg, 76%) as a solid. LCMS (ES, m/z): 256 [M+H]⁺.

### Synthesis of Intermediate C128

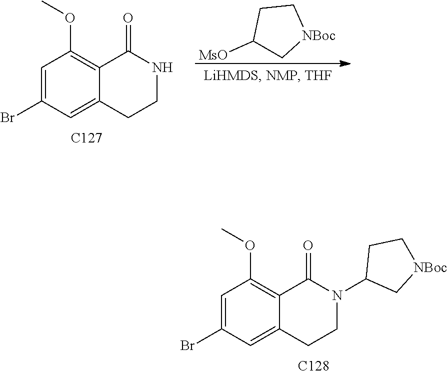 [SMILES: COc1cc(Br)cc2c1C(=O)NCC2 ; CC(C)(C)OC(=O)N1CCC(OS(C)(=O)=O)C1 ; "C127" ; COc1cc(Br)cc2c1C(=O)N(C1CCN(C(=O)OC(C)(C)C)C1)CC2 ; "C128"]

A solution of 6-bromo-8-methoxy-3,4-dihydro-2H-isoquinolin-1-one (500 mg, 1.952 mmol, 1 equiv) in NMP (5 mL) and THF (5 mL) was treated with tert-butyl 3-(methanesulfonyloxy) pyrrolidine-1-carboxylate (777 mg, 2.928 mmol, 1.5 equiv). The resulting mixture was stirred for 15 min at room temperature, followed by the addition of LiHMDS (5 mL, 59.762 mmol, 30.61 equiv) dropwise at room temperature. The resulting mixture was stirred for 3 h at 120° C., then concentrated under reduced pressure to give a residue. The residue was purified by reverse flash chromatography (Condition 1, Gradient 2) to afford tert-butyl 3-(6-bromo-8-methoxy-1-oxo-3,4-dihydroisoquinolin-2-yl)pyrrolidine-1-carboxylate (141 mg, 17%) as a solid. LCMS (ES, m/z): 425 [M+H]⁺.

### Synthesis of Intermediate C129

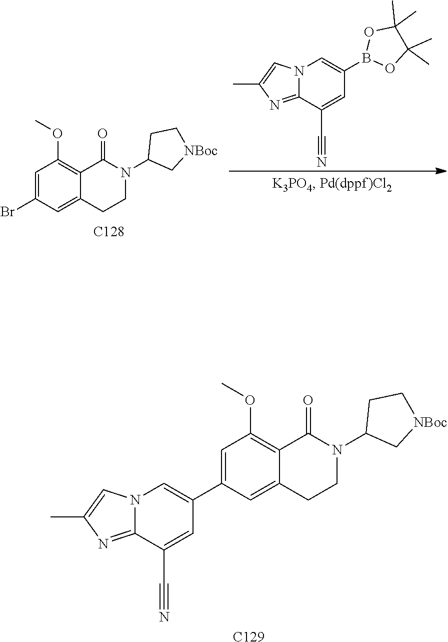

To a mixture of tert-butyl 3-(6-bromo-8-methoxy-1-oxo-3,4-dihydroisoquinolin-2-yl) pyrrolidine-1-carboxylate (200 mg, 0.470 mmol, 1 equiv) and 2-methyl-6-(4,4,5,5-tetramethyl-1,3,2-dioxaborolan-2-yl)imidazo[1,2-a]pyridine-8-carbonitrile (200 mg, 0.706 mmol, 1.50 equiv) in dioxane (4 mL) and H₂O (1 mL) was added K₃PO₄ (300 mg, 1.410 mmol, 3.0 equiv) and Pd(dppf)Cl₂CH₂Cl₂ (38 mg, 0.047 mmol, 0.1 equiv). After stirring for 1 h at 80° C. under a nitrogen atmosphere, the resulting mixture was concentrated under reduced pressure to give a residue. The residue was purified by reverse flash chromatography (Condition 1, Gradient 3) to afford tert-butyl 3-(6-{8-cyano-2-methylimidazo[1,2-a]pyridin-6-yl}-8-methoxy-1-oxo-3,4-dihydroisoquinolin-2-yl)pyrrolidine-1-carboxylate (170 mg, 72%) as a solid. LCMS (ES, m/z): 502 [M+H]⁺.

### Synthesis of Compound 443

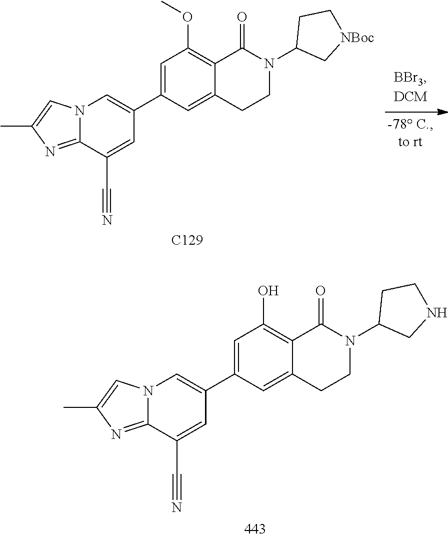 [SMILES: COc1cc(-c2cc(C#N)c3nc(C)cn3c2)cc2c1C(=O)N(C1CCN(C(=O)OC(C)(C)C)C1)CC2 ; "C129" ; Cc1cn2cc(-c3cc(O)c4c(c3)CCN(C3CCNC3)C4=O)cc(C#N)c2n1 ; "443"]

To a stirred solution of tert-butyl 3-(6-{8-cyano-2-methylimidazo[1,2-a]pyridin-6-yl}-8-methoxy-1-oxo-3,4-dihydroisoquinolin-2-yl)pyrrolidine-1-carboxylate (140 mg, 0.279 mmol, 1 equiv) in DCM (2 mL) was added BBr₃ (0.16 mL, 1.674 mmol, 6.0 equiv) at −78° C. under nitrogen atmosphere. The resulting mixture was stirred for 2 h at room temperature, then concentrated under reduced pressure to give a residue. The residue was quenched with H₂O (1 mL) at 0° C., then neutralized to pH 7 with NaHCO₃ (aq.). The resulting mixture was purified by reverse flash chromatography (Condition 5, Gradient 2) to afford 6-(8-hydroxy-1-oxo-2-(pyrrolidin-3-yl)-1,2,3,4-tetrahydroisoquinolin-6-yl)-2-methylimidazo[1,2-a]pyridine-8-carbonitrile (2 mg, 2%) as a solid. LCMS (ES, m/z): 388 [M+H]⁺. ¹H NMR (300 MHz, DMSO-d₆) δ 12.92 (s, 1H), 9.29 (s, 1H), 8.35 (s, 1H), 7.87 (s, 1H), 7.18 (s, 2H), 3.60 (s, 2H), 2.99 (dd, J=11.8, 7.3 Hz, 4H), 2.79 (s, 2H), 2.42 (s, 4H), 2.33-1.70 (m, 1H).

### Example 148: Synthesis of Compound 364

### Synthesis of Intermediate C135

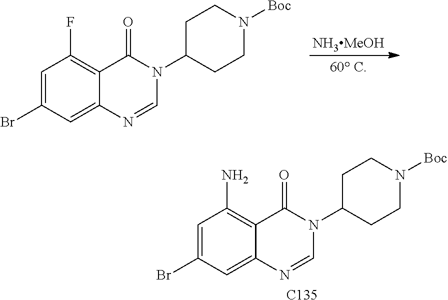 [SMILES: CC(C)(C)OC(=O)N1CCC(n2cnc3cc(Br)cc(F)c3c2=O)CC1 ; CC(C)(C)OC(=O)N1CCC(n2cnc3cc(Br)cc(N)c3c2=O)CC1 ; "C135" ; "NH3·MeOH"]

Tert-butyl 4-(7-bromo-5-fluoro-4-oxoquinazolin-3-yl)piperidine-1-carboxylate (200 mg, 0.469 mmol, 1 equiv) and methanol amine (6 mL) were combined at room temperature. The resulting mixture was stirred for 2 days at 60° C. The resulting mixture was concentrated under reduced pressure to afford tert-butyl 4-(5-amino-7-bromo-4-oxoquinazolin-3-yl)piperidine-1-carboxylate (102 mg, 51%) as a solid. LCMS (ES, m/z): 423 [M+H]⁺.

### Synthesis of Intermediate C136

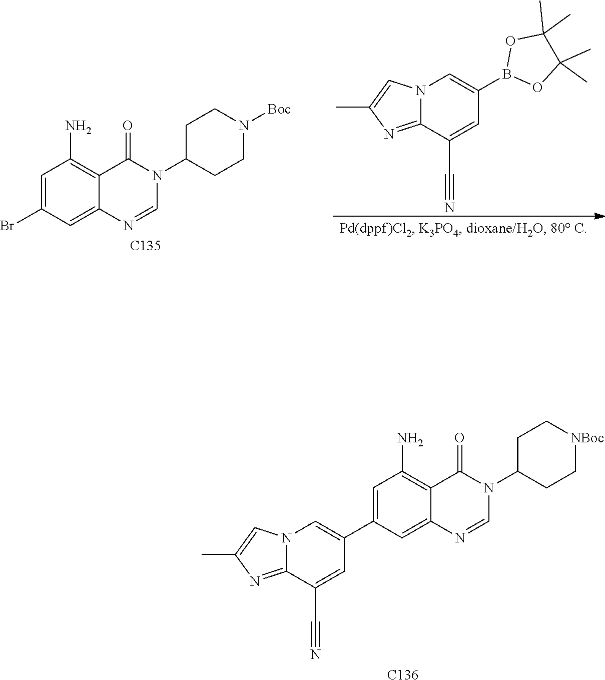

Tert-butyl 4-(5-amino-7-bromo-4-oxoquinazolin-3-yl)piperidine-1-carboxylate (100 mg, 0.236 mmol, 1.0 equiv), 2-methyl-6-(4,4,5,5-tetramethyl-1,3,2-dioxaborolan-2-yl)imidazo[1,2-a]pyridine-8-carbonitrile (80.26 mg, 0.283 mmol, 1.2 equiv), K₃PO₄ (100.29 mg, 0.472 mmol, 2.0 equiv), dioxane (1 mL), water (0.2 mL), and Pd(dppf)Cl₂ (17.29 mg, 0.024 mmol, 0.1 equiv) were combined at room temperature under nitrogen atmosphere. The resulting mixture was stirred overnight at 80° C. under nitrogen atmosphere. The resulting mixture was concentrated under reduced pressure to give a residue. The residue was purified by silica gel column chromatography, eluted with CH₂Cl₂/MeOH (20:1) to afford tert-butyl4-(5-amino-7-{8-cyano-2-methylimidazo[1,2-a]pyridin-6-yl}-4-oxoquinazolin-3-yl)piperidine-1-carboxylate (60 mg, 51%) as a solid. LCMS (ES, m/z): 500 [M+H]⁺.

### Synthesis of Compound 364

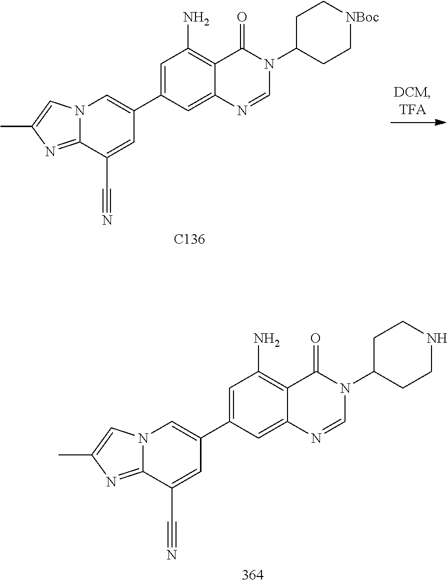 [SMILES: Cc1cn2cc(-c3cc(N)c4c(=O)n(C5CCN(C(=O)OC(C)(C)C)CC5)cnc4c3)cc(C#N)c2n1 ; "C136" ; Cc1cn2cc(-c3cc(N)c4c(=O)n(C5CCNCC5)cnc4c3)cc(C#N)c2n1 ; "364"]

A mixture of tert-butyl 4-(5-amino-7-{8-cyano-2-methylimidazo[1,2-a]pyridin-6-yl}-4-oxoquinazolin-3-yl)piperidine-1-carboxylate (50 mg, 0.100 mmol, 1 equiv), DCM (1 mL), and TFA (1 mL, 13.463 mmol, 134.52 equiv) was stirred for 1 h at room temperature. The resulting mixture was concentrated under reduced pressure to give a residue. The residue was purified by reverse phase flash (Condition 4, Gradient 1) to afford 6-[5-amino-4-oxo-3-(piperidin-4-yl)quinazolin-7-yl]-2-methylimidazo[1,2-a]pyridine-8-carbonitrile (10 mg, 25%) as a solid. LCMS (ES, m/z): 400 [M+H]. ¹H NMR (300 MHz, Methanol-d₄) δ 9.03 (d, J=1.7 Hz, 1H), 8.20-8.11 (m, 2H), 7.86 (s, 1H), 7.03 (d, J=1.8 Hz, 1H), 6.96 (d, J=1.8 Hz, 1H), 4.83 (ddd, J=12.1, 8.2, 3.9 Hz, 1H), 3.63 (d, J=13.1 Hz, 2H), 3.23 (d, J=13.5 Hz, 2H), 2.50 (s, 3H), 2.41 (d, J=13.6 Hz, 2H), 2.22 (d, J=13.7 Hz, 2H).

### Example 149: Synthesis of Compound 363

### Synthesis of Intermediate C137

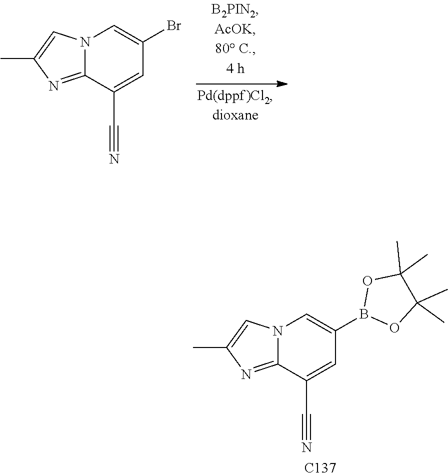

6-bromo-2-methylimidazo[1,2-a]pyridine-8-carbonitrile (60 mg, 0.254 mmol, 1.0 equiv), 4,4,5,5-tetramethyl-2-(tetramethyl-1,3,2-dioxaborolan-2-yl)-1,3,2-dioxaborolane (77.45 mg, 0.305 mmol, 1.2 equiv), AcOK (74.83 mg, 0.762 mmol, 3.0 equiv), and Pd(dppf)Cl₂ (18.60 mg, 0.025 mmol, 0.1 equiv) were combined at room temperature under nitrogen atmosphere. The resulting mixture was stirred for 4 h at 80° C. under nitrogen atmosphere. LCMS (ES, m/z): 202 [M+H]⁺.

### Synthesis of Intermediate C138

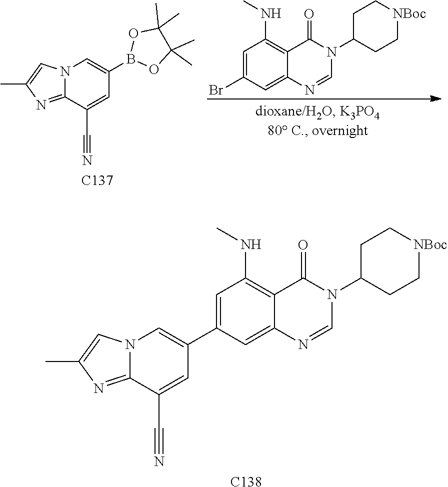

To C137 was added water (0.2 mL), tert-butyl 4-[7-bromo-5-(methylamino)-4-oxoquinazolin-3-yl]piperidine-1-carboxylate (108.12 mg, 0.247 mmol, 1.0 equiv), K₃PO₄ (52.48 mg, 0.247 mmol, 1.0 equiv), and Pd(dppf)Cl₂ (18.09 mg, 0.025 mmol, 0.1 equiv) at room temperature under nitrogen atmosphere. The resulting mixture was stirred overnight at 80° C. under nitrogen atmosphere. The resulting mixture was diluted with water (5 mL) and extracted with ethyl acetate (3×5 mL). The organic layers were combined, washed with brine (10 mL), dried over anhydrous Na₂SO₄, and filtered. The filtrated was concentrated under reduced pressure to give a residue. The residue was purified by silica gel column chromatography, eluted with MeOH/DCM (1:10) to afford tert-butyl 4-(7-{8-cyano-2-methylimidazo[1,2-a]pyridin-6-yl}-5-(methylamino)-4-oxoquinazolin-3-yl)piperidine-1-carboxylate (60 mg, 47%) as a solid. LCMS (ES, m/z): 514 [M+H]⁺.

### Synthesis of Compound 363

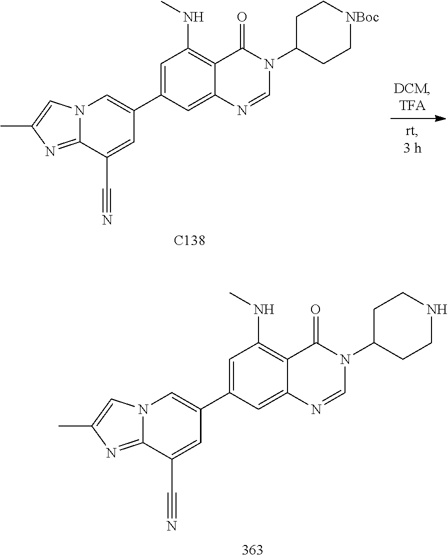 [SMILES: CNc1cc(-c2cc(C#N)c3nc(C)cn3c2)cc2ncn(C3CCN(C(=O)OC(C)(C)C)CC3)c(=O)c12 ; "C138" ; CNc1cc(-c2cc(C#N)c3nc(C)cn3c2)cc2ncn(C3CCNCC3)c(=O)c12]

Tert-butyl 4-(7-{8-cyano-2-methylimidazo[1,2-a]pyridin-6-yl}-5-(methylamino)-4-oxoquinazolin-3-yl)piperidine-1-carboxylate (50 mg, 0.097 mmol, 1 equiv), DCM (1 mL) and TFA (1 mL, 13.463 mmol, 138.29 equiv) were combined at room temperature. The resulting mixture was stirred for 3 h at room temperature. The resulting mixture was concentrated under reduced pressure to give a residue. The residue was purified by reverse phase flash (Condition 4, Gradient 1) to afford 2-methyl-6-[5-(methylamino)-4-oxo-3-(piperidin-4-yl)quinazolin-7-yl]imidazo[1,2-a]pyridine-8-carbonitrile (14 mg, 35%) as a solid. LCMS (ES, m/z): 414 [M+H]⁺. ¹H NMR (400 MHz, DMSO-d₆) δ 9.33 (d, J=1.8 Hz, 1H), 8.66 (q, J=5.0 Hz, 1H), 8.43 (d, J=1.7 Hz, 1H), 8.28 (s, 1H), 7.90 (s, 1H), 7.09 (d, J=1.7 Hz, 1H), 6.83 (d, J=1.7 Hz, 1H), 4.65 (ddd, J=12.1, 8.2, 3.9 Hz, 1H), 3.17 (d, J=13.2 Hz, 2H), 2.99 (d, J=5.0 Hz, 3H), 2.76-2.66 (m, 2H), 2.43 (s, 3H), 1.96 (qd, J=12.5, 4.2 Hz, 2H), 1.86-1.74 (m, 2H).

### Example 150: Synthesis of Compound 365

### Synthesis of Intermediate C139

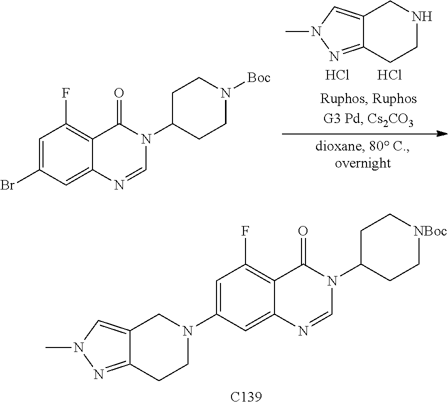

Tert-butyl 4-(7-bromo-5-fluoro-4-oxoquinazolin-3-yl)piperidine-1-carboxylate (50 mg, 0.117 mmol, 1 equiv), 2-methyl-4H,5H,6H,7H-pyrazolo[4,3-c]pyridine (17.70 mg, 0.129 mmol, 1.1 equiv), Cs₂CO₃ (114.65 mg, 0.351 mmol, 3.0 equiv), RuPhos (10.95 mg, 0.023 mmol, 0.2 equiv), dioxane (1 mL), and RuPhos Palladacycle Gen. 3 (9.81 mg, 0.012 mmol, 0.1 equiv) were combined at room temperature under nitrogen atmosphere. The resulting mixture was stirred overnight at 80° C. under nitrogen atmosphere. The resulting mixture was concentrated under reduced pressure to give a residue. The residue was purified by silica gel column chromatography, eluted with CH₂Cl₂/MeOH (20:1) to afford tert-butyl 4-(5-fluoro-7-{2-methyl-4H,6H,7H-pyrazolo[4,3-c]pyridin-5-yl}-4-oxoquinazolin-3-yl)piperidine-1-carboxylate (50 mg, 88%) as a solid. LCMS (ES, m/z): 483 [M+H]⁺.

### Synthesis of Compound 365

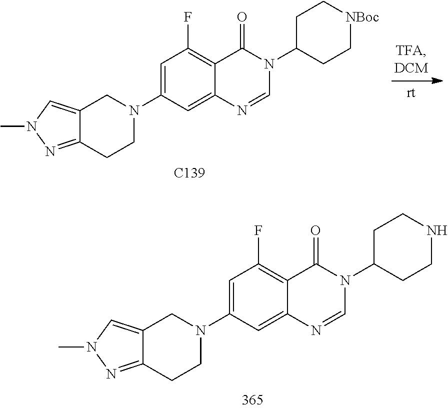 [SMILES: Cn1cc2c(n1)CCN(c1cc(F)c3c(=O)n(C4CCN(C(=O)OC(C)(C)C)CC4)cnc3c1)C2 ; "C139" ; Cn1cc2c(n1)CCN(c1cc(F)c3c(=O)n(C4CCNCC4)cnc3c1)C2]

A mixture of tert-butyl 4-(5-fluoro-7-(2-methyl-2,4,6,7-tetrahydro-5H-pyrazolo[4,3-c]pyridin-5-yl)-4-oxoquinazolin-3(4H)-yl)piperidine-1-carboxylate (50 mg, 0.100 mmol, 1 equiv), DCM (1 mL) and TFA (1 mL, 13.463 mmol, 134.52 equiv) was stirred for 1 h at room temperature. The resulting mixture was concentrated under reduced pressure residue. The residue was purified by reverse phase flash (Condition 4, Gradient 1) to afford 5-fluoro-7-(2-methyl-2,4,6,7-tetrahydro-5H-pyrazolo[4,3-c]pyridin-5-yl)-3-(piperidin-4-yl)quinazolin-4(3H)-one (12 mg, 25%) as a solid. LCMS (ES, m/z): 383 [M+H]⁺. ¹H NMR (300 MHz, Chloroform-d) δ 8.02 (s, 1H), 7.22 (s, 1H), 6.82 (d, J=2.6 Hz, 1H), 6.74 (d, J=14.4 Hz, 1H), 4.47 (s, 2H), 3.91-3.76 (m, 5H), 3.31 (d, J=12.4 Hz, 2H), 2.97-2.80 (m, 3H), 1.97 (s, 3H), 1.93-1.82 (m, 1H).

### Example 154: Synthesis of Compound 347 and 348

### Synthesis of Intermediates C141 and C142

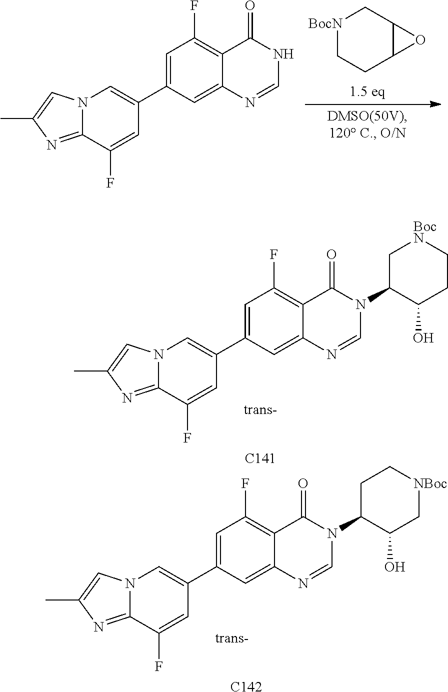 [SMILES: Cc1cn2cc(-c3cc(F)c4c(=O)[nH]cnc4c3)cc(F)c2n1 ; CC(C)(C)OC(=O)N1CCC2OC2C1 ; Cc1cn2cc(-c3cc(F)c4c(=O)n([C@H]5CN(C(=O)OC(C)(C)C)CC[C@@H]5O)cnc4c3)cc(F)c2n1 ; "C141" ; Cc1cn2cc(-c3cc(F)c4c(=O)n([C@H]5CCN(C(=O)OC(C)(C)C)C[C@@H]5O)cnc4c3)cc(F)c2n1 ; "C142"]

A solution of 5-fluoro-7-{8-fluoro-2-methylimidazo[1,2-a]pyridin-6-yl}-3H-quinazolin-4-one (1.15 g, 3.68 mmol, 1.00 equiv) and K₂CO₃ (1.53 g, 11.04 mmol, 3 equiv) in DMSO (38.33 mL) was stirred overnight at 110° C. The resulting mixture was diluted with water (30 mL) and extracted with ethyl acetate (3×30 mL). The organic layers were combined, washed with brine (1×30 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (96:4) to afford products C141 and C142 (370 mg) as solids. LCMS (ES, m/z): 512 [M+H]⁺

### Synthesis of Compounds 347 and 348

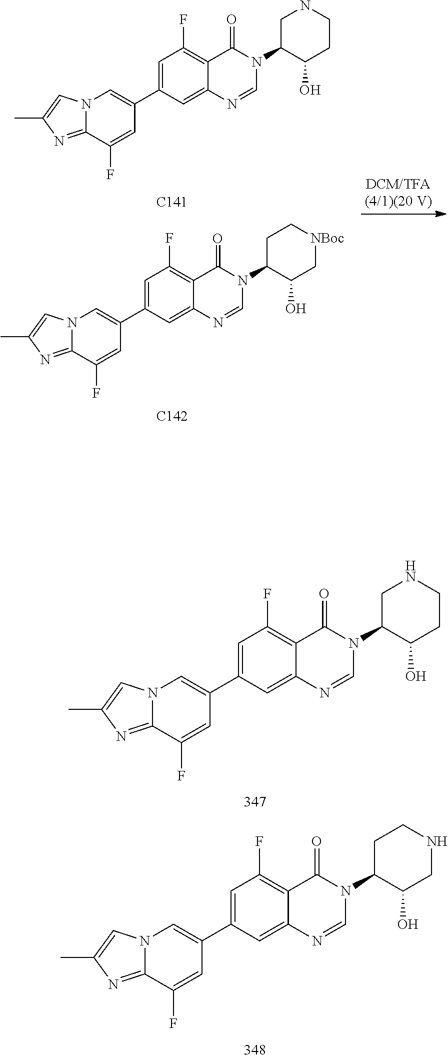

A solution of mixture products (370.0 mg, 0.72 mmol, 1.00 equiv) in trifluoroacetic acid (1 mL) and dichloromethane (4 mL) was stirred for 1 hr at room temperature. The resulting mixture was concentrated under vacuum to give a residue. The residue (400 mg) was purified by Prep-HPLC (Condition 9, Gradient 1) to afford 5-fluoro-7-{8-fluoro-2-methylimidazo[1,2-a]pyridin-6-yl}-3-[(3S,4S)-3-hydroxypiperidin-4-yl]quinazolin-4-one (8.6 mg, 3%) as a solid and 5-fluoro-7-{8-fluoro-2-methylimidazo[1,2-a]pyridin-6-yl}-3-[(3S,4S)-4-hydroxypiperidin-3-yl]quinazolin-4-one (8.7 mg, 3%) as a solid.

| Compound | |
|---|---|
| No. | Analysis Data |
| 347 | LCMS (ES, m/z): 412 [M + H]⁺ |
| | ¹H NMR (400 MHz, DMSO-d₆) δ 9.08 (d, J = 1.6 Hz, 1H), 8.43 (s, 1H), |
| | 7.89 (d, J = 1.8 Hz, 1H), 7.84 (d, J = 3.0 Hz, 1H), 7.75 (dd, J = 12.5, 1.6 |
| | Hz, 2H), 5.12 (d, J = 5.2 Hz, 1H), 4.42 (s, 1H), 3.95 (s, 1H), 3.16 (dd, J = |
| | 11.9, 4.7 Hz, 1H), 2.99 (d, J = 12.2 Hz, 1H), 2.55 (d, J = 11.2 Hz, 1H), 2.39 |
| | (s, 3H), 2.37-2.32 (m, 1H), 1.96 (s, 1H), 1.81 (dd, J = 12.7, 3.9 Hz, 1H). |
| | ¹⁹F NMR (376 MHz, DMSO) δ −73.40, −110.18, −131.86. |
| 348 | LCMS (ES, m/z)(PH-RMT-2021-0222-0A): 412 [M + H]⁺ |
| | ¹H NMR (400 MHz, DMSO-d₆) δ 9.06 (d, J = 1.6 Hz, 1H), 8.47 (s, 1H), |
| | 7.88 (d, J = 1.8 Hz, 1H), 7.82 (d, J = 3.0 Hz, 1H), 7.74 (dd, J = 12.5, 1.7 |
| | Hz, 2H), 5.31-4.99 (m, 1H), 4.44 (s, 1H), 4.06 (s, 1H), 3.08-2.87 (m, |
| | 2H), 2.84 (s, 1H), 2.57 (td, J = 12.9, 2.6 Hz, 1H), 2.39 (s, 3H), 2.01-1.83 |
| | (m, 1H), 1.45 (qd, J = 12.4, 4.0 Hz, 1H). |
| | ¹⁹F NMR (376 MHz, DMSO) δ −73.43, −110.61, −132.11. |

### Example 155: Synthesis of Compound 399 Synthesis of Compound 399

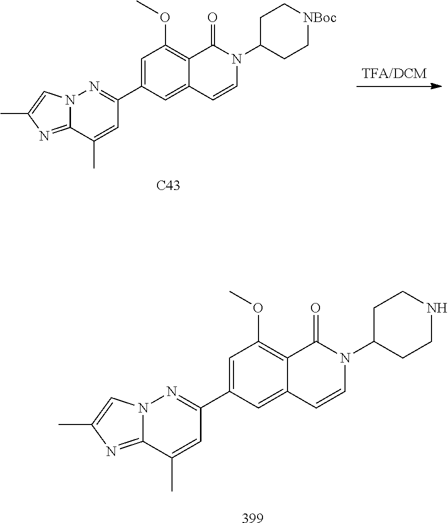 [SMILES: COc1cc(-c2cc(C)c3nc(C)cn3n2)cc2ccn(C3CCN(C(=O)OC(C)(C)C)CC3)c(=O)c12 ; "C43" ; COc1cc(-c2cc(C)c3nc(C)cn3n2)cc2ccn(C3CCNCC3)c(=O)c12 ; "399"]

A mixture of tert-butyl 4-(6-{2,8-dimethylimidazo[1,2-b]pyridazin-6-yl}-8-methoxy-1-oxoisoquinolin-2-yl)piperidine-1-carboxylate (60 mg, 0.119 mmol, 1 equiv) and trifluoroacetic acid (1 mL) in DCM (5 mL) was stirred for 1 h at 0° C., then concentrated under vacuum to give a residue. The residue was purified by Prep-HPLC (Condition 8, Gradient 3) to afford 6-{2,8-dimethylimidazo[1,2-b]pyridazin-6-yl}-8-methoxy-2-(piperidin-4-yl)isoquinolin-1-one (10 mg, 21%) as a solid. LCMS (ES, m/z): 404 [M+H]⁺. ¹H NMR (300 MHz, DMSO-d₆) δ 8.12 (d, J=1.0 Hz, 1H), 7.80 (dd, J=16.3, 1.4 Hz, 2H), 7.58-7.47 (m, 2H), 6.64 (d, J=7.5 Hz, 1H), 4.82 (dt, J=11.2, 6.1 Hz, 1H), 3.97 (s, 3H), 3.08 (d, J=12.0 Hz, 2H), 2.64 (d, J=1.0 Hz, 3H), 2.60 (s, 1H), 2.46-2.40 (m, 3H), 1.77-1.68 (m, 1H), 1.67 (s, 3H).

### Example 156: Synthesis of Compound 401

### Synthesis of Compound 401

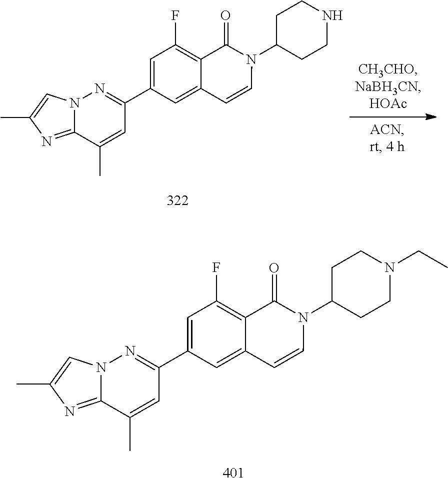 [SMILES: Cc1cn2nc(-c3cc(F)c4c(=O)n(C5CCNCC5)ccc4c3)cc(C)c2n1 ; "322" ; CCN1CCC(n2ccc3cc(-c4cc(C)c5nc(C)cn5n4)cc(F)c3c2=O)CC1 ; "401"]

To a stirred mixture of 6-{2,8-dimethylimidazo[1,2-b]pyridazin-6-yl}-8-fluoro-2-(piperidin-4-yl)isoquinolin-1-one (60 mg, 0.153 mmol, 1 equiv) and acetaldehyde (34 mg, 0.765 mmol, 5 equiv) in acetonitrile (3 mL) was added NaBH₃CN (29 mg, 0.459 mmol, 3 equiv) portionwise at room temperature, then stirred for 0.5 h. To the resulting mixture was added HOAc (92 mg, 1.530 mmol, 10 equiv) dropwise at room temperature. The reaction mixture was stirred for an additional 4 h at room temperature, then quenched with water (10 mL) and basified to pH 8 with saturated NaHCO₃ (aq.). The resulting mixture was extracted with ethyl acetate (3×5 mL). The organic layers were combined, washed with brine (1×5 mL), dried over anhydrous Na₂SO₄, and filtered. The filtrate was concentrated under reduced pressure to give a residue. The residue was purified by reverse flash chromatography (Condition 9, Gradient 1) to afford 6-{2,8-dimethylimidazo[1,2-b]pyridazin-6-yl}-2-(1-ethylpiperidin-4-yl)-8-fluoroisoquinolin-1-one hydrochloride (2.7 mg, 4%) as a solid. LCMS (ES, m/z): 420 [M+H]⁺. ¹H NMR (300 MHz, Methanol-d₄) δ 8.38-8.23 (m, 3H), 7.99 (d, J=12.8 Hz, 1H), 7.51 (d, J=7.7 Hz, 1H), 6.91 (d, J=8.1 Hz, 1H), 5.11 (s, 1H), 3.80 (d, J=12.5 Hz, 2H), 3.28 (s, 2H), 3.24 (s, 1H), 2.82 (s, 3H), 2.69 (d, J=12.8 Hz, 4H), 2.41 (d, J=13.3 Hz, 2H), 2.25 (d, J=13.8 Hz, 2H), 1.43 (t, J=7.3 Hz, 3H).

### Example 157: Synthesis of Compound 507

### Synthesis of Intermediate C144

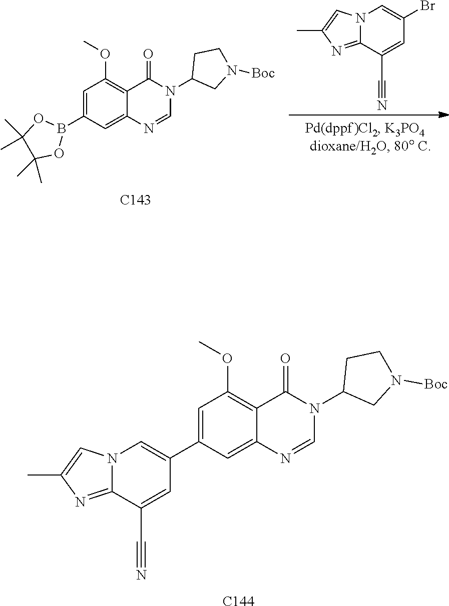 [SMILES: COc1cc(B2OC(C)(C)C(C)(C)O2)cc2ncn(C3CCN(C(=O)OC(C)(C)C)C3)c(=O)c12 ; Cc1cn2cc(Br)cc(C#N)c2n1 ; "C143" ; COc1cc(-c2cc(C#N)c3nc(C)cn3c2)cc2ncn(C3CCN(C(=O)OC(C)(C)C)C3)c(=O)c12 ; "C144"]

A mixture of C143, water (1.0 mL), 6-bromo-2-methylimidazo[1,2-a]pyridine-8-carbonitrile (262.9 mg, 1.115 mmol, 1.5 equiv), K₃PO₄ (157.61 mg, 0.743 mmol, 1.0 equiv), and Pd(dppf)Cl₂ (54.33 mg, 0.074 mmol, 0.1 equiv) was stirred overnight at 80° C. under nitrogen atmosphere. The resulting mixture was concentrated under reduced pressure to give a residue. The residue was purified by silica gel column chromatography, eluted with CH₂Cl₂/MeOH (20:1) to afford tert-butyl 3-(7-{8-cyano-2-methylimidazo[1,2-a]pyridin-6-yl}-5-methoxy-4-oxoquinazolin-3-yl)pyrrolidine-1-carboxylate (300 mg, 81%) as a solid. LCMS (ES, m/z): 501 [M+H]⁺

### Synthesis of Compound 507

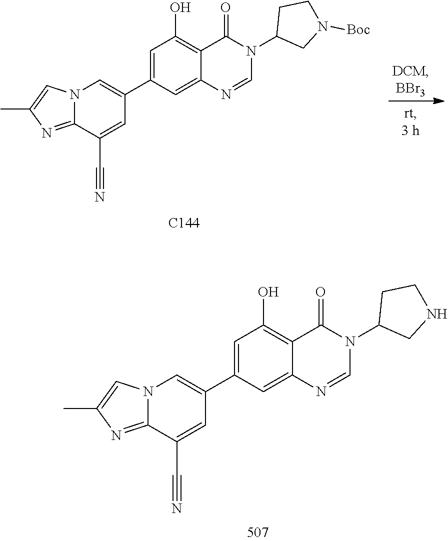 [SMILES: Cc1cn2cc(-c3cc(O)c4c(=O)n(C5CCN(C(=O)OC(C)(C)C)C5)cnc4c3)cc(C#N)c2n1 ; "C144" ; Cc1cn2cc(-c3cc(O)c4c(=O)n(C5CCNC5)cnc4c3)cc(C#N)c2n1]

A mixture of tert-butyl 3-(7-{8-cyano-2-methylimidazo[1,2-a]pyridin-6-yl}-5-methoxy-4-oxoquinazolin-3-yl)pyrrolidine-1-carboxylate (100 mg, 0.200 mmol, 1.0 equiv), DCM (1 mL), and BBr₃ (250.24 mg, 1.0 mmol, 5.0 equiv) was stirred for 3 h at room temperature. The reaction mixture was poured into a mixture of water and ice. The resulting mixture was purified by reverse phase flash (Condition 3, Gradient 1) to afford 6-[5-hydroxy-4-oxo-3-(pyrrolidin-3-yl)quinazolin-7-yl]-2-methylimidazo[1,2-a]pyridine-8-carbonitrile; trifluoroacetic acid (30 mg, 30%) as a solid. LCMS (ES, m/z): 387 [M+H]⁺. ¹H NMR (400 MHz, DMSO-d₆) δ 11.65 (s, 1H), 9.43 (d, J=1.8 Hz, 1H), 9.09 (s, 1H), 8.83 (s, 1H), 8.49 (d, J=1.8 Hz, 1H), 8.43 (s, 1H), 7.90 (d, J=1.1 Hz, 1H), 7.62 (d, J=1.7 Hz, 1H), 7.39 (d, J=1.7 Hz, 1H), 5.20 (ddd, J=14.5, 9.0, 5.7 Hz, 1H), 3.68 (dt, J=10.0, 5.8 Hz, 1H), 3.66-3.58 (m, 1H), 3.31 (dt, J=12.2, 6.6 Hz, 2H), 2.51-2.35 (m, 2H), 2.43 (s, 3H).

### Example 158: Synthesis of Compound 508

### Synthesis of Intermediate C145

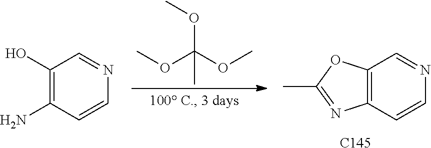

A mixture of 4-aminopyridin-3-ol (2 g, 18.163 mmol, 1 equiv) and 1,1,1-trimethoxyethane (60 mL) was stirred for 3 days at 100° C. The resulting mixture was filtered, and the filtrate was concentrated under reduced pressure to give a residue. The residue was purified by silica gel column chromatography, eluted with PE/EA (5:1) to afford 2-methyl-[1,3]oxazolo[5,4-c]pyridine (1.5 g, 62%) as an oil. LCMS (ES, m/z): 135 [M+H]⁺.

### Synthesis of Intermediate C146

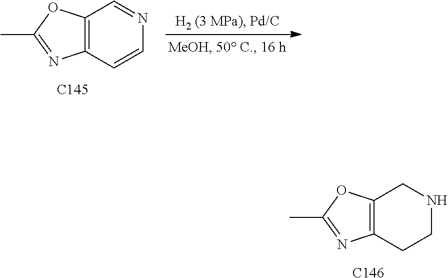

To a solution of 2-methyl-[1,3]oxazolo[5,4-c]pyridine (300 mg, 2.237 mmol, 1 equiv) in methanol (50 mL) was added Pd/C (10%, 100 mg). The resulting mixture was hydrogenated at room temperature under 30 psi of hydrogen pressure overnight, then filtered and concentrated under reduced pressure to give a residue. The residue was purified by silica gel column chromatography, eluted with PE/EA (5:1) to afford 2-methyl-4H,5H,6H,7H-[1,3]oxazolo[5,4-c]pyridine (96 mg, 31%) as an oil. LCMS (ES, m/z): 139 [M+H]⁺.

### Synthesis of Intermediate C147

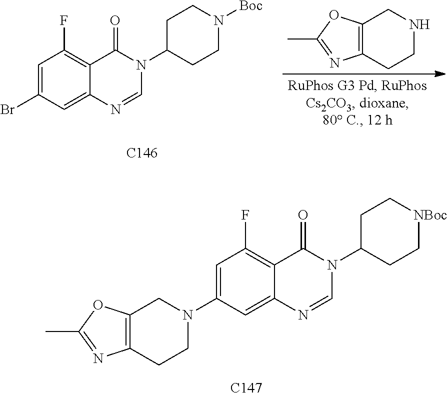

To a stirred mixture of tert-butyl 4-(7-bromo-5-fluoro-4-oxoquinazolin-3-yl)piperidine-1-carboxylate (100 mg, 0.235 mmol, 1 equiv) and 2-methyl-4H,5H,6H,7H-[1,3]oxazolo[5,4-c]pyridine (38.90 mg, 0.282 mmol, 1.2 equiv) in dioxane (2 mL) was added Cs₂CO₃ (229.30 mg, 0.705 mmol, 3 equiv), RuPhos (21.89 mg, 0.047 mmol, 0.2 equiv), and RuPhos Palladacycle Gen.3 (19.62 mg, 0.024 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (20:1) to afford tert-butyl 4-(5-fluoro-7-{2-methyl-4H,6H,7H-[1,3]oxazolo[5,4-c]pyridin-5-yl}-4-oxoquinazolin-3-yl)piperidine-1-carboxylate (80 mg, 71%) as a solid. LCMS (ES, m/z): 484 [M+H]⁺.

### Synthesis of Compound 508

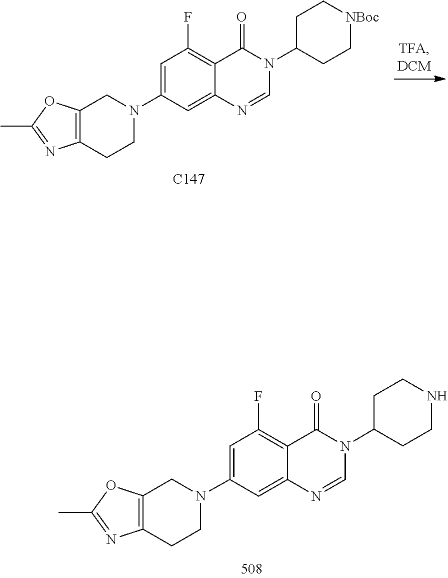 [SMILES: Cc1nc2c(o1)CN(c1cc(F)c3c(=O)n(C4CCN(C(=O)OC(C)(C)C)CC4)cnc3c1)CC2 ; "C147" ; Cc1nc2c(o1)CN(c1cc(F)c3c(=O)n(C4CCNCC4)cnc3c1)CC2]

To a stirred solution of tert-butyl 4-(5-fluoro-7-{2-methyl-4H,6H,7H-[1,3]oxazolo[5,4-c]pyridin-5-yl}-4-oxoquinazolin-3-yl)piperidine-1-carboxylate (80 mg, 0.165 mmol, 1 equiv) in DCM (1 mL) was added TFA (1 mL) at room temperature. The resulting mixture was stirred for 2 h at room temperature, then concentrated under reduced pressure to give a residue. The residue was purified by Prep-HPLC (Condition 6, Gradient 3) to afford 5-fluoro-7-{2-methyl-4H,6H,7H-[1,3]oxazolo[5,4-c]pyridin-5-yl}-3-(piperidin-4-yl)quinazolin-4-one; trifluoroacetic acid (14.8 mg, 18%) as a solid. LCMS (ES, m/z): 384 [M+H]. ¹H NMR (400 MHz, Methanol-d₄) δ 8.16 (s, 1H), 7.03 (dd, J=14.8, 2.5 Hz, 1H), 6.90 (d, J=2.5 Hz, 1H), 4.81-4.72 (m, 1H), 4.54 (d, J=2.2 Hz, 2H), 3.87 (t, J=5.6 Hz, 2H), 3.65-3.57 (m, 2H), 3.30-3.18 (m, 2H), 2.77-2.69 (m, 2H), 2.47 (s, 5H), 2.20 (d, J=13.6 Hz, 2H).

### Example 159: Synthesis of Compound 366

### Synthesis of Intermediate C148

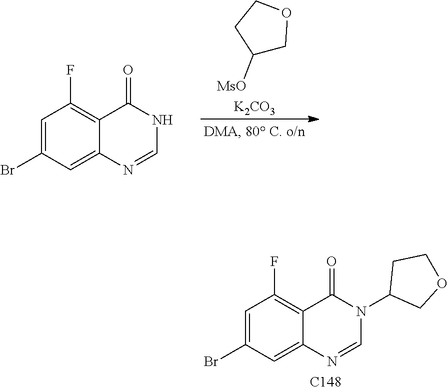

A mixture of 7-bromo-5-fluoro-3H-quinazolin-4-one (500 mg, 2.057 mmol, 1.0 equiv), DMA (5 mL), oxolan-3-yl methanesulfonate (410.29 mg, 2.468 mmol, 1.2 equiv), and K₂CO₃ (1137.33 mg, 8.228 mmol, 4.0 equiv) was stirred overnight at 80° C. The resulting mixture was filtered, and the filter cake washed with DCM (3×20 mL). The filtrate was concentrated under reduced pressure to give a residue. The residue was purified by reverse flash chromatography (Condition 3, Gradient 1) to afford 7-bromo-5-fluoro-3-(oxolan-3-yl)quinazolin-4-one (208 mg, 33%) as a solid. LCMS (ES, m/z): 313 [M+H]⁺.

### Synthesis of Intermediate C149

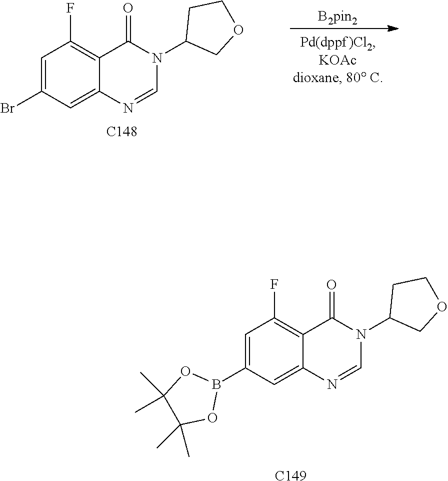 [SMILES: O=c1c2c(F)cc(Br)cc2ncn1C1CCOC1 ; "C148" ; CC1(C)OB(c2cc(F)c3c(=O)n(C4CCOC4)cnc3c2)OC1(C)C ; "C149"]

A mixture of 7-bromo-5-fluoro-3-(oxolan-3-yl)quinazolin-4-one (100 mg, 0.319 mmol, 1.0 equiv), 4,4,5,5-tetramethyl-2-(tetramethyl-1,3,2-dioxaborolan-2-yl)-1,3,2-dioxaborolane (97.32 mg, 0.383 mmol, 1.2 equiv), AcOK (94.03 mg, 0.957 mmol, 3.0 equiv), dioxane (1 mL) and Pd(dppf)Cl₂ (23.37 mg, 0.032 mmol, 0.1 equiv) was stirred for 4 h at 80° C. under nitrogen atmosphere.

### Synthesis of Intermediate C150

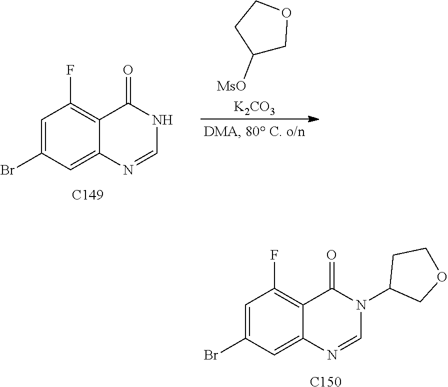

A mixture of 7-bromo-5-fluoro-3H-quinazolin-4-one (500 mg, 2.057 mmol, 1.0 equiv), DMA (5 mL), oxolan-3-yl methanesulfonate (410.29 mg, 2.468 mmol, 1.2 equiv), and K₂CO₃ (1137.33 mg, 8.228 mmol, 4.0 equiv) was stirred overnight at 80° C. The resulting mixture was filtered, and the filter cake was washed with DCM (3×20 mL). The filtrate was concentrated under reduced pressure to give a residue. The residue was purified by reverse flash chromatography (Condition 3, Gradient 1) to afford 7-bromo-5-fluoro-3-(oxolan-3-yl)quinazolin-4-one (208 mg, 33%) as a solid. LCMS (ES, m/z): 313 [M+H]⁺.

### Synthesis of Intermediate C151

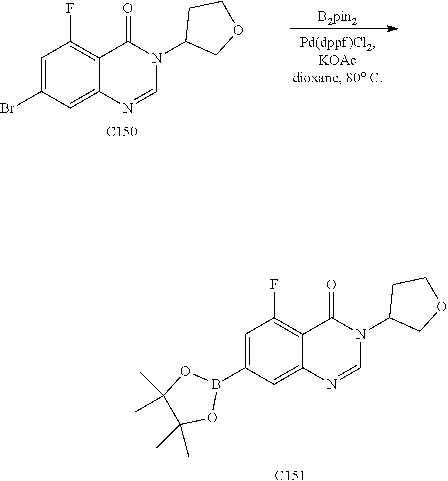 [SMILES: O=c1c2c(F)cc(Br)cc2ncn1C1CCOC1 ; "C150" ; CC1(C)OB(c2cc(F)c3c(=O)n(C4CCOC4)cnc3c2)OC1(C)C ; "C151"]

A mixture of 7-bromo-5-fluoro-3-(oxolan-3-yl)quinazolin-4-one (100 mg, 0.319 mmol, 1.0 equiv), 4,4,5,5-tetramethyl-2-(tetramethyl-1,3,2-dioxaborolan-2-yl)-1,3,2-dioxaborolane (97.32 mg, 0.383 mmol, 1.2 equiv), AcOK (94.03 mg, 0.957 mmol, 3.0 equiv), dioxane (1 mL) and Pd(dppf)Cl₂ (23.37 mg, 0.032 mmol, 0.1 equiv) was stirred for 4 h at 80° C. under nitrogen atmosphere. LCMS (ES, m/z): 361 [M+H]⁺.

### Synthesis of Compound 366

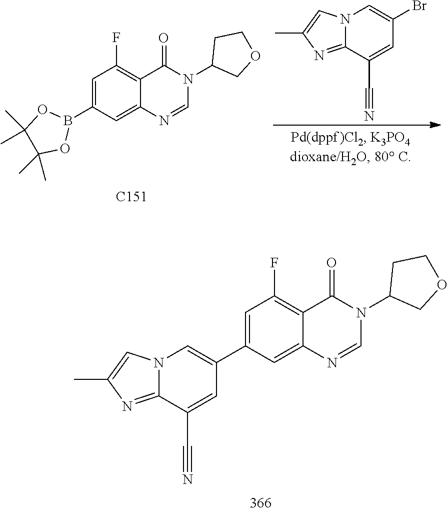

To a stirred mixture of C151 was added water (0.2 mL), 6-bromo-2-methylimidazo[1,2-a]pyridine-8-carbonitrile (150.74 mg, 0.638 mmol, 2.0 equiv), K₃PO₄ (135.54 mg, 0.638 mmol, 2.0 equiv), and Pd(dppf)Cl₂ (23.36 mg, 0.032 mmol, 0.1 equiv) at room temperature under nitrogen atmosphere. The resulting mixture was stirred overnight at 80° C. under nitrogen atmosphere. The resulting mixture was concentrated under reduced pressure to give a residue. The residue was purified by reverse phase flash (Condition 5, Gradient 4) to afford 6-[5-fluoro-4-oxo-3-(oxolan-3-yl)quinazolin-7-yl]-2-methylimidazo[1,2-a]pyridine-8-carbonitrile (35 mg, 28%) as a solid. LCMS (ES, m/z): 390 [M+H]⁺. ¹H NMR (300 MHz, DMSO-d₆) δ 9.48 (d, J=1.8 Hz, 1H), 8.54 (d, J=1.8 Hz, 1H), 8.30 (s, 1H), 7.97 (d, J=1.8 Hz, 1H), 7.90 (d, J=1.1 Hz, 1H), 7.82 (dd, J=12.6, 1.8 Hz, 1H), 5.33 (td, J=6.0, 3.1 Hz, 1H), 4.20-4.07 (m, 1H), 4.01 (dd, J=10.1, 2.9 Hz, 1H), 3.94-3.73 (m, 2H), 2.44 (d, J=0.9 Hz, 3H), 2.24-2.14 (m, 2H). (ES, m/z): 361 [M+H]⁺.

### Example 160: Synthesis of Compound 504

### Synthesis of Compound 504

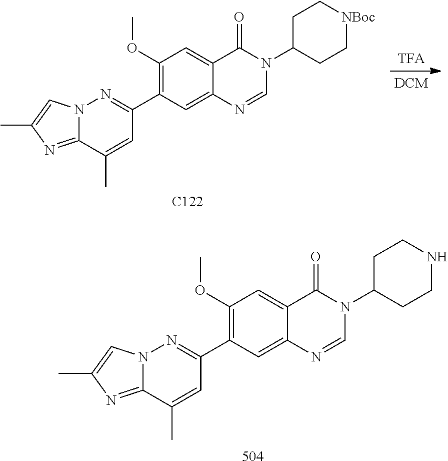 [SMILES: COc1cc2c(=O)n(C3CCN(C(=O)OC(C)(C)C)CC3)cnc2cc1-c1cc(C)c2nc(C)cn2n1 ; "C122" ; COc1cc2c(=O)n(C3CCNCC3)cnc2cc1-c1cc(C)c2nc(C)cn2n1 ; "504" ; "TFA"]

A mixture of tert-butyl 4-(7-{2,8-dimethylimidazo[1,2-b]pyridazin-6-yl}-6-methoxy-4-oxoquinazolin-3-yl)piperidine-1-carboxylate (50 mg, 0.099 mmol, 1 equiv), DCM (1 mL), and TFA (1 mL) was stirred for 3 h at room temperature. The resulting mixture was concentrated under reduced pressure to give a residue. The residue was purified by reverse phase flash chromatography (Condition 4, Gradient 1) to afford 7-{2,8-dimethylimidazo[1,2-b]pyridazin-6-yl}-6-methoxy-3-(piperidin-4-yl)quinazolin-4-one (11 mg, 27%) as a solid. LCMS (ES, m/z): 405 [M+H]⁺. ¹H NMR (400 MHz, DMSO-d₆) δ 8.38 (s, 1H), 8.08 (s, 1H), 7.82 (s, 1H), 7.71 (s, 1H), 7.32 (d, J=1.3 Hz, 1H), 4.72 (t, J=12.3 Hz, 1H), 3.94 (s, 3H), 3.13 (d, J=12.2 Hz, 2H), 2.73-2.62 (m, 2H), 2.58 (d, J=1.1 Hz, 3H), 2.42 (s, 3H), 1.97 (q, J=12.0, 11.6 Hz, 2H), 1.81 (d, J=11.9 Hz, 2H).

### Example 161: Synthesis of Compound 505

### Synthesis of Intermediate C152

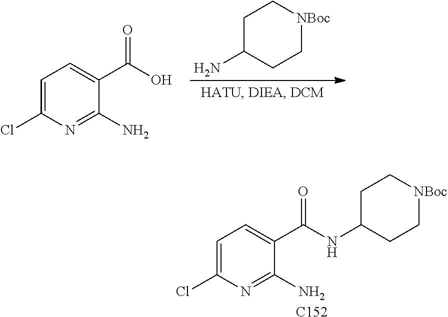 [SMILES: Nc1nc(Cl)ccc1C(=O)O ; CC(C)(C)OC(=O)N1CCC(N)CC1 ; CC(C)(C)OC(=O)N1CCC(NC(=O)c2ccc(Cl)nc2N)CC1 ; "C152"]

To a stirred solution of 2-amino-6-chloropyridine-3-carboxylic acid (2 g, 11.589 mmol, 1 equiv) and tert-butyl 4-aminopiperidine-1-carboxylate (2.8 g, 13.907 mmol, 1.2 equiv) in DCM (30 mL) was added HATU (5.3 g, 13.907 mmol, 1.2 equiv) and DIEA (4.5 g, 34.767 mmol, 3 equiv) portionwise at room temperature. The resulting mixture was stirred for 5 h, then quenched with water (100 mL) at room temperature and extracted with CH₂Cl₂ (3×20 mL). The organic layers were combined, washed with brine (1×50 mL), dried over anhydrous Na₂SO₄, and filtered. The filtrate was concentrated under reduced pressure to give a residue. The residue was purified by silica gel column chromatography, eluted with PE/EA (2:1) to afford tert-butyl 4-(2-amino-6-chloropyridine-3-amido)piperidine-1-carboxylate (3.8 g, 92%) as a solid. LCMS (ES, m/z): 355 [M+H]⁺.

### Synthesis of Intermediate C153

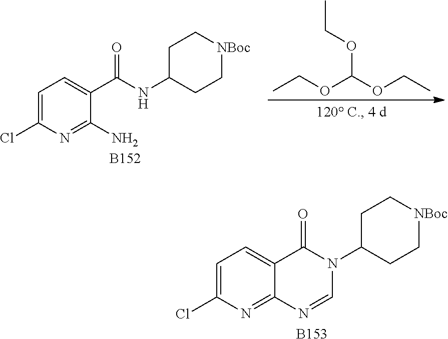

A solution of tert-butyl 4-(2-amino-6-chloropyridine-3-amido)piperidine-1-carboxylate (2 g, 5.636 mmol, 1 equiv) in CH(OCH₃)₃(10 mL) was stirred for 4 days at 120° C., then cooled to room temperature. The resulting mixture was concentrated under reduced pressure to give a residue. The residue was purified by silica gel column chromatography, eluted with PE/EA (2:1) to afford tert-butyl 4-{7-chloro-4-oxopyrido[2,3-d]pyrimidin-3-yl}piperidine-1-carboxylate (310 mg, 15%) as a solid. LCMS (ES, m/z): 365 [M+H]⁺.

### Synthesis of Intermediate C154

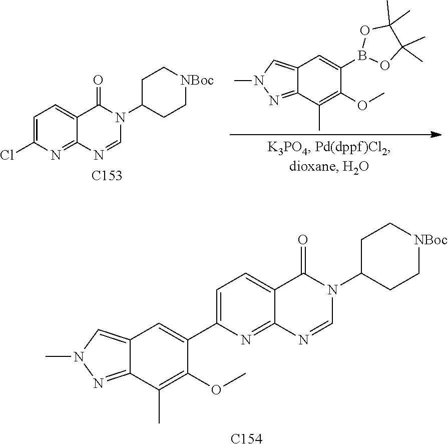

To a stirred solution of tert-butyl 4-{7-chloro-4-oxopyrido[2,3-d]pyrimidin-3-yl}piperidine-1-carboxylate (300 mg, 0.822 mmol, 1 equiv) and 6-methoxy-2,7-dimethyl-5-(4,4,5,5-tetramethyl-1,3,2-dioxaborolan-2-yl)indazole (209 mg, 0.690 mmol, 0.84 equiv) in dioxane (5 mL) and water (1 mL) was added K₃PO₄ (524 mg, 2.466 mmol, 3 equiv) and Pd(dppf)Cl₂ (60 mg, 0.082 mmol, 0.1 equiv) portionwise at room temperature under nitrogen atmosphere. The resulting mixture was stirred for 3 h at 80° C. under nitrogen atmosphere, then cooled to room temperature. The resulting mixture was concentrated under vacuum to give a residue. The residue was purified by silica gel column chromatography, eluted with PE/EA (1:10) to afford tert-butyl 4-[7-(6-methoxy-2,7-dimethylindazol-5-yl)-4-oxopyrido[2,3-d]pyrimidin-3-yl]piperidine-1-carboxylate (400 mg, 96%) as a solid. LCMS (ES, m/z): 505 [M+H]⁺.

### Synthesis of Compound 505

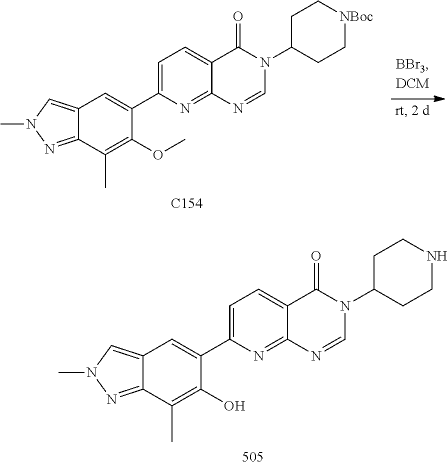 [SMILES: COc1c(-c2ccc3c(=O)n(C4CCN(C(=O)OC(C)(C)C)CC4)cnc3n2)cc2cn(C)nc2c1C ; "C154" ; Cc1c(O)c(-c2ccc3c(=O)n(C4CCNCC4)cnc3n2)cc2cn(C)nc12 ; "505"]

To a stirred solution of tert-butyl 4-[7-(6-methoxy-2,7-dimethylindazol-5-yl)-4-oxopyrido[2,3-d]pyrimidin-3-yl]piperidine-1-carboxylate (300 mg, 0.595 mmol, 1 equiv) in DCM (8 mL) was added BBr₃ (1 mL) dropwise at room temperature. The resulting mixture was stirred for 2 days, then quenched with water (2 mL) at room temperature. The resulting mixture was concentrated under reduced pressure to a residue. The residue was purified by reverse flash chromatography (Condition 5, Gradient 1) to afford 7-(6-hydroxy-2,7-dimethyl-2H-indazol-5-yl)-3-(piperidin-4-yl)pyrido[2,3-d]pyrimidin-4(3H)-one (30 mg, 13%) as a solid. LCMS (ES, m/z): 391 [M+H]⁺. ¹H NMR (300 MHz, DMSO-d₆) δ 8.62 (d, J=8.6 Hz, 1H), 8.56 (d, J=2.2 Hz, 2H), 8.39 (d, J=9.1 Hz, 2H), 4.88-4.74 (m, 1H), 4.14 (s, 3H), 3.48 (d, J=13.0 Hz, 2H), 3.20-3.06 (m, 2H), 2.36 (s, 3H), 2.30 (m, J=12.7, 4.0 Hz, 2H), 2.18-2.06 (m, 2H).

### Example 162: Synthesis of Compound 506

### Synthesis of Compound 506

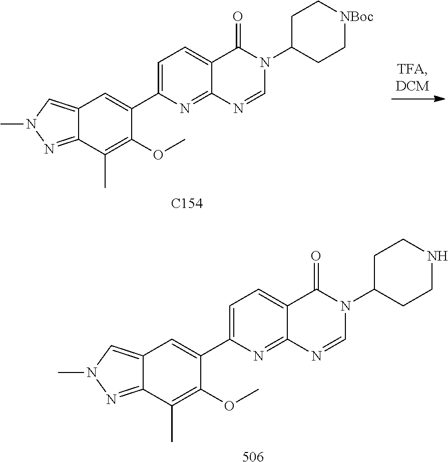 [SMILES: COc1c(-c2ccc3c(=O)n(C4CCN(C(=O)OC(C)(C)C)CC4)cnc3n2)cc2cn(C)nc2c1C ; "C154" ; COc1c(-c2ccc3c(=O)n(C4CCNCC4)cnc3n2)cc2cn(C)nc2c1C ; "506"]

A mixture of tert-butyl 4-[7-(6-methoxy-2,7-dimethylindazol-5-yl)-4-oxopyrido[2,3-d]pyrimidin-3-yl]piperidine-1-carboxylate (80 mg, 0.159 mmol, 1 equiv) in trifluoroacetic acid (2 mL) and DCM (2 mL) was stirred for 2 h at room temperature, then concentrated under vacuum to give a residue. The residue was basified to pH 8 with 7 M NH₃(g) in methanol, then concentrated under vacuum to give a residue. The residue was purified by reverse flash chromatography (Condition 2, Gradient 9) to afford 7-(6-methoxy-2,7-dimethylindazol-5-yl)-3-(piperidin-4-yl)pyrido[2,3-d]pyrimidin-4-one (20 mg, 31%) as a solid. LCMS (ES, m/z): 405 [M+H]⁺. ¹H NMR (300 MHz, DMSO-d₆) δ 8.67 (s, 1H), 8.56 (d, J=8.3 Hz, 1H), 8.45 (s, 1H), 8.02-7.92 (m, 2H), 4.67 (t, J=12.2 Hz, 1H), 4.19 (s, 3H), 3.46 (s, 3H), 3.11 (d, J=12.2 Hz, 2H), 2.73-2.55 (m, 2H), 2.50 (s, 3H), 2.08-1.86 (m, 2H), 1.81 (d, J=11.5 Hz, 2H).

### Example 163: Synthesis of Compound 585

### Synthesis of Intermediate C155

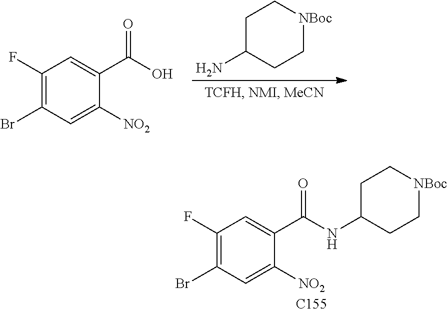 [SMILES: O=C(O)c1cc(F)c(Br)cc1[N+](=O)[O-] ; CC(C)(C)OC(=O)N1CCC(N)CC1 ; CC(C)(C)OC(=O)N1CCC(NC(=O)c2cc(F)c(Br)cc2[N+](=O)[O-])CC1 ; "C155"]

To a stirred mixture of 4-bromo-5-fluoro-2-nitrobenzoic acid (2 g, 7.576 mmol, 1 equiv) and tert-butyl 4-aminopiperidine-1-carboxylate (1.67 g, 8.334 mmol, 1.1 equiv) in CH₃CN (20 mL) was added NMI (1.87 g, 22.728 mmol, 3 equiv) and TCFH (2.76 g, 9.849 mmol, 1.3 equiv) at room temperature. The resulting mixture was stirred for 2 h, then quenched with water (10 mL) at room temperature and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by silica gel column chromatography, eluted with PE/EA (1:1) to afford tert-butyl 4-(4-bromo-5-fluoro-2-nitrobenzamido)piperidine-1-carboxylate (2.38 g, 67%) as a solid. LCMS (ES, m/z): 446 [M+H]⁺.

### Synthesis of Intermediate C156

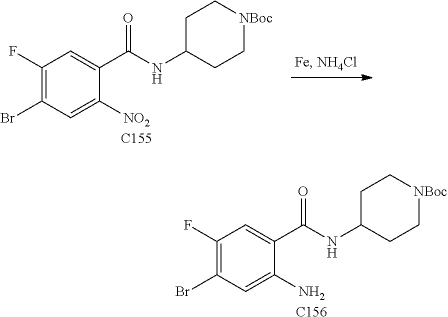 [SMILES: CC(C)(C)OC(=O)N1CCC(NC(=O)c2cc(F)c(Br)cc2[N+](=O)[O-])CC1 ; "C155" ; CC(C)(C)OC(=O)N1CCC(NC(=O)c2cc(F)c(Br)cc2N)CC1 ; "C156"]

To a stirred mixture of tert-butyl 4-(4-bromo-5-fluoro-2-nitrobenzamido)piperidine-1-carboxylate (10 g, 22.408 mmol, 1 equiv) and NH₄Cl (11.99 g, 224.080 mmol, 10 equiv) in methanol (90 mL) and water (30 mL) was added Fe (6.26 g, 112.040 mmol, 5 equiv) in portions at 25° C. The resulting mixture was stirred for 1 h at 80° C., then filtered and the filter cake washed with methanol (3×100 mL). The filtrate was concentrated under reduced pressure to give a residue. The residue was purified by silica gel column chromatography, eluted with PE/EA (2:1) to afford tert-butyl 4-(2-amino-4-bromo-5-fluorobenzamido)piperidine-1-carboxylate (5 g, 54%) as a solid. LCMS (ES, m/z): 416 [M+H]⁺.

### Synthesis of Intermediate C157

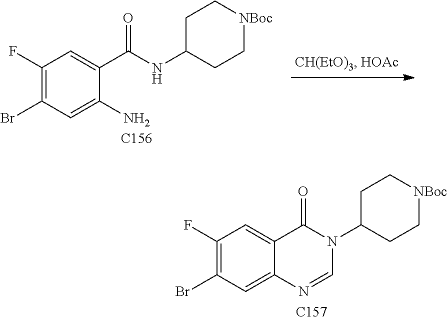 [SMILES: CC(C)(C)OC(=O)N1CCC(NC(=O)c2cc(F)c(Br)cc2N)CC1 ; "C156" ; CC(C)(C)OC(=O)N1CCC(n2cnc3cc(Br)c(F)cc3c2=O)CC1 ; "C157"]

A solution of tert-butyl 4-(2-amino-4-bromo-5-fluorobenzamido)piperidine-1-carboxylate (2 g, 4.804 mmol, 1 equiv) in CH₃COOH (10 mL) and (diethoxymethoxy)ethane (10 mL) was stirred for 3 h at 110° C., then cooled to room temperature. The resulting mixture was quenched with water (50 mL) at room temperature. A precipitate formed that was collected by filtration and washed with ethyl ether (3×30 mL) to afford tert-butyl 4-(7-bromo-6-fluoro-4-oxoquinazolin-3-yl)piperidine-1-carboxylate (1.3 g, 63%) as a solid. LCMS (ES, m/z): 426 [M+H]⁺.

### Synthesis of Intermediate C158

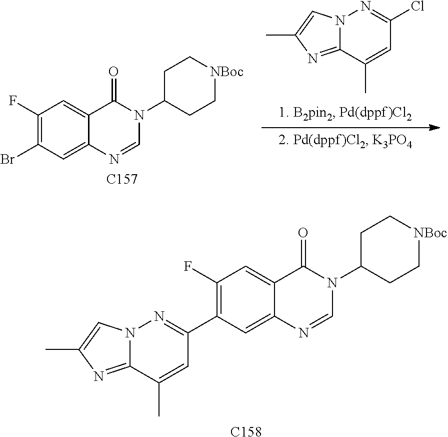 [SMILES: CC(C)(C)OC(=O)N1CCC(n2cnc3cc(Br)c(F)cc3c2=O)CC1 ; Cc1cn2nc(Cl)cc(C)c2n1 ; "C157" ; Cc1cn2nc(-c3cc4ncn(C5CCN(C(=O)OC(C)(C)C)CC5)c(=O)c4cc3F)cc(C)c2n1 ; "C158"]

To a mixture of 6-chloro-2,8-dimethylimidazo[1,2-b]pyridazine (200 mg, 1.101 mmol, 2.35 equiv) and 4,4,5,5-tetramethyl-2-(tetramethyl-1,3,2-dioxaborolan-2-yl)-1,3,2-dioxaborolane (178.7 mg, 0.704 mmol, 1.5 equiv) in dioxane (5 mL) was added KOAc (138.1 mg, 1.407 mmol, 3.0 equiv) and Pd(dppf)Cl₂CH₂Cl₂ (38.2 mg, 0.047 mmol, 0.1 equiv). The resulting mixture was stirred for 12 h at 100° C. under a nitrogen atmosphere, then cooled to room temperature. To the reaction mixture was added tert-butyl 4-(7-bromo-6-fluoro-4-oxoquinazolin-3-yl)piperidine-1-carboxylate (200 mg, 0.469 mmol, 1 equiv), dioxane (5 mL), H₂O (1 mL), K₃PO₄ (298.7 mg, 1.407 mmol, 3.0 equiv), and Pd(dppf)Cl₂CH₂Cl₂ (38.2 mg, 0.047 mmol, 0.1 equiv) under nitrogen atmosphere. The resulting mixture was stirred for an additional 1 h at 80° C., then diluted with water (10 mL) and extracted with ethyl acetate (3×10 mL). The organic layers were combined, washed with brine (1×5 mL), dried over anhydrous Na₂SO₄, and filtered. The filtrate was concentrated under reduced pressure to give a residue. The residue was purified by reverse flash chromatography (Condition 2, Gradient 1) to afford tert-butyl 4-(7-{2,8-dimethylimidazo[1,2-b]pyridazin-6-yl}-6-fluoro-4-oxoquinazolin-3-yl)piperidine-1-carboxylate (180 mg, 78%) as a solid. LCMS (ES, m/z): 493 [M+H]⁺.

### Synthesis of Compound 585

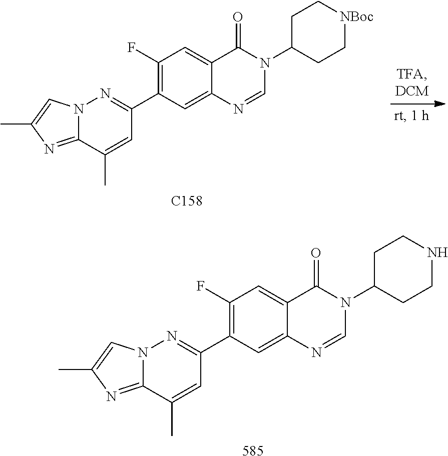 [SMILES: Cc1cn2nc(-c3cc4ncn(C5CCN(C(=O)OC(C)(C)C)CC5)c(=O)c4cc3F)cc(C)c2n1 ; "C158" ; Cc1cn2nc(-c3cc4ncn(C5CCNCC5)c(=O)c4cc3F)cc(C)c2n1 ; "585"]

A mixture of tert-butyl 4-(7-{2,8-dimethylimidazo[1,2-b]pyridazin-6-yl}-6-fluoro-4-oxoquinazolin-3-yl)piperidine-1-carboxylate (60 mg, 0.122 mmol, 1 equiv) and TFA (0.2 mL) in DCM (2 mL) was stirred for 1 h at room temperature, then basified to pH 8 with NH₃(g) in methanol. The resulting mixture was concentrated under reduced pressure to give a residue. The residue was purified by reverse flash chromatography (Condition 2, Gradient 14) to afford 7-{2,8-dimethylimidazo[1,2-b]pyridazin-6-yl}-6-fluoro-3-(piperidin-4-yl)quinazolin-4-one (20 mg, 42%) as a solid. LCMS (ES, m/z): 393 [M+H]⁺. ¹H NMR (300 MHz, DMSO-d₆) δ 8.51 (s, 1H), 8.15 (d, J=1.0 Hz, 1H), 8.07 (d, J=6.8 Hz, 1H), 8.00 (d, J=10.5 Hz, 1H), 7.45 (dd, J=2.3, 1.2 Hz, 1H), 4.69 (s, 1H), 3.10 (d, J=12.0 Hz, 2H), 2.62 (d, J=1.1 Hz, 5H), 2.47-2.40 (m, 3H), 1.94 (td, J=12.4, 11.9, 8.4 Hz, 2H), 1.79 (d, J=11.5 Hz, 2H).

### Example 164: Synthesis of Compound 586

### Synthesis of Compound C159

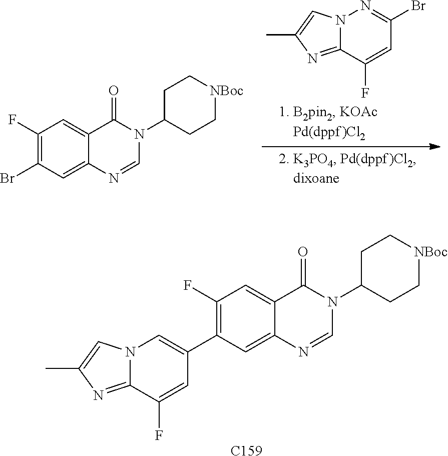 [SMILES: CC(C)(C)OC(=O)N1CCC(n2cnc3cc(Br)c(F)cc3c2=O)CC1 ; Cc1cn2nc(Br)cc(F)c2n1 ; Cc1cn2cc(-c3cc4ncn(C5CCN(C(=O)OC(C)(C)C)CC5)c(=O)c4cc3F)cc(F)c2n1 ; "C159"]

To a mixture of 6-bromo-8-fluoro-2-methylimidazo[1,2-a]pyridine (161.2 mg, 0.704 mmol, 1.5 equiv) and 4,4,5,5-tetramethyl-2-(tetramethyl-1,3,2-dioxaborolan-2-yl)-1,3,2-dioxaborolane (178.7 mg, 0.704 mmol, 1.5 equiv) in dioxane (5 mL) was added KOAc (138.1 mg, 1.407 mmol, 3.0 equiv) and Pd(dppf)Cl₂·CH₂Cl₂ (38.2 mg, 0.047 mmol, 0.1 equiv). The reaction mixture was for 12 h at 80° C. under a nitrogen atmosphere, then cooled to room temperature. To the resulting mixture was added tert-butyl 4-(7-bromo-6-fluoro-4-oxoquinazolin-3-yl)piperidine-1-carboxylate (200 mg, 0.469 mmol, 1 equiv), water (1 mL), K₃PO₄ (298.7 mg, 1.407 mmol, 3.0 equiv), and Pd(dppf)Cl₂·CH₂Cl₂ (38.2 mg, 0.047 mmol, 0.1 equiv). The reaction mixture was stirred for an additional 1 h at 80° C. under a nitrogen atmosphere, then concentrated under reduced pressure to give a residue. The residue was purified by reverse flash chromatography (Condition 2, Gradient 1) to afford tert-butyl 4-(6-fluoro-7-{8-fluoro-2-methylimidazo[1,2-a]pyridin-6-yl}-4-oxoquinazolin-3-yl)piperidine-1-carboxylate (180 mg, 77%) as a solid. LCMS (ES, m/z): 496 [M+H]⁺.

### Synthesis of Compound 586

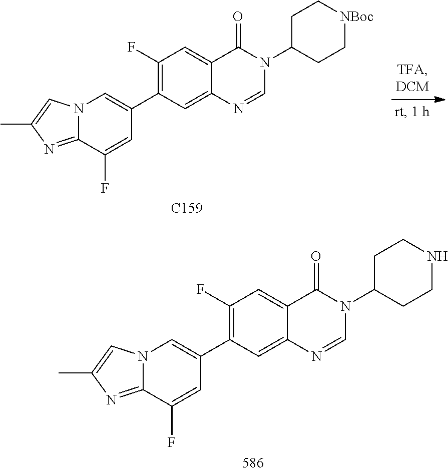 [SMILES: Cc1cn2cc(-c3cc4ncn(C5CCN(C(=O)OC(C)(C)C)CC5)c(=O)c4cc3F)cc(F)c2n1 ; "C159" ; Cc1cn2cc(-c3cc4ncn(C5CCNCC5)c(=O)c4cc3F)cc(F)c2n1 ; "586"]

A mixture of tert-butyl 4-(6-fluoro-7-{8-fluoro-2-methylimidazo[1,2-a]pyridin-6-yl}-4-oxoquinazolin-3-yl)piperidine-1-carboxylate (80 mg, 0.161 mmol, 1 equiv) and TFA (0.2 mL) in DCM (2 mL) was stirred for 1 h at room temperature, then basified to pH 8 with NH₃(g) in methanol. The resulting mixture was concentrated under reduced pressure to give a residue. The residue was purified by reverse flash chromatography (Condition 2, Gradient 3) to afford 6-fluoro-7-{8-fluoro-2-methylimidazo[1,2-a]pyridin-6-yl}-3-(piperidin-4-yl)quinazolin-4-one (37 mg, 58%) as a solid. LCMS (ES, m/z): 396 [M+H]⁺. ¹H NMR (300 MHz, DMSO-d₆) δ 8.81 (t, J=1.3 Hz, 1H), 8.49 (s, 1H), 8.02-7.88 (m, 3H), 7.48 (dt, J=12.2, 1.6 Hz, 1H), 4.75-4.61 (m, 1H), 3.10 (d, J=12.3 Hz, 2H), 2.61 (t, J=11.8 Hz, 2H), 2.40 (d, J=0.9 Hz, 3H), 2.33-2.18 (m, 1H), 2.01-1.85 (m, 2H), 1.78 (d, J=11.5 Hz, 2H).

### Example 165: Synthesis of Compound 121

### Synthesis of Intermediate C160

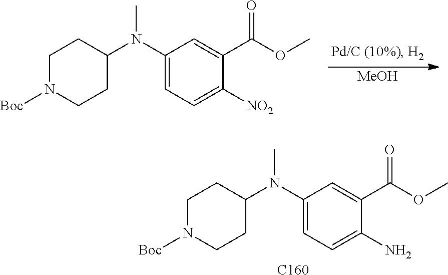 [SMILES: "MeOH" ; COC(=O)c1cc(N(C)C2CCN(C(=O)OC(C)(C)C)CC2)ccc1[N+](=O)[O-] ; COC(=O)c1cc(N(C)C2CCN(C(=O)OC(C)(C)C)CC2)ccc1N ; "C160"]

Argon was bubbled through a suspension of tert-butyl 4-((3-(methoxycarbonyl)-4-nitrophenyl)(methyl)amino)piperidine-1-carboxylate (259 mg, 0.658 mmol) in methanol (5.0 mL) for 10 min. To the solution was added Pd/C (10%) (70.0 mg, 0.066 mmol). H₂ was bubbled through the resulting mixture for 10 min and the reaction mixture stirred for 24 h under a hydrogen atmosphere. The reaction mixture was filtered through a Celite pad under reduced pressure and the filtrate concentrated in vacuo to give a residue. The residue was purified by flash chromatography using a gradient of 30-100% ethyl acetate in hexanes to afford tert-butyl 4-((4-amino-3-(methoxycarbonyl)phenyl)(methyl)amino) piperidine-1-carboxylate (209 mg, 87%) as an oil. LCMS (ES, m/z): 364.2 [M+H]⁺.

### Synthesis of Intermediate C161

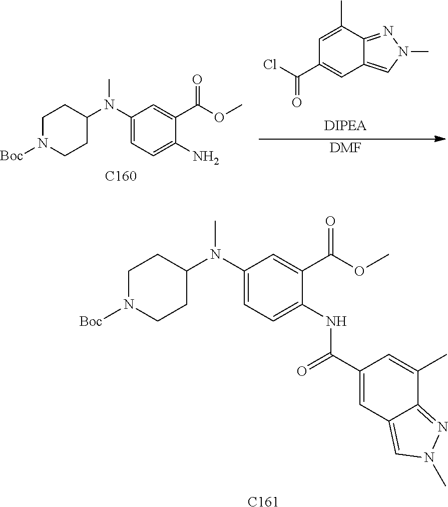

To a solution of tert-butyl 4-((4-amino-3-(methoxycarbonyl)phenyl)(methyl)amino)-piperidine-1-carboxylate (50.0 mg, 0.138 mmol) in DMF (728 uL) was added 2,7-dimethyl-2H-indazole-5-carbonyl chloride (37.3 mg, 0.179 mmol), followed by DIPEA (50.8 uL, 0.289 mmol). The reaction mixture was stirred at room temperature for 16 h then concentrated in vacuo to give a residue. The residue was partitioned between water (10 mL) and ethyl acetate (5 mL) The organic layer was separated, and the aqueous layer extracted with ethyl acetate (2×5 mL). The organic layers were combined, washed with saturated NaHCO₃, followed by brine, then dried over Na₂SO₄, and filtered. The filtrate was concentrated in vacuo to give a residue. The residue was purified by flash chromatography with a gradient of 0-75% of ethyl acetate in hexanes to provide tert-butyl 4-((4-(2,7-dimethyl-2H-indazole-5-carboxamido)-3-(methoxycarbonyl)phenyl)(methyl)amino)piperidine-1-carboxylate (48 mg, 65%). LCMS (ES, m/z): 536.3 [M+H]⁺.

### Synthesis of Intermediate C162

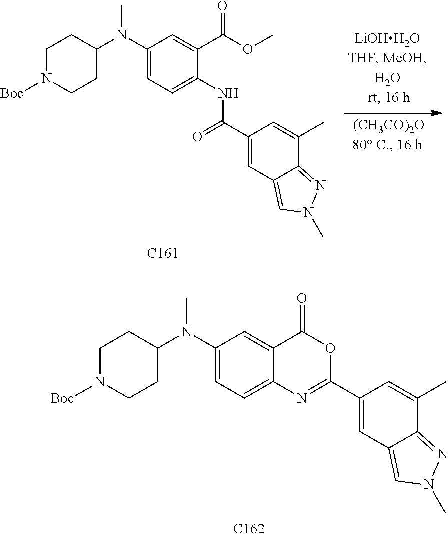

To a solution of tert-butyl 4-((4-(2,7-dimethyl-2H-indazole-5-carboxamido)-3-(methoxycarbonyl)phenyl)(methyl)amino)piperidine-1-carboxylate (50.0 mg, 0.99 mmol) in THF (625 uL) and methanol (625 uL) was added LiOH H₂O (8.4 mg, 0.20 mmol), followed by water (417 uL). The reaction mixture was stirred at room temperature for 16 h, then concentrated under reduced pressure and azeotroped with toluene to give a solid. The resulting solid was stirred in acetic anhydride (1.78 mL, 18.7 mmol) at 80° C. for 16 h. The reaction mixture was concentrated in vacuo to give a residue and the residue was stirred in diethyl ether for 1 h. A solid formed that was recovered by vacuum filtration and rinsed with diethyl ether to afford tert-butyl 4-((2-(2,7-dimethyl-2H-indazol-5-yl)-4-oxo-4H-benzo[d][1,3]oxazin-6-yl)(methyl)amino)piperidine-1-carboxylate (41.5 mg, 88%) as a solid. LCMS (ES, m/z): 504.3 [M+H]⁺.

### Synthesis of Intermediate C163

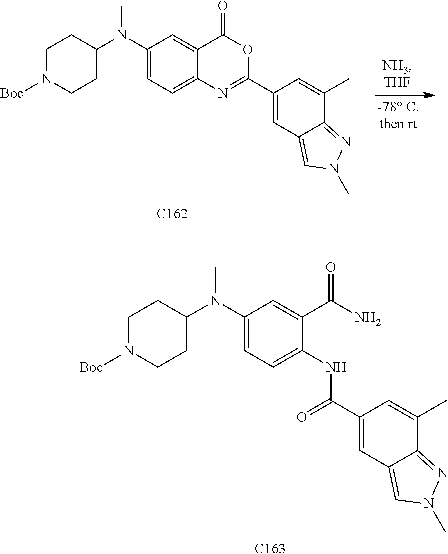

A solution of tert-butyl 4-((2-(2,7-dimethyl-2H-indazol-5-yl)-4-oxo-4H-benzo[d][1,3]oxazin-6-yl)(methyl)amino)piperidine-1-carboxylate (41.5 mg. 0.082 mmol) in THF (8 mL) was cooled to −78° C. and bubbled with NH₃ for 5 min. The reaction mixture was sealed and stirred for 16 h at room temperature. The resulting mixture was concentrated in vacuo to give a residue. The residue was purified by flash chromatography using a gradient of 0-5% methanol in DCM to provide tert-butyl 4-((3-carbamoyl-4-(2,7-dimethyl-2H-indazole-5-carboxamido)phenyl)(methyl)amino)piperidine-1-carboxylate (40.8 mg, 95%) as a solid. LCMS (ES, m/z): 521.1 [M+H]⁺.

### Synthesis of Intermediate C164

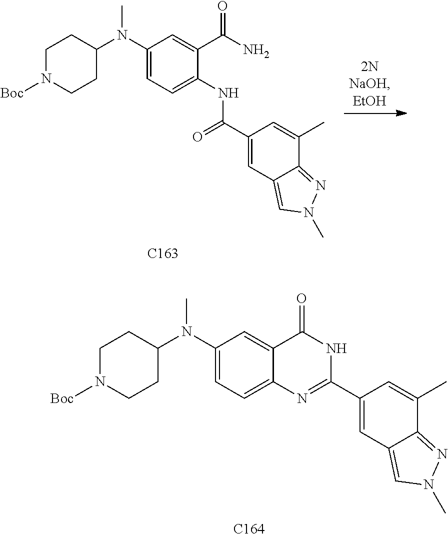

To a suspension of tert-butyl 4-((3-carbamoyl-4-(2,7-dimethyl-2H-indazole-5-carboxamido)phenyl)(methyl)amino)piperidine-1-carboxylate (105 mg, 0.20 mmol) in ethanol (5.00 mL was added 2N NaOH (0.9 mL, 1.82 mmol). The resulting solution was heated at 70° C. for 2 h, then concentrated under reduced pressure to give a solid. The solid was suspended in water (6 mL) and further dispersed by sonication for 5 min. The pH was adjusted to approximately 7 with 1N HCl and the suspension stirred for 2 h. The solid was collected by vacuum filtration, rinsed with water, and dried under high vacuum to afford tert-butyl 4-((2-(2,7-dimethyl-2H-indazol-5-yl)-4-oxo-3,4-dihydroquinazolin-6-yl)(methyl)amino)piperidine-1-carboxylate (95.5 mg, 95%) as a solid. LCMS (ES, m/z): 503.3 [M+H]⁺.

### Synthesis of Compound 121

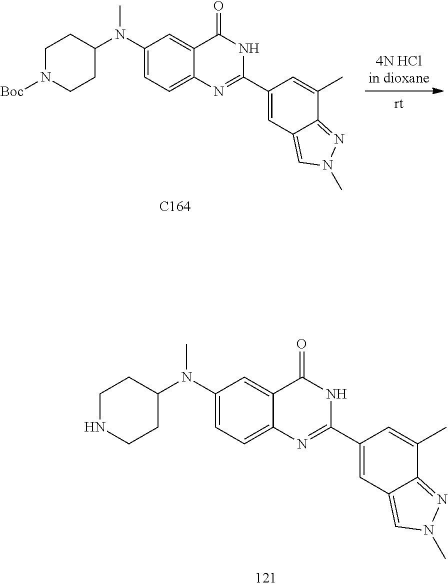 [SMILES: Cc1cc(-c2nc3ccc(N(C)C4CCN(C(=O)OC(C)(C)C)CC4)cc3c(=O)[nH]2)cc2cn(C)nc12 ; "C164" ; Cc1cc(-c2nc3ccc(N(C)C4CCNCC4)cc3c(=O)[nH]2)cc2cn(C)nc12]

To tert-butyl 4-((2-(2,7-dimethyl-2H-indazol-5-yl)-4-oxo-3,4-dihydroquinazolin-6-yl)(methyl)amino)piperidine-1-carboxylate (93.2 mg, 0.185 mmol) was added 4N HCl in dioxane (3.71 mL, 14.8 mmol) to form a suspension. The suspension was stirred for 1 h, then concentrated in vacuo to give a solid. The resulting solid was dissolved in water (2 mL). To the solution was added saturated NaHCO₃ until precipitation of a solid occurred. The solid was collected by filtration under reduced pressure and purified by reverse phase chromatography (Condition 1, Gradient 4). The fractions containing product were neutralized to pH 7 with NH₄OH (10%) by dropwise addition. The resulting solution was then lyophilized. The product was redissolved in water and lyophilised two additional times to yield 2-(2,7-dimethyl-2H-indazol-5-yl)-6-(methyl(piperidin-4-yl)amino)quinazolin-4(3H)-one (52 mg, 70%) as a solid. LCMS (ES, m/z): 403.3 [M+H]⁺. ¹H NMR (DMSO-d₆, 400 MHz): δ_{H} 12.21 (1H, br s), 8.49 (1H, s), 8.38 (1H, s), 7.84 (1H, s), 7.63 (1H, d, J=9.0 Hz), 7.49 (1H, dd, J=9.1, 3.0 Hz), 7.36 (1H, d, J=2.9 Hz), 4.19 (3H, s), 4.14-4.22 (1H, m), 3.36 (2H, d, J=12.7 Hz), 3.09 (2H, q, J=11.9 Hz), 2.82 (3H, s), 2.55 (3H, s), 1.90-2.01 (2H, m), 1.80 (2H, d, J=13.1 Hz).

### Example 166: Synthesis of compound 516

### Synthesis of Intermediate C165

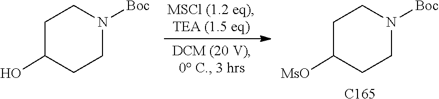 [SMILES: CC(C)(C)OC(=O)N1CCC(O)CC1 ; CC(C)(C)OC(=O)N1CCC(OS(C)(=O)=O)CC1 ; "C165"]

To a mixture of tert-butyl 4-hydroxypiperidine-1-carboxylate (20.0 g, 99.3 mmol, 1.00 eq) and triethylamine (15.0 g, 149 mmol, 1.50 eq) in dichloromethane (400 mL) was added methanesulfonyl chloride (13.6 g, 119 mmol, 1.20 eq) in portions at 0° C. under nitrogen atmosphere. The mixture was stirred at 0° C. for 3 hrs. The mixture was poured into saturated ammonium chloride (200 mL) and the resulting mixture was stirred for 0.5 hr at 20° C. The aqueous phase was extracted with ethyl acetate (3×200 mL). The combined organic phase was washed with brine (400 mL), dried with anhydrous Na₂SO₄, filtered and concentrated under reduce pressure. The crude product was triturated with methyl tert-butyl ether (400 mL). The solid was filtered and the filter cake was dried to give tert-butyl 4-methylsulfonyloxypiperidine-1-carboxylate (23.0 g, 83%) as a solid. ¹H NMR (400 MHz, CDCl₃-d) 6 ppm 4.87 (tt, J=7.46, 3.55 Hz, 1H) 3.63-3.75 (m, 2H) 3.24-3.35 (m, 2H) 3.03 (s, 3H) 1.90-1.99 (m, 2H) 1.76-1.87 (m, 2H) 1.45 (s, 9H)

### Synthesis of Intermediate C167

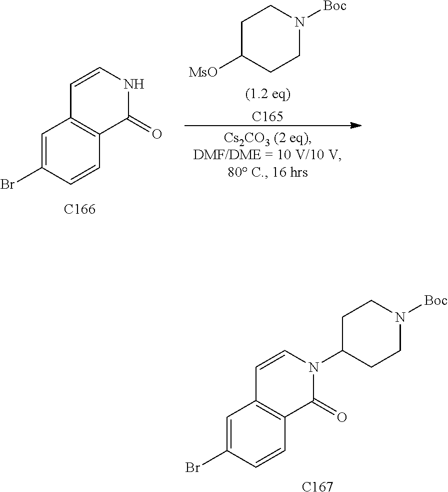 [SMILES: "C165" ; O=c1[nH]ccc2cc(Br)ccc12 ; CC(C)(C)OC(=O)N1CCC(OS(C)(=O)=O)CC1 ; "C166" ; CC(C)(C)OC(=O)N1CCC(n2ccc3cc(Br)ccc3c2=O)CC1 ; "C167"]

To a solution of 6-bromo-2H-isoquinolin-1-one (16.0 g, 71.4 mmol, 1 eq) and tert-butyl 4-methylsulfonyloxypiperidine-1-carboxylate (23.9 g, 85.6 mmol, 1.2 eq) in a mixture solvents of N,N-dimethylformamide (160 mL) and ethylene glycol dimethyl ether (160 mL) was added Cs₂CO₃ (46.5 g, 142 mmol, 2 eq) at 25° C. The reaction mixture was stirred at 80° C. for 16 hrs. The reaction mixture was quenched by ice water (100 mL) slowly and extracted with dichloromethane (3×300 mL). The combined organic phase was washed with brine (500 mL), dried over anhydrous Na₂SO₄, filtered and concentrated under reduced pressure. The residue was purified by column chromatography (petroleum ether/ethyl acetate=10/1 to 1/1) to give tert-butyl 4-(6-bromo-1-oxo-2-isoquinolyl) piperidine-1-carboxylate (10.0 g, 34%) as a solid. LCMS: (ESI, m/z):351.0, 353.0 [M-t-Bu]*. ¹H NMR (400 MHz, CDCl₃-d) 6 ppm 8.29 (d, J=8.80 Hz, 1H) 7.68 (d, J=1.96 Hz, 1H) 7.58 (dd, J=8.31, 1.96 Hz, 1H) 7.12 (d, J=7.34 Hz, 1H) 6.46 (d, J=7.34 Hz, 1H) 5.10-5.20 (m, 1H) 4.32 (br d, J=12.23 Hz, 2H) 2.93 (br t, J=12.47 Hz, 2H) 1.86-1.94 (m, 2H) 1.73 (qd, J=12.39, 3.91 Hz, 2H) 1.50 (s, 9H).

### Synthesis of Intermediate C168

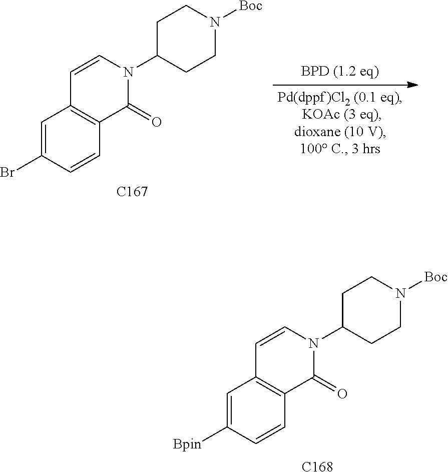

To a mixture of tert-butyl 4-(6-bromo-1-oxo-2-isoquinolyl)piperidine-1-carboxylate (25.0 g, 61.4 mmol, 1 eq) and 4,4,5,5-tetramethyl-2-(4,4,5,5-tetramethyl-1,3,2-dioxaborolan-2-yl)-1,3,2-dioxaborolane (18.7 g, 73.7 mmol, 1.2 eq) in dioxane (250 mL) was added [1,1-bis(diphenylphosphino) ferrocene]dichloropalladium(II) (4.49 g, 6.14 mmol, 0.1 eq) and acetoxypotassium (18.0 g, 184 mmol, 3 eq) at 20° C. under N₂ atmosphere. The mixture was stirred at 100° C. for 3 hrs. After cooling to 20° C., the mixture was filtered, and the filter cake was washed with dichloromethane (100 mL) and concentrated to give a residue. The residue was purified by silica gel chromatography (petroleum ether/ethyl acetate=3/1 to 1/1) to afford tert-butyl 4-[1-oxo-6-(4,4,5,5-tetramethyl-1,3,2-dioxaborolan-2-yl)-2-isoquinolyl]piperidine-1-carboxylate (30.0 g, 54%) as a solid. LCMS: (ESI, m/z): 399.2 [M-t-Bu]*. ¹H NMR (400 MHz, CDCl₃) δ ppm 8.42 (d, J=7.89 Hz, 1H) 7.98 (s, 1H) 7.88 (d, J=8.11 Hz, 1H) 7.09 (d, J=7.45 Hz, 1H) 6.57 (d, J=7.45 Hz, 1H) 5.20 (br s, 1H) 4.31 (br d, J=10.74 Hz, 2H) 2.93 (br t, J=12.50 Hz, 2H) 1.91 (br d, J=11.84 Hz, 2H) 1.74 (br dd, J=12.28, 3.29 Hz, 2H) 1.49 (s, 9H) 1.38 (s, 12H)

### Synthesis of Intermediate C171

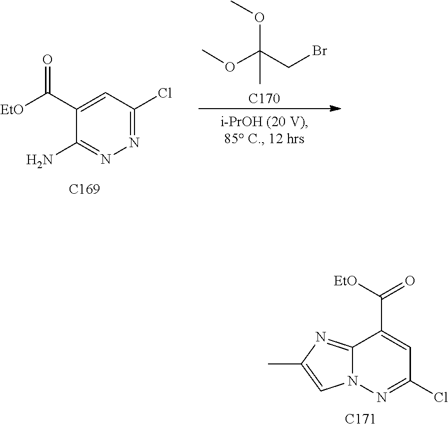

To a mixture of ethyl 3-amino-6-chloro-pyridazine-4-carboxylate (10.0 g, 49.6 mmol, 1 eq) in isopropanol (200 mL) was added 1-bromo-2,2-dimethoxy-propane (18.2 g, 99.2 mmol, 2 eq) at 20° C. The mixture was stirred at 85° C. for 12 hrs. After cooling to 20° C., the reaction mixture was concentrated to give a residue. Ice-water (50.0 mL) was added to the residue and the resulting mixture was extracted with ethyl acetate (3×20 mL). The combined organic phase was washed with brine (50.0 mL), dried over anhydrous Na₂SO₄, filtered and concentrated in vacuum. The residue was purified by silica gel chromatography (petroleum ether/ethyl acetate=3/1) to afford ethyl 6-chloro-2-methyl-imidazo[1,2-b]pyridazine-8-carboxylate (8.00 g, 67%) as a solid. LCMS: (ESI, m/z): 226.0 [M+H]⁺. ¹H NMR (400 MHz, CDCl₃) δ ppm 7.80 (s, 1H) 7.51 (s, 1H) 4.53 (q, J=7.15 Hz, 2H) 1.45 (t, J=7.15 Hz, 3H)

### Synthesis of Intermediate C172

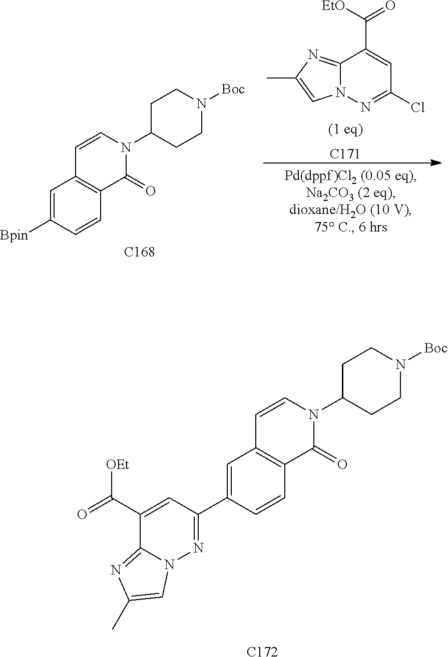

To a mixture of tert-butyl 4-[1-oxo-6-(4,4,5,5-tetramethyl-1,3,2-dioxaborolan-2-yl)-2-isoquinolyl]piperidine-1-carboxylate (6.00 g, 13.2 mmol, 1 eq) and ethyl 6-chloro-2-methyl-imidazo[1,2-b]pyridazine-8-carboxylate (3.16 g, 13.2 mmol, 1 eq) in dioxane (45.0 mL) and water (15.0 mL) was added [1,1-bis(diphenylphosphino)ferrocene]dichloropalladium(ii) (483 mg, 660 umol, 0.05 eq) and Na₂CO₃ (2.80 g, 26.4 mmol, 2 eq) at 20° C. under N₂ protection. The mixture was stirred at 75° C. for 6 hrs. After cooling to 25° C., the reaction mixture was poured into H₂O (20.0 mL) and extracted with ethyl acetate (3×10 mL). The combined organic phase was washed with brine (3×10 mL), dried with anhydrous Na₂SO₄, filtered and concentrated to give crude product. The reaction mixture was concentrated under reduce pressure to give a residue, which was purified by silica gel chromatography (petroleum ether/ethyl acetate=1/1) to give crude product. The crude product was purified by prep-HPLC (Condition 12, Gradient 1) to give ethyl 6-[2-(1-tert-butoxycarbonyl-4-piperidyl)-1-oxo-6-isoquinolyl]-2-methyl-imidazo[1,2-b]pyridazine-8-carboxylate (2.30 g, 33%) as a solid. LCMS: (ESI, m/z): 504.3 [M+H]⁺. ¹H NMR (400 MHz, DMSO-d₆) δ ppm 8.34-8.41 (m, 2H) 8.30 (s, 1H) 8.14-8.19 (m, 2H) 7.64 (d, J=7.34 Hz, 1H) 6.80 (d, J=7.82 Hz, 1H) 4.94-5.05 (m, 1H) 4.48 (q, J=7.34 Hz, 2H) 4.09-4.18 (m, 2H) 2.92 (br d, J=3.42 Hz, 2H) 1.77-1.85 (m, 4H) 1.44 (s, 9H) 1.40 (t, J=7.09 Hz, 3H)

### Synthesis of Intermediate C173

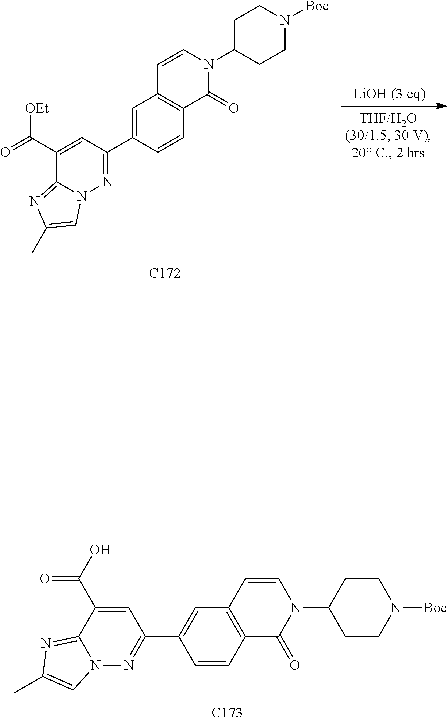 [SMILES: CCOC(=O)c1cc(-c2ccc3c(=O)n(C4CCN(C(=O)OC(C)(C)C)CC4)ccc3c2)nn2cc(C)nc12 ; "C172" ; Cc1cn2nc(-c3ccc4c(=O)n(C5CCN(C(=O)OC(C)(C)C)CC5)ccc4c3)cc(C(=O)O)c2n1 ; "C173"]

To a mixture of ethyl 6-[2-(1-tert-butoxycarbonyl-4-piperidyl)-1-oxo-6-isoquinolyl]-2-methyl-imidazo[1,2-b]pyridazine-8-carboxylate (1.00 g, 1.88 mmol, 1 eq) in a mixture of tetrahydrofuran (30.0 mL) and H₂O (1.50 mL) was added lithium hydroxide monohydrate (237 mg, 5.64 mmol, 3 eq) at 20° C. The reaction mixture was stirred at 20° C. for 2 hrs and adjusted to pH 5-6 with 1N HCl. The mixture was filtered and the filter cake was dried to give 6-[2-(1-tert-butoxycarbonyl-4-piperidyl)-1-oxo-6-isoquinolyl]-2-methyl-imidazo[1,2-b]pyridazine-8-carboxylic acid (600 mg, 63%) as a solid. LCMS: (ESI, m/z): 504.3 [M+H]⁺, ¹H NMR (DMSO-d₆, 400 MHz) δ 8.3-8.4 (m, 2H), 8.0-8.2 (m, 3H), 7.64 (d, 1H, J=7.6 Hz), 6.84 (d, 1H, J=7.6 Hz), 4.9-5.1 (m, 1H), 4.1-4.2 (m, 2H), 2.8-3.0 (m, 2H), 2.42 (s, 3H), 1.7-1.9 (m, 4H), 1.44 (s, 9H)

### Synthesis of Compound 516

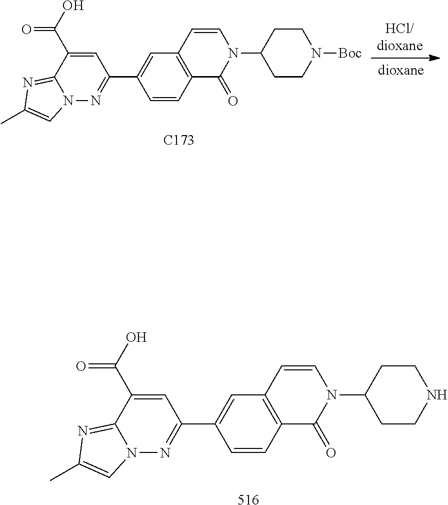 [SMILES: Cc1cn2nc(-c3ccc4c(=O)n(C5CCN(C(=O)OC(C)(C)C)CC5)ccc4c3)cc(C(=O)O)c2n1 ; "C173" ; Cc1cn2nc(-c3ccc4c(=O)n(C5CCNCC5)ccc4c3)cc(C(=O)O)c2n1 ; "516"]

To a mixture of 6-[2-(1-tert-butoxycarbonyl-4-piperidyl)-1-oxo-6-isoquinolyl]-2-methyl-imidazo[1,2-b]pyridazine-8-carboxylic acid (150 mg, 298 umol, 1 eq) in dioxane (3.00 mL) was added HCl/dioxane (4 M, 3.00 mL). The mixture was stirred at 20° C. for 1 hr. TLC (ethyl acetate/methanol=3/1, Rf=0.05) showed all the starting material was consumed and a new major spot was generated. The mixture was filtered and the filter cake was dried to give 2-methyl-6-[1-oxo-2-(4-piperidyl)-6-isoquinolyl]imidazo[1,2-b]pyridazine-8-carboxylic acid (101 mg, 77%) as a solid. LCMS: (ESI, m/z): 404.1 [M+H]⁺, ¹H NMR (MeOD-d₄, 400 MHz) δ 8.84 (s, 1H), 8.55 (d, 1H, J=8.5 Hz), 8.4-8.5 (m, 2H), 8.32 (dd, 1H, J=1.8, 8.5 Hz), 7.51 (d, 1H, J=7.6 Hz), 6.95 (d, 1H, J=7.5 Hz), 5.1-5.2 (m, 1H), 3.63 (br d, 2H, J=13.1 Hz), 3.27 (br s, 2H), 2.70 (s, 3H), 2.3-2.4 (m, 2H), 2.19 (br d, 2H, J=13.5 Hz)

### Example 167: Synthesis of Compound 517

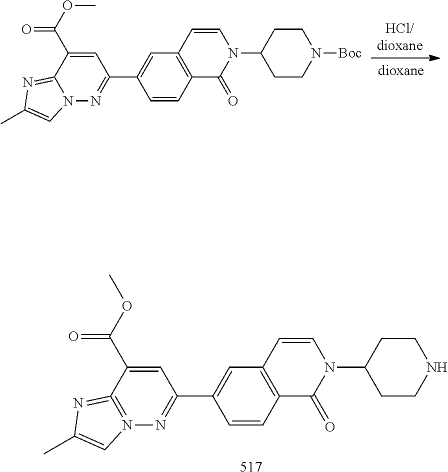

To a mixture of methyl 6-[2-(1-tert-butoxycarbonyl-4-piperidyl)-1-oxo-6-isoquinolyl]-2-methyl-imidazo[1,2-b]pyridazine-8-carboxylate (80.0 mg, 155 umol, 1 eq) in dioxane (3.00 mL) was added HCl/dioxane (4 M, 3.00 mL). The mixture was stirred at 20° C. for 1 hr. The solid was filtered, washed with CH₂Cl₂ (3×2 mL) and dried to give methyl 2-methyl-6-[1-oxo-2-(4-piperidyl)-6-isoquinolyl]imidazo[1,2-b]pyridazine-8-carboxylate (67.3 mg, 96%) as a solid. LCMS: (ESI, m/z): 418.1 [M+H]⁺, ¹H NMR (MeOD-d₄, 400 MHz) δ 8.93 (s, 1H), 8.5-8.6 (m, 3H), 8.33 (dd, 1H, J=1.8, 8.6 Hz), 7.54 (d, 1H, J=7.5 Hz), 6.96 (d, 1H, J=7.5 Hz), 5.1-5.2 (m, 1H), 4.21 (s, 3H), 3.63 (br d, 2H, J=13.0 Hz), 3.30 (br s, 2H), 2.73 (d, 3H, J=0.7 Hz), 2.3-2.5 (m, 2H), 2.18 (br d, 2H, J=13.5 Hz)

### Example 168: Synthesis of Compound 518

### Synthesis of Intermediate C174

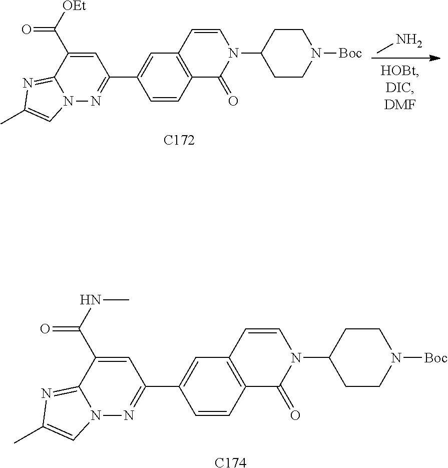

A mixture of ethyl 6-[2-(1-tert-butoxycarbonyl-4-piperidyl)-1-oxo-6-isoquinolyl]-2-methyl-imidazo[1,2-b]pyridazine-8-carboxylate (146 mg, 282 umol, 1 eq), 1-hydroxybenzotriazole (95.3 mg, 705 umol, 2.5 eq), and N,N′-di(propan-2-yl)methanediimine (89.0 mg, 705 umol, 109 μL, 2.5 eq) in DMF (1.50 mL) was added MeNH₂ (2 M in THF, 2.82 mL, 20 eq) at 25° C. The mixture was stirred at 105° C. for 12 hrs under N₂ atmosphere. The reaction mixture was quenched by H₂O (2.00 mL) at 25° C. The mixture was filtered and the filter cake was washed with ethyl acetate (2.00 mL) and dried in vacuum to give tert-butyl 4-[6-[2-methyl-8-(methylcarbamoyl) imidazo[1,2-b]pyridazin-6-yl]-1-oxo-2-isoquino lyl]piperidine-1-carboxylate (100 mg, 62%) as a solid. LCMS: (ESI, m/z): 517.3 [M+H]^{+ 1}H NMR (CDCl₃, 400 MHz) δ 9.66 (br d, 1H, J=5.0 Hz), 8.3-8.5 (m, 4H), 8.19 (dd, 1H, J=1.1, 8.5 Hz), 7.65 (d, 1H, J=7.8 Hz), 6.86 (d, 1H, J=7.4 Hz), 4.8-5.2 (m, 1H), 3.9-4.3 (m, 2H), 2.8-3.1 (m, 6H), 1.7-1.9 (m, 4H), 1.44 (s, 9H)

### Synthesis of Compound 518

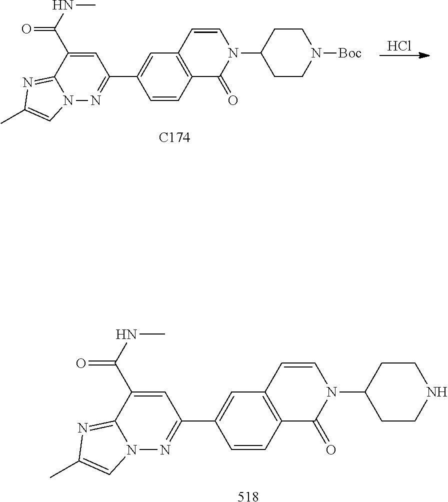 [SMILES: CNC(=O)c1cc(-c2ccc3c(=O)n(C4CCN(C(=O)OC(C)(C)C)CC4)ccc3c2)nn2cc(C)nc12 ; Cl ; "C174" ; CNC(=O)c1cc(-c2ccc3c(=O)n(C4CCNCC4)ccc3c2)nn2cc(C)nc12 ; "518"]

A mixture of tert-butyl 4-[6-[2-methyl-8-(methylcarbamoyl)imidazo[1,2-b]pyridazin-6-yl]-1-oxo-2-isoquinolyl]piperidine-1-carboxylate (EVAL-0116-0004a, 100 mg, 193 umol, 1 eq) in HCl/ethyl acetate (4 M, 3.00 mL) was stirred at 20° C. for 1 hr. TLC (ethyl acetate, Rf=0.1) showed the starting material was consumed and a new major spot was generated. The mixture was filtered and the solid was collected to give N, 2-dimethyl-6-[1-oxo-2-(4-piperidyl)-6-isoquinolyl]imidazo[1,2-b]pyridazine-8-carboxamide (49.9 mg, 57%) a solid. LCMS: (ESI, m/z): 417.1 [M+H]^{+ 1}H NMR (MeOD-d₄, 400 MHz) δ 8.84 (s, 1H), 8.5-8.6 (m, 2H), 8.46 (d, 1H, J=1.0 Hz), 8.33 (dd, 1H, J=1.7, 8.6 Hz), 7.54 (d, 1H, J=7.5 Hz), 6.94 (d, 1H, J=7.4 Hz), 5.1-5.2 (m, 1H), 3.63 (br d, 2H, J=12.8 Hz), 3.26 (br d, 2H, J=2.6 Hz), 3.09 (s, 3H), 2.69 (s, 3H), 2.36 (dq, 2H, J=3.9, 13.0 Hz), 2.1-2.3 (m, 2H)

### Example 169: Synthesis of Compound 523

### Synthesis of Intermediate C175

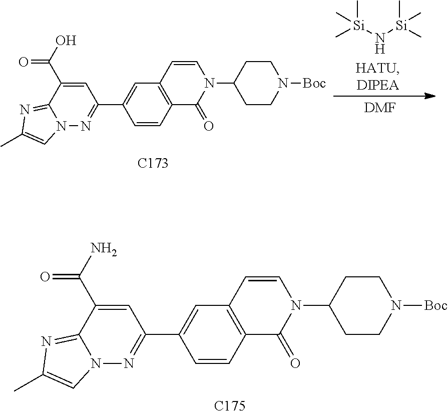

To a mixture of 6-[2-(1-tert-butoxycarbonyl-4-piperidyl)-1-oxo-6-isoquinolyl]-2-methyl-imidazo[1,2-b]pyridazine-8-carboxylic acid (100 mg, 199 umol, 1 eq), 0-(7-Azabenzotriazol-1-yl)-N,N,N′,N′-tetramethyluronium hexafluoro-phosphate (98.2 mg, 258 umol, 1.3 eq) and diisopropylethylamine (128 mg, 993 umol, 173 μL, 5 eq) in N,N-dimethylformamide (3.00 mL) was added [dimethyl-(trimethylsilylamino)silyl]methane (41.7 mg, 258 umol, 54.1 uL, 1.3 eq) at 20° C. The mixture was stirred at 20° C. for 16 hrs. The reaction mixture was poured into ice water (20 mL) and the resulting mixture was extracted with CH₂Cl₂ (4×10 mL). The combined organic phase was dried with anhydrous Na₂SO₄, filtered and concentrated in vacuum to give a residue, which was purified by silica gel chromatography (petroleum ether/Ethyl acetate=3/1) to give tert-butyl 4-[6-(8-carbamoyl-2-methyl-imidazo[1,2-b]pyridazin-6-yl)-1-oxo-2-isoquinolyl]piperidine-1-carboxylate (40.0 mg, 40%) as a solid. LCMS: (ESI, m/z): 503.3 [M+H]⁺, ¹H NMR (DMSO-d₆, 400 MHz) δ 9.20 (br d, 1H, J=2.0 Hz), 8.4-8.5 (m, 2H), 8.39 (d, 1H, J=8.6 Hz), 8.34 (s, 1H), 8.29 (s, 1H), 8.19 (dd, 1H, J=1.3, 8.5 Hz), 7.65 (d, 1H, J=7.6 Hz), 6.86 (d, 1H, J=7.5 Hz), 4.9-5.1 (m, 1H), 4.1-4.2 (m, 2H), 2.9-3.0 (m, 2H), 2.5-2.5 (m, 3H), 1.7-1.9 (m, 4H), 1.44 (s, 9H)

### Synthesis of Compound 523

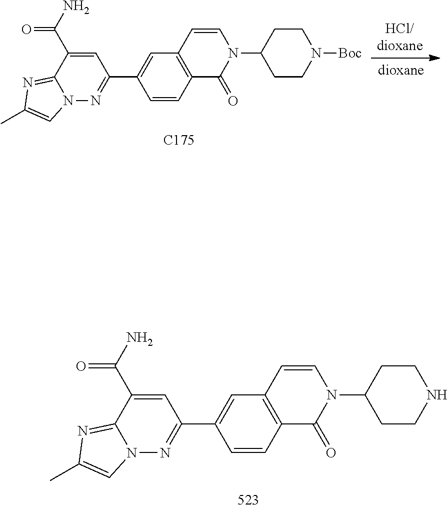 [SMILES: Cc1cn2nc(-c3ccc4c(=O)n(C5CCN(C(=O)OC(C)(C)C)CC5)ccc4c3)cc(C(N)=O)c2n1 ; "C175" ; Cc1cn2nc(-c3ccc4c(=O)n(C5CCNCC5)ccc4c3)cc(C(N)=O)c2n1 ; "523"]

To a mixture of tert-butyl 4-[6-(8-carbamoyl-2-methyl-imidazo[1,2-b]pyridazin-6-yl)-1-oxo-2-isoquinolyl]piperidine-1-carboxylate (40.0 mg, 79.6 umol, 1 eq) in dioxane (3.00 mL) was added HCl/dioxane (4 M, 3.00 mL). The mixture was stirred at 20° C. for 1 hr. TLC (ethyl acetate, Rf=0) showed all of the starting material was consumed and a new major spot was generated. The solid was filtered, washed with CH₂Cl₂ (3×2 mL) and dried to give 2-methyl-6-[1-oxo-2-(4-piperidyl)-6-isoquinolyl]imidazo[1,2-b]pyridazine-8-carboxamide (24.8 mg, 45%) as a solid. LCMS: (ESI, m/z): 403.1 [M+H]⁺, ¹H NMR (METHANOL-d4, 400 MHz) δ 8.89 (s, 1H), 8.55 (d, 1H, J=8.6 Hz), 8.51 (d, 1H, J=1.4 Hz), 8.45 (s, 1H), 8.33 (dd, 1H, J=1.7, 8.6 Hz), 7.53 (d, 1H, J=7.6 Hz), 6.94 (d, 1H, J=7.5 Hz), 5.1-5.2 (m, 1H), 3.63 (br d, 2H, J=13.2 Hz), 3.27 (br s, 2H), 2.69 (s, 3H), 2.36 (dq, 2H, J=3.7, 13.0 Hz), 2.1-2.2 (m, 2H)

### Example 170: Synthesis of Compound 524

### Synthesis of Intermediate C176

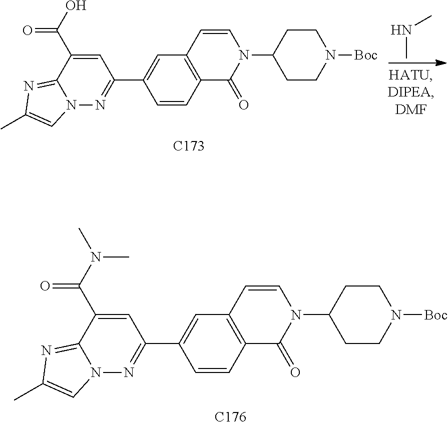

A mixture of 6-(2-(1-(tert-butoxycarbonyl)piperidin-4-yl)-1-oxo-1,2-dihydroisoquinolin-6-yl)-2-methylimidazo[1,2-b]pyridazine-8-carboxylic acid (200 mg, 376 umol, 1 eq), N,N-diisopropylethylamine (146 mg, 1.13 mmol, 197 μL, 3 eq), o-(7-Azabenzotriazol-1-yl)-N,N,N′,N′-tetramethyluronium hexafluorophosphate (114 mg, 1.13 mmol, 157 μL, 3 eq) in N,N-dimethylformamide (2.00 mL) was N-methylmethanamine; hydrochloride (153 mg, 1.88 mmol, 5 eq). The mixture was stirred at 80° C. for 16 hrs under N₂ atmosphere. After cooling to 20° C., the reaction mixture was quenched with water (15.0 mL) at 25° C. and extracted with ethyl acetate (3×6 mL). The combined organic layer was dried over Na₂SO₄, filtered and the filtrate was concentrated under reduced pressure to give crude product, which was purified by column chromatography on silica gel (94% of ethyl acetate in petroleum ether) to give tert-butyl 4-[6-[8-(dimethylcarbamoyl)-2-methyl-imidazo[1,2-b]pyridazin-6-yl]-1-oxo-2-isoquinolyl]piperidine-1-carboxylate (50.0 mg, 25%) as a solid. LCMS: (ESI, m/z): 531.2 [M+H]⁺, ¹H NMR (400 MHz, DMSO-d₆) δ ppm 8.42 (d, J=1.59 Hz, 1H) 8.37 (d, J=8.44 Hz, 1H) 8.25 (d, J=0.61 Hz, 1H) 8.20 (dd, J=8.56, 1.71 Hz, 1H) 7.89 (s, 1H) 7.64 (d, J=7.70 Hz, 1H) 6.76 (d, J=7.58 Hz, 1H) 4.94-5.04 (m, 1H) 3.10 (s, 3H) 2.89 (s, 3H) 2.86 (s, 4H) 2.44 (s, 3H) 1.74-1.85 (m, 4H) 1.44 (s, 9H)

### Synthesis of Compound 524

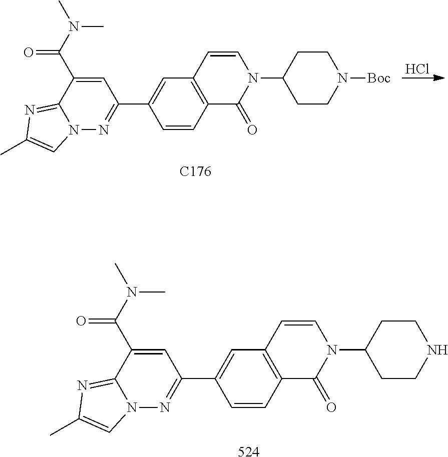 [SMILES: Cc1cn2nc(-c3ccc4c(=O)n(C5CCN(C(=O)OC(C)(C)C)CC5)ccc4c3)cc(C(=O)N(C)C)c2n1 ; Cl ; "C176" ; Cc1cn2nc(-c3ccc4c(=O)n(C5CCNCC5)ccc4c3)cc(C(=O)N(C)C)c2n1 ; "524"]

To a mixture of tert-butyl 4-[6-[8-(dimethylcarbamoyl)-2-methyl-imidazo[1,2-b]pyridazin-6-yl]-1-oxo-2-isoquinolyl]piperidine-1-carboxylate (50.0 mg, 94.2 umol, 1 eq) in ethyl acetate (100 uL) was added HCl/ethyl acetate (4 M, 500 uL). The mixture was stirred at 25° C. for 1 hr. The reaction mixture was filtered and the filter was concentrated to give a residue. The crude product was triturated with tert-butyl methyl ether (2.00 mL). The mixture was filtered and the filter cake was dried to give N, N, 2-trimethyl-6-[1-oxo-2-(4-piperidyl)-6-isoquinolyl]imidazo[1,2-b]pyridazine-8-carboxamide (10 mg, 30%) as a solid. LCMS: (ESI, m/z): 431.2 [M+H]⁺¹H NMR (400 MHz, MeOD-d₄) δ ppm 8.50-8.56 (m, 2H) 8.45 (dd, J=6.48, 1.22 Hz, 2H) 8.30 (dd, J=8.56, 1.71 Hz, 1H) 7.50 (d, J=7.70 Hz, 1H) 6.93 (d, J=7.58 Hz, 1H) 5.10-5.18 (m, 1H) 3.62 (br d, J=12.59 Hz, 2H) 3.27-3.30 (m, 2H) 3.27 (s, 3H) 3.15 (s, 3H) 2.66 (d, J=0.61 Hz, 3H) 2.33 (qd, J=12.90, 4.22 Hz, 2H) 2.14-2.23 (m, 2H).

### Example 170: Synthesis of Compound 527

### Synthesis of Intermediate C178

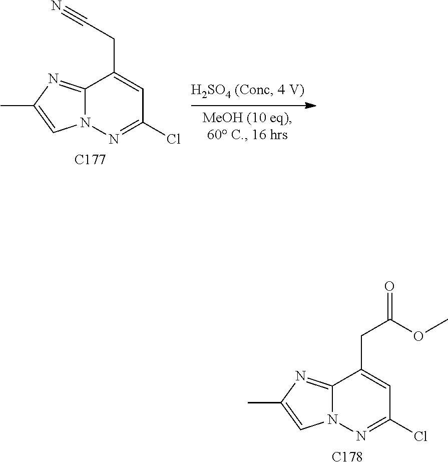

To a mixture of 2-(6-chloro-2-methyl-imidazo[1,2-b]pyridazin-8-yl)acetonitrile (2.00 g, 9.68 mmol, 1 eq) in concentrated sulfuric acid (2.00 mL) was added methanol (3.10 g, 96.7 mmol, 10 eq) at 20° C. The mixture was stirred at 60° C. for 16 hrs. After cooled to 25° C., filtered and concentrated in vacuum. The residue was purified by silica gel chromatography (petroleum ether/ethyl acetate=1/1) to afford methyl 2-(6-chloro-2-methyl-imidazo[1,2-b]pyridazin-8-yl)acetate (1.40 g, 60%) as a solid. LCMS: (ESI, m/z): 240.0 [M+H]⁺. ¹H NMR (400 MHz, CDCl₃) δ ppm 7.71 (s, 1H) 7.04 (s, 1H) 4.07 (s, 2H) 3.77 (s, 3H) 2.50 (s, 3H)

### Synthesis of Intermediate C179

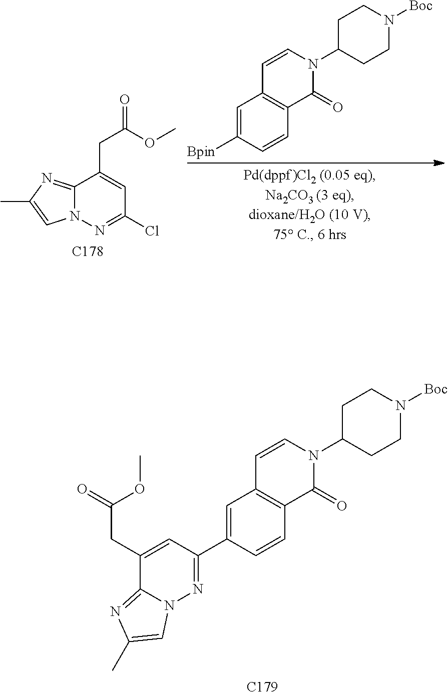

To a mixture of methyl 2-(6-chloro-2-methyl-imidazo[1,2-b]pyridazin-8-yl)acetate (700 mg, 2.92 mmol, 1 eq) and tert-butyl 4-[1-oxo-6-(4,4,5,5-tetramethyl-1,3,2-dioxaborolan-2-yl)-2-isoquinolyl]piperidine-1-carboxylate (1.33 g, 2.92 mmol, 1 eq) in dioxane (7.00 mL) and water (1.00 mL) was added [1,1 bis(diphenylphosphino)ferrocene]dichloropalladium(II) (213 mg, 292 umol, 0.1 eq) and Na₂CO₃ (618 mg, 5.84 mmol, 2 eq) in portions at 20° C. under N₂ protection. The mixture was stirred at 75° C. for 6 hrs. After cooling to 20° C., the reaction mixture was concentrated to give a residue, which was purified by silica gel chromatography (petroleum ether/ethyl acetate=1/1) to afford tert-butyl 4-[6-[8-(2-methoxy-2-oxo-ethyl)-2-methyl-imidazo[1,2-b]pyridazin-6-yl]-1-oxo-2-isoquinolyl]piperidine-1-carboxylate (800 mg, 52%) as a solid. LCMS: (ESI, m/z): 532.1 [M+H]⁺. ¹H NMR (400 MHz, MeOD-d₄) δ ppm 8.44 (d, J=8.31 Hz, 1H) 8.25 (d, J=1.96 Hz, 1H) 8.16 (dd, J=8.56, 1.71 Hz, 1H) 7.98 (s, 1H) 7.79 (s, 1H) 7.50 (d, J=7.82 Hz, 1H) 6.83 (d, J=7.34 Hz, 1H) 5.05-5.14 (m, 1H) 4.31 (br d, J=13.20 Hz, 2H) 4.16 (s, 2H) 3.76 (s, 3H) 2.98 (br d, J=11.74 Hz, 2H) 2.49 (s, 3H) 1.87-1.93 (m, 4H) 1.50 (s, 9H)

### Synthesis of Compound 527

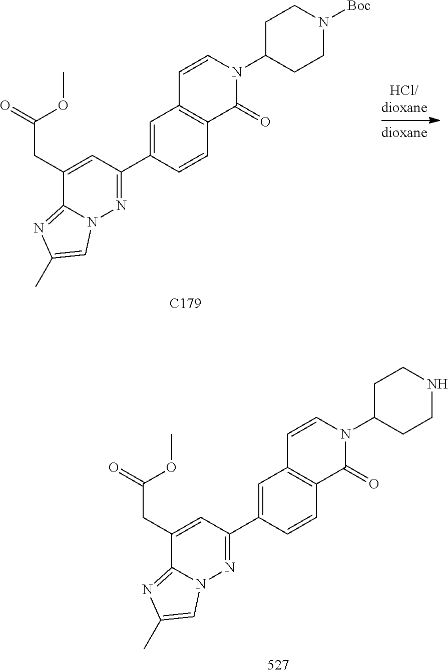 [SMILES: COC(=O)Cc1cc(-c2ccc3c(=O)n(C4CCN(C(=O)OC(C)(C)C)CC4)ccc3c2)nn2cc(C)nc12 ; "C179" ; COC(=O)Cc1cc(-c2ccc3c(=O)n(C4CCNCC4)ccc3c2)nn2cc(C)nc12 ; "527"]

To a solution of tert-butyl 4-[6-[8-(2-methoxy-2-oxo-ethyl)-2-methyl-imidazo[1,2-b]pyridazin-6-yl]-1-oxo-2-isoquinolyl]piperidine-1-carboxylate (50.0 mg, 94.0 umol, 1 eq) in dioxane (1.00 mL) was added HCl/dioxane (2 M, 1.00 mL) at 20° C. The mixture was stirred at 20° C. for 2 hrs. The mixture was concentrated to give a residue, which was triturated with methyl tert-butyl ether (3 mL). The solid was filtered and the filter cake was dried to give 2-[2-methyl-6-[1-oxo-2-(4-piperidyl)-6-isoquinolyl]imidazo[1,2-b]pyridazin-8-yl]acetate (25.0 mg, 60%) as a solid. LCMS: (ESI, m/z): 432.2 [M+H]⁺. ¹H NMR (400 MHz, MeOD-d₄) δ ppm 8.54 (d, J=8.50 Hz, 1H) 8.38-8.46 (m, 3H) 8.28 (dd, J=8.51, 1.50 Hz, 1H) 7.50 (d, J=7.50 Hz, 1H) 6.93 (d, J=7.50 Hz, 1H) 5.15 (br s, 1H) 4.31 (s, 2H) 3.82 (s, 3H) 3.63 (br d, J=13.01 Hz, 2H) 3.24-3.29 (m, 2H) 2.67 (s, 3H) 2.34 (br dd, J=12.51, 3.50 Hz, 2H) 2.15-2.24 (m, 2H)

### Example 171: Synthesis of Compound 528

### Synthesis of Intermediate C180

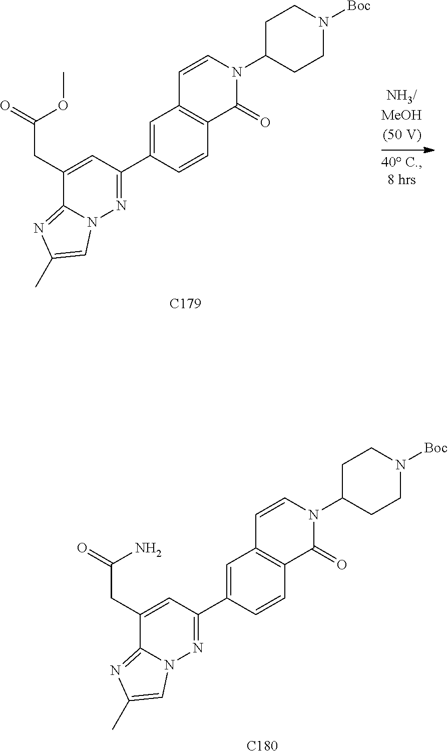 [SMILES: COC(=O)Cc1cc(-c2ccc3c(=O)n(C4CCN(C(=O)OC(C)(C)C)CC4)ccc3c2)nn2cc(C)nc12 ; "C179" ; Cc1cn2nc(-c3ccc4c(=O)n(C5CCN(C(=O)OC(C)(C)C)CC5)ccc4c3)cc(CC(N)=O)c2n1 ; "C180"]

A mixture of tert-butyl 4-(6-(8-(2-methoxy-2-oxoethyl)-2-methylimidazo[1,2-b]pyridazin-6-yl)-1-oxoisoquinolin-2(1H)-yl) piperidine-1-carboxylate (80.0 mg, 150 umol, 1 eq) in NH₃/MeOH (6 M, 4.00 mL) was stirred for 8 hrs at 40° C. The reaction mixture was concentrated. The crude product was triturated with methyl tert-butyl ether at 25° C. for 60 min to obtained tert-butyl 4-(6-(8-(2-amino-2-oxoethyl)-2-methylimidazo[1,2-b]pyridazin-6-yl)-1-oxoisoquinolin-2(1H)-yl) piperidine-1-carboxylate (70.0 mg, 77%) as a solid. LCMS: (ESI, m/z): 517.2 [M+H]⁺. ¹HNMR (400 MHz, CDCl₃) δ ppm 8.56 (br d, J=7.9 Hz, 1H) 8.15-8.00 (m, 2H) 7.83 (s, 1H) 7.55 (br s, 1H) 7.17 (br d, J=6.7 Hz, 1H) 6.65 (br d, J=6.7 Hz, 1H) 5.53 (br s, 1H) 5.29-5.17 (m, 1H), 5.00 (spt, J=6.3 Hz, 1H) 4.47-4.20 (m, 2H) 4.03 (br s, 2H) 2.95 (br d, J=1.2 Hz, 2H) 2.54 (s, 3H) 1.77 (br d, J=9.5 Hz, 2H) 1.50 (s, 9H).

### Synthesis of Compound 528

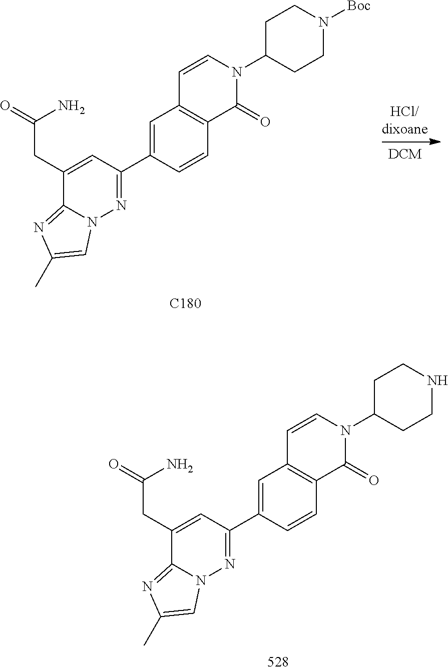 [SMILES: Cc1cn2nc(-c3ccc4c(=O)n(C5CCN(C(=O)OC(C)(C)C)CC5)ccc4c3)cc(CC(N)=O)c2n1 ; "C180" ; Cc1cn2nc(-c3ccc4c(=O)n(C5CCNCC5)ccc4c3)cc(CC(N)=O)c2n1 ; "528"]

A solution of tert-butyl 4-(6-(8-(2-amino-2-oxoethyl)-2-methylimidazo[1,2-b]pyridazin-6-yl)-1-oxoisoquinolin-2(1H)-yl)piperidine-1-carboxylate (70.0 mg, 135 umol, 1 eq) in DCM (1.00 mL) was added HCl/dioxane (2 M, 900 μL, 13.3 eq) and the resulting mixture was stirred for 2 hrs at 25° C. LCMS showed the starting material was consumed completely and one major peak with desired MS was detected. The reaction mixture was filtered. The solid was collected and purified by prep-HPLC (Condition 13, Gradient 1) to afford 2-(2-methyl-6-(1-oxo-2-(piperidin-4-yl)-1,2-dihydroisoquinolin-6-yl)imidazo[1,2-b]pyridazin-8-yl) acetamide (20.3 mg, 32%) as a solid. LCMS: (ESI, m/z): 417.1 [M+H]⁺. ¹HNMR (400 MHz, CD₃OD) δ ppm 8.52 (d, J=8.6 Hz, 1H) 8.45-8.41 (m, 2H) 8.39 (s, 1H) 8.27 (dd, J=1.7, 8.6 Hz, 1H) 7.53 (d, J=7.6 Hz, 1H) 6.93 (d, J=7.6 Hz, 1H) 5.17 (tt, J=3.8, 12.2 Hz, 1H) 4.22 (s, 2H) 3.63 (br d, J=13.0 Hz, 2H) 3.31-3.26 (m, 2H) 2.67 (s, 3H) 2.37 (dq, J=4.1, 13.0 Hz, 2H) 2.18 (br d, J=13.4 Hz, 2H)

### Example 172: Synthesis of Compound 531

### Synthesis of Intermediate C181

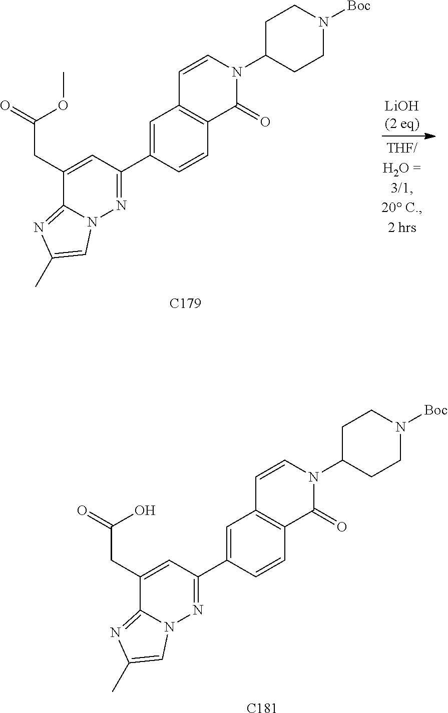 [SMILES: COC(=O)Cc1cc(-c2ccc3c(=O)n(C4CCN(C(=O)OC(C)(C)C)CC4)ccc3c2)nn2cc(C)nc12 ; "C179" ; Cc1cn2nc(-c3ccc4c(=O)n(C5CCN(C(=O)OC(C)(C)C)CC5)ccc4c3)cc(CC(=O)O)c2n1 ; "C181"]

To a mixture of tert-butyl 4-[6-[8-(2-methoxy-2-oxo-ethyl)-2-methyl-imidazo[1,2-b]pyridazin-6-yl]-1-oxo-2-isoquinolyl]piperidine-1-carboxylate (103 mg, 193 umol, 1 eq) in tetrahydrofuran (3.00 mL) and water (1.00 mL) was added Lithium hydroxide monohydrate (16.2 mg, 386 umol, 2 eq) in one portion at 20° C. The mixture was stirred at 20° C. for 2 hrs. The mixture was concentrated in reduced pressure at 45° C. The residue was added ice-water (w/w=1/1) (5 mL) and stirred for 30 min. The aqueous phase was adjusted to pH=2 with 1N HCl. The mixture was filtrated and dried in vacuo. The crude product was triturated with methyl tert-butyl ether (3 mL) to afford 2-[6-[2-(1-tert-butoxycarbonyl-4-piperidyl)-1-oxo-6-isoquinolyl]-2-methyl-imidazo[1,2-b]pyridazin-8-yl]acetic acid (70.0 mg, 70%) as a solid. LCMS: (ESI, m/z): 518.2 [M+H]⁺. ¹H NMR (400 MHz, MeOD-d4) δ ppm 8.40 (d, J=8.51 Hz, 1H) 8.23 (s, 1H) 8.13 (br d, J=8.50 Hz, 1H) 7.94-8.07 (m, 2H) 7.50 (d, J=7.00 Hz, 1H) 6.80 (br d, J=7.50 Hz, 1H) 5.08 (br t, J=7.75 Hz, 1H) 4.31 (br d, J=13.01 Hz, 2H) 2.99 (br s, 2H) 2.52 (s, 3H) 1.84-1.94 (m, 4H) 1.50 (s, 9H)

### Synthesis of Compound 531

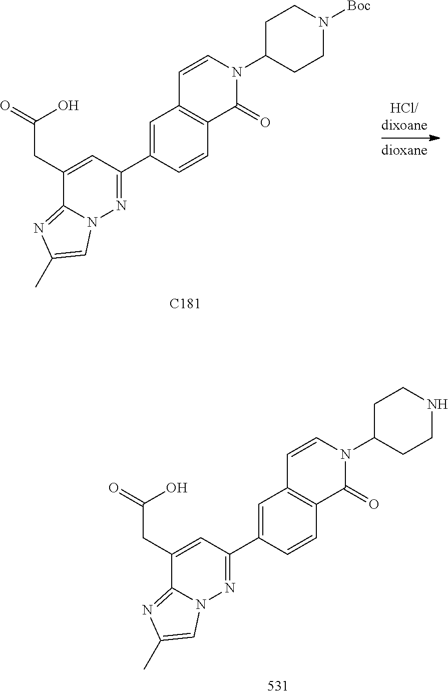 [SMILES: Cc1cn2nc(-c3ccc4c(=O)n(C5CCN(C(=O)OC(C)(C)C)CC5)ccc4c3)cc(CC(=O)O)c2n1 ; "C181" ; Cc1cn2nc(-c3ccc4c(=O)n(C5CCNCC5)ccc4c3)cc(CC(=O)O)c2n1 ; "531"]

To a mixture of 2-[6-[2-(1-tert-butoxycarbonyl-4-piperidyl)-1-oxo-6-isoquinolyl]-2-methyl-imidazo[1,2-b]pyridazin-8-yl]acetic acid (50.0 mg, 96.6 umol, 1 eq) in dioxane (2.00 mL) was added HCl/dioxane (2 M, 2.00 mL) in one portion at 20° C. The mixture was stirred at 20° C. for 2 hrs. The mixture was concentrated in reduced pressure at 45° C. The residue was purified by triturated with methyl tert-butyl ether (3.00 mL) to afford 2-[2-methyl-6-[1-oxo-2-(4-piperidyl)-6-isoquinolyl]imidazo[1,2-b]pyridazin-8-yl]acetic acid (20.3 mg, 50%) as a solid. LCMS: (ESI, m/z): 418.1 [M+H]⁺. ¹H NMR (400 MHz, MeOD-d₄) δ ppm 8.54 (d, J=8.80 Hz, 1H) 8.45 (s, 2H) 8.40 (s, 1H) 8.29 (dd, J=8.56, 1.71 Hz, 1H) 7.54 (d, J=7.34 Hz, 1H) 6.95 (d, J=7.82 Hz, 1H) 5.13-5.23 (m, 1H) 4.27-4.32 (m, 2H) 3.61-3.68 (m, 2H) 3.26-3.31 (m, 2H) 2.68 (s, 3H) 2.38 (qd, J=13.04, 3.91 Hz, 2H) 2.20 (br d, J=13.20 Hz, 2H)

### Example 173: Synthesis of Compound 534

### Synthesis of Intermediate C182

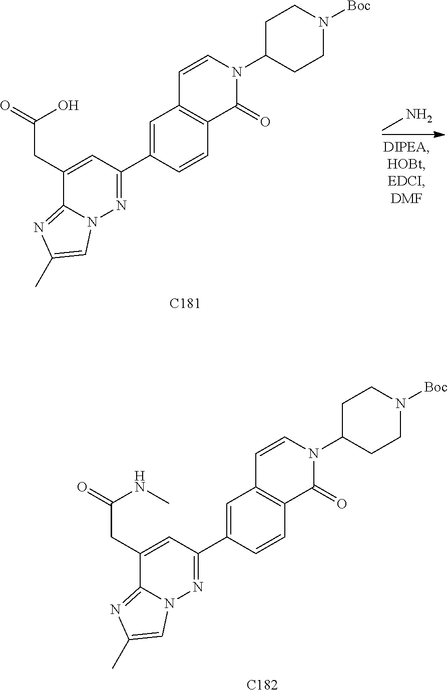

To a mixture of 2-[6-[2-(1-tert-butoxycarbonyl-4-piperidyl)-1-oxo-6-isoquinolyl]-2-methyl-imidazo[1,2-b]pyridazin-8-yl]acetic acid (50.0 mg, 96.6 umol, 1 eq) and N,N-diisopropylethylamine (24.9 mg, 193 umol, 33.6 uL, 2 eq), N1-((ethylimino)methylene)-N3,N3-dimethylpropane-1,3-diamine (30.0 mg, 193 umol, 34.2 uL, 2 eq) and 1H-benzo[d][1,2,3]triazol-1-ol (26.1 mg, 193 umol, 2 eq) in N,N-dimethylformamide (7.50 mL) was added methylamine; hydrochloride (6.12 mg, 193 umol, 2 eq) at 20° C. under N₂. The mixture was stirred at 20° C. for 16 hrs. The reaction mixture was quenched with water (15 mL) at 20° C. and extracted with ethyl acetate (3×6 mL). The combined organic layer was dried over Na₂SO₄, filtered and the filtrate was concentrated under reduced pressure to give tert-butyl 4-(6-(2-methyl-8-(2-(methylamino)-2-oxoethyl) imidazo[1,2-b]pyridazin-6-yl)-1-oxoisoquinolin-2(1H)-yl) piperidine-1-carboxylate (50.0 mg, 49%) as an oil. The crude product was used in the next step directly without further purification. LCMS: (ESI, m/z): 531.3 [M+H]⁺

### Synthesis of Compound 534

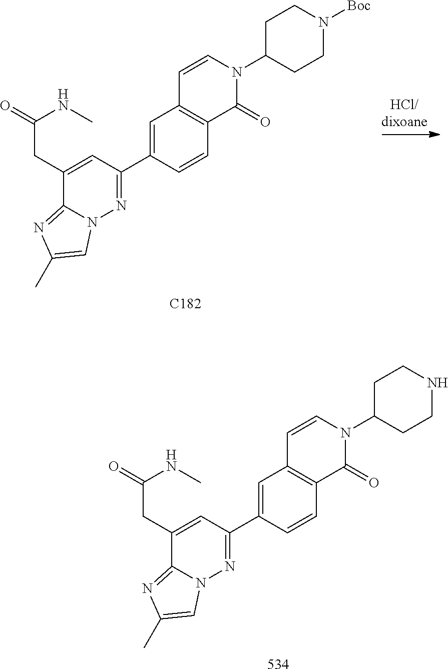 [SMILES: CNC(=O)Cc1cc(-c2ccc3c(=O)n(C4CCN(C(=O)OC(C)(C)C)CC4)ccc3c2)nn2cc(C)nc12 ; "C182" ; CNC(=O)Cc1cc(-c2ccc3c(=O)n(C4CCNCC4)ccc3c2)nn2cc(C)nc12 ; "534"]

To a mixture of tert-butyl 4-(6-(2-methyl-8-(2-(methylamino)-2-oxoethyl) imidazo[1,2-b]pyridazin-6-yl)-1-oxoisoquinolin-2(1H)-yl) piperidine-1-carboxylate (0.0 mg, 96.6 umol, 1 eq) in dioxane (100 uL) was added HCl/dioxane. The mixture was stirred at 25° C. for 1 hr. The reaction mixture was filtered and the filter cake was purified by prep-HPLC (Condition 14, Gradient 1) to afford N-methyl-2-[2-methyl-6-[1-oxo-2-(4-piperidyl)-6-isoquinolyl]imidazo[1,2-b]pyridazin-8-yl]acetamide (12.5 mg, 24%) as a solid. LCMS: (ESI, m/z): 431.1 [M+H]^{+ 1}H NMR (400 MHz, CDCl₃) δ ppm 8.56 (d, J=8.34 Hz, 1H) 8.04-8.12 (m, 2H) 7.83 (s, 1H) 7.58 (s, 1H) 7.44 (br d, J=4.29 Hz, 1H) 6.65 (d, J=7.39 Hz, 1H) 5.08-5.26 (m, 1H) 4.00 (s, 2H) 3.27 (br d, J=12.28 Hz, 2H) 2.85-2.94 (m, 2H) 2.79 (d, J=4.77 Hz, 3H) 2.55 (s, 3H) 1.92-1.99 (m, 2H) 1.76-1.85 (m, 2H).

### Example 174: Synthesis of Compound 532

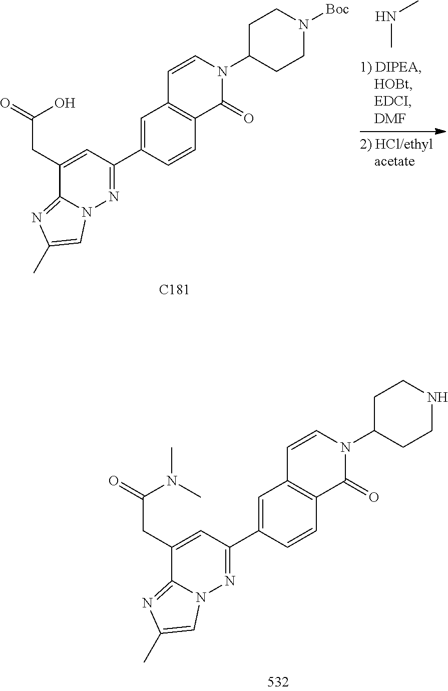

A mixture of 2-[6-[2-(1-tert-butoxycarbonyl-4-piperidyl)-1-oxo-6-isoquinolyl]-2-methyl-imidazo[1,2-b]pyridazin-8-yl]acetic acid (100 mg, 193 umol, 1 eq) and 3-(ethyliminomethylideneamino)propyl-dimethylazanium chloride (74.0 mg, 386 umol, 2 eq), Me₂NH (1 M, 386 μL, 2 eq) in DMF (2.00 mL) was added 1-hydroxybenzotriazole (52.2 mg, 386 umol, 2 eq) and N-ethyl-N-propan-2-ylpropan-2-amine (49.9 mg, 386 umol, 67.3 uL, 2 eq). The mixture was stirred at 25° C. for 12 hrs under N₂ atmosphere. Then HCl/ethyl acetate (4 M, 1.05 mL) was added to the above solution. The mixture was stirred at 25° C. for 1 hr under N₂ atmosphere. TLC (petroleum ether/ethyl acetate=3/1, Rf=0.01) showed the starting material was consumed and a new major spot was generated. The mixture was concentrated to give a residue, which was purified by prep-HPLC (Condition 13, Gradient 2) to afford N,N-dimethyl-2-[2-methyl-6-[1-oxo-2-(4-piperidyl)-6-isoquinolyl]imidazo[1,2-b]pyridazin-8-yl]acetamide (20.1 mg, 23%) as a solid. LCMS: (ESI, m/z): 445.1 [M+H]^{+ 1}H NMR (MeOD-d₄, 400 MHz) δ 8.53 (d, 1H, J=8.5 Hz), 8.4-8.5 (m, 3H), 8.28 (d, 1H, J=8.5 Hz), 7.55 (d, 1H, J=7.5 Hz), 6.94 (d, 1H, J=7.4 Hz), 5.19 (ddd, 1H, J=3.8, 8.6, 12.1 Hz), 4.44 (s, 2H), 3.65 (br d, 2H, J=12.9 Hz), 3.3-3.4 (m, 1H), 3.31 (s, 3H), 3.28 (br s, 1H), 3.06 (s, 3H), 2.68 (s, 3H), 2.39 (dq, 2H, J=3.6, 13.0 Hz), 2.19 (br d, 2H, J=13.1 Hz)

### Example 175: Synthesis of Compound 535

### Synthesis of Intermediate C183

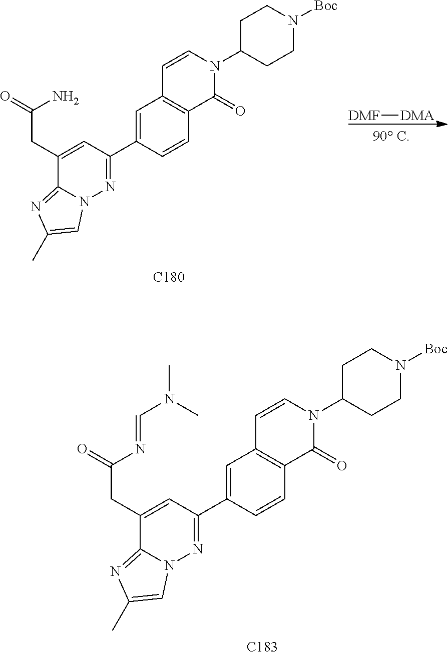

To a solution of tert-butyl 4-[6-[8-(2-amino-2-oxo-ethyl)-2-methyl-imidazo[1,2-b]pyridazin-6-yl]-1-oxo-2-isoquinolyl]piperidine-1-carboxylate (100 mg, 193 umol, 1 eq) in DMF-DMA (1.79 g, 15.0 mmol, 2 mL) was stirred at 90° C. for 2 hrs. The reaction mixture was concentrated under reduced pressure. The crude product tert-butyl 4-[6-[8-[2-[(E)-dimethylaminomethyleneamino]-2-oxo-ethyl]-2-methyl-imidazo[1,2-b]pyridazin-6-yl]-1-oxo-2-isoquinolyl]piperidine-1-carboxylate (110 mg) was used directly into the next step without further purification. LCMS: (ESI, m/z): 627.4 [M+H+(DMF−DMA)−2 MeOH]⁺.

### Synthesis of Intermediate C184

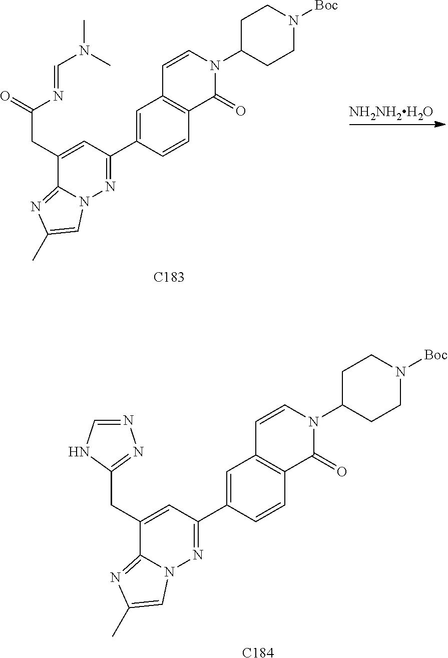 [SMILES: Cc1cn2nc(-c3ccc4c(=O)n(C5CCN(C(=O)OC(C)(C)C)CC5)ccc4c3)cc(CC(=O)/N=C/N(C)C)c2n1 ; "NH2NH2·H2O" ; "C183" ; Cc1cn2nc(-c3ccc4c(=O)n(C5CCN(C(=O)OC(C)(C)C)CC5)ccc4c3)cc(Cc3nnc[nH]3)c2n1 ; "C184"]

To a solution of tert-butyl 4-[6-[8-[2-[(E)-dimethylaminomethyleneamino]-2-oxo-ethyl]-2-methyl-imidazo[1,2-b]pyridazin-6-yl]-1-oxo-2-isoquinolyl]piperidine-1-carboxylate (110 mg, 193 umol, 1 eq) in CH₃COOH (1.00 mL) and EtOH (500 uL) was added NH₂NH₂·H₂O (515 mg, 10.2 mmol, 500 uL) at −10° C. and the mixture was stirred at 25° C. for 2 hrs. Then the reaction was warmed up to 40° C. and stirred for 8 hrs. The reaction mixture was concentrated under reduced pressure to remove solvent. The residue was purified by prep-TLC (SiO₂, DCM:MeOH=10:1, Rf=0.25 in UV) to give tert-butyl 4-[6-[2-methyl-8-(4H-1,2,4-triazol-3-ylmethyl)imidazo[1,2-b]pyridazin-6-yl]-1-oxo-2-isoquinolyl]piperidine-carboxylate (30.0 mg, 55.4 umol, 29%) as a solid. LCMS: (ESI, m/z): 541.3 [M+H]⁺. ¹H NMR (400 MHz, CDCl₃) δ ppm 8.56 (d, J=9.01 Hz, 1H) 8.01-8.09 (m, 2H) 7.93 (s, 1H) 7.85 (s, 1H) 7.55 (s, 1H) 7.17 (d, J=7.63 Hz, 1H) 6.65 (d, J=7.50 Hz, 1H) 5.21 (tt, J=12.24, 3.71 Hz, 1H) 4.20-4.48 (m, 2H) 4.60 (s, 2H) 2.82-3.06 (m, 2H) 2.60 (s, 3H) 1.94(brd, J=11.26 Hz, 2H) 1.76-1.84 (m, 2H) 1.48-1.54 (m, 9H)

### Synthesis of Compound 535

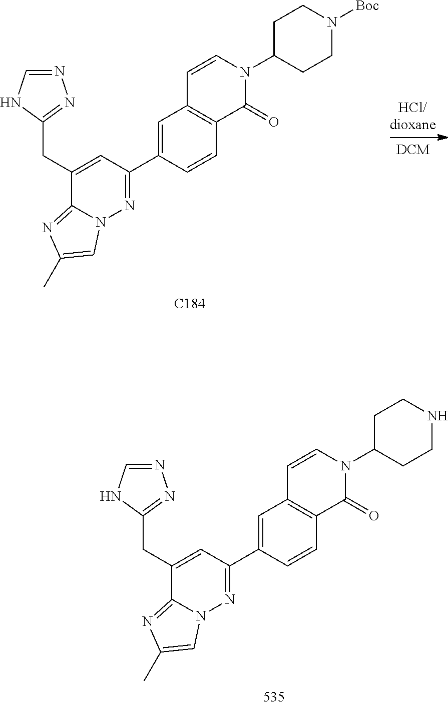 [SMILES: Cc1cn2nc(-c3ccc4c(=O)n(C5CCN(C(=O)OC(C)(C)C)CC5)ccc4c3)cc(Cc3nnc[nH]3)c2n1 ; "C184" ; Cc1cn2nc(-c3ccc4c(=O)n(C5CCNCC5)ccc4c3)cc(Cc3nnc[nH]3)c2n1 ; "535"]

To a solution of tert-butyl 4-[6-[2-methyl-8-(4H-1,2,4-triazol-3-ylmethyl)imidazo[1,2-b]pyridazin-6-yl]-1-oxo-2-isoquinolyl]piperidine-1-carboxylate (30.0 mg, 55.4 umol, 1 eq) in DCM (1.00 mL) was added HCl/dioxane (4 M, 2.00 mL). The mixture was stirred at 25° C. for 1 hr. The reaction mixture was concentrated under reduced pressure to give a residue. The crude product was triturated with DCM (3.00 mL) at 25° C. for 60 min to give. 6-[2-methyl-8-(4H-1,2,4-triazol-3-ylmethyl) imidazo[1,2-b]pyridazin-6-yl]-2-(4-piperidyl) isoquinolin-1-one (8.70 mg, 32%) as a solid. LCMS: (ESI, m/z): 441.1 [M+H]⁺. ¹HNMR (400 MHz, CD₃OD) δ ppm=8.91 (br s, 1H) 8.57-8.41 (m, 4H) 8.27 (dd, J=1.6, 8.5 Hz, 1H) 7.52 (d, J=7.5 Hz, 1H), 6.92 (d, J=7.6 Hz, 1H) 5.15 (ddd, J=4.0, 8.4, 12.2 Hz, 1H) 4.80-4.73 (m, 2H) 3.62 (br d, J=13.0 Hz, 2H) 3.30-3.26 (m, 2H) 2.68 (s, 3H) 2.36 (dq, J=3.8, 12.9 Hz, 2H) 2.18 (br d, J=12.8 Hz, 2H).

### Example 176: Synthesis of Compound 533

### Synthesis of Intermediate C185

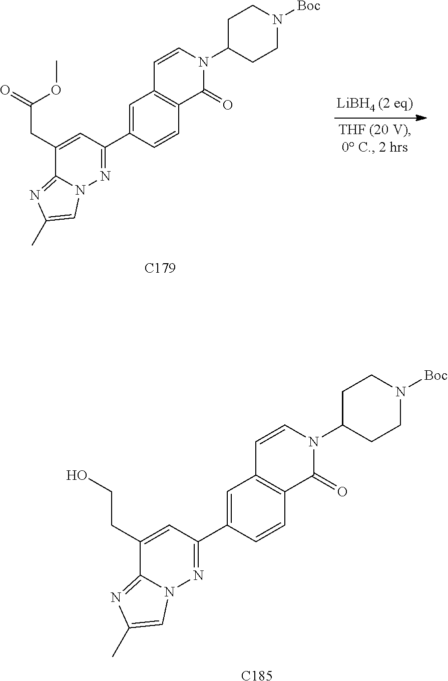 [SMILES: COC(=O)Cc1cc(-c2ccc3c(=O)n(C4CCN(C(=O)OC(C)(C)C)CC4)ccc3c2)nn2cc(C)nc12 ; "C179" ; Cc1cn2nc(-c3ccc4c(=O)n(C5CCN(C(=O)OC(C)(C)C)CC5)ccc4c3)cc(CCO)c2n1 ; "C185"]

To a solution of tert-butyl 4-[6-[8-(2-methoxy-2-oxo-ethyl)-2-methyl-imidazo[1,2-b]pyridazin-6-yl]-1-oxo-2-isoquinolyl]piperidine-1-carboxylate (100 mg, 188 umol, 1 eq) in tetrahydrofuran (2.00 mL) was added LiBH₄ (8.20 mg, 376 umol, 2 eq) at 0° C. under N₂. The reaction mixture was stirred at 0° C. for 2 hrs. The mixture was poured into ice-water (20.0 mL) and the resulting mixture was stirred for 0.5 hr. The mixture was extracted with ethyl acetate (3×20 mL). The combined organic phase was washed with brine (3×20 mL), dried with anhydrous Na₂SO₄, filtered and concentrated to give a residue, which was purified by silica gel column chromatography (ethyl acetate) to give tert-butyl 4-[6-[8-(2-hydroxyethyl)-2-methyl-imidazo[1,2-b]pyridazin-6-yl]-1-oxo-2-isoquinolyl]piperidine-1-carboxylate (50.0 mg, 40%) as a solid. LCMS: (ESI, m/z): 504.3 [M+H]^{+ 1}H NMR (400 MHz, MeOD-d₄) δ ppm 8.44 (d, J=8.56 Hz, 1H) 8.27 (d, J=1.47 Hz, 1H) 8.18 (dd, J=8.50, 1.77 Hz, 1H) 7.96 (d, J=0.61 Hz, 1H) 7.71 (s, 1H) 7.51 (d, J=7.58 Hz, 1H) 6.84 (d, J=7.58 Hz, 1H) 5.05-5.15 (m, 1H) 4.32 (br d, J=12.96 Hz, 2H) 4.04 (t, J=6.17 Hz, 2H) 3.27 (t, J=6.17 Hz, 2H) 2.88-3.09 (m, 2H) 1.86-1.96 (m, 4H) 1.50 (s, 9H)

### Synthesis of Compound 533

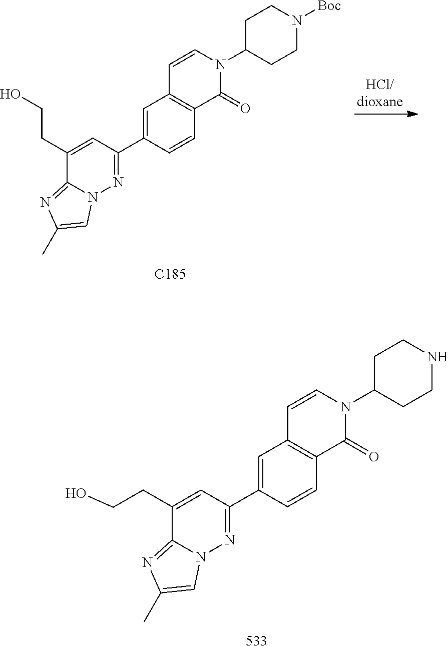 [SMILES: Cc1cn2nc(-c3ccc4c(=O)n(C5CCN(C(=O)OC(C)(C)C)CC5)ccc4c3)cc(CCO)c2n1 ; "C185" ; Cc1cn2nc(-c3ccc4c(=O)n(C5CCNCC5)ccc4c3)cc(CCO)c2n1 ; "533"]

To a solution of tert-butyl 4-[6-[8-(2-hydroxyethyl)-2-methyl-imidazo[1,2-b]pyridazin-6-yl]-1-oxo-2-isoquinolyl]piperidine-1-carboxylate (50.0 mg, 99.3 umol, 1 eq) in dioxane (1.00 mL) was added HCl/dioxane (4 M, 1.00 mL) at 25° C. under N₂. The reaction mixture was stirred at 25° C. for 1 hr. The mixture was concentrated to give a residue. The crude product was purified by reversed-phase HPLC (Condition 13, Gradient 2) to afford 6-[8-(2-hydroxyethyl)-2-methyl-imidazo[1,2-b]pyridazin-6-yl]-2-(4-piperidyl)isoquinolin-1-one (25.9 mg, 63%) as a solid. LCMS: (ESI, m/z): 404.1 [M+H]^{+ 1}H NMR (400 MHz, MeOD-d₄) δ ppm 8.53 (d, J=8.44 Hz, 1H) 8.46 (s, 1H) 8.39 (d, J=7.58 Hz, 2H) 8.29 (br d, J=8.80 Hz, 1H) 7.53 (d, J=7.58 Hz, 1H) 6.94 (d, J=7.58 Hz, 1H) 5.11-5.23 (m, 1H) 4.08 (t, J=5.75 Hz, 2H) 3.63 (br d, J=12.72 Hz, 2H) 3.33 (br d, J=5.87 Hz, 2H) 3.27 (br s, 2H) 2.68 (s, 3H) 2.36 (br dd, J=12.96, 3.42 Hz, 2H) 2.18 (br d, J=13.33 Hz, 2H)

### Example 177: Synthesis of Compound 525

### Synthesis of Intermediate C186

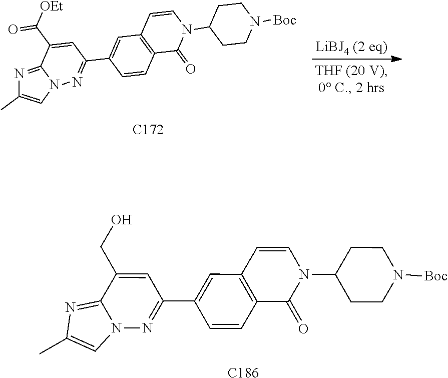 [SMILES: CCOC(=O)c1cc(-c2ccc3c(=O)n(C4CCN(C(=O)OC(C)(C)C)CC4)ccc3c2)nn2cc(C)nc12 ; "C172" ; Cc1cn2nc(-c3ccc4c(=O)n(C5CCN(C(=O)OC(C)(C)C)CC5)ccc4c3)cc(CO)c2n1 ; "C186"]

To a solution of ethyl 6-[2-(1-tert-butoxycarbonyl-4-piperidyl)-1-oxo-6-isoquinolyl]-2-methyl-imidazo[1,2-b]pyridazine-8-carboxylate (200 mg, 376 umol, 1 eq) in tetrahydrofuran (4.00 mL) was added LiBH₄ (16.4 mg, 752 umol, 2 eq) at 0° C. under N₂. The reaction mixture was stirred at 0° C. for 2 hrs. The mixture was poured into ice-water (20.0 mL) and stirred for 0.5 hr. The aqueous phase was extracted with ethyl acetate (3×20.0 mL). The combined organic phase was washed with brine (3×20.0 mL), dried with anhydrous Na₂SO₄, filtered and concentrated to give a residue, which was purified by silica gel column chromatography (ethyl acetate) to give tert-butyl 4-[6-[8-(hydroxymethyl)-2-methyl-imidazo[1,2-b]pyridazin-6-yl]-1-oxo-2-isoquinolyl]piperidine-1-carboxylate (75.0 mg, 36%) as a solid. LCMS: (ESI, m/z): 490.3 [M+H]^{+ 1}H NMR (400 MHz, DMSO-d₆) δ ppm 8.30-8.45 (m, 2H) 8.13-8.18 (m, 2H) 7.80 (s, 1H) 7.65 (d, J=7.58 Hz, 1H) 6.85 (d, J=7.58 Hz, 1H) 5.76 (t, J=5.62 Hz, 1H) 4.99-5.05 (m, 1H) 4.97 (d, J=5.38 Hz, 2H) 4.08-4.23 (m, 2H) 2.83-3.03 (m, 2H) 2.42 (s, 3H) 1.70-1.90 (m, 4H) 1.44 (s, 9H)

### Synthesis of Compound 525

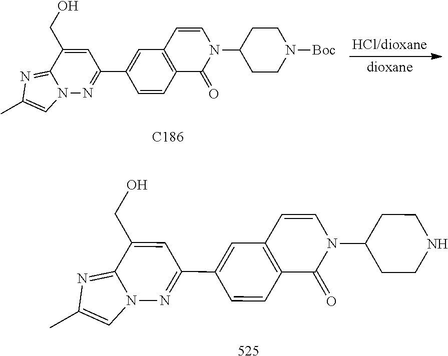 [SMILES: Cc1cn2nc(-c3ccc4c(=O)n(C5CCN(C(=O)OC(C)(C)C)CC5)ccc4c3)cc(CO)c2n1 ; "C186" ; Cc1cn2nc(-c3ccc4c(=O)n(C5CCNCC5)ccc4c3)cc(CO)c2n1 ; "525"]

To a solution of tert-butyl 4-[6-[8-(hydroxymethyl)-2-methyl-imidazo[1,2-b]pyridazin-6-yl]-1-oxo-2-isoquinolyl]piperidine-1-carboxylate (50.0 mg, 102 umol, 1 eq) in dioxane (1.00 mL) was added HCl/dioxane (4 M, 1.00 mL) at 25° C. under N₂. The reaction mixture was stirred at 25° C. for 1 hr. The reaction mixture was concentrated under reduced pressure to give the crude product. The crude product was triturated with methyl tert-butyl ether (1.00 mL). The solid was filtered and concentrated under reduced pressure to give the 6-[8-(hydroxymethyl)-2-methyl-imidazo[1,2-b]pyridazin-6-yl]-2-(4-piperidyl) isoquinolin-1-one (28.8 mg, 70%). LCMS: (ESI, m/z): 390.1 [M+H]^{+ 1}H NMR (400 MHz, MeOD-d₄) δ ppm 8.54 (d, J=8.68 Hz, 1H) 8.43 (d, J=1.35 Hz, 1H) 8.38 (d, J=6.85 Hz, 2H) 8.28 (dd, J=8.56, 1.59 Hz, 1H) 7.50 (d, J=7.58 Hz, 1H) 6.94 (d, J=7.58 Hz, 1H) 5.10-5.16 (m, 3H) 3.63 (br d, J=12.96 Hz, 2H) 3.26 (br d, J=2.32 Hz, 2H) 2.66 (s, 3H) 2.34 (br dd, J=13.02, 3.48 Hz, 2H) 2.19 (br d, J=13.33 Hz, 2H)

### Example 178: Synthesis of Compound 519

### Synthesis of Intermediate C188

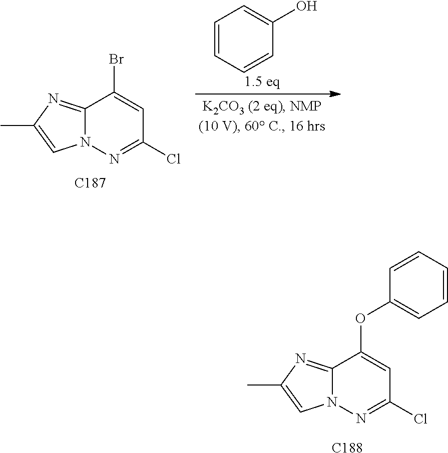 [SMILES: Cc1cn2nc(Cl)cc(Br)c2n1 ; Oc1ccccc1 ; "C187" ; Cc1cn2nc(Cl)cc(Oc3ccccc3)c2n1 ; "C188"]

To a solution of phenol (286 mg, 3.04 mmol, 267 μL, 1.5 eq) and 8-bromo-6-chloro-2-methyl-imidazo[1,2-b]pyridazine (500 mg, 2.03 mmol, 1 eq) in N-methyl-2-pyrrolidone (5.00 mL) was added K₂CO₃ (560 mg, 4.06 mmol, 2 eq) at 20° C. The reaction mixture was heated to 60° C. and stirred for 16 hrs. After cooling to 20° C., the reaction mixture was quenched by ice-water (20.0 mL) slowly and the resulting mixture was extracted with ethyl acetate (3×5 mL). The combined organic phase was washed with brine (15.0 mL), dried over anhydrous Na₂SO₄, filtered and concentrated in vacuum. The residue was purified by silica gel column chromatography (petroleum ether/ethyl acetate=1:1) to afford give 6-chloro-2-methyl-8-phenoxy-imidazo[1,2-b]pyridazine (400 mg, 76%) as a solid. LCMS: (ESI, m/z): 260.0 [M+H]⁺. ¹H NMR (400 MHz, CDCl₃) δ ppm 7.69 (s, 1H) 7.48-7.54 (m, 2H) 7.34-7.39 (m, 1H) 7.23-7.26 (m, 2H) 6.10 (s, 1H) 2.53 (d, J=0.98 Hz, 3H)

### Synthesis of Intermediate C189

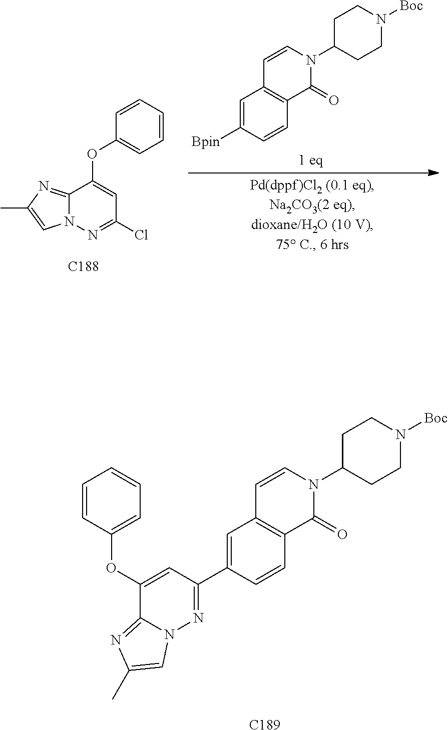

To a mixture of tert-butyl 4-[1-oxo-6-(4,4,5,5-tetramethyl-1,3,2-dioxaborolan-2-yl)-2-isoquinolyl]piperidine-1-carboxylate (200 mg, 440 umol, 1 eq) and 6-chloro-2-methyl-8-phenoxy-imidazo[1,2-b]pyridazine (114 mg, 440 umol, 1 eq) in dioxane (1.50 mL) and water (500 uL) was added [1,1-bis(diphenylphosphino)ferrocene]dichloropalladium(II) (32.2 mg, 44.0 umol, 0.1 eq) and Na₂CO₃ (93.3 mg, 880 umol, 2 eq) at 20° C. under N₂ protection. The mixture was stirred at 75° C. for 6 hrs. After cooling to 20° C., the reaction mixture was concentrated to give a residue, which was purified by silica gel chromatography (petroleum ether/ethyl acetate=1/1) to afford tert-butyl 4-[6-(2-methyl-8-phenoxy-imidazo[1,2-b]pyridazin-6-yl)-1-oxo-2-isoquinolyl]piperidine-1-carboxylate (100 mg, 41%) as a solid. LCMS: (ESI, m/z): 552.3 [M+H]⁺. ¹H NMR (400 MHz, CDCl₃) δ ppm 8.48 (d, J=8.31 Hz, 1H) 7.89 (d, J=1.47 Hz, 1H) 7.82-7.86 (m, 2H) 7.49-7.57 (m, 2H) 7.35-7.40 (m, 1H) 7.29-7.33 (m, 2H) 7.13 (d, J=7.34 Hz, 1H) 6.56-6.61 (m, 2H) 5.18 (s, 1H) 4.32 (br d, J=1.47 Hz, 2H) 2.93 (br s, 2H) 2.59 (s, 3H) 1.91 (br d, J=12.23 Hz, 2H) 1.68-1.80 (m, 2H) 1.50 (s, 9H)

### Synthesis of Compound 519

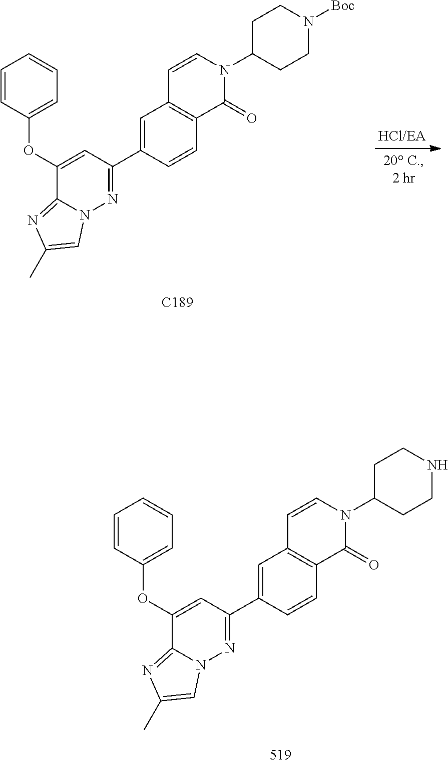 [SMILES: Cc1cn2nc(-c3ccc4c(=O)n(C5CCN(C(=O)OC(C)(C)C)CC5)ccc4c3)cc(Oc3ccccc3)c2n1 ; "C189" ; Cc1cn2nc(-c3ccc4c(=O)n(C5CCNCC5)ccc4c3)cc(Oc3ccccc3)c2n1 ; "519"]

To a mixture of tert-butyl 4-[6-(2-methyl-8-phenoxy-imidazo[1,2-b]pyridazin-6-yl)-1-oxo-2-isoquinolyl]piperidine-1-carboxylate (100 mg, 181 umol, 1 eq) in ethyl acetate (2.00 mL) was added HCl/ethyl acetate (4 M, 2.00 mL) at 20° C. The mixture was stirred at 20° C. for 2 hrs. The mixture was concentrated to give a residue, which was purified by prep-HPLC (Condition 12, Gradient 2) to afford 6-(2-methyl-8-phenoxy-imidazo[1,2-b]pyridazin-6-yl)-2-(4-piperidyl)isoquinolin-1-one (26.8 mg, 30%) as a solid. LCMS: (ESI, m/z): 452.1 [M+H]⁺. ¹H NMR (400 MHz, MeOD-d₄) δ ppm 8.38 (d, J=8.80 Hz, 1H) 8.02 (br d, J=6.85 Hz, 2H) 7.95 (br d, J=8.31 Hz, 1H) 7.55-7.62 (m, 2H) 7.35-7.49 (m, 4H) 6.76-6.81 (m, 2H) 4.98-5.09 (m, 1H) 3.22 (br d, J=13.20 Hz, 2H) 2.77-2.87 (m, 2H) 2.52 (s, 3H) 1.84-1.92 (m, 4H)

### Example 179: Synthesis of Compound 526

### Synthesis of Intermediate C190

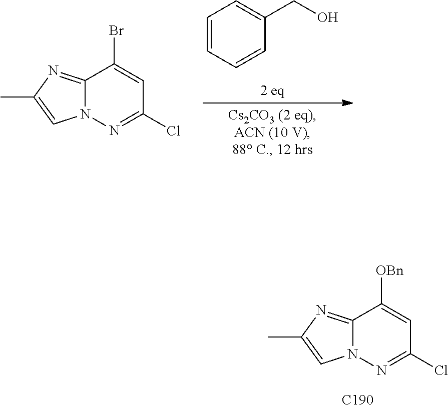 [SMILES: Cc1cn2nc(Cl)cc(Br)c2n1 ; OCc1ccccc1 ; Cc1cn2nc(Cl)cc(OCc3ccccc3)c2n1 ; "C190"]

To a solution of 8-bromo-6-chloro-2-methyl-imidazo[1,2-b]pyridazine (500 mg, 2.03 mmol, 1 eq) and phenylmethanol (438 mg, 4.06 mmol, 421 μL, 2 eq) in acetonitrile (5.00 mL) was added Cs₂CO₃ (1.32 g, 4.06 mmol, 2 eq). The reaction mixture was heated to 88° C. and stirred for 12 hrs. After cooling to 20° C., the reaction mixture was quenched by ice water (10.0 mL) slowly and the resulting mixture was extracted with ethyl acetate (3×10 mL). The combined organic phase was washed with brine (15 mL), dried over anhydrous Na₂SO₄, filtered and concentrated in vacuum. The residue was purified by prep-HPLC (Condition 12, Gradient 3) to afford 8-benzyloxy-6-chloro-2-methyl-imidazo[1,2-b]pyridazine (300 mg, 54%) as a solid. LCMS: (ESI, m/z): 274.1 [M+H]⁺. ¹H NMR (400 MHz, CDCl₃) δ ppm 7.62 (s, 1H) 7.47-7.50 (m, 2H) 7.37-7.44 (m, 3H) 6.40 (s, 1H) 5.39 (s, 2H) 2.48 (s, 3H)

### Synthesis of Intermediate C191

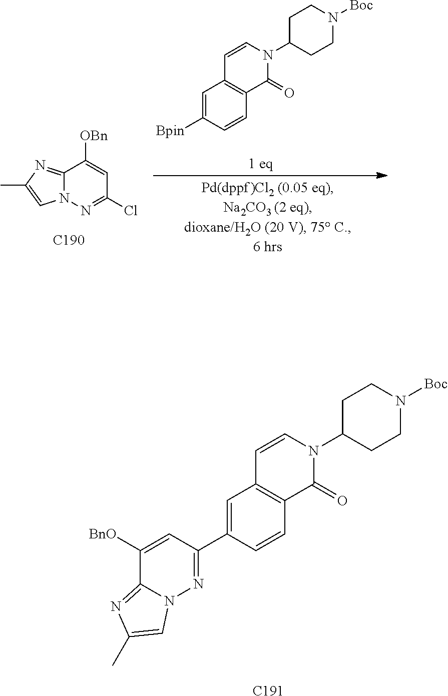

To a mixture of tert-butyl 4-[1-oxo-6-(4,4,5,5-tetramethyl-1,3,2-dioxaborolan-2-yl)-2-isoquinolyl]piperidine-1-carboxylate (200 mg, 440 umol, 1 eq) and 8-benzyloxy-6-chloro-2-methyl-imidazo[1,2-b]pyridazine (120 mg, 440 umol, 1 eq) in dioxane (3.00 mL) and water (1.00 mL) was added [1,1-bis(diphenylphosphino)ferrocene]dichloropalladium(II) (16.1 mg, 22.0 umol, 0.05 eq) and Na₂CO₃ (93.3 mg, 880 umol, 2 eq) at 20° C. under N₂ protection. The mixture was stirred at 75° C. for 6 hrs. After cooling to 20° C., the reaction mixture was concentrated to give a residue, which was purified by silica gel chromatography (petroleum ether/ethyl acetate=1/1) to afford tert-butyl 4-[6-(8-benzyloxy-2-methyl-imidazo[1,2-b]pyridazin-6-yl)-1-oxo-2-isoquinolyl]piperidine-1-carboxylate (100 mg, 40%) as a solid. LCMS: (ESI, m/z): 566.2 [M+H]⁺. ¹H NMR (400 MHz, MeOD-d₄) δ ppm 8.45 (d, J=8.50 Hz, 1H) 8.27 (d, J=1.50 Hz, 1H) 8.16 (dd, J=8.50, 1.50 Hz, 1H) 7.90 (s, 1H) 7.62 (d, J=7.00 Hz, 2H) 7.52 (d, J=7.00 Hz, 1H) 7.36-7.47 (m, 3H) 7.32 (s, 1H) 6.85 (d, J=7.50 Hz, 1H) 5.54 (s, 2H) 5.05-5.19 (m, 1H) 4.58 (s, 2H) 4.32 (br d, J=12.51 Hz, 2H) 2.45 (s, 3H) 1.87-1.96 (m, 4H) 1.50 (s, 9H)

### Synthesis of Compound 526

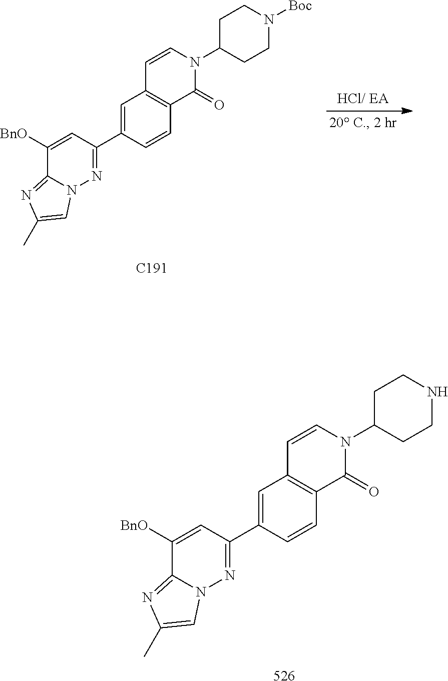 [SMILES: Cc1cn2nc(-c3ccc4c(=O)n(C5CCN(C(=O)OC(C)(C)C)CC5)ccc4c3)cc(OCc3ccccc3)c2n1 ; "C191" ; Cc1cn2nc(-c3ccc4c(=O)n(C5CCNCC5)ccc4c3)cc(OCc3ccccc3)c2n1 ; "526"]

To a suspension of tert-butyl 4-[6-(8-benzyloxy-2-methyl-imidazo[1,2-b]pyridazin-6-yl)-1-oxo-2-isoquinolyl]piperidine-1-carboxylate (100 mg, 176 umol, 1 eq) in ethyl acetate (2.00 mL) was added HCl/ethyl acetate (2 M, 2.00 mL) at 20° C. The mixture was stirred at 20° C. for 2 hrs. The mixture was concentrated under reduced pressure at 50° C. The crude product was triturated with methyl tert-butyl ether (4.00 mL). The solid was filtered and the filter cake was dried to give 6-(8-benzyloxy-2-methyl-imidazo[1,2-b]pyridazin-6-yl)-2-(4-piperidyl) isoquinolin-1-one (62.3 mg, 76%) as a solid. LCMS: (ESI, m/z): 466.1 [M+H]⁺. ¹H NMR (400 MHz, MeOD-d₄) δ ppm 8.53 (d, J=8.80 Hz, 1H) 8.46 (s, 1H) 8.25-8.31 (m, 2H) 7.99 (d, J=1.47 Hz, 1H) 7.65 (br d, J=6.85 Hz, 2H) 7.44-7.54 (m, 4H) 6.94 (d, J=7.82 Hz, 1H) 5.68 (s, 2H) 5.11-5.21 (m, 1H) 3.63 (br d, J=12.72 Hz, 2H) 3.27 (br s, 2H) 2.59 (s, 3H) 2.29-2.42 (m, 2H) 2.18 (br d, J=12.72 Hz, 2H)

### Example 180: Synthesis of Compound 514

### Synthesis of Intermediate C192

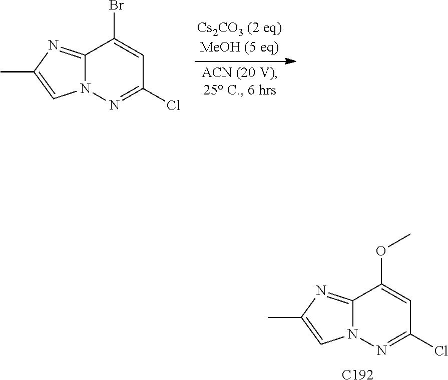

To a solution of 8-bromo-6-chloro-2-methyl-imidazo[1,2-b]pyridazine (500 mg, 2.03 mmol, 1 eq) and methanol (316 mg, 9.88 mmol, 400 μL, 5 eq) in acetonitrile (10.0 mL) was added Cs₂CO₃ (1.32 g, 4.06 mmol, 2 eq) at 20° C. The reaction mixture was stirred at 25° C. for 6 hrs. LCMS showed the reaction was completed. The reaction was quenched with water (20.0 mL) and the resulting mixture was extracted with ethyl acetate (3×5 mL). The combined organic phase was washed with brine (15.0 mL), dried over anhydrous Na₂SO₄, filtered and concentrated in vacuum. The residue was purified by silica gel column chromatography (Petroleum ether/Ethyl acetate=3:1)) to afford 6-chloro-8-methoxy-2-methyl-imidazo[1,2-b]pyridazine (300 mg, 75%) as a solid. LCMS: (ESI, m/z): 198.1 [M+H]⁺. ¹H NMR (400 MHz, CDCl₃) δ ppm 7.61 (s, 1H) 6.38 (s, 1H) 4.09 (s, 3H) 2.47 (s, 3H)

### Synthesis of Intermediate C193

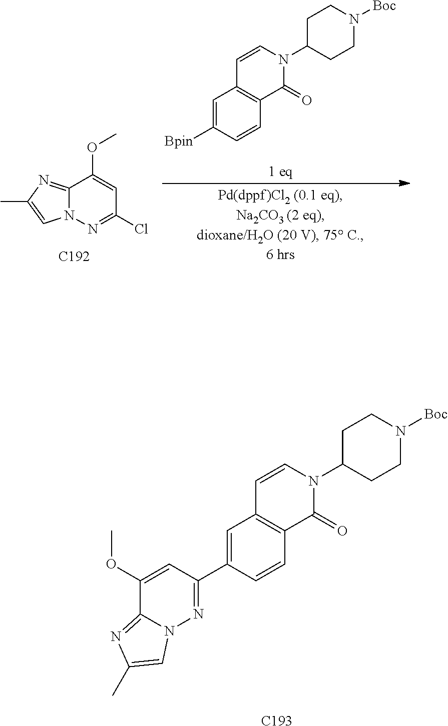

To a mixture of tert-butyl 4-[1-oxo-6-(4,4,5,5-tetramethyl-1,3,2-dioxaborolan-2-yl)-2-isoquinolyl]piperidine-1-carboxylate (200 mg, 440 umol, 1 eq) and 6-chloro-8-methoxy-2-methyl-imidazo[1,2-b]pyridazine (86.9 mg, 440 umol, 1 eq) in dioxane (3.00 mL) and water (1.00 mL) was added [1,1-bis(diphenylphosphino)ferrocene]dichloropalladium(II)(32.2 mg, 44.0 umol, 0.1 eq) and Na₂CO₃ (93.3 mg, 880 umol, 2 eq) under N₂ protection. The mixture was stirred at 75° C. for 6 hrs. After cooling to 20° C., the reaction mixture was concentrated to give a residue, which was purified by silica gel chromatography (petroleum ether/ethyl acetate=1/1) to afford tert-butyl 4-[6-(8-methoxy-2-methyl-imidazo[1,2-b]pyridazin-6-yl)-1-oxo-2-isoquinolyl]piperidine-1-carboxylate (100 mg, 46.4%) as a solid. LCMS: (ESI, m/z): 490.3 [M+H]⁺. ¹H NMR (400 MHz, DMSO-d₆) δ ppm 8.33-8.40 (m, 2H) 8.18 (dd, J=8.53, 1.51 Hz, 1H) 8.05 (s, 1H) 7.64 (d, J=7.53 Hz, 1H) 7.28 (s, 1H) 6.78 (d, J=7.53 Hz, 1H) 4.94-5.06 (m, 1H) 4.09-4.19 (m, 5H) 2.84-3.01 (m, 2H) 2.38 (s, 3H) 1.73-1.87 (m, 4H) 1.44 (s, 9H)

### Synthesis of Compound 514

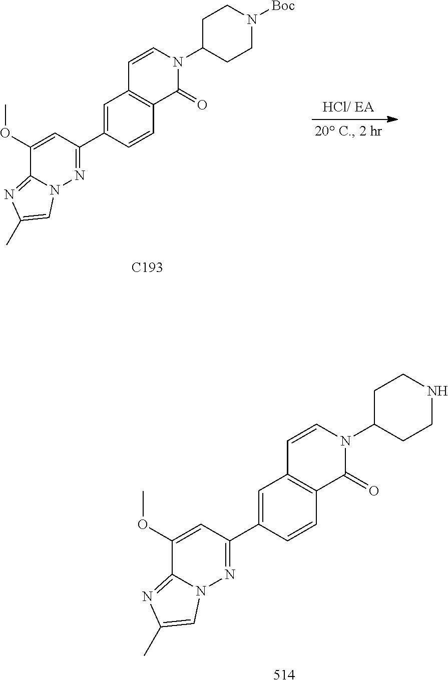 [SMILES: COc1cc(-c2ccc3c(=O)n(C4CCN(C(=O)OC(C)(C)C)CC4)ccc3c2)nn2cc(C)nc12 ; "C193" ; COc1cc(-c2ccc3c(=O)n(C4CCNCC4)ccc3c2)nn2cc(C)nc12 ; "514"]

To a mixture of tert-butyl 4-[6-(8-methoxy-2-methyl-imidazo[1,2-b]pyridazin-6-yl)-1-oxo-2-isoquinolyl]piperidine-1-carboxylate (100 mg, 204 umol, 1 eq) in ethyl acetate (2.00 mL) at 20° C. was added HCl/ethyl acetate (2 M, 2.00 mL). The mixture was stirred at 20° C. for 2 hrs. The mixture was concentrated under reduced pressure at 45° C. The crude product was triturated with methyl tert-butyl ether (10.0 mL). The solid was filtered and the filter cake was dried to give 6-(8-methoxy-2-methyl-imidazo[1,2-b]pyridazin-6-yl)-2-(4-piperidyl) isoquinolin-1-one; hydrochloride (80.7 mg, 93%) as a solid. LCMS: (ESI, m/z): 390.1 [M+H]⁺. ¹H NMR (400 MHz, MeOD-d₄) δ ppm 8.53 (d, J=8.80 Hz, 1H) 8.47 (d, J=1.47 Hz, 1H) 8.28-8.32 (m, 2H) 7.89 (s, 1H) 7.50 (d, J=7.83 Hz, 1H) 6.94 (d, J=7.82 Hz, 1H) 5.09-5.21 (m, 1H) 4.40 (s, 3H) 3.58-3.67 (m, 2H) 2.64 (d, J=0.98 Hz, 3H) 2.27-2.40 (m, 2H) 2.16-2.23 (m, 2H)

### Example 181: Synthesis of Compound 520

### Synthesis of Intermediate C194

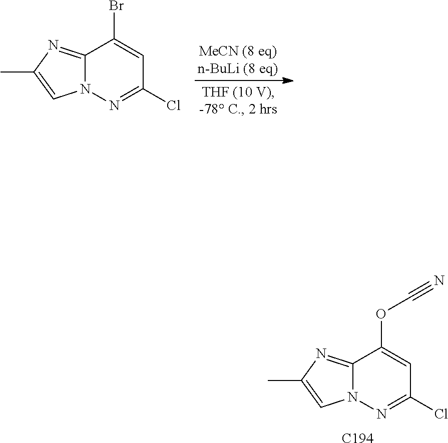

To a solution of n-butyllithium (2.5 M, 6.50 mL, 8 eq) in tetrahydrofuran (5.00 mL) was added acetonitrile (666 mg, 16.2 mmol, 855 μL, 8 eq) dropwise at −78° C. over a period of 30 min under N₂ protection. The reaction mixture was stirred at −78° C. for 30 min. A solution of 8-bromo-6-chloro-2-methyl-imidazo[1,2-b]pyridazine (500 mg, 2.03 mmol, 1 eq) in tetrahydrofuran (5.00 mL) was add to the above mixture at −78° C. The mixture reaction was stirred at −78° C. for another 1 hr. The reaction mixture was quenched by saturated NH₄Cl (10.0 mL) and the resulting mixture was extracted with ethyl acetate (3×10 mL). The combined organic phase was washed with brine (10.0 mL), dried over anhydrous Na₂SO₄, filtered and concentrated in vacuum. The residue was purified by silica gel column chromatography (Petroleum ether/Ethyl acetate=3/1) to give 2-(6-chloro-2-methyl-imidazo[1,2-b]pyridazin-8-yl) acetonitrile (100 mg, 24%) as a solid. LCMS: (ESI, m/z): 206.9 [M+H]⁺. ¹H NMR (400 MHz, CDCl₃) δ ppm 7.76 (s, 1H) 7.23 (s, 1H) 4.21 (s, 2H) 2.51 (s, 3H)

### Synthesis of Intermediate C195

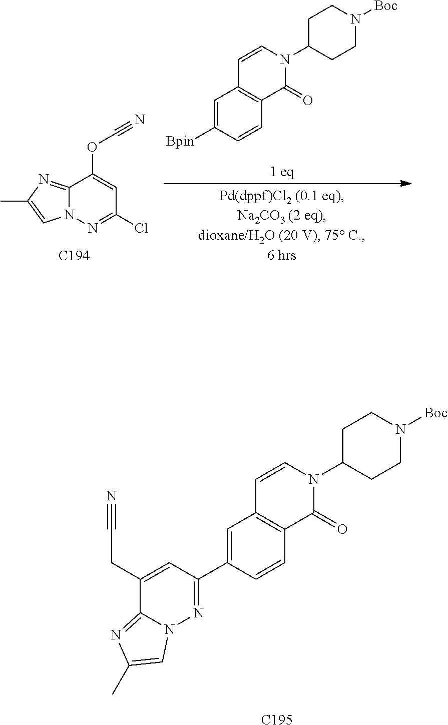

To a mixture of tert-butyl 4-[1-oxo-6-(4,4,5,5-tetramethyl-1,3,2-dioxaborolan-2-yl)-2-isoquinolyl]piperidine-1-carboxylate (EVAL-0116-4, 200 mg, 440 umol, 1 eq) and 2-(6-chloro-2-methyl-imidazo[1,2-b]pyridazin-8-yl)acetonitrile (90.9 mg, 440.17 umol, 1 eq) in dioxane (4.00 mL) and water (200 uL) was added [1,1 bis(diphenylphosphino)ferrocene]dichloropalladium(II) (16.1 mg, 22.0 umol, 0.05 eq) and Na₂CO₃ (93.3 mg, 880 umol, 2 eq) at 20° C. under N₂ protection. The mixture was stirred at 75° C. for 6 hrs. After cooling to 20° C., the reaction mixture was concentrated to give a residue, which was purified by silica gel chromatography (petroleum ether/ethyl acetate=1/1) to afford tert-butyl 4-[6-[8-(cyanomethyl)-2-methyl-imidazo[1,2-b]pyridazin-6-yl]-1-oxo-2-isoquinolyl]piperidine-1-carboxylate (100 mg, 46%) as a solid. LCMS: (ESI, m/z): 499.3 [M+H]⁺. ¹H NMR (400 MHz, DMSO-d₆) δ ppm 8.39 (d, J=8.80 Hz, 1H) 8.32 (d, J=1.96 Hz, 1H) 8.23 (s, 1H) 8.12 (dd, J=8.31, 1.47 Hz, 1H) 7.85 (s, 1H) 7.65 (d, J=7.34 Hz, 1H) 6.81 (d, J=7.34 Hz, 1H) 4.92-5.05 (m, 1H) 4.47 (s, 2H) 4.08-4.23 (m, 2H) 2.93 (br dd, J=4.65, 1.71 Hz, 2H) 2.45 (s, 3H) 1.74-1.86 (m, 4H) 1.44 (s, 9H)

### Synthesis of Compound 520

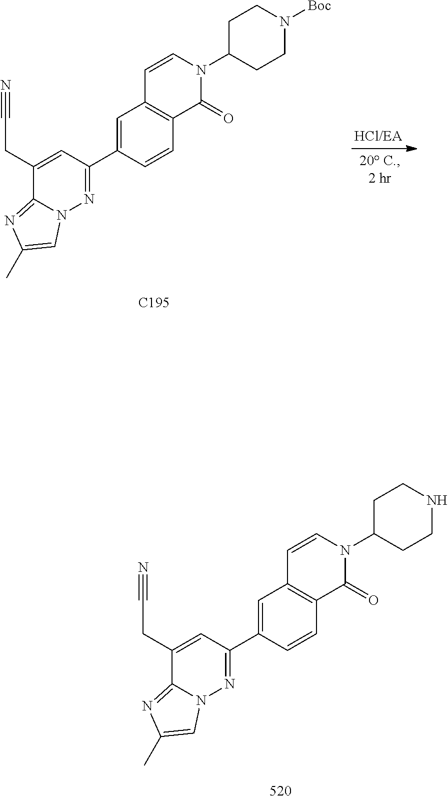 [SMILES: Cc1cn2nc(-c3ccc4c(=O)n(C5CCN(C(=O)OC(C)(C)C)CC5)ccc4c3)cc(CC#N)c2n1 ; "C195" ; Cc1cn2nc(-c3ccc4c(=O)n(C5CCNCC5)ccc4c3)cc(CC#N)c2n1 ; "520"]

To a mixture of tert-butyl 4-[6-[8-(cyanomethyl)-2-methyl-imidazo[1,2-b]pyridazin-6-yl]-1-oxo-2-isoquinolyl]piperidine-1-carboxylate (100 mg, 200 umol, 1.00 eq) in ethyl acetate (2.00 mL) was added HCl/ethyl acetate (4 M, 2.00 mL) at 20° C. under N₂ protection. The mixture was stirred at 20° C. for 2 hrs. The mixture was concentrated under reduced pressure at 45° C. The residue was purified by prep-HPLC (Condition 12, Gradient 4) to afford 2-[2-methyl-6-[1-oxo-2-(4-piperidyl)-6-isoquinolyl]imidazo[1,2-b]pyridazin-8-yl]acetonitrile (0.8 mg, 35%) as a solid. LCMS: (ESI, m/z): 399.1 [M+H]⁺. ¹H NMR (400 MHz, MeOD-d₄) δ ppm 8.47 (d, J=8.50 Hz, 1H) 8.30 (br s, 1H) 8.20 (br d, J=8.51 Hz, 1H) 8.07 (s, 1H) 7.87 (s, 1H) 7.53 (d, J=7.50 Hz, 1H) 6.89 (d, J=8.00 Hz, 1H) 5.08 (br t, J=7.50 Hz, 1H) 4.83-4.85 (m, 2H) 3.25 (br d, J=12.01 Hz, 2H) 2.81-2.89 (m, 2H) 2.53 (s, 3H) 1.88-1.96 (m, 4H)

### Example 182: Synthesis of Compound 536

### Synthesis of Intermediate C196

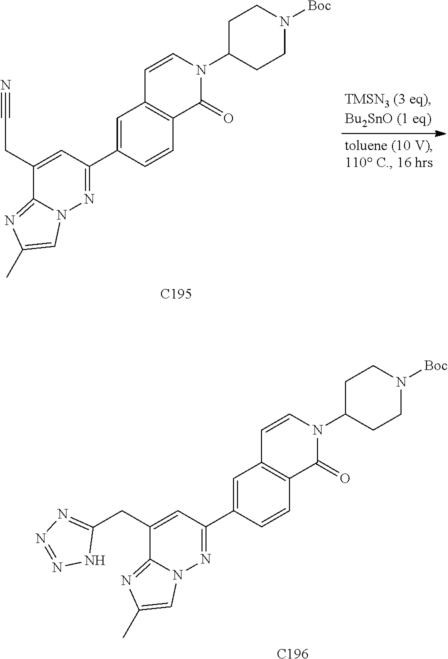 [SMILES: Cc1cn2nc(-c3ccc4c(=O)n(C5CCN(C(=O)OC(C)(C)C)CC5)ccc4c3)cc(CC#N)c2n1 ; "C195" ; Cc1cn2nc(-c3ccc4c(=O)n(C5CCN(C(=O)OC(C)(C)C)CC5)ccc4c3)cc(Cc3nnn[nH]3)c2n1 ; "C196"]

To a mixture of tert-butyl 4-[6-[8-(cyanomethyl)-2-methyl-imidazo[1,2-b]pyridazin-6-yl]-1-oxo-2-isoquinolyl]piperidine-1-carboxylate (100 mg, 200 umol, 1 eq) in toluene (2.00 mL) was added dibutyl(oxo)tin (49.9 mg, 200 umol, 1 eq) and trimethylsilazide (69.3 mg, 601 umol, 3 eq) at 20° C. The mixture was stirred at 110° C. and stirred for 16 hrs. The mixture was cooled to 20° C., poured into ice-water (w/w=1/1, 10.0 mL) and stirred for 30 min. The aqueous phase was extracted with ethyl acetate (3×3 mL). The combined organic phase was washed with brine (3×3 mL), dried with anhydrous Na₂SO₄, filtered and concentrated in vacuum. The residue was purified by prep-HPLC (Condition 15, Gradient 1) to afford tert-butyl 4-[6-[2-methyl-8-(1H-tetrazol-5-ylmethyl)imidazo[1,2-b]pyridazin-6-yl]-1-oxo-2-isoquinolyl]piperidine-1-carboxylate (50.0 mg, 46%) as a solid. LCMS: (ESI, m/z): 542.1 [M+H]⁺. ¹H NMR (400 MHz, MeOD-d₄) δ ppm 8.45 (d, J=8.31 Hz, 1H) 8.27 (d, J=1.47 Hz, 1H) 8.18 (dd, J=8.56, 1.71 Hz, 1H) 8.05 (s, 1H) 7.80 (s, 1H) 7.51 (d, J=7.34 Hz, 1H) 6.84 (d, J=7.82 Hz, 1H) 5.04-5.15 (m, 2H) 4.76 (s, 1H) 4.28-4.36 (m, 2H) 2.91-3.06 (m, 2H) 2.50 (s, 3H) 1.87-1.94 (m, 4H) 1.49 (s, 9H)

### Synthesis of Compound 520

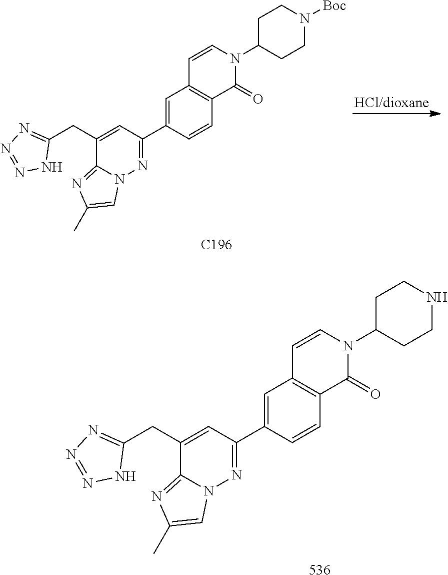 [SMILES: Cc1cn2nc(-c3ccc4c(=O)n(C5CCN(C(=O)OC(C)(C)C)CC5)ccc4c3)cc(Cc3nnn[nH]3)c2n1 ; "C196" ; Cc1cn2nc(-c3ccc4c(=O)n(C5CCNCC5)ccc4c3)cc(Cc3nnn[nH]3)c2n1 ; "536"]

To a solution of tert-butyl 4-[6-[2-methyl-8-(1H-tetrazol-5-ylmethyl) imidazo[1,2-b]pyridazin-6-yl]-1-oxo-2-isoquinolyl]piperidine-1-carboxylate (50.0 mg, 92.3 umol, 1 eq) in dioxane (1.00 mL) was added HCl/dioxane (2 M, 1.00 mL) at 20° C. The reaction mixture was stirred to 20° C. for 2 hrs and concentrated in vacuo. The residue was triturated with ethyl acetate (5.00 mL) to afford 6-[2-methyl-8-(1H-tetrazol-5-ylmethyl) imidazo[1,2-b]pyridazin-6-yl]-2-(4-piperidyl) isoquinolin-1-one (7.23 mg, 18%) as a solid. LCMS: (ESI, m/z): 442.2 [M+H]⁺. ¹H NMR (400 MHz, MeOD-d₄) δ ppm 8.50-8.55 (m, 1H) 8.38-8.47 (m, 3H) 8.27 (dd, J=8.58, 1.91 Hz, 1H) 7.49 (d, J=7.63 Hz, 1H) 6.92 (d, J=7.63 Hz, 1H) 5.19 (s, 1H) 4.88 (s, 2H) 3.58-3.66 (m, 2H) 3.24-3.30 (m, 2H) 2.64-2.66 (m, 3H) 2.33 (qd, J=12.72, 3.34 Hz, 2H) 2.14-2.22 (m, 2H)

### Example 183: Synthesis of Compound 529

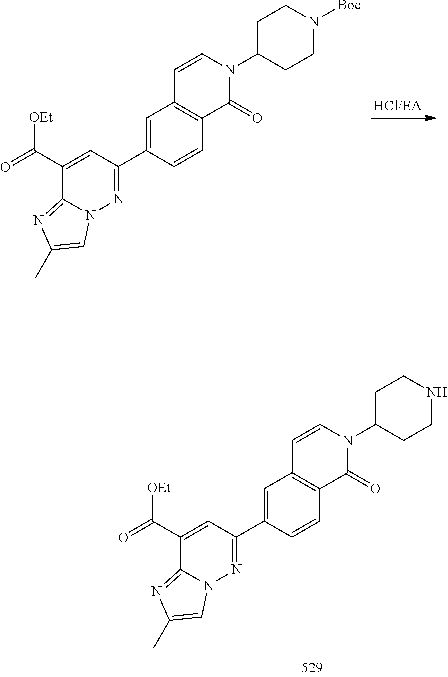 [SMILES: CCOC(=O)c1cc(-c2ccc3c(=O)n(C4CCN(C(=O)OC(C)(C)C)CC4)ccc3c2)nn2cc(C)nc12 ; CCOC(=O)c1cc(-c2ccc3c(=O)n(C4CCNCC4)ccc3c2)nn2cc(C)nc12]

To a mixture of ethyl 6-[2-(1-tert-butoxycarbonyl-4-piperidyl)-1-oxo-6-isoquinolyl]-2-methyl-imidazo[1,2-b]pyridazine-8-carboxylate (30.0 mg, 56.4 umol, 1 eq) in ethyl acetate (1.00 mL) was added HCl/ethyl acetate (2 M, 1.00 mL) at 20° C. The mixture was stirred at 20° C. for 2 hrs. The mixture was concentrated to give a residue, which was triturated with methyl tert-butyl ether (3.00 mL). The solid was filtered and the filter cake was dried to give ethyl 2-methyl-6-[1-oxo-2-(4-piperidyl)-6-isoquinolyl]imidazo[1,2-b]pyridazine-8-carboxylate (7.08 mg, 29%) as a solid. LCMS: (ESI, m/z): 404.1 [M+H]⁺. ¹H NMR (400 MHz, MeOD-d₄) δ ppm 8.89 (s, 1H) 8.49-8.57 (m, 3H) 8.33 (dd, J=8.50, 1.50 Hz, 1H) 7.54 (d, J=7.50 Hz, 1H) 6.96 (d, J=7.50 Hz, 1H) 5.11-5.22 (m, 1H) 3.63 (br d, J=13.01 Hz, 2H) 3.27 (br s, 1H) 2.71 (s, 3H) 2.38 (qd, J=13.01, 4.00 Hz, 2H) 2.18 (br d, J=13.51 Hz, 2H)

### Example 184: Synthesis of Compound 548

### Synthesis of Intermediate C197

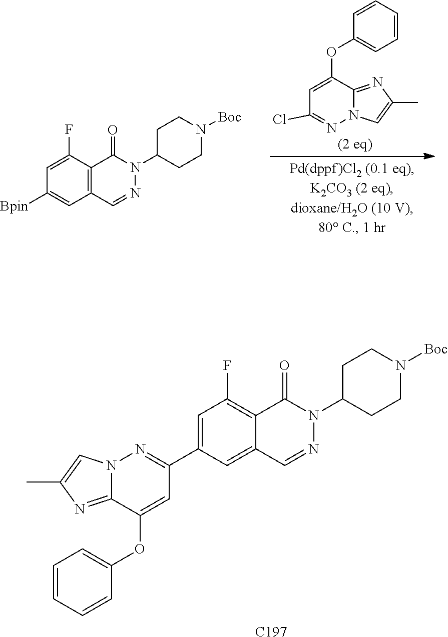

To a mixture of tert-butyl 4-[8-fluoro-1-oxo-6-(4,4,5,5-tetramethyl-1,3,2-dioxaborolan-2-yl)phthalazin-2-yl]piperidine-1-carboxylate (220 mg, 465 umol, 1.5 eq) and 6-chloro-2-methyl-8-phenoxy-imidazo[1,2-b]pyridazine (80.5 mg, 309 umol, 1 eq) in dioxane (400 uL) was added a solution of K₂CO₃ (85.7 mg, 619 umol, 2 eq) in H₂O (100 uL). Then, 1,1-bis(diphenylphosphino)ferrocene]dichloropalladium(II) (22.7 mg, 30.9 umol, 0.1 eq) was added to the above mixture under N₂ protection. The reaction mixture was stirred at 80° C. for 1 hr. The reaction mixture was quenched by additional of water (10.0 mL). The mixture was extracted with ethyl acetate (3×10 mL). The combined organic phase was washed with brine (3×10 mL), dried over Na₂SO₄, filtered and concentrated under reduced pressure to give an oil. The oil was purified by column chromatography on silica gel and eluted with petroleum ether/ethyl acetate (I/O to 5/1) to give tert-butyl 4-[8-fluoro-6-(2-methyl-8-phenoxy-imidazo[1,2-b]pyridazin-6-yl)-1-oxo-phthalazin-2-yl]piperidine-1-carboxylate (115 mg, 55%) as a solid. LCMS: (ESI, m/z): 571.3 [M+H]⁺. ¹H NMR (400 MHz, CDCl₃) δ=8.17 (d, J=2.1 Hz, 1H) 7.86-7.82 (m, 2H) 7.54 (q, J=7.8 Hz, 3H) 7.44-7.36 (m, 2H) 7.33-7.30 (m, 2H) 5.22-5.09 (m, 1H) 4.40-4.19 (m, 2H) 2.98-2.80 (m, 2H) 2.60 (s, 3H) 2.03-1.94 (m, 2H) 1.84 (br d, J=10.1 Hz, 2H) 1.49 (s, 9H). ¹⁹F NMR (376 MHz, CDCl₃) δ ppm−108.25 (br s, 1F).

### Synthesis of Compound 548

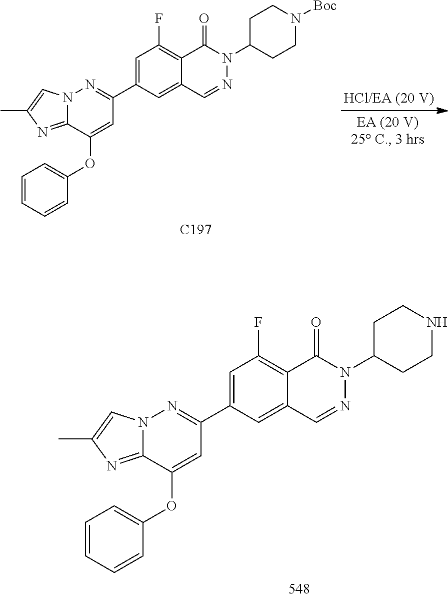 [SMILES: Cc1cn2nc(-c3cc(F)c4c(=O)n(C5CCN(C(=O)OC(C)(C)C)CC5)ncc4c3)cc(Oc3ccccc3)c2n1 ; "C197" ; Cc1cn2nc(-c3cc(F)c4c(=O)n(C5CCNCC5)ncc4c3)cc(Oc3ccccc3)c2n1 ; "548"]

A solution of tert-butyl 4-[8-fluoro-6-(2-methyl-8-phenoxy-imidazo[1,2-b]pyridazin-6-yl)-1-oxo-phthalazin-2-yl]piperidine-1-carboxylate (110 mg, 193 umol, 1 eq) in ethyl acetate (2.20 mL) was added HCl/ethyl acetate (4 M, 1.93 mL). The reaction mixture was stirred at 25° C. for 3 hrs. The reaction mixture was filtered, and the filter cake was purified by prep-HPLC (Condition 13, Gradient 1) to afford 8-fluoro-6-(2-methyl-8-phenoxy-imidazo[1,2-b]pyridazin-6-yl)-2-(4-piperidyl)phthala-zin-1-one (26.3 mg, 29%) as a solid. LCMS: (ESI, m/z): 471.1 [M+H]^{+ 1}H NMR (400 MHz, D₂0) 6=8.31 (s, 1H) 7.92 (s, 1H) 7.87-7.77 (m, 2H) 7.68-7.59 (m, 2H) 7.51 (br d, J=7.6 Hz, 1H) 7.36 (br d, J=7.8 Hz, 2H) 6.82 (s, 1H) 5.22-5.12 (m, 1H) 3.62 (br d, J=13.0 Hz, 2H) 3.36-3.22 (m, 2H) 2.49 (s, 3H) 2.33-2.13 (m, 4H). ¹⁹F NMR (376 MHz, D₂O) δ ppm−110.51 (s, 1F)

### Example 185: Synthesis of Compound 549

### Synthesis of Intermediate C199

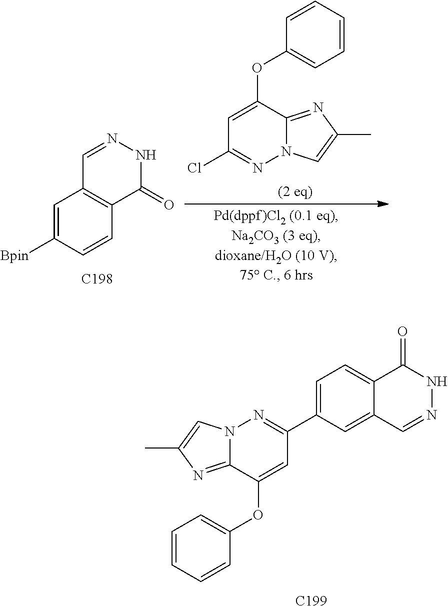

To a mixture of 6-chloro-2-methyl-8-phenoxy-imidazo[1,2-b]pyridazine (EVAL-0116-A14, 50.0 mg, 192 umol, 1 eq) and 6-(4,4,5,5-tetramethyl-1,3,2-dioxaborolan-2-yl)-2H-phthalazin-1-one (78.6 mg, 288 umol, 1.5 eq) in dioxane (0.4 mL) and water (0.1 mL) was added [1,1-bis(diphenylphosphino)ferrocene]dichloropalladium(II) (14.0 mg, 19.2 umol, 0.1 eq) and Na₂CO₃ (61.2 mg, 577 umol, 3 eq) in one portion at 20° C. under N₂ protection. The mixture was stirred at 75° C. and stirred for 6 hours. After cooling to 20° C., the reaction mixture was concentrated to give a residue, which was purified by silica gel chromatography (Petroleum ether/Ethyl acetate=0/1) to afford 6-(2-methyl-8-phenoxy-imidazo[1,2-b]pyridazin-6-yl)-2H-phthalazin-1-one (50.0 mg, 70%) as a solid. LCMS: (ES, m/z): 370.0 [M+H]⁺. ¹H NMR (400 MHz, DMSO-d₆) δ ppm 8.40-8.47 (m, 2H) 8.27-8.33 (m, 2H) 8.20 (s, 1H) 7.51-7.58 (m, 2H) 7.35-7.40 (m, 3H) 6.96 (s, 1H) 4.03 (s, 2H) 3.28-3.34 (m, 1H) 2.43 (s, 3H)

### Synthesis of Intermediate C120

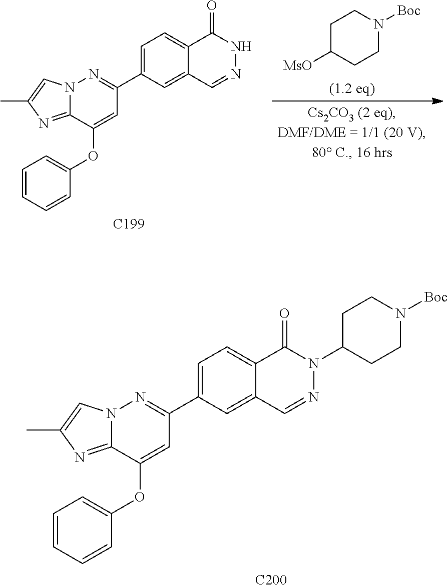 [SMILES: Cc1cn2nc(-c3ccc4c(=O)[nH]ncc4c3)cc(Oc3ccccc3)c2n1 ; CC(C)(C)OC(=O)N1CCC(OS(C)(=O)=O)CC1 ; "C199" ; Cc1cn2nc(-c3ccc4c(=O)n(C5CCN(C(=O)OC(C)(C)C)CC5)ncc4c3)cc(Oc3ccccc3)c2n1 ; "C200"]

To a mixture of 6-(2-methyl-8-phenoxy-imidazo[1,2-b]pyridazin-6-yl)-2H-phthalazin-1-one (50.0 mg, 135 umol, 1 eq) and tert-butyl 4-methylsulfonyloxypiperidine-1-carboxylate (45.3 mg, 162 umol, 1.2 eq) in N, N-dimethyl formamide (0.5 mL) and ethylene glycol dimethyl ether (0.5 mL) was added Cs₂CO₃ (88.2 mg, 270 umol, 2 eq) in one portion at 20° C. under N₂ protection. The mixture was stirred at 20° C. for 30 min, then heated to 80° C. and stirred for 16 hours. After cooling to 20° C., the reaction mixture was concentrated to give a residue, which was poured into ice-water (w/w=1/1) (5 mL) and stirred for 30 min. The aqueous phase was extracted with ethyl acetate (5 mL×3). The combined organic phase was washed with brine (5 mL×3), dried with anhydrous Na₂SO₄, filtered and concentrated in vacuum. The residue was purified by silica gel chromatography (petroleum ether/ethyl acetate=0/1) to afford tert-butyl 4-[6-(2-methyl-8-phenoxy-imidazo[1,2-b]pyridazin-6-yl)-1-oxo-phthalazin-2-yl]piperidine-1-carboxylate (50.0 mg, 67%) as a solid. LCMS: (ES, m/z): 553.1 [M+H]⁺. ¹H NMR (400 MHz, DMSO-d₆) δ ppm 11.69 (s, 1H) 11.66-11.71 (m, 1H) 8.57 (s, 1H) 8.43 (d, J=1.50 Hz, 1H) 8.29-8.38 (m, 2H) 8.21 (s, 1H) 7.52-7.59 (m, 2H) 7.34-7.41 (m, 3H) 6.97 (s, 1H) 5.37 (dd, J=6.00, 2.00 Hz, 1H) 5.08 (br t, J=5.00 Hz, 1H) 4.01-4.15 (m, 2H) 3.75-3.84 (m, 1H) 2.85-3.01 (m, 2H) 2.43 (s, 3H) 1.74-1.85 (m, 4H) 1.42 (s, 9H)

### Synthesis of Compound 549

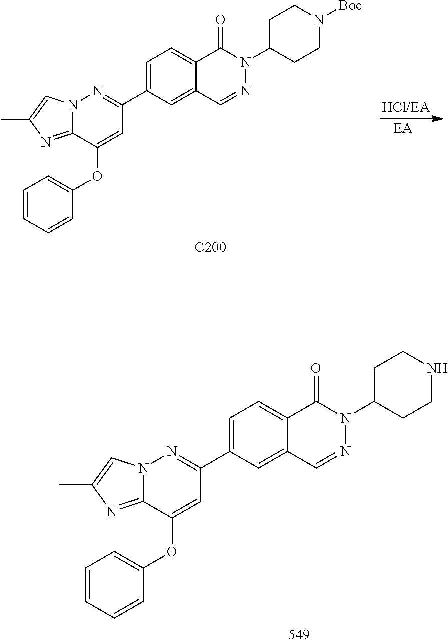 [SMILES: Cc1cn2nc(-c3ccc4c(=O)n(C5CCN(C(=O)OC(C)(C)C)CC5)ncc4c3)cc(Oc3ccccc3)c2n1 ; "C200" ; Cc1cn2nc(-c3ccc4c(=O)n(C5CCNCC5)ncc4c3)cc(Oc3ccccc3)c2n1 ; "549"]

To a mixture of tert-butyl 4-[6-(2-methyl-8-phenoxy-imidazo[1,2-b]pyridazin-6-yl)-1-oxo-phthalazin-2-yl]piperidine-1-carboxylate (50.0 mg, 90.5 umol, 1 eq) in ethyl acetate (1.00 mL) was added HCl/ethyl acetate (2 M, 1.00 mL) at 20° C. The mixture was stirred at 20° C. for 2 hrs. The mixture was concentrated under reduced pressure at 45° C. The residue was purified by silica gel chromatography (petroleum ether/ethyl acetate=0/1) to afford 6-(2-methyl-8-phenoxy-imidazo[1,2-b]pyridazin-6-yl)-2-(4-piperidyl) phthalazin-1-one (25.9 mg, 63%) as a solid. LCMS: (ESI, m/z): 453.1 [M+H]⁺. ¹H NMR (400 MHz, MeOD-d₄) δ ppm 8.49-8.51 (m, 2H) 8.44 (d, J=0.98 Hz, 1H) 8.38-8.43 (m, 1H) 8.34 (s, 1H) 7.62-7.67 (m, 2H) 7.44-7.52 (m, 3H) 7.26 (s, 1H) 5.25-5.35 (m, 1H) 3.59 (br d, J=13.20 Hz, 2H) 3.24-3.30 (m, 2H) 2.66 (s, 3H) 2.30-2.42 (m, 2H) 2.16 (br d, J=13.69 Hz, 2H)

### Example 186: Synthesis of Compound 555

### Synthesis of Intermediate C201

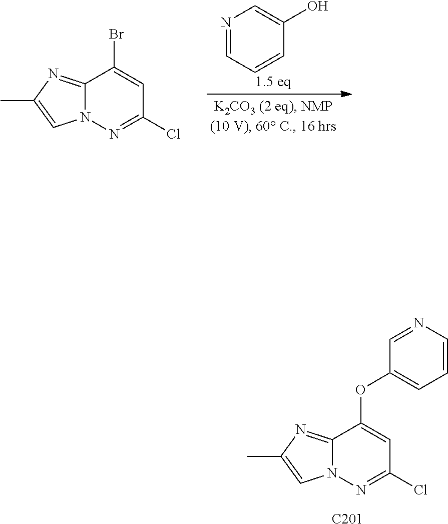 [SMILES: Cc1cn2nc(Cl)cc(Br)c2n1 ; Oc1cccnc1 ; Cc1cn2nc(Cl)cc(Oc3cccnc3)c2n1 ; "C201"]

To a solution of pyridin-3-ol (116 mg, 1.22 mmol, 1.5 eq) and 8-bromo-6-chloro-2-methyl-imidazo[1,2-b]pyridazine (200 mg, 811 umol, 1 eq) in N-methyl pinotanne (2.00 mL) was added K₂CO₃ (224 mg, 1.62 mmol, 2 eq) at 20° C. The reaction mixture was stirred at 60° C. for 16 hrs. The mixture was cooled to 20° C. The reaction mixture was quenched by ice slowly and then extracted with ethyl acetate (3×5 mL). The combined organic phase was washed with brine (3×5 mL), dried over anhydrous Na₂SO₄, filtered and concentrated in vacuum. The residue was purified by silica gel column chromatography (petroleum ether/ethyl acetate=1:1) to afford 6-chloro-2-methyl-8-(3-pyridyloxy)imidazo[1,2-b]pyridazine (100 mg, 47%) as a solid. LCMS: (ESI, m/z): 261.0 [M+H]⁺. ¹H NMR (400 MHz, CDCl₃) δ ppm 8.57-8.69 (m, 2H) 7.73 (s, 1H) 7.61 (ddd, J=8.31, 2.93, 1.47 Hz, 1H) 7.48 (dd, J=8.31, 4.89 Hz, 1H) 6.12 (s, 1H) 2.53 (s, 3H)

### Synthesis of Intermediate C202

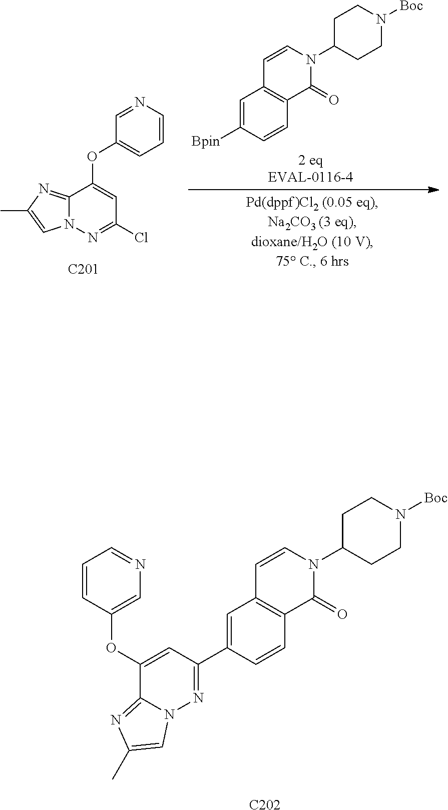

To a mixture of 6-chloro-2-methyl-8-(3-pyridyloxy)imidazo[1,2-b]pyridazine (50.0 mg, 191 umol, 1 eq) and tert-butyl 4-[1-oxo-6-(4,4,5,5-tetramethyl-1,3,2-dioxaborolan-2-yl)-2-isoquinolyl]piperidine-1-carboxylate (131 mg, 287 umol, 1.5 eq) in dioxane (400 uL) and water (100 uL) was added [1,1-bis(diphenylphosphino)ferrocene]dichloropalladium(II) (14.0 mg, 19.1 umol, 0.1 eq) and Na₂CO₃ (40.6 mg, 383 umol, 2 eq) in one portion at 20° C. under N₂ protection. The mixture was stirred at 75° C. and stirred for 6 hours. After cooling to 20° C., the reaction mixture was concentrated to give a residue, which was triturated with methyl tert-butyl ether (5.00 mL) to afford tert-butyl 4-[6-[2-methyl-8-(3-pyridyloxy)imidazo[1,2-b]pyridazin-6-yl]-1-oxo-2isoquinolyl]piperidine-1-carboxylate (15.0 mg, 15%) as a solid. LCMS: (ESI, m/z): 553.2 [M+H]⁺. ¹H NMR (400 MHz, DMSO-d₆) δ ppm 8.68 (d, J=2.93 Hz, 1H) 8.56 (d, J=4.40 Hz, 1H) 8.33 (d, J=8.80 Hz, 1H) 8.18-8.26 (m, 2H) 8.04-8.08 (m, 1H) 7.82-7.87 (m, 1H) 7.62 (d, J=7.82 Hz, 1H) 7.56 (dd, J=8.56, 4.65 Hz, 1H) 7.17 (s, 1H) 6.75 (d, J=7.83 Hz, 1H) 4.98 (br s, 1H) 4.06-4.23 (m, 2H) 2.80-3.03 (m, 2H) 2.41 (s, 3H) 1.71-1.88 (m, 4H) 1.43 (s, 9H)

### Synthesis of Compound 550

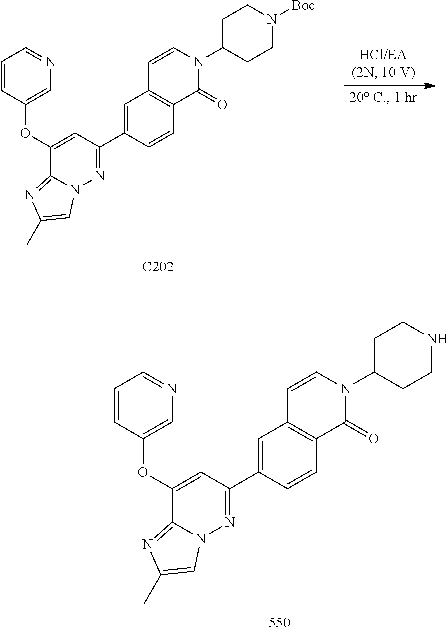 [SMILES: Cc1cn2nc(-c3ccc4c(=O)n(C5CCN(C(=O)OC(C)(C)C)CC5)ccc4c3)cc(Oc3cccnc3)c2n1 ; "C202" ; Cc1cn2nc(-c3ccc4c(=O)n(C5CCNCC5)ccc4c3)cc(Oc3cccnc3)c2n1]

To a solution of tert-butyl 4-[6-[2-methyl-8-(3-pyridyloxy)imidazo[1,2-b]pyridazin-6-yl]-1-oxo-2-isoquinolyl]piperidine-1-carboxylate (15.0 mg, 27.1 umol, 1 eq) in ethyl acetate (500 uL) was added HCl/ethyl acetate (2 M, 500 uL) at 20° C. The mixture was stirred at 20° C. and stirred for 2 hrs. The mixture was concentrated under reduced pressure at 45° C. The residue was purified by prep-HPLC (Condition 13, Gradient 2) to afford 6-[2-methyl-8-(3-pyridyloxy) imidazo[1,2-b]pyridazin-6-yl]-2-(4-piperidyl)isoquinolin-1-one (4.13 mg, 34%) as a solid. LCMS: (ESI, m/z): 453.1 [M+H]⁺. ¹H NMR (400 MHz, MeOD-d₄) δ ppm 8.91 (s, 1H) 8.71-8.78 (m, 1H) 8.39-8.49 (m, 2H) 8.27-8.30 (m, 1H) 8.13 (s, 2H) 7.80-7.87 (m, 1H) 7.76-7.95 (m, 1H) 7.62-7.63 (m, 1H) 7.62 (s, 1H) 7.44 (d, J=7.82 Hz, 1H) 6.82-6.88 (m, 1H) 5.03-5.19 (m, 1H) 3.65 (s, 2H) 3.27 (br d, J=3.42 Hz, 2H) 2.68 (d, J=0.98 Hz, 3H) 2.24-2.39 (m, 2H) 2.12-2.19 (m, 2H)

### Example 187: Synthesis of Compound 551

### Synthesis of Intermediate C203

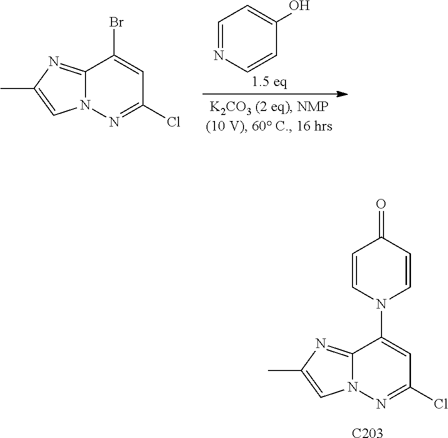 [SMILES: Cc1cn2nc(Cl)cc(Br)c2n1 ; Oc1ccncc1 ; Cc1cn2nc(Cl)cc(-n3ccc(=O)cc3)c2n1 ; "C203"]

To a solution of pyridin-4-ol (115 mg, 1.22 mmol, 1.5 eq) and 8-bromo-6-chloro-2-methyl-imidazo[1,2-b]pyridazine (EVAL-0116-7b, 200 mg, 811 umol, 1 eq) in NMP (2.00 mL) was added K₂CO₃ (112 mg, 811 umol, 1 eq) at 20° C. The reaction mixture was stirred at 60° C. for 12 hrs. The reaction was quenched by ice slowly and then extracted with ethyl acetate (3×5 mL). The combined organic phase was washed with brine (3×5 mL), dried over anhydrous Na₂SO₄, filtered and concentrated in vacuum. The residue was purified by silica gel column chromatography (petroleum ether/ethyl acetate=1:1) to afford 1-(6-chloro-2-methyl-imidazo[1,2-b]pyridazin-8-yl) pyridin-4-one (200 mg, 95%) as a solid. LCMS: (ESI, m/z): 277.9 [M+H]⁺. ¹H NMR (400 MHz, DMSO-d₆) δ ppm 8.51-8.55 (m, 2H) 8.27 (s, 1H) 7.69 (s, 1H) 6.33-6.38 (m, 2H) 2.43 (s, 3H)

### Synthesis of Intermediate C204

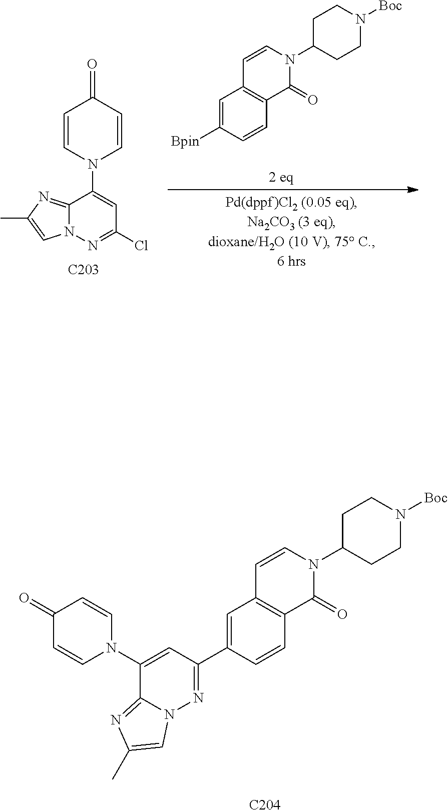

To a mixture of 1-(6-chloro-2-methyl-imidazo[1,2-b]pyridazin-8-yl)pyridin-4-one (50.0 mg, 191 umol, 1 eq) and tert-butyl 4-[1-oxo-6-(4,4,5,5-tetramethyl-1,3,2-dioxaborolan-2-yl)-2-isoquinolyl]piperidine-1-carboxylate (130 mg, 1.5 eq) in dioxane (400 uL) and water (100 uL) was added [1,1-bis(diphenylphosphino)ferrocene]dichloropalladium(II) (14.0 mg, 19.1 umol, 0.1 eq) and Na₂CO₃ (40.6 mg, 383 umol, 2 eq) in one portion at 20° C. under N₂ protection. The mixture was stirred at 75° C. stirred for 6 hrs. After cooling to 20° C., the reaction mixture was concentrated to give a residue, which was triturated with water and ethyl acetate (1/1) to afford tert-butyl 4-[6-[2-methyl-8-(4-oxo-1-pyridyl)imidazo[1,2-b]pyridazin-6-yl]-1-oxo-2-isoquinolyl]piperidine-1-carboxylate (100 mg, 94%) as a solid. LCMS: (ESI, m/z): 553.5 [M+H]⁺. ¹H NMR (400 MHz, DMSO-d₆) δ ppm 8.61-8.64 (m, 2H) 8.47 (d, J=1.47 Hz, 2H) 8.34 (s, 1H) 8.27-8.28 (m, 1H) 8.07 (s, 1H) 7.66 (d, J=7.82 Hz, 1H) 6.76 (d, J=7.34 Hz, 1H) 6.35-6.45 (m, 2H) 4.95-5.06 (m, 1H) 4.14 (br dd, J=9.05, 1.71 Hz, 2H) 2.83-3.04 (m, 2H) 2.47 (s, 3H) 1.74-1.89 (m, 4H) 1.44 (s, 9H)

### Synthesis of Compound 551

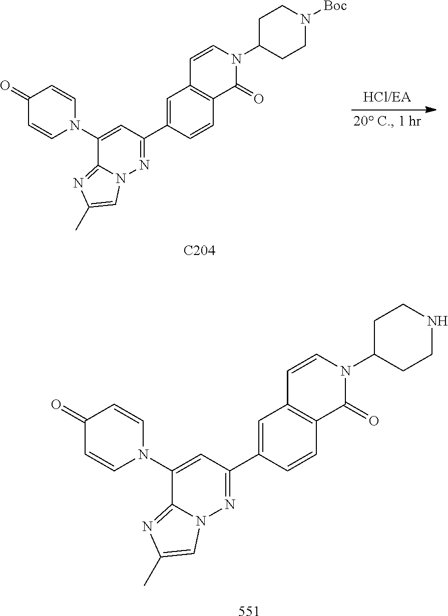 [SMILES: Cc1cn2nc(-c3ccc4c(=O)n(C5CCN(C(=O)OC(C)(C)C)CC5)ccc4c3)cc(-n3ccc(=O)cc3)c2n1 ; "C204" ; Cc1cn2nc(-c3ccc4c(=O)n(C5CCNCC5)ccc4c3)cc(-n3ccc(=O)cc3)c2n1 ; "551"]

To a mixture of tert-butyl 4-[6-[2-methyl-8-(4-oxo-1-pyridyl)imidazo[1,2-b]pyridazin-6-yl]-1-oxo-2-isoquinolyl]piperidine-1-carboxylate (50.0 mg, 90.4 umol, 1 eq) in ethyl acetate (1.00 mL) was added HCl/ethyl acetate (2 M, 1.00 mL) at 20° C. The mixture was stirred at 20° C. for 1 hr. The mixture was concentrated in reduced pressure at 45° C. The crude product was triturated with water and acetonitrile (1/1) to afford 6-[2-methyl-8-(4-oxo-1-pyridyl)imidazo[1,2-b]pyridazin-6-yl]-2-(4-piperidyl)isoquinolin-1-one (4.13 mg, 10%) as a solid. LCMS: (ESI, m/z): 453.1 [M+H]⁺. ¹H NMR (400 MHz, MeOD-d₄) δ ppm 8.74-8.80 (m, 2H) 8.49 (d, J=8.50 Hz, 1H) 8.42 (d, J=1.50 Hz, 1H) 8.29 (dd, J=8.50, 2.00 Hz, 1H) 8.22 (s, 1H) 8.10 (s, 1H) 7.46 (d, J=7.50 Hz, 1H) 6.83-6.93 (m, 3H) 5.07-5.20 (m, 1H) 3.61 (br d, J=13.01 Hz, 2H) 3.26 (br s, 2H) 2.54 (s, 3H) 2.25-2.39 (m, 2H) 2.14-2.22 (m, 2H)

### Example 188: Synthesis of Compound 544

### Synthesis of Intermediate C205

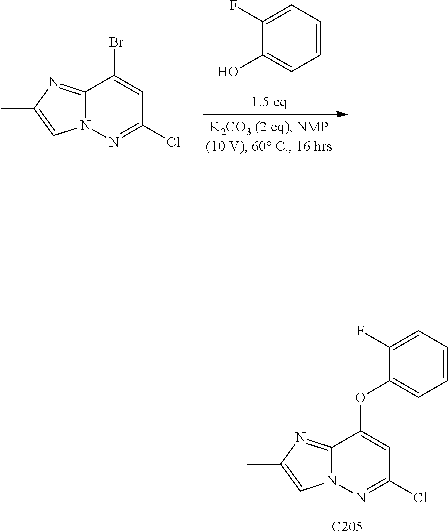 [SMILES: Cc1cn2nc(Cl)cc(Br)c2n1 ; Oc1ccccc1F ; Cc1cn2nc(Cl)cc(Oc3ccccc3F)c2n1 ; "C205"]

To a solution of 2-fluorophenol (136 mg, 1.22 mmol, 113 μL, 1.5 eq) and 8-bromo-6-chloro-2-methyl-imidazo[1,2-b]pyridazine (EVAL-0116-7b, 200 mg, 811 umol, 1 eq) in N-methyl pentanone (2.00 mL) was added K₂CO₃ (224 mg, 1.62 mmol, 2 eq) at 20° C. The reaction mixture was stirred at 60° C. for 16 hrs. The reaction was quenched by water and then extracted with ethyl acetate (3×5 mL). The combined organic phase was washed with brine (3×5 mL), dried over anhydrous Na₂SO₄, filtered and concentrated in vacuum. The residue was purified by silica gel column chromatography (petroleum ether/ethyl acetate=1:1)) to afford 6-chloro-8-(2-fluorophenoxy)-2-methyl-imidazo[1,2-b]pyridazine (100 mg, 44%) as a solid. LCMS: (ESI, m/z): 278.0 [M+H]⁺. ¹H NMR (400 MHz, CDCl₃) δ ppm 7.71 (d, J=2.50 Hz, 1H) 7.28-7.40 (m, 4H) 6.08 (d, J=2.00 Hz, 1H) 2.53 (d, J=3.00 Hz, 3H) ¹⁹F NMR (400 MHz, CDCl₃) δ ppm−127.79 (s, 1H)

### Synthesis of Intermediate C206

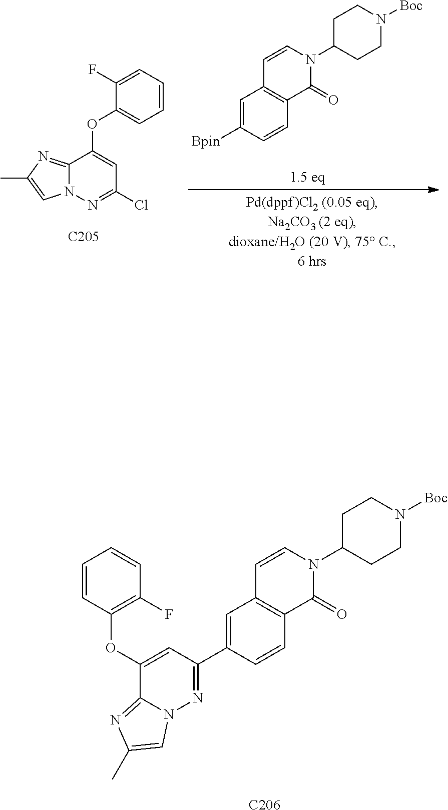

To a mixture of 6-chloro-8-(2-fluorophenoxy)-2-methyl-imidazo[1,2-b]pyridazine (50.0 mg, 180 umol, 1 eq) and tert-butyl 4-[1-oxo-6-(4,4,5,5-tetramethyl-1,3,2-dioxaborolan-2-yl)-2-isoquinolyl]piperidine-1-carboxylate (122 mg, 270 umol, 1.5 eq) in dioxane (750 uL) and water (250 uL) was added[1,1-bis(diphenylphosphino)ferrocene]dichloropalladium(II) (13.1 mg, 18.0 umol, 0.1 eq) and Na₂CO₃ (38.1 mg, 360 umol, 2 eq) in one portion at 20° C. under N₂ protection. The mixture was stirred at 80° C. and stirred for 3 hrs. After cooling to 20° C., the reaction mixture was concentrated to give a residue, which was purified by prep-TLC (Petroleum ether/Ethyl acetate=0/1) to afford tert-butyl 4-[6-[8-(2-fluorophenoxy)-2-methyl-imidazo[1,2-b]pyridazin-6-yl]-1-oxo-2-isoquinolyl]piperidine-1-carboxylate (50.0 mg, 87.8 umol, 49%) as a solid. LCMS: (ESI, m/z): 570.2 [M+H]⁺. ¹H NMR (400 MHz, MeOD-d₄) δ ppm 8.38 (d, J=8.50 Hz, 1H) 8.04 (dd, J=4.75, 1.25 Hz, 2H) 7.96 (dd, J=8.50, 2.00 Hz, 1H) 7.35-7.55 (m, 5H) 6.73-6.80 (m, 2H) 5.03-5.13 (m, 1H) 4.30 (br d, J=13.01 Hz, 2H) 2.90-3.06 (m, 2H) 2.52 (s, 3H) 1.84-1.92 (m, 4H) 1.49 (s, 9H)¹⁹H NMR (376 MHz, MeOD-d₄) δ ppm−131.48 (s, 1F)

### Synthesis of Compound 544

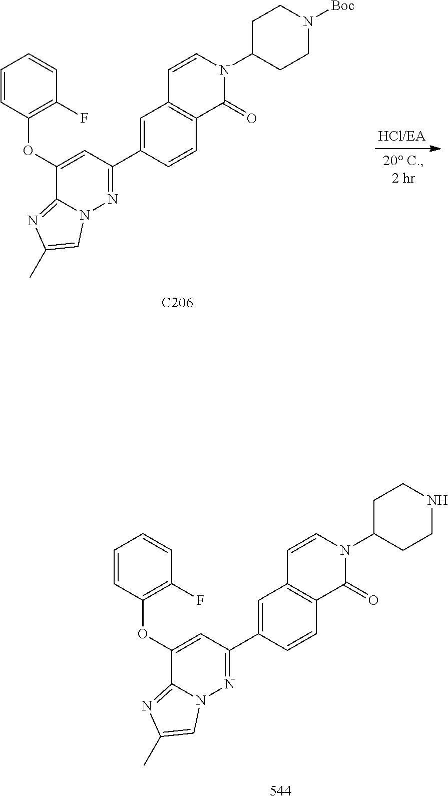 [SMILES: Cc1cn2nc(-c3ccc4c(=O)n(C5CCN(C(=O)OC(C)(C)C)CC5)ccc4c3)cc(Oc3ccccc3F)c2n1 ; "C206" ; Cc1cn2nc(-c3ccc4c(=O)n(C5CCNCC5)ccc4c3)cc(Oc3ccccc3F)c2n1 ; "544"]

To a mixture of tert-butyl 4-[6-[8-(2-fluorophenoxy)-2-methyl-imidazo[1,2-b]pyridazin-6-yl]-1-oxo-2-isoquinolyl]piperidine-1-carboxylate (50.0 mg, 87.8 umol, 1 eq) in ethyl acetate (1.00 mL) was added HCl/ethyl acetate (2 M, 1.00 mL) at 20° C. The mixture was stirred at 20° C. for 2 hrs. The mixture was concentrated to give a residue, which was purified by prep-HPLC (Condition 13, Gradient 3) to afford 6-[8-(2-fluorophenoxy)-2-methyl-imidazo[1,2-b]pyridazin-6-yl]-2-(4-piperidyl) isoquinolin-1-one; hydrochloride (39.3 mg, 89%) as a solid. LCMS: (ESI, m/z): 470.1 [M+H]⁺. ¹H NMR (400 MHz, MeOD-d₄) δ ppm 8.45 (d, J=8.80 Hz, 1H) 8.40 (d, J=0.98 Hz, 1H) 8.23 (d, J=1.96 Hz, 1H) 8.09 (dd, J=8.56, 1.71 Hz, 1H) 7.61 (td, J=7.83, 1.47 Hz, 1H) 7.49-7.57 (m, 2H) 7.41-7.49 (m, 2H) 7.36 (s, 1H) 6.86 (d, J=7.82 Hz, 1H) 5.06-5.19 (m, 1H) 3.61 (br d, J=13.20 Hz, 2H) 3.23-3.29 (m, 2H) 2.69 (s, 3H) 2.32 (qd, J=13.04, 3.91 Hz, 2H) 2.15 (br d, J=13.69 Hz, 2H) ¹⁹F NMR (376 MHz, MeOD-d₄) δ ppm−131.34 (s, 1F)

### Example 189: Synthesis of Compound 545

### Synthesis of Intermediate C207

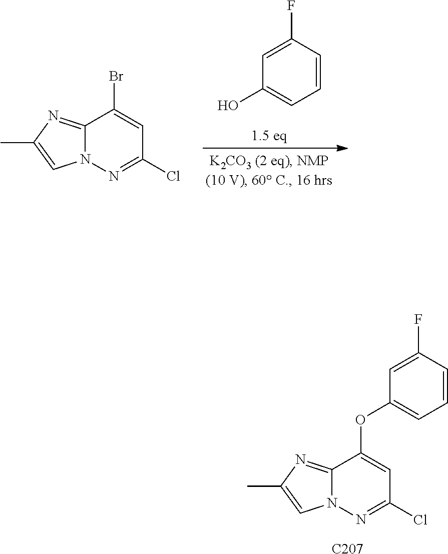 [SMILES: Cc1cn2nc(Cl)cc(Br)c2n1 ; Oc1cccc(F)c1 ; Cc1cn2nc(Cl)cc(Oc3cccc(F)c3)c2n1 ; "C207"]

To a solution of 3-fluorophenol (136 mg, 1.22 mmol, 111 uL, 1.5 eq) and 8-bromo-6-chloro-2-methyl-imidazo[1,2-b]pyridazine (200 mg, 811 umol, 1 eq) in N-methyl-2-pyrrolidone (2.00 mL) was added K₂CO₃ (224 mg, 1.62 mmol, 2 eq) at 20° C. The reaction mixture was heated to 60° C. for 16 hrs. The reaction was quenched by water and then extracted with ethyl acetate (3×5 mL). The combined organic phase was washed with brine (3×5 mL), dried over anhydrous Na₂SO₄, filtered and concentrated in vacuum. The residue was purified by silica gel column chromatography (petroleum ether/ethyl acetate=1:1) to afford 6-chloro-8-(3-fluorophenoxy)-2-methyl-imidazo[1,2-b]pyridazine (100 mg, 44%) as a solid. LCMS: (ESI, m/z): 278.0 [M+H]⁺. ¹H NMR (400 MHz, CDCl₃) δ ppm 7.71 (s, 1H) 7.48 (td, J=8.25, 6.50 Hz, 1H) 7.04-7.12 (m, 2H) 6.99 (dt, J=9.01, 2.50 Hz, 1H) 6.16 (s, 1H) 2.53 (s, 3H) 1⁹F NMR (376 MHz, CDCl₃) δ ppm−108.71 (s, 1F)

### Synthesis of Intermediate C208

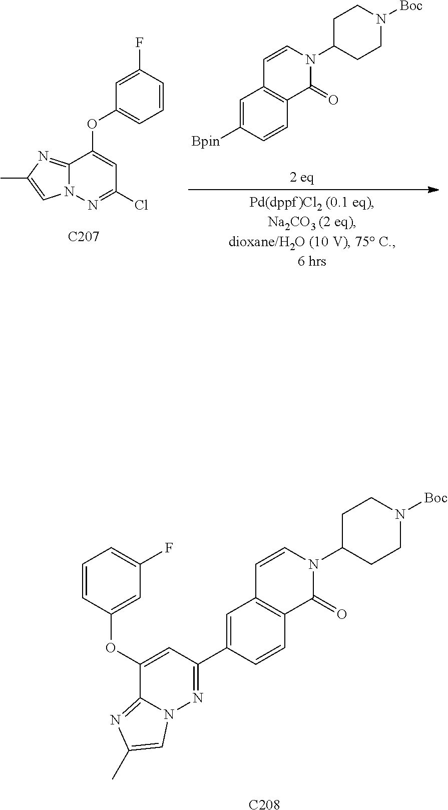

To a mixture of 6-chloro-8-(3-fluorophenoxy)-2-methyl-imidazo[1,2-b]pyridazine (50.0 mg, 180 umol, 1 eq) and tert-butyl 4-[1-oxo-6-(4,4,5,5-tetramethyl-1,3,2-dioxaborolan-2-yl)-2-isoquinolyl]piperidine-1-carboxylate (122 mg, 270 umol, 2 eq) in dioxane (1.00 mL) and water (300 uL) was added [1,1-bis(diphenylphosphino)ferrocene]dichloropalladium(II) (13.1 mg, 18.0 umol, 0.1 eq) and Na₂CO₃ (38.1 mg, 360 umol, 2 eq) in one portion at 20° C. under nitrogen protection. The mixture was stirred at 75° C. for 6 hrs. After cooling to 20° C., the reaction mixture was concentrated to give a residue, which was purified by prep-TLC (petroleum ether/ethyl acetate=0/1) to afford tert-butyl 4-[6-[8-(3-fluorophenoxy)-2-methyl-imidazo[1,2-b]pyridazin-6-yl]-1-oxo-2-isoquinolyl]piperidine-1-carboxylate (60 mg, 58%) as a solid. LCMS: (ESI, m/z): 570.2 [M+H]⁺. ¹H NMR (400 MHz, MeOD-d₄) δ ppm 8.40 (d, J=8.50 Hz, 1H) 8.09 (d, J=1.50 Hz, 1H) 7.99-8.04 (m, 2H) 7.54-7.61 (m, 1H) 7.47 (d, J=7.50 Hz, 1H) 7.12-7.23 (m, 3H) 6.94 (s, 1H) 6.79 (d, J=7.50 Hz, 1H) 5.07-5.10 (m, 1H) 4.30 (br d, J=13.51 Hz, 2H) 2.98 (br dd, J=3.50, 2.50 Hz, 2H) 2.51 (s, 3H) 1.84-1.92 (m, 4H) 1.49 (s, 9H) ¹⁹F NMR (400 MHz, MeOD-d₄) δ ppm−111.41 (s, 1F)

### Synthesis of Compound 545

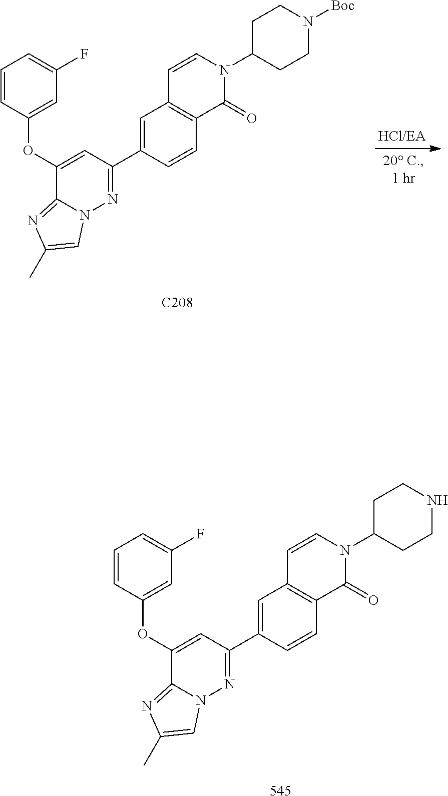 [SMILES: Cc1cn2nc(-c3ccc4c(=O)n(C5CCN(C(=O)OC(C)(C)C)CC5)ccc4c3)cc(Oc3cccc(F)c3)c2n1 ; "C208" ; Cc1cn2nc(-c3ccc4c(=O)n(C5CCNCC5)ccc4c3)cc(Oc3cccc(F)c3)c2n1 ; "545"]

To a mixture of tert-butyl 4-[6-[8-(3-fluorophenoxy)-2-methyl-imidazo[1,2-b]pyridazin-6-yl]-1-oxo-2-isoquinolyl]piperidine-1-carboxylate (50.0 mg, 87.8 umol, 1 eq) in ethyl acetate (1.00 mL) was added HCl/ethyl acetate (2 M, 1.00 mL) at 20° C. The mixture was stirred at 20° C. and stirred for 2 hrs. The mixture was concentrated in reduced pressure at 45° C. The residue was pre-purified by prep-HPLC (Condition 13, Gradient 3) to afford 6-[8-(3-fluorophenoxy)-2-methyl-imidazo[1,2-b]pyridazin-6-yl]-2-(4-piperidyl)isoquinolin-1-one; hydrochloride (27.8 mg, 63%) as a solid. LCMS: (ESI, m/z): 470.1 [M+H]⁺. ¹H NMR (400 MHz, MeOD-d₄) δ ppm 8.42-8.50 (m, 2H) 8.25 (d, J=1.47 Hz, 1H) 8.12 (dd, J=8.56, 1.71 Hz, 1H) 7.67 (td, J=8.31, 6.36 Hz, 1H) 7.46-7.51 (m, 2H) 7.32-7.38 (m, 2H) 7.28 (td, J=8.56, 1.96 Hz, 1H) 6.88 (d, J=7.34 Hz, 1H) 5.08-5.21 (m, 1H) 3.62 (br d, J=13.20 Hz, 2H) 3.24-3.30 (m, 2H) 2.70 (s, 3H) 2.34 (qd, J=13.04, 4.40 Hz, 2H) 2.16 (br d, J=13.69 Hz, 2H) ¹⁹F NMR (376 MHz, MeOD-d₄) δ ppm−110.36 (s, 1F)

### Example 190: Synthesis of Compound 546

### Synthesis of Intermediate C209

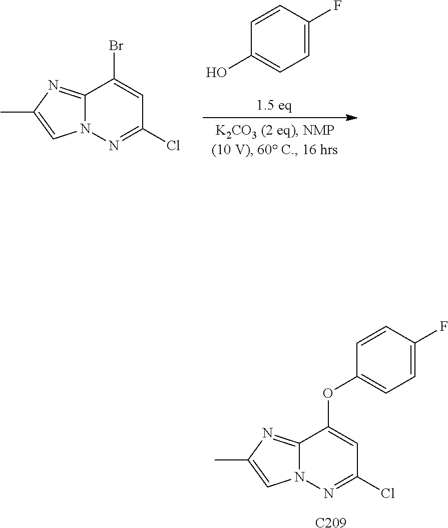 [SMILES: Cc1cn2nc(Cl)cc(Br)c2n1 ; Oc1ccc(F)cc1 ; Cc1cn2nc(Cl)cc(Oc3ccc(F)cc3)c2n1 ; "C209"]

To a solution of 4-fluorophenol (136 mg, 1.22 mmol, 1.5 eq) and 8-bromo-6-chloro-2-methyl-imidazo[1,2-b]pyridazine (200 mg, 811 umol, 1 eq) in N-methyl-2-pyrrolidone (2.00 mL) was added K₂CO₃ (224 mg, 1.62 mmol, 2 eq) at 20° C. The reaction mixture was heated to 60° C. for 12 hrs. The reaction was quenched by ice slowly and then extracted with ethyl acetate (5 mL×3). The combined organic phase was washed with brine (5 mL×3), dried over anhydrous Na₂SO₄, filtered and concentrated in vacuum. The residue was purified by silica gel column chromatography (petroleum ether/ethyl acetate=1:1)) to afford 6-chloro-8-(4-fluorophenoxy)-2-methyl-imidazo[1,2-b]pyridazine (100 mg, 44%) as a solid. LCMS: (ES, m/z): 278.0 [M+H]⁺. ¹H NMR (400 MHz, CHLOROFORM-d) δ ppm 7.70 (s, 1H) 7.17-7.25 (m, 4H) 6.08 (s, 1H) 2.53 (s, 3H)

### Synthesis of Intermediate C210

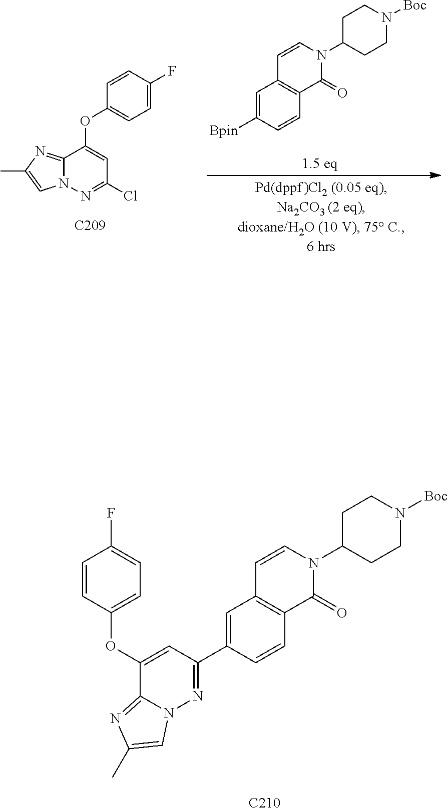

To a mixture of 6-chloro-8-(4-fluorophenoxy)-2-methyl-imidazo[1,2-b]pyridazine (50.0 mg, 180 umol, 1 eq) and tert-butyl 4-[1-oxo-6-(4,4,5,5-tetramethyl-1,3,2-dioxaborolan-2-yl)-2-isoquinolyl]piperidine-1-carboxylate (122 mg, 270 umol, 1.5 eq) in dioxane (400 uL) and water (100 uL) was added [1,1-bis(diphenylphosphino)ferrocene]dichloropalladium(II) (13.1 mg, 18.0 umol, 0.1 eq) and Na₂CO₃ (38.1 mg, 360 umol, 2 eq) in portions at 20° C. under N₂ protection. The mixture was stirred 75° C. and stirred for 6 hrs. After cooling to 20° C., the reaction mixture was concentrated to give a residue, which was purified by prep-TLC (petroleum ether/ethyl acetate=0/1) to afford tert-butyl 4-[6-[8-(4-fluorophenoxy)-2-methyl-imidazo[1,2-b]pyridazin-6-yl]-1-oxo-2-isoquinolyl]piperidine-1-carboxylate (60.0 mg, 59%) as a solid. LCMS: (ESI, m/z): 570.2 [M+H]⁺. ¹H NMR (400 MHz, MeOD-d₄) δ ppm 8.39 (d, J=8.50 Hz, 1H) 8.06 (s, 1H) 7.97-8.03 (m, 2H) 7.45-7.50 (m, 1H) 7.38-7.43 (m, 2H) 7.27-7.34 (m, 2H) 6.76-6.83 (m, 2H) 5.04-5.11 (m, 1H) 4.30 (br d, J=13.01 Hz, 2H) 2.91-3.06 (m, 2H) 2.52 (s, 3H) 1.85-1.90 (m, 4H) 1.49 (s, 9H) ¹⁹F NMR (376 MHz, MeOD-d₄) δ ppm−117.90 (s, 1F)

### Synthesis of Compound 546

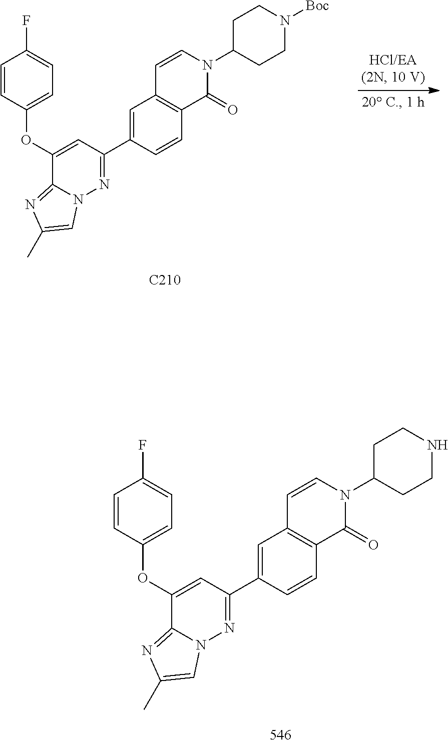 [SMILES: Cc1cn2nc(-c3ccc4c(=O)n(C5CCN(C(=O)OC(C)(C)C)CC5)ccc4c3)cc(Oc3ccc(F)cc3)c2n1 ; "C210" ; Cc1cn2nc(-c3ccc4c(=O)n(C5CCNCC5)ccc4c3)cc(Oc3ccc(F)cc3)c2n1 ; "546"]

To a mixture of tert-butyl 4-[6-[8-(4-fluorophenoxy)-2-methyl-imidazo[1,2-b]pyridazin-6-yl]-1-oxo-2-isoquinolyl]piperidine-1-carboxylate (50.0 mg, 87.7 umol, 1 eq) in ethyl acetate (1.00 mL) was added HCl/ethyl acetate (2 M, 1.00 mL) at 20° C. The mixture was stirred at 20° C. and stirred for 2 hrs. The mixture was concentrated under reduced pressure at 45° C. The residue was purified by prep-HPLC (condition 13, Gradient 3) to afford 6-[8-(4-fluorophenoxy)-2-methyl-imidazo[1,2-b]pyridazin-6-yl]-2-(4-piperidyl) isoquinolin-1-one; hydrochloride (19.5 mg, 44.1%) as a solid. LCMS: (ESI, m/z): 470.1 [M+H]⁺. ¹H NMR (400 MHz, MeOD-d₄) δ ppm 8.45 (d, J=8.31 Hz, 1H) 8.39 (d, J=0.98 Hz, 1H) 8.21 (d, J=1.96 Hz, 1H) 8.09 (dd, J=8.80, 1.96 Hz, 1H) 7.44-7.55 (m, 3H) 7.32-7.42 (m, 3H) 6.86 (d, J=7.34 Hz, 1H) 5.07-5.18 (m, 1H) 3.61 (br d, J=13.20 Hz, 2H) 3.22-3.30 (m, 2H) 2.68 (d, J=0.98 Hz, 3H) 2.27-2.39 (m, 2H) 2.15 (br d, J=13.20 Hz, 2H) ¹⁹F NMR (376 MHz, MeOD-d₄) δ ppm−116.32 (s, 1F)

### Example 191: Synthesis of Compound 547

### Synthesis of Intermediate C211

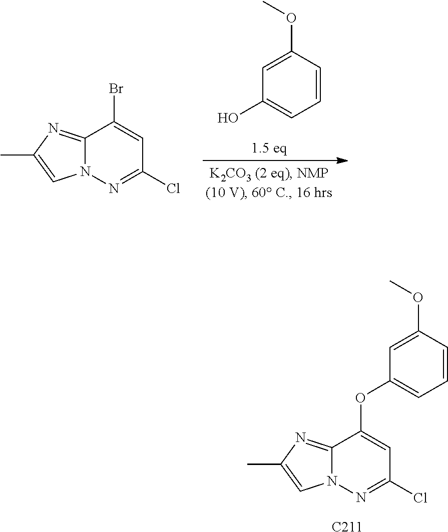

To a solution of 8-bromo-6-chloro-2-methyl-imidazo[1,2-b]pyridazine (200 mg, 811 umol, 1 eq) and 3-methoxyphenol (151 mg, 1.22 mmol, 131 μL, 1.5 eq) in N-methylpyrrolidone (2.00 mL) was added K₂CO₃ (224 mg, 1.62 mmol, 2 eq) at 25° C. under N₂. The reaction mixture was stirred at 60° C. for 16 hrs. TLC (petroleum ether/ethyl acetate=2/1) showed all of the starting material was consumed and a new major spot was generated. The residue was poured into saturated sodium chloride (10.0 mL), and extracted with ethyl acetate (3×10.0 mL). The combined organic phase was washed with brine (10.0 mL), dried with anhydrous Na₂SO₄, filtered and concentrated in vacuo. The crude product was purified by silica gel chromatography (petroleum ether/ethyl acetate=3/1) to give 6-chloro-8-(3-methoxyphenoxy)-2-methyl-imidazo[1,2-b]pyridazine (100 mg, 51.1%) as a solid. LCMS: (ESI, m/z): 290.0 [M+H]^{+ 1}H NMR (400 MHz, METHANOL-d₄) δ ppm 7.87 (d, J=0.73 Hz, 1H) 7.45 (t, J=8.19 Hz, 1H) 6.97 (ddd, J=8.44, 2.32, 0.73 Hz, 1H) 6.85-6.91 (m, 2H) 6.25 (s, 1H) 3.84 (s, 3H) 2.46 (d, J=0.61 Hz, 3H)

### Synthesis of Intermediate C212

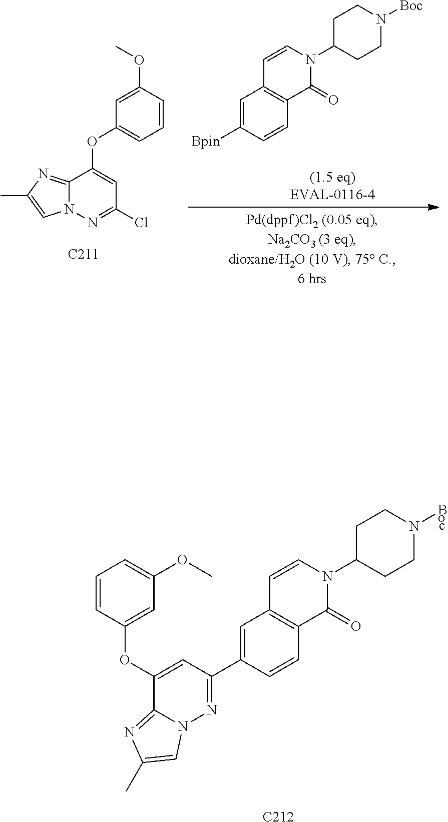

To a solution of 6-chloro-8-(3-methoxyphenoxy)-2-methyl-imidazo[1,2-b]pyridazine (100 mg, 345 umol, 1 eq) and tert-butyl 4-[1-oxo-6-(4,4,5,5-tetramethyl-1,3,2-dioxaborolan-2-yl)-2-isoquinolyl]piperidine-1-carboxylate (235 mg, 518 umol, 1.5 eq) in dioxane (0.80 mL) and H₂O (0.20 mL) was added Na₂CO₃ (109 mg, 1.04 mmol, 3 eq) and [1,1-bis(diphenylphosphino) ferrocene]dichloropalladium (II) (25.3 mg, 34.5 umol, 0.1 eq) at 25° C. under N₂. The reaction mixture was stirred at 75° C. for 6 hrs. TLC (petroleum ether/ethyl acetate=1/1) showed all of the starting material was consumed and a new major spot was generated. The mixture was poured into H₂O (10.0 mL) and the resulting mixture was extracted with ethyl acetate (3×10.0 mL). The combined organic phase was washed with brine (10.0 mL), dried with anhydrous Na₂SO₄, filtered and concentrated in vacuo. The crude product was purified by silica gel chromatography (petroleum ether/ethyl acetate=3/1) to give tert-butyl 4-[6-[8-(3-methoxyphenoxy)-2-methyl-imidazo[1,2-b]pyridazin-6-yl]-1-oxo-2-isoquinolyl]piperidine-1-carboxylate (50.0 mg, 25%) as a solid. LCMS: (ESI, m/z): 582.3 [M+H]^{+ 1}H NMR (400 MHz, MeOD-d₄) δ ppm 8.37 (d, J=8.63 Hz, 1H) 8.01 (dd, J=10.32, 1.06 Hz, 2H) 7.95 (dd, J=8.50, 1.75 Hz, 1H) 7.43-7.49 (m, 2H) 6.90-7.00 (m, 3H) 6.82 (s, 1H) 6.76 (d, J=7.50 Hz, 1H) 5.07 (dt, J=15.85, 7.90 Hz, 1H) 4.30 (br d, J=13.26 Hz, 2H) 3.85 (s, 3H) 2.87-3.07 (m, 2H) 2.51 (s, 3H) 1.83-1.92 (m, 4H) 1.49 (s, 9H)

### Synthesis of Compound 547

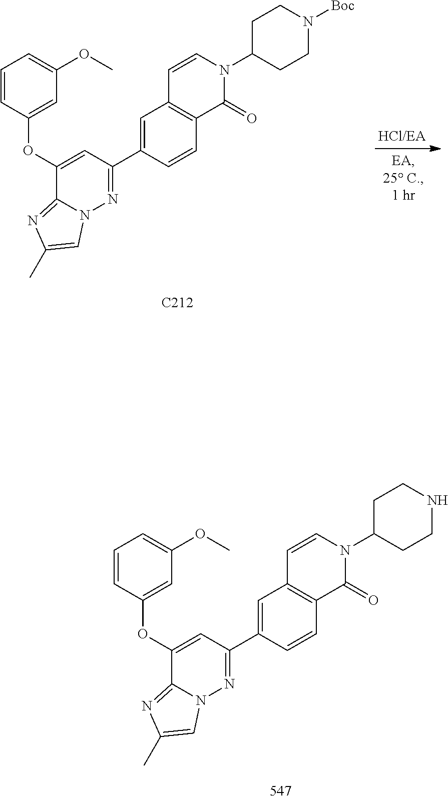 [SMILES: COc1cccc(Oc2cc(-c3ccc4c(=O)n(C5CCN(C(=O)OC(C)(C)C)CC5)ccc4c3)nn3cc(C)nc23)c1 ; "C212" ; COc1cccc(Oc2cc(-c3ccc4c(=O)n(C5CCNCC5)ccc4c3)nn3cc(C)nc23)c1 ; "547"]

To a solution of tert-butyl 4-[6-[8-(3-methoxyphenoxy)-2-methyl-imidazo[1,2-b]pyridazin-6-yl]-1-oxo-2-isoquinolyl]piperidine-1-carboxylate (50.0 mg, 103 umol, 1 eq) in ethyl acetate (1.00 mL) was added HCl/ethyl acetate (4 M, 1.00 mL) at 25° C. under N₂. The reaction mixture was stirred at 25° C. for 1 hr. The reaction mixture was concentrated under reduced pressure to give the crude product. The crude product was triturated with methyl tert-butyl ether (1.00 mL). The solid was filtered and concentrated under reduced pressure to give 6-[8-(3-methoxyphenoxy)-2-methyl-imidazo[1,2-b]pyridazin-6-yl]-2-(4 piperidyl) isoquinolin-1-one (30.0 mg, 59%). LCMS: (ESI, m/z): 482.1 [M+H]⁺. ¹H NMR (400 MHz, MeOD-d₄) δ ppm 8.45 (d, J=8.44 Hz, 1H) 8.38 (s, 1H) 8.19 (d, J=1.35 Hz, 1H) 8.04-8.09 (m, 1H) 7.50-7.56 (m, 1H) 7.46 (d, J=7.70 Hz, 1H) 7.33 (s, 1H) 7.00-7.08 (m, 3H) 6.86 (d, J=7.58 Hz, 1H) 5.10-5.15 (m, 1H) 3.87 (s, 3H) 3.61 (brd, J=12.84 Hz, 2H) 3.22-3.30 (m, 2H) 2.68 (s, 3H) 2.33 (qd, J=13.08, 3.55 Hz, 2H) 2.15 (br d, J=13.33 Hz, 2H)

### Example 192: Synthesis of Compound 552

### Synthesis of Intermediate C213

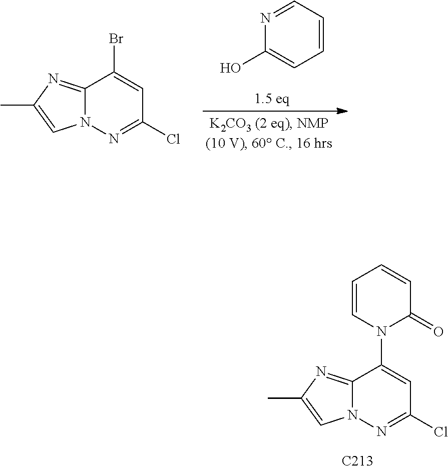 [SMILES: Cc1cn2nc(Cl)cc(Br)c2n1 ; Oc1ccccn1 ; Cc1cn2nc(Cl)cc(-n3ccccc3=O)c2n1 ; "C213"]

To a solution of pyridin-2-ol (115 mg, 1.22 mmol, 1.5 eq) and 8-bromo-6-chloro-2-methyl-imidazo[1,2-b]pyridazine (200 mg, 811 umol, 1 eq) in N-methyl pentanone (2.00 mL) was added K₂CO₃ (224 mg, 1.62 mmol, 2 eq) at 20° C. The reaction mixture was heated to 60° C. for 16 hrs. The reaction was quenched by ice slowly and then extracted with ethyl acetate (3×5 mL). The combined organic phase was washed with brine (3×5 mL), dried over anhydrous Na₂SO₄, filtered and concentrated in vacuum. The residue was purified by silica gel column chromatography (petroleum ether/ethyl acetate=1:1) to afford 1-(6-chloro-2-methyl-imidazo[1,2-b]pyridazin-8-yl) pyridin-2-one (100 mg, 47%) as a solid and 6-chloro-2-methyl-8-(pyridin-2-yloxy)imidazo[1,2-b]pyridazine (30 mg, 15%) as a solid. LCMS: (ESI, m/z): 261.0 [M+H]⁺. ¹H NMR (400 MHz, CDCl₃) δ ppm 8.32 (dd, J=4.89, 1.47 Hz, 1H) 7.87 (ddd, J=8.31, 7.34, 1.96 Hz, 1H) 7.69-7.72 (m, 1H) 7.21-7.26 (m, 2H) 7.04 (s, 1H) 2.48-2.54 (m, 3H)

### Synthesis of Intermediate C214

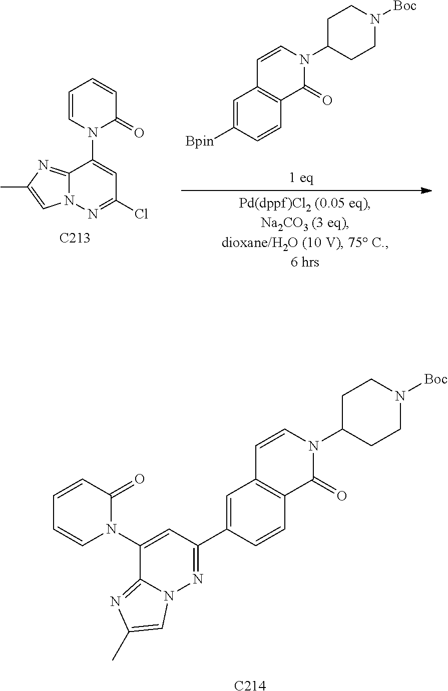

To a mixture of 1-(6-chloro-2-methyl-imidazo[1,2-b]pyridazin-8-yl)pyridin-2-one (50.0 mg, 191 umol, 1 eq) and tert-butyl 4-[1-oxo-6-(4,4,5,5-tetramethyl-1,3,2-dioxaborolan-2-yl)-2-isoquinolyl]piperidine-1-carboxylate (87.1 mg, 194 umol, 1 eq) in dioxane (400 uL) and water (100 uL) was added [1,1-bis(diphenylphosphino)ferrocene]dichloropalladium(II) (7.02 mg, 9.59 umol, 0.05 eq) and Na₂CO₃ (60.9 mg, 575 umol, 3 eq) in one portion at 20° C. under N₂ protection. The mixture was stirred at 75° C. and stirred for 6 hrs. After cooling to 20° C., the reaction mixture was concentrated to give a residue, which was purified by silica gel chromatography (petroleum ether/ethyl acetate=1/1) to afford tert-butyl 4-[6-[2-methyl-8-(2-oxo-1-pyridyl)imidazo[1,2-b]pyridazin-6-yl]-1-oxo-2-isoquinolyl]piperidine-1-carboxylate (50.0 mg, 47%) as a solid. LCMS: (ESI, m/z): 553.1 [M+H]⁺. ¹H NMR (400 MHz, MeOD-d₄) δ ppm 8.42 (d, J=8.31 Hz, 1H) 8.29 (s, 1H) 8.19 (br d, J=8.31 Hz, 1H) 8.09 (d, J=16.63 Hz, 2H) 7.68-7.81 (m, 2H) 7.50 (d, J=7.83 Hz, 1H) 6.71-6.82 (m, 2H) 6.59-6.59 (m, 1H) 6.59 (t, J=6.60 Hz, 1H) 5.02-5.15 (m, 1H) 4.31 (br d, J=12.72 Hz, 2H) 2.88-3.09 (m, 2H) 2.47 (s, 3H) 1.85-1.97 (m, 4H) 1.49 (s, 9H)

### Synthesis of Compound 552

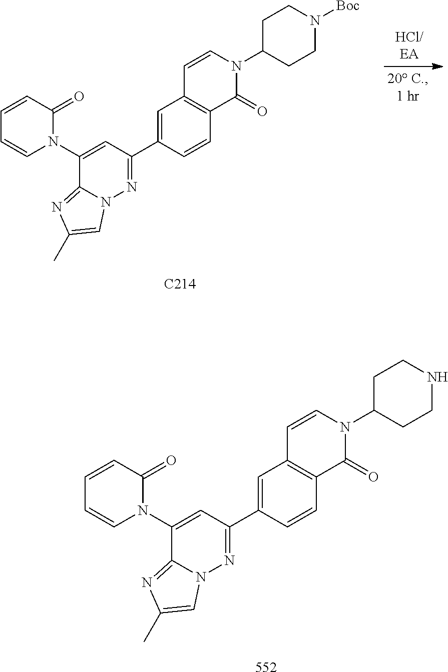 [SMILES: Cc1cn2nc(-c3ccc4c(=O)n(C5CCN(C(=O)OC(C)(C)C)CC5)ccc4c3)cc(-n3ccccc3=O)c2n1 ; "C214" ; Cc1cn2nc(-c3ccc4c(=O)n(C5CCNCC5)ccc4c3)cc(-n3ccccc3=O)c2n1 ; "552"]

To a mixture of tert-butyl 4-[6-[2-methyl-8-(2-oxo-1-pyridyl)imidazo[1,2-b]pyridazin-6-yl]-1-oxo-2-isoquinolyl]piperidine-1-carboxylate (50.0 mg, 90.4 umol, 1 eq) in ethyl acetate (500 uL) was added HCl/ethyl acetate (2 M, 500 uL) at 20° C. The mixture was stirred at 20° C. for 1 hr. The mixture was concentrated under reduced pressure at 45° C. The crude product was triturated with dichloromethane (5.00 mL) to afford 6-[2-methyl-8-(2-oxo-1-pyridyl)imidazo[1,2-b]pyridazin-6-yl]-2-(4-piperidyl)isoquinolin-1-one (16.4 mg, 40%) as a solid. LCMS: (ESI, m/z): 453.1 [M+H]⁺. ¹H NMR (400 MHz, MeOD-d₄) δ ppm 8.66 (s, 1H) 8.48-8.57 (m, 3H) 8.34 (dd, J=8.56, 1.71 Hz, 1H) 7.77-7.87 (m, 2H) 7.51 (d, J=7.34 Hz, 1H) 6.93 (d, J=7.82 Hz, 1H) 6.80 (d, J=9.29 Hz, 1H) 6.65-6.72 (m, 1H) 5.15 (s, 1H) 3.63 (br d, J=13.20 Hz, 2H) 3.27 (br s, 2H) 2.64 (s, 3H) 2.35 (br dd, J=12.96, 3.67 Hz, 2H) 2.19 (br d, J=13.20 Hz, 2H)

### Chromatography:

### Prep-HPLC

Condition G1: Column: XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient 1: 5% B to 45% B in 8 min; Gradient 2: 5% B to 30% B in 8 min; Gradient 3: 5% B to 55% B in 8 min; Gradient 4: 10% B to 60% B in 10 min; Gradient 5: 30% B to 80% B in 15 min; Gradient 6: 35% B to 65% B in 8 min; Gradient 7: 25% B to 55% B in 10 min; Gradient 8: 60% B to 90% B in 8 min; Gradient 9: 70% B to 95% B in 8 min; Gradient 10: 50% B to 70% B in 8 min.
Condition G2: Column: YMC-Actus Triart C18, 30*150 mm, 5 um; Mobile Phase A: Water (10 mmol/L NH₄HCO₃), Mobile Phase B: acetonitrile; Flow rate: 60 mL/min; Gradient 1: 5% B to 50% B in 8 min; Gradient 2: 5% B to 40% B in 10 min.
Condition G3: Column: Xselect CSH OBD Column 30*150 mm 5 um, n; Mobile Phase A: Water (10 mmol/L NH₄HCO₃), Mobile Phase B: acetonitrile; Flow rate: 60 mL/min; Gradient 1: 5% B to 65% B in 8 min.
Condition G4: column, C18 silica gel, XBridge, 19×150 mm; mobile phase, Mobile Phase A: water (0.05% NH₃H₂O), Mobile Phase B: acetonitrile; Gradient 1: 25% B to 60% B in 8 min; Gradient 2: 20% B to 70% B in 12 min; Gradient 3: 40% B to 65% B in 12 min.
Condition G5: column, C18 silica gel, XBridge, 19×150 mm; Mobile Phase A: water (0.05% NH₄HCO₃), Mobile Phase B: acetonitrile; Gradient 1: 20% B to 50% B in 15 min.
Condition G6: column, C18 silica gel; mobile phase A: water (0.1% TFA), mobile phase B: acetonitrile; Gradient 1: 10% B to 50% B in 10 min.
Condition G7: column, C18 silica gel; mobile phase A, water (0.1% NH₃·H₂O), mobile phase B: acetonitrile; Gradient 1: 10% B to 50% B in 10 min.
Condition G8: column: Phenomenex Luna 80*30 mm*3 um; mobile phase A: water (HCl), mobile phase B: acetonitrile; Gradient 1: B %: 1%-30%, 8 min, Gradient 2: B %: 1%-40%, 8 min; Gradient 3: 30% B to 65% B in 7 min; Gradient 4: 20% B to 50% B in 8 min; Gradient 5:B %: 10%-40%, 8 min; Gradient 6: 5% to 30% B in 8 min.
Condition G9: column: Welch Xtimate C18 250×70 mm #10 um; mobile phase A: water (NH₄HCO₃); mobile phase B: acetonitrile; Gradient 1: B %: 35%-65%, 20 min.
Condition G10: column: Phenomenex Luna C18 75*30 mm*3 um; mobile phase A: water (TFA); mobile phase B: ACN; Gradient 1: B %: 15%-45%, 8 min.

### Reverse Flash

Condition G1: Column, silica gel; Mobile Phase A: water; Mobile Phase B: acetonitrile, Gradient 1: 10% B to 50% B in 10 min.
Condition G2: column, C18 silica gel; mobile phase A: water (0.1% NH₃·H₂O), mobile phase B: acetonitrile; Gradient 1: 20% B to 50% B in 10 min.
Condition G3: column, C18 silica gel; mobile phase A: water (0.1% HCl), mobile phase B: acetonitrile; Gradient 1: 0% B to 100% B in 10 min.
Condition G4: column, C18 silica gel; mobile phase A: water (0.1% formic acid), mobile phase B: acetonitrile; Gradient 1: 10% B to 85% B in 10 min; Gradient 2: 5% B to 80% B.
Condition G5: Gemini® 5 μm NX-C18 110 A, 100×30 mm column; mobile phase A: 10 mM aqueous ammonium formate; mobile phase B: acetonitrile; Gradient 1: 25% B to 100% B.
Condition G6: ChiralPak IC column (length: 150 mm, diameter: 4.6 mm), mobile phase A: EtOH with 0.1% NH₄OH in CO₂, mobile phase B: acetonitrile; Gradient 1: of 5% B-60% B.

### Chiral HPLC

Condition G1: Column: CHIRALPAK IG-3, 4.6*50 mm 3 um; Mobile Phase A: MtBE (0.1% DEA):MeOH=50: 50; Flow rate: 1 mL/min; Gradient: 0% B to 0% B.

### SFC

Condition G1: column: REGIS (s,s) WHELK-O1 (250 mm*50 mm, 10 um); mobile phase: [0.1% NH₃H₂O IPA]; Gradient 1: B % 55%-55%, 10 min.
Condition G2: column: Chiral Pak IH, 250*30 mm, 10 um; mobile phase: [0.1% NH₃H₂O ethanol]; B %: 45%-45%, 7 min
Condition G3: column: DAICEL CHIRALCEL OZ 250×25 mm I.D. 10 um; mobile phase: [0.1% NH₃H₂O methanol]; B %: 60%-60%, 15 min.
Condition G4: column: ChiralPak IH, 250*30 mm, 10 um; mobile phase: [Neu-IPA]; Gradient: 40%-40%, 12 min
Condition G5: column: REGIS (S,S) WHELK-O1(250 mm×25 mm, 10 um); mobile phase: [Neu-CH₃OH]; B %: 75%-75%, 15 min

### Example G1: Synthesis of Compound 215

### Synthesis of Intermediate G1

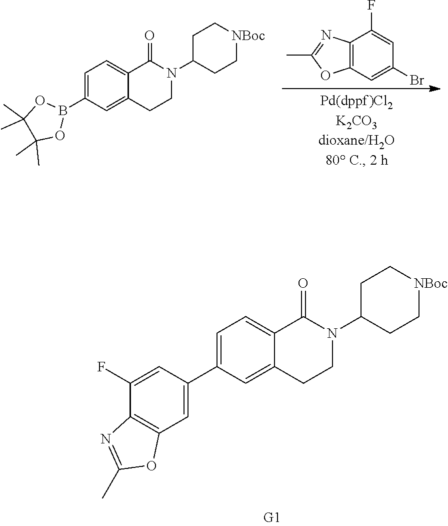

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (120.00 mg, 0.263 mmol, 1.00 equiv) and 6-bromo-4-fluoro-2-methyl-1,3-benzoxazole (66.53 mg, 0.289 mmol, 1.10 equiv) in dioxane (2.00 mL) and water (0.50 mL) were added Pd(dppf)Cl₂ CH₂Cl₂(10.71 mg, 0.013 mmol, 0.05 equiv) and K₂CO₃ (109.02 mg, 0.789 mmol, 3.00 equiv). The reaction mixture was stirred for 2 h at 80° C. under a nitrogen atmosphere, then concentrated under reduced pressure to give a residue. The residue was purified by silica gel column chromatography, eluted with CH₂Cl₂/MeOH (15:1) to afford tert-butyl 4-[6-(4-fluoro-2-methyl-1,3-benzoxazol-6-yl)-1-oxo-3,4-dihydroisoquinolin-2-yl]piperidine-1-carboxylate (100 mg, 79%) as a solid. LCMS (ES, m/z): 480 [M+H]⁺.

### Synthesis of Compound 215

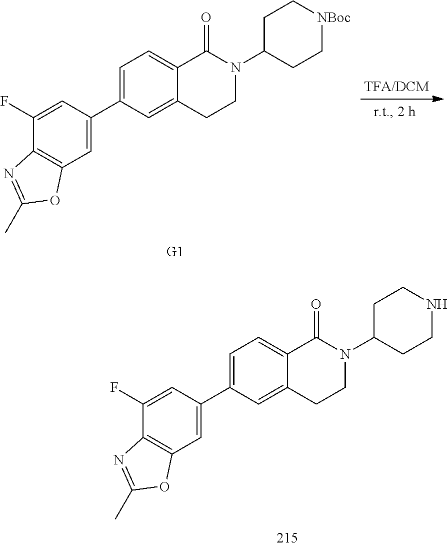 [SMILES: Cc1nc2c(F)cc(-c3ccc4c(c3)CCN(C3CCN(C(=O)OC(C)(C)C)CC3)C4=O)cc2o1 ; Cc1nc2c(F)cc(-c3ccc4c(c3)CCN(C3CCNCC3)C4=O)cc2o1]

A mixture of tert-butyl 4-[6-(4-fluoro-2-methyl-1,3-benzoxazol-6-yl)-1-oxo-3,4-dihydroisoquinolin-2-yl]piperidine-1-carboxylate (100 mg, 1 equiv) and TFA (0.20 mL) in DCM (1.00 mL) was stirred for 2 h at room temperature under nitrogen atmosphere. The resulting mixture was concentrated under reduced pressure to give a residue. The residue was purified by Prep-HPLC (Condition G1, Gradient 1) to afford 6-(4-fluoro-2-methyl-3a,7a-dihydro-1,3-benzoxazol-6-yl)-2-(piperidin-4-yl)-3,4-dihydroisoquinolin-1-one (24.2 mg, 30%) as a solid. LCMS (ES, m/z): 380 [M+H]⁺. ¹H NMR (400 MHz, DMSO-d6) δ 7.98-7.92 (m, 2H), 7.79-7.70 (m, 2H), 7.64 (dd, J=11.5, 1.4 Hz, 1H), 4.54 (ddt, J=12.0, 8.0, 4.1 Hz, 1H), 3.48 (dd, J=8.5, 4.5 Hz, 2H), 3.01 (q, J=7.8, 6.6 Hz, 4H), 2.67 (s, 3H), 2.57 (dd, J=12.1, 2.6 Hz, 2H), 1.62 (qd, J=12.0, 4.1 Hz, 2H), 1.51 (d, J=10.1 Hz, 2H).

### Example G2: Synthesis of Compound 216

### Synthesis of Intermediate G2

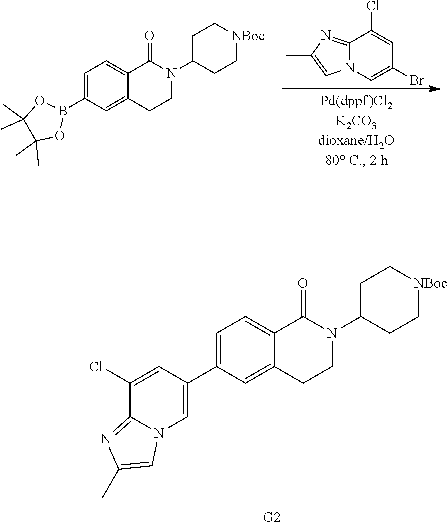 [SMILES: CC(C)(C)OC(=O)N1CCC(N2CCc3cc(B4OC(C)(C)C(C)(C)O4)ccc3C2=O)CC1 ; Cc1cn2cc(Br)cc(Cl)c2n1 ; Cc1cn2cc(-c3ccc4c(c3)CCN(C3CCN(C(=O)OC(C)(C)C)CC3)C4=O)cc(Cl)c2n1]

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (150.00 mg, 0.329 mmol, 1.00 equiv) and 6-bromo-8-chloro-2-methylimidazo[1,2-a]pyridine (88.76 mg, 0.362 mmol, 1.10 equiv) in dioxane (2 mL) and water (0.5 mL) were added Pd(dppf)Cl₂ CH₂Cl₂ (13.39 mg, 0.016 mmol, 0.05 equiv) and K₂CO₃ (136.27 mg, 0.987 mmol, 3.00 equiv). The reaction mixture was stirred for 2 h at 80° C. under a nitrogen atmosphere, then concentrated under reduced pressure to give a residue. The residue was purified by silica gel column chromatography, eluted with CH₂Cl₂/MeOH (15:1) to afford tert-butyl 4-(6-[8-chloro-2-methylimidazo[1,2-a]pyridin-6-yl]-1-oxo-3,4-dihydroisoquinolin-2-yl) piperidine-1-carboxylate (110 mg, 68%) as a solid. LCMS (ES, m/z): 495 [M+H]⁺.

### Synthesis of Compound 216

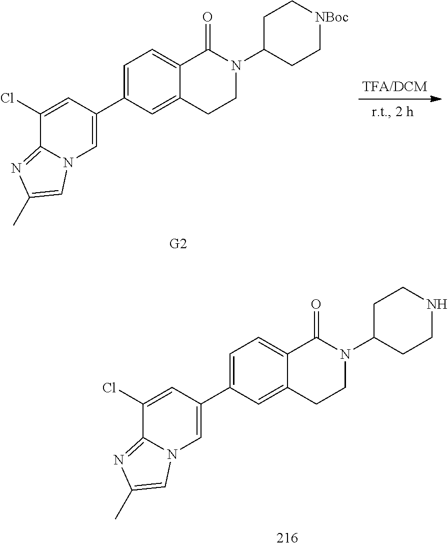 [SMILES: Cc1cn2cc(-c3ccc4c(c3)CCN(C3CCN(C(=O)OC(C)(C)C)CC3)C4=O)cc(Cl)c2n1 ; Cc1cn2cc(-c3ccc4c(c3)CCN(C3CCNCC3)C4=O)cc(Cl)c2n1]

A mixture of tert-butyl 4-(6-[8-chloro-2-methylimidazo[1,2-a]pyridin-6-yl]-1-oxo-3,4-dihydroisoquinolin-2-yl) piperidine-1-carboxylate (100.00 mg, 0.202 mmol, 1.00 equiv) and TFA (0.20 mL) in DCM (1.00 mL) was stirred for 2 h at room temperature under nitrogen atmosphere. The resulting mixture was concentrated under reduced pressure to give a residue. The residue was purified by Prep-HPLC (Condition G1, Gradient 2) to afford 6-[8-chloro-2-methylimidazo[1,2-a]pyridin-6-yl]-2-(piperidin-4-yl)-3,4-dihydroisoquinolin-1-one (26.2 mg, 33%) as a solid. LCMS (ES, m/z): 395 [M+H]⁺. ¹H NMR (400 MHz, DMSO-d6) δ 8.96 (d, J=1.6 Hz, 1H), 7.95 (d, J=8.0 Hz, 1H), 7.82 (dd, J=11.1, 1.4 Hz, 2H), 7.74-7.67 (m, 2H), 4.59-4.49 (m, 1H), 3.48 (t, J=6.5 Hz, 2H), 3.06-2.95 (m, 4H), 2.57 (dd, J=12.1, 2.6 Hz, 2H), 2.39 (d, J=0.9 Hz, 3H), 1.62 (qd, J=12.0, 4.1 Hz, 2H), 1.51 (d, J=11.0 Hz, 2H).

### Example G3: Synthesis of Compound 217

### Synthesis of Intermediate G3

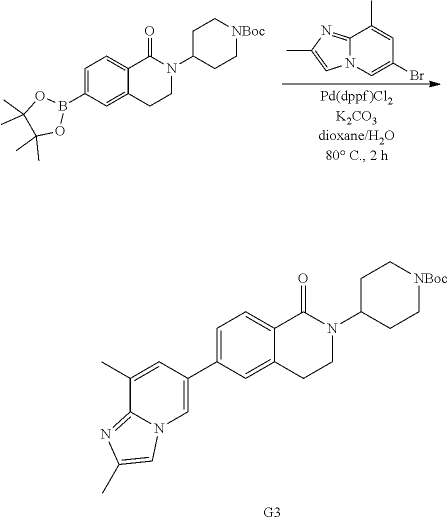 [SMILES: CC(C)(C)OC(=O)N1CCC(N2CCc3cc(B4OC(C)(C)C(C)(C)O4)ccc3C2=O)CC1 ; Cc1cn2cc(Br)cc(C)c2n1 ; Cc1cn2cc(-c3ccc4c(c3)CCN(C3CCN(C(=O)OC(C)(C)C)CC3)C4=O)cc(C)c2n1]

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (150.00 mg, 0.329 mmol, 1.00 equiv) and 6-bromo-2,8-dimethylimidazo[1,2-a]pyridine (81.38 mg, 0.362 mmol, 1.10 equiv) in dioxane (2 mL) and water (0.5 mL) were added Pd(dppf)Cl₂ CH₂Cl₂(13.39 mg, 0.016 mmol, 0.05 equiv) and K₂CO₃ (136.27 mg, 0.986 mmol, 3.00 equiv). The reaction mixture was stirred for 2 h at 80° C. under a nitrogen atmosphere, then concentrated under reduced pressure to give a residue. The residue was purified by silica gel column chromatography, eluted with CH₂Cl₂/MeOH (15:1) to afford tert-butyl 4-(6-[2,8-dimethylimidazo[1,2-a]pyridin-6-yl]-1-oxo-3,4-dihydroisoquinolin-2-yl) piperidine-1-carboxylate (120 mg, 77%) as a solid. LCMS (ES, m/z): 475 [M+H]⁺.

### Synthesis of Compound 217

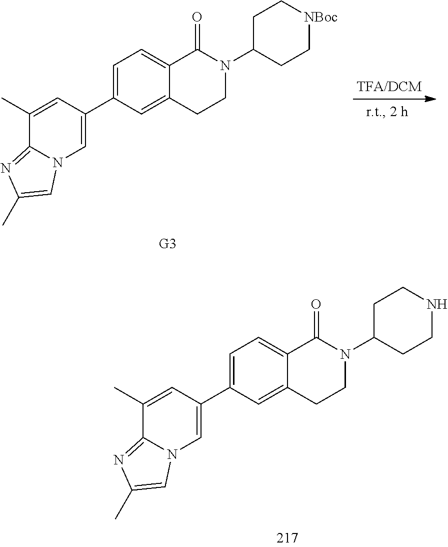 [SMILES: Cc1cn2cc(-c3ccc4c(c3)CCN(C3CCN(C(=O)OC(C)(C)C)CC3)C4=O)cc(C)c2n1 ; Cc1cn2cc(-c3ccc4c(c3)CCN(C3CCNCC3)C4=O)cc(C)c2n1]

A mixture of tert-butyl 4-(6-[2,8-dimethylimidazo[1,2-a]pyridin-6-yl]-1-oxo-3,4-dihydroisoquinolin-2-yl) piperidine-1-carboxylate (100.00 mg, 0.211 mmol, 1.00 equiv) and TFA (0.20 mL) in DCM (1.00 mL) was stirred for 2 h at room temperature under nitrogen atmosphere. The resulting mixture was concentrated under reduced pressure to give a residue. The residue was purified by Prep-HPLC (Condition G1, Gradient 2) to afford 6-[2,8-dimethylimidazo[1,2-a]pyridin-6-yl]-2-(piperidin-4-yl)-3,4-dihydroisoquinolin-1-one (36.4 mg, 46%) as a solid. LCMS (ES, m/z): 375 [M+H]⁺. ¹H NMR (400 MHz, DMSO-d6) δ 8.77 (d, J=2.0 Hz, 1H), 7.94 (d, J=8.0 Hz, 1H), 7.71-7.62 (m, 3H), 7.41 (t, J=1.6 Hz, 1H), 4.54 (m, 1H), 3.48 (t, J=6.4 Hz, 2H), 3.07-2.91 (m, 4H), 2.57 (d, J=11.9 Hz, 2H), 2.52 (s, 3H), 2.36 (d, J=0.9 Hz, 3H), 1.68-1.56 (m, 2H), 1.51 (d, J=11.9 Hz, 2H).

### Example G4: Synthesis of Compound 218

### Synthesis of Intermediate G4

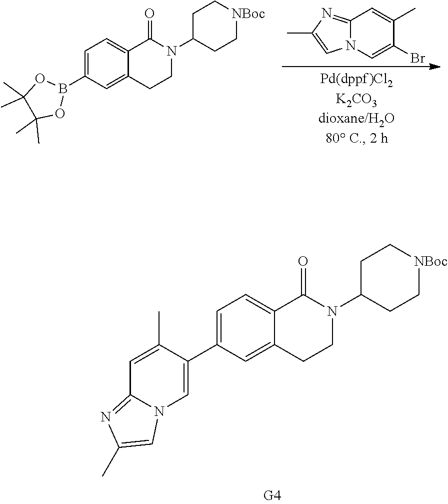

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (150.00 mg, 0.329 mmol, 1.00 equiv) and 6-bromo-2,7-dimethylimidazo[1,2-a]pyridine (81.38 mg, 0.362 mmol, 1.10 equiv) in dioxane (2.00 mL) and water (0.50 mL) were added Pd(dppf)Cl₂ CH₂Cl₂ (13.39 mg, 0.016 mmol, 0.05 equiv) and K₂CO₃ (136.27 mg, 0.987 mmol, 3.00 equiv). The reaction mixture was stirred for 2 h at 80° C. under a nitrogen atmosphere, then concentrated under reduced pressure to give a residue. The residue was purified by silica gel column chromatography, eluted with CH₂Cl₂/MeOH (15:1) to afford tert-butyl 4-(6-[2,7-dimethylimidazo[1,2-a]pyridin-6-yl]-1-oxo-3,4-dihydroisoquinolin-2-yl) piperidine-1-carboxylate (120 mg, 77%) as a solid. LCMS (ES, m/z): 475 [M+H]⁺.

### Synthesis of Compound 218

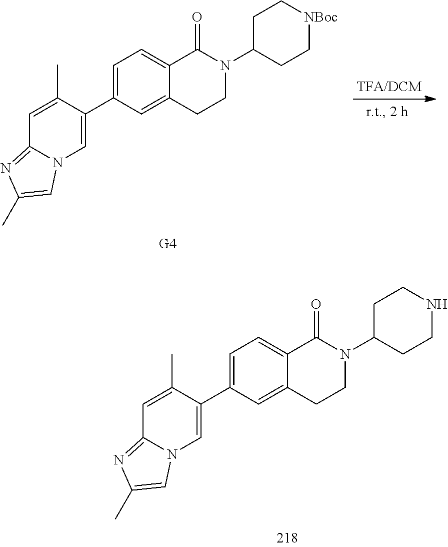 [SMILES: Cc1cn2cc(-c3ccc4c(c3)CCN(C3CCN(C(=O)OC(C)(C)C)CC3)C4=O)c(C)cc2n1 ; Cc1cn2cc(-c3ccc4c(c3)CCN(C3CCNCC3)C4=O)c(C)cc2n1]

A mixture of tert-butyl 4-(6-[2,7-dimethylimidazo[1,2-a]pyridin-6-yl]-1-oxo-3,4-dihydroisoquinolin-2-yl) piperidine-1-carboxylate (100.00 mg, 0.211 mmol, 1.00 equiv) and TFA (0.20 mL) in DCM (1.00 mL) was stirred for 2 h at room temperature under nitrogen atmosphere. The resulting mixture was concentrated under reduced pressure to give a residue. The residue was purified by Prep-HPLC (Condition G1, Gradient 1) to afford 6-[2,7-dimethylimidazo[1,2-a]pyridin-6-yl]-2-(piperidin-4-yl)-3,4-dihydroisoquinolin-1-one (39.7 mg, 50%) as a solid. LCMS (ES, m/z): 375 [M+H]⁺. ¹H NMR (400 MHz, DMSO-d6) δ 8.36 (s, 1H), 7.94 (d, J=7.9 Hz, 1H), 7.59 (s, 1H), 7.42-7.33 (m, 3H), 4.62-4.51 (m, 1H), 3.49 (t, J=6.4 Hz, 2H), 3.05 (d, J=12.5 Hz, 2H), 2.98 (t, J=6.4 Hz, 2H), 2.60 (td, J=12.2, 2.6 Hz, 2H), 2.32 (d, J=0.9 Hz, 3H), 2.23 (d, J=1.1 Hz, 3H), 1.66 (qd, J=12.0, 4.1 Hz, 2H), 1.54 (d, J=11.5 Hz, 2H).

### Example G5: Synthesis of Compound 219

### Synthesis of Intermediate G5

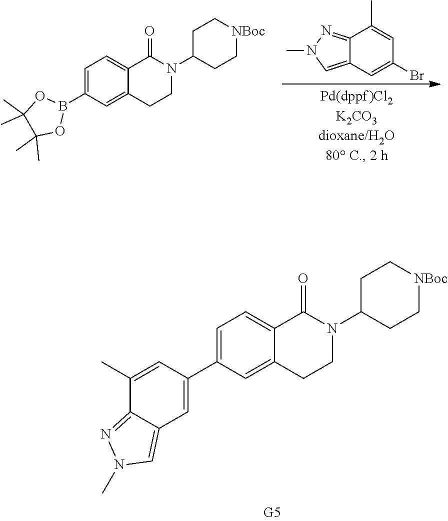 [SMILES: CC(C)(C)OC(=O)N1CCC(N2CCc3cc(B4OC(C)(C)C(C)(C)O4)ccc3C2=O)CC1 ; Cc1cc(Br)cc2cn(C)nc12 ; Cc1cc(-c2ccc3c(c2)CCN(C2CCN(C(=O)OC(C)(C)C)CC2)C3=O)cc2cn(C)nc12]

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (150.00 mg, 0.329 mmol, 1.00 equiv) and 5-bromo-2,7-dimethylindazole (81.38 mg, 0.362 mmol, 1.10 equiv) in dioxane (2.00 mL) and water (0.50 mL) were added Pd(dppf)Cl₂ CH₂Cl₂ (13.39 mg, 0.016 mmol, 0.05 equiv) and K₂CO₃ (136.27 mg, 0.986 mmol, 3.00 equiv). The reaction mixture was stirred for 2 h at 80° C. under a nitrogen atmosphere, then concentrated under reduced pressure to give a residue. The residue was purified by silica gel column chromatography, eluted with CH₂Cl₂/MeOH (15:1) to afford tert-butyl 4-[6-(2,7-dimethylindazol-5-yl)-1-oxo-3,4-dihydroisoquinolin-2-yl]piperidine-1-carboxylate (120 mg, 77%) as a solid. LCMS (ES, m/z): 475 [M+H]⁺.

### Synthesis of Compound 219

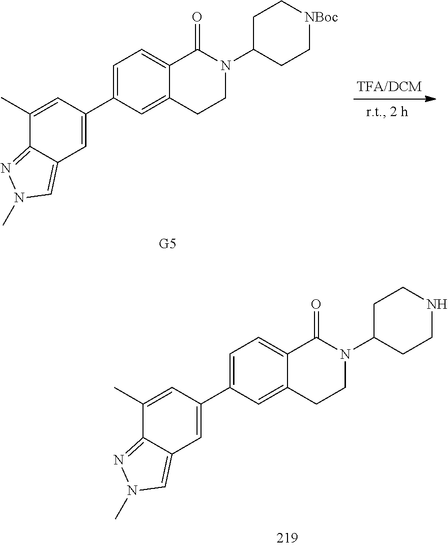 [SMILES: Cc1cc(-c2ccc3c(c2)CCN(C2CCN(C(=O)OC(C)(C)C)CC2)C3=O)cc2cn(C)nc12 ; Cc1cc(-c2ccc3c(c2)CCN(C2CCNCC2)C3=O)cc2cn(C)nc12]

A mixture of tert-butyl 4-[6-(2,7-dimethylindazol-5-yl)-1-oxo-3,4-dihydroisoquinolin-2-yl]piperidine-1-carboxylate (100.00 mg, 0.211 mmol, 1.00 equiv) and TFA (0.20 mL) in DCM (1.00 mL) was stirred for 2 h at room temperature under nitrogen atmosphere. The resulting mixture was concentrated under reduced pressure to give a residue. The residue was purified by Prep-HPLC (Condition G1, Gradient 1) to give 6-(2,7-dimethylindazol-5-yl)-2-(piperidin-4-yl)-3,4-dihydroisoquinolin-1-one (37.7 mg, 48%) as a solid. LCMS (ES, m/z): 375 [M+H]⁺. ¹H NMR (400 MHz, DMSO-d6) δ 8.38 (s, 1H), 7.92 (d, J=8.1 Hz, 1H), 7.89-7.84 (m, 1H), 7.70-7.60 (m, 2H), 7.40 (t, J=1.4 Hz, 1H), 4.60-4.49 (m, 1H), 4.20 (s, 3H), 3.48 (t, J=6.5 Hz, 2H), 3.06-2.95 (m, 4H), 2.57 (t, J=0.7 Hz, 5H), 1.62 (qd, J=11.8, 4.0 Hz, 2H), 1.51 (d, J=11.2 Hz, 2H).

### Example G6: Synthesis of Compound 220

### Synthesis of Intermediate G6

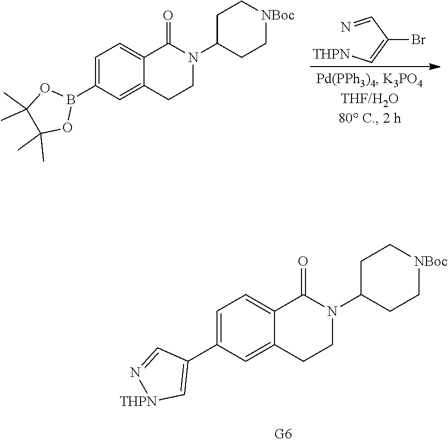

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (150.00 mg, 0.329 mmol, 1.00 equiv) and 4-bromo-1-(oxan-2-yl) pyrazole (83.55 mg, 0.362 mmol, 1.10 equiv) in THF (2.00 mL) and water (1.00 mL) were added Pd(PPh₃)₄(18.99 mg, 0.016 mmol, 0.05 equiv) and K₃PO₄ (209.29 mg, 0.986 mmol, 3.00 equiv). The reaction mixture was stirred for 2 h at 80° C. under a nitrogen atmosphere, then concentrated under reduced pressure to give a residue. The residue was purified by silica gel column chromatography, eluted with CH₂Cl₂/MeOH (15:1) to afford tert-butyl 4-[6-[1-(oxan-2-yl) pyrazol-4-yl]-1-oxo-3,4-dihydroisoquinolin-2-yl]piperidine-1-carboxylate (120 mg, 76%) as an oil. LCMS (ES, m/z): 481 [M+H]⁺.

### Synthesis of Compound 220

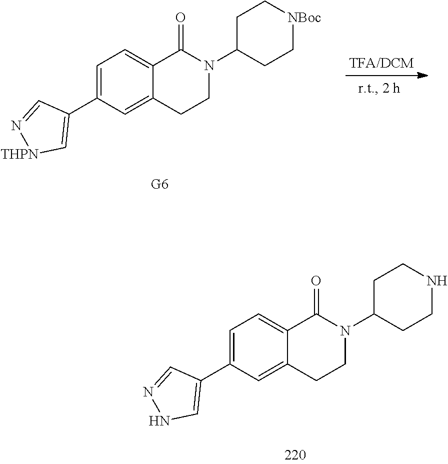

A mixture of tert-butyl 4-[6-[1-(oxan-2-yl) pyrazol-4-yl]-1-oxo-3,4-dihydroisoquinolin-2-yl]piperidine-1-carboxylate (120.00 mg, 0.250 mmol, 1.00 equiv) and TFA (0.20 mL) in DCM (1.00 mL) was stirred for 2 h at room temperature under nitrogen atmosphere. The resulting mixture was concentrated under reduced pressure to give a residue. The residue was purified by Prep-HPLC (Condition G1, Gradient 2) to afford 2-(piperidin-4-yl)-6-(1H-pyrazol-4-yl)-3,4-dihydroisoquinolin-1-one (40.7 mg, 55%) as a solid. LCMS (ES, m/z): 297 [M+H]⁺. ¹H NMR (400 MHz, DMSO-d6) δ 13.01 (s, 1H), 8.14 (s, 2H), 7.83 (d, J=8.1 Hz, 1H), 7.59 (dd, J=8.0, 1.8 Hz, 1H), 7.54 (d, J=1.7 Hz, 1H), 4.62-4.51 (m, 1H), 3.44 (t, J=6.5 Hz, 2H), 3.09 (d, J=12.6 Hz, 2H), 2.93 (t, J=6.4 Hz, 2H), 2.65 (td, J=12.3, 2.8 Hz, 2H), 1.67 (qd, J=12.3, 4.1 Hz, 2H), 1.59-1.51 (m, 2H).

### Example G7: Synthesis of Compound 221

### Synthesis of Intermediate G7

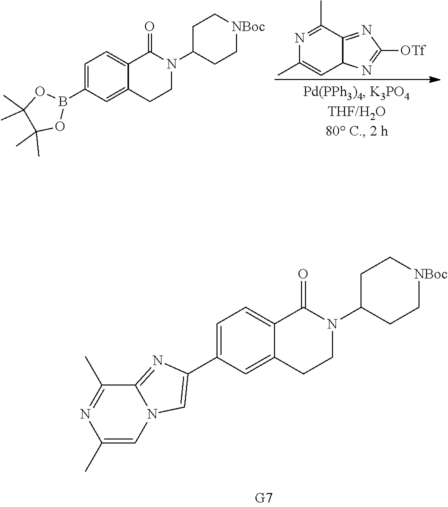 [SMILES: CC(C)(C)OC(=O)N1CCC(N2CCc3cc(B4OC(C)(C)C(C)(C)O4)ccc3C2=O)CC1 ; CC1=CC2N=C(OS(=O)(=O)C(F)(F)F)N=C2C(C)=N1 ; Cc1cn2cc(-c3ccc4c(c3)CCN(C3CCN(C(=O)OC(C)(C)C)CC3)C4=O)nc2c(C)n1]

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (150.00 mg, 0.329 mmol, 1.00 equiv) and 6,8-dimethylimidazo[1,2-a]pyrazin-2-yl trifluoromethanesulfonate (106.74 mg, 0.362 mmol, 1.10 equiv) in THF (2.00 mL) and water (1.00 mL) were added Pd(PPh₃)₄(18.99 mg, 0.016 mmol, 0.05 equiv) and K₃PO₄ (209.29 mg, 0.986 mmol, 3.00 equiv). The reaction mixture was stirred for 2 h at 80° C. under a nitrogen atmosphere, then concentrated under reduced pressure to give a residue. The residue was purified by silica gel column chromatography, eluted with CH₂Cl₂/MeOH (15:1) to afford tert-butyl 4-(6-[6,8-dimethylimidazo[1,2-a]pyrazin-2-yl]-1-oxo-3,4-dihydroisoquinolin-2-yl) piperidine-1-carboxylate (120 mg, 77%) as an oil. LCMS (ES, m/z): 476 [M+H]⁺.

### Synthesis of Compound 221

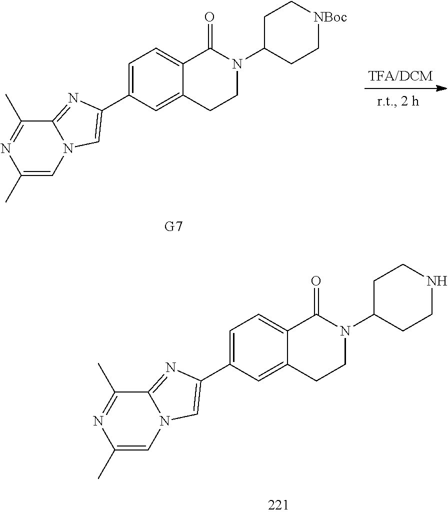 [SMILES: Cc1cn2cc(-c3ccc4c(c3)CCN(C3CCN(C(=O)OC(C)(C)C)CC3)C4=O)nc2c(C)n1 ; Cc1cn2cc(-c3ccc4c(c3)CCN(C3CCNCC3)C4=O)nc2c(C)n1]

A mixture of tert-butyl 4-(6-[6,8-dimethylimidazo[1,2-a]pyrazin-2-yl]-1-oxo-3,4-dihydroisoquinolin-2-yl) piperidine-1-carboxylate (100.00 mg, 0.210 mmol, 1.00 equiv) and TFA (0.20 mL) in DCM (1.00 mL) was stirred for 2 h at room temperature under nitrogen atmosphere. The resulting mixture was concentrated under reduced pressure to give a residue. The residue was purified by Prep-HPLC (Condition G1, Gradient 1) to give 6-[6,8-dimethylimidazo[1,2-a]pyrazin-2-yl]-2-(piperidin-4-yl)-3,4-dihydroisoquinolin-1-one (33.9 mg, 43%) as a solid. LCMS (ES, m/z): 376 [M+H]⁺. ¹H NMR (400 MHz, DMSO-d6) δ 8.54 (s, 1H), 8.27 (s, 1H), 8.01-7.91 (m, 3H), 4.57 (ddt, J=12.1, 8.0, 4.1 Hz, 1H), 3.48 (t, J=6.5 Hz, 2H), 3.09-2.97 (m, 4H), 2.76 (s, 3H), 2.60 (dd, J=12.9, 10.4 Hz, 2H), 2.39 (d, J=1.0 Hz, 3H), 1.65 (qd, J=12.0, 4.1 Hz, 2H), 1.53 (d, J=10.9 Hz, 2H).

### Example G8: Synthesis of Compound 222

### Synthesis of Intermediate G8

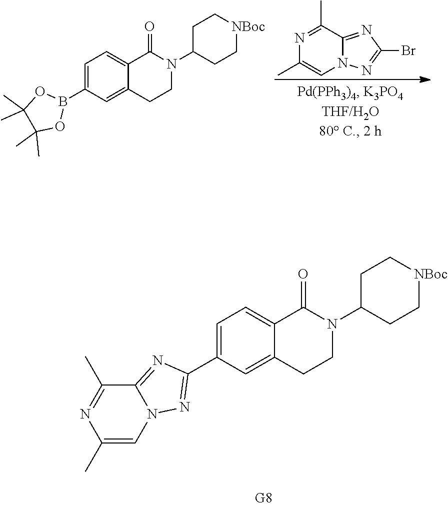 [SMILES: CC(C)(C)OC(=O)N1CCC(N2CCc3cc(B4OC(C)(C)C(C)(C)O4)ccc3C2=O)CC1 ; Cc1cn2nc(Br)nc2c(C)n1 ; Cc1cn2nc(-c3ccc4c(c3)CCN(C3CCN(C(=O)OC(C)(C)C)CC3)C4=O)nc2c(C)n1]

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (150.00 mg, 0.329 mmol, 1.00 equiv) and 2-bromo-6,8-dimethyl-[1,2,4]triazolo[1,5-a]pyrazine (82.09 mg, 0.362 mmol, 1.10 equiv) in THE (2.00 mL) and water (1.00 mL) were added Pd(PPh₃)₄(18.99 mg, 0.016 mmol, 0.05 equiv) and K₃PO₄ (209.29 mg, 0.987 mmol, 3.00 equiv). The reaction mixture was stirred for 2 h at 80° C. under a nitrogen atmosphere, then concentrated under reduced pressure to give a residue. The residue was purified by silica gel column chromatography, eluted with CH₂Cl₂/MeOH (15:1) to afford tert-butyl 4-(6-[6,8-dimethyl-[1,2,4]triazolo[1,5-a]pyrazin-2-yl]-1-oxo-3,4-dihydroisoquinolin-2-yl) piperidine-1-carboxylate (110 mg, 70%) as an oil. LCMS (ES, m/z): 477 [M+H]⁺.

### Synthesis of Compound 222

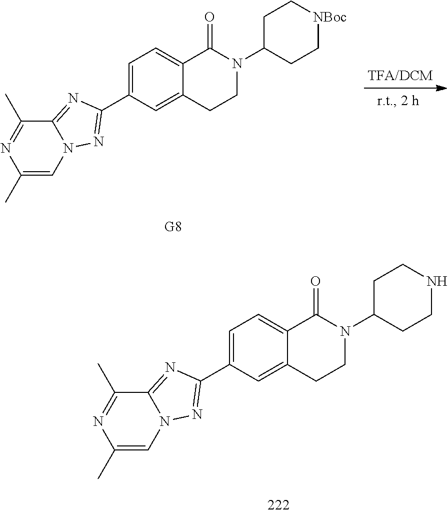 [SMILES: Cc1cn2nc(-c3ccc4c(c3)CCN(C3CCN(C(=O)OC(C)(C)C)CC3)C4=O)nc2c(C)n1 ; Cc1cn2nc(-c3ccc4c(c3)CCN(C3CCNCC3)C4=O)nc2c(C)n1]

A mixture of tert-butyl 4-(6-[6,8-dimethyl-[1,2,4]triazolo[1,5-a]pyrazin-2-yl]-1-oxo-3,4-dihydroisoquinolin-2-yl) piperidine-1-carboxylate (100 mg, 1 equiv) and TFA (0.20 mL) in DCM (1.00 mL) was stirred for 2 h at room temperature under nitrogen atmosphere. The resulting mixture was concentrated under reduced pressure to give a residue. The residue was purified by Prep-HPLC (Condition G1, Gradient 3) to afford 6-[6,8-dimethyl-[1,2,4]triazolo[1,5-a]pyrazin-2-yl]-2-(piperidin-4-yl)-3,4-dihydroisoquinolin-1-one (36.2 mg, 46%) as a solid. LCMS (ES, m/z): 377 [M+H]⁺. ¹H NMR (400 MHz, DMSO-d6) δ 8.80 (s, 1H), 8.18 (dd, J=8.0, 1.7 Hz, 1H), 8.13 (d, J=1.7 Hz, 1H), 8.04 (d, J=8.1 Hz, 1H), 4.55 (ddt, J=11.9, 8.1, 4.1 Hz, 1H), 3.50 (t, J=6.5 Hz, 2H), 3.05 (q, J=6.0 Hz, 4H), 2.84 (s, 3H), 2.51 (s, 3H), 2.49 (s, 3H), 1.65 (qd, J=12.2, 4.1 Hz, 2H), 1.57-1.48 (m, 2H).

### Example G9: Synthesis of Compound 223

### Synthesis of Intermediate G9

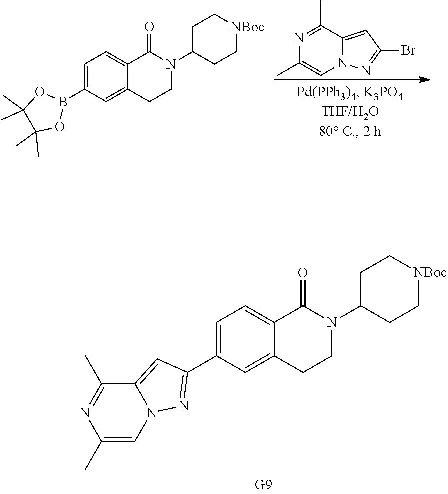 [SMILES: CC(C)(C)OC(=O)N1CCC(N2CCc3cc(B4OC(C)(C)C(C)(C)O4)ccc3C2=O)CC1 ; Cc1cn2nc(Br)cc2c(C)n1 ; Cc1cn2nc(-c3ccc4c(c3)CCN(C3CCN(C(=O)OC(C)(C)C)CC3)C4=O)cc2c(C)n1]

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (150.00 mg, 0.329 mmol, 1.00 equiv) and 2-bromo-4,6-dimethylpyrazolo[1,5-a]pyrazine (81.73 mg, 0.362 mmol, 1.10 equiv) in THF (2.00 mL) and H₂O (1.00 mL) were added Pd(PPh₃)₄(18.99 mg, 0.016 mmol, 0.05 equiv) and K₃PO₄(209.29 mg, 0.987 mmol, 3.00 equiv). The reaction mixture was stirred for 2 h at 80° C. under a nitrogen atmosphere, then concentrated under reduced pressure to give a residue. The residue was purified by silica gel column chromatography, eluted with CH₂Cl₂/MeOH (15:1) to afford tert-butyl 4-(6-[4,6-dimethylpyrazolo[1,5-a]pyrazin-2-yl]-1-oxo-3,4-dihydroisoquinolin-2-yl) piperidine-1-carboxylate (120 mg, 77%) as an oil. LCMS (ES, m/z): 476 [M+H]⁺.

### Synthesis of Compound 223

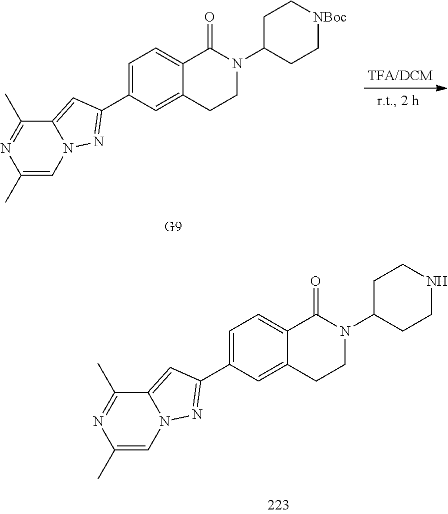 [SMILES: Cc1cn2nc(-c3ccc4c(c3)CCN(C3CCN(C(=O)OC(C)(C)C)CC3)C4=O)cc2c(C)n1 ; Cc1cn2nc(-c3ccc4c(c3)CCN(C3CCNCC3)C4=O)cc2c(C)n1]

A mixture of tert-butyl 4-(6-[4,6-dimethylpyrazolo[1,5-a]pyrazin-2-yl]-1-oxo-3,4-dihydroisoquinolin-2-yl) piperidine-1-carboxylate (100 mg, 1 equiv) and TFA (0.20 mL) in DCM (1.00 mL) was stirred for 2 h at room temperature under nitrogen atmosphere. The resulting mixture was concentrated under reduced pressure to give a residue. The residue was purified by Prep-HPLC (Condition G1, Gradient 1) to afford 6-[4,6-dimethylpyrazolo[1,5-a]pyrazin-2-yl]-2-(piperidin-4-yl)-3,4-dihydroisoquinolin-1-one (42.6 mg, 54%) as a solid. LCMS (ES, m/z): 376 [M+H]⁺. ¹H NMR (400 MHz, DMSO-d6) δ 8.49 (s, 1H), 8.02-7.92 (m, 3H), 7.53 (d, J=1.0 Hz, 1H), 4.60-4.50 (m, 1H), 3.49 (t, J=6.5 Hz, 2H), 3.02 (q, J=8.0, 6.8 Hz, 4H), 2.71 (s, 3H), 2.62-2.52 (m, 2H), 2.43 (d, J=1.0 Hz, 3H), 1.63 (qd, J=12.0, 4.0 Hz, 2H), 1.52 (d, J=10.4 Hz, 2H).

### Example G10: Synthesis of Compounds 287 and 288

### Synthesis of Intermediate G10

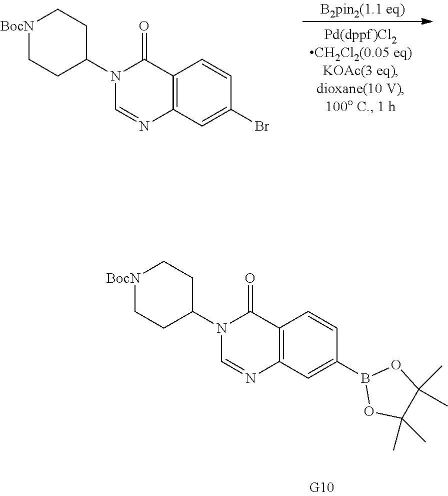 [SMILES: CC(C)(C)OC(=O)N1CCC(n2cnc3cc(Br)ccc3c2=O)CC1 ; CC(C)(C)OC(=O)N1CCC(n2cnc3cc(B4OC(C)(C)C(C)(C)O4)ccc3c2=O)CC1]

A mixture of tert-butyl 4-(7-bromo-4-oxoquinazolin-3-yl)piperidine-1-carboxylate (1.8 g, 4.41 mmol, 1.00 equiv), bis(pinacolato)diboron (1.23 g, 4.85 mmol, 1.10 equiv), KOAc (1.3 g, 13.23 mmol, 3 equiv) and Pd(dppf)Cl₂·CH₂Cl₂ (180.4 mg, 0.22 mmol, 0.05 equiv) in dioxane (18 mL) was stirred for 1 h at 100° C. under nitrogen atmosphere. The resulting mixture was filtered. LCMS (ES, m/z): 456 [M+H]⁺.

### Synthesis of Intermediate G11

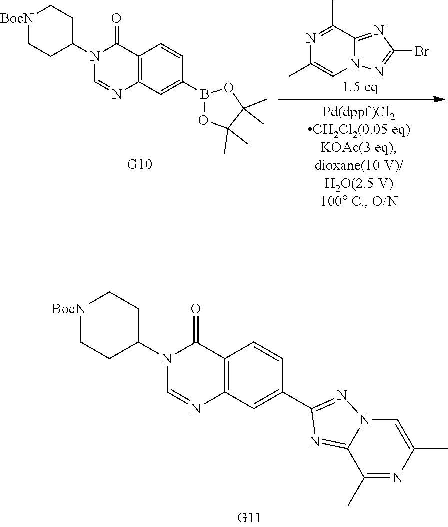

A mixture of tert-butyl 4-[4-oxo-7-(4,4,5,5-tetramethyl-1,3,2-dioxaborolan-2-yl)quinazolin-3-yl]piperidine-1-carboxylate (220.0 mg, 0.48 mmol, 1.00 equiv), 2-bromo-6,8-dimethyl-[1,2,4]triazolo[1,5-a]pyrazine (164.5 mg, 0.72 mmol, 1.5 equiv), Pd(dppf)Cl₂·CH₂Cl₂ (19.7 mg, 0.02 mmol, 0.05 equiv) and K₂CO₃ (200.3 mg, 1.45 mmol, 3 equiv) in dioxane (2 mL) and water (0.5 mL) was stirred overnight at 100° C. under nitrogen atmosphere. The resulting mixture was diluted with water (20 mL) and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by silica gel column chromatography, eluted with CH₂Cl₂/MeOH (1:0) to afford tert-butyl 4-(7-{6,8-dimethyl-[1,2,4]triazolo[1,5-a]pyrazin-2-yl}-4-oxoquinazolin-3-yl)piperidine-1-carboxylate(220.0 mg, 82%) as a solid. LCMS (ES, m/z): 476 [M+H]⁺.

### Synthesis of Compound 287

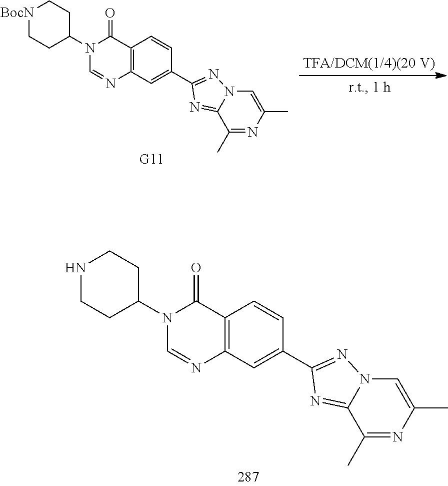 [SMILES: Cc1cn2nc(-c3ccc4c(=O)n(C5CCN(C(=O)OC(C)(C)C)CC5)cnc4c3)nc2c(C)n1 ; Cc1cn2nc(-c3ccc4c(=O)n(C5CCNCC5)cnc4c3)nc2c(C)n1]

A mixture of tert-butyl 4-(7-{6,8-dimethyl-[1,2,4]triazolo[1,5-a]pyrazin-2-yl}-4-oxoquinazolin-3-yl)piperidine-1-carboxylate (220.0 mg, 0.46 mmol, 1.00 equiv) in DCM (3 mL) and TFA (0.75 mL) was stirred for 1 h at room temperature. The resulting mixture was concentrated under vacuum to give a residue. The residue was purified by Prep-HPLC (Condition G2, Gradient 1) to afford 7-{6,8-dimethyl-[1,2,4]triazolo[1,5-a]pyrazin-2-yl}-3-(piperidin-4-yl)quinazolin-4-one (19.6 mg, 11%) as a solid.

### Synthesis of Compound 288

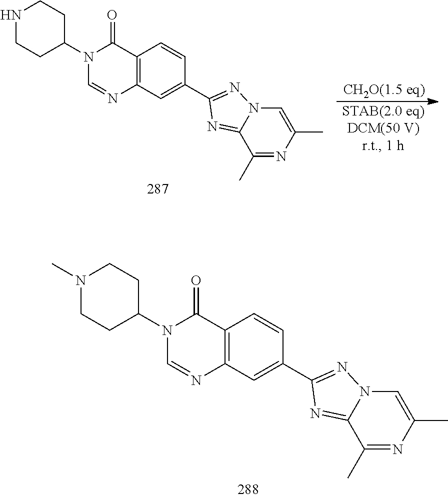

To a stirred mixture of 7-{6,8-dimethyl-[1,2,4]triazolo[1,5-a]pyrazin-2-yl}-3-(piperidin-4-yl)quinazolin-4-one (60.0 mg, 0.16 mmol, 1.00 equiv) and HCHO (48.0 mg, 1.60 mmol, 10 equiv) in DCM (3 mL) was added STAB (67.7 mg, 0.32 mmol, 2 equiv) dropwise at 0° C. The resulting mixture was stirred for 1 h at room temperature, then concentrated under vacuum to give a residue. The residue was purified by Prep-HPLC (Condition G3, Gradient 1) to afford 7-{6,8-dimethyl-[1,2,4]triazolo[1,5-a]pyrazin-2-yl}1-3-(1-methylpiperidin-4-yl)quinazolin-4-one (19.1 mg, 31%) as a solid.

| Compound Structure | Analysis data |
|---|---|
| 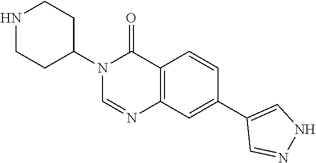 [SMILES: O=c1c2ccc(-c3cn[nH]c3)cc2ncn1C1CCNCC1] 284 | LCMS: (ES, m/z): 296 [M + H]⁺ ¹H NMR: (400 MHZ, DMSO-d₆) δ 13.13 (s, 1H), 8.41 (s, 1H), 8.31 (s, 2H), 8.11 (d, J = 8.3 Hz, 1H), 7.92 (d, J = 1.6 Hz, 1H), 7.82 (dd, J = 8.3, 1.7 Hz, 1H), 4.68 (tt, J = 12.2, 3.9 Hz, 1H), 3.14-3.06 (m, 2H), 2.63 (td, J = 12.1, 2.4 Hz, 2H), 1.92 (qd, J = 12.0, 4.1 Hz, 2H), 1.82-1.73 (m, 2H). |
| 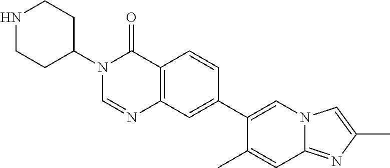 [SMILES: Cc1cn2cc(-c3ccc4c(=O)n(C5CCNCC5)cnc4c3)c(C)cc2n1] 244 | LCMS: (ES, m/z): 374 [M + H]⁺ ¹H NMR: (400 MHZ, DMSO-d₆) δ 8.48 (d, J = 13.2 Hz, 2H), 8.23 (d, J = 8.2 Hz, 1H), 7.70 (d, J = 1.7 Hz, 1H), 7.64-7.57 (m, 2H), 7.39 (s, 1H), 4.72 (ddt, J = 12.3, 8.3, 4.0 Hz, 1H), 3.10 (d, J = 11.9 Hz, 2H), 2.62 (td, J = 12.2, 2.3 Hz, 2H), 2.33 (s, 3H), 2.25 (s, 3H), 1.93 (qd, J = 11.9, 4.0 Hz, 2H), 1.79 (t, J = 6.8 Hz, 2H). |
| 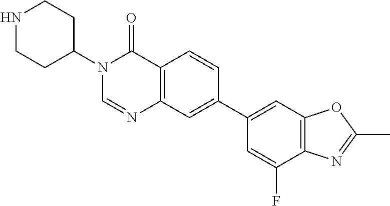 [SMILES: Cc1nc2c(F)cc(-c3ccc4c(=O)n(C5CCNCC5)cnc4c3)cc2o1] 245 | LCMS: (ES, m/z): 379 [M + H]⁺ ¹H NMR: (400 MHZ, DMSO-d₆) δ 8.50 (s, 1H), 8.24 (d, J = 8.4 Hz, 1H), 8.10 (d, J = 1.4 Hz, 1H), 8.07 (d, J = 1.8 Hz, 1H), 7.97 (dd, J = 8.4, 1.9 Hz, 1H), 7.77 (dd, J = 11.5, 1.4 Hz, 1H), 4.70 (ddt, J = 12.2, 7.8, 4.0 Hz, 1H), 3.14-3.06 (m, 2H), 2.69 (s, 3H), 2.67-2.56 (m, 2H), 1.93 (qd, J = 12.0, 4.0 Hz, 2H), 1.83-1.75 (m, 2H). ¹⁹F NMR (376 MHz, DMSO-d₆) δ −126.03. |
| 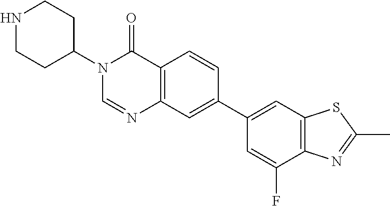 [SMILES: Cc1nc2c(F)cc(-c3ccc4c(=O)n(C5CCNCC5)cnc4c3)cc2s1] 285 | LCMS: (ES, m/z): 395 [M + H]⁺ ¹H NMR: (400 MHZ, DMSO-d₆) δ 8.46 (s, 1H), 8.38 (d, J = 1.6 Hz, 1H), 8.23 (d, J = 8.3 Hz, 1H), 8.03 (d, J = 1.8 Hz, 1H), 7.95 (dd, J = 8.4, 1.8 Hz, 1H), 7.82 (dd, J = 12.0, 1.6 Hz, 1H), 4.70 (tt, J = 12.2, 4.0 Hz, 1H), 3.12-3.03 (m, 2H), 2.85 (s, 3H), 2.59 (td, J = 12.1, 2.5 Hz, 2H), 1.90 (qd, J = 12.1, 4.1 Hz, 2H), 1.82-1.74 (m, 2H). ¹⁹F NMR (376 MHz, DMSO) δ −122.62. |
| 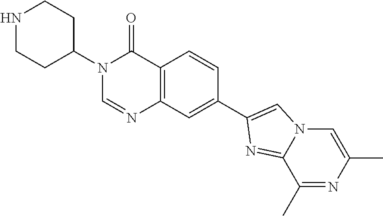 [SMILES: Cc1cn2cc(-c3ccc4c(=O)n(C5CCNCC5)cnc4c3)nc2c(C)n1] 286 | LCMS: (ES, m/z): 375 [M + H]⁺ ¹H NMR: (400 MHZ, DMSO-d₆) δ 8.71 (s, 1H), 8.46 (s, 1H), 8.28 (d, J = 1.5 Hz, 2H), 8.26-8.18 (m, 2H), 4.71 (ddt, J = 11.9, 7.6, 4.0 Hz, 1H), 3.14 (d, J = 12.5 Hz, 2H), 2.78 (s, 3H), 2.72-2.60 (m, 2H), 2.40 (s, 3H), 1.97 (qd, J = 12.1, 4.0 Hz, 2H), 1.86-1.77 (m, 2H). |
| 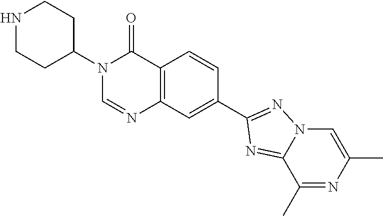 [SMILES: Cc1cn2nc(-c3ccc4c(=O)n(C5CCNCC5)cnc4c3)nc2c(C)n1] 287 | LCMS: (ES, m/z): 376 [M + H]⁺ ¹H NMR: (400 MHZ, DMSO-d₆) δ 8.74 (t, J = 0.9 Hz, 1H), 8.44 (t, J = 1.1 Hz, 1H), 8.44 (s, 1H), 8.34 (d, J = 1.0 Hz, 2H), 4.71 (tt, J = 12.1, 4.0 Hz, 1H), 3.14 (dt, J = 13.0, 3.0 Hz, 2H), 2.88 (s, 3H), 2.67 (td, J = 11.7, 2.6 Hz, 2H), 2.55 (d, J = 1.0 Hz, 3H), 1.97 (qd, J = 12.0, 4.2 Hz, 2H), 1.89- 1.80 (m, 2H). |
| 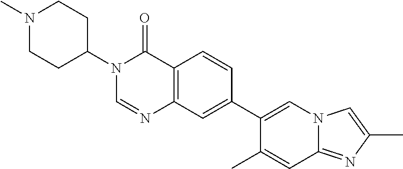 246 | LCMS: (ES, m/z): 388 [M + H]⁺ ¹H NMR: (400 MHZ, DMSO-d₆) δ 8.53 (s, 1H), 8.46 (s, 1H), 8.22 (d, J = 8.2 Hz, 1H), 7.70 (d, J = 1.7 Hz, 1H), 7.61 (d, J = 9.8 Hz, 2H), 7.39 (s, 1H), 4.62 (td, J = 11.9, 9.9, 5.8 Hz, 1H), 2.98-2.90 (m, 2H), 2.33 (s, 3H), 2.24 (d, J = 4.5 Hz, 6H), 2.17-2.01 (m, 4H), 1.85-1.77 (m, 2H). |
| 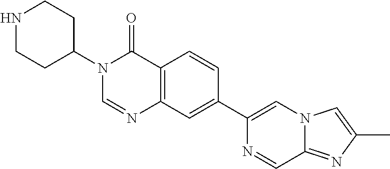 [SMILES: Cc1cn2cc(-c3ccc4c(=O)n(C5CCNCC5)cnc4c3)ncc2n1] 297 | LCMS: (ES, m/z): 361 [M + H]⁺ ¹H NMR: (400 MHZ, DMSO-d₆) δ 9.42 (d, J = 1.5 Hz, 1H), 9.07 (d, J = 1.5 Hz, 1H), 8.47 (s, 1H), 8.32 (d, J = 1.7 Hz, 1H), 8.30-8.18 (m, 2H), 7.93 (d, J = 1.0 Hz, 1H), 4.72 (tt, J = 12.1, 3.9 Hz, 1H), 3.17-3.10 (m, 2H), 2.67 (td, J = 12.5, 12.1, 2.5 Hz, 2H), 2.46 (s, 3H), 1.97 (qd, J = 12.1, 4.0 Hz, 2H), 1.86-1.77 (m, 2H). |
| 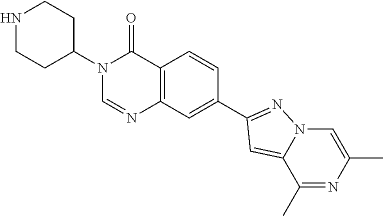 [SMILES: Cc1cn2nc(-c3ccc4c(=O)n(C5CCNCC5)cnc4c3)cc2c(C)n1] 298 | LCMS: (ES, m/z): 375 [M + H]⁺ ¹H NMR: (400 MHZ, DMSO-d₆) δ 8.54 (s, 1H), 8.45 (s, 1H), 8.30-8.23 (m, 2H), 8.19 (dd, J = 8.3, 1.6 Hz, 1H), 7.71 (d, J = 1.0 Hz, 1H), 4.73 (tt, J = 12.1, 3.9 Hz, 1H), 3.23-3.15 (m, 2H), 2.80-2.66 (m, 5H), 2.44 (d, J = 1.0 Hz, 3H), 2.02 (qd, J = 12.2, 4.0 Hz, 2H), 1.90-1.82 (m, 2H). |
| 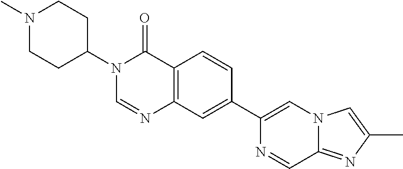 308 | LCMS: (ES, m/z): 375 [M + H]⁺ ¹H NMR: (400 MHZ, DMSO-d₆) δ 9.42 (d, J = 1.5 Hz, 1H), 9.07 (d, J = 1.4 Hz, 1H), 8.52 (s, 1H), 8.32 (d, J = 1.6 Hz, 1H), 8.29-8.18 (m, 2H), 7.93 (s, 1H), 4.61 (ddt, J = 11.7, 7.8, 3.8 Hz, 1H), 2.93 (dd, J = 9.2, 2.5 Hz, 2H), 2.46 (s, 3H), 2.23 (s, 3H), 2.12-2.00 (m, 4H), 1.86-1.77 (m, 2H). |
| 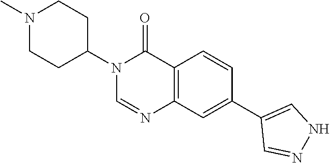 [SMILES: CN1CCC(n2cnc3cc(-c4cn[nH]c4)ccc3c2=O)CC1] 299 | LCMS: (ES, m/z): 310 [M + H]⁺ ¹H NMR: (400 MHZ, DMSO-d₆) δ 13.14 (s, 1H), 8.44 (s, 2H), 8.10 (d, J = 8.3 Hz, 2H), 7.91 (d, J = 1.7 Hz, 1H), 7.82 (dd, J = 8.3, 1.7 Hz, 1H), 4.57 (tt, J = 11.9, 4.0 Hz, 1H), 2.92 (dd, J = 9.0, 2.5 Hz, 2H), 2.22 (s, 3H), 2.18- 1.99 (m, 4H), 1.82-1.75 (m, 2H). LCMS: (ES, m/z): 393 [M + H]⁺ |
| 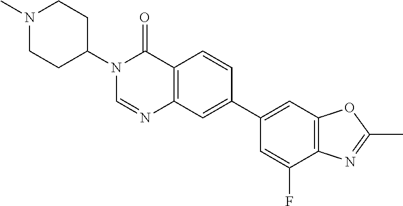 300 | H NMR: (400 MHZ, DMSO-d₆) δ 8.53 (s, 1H), 8.23 (d, J = 8.4 Hz, 1H), 8.08 (dd, J = 14.0, 1.7 Hz, 2H), 7.96 (dd, J = 8.4, 1.9 Hz, 1H), 7.76 (dd, J = 11.4, 1.5 Hz, 1H), 4.59 (td, J = 11.8, 3.9 Hz, 1H), 2.97-2.90 (m, 2H), 2.69 (s, 3H), 2.23 (s, 3H), 2.10 (ddd, J = 22.1, 11.7, 2.7 Hz, 4H), 1.81 (dd, J = 10.1, 4.0 Hz, 2H). ¹⁹F NMR (376 MHZ, DMSO) δ −126.03. |
| 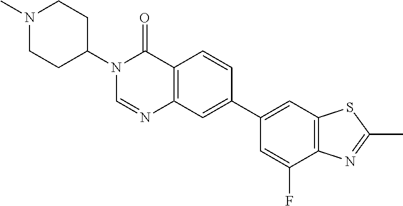 301 | LCMS: (ES, m/z): 409 [M + H]⁺ ¹H NMR: (400 MHZ, DMSO-d₆) δ 8.45 (s, 1H), 8.36 (d, J = 1.6 Hz, 1H), 8.26 (d, J = 8.3 Hz, 1H), 8.03 (d, J = 1.8 Hz, 1H), 7.94 (dd, J = 8.4, 1.9 Hz, 1H), 7.78 (dd, J = 12.1, 1.7 Hz, 1H), 4.65-4.54 (m, 1H), 2.99-2.92 (m, 2H), 2.87 (s, 3H), 2.26 (s, 3H), 2.23-2.06 (m, 4H), 1.89-1.81 (m, 2H). ¹⁹F NMR (376 MHz, DMSO) δ −122.50. |
| 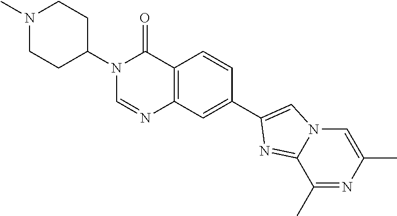 302 | LCMS: (ES, m/z): 389 [M + H]⁺ ¹H NMR: (400 MHZ, DMSO-d₆) δ 8.71 (s, 1H), 8.51 (s, 1H), 8.27 (d, J = 1.6 Hz, 2H), 8.26-8.16 (m, 2H), 4.65- 4.54 (m, 1H), 2.93 (dd, J = 11.1, 3.4 Hz, 2H), 2.78 (s, 3H), 2.40 (d, J = 1.0 Hz, 3H), 2.23 (s, 3H), 2.20-2.01 (m, 4H), 1.85-1.77 (m, 2H). |
| 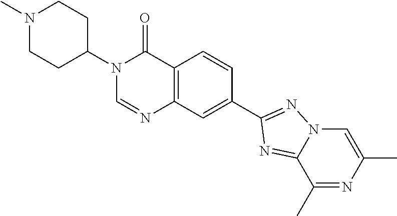 288 | LCMS: (ES, m/z): 390 [M+H] ¹H NMR: (400 MHZ, DMSO-d₆) δ 8.76 (s, 1H), 8.47 (s, 1H), 8.45 (d, J = 1.4 Hz, 1H), 8.34 (s, 2H), 4.65-4.54 (m, 1H), 2.99-2.91 (m, 2H), 2.88 (s, 3H), 2.55 (s, 3H), 2.27 (s, 3H), 2.22-2.07 (m, 4H), 1.90-1.82 (m, 2H). |
| 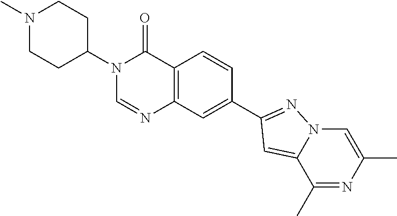 303 | LCMS: (ES, m/z): 389 [M + H]⁺ ¹H NMR: (400 MHZ, DMSO-d₆) δ 8.44 (d, J = 8.1 Hz, 2H), 8.29-8.23 (m, 2H), 8.16 (dd, J = 8.5, 1.6 Hz, 1H), 7.59 (d, J = 1.0 Hz, 1H), 4.64-4.53 (m, 1H), 2.95 (dd, J = 8.4, 2.6 Hz, 2H), 2.74 (s, 3H), 2.46 (d, J = 1.0 Hz, 3H), 2.26 (s, 3H), 2.23-2.06 (m, 4H), 1.89-1.80 (m, 2H). |

### Example G11: Synthesis of Compounds 369 and 368

### Synthesis of Intermediate G12

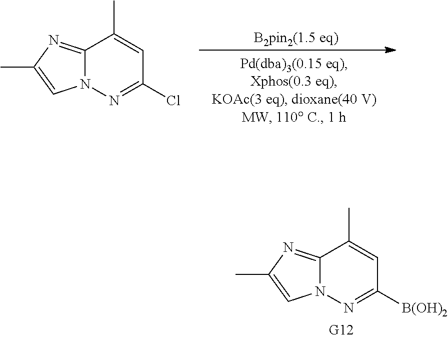

A mixture of 6-chloro-2,8-dimethylimidazo[1,2-b]pyridazine (1 g, 5.50 mmol, 1.00 equiv), B₂pin₂ (1.53 g, 6.05 mmol, 1.1 equiv), KOAc (1.62 g, 16.50 mmol, 3 equiv), X-Phos (0.26 g, 0.55 mmol, 0.1 equiv) and Pd₂(dba)₃CHCl₃ (0.28 g, 0.27 mmol, 0.05 equiv) in dioxane (30 mL) was irradiated with microwave radiation for 1 h at 110° C. The resulting mixture was filtered. LCMS (ES, m/z): 192 [M+H]⁺.

### Synthesis of Intermediate G13

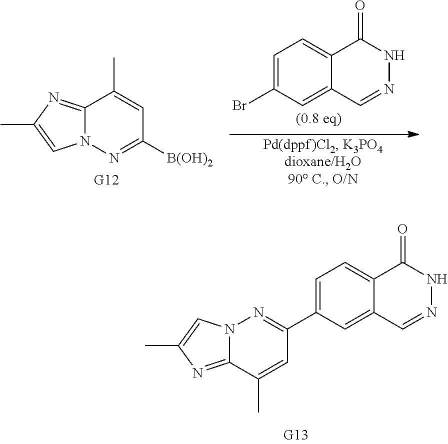 [SMILES: Cc1cn2nc(B(O)O)cc(C)c2n1 ; O=c1[nH]ncc2cc(Br)ccc12 ; Cc1cn2nc(-c3ccc4c(=O)[nH]ncc4c3)cc(C)c2n1]

A mixture of 2,8-dimethylimidazo[1,2-b]pyridazin-6-ylboronic acid (1 g, 4.77 mmol, 1.5 equiv), 6-bromo-2H-phthalazin-1-one (0.71 g, 3.18 mmol, 1.00 equiv), K₃PO₄ (2.02 g, 9.54 mmol, 3 equiv), and Pd(dppf)Cl₂CH₂Cl₂ (0.25 g, 0.31 mmol, 0.1 equiv) in dioxane (40 mL) and water (8 mL) was stirred overnight at 90° C. under nitrogen atmosphere. The reaction was quenched with water (50 mL) at room temperature, then filtered, and the filter cake washed with ethyl acetate (3×30 mL). The filtrate was concentrated under reduced pressure to afford 6-{2,8-dimethylimidazo[1,2-b]pyridazin-6-yl}-2H-phthalazin-1-one (0.80 g, 87%) as a solid. LCMS (ES, m/z): 292 [M+H]⁺.

### Synthesis of Intermediate G14

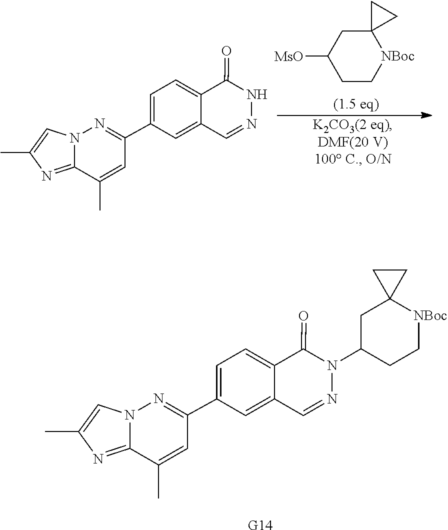 [SMILES: Cc1cn2nc(-c3ccc4c(=O)[nH]ncc4c3)cc(C)c2n1 ; CC(C)(C)OC(=O)N1CCC(OS(C)(=O)=O)CC12CC2 ; Cc1cn2nc(-c3ccc4c(=O)n(C5CCN(C(=O)OC(C)(C)C)C6(CC6)C5)ncc4c3)cc(C)c2n1 ; "G14"]

A mixture of 6-{2,8-dimethylimidazo[1,2-b]pyridazin-6-yl}-2H-phthalazin-1-one (400.0 mg, 1.37 mmol, 1.00 equiv), tert-butyl 7-(methanesulfonyloxy)-4-azaspiro[2.5]octane-4-carboxylate (628.9 mg, 2.05 mmol, 1.5 equiv), and K₂CO₃ (474.4 mg, 3.43 mmol, 2 equiv) in DMF (20 mL) was stirred overnight at 100° C. The resulting mixture was diluted with water (30 mL) and extracted with ethyl acetate (3×30 mL). The organic layers were combined, washed with brine (1×50 mL), and dried over anhydrous Na₂SO₄, and filtered. The filtrate was concentrated under reduced pressure to give a residue. The residue was purified by silica gel column chromatography, eluted with PE/EA (1:4) to afford tert-butyl 7-(6-{2,8-dimethylimidazo[1,2-b]pyridazin-6-yl}-1-oxophthalazin-2-yl)-4-azaspiro[2.5]octane-4-carboxylate (250 mg, 37%) as a solid. LCMS (ES, m/z): 501 [M+H]⁺.

### Synthesis of Compound 369 and 368

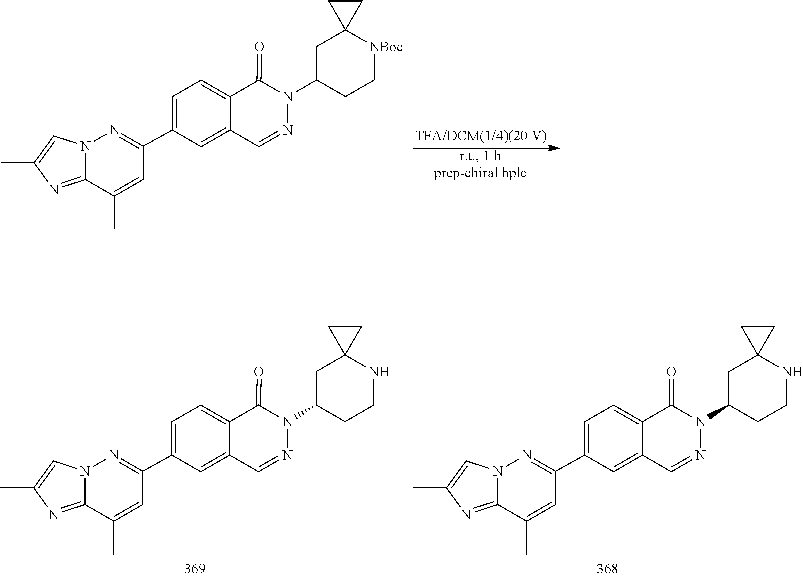 [SMILES: Cc1cn2nc(-c3ccc4c(=O)n(C5CCN(C(=O)OC(C)(C)C)C6(CC6)C5)ncc4c3)cc(C)c2n1 ; Cc1cn2nc(-c3ccc4c(=O)n([C@H]5CCNC6(CC6)C5)ncc4c3)cc(C)c2n1 ; Cc1cn2nc(-c3ccc4c(=O)n([C@@H]5CCNC6(CC6)C5)ncc4c3)cc(C)c2n1]

A mixture of tert-butyl 7-(6-{2,8-dimethylimidazo[1,2-b]pyridazin-6-yl}-1-oxophthalazin-2-yl)-4-azaspiro[2.5]octane-4-carboxylate (250.0 mg, 0.49 mmol, 1.00 equiv) in DCM (4 mL) and TFA (1 mL) was stirred for 1 h at room temperature. The resulting mixture was concentrated under vacuum to give a residue. The residue was purified by reverse flash chromatography (Condition G1, Gradient 1), followed by CHIRAL-HPLC (Condition G1, Gradient 1) to afford 2-[(7S)-4-azaspiro[2.5]octan-7-yl]-6-{2,8-dimethylimidazo[1,2-b]pyridazin-6-yl}phthalazin-1-one (53.4 mg, 27%) and 2-[(7R)-4-azaspiro[2.5]octan-7-yl]-6-{2,8-dimethylimidazo[1,2-b]pyridazin-6-yl}phthalazin-1-one (51.5 mg, 26%) as solids.

| Compound Structure | Analysis data |
|---|---|
| 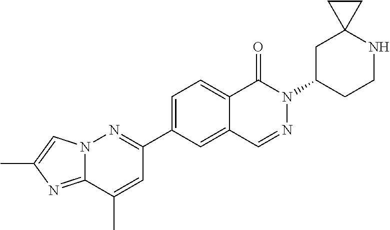 [SMILES: Cc1cn2nc(-c3ccc4c(=O)n([C@H]5CCNC6(CC6)C5)ncc4c3)cc(C)c2n1] 369 | LCMS: (ES, m/z): 401 [M + H]⁺ ¹H NMR (400 MHZ, DMSO-d₆) δ 8.64-8.56 (m, 2H), 8.49 (dd, J = 8.4, 1.8 Hz, 1H), 8.38 (d, J = 8.4 Hz, 1H), 8.13 (d, J = 1.0 Hz, 1H), 7.79 (d, J = 1.3 Hz, 1H), 5.16 (tt, J = 11.6, 4.1 Hz, 1H), 3.10-2.98 (m, 1H), 2.77 (td, J = 12.6, 2.9 Hz, 1H), 2.64 (d, J = 1.1 Hz, 3H), 2.43 (s, 3H), 2.34 (t, J = 12.0 Hz, 1H), 1.94-1.85 (m, 1H), 1.79 (d, J = 12.1 Hz, 1H), 1.24-1.15 (m, 1H), 0.66- 0.57 (m, 1H), 0.55-0.43 (m, 2H), 0.43-0.35 (m, 1H). |
| 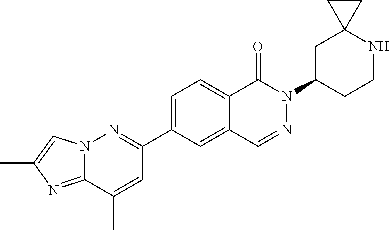 [SMILES: Cc1cn2nc(-c3ccc4c(=O)n([C@@H]5CCNC6(CC6)C5)ncc4c3)cc(C)c2n1] 368 | LCMS: (ES, m/z): 401 [M + H]⁺ ¹H NMR (400 MHZ, DMSO-d₆) δ 8.61 (d, J = 9.9 Hz, 2H), 8.50 (d, J = 8.4 Hz, 1H), 8.39 (d, J = 8.5 Hz, 1H), 8.14 (s, 1H), 7.80 (s, 1H), 5.22-5.02 (m, 1H), 3.00 (d, J = 12.8 Hz, 1H), 2.74 (d, J = 12.3 Hz, 1H), 2.65 (s, 3H), 2.43 (s, 3H), 2.31 (t, J = 11.5 Hz, 1H), 1.86 (dt, J = 11.9, 5.9 Hz, 1H), 1.77 (d, J = 11.3 Hz, 1H), 1.31-1.01 (m, 1H), 0.67-0.19 (m, 4H). |

### Example G12: Synthesis of Compound 399

### Synthesis of Compound 399

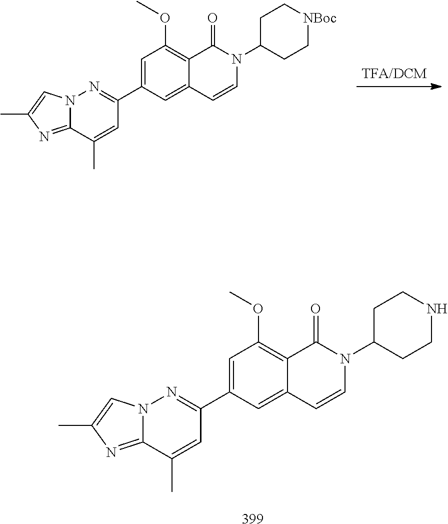 [SMILES: COc1cc(-c2cc(C)c3nc(C)cn3n2)cc2ccn(C3CCN(C(=O)OC(C)(C)C)CC3)c(=O)c12 ; COc1cc(-c2cc(C)c3nc(C)cn3n2)cc2ccn(C3CCNCC3)c(=O)c12 ; "399"]

A mixture of tert-butyl 4-(6-{2,8-dimethylimidazo[1,2-b]pyridazin-6-yl}-8-methoxy-1-oxoisoquinolin-2-yl)piperidine-1l-carboxylate (60 mg, 0.119 mmol, 1 equiv) and trifluoroacetic acid (1 mL) in DCM (5 mL) was stirred for 1 h at 0° C. The resulting mixture was concentrated under vacuum to give a residue. The residue was purified by Prep-HPLC (Condition G4, Gradient 1) to afford 6-{2,8-dimethylimidazo[1,2-b]pyridazin-6-yl}-8-methoxy-2-(piperidin-4-yl)isoquinolin-1-one (10 mg, 21%) as a solid. LCMS (ES, m/z): 404 [M+H]⁺. ¹H NMR (300 MHz, DMSO-d₆) δ 8.12 (d, J=1.0 Hz, 1H), 7.80 (dd, J=16.3, 1.4 Hz, 2H), 7.58-7.47 (m, 2H), 6.64 (d, J=7.5 Hz, 1H), 4.82 (dt, J=11.2, 6.1 Hz, 1H), 3.97 (s, 3H), 3.08 (d, J=12.0 Hz, 2H), 2.64 (d, J=1.0 Hz, 3H), 2.60 (s, 1H), 2.46-2.40 (m, 3H), 1.77-1.68 (m, 1H), 1.67 (s, 3H).

### Example G13: Synthesis of Compound 412

### Synthesis of Intermediate G15

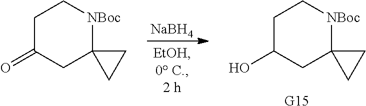

To a stirred solution of tert-butyl 7-oxo-4-azaspiro[2.5]octane-4-carboxylate (10 g, 44.388 mmol, 1 equiv) in ethanol (150 mL) was added NaBH₄ (2.5 g, 66.582 mmol, 1.5 equiv) in portions at 0° C. under nitrogen atmosphere. The resulting mixture was stirred for 2 h at room temperature under nitrogen atmosphere, then concentrated under vacuum to give a residue. The residue was partitioned between CH₂Cl₂ (20 mL) and water (50 mL). The aqueous layer was separated and extracted with CH₂Cl₂ (3×20 mL). The organic layers were combined, washed with brine (1×50 mL), dried over anhydrous Na₂SO₄, and filtered. The filtrate was concentrated under reduced pressure to afford tert-butyl 7-hydroxy-4-azaspiro[2.5]octane-4-carboxylate (10 g, 99%) as an oil. ¹H NMR (300 MHz, DMSO-d₆) δ 4.66 (d, J=4.9 Hz, 1H), 3.73 (tt, J=14.9, 4.4 Hz, 2H), 2.82 (t, J=12.5 Hz, 1H), 1.85-1.66 (m, 1H), 1.62-1.50 (m, 1H), 1.40 (s, 9H), 1.29-1.15 (m, 2H), 1.05-0.93 (m, 1H), 0.74 (ddd, J=9.5, 6.7, 5.5 Hz, 1H), 0.52-0.27 (m, 2H).

### Synthesis of Intermediate G16

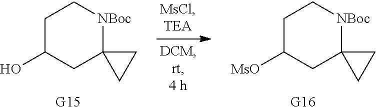

To a stirred mixture of tert-butyl 7-hydroxy-4-azaspiro[2.5]octane-4-carboxylate (11.1 g, 48.833 mmol, 1 equiv) and TEA (9.9 g, 97.666 mmol, 2 equiv) in DCM (120 mL) was added MsCl (8.4 g, 73.249 mmol, 1.5 equiv) dropwise at 0° C. under nitrogen atmosphere. The resulting mixture was stirred for 4 h at room temperature under nitrogen atmosphere, then concentrated under reduced pressure to afford tert-butyl 7-(methanesulfonyloxy)-4-azaspiro[2.5]octane-4-carboxylate (14.5 g, 97%) as an oil. ¹H NMR (300 MHz, DMSO-d₆) δ 4.89 (tt, J=9.0, 4.5 Hz, 1H), 3.71 (dt, J=13.9, 4.4 Hz, 1H), 3.20 (s, 3H), 3.10 (t, J=11.5 Hz, 1H), 2.05-1.93 (m, 1H), 1.83-1.43 (m, 3H), 1.41 (s, 9H), 0.98 (hept, J=7.2 Hz, 1H), 0.81 (ddd, J=9.7, 6.7, 5.4 Hz, 1H), 0.62 (ddd, J=9.6, 7.4, 2.9 Hz, 2H).

### Synthesis of Intermediate G17

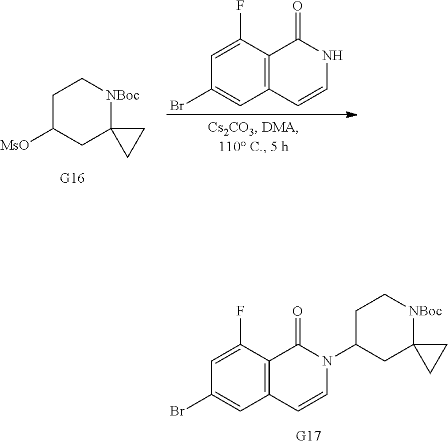

To a stirred mixture of 6-bromo-8-fluoro-2H-isoquinolin-1-one (4 g, 16.526 mmol, 1 equiv) and tert-butyl 7-(methanesulfonyloxy)-4-azaspiro[2.5]octane-4-carboxylate (6.1 g, 19.831 mmol, 1.2 equiv) in DMA (40 mL) was added Cs₂CO₃ (16.2 g, 49.578 mmol, 3 equiv) in portions at room temperature. The resulting mixture was stirred for 5 h at 100° C., then cooled to room temperature and concentrated under reduced pressure to give a residue. The residue was purified by reverse flash chromatography (column, C18 silica gel; mobile phase, ACN in water(NH₃·H₂O), 50% to 95% gradient in 12 min; detector, UV 254 nm) to afford tert-butyl 7-(6-bromo-8-fluoro-1-oxoisoquinolin-2-yl)-4-azaspiro[2.5]octane-4-carboxylate (1.4 g, 19%) as a solid. LCMS (ES, m/z): 451 [M+H]⁺.

### Synthesis of Intermediate G18

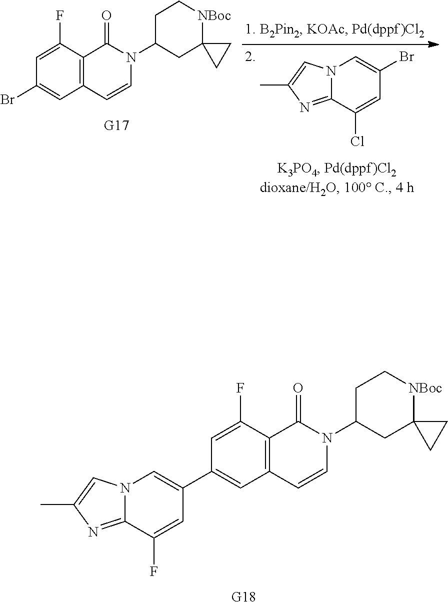

To a stirred mixture of tert-butyl 7-(6-bromo-8-fluoro-1-oxoisoquinolin-2-yl)-4-azaspiro[2.5]octane-4-carboxylate (100 mg, 0.222 mmol, 1 equiv) and bis(pinacolato)diboron (68 mg, 0.266 mmol, 1.2 equiv) in dioxane (5 mL) were added KOAc (44 mg, 0.444 mmol, 2 equiv) and Pd(dppf)Cl₂ (16 mg, 0.022 mmol, 0.1 equiv) in portions at room temperature under nitrogen atmosphere. The resulting mixture was stirred for 3 h at 100° C. under nitrogen atmosphere, then cooled to room temperature. To the reaction mixture were added 5-bromo-7-fluoro-2-methylindazole (51 mg, 0.222 mmol, 1 equiv), H₂O (1 mL), K₃PO₄ (94 mg, 0.444 mmol, 2 equiv) and Pd(dppf)Cl₂ (16 mg, 0.022 mmol, 0.1 equiv) in portions at room temperature. The resulting mixture was stirred for an additional 3 h at 100° C., then cooled to room temperature, diluted with water (10 mL), and extracted with ethyl acetate (3×5 mL). The organic layers were combined, washed with brine (1×5 mL), dried over anhydrous Na₂SO₄, and filtered. The filtrate was concentrated under reduced pressure to give a residue. The residue was purified by silica gel column chromatography, eluted with CH₂Cl₂/MeOH (10:1) to afford tert-butyl 7-[8-fluoro-6-(7-fluoro-2-methylindazol-5-yl)-1-oxoisoquinolin-2-yl]-4-azaspiro[2.5]octane-4-carboxylate (30 mg, 26%) as a solid. LCMS (ES, m/z): 521 [M+H]⁺.

### Synthesis of Compound 412

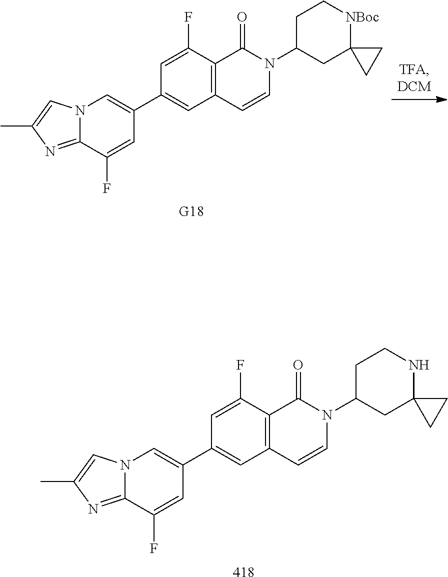 [SMILES: Cc1cn2cc(-c3cc(F)c4c(=O)n(C5CCN(C(=O)OC(C)(C)C)C6(CC6)C5)ccc4c3)cc(F)c2n1 ; "G18" ; Cc1cn2cc(-c3cc(F)c4c(=O)n(C5CCNC6(CC6)C5)ccc4c3)cc(F)c2n1 ; "418"]

A mixture of tert-butyl 7-(8-fluoro-6-{8-fluoro-2-methylimidazo[1,2-a]pyridin-6-yl}-1-oxoisoquinolin-2-yl)-4-azaspiro[2.5]octane-4-carboxylate (25 mg, 0.048 mmol, 1 equiv) in TFA (1 mL) and DCM (1 mL) was stirred for 3 h at room temperature. The resulting mixture was concentrated under reduced pressure to give a residue. The residue was basified to pH 8 with 7 M NH₃(g) in methanol. The resulting mixture was concentrated under vacuum to give a residue. The residue was purified by reverse flash chromatography (Condition G4, Gradient 2) to afford 2-{4-azaspiro[2.5]octan-7-yl}-8-fluoro-6-{8-fluoro-2-methylimidazo[1,2-a]pyridin-6-yl}isoquinolin-1-one (16 mg, 79%) as a solid. LCMS (ES, m/z): 421 [M+H]⁺. ¹H NMR (300 MHz, DMSO-d₆) δ 8.60 (d, J=2.8 Hz, 1H), 8.08 (d, J=1.4 Hz, 1H), 7.87 (d, J=1.8 Hz, 1H), 7.64 (dd, J=13.6, 1.7 Hz, 1H), 7.63-7.51 (m, 2H), 6.71 (dd, J=7.6, 2.1 Hz, 1H), 5.04 (d, J=7.9 Hz, 1H), 4.24 (s, 3H), 3.03 (d, J=12.7 Hz, 1H), 2.73 (s, 1H), 2.20 (t, J=12.0 Hz, 1H), 1.75 (s, 2H), 1.14 (dd, J=11.8, 3.9 Hz, 1H), 0.60 (t, J=6.3 Hz, 1H), 0.43 (dd, J=23.4, 6.7 Hz, 2H), 0.42 (s, 1H).

### Example G14: Synthesis of Compounds 448 and 449

### Synthesis of Intermediate G19

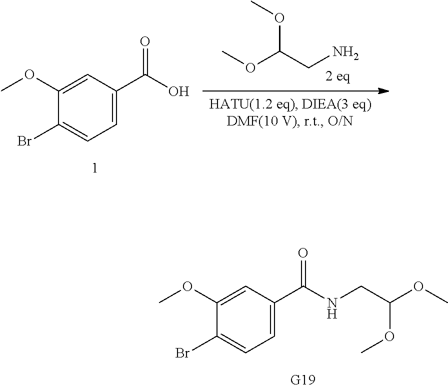

A solution of 4-bromo-3-methoxybenzoic acid (10 g, 43.282 mmol, 1 equiv), 2,2-dimethoxyethanamine (9.10 g, 86.564 mmol, 2 equiv), HATU (19.75 g, 51.938 mmol, 1.2 equiv), and DIEA (16.78 g, 129.846 mmol, 3 equiv) in DMF (100 mL) was stirred overnight at room temperature. The resulting mixture was diluted with water (100 mL) and extracted with ethyl acetate (3×100 mL). The organic layers were combined, washed with brine (3×100 mL), dried over anhydrous Na₂SO₄, and filtered. The filtrate was concentrated under reduced pressure to give a residue. The residue was purified by silica gel column chromatography, eluted with PE/EA (10:7) to afford 4-bromo-N-(2,2-dimethoxyethyl)-3-methoxybenzamide (10.56 g, 77%) as a solid.

### Synthesis of Intermediate G20

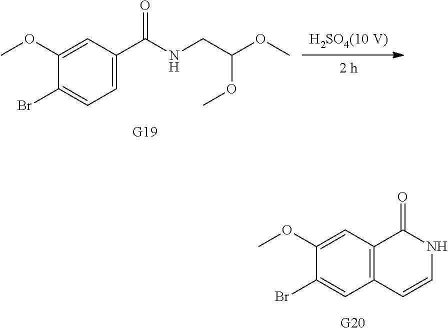

A mixture of 4-bromo-N-(2,2-dimethoxyethyl)-3-methoxybenzamide (1.5 g, 4.715 mmol, 1 equiv) in H₂SO₄ (15 mL, 281.432 mmol, 59.69 equiv) was stirred for 2 h at 60° C. The resulting mixture was diluted with water (50 mL). A precipitate formed that was collected by filtration, and the resulting solid was washed with water (3×10 mL) to afford 6-bromo-7-methoxy-2H-isoquinolin-1-one (1.12 g, 94%) as a solid.

### Synthesis of Intermediate G21

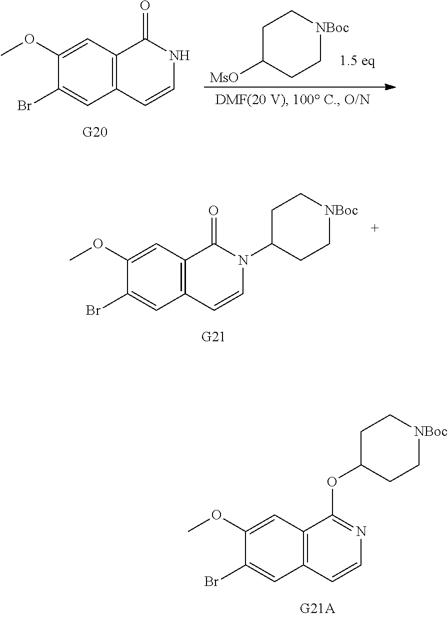 [SMILES: COc1cc2c(=O)[nH]ccc2cc1Br ; CC(C)(C)OC(=O)N1CCC(OS(C)(=O)=O)CC1 ; COc1cc2c(=O)n(C3CCN(C(=O)OC(C)(C)C)CC3)ccc2cc1Br ; COc1cc2c(OC3CCN(C(=O)OC(C)(C)C)CC3)nccc2cc1Br]

A mixture of 6-bromo-7-methoxy-1-oxo-2H-isoquinolin-2-ium (1 g, 3.920 mmol, 1 equiv), tert-butyl 4-(methanesulfonyloxy)piperidine-1-carboxylate (1.20 g, 4.312 mmol, 1.1 equiv), and K₂CO₃ (1.35 g, 9.800 mmol, 2.5 equiv) in DMF (20 mL) was stirred overnight at 100° C. The resulting mixture was diluted with water (100 mL) and extracted with ethyl acetate (3×100 mL). The organic layers were combined, washed with brine (3×100 mL), dried over anhydrous Na₂SO₄, and filtered. The filtrate was concentrated under reduced pressure to give a residue. The residue was purified by silica gel column chromatography, eluted with PE/EA (10:1) to afford tert-butyl 4-[(6-bromo-7-methoxyisoquinolin-1-yl)oxy]piperidine-1-carboxylate (570 mg, 33%) as a solid, followed by PE/EA (4:1) to afford tert-butyl 4-(6-bromo-7-methoxy-1-oxoisoquinolin-2-yl)piperidine-1-carboxylate (650 mg, 38%) as a solid.

### Synthesis of Intermediate G22

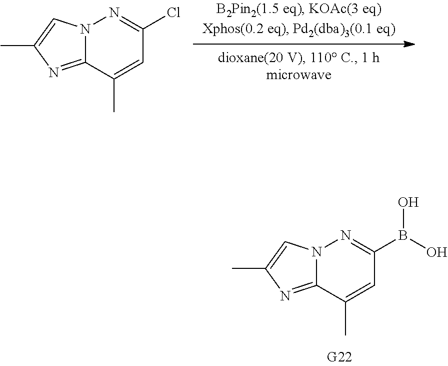

A mixture of 6-chloro-2,8-dimethylimidazo[1,2-b]pyridazine (500 mg, 2.753 mmol, 1 equiv), bis(pinacolato)diboron (1048.65 mg, 4.130 mmol, 1.5 equiv), KOAc (810.56 mg, 8.259 mmol, 3 equiv), XPhos (262.49 mg, 0.551 mmol, 0.2 equiv) and Pd₂(dba)₃·CHCl₃ (284.96 mg, 0.275 mmol, 0.1 equiv) in dioxane (10 mL) was irradiated with microwave radiation for 1 h at 110° C. under nitrogen atmosphere. The resulting mixture was filtered.

### Synthesis of Intermediate G23

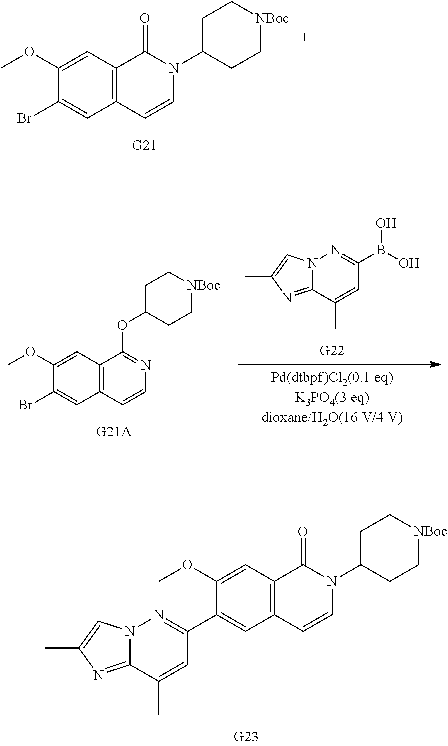 [SMILES: COc1cc2c(=O)n(C3CCN(C(=O)OC(C)(C)C)CC3)ccc2cc1Br ; COc1cc2c(OC3CCN(C(=O)OC(C)(C)C)CC3)nccc2cc1Br ; Cc1cn2nc(B(O)O)cc(C)c2n1 ; COc1cc2c(=O)n(C3CCN(C(=O)OC(C)(C)C)CC3)ccc2cc1-c1cc(C)c2nc(C)cn2n1]

A mixture of tert-butyl 4-(6-bromo-7-methoxy-1-oxoisoquinolin-2-yl)piperidine-1-carboxylate (400 mg, 0.915 mmol, 1 equiv), 2,8-dimethylimidazo[1,2-b]pyridazin-6-ylboronic acid (209.63 mg, 1.098 mmol, 1.2 equiv), K₃PO₄ (582.43 mg, 2.745 mmol, 3 equiv) and Pd(dppf)Cl₂·CH₂Cl₂ (50.52 mg, 0.092 mmol, 0.1 equiv) in dioxane (4 mL) and water (1 mL) was stirred overnight at 80° C. under nitrogen atmosphere. The resulting mixture was concentrated under vacuum to give a residue. The residue was purified by silica gel column chromatography, eluted with PE/EA (0:1) to afford tert-butyl 4-(6-{2,8-dimethylimidazo[1,2-b]pyridazin-6-yl}-7-methoxy-1-oxoisoquinolin-2-yl)piperidine-1-carboxylate (340 mg, 74%) as a solid.

### Synthesis of Compounds 448 and 449

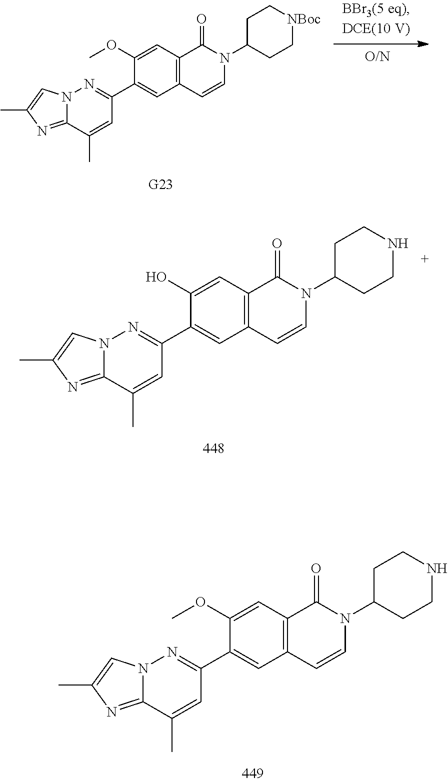 [SMILES: COc1cc2c(=O)n(C3CCN(C(=O)OC(C)(C)C)CC3)ccc2cc1-c1cc(C)c2nc(C)cn2n1 ; Cc1cn2nc(-c3cc4ccn(C5CCNCC5)c(=O)c4cc3O)cc(C)c2n1 ; COc1cc2c(=O)n(C3CCNCC3)ccc2cc1-c1cc(C)c2nc(C)cn2n1]

To a stirred solution of tert-butyl 4-(6-{2,8-dimethylimidazo[1,2-b]pyridazin-6-yl}-7-methoxy-1-oxoisoquinolin-2-yl)piperidine-1-carboxylate (150 mg, 0.298 mmol, 1 equiv) in DCE (5 mL, 63.163 mmol, 212.06 equiv) was added BBr₃ (373.09 mg, 1.490 mmol, 5 equiv) dropwise at 0° C. The resulting mixture was stirred overnight at 80° C., then neutralized to pH 9 with NH₃(g) in methanol, and concentrated under vacuum to give a residue. The residue was purified by Prep-HPLC (Condition G1, Gradient 2) to afford 6-{2,8-dimethylimidazo[1,2-b]pyridazin-6-yl}-7-hydroxy-2-(piperidin-4-yl)isoquinolin-1-one (29.1 mg, 25%) and 6-{2,8-dimethylimidazo[1,2-b]pyridazin-6-yl}-7-methoxy-2-(piperidin-4-yl)isoquinolin-1-one (17.2 mg, 43%) as solids. 448: LCMS: (ESI, m/z):390.05 [M+H]⁺. ¹HNMR: (400 MHz, DMSO-d₆) δ 8.08 (d, J=1.0 Hz, 1H), 7.88 (s, 1H), 7.82 (s, 1H), 7.48 (d, J=1.3 Hz, 1H), 7.33 (d, J=7.6 Hz, 1H), 6.68 (d, J=7.5 Hz, 1H), 4.88 (tt, J=12.0, 4.2 Hz, 1H), 3.08 (d, J=12.5 Hz, 2H), 2.64 (dd, J=12.0, 2.7 Hz, 2H), 2.58 (d, J=1.1 Hz, 3H), 2.41 (d, 3H), 1.76 (qd, J=11.8, 3.9 Hz, 2H), 1.70-1.63 (m, 2H). 449: LCMS: (ESI, m/z):404.05 [M+H]⁺. ¹H NMR: (400 MHz, DMSO-d₆) δ 8.07 (d, J=1.0 Hz, 1H), 7.83 (d, J=10.4 Hz, 2H), 7.45 (d, J=7.5 Hz, 1H), 7.31 (d, J=1.2 Hz, 1H), 6.73 (d, J=7.5 Hz, 1H), 4.95-4.86 (m, 1H), 3.92 (s, 3H), 3.09 (d, J=12.1 Hz, 2H), 2.69-2.59 (m, 2H), 2.58 (d, J=1.1 Hz, 3H), 2.41 (d, J=0.8 Hz, 3H), 1.87-1.65 (m, 4H).

### Example G15: Synthesis of Compound 453

### Synthesis of Intermediate G24

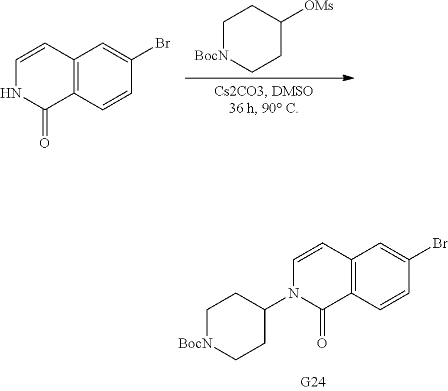 [SMILES: O=c1[nH]ccc2cc(Br)ccc12 ; CC(C)(C)OC(=O)N1CCC(OS(C)(=O)=O)CC1 ; CC(C)(C)OC(=O)N1CCC(n2ccc3cc(Br)ccc3c2=O)CC1]

To a stirred mixture of 6-bromo-2H-isoquinolin-1-one (2.2 g, 9.819 mmol, 1 equiv) and Cs₂CO₃ (6.40 g, 19.638 mmol, 2 equiv) in DMSO (20 mL) was added tert-butyl 4-(methanesulfonyloxy)piperidine-1-carboxylate (4.94 g, 17.674 mmol, 1.8 equiv) dropwise at room temperature. The resulting mixture was stirred for an additional 36 h at 90° C., then cooled to room temperature, and extracted with ethyl acetate (3×30 mL). The organic layers were combined, washed with water (3×30 mL), dried over anhydrous Na₂SO₄, and filtered. The filtrate was concentrated under reduced pressure to give a residue. The residue was purified by silica gel column chromatography, eluted with PE/EA (1:1) to afford tert-butyl 4-(6-bromo-1-oxoisoquinolin-2-yl)piperidine-1-carboxylate (1.9 g, 48%) as a solid. LCMS (ES, m/z): 407 [M+H].

### Synthesis of Intermediate G25

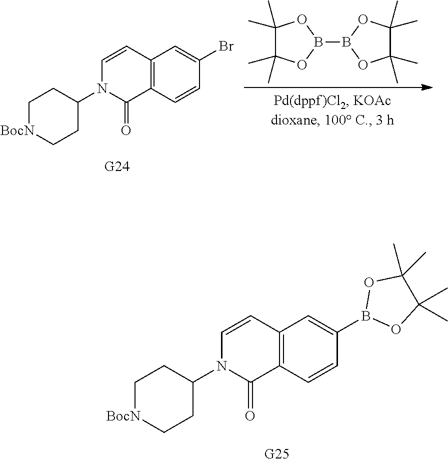

To a stirred mixture of tert-butyl 4-(6-bromo-1-oxoisoquinolin-2-yl)piperidine-1-carboxylate (450 mg, 1.105 mmol, 1 equiv) and bis(pinacolato)diboron (561.11 mg, 2.210 mmol, 2 equiv) in dioxane (20 mL) were added Pd(dppf)Cl₂·CH₂Cl₂ (90 mg, 0.111 mmol, 0.1 equiv) and KOAc (325.29 mg, 3.315 mmol, 3 equiv) dropwise at room temperature under N₂ atmosphere. The resulting mixture was stirred for an additional 3 h at 100° C., then cooled to room temperature. The resulting mixture was concentrated under reduced pressure to afford tert-butyl 4-[1-oxo-6-(4,4,5,5-tetramethyl-1,3,2-dioxaborolan-2-yl)isoquinolin-2-yl]piperidine-1-carboxylate (400 mg, 80%) as a solid. LCMS (ES, m/z): 455 [M+H].

### Synthesis of Intermediate G26

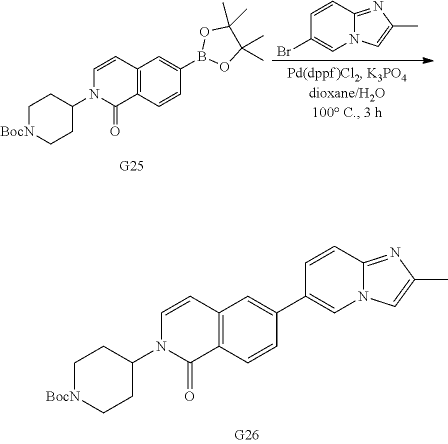

To a stirred mixture of tert-butyl 4-[1-oxo-6-(4,4,5,5-tetramethyl-1,3,2-dioxaborolan-2-yl)isoquinolin-2-yl]piperidine-1-carboxylate (400 mg, 0.880 mmol, 1 equiv) and 6-bromo-2-methylimidazo[1,2-a]pyridine (241.55 mg, 1.144 mmol, 1.3 equiv) in dioxane (16 mL) and water (4 mg) were added Pd(dppf)Cl₂·CH₂Cl₂ (71.71 mg, 0.088 mmol, 0.1 equiv) and K₃PO₄ (560.60 mg, 2.640 mmol, 3 equiv) in portions at room temperature under N₂ atmosphere. The resulting mixture was stirred for an additional 3 h at 100° C., then extracted with ethyl acetate (3×30 mL). The organic layers were combined, washed with water (3×3 mL), dried over anhydrous Na₂SO₄, and filtered. The filtrate was concentrated under reduced pressure to give a residue. The residue was purified by silica gel column chromatography, eluted with PE/EA (1:1) to afford tert-butyl 4-(6-{2-methylimidazo[1,2-a]pyridin-6-yl}-1-oxoisoquinolin-2-yl)piperidine-1-carboxylate (160 mg, 40%) as a solid. LCMS (ES, m/z): 459 [M+H].

### Synthesis of Intermediate G27

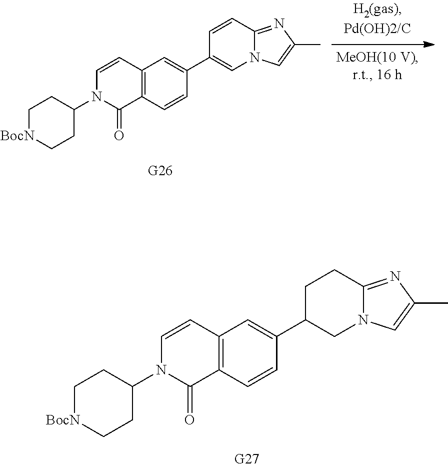 [SMILES: Cc1cn2cc(-c3ccc4c(=O)n(C5CCN(C(=O)OC(C)(C)C)CC5)ccc4c3)ccc2n1 ; Cc1cn2c(n1)CCC(c1ccc3c(=O)n(C4CCN(C(=O)OC(C)(C)C)CC4)ccc3c1)C2]

To a stirred solution of tert-butyl 4-(6-{2-methyl-5H,6H,7H,8H-imidazo[1,2-a]pyridin-6-yl}-1-oxoisoquinolin-2-yl)piperidine-1-carboxylate (128 mg, 0.277 mmol, 1 equiv) in methanol (20 mL) was added Pd(OH)₂/C (388.57 mg, 2.770 mmol, 10 equiv) in portions at room temperature under H₂ (g) atmosphere. The resulting mixture was stirred for an additional 16 h at room temperature, then filtered, and the filter cake washed with methanol (3×20 mL). The filtrate was concentrated under reduced pressure to afford tert-butyl 4-(6-{2-methylimidazo[1,2-a]pyridin-6-yl}-1-oxoisoquinolin-2-yl) piperidine-1-carboxylate (100 mg, 47%) as a solid. LCMS (ES, m/z): 463 [M+H].

### Synthesis of Compound 453

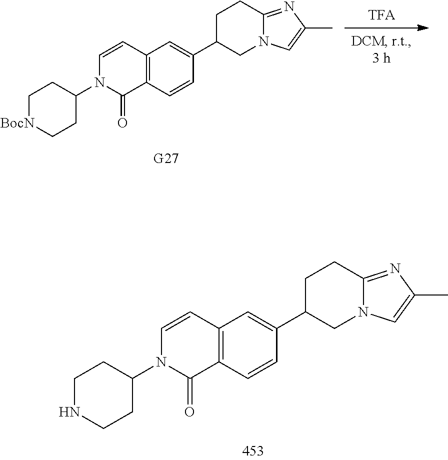 [SMILES: Cc1cn2c(n1)CCC(c1ccc3c(=O)n(C4CCN(C(=O)OC(C)(C)C)CC4)ccc3c1)C2 ; Cc1cn2c(n1)CCC(c1ccc3c(=O)n(C4CCNCC4)ccc3c1)C2]

To a stirred mixture of tert-butyl 4-(6-{2-methyl-5H,6H,7H,8H-imidazo[1,2-a]pyridin-6-yl}-1-oxoisoquinolin-2-yl)piperidine-1-carboxylate (80 mg, 0.173 mmol, 1 equiv) in DCM (9 mL) was added TFA (3 mL, 40.389 mmol, 233.55 equiv) in portions at room temperature. The reaction mixture was stirred for 3 h at room temperature, then concentrated under reduced pressure to give a residue. The residue was purified by Prep-HPLC (Condition G2, Gradient 2) to afford 6-{2-methyl-5H,6H,7H,8H-imidazo[1,2-a]pyridin-6-yl}-2-(piperidin-4-yl)isoquinolin-1-one (25.7 mg, 41%) as a solid. LCMS (ES, m/z): 363 [M+H]. ¹H NMR (400 MHz, DMSO-d₆) δ 8.19 (d, J=8.3 Hz, 1H), 7.59 (s, 1H), 7.49 (t, J=7.8 Hz, 2H), 6.68 (s, 1H), 6.60 (d, J=7.5 Hz, 1H), 4.86 (t, J=13.1 Hz, 1H), 4.21-4.10 (m, 1H), 3.92 (d, J=11.8 Hz, 1H), 3.38 (s, 1H), 3.07 (d, J=10.2 Hz, 2H), 2.84-2.78 (m, 2H), 2.13 (q, J=7.8, 7.2 Hz, 2H), 2.05 (s, 3H), 1.75 (d, J=10.9 Hz, 2H), 1.68 (s, 2H).

### Example G16: Synthesis of Compound 454

### Synthesis of Intermediate G28

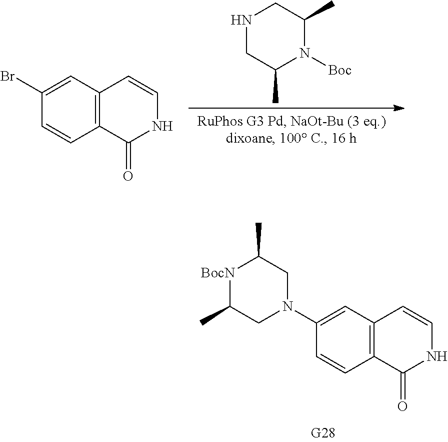 [SMILES: O=c1[nH]ccc2cc(Br)ccc12 ; C[C@@H]1CNC[C@H](C)N1C(=O)OC(C)(C)C ; C[C@@H]1CN(c2ccc3c(=O)[nH]ccc3c2)C[C@H](C)N1C(=O)OC(C)(C)C]

To a mixture of compound 6-bromo-2H-isoquinolin-1-one (400 mg, 1.785 mmol, 1 equiv) and tert-butyl (2R,6S)-2,6-dimethylpiperazine-1-carboxylate (382.60 mg, 1.785 mmol, 1 equiv) in 1,4-dioxane (10 mL) were added RuPhos Palladacycle Gen.3 (149.32 mg, 0.178 mmol, 0.1 equiv) and t-BuONa (514.72 mg, 5.355 mmol, 3 equiv). The reaction mixture was stirred for 16 h at 100° C., then cooled to room temperature, diluted with water (60 mL), and extracted with ethyl acetate (3×60 mL). The organic layers were combined, washed with brine (1×50 mL), dried over anhydrous Na₂SO₄, and filtered. The filtrate was concentrated under reduced pressure to give a residue. The residue was purified by silica gel column chromatography, eluted with PE/EA (30%-60%) to afford tert-butyl (2R,6S)-2,6-dimethyl-4-(1-oxo-2H-isoquinolin-6-yl)piperazine-1-carboxylate (360 mg, 56%). LCMS (ESI, m/z): 358[M+H]⁺.

### Synthesis of Intermediate G29

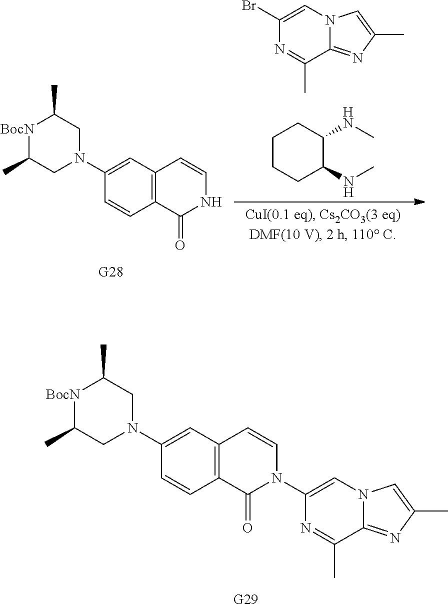

To a mixture of compound tert-butyl (2R,6S)-2,6-dimethyl-4-(1-oxo-2H-isoquinolin-6-yl)piperazine-1-carboxylate (300 mg, 0.839 mmol, 1 equiv) and 6-bromo-2,8-dimethylimidazo[1,2-a]pyrazine (208.71 mg, 0.923 mmol, 1.1 equiv) in DMF (10 mL) were added (1S,2S)—N1,N₂-dimethylcyclohexane-1,2-diamine (9.55 mg, 0.067 mmol, 0.08 equiv), CuI (5.33 mg, 0.084 mmol, 0.1 equiv) and cesium carbonate (820.35 mg, 2.517 mmol, 3 equiv). The reaction mixture was stirred for 2 h at 110° C., then cooled to room temperature. The resulting mixture was diluted with water (60 mL) and extracted with ethyl acetate (3×60 mL). The organic layers were combined, washed with brine (1×50 mL), dried over anhydrous Na₂SO₄, and filtered. The filtrate was concentrated under reduced pressure to give a residue. The residue was purified by silica gel column chromatography, eluted with MeOH/DCM (1:10) to afford tert-butyl (2R,6S)-4-(2-{2,8-dimethylimidazo[1,2-a]pyrazin-6-yl}-1-oxoisoquinolin-6-yl)-2,6-dimethylpiperazine-1-carboxylate (215 mg, 51%). LCMS (ESI, m/z): 503[M+H]⁺.

### Synthesis of Compound 454

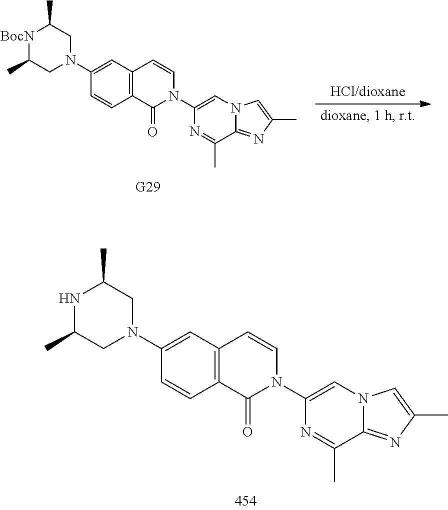

To a solution of tert-butyl (2R,6S)-4-(2-{2,8-dimethylimidazo[1,2-a]pyrazin-6-yl}-1-oxoisoquinolin-6-yl)-2,6-dimethylpiperazine-1-carboxylate (60 mg, 0.119 mmol, 1 equiv) in dioxane (3 mL) was added HCl (gas) in 1,4-dioxane (1.0 M, 1 mL). The reaction mixture was stirred for 1 h at 25° C. under a nitrogen atmosphere, then concentrated under reduced pressure to give a residue. The residue was purified by Prep-HPLC (Condition G2, Gradient 1) to afford 2-{2,8-dimethylimidazo[1,2-a]pyrazin-6-yl}-6-[(3R,5S)-3,5-dimethylpiperazin-1-yl]isoquinolin-1-one (28.1 mg, 58%) as a solid. LCMS (ESI, m/z): 403 [M+H]⁺. ¹H NMR (400 MHz, DMSO-d₆) δ 8.87 (s, 1H), 8.07-7.95 (m, 2H), 7.57 (d, J=7.5 Hz, 1H), 7.21 (dd, J=9.2, 2.4 Hz, 1H), 7.01 (d, J=2.4 Hz, 1H), 6.58 (d, J=7.6 Hz, 1H), 3.83 (d, J=13.9 Hz, 2H), 2.83 (dt, J=9.4, 4.5 Hz, 2H), 2.74 (s, 3H), 2.44 (s, 3H), 2.34-2.27 (m, 2H), 1.05 (d, J=6.3 Hz, 6H).

### Example G17: Synthesis of Compound 509

### Synthesis of Intermediate G30

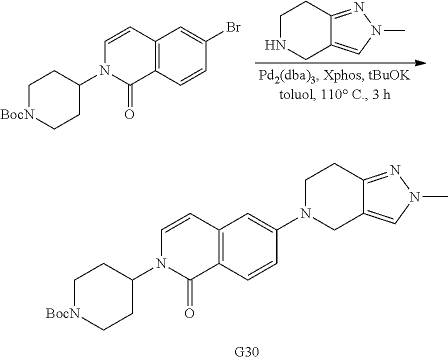

A mixture of 2-methyl-4H,5H,6H,7H-pyrazolo[4,3-c]pyridine (67.36 mg, 0.491 mmol, 1 equiv) and tert-butyl 4-(6-bromo-1-oxoisoquinolin-2-yl)piperidine-1-carboxylate (200 mg, 0.491 mmol, 1.00 equiv) in toluene (4 mL) was treated with Xphos (46.82 mg, 0.098 mmol, 0.2 equiv), t-BuOK (165.30 mg, 1.473 mmol, 3 equiv), and Pd₂(dba)₃ (44.96 mg, 0.049 mmol, 0.1 equiv) in toluene (1 mL) under nitrogen atmosphere. The reaction mixture was stirred for 3 h at 110° C., then quenched with water at room temperature, and extracted with ethyl acetate (3×4 mL). The organic layers were combined, dried over anhydrous Na₂SO₄, and filtered. The filtrate was concentrated under reduced pressure to give a residue. The residue was purified by silica gel column chromatography, eluted with PE/EA (10:1) to afford tert-butyl 4-(6-{2-methyl-4H,6H,7H-pyrazolo[4,3-c]pyridin-5-yl}-1-oxoisoquinolin-2-yl)piperidine-1-carboxylate (200 mg, 88%) as a solid.

### Synthesis of Compound 509

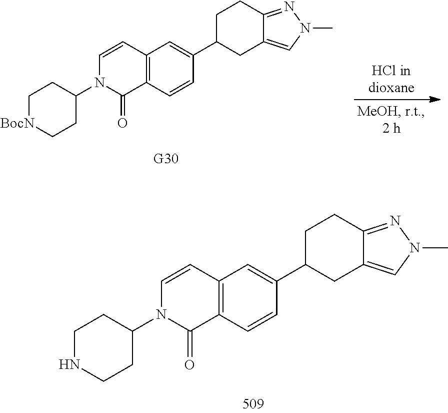 [SMILES: Cn1cc2c(n1)CCC(c1ccc3c(=O)n(C4CCN(C(=O)OC(C)(C)C)CC4)ccc3c1)C2 ; Cn1cc2c(n1)CCC(c1ccc3c(=O)n(C4CCNCC4)ccc3c1)C2]

A mixture of tert-butyl 4-(6-{2-methyl-4H,6H,7H-pyrazolo[4,3-c]pyridin-5-yl}-1-oxoisoquinolin-2-yl)piperidine-1-carboxylate (200 mg, 0.431 mmol, 1 equiv) and HCl (gas) in 1,4-dioxane (1 mL) in methanol (1 mL) was stirred for 2 h at room temperature under nitrogen atmosphere. The resulting mixture was concentrated under reduced pressure to give a residue. The residue was purified by Prep-HPLC (Condition G1, Gradient 2) to afford 6-{2-methyl-4H,6H,7H-pyrazolo[4,3-c]pyridin-5-yl}-2-(piperidin-4-yl)isoquinolin-1-one (42.8 mg, 27%) as a solid. LCMS (ES, m/z): 364[M+H]⁺. ¹H NMR (400 MHz, DMSO-d₆) δ 8.01 (d, J=9.0 Hz, 1H), 7.49 (s, 1H), 7.33 (d, J=7.6 Hz, 1H), 7.20 (dd, J=9.1, 2.5 Hz, 1H), 6.96 (d, J=2.6 Hz, 1H), 6.46 (d, J=7.5 Hz, 1H), 4.87-4.74 (m, 1H), 4.40 (s, 2H), 3.75 (d, J=3.6 Hz, 5H), 3.05 (d, J=12.2 Hz, 2H), 2.72 (t, J=5.8 Hz, 2H), 2.64-2.54 (m, 2H), 1.76-1.58 (m, 4H).

### Example G18: Synthesis of Compound 521

### Synthesis of Intermediate G31

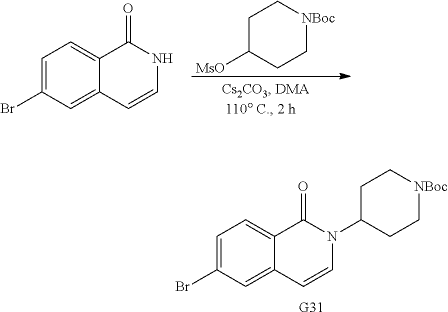

To a stirred mixture of 6-bromo-2H-isoquinolin-1-one (1.0 g, 4.463 mmol, 1.0 equiv) and tert-butyl 4-(methanesulfonyloxy)piperidine-1-carboxylate (2.49 g, 8.926 mmol, 2.0 equiv) in DMA (10 mL) was added Cs₂CO₃ (4.36 g, 13.389 mmol, 3.0 equiv) at room temperature. The resulting mixture was stirred for 2 h at 110° C., then cooled to room temperature and extracted with ethyl acetate (3×20 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with EA to afford tert-butyl 4-(6-bromo-1-oxoisoquinolin-2-yl)piperidine-1-carboxylate (670 mg, 37%) as a solid. LCMS (ES, m/z): 407 [M+H]⁺.

### Synthesis of Intermediate G32

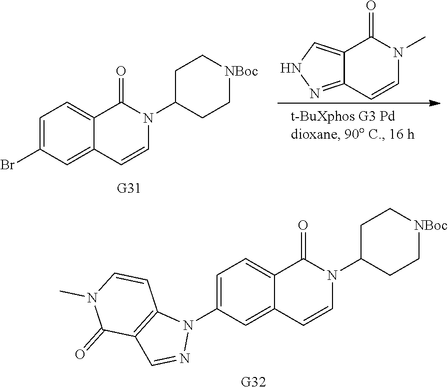 [SMILES: CC(C)(C)OC(=O)N1CCC(n2ccc3cc(Br)ccc3c2=O)CC1 ; Cn1ccc2n[nH]cc2c1=O ; Cn1ccc2c(cnn2-c2ccc3c(=O)n(C4CCN(C(=O)OC(C)(C)C)CC4)ccc3c2)c1=O ; "G32"]

To a stirred mixture of tert-butyl 4-(6-bromo-1-oxoisoquinolin-2-yl)piperidine-1-carboxylate (200 mg, 0.491 mmol, 1.0 equiv) and 5-methyl-2H-pyrazolo[4,3-c]pyridin-4-one (95.2 mg, 0.638 mmol, 1.3 equiv) in dioxane (2 mL) were added Cs₂CO₃ (479.9 mg, 1.473 mmol, 3.0 equiv) and t-BuXphos Pd G3 (39.0 mg, 0.049 mmol, 0.1 equiv) at room temperature. The resulting mixture was stirred overnight at 90° C. under nitrogen atmosphere, then concentrated under reduced pressure to give a residue. The residue was purified by silica gel column chromatography, eluted with EA to afford tert-butyl 4-(6-{5-methyl-4-oxopyrazolo[4,3-c]pyridin-1-yl}-1-oxoisoquinolin-2-yl)piperidine-1-carboxylate (160 mg, 69%) as a solid. LCMS (ES, m/z): 476 [M+H]⁺.

### Synthesis of Intermediate G33

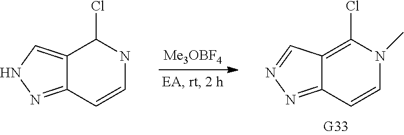

A solution of 4-chloro-2H-pyrazolo[4,3-c]pyridine (1 g, 6.512 mmol, 1 equiv) in ethyl acetate (10 mL) was treated with Me₃OBF₄ (4.82 g, 32.560 mmol, 5 equiv) at room temperature. The resulting mixture was stirred for 2 h at room temperature, then neutralized to pH 7 with saturated NaHCO₃ (aq.), and extracted with ethyl acetate (3×20 mL). The organic layers were combined, washed with brine (1×10 mL), dried over anhydrous Na₂SO₄ land filtered. The filtrate was concentrated under reduced pressure to afford 4-chloro-5-methylpyrazolo[4,3-c]pyridine (900 mg, 82%) as a solid. LCMS (ES, m/z): 168 [M+H]⁺.

### Synthesis of Intermediate G34

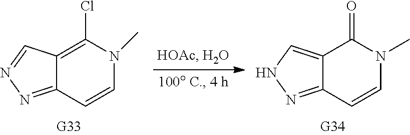

A solution of 4-chloro-5-methylpyrazolo[4,3-c]pyridine (900 mg, 5.370 mmol, 1 equiv) in water (3 mL) was treated with HOAc (3 mL). The resulting mixture was stirred for 4 h at 100° C., then cooled to room temperature and concentrated under reduced pressure to afford 5-methyl-2H-pyrazolo[4,3-c]pyridin-4-one (778 mg, 97%) as a solid. LCMS (ES, m/z): 150 [M+H]⁺.

### Synthesis of Compound 521

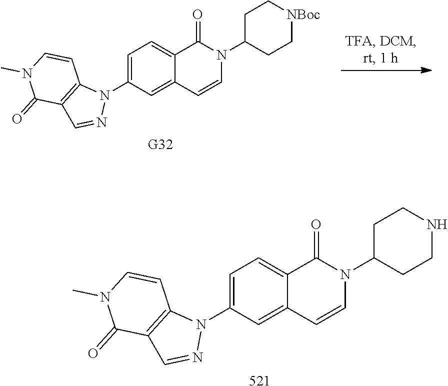 [SMILES: Cn1ccc2c(cnn2-c2ccc3c(=O)n(C4CCN(C(=O)OC(C)(C)C)CC4)ccc3c2)c1=O ; "G32" ; Cn1ccc2c(cnn2-c2ccc3c(=O)n(C4CCNCC4)ccc3c2)c1=O ; "521"]

A solution of tert-butyl 4-{7-[(7-fluoro-2-methyl-3H-1,3-benzodiazol-5-yl)carbamoyl]-2-methylindazol-4-yl}piperazine-1-carboxylate (50 mg, 0.099 mmol, 1.0 equiv) in DCM (0.8 mL) was treated with TFA (0.2 mL) for 1 h at room temperature. The resulting mixture was concentrated under reduced pressure to give a residue. The residue was purified by Prep-HPLC (Condition G4, Gradient 3) to afford N-(7-fluoro-2-methyl-3H-1,3-benzodiazol-5-yl)-2-methyl-4-(piperazin-1-yl)indazole-7-carboxamide (14.2 mg, 35%) as a solid. LCMS (ES, m/z): 376 [M+H]⁺. ¹H NMR (400 MHz, DMSO-d₆) δ 8.41 (t, J=4.4 Hz, 2H), 8.00 (d, J=2.2 Hz, 1H), 7.84 (dd, J=8.7, 2.2 Hz, 1H), 7.71 (d, J=7.5 Hz, 1H), 7.62 (d, J=7.5 Hz, 1H), 6.89 (d, J=7.5 Hz, 1H), 6.83 (d, J=7.5 Hz, 1H), 4.89-4.86 (m, 1H), 3.53 (s, 3H), 3.09 (d, J=12.2 Hz, 2H), 2.63 (t, J=11.8 Hz, 2H), 1.91-1.64 (m, 4H).

### Example G19: Synthesis of Compound 522

### Synthesis of Compound 522

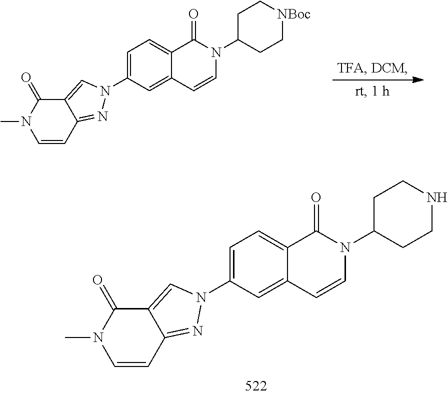 [SMILES: Cn1ccc2nn(-c3ccc4c(=O)n(C5CCN(C(=O)OC(C)(C)C)CC5)ccc4c3)cc2c1=O ; Cn1ccc2nn(-c3ccc4c(=O)n(C5CCNCC5)ccc4c3)cc2c1=O]

A solution of tert-butyl 4-(6-{5-methyl-4-oxopyrazolo[4,3-c]pyridin-2-yl}-1-oxoisoquinolin-2-yl)piperidine-1-carboxylate (18 mg, 0.038 mmol, 1.0 equiv) in DCM (0.2 mL) was treated with TFA (0.1 mL). The reaction mixture was stirred for 1 h at room temperature, then concentrated under reduced pressure to give a residue. The residue was purified by Prep-HPLC (Condition G4, Gradient 3) to afford 6-{5-methyl-4-oxopyrazolo[4,3-c]pyridin-2-yl}-2-(piperidin-4-yl)isoquinolin-1-one (1.4 mg, 10%) as a solid. LCMS (ES, m/z): 376 [M+H]⁺. ¹H NMR (400 MHz, DMSO-d₆) δ 9.48 (s, 1H), 8.35 (dd, J=5.6, 3.3 Hz, 2H), 8.18 (dd, J=8.9, 2.2 Hz, 1H), 7.60 (d, J=7.6 Hz, 1H), 7.43 (d, J=7.5 Hz, 1H), 6.74 (d, J=7.5 Hz, 1H), 6.58 (d, J=7.5 Hz, 1H), 4.85 (d, J=12.4 Hz, 1H), 3.45 (s, 3H), 3.08 (d, J=12.1 Hz, 2H), 2.62 (t, J=11.6 Hz, 2H), 1.91-1.64 (m, 4H).

### Example G20: Synthesis of Compound 530

### Synthesis of Intermediate G35

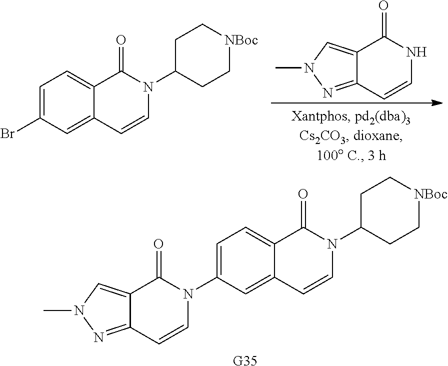

To a stirred mixture of tert-butyl 4-(6-bromo-1-oxoisoquinolin-2-yl)piperidine-1-carboxylate (100 mg, 0.246 mmol, 1.0 equiv) and 2-methyl-5H-pyrazolo[4,3-c]pyridin-4-one (43.9 mg, 0.295 mmol, 1.2 equiv) in dioxane (1 mL) were added Cs₂CO₃ (239.9 mg, 0.738 mmol, 3.0 equiv), Xantphos (28.4 mg, 0.049 mmol, 0.2 equiv), and Pd₂(dba)₃ (22.5 mg, 0.025 mmol, 0.1 equiv) at room temperature. The resulting mixture was stirred overnight at 100° C. under nitrogen atmosphere, then concentrated under reduced pressure to give a residue. The residue was purified by silica gel column chromatography, eluted with EA to afford tert-butyl 4-(6-{2-methyl-4-oxopyrazolo[4,3-c]pyridin-5-yl}-1-oxoisoquinolin-2-yl)piperidine-1-carboxylate (89 mg, 76%) as a solid. LCMS (ES, m/z): 476 [M+H]⁺.

### Synthesis of Intermediate G36

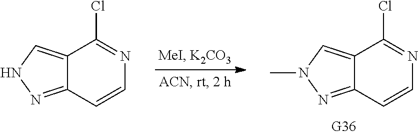

To a stirred mixture of 4-chloro-2H-pyrazolo[4,3-c]pyridine (1 g, 6.512 mmol, 1.0 equiv) and K₂CO₃ (2.70 g, 19.536 mmol, 3.0 equiv) in acetonitrile (10 mL) was added methyl iodide (1.39 g, 9.768 mmol, 1.5 equiv) dropwise at 0° C. The resulting mixture was stirred for 2 h at room temperature, then extracted with ethyl acetate (3×50 mL). The organic layers were combined, washed with brine (1×30 mL), dried over anhydrous Na₂SO₄, and filtered. The filtrate was concentrated under reduced pressure to afford 4-chloro-2-methylpyrazolo[4,3-c]pyridine (370 mg, 34%) as a solid. LCMS (ES, m/z): 168 [M+H]⁺.

### Synthesis of Intermediate G37

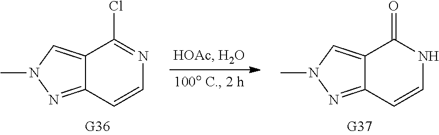

A solution of 4-chloro-2-methylpyrazolo[4,3-c]pyridine (350 mg, 2.088 mmol, 1 equiv) in water (1.5 mL) was treated with HOAc (1.5 mL). The resulting mixture was stirred for 4 h at 100° C., then cooled to room temperature and concentrated under reduced pressure to afford 2-methyl-5H-pyrazolo[4,3-c]pyridin-4-one (310 mg, 100 as a solid. LCMS (ES, m/z): 150 [M+H]⁺.

### Synthesis of Compound 530

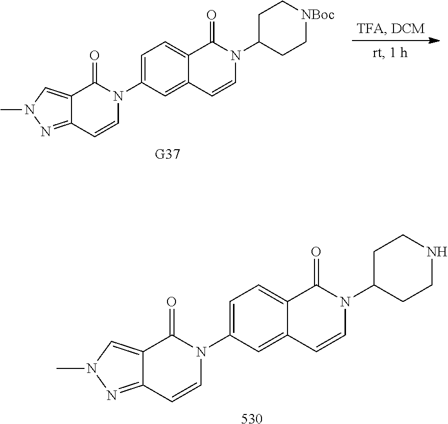 [SMILES: Cn1cc2c(=O)n(-c3ccc4c(=O)n(C5CCN(C(=O)OC(C)(C)C)CC5)ccc4c3)ccc2n1 ; "G37" ; Cn1cc2c(=O)n(-c3ccc4c(=O)n(C5CCNCC5)ccc4c3)ccc2n1 ; "530"]

A solution of tert-butyl 4-(6-{2-methyl-4-oxopyrazolo[4,3-c]pyridin-5-yl}-1-oxoisoquinolin-2-yl)piperidine-1-carboxylate (70 mg, 0.147 mmol, 1 equiv) in DCM (0.4 mL) was treated with TFA (0.2 mL). The reaction mixture was stirred for 1 h at room temperature, then concentrated under reduced pressure to give a residue. The residue was purified by Prep-HPLC (Condition G5, Gradient 1) to afford 6-{2-methyl-4-oxopyrazolo[4,3-c]pyridin-5-yl}-2-(piperidin-4-yl)isoquinolin-1-one (28.4 mg, 51%) as a solid. LCMS (ES, m/z): 376 [M+H]⁺. ¹H NMR (400 MHz, DMSO-d₆) δ 8.60 (s, 1H), 8.31 (d, J=8.6 Hz, 1H), 7.74 (d, J=2.1 Hz, 1H), 7.58 (d, J=7.6 Hz, 1H), 7.53 (dd, J=8.6, 2.1 Hz, 1H), 7.39 (d, J=7.6 Hz, 1H), 6.72 (d, J=7.5 Hz, 1H), 6.63 (d, J=7.6 Hz, 1H), 4.97-4.82 (m, 1H), 4.06 (s, 3H), 3.09 (d, J=12.3 Hz, 2H), 2.63 (t, J=11.7 Hz, 2H), 1.85-1.63 (m, 4H).

### Example G21: Synthesis of Compound 558

### Synthesis of Intermediate G38

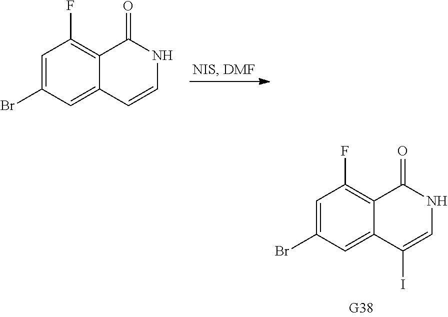 [SMILES: O=c1[nH]ccc2cc(Br)cc(F)c12 ; O=c1[nH]cc(I)c2cc(Br)cc(F)c12]

To a stirred solution of 6-bromo-8-fluoroisoquinolin-1(2H)-one (800 mg, 3.306 mmol, 1.0 equiv) in DMF (5 mL) was added NIS (893 mg, 3.967 mmol, 1.2 equiv) in portions at room temperature. The resulting mixture was stirred for 2 h at room temperature, then quenched with water (20 mL). A precipitate formed that was collected by filtration and washed with water (2×10 mL). The resulting solid was dried to afford 6-bromo-8-fluoro-4-iodoisoquinoline-1(2H)-one (1.0 g, 82%) as a solid. LCMS (ES, m/z): 368 [M+H]⁺.

### Synthesis of Intermediate G39

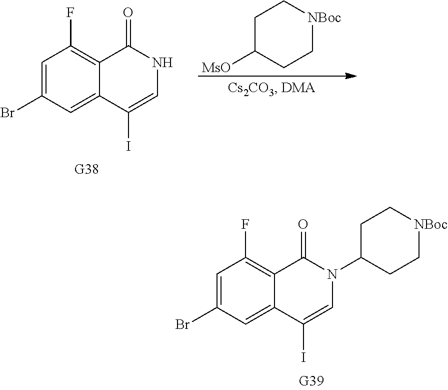 [SMILES: O=c1[nH]cc(I)c2cc(Br)cc(F)c12 ; CC(C)(C)OC(=O)N1CCC(OS(C)(=O)=O)CC1 ; CC(C)(C)OC(=O)N1CCC(n2cc(I)c3cc(Br)cc(F)c3c2=O)CC1]

To a stirred mixture of 6-bromo-8-fluoro-4-iodoisoquinoline-1(2H)-one (1.0 g, 2.725 mmol, equiv) and tert-butyl 4-((methylsulfonyl)oxy)piperidine-1-carboxylate (913 mg, 3.270 mmol, 1.2 equiv) in DMA (20 mL) was added Cs₂CO₃ (1.78 g, 5.450 mmol, 2.0 equiv) in portions at room temperature. The resulting mixture was stirred for 2 h at 100° C., then quenched with water (50 mL) and extracted with ethyl acetate (3×20 mL). The organic layers were combined, washed with brine (2×20 mL), dried over anhydrous Na₂SO₄, and filtered. The filtrate was concentrated under reduced pressure to give a residue. The residue was purified by silica gel column chromatography, eluted with PE:EA (2:1) to afford tert-butyl 4-(6-bromo-8-fluoro-4-iodo-1-oxoisoquinolin-2(1H)-yl)piperidine-1-carboxylate (0.6 g, 40%) as a solid. LCMS (ES, m/z): 552 [M+H]⁺.

### Synthesis of Intermediate G40

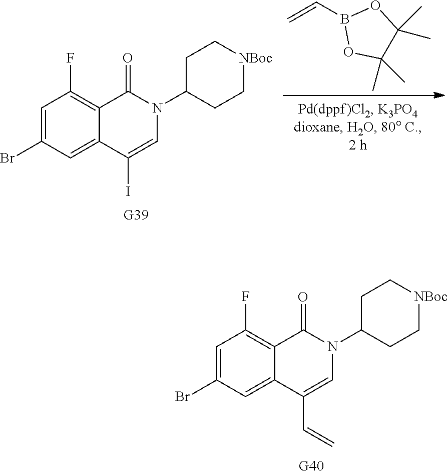 [SMILES: CC(C)(C)OC(=O)N1CCC(n2cc(I)c3cc(Br)cc(F)c3c2=O)CC1 ; C=CB1OC(C)(C)C(C)(C)O1 ; C=Cc1cn(C2CCN(C(=O)OC(C)(C)C)CC2)c(=O)c2c(F)cc(Br)cc12]

To a mixture of tert-butyl 4-(6-bromo-8-fluoro-4-iodo-1-oxoisoquinolin-2-yl)piperidine-1-carboxylate (400 mg, 0.726 mmol, 1 equiv) and 2-ethenyl-4,4,5,5-tetramethyl-1,3,2-dioxaborolane (134.13 mg, 0.871 mmol, 1.2 equiv) in dioxane (5.0 mL) and water (0.5 mL) were added K₃PO₄ (308.08 mg, 1.452 mmol, 2.0 equiv) and Pd(dppf)Cl₂ (53 mg, 0.073 mmol, 0.1 equiv). The reaction mixture was stirred for 2 h at 80° C. under a nitrogen atmosphere, then concentrated under reduced pressure to give a residue. The residue was purified by silica gel column chromatography, eluted with PE:EA (10:1) to afford tert-butyl 4-(6-bromo-4-ethenyl-8-fluoro-1-oxoisoquinolin-2-yl)piperidine-1-carboxylate (200 mg, 61%) as a solid. LCMS (ES, m/z): 453 [M+H]⁺.

### Synthesis of Intermediate G41

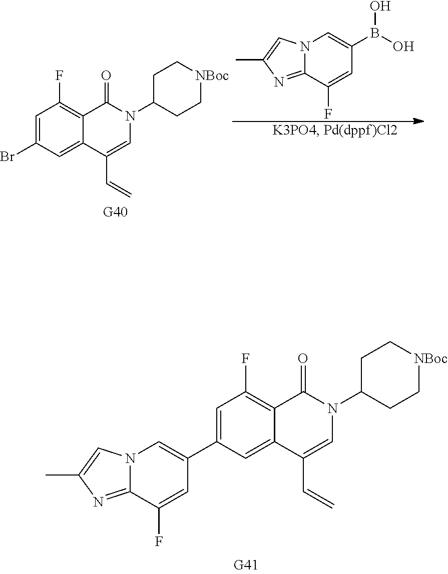 [SMILES: C=Cc1cn(C2CCN(C(=O)OC(C)(C)C)CC2)c(=O)c2c(F)cc(Br)cc12 ; Cc1cn2cc(B(O)O)cc(F)c2n1 ; C=Cc1cn(C2CCN(C(=O)OC(C)(C)C)CC2)c(=O)c2c(F)cc(-c3cc(F)c4nc(C)cn4c3)cc12]

To a stirred mixture of tert-butyl 4-(6-bromo-4-ethenyl-8-fluoro-1-oxoisoquinolin-2-yl)piperidine-1-carboxylate (100 mg, 0.222 mmol, 1 equiv) and 8-fluoro-2-methylimidazo[1,2-a]pyridin-6-ylboronic acid (64 mg, 0.333 mmol, 1.5 equiv) in dioxane (3 mL) were added K₃PO₄ (141 mg, 0.666 mmol, 3 equiv) and Pd(dppf)Cl₂ (16.21 mg, 0.022 mmol, 0.1 equiv) at room temperature under nitrogen atmosphere. The resulting mixture was stirred for 2 h at 100° C. under nitrogen atmosphere, then concentrated under reduced pressure to give a residue. The residue was purified by silica gel column chromatography, eluted with PE/EA (1:1) to afford tert-butyl 4-(4-ethenyl-8-fluoro-6-{8-fluoro-2-methylimidazo[1,2-a]pyridin-6-yl}-1-oxoisoquinolin-2-yl)piperidine-1-carboxylate (80 mg, 70%) as a solid. LCMS (ES, m/z): 521 [M+H]⁺.

### Synthesis of Intermediate G42

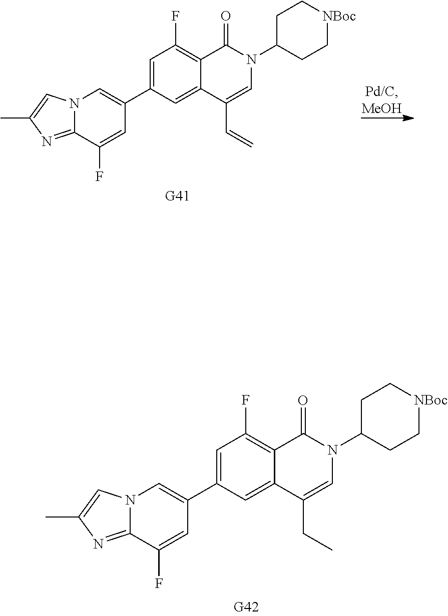 [SMILES: C=Cc1cn(C2CCN(C(=O)OC(C)(C)C)CC2)c(=O)c2c(F)cc(-c3cc(F)c4nc(C)cn4c3)cc12 ; CCc1cn(C2CCN(C(=O)OC(C)(C)C)CC2)c(=O)c2c(F)cc(-c3cc(F)c4nc(C)cn4c3)cc12]

To a solution of tert-butyl 4-(4-ethenyl-8-fluoro-6-{8-fluoro-2-methylimidazo[1,2-a]pyridin-6-yl}-1-oxoisoquinolin-2-yl)piperidine-1-carboxylate (50 mg, 0.096 mmol, 1 equiv) in methanol (5 mL) was added Pd/C (10%, 21 mg) under nitrogen atmosphere. The reaction mixture was hydrogenated at room temperature for 3 h under hydrogen atmosphere using a hydrogen balloon, then filtered through a celite, and the filtrate concentrated under reduced pressure to afford tert-butyl 4-(4-ethyl-8-fluoro-6-{8-fluoro-2-methylimidazo[1,2-a]pyridin-6-yl}-1-oxoisoquinolin-2-yl)piperidine-1-carboxylate (40 mg, 80%) as a solid. LCMS (ES, m/z): 523 [M+H]⁺.

### Synthesis of Compound 558

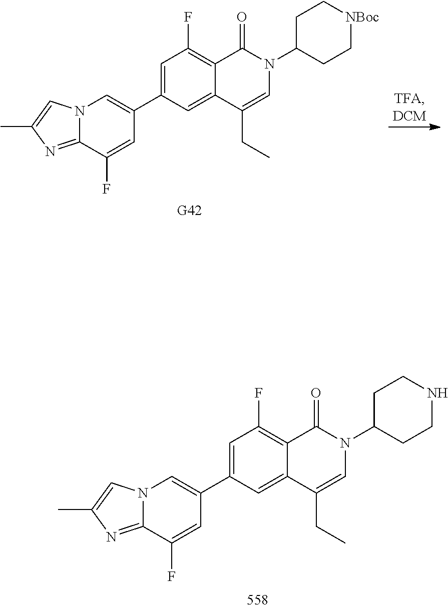 [SMILES: CCc1cn(C2CCN(C(=O)OC(C)(C)C)CC2)c(=O)c2c(F)cc(-c3cc(F)c4nc(C)cn4c3)cc12 ; CCc1cn(C2CCNCC2)c(=O)c2c(F)cc(-c3cc(F)c4nc(C)cn4c3)cc12]

A mixture of tert-butyl 4-(4-ethyl-8-fluoro-6-{8-fluoro-2-methylimidazo[1,2-a]pyridin-6-yl}-1-oxoisoquinolin-2-yl)piperidine-1-carboxylate (50 mg, 0.096 mmol, 1 equiv) in TFA (1 mL) and DCM (3 mL) was stirred for 2 h at room temperature. The resulting mixture was concentrated under reduced pressure to give a residue. The residue was basified to pH 8 with 7 M NH₃(g) in methanol, then concentrated under vacuum to give a residue. The residue was purified by reverse flash chromatography (Condition G1, Gradient 1) to afford 4-ethyl-8-fluoro-6-{8-fluoro-2-methylimidazo[1,2-a]pyridin-6-yl}-2-(piperidin-4-yl)isoquinolin-1-one (10 mg, 25%) as a solid. LCMS (ES, m/z): 423 [M+H]⁺. ¹H NMR (400 MHz, Methanol-d₄) δ 8.76 (d, J=1.4 Hz, 1H), 7.81 (d, J=1.8 Hz, 2H), 7.62-7.51 (m, 2H), 7.34 (s, 1H), 5.07 (dq, J=11.2, 6.2, 5.8 Hz, 1H), 3.25 (d, J=12.9 Hz, 2H), 2.96-2.78 (m, 4H), 2.50-2.45 (m, 3H), 2.00-1.86 (m, 4H), 1.37 (t, J=7.4 Hz, 3H).

### Example G22: Synthesis of Compound 559

### Synthesis of Compound 559

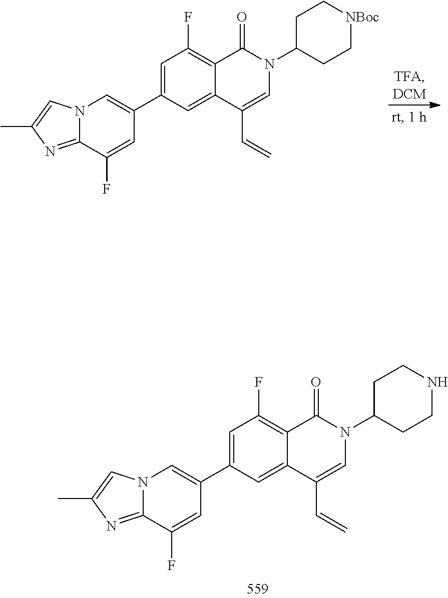 [SMILES: C=Cc1cn(C2CCN(C(=O)OC(C)(C)C)CC2)c(=O)c2c(F)cc(-c3cc(F)c4nc(C)cn4c3)cc12 ; C=Cc1cn(C2CCNCC2)c(=O)c2c(F)cc(-c3cc(F)c4nc(C)cn4c3)cc12]

A mixture of tert-butyl 4-(4-ethenyl-8-fluoro-6-{8-fluoro-2-methylimidazo[1,2-a]pyridin-6-yl}-1-oxoisoquinolin-2-yl)piperidine-1-carboxylate (20 mg, 0.038 mmol, 1 equiv) and TFA (0.1 mL) in DCM (1.0 mL) was stirred for 1 h at room temperature. The resulting mixture was basified to pH 8 with NH₃ (g) in methanol, then concentrated under reduced pressure to give a residue. The residue was purified by reverse flash chromatography (Condition G2, Gradient 1) to afford 4-ethenyl-8-fluoro-6-{8-fluoro-2-methylimidazo[1,2-a]pyridin-6-yl}-2-(piperidin-4-yl)isoquinolin-1-one (3 mg, 19%) as a solid. LCMS (ES, m/z): 421 [M+H]⁺. ¹H NMR (300 MHz, Methanol-d₄) δ 8.77 (d, J=1.5 Hz, 1H), 7.89-7.77 (m, 2H), 7.66-7.51 (m, 3H), 7.24-7.09 (m, 1H), 5.72 (dd, J=17.1, 1.5 Hz, 1H), 5.46 (dd, J=10.8, 1.5 Hz, 1H), 5.12-5.01 (m, 1H), 3.26 (dd, J=10.8, 8.0 Hz, 2H), 2.94-2.79 (m, 2H), 2.48 (d, J=1.0 Hz, 3H), 1.93 (td, J=9.3, 7.8, 3.5 Hz, 4H).

### Example G23: Synthesis of Compound 560

### Synthesis of Intermediate G43

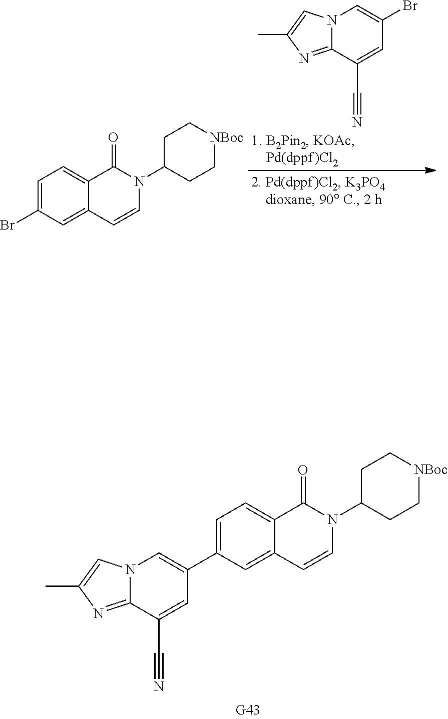 [SMILES: Cc1cn2cc(Br)cc(C#N)c2n1 ; CC(C)(C)OC(=O)N1CCC(n2ccc3cc(Br)ccc3c2=O)CC1 ; Cc1cn2cc(-c3ccc4c(=O)n(C5CCN(C(=O)OC(C)(C)C)CC5)ccc4c3)cc(C#N)c2n1]

To a stirred mixture of 6-bromo-2-methylimidazo[1,2-a]pyridine-8-carbonitrile (150 mg, 0.635 mmol, 1.00 equiv) and bis(pinacolato)diboron (177.49 mg, 0.699 mmol, 1.1 equiv) in dioxane (5 mL) were added KOAc (124.72 mg, 1.270 mmol, 2 equiv) and Pd(dppf)Cl₂ (46.49 mg, 0.064 mmol, 0.1 equiv) in portions at room temperature under nitrogen atmosphere. The resulting mixture was stirred for 2 h at 90° C. under nitrogen atmosphere, then cooled to room temperature. To the reaction mixture was added tert-butyl 4-(6-bromo-1-oxoisoquinolin-2-yl)piperidine-1-carboxylate (155.28 mg, 0.381 mmol, 0.6 equiv), water (1 mL), K₃PO₄ (269.75 mg, 1.270 mmol, 2 equiv), and Pd(dppf)Cl₂ (46.49 mg, 0.064 mmol, 0.1 equiv) in portions at room temperature under nitrogen atmosphere. The resulting mixture was stirred for an additional 2 h at 90° C., then quenched with water (30 mL) at room temperature, and extracted with ethyl acetate (3×10 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with PE/EA (1:1) to afford tert-butyl 4-(6-{8-cyano-2-methylimidazo[1,2-a]pyridin-6-yl}-1-oxoisoquinolin-2-yl)piperidine-1-carboxylate (230 mg, 75%) as a solid. LCMS (ES, m/z): 484 [M+H]⁺.

### Synthesis of Compound 560

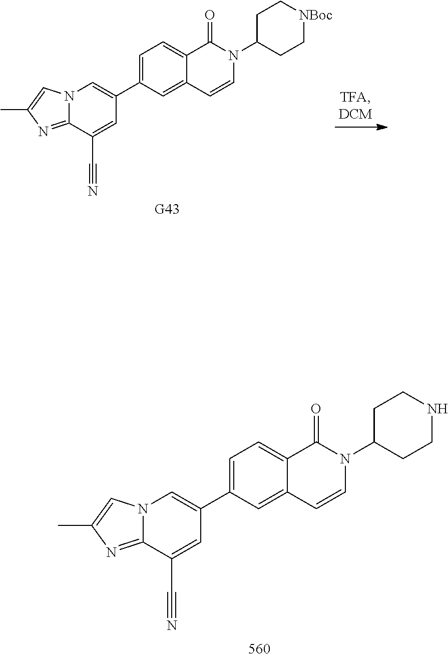 [SMILES: Cc1cn2cc(-c3ccc4c(=O)n(C5CCN(C(=O)OC(C)(C)C)CC5)ccc4c3)cc(C#N)c2n1 ; Cc1cn2cc(-c3ccc4c(=O)n(C5CCNCC5)ccc4c3)cc(C#N)c2n1]

A mixture of tert-butyl 4-(6-{8-cyano-2-methylimidazo[1,2-a]pyridin-6-yl}-1-oxoisoquinolin-2-yl)piperidine-1-carboxylate (100 mg, 0.207 mmol, 1 equiv) and TFA (1 mL) in DCM (3 mL) was stirred for 2 h at room temperature. The resulting mixture was concentrated under reduced pressure to give a residue. The residue was purified by reverse flash chromatography (Condition G1, Gradient 4) to afford 2-methyl-6-[1-oxo-2-(piperidin-4-yl)isoquinolin-6-yl]imidazo[1,2-a]pyridine-8-carbonitrile) as a solid. LCMS (ES, m/z): 384 [M+H]⁺. ¹H NMR (300 MHz, DMSO-d₆) δ 9.36 (d, J=1.8 Hz, 1H), 8.43 (d, J=1.7 Hz, 1H), 8.32 (d, J=8.5 Hz, 1H), 8.09 (d, J=1.8 Hz, 1H), 7.96-7.84 (m, 2H), 7.58 (d, J=7.5 Hz, 1H), 6.70 (d, J=7.5 Hz, 1H), 5.15-4.53 (m, 1H), 3.09 (d, J=11.8 Hz, 2H), 2.65 (d, J=11.0 Hz, 2H), 2.43 (s, 3H), 1.77 (dd, J=11.4, 3.8 Hz, 1H), 1.71 (s, 3H).

### Example G24: Synthesis of Compound 587

### Synthesis of Intermediate G44

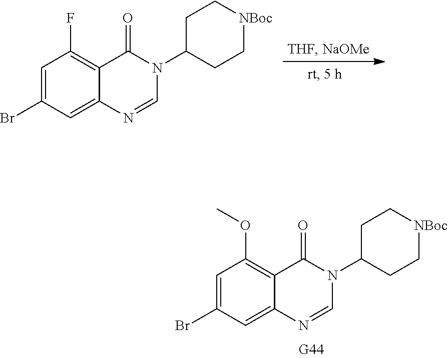 [SMILES: CC(C)(C)OC(=O)N1CCC(n2cnc3cc(Br)cc(F)c3c2=O)CC1 ; COc1cc(Br)cc2ncn(C3CCN(C(=O)OC(C)(C)C)CC3)c(=O)c12]

To a mixture of tert-butyl 4-(7-bromo-5-fluoro-4-oxoquinazolin-3-yl)piperidine-1-carboxylate (220 mg, 0.516 mmol, 1 equiv) and THF (3 mL) was added NaOMe (33.46 mg, 0.619 mmol, 1.2 equiv) dropwise at room temperature. The resulting mixture was stirred for 5 h at room temperature, then quenched with a mixture of water and ice (5 mL) at room temperature, and extracted with CH₂Cl₂ (3×10 mL). The organic layers were combined, dried over anhydrous Na₂SO₄, and filtered. The filtrate was concentrated under reduced pressure to afford tert-butyl 4-(7-bromo-5-methoxy-4-oxoquinazolin-3-yl)piperidine-1-carboxylate (220 mg, 97%) as a solid. LCMS (ES, m/z): 438 [M+H]⁺.

### Synthesis of Intermediate G45

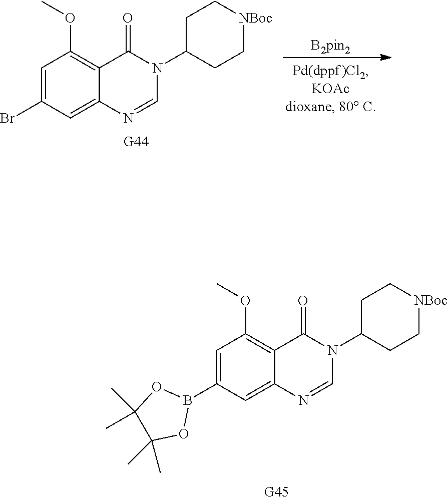 [SMILES: COc1cc(Br)cc2ncn(C3CCN(C(=O)OC(C)(C)C)CC3)c(=O)c12 ; COc1cc(B2OC(C)(C)C(C)(C)O2)cc2ncn(C3CCN(C(=O)OC(C)(C)C)CC3)c(=O)c12]

A mixture of tert-butyl 4-(7-bromo-5-methoxy-4-oxoquinazolin-3-yl)piperidine-1-carboxylate (220 mg, 0.502 mmol, 1 equiv), 4,4,5,5-tetramethyl-2-(tetramethyl-1,3,2-dioxaborolan-2-yl)-1,3,2-dioxaborolane (152.95 mg, 0.602 mmol, 1.2 equiv), AcOK (147.78 mg, 1.506 mmol, 3.0 equiv), dioxane (2 mL) and Pd(dppf)Cl₂ (40.89 mg, 0.050 mmol, 0.1 equiv) was stirred for 4 h at 80° C. under nitrogen atmosphere. LCMS (ES, m/z): 486 [M+H]⁺.

### Synthesis of Intermediate G46

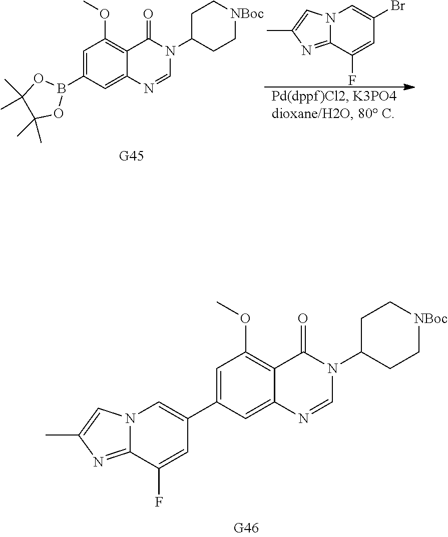

To the stirred mixture of Intermediate G45 were added 6-bromo-8-fluoro-2-methylimidazo[1,2-a]pyridine (103.82 mg, 0.453 mmol, 1.0 equiv), K₃PO₄ (96.21 mg, 0.453 mmol, 1.0 equiv), water (0.4 mL), and Pd(dppf)Cl₂ (36.92 mg, 0.045 mmol, 0.1 equiv) at room temperature under nitrogen atmosphere. The resulting mixture was stirred for 4 h at 80° C. under nitrogen atmosphere, then concentrated under reduced pressure to give a residue. The residue was purified by silica gel column chromatography, eluted with CH₂Cl₂/MeOH (10:1) to afford tert-butyl 4-(7-{8-fluoro-2-methylimidazo[1,2-a]pyridin-6-yl}-5-methoxy-4-oxoquinazolin-3-yl)piperidine-1-carboxylate (150 mg, 65%) as a solid.

### Synthesis of Compound 587

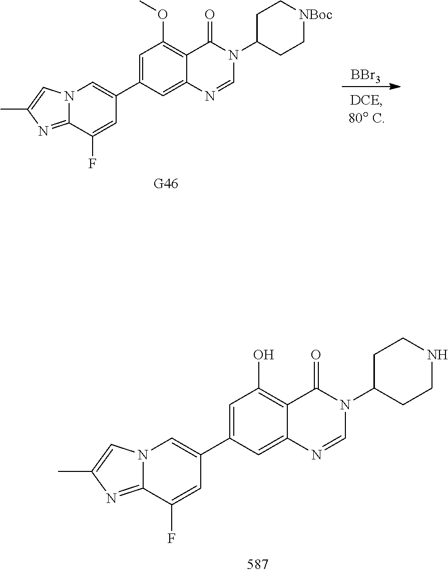

A mixture of tert-butyl 4-(7-{8-fluoro-2-methylimidazo[1,2-a]pyridin-6-yl}-5-methoxy-4-oxoquinazolin-3-yl)piperidine-1-carboxylate (90 mg, 0.177 mmol, 1 equiv), DCE (1 mL), and boron tribromide (133.26 mg, 0.531 mmol, 3 equiv) was stirred overnight at 80° C., then quenched with methanol (5 mL) at room temperature and concentrated under reduced pressure to give a residue. The residue was purified by Prep-HPLC (Condition G6, Gradient 1) to afford 7-{8-fluoro-2-methylimidazo[1,2-a]pyridin-6-yl}-5-hydroxy-3-(piperidin-4-yl)quinazolin-4-one (15 mg, 22%) as a solid. LCMS (ES, m/z): 394 [M+H]⁺. ¹H NMR (400 MHz, DMSO-d₆) δ 11.81 (s, 1H), 9.08 (d, J=3.1 Hz, 1H), 8.78 (s, 1H), 8.42 (s, 1H), 8.34 (s, 1H), 7.89 (s, 1H), 7.82 (t, J=10.0 Hz, 1H), 7.57 (d, J=1.7 Hz, 1H), 7.35 (d, J=1.7 Hz, 1H), 4.82 (t, J=12.2 Hz, 1H), 3.49 (d, J=12.5 Hz, 2H), 3.17 (d, J=12.1 Hz, 1H), 3.11 (d, J=12.2 Hz, 1H), 2.42 (s, 3H), 2.34 (d, J=12.6 Hz, 2H), 2.13 (d, J=13.2 Hz, 2H).

### Example G25: Synthesis of Compound 588

### Synthesis of Intermediate G47

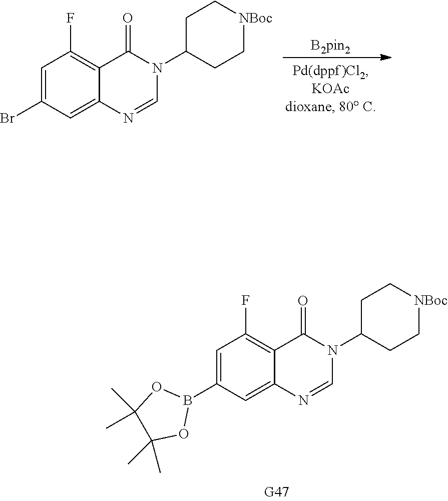 [SMILES: CC(C)(C)OC(=O)N1CCC(n2cnc3cc(Br)cc(F)c3c2=O)CC1 ; CC(C)(C)OC(=O)N1CCC(n2cnc3cc(B4OC(C)(C)C(C)(C)O4)cc(F)c3c2=O)CC1]

A mixture of tert-butyl 4-(7-bromo-5-fluoro-4-oxoquinazolin-3-yl)piperidine-1-carboxylate (100 mg, 0.235 mmol, 1.0 equiv), 4,4,5,5-tetramethyl-2-(tetramethyl-1,3,2-dioxaborolan-2-yl)-1,3,2-dioxaborolane (71.48 mg, 0.282 mmol, 1.2 equiv), AcOK (69.07 mg, 0.705 mmol, 3 equiv), dioxane (1 mL), and Pd(dppf)Cl₂ (19.11 mg, 0.024 mmol, 0.1 equiv) was stirred for 4 h at 80° C. under nitrogen atmosphere. LCMS (ES, m/z): 474 [M+H]⁺.

### Synthesis of Intermediate G48

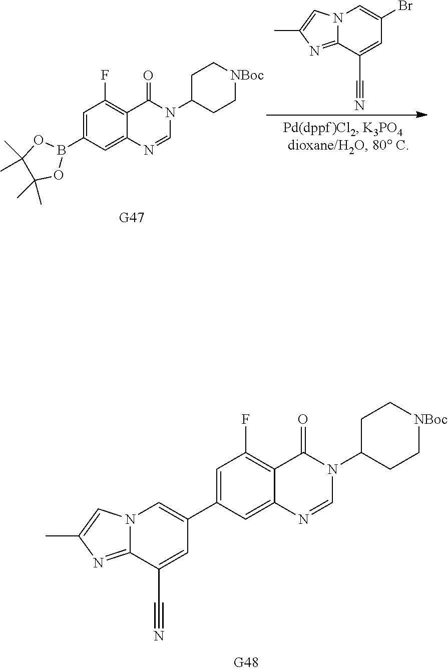 [SMILES: CC(C)(C)OC(=O)N1CCC(n2cnc3cc(B4OC(C)(C)C(C)(C)O4)cc(F)c3c2=O)CC1 ; Cc1cn2cc(Br)cc(C#N)c2n1 ; Cc1cn2cc(-c3cc(F)c4c(=O)n(C5CCN(C(=O)OC(C)(C)C)CC5)cnc4c3)cc(C#N)c2n1]

To a stirred mixture of Intermediate G47 were added water (0.2 mL), 6-bromo-2-methylimidazo[1,2-a]pyridine-8-carbonitrile (54.86 mg, 0.232 mmol, 1 equiv), K₃PO₄ (49.33 mg, 0.232 mmol, 1 equiv), and Pd(dppf)Cl₂ (18.93 mg, 0.023 mmol, 0.1 equiv) at room temperature under nitrogen atmosphere. The resulting mixture was stirred for 4 h at 80° C. under nitrogen atmosphere, then concentrated under reduced pressure to give a residue. The residue was purified by silica gel column chromatography, eluted with CH₂Cl₂/MeOH (20:1) to afford tert-butyl 4-(7-{8-cyano-2-methylimidazo[1,2-a]pyridin-6-yl}-5-fluoro-4-oxoquinazolin-3-yl)piperidine-1-carboxylate (60 mg, 51%) as an oil. LCMS (ES, m/z): 503 [M+H]⁺.

### Synthesis of Compound 588

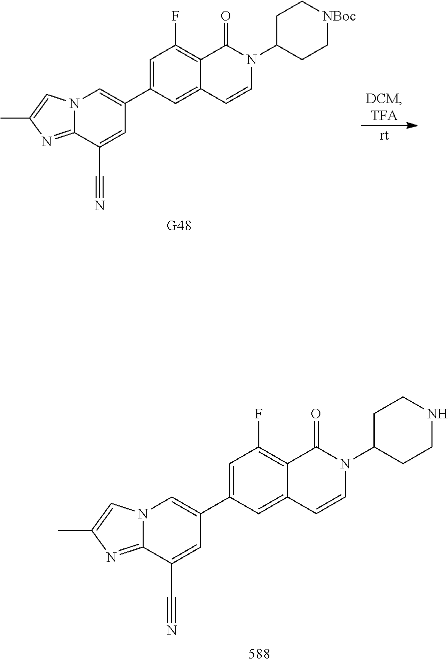 [SMILES: Cc1cn2cc(-c3cc(F)c4c(=O)n(C5CCN(C(=O)OC(C)(C)C)CC5)ccc4c3)cc(C#N)c2n1 ; Cc1cn2cc(-c3cc(F)c4c(=O)n(C5CCNCC5)ccc4c3)cc(C#N)c2n1]

A mixture of tert-butyl 4-(7-{8-cyano-2-methylimidazo[1,2-a]pyridin-6-yl}-5-fluoro-4-oxoquinazolin-3-yl)piperidine-1-carboxylate (60 mg, 0.119 mmol, 1 equiv), DCM (1 mL) and TFA (1 mL) was stirred for 2 h at room temperature, then concentrated under reduced pressure to give a residue. The residue was purified by reverse phase flash chromatography (Condition G7, Gradient 1) to afford 6-[5-fluoro-4-oxo-3-(piperidin-4-yl)quinazolin-7-yl]-2-methylimidazo[1,2-a]pyridine-8-carbonitrile (15 mg, 31%) as a solid. LCMS (ES, m/z): 403 [M+H]⁺. ¹H NMR (300 MHz, DMSO-d₆) δ 9.46 (d, J=1.8 Hz, 1H), 8.55-8.46 (m, 2H), 7.92 (dd, J=15.9, 1.4 Hz, 2H), 7.80 (dd, J=12.5, 1.8 Hz, 1H), 4.67 (s, 1H), 3.11 (d, J=11.8 Hz, 2H), 2.62 (t, J=11.6 Hz, 2H), 2.46-2.40 (m, 3H), 1.91 (d, J=9.8 Hz, 2H), 1.78 (d, J=12.1 Hz, 2H).

### Example G26: Synthesis of Compound 589

### Synthesis of Intermediate G49

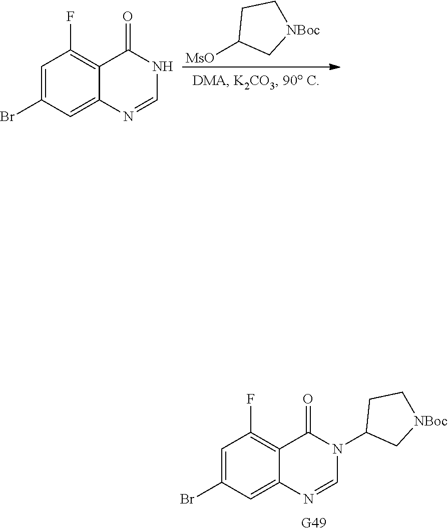 [SMILES: O=c1[nH]cnc2cc(Br)cc(F)c12 ; CC(C)(C)OC(=O)N1CCC(OS(C)(=O)=O)C1 ; CC(C)(C)OC(=O)N1CCC(n2cnc3cc(Br)cc(F)c3c2=O)C1]

A mixture of 7-bromo-5-fluoro-3H-quinazolin-4-one (1.0 g, 4.115 mmol, 1.0 equiv), DMA (10 mL), tert-butyl 3-(methanesulfonyloxy)pyrrolidine-1-carboxylate (1.31 g, 4.938 mmol, 1.2 equiv) and K₂CO₃ (1.71 g, 12.345 mmol, 3.0 equiv) was stirred overnight at 90° C. The resulting mixture was filtered, and the filter cake washed with DCM (3×20 mL). The filtrate was concentrated under reduced pressure to give a residue. The residue was purified by reverse flash chromatography (Condition G1, Gradient 1) to afford tert-butyl 3-(7-bromo-5-fluoro-4-oxoquinazolin-3-yl)pyrrolidine-1-carboxylate (380 mg, 24%) as a solid. LCMS (ES, m/z): 412 [M+H]⁺.

### Synthesis of Intermediate G50

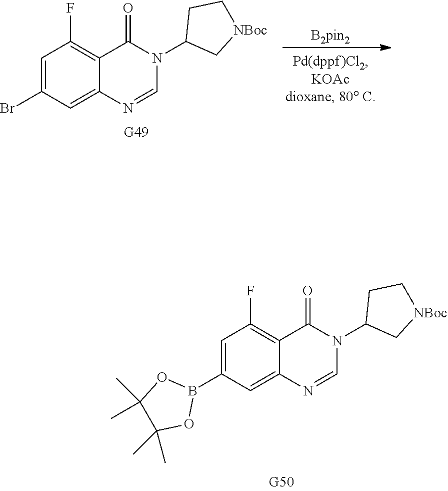 [SMILES: CC(C)(C)OC(=O)N1CCC(n2cnc3cc(Br)cc(F)c3c2=O)C1 ; CC(C)(C)OC(=O)N1CCC(n2cnc3cc(B4OC(C)(C)C(C)(C)O4)cc(F)c3c2=O)C1]

A mixture of tert-butyl 3-(7-bromo-5-fluoro-4-oxoquinazolin-3-yl)pyrrolidine-1-carboxylate (100 mg, 0.243 mmol, 1.0 equiv), 4,4,5,5-tetramethyl-2-(tetramethyl-1,3,2-dioxaborolan-2-yl)-1,3,2-dioxaborolane (73.92 mg, 0.292 mmol, 1.2 equiv), AcOK (71.42 mg, 0.729 mmol, 3.0 equiv), dioxane (2 mL) and Pd(dppf)Cl₂ (19.76 mg, 0.024 mmol, 0.1 equiv) was stirred for 4 h at 80° C. under nitrogen atmosphere. LCMS (ES, m/z): 460 [M+H]⁺.

### Synthesis of Intermediate G51

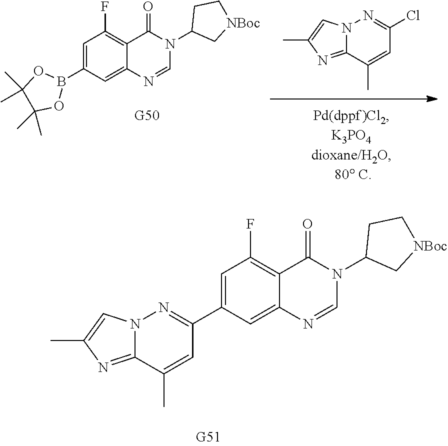 [SMILES: CC(C)(C)OC(=O)N1CCC(n2cnc3cc(B4OC(C)(C)C(C)(C)O4)cc(F)c3c2=O)C1 ; Cc1cn2nc(Cl)cc(C)c2n1 ; Cc1cn2nc(-c3cc(F)c4c(=O)n(C5CCN(C(=O)OC(C)(C)C)C5)cnc4c3)cc(C)c2n1]

To the stirred mixture of Intermediate G50 were added water (0.4 mL), 6-chloro-2,8-dimethylimidazo[1,2-b]pyridazine (43.49 mg, 0.239 mmol, 1.0 equiv), K₃PO₄ (50.83 mg, 0.239 mmol, 1.0 equiv) and Pd(dppf)Cl₂ (19.51 mg, 0.024 mmol, 0.1 equiv) under nitrogen atmosphere. The resulting mixture was stirred for 4 h at 80° C. under nitrogen atmosphere, then concentrated under reduced pressure to give a residue. The residue was purified by silica gel column chromatography, eluted with CH₂Cl₂/MeOH (20:1) to afford tert-butyl 3-(7-{2,8-dimethylimidazo[1,2-b]pyridazin-6-yl}-5-fluoro-4-oxoquinazolin-3-yl)pyrrolidine-1-carboxylate (100 mg, 87%) as a solid. LCMS (ES, m/z): 479 [M+H]⁺.

### Synthesis of Compound 589

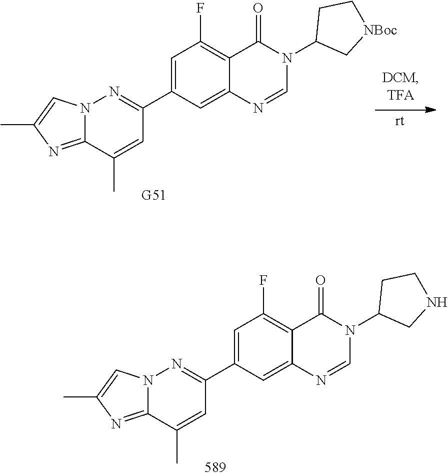 [SMILES: Cc1cn2nc(-c3cc(F)c4c(=O)n(C5CCN(C(=O)OC(C)(C)C)C5)cnc4c3)cc(C)c2n1 ; Cc1cn2nc(-c3cc(F)c4c(=O)n(C5CCNC5)cnc4c3)cc(C)c2n1]

A mixture of tert-butyl 3-(7-{2,8-dimethylimidazo[1,2-b]pyridazin-6-yl}-5-fluoro-4-oxoquinazolin-3-yl)pyrrolidine-1-carboxylate (60 mg, 0.125 mmol, 1 equiv), DCM (1 mL) and TFA (1 mL) was stirred for 2 h at room temperature, then concentrated under reduced pressure to give a residue. The residue was purified by Perp-HPLC (Condition G7, Gradient 1) to afford 7-{2,8-dimethylimidazo[1,2-b]pyridazin-6-yl}-5-fluoro-3-(pyrrolidin-3-yl)quinazolin-4-one (15 mg, 32%) as a solid. LCMS (ES, m/z): 379 [M+H]⁺. ¹H NMR (400 MHz, DMSO-d₆) δ 8.51 (d, J=5.0 Hz, 1H), 8.18-8.10 (m, 2H), 7.99-7.89 (m, 1H), 7.87 (s, 1H), 5.14 (dt, J=8.7, 4.5 Hz, 1H), 3.19-3.07 (m, 2H), 3.00 (dd, J=11.7, 3.9 Hz, 1H), 2.90-2.79 (m, 1H), 2.62 (s, 3H), 2.42 (s, 3H), 2.30-2.21 (m, 1H), 1.91 (q, J=7.3, 6.9 Hz, 1H).

G8, Gradient 2) to afford 6-(2,8-dimethylimidazo[1,2-b]pyridazin-6-yl)-8-fluoro-2-[(7S)-4-methyl-4-azaspiro[2.5]-octan-7-yl]phthalazin-1-one (21.7 mg, 42%) as a solid. LCMS: (ESI, m/z): 433.2 [M+H]⁺. ¹H NMR (400 MHz, METHANOL-d4) 6=8.58-8.51 (m, 2H), 8.40 (s, 2H), 8.34 (dd, J=1.2, 12.0 Hz, 1H), 5.40 (br s, 1H), 3.77 (br d, J=3.2 Hz, 1H), 3.57 (br dd, J=2.0, 11.2 Hz, 1H), 3.13 (s, 3H), 2.96 (br t, J=13.1 Hz, 1H), 2.84 (s, 3H), 2.74-2.63 (m, 4H), 2.16 (br d, J=15.0 Hz, 1H), 1.53-1.42 (m, 1H), 1.35 (br d, J=2.7 Hz, 1H), 1.26-1.09 (m, 2H), 1.00 (br dd, J=3.2, 6.4 Hz, 1H).

### Example G57: Synthesis of Compound 1540

### Synthesis of Compound 1540

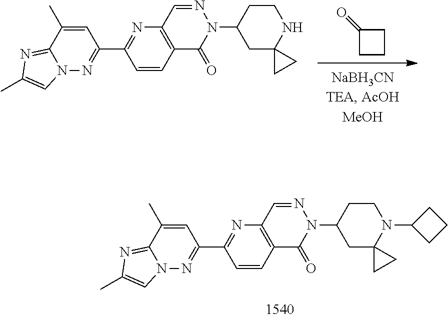 [SMILES: Cc1cn2nc(-c3ccc4c(=O)n(C5CCNC6(CC6)C5)ncc4n3)cc(C)c2n1 ; O=C1CCC1 ; Cc1cn2nc(-c3ccc4c(=O)n(C5CCN(C6CCC6)C6(CC6)C5)ncc4n3)cc(C)c2n1]

To a solution of 6-(4-azaspiro[2.5]octan-7-yl)-2-(2,8-dimethylimidazo[1,2-b]pyridazin-6-yl)pyrido[2,3-d]pyridazin-5-one (50.0 mg, 124 umol, 1.00 eq) in methanol (1.00 mL) was added triethylamine (25.2 mg, 249 umol, 2.00 eq). The reaction mixture was stirred at 25° C. for 10 min. To the reaction mixture was added acetic acid (29.9 mg, 498 umol, 28.4 uL, 4.00 eq) and formaldehyde (34.9 mg, 498 umol, 37.2 uL, 4.00 eq). The resulting mixture was stirred at 25° C. for an additional 30 min. To the reaction mixture was added sodium cyanoborohydride (39.9 mg, 635 umol, 5.10 eq). The resulting mixture was stirred at 30° C. for 24 h, diluted with water (10.0 mL), and extracted with dichloromethane (3×10.0 mL). The organic layers were combined, dried over Na₂SO₄, and filtered. The filtrate was concentrated under reduced pressure to give a solid. The solid was triturated with methanol. The resulting solid was collected by filtration and dried under vacuum to give 6-(4-cyclobutyl-4-azaspiro[2.5]octan-7-yl)-2-(2,8-dimethylimidazo[1,2-b]pyridazin-6-yl)pyrido[2,3-d]pyridazin-5-one (12.2 mg, 22%) as a solid. LCMS: (ESI, m/z): 456.2 [M+H]⁺. ¹H NMR (400 MHz, DMSO-d6) δ=8.85 (d, J=8.5 Hz, 1H), 8.75-8.62 (m, 2H), 8.42-8.30 (m, 2H), 5.34-5.19 (m, 1H), 4.51-4.41 (m, 1H), 3.61-3.50 (m, 1H), 3.40 (br d, J=11.8 Hz, 1H), 2.89-2.79 (m, 2H), 2.73 (s, 3H), 2.52 (br s, 3H), 2.30-2.25 (m, 2H), 2.18-1.99 (m, 3H), 1.83-1.69 (m, 2H), 1.40 (br dd, J=2.9, 14.3 Hz, 1H), 1.30-1.21 (m, 1H), 1.12 (br d, J=10.4 Hz, 1H), 0.98-0.79 (m, 2H).

### Example G58: Synthesis of Compound 1542

### Synthesis of Compound 1542

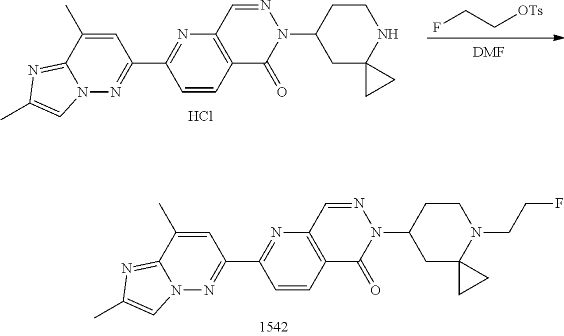

A mixture of 2-fluoroethyl 4-methylbenzenesulfonate (60.0 mg, 274 umol, 2.76 eq), 6-(4-azaspiro[2.5]octan-7-yl)-2-(2,8-dimethylimidazo[1,2-b]pyridazin-6-yl)pyrido[2,3-d]pyridazin-5-one (40 mg, 99.6 umol, 1.00 eq), and potassium carbonate (68.8 mg, 498 umol, 5.00 eq) in dimethyl formamide (0.800 mL) was stirred at 100° C. for 2 h. The resulting mixture was filtered and the filtrate concentrated under vacuum to give an oil. The oil was purified by prep-HPLC (Condition G1, Gradient 5) to afford the 2-(2,8-dimethylimidazo[1,2-b]pyridazin-6-yl)-6-[4-(2-fluoroethyl)-4-azaspiro[2.5]octan-7-1]pyrido[2,3-d]pyridazin-5-one (10.0 mg, 22%). LCMS: (ESI, m/z): 448.3 [M+H]⁺. ¹H NMR (400 MHz, CD₃OD) δ=8.78-8.67 (m, 2H), 8.59 (s, 1H), 8.18 (d, J=1.1 Hz, 1H), 7.97 (d, J=0.7 Hz, 1H), 5.28 (tt, J=4.3, 11.8 Hz, 1H), 4.61-4.54 (m, 1H), 4.48-4.42 (m, 1H), 3.29-2.95 (m, 4H), 2.74-2.62 (m, 4H), 2.50 (s, 3H), 2.47-2.35 (m, 1H), 1.61 (br dd, J=2.1, 13.0 Hz, 1H), 1.08-0.99 (m, 1H), 0.81-0.73 (m, 2H), 0.65-0.48 (m, 2H).

### Example G59: Synthesis of Compound 1543

### Synthesis of Compound 1543

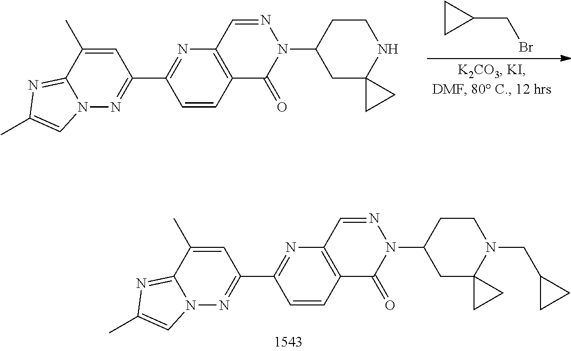

A mixture of bromomethylcyclopropane (77.0 mg, 570 umol, 4.58 eq), 6-(4-azaspiro[2.5]octan-7-yl)-2-(2,8-dimethylimidazo[1,2-b]pyridazin-6-yl)pyrido[2,3-d]pyridazin-5-one (50.0 mg, 125 umol, 1.00 eq), potassium iodide (41.5 mg, 294 umol, 2.00 eq), and potassium carbonate (86.0 mg, 622 umol, 5.00 eq) in dimethyl formamide (0.800 mL) was stirred at 80° C. for 12 h. The resulting mixture was diluted with water (50.0 mL) and extracted with ethyl acetate (3×30.0 mL). The organic phase was washed with brine (10.0 mL), dried over Na₂SO₄, and filtered. The filtrate was concentrated under reduced pressure to give a solid. The solid was triturated with methyl tert-butyl ether at 25° C. for 10 min. The resulting solid was collected by filtration and dried under vacuum to give 6-[4-(cyclopropylmethyl)-4-azaspiro[2.5]octan-7-yl]-2-(2,8-dimethylimidazo[1,2-b]pyridazin-6-yl)pyrido[2,3-d]pyridazin-5-one (15.0 mg, 30%) as a solid. LCMS: (ESI, m/z): 456.3 [M+H]⁺. ¹H NMR (400 MHz, METHANOL-d4) 6=8.75 (d, J=3.9 Hz, 2H), 8.61 (s, 1H), 8.21 (d, J=1.1 Hz, 1H), 7.99 (d, J=0.7 Hz, 1H), 5.28 (tt, J=4.4, 11.8 Hz, 1H), 3.47-3.39 (m, 1H), 3.19-3.07 (m, 1H), 2.82-2.64 (m, 6H), 2.51 (s, 3H), 2.38 (br dd, J=4.0, 12.3 Hz, 1H), 1.61 (br dd, J=2.0, 13.1 Hz, 1H), 1.03-0.73 (m, 4H), 0.66-0.47 (m, 4H), 0.30-0.15 (m, 2H)

### Example G60: Synthesis of Compound 1547-1549

### Synthesis of Intermediate G95

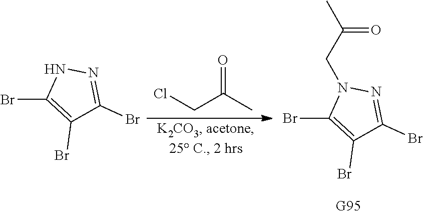

To a mixture of 3,4,5-tribromo-1H-pyrazole (4.80 g, 15.7 mmol, 1.00 eq) and K₂CO₃ (6.53 g, 47.2 mmol, 3.00 eq) in acetone (62.2 mL) was added 1-chloropropan-2-one (1.43 g, 15.4 mmol, 0.98 eq). The reaction mixture was stirred at 25° C. for 2 h, then diluted with water (200 mL), and extracted with dichloromethane (200 mL×3). The organic layers were combined, dried over Na₂SO₄, and filtered. The filtrate was concentrated under reduced pressure to give an oil. The oil was triturated with tert-butyl methyl ether (200 mL) at 25° C. for 40 min. The resulting solid was collected by filtration and dried under vacuum to give tribromopyrazole-1-yl)propan-2-one (42.0 g, 72%) as a solid. LCMS: (ESI, m/z): 362.8 [M+H]⁺. ¹H NMR (400 MHz, CHLOROFORM-d) δ=4.98 (s, 2H), 2.20 (s, 3H).

### Synthesis of Intermediate G96

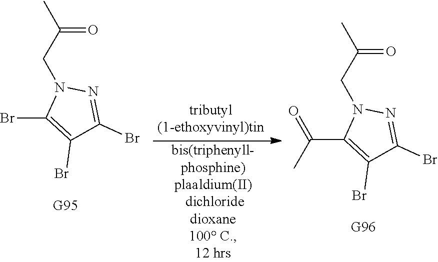

A mixture of 1-(3,4,5-tribromopyrazole-1-yl)propan-2-one (A15-3, 32.0 g, 88.6 mmol, 1.00 eq), tributyl(1-ethoxyvinyl)stannane (28.5 g, 79.1 mmol, 26.7 mL, 0.89 eq) and bis(triphenylphosphine) palladium(ii) dichloride (3.11 g, 4.43 mmol, 0.050 eq) in dioxane (500 mL) was stirred at 100° C. for 12 h, then diluted with water (300 mL) and ethyl acetate (200 mL×2). The organic layers were combined, washed with brine (500 mL×2), dried over anhydrous Na₂SO₄, and filtered. The filtrate was concentrated under reduced pressure to give an oil. The oil was purified by column chromatography on silica gel eluted with petroleum ether/ethyl acetate (I/O to 5/1) to give 1-(5-acetyl-3,4-dibromo-pyrazol-1-yl)propan-2-one (7.00 g, 24%) as a solid. LCMS: (ESI, m/z): 342.2 [M+18]⁺. ¹H NMR (400 MHz, METHANOL-d4) 6=5.33 (s, 2H), 2.67 (s, 3H), 2.22 (s, 3H).

### Synthesis of Intermediate G97

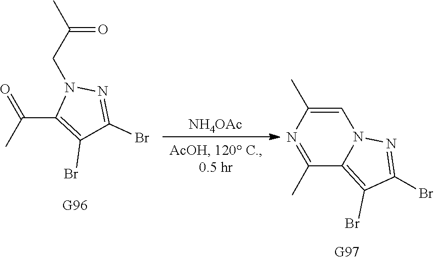

To a solution of 1-(5-acetyl-3,4-dibromo-pyrazol-1-yl)propan-2-one (6.00 g, 18.5 mmol, 1.00 eq) in acetic acid (18.0 mL) was added ammonium acetate (14.2 g, 185 mmol, 10.0 eq) under N₂ atmosphere. The resulting mixture was stirred at 120° C. for 0.5 hr, then partitioned between water (50.0 mL) and dichloromethane (50.0 mL). The organic phase was separated, washed with water (30.0 mL×3), dried over Na₂SO₄, and filtered. The filtrate was concentrated under reduced pressure to give residue. The residue was triturated with petroleum ether/ethyl acetate (10/1, 20.0 mL) at 25° C. for 30 min. The resulting solid was collected by filtration and dried under vacuum to give 2,3-dibromo-4,6-dimethyl-pyrazolo[1,5-a]pyrazine (5.00 g, 89%) as a solid. LCMS: (ESI, m/z): 305.9 [M+H]⁺. ¹H NMR (400 MHz, DMSO-d6) δ ppm 8.53 (s, 1H) 2.85 (s, 3H) 2.39 (s, 3H).

### Synthesis of Intermediate G98

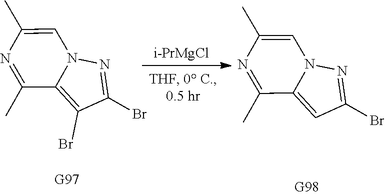

To a solution of 2,3-dibromo-4,6-dimethyl-pyrazolo[1,5-a]pyrazine (5.00 g, 16.4 mmol, 1.00 eq) in tetrahydrofuran (125 mL) was added i-PrMgCl (2.00 M, 8.20 mL, 1.00 eq) at 0° C. The reaction mixture was stirred at 0° C. for 0.5 h, then quenched with methanol (5.00 mL). The resulting mixture was partitioned between ethyl acetate (100 mL) and water (50.0 mL). The organic phase was washed with brine (30.0 mL×3), dried over Na₂SO₄, and filtered. The filtrate was concentrated under reduced pressure to give residue. The residue was triturated with petroleum ether (5.00 mL) at 25° C. for 10 min. The resulting solid was collected by filtration and dried under vacuum to give 2-bromo-4,6-dimethyl-pyrazolo[1,5-a]pyrazine (4.00 g, 66%) as a solid. LCMS: (ESI, m/z): 226.2 [M+H]⁺. ¹H NMR (400 MHz, DMSO-d6) δ ppm 8.45 (s, 1H) 7.15 (d, J=0.75 Hz, 1H) 2.63 (s, 3H) 2.39 (s, 3H).

### Synthesis of Intermediate G99

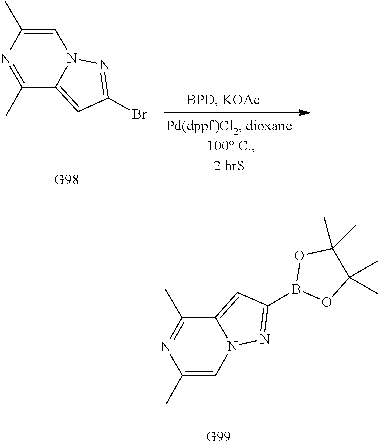

To a mixture of 2-bromo-4,6-dimethyl-pyrazolo[1,5-a]pyrazine (500 mg, 2.21 mmol, 1.00 eq) and bis(pinacolato)diboron (1.12 g, 4.42 mmol, 2.00 eq) in 1,4-dioxane (5.00 mL) were added bis(dibenzylideneacetone)palladium (190 mg, 331 umol, 0.15 eq), 2-(dicyclohexylphosphanyl)-2,4,6-tris(isopropyl)biphenyl (316 mg, 663 umol, 0.30 eq) and potassium acetate (325 mg, 3.32 mmol, 1.50 eq) under N₂ atmosphere. The reaction mixture was stirred at 100° C. for 2 h under N₂ atmosphere. The resulting mixture was filtered and the filtrate concentrated under reduced pressure to give (4,6-dimethylpyrazolo[1,5-a]pyrazin-2-yl)boronic acid (422 mg) as an oil. LCMS: (ESI, m/z): 274.7 [M−84]⁺.

### Synthesis of Intermediate G100

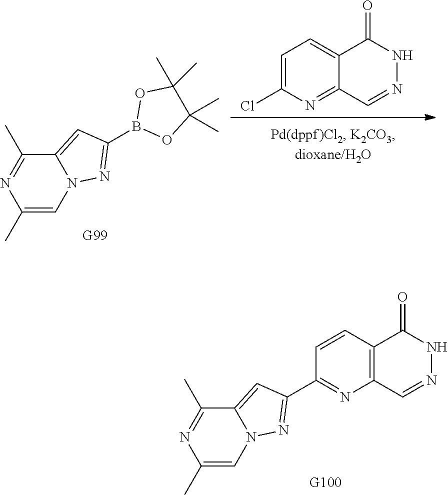

To a solution of (4,6-dimethylpyrazolo[1,5-a]pyrazin-2-yl)boronic acid (422 mg, 2.21 mmol, 1.00 eq) in 1,4-dioxane (4.00 mL) were added 2-chloro-6H-pyrido[2,3-d]pyridazin-5-one (401 mg, 2.21 mmol, 1.00 eq), a solution of K₂CO₃ (916 mg, 6.63 mmol, 3.00 eq) in water (1.00 mL), and 1,1′-bis(diphenylphosphino)ferrocenedichloro palladium (II) (161 mg, 221 umol, 0.100 eq) under N₂ atmosphere. The reaction mixture was stirred at 100° C. for 1 h, then poured into water (10.0 mL) and filtered. The filter cake was triturated with ethyl acetate/methanol (4/1, 10.0 mL) at 25° C. for 10 min. The resulting solid was collected by filtration and dried under vacuum to give 2-(4,6-dimethylpyrazolo[1,5-a]pyrazin-2-yl)-6H-pyrido[2,3-d]pyridazin-5-one (465 mg, 72%) as a solid. LCMS: (ESI, m/z): 293.2 [M+H]⁺.

### Synthesis of Intermediate G101

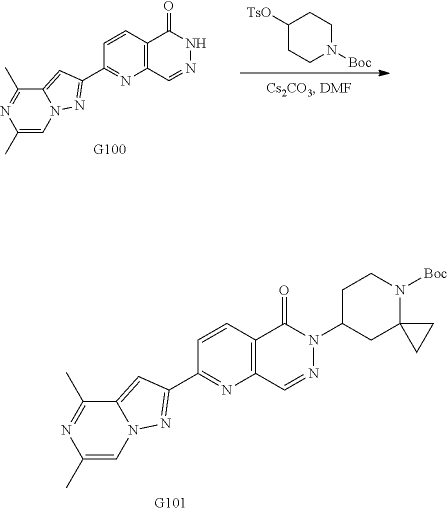 [SMILES: Cc1cn2nc(-c3ccc4c(=O)[nH]ncc4n3)cc2c(C)n1 ; Cc1ccc(S(=O)(=O)OC2CCN(C(=O)OC(C)(C)C)CC2)cc1 ; Cc1cn2nc(-c3ccc4c(=O)n(C5CCN(C(=O)OC(C)(C)C)C6(CC6)C5)ncc4n3)cc2c(C)n1]

A mixture of 2-(4,6-dimethylpyrazolo[1,5-a]pyrazin-2-yl)-6H-pyrido[2,3-d]pyridazin-5-one (465 mg, 1.59 mmol, 1.00 eq), tert-butyl 7-(p-tolylsulfonyloxy)-4-azaspiro[2.5]octane-4-carboxylate (606 mg, 1.59 mmol, 1.00 eq), and Cs₂CO₃ (1.55 g, 4.77 mmol, 3.00 eq) in N,N-dimethylformamide (12.0 mL) was stirred at 100° C. for 12 h. The reaction mixture was poured into water (50.0 mL) and extracted with ethyl acetate (50.0 mL×3). The organic layer was combined, dried over Na₂SO₄, and filtered. The filtrate was concentrated under reduced pressure to give a residue. The residue was purified by column chromatography (SiO₂, petroleum ether/ethyl acetate=1/0 to 2/3) to give tert-butyl 7-[2-(4,6-dimethylpyrazolo[1,5-a]pyrazin-2-yl)-5-oxo-pyrido[2,3-d]pyridazin-6-yl]-4-azaspiro[2.5]octane-4-carboxylate (250 mg, 31%) as a solid. LCMS: (ESI, m/z): 502.3 [M+H]⁺.

### Synthesis of Compound 1547

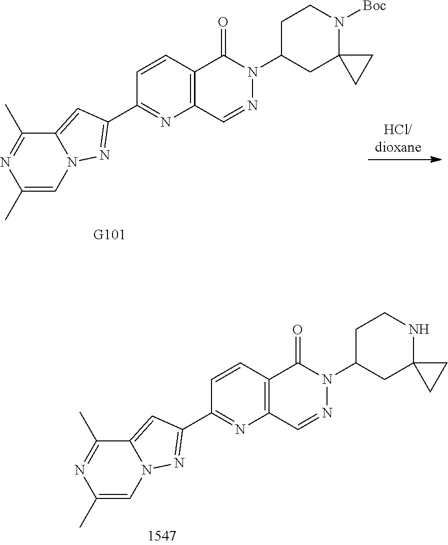 [SMILES: Cc1cn2nc(-c3ccc4c(=O)n(C5CCN(C(=O)OC(C)(C)C)C6(CC6)C5)ncc4n3)cc2c(C)n1 ; Cc1cn2nc(-c3ccc4c(=O)n(C5CCNC6(CC6)C5)ncc4n3)cc2c(C)n1]

To a solution of tert-butyl 7-[2-(4,6-dimethylpyrazolo[1,5-a]pyrazin-2-yl)-5-oxo-pyrido[2,3-d]pyridazin-6-yl]-4-azaspiro[2.5]octane-4-carboxylate (200 mg, 398 umol, 1.00 eq) in 1,4-dioxane (0.200 mL) was added HCl/dioxane (4.00 M, 2.00 mL, 20.0 eq). The reaction mixture was stirred at 25° C. for 0.5 h, then filtered, and the filter cake purified by prep-HPLC (Condition G8, Gradient 2) to give 6-(4-azaspiro[2.5]octan-7-yl)-2-(4,6-dimethylpyrazolo[1,5-a]pyrazin-2-yl)pyrido[2,3-d]pyridazin-5-one (130 mg, 323 umol, 65%) as a solid. LCMS: (ESI, m/z): 402.1 [M+H]⁺. ¹H NMR (400 MHz, METHANOL-d4) δ ppm 8.77 (d, J=8.51 Hz, 1H) 8.57-8.63 (m, 2H) 8.49 (s, 1H) 7.85 (d, J=0.63 Hz, 1H) 5.40 (tt, J=11.60, 3.97 Hz, 1H) 3.59-3.67 (m, 1H) 3.42 (td, J=13.01, 3.25 Hz, 1H) 2.86 (s, 3H) 2.80 (t, J=12.69 Hz, 1H) 2.54 (d, J=0.63 Hz, 3H) 2.37-2.49 (m, 1H) 2.23-2.32 (m, 1H) 1.61-1.70 (m, 1H) 1.09-1.19 (m, 2H) 1.01-1.07 (m, 1H) 0.94-1.01 (m, 1H).

### Synthesis of Compounds 1548 and 1549

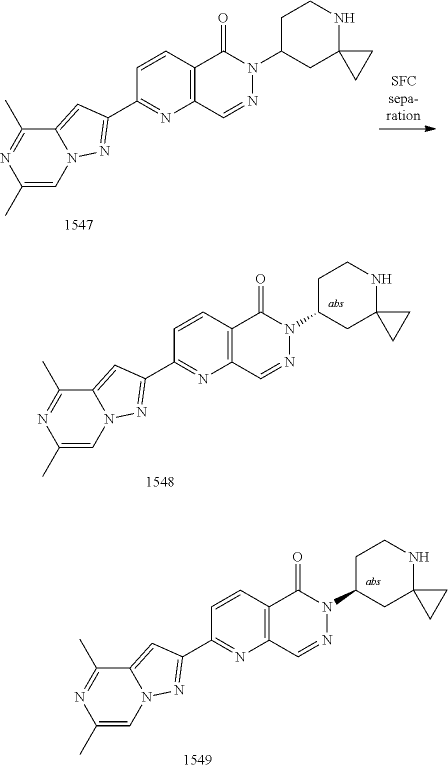 [SMILES: Cc1cn2nc(-c3ccc4c(=O)n(C5CCNC6(CC6)C5)ncc4n3)cc2c(C)n1 ; Cc1cn2nc(-c3ccc4c(=O)n([C@@H]5CCNC6(CC6)C5)ncc4n3)cc2c(C)n1 ; Cc1cn2nc(-c3ccc4c(=O)n([C@H]5CCNC6(CC6)C5)ncc4n3)cc2c(C)n1]

Compound 1547 (100 mg, 249 umol) was purified by SFC (Condition G2) to give 6-[(7R)-4-azaspiro[2.5]octan-7-yl]-2-(4,6-dimethylpyrazolo[1,5-a]pyrazin-2-yl)pyrido[2,3-d]pyridazin-5-one (1548, 30.0 mg, 30%) and 6-[(7S)-4-azaspiro[2.5]octan-7-yl]-2-(4,6-dimethylpyrazolo[1,5-a]pyrazin-2-yl)pyrido[2,3-d]pyridazin-5-one (1549, 30.0 mg, 30%) were obtained as solids. 1548: LCMS: (ESI, m/z): 402.1 [M+H]⁺. ¹H NMR (400 MHz, METHANOL-d4) δ ppm 8.73 (d, J=8.38 Hz, 1H) 8.52-8.58 (m, 2H) 8.36 (s, 1H) 7.70 (s, 1H) 5.23-5.36 (m, 1H) 3.26 (br d, J=14.01 Hz, 1H) 3.03 (td, J=13.07, 2.75 Hz, 1H) 2.78-2.81 (m, 3H) 2.58 (t, J=12.26 Hz, 1H) 2.51 (s, 3H) 2.17 (qd, J=12.51, 4.25 Hz, 1H) 1.98-2.06 (m, 1H) 1.34-1.42 (m, 1H) 0.73-0.86 (m, 2H) 0.62-0.70 (m, 2H). 1549: LCMS: (ESI, m/z): 402.1 [M+H]⁺. ¹H NMR (400 MHz, METHANOL-d4) δ ppm 8.74 (d, J=8.50 Hz, 1H) 8.54-8.59 (m, 2H) 8.38 (s, 1H) 7.72 (s, 1H) 5.27 (tt, J=11.52, 4.11 Hz, 1H) 3.12-3.18 (m, 1H) 2.88-2.98 (m, 1H) 2.80 (s, 3H) 2.46-2.57 (m, 4H) 2.04-2.17 (m, 1H) 1.89-1.99 (m, 1H) 1.25-1.34 (m, 1H) 0.64-0.75 (m, 2H) 0.51-0.61 (m, 2H).

### Example G61: Synthesis of Compound 1552

### Synthesis of Intermediate G102

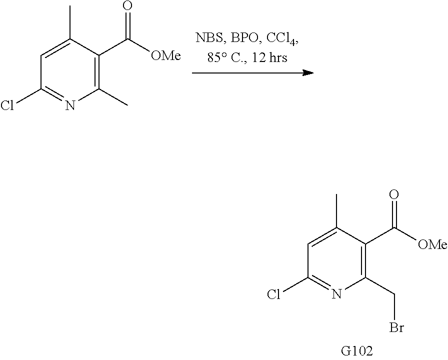

To a solution of methyl 6-chloro-2,4-dimethyl-pyridine-3-carboxylate (2.70 g, 13.5 mmol, 1.00 eq) in CCl₄ (27.0 mL) were added N-bromosuccinimide (2.53 g, 14.2 mmol, 1.05 eq) and benzoyl peroxide (873 mg, 2.70 mmol, 0.200 eq). The reaction mixture was stirred at 85° C. for 12 h, then diluted with water (100 mL) and extracted with ethyl acetate (100 mL×3). The organic layers were combined, dried over Na₂SO₄, and filtered. The filtrate was concentrated under reduced pressure to give residue. The residue was purified by prep-HPLC (Condition G9, Gradient 1) to give methyl 2-(bromomethyl)-6-chloro-4-methyl-pyridine-3-carboxylate (1.00 g, 27%) as a solid. LCMS: (ESI, m/z): 280.1 [M+H]⁺. ¹H NMR (400 MHz, DMSO-d6) δ ppm 7.56 (s, 1H) 4.63 (s, 2H) 3.92 (s, 3H) 2.34 (s, 3H).

### Synthesis of Intermediate G103

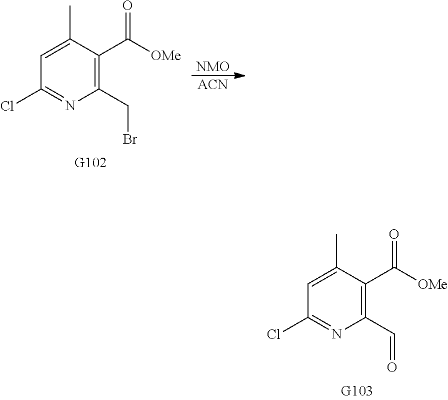

To a mixture of N-methylmorpholine oxide (546 mg, 4.67 mmol, 2.0 eq) and NMO (800 mg, 2.33 mmol, 1.00 eq) in acetonitrile (4.00 mL) was added a solution of methyl 2-(bromomethyl)-6-chloro-4-methyl-pyridine-3-carboxylate (650 mg, 2.33 mmol, 1.00 eq) in acetonitrile (1.00 mL) at 0° C. under N₂ atmosphere. The reaction mixture was stirred at 25° C. for 5 h to afford a solution of methyl 6-chloro-2-formyl-4-methyl-pyridine-3-carboxylate in acetonitrile. LCMS: (ESI, m/z): 214.5 [M+H]⁺.

### Synthesis of Intermediate G104

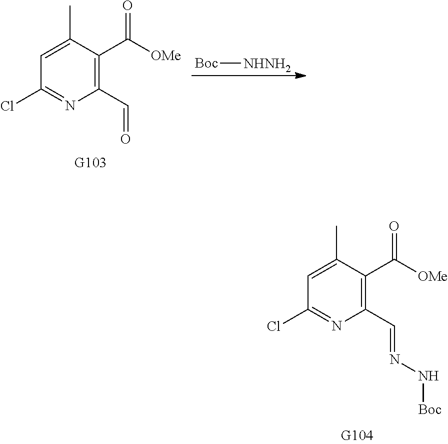 [SMILES: COC(=O)c1c(C)cc(Cl)nc1C=O ; CC(C)(C)OC(=O)NN ; COC(=O)c1c(C)cc(Cl)nc1/C=N/NC(=O)OC(C)(C)C]

To the solution of methyl 6-chloro-2-formyl-4-methyl-pyridine-3-carboxylate (490 mg, 2.29 mmol, 1.00 eq) in acetonitrile (1.50 mL) was added tert-butylcarbazate (606 mg, 4.59 mmol, 2.00 eq). The reaction mixture was stirred at 25° C. for 12 h, then filtered, and the filtrate concentrated under reduced pressure to give a residue. The residue was purified by column chromatography (SiO₂, petroleum ether/ethyl acetate=I/O to 3/1) to give methyl 2-[(E)-(tert-butoxycarbonylhydrazino)methyl]-6-chloro-4-methyl-pyridine-3-carboxylate (41-3A, 300 mg, 40%) as a solid. LCMS: (ESI, m/z): 328.3 [M+H]⁺. ¹H NMR (400 MHz, DMSO-d₆) δ ppm 11.17-11.38 (m, 1H) 7.91 (s, 1H) 7.51 (s, 1H) 3.89 (s, 3H) 2.26 (s, 3H) 1.47 (s, 9H).

### Synthesis of Intermediate G105

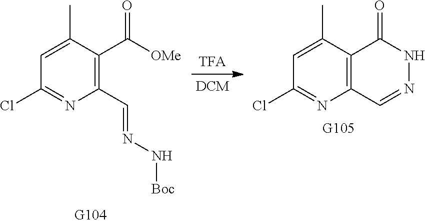 [SMILES: COC(=O)c1c(C)cc(Cl)nc1/C=N/NC(=O)OC(C)(C)C ; Cc1cc(Cl)nc2cn[nH]c(=O)c12]

To a solution of methyl 2-[(E)-(tert-butoxycarbonylhydrazino)methyl]-6-chloro-4-methyl-pyridine-3-carboxylate (250 mg, 762 umol, 1.00 eq) in dichloromethane (3.00 mL) was added trifluoroacetic acid (869 mg, 7.63 mmol, 564 μL, 10.0 eq). The reaction mixture was stirred at 25° C. for 2 h, then diluted with dichloromethane (5.00 mL), washed with saturated aqueous NaHCO₃ (3.00 mL×3), dried over Na₂SO₄, and filtered. The filtrate was concentrated under reduced pressure to give 2-chloro-4-methyl-6H-pyrido[2,3-d]pyridazin-5-one (140 mg, 94%) as a solid. LCMS: (ESI, m/z): 196.1 [M+H]⁺. ¹H NMR (400 MHz, DMSO-d6) δ ppm 12.90 (br s, 1H) 8.27 (s, 1H) 7.77 (s, 1H) 2.82 (s, 3H).

### Synthesis of Intermediate G106

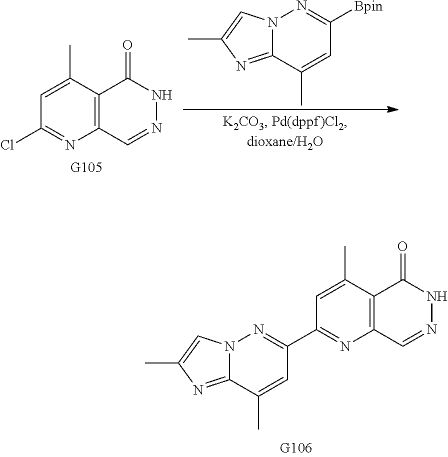

To a mixture of 2-chloro-4-methyl-6H-pyrido[2,3-d]pyridazin-5-one (130 mg, 664 umol, 1.00 eq) in 1,4-dioxane (4.00 mL) and water (1.00 mL) were added 2,8-dimethyl-6-(4,4,5,5-tetramethyl-1,3,2-dioxaborolan-2-yl)imidazo[1,2-b]pyridazine (363 mg, 1.33 mmol, 2.00 eq), K₂CO₃ (275 mg, 1.99 mmol, 3.00 eq), and 1,1′-bis(diphenylphosphino)ferrocenedichloro palladium(II) (48.6 mg, 66.4 umol, 0.100 eq) under N₂ atmosphere. The reaction mixture was stirred at 80° C. for 1 h, then poured into water (2.00 mL) and filtered. The filtered cake was triturated with methanol (3.00 mL). The resulting solid was collected by filtration and dried under vacuum to give 2-(2,8-dimethylimidazo[1,2-b]pyridazin-6-yl)-4-methyl-6H-pyrido[2,3-d]pyridazin-5-one (150 mg, 74%) as a solid. LCMS: (ESI, m/z): 307.5 [M+H]⁺. ¹H NMR (400 MHz, DMSO-d6) δ ppm 12.57-13.14 (m, 1H) 8.42 (s, 1H) 8.36 (s, 1H) 8.15 (s, 1H) 8.04 (d, J=0.88 Hz, 1H) 2.94 (s, 3H) 2.66 (s, 3H) 2.44 (s, 3H).

### Synthesis of Intermediate G107

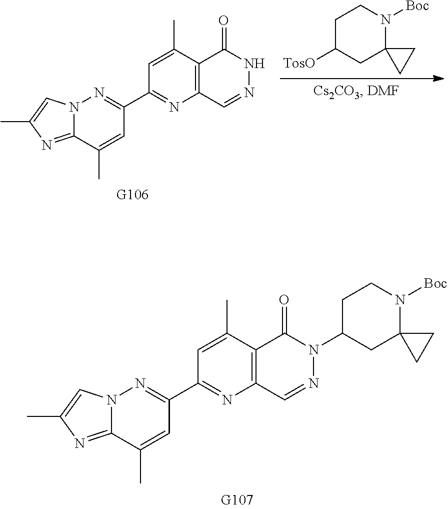

A mixture of 2-(2,8-dimethylimidazo[1,2-b]pyridazin-6-yl)-4-methyl-6H-pyrido[2,3-d]pyridazin-5-one (50.0 mg, 163 umol, 1.00 eq), tert-butyl 7-(p-tolylsulfonyloxy)-4-azaspiro[2.5]octane-4-carboxylate (68.5 mg, 179 umol, 1.10 eq), and Cs₂CO₃ (159 mg, 489 umol, 3.00 eq) in N,N-dimethylformamide (1.00 mL) was stirred at 80° C. for 12 h. The reaction mixture was poured into water (5.00 mL) and extracted with ethyl acetate (5×5.00 mL). The organic layers were combined, dried over Na₂SO₄, and filtered. The filtrate was concentrated under reduced pressure to give a residue. The residue was purified by column chromatography (SiO₂, petroleum ether/ethyl acetate=1/1) to give tert-butyl 7-[2-(2,8-dimethylimidazo[1,2-b]pyridazin-6-yl)-4-methyl-5-oxo-pyrido[2,3-d]pyridazin-6-yl]-4-azaspiro[2.5]octane-4-carboxylate (19.0 mg, 11%) as a solid. LCMS: (ESI, m/z): 516.4 [M+H]⁺. ¹H NMR (400 MHz, METHANOL-d4) δ ppm 8.48 (d, J=7.50 Hz, 2H) 8.18 (s, 1H) 7.98 (s, 1H) 5.30-5.43 (m, 1H) 3.84-3.96 (m, 2H) 3.00 (s, 3H) 2.70 (s, 3H) 2.51 (s, 3H) 1.84-1.93 (m, 2H) 1.65-1.79 (m, 2H) 1.46 (s, 9H) 1.09-1.15 (m, 1H) 0.86-1.00 (m, 3H).

### Synthesis of Compound 1552

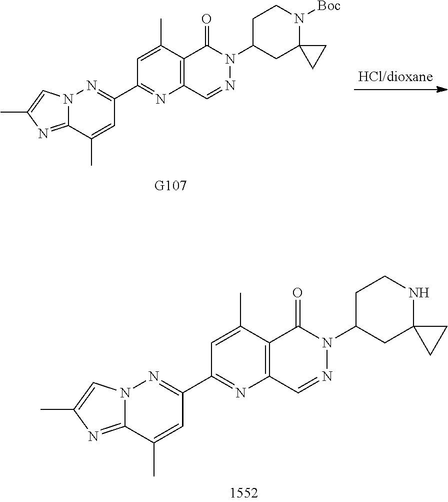 [SMILES: Cc1cn2nc(-c3cc(C)c4c(=O)n(C5CCN(C(=O)OC(C)(C)C)C6(CC6)C5)ncc4n3)cc(C)c2n1 ; Cc1cn2nc(-c3cc(C)c4c(=O)n(C5CCNC6(CC6)C5)ncc4n3)cc(C)c2n1]

To a solution of tert-butyl 7-[2-(2,8-dimethylimidazo[1,2-b]pyridazin-6-yl)-4-methyl-5-oxo-pyrido[2,3-d]pyridazin-6-yl]-4-azaspiro[2.5]octane-4-carboxylate (18.0 mg, 34.9 umol, 1.00 eq) in 1,4-dioxane (0.100 mL) was added HCl/dioxane (4.00 M, 0.100 mL, 11.4 eq). The reaction mixture was stirred at 25° C. for 2 h, then concentrated under reduced pressure to give residue. The residue was purified by prep-HPLC (Condition G8, Gradient 1) to give 6-(4-azaspiro[2.5]octan-7-yl)-2-(2,8-dimethylimidazo[1,2-b]pyridazin-6-yl)-4-methyl-pyrido[2,3-d]pyridazin-5-one (9.06 mg, 62%) as a solid. LCMS: (ESI, m/z): 416.2 [M+H]⁺. ¹H NMR (400 MHz, METHANOL-d4) δ ppm 8.66 (d, J=0.88 Hz, 1H) 8.60 (d, J=0.75 Hz, 1H) 8.57 (s, 1H) 8.32 (d, J=0.75 Hz, 1H) 5.37 (tt, J=11.55, 3.96 Hz, 1H) 3.59-3.66 (m, 1H) 3.39-3.44 (m, 1H) 3.06 (s, 3H) 2.82 (d, J=0.88 Hz, 3H) 2.77 (br t, J=12.82 Hz, 1H) 2.65 (d, J=0.63 Hz, 3H) 2.35-2.48 (m, 1H) 2.21-2.32 (m, 1H) 1.61-1.69 (m, 1H) 1.09-1.20 (m, 2H) 1.00-1.07 (m, 1H) 0.93-1.00 (m, 1H).

### Example G62: Synthesis of Compound 351

### Synthesis of Intermediate G108

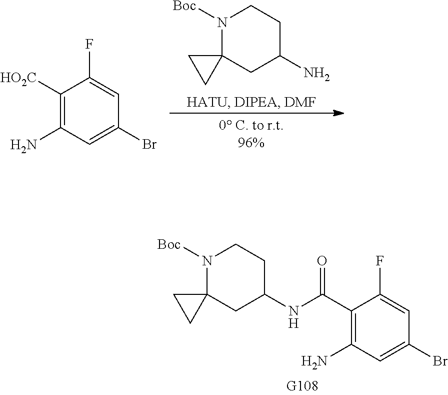

A mixture of 2-amino-4-bromo-6-fluorobenzoic acid (900 mg, 3.85 mmol) and tert-butyl 7-amino-4-azaspiro[2.5]octane-4-carboxylate (1.0 g, 4.23 mmol) in DMF (19 mL) and cooled to 0° C. in an ice-water bath. To the reaction mixture was added DIPEA (2.0 mL, 11.5 mmol) dropwise, followed by HATU (1.6 g, 4.23 mmol). The resulting mixture was warmed to room temperature over 2 h, then diluted with ethyl acetate (40 mL), washed with saturated aqueous NH₄Cl (40 mL), followed by saturated NaHCO₃ (40 mL), then brine (40 mL). The organic layer was dried over anhydrous Na₂SO₄, filtered, and the filtrate concentrated in vacuo to afford tert-butyl 7-(2-amino-4-bromo-6-fluorobenzamido)-4-azaspiro[2.5]octane-4-carboxylate (1.64 g, 96%) as a solid. LCMS (ES, m/z): 464.1 [M+Na]⁺.

### Synthesis of Intermediate G109

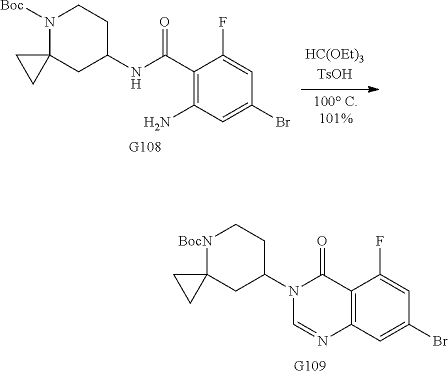 [SMILES: CC(C)(C)OC(=O)N1CCC(NC(=O)c2c(N)cc(Br)cc2F)CC12CC2 ; CC(C)(C)OC(=O)N1CCC(n2cnc3cc(Br)cc(F)c3c2=O)CC12CC2]

A mixture of 7-(2-amino-4-bromo-6-fluorobenzamido)-4-azaspiro[2.5]octane-4-carboxylate (1.64 g, 3.85 mmol) and p-toluenesulfonic acid monohydrate (344 mg, 1.81 mmol) in triethyl orthoformate (18.4 mL, 109 mmol) was heated at 100° C. for 72 hours. The resulting mixture was cooled to room temperature, then concentrated under reduced pressure to give a residue. The residue was dissolved in ethyl acetate (50 mL) and washed with saturated NaHCO₃ (40 mL), followed by brine (2×30 mL). The organic phase was dried over Na₂SO₄, filtered, and concentrated in vacuo to afford tert-butyl 7-(7-bromo-5-fluoro-4-oxoquinazolin-3(4H)-yl)-4-azaspiro[2.5]octane-4-carboxylate (1.66 g) as a solid. LCMS (ES, m/z): 396.0 [MH-Bu]⁺.

### Synthesis of Intermediate G10

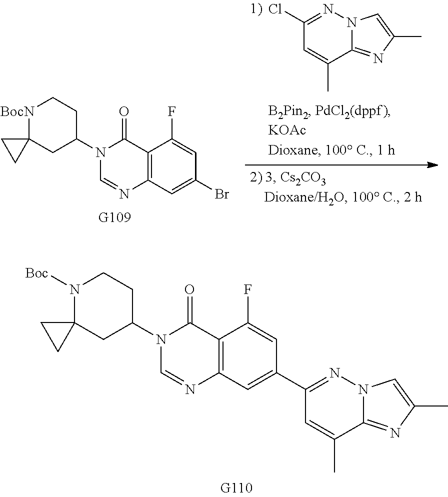 [SMILES: Cc1cn2nc(Cl)cc(C)c2n1 ; CC(C)(C)OC(=O)N1CCC(n2cnc3cc(Br)cc(F)c3c2=O)CC12CC2 ; Cc1cn2nc(-c3cc(F)c4c(=O)n(C5CCN(C(=O)OC(C)(C)C)C6(CC6)C5)cnc4c3)cc(C)c2n1]

A mixture of 6-chloro-2,8-dimethylimidazo[1,2-b]pyridazine (1.51 g, 8.32 mmol), B₂Pin₂ (2.21 g, 8.62 mmol), Pd(dppf)Cl₂·DCM (485 mg, 663 umol), and KOAc (1.97 g, 19.9 mmol) in dioxane (33 mL) was heated to 100° C. for 1 h. To the reaction mixture were added a solution of tert-butyl 7-(7-bromo-5-fluoro-4-oxoquinazolin-3(4H)-yl)-4-azaspiro[2.5]octane-4-carboxylate (1.5 g, 3.32 mmol) in dioxane (28 mL), followed by Cs₂CO₃ (3.24 g, 9.95 mmol), then water (5.5 mL) under argon. The reaction mixture was heated at 100° C. for 2 h, then cooled to room temperature. The resulting mixture was filtered over celite using 20% MeOH in DCM as eluent (20 mL). The volatiles were evaporated under reduced pressure to give a residue. To the residue was added water (20 mL) and DCM (30 mL). The layers were separated, and the aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-10% MeOH in DCM to afford tert-butyl 7-(7-(2,8-dimethylimidazo[1,2-b]pyridazin-6-yl)-5-fluoro-4-oxoquinazolin-3(4H)-yl)-4-azaspiro[2.5]octane-4-carboxylate (700 mg, 41%) as a solid. LCMS (ES, m/z): 519.3 [M+H]⁺. Separation of racemic G110 by supercritical fluid chromatography (SFC) (Condition G6)) (Rₜ=5.180 min, 96 mg) and the second eluting enantiomer (Rₜ=5.808 min, 138 mg). ¹H NMR of G110A (CHCl₃-d, 300 MHz): δ_{H} 8.10 (1H, s), 8.05 (1H, s), 7.87 (1H, d, J=11.9 Hz), 7.82 (1H, s), 7.35 (1H, s), 5.27 (1H, m), 4.27 (1H, d, J=14.0 Hz), 3.09 (1H, t, J=13.0 Hz), 2.76 (3H, s), 2.57 (3H, s), 2.33 (1H, t, J=13.2 Hz), 2.03 (1H, d, J=12.5 Hz), 1.90 (1H, t, J=13.6 Hz), 1.53 (9H, s), 1.38 (2H, m), 0.93-1.01 (1H, m), 0.69 (2H, t, J=8.4 Hz). ¹H NMR of G110B (CHCl₃-d, 300 MHz): δ_{H} 8.10 (1H, s), 8.05 (1H, s), 7.87 (1H, d, J=12.1 Hz), 7.82 (1H, s), 7.35 (1H, s), 5.26 (1H, s), 4.27 (1H, d, J=14.1 Hz), 3.09 (1H, t, J=13.1 Hz), 2.76 (3H, s), 2.57 (3H, s), 2.33 (1H, t, J=13.5 Hz), 2.03 (1H, d, J=12.4 Hz), 1.90 (1H, t, J=14.0 Hz), 1.53 (9H, s), 1.32-1.44 (2H, m), 0.97 (2H, d, J=9.3 Hz), 0.71 (2H, d, J=9.3 Hz).

### Synthesis of Compound 351

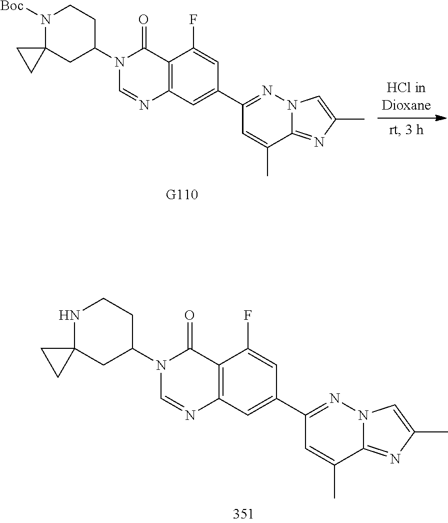 [SMILES: Cc1cn2nc(-c3cc(F)c4c(=O)n(C5CCN(C(=O)OC(C)(C)C)C6(CC6)C5)cnc4c3)cc(C)c2n1 ; Cc1cn2nc(-c3cc(F)c4c(=O)n(C5CCNC6(CC6)C5)cnc4c3)cc(C)c2n1]

To a solution of 7-(7-(2,8-dimethylimidazo[1,2-b]pyridazin-6-yl)-5-fluoro-4-oxoquinazolin-3(4H)-yl)-4-azaspiro[2.5]octane-4-carboxylate (23 mg, 44.4 mol) in methanol (1.0 mL) was added 4.0 M HCl solution in dioxane (2.2 mL, 8.87 mmol). The reaction mixture was stirred at room temperature for 3 h, then concentrated under reduced pressure, diluted with DCM (10 mL), and washed with saturated NaHCO₃ (10 mL). The aqueous phase was extracted with DCM (10 mL×2). The organic layers were combined, dried over Na₂SO₄, filtered, and the filtrate concentrated under reduced pressure to give a residue. The residue was triturated twice with MTBE (8 mL). The resulting solid was collected by filtration, then dried under reduced pressure, dissolved in MeCN/H₂O (1:1), and lyophilized to afford 6-(2,8-dimethylimidazo[1,2-b]pyridazin-6-yl)-2-(4-azaspiro[2.5]octan-7-yl)isoquinolin-1(2H)-one (15 mg, 81%) as a solid. LCMS (ES, m/z): 419.2 [M+H]⁺. ¹H NMR (CDCl3, 300 MHz): δ 8.18 (1H, s), 8.05 (1H, s), 7.86 (1H, d, J=12.2 Hz), 7.82 (1H, s), 7.35 (1H, s), 5.15 (1H, m), 3.26 (1H, d, J=13.4 Hz), 3.01 (1H, t, J=12.6 Hz), 2.76 (3H, s), 2.57 (3H, s), 2.26 (1H, t, J=12.2 Hz), 2.09 (1H, d, J=12.5 Hz), 1.78-1.91 (1H, m), 1.45 (1H, m), 0.73 (2H, d, J=22.1 Hz), 0.59 (2H, m). Deprotection of G110A (Rₜ=5.180 min, 96 mg) afforded Compound 356 (74 mg, 96%). Deprotection of G110B (Rₜ=5.808 min, 138 mg) afforded Compound 357 (108 mg, 97%).

### Example G63: Synthesis of Compound 380

### Synthesis of Intermediate G111

Intermediate G111 was prepared according to the procedure described for the preparation of Compound 384 where in step 1, 6-bromoisoquinolin-1(2H)-one was replaced with 6-bromo-8-fluoroisoquinolin-1(2H)-one. tert-butyl 7-(6-(2,8-dimethylimidazo[1,2-b]pyridazin-6-yl)-8-fluoro-1-oxoisoquinolin-2(1H)-yl)-4-azaspiro[2.5]octane-4-carboxylate was thus obtained as a solid. LCMS (ES, m/z): 518.3 [M+H]⁺. Separation of racemic G111 by supercritical fluid chromatography (SFC) (Condition G6) G111A (Rₜ=3.77 min, 55 mg) and G111B (Rₜ=4.18 min, 47 mg).

### Synthesis of Compound 380

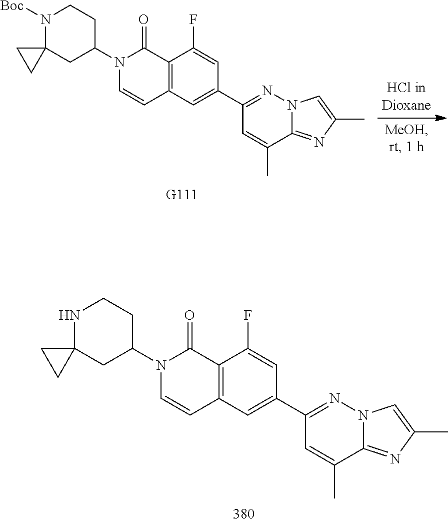 [SMILES: Cc1cn2nc(-c3cc(F)c4c(=O)n(C5CCN(C(=O)OC(C)(C)C)C6(CC6)C5)ccc4c3)cc(C)c2n1 ; Cc1cn2nc(-c3cc(F)c4c(=O)n(C5CCNC6(CC6)C5)ccc4c3)cc(C)c2n1]

To a solution of tert-butyl 7-(6-(2,8-dimethylimidazo[1,2-b]pyridazin-6-yl)-8-fluoro-1-oxoisoquinolin-2(1H)-yl)-4-azaspiro[2.5]octane-4-carboxylate (30 mg, 0.06 mmol) in methanol (0.5 mL) was added 4.0 M HCl solution in dioxane (1 mL). The reaction mixture was stirred at room temperature for 1 h. The volatiles were evaporated under reduced pressure. Ethyl acetate (5 mL) was added and the mixture was sonicated. The suspended material was collected by filtration, washed with EtOAc (5 mL) and dried under vacuum. The solid was dissolved in water (1 mL), basified to pH 10 with 20% NH₄OH added dropwise. This suspension centrifuged, decanted, washed and sonicated with water (1 mL). This cycle was repeated twice and the remaining solid was taken up in acetonitrile/water, then lyophilized to afford the 6-(2,8-dimethylimidazo[1,2-b]pyridazin-6-yl)-8-fluoro-2-(4-azaspiro[2.5]octan-7-yl)isoquinolin-1(2H)-one (20 mg, 83%) as a solid. LCMS (ES, m/z): 418.2 [M+H]⁺. ¹H NMR (CH₂Cl₂-d₂, 400 MHz): δ_{H} 7.86 (1H, s), 7.81 (1H, s), 7.72 (1H, d, J=13.0 Hz), 7.34 (1H, s), 7.28 (1H, d, J=7.6 Hz), 6.61 (1H, d, J=7.6 Hz), 5.26 (1H, m), 3.17 (1H, m), 2.98 (1H, m), 2.69 (3H, s), 2.50 (3H, s), 2.16 (1H, m), 1.96 (1H, s), 1.73 (1H, m), 1.46 (1H, m), 1.33 (1H, d, J=12.4 Hz), 0.73 (1H, m), 0.66 (1H, m), 0.56 (2H, m). Deprotection of the first eluting enantiomer of 1 (Rₜ=3.77 min, 55 mg) using the above-mentioned conditions afforded Compound 425 (51 mg, 99%). Deprotection of the second eluting enantiomer of 1 (Rₜ=4.18 min, 47 mg) using the above-mentioned conditions afforded Compound 426 (44 mg, 99%).

### Example G64: Synthesis of Compound

### Synthesis of Intermediate G112

Intermediate G112 was prepared according to the procedure described for the preparation of Compound 384 steps 1 and 2, where in step 1, 6-bromoisoquinolin-1(2H)-one was replaced with 6-bromo-1-oxo-1,2-dihydroisoquinoline-8-carbonitrile. LCMS (ES, m/z): 525.3 [M+H]⁺.

### Synthesis of Compound 387

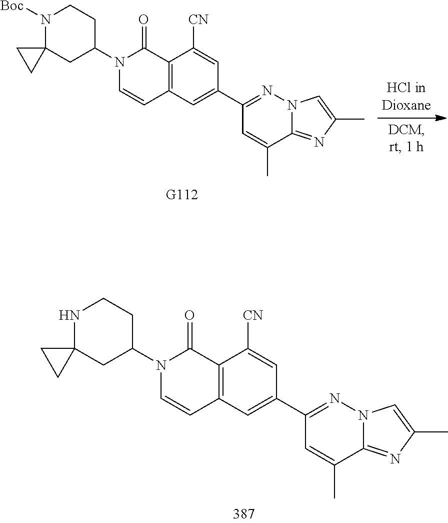 [SMILES: Cc1cn2nc(-c3cc(C#N)c4c(=O)n(C5CCN(C(=O)OC(C)(C)C)C6(CC6)C5)ccc4c3)cc(C)c2n1 ; Cc1cn2nc(-c3cc(C#N)c4c(=O)n(C5CCNC6(CC6)C5)ccc4c3)cc(C)c2n1]

To a solution of tert-butyl 7-(8-cyano-6-(2,8-dimethylimidazo[1,2-b]pyridazin-6-yl)-1-oxoisoquinolin-2(1H)-yl)-4-azaspiro[2.5]octane-4-carboxylate (13 mg, 0.025 mmol) in DCM (1.00 mL) was added 4 M HCl in dioxane (1.00 mL). The reaction mixture was stirred at room temperature for 1 h. The volatiles were evaporated under reduced pressure. Ethyl acetate (3 mL) was added and the mixture was sonicated. The suspended material was collected by filtration, washed with EtOAc (2 mL) and dried under vacuum to afford Compound 387 (10.6 mg, 86%) as a solid. LCMS (ES, m/z): 213.2 [M+2H]⁺. ¹H NMR (DMSO-d₆, 400 MHz): δ_{H} 9.60-9.67 (0.3H, m)*, 8.90-8.97 (0.3H, m)*, 8.63 (1H, d, J=1.7 Hz), 8.32 (1H, t, J=3.4 Hz), 8.12-8.22 (1H, m), 7.62 (1H, d, J=7.5 Hz), 6.97 (1H, d, J=7.5 Hz), 5.02-5.10 (1H, m), 3.90-4.10 (0.5H, m)*, 3.47-3.51 (1H, m), 3.12-3.22 (1H, m), 2.71-2.78 (1H, m), 2.65-2.68 (4H, m), 2.27-2.34 (1H, m), 2.07-2.12 (1H, m), 1.48-1.52 (1H, m), 1.09-1.10 (2H, m), 0.83 (2H, s).

### Example G65: Synthesis of Compound 354

### Synthesis of Intermediate G113

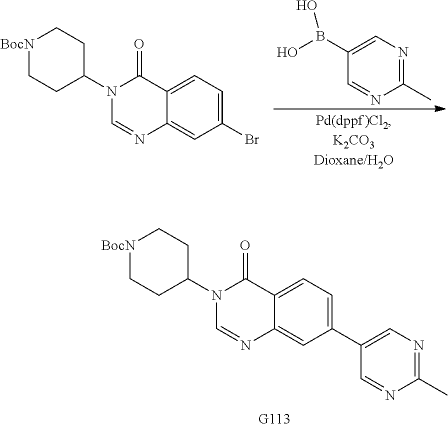

To a solution of tert-butyl 4-(7-bromo-4-oxoquinazolin-3(4H)-yl)piperidine-1-carboxylate (100 mg, 246 umol) in dioxane (4.0 mL) were added potassium carbonate (102 mg, 737 umol), (2-Methylpyrimidin-5-yl)boronic acid (40.6 mg, 295 umol) and water (1.0 mL). The reaction mixture was degassed for 2 min with nitrogen, then Pd(dppf)Cl₂·DCM (20.5 mg, 24.6 umol) was added. The resulting mixture was heated at 100° C. for 1 h, then purified by reverse phase chromatography to afford tert-butyl 4-(7-(2-methylpyrimidin-5-yl)-4-oxoquinazolin-3(4H)-yl)piperidine-1-carboxylate (86.0 mg, 83%) as a solid. LCMS (ES, m/z): 422.2 [M+H]⁺.

### Synthesis of Compound 354

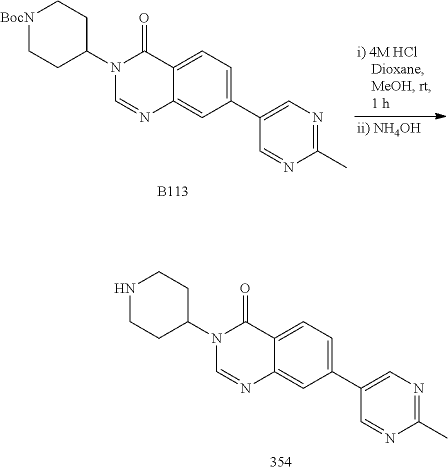 [SMILES: "B113" ; Cc1ncc(-c2ccc3c(=O)n(C4CCN(C(=O)OC(C)(C)C)CC4)cnc3c2)cn1 ; Cc1ncc(-c2ccc3c(=O)n(C4CCNCC4)cnc3c2)cn1]

To a solution of tert-butyl 4-(7-(2-methylpyrimidin-5-yl)-4-oxoquinazolin-3(4H)-yl)piperidine-1-carboxylate (76 mg, 0.18 mmol) in methanol (1.0 mL) was added 4 M HCl in dioxane (1.0 mL). The reaction mixture was stirred at room temperature for 1 h, then concentrated under reduced pressure, diluted with ethyl acetate (3 mL), and sonicated to form a suspension. The suspension was filtered to collect the solid. The solid was washed with ethyl acetate (5 mL) and dried under vacuum. The resulting solid was dissolved in water (1 mL), basified to pH 10 with 20% NH₄OH, then extracted with DCM. The organic layer was concentrated under reduced pressure to afford 7-(2-methylpyrimidin-5-yl)-3-(piperidin-4-yl)quinazolin-4(3H)-one (41 mg, 71%). LCMS (ES, m/z): 322.2 [M+H]⁺. ¹H NMR (CHCl₃-d, 400 MHz): δ_{H} 8.96 (2H, s), 8.44 (1H, d, J=8.3 Hz), 8.21 (1H, s), 7.90 (1H, d, J=1.7 Hz), 7.70 (1H, dd, J=8.3, 1.8 Hz), 4.93-4.99 (1H, m), 3.29 (2H, d, J=12.3 Hz), 2.83-2.89 (5H, m), 2.01 (2H, d, J=11.8 Hz), 1.84-1.93 (2H, m).

### Example G66: Synthesis of Compound 355

### Synthesis of Intermediate G114

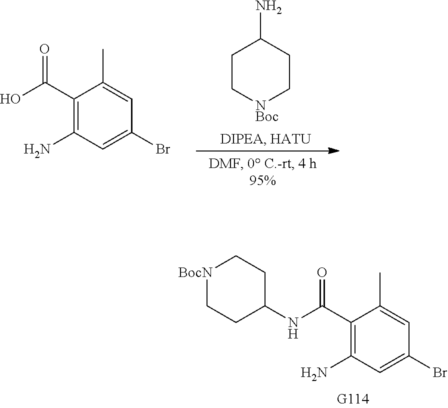 [SMILES: Cc1cc(Br)cc(N)c1C(=O)O ; CC(C)(C)OC(=O)N1CCC(N)CC1 ; Cc1cc(Br)cc(N)c1C(=O)NC1CCN(C(=O)OC(C)(C)C)CC1]

To a mixture of 2-amino-4-bromo-6-methylbenzoic acid (200 mg, 0.87 mmol) and tert-butyl 4-aminopiperidine-1-carboxylate (202 mg, 1.01 mmol) in DMF (4 mL) was added DIPEA (0.46 mL, 2.61 mmol), followed by HATU (363 mg, 0.956 mmol) at 0° C. The reaction mixture was stirred at 0° C. for 1 h, then at room temperature for an additional 3 h. The resulting mixture was diluted with ethyl acetate (40 mL) and washed with a saturated aqueous solution of NH₄Cl (25 mL), followed by a saturated aqueous solution of NaHCO₃ (25 mL), and brine (25 mL). The organic layer was dried over Na₂SO₄, filtered, and the filtrate concentrated under reduced pressure to give a residue. The residue was purified by column chromatography on silica gel (12 g) using a gradient of 0-100% ethyl acetate in hexane to afford tert-butyl 4-(2-amino-4-bromo-6-methylbenzamido)piperidine-1-carboxylate (340 mg, 95%) as a solid. LCMS (ES, m/z): 312.1 [M-Boc+H]⁺. ¹H NMR (CHCl₃-d, 400 MHz): δ_{H} 6.71 (2H, d, J=6.6 Hz), 5.66 (1H, d, J=8.0 Hz), 4.20 (2H, s), 4.14-4.06 (2H, m), 2.91 (2H, t, J=12.6 Hz), 2.27 (3H, s), 2.02 (2H, d, J=15.1 Hz), 1.46 (9H, s), 1.41-1.35 (2H, m).

### Synthesis of Intermediate G11S

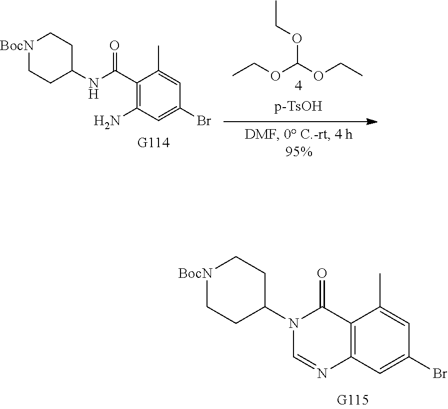

To a mixture of tert-butyl 4-(2-amino-4-bromo-6-methylbenzamido)piperidine-1-carboxylate (330 mg, 0.80 mmol) and triethyl orthoformate (1.36 mL, 8.0 mmol) in THF (10 mL) was added p-TsOH (76 mg, 0.40 mmol). The reaction mixture was heated at 100° C. for 24 h, then cooled to room temperature. The resulting mixture was concentrated under reduced pressure to give a residue. The residue was dissolved in ethyl acetate (50 mL), then washed with a saturated solution of NaHCO₃ (40 mL) and brine (2×30 mL). The organic layer was dried over Na₂SO₄, filtered, and the filtrate concentrated under reduced pressure to give a residue. The residue was purified by column chromatography on silica gel (12 g) using a gradient of 0-100% ethyl acetate in hexane to afford tert-butyl 4-(7-bromo-5-methyl-4-oxoquinazolin-3(4H)-yl)piperidine-1-carboxylate (250 mg, 74%) as a solid. LCMS (ES, m/z): 422.1 [M+H]⁺. ¹H NMR (CHCl₃-d, 400 MHz): δ_{H} 8.01 (1H, s), 7.71 (1H, s), 7.40 (1H, s), 4.92-4.85 (1H, m), 4.35 (2H, br s), 3.43-3.38 (1H, m), 2.91-2.90 (1H, m), 2.85 (3H, s), 1.94 (2H, d, J=12.1 Hz), 1.82-1.73 (1H, m), 1.62-1.58 (1H, m), 1.50 (9H, s).

### Synthesis of Intermediate G116

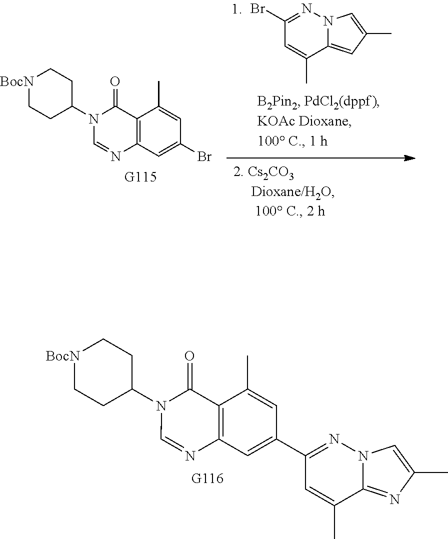 [SMILES: Cc1cc(Br)cc2ncn(C3CCN(C(=O)OC(C)(C)C)CC3)c(=O)c12 ; Cc1cc2c(C)cc(Br)nn2c1 ; Cc1cn2nc(-c3cc(C)c4c(=O)n(C5CCN(C(=O)OC(C)(C)C)CC5)cnc4c3)cc(C)c2n1]

A mixture of 6-bromo-2,8-dimethylimidazo[1,2-b]pyridazine 6 (167 mg, 0.74 mmol), B₂Pin₂ (188 mg, 0.74 mmol), Pd(dppf)Cl₂·DCM (41.6 mg, 0.0568 mmol), and KOAc (167 mg, 1.70 mmol) in dioxane (4.0 mL) was heated to 100° C. for 1 h. To the reaction mixture was added a solution of tert-butyl 4-(7-bromo-5-methyl-4-oxoquinazolin-3(4H)-yl)piperidine-1-carboxylate, (240 mg, 0.568 mmol) in dioxane (3.0 mL), followed by Cs₂CO₃ (555 mg, 1.70 mmol) and H₂O (0.8 mL) under argon atmosphere. The reaction mixture was heated at 90° C. for 2 h, then cooled to room temperature. The reaction mixture was filtered over celite using 20% methanol in DCM (10 mL) as eluent. The filtrate was concentrated under reduced pressure to give a residue. Water (20 mL) and DCM (30 mL) were added to the residue, the layers were separated and the aqueous layer was extracted with DCM (3×20 mL). The organic layers were combined, dried over Na₂SO₄, filtered, and the filtrate concentrated under reduced pressure to give a residue. The residue was purified by column chromatography on silica gel (24 g) using a gradient of 0-100% ethyl acetate in hexane to afford tert-butyl 4-(7-(2,8-dimethylimidazo[1,2-b]pyridazin-6-yl)-5-methyl-4-oxoquinazolin-3(4H)-yl)piperidine-1-carboxylate (118 mg, 42%) as a solid. LCMS (ES, m/z): 489.3 [M+H]⁺.

### Synthesis of Compound 355

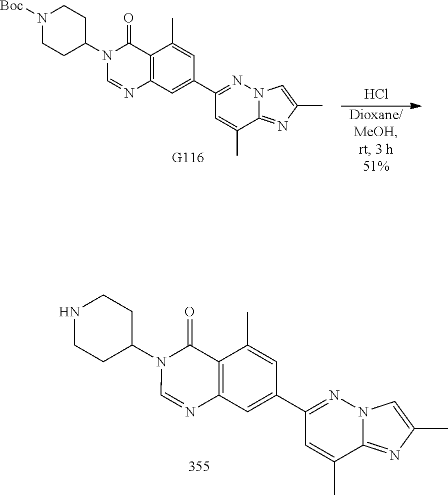 [SMILES: Cc1cn2nc(-c3cc(C)c4c(=O)n(C5CCN(C(=O)OC(C)(C)C)CC5)cnc4c3)cc(C)c2n1 ; Cl ; Cc1cn2nc(-c3cc(C)c4c(=O)n(C5CCNCC5)cnc4c3)cc(C)c2n1]

To a solution of tert-butyl 4-(7-(2,8-dimethylimidazo[1,2-b]pyridazin-6-yl)-5-methyl-4-oxoquinazolin-3(4H)-yl)piperidine-1-carboxylate 7 (118 mg, 0.242 mmol) in MeOH (5.0 mL) was added 4.0 M HCl solution in dioxane (0.90 mL, 3.62 mmol). The reaction mixture was stirred at rt for 3 h. The volatiles were evaporated under reduced pressure. The residue was diluted with DCM (10 mL) and washed with a saturated solution of NaHCO₃ (15 mL). The aqueous phase was extracted with DCM (10 mL×2). The organic layers were combined, dried over Na₂SO₄, filtered, and concentrated under reduced pressure. The crude product was purified by column chromatography on silica gel (12 g) using a gradient of 0-20% MeOH:NH₄OH (9:1) in DCM to afford 7-(2,8-dimethylimidazo[1,2-b]pyridazin-6-yl)-5-methyl-3-(4-azaspiro[2.5]octan-7-yl)quinazolin-4(3H)-one (48 mg, 51%) as a solid. LCMS (ES, m/z): 389.2 [M+H]⁺. ¹H NMR (CHCl₃-d, 400 MHz): δ_{H} 8.14 (1H, s), 8.02 (1H, s), 7.88 (1H, s), 7.79 (1H, s), 7.32 (1H, s), 4.97-4.89 (1H, m), 3.29 (2H, d, J=12.2 Hz), 2.97 (3H, s), 2.85 (2H, t, J=12.0 Hz), 2.72 (3H, s), 2.53 (3H, s), 2.00 (2H, d, J=11.8 Hz), 1.86 (2H, qd, J=12.1, 3.9 Hz).

### Example G67: Synthesis of Compound 409

### Synthesis of Intermediate G117

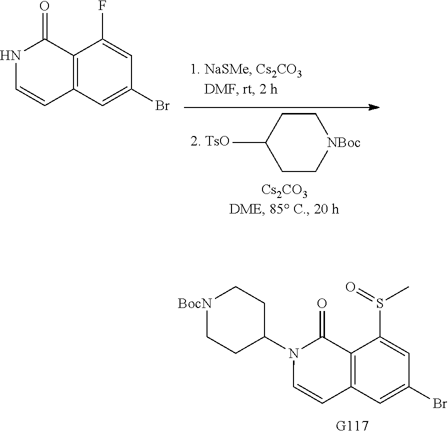 [SMILES: O=c1[nH]ccc2cc(Br)cc(F)c12 ; CC1CCN(C(=O)OC(C)(C)C)CC1 ; CS(=O)c1cc(Br)cc2ccn(C3CCN(C(=O)OC(C)(C)C)CC3)c(=O)c12]

A mixture of 6-bromo-8-fluoro-1,2-dihydroisoquinolin-1-one (25 mg, 0.10 mmol), sodium thiomethoxide (25 mg, 0.36 mmol), and Cs₂CO₃ (33 mg, 0.10 mmol) in DMF (0.5 mL) was stirred at room temperature for 2 h. To the reaction mixture was added a solution of 1-boc-4-(tosyloxy)piperidine (115 mg, 0.31 mmol) in a mixture of DMF (0.5 mL) and DME (1.5 mL), followed by Cs₂CO₃ (101 mg, 0.31 mmol). The reaction mixture was heated to 85° C. for 20 h, then cooled to room temperature. The reaction mixture was filtered, and the filtrate was concentrated under reduced pressure to give a residue. The residue was purified by column chromatography on silica gel using a gradient of 0-100% ethyl acetate in hexane to afford tert-butyl 4-(6-bromo-8-(methylsulfinyl)-1-oxoisoquinolin-2(1H)-yl)piperidine-1-carboxylate (21 mg, 43%) as a solid. LCMS (ES, m/z): 469.1 [M+H]⁺.

### Synthesis of Intermediate G118

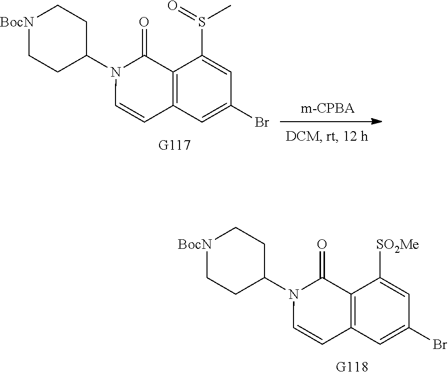 [SMILES: CS(=O)c1cc(Br)cc2ccn(C3CCN(C(=O)OC(C)(C)C)CC3)c(=O)c12 ; CC(C)(C)OC(=O)N1CCC(n2ccc3cc(Br)cc(S(C)(=O)=O)c3c2=O)CC1]

To a solution of tert-butyl 4-(6-bromo-8-(methylsulfinyl)-1-oxoisoquinolin-2(1H)-yl)piperidine-1-carboxylate (21 mg, 0.044 mmol) in DCM (2.0 mL) was added m-CPBA (18 mg, 0.089 mmol) at room temperature. The reaction mixture was stirred at room temperature for 12 h, then concentrated under reduced pressure to give a residue. Water (5 mL) and DCM (5 mL) were added to the residue, then the layers were separated. The aqueous layer was extracted with DCM (3×5 mL). The organic layers were combined, dried over Na₂SO₄, filtered, and the filtrate concentrated under reduced pressure to give a residue. The residue was purified by column chromatography on silica gel using a gradient of 0-5% methanol in ethyl acetate to afford tert-butyl 4-(6-bromo-8-(methylsulfonyl)-1-oxoisoquinolin-2(1H)-yl)piperidine-1-carboxylate (20 mg, 92%) as a solid. LCMS (ES, m/z): 507.1 [M+Na]⁺.

### Synthesis of Intermediate G119

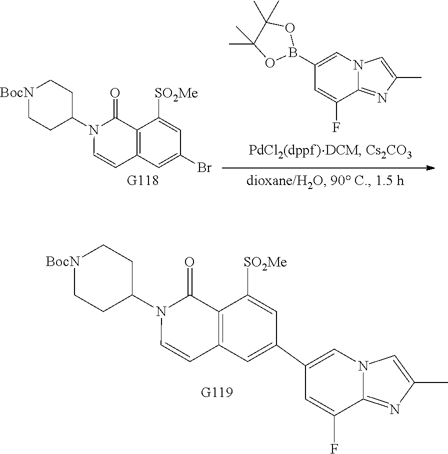

A mixture of tert-butyl 4-(6-bromo-8-(methylsulfonyl)-1-oxoisoquinolin-2(1H)-yl)piperidine-1-carboxylate (20 mg, 0.041 mmol), 8-fluoro-2-methyl-6-(4,4,5,5-tetramethyl-1,3,2-dioxaborolan-2-yl)imidazo[1,2-a]pyridine (17 mg, 0.062 mmol), Cs₂CO₃ (34 mg, 0.10 mmol), and Pd(dppf)Cl₂·DCM (3.5 mg, 0.0041 mmol) in dioxane (0.7 mL) and water (0.08 mL) was heated to 90° C. for 1.5 h, then cooled to room temperature. The reaction mixture was filtered over celite using 10% methanol in DCM as eluent. The filtrate was concentrated under reduced pressure to give a residue. The residue was purified by column chromatography on silica gel using a gradient of 0-5% methanol in ethyl acetate to afford tert-butyl 4-(6-(8-fluoro-2-methylimidazo[1,2-a]pyridin-6-yl)-8-(methylsulfonyl)-1-oxoisoquinolin-2(1H)-yl)piperidine-1-carboxylate (20 mg, 88%) as a solid. LCMS (ES, m/z): 555.2 [M+H]⁺.

### Synthesis of Compound 409

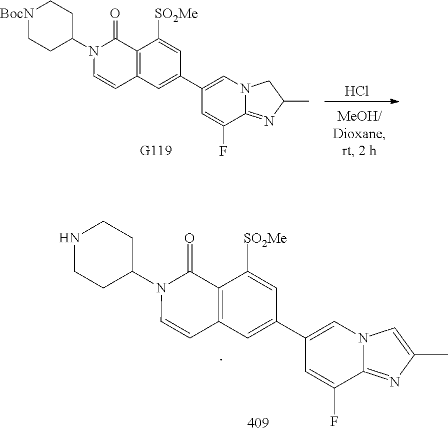 [SMILES: CC1CN2C=C(c3cc(S(C)(=O)=O)c4c(=O)n(C5CCN(C(=O)OC(C)(C)C)CC5)ccc4c3)C=C(F)C2=N1 ; Cl ; Cc1cn2cc(-c3cc(S(C)(=O)=O)c4c(=O)n(C5CCNCC5)ccc4c3)cc(F)c2n1]

To a solution of tert-butyl 4-(6-(8-fluoro-2-methylimidazo[1,2-a]pyridin-6-yl)-8-(methylsulfonyl)-1-oxoisoquinolin-2(1H)-yl)piperidine-1-carboxylate (20 mg, 0.036 mmol) in methanol (1.00 mL) was added 4 M HCl solution in dioxane (1.0 mL, 4.0 mmol). The reaction mixture was stirred at room temperature for 2 h, then concentrated under reduced pressure to give a residue. The residue was purified by reverse phase chromatography (Condition G3, Gradient 1) to afford 6-(8-fluoro-2-methylimidazo[1,2-a]pyridin-6-yl)-8-(methylsulfonyl)-2-(piperidin-4-yl)isoquinolin-1(2H)-one (11 mg, 62%) as solid. LCMS (ES, m/z): 455.1 [M+H]⁺. ¹H NMR (DMSO-d₆, 400 MHz): δ_{H} 9.29 (1H, s), 9.22 (1H, d, J=11.9 Hz), 9.10 (1H, s), 8.58 (1H, d, J=1.9 Hz), 8.51 (1H, s), 8.24 (1H, d, J=11.9 Hz), 8.17 (1H, s), 7.63 (1H, d, J=7.4 Hz), 6.94 (1H, d, J=7.4 Hz), 5.11 (1H, m), 3.70 (3H, s), 3.45 (2H, d, J=15.1 Hz), 3.16 (2H, m), 2.23-2.30 (2H, m), 1.99 (2H, d, J=12.7 Hz).

### Example G68: Synthesis of Compound 423

### Synthesis of Intermediate G120

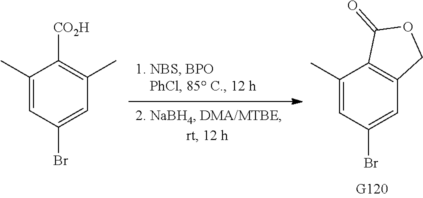

To a mixture of 4-bromo-2,6-dimethylbenzoic acid (2.00 g, 8.73 mmol) and NBS (3.42 g, 19.2 mmol) in PhCl (36.0 mL) was added benzoyl peroxide (0.211 g, 0.873 mmol). The reaction mixture was heated to 85° C. for 12 h. To the resulting mixture was added an aqueous solution of sodium bisulfite (40% w/w, 20 mL), and the layers were separated. The organic phase was washed with a saturated aqueous solution of NaHCO₃ (20 mL), then concentrated under reduced pressure to give a residue. The residue was dissolved in DMA (9.0 mL). To the resulting solution was added a mixture of NaBH₄ (0.661 g, 17.5 mmol) in MTBE (14 mL) and DMA (9.0 mL). The resulting mixture was stirred at room temperature for 12 h, then diluted with a solution of HCl (36% w/w, 5.0 mL) in water (7.0 mL). The aqueous phase was extracted with diethyl ether (3×20 mL). The organic layers were combined, dried over Na₂SO₄, filtered, and the filtrate concentrated under reduced pressure to give a residue. The residue was purified by column chromatography on silica gel (80 g) using a gradient of 0-100% ethyl acetate in hexane to afford 5-bromo-7-methylisobenzofuran-1(3H)-one (1.25 g, 63%) as a solid. LCMS (ES, m/z): 227.0 [M+H]⁺. ¹H NMR (CHCl₃-d, 400 MHz): δ_{H} 7.44 (2H, s), 5.22 (2H, s), 2.66 (3H, s).

### Synthesis of Intermediate G121

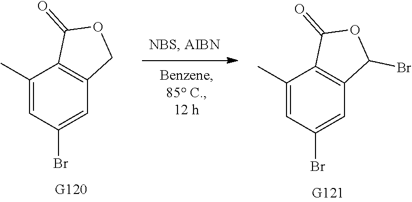

To a solution of 5-bromo-7-methylisobenzofuran-1(3H)-one (0.870 g, 3.83 mmol) in benzene (40 mL) was added NBS (0.758 g, 4.21 mmol), followed by AIBN (31.5 mg, 0.192 mmol) at room temperature. The reaction mixture was heated at 85° C. for 12 h, then cooled to room temperature and concentrated under reduced pressure to give a residue. The residue was purified by column chromatography on silica gel (80 g) using a gradient of 0-100% ethyl acetate in hexane to afford 3,5-dibromo-7-methylisobenzofuran-1(3H)-one (1.10 g, 94%) as a solid. ¹H NMR (CHCl₃-d, 400 MHz): δ_{H} 7.58 (1H, s), 7.52 (1H, s), 7.30 (1H, s), 2.66 (3H, s).

### Synthesis of Intermediate G122

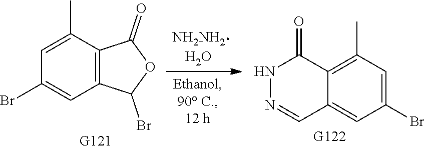

To a solution of 3,5-dibromo-7-methylisobenzofuran-1(3H)-one 3 (900 mg, 2.94 mmol) in EtOH (35 mL) was added hydrazine monohydrate (65% in water, 0.66 mL, 8.82 mmol) and stirred at rt for 10 min. The mixture was then heated at 90° C. for 12 h. Upon completion, the reaction mixture was concentrated under reduced pressure to afford 6-bromo-8-methylphthalazin-1(2H)-one (530 mg, 75%) as a solid. LCMS (ES, m/z): 239.0 [M+H]⁺. ¹H NMR (DMSO-d6, 400 MHz): δ_{H} 12.52 (1H, s), 8.20 (1H, s), 7.99 (1H, s), 7.78 (1H, s), 2.77 (3H, s).

### Synthesis of Intermediate G123

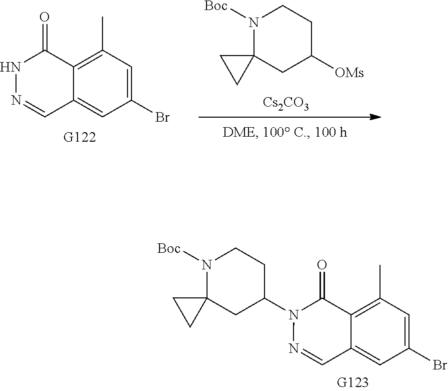

To a mixture of 6-bromo-8-methylphthalazin-(2H)-one-2 (410 mg, 1.71 mmol) in DME (85 mL) was added Cs₂CO₃ (1.68 g, 5.14 mmol). The resulting mixture was stirred for 20 min, then tert-butyl 8-((methylsulfonyl)oxy)-5-azaspiro[2.5]octane-5-carboxylate (1.10 g, 3.60 mmol) was added. The reaction mixture was stirred at 100° C. for 100 h, then cooled to room temperature. The resulting mixture was filtered through a celite, and the filtrate was concentrated under reduced pressure to give a residue. The residue was taken up in DCM (50 mL) and washed with a saturated solution of NaHCO₃ (30 mL). The organic layer was dried over Na₂SO₄, filtered, and the filtrate concentrated under reduced pressure to give a residue. The residue was purified by column chromatography on silica gel (40 g) using a gradient of 0-100% ethyl acetate in hexane to afford tert-butyl 7-(6-bromo-8-methyl-1-oxophthalazin-2(1H)-yl)-4-azaspiro[2.5]octane-4-carboxylate (0.270 g, 35%) as a liquid. LCMS (ES, m/z): 470.1 [M+Na]⁺. ¹H NMR (CHCl₃-d, 400 MHz): δ_{H} 8.01 (1H, s), 7.62 (2H, d, J=9.1 Hz), 5.39-5.31 (1H, m), 4.16 (1H, t, J=13.3 Hz), 3.04 (1H, t, J=12.9 Hz), 2.91 (3H, s), 2.50-2.43 (1H, m), 2.06-1.97 (1H, m), 1.82 (1H, d, J=12.8 Hz), 1.50 (9H, s), 1.14 (1H, dd, J=12.5, 4.2 Hz), 0.89-0.87 (1H, m), 0.66-0.59 (2H, m).

### Synthesis of Intermediate G124

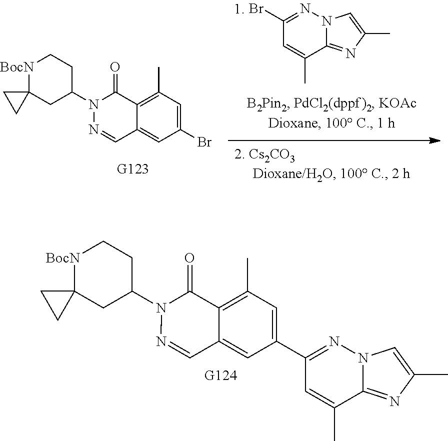 [SMILES: Cc1cc(Br)cc2cnn(C3CCN(C(=O)OC(C)(C)C)C4(CC4)C3)c(=O)c12 ; Cc1cn2nc(Br)cc(C)c2n1 ; Cc1cn2nc(-c3cc(C)c4c(=O)n(C5CCN(C(=O)OC(C)(C)C)C6(CC6)C5)ncc4c3)cc(C)c2n1]

A mixture of 6-bromo-2,8-dimethylimidazo[1,2-b]pyridazine (241 mg, 1.06 mmol), B₂Pin₂ (270 mg, 1.06 mmol), Pd(dppf)Cl₂·DCM (65 mg, 0.0887 mmol), and KOAc (191 mg, 1.95 mmol) in dioxane (16 mL) was heated to 100° C. for 1 h. To the reaction mixture was added a solution of tert-butyl 4-(7-bromo-5-methyl-4-oxoquinazolin-3(4H)-yl)piperidine-1-carboxylate (265 mg, 0.591 mmol) in dioxane (5.0 mL), followed by Cs₂CO₃ (578 mg, 1.77 mmol) and water (4.0 mL) under argon atmosphere. The reaction mixture was heated at 90° C. for 2 h, then cooled to room temperature. The reaction mixture was filtered over celite using 20% methanol in DCM (10 mL) as eluent. The filtrate was concentrated under reduced pressure to give a residue. Water (20 mL) and DCM (30 mL) were added to the residue, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel (40 g) using a gradient of 0-100% ethyl acetate in hexane to afford tert-butyl 7-(6-(2,8-dimethylimidazo[1,2-b]pyridazin-6-yl)-8-methyl-1-oxophthalazin-2(1H)-yl)-4-azaspiro[2.5]octane-4-carboxylate (0.230 g, 76%) as a liquid. LCMS (ES, m/z): 515.3 [M+H]⁺. ¹H NMR (CHCl₃-d, 400 MHz): δ_{H} 8.12 (1H, s), 7.98 (2H, d, J=14.0 Hz), 7.74 (1H, s), 7.41 (1H, br s), 5.32-5.29 (1H, m), 4.12 (1H, d, J=12.9 Hz), 2.97 (4H, s), 2.68 (3H, s), 2.48-2.44 (4H, m), 1.97 (1H, s), 1.79 (1H, d, J=12.6 Hz), 1.44 (9H, s), 1.26 (1H, br s), 1.10 (1H, d, J=13.2 Hz), 0.82 (1H, s), 0.57 (2H, d, J=10.2 Hz).

### Synthesis of Compound 423

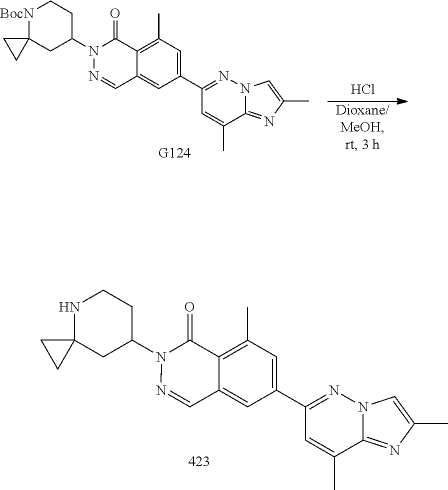 [SMILES: Cc1cn2nc(-c3cc(C)c4c(=O)n(C5CCN(C(=O)OC(C)(C)C)C6(CC6)C5)ncc4c3)cc(C)c2n1 ; Cl ; Cc1cn2nc(-c3cc(C)c4c(=O)n(C5CCNC6(CC6)C5)ncc4c3)cc(C)c2n1]

To a solution of tert-butyl 7-(6-(2,8-dimethylimidazo[1,2-b]pyridazin-6-yl)-8-methyl-1-oxophthalazin-2(1H)-yl)-4-azaspiro[2.5]octane-4-carboxylate (15.0 mg, 0.0292 mmol) in methanol (0.6 mL) was added 4.0 M HCl solution in dioxane (0.13 mL, 0.526 mmol). The reaction mixture was stirred at room temperature for 3 h, then concentrated under reduced pressure and diluted with DCM (5.0 mL). The resulting mixture was washed with a saturated solution of NaHCO₃ (5.0 mL). The aqueous phase was extracted with DCM (2×5.0 mL). The organic layers were combined, dried over Na₂SO₄, filtered, and the filtrate concentrated under reduced pressure to give a residue. The residue was purified by column chromatography on silica gel (4 g) using a gradient of 0-20% methanol:NH₄OH (9:1) in DCM to afford 6-(2,8-dimethylimidazo[1,2-b]pyridazin-6-yl)-8-methyl-2-(4-azaspiro[2.5]octan-7-yl)phthalazin-1(2H)-one (3.1 mg, 26%) as a solid. LCMS (ES, m/z): 415.2 [M+H]⁺. ¹H NMR (CHCl₃-d, 400 MHz): δ_{H} 8.20 (1H, s), 8.05 (2H, d, J=10.5 Hz), 7.80 (1H, s), 7.33 (1H, s), 5.36-5.30 (1H, m), 3.24 (1H, d, J=13.3 Hz), 3.04-2.99 (4H, m), 2.74 (3H, s), 2.55 (3H, s), 2.46 (1H, t, J=12.2 Hz), 2.03-1.97 (1H, m), 1.33-1.25 (2H, m), 0.77 (2H, d, J=26.4 Hz), 0.59-0.52 (2H, m).

### Example G69: Synthesis of Compound 466

### Synthesis of Intermediate G125

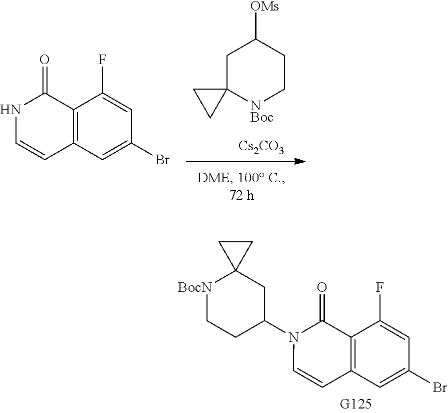

To a mixture of 6-bromo-8-fluoroisoquinolin-1(2H)-one (0.856 g, 3.54 mmol) in DME (45 mL) was added Cs₂CO₃ (1.92 g, 5.89 mmol). The reaction mixture was stirred for 20 min. To the resulting suspension was added tert-butyl 8-((methylsulfonyl)oxy)-5-azaspiro[2.5]octane-5-carboxylate (0.60 g, 1.96 mmol). The reaction mixture was stirred at 100° C. for 72 h, then cooled to room temperature and filtered over celite. The filtrate was concentrated under reduced pressure to give a residue. The residue was taken up in DCM (30 mL) and washed with a saturated solution of NaHCO₃ (20 mL). The organic layer was dried over Na₂SO₄, filtered, and the filtrate concentrated under reduced pressure to give a residue. The residue was purified by column chromatography on silica gel (80 g) using a gradient of 0-100% ethyl acetate in hexane to afford tert-butyl 7-(6-bromo-8-fluoro-1-oxoisoquinolin-2(1H)-yl)-4-azaspiro[2.5]octane-4-carboxylate (0.236 g, 27%) as a liquid. LCMS (ES, m/z): 473.1 [M+Na]⁺. ¹H NMR (CHCl₃-d, 400 MHz): δ_{H} 7.41 (1H, s), 7.23 (1H, dd, J=10.8, 1.8 Hz), 7.10 (1H, d, J=7.6 Hz), 6.38 (1H, d, J=7.6 Hz), 5.39-5.31 (1H, m), 4.18 (1H, d, J=13.3 Hz), 3.04 (1H, t, J=12.8 Hz), 2.14 (1H, br s), 1.88 (1H, d, J=12.3 Hz), 1.74-1.63 (1H, m), 1.48 (9H, s), 1.35-1.29 (1H, n), 1.20 (1H, dd, J=12.7, 4.1 Hz), 0.89 (1H, q, J=7.4 Hz), 0.68-0.60 (2H, m)

### Synthesis of Intermediate G126

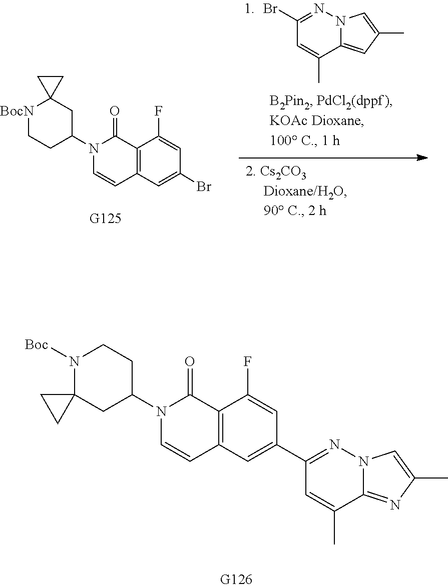 [SMILES: CC(C)(C)OC(=O)N1CCC(n2ccc3cc(Br)cc(F)c3c2=O)CC12CC2 ; Cc1cc2c(C)cc(Br)nn2c1 ; Cc1cn2nc(-c3cc(F)c4c(=O)n(C5CCN(C(=O)OC(C)(C)C)C6(CC6)C5)ccc4c3)cc(C)c2n1]

A mixture of 6-bromo-2,8-dimethylimidazo[1,2-b]pyridazine (0.21 g, 0.935 mmol), B₂Pin₂ (0.237 g, 0.935 mmol), Pd(dppf)Cl₂·DCM (57 mg, 0.078 mmol), and KOAc (0.168 g, 1.71 mmol) in dioxane (13 mL) was heated to 100° C. for 1 h. To the reaction mixture was added a solution of tert-butyl 7-(6-bromo-8-fluoro-1-oxophthalazin-2(1H)-yl)-4-azaspiro[2.5]octane-4-carboxylate (0.234 g, 0.52 mmol) in dioxane (13 mL), followed by Cs₂CO₃ (0.51 g, 1.56 mmol) and water (3 mL) under argon atmosphere. The reaction mixture was heated at 90° C. for 2 h, then cooled to room temperature and filtered over celite using 20% methanol in DCM as eluent (10 mL). The filtrate was concentrated under reduced pressure to give a residue. Water (10 mL) and DCM (20 mL) were added to the residue, and the layers were separated. The aqueous layer was extracted with DCM (3×10 mL). The organic layers were combined, dried over Na₂SO₄, filtered, and the filtrate concentrated under reduced pressure to give a residue. The residue was purified by column chromatography on silica gel (40 g) using a gradient of 0-100% ethyl acetate in hexane to afford tert-butyl 7-(6-(2,8-dimethylimidazo[1,2-b]pyridazin-6-yl)-8-fluoro-1-oxoisoquinolin-2(1H)-yl)-4-azaspiro[2.5]octane-4-carboxylate (0.171 g, 64%) as a solid. LCMS (ES, m/z): 518.3 [M+H]⁺. ¹H NMR (CHCl₃-d, 400 MHz): δ_{H} 7.81 (2H, d, J=13.2 Hz), 7.73 (1H, d, J=12.2 Hz), 7.30 (1H, s), 7.15 (1H, d, J=7.6 Hz), 6.59 (1H, d, J=7.6 Hz), 5.45-5.38 (1H, m), 4.23-4.19 (1H, m), 3.08 (1H, t, J=12.7 Hz), 2.73 (3H, s), 2.55 (3H, s), 2.23-2.13 (1H, m), 1.93 (1H, d, J=12.2 Hz), 1.79-1.70 (1H, m), 1.51 (9H, s), 1.38-1.32 (1H, m), 1.25 (1H, dd, J=11.8, 4.1 Hz), 0.93-0.88 (1H, m), 0.72-0.63 (2H, m).

### Synthesis of Intermediate G127

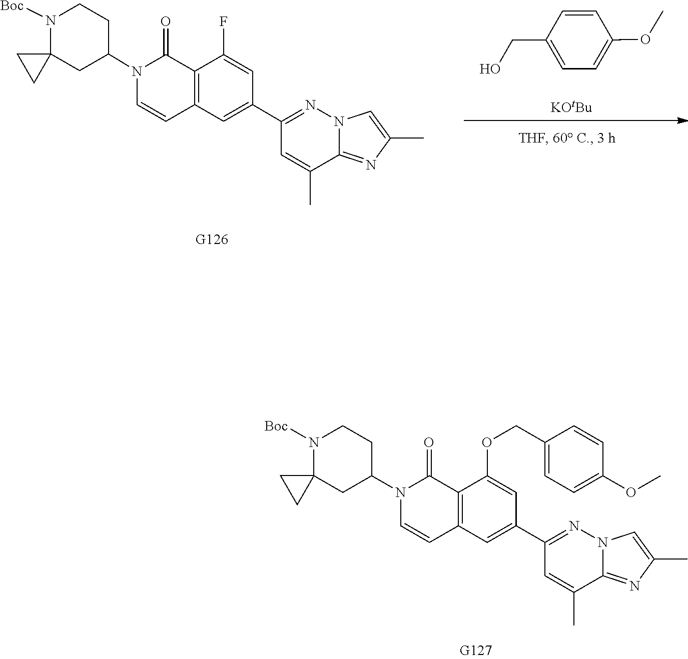

To a solution of 4-methoxybenzyl alcohol (23 mg, 0.16 mmol) in THF (6.0 mL) was added a solution of KO^{t}Bu in THF (1 M, 0.16 mL, 0.159 mmol). The reaction mixture was stirred for 30 min. To the reaction mixture was added tert-butyl 7-(6-(2,8-dimethylimidazo[1,2-b]pyridazin-6-yl)-8-fluoro-1-oxoisoquinolin-2(1H)-yl)-4-azaspiro[2.5]octane-4-carboxylate (75 mg, 0.145 mmol). The resulting mixture was stirred at 60° C. for 3 h, then cooled to room temperature. Water (10 mL) and EtOAc (10 mL) were added to the reaction mixture, and the layers were separated. The aqueous phase was extracted with ethyl acetate (10 mL). The organic layers were combined, dried over Na₂SO₄, filtered, and the filtrate concentrated under reduced pressure to give a residue. The residue was purified by column chromatography on silica gel (4 g) using a gradient of 0-100% ethyl acetate in hexane to afford tert-butyl 7-(6-(2,8-dimethylimidazo[1,2-b]pyridazin-6-yl)-8-((4-methoxybenzyl)oxy)-1-oxoisoquinolin-2(1H)-yl)-4-azaspiro[2.5]octane-4-carboxylate (72 mg, 78%) as a liquid. LCMS (ES, m/z): 636.3 [M+H]⁺. ¹H NMR (CHCl₃-d, 400 MHz): δ_{H} 7.82 (1H, s), 7.61 (2H, d, J=8.2 Hz), 7.57 (2H, s), 7.29 (1H, s), 7.13 (1H, d, J=7.5 Hz), 6.98 (2H, d, J=8.1 Hz), 6.53 (1H, d, J=7.4 Hz), 5.54-5.51 (1H, m), 5.37 (2H, s), 4.21 (1H, d, J=13.6 Hz), 3.84 (3H, s), 3.11 (1H, t, J=12.7 Hz), 2.75 (3H, s), 2.57 (3H, s), 2.24-2.20 (1H, m), 1.96 (1H, d, J=12.2 Hz), 1.69 (1H, s), 1.54 (9H, s), 1.37 (1H, s), 1.30-1.23 (1H, m), 0.92 (1H, s), 0.73 (1H, s), 0.67 (1H, s).

### Synthesis of Compound 466

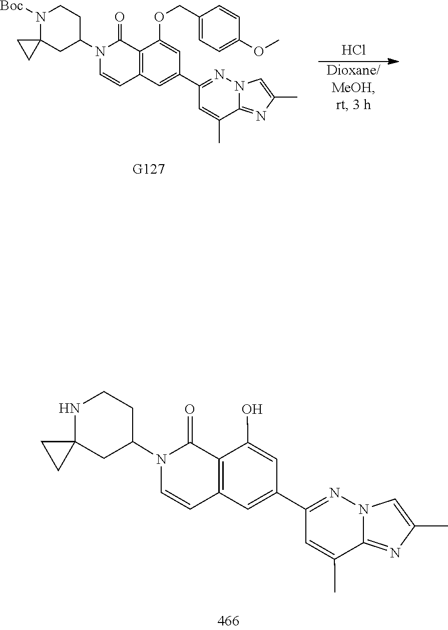 [SMILES: COc1ccc(COc2cc(-c3cc(C)c4nc(C)cn4n3)cc3ccn(C4CCN(C(=O)OC(C)(C)C)C5(CC5)C4)c(=O)c23)cc1 ; Cl ; Cc1cn2nc(-c3cc(O)c4c(=O)n(C5CCNC6(CC6)C5)ccc4c3)cc(C)c2n1]

To a solution of tert-butyl 7-(6-(2,8-dimethylimidazo[1,2-b]pyridazin-6-yl)-8-((4-methoxybenzyl)oxy)-1-oxoisoquinolin-2(1H)-yl)-4-azaspiro[2.5]octane-4-carboxylate (70 mg, 0.11 mmol) in methanol (3.0 mL) was added 4.0 M HCl solution in dioxane (1.38 mL, 5.51 mmol). The reaction mixture was stirred at room temperature for 3 h, then concentrated under reduced pressure to give a residue. DCM (10 mL) and a saturated solution of NaHCO₃ (10 mL) were added to the residue, and the layers were separated. The aqueous phase was extracted with DCM (2×10 mL). The organic layers were combined, dried over Na₂SO₄, filtered, and the filtrate concentrated under reduced pressure to give a residue. The residue was purified by column chromatography on silica gel (4 g) using a gradient of 0-20% MeOH:NH₄OH (9:1) in DCM to afford 6-(2,8-dimethylimidazo[1,2-b]pyridazin-6-yl)-8-hydroxy-2-(4-azaspiro[2.5]octan-7-yl)isoquinolin-1(2H)-one (13.5 mg, 30%) as a solid. LCMS (ES, m/z): 416.2 [M+H]⁺. ¹H NMR (CHCl₃-d, 400 MHz): δ_{H} 13.00 (1H, s), 7.78 (1H, s), 7.51 (1H, s), 7.43 (1H, s), 7.27 (1H, s), 7.16 (1H, d, J=7.6 Hz), 6.67 (1H, d, J=7.6 Hz), 5.24 (1H, t, J=12.0 Hz), 3.24 (1H, d, J=13.2 Hz), 3.02 (1H, t, J=12.6 Hz), 2.72 (3H, s), 2.54 (3H, s), 2.24 (1H, t, J=12.3 Hz), 2.02 (1H, d, J=12.5 Hz), 1.80 (1H, br s), 1.36 (1H, d, J=12.6 Hz), 0.78 (1H, s), 0.71 (1H, s), 0.59 (2H, s).

### Example G70: Synthesis of Compound 1248

### Synthesis of Intermediate G128

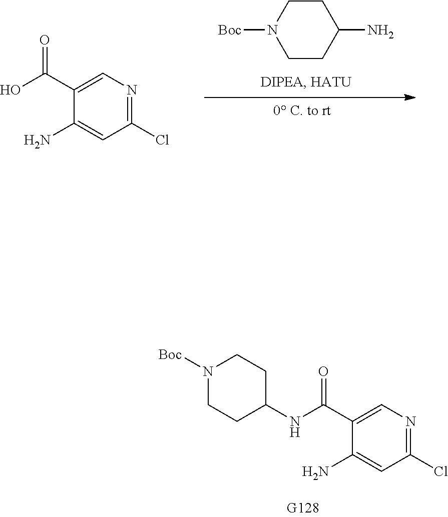 [SMILES: Nc1cc(Cl)ncc1C(=O)O ; CC(C)(C)OC(=O)N1CCC(N)CC1 ; CC(C)(C)OC(=O)N1CCC(NC(=O)c2cnc(Cl)cc2N)CC1]

To a mixture of 4-amino-6-chloronicotinic acid (1.04 g, 6.03 mmol) and tert-butyl 4-aminopiperidine-1-carboxylate in DMF (52.0 mL) at 0° C. was added DIPEA (2.62 mL, 15.1 mmol) followed by HATU (2.52 g, 6.63 mmol) in portions. The reaction mixture was stirred for 3 h, then concentrated in vacuo to give a residue. The residue was partitioned between DCM (60 mL) and saturated NaHCO₃ (60 mL), and the layers were separated. The aqueous layer extracted with DCM (3×30 mL). The organic layers were combined, washed with brine, dried over Na₂SO₄, and filtered. The filtrate was concentrated under reduced pressure to give a residue. The residue was purified by flash chromatography on a 40 g column using a gradient from 0-10% methanol in DCM to provide tert-butyl 4-(4-amino-6-chloronicotinamide)piperidine-1-carboxylate (2.06 g, 96%). LCMS (ES, m/z): 355.2 [M+H]⁺.

### Synthesis of Intermediate G129 and G130

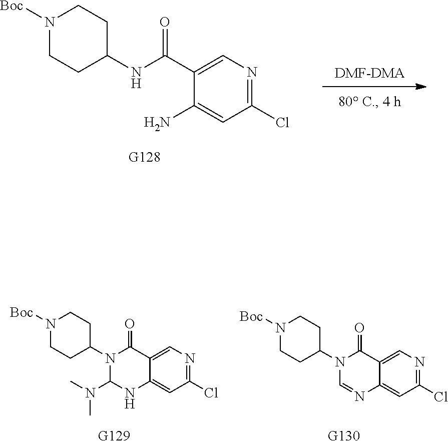

A mixture of DMF-DMA (7.49 mL, 56.4 mmol) and tert-butyl 4-(4-amino-6-chloronicotinamido)piperidine-1-carboxylate (1.00 g, 2.82 mmol) was heated at 80° C. for 4 h. The reaction mixture was diluted with ethyl acetate (100 mL), then washed with saturated NaHCO₃ (25 mL), followed by brine (25 mL). The organic phase was dried over Na₂SO₄, filtered, and the filtrate concentrated in vacuo to give a mixture of tert-butyl 4-(7-chloro-2-(dimethylamino)-4-oxo-1,4-dihydropyrido[4,3-d]pyrimidin-3(2H)-yl)piperidine-1-carboxylate and tert-butyl 4-(7-chloro-4-oxopyrido[4,3-d]pyrimidin-3(4H)-yl)piperidine-1-carboxylate. LCMS (ES, m/z): 129; 410.2 [M+H]⁺ and 130; 365.1 [M+H]⁺.

### Synthesis of Intermediate G131

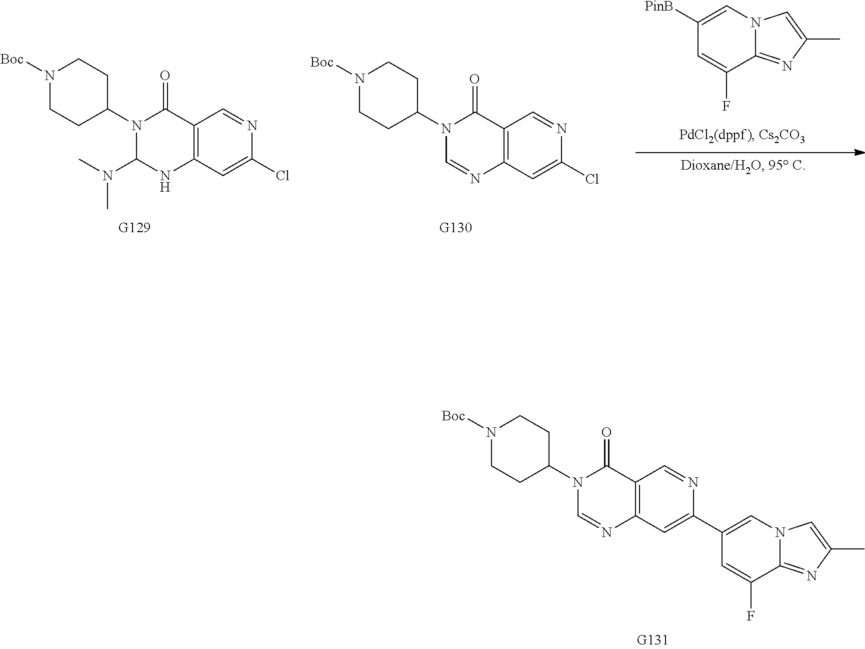

A mixture of tert-butyl 4-(7-chloro-2-(dimethylamino)-4-oxo-1,4-dihydropyrido[4,3-d]pyrimidin-3(2H)-yl)piperidine-1-carboxylate and tert-butyl 4-(7-chloro-4-oxopyrido[4,3-d]pyrimidin-3(4H)-yl)piperidine-1-carboxylate (100 mg, 0.244 mmol), 8-fluoro-2-methyl-6-(4,4,5,5-tetramethyl-1,3,2-dioxaborolan-2-yl)imidazo[1,2-a]pyridine (77.5 mg, 0.281 mmol), Cs₂CO₃ (238 mg, 0.732 mmol), and PdCl₂(dppf) (8.93 mg, 0.0122 mmol) in dioxane (2.88 mL) and water (288 uL) was purged with argon for 10 min. The reaction mixture was heated at 95° C. for 2 h under argon atmosphere, then cooled to room temperature. To the resulting mixture was added DCM (4 ml), and the reaction was stirred for an additional 1 h. The resulting mixture was filtered through celite, rinsed with DCM, and concentrated in vacuo to give a residue. The residue was partitioned between DCM (10 ml) and water (10 ml), and the organic layer separated. The aqueous layer was extracted with DCM (3×5 ml). The organic layers were combined, dried over Na₂SO₄, filtered, and the filtrate concentrated in vacuo to give a residue. The residue was purified by flash chromatography on a 24 g column using a gradient of 80-100% ethyl acetate in hexanes to afford tert-butyl 4-(7-(8-fluoro-2-methylimidazo[1,2-a]pyridin-6-yl)-4-oxopyrido[4,3-d]pyrimidin-3(4H)-yl)piperidine-1-carboxylate (76 mg, 66%) as a solid. LCMS (ES, m/z): 479.2 [M+H]⁺.

### Synthesis of Compound 1248

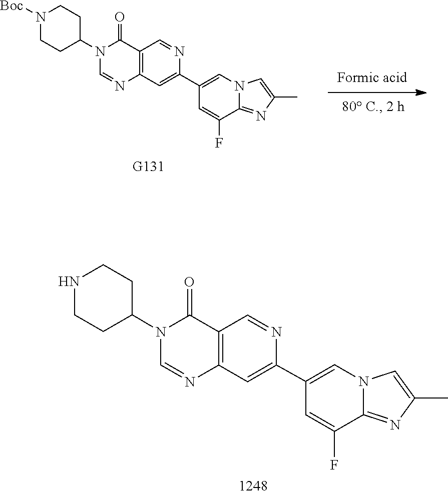 [SMILES: Cc1cn2cc(-c3cc4ncn(C5CCN(C(=O)OC(C)(C)C)CC5)c(=O)c4cn3)cc(F)c2n1 ; Cc1cn2cc(-c3cc4ncn(C5CCNCC5)c(=O)c4cn3)cc(F)c2n1]

A solution of tert-butyl 4-(7-(8-fluoro-2-methylimidazo[1,2-a]pyridin-6-yl)-4-oxopyrido[4,3-d]pyrimidin-3(4H)-yl)piperidine-1-carboxylate (76.1 mg, 0.159 mmol) in formic acid (5 mL) was heated at 80° C. for 2 h. The reaction mixture was then concentrated in vacuo to give a residue. The residue was partitioned between NaOH (0.5N, 10 mL) and DCM (20 mL). The resulting biphasic mixture was stirred vigorously for 2 h. A precipitate formed that was collected by vacuum filtration, rinsed with water, and dried under high vacuum to afford 7-(8-fluoro-2-methylimidazo[1,2-a]pyridin-6-yl)-3-(piperidin-4-yl)pyrido[4,3-d]pyrimidin-4(3)-one (25 mg, 42%) as a solid. LCMS (ES, m/z): 379.1 [M+H]⁺. ¹H NMR (DMSO-d₆, 300 MHz): δ 9.37 (1H, s), 9.35 (1H, s), 8.66 (1H, s), 8.19 (1H, s), 7.92-7.97 (2H, m), 4.59-4.70 (1H, m), 3.08 (2H, d, J=12.4 Hz), 2.60 (2H, t, J=11.8 Hz), 2.37 (3H, s), 1.84-1.98 (2H, m), 1.78 (2H, d, J=11.9 Hz).

### Example G71: Synthesis of Compound 1204

### Synthesis of Intermediate G132

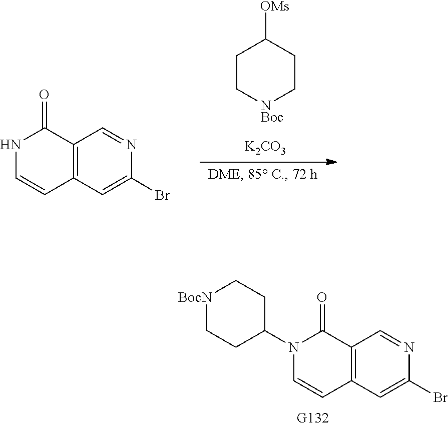 [SMILES: O=c1[nH]ccc2cc(Br)ncc12 ; "K2CO3" ; CC(C)(C)OC(=O)N1CCC(OS(C)(=O)=O)CC1 ; CC(C)(C)OC(=O)N1CCC(n2ccc3cc(Br)ncc3c2=O)CC1]

A mixture of 6-bromo-2,7-naphthyridin-1(2H)-one (0.25 g, 1.1 mmol), tert-butyl 4-((methylsulfonyl)oxy)piperidine-1-carboxylate (0.93 g, 3.3 mmol), and K₂CO₃ (0.31 g, 2.2 mmol) in DME (9.0 mL) was heated to 85° C. for 72 h, then cooled to room temperature. The reaction mixture was filtered, and the filtrate concentrated under reduced pressure to give a residue. Water (20 mL) and DCM (20 mL) were added to the residue, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-60% ethyl acetate in hexane to afford tert-butyl 4-(6-bromo-1-oxo-2,7-naphthyridin-2(1H)-yl)piperidine-1-carboxylate (0.30 g, 66%) as a solid. LCMS (ES, m/z): 408.1 [M+H]⁺.

### Synthesis of Intermediate G133

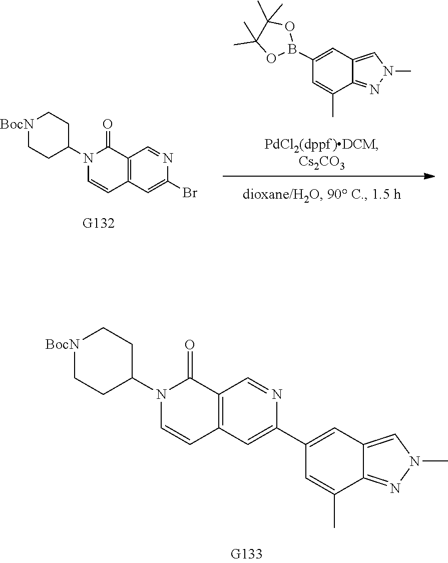

A mixture of tert-butyl 4-(6-bromo-1-oxo-2,7-naphthyridin-2(1H)-yl)piperidine-1-carboxylate (150 mg, 0.36 mmol), 2,7-dimethyl-5-(4,4,5,5-tetramethyl-1,3,2-dioxaborolan-2-yl)-2H-indazole (150 mg, 0.55 mmol), Cs₂CO₃ (299 mg, 0.92 mmol), and Pd(dppf)Cl₂·DCM (30 mg, 0.036 mmol) in a mixture of dioxane (6.0 mL) and water (0.7 mL) was heated to 90° C. for 1.5 h, then cooled to room temperature. The reaction mixture was filtered over celite using 10% methanol in DCM as eluent. The volatiles were evaporated under reduced pressure to give a residue. The residue was purified by column chromatography on silica gel using a gradient of 0-5% methanol in ethyl acetate to afford tert-butyl 4-(6-(2,7-dimethyl-2H-indazol-5-yl)-1-oxo-2,7-naphthyridin-2(1H)-yl)piperidine-1-carboxylate (125 mg, 65%) as a solid. LCMS (ES, m/z): 474.4 [M+H]⁺.

### Synthesis of Compound 1204

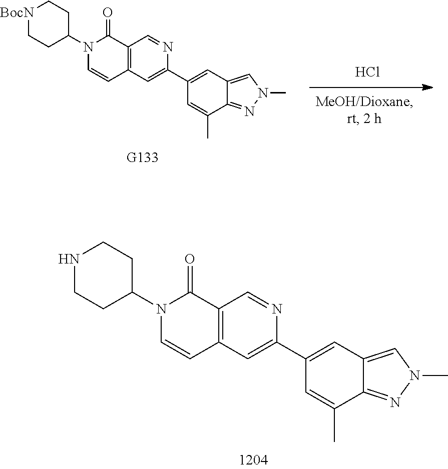 [SMILES: Cc1cc(-c2cc3ccn(C4CCN(C(=O)OC(C)(C)C)CC4)c(=O)c3cn2)cc2cn(C)nc12 ; Cl ; Cc1cc(-c2cc3ccn(C4CCNCC4)c(=O)c3cn2)cc2cn(C)nc12]

To a solution of tert-butyl 4-(6-(2,7-dimethyl-2H-indazol-5-yl)-1-oxo-2,7-naphthyridin-2(1H)-yl)piperidine-1-carboxylate (250 mg, 0.53 mmol) in methanol (12 mL) was added 4 M HCl solution in dioxane (8.0 mL, 32 mmol). The reaction mixture was stirred at room temperature for 2 h. The volatiles were evaporated under reduced pressure. An aqueous solution of NaHCO₃ (20 mL) and DCM (30 mL) were added to the resulting mixture, and the layers were separated. The aqueous layer was extracted with DCM (3×30 mL). The organic layers were combined, dried over Na₂SO₄, filtered, and the filtrate concentrated under reduced pressure to give a residue. The residue was purified by column chromatography on silica gel using a gradient of 0-20% MeOH:Et₃N (2:1 ratio) in DCM. The fractions containing product were collected and evaporated under reduced pressure to give a residue. Water (10 mL) and DCM (10 mL) were added to the residue, and the layers were separated. The aqueous layer was extracted with DCM (3×10 mL). The organic layers were combined, dried over Na₂SO₄, filtered, and the filtrate concentrated under reduced pressure to afford 6-(2,7-dimethyl-2H-indazol-5-yl)-2-(piperidin-4-yl)-2,7-naphthyridin-1(2H)-one (160 mg, 81%) as a solid. LCMS (ES, m/z): 374.2 [M+H]⁺. ¹H NMR (DMSO-d₆, 400 MHz): δ_{H} 9.39 (1H, s), 8.46 (1H, s), 8.43 (1H, s), 8.15 (1H, s), 7.88 (1H, s), 7.76 (1H, d, J=7.5 Hz), 6.70 (1H, d, J=7.5 Hz), 4.82-4.88 (1H, m), 4.20 (3H, s), 3.08 (2H, d, J=12.1 Hz), 2.64 (2H, m), 2.59 (3H, s), 1.71-1.80 (4H, m).

### Example G72: Synthesis of Compound 1198

### Synthesis of Intermediate G134

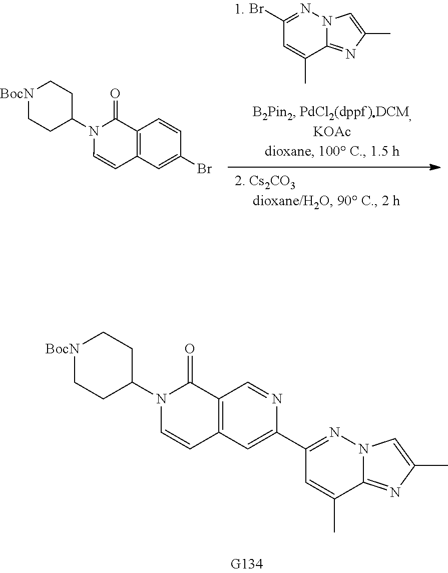 [SMILES: CC(C)(C)OC(=O)N1CCC(n2ccc3cc(Br)ccc3c2=O)CC1 ; Cc1cn2nc(Br)cc(C)c2n1 ; Cc1cn2nc(-c3cc4ccn(C5CCN(C(=O)OC(C)(C)C)CC5)c(=O)c4cn3)cc(C)c2n1]

A mixture of 6-bromo-2,8-dimethylimidazo[1,2-b]pyridazine (112 mg, 0.49 mmol), bis(pinacolato)diboron (127 mg, 0.49 mmol), PdCl₂(dppf)·DCM (27 mg, 0.036 mmol), and KOAc (109 mg, 1.1 mmol) in dioxane (2.5 mL) was heated to 100° C. for 1.5 h. To the reaction mixture was added a solution of tert-butyl 4-(6-bromo-1-oxo-2,7-naphthyridin-2(1H)-yl)piperidine-1-carboxylate (150 mg, 0.36 mmol) in dioxane (2.0 mL), followed by Cs₂CO₃ (359 mg, 1.1 mmol), then water (0.8 mL) under argon atmosphere. The reaction mixture was heated at 90° C. for 2 h, then cooled to room temperature. The resulting mixture was filtered over celite using 20% methanol in DCM as eluent. The volatiles were evaporated under reduced pressure. Water (20 mL) and DCM (20 mL) were added to the resulting mixture, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-2% methanol in ethyl acetate to afford tert-butyl 4-(6-(2,8-dimethylimidazo[1,2-b]pyridazin-6-yl)-1-oxo-2,7-naphthyridin-2(1H)-yl)piperidine-1-carboxylate (110 mg, 63%) as a solid. LCMS (ES, m/z): 475.3 [M+H]⁺.

### Synthesis of Compound 1198

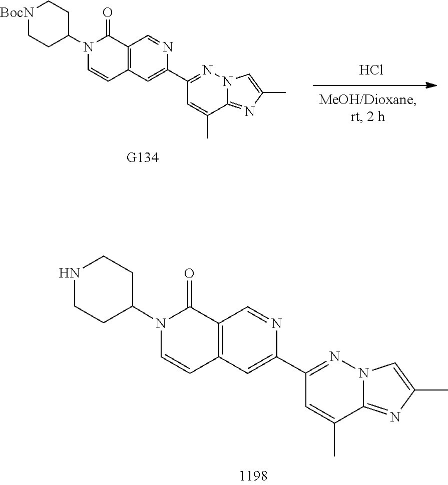 [SMILES: Cc1cn2nc(-c3cc4ccn(C5CCN(C(=O)OC(C)(C)C)CC5)c(=O)c4cn3)cc(C)c2n1 ; Cl ; Cc1cn2nc(-c3cc4ccn(C5CCNCC5)c(=O)c4cn3)cc(C)c2n1]

To a solution of tert-butyl 4-(6-(2,8-dimethylimidazo[1,2-b]pyridazin-6-yl)-1-oxo-2,7-naphthyridin-2(1H)-yl)piperidine-1-carboxylate (110 mg, 0.23 mmol) in methanol (5.0 mL) was added 4 M HCl solution in dioxane (5.0 mL, 20 mmol). The reaction mixture was stirred at room temperature for 2 h, then concentrated under reduced pressure to give a residue. An aqueous solution of NaHCO₃ (20 mL) and DCM (30 mL) were added to the residue, and the layers were separated. The aqueous layer was extracted with DCM (3×30 mL). The organic layers were combined, dried over Na₂SO₄, filtered, and the filtrate concentrated under reduced pressure to give a residue. To the residue was added acetonitrile (10 mL), and the resulting mixture was sonicated. A solid formed that was collected by filtration, washed with acetonitrile (10 mL), and dried under vacuum to afford 6-(2,8-dimethylimidazo[1,2-b]pyridazin-6-yl)-2-(piperidin-4-yl)-2,7-naphthyridin-1(2H)-one (67 mg, 77%) as a solid. LCMS (ES, m/z): 375.2 [M+H]⁺. ¹H NMR (DMSO-d₆, 400 MHz): δ_{H} 9.45 (1H, s), 8.44 (1H, s), 8.10 (1H, s), 8.04 (1H, s), 7.84 (1H, d, J=7.5 Hz), 6.87 (1H, d, J=7.5 Hz), 4.86 (1H, m), 3.09 (2H, d, J=12.1 Hz), 2.60-2.64 (5H, m), 2.43 (3H, s), 1.73-1.80 (4H, m).

### Example G73: Synthesis of Compound 1205

### Synthesis of Intermediate G135

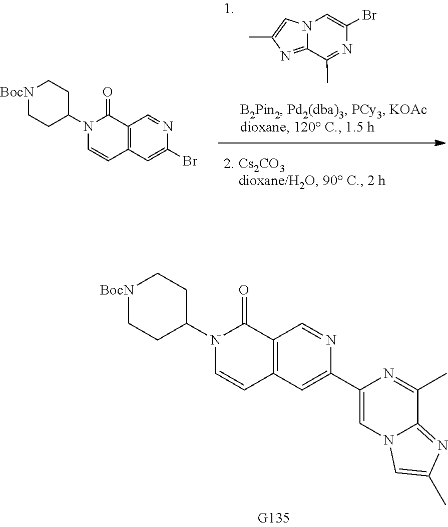 [SMILES: CC(C)(C)OC(=O)N1CCC(n2ccc3cc(Br)ncc3c2=O)CC1 ; Cc1cn2cc(Br)nc(C)c2n1 ; Cc1cn2cc(-c3cc4ccn(C5CCN(C(=O)OC(C)(C)C)CC5)c(=O)c4cn3)nc(C)c2n1]

A mixture of 6-bromo-2,8-dimethylimidazo[1,2-a]pyrazine (160 mg, 0.70 mmol), bis(pinacolato)diboron (269 mg, 1.06 mmol), KOAc (208 mg, 2.1 mmol), Pd₂(dba)₃ (129 mg, 0.14 mmol), and PCy₃ (83 mg, 0.23 mmol) in dioxane (11 mL) was heated to 120° C. for 1.5 h, then cooled to room temperature. The reaction mixture was filtered over celite using 10% methanol in DCM as eluent. The volatiles were evaporated under reduced pressure to give a residue. To the residue was added a mixture of tert-butyl 4-(6-bromo-1-oxo-2,7-naphthyridin-2(1H)-yl)piperidine-1-carboxylate (150 mg, 0.36 mmol), PdCl₂(dppf)·DCM (27 mg, 0.036 mmol), and Cs₂CO₃ (359 mg, 1.1 mmol) in a mixture of dioxane (6.0 mL) and water (0.8 mL). The resulting mixture was heated at 90° C. for 2 h, then cooled to room temperature and filtered over celite using 20% methanol in DCM as eluent. The volatiles were evaporated under reduced pressure. Water (20 mL) and DCM (20 mL) were added to the resulting mixture, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-100% ethyl acetate in hexane to afford tert-butyl 4-(6-(2,8-dimethylimidazo[1,2-a]pyrazin-6-yl)-1-oxo-2,7-naphthyridin-2(1H)-yl)piperidine-1-carboxylate (140 mg, 80%) as a solid. LCMS (ES, m/z): 474.5 [M+H]⁺.

### Synthesis of Compound 1205

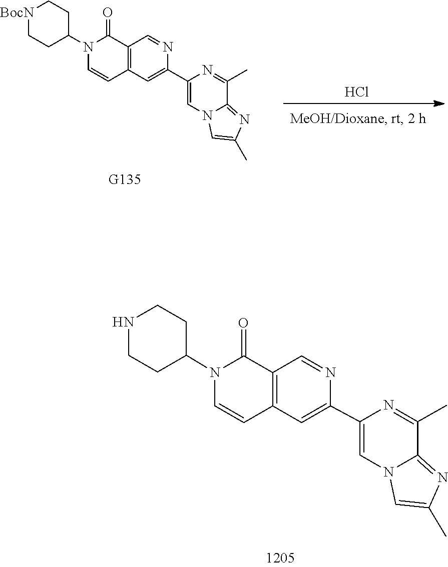 [SMILES: Cc1cn2cc(-c3cc4ccn(C5CCN(C(=O)OC(C)(C)C)CC5)c(=O)c4cn3)nc(C)c2n1 ; Cl ; Cc1cn2cc(-c3cc4ccn(C5CCNCC5)c(=O)c4cn3)nc(C)c2n1]

To a solution of tert-butyl 4-(6-(2,8-dimethylimidazo[1,2-a]pyrazin-6-yl)-1-oxo-2,7-naphthyridin-2(1H)-yl)piperidine-1-carboxylate (140 mg, 0.29 mmol) in methanol (7.0 mL) was added 4 M HCl solution in dioxane (5.0 mL, 20 mmol). The reaction mixture was stirred at room temperature for 2 h. The volatiles were evaporated under reduced pressure. An aqueous solution of NaHCO₃ (20 mL) was added to the resulting mixture. The aqueous layer was extracted with a solution of 20% ⁱPrOH in CHCl₃ (3×100 mL). The organic layers were combined, dried over Na₂SO₄, filtered, and the filtrate concentrated under reduced pressure. MeCN (10 mL) was added, and the resulting mixture was sonicated. A solid formed that was collected by filtration, washed with acetonitrile (10 mL) and dried under vacuum to afford 6-(2,8-dimethylimidazo[1,2-a]pyrazin-6-yl)-2-(piperidin-4-yl)-2,7-naphthyridin-1(2H)-one (80 mg, 72%) as a solid. LCMS (ES, m/z): 375.2 [M+H]⁺. ¹H NMR (CHCl₃-d, 400 MHz): δ_{H} 9.59 (1H, s), 9.14 (1H, s), 8.41 (1H, s), 7.52 (1H, s), 7.39 (1H, d, J=7.5 Hz), 6.62 (1H, d, J=7.5 Hz), 5.11 (1H, m), 3.26 (2H, d, J=12.2 Hz), 2.97 (3H, s), 2.87 (2H, t, J=12.0 Hz), 2.54 (3H, s), 1.95 (2H, d, J=11.9 Hz), 1.73-1.81 (2H, m).

### Example G74: Synthesis of Compound 1206

### Synthesis of Compound 1206

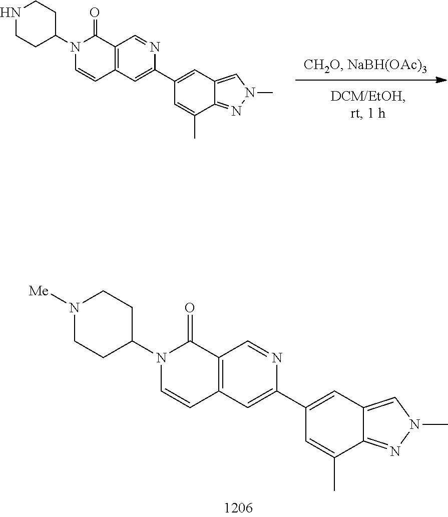 [SMILES: Cc1cc(-c2cc3ccn(C4CCNCC4)c(=O)c3cn2)cc2cn(C)nc12 ; Cc1cc(-c2cc3ccn(C4CCN(C)CC4)c(=O)c3cn2)cc2cn(C)nc12]

To a solution of 6-(2,7-dimethyl-2H-indazol-5-yl)-2-(piperidin-4-yl)-2,7-naphthyridin-1(2H)-one (35 mg, 0.094 mmol) in DCM (5.0 mL) and ethanol (1.6 mL) was added formaldehyde (37% in H₂O, 47 μL, 0.47 mmol), followed by sodium triacetoxyborohydride (119 mg, 0.56 mmol). The reaction mixture was stirred at room temperature for 1 h. The volatiles were evaporated under reduced pressure. A saturated solution of NaHCO₃ (10 mL) and DCM (10 mL) were added to the resulting mixture, and the layers were separated. The aqueous layer was extracted with DCM (3×10 mL). The organic layers were combined, dried over Na₂SO₄, filtered, and the filtrate concentrated under reduced pressure to give a residue. The residue was purified by column chromatography on silica gel using a gradient of 0-15% methanol in a mixture of DCM:NH₃ (9:1) to afford 6-(2,7-dimethyl-2H-indazol-5-yl)-2-(1-methylpiperidin-4-yl)-2,7-naphthyridin-1(2H)-one (33 mg, 91%) as a solid. LCMS (ES, m/z): 388.2 [M+H]⁺. ¹H NMR (DMSO-d₆, 400 MHz): δ_{H} 9.39 (1H, s), 8.45 (2H, d, J=12.2 Hz), 8.16 (1H, s), 7.88 (1H, s), 7.80 (1H, d, J=7.5 Hz), 6.69 (1H, d, J=7.5 Hz), 4.76 (1H, m), 4.20 (3H, s), 2.92 (2H, d, J=10.9 Hz), 2.59 (3H, s), 2.23 (3H, s), 2.07 (2H, m), 1.95 (2H, m), 1.73 (2H, d, J=11.4 Hz).

### Example G75: Synthesis of Compound 1207

### Synthesis of Compound 1207

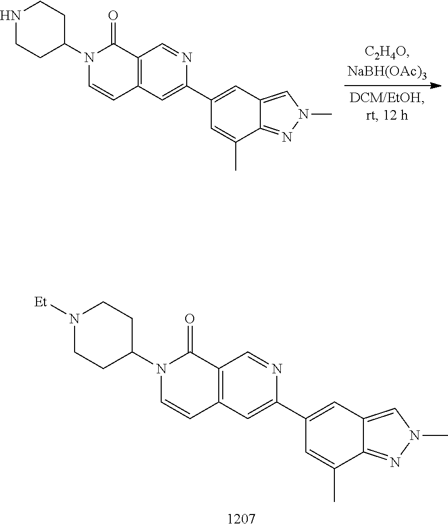 [SMILES: Cc1cc(-c2cc3ccn(C4CCNCC4)c(=O)c3cn2)cc2cn(C)nc12 ; CCN1CCC(n2ccc3cc(-c4cc(C)c5nn(C)cc5c4)ncc3c2=O)CC1]

To a mixture of 6-(2,7-dimethyl-2H-indazol-5-yl)-2-(piperidin-4-yl)-2,7-naphthyridin-1(2H)-one (30 mg, 0.080 mmol) in DCM (4.0 mL) and ethanol (1.4 mL) was added acetaldehyde (22 μL, 0.40 mmol), followed by sodium triacetoxyborohydride (102 mg, 0.48 mmol). The reaction mixture was stirred at room temperature for 12 h, then under reduced pressure. A saturated solution of NaHCO₃ (10 mL) and DCM (10 mL) were added to the resulting mixture, and the layers were separated. The aqueous layer was extracted with DCM (3×10 mL). The organic layers were combined, dried over Na₂SO₄, filtered, and the filtrate concentrated under reduced pressure to give a residue. The residue was purified by column chromatography on silica gel using a gradient of 0-12% methanol in a mixture of DCM:NH₃ (9:1) to afford 6-(2,7-dimethyl-2H-indazol-5-yl)-2-(1-ethylpiperidin-4-yl)-2,7-naphthyridin-1(2H)-one (29 mg, 91%) as a solid. LCMS (ES, m/z): 402.3 [M+H]⁺. ¹H NMR (DMSO-d₆, 400 MHz): δ_{H} 9.39 (1H, s), 8.46 (1H, s), 8.44 (s, 1H), 8.16 (1H, s), 7.88 (1H, s), 7.81 (1H, d, J=7.5 Hz), 6.69 (1H, d, J=7.5 Hz), 4.78 (1H, m), 4.20 (3H, s), 3.03 (2H, d, J=10.8 Hz), 2.59 (3H, s), 2.36-2.41 (2H, m), 2.05 (2H, m), 1.97 (2H, m), 1.75 (2H, d, J=11.3 Hz), 1.03 (3H, t, J=7.1 Hz).

### Example G76: Synthesis of Compound 1208

### Synthesis of Compound 1208

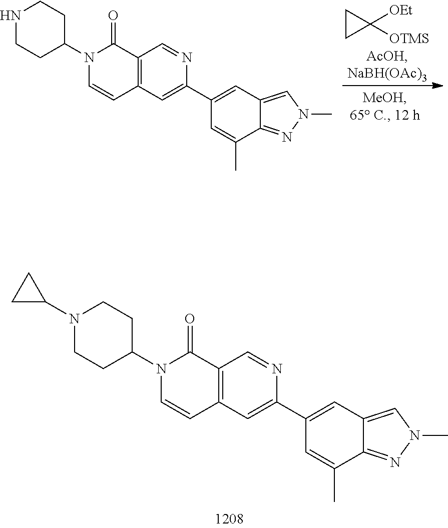

To a solution of 6-(2,7-dimethyl-2H-indazol-5-yl)-2-(piperidin-4-yl)-2,7-naphthyridin-1(2H)-one (30 mg, 0.080 mmol) in methanol (5.0 mL) was added (1-ethoxycyclopropoxy)trimethylsilane (81 μL, 0.40 mmol), followed by acetic acid (46 μL, 0.80 mmol). The reaction mixture was stirred at room temperature for 30 min. To the reaction mixture was added sodium triacetoxyborohydride (68 mg, 0.32 mmol). The reaction mixture was heated to 65° C. for 12 h, then cooled to room temperature and concentrate under reduced pressure to give a residue. A saturated solution of NaHCO₃ (10 mL) and DCM (10 mL) were added to the residue, and the layers were separated. The aqueous layer was extracted with DCM (3×10 mL). The organic layers were combined, dried over Na₂SO₄, filtered, and the filtrate concentrated under reduced pressure to give a residue. The residue was purified by column chromatography on silica gel using a gradient of 0-12% methanol in a mixture of DCM:NH₃ (9:1) to afford 2-(1-cyclopropylpiperidin-4-yl)-6-(2,7-dimethyl-2H-indazol-5-yl)-2,7-naphthyridin-1(2H)-one (14 mg, 43%) as a solid. LCMS (ES, m/z): 414.3 [M+H]⁺. ¹H NMR (DMSO-d₆, 400 MHz): δ_{H} 9.39 (1H, s), 8.46 (1H, s), 8.43 (1H, s), 8.15 (1H, s), 7.87 (1H, s), 7.79 (1H, d, J=7.5 Hz), 6.66 (1H, d, J=7.5 Hz), 4.81 (1H, m), 4.20 (3H, s), 3.09 (2H, d, J=11.2 Hz), 2.59 (3H, s), 2.36 (2H, m), 1.87 (2H, m), 1.73 (3H, m), 0.46 (2H, m), 0.33 (2H, br s).

### Example G77: Synthesis of Compound 1209

### Synthesis of Compound 1209

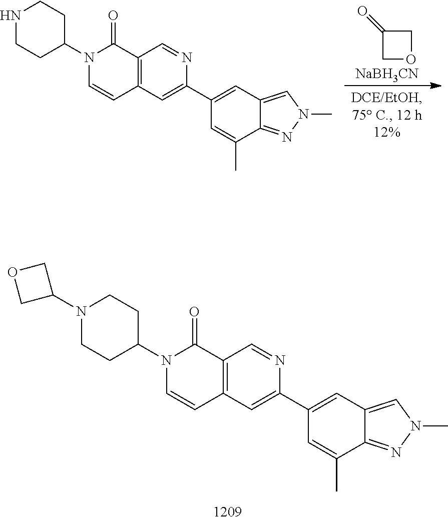

To a solution of 6-(2,7-dimethyl-2H-indazol-5-yl)-2-(piperidin-4-yl)-2,7-naphthyridin-1(2H)-one (30 mg, 0.080 mmol) in a mixture of DCE (4.0 mL) and ethanol (1.4 mL) was added 3-oxetanone (24 μL, 0.40 mmol), followed by sodium cyanoborohydride (30 mg, 0.46 mmol). The reaction mixture was stirred at 75° C. for 12 h, then cooled to room temperature. The volatiles were evaporated under reduced pressure. A saturated solution of NaHCO₃ (10 mL) and DCM (10 mL) were added to the resulting mixture, and the layers were separated. The aqueous layer was extracted with DCM (3×10 mL). The organic layers were combined, dried over Na₂SO₄, filtered, and the filtrate concentrated under reduced pressure to give a residue. The residue was purified by column chromatography on silica gel using a gradient of 0-10% methanol in a mixture of DCM:NH₃ (9:1) to afford 6-(2,7-dimethyl-2H-indazol-5-yl)-2-(1-(oxetan-3-yl)piperidin-4-yl)-2,7-naphthyridin-1(2H)-one-1 (4.0 mg, 12%) as a solid. LCMS (ES, m/z): 430.3 [M+H]⁺. ¹H NMR (DMSO-d₆, 400 MHz): δ_{H} 9.39 (1H, s), 8.47 (1H, s), 8.43 (1H, s), 8.16 (1H, s), 7.88 (1H, s), 7.84 (1H, d, J=7.5 Hz), 6.69 (1H, d, J=7.5 Hz), 4.77-4.85 (1H, m), 4.56 (2H, t, J=6.5 Hz), 4.45 (2H, t, J=6.1 Hz), 4.20 (3H, s), 3.48 (1H, m), 2.87 (2H, d, J=7.5 Hz), 2.59 (3H, s), 1.96-2.05 (4H, m), 1.77 (2H, d, J=8.9 Hz).

### Example G78: Synthesis of Compound 1210

### Synthesis of Intermediate G136

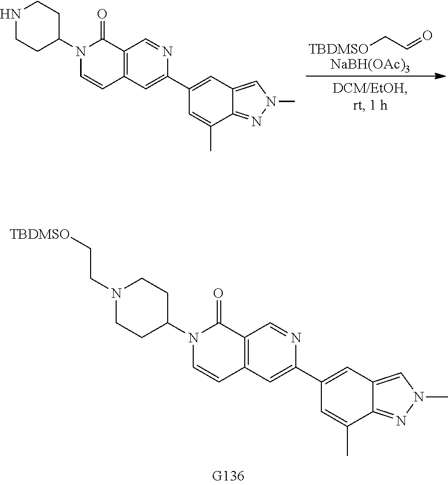 [SMILES: Cc1cc(-c2cc3ccn(C4CCNCC4)c(=O)c3cn2)cc2cn(C)nc12 ; CC(C)(C)[Si](C)(C)OCC=O ; Cc1cc(-c2cc3ccn(C4CCN(CCO[Si](C)(C)C(C)(C)C)CC4)c(=O)c3cn2)cc2cn(C)nc12 ; "NaBH(OAc)3"]

To a solution of 6-(2,7-dimethyl-2H-indazol-5-yl)-2-(piperidin-4-yl)-2,7-naphthyridin-1(2H)-one (30 mg, 0.080 mmol) in a mixture of DCM (4.0 mL) and ethanol (1.4 mL) was added (tert-butyldimethylsilyloxy)acetaldehyde (70 mg, 0.402 mmol), followed by sodium triacetoxyborohydride (102 mg, 0.48 mmol). The reaction mixture was stirred at room temperature for 1 h, then concentrated under reduced pressure. A saturated solution of NaHCO₃ (10 mL) and DCM (10 mL) were added to the resulting mixture, and the layers were separated. The organic layers were combined, dried over Na₂SO₄, filtered, and the filtrate concentrated under reduced pressure to afford 2-(1-(2-((tert-butyldimethylsilyl)oxy)ethyl)piperidin-4-yl)-6-(2,7-dimethyl-2H-indazol-5-yl)-2,7-naphthyridin-1(2H)-one. LCMS (ES, m/z): 532.3 [M+H]⁺.

### Synthesis of Compound 1210

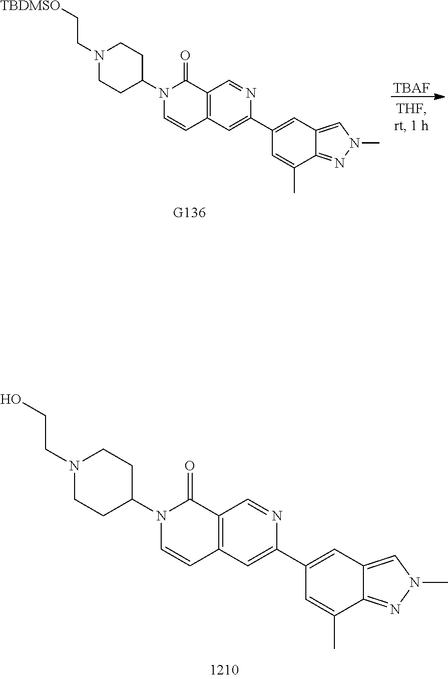 [SMILES: Cc1cc(-c2cc3ccn(C4CCN(CCO[Si](C)(C)C(C)(C)C)CC4)c(=O)c3cn2)cc2cn(C)nc12 ; "TBAF" ; Cc1cc(-c2cc3ccn(C4CCN(CCO)CC4)c(=O)c3cn2)cc2cn(C)nc12]

To a solution of 2-(1-(2-((tert-butyldimethylsilyl)oxy)ethyl)piperidin-4-yl)-6-(2,7-dimethyl-2H-indazol-5-yl)-2,7-naphthyridin-1(2H)-one in THF (1.0 mL) was added 1 M TBAF solution in THE (0.56 mL, 0.56 mmol). The reaction mixture was stirred at room temperature for 1 h, then concentrated under reduced pressure. Water (20 mL) and DCM (20 mL) were added to the resulting mixture, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-16% methanol in DCM to afford 6-(2.7-dimethyl-2H-indazol-5-yl)-2-(1-(2-hydroxyethyl)piperidin-4-yl)-2,7-naphthyridin-1(2H)-one (23 mg, 69%) as a solid. LCMS (ES, m/z): 418.3 [M+H]. ¹H NMR (DMSO-d₆, 400 MHz)): δ_{H} 9.39 (1H, s), 8.46 (1H, s), 8.43 (1H, s), 8.16 (1H, s), 7.88 (1H, s), 7.80 (1H, d, J=7.5 Hz), 6.69 (1H, d, J=7.5 Hz), 4.78 (1H, m), 4.42 (1H, br s), 4.20 (3H, s), 3.53 (2H, q, J=6 Hz), 3.29 (2H, s), 3.05 (2H, d), 2.59 (3H, s), 2.17-2.22 (2H, m), 1.94 (2H, m), 1.74 (2H, d, J=11.4 Hz).

### Example G79: Synthesis of Compound 1212

### Synthesis of Intermediate G137

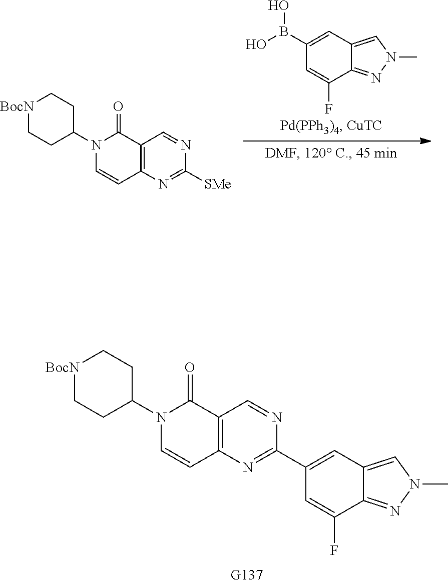 [SMILES: CSc1ncc2c(=O)n(C3CCN(C(=O)OC(C)(C)C)CC3)ccc2n1 ; Cn1cc2cc(B(O)O)cc(F)c2n1 ; Cn1cc2cc(-c3ncc4c(=O)n(C5CCN(C(=O)OC(C)(C)C)CC5)ccc4n3)cc(F)c2n1]

A mixture of tert-butyl 4-(2-(methylthio)-5-oxopyrido[4,3-d]pyrimidin-6(5H)-yl)piperidine-1-carboxylate (100 mg, 0.26 mmol), (7-fluoro-2-methyl-2H-indazol-5-yl)boronic acid (103 mg, 0.53 mmol), CuTC (152 mg, 0.79 mmol), and Pd(PPh₃)₄(31 mg, 0.026 mmol) in DMF (5.0 mL) was heated to 120° C. for 45 min, then cooled to room temperature. The volatiles were evaporated under reduced pressure. A saturated solution of NaHCO₃ (30 mL) and DCM (30 mL) were added to the resulting mixture, and the layers were separated. The aqueous layer was extracted with DCM (3×30 mL). The organic layers were combined, dried over Na₂SO₄, filtered, and the filtrate was concentrated under reduced pressure to give a residue. The residue was purified by column chromatography on silica gel using a gradient of 0-100% ethyl acetate in hexane to afford tert-butyl 4-(2-(7-fluoro-2-methyl-2H-indazol-5-yl)-5-oxopyrido[4,3-d]pyrimidin-6(5H)-yl)piperidine-1-carboxylate (107 mg, 84%) as a solid. LCMS (ES, m/z): 479.2 [M+H]⁺.

### Synthesis of Compound 1212

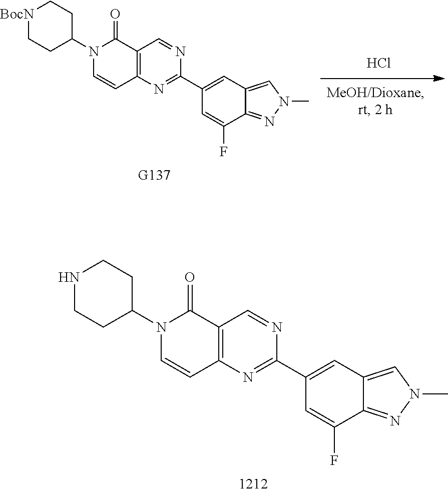 [SMILES: Cn1cc2cc(-c3ncc4c(=O)n(C5CCN(C(=O)OC(C)(C)C)CC5)ccc4n3)cc(F)c2n1 ; Cl ; Cn1cc2cc(-c3ncc4c(=O)n(C5CCNCC5)ccc4n3)cc(F)c2n1]

To a solution of tert-butyl 4-(2-(7-fluoro-2-methyl-2H-indazol-5-yl)-5-oxopyrido[4,3-d]pyrimidin-6(5H)-yl)piperidine-1-carboxylate (107 mg, 0.22 umol) in methanol (6.0 mL) was added 4 M HCl solution in dioxane (6.0 mL, 24 mmol). The reaction mixture was stirred at room temperature for 2 h, then concentrated under reduced pressure. A saturated solution of NaHCO₃ (30 mL), NaOH (0.1 M, 20 mL) and DCM (30 mL) were added to the resulting mixture, and the layers were separated. The aqueous layer was extracted with a solution of 15% isopropanol in CHCl₃ (3×50 mL). The organic layers were combined, dried over Na₂SO₄, filtered, and the filtrate concentrated under reduced pressure to give a residue. The residue was purified by column chromatography using a gradient of 0-10% methanol in a mixture of DCM:NH₃ (90:10) to afford 2-(7-fluoro-2-methyl-2H-indazol-5-yl)-6-(piperidin-4-yl)pyrido[4,3-d]pyrimidin-5(6H)-one (51 mg, 60%) as a solid. LCMS (ES, m/z): 379.2 [M+H]⁺. ¹H NMR (DMSO-d₆, 400 MHz): δ_{H} 9.52 (1H, s), 8.84 (1H, s), 8.71 (1H, d, J=2.6 Hz), 8.03-8.07 (2H, m), 6.73 (1H, d, J=7.6 Hz), 4.81 (1H, m), 4.24 (3H, s), 3.08 (2H, d, J=12.0 Hz), 2.62 (2H, m), 1.73-1.78 (4H, m).

### Example G80: Synthesis of Compound 1213

### Synthesis of Intermediate G138

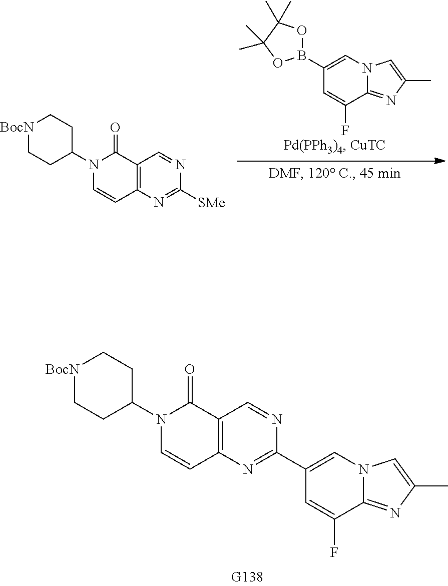

A mixture of tert-butyl 4-(2-(methylthio)-5-oxopyrido[4,3-d]pyrimidin-6(5H)-yl)piperidine-1-carboxylate (100 mg, 0.26 mmol), 8-fluoro-2-methyl-6-(4,4,5,5-tetramethyl-1,3,2-dioxaborolan-2-yl)imidazo[1,2-a]pyridine (220 mg, 0.79 mmol), CuTC (152 mg, 0.79 mmol), and Pd(PPh₃)₄(31 mg, 0.026 mmol) in DMF (5.0 mL) was heated to 120° C. for 45 min, then cooled to room temperature. The volatiles were evaporated under reduced pressure. A saturated solution of NaHCO₃ (30 mL) and DCM (30 mL) were added to the resulting mixture, and the layers were separated. The aqueous layer was extracted with DCM (3×30 mL). The organic layers were combined, dried over Na₂SO₄, filtered, and the filtrate concentrated under reduced pressure to give a residue. The residue was purified by column chromatography on silica gel using a gradient of ethyl acetate in hexane to afford tert-butyl 4-(2-(8-fluoro-2-methylimidazo[1,2-a]pyridin-6-yl)-5-oxopyrido[4,3-d]pyrimidin-6(5H)-yl)piperidine-1-carboxylate (60 mg, 47%) as a solid. LCMS (ES, m/z): 479.2 [M+H]⁺.

### Synthesis of Compound 1213

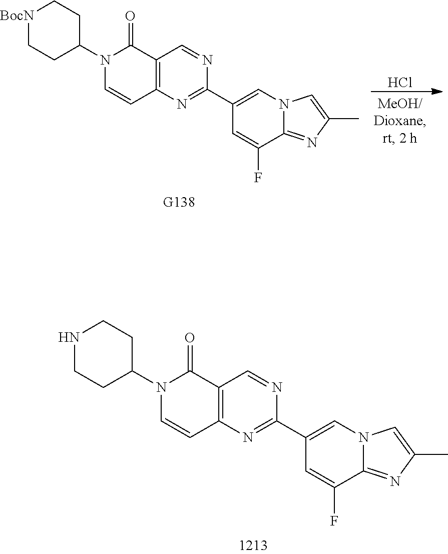 [SMILES: Cc1cn2cc(-c3ncc4c(=O)n(C5CCN(C(=O)OC(C)(C)C)CC5)ccc4n3)cc(F)c2n1 ; Cl ; Cc1cn2cc(-c3ncc4c(=O)n(C5CCNCC5)ccc4n3)cc(F)c2n1]

To a solution of tert-butyl 4-(2-(8-fluoro-2-methylimidazo[1,2-a]pyridin-6-yl)-5-oxopyrido[4,3-d]pyrimidin-6(5H)-yl)piperidine-1-carboxylate (60 mg, 0.12 mmol) in methanol (3.5 mL) was added 4 M HCl solution in dioxane (3.5 mL, 14 mmol). The reaction mixture was stirred at room temperature for 2 h. The volatiles were evaporated under reduced pressure. A saturated solution of NaHCO₃ (30 mL), NaOH (0.1 M, 20 mL) and DCM (30 mL) were added to the resulting mixture, and the layers were separated. The aqueous layer was then extracted with a solution of 15% isopropanol in CHCl₃ (3×50 mL). The organic layers were combined, dried over Na₂SO₄, filtered, and the filtrate concentrated under reduced pressure to give a residue. The residue was purified by column chromatography using a gradient of 0-10% methanol in a mixture of DCM:NH₃ (90:10) to afford 2-(8-fluoro-2-methylimidazo[1,2-a]pyridin-6-yl)-6-(piperidin-4-yl)pyrido[4,3-d]pyrimidin-5(6H)-one (10 mg, 21%) as a solid. LCMS (ES, m/z): 379.2 [M+H]⁺. ¹H NMR (DMSO-d₆, 400 MHz): δ_{H} 9.53 (2H, s), 8.09 (1H, d, J=7.8 Hz), 8.06 (1H, s), 7.93 (1H, d, J=12.3 Hz), 6.73 (1H, d, J=7.7 Hz), 4.81 (1H, m), 3.10 (2H, d, J=12.3 Hz), 2.65 (2H, m), 2.39 (3H, s), 1.74-1.79 (4H, m).

### Example G80: Synthesis of Compound 1230

### Synthesis of Intermediate G139

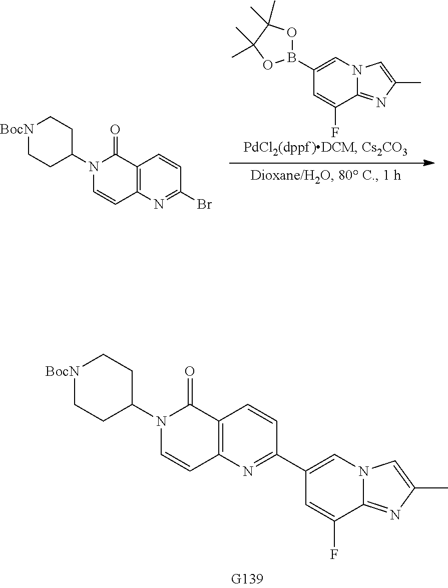

A mixture of tert-butyl 4-(2-bromo-5-oxo-1,6-naphthyridin-6(5H)-yl)piperidine-1-carboxylate (100 mg, 0.245 mmol), 8-fluoro-2-methyl-6-(4,4,5,5-tetramethyl-1,3,2-dioxaborolan-2-yl)imidazo[1,2-a]pyridine (94 mg, 0.34 mmol), Cs₂CO₃ (200 mg, 0.61 mmol), and PdCl₂(dppf)·DCM (18 mg, 0.0245 mmol) was suspended in a mixture of dioxane (7.0 mL) and water (0.8 mL). The reaction mixture was heated to 80° C. for 1 h, then cooled to room temperature and filtered over celite using 10% methanol in DCM as eluent. The volatiles were evaporated under reduced pressure to give a residue. The residue was purified by reverse phase chromatography (Condition G4, Gradient 1). The fractions containing product were collected and concentrated under reduced pressure. A saturated solution of NaHCO₃ (15 mL) and DCM (15 mL) were added to the resulting mixture, and the layers were separated. The aqueous layer was extracted with DCM (3×15 mL). The organic layers were combined, dried over Na₂SO₄, filtered, and the filtrate concentrated under reduced pressure to afford tert-butyl 4-(2-(8-fluoro-2-methylimidazo[1,2-a]pyridin-6-yl)-5-oxo-1,6-naphthyridin-6(5H)-yl)piperidine-1-carboxylate (93 mg, 79%) as a solid. LCMS (ES, m/z): 478.2 [M+H]⁺.

### Synthesis of Compound 1230

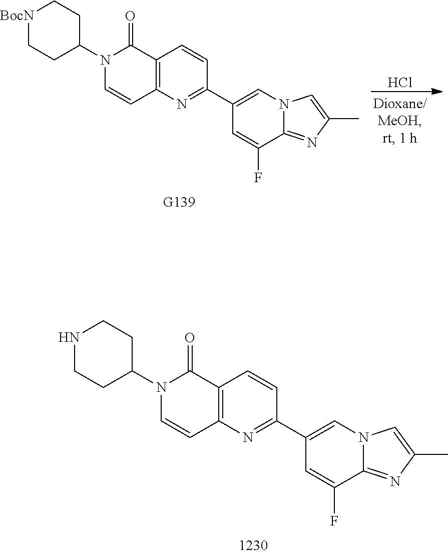 [SMILES: Cc1cn2cc(-c3ccc4c(=O)n(C5CCN(C(=O)OC(C)(C)C)CC5)ccc4n3)cc(F)c2n1 ; Cl ; Cc1cn2cc(-c3ccc4c(=O)n(C5CCNCC5)ccc4n3)cc(F)c2n1]

To a solution of tert-butyl 4-(2-(8-fluoro-2-methylimidazo[1,2-a]pyridin-6-yl)-5-oxo-1,6-naphthyridin-6(5H)-yl)piperidine-1-carboxylate (93 mg, 0.19 mmol) in methanol (5.0 mL) was added 4 M HCl solution in dioxane (3.0 mL, 12 mmol). The reaction mixture was stirred at room temperature for 1 h. The volatiles were evaporated under reduced pressure. An aqueous solution of NaHCO₃ (10 mL) and DCM (20 mL) were added to the resulting mixture, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-8% methanol in a mixture of DCM/NH₃ (9:1) to afford 2-(8-fluoro-2-methylimidazo[1,2-a]pyridin-6-yl)-6-(piperidin-4-yl)-1,6-naphthyridin-5(6H)-one (51 mg, 70%) as a solid. LCMS (ES, m/z): 378.2 [M+H]⁺. ¹H NMR (DMSO-d₆, 400 MHz): δ_{H} 9.34 (1H, s), 8.62 (1H, d, J=8.5 Hz), 8.11 (1H, d, J=8.5 Hz), 7.90-7.95 (2H, m), 7.76 (1H, d, J=7.8 Hz), 6.81 (1H, d, J=7.7 Hz), 4.91 (1H, m), 3.21 (2H, d, J=12.7 Hz), 2.81 (2H, t, J=12.1 Hz), 2.39 (3H, s), 1.80-1.98 (4H, m).

### Example G81: Synthesis of Compound 1238

### Synthesis of Intermediate G140

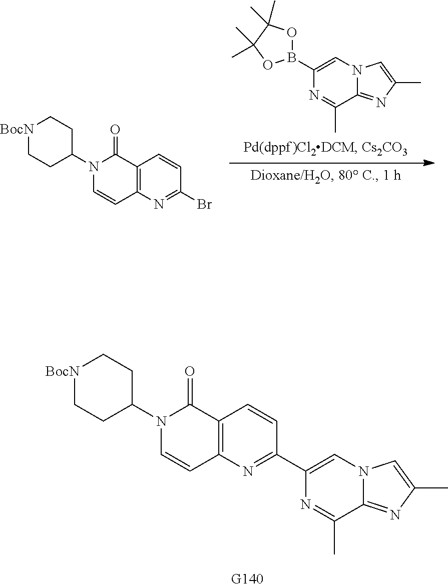

A mixture of tert-butyl 4-(2-bromo-5-oxo-1,6-naphthyridin-6(5H)-yl)piperidine-1-carboxylate (100 mg, 0.245 mmol), 2,8-dimethyl-6-(4,4,5,5-tetramethyl-1,3,2-dioxaborolan-2-yl)imidazo[1,2-a]pyrazine (94 mg, 0.34 mmol), Cs₂CO₃ (200 mg, 0.61 mmol), and PdCl₂(dppf)·DCM (18 mg, 0.0245 mmol) were suspended in a mixture of dioxane (7.0 mL) and water (0.8 mL). The reaction mixture was heated to 80° C. for 1 h, then cooled to room temperature and filtered over celite using 10% methanol in DCM as eluent. The volatiles were evaporated under reduced pressure to give a residue. The residue was purified by reverse phase chromatography (Condition G4, Gradient 1). The fractions containing product were collected and concentrated under reduced pressure. A saturated solution of NaHCO₃ (15 mL) and DCM (15 mL) were added to the resulting mixture, and the layers were separated. The aqueous layer was extracted with DCM (3×15 mL). The organic layers were combined, dried over Na₂SO₄, filtered, and the filtrate concentrated under reduced pressure to afford tert-butyl 4-(2-(2,8-dimethylimidazo[1,2-a]pyrazin-6-yl)-5-oxo-1,6-naphthyridin-6(5H)-yl)piperidine-1-carboxylate (100 mg, 86%) as a solid. LCMS (ES, m/z): 475.3 [M+H]⁺.

### Synthesis of Compound 1238

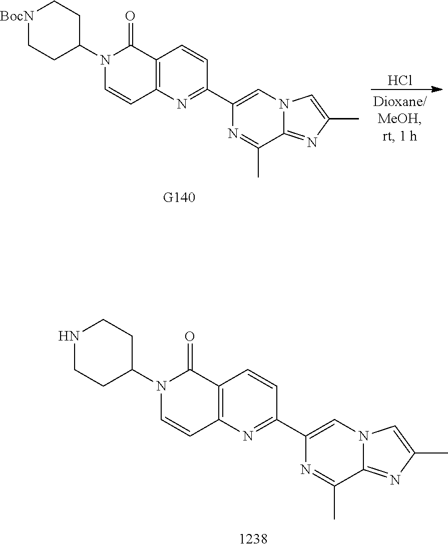 [SMILES: Cc1cn2cc(-c3ccc4c(=O)n(C5CCN(C(=O)OC(C)(C)C)CC5)ccc4n3)nc(C)c2n1 ; Cl ; Cc1cn2cc(-c3ccc4c(=O)n(C5CCNCC5)ccc4n3)nc(C)c2n1]

To a solution of tert-butyl 4-(2-(2,8-dimethylimidazo[1,2-a]pyrazin-6-yl)-5-oxo-1,6-naphthyridin-6(5H)-yl)piperidine-1-carboxylate (100 mg, 0.21 mmol) in methanol (2.5 mL) was added 4 M HCl solution in dioxane (1.6 mL, 6.4 mmol). The reaction mixture was stirred at room temperature for 1 h. The volatiles were evaporated under reduced pressure. An aqueous solution of NaHCO₃ (10 mL) and DCM (20 mL) were added to the resulting mixture, and the layers were separated. The aqueous layer was extracted with DCM (3×20 mL). The organic layers were combined, dried over Na₂SO₄, filtered, and the filtrate concentrated under reduced pressure to give a residue. The residue was suspended in DCM and filtered to afford 2-(2,8-dimethylimidazo[1,2-a]pyrazin-6-yl)-6-(piperidin-4-yl)-1,6-naphthyridin-5(6H)-one (27 mg, 34%) as a solid. LCMS (ES, m/z): 375.3 [M+H]^{+ 1}H NMR (DMSO-d₆, 400 MHz): δ_{H} 9.43 (1H, s), 8.68 (1H, d, J=8.5 Hz), 8.45 (1H, d, J=8.5 Hz), 8.07 (1H, s), 7.66 (1H, d, J=7.8 Hz), 6.82 (1H, d, J=7.7 Hz), 5.07 (1H, m), 3.41 (2H, d, J=13.0 Hz), 3.12 (2H, t, J=12.6 Hz), 2.82 (3H, s), 2.43 (3H, s), 2.22 (2H, m), 1.97 (2H, d, J=12.6 Hz).

### Example G82: Synthesis of Compound 1241

### Synthesis of Intermediate G141

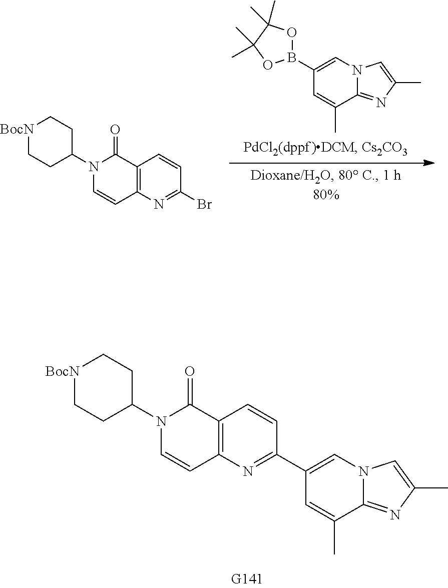

A mixture of tert-butyl 4-(2-bromo-5-oxo-1,6-naphthyridin-6(5H)-yl)piperidine-1-carboxylate (80 mg, 0.196 mmol), 2,8-dimethyl-6-(4,4,5,5-tetramethyl-1,3,2-dioxaborolan-2-yl)imidazo[1,2-a]pyridine (75 mg, 0.27 mmol), Cs₂CO₃ (160 mg, 0.49 mmol), and PdCl₂(dppf)·DCM (14 mg, 0.019 mmol) were suspended in a mixture of dioxane (6.0 mL) and water (0.7 mL). The reaction mixture was heated to 80° C. for 1 h, then cooled to room temperature and filtered over celite using 10% methanol in DCM as eluent. The volatiles were evaporated under reduced pressure to give a residue. The residue was purified by reverse phase chromatography (Condition G4, Gradient 2). The fractions containing product were collected and concentrated under reduced pressure to give a residue. A saturated solution of NaHCO₃ (15 mL) and DCM (15 mL) were added to the residue, and the layers were separated. The aqueous layer was extracted with DCM (3×15 mL). The organic layers were combined, dried over Na₂SO₄, filtered, and the filtrate concentrated under reduced pressure to afford tert-butyl 4-(2-(2,8-dimethylimidazo[1,2-a]pyridin-6-yl)-5-oxo-1,6-naphthyridin-6(5H)-yl)piperidine-1-carboxylate (75 mg, 80%) as a solid. LCMS (ES, m/z): 474.4 [M+H]⁺.

### Synthesis of Compound 1241

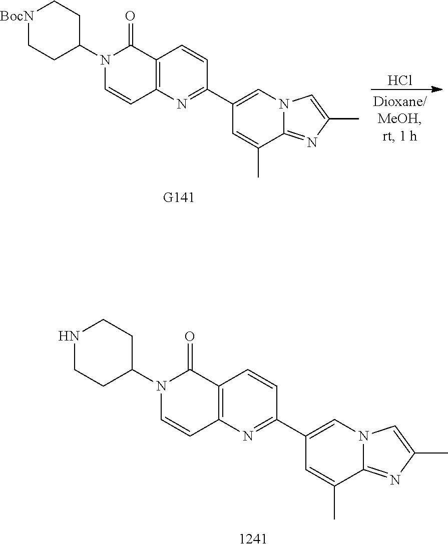 [SMILES: Cc1cn2cc(-c3ccc4c(=O)n(C5CCN(C(=O)OC(C)(C)C)CC5)ccc4n3)cc(C)c2n1 ; Cl ; Cc1cn2cc(-c3ccc4c(=O)n(C5CCNCC5)ccc4n3)cc(C)c2n1]

To a solution of tert-butyl 4-(2-(2,8-dimethylimidazo[1,2-a]pyridin-6-yl)-5-oxo-1,6-naphthyridin-6(5H)-yl)piperidine-1-carboxylate (75 mg, 0.16 mmol) in methanol (3.8 mL) was added 4 M HCl solution in dioxane (2.4 mL, 9.6 mmol). The reaction mixture was stirred at room temperature for 1 h. The volatiles were evaporated under reduced pressure. An aqueous solution of NaHCO₃ (10 mL) and DCM (20 mL) were added to the resulting mixture, and the layers were separated. The aqueous layer was extracted with a solution of 20% isopropanol in CHCl₃ (3×20 mL). The organic layers were combined, dried over Na₂SO₄, filtered, and the filtrate concentrated under reduced pressure to give a residue. The residue was purified by column chromatography on silica gel using a gradient of 0-11% methanol in DCM/NH₃ (9:1) to afford 2-(2,8-dimethylimidazo[1,2-a]pyridin-6-yl)-6-(piperidin-4-yl)-1,6-naphthyridin-5(6H)-one (27 mg, 45%) as a solid. LCMS (ES, m/z): 374.3 [M+H]⁺. ¹H NMR (DMSO-d₆, 400 MHz): δ_{H} 9.25 (1H, s), 8.58 (1H, d, J=8.5 Hz), 8.05 (1H, d, J=8.6 Hz), 7.85 (1H, s), 7.78 (2H, m), 6.77 (1H, d, J=7.7 Hz), 4.84 (1H, m), 3.10 (2H, d, J=12.2 Hz), 2.65 (2H, t, J=11.8 Hz), 2.53 (3H, s), 2.35 (3H, s), 1.73-1.81 (4H, m).

### Example G83: Synthesis of Compound 1239

### Synthesis of Intermediate G142

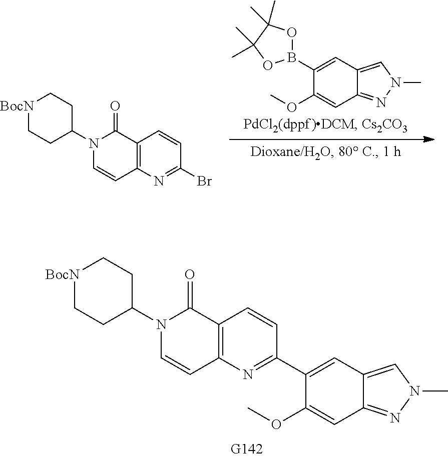

A mixture of tert-butyl 4-(2-bromo-5-oxo-1,6-naphthyridin-6(5H)-yl)piperidine-1-carboxylate (100 mg, 0.245 mmol), 6-methoxy-2-methyl-5-(4,4,5,5-tetramethyl-1,3,2-dioxaborolan-2-yl)-2H-indazole (99 mg, 0.34 mmol), Cs₂CO₃ (200 mg, 0.61 mmol), and PdCl₂(dppf)·DCM (18 mg, 0.0245 mmol) were suspended in a mixture of dioxane (7.0 mL) and water (0.8 mL). The reaction mixture was heated to 80° C. for 1 h, then cooled to room temperature and filtered over celite using 10% methanol in DCM as eluent. The volatiles were evaporated under reduced pressure to give a residue. The residue was purified by reverse phase chromatography (Condition G4, Gradient 1). The fractions containing product were collected and concentrated under reduced pressure to give a residue. A saturated solution of NaHCO₃ (15 mL) and DCM (15 mL) were added to the residue and the layers were separated. The aqueous layer was extracted with DCM (3×15 mL). The organic layers were combined, dried over Na₂SO₄, filtered, and concentrated under reduced pressure to afford tert-butyl 4-(2-(6-methoxy-2-methyl-2H-indazol-5-yl)-5-oxo-1,6-naphthyridin-6(5H)-yl)piperidine-1-carboxylate (95 mg, 79%) as a solid. LCMS (ES, m/z): 490.4 [M+H]⁺.

### Synthesis of Compound 1239

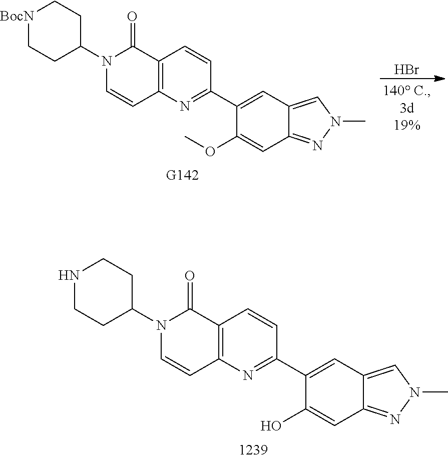 [SMILES: COc1cc2nn(C)cc2cc1-c1ccc2c(=O)n(C3CCN(C(=O)OC(C)(C)C)CC3)ccc2n1 ; Br ; Cn1cc2cc(-c3ccc4c(=O)n(C5CCNCC5)ccc4n3)c(O)cc2n1]

A mixture of tert-butyl 4-(2-(6-methoxy-2-methyl-2H-indazol-5-yl)-5-oxo-1,6-naphthyridin-6(5H)-yl)piperidine-1-carboxylate (95 mg, 0.24 mmol) and HBr (2.0 mL of 48% w/v, 11.9 mmol) was heated to 140° C. for 3 days, then cooled to room temperature. The volatiles were evaporated under reduced pressure to give a residue. A solution of 10% MeOH in DCM/NH₃ (9:1) was added to the residue. The resulting mixture was sonicated and concentrated under reduced pressure to give a residue. The residue was purified by reverse phase chromatography (Condition G5, Gradient 1) to afford 2-(6-hydroxy-2-methyl-2H-indazol-5-yl)-6-(piperidin-4-yl)-1,6-naphthyridin-5(6H)-one (20 mg, 19%) as a solid. LCMS (ES, m/z): 376.2 [M+H]⁺. ¹H NMR (DMSO-d₆, 400 MHz): δ_{H} 13.33-13.43 (1H, br s), 8.60-8.64 (2H, m), 8.42 (1H, s), 8.32 (d, 1H, J=8.9 Hz), 8.30 (2H, br s), 7.82 (1H, d, J=7.1 Hz), 6.90 (1H, s), 6.83 (1H, d, J=6.9 Hz), 4.93 (1H, m), 4.10 (3H, s), 3.22 (2H, d, J=11.2), 2.80 (2H, m), 1.95-1.81 (4H, m).

### Example G84: Synthesis of Compound 610

### Synthesis of Intermediate G143

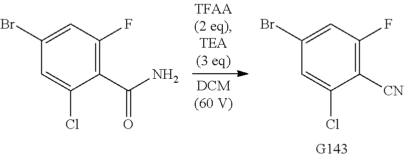 [SMILES: NC(=O)c1c(F)cc(Br)cc1Cl ; N#Cc1c(F)cc(Br)cc1Cl]

To a solution of 4-bromo-2-chloro-6-fluoro-benzamide (1.30 g, 5.15 mmol, 1.00 eq) in dichloromethane (78 mL) was added triethylamine (1.56 g, 15.45 mmol, 2.15 mL, 3.00 eq) and trifluoroacetic anhydride (2.16 g, 10.30 mmol, 1.43 mL, 2.00 eq) at 0° C. The reaction mixture was stirred at 0° C. for 0.5 h. Water (100 mL) was added to the mixture at 0° C. The resulting mixture was extracted with dichloromethane (4×50.0 mL). The organic layers were combined, dried over anhydrous Na₂SO₄, filtered, and the filtrate concentrated to give a residue, which was purified by silica gel column chromatography, eluted with petroleum ether/ethyl acetate (5:1) to give 4-bromo-2-chloro-6-fluoro-benzonitrile (800 mg, 66%) as a solid. LCMS: (ESI, m/z): 425.2 [M+H]⁺. ¹H NMR (400 MHz, DMSO-d₆) δ ppm 8.00-8.07 (m, 2H). ¹⁹F NMR (376 MHz, DMSO-d₆) δ ppm−102.396 (s, 1F).

### Synthesis of Intermediate G144

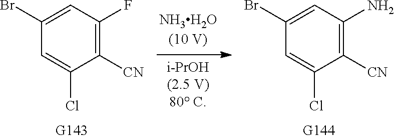 [SMILES: N#Cc1c(F)cc(Br)cc1Cl ; N#Cc1c(N)cc(Br)cc1Cl]

To a solution of 4-bromo-2-chloro-6-fluoro-benzonitrile (400 mg, 1.71 mmol, 1.00 eq) in isopropanol (1.00 mL) was added NH₃·H₂O (4.00 mL). The reaction mixture was stirred at 80° C. for 12 h. A second reaction mixture was set up in parallel, and the two reaction mixtures were combined. The combined mixture was concentrated under reduced pressure to give a residue. The residue was triturated with water (20.0 mL). A solid formed that was collected by filtration and dried to give 2-amino-4-bromo-6-chloro-benzonitrile (600 mg, 76%) as a solid. LCMS: (ESI, m/z): 228.9, 230.9 [M+H]⁺. ¹H NMR (400 MHz, DMSO-d₆) δ ppm 6.97 (d, J=2.45 Hz, 2H) 6.69 (br s, 2H).

### Synthesis of Intermediate G145

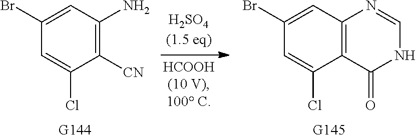 [SMILES: N#Cc1c(N)cc(Br)cc1Cl ; O=c1[nH]cnc2cc(Br)cc(Cl)c12]

To a solution of 2-amino-4-bromo-6-chloro-benzonitrile (600 mg, 2.59 mmol, 1.00 eq) in HCOOH (6.00 mL) was added H₂SO₄ (381 mg, 3.89 mmol, 207 μL, 1.50 eq). The reaction mixture was stirred at 100° C. for 2 h, then poured into ice water (30.0 mL) and stirred for 15 mins. A precipitate formed that was collected by filtration. The resulting solid was washed with petroleum ether (10.0 mL) and dried to give 7-bromo-5-chloro-3H-quinazolin-4-one (450 mg, 67%) as a solid. LCMS: (ESI, m/z): 258.9, 260.9 [M+H]⁺. ¹H NMR (400 MHz, DMSO-d₆) δ ppm 12.36-12.49 (m, 1H) 8.12 (d, J=3.18 Hz, 1H) 7.82 (d, J=1.83 Hz, 1H) 7.77 (d, J=1.96 Hz, 1H).

### Synthesis of Intermediate G146

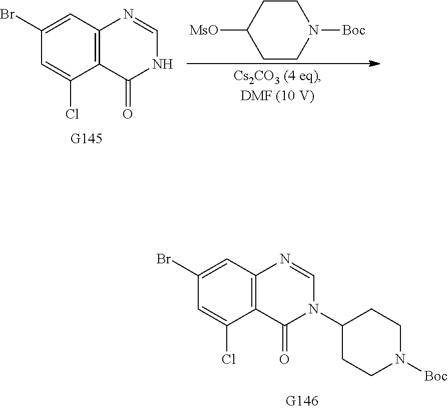 [SMILES: O=c1[nH]cnc2cc(Br)cc(Cl)c12 ; CC(C)(C)OC(=O)N1CCC(OS(C)(=O)=O)CC1 ; CC(C)(C)OC(=O)N1CCC(n2cnc3cc(Br)cc(Cl)c3c2=O)CC1]

To a solution of 7-bromo-5-chloro-3H-quinazolin-4-one (420 mg, 1.62 mmol, 1.00 eq) and tert-butyl 4-methylsulfonyloxypiperidine-1-carboxylate (542 mg, 1.94 mmol, 1.20 eq) in N,N-dimethyl formamide (4.20 mL) was added Cs₂CO₃ (2.11 g, 6.47 mmol, 4.00 eq) at 20° C. The reaction mixture was stirred at 90° C. for 12 h, then cooled to room temperature, poured into water (30.0 mL), and extracted with ethyl acetate (5×10.0 mL). The organic layers were combined, washed with brine (50.0 mL), dried over anhydrous Na₂SO₄, filtered, and the filtrate concentrated under reduced pressure to give a residue. The residue was purified by silica gel column chromatography, eluted with 10%-30% of ethyl acetate in petroleum ether to give a residue, which was further purified by prep-TLC (petroleum ether/ethyl acetate=2/1, Rf=0.6) to give tert-butyl 4-(7-bromo-5-chloro-4-oxo-quinazolin-3-yl) piperidine-1-carboxylate (200 mg, 28%) as a solid. LCMS: (ESI, m/z): 444.0 [M+H]⁺. ¹H NMR (400 MHz, DMSO-d₆) δ ppm 8.52 (s, 1H) 7.82 (dd, J=14.06, 1.96 Hz, 2H) 4.67-4.79 (m, 1H) 4.12 (br dd, J=8.74, 2.02 Hz, 2H) 2.80-2.97 (m, 2H) 1.80-1.96 (m, 4H) 1.42 (s, 9H).

### Synthesis of Intermediate G147

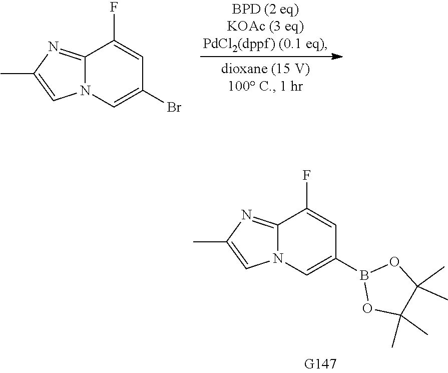

To a mixture of 6-bromo-8-fluoro-2-methyl-imidazo[1,2-a]pyridine (300 mg, 1.31 mmol, 1.00 eq) and 4,4,4′,4′,5,5,5′,5′-octamethyl-2,2′-bi(1,3,2-dioxaborolane) (665 mg, 2.62 mmol, 2.00 eq) in dioxane (4.50 mL) was added potassium acetate (386 mg, 3.93 mmol, 3.00 eq) and cyclopentyl(diphenyl)phosphane; dichloropalladium; iron (95.9 mg, 131 umol, 0.10 eq) under N₂ atmosphere. The reaction mixture was stirred at 100° C. for 1 h, then filtered. The filtrate was concentrated to give 8-fluoro-2-methyl-6-(4,4,5,5-tetramethyl-1,3,2-dioxaborolan-2-yl)imidazo[1,2-a]pyridine (200 mg, 39%) as a solid. LCMS: (ES, m/z): 195.1 [M−82+H]⁺.

### Synthesis of Intermediate G148

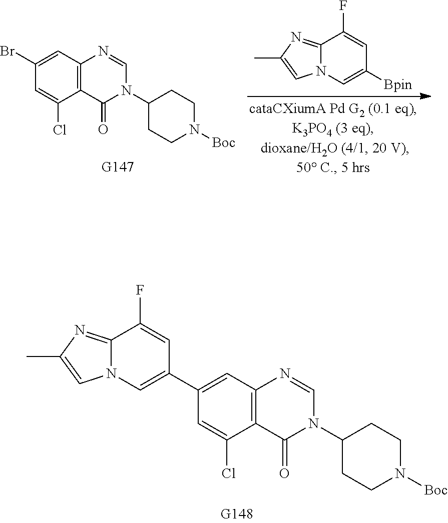

To a mixture of tert-butyl 4-(7-bromo-5-chloro-4-oxo-quinazolin-3-yl)piperidine-1-carboxylate (50.0 mg, 113 umol, 1.00 eq) and 8-fluoro-2-methyl-6-(4,4,5,5-tetramethyl-1,3,2-dioxaborolan-2-yl)imidazo[1,2-a]pyridine (66.8 mg, 169 umol, 70% purity, 1.50 eq) in a mixture of 1,4-dioxane (800 uL) and water (200 uL) was added K₃PO₄ (71.9 mg, 339 umol, 3.00 eq) and [2-(2-aminophenyl)phenyl]-chloro-palladium; bis(1-adamantyl)-butyl-phosphane (7.55 mg, 11.3 umol, 0.10 eq) under N₂ atmosphere. The reaction mixture was stirred at 50° C. for 5 h. Two additional reaction mixtures were set up in parallel and the three reaction mixtures were combined. The combined mixture was filtered and the filter cake was washed with dichloromethane (4×10.0 mL). The organic layers were combined and concentrated to give a residue. The residue was purified by prep-HPLC (Condition G8, Gradient 3) to give tert-butyl 4-[5-chloro-7-(8-fluoro-2-methyl-imidazo[1,2-a]pyridin-6-yl)-4-oxo-quinazolin-3-yl]piperidine-1-carboxylate (40.0 mg, 22%) as a solid. LCMS: (ESI, m/z): 512.2 [M+H]⁺.

### Synthesis of Compound 610

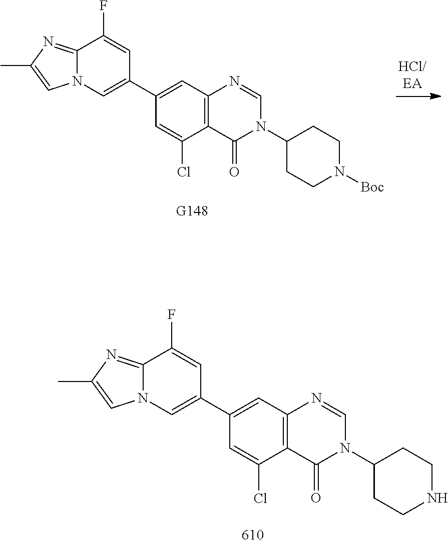 [SMILES: Cc1cn2cc(-c3cc(Cl)c4c(=O)n(C5CCN(C(=O)OC(C)(C)C)CC5)cnc4c3)cc(F)c2n1 ; Cc1cn2cc(-c3cc(Cl)c4c(=O)n(C5CCNCC5)cnc4c3)cc(F)c2n1]

A solution of tert-butyl 4-[5-chloro-7-(8-fluoro-2-methyl-imidazo[1,2-a]pyridin-6-yl)-4-oxo-quinazolin-3-yl]piperidine-1-carboxylate (35.0 mg, 68.36 umol, 1.00 eq) in HCl (gas)/ethyl acetate (4.0 M, 2.00 mL) was stirred at 20° C. for 1 h. The reaction mixture was concentrated to give a solid. The solid was triturated with dichloromethane (3.00 mL), filtered to collect the solid, and the filter cake dried to give 5-chloro-7-(8-fluoro-2-methyl-imidazo[1,2-a]pyridin-6-yl)-3-(4-piperidyl)quinazolin-4-one (32.1 mg, 92%) as a solid. LCMS: (ES, m/z): 412.1 [M+H]⁺. ¹H NMR (400 MHz, METHANOL-d₄) δ ppm 9.10 (s, 1H) 8.34 (s, 1H) 8.22 (d, J=11.44 Hz, 1H) 8.10 (s, 1H) 7.99 (s, 2H) 4.80 (br d, J=1.43 Hz, 1H) 3.58-3.68 (m, 2H) 3.21-3.27 (m, 2H) 2.60 (d, J=0.72 Hz, 3H) 2.45-2.57 (m, 2H) 2.24 (br d, J=12.64 Hz, 2H). ¹⁹F NMR (400 MHz, METHANOL-d₄) δ ppm−133.443 (s, 1F).

### Example G85: Synthesis of Compounds 1437 and 1438

### Synthesis of Intermediate G149

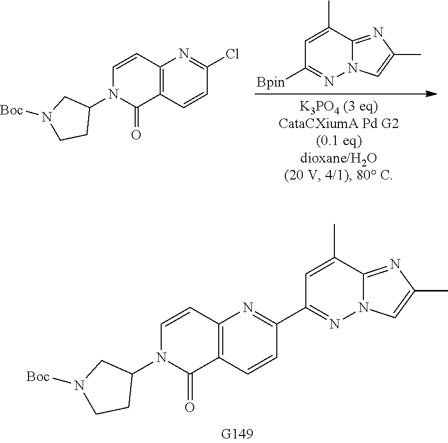

To a mixture of tert-butyl 3-(2-chloro-5-oxo-1,6-naphthyridin-6-yl)pyrrolidine-1-carboxylate (500 mg, 1.43 mmol, 1.00 eq) and 2,8-dimethyl-6-(4,4,5,5-tetramethyl-1,3,2-dioxaborolan-2-yl)imidazo[1,2-b]pyridazine (508 mg, 1.86 mmol, 1.30 eq) in a mixture of 1,4-dioxane (8.00 mL) and water (2.00 mL) was added K₃PO₄ (910 mg, 4.29 mmol, 3.00 eq) and [2-(2-aminophenyl)phenyl]-chloro-palladium; bis(1-adamantyl)-butyl-phosphane (95.6 mg, 143 umol, 0.10 eq) under N₂ atmosphere. The reaction mixture was stirred at 80° C. for 1 h, then cooled to 20° C., filtered, and the filter cake washed with dichloromethane (5×10.0 mL). The filtrate was dried over anhydrous sodium sulfate, filtered, and the filtrate concentrated under reduced pressure to give a residue. The residue was purified by silica gel column chromatography, eluted with 20%-100% of ethyl acetate in petroleum ether, followed by purified by prep-HPLC (Condition G8, Gradient 3) to give tert-butyl 3-[2-(2,8-dimethylimidazo[1,2-b]pyridazin-6-yl)-5-oxo-1,6-naphthyridin-6-yl]pyrrolidine-1-carboxylate (120 mg, 17%) as a solid. LCMS: (ESI, m/z): 461.3 [M+H]⁺. ¹H NMR (400 MHz, DMSO-d₆) δ ppm 8.72 (br d, J=8.31 Hz, 1H) 8.38 (br d, J=8.19 Hz, 1H) 8.16 (br s, 1H) 8.07 (br s, 1H) 7.77 (br d, J=7.09 Hz, 1H) 6.87 (br d, J=7.58 Hz, 1H) 5.25-5.47 (m, 1H) 4.04-4.15 (m, 1H) 3.72-3.79 (m, 1H) 3.49-3.58 (m, 2H) 2.66 (br s, 3H) 2.43 (br s, 3H) 2.23-2.35 (m, 2H) 1.43 (br s, 9H).

### Synthesis of Intermediate G150 and G151

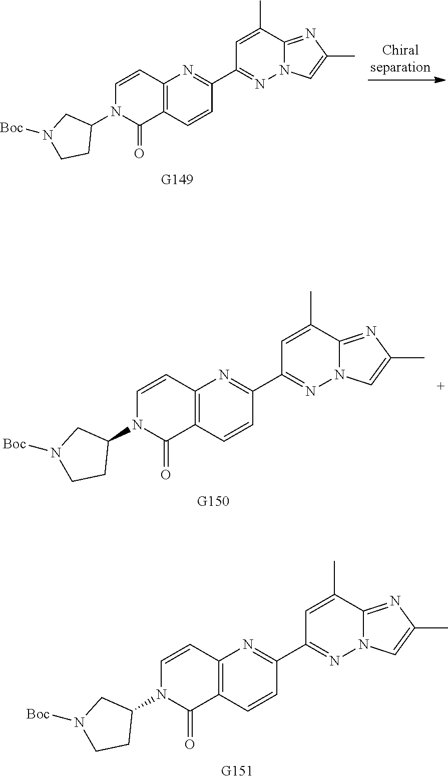 [SMILES: Cc1cn2nc(-c3ccc4c(=O)n(C5CCN(C(=O)OC(C)(C)C)C5)ccc4n3)cc(C)c2n1 ; Cc1cn2nc(-c3ccc4c(=O)n([C@H]5CCN(C(=O)OC(C)(C)C)C5)ccc4n3)cc(C)c2n1 ; Cc1cn2nc(-c3ccc4c(=O)n([C@@H]5CCN(C(=O)OC(C)(C)C)C5)ccc4n3)cc(C)c2n1]

Tert-butyl 3-[2-(2,8-dimethylimidazo[1,2-b]pyridazin-6-yl)-5-oxo-1,6-naphthyridin-6-yl]pyrrolidine-1-carboxylate (120 mg) was purified by SFC to give tert-butyl (3S)-3-[2-(2,8-dimethylimidazo[1,2-b]pyridazin-6-yl)-5-oxo-1,6-naphthyridin-6-yl]pyrolidine-1-carboxylate (32.0 mg, 27%) and tert-butyl (3R)-3-[2-(2,8-dimethylimidazo[1,2-b]pyridazin-6-yl)-5-oxo-1,6-naphthyridin-6-yl]pyrrolidine-1-carboxylate (42.0 mg, 35.0%) as solids. G150: ¹H NMR (400 MHz, DMSO-d₆) δ ppm 8.72 (d, J=8.44 Hz, 1H) 8.38 (d, J=8.44 Hz, 1H) 8.16 (s, 1H) 8.07 (s, 1H) 7.70-7.85 (m, 1H) 6.88 (d, J=7.82 Hz, 1H) 5.30-5.47 (m, 1H) 3.71-3.78 (m, 1H) 3.51-3.56 (m, 1H) 3.40-3.44 (m, 2H) 2.66 (s, 3H) 2.44 (s, 3H) 2.20-2.33 (m, 2H) 1.42 (br s, 9H). G151: ¹H NMR (400 MHz, DMSO-d₆) δ ppm 8.71 (d, J=8.44 Hz, 1H) 8.37 (d, J=8.44 Hz, 1H) 8.15 (s, 1H) 8.06 (s, 1H) 7.77 (br s, 1H) 6.87 (d, J=7.82 Hz, 1H) 5.32-5.44 (m, 1H) 3.70-3.80 (m, 1H) 3.49-3.57 (m, 1H) 3.38-3.45 (m, 2H) 2.66 (s, 3H) 2.43 (s, 3H) 2.20-2.32 (m, 2H) 1.42 (br s, 9H).

### Synthesis of Compound 1437

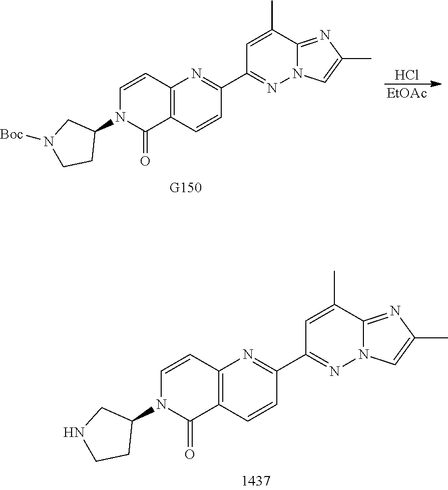 [SMILES: "EtOAc" ; Cc1cn2nc(-c3ccc4c(=O)n([C@H]5CCN(C(=O)OC(C)(C)C)C5)ccc4n3)cc(C)c2n1 ; Cl ; Cc1cn2nc(-c3ccc4c(=O)n([C@H]5CCNC5)ccc4n3)cc(C)c2n1]

A solution of tert-butyl (3S)-3-[2-(2,8-dimethylimidazo[1,2-b]pyridazin-6-yl)-5-oxo-1,6-naphthyridin-6-yl]pyrrolidine-1-carboxylate (28.0 mg, 60.8 umol, 1.00 eq) in HCl (gas)/ethyl acetate (4.0 M, 1.00 mL) was stirred at 20° C. for 2 h. The reaction mixture was concentrated to give a residue. The residue was triturated with dichloromethane (3.00 mL), filtered to collect the solid, and the filter cake dried to give 2-(2,8-dimethylimidazo[1,2-b]pyridazin-6-yl)-6-[(3S)-pyrrolidin-3-yl]-1,6-aphthyridin-5-one (22.8 mg, 83%) as a solid. LCMS: (ESI, m/z): 361.1 [M+H]⁺, ¹H NMR (400 MHz, METHANOL-d₄) δ ppm 8.88 (d, J=8.44 Hz, 1H) 8.77 (s, 1H) 8.56 (d, J=8.44 Hz, 1H) 8.39 (s, 1H) 7.73 (d, J=7.70 Hz, 1H) 6.99 (d, J=7.70 Hz, 1H) 5.08-5.15 (m, 1H) 3.82-3.92 (m, 2H) 3.65-3.72 (m, 1H) 3.37-3.42 (m, 1H) 2.83 (s, 3H) 2.62-2.75 (m, 4H) 2.51 (td, J=9.32, 4.34 Hz, 1H).

### Synthesis of Compound 1438

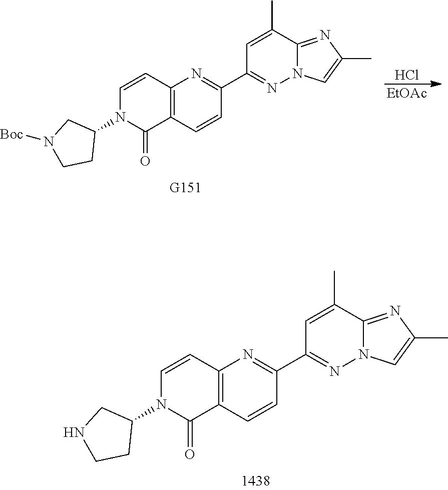 [SMILES: "EtOAc" ; Cc1cn2nc(-c3ccc4c(=O)n([C@@H]5CCN(C(=O)OC(C)(C)C)C5)ccc4n3)cc(C)c2n1 ; Cl ; Cc1cn2nc(-c3ccc4c(=O)n([C@@H]5CCNC5)ccc4n3)cc(C)c2n1]

A solution of tert-butyl (3R)-3-[2-(2,8-dimethylimidazo[1,2-b]pyridazin-6-yl)-5-oxo-1,6-naphthyridin-6-yl]pyrrolidine-1-carboxylate (37.0 mg, 80.3 umol, 1.00 eq) in HCl (gas)/ethyl acetate (4.0 M, 1.00 mL) was stirred at 20° C. for 2 h. The reaction mixture was concentrated to give a solid. The solid lyophilized to afford 2-(2,8-dimethylimidazo[1,2-b]pyridazin-6-yl)-6-[(3R)-pyrrolidin-3-yl]-1,6-naphthyridin-5-one (32.7 mg, 91%) as a solid. LCMS: (ESI, m/z): 361.1 [M+H]⁺. ¹H NMR (400 MHz, METHANOL-d₄) δ ppm 8.88 (d, J=8.44 Hz, 1H) 8.80 (d, J=1.10 Hz, 1H) 8.57 (d, J=8.56 Hz, 1H) 8.41 (s, 1H) 7.73 (d, J=7.82 Hz, 1H) 6.99 (d, J=7.46 Hz, 1H) 5.07-5.15 (m, 1H) 3.82-3.93 (m, 2H) 3.65-3.72 (m, 1H) 3.38 (br d, J=11.13 Hz, 1H) 2.84 (d, J=0.73 Hz, 3H) 2.65-2.76 (m, 4H) 2.45-2.56 (m, 1H).

### Example G86: Synthesis of Compounds 647 and 648

### Synthesis of Intermediate G152A and G152B

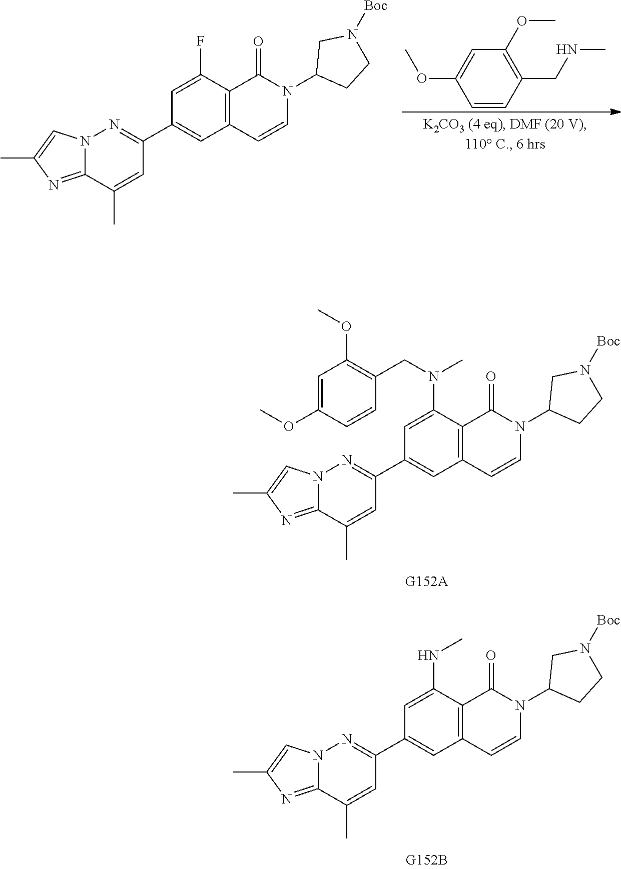

To a solution of tert-butyl 3-[6-(2,8-dimethylimidazo[1,2-b]pyridazin-6-yl)-8-fluoro-1-oxo-2-isoquinolyl]pyrrolidine-1-carboxylate (50.0 mg, 105 umol, 1.00 eq) in N,N-dimethyl formamide (100 uL) was added K₂CO₃ (57.9 mg, 419 umol, 4.00 eq) and 1-(2,4-dimethoxyphenyl)-N-methyl-methanamine (75.9 mg, 419 umol, 4.00 eq). The reaction mixture was stirred at 110° C. for 6 h, then cooled to 20° C. and quenched with ice-water (20.0 mL). The resulting mixture was filtered and the filter cake was dried to give a mixture of tert-butyl 3-[8-[(2,4-dimethoxyphenyl)methyl-methyl-amino]-6-(2,8-dimethylimidazo[1,2-b]pyridazin-6-yl)-1-oxo-2-isoquinolyl]pyrrolidine-1-carboxylate and tert-butyl 3-(6-(2,8-dimethylimidazo[1,2-b]pyridazin-6-yl)-8-(methylamino)-1-oxoisoquinolin-2 (1H)-yl)pyrrolidine-1-carboxylate (310 mg, 77%) as a solid. LCMS: (ESI, m/z): 639.4 [M+H]⁺, 489.3 [M+H]⁺.

### Synthesis of Intermediate G153

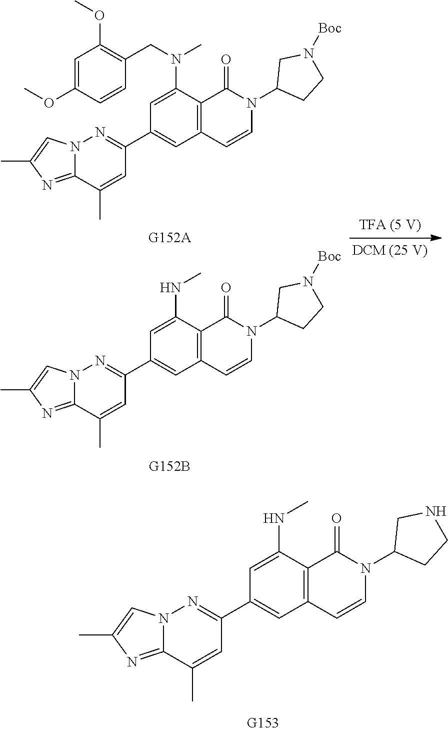 [SMILES: COc1ccc(CN(C)c2cc(-c3cc(C)c4nc(C)cn4n3)cc3ccn(C4CCN(C(=O)OC(C)(C)C)C4)c(=O)c23)c(OC)c1 ; CNc1cc(-c2cc(C)c3nc(C)cn3n2)cc2ccn(C3CCN(C(=O)OC(C)(C)C)C3)c(=O)c12 ; CNc1cc(-c2cc(C)c3nc(C)cn3n2)cc2ccn(C3CCNC3)c(=O)c12]

To a mixture of tert-butyl 3-[8-[(2,4-dimethoxyphenyl)methyl-methyl-amino]-6-(2,8-dimethylimidazo[1,2-b]pyridazin-6-yl)-1-oxo-2-isoquinolyl]pyrrolidine-1-carboxylate and tert-butyl 3-(6-(2,8-dimethylimidazo[1,2-b]pyridazin-6-yl)-8-(methylamino)-1-oxoisoquinolin-2(1H)-yl)pyrrolidine-1-carboxylate (300 mg, 470 umol, 1.00 eq) in dichloromethane (7.50 mL) was added trifluoroacetic acid (1.50 mL). The reaction mixture was stirred at 20° C. for 2 h, then concentrated to give a residue which was purified by prep-HPLC (Condition G8, Gradient 1) to give 6-(2,8-dimethylimidazo[1,2-b]pyridazin-6-yl)-8-(methylamino)-2-pyrrolidin-3-yl-isoquinolin-1-one (100 mg, 55%) as a solid. LCMS: (ESI, m/z): 389.2 [M+H]⁺. ¹H NMR (400 MHz, METHANOL-d₄) δ ppm 8.31 (dd, J=19.07, 1.10 Hz, 2H) 7.41 (d, J=1.59 Hz, 1H) 7.32 (d, J=7.46 Hz, 1H) 7.22 (d, J=1.47 Hz, 1H) 6.66 (d, J=7.46 Hz, 1H) 4.99 (tdd, J=9.15, 9.15, 5.72, 3.00 Hz, 1H) 3.88 (ddd, J=11.58, 8.65, 3.24 Hz, 1H) 3.80 (dd, J=12.90, 2.87 Hz, 1H) 3.63 (dd, J=12.72, 9.17 Hz, 1H) 3.32-3.39 (m, 1H) 3.05 (s, 3H) 2.80 (d, J=0.98 Hz, 3H) 2.66 (d, J=0.86 Hz, 4H) 2.41-2.53 (m, 1H).

### Synthesis of Compound 647 and Compound 648

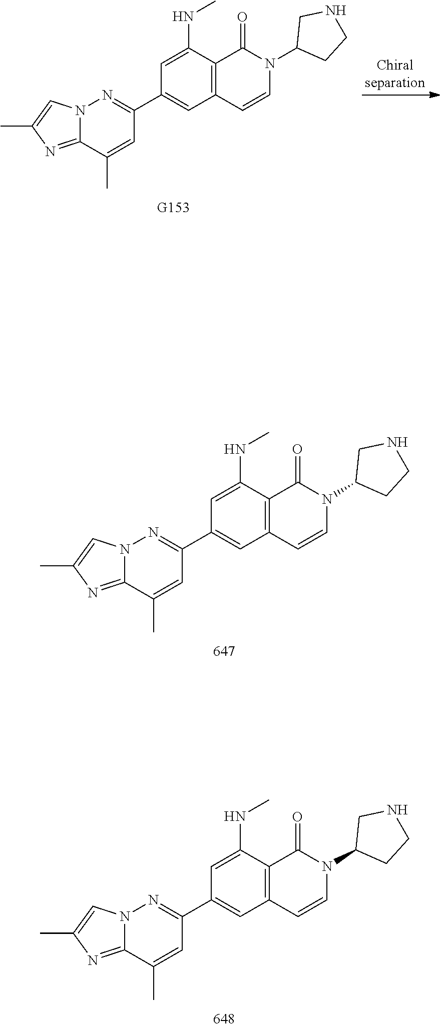 [SMILES: CNc1cc(-c2cc(C)c3nc(C)cn3n2)cc2ccn(C3CCNC3)c(=O)c12 ; CNc1cc(-c2cc(C)c3nc(C)cn3n2)cc2ccn([C@H]3CCNC3)c(=O)c12 ; CNc1cc(-c2cc(C)c3nc(C)cn3n2)cc2ccn([C@@H]3CCNC3)c(=O)c12]

6-(2,8-dimethylimidazo[1,2-b]pyridazin-6-yl)-8-(methylamino)-2-pyrrolidin-3-yl-isoquinolin-1-one (100 mg) was purified by SFC (Condition G3) to give 6-(2,8-dimethylimidazo[1,2-b]pyridazin-6-yl)-8-(methylamino)-2-[(3S)-pyrrolidin-3-yl]isoquinolin-1-one (39.8 mg, 44%) and 6-(2,8-dimethylimidazo[1,2-b]pyridazin-6-yl)-8-(methylamino)-2-[(3R)-pyrrolidin-3-yl]isoquinolin-1-one (19.3 mg, 21%) as solids. 647: LCMS: (ESI, m/z): 389.2 [M+H]⁺. ¹H NMR (400 MHz, METHANOL-d₄) δ ppm 7.88 (s, 1H) 7.53 (s, 1H) 7.28 (d, J=7.46 Hz, 1H) 7.18 (d, J=0.98 Hz, 1H) 7.05 (s, 1H) 6.57 (d, J=7.58 Hz, 1H) 5.17-5.33 (m, 1H) 3.27 (br s, 2H) 2.98-3.11 (m, 5H) 2.64 (s, 3H) 2.48 (s, 3H) 2.29-2.39 (m, 1H) 2.00-2.10 (m, 1H). 648: LCMS: (ESI, m/z): 389.2 [M+H]⁺. ¹H NMR (400 MHz, METHANOL-d₄) δ ppm 7.88 (s, 1H) 7.52 (s, 1H) 7.27 (d, J=7.46 Hz, 1H) 7.18 (d, J=1.10 Hz, 1H) 7.05 (s, 1H) 6.56 (d, J=7.46 Hz, 1H) 5.17-5.29 (m, 1H) 3.27-3.30 (m, 2H) 2.98-3.13 (m, 5H) 2.64 (s, 3H) 2.48 (s, 3H) 2.29-2.40 (m, 1H) 2.00-2.12 (m, 1H).

### Example G87: Synthesis of Compounds 649 and 650

### Synthesis of Intermediate G154

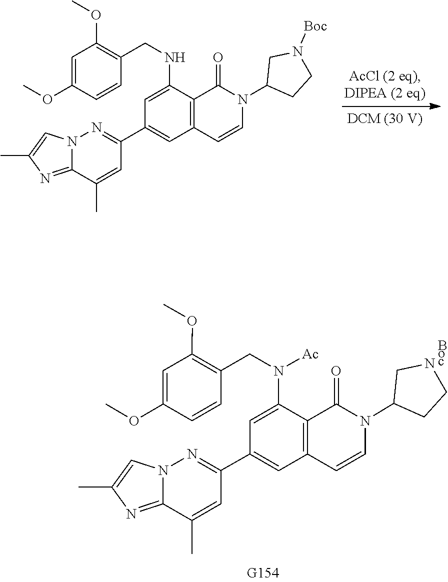 [SMILES: COc1ccc(CNc2cc(-c3cc(C)c4nc(C)cn4n3)cc3ccn(C4CCN(C(=O)OC(C)(C)C)C4)c(=O)c23)c(OC)c1 ; COc1ccc(CN(C(C)=O)c2cc(-c3cc(C)c4nc(C)cn4n3)cc3ccn(C4CCNC4)c(=O)c23)c(OC)c1 ; "Boc"]

To a solution of tert-butyl 3-[8-[(2,4-dimethoxyphenyl)methylamino]-6-(2,8-dimethylimidazo[1,2-b]pyridazin-6-yl)-1-oxo-2-isoquinolyl]pyrrolidine-1-carboxylate (250 mg, 400 umol, 1.00 eq) in CH₂Cl₂ (7.50 mL) was added diisopropylethylamine (103 mg, 800 umol, 139 uL, 2.00 eq) and acetyl chloride (62.8 mg, 800 umol, 57.1 uL, 2.00 eq) at 0° C. The reaction mixture was stirred at 20° C. for 1 h, then poured into water (20.0 mL). The resulting mixture was extracted with dichloromethane (4×10.0 mL). The organic layers were combined, dried over anhydrous Na₂SO₄, filtered, and the filtrate concentrated in vacuum to give a residue which was purified by silica gel chromatography (20% of ethyl acetate in petroleum ether) to give tert-butyl 3-[8-[acetyl-[(2,4-dimethoxyphenyl)methyl]amino]-6-(2,8-dimethylimidazo[1,2-b]pyridazin-6-yl)-1-oxo-2-isoquinolyl]pyrrolidine-1-carboxylate (44-1, 300 mg, 86%) as a solid. LCMS: (ESI, m/z): 667.4 [M+H]⁺. ¹H NMR (400 MHz, DMSO-d₆) δ ppm 8.28 (s, 1H) 8.07 (s, 1H) 7.55 (br d, J=7.82 Hz, 3H) 7.15 (dd, J=8.31, 2.81 Hz, 1H) 6.80 (d, J=7.58 Hz, 1H) 6.40 (ddd, J=8.31, 3.97, 2.38 Hz, 1H) 6.30-6.35 (m, 1H) 5.29-5.46 (m, 1H) 5.18 (dd, J=14.37, 5.69 Hz, 1H) 4.15 (dd, J=14.24, 10.70 Hz, 1H) 3.73 (br d, J=4.28 Hz, 1H) 3.67 (d, J=7.09 Hz, 1H) 3.65 (s, 2H) 3.39 (br d, J=2.57 Hz, 6H) 2.58-2.61 (m, 3H) 2.41 (s, 3H) 2.21 (s, 3H) 1.91 (s, 2H) 1.43 (br s, 9H).

### Synthesis of Intermediate G155

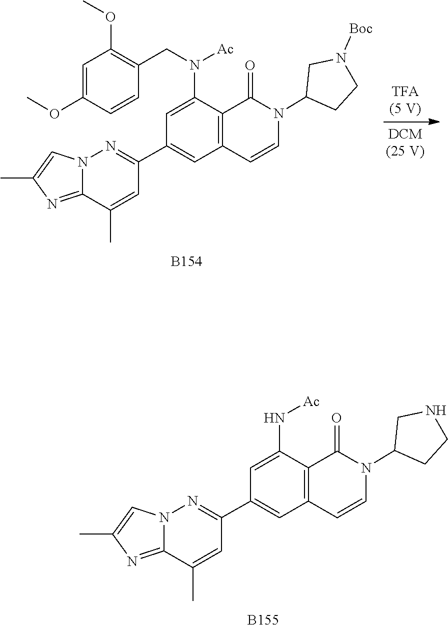 [SMILES: COc1ccc(CN(C(C)=O)c2cc(-c3cc(C)c4nc(C)cn4n3)cc3ccn(C4CCN(C(=O)OC(C)(C)C)C4)c(=O)c23)c(OC)c1 ; "B155" ; CC(=O)Nc1cc(-c2cc(C)c3nc(C)cn3n2)cc2ccn(C3CCNC3)c(=O)c12]

To a solution of tert-butyl 3-[8-[acetyl-[(2,4-dimethoxyphenyl)methyl]amino]-6-(2,8-dimethylimidazo[1,2-b]pyridazin-6-yl)-1-oxo-2-isoquinolyl]pyrrolidine-1-carboxylate (280 mg, 420 umol, 1.00 eq) in dichloromethane (7.00 mL) was added trifluoroacetic acid (1.40 mL). The reaction mixture was stirred at 20° C. for 2 h, then concentrated to give a residue which was purified by prep-HPLC (Condition 8, Gradient 1) to give N-[6-(2,8-dimethylimidazo[1,2-b]pyridazin-6-yl)-1-oxo-2-pyrrolidin-3-yl-8-isoquinolyl]acetamide (100 mg, 57%) as a solid. LCMS: (ESI, m/z): 417.2 [M+H]⁺. ¹H NMR (400 MHz, METHANOL-d₄) δ ppm 12.87 (s, 1H) 9.43 (d, J=1.59 Hz, 1H) 8.36 (d, J=0.86 Hz, 1H) 8.27 (d, J=0.98 Hz, 1H) 8.07 (d, J=1.71 Hz, 1H) 7.51 (d, J=7.46 Hz, 1H) 6.89 (d, J=7.34 Hz, 1H) 5.07-5.18 (m, 1H) 3.85-3.95 (m, 2H) 3.70 (dd, J=12.84, 9.17 Hz, 1H) 3.35-3.44 (m, 1H) 2.81 (d, J=0.86 Hz, 3H) 2.67 (d, J=0.86 Hz, 4H) 2.48-2.59 (m, 1H) 2.33 (s, 3H).

### Synthesis of Compound 649 and 650

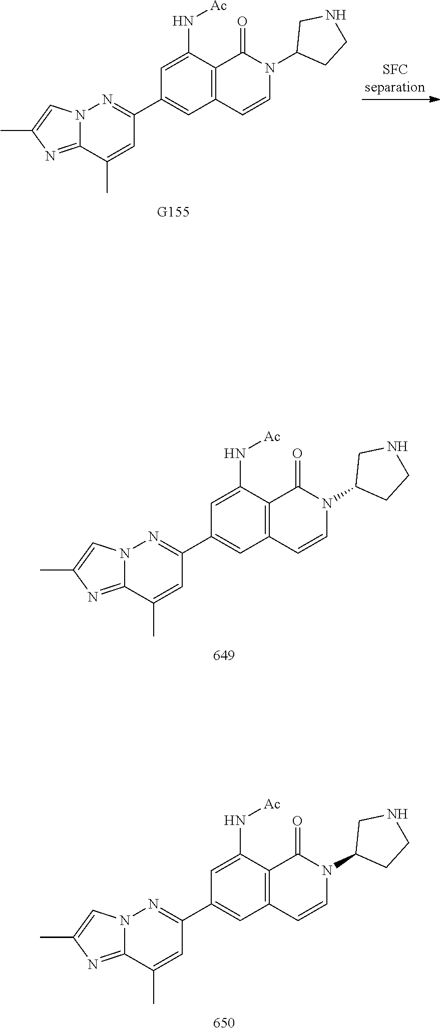 [SMILES: CC(=O)Nc1cc(-c2cc(C)c3nc(C)cn3n2)cc2ccn(C3CCNC3)c(=O)c12 ; CC(=O)Nc1cc(-c2cc(C)c3nc(C)cn3n2)cc2ccn([C@H]3CCNC3)c(=O)c12 ; CC(=O)Nc1cc(-c2cc(C)c3nc(C)cn3n2)cc2ccn([C@@H]3CCNC3)c(=O)c12]

N-[6-(2,8-dimethylimidazo[1,2-b]pyridazin-6-yl)-1-oxo-2-pyrrolidin-3-yl-8-isoquinolyl]acetamide (100 mg) was purified by SFC (Condition G3) to give N-[6-(2,8-dimethylimidazo[1,2-b]pyridazin-6-yl)-1-oxo-2-[(3S)-pyrrolidin-3-yl]-8-isoquinolyl]acetamide (31.6 mg, 32%) and N-[6-(2,8-dimethylimidazo[1,2-b]pyridazin-6-yl)-1-oxo-2-[(3R)-pyrrolidin-3-yl]-8-isoquinolyl]acetamide (54.7 mg, 55%) as solids. 649: LCMS: (ESI, m/z): 417.2 [M+H]⁺. ¹H NMR (400 MHz, METHANOL-d₄) δ ppm 9.04 (d, J=1.71 Hz, 1H) 7.75 (d, J=0.61 Hz, 1H) 7.65 (d, J=1.59 Hz, 1H) 7.41 (d, J=7.46 Hz, 1H) 7.36 (d, J=0.98 Hz, 1H) 6.64 (d, J=7.46 Hz, 1H) 5.25-5.42 (m, 1H) 3.32-3.37 (m, 1H) 3.24-3.28 (m, 1H) 2.98-3.09 (m, 2H) 2.56 (d, J=0.61 Hz, 3H) 2.45 (s, 3H) 2.34-2.40 (m, 1H) 2.25 (s, 3H) 1.99-2.09 (m, 1H). 650: LCMS: (ESI, m/z): 417.2 [M+H]⁺. ¹H NMR (400 MHz, METHANOL-d₄) δ ppm 8.98 (d, J=1.34 Hz, 1H) 7.71 (s, 1H) 7.60 (d, J=1.34 Hz, 1H) 7.38 (br d, J=7.46 Hz, 1H) 7.30 (s, 1H) 6.61 (d, J=7.46 Hz, 1H) 5.31 (br s, 1H) 3.33-3.39 (m, 1H) 3.30 (br s, 1H) 2.99-3.16 (m, 2H) 2.54 (s, 3H) 2.44 (s, 3H) 2.34-2.41 (m, 1H) 2.24 (s, 3H) 2.00-2.12 (m, 1H).

### Example G88: Synthesis of Compound 1469

### Synthesis of Intermediate G156

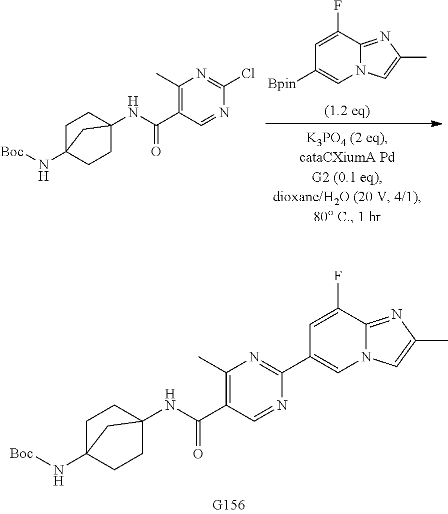

To a solution of tert-butyl N-[4-[(2-chloro-4-methyl-pyrimidine-5-carbonyl)amino]norbornan-1-yl]carbamate (150 mg, 393 umol, 1.00 eq) in 1,4-in dioxane (2.40 mL) and water (0.60 mL) was added 8-fluoro-2-methyl-6-(4,4,5,5-tetramethyl-1,3,2-dioxaborolan-2-yl)imidazo[1,2-a]pyridine (261 mg, 472 umol, 50% purity, 1.20 eq), K₃PO₄ (167 mg, 788 umol, 2.00 eq), and [2-(2-aminophenyl)phenyl]-chloro-palladium; bis(1-adamantyl)-butyl-phosphane (26.3 mg, 39.4 umol, 0.10 eq) at 25° C. under N₂ atmosphere. The reaction mixture was stirred at 80° C. for 1 h, then cooled to 25° C., poured into water (20.0 mL), and extracted with ethyl acetate (3×20.0 mL). The organic layers were combined, washed with brine (50.0 mL), dried over anhydrous Na₂SO₄, filtered, and the filtrate concentrated to give a residue. The residue was purified by silica gel column chromatography (petroleum ether/ethyl acetate=1/1) to give tert-butyl N-[4-[[2-(8-fluoro-2-methyl-imidazo[1,2-a]pyridin-6-yl)-4-methyl-pyrimidine-5-carbonyl]amino]norbornan-1-yl]carbamate (0102-1, 150 mg, 77%) as a solid. LCMS: (ESI, m/z): 495.3 [M+H]⁺. ¹H NMR (400 MHz, DMSO-d₆) δ ppm 9.41 (d, J=1.22 Hz, 1H) 8.75 (s, 1H) 8.70 (s, 1H) 8.03 (d, J=2.32 Hz, 1H) 7.85 (dd, J=12.35, 1.22 Hz, 1H) 7.02 (br d, J=1.22 Hz, 1H) 2.59 (s, 3H) 2.38 (s, 3H) 2.02 (s, 2H) 1.94-1.99 (m, 2H) 1.79-1.90 (m, 4H) 1.68 (br d, J=7.46 Hz, 2H) 1.39 (s, 9H).

### Synthesis of Intermediate G157

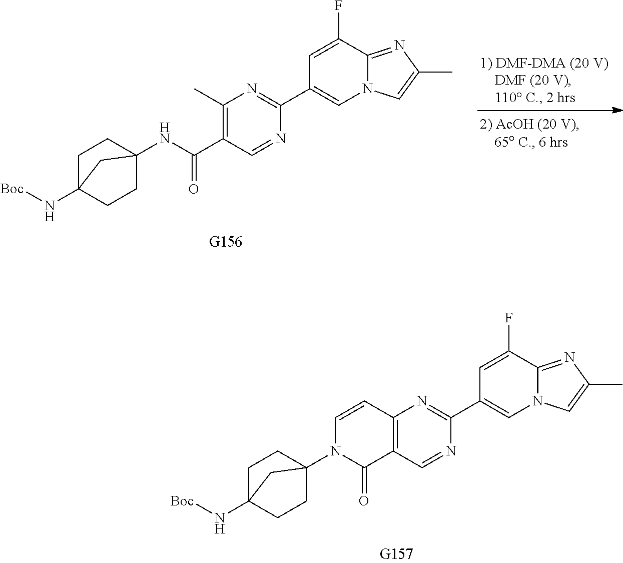 [SMILES: Cc1cn2cc(-c3ncc(C(=O)NC45CCC(NC(=O)OC(C)(C)C)(CC4)C5)c(C)n3)cc(F)c2n1 ; Cc1cn2cc(-c3ncc4c(=O)n(C56CCC(NC(=O)OC(C)(C)C)(CC5)C6)ccc4n3)cc(F)c2n1]

To a solution of tert-butyl N-[4-[[2-(8-fluoro-2-methyl-imidazo[1,2-a]pyridin-6-yl)-4-methyl-pyrimidine-5-carbonyl]amino]norbornan-1-yl]carbamate (150 mg, 303 umol, 1.00 eq) in N,N-dimethyl formamide (3.00 mL) was added dimethyl formamide-N,N-dimethylacetamide (3.00 mL) at 25° C. under N₂ atmosphere. The reaction mixture was stirred at 110° C. for 2 h, then concentrated to give a residue. To the residue was added acetic acid (3.00 mL), and the resulting mixture was stirred at 65° C. for 6 h, then cooled to 25° C. The reaction mixture was concentrated under reduced pressure to give a residue which was triturated with tert-butyl methyl ether (5.00 mL), filtered, and the filter cake dried under reduced pressure to give tert-butyl N-[4-[2-(8-fluoro-2-methyl-imidazo[1,2-a]pyridin-6-yl)-5-oxo-pyrido[4,3-d]pyrimidin-6-yl]norbornan-1-yl]carbamate (100 mg, 73%) as a solid. LCMS: (ESI, m/z): 505.3 [M+H]⁺. ¹H NMR (400 MHz, DMSO-d₆) δ ppm 9.54 (d, J=1.25 Hz, 1H) 9.49 (s, 1H) 8.05 (d, J=2.25 Hz, 1H) 8.00 (d, J=7.88 Hz, 1H) 7.91-7.95 (m, 1H) 7.10-7.20 (m, 1H) 6.63 (d, J=7.88 Hz, 1H) 2.39 (s, 3H) 2.36 (br s, 2H) 2.02-2.08 (m, 2H) 1.80-1.85 (m, 2H) 1.68-1.80 (m, 4H) 1.41 (s, 9H).

### Synthesis of Compound 1469

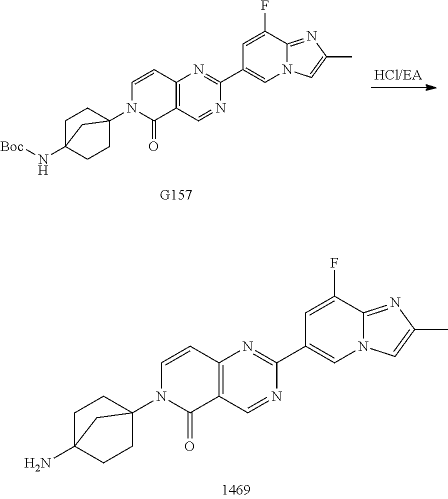 [SMILES: Cc1cn2cc(-c3ncc4c(=O)n(C56CCC(NC(=O)OC(C)(C)C)(CC5)C6)ccc4n3)cc(F)c2n1 ; Cc1cn2cc(-c3ncc4c(=O)n(C56CCC(N)(CC5)C6)ccc4n3)cc(F)c2n1]

To a solution of tert-butyl N-[4-[2-(8-fluoro-2-methyl-imidazo[1,2-a]pyridin-6-yl)-5-oxo-pyrido[4,3-d]pyrimidin-6-yl]norbornan-1-yl]carbamate (100 mg, 198 umol, 1.00 eq) in ethyl acetate (2.00 mL) was added HCl/ethyl acetate (4.0 M, 2.00 mL) at 25° C. The reaction mixture was stirred at 25° C. for 4 h, then concentrated under reduced pressure to give a residue. The residue was triturated with methyl tert-butyl ether (5.00 mL), filtered, and the filter cake concentrated under reduced pressure to give a residue. The residue was purified by prep-HPLC (Condition G8, Gradient 1) to give 6-(4-aminonorbornane-1-yl)-2-(8-fluoro-2-methyl-imidazo[1,2-a]pyridin-6-yl)pyrido[4,3-d]pyrimidin-5-one (35.3 mg, 44%) as a solid. LCMS: (ESI, m/z): 405.2 [M+H]⁺. ¹H NMR (400 MHz, METHANOL-d₄) δ ppm 9.58-9.62 (m, 2H) 8.39 (d, J=11.54 Hz, 1H) 8.02 (d, J=1.76 Hz, 1H) 7.94 (d, J=7.91 Hz, 1H) 6.78 (d, J=8.03 Hz, 1H) 2.68-2.76 (m, 2H) 2.55 (s, 2H) 2.53 (s, 3H) 2.14-2.22 (m, 2H) 2.06-2.13 (m, 2H) 1.99-2.05 (m, 2H) ¹⁹F NMR (376 MHz, METHANOL-d₄) δ ppm−133.978 (s, 1F).

### Example G89: Synthesis of Compound 1480

### Synthesis of Intermediate G158

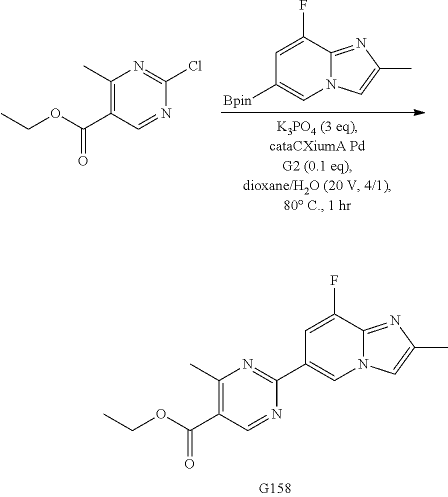

To a mixture of ethyl 2-chloro-4-methyl-pyrimidine-5-carboxylate (500 mg, 2.49 mmol, 1.00 eq) and 8-fluoro-2-methyl-6-(4,4,5,5-tetramethyl-1,3,2-dioxaborolan-2-yl)imidazo[1,2-a]pyridine (826 mg, 2.99 mmol, 1.20 eq) in a mixture of 1,4-dioxane (16.0 mL) and water (4.00 mL) was added tripotassium phosphate (1.59 g, 7.48 mmol, 3.00 eq) and [2-(2-aminophenyl)phenyl]-chloro-palladium; bis(1-adamantyl)-butyl-phosphane (167 mg, 249 umol, 0.10 eq) under N₂ atmosphere. The reaction mixture was stirred at 80° C. for 1 h, then cooled to 20° C. and poured into water (20.0 mL). The resulting mixture was extracted with ethyl acetate (4×10.0 mL). The organic layers were combined, dried over anhydrous Na₂SO₄, filtered, and the filtrate concentrated to give a residue which was purified by silica gel column chromatography (petroleum ether/ethyl acetate=1/50 to 1/1 over 40 min.) to give ethyl 2-(8-fluoro-2-methyl-imidazo[1,2-a]pyridin-6-yl)-4-methyl-pyrimidine-5-carboxylate (620 mg, 57%) as a solid. LCMS: (ESI, m/z): 315.1 [M+H]⁺. ¹H NMR (400 MHz, DMSO-d₆) δ ppm 9.46 (d, J=1.26 Hz, 1H) 9.15 (s, 1H) 8.05 (d, J=2.51 Hz, 1H) 7.87 (dd, J=12.36, 1.19 Hz, 1H) 4.37 (q, J=7.15 Hz, 2H) 2.82 (s, 3H) 2.38 (s, 3H) 1.36 (t, J=7.09 Hz, 3H).

### Synthesis of Intermediate G159

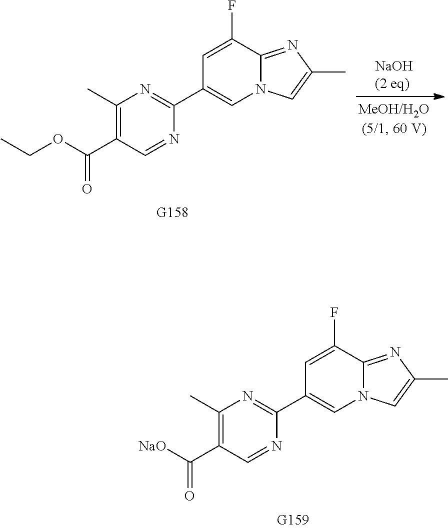

To a solution of ethyl 2-(8-fluoro-2-methyl-imidazo[1,2-a]pyridin-6-yl)-4-methyl-pyrimidine-5-carboxylate (300 mg, 954 umol, 1.00 eq) in a mixture of methanol (15.0 mL) and water (3.00 mL) was added NaOH (114.53 mg, 2.86 mmol, 3.00 eq). The reaction mixture was stirred at 20° C. for 3 h. A second reaction was set up in parallel and the two reaction mixtures were combined. The combined reaction mixture was concentrated under reduced pressure to give a residue. The residue was triturated with dichloromethane (10.0 mL) to give a suspension. The resulting solid was collected by filtration and dried to give [2-(8-fluoro-2-methyl-imidazo[1,2-a]pyridin-6-yl)-4-methyl-pyrimidine-5-carbonyl]oxysodium (400 mg, 68%) as a solid. LCMS: (ESI, m/z): 287.1 [M−23+H]⁺.

### Synthesis of Intermediate G160

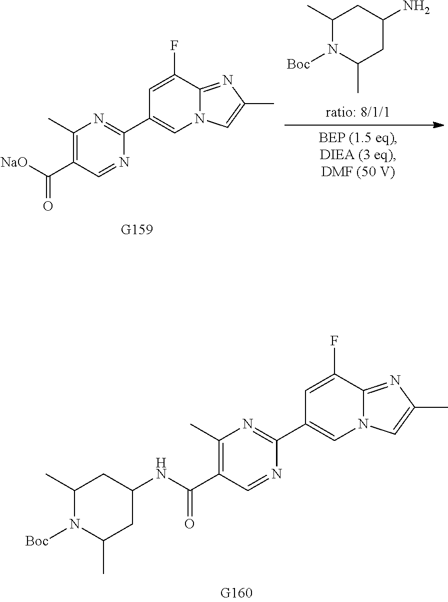

To a solution of [2-(8-fluoro-2-methyl-imidazo[1,2-a]pyridin-6-yl)-4-methyl-pyrimidine-5-carbonyl]oxysodium (250 mg, 811 umol, 1.00 eq) in N,N-dimethyl formamide (12.5 mL) was added 2-bromo-1-ethyl-pyridin-1-ium; tetrafluoroborate (333 mg, 1.22 mmol, 1.50 eq), diisopropylamine (314 mg, 2.43 mmol, 3.00 eq), and tert-butyl 4-amino-2,6-dimethyl-piperidine-1-carboxylate (204 mg, 892 umol, 1.10 eq). The reaction mixture was stirred at 30° C. for 2 h, then poured into water (50.0 mL). A precipitate formed that was collected by filtration and dried to give tert-butyl 4-[[2-(8-fluoro-2-methyl-imidazo[1,2-a]pyridin-6-yl)-4-methyl-pyrimidine-5-carbonyl]amino]-2,6-dimethyl-piperidine-1-carboxylate (0117-3, 340 mg, 56%) as a solid. LCMS: (ESI, m/z): 497.3 [M+H]⁺. ¹H NMR (400 MHz, DMSO-d₆) δ ppm 9.41 (d, J=1.38 Hz, 1H) 8.79 (s, 1H) 8.04 (br s, 1H) 7.85 (dd, J=8.94, 1.19 Hz, 1H) 4.23-4.41 (m, 3H) 2.62-2.63 (m, 3H) 2.38 (s, 3H) 1.81-1.88 (m, 2H) 1.56 (dt, J=12.19, 6.16 Hz, 2H) 1.41-1.42 (m, 9H) 1.23 (d, J=7.00 Hz, 6H). ¹⁹F NMR (376 MHz, DMSO-d₆) δ ppm−132.018 (s, 1F).

### Synthesis of Intermediate G161

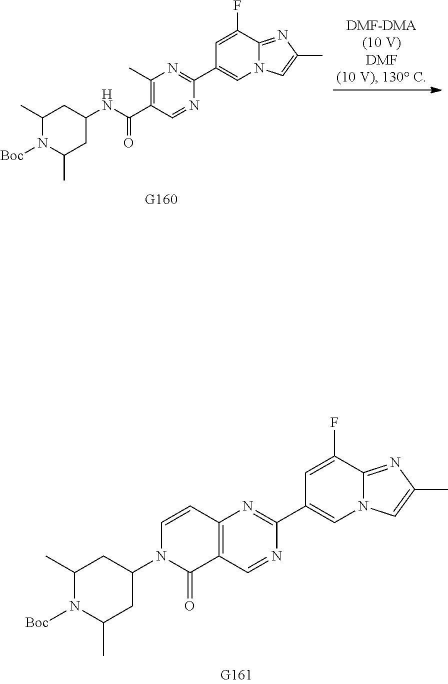

To a solution of tert-butyl 4-[[2-(8-fluoro-2-methyl-imidazo[1,2-a]pyridin-6-yl)-4-methyl-pyrimidine-5-carbonyl]amino]-2,6-dimethyl-piperidine-1-carboxylate (170 mg, 342 umol, 1.00 eq) in N,N-dimethyl formamide (1.70 mL) was added 1,1-dimethoxy-N,N-dimethylmethanamine (1.70 mL). The mixture was stirred at 130° C. for 2 h, then cooled to 20° C. and filtered. The filter cake was washed with methyl tertiary butyl ether (3.00 mL) and dried to give tert-butyl 4-[2-(8-fluoro-2-methyl-imidazo[1,2-a]pyridin-6-yl)-5-oxo-pyrido[4,3-d]pyrimidin-6-yl]-2,6-dimethyl-piperidine-1-carboxylate (170 mg, 49%) as a solid. LCMS: (ESI, m/z): 507.5 [M+H]⁺.

### Synthesis of Intermediate G162 and G163

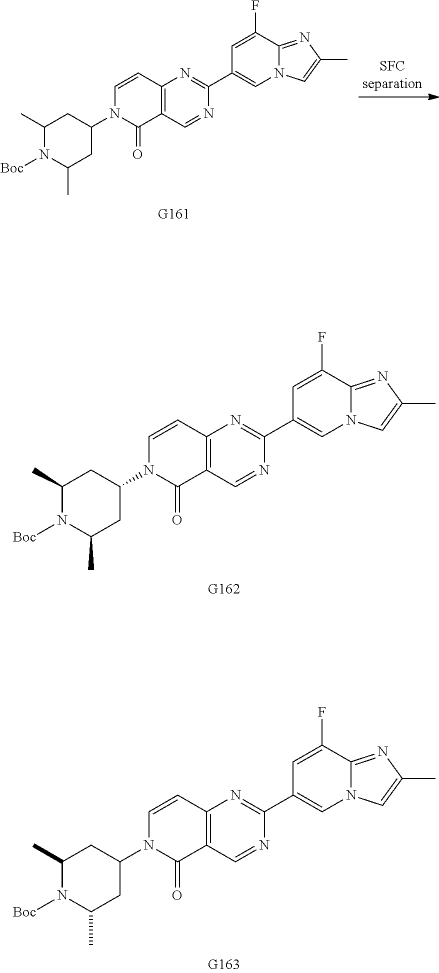

Tert-butyl 4-[2-(8-fluoro-2-methyl-imidazo[1,2-a]pyridin-6-yl)-5-oxo-pyrido[4,3-d]pyrimidin-6-yl]-2,6-dimethyl-piperidine-1-carboxylate (170 mg) was purified by SFC (Condition G3) to give tert-butyl (2R,6S)-4-[2-(8-fluoro-2-methyl-imidazo[1,2-a]pyridin-6-yl)-5-oxo-pyrido[4,3-d]pyrimidin-6-yl]-2,6-dimethyl-piperidine-1-carboxylate (90.0 mg, 53%) and tert-butyl 4-[2-(8-fluoro-2-methyl-imidazo[1,2-a]pyridin-6-yl)-5-oxo-pyrido[4,3-d]pyrimidin-6-yl]-2,6-dimethyl-piperidine-1-carboxylate (35.0 mg, 21%) as solids. G162: LCMS: (ESI, m/z): 507.3 [M+H]⁺. ¹H NMR (400 MHz, DMSO-d₆) δ ppm 9.54 (s, 2H) 8.12 (d, J=7.78 Hz, 1H) 8.06 (d, J=2.64 Hz, 1H) 7.94 (dd, J=12.23, 1.07 Hz, 1H) 6.73 (d, J=7.78 Hz, 1H) 5.34-5.46 (m, 1H) 4.36-4.50 (m, 2H) 2.39 (s, 3H) 2.06-2.14 (m, 2H) 1.74 (br dd, J=12.36, 1.94 Hz, 2H) 1.44 (s, 9H) 1.29 (br d, J=5.90 Hz, 6H). ¹⁹F NMR (376 MHz, DMSO-d₆) δ ppm−131.933 (s, 1F). G163: LCMS: (ESI, m/z): 507.3 [M+H]⁺. ¹H NMR (400 MHz, DMSO-d₆) δ ppm 9.46-9.60 (m, 2H) 8.10 (d, J=7.78 Hz, 1H) 8.06 (d, J=3.14 Hz, 1H) 7.93 (dd, J=12.23, 1.19 Hz, 1H) 6.73 (d, J=7.65 Hz, 1H) 5.12-5.25 (m, 1H) 4.38-4.48 (m, 1H) 3.65-3.74 (m, 1H) 2.39 (s, 3H) 1.93-2.07 (m, 2H) 1.73-1.82 (m, 2H) 1.43-1.46 (m, 9H) 1.28 (d, J=7.03 Hz, 3H). ¹⁹F NMR (376 MHz, DMSO-d₆) δ ppm−131.938 (s, 1F).

### Synthesis of Compound 1480

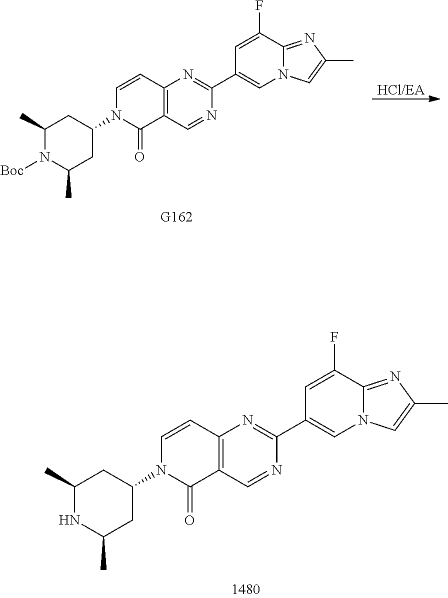

To a solution of tert-butyl (2R,6S)-4-[2-(8-fluoro-2-methyl-imidazo[1,2-a]pyridin-6-yl)-5-oxo-pyrido[4,3-d]pyrimidin-6-yl]-2,6-dimethyl-piperidine-1-carboxylate (80.0 mg, 158 umol, 1.00 eq) in ethyl acetate (3.00 mL) was added HCl (gas)/ethyl acetate (4.0 M, 3.00 mL). The reaction mixture was stirred at 20° C. for 1 h, then concentrated under reduced pressure to give a solid which was triturated with dichloromethane (3.00 mL). The resulting suspension was filtered and the filter cake dried to give 6-[(2R,6S)-2,6-dimethyl-4-piperidyl]-2-(8-fluoro-2-methyl-imidazo[1,2-a]pyridin-6-yl)pyrido[4,3-d]pyrimidin-5-one (74.6 mg, 98%) as a solid. LCMS: (ESI, m/z): 407.2 [M+H]⁺. ¹H NMR (400 MHz, METHANOL-d₄) δ ppm 9.67 (d, J=8.91 Hz, 2H) 8.59 (d, J=11.29 Hz, 1H) 8.00-8.18 (m, 2H) 6.90 (d, J=7.78 Hz, 1H) 5.06 (br s, 1H) 3.64 (ddd, J=12.23, 6.02, 3.33 Hz, 2H) 2.59 (s, 3H) 2.53 (br d, J=16.06 Hz, 2H) 2.03-2.16 (m, 2H) 1.41 (d, J=6.40 Hz, 6H). ¹⁹F NMR (376 MHz, DMSO-d₆) δ ppm−133.698 (s, 1F).

### Example G90: Synthesis of Compounds 1482 and 1483

### Synthesis of Intermediate G164

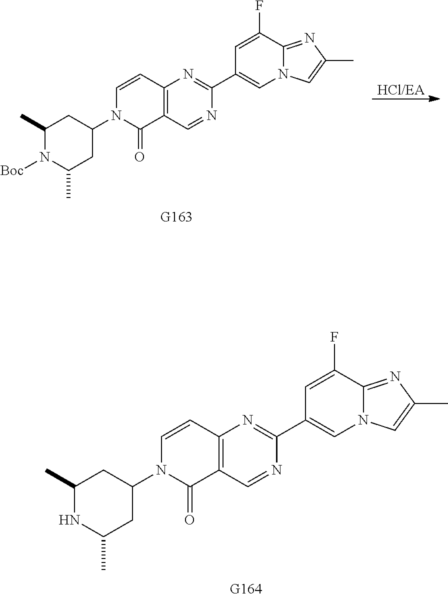

To a solution of tert-butyl 4-[2-(8-fluoro-2-methyl-imidazo[1,2-a]pyridin-6-yl)-5-oxo-pyrido[4,3-d]pyrimidin-6-yl]-2,6-dimethyl-piperidine-1-carboxylate (35.0 mg, 69.1 umol, 1.00 eq) in ethyl acetate (1.00 mL) was added HCl (gas)/ethyl acetate (4.0 M, 2.00 mL). The reaction mixture was stirred at 20° C. for 1 h, then concentrated under reduced pressure to give a solid which was triturated with dichloromethane (2.00 mL). The resulting mixture was filtered and the filter cake dried to give 6-(2,6-dimethyl-4-piperidyl)-2-(8-fluoro-2-methyl-imidazo[1,2-a]pyridin-6-yl)pyrido[4,3-d]pyrimidin-5-one (30.0 mg, 98%) as a solid. LCMS: (ES, m/z): 407.2 [M+H]⁺. 1H NMR (400 MHz, METHANOL-d₄) δ ppm 9.68 (d, J=13.30 Hz, 2H) 8.59 (d, J=11.42 Hz, 1H) 8.14 (s, 1H) 7.95 (d, J=7.91 Hz, 1H) 6.89 (d, J=7.78 Hz, 1H) 5.24-5.39 (m, 1H) 3.98-4.10 (m, 1H) 3.72-3.86 (m, 1H) 2.58 (s, 3H) 2.39 (td, J=13.18, 5.52 Hz, 1H) 2.19-2.27 (m, 1H) 2.03-2.15 (m, 2H) 1.59 (d, J=7.15 Hz, 3H) 1.44 (d, J=6.40 Hz, 3H). ¹⁹F NMR (376 MHz, METHANOL-d₄) δ ppm−134.002 (s, 1F).

### Synthesis of Compound 1482 and 1483

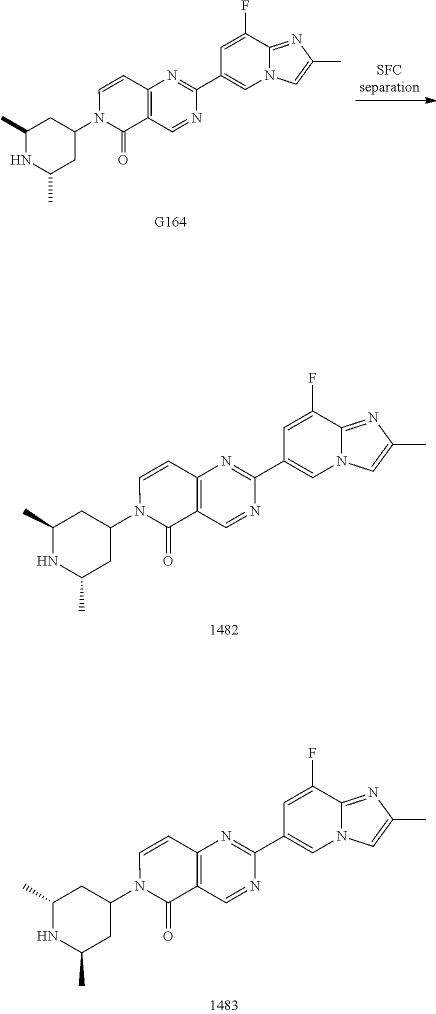

6-(2,6-dimethyl-4-piperidyl)-2-(8-fluoro-2-methyl-imidazo[1,2-a]pyridin-6-yl)pyrido[4,3-d]pyrimidin-5-one (30.0 mg) was purified by SFC (Condition G3) to give 6-[(2S,6S)-2,6-dimethyl-4-piperidyl]-2-(8-fluoro-2-methyl-imidazo[1,2-a]pyridin-6yl)pyrido[4,3-d]pyrimidin-5-one (7.40 mg, 27%) and 6-[(2R,6R)-2,6-dimethyl-4-piperidyl]-2-(8-fluoro-2-methyl-imidazo[1,2-a]pyridin-6-yl)pyrido[4,3-d]pyrimidin-5-one (9.21 mg, 34%) as solids. 1482: LCMS: (ESI, m/z): 407.2 [M+H]⁺. ¹H NMR (400 MHz, METHANOL-d₄) δ ppm 9.59 (s, 1H) 9.45 (s, 1H) 8.08 (d, J=12.13 Hz, 1H) 7.94 (d, J=7.75 Hz, 1H) 7.84 (br d, J=1.00 Hz, 1H) 6.82 (d, J=7.63 Hz, 1H) 5.25-5.40 (m, 1H) 3.51-3.60 (m, 1H) 3.21-3.29 (m, 1H) 2.46 (s, 3H) 1.91-2.06 (m, 2H) 1.80 (br dd, J=10.69, 1.94 Hz, 1H) 1.52 (q, J=12.13 Hz, 1H) 1.40 (d, J=7.00 Hz, 3H) 1.19 (d, J=6.25 Hz, 3H). ¹⁹F NMR (376 MHz, METHANOL-d₄) δ ppm−133.948 (s, 1F). 1483: LCMS: (ESI, m/z): 407.2 [M+H]⁺. ¹H NMR (400 MHz, METHANOL-d₄) δ ppm 9.58 (s, 1H) 9.44 (s, 1H) 8.07 (d, J=11.51 Hz, 1H) 7.94 (d, J=7.63 Hz, 1H) 7.83 (br s, 1H) 6.81 (br d, J=7.38 Hz, 1H) 5.27-5.36 (m, 1H) 3.57 (br d, J=4.38 Hz, 1H) 3.21-3.29 (m, 1H) 2.45 (s, 3H) 1.91-2.05 (m, 2H) 1.76-1.86 (m, 1H) 1.52 (q, J=11.92 Hz, 1H) 1.39 (d, J=6.88 Hz, 3H) 1.18 (d, J=6.25 Hz, 3H). ¹⁹F NMR (376 MHz, METHANOL-d₄) δ ppm−133.952 (s, 1F).

### Example G91: Synthesis of Compound 1507

### Synthesis of Intermediate G165

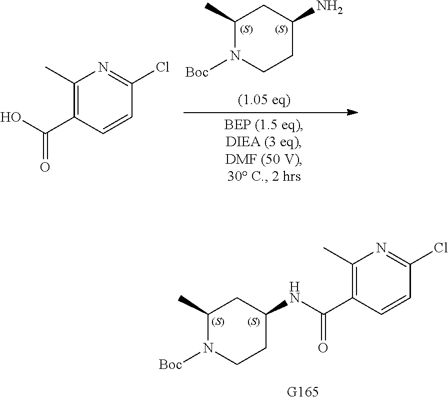

To a mixture of 6-chloro-2-methyl-pyridine-3-carboxylic acid (1.50 g, 8.74 mmol, 1.00 eq) and tert-butyl (2S,4S)-4-amino-2-methyl-piperidine-1-carboxylate (1.97 g, 9.18 mmol, 1.05 eq) in N,N-dimethyl formamide (75.0 mL) was added 2-bromo-1-ethyl-pyridin-1-ium; tetrafluoroborate (3.59 g, 13.1 mmol, 1.50 eq) and N,N-diisopropylethylamine (3.39 g, 26.2 mmol, 4.57 mL, 3.00 eq) at 25° C. under N₂ atmosphere. The reaction mixture was stirred at 30° C. for 2 h, then poured into water (30.0 mL) and extracted with ethyl acetate (3×30.0 mL). The organic layers were combined, washed with brine (30.0 mL), dried over anhydrous Na₂SO₄, filtered, and the filtrate concentrated to give a residue. The residue was purified by silica gel column chromatography (petroleum ether/ethyl acetate=2/1) to give tert-butyl (2S,4S)-4-[(6-chloro-2-methyl-pyridine-3-carbonyl)amino]-2-methyl-piperidine-1-carboxylate (0162-1, 1.50 g, 47%) as an oil. LCMS: (ESI, m/z): 368.3 [M+H]⁺. ¹H NMR (400 MHz, METHANOL-d₄) δ ppm 7.73 (d, J=8.03 Hz, 1H) 7.34 (d, J=8.03 Hz, 1H) 4.08-4.16 (m, 2H) 3.71-3.81 (m, 1H) 3.24-3.30 (m, 1H) 2.55 (s, 3H) 2.02-2.12 (m, 1H) 1.93-2.00 (m, 1H) 1.81 (dt, J=13.90, 7.04 Hz, 1H) 1.59-1.68 (m, 1H) 1.47 (s, 9H) 1.27 (d, J=6.78 Hz, 3H).

### Synthesis of Intermediate G166

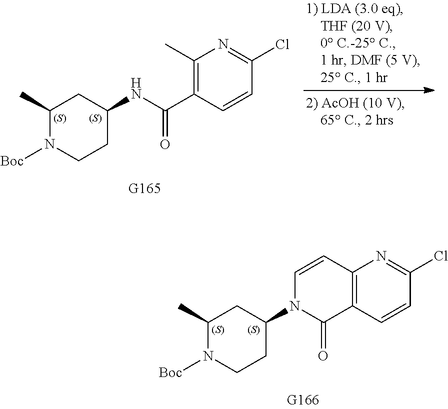

To a solution of tert-butyl (2S,4S)-4-[(6-chloro-2-methyl-pyridine-3-carbonyl)amino]-2-methyl-piperidine-1-carboxylate (2.00 g, 5.44 mmol, 1.00 eq) in tetrahydrofuran (40.0 mL) was added Lithium diisopropyl amino (2.0 M, 8.16 mL, 3.00 eq) at 0° C. under N₂ atmosphere. The reaction mixture was stirred at 0° C. for 1 h. To the reaction mixture was added dimethyl formamide (10.0 mL) at 0° C. The reaction mixture was stirred at 25° C. for 1 hour, then poured into water (30.0 mL) and extracted with ethyl acetate (3×20.0 mL). The organic layers were combined, washed with brine (30.0 mL), dried with anhydrous Na₂SO₄, filtered, and the filtrate concentrated to give a residue. To the residue was added acetic acid (20.0 mL). The resulting mixture was stirred at 65° C. for 2 h, then concentrated under reduced pressure to give a residue. The residue was purified by silica gel column chromatography (petroleum ether/ethyl acetate=1/1) to give tert-butyl (2S,4S)-4-(2-chloro-5-oxo-1,6-naphthyridin-6-yl)-2-methyl-piperidine-1-carboxylate (1.00 g, 49%) as a solid. LCMS: (ES, m/z): 378.2 [M+H]⁺. ¹H NMR (400 MHz, METHANOL-d₄) δ ppm 8.58 (d, J=8.38 Hz, 1H) 7.79 (d, J=7.75 Hz, 1H) 7.51 (d, J=8.50 Hz, 1H) 6.74 (d, J=7.75 Hz, 1 H) 4.90-4.98 (m, 1H) 4.00-4.08 (m, 1H) 3.80-3.88 (m, 1H) 3.45 (ddd, J=14.07, 10.19, 6.25 Hz, 1H) 2.24-2.33 (m, 1H) 2.08-2.18 (m, 1H) 1.94 (ddd, J=12.63, 5.94, 3.69 Hz, 1H) 1.77 (dtd, J=12.71, 6.37, 6.37, 3.06 Hz, 1H) 1.50 (s, 9H) 1.29 (d, J=6.38 Hz, 3H).

### Synthesis of Intermediate G167

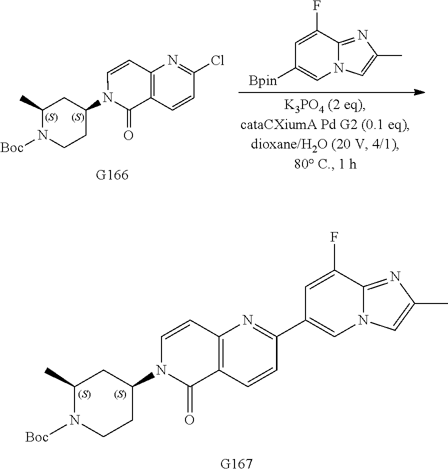

To a mixture of tert-butyl (2R,4R)-4-(2-chloro-5-oxo-1,6-naphthyridin-6-yl)-2-methyl-piperidine-1-carboxylate (150 mg, 396 umol, 1.00 eq) and 8-fluoro-2-methyl-6-(4,4,5,5-tetramethyl-1,3,2-dioxaborolan-2-yl)imidazo[1,2-a]pyridine (328 mg, 595 umol, 50% purity, 1.50 eq) in 1,4-dioxane (2.40 mL) and water (600 uL) was added K₃PO₄ (168 mg, 793 umol, 2.00 eq) and [2-(2-aminophenyl)phenyl]-chloro-palladium; bis(1-adamantyl)-butyl-phosphane (26.5 mg, 39.7 umol, 0.10 eq) at 25° C. under N₂ atmosphere. The reaction mixture was stirred at 80° C. for 1 h, then poured into water (20.0 mL) and extracted with ethyl acetate (3×20.0 mL). The organic layers were combined, washed with brine (50.0 mL), dried with anhydrous Na₂SO₄, filtered, and the filtrate concentrated to give a residue. The residue was purified by silica gel column chromatography(petroleum ether/ethyl acetate=1/1) to give tert-butyl (2R,4R)-4-[2-(8-fluoro-2-methyl-imidazo[1,2-a]pyridin-6-yl)-5-oxo-1,6-naphthyridin-6-yl]-2-methyl-piperidine-1-carboxylate (120 mg, 62%) as a solid. LCMS: (ES, m/z): 492.3 [M+H]⁺. ¹H NMR (400 MHz, METHANOL-d₄) δ ppm 9.15 (d, J=1.25 Hz, 1H) 8.69 (d, J=8.50 Hz, 1H) 8.03 (d, J=8.63 Hz, 1H) 7.93 (dd, J=12.13, 1.25 Hz, 1H) 7.82 (d, J=2.38 Hz, 1H) 7.77 (d, J=7.88 Hz, 1H) 6.93 (d, J=7.88 Hz, 1H) 4.97-5.04 (m, 1H) 4.05 (br dd, J=11.63, 5.88 Hz, 1H) 3.82-3.89 (m, 1H) 3.47 (dt, J=10.07, 3.97 Hz, 1H) 2.46 (s, 3H) 2.27-2.36 (m, 1H) 2.11-2.21 (m, 1H) 1.94-2.00 (m, 1H) 1.78-1.84 (m, 1H) 1.51 (s, 9H) 1.31 (d, J=6.25 Hz, 3H). ¹⁹F NMR (376 MHz, METHANOL-d₄) δ ppm−133.692 (s, 1F).

### Synthesis of Compound 1507

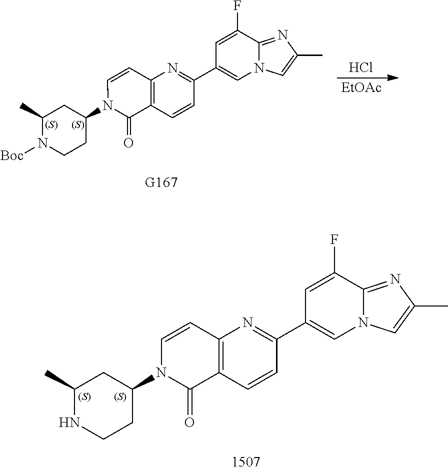

To a solution of tert-butyl (2S,4S)-4-[2-(8-fluoro-2-methyl-imidazo[1,2-a]pyridin-6-yl)-5-oxo-1,6-naphthyridin-6-yl]-2-methyl-piperidine-1-carboxylate (150 mg, 305 umol, 1.00 eq) in ethyl acetate (3.00 mL) was added HCl/ethyl acetate (4.0 M, 3.00 mL) at 25° C. The reaction mixture was stirred at 25° C. for 4 h, then concentrated to give a residue which was triturated with methyl tert-butyl ether (5.00 mL). The resulting mixture was filtered and the filter cake concentrated under reduced pressure to give 2-(8-fluoro-2-methyl-imidazo[1,2-a]pyridin-6-yl)-6-[(2S,4S)-2-methyl-4-piperidyl]-1,6-naphthyridin-5-one (72.8 mg, 61%) as a solid. LCMS: (ESI, m/z): 392.2 [M+H]⁺. ¹H NMR (400 MHz, METHANOL-d₄) δ ppm 9.54 (s, 1H) 8.81 (d, J=8.41 Hz, 1H) 8.67 (d, J=11.29 Hz, 1H) 8.14-8.23 (m, 2H) 7.74 (d, J=7.91 Hz, 1H) 6.99 (d, J=7.78 Hz, 1H) 5.10-5.18 (m, 1H) 3.62 (dt, J=11.11, 1.85 Hz, 1H) 3.53 (br s, 1H) 3.33 (br s, 1H) 2.63 (s, 3H) 2.32 (dd, J=12.80, 4.14 Hz, 1H) 2.19-2.27 (m, 2H) 2.17 (s, 1H) 1.45 (d, J=6.53 Hz, 3H). ¹⁹F NMR (376 MHz, METHANOL-d₄) δ ppm−133.943 (s, 1F).

### Example G92: Synthesis of Compound 1501

### Synthesis of Intermediate G168

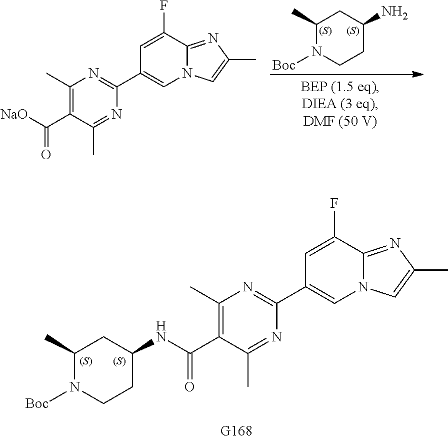

To a mixture of [2-(8-fluoro-2-methyl-imidazo[1,2-a]pyridin-6-yl)-4,6-dimethyl-pyrimidine-5-carbonyl]oxysodium (100 mg, 310 umol, 1.00 eq) and tert-butyl (2S,4S)-4-amino-2-methyl-piperidine-1-carboxylate (73.2 mg, 341 umol, 1.10 eq) in N,N-dimethyl formamide (5.00 mL) was added 2-bromo-1-ethyl-pyridin-1-ium; tetrafluoroborate (127 mg, 465 umol, 1.50 eq) and diisopropylamine (120 mg, 930 umol, 162 uL, 3.00 eq). The reaction mixture was stirred at 30° C. for 2 h, then cooled to 20° C. Three additional reactions were set up in parallel and the four reaction mixtures were combined and poured into water (100 mL). The resulting mixture was extracted with ethyl acetate (5×50.0 mL). The organic layers were combined, washed with brine (100 mL), dried over anhydrous Na₂SO₄, filtered, and the filtrate concentrated to give a residue. The residue was purified by silica gel column chromatography, eluted with 20%-50% of ethyl acetate in petroleum ether to give tert-butyl (2S,4S)-4-[[2-(8-fluoro-2-methyl-imidazo[1,2-a]pyridin-6-yl)-4,6-dimethyl-pyrimidine-5-carbonyl]amino]-2-methyl-piperidine-1-carboxylate (260 mg, 524 umol, 42%) as an oil. LCMS: (ESI, m/z):497.3 [M+H]⁺. ¹H NMR (400 MHz, DMSO-d₆) δ ppm 9.36 (d, J=1.25 Hz, 1H) 8.66 (d, J=7.15 Hz, 1H) 8.04 (d, J=2.51 Hz, 1H) 7.85 (dd, J=12.30, 1.25 Hz, 1H) 4.06-4.15 (m, 1H) 3.70-3.77 (m, 2H) 3.63 (br d, J=3.89 Hz, 1H) 2.47 (s, 6H) 2.38 (s, 3H) 1.84-1.94 (m, 2H) 1.71-1.78 (m, 2H) 1.40 (s, 9H) 1.21 (s, 3H). ¹⁹F NMR (376 MHz, DMSO-d₆) δ ppm−132.205 (s, 1F).

### Synthesis of Intermediate G169

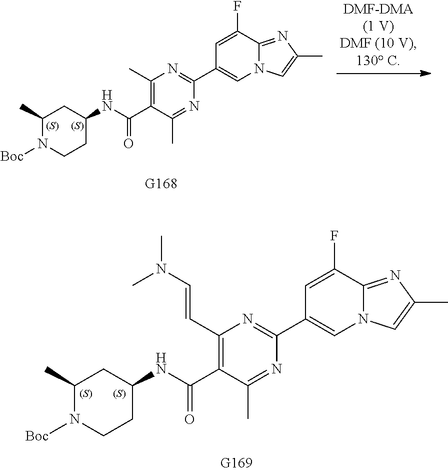

To a solution of tert-butyl (2S,4S)-4-[[2-(8-fluoro-2-methyl-imidazo[1,2-a]pyridin-6-yl)-4,6-dimethyl-pyrimidine-5-carbonyl]amino]-2-methyl-piperidine-1-carboxylate (250 mg, 503 umol, 1.00 eq) in N,N-dimethyl formamide (2.50 mL) was added 1,1-dimethoxy-N,N-dimethylmethanamine (250 uL, 3.74 eq). The reaction mixture was stirred at 130° C. for 2.5 h, then concentrated to give crude tert-butyl (2S,4S)-4-[[4-[(E)-2-(dimethylamino)vinyl]-2-(8-fluoro-2-methyl-imidazo[1,2-a]pyridin-6-yl)-6-methyl-pyrimidine-5-carbonyl]amino]-2-methyl-piperidine-1-carboxylate (240 mg, 86%) as an oil. LCMS: (ESI, m/z): 525.4 [M−Me₂NH+H₂O+H]⁺.

### Synthesis of Intermediate G170

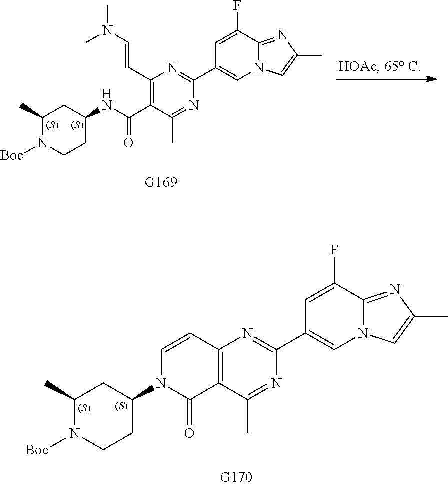

A solution of tert-butyl (2S,4S)-4-[[4-[(E)-2-(dimethylamino)vinyl]-2-(8-fluoro-2-methyl-imidazo[1,2-a]pyridin-6-yl)-6-methyl-pyrimidine-5-carbonyl]amino]-2-meth yl-piperidine-1-carboxylate (240 mg, 434 umol, 1.00 eq) in acetic acid (2.40 mL) was stirred at 65° C. for 2 h, then cooled to 20° C. The reaction mixture was concentrated to give a residue, which was purified by silica gel column chromatography, eluted with 40%-60% of ethyl acetate in petroleum ether to give tert-butyl (2S,4S)-4-[2-(8-fluoro-2-methyl-imidazo[1,2-a]pyridin-6-yl)-4-methyl-5-oxo-pyrido[4,3-d]pyrimidin-6-yl]-2-methyl-piperidine-1-carboxylate (60.0 mg, 27%) as a solid. LCMS: (ESI, m/z): 507.4 [M+H]⁺. ¹H NMR (400 MHz, DMSO-d₆) δ ppm 9.48 (d, J=1.25 Hz, 1H) 8.05-8.12 (m, 2H) 7.93 (d, J=12.17 Hz, 1H) 6.69 (d, J=7.78 Hz, 1H) 4.79-4.90 (m, 1H) 3.84-3.95 (m, 1H) 3.63-3.73 (m, 1H) 3.37-3.45 (m, 1H) 3.03 (s, 3H) 2.39 (s, 3H) 2.02-2.17 (m, 2H) 1.78-1.92 (m, 1H) 1.63-1.71 (m, 1H) 1.44 (s, 9H) 1.21 (d, J=6.27 Hz, 3H). ¹⁹F NMR (376 MHz, DMSO-d₆) δ ppm−132.103 (s, 1F).

### Synthesis of Compound 1501

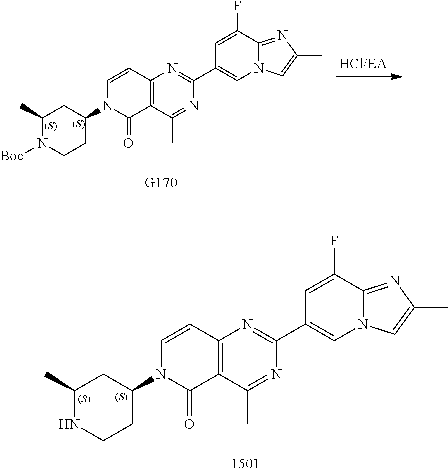

A solution of tert-butyl (2S,4S)-4-[2-(8-fluoro-2-methyl-imidazo[1,2-a]pyridin-6-yl)-4-methyl-5-oxo-pyrido[4,3-d]pyrimidin-6-yl]-2-methyl-piperidine-1-carboxylate (60.0 mg, 118 umol, 1.00 eq) in HCl (gas)/ethyl acetate (2.0 M, 20.0 mL) was stirred at 25° C. for 2 h, then concentrated to give a residue which was triturated with dichloromethane (3.00 mL). The resulting mixture was filtered and the filter cake dried to give 2-(8-fluoro-2-methyl-imidazo[1,2-a]pyridin-6-yl)-4-methyl-6-[(2S,4S)-2-methyl-4-piperidyl]pyrido[4,3-d]pyrimidin-5-one (37.2 mg, 77%) as a solid. LCMS: (ESI, m/z): 407.2 [M+H]⁺. ¹H NMR (400 MHz, METHANOL-d₄) δ ppm 9.77 (s, 1H) 8.77 (d, J=11.01 Hz, 1H) 8.24 (s, 1H) 7.91 (d, J=7.63 Hz, 1H) 6.84 (d, J=7.63 Hz, 1H) 5.05-5.17 (m, 1H) 3.62 (brd, J=12.13 Hz, 1H) 3.47-3.57 (m, 1H) 3.25-3.30 (m, 1H) 3.14 (s, 3H) 2.63 (s, 3H) 2.04-2.32 (m, 4H) 1.44 (d, J=6.50 Hz, 3H). ¹⁹F NMR (376 MHz, METHANOL-d₄) δ ppm−134.232 (s, 1F).

### Example G93: Synthesis of Compound 1505 Synthesis of Intermediate G171

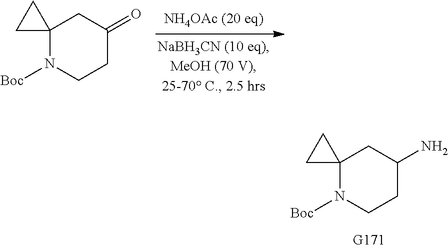 [SMILES: CC(C)(C)OC(=O)N1CCC(=O)CC12CC2 ; CC(C)(C)OC(=O)N1CCC(N)CC12CC2]

To a solution of tert-butyl 7-oxo-4-azaspiro[2.5]octane-4-carboxylate (3.00 g, 13.3 mmol, 1.00 eq) in methanol (210 mL) was added ammonium acetate (20.5 g, 266 mmol, 20.0 eq) at 25° C. under N₂ atmosphere. The reaction mixture was stirred at 70° C. for 30 min. To the reaction mixture was added sodium cyanoborohydride (8.37 g, 133 mmol, 10.0 eq) at 70° C. under N₂ atmosphere. The reaction mixture was stirred at 70° C. for an additional 2 h, then cooled to 25° C., quenched with saturated sodium bicarbonate solution (400 mL) and extracted with ethyl acetate (3×400 mL). The organic layers were combined, washed with brine (400 mL), dried over Na₂SO₄, filtered, and the filtrate concentrated under reduced pressure to give tert-butyl 7-amino-4-azaspiro[2.5]octane-4-carboxylate (4 g, 93%) as an oil. ¹H NMR (400 MHz, CHLOROFORM-d) δ ppm 4.07 (br d, J=12.55 Hz, 1H) 3.60-3.72 (m, 1H) 2.89-3.07 (m, 1H) 2.10-2.24 (m, 2H) 1.47 (s, 9H) 1.21-1.33 (m, 2H) 0.96-1.04 (m, 1H) 0.77-0.95 (m, 1H) 0.59-0.65 (m, 2H).

### Synthesis of Intermediate G172

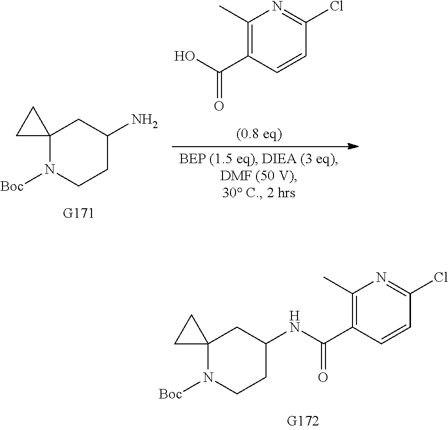 [SMILES: Cc1nc(Cl)ccc1C(=O)O ; CC(C)(C)OC(=O)N1CCC(N)CC12CC2 ; Cc1nc(Cl)ccc1C(=O)NC1CCN(C(=O)OC(C)(C)C)C2(CC2)C1]

To a mixture of 6-chloro-2-methyl-pyridine-3-carboxylic acid (727 mg, 4.24 mmol, 0.80 eq) and tert-butyl 7-amino-4-azaspiro[2.5]octane-4-carboxylate (1.20 g, 5.30 mmol, 1.00 eq) in N,N-dimethyl formamide (6.00 mL) was added 2-bromo-1-ethyl-pyridin-1-ium; tetrafluoroborate (2.18 g, 7.95 mmol, 1.50 eq) and N,N-diisopropylethylamine (2.06 g, 15.9 mmol, 2.77 mL, 3.00 eq) at 25° C. under N₂ atmosphere. The reaction mixture was stirred at 30° C. for 2 h, then poured into water (40.0 mL) and extracted with ethyl acetate (3×40.0 mL). The organic layers were combined, washed with brine (100.0 mL), dried over anhydrous Na₂SO₄, filtered, and the filtrate concentrated to give a residue. The residue was purified by silica gel column chromatography (petroleum ether/ethyl acetate=2/1) to give tert-butyl 7-[(6-chloro-2-methyl-pyridine-3-carbonyl)amino]-4-azaspiro[2.5]octane-4-carboxylate (1.00 g, 50%) as an oil. LCMS: (ESI, m/z): 324.2 [M−55]⁺. ¹H NMR (400 MHz, METHANOL-d₄) δ ppm 7.71 (d, J=8.16 Hz, 1H) 7.32 (d, J=8.03 Hz, 1H) 4.20-4.33 (m, 1H) 3.99-4.05 (m, 1H) 3.00-3.10 (m, 1H) 2.54 (s, 3H) 1.87-2.00 (m, 2H) 1.47 (s, 9H) 1.39-1.45 (m, 1H) 1.35 (dd, J=12.23, 3.83 Hz, 1H) 1.22-1.26 (m, 1H) 0.87-0.94 (m, 1H) 0.58-0.69 (m, 2H).

### Synthesis of Intermediate G173

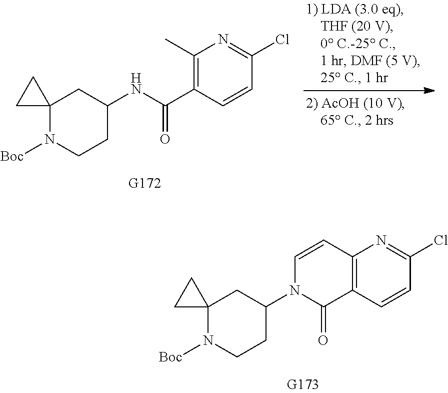 [SMILES: Cc1nc(Cl)ccc1C(=O)NC1CCN(C(=O)OC(C)(C)C)C2(CC2)C1 ; CC(C)(C)OC(=O)N1CCC(n2ccc3nc(Cl)ccc3c2=O)CC12CC2]

To a solution of tert-butyl 7-[(6-chloro-2-methyl-pyridine-3-carbonyl)amino]-4-azaspiro[2.5]octane-4-carboxylate (1.00 g, 2.63 mmol, 1.00 eq) in tetrahydrofuran (20.0 mL) was added LDA (2.0 M, 3.95 mL, 3.00 eq) at 0° C. The reaction mixture was stirred at 0° C. for 1 h. To the reaction mixture was added dimethyl formamide (5.00 mL) at 0° C. The resulting mixture was stirred at 25° C. for 1 h, then poured into water (30.0 mL) and extracted with ethyl acetate (3×30.0 mL). The organic layers were combined, washed with brine (100 mL), dried over anhydrous Na₂SO₄, filtered, and the filtrate was concentrated under reduced pressure to give a residue. To the residue was added acetic acid (10.0 mL) and the resulting mixture was stirred at 65° C. for 2 h, then concentrated under reduced pressure to give a residue. The residue was purified by silica gel column chromatography (petroleum ether/ethyl acetate=1/1) to give tert-butyl 7-(2-chloro-5-oxo-1,6-naphthyridin-6-yl)-4-azaspiro[2.5]octane-4-carboxylate (500 mg, 49%) as a solid. LCMS: (ESI, m/z): 390.3 [M+H]⁺. ¹H NMR (400 MHz, METHANOL-d₄) δ ppm 8.57-8.62 (m, 1H) 7.73 (d, J=7.75 Hz, 1H) 7.51 (d, J=8.50 Hz, 1H) 6.73 (d, J=7.75 Hz, 1H) 5.23 (tt, J=12.07, 4.25 Hz, 1H) 4.19 (dt, J=13.48, 3.33 Hz, 1H) 3.07-3.19 (m, 1H) 2.37 (br t, J=12.26 Hz, 1H) 1.85-1.95 (m, 2H) 1.51 (s, 9H) 1.25-1.39 (m, 2H) 0.96 (dt, J=9.54, 6.55 Hz, 1H) 0.64-0.74 (m, 2H).

### Synthesis of Intermediate G174

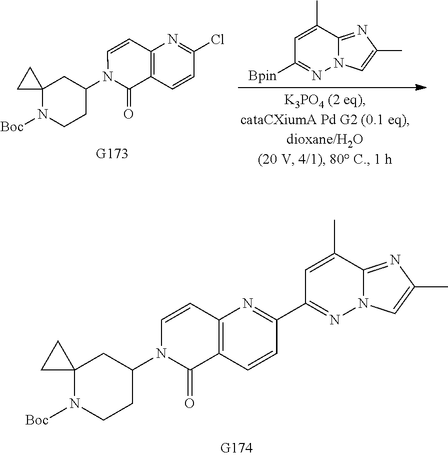

To a mixture of tert-butyl 7-(2-chloro-5-oxo-1,6-naphthyridin-6-yl)-4-azaspiro[2.5]octane-4-carboxylate (300 mg, 769 umol, 1.00 eq) and 2,8-dimethyl-6-(4,4,5,5-tetramethyl-1,3,2-dioxaborolan-2-yl)imidazo[1,2-b]pyridazine (840 mg, 3.08 mmol, 4.00 eq) in 1,4-dioxane (4.80 mL) and water (1.20 mL) was added K₃PO₄ (327 mg, 1.54 mmol, 2.00 eq) and [2-(2-aminophenyl)phenyl]-chloro-palladium; bis(1-adamantyl)-butyl-phosphane (51.4 mg, 76.9 umol, 0.10 eq) at 25° C. under N₂ atmosphere. The reaction mixture was stirred at 80° C. for 1 h, then cooled to 25° C., poured into water (20.0 mL) and extracted with ethyl acetate (3×20.0 mL). The organic layers were combined, washed with brine (50.0 mL), dried over anhydrous Na₂SO₄, filtered, and the filtrate concentrated to give a residue. The residue was purified by silica gel column chromatography (petroleum ether/ethyl acetate=1/1) to give tert-butyl 7-[2-(2,8-dimethylimidazo[1,2-b]pyridazin-6-yl)-5-oxo-1,6-naphthyridin-6-yl]-4-azaspiro[2.5]octane-4-carboxylate (250 mg, 65%) as a solid. LCMS: (ESI, m/z): 501.3 [M+H]^{+ 1}H NMR (400 MHz, METHANOL-d₄) δ ppm 8.74 (d, J=8.03 Hz, 1H) 8.45 (d, J=8.53 Hz, 1H) 8.18 (d, J=1.13 Hz, 1H) 7.98 (d, J=0.75 Hz, 1H) 7.72 (d, J=7.78 Hz, 1H) 6.97 (d, J=7.78 Hz, 1H) 5.23-5.37 (m, 1H) 4.14-4.28 (m, 1H) 3.11-3.18 (m, 1H) 2.70 (d, J=1.00 Hz, 3H) 2.51 (d, J=0.63 Hz, 3H) 2.41 (br t, J=12.05 Hz, 1H) 1.90-1.99 (m, 2H) 1.53 (s, 9H) 1.27-1.37 (m, 2H) 0.98 (dt, J=9.38, 6.48 Hz, 1H) 0.65-0.76 (m, 2H).

### Synthesis of Intermediate G175 and G176

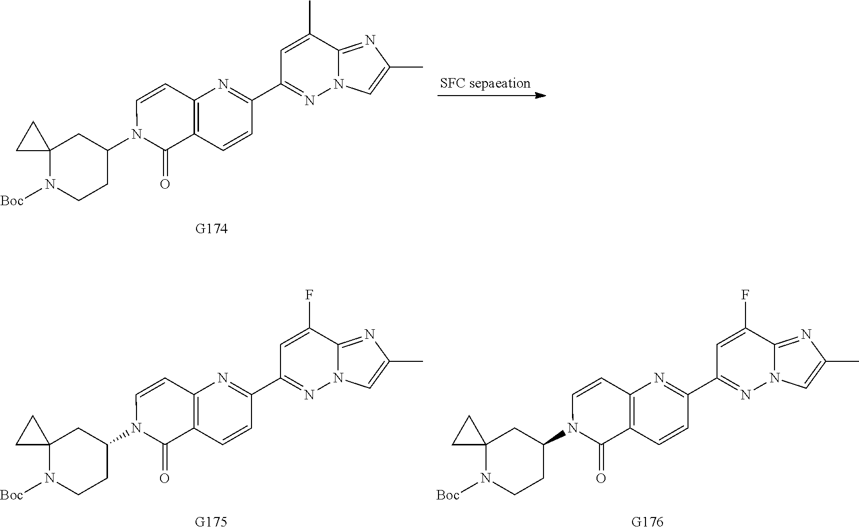 [SMILES: Cc1cn2nc(-c3ccc4c(=O)n(C5CCN(C(=O)OC(C)(C)C)C6(CC6)C5)ccc4n3)cc(C)c2n1 ; Cc1cn2nc(-c3ccc4c(=O)n([C@@H]5CCN(C(=O)OC(C)(C)C)C6(CC6)C5)ccc4n3)cc(F)c2n1 ; Cc1cn2nc(-c3ccc4c(=O)n([C@H]5CCN(C(=O)OC(C)(C)C)C6(CC6)C5)ccc4n3)cc(F)c2n1]

7-[2-(2,8-dimethylimidazo[1,2-b]pyridazin-6-yl)-5-oxo-1,6-naphthyridin-6-yl]-4-azaspiro[2.5]octane-4-carboxylate (250 mg) was purified by SFC (Condition G3) to give tert-butyl (7R)-7-[2-(2,8-dimethylimidazo[1,2-b]pyridazin-6-yl)-5-oxo-1,6-naphthyridin-6-yl]-4-azaspiro[2.5]octane-4-carboxylate (90.0 mg, 45%) and tert-butyl (7S)-7-[2-(2,8-dimethylimidazo[1,2-b]pyridazin-6-yl)-5-oxo-1,6-naphthyridin-6-yl]-4-azaspiro[2.5]octane-4-carboxylate (90.0 mg, 45%) as a solid. G175: LCMS: (ESI, m/z): 501.4 [M+H]⁺. ¹H NMR (400 MHz, METHANOL-d₄) δ ppm 8.74 (d, J=8.31 Hz, 1H) 8.45 (d, J=8.31 Hz, 1H) 8.18 (s, 1H) 7.99 (s, 1H) 7.73 (d, J=7.83 Hz, 1H) 6.97 (d, J=7.34 Hz, 1H) 5.24-5.34 (m, 1H) 4.18-4.24 (m, 1H) 3.11-3.18 (m, 1H) 2.71 (s, 3H) 2.51 (s, 3H) 2.42 (br t, J=12.72 Hz, 1H) 1.91-1.98 (m, 2H) 1.53 (s, 9H) 1.27-1.36 (m, 2H) 0.95-1.00 (m, 1H) 0.67-0.76 (m, 2H). G176: LCMS: (ESI, m/z): 501.4 [M+H]⁺. ¹H NMR (400 MHz, METHANOL-d₄) δ ppm 8.74 (d, J=8.44 Hz, 1H) 8.45 (d, J=8.56 Hz, 1H) 8.18 (d, J=0.98 Hz, 1H) 7.99 (s, 1H) 7.73 (d, J=7.82 Hz, 1H) 6.97 (d, J=7.82 Hz, 1H) 5.25-5.33 (m, 1H) 4.16-4.28 (m, 1H) 3.09-3.21 (m, 1H) 2.70 (s, 3H) 2.51 (s, 3H) 2.41 (br t, J=12.04 Hz, 1H) 1.92-2.00 (m, 2H) 1.53 (s, 9H) 1.27-1.36 (m, 2H) 0.98 (dt, J=9.60, 6.57 Hz, 1H) 0.67-0.76 (m, 2H).

### Synthesis of Compound 1505

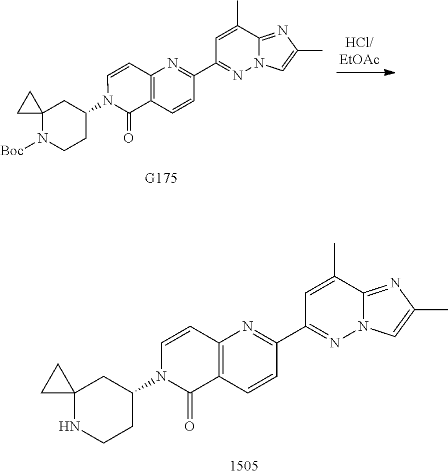 [SMILES: Cc1cn2nc(-c3ccc4c(=O)n([C@@H]5CCN(C(=O)OC(C)(C)C)C6(CC6)C5)ccc4n3)cc(C)c2n1 ; Cc1cn2nc(-c3ccc4c(=O)n([C@@H]5CCNC6(CC6)C5)ccc4n3)cc(C)c2n1]

To a solution of tert-butyl (7R)-7-[2-(2,8-dimethylimidazo[1,2-b]pyridazin-6-yl)-5-oxo-1,6-naphthyridin-6-yl]-4-azaspiro[2.5]octane-4-carboxylate (90.0 mg, 179 umol, 1.00 eq) in ethyl acetate (1.80 mL) was added HCl/ethyl acetate (4.0 M, 1.80 mL) at 25° C. The reaction mixture was stirred at 25° C. for 4 h, then concentrated under reduced pressure to give a residue. The residue was triturated with methyl tert-butyl ether (5.00 mL) to form a suspension. The resulting suspension was filtered and the filter cake concentrated under reduced pressure to give 6-[(7R)-4-azaspiro[2.5]octan-7-yl]-2-(2,8-dimethylimidazo[1,2-b]pyridazin-6-yl)-1,6-naphthyridin-5-one (72.2 mg, 92%) as a solid. LCMS: (ESI, m/z): 401.2 [M+H]⁺. ¹H NMR (400 MHz, METHANOL-d₄) δ ppm 8.86 (d, J=8.53 Hz, 1H) 8.80 (d, J=1.13 Hz, 1H) 8.56 (d, J=8.53 Hz, 1H) 8.41 (d, J=0.88 Hz, 1H) 7.79 (d, J=7.78 Hz, 1H) 7.04 (d, J=7.91 Hz, 1H) 5.16-5.27 (m, 1H) 3.59-3.67 (m, 1H) 3.42 (td, J=12.86, 3.14 Hz, 1H) 2.84 (d, J=0.88 Hz, 3H) 2.80 (s, 1H) 2.68 (d, J=0.75 Hz, 3H) 2.44 (br dd, J=12.74, 3.83 Hz, 1H) 2.28 (br d, J=12.92 Hz, 1H) 1.60-1.68 (m, 1H) 1.12-1.20 (m, 2H) 0.97-1.08 (m, 2H).

### Synthesis of Compound 1506

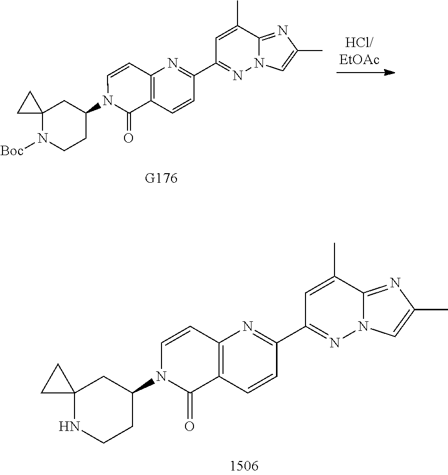 [SMILES: Cc1cn2nc(-c3ccc4c(=O)n([C@H]5CCN(C(=O)OC(C)(C)C)C6(CC6)C5)ccc4n3)cc(C)c2n1 ; Cc1cn2nc(-c3ccc4c(=O)n([C@H]5CCNC6(CC6)C5)ccc4n3)cc(C)c2n1]

To a solution of tert-butyl (7S)-7-[2-(2,8-dimethylimidazo[1,2-b]pyridazin-6-yl)-5-oxo-1,6-naphthyridin-6-yl]-4-azaspiro[2.5]octane-4-carboxylate (90.0 mg, 179 umol, 1.00 eq) in ethyl acetate (1.80 mL) was added HCl/ethyl acetate (4.0 M, 1.80 mL) at 25° C. The reaction mixture was stirred at 25° C. for 4 h, then concentrated under reduced pressure to give a residue. The residue was triturated with methyl tert-butyl ether (5.00 mL) to form a suspension. The suspension was filtered, and the filter cake was concentrated under reduced pressure to give 6-[(7S)-4-azaspiro[2.5]octan-7-yl]-2-(2,8-dimethylimidazo[1,2-b]pyridazin-6-yl)-1,6-naphthyridin-5-one (71.5 mg, 99%) as a solid. LCMS: (ESI, m/z): 401.3 [M+H]⁺. ¹H NMR (400 MHz, METHANOL-d₄) δ ppm 8.86 (d, J=8.41 Hz, 1H) 8.80 (d, J=1.13 Hz, 1H) 8.56 (d, J=8.41 Hz, 1H) 8.41 (d, J=1.00 Hz, 1H) 7.79 (d, J=7.78 Hz, 1H) 7.04 (d, J=7.78 Hz, 1H) 5.18-5.26 (m, 1H) 3.60-3.67 (m, 1H) 3.42 (td, J=12.83, 3.07 Hz, 1H) 2.84 (d, J=0.88 Hz, 3H) 2.80 (s, 1H) 2.68 (d, J=0.75 Hz, 3H) 2.37-2.47 (m, 1H) 2.28 (br d, J=13.43 Hz, 1H) 1.60-1.68 (m, 1H) 1.17 (s, 2H) 1.03 (br d, J=14.56 Hz, 2H).

### Example G94: Synthesis of Compound 1507

### Synthesis of Intermediate G177

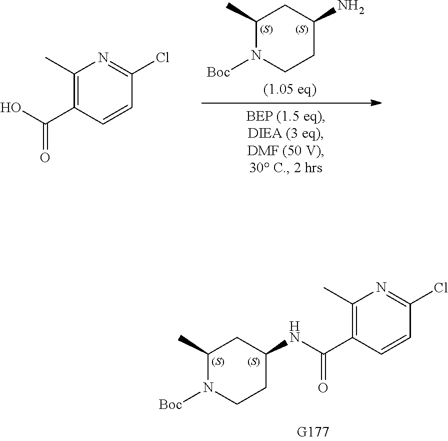

To a mixture of 6-chloro-2-methyl-pyridine-3-carboxylic acid (1.50 g, 8.74 mmol, 1.00 eq) and tert-butyl (2S,4S)-4-amino-2-methyl-piperidine-1-carboxylate (1.97 g, 9.18 mmol, 1.05 eq) in N,N-dimethyl formamide (75.0 mL) was added 2-bromo-1-ethyl-pyridin-1-ium; tetrafluoroborate (3.59 g, 13.1 mmol, 1.50 eq) and N,N-diisopropylethylamine (3.39 g, 26.2 mmol, 4.57 mL, 3.00 eq) at 25° C. under N₂ atmosphere. The reaction mixture was stirred at 30° C. for 2 h, then poured into water (30.0 mL) and extracted with ethyl acetate (3×20.0 mL). The organic layers were combined, washed with brine (30.0 mL), dried with anhydrous Na₂SO₄, filtered, and the filtrate was concentrated under reduced pressure to give a residue. The residue was purified by silica gel column chromatography (petroleum ether/ethyl acetate=2/1) to give tert-butyl (2S,4S)-4-[(6-chloro-2-methyl-pyridine-3-carbonyl)amino]-2-methyl-piperidine-1-carboxylate (1.50 g, 47%) as an oil. LCMS: (ESI, m/z): 368.3 [M+H]⁺. ¹H NMR (400 MHz, METHANOL-d₄) δ ppm 7.73 (d, J=8.03 Hz, 1H) 7.34 (d, J=8.03 Hz, 1H) 4.08-4.16 (m, 2H) 3.71-3.81 (m, 1H) 3.24-3.30 (m, 1H) 2.55 (s, 3H) 2.02-2.12 (m, 1H) 1.93-2.00 (m, 1H) 1.81 (dt, J=13.90, 7.04 Hz, 1H) 1.59-1.68 (m, 1H) 1.47 (s, 9H) 1.27 (d, J=6.78 Hz, 3H).

### Synthesis of Intermediate G178

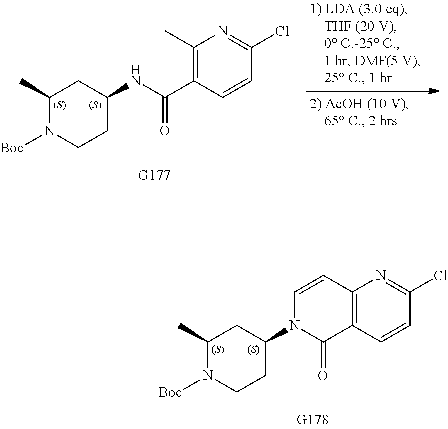

To a solution of tert-butyl (2S,4S)-4-[(6-chloro-2-methyl-pyridine-3-carbonyl)amino]-2-methyl-piperidine-1-carboxylate (0162-1, 2.00 g, 5.44 mmol, 1.00 eq) in tetrahydrofuran (30.0 mL) was added di(propan-2-yl) azanide (2.0 M, 8.16 mL, 3.00 eq) at 0° C. under N₂ atmosphere. The reaction mixture was stirred at 0° C. for 1 h. To the reaction mixture was added dimethyl formamide (7.50 mL) at 0° C. The resulting mixture was stirred at 25° C. for 1 h, then poured into water (30.0 mL) and extracted with ethyl acetate (3×30.0 mL). The organic layers were combined, washed with brine (30.0 mL), dried with anhydrous Na₂SO₄, filtered, and the filtrate concentrated to give a residue. To the residue was added acetic acid (20.0 mL) and the resulting mixture was stirred at 65° C. for 6 h, then concentrated under reduced pressure to give a residue. The residue was purified by silica gel column chromatography (petroleum ether/ethyl acetate=1/1) to give tert-butyl (2S,4S)-4-(2-chloro-5-oxo-1,6-naphthyridin-6-yl)-2-methyl-piperidine-1-carboxylate (1.00 g, 49%) as a solid. LCMS: (ESI, m/z): 378.2 [M+H]⁺. ¹H NMR (400 MHz, METHANOL-d₄) δ ppm 8.58 (d, J=8.38 Hz, 1H) 7.79 (d, J=7.75 Hz, 1H) 7.51 (d, J=8.50 Hz, 1H) 6.74 (d, J=7.75 Hz, 1H) 4.90-4.98 (m, 1H) 4.00-4.08 (m, 1H) 3.80-3.88 (m, 1H) 3.45 (ddd, J=14.07, 10.19, 6.25 Hz, 1H) 2.24-2.33 (m, 1H) 2.08-2.18 (m, 1H) 1.94 (ddd, J=12.63, 5.94, 3.69 Hz, 1H) 1.77 (dtd, J=12.71, 6.37, 6.37, 3.06 Hz, 1H) 1.50 (s, 9H) 1.29 (d, J=6.38 Hz, 3H).

### Synthesis of Intermediate G179

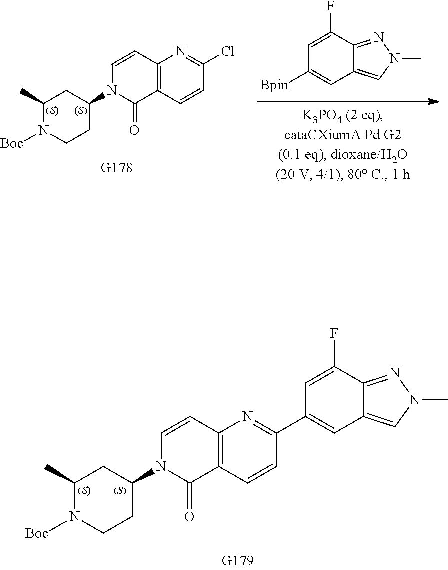

To a mixture of tert-butyl (2S,4S)-4-(2-chloro-5-oxo-1,6-naphthyridin-6-yl)-2-methyl-piperidine-1-carboxylate (200 mg, 529 umol, 1.00 eq) and 7-fluoro-2-methyl-5-(4,4,5,5-tetramethyl-1,3,2-dioxaborolan-2-yl)indazole (146 mg, 529 umol, 1.00 eq) in 1,4-dioxane (3.20 mL) and water (800 uL) was added [2-(2-aminophenyl)phenyl]-chloro-palladium; bis(1-adamantyl)-butyl-phosphane (35.3 mg, 52.9 umol, 0.10 eq) and K₃PO₄ (224 mg, 1.06 mmol, 2.00 eq) at 25° C. The reaction mixture was stirred at 80° C. for 1 h, then poured into water (20.0 mL) and extracted with ethyl acetate (3×20.0 mL). The organic layers were combined, washed with brine (50.0 mL), dried over anhydrous Na₂SO₄, filtered, and the filtrate concentrated to give a residue. The residue was purified by silica gel column chromatography (petroleum ether/ethyl acetate=1/1) to give tert-butyl(2S,4S)-4-[2-(7-fluoro-2-methyl-indazol-5-yl)-5-oxo-1,6-naphthyridin-6-yl]-2-methyl-piperidine-1-carboxylate (150 mg, 58%) as a solid. LCMS: (ESI, m/z): 492.3 [M+H]⁺. ¹H NMR (400 MHz, METHANOL-d₄) δ ppm 8.67 (d, J=8.53 Hz, 1H) 8.47 (d, J=2.64 Hz, 1H) 8.38 (s, 1H) 8.08 (d, J=8.53 Hz, 1H) 7.89-7.96 (m, 1H) 7.76 (d, J=7.78 Hz, 1H) 6.95 (d, J=7.78 Hz, 1H) 4.96-5.03 (m, 1H) 4.28 (s, 3H) 4.00-4.11 (m, 1H) 3.86 (ddd, J=14.18, 7.47, 2.57 Hz, 1H) 3.44-3.51 (m, 1H) 2.27-2.38 (m, 1H) 2.09-2.22 (m, 1H) 1.92-2.02 (m, 1H) 1.75-1.88 (m, 1H) 1.51 (s, 9H) 1.31 (d, J=6.27 Hz, 3H). ¹⁹F NMR (376 MHz, METHANOL-d₄) δ ppm−131.042 (s, 1F).

### Synthesis of Compound 1507

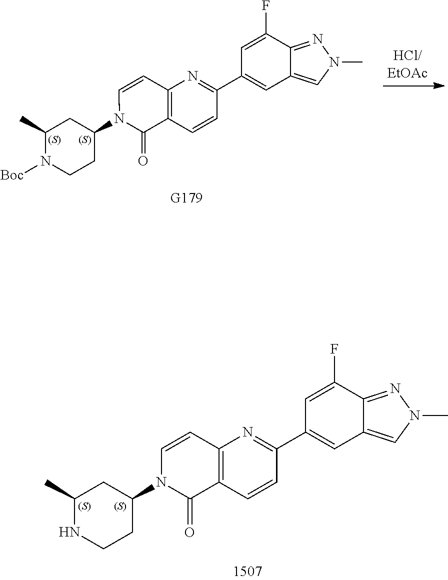

To a solution of tert-butyl (2S,4S)-4-[2-(7-fluoro-2-methyl-indazol-5-yl)-5-oxo-1,6-naphthyridin-6-yl]-2-methyl-piperidine-1-carboxylate (150 mg, 305 umol, 1.00 eq) in ethyl acetate (3.00 mL) was added HCl/ethyl acetate (4.0 M, 3.00 mL) at 25° C. The reaction mixture was stirred at 25° C. for 4 h, then concentrated under reduced pressure to give a residue. The residue was triturated with methyl tert-butyl ether (5.00 mL) to form a suspension. The resulting suspension was filtered and the filter cake concentrated under reduced pressure to give 2-(7-fluoro-2-methyl-indazol-5-yl)-6-[(2S,4S)-2-methyl-4-piperidyl]-1,6-naphthyridin-5-one (130 mg, 100%) as a solid. LCMS: (ESI, m/z): 392.3 [M+H]⁺. ¹H NMR (400 MHz, METHANOL-d₄) δ ppm 8.86 (d, J=8.66 Hz, 1H) 8.54 (d, J=2.51 Hz, 1H) 8.41 (d, J=1.25 Hz, 1H) 8.18 (d, J=8.66 Hz, 1H) 7.87 (dd, J=12.74, 1.32 Hz, 1H) 7.78 (d, J=7.78 Hz, 1H) 7.00 (d, J=7.65 Hz, 1H) 5.06-5.15 (m, 1H) 4.30 (s, 3H) 3.59-3.66 (m, 1H) 3.49-3.57 (m, 1H) 3.33 (br s, 1H) 2.27 (br s, 1H) 2.23 (br d, J=3.26 Hz, 2H) 2.08-2.17 (m, 1H) 1.44 (d, J=6.53 Hz, 3H) ¹⁹F NMR (376 MHz, METHANOL-d₄) δ ppm−130.082 (s, 1F).

### Example G95: Synthesis of Compound 1508 and 1509

### Synthesis of Intermediate G180

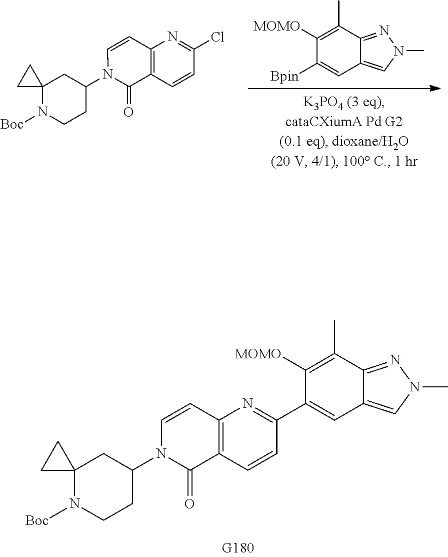

To a mixture of tert-butyl 7-(2-chloro-5-oxo-1,6-naphthyridin-6-yl)-4-azaspiro[2.5]octane-4-carboxylate (250 mg, 641 umol, 1.00 eq) and 6-(methoxymethoxy)-2,7-dimethyl-5-(4,4,5,5-tetramethyl-1,3,2-dioxaborolan-2-yl)indazole (533 mg, 1.60 mmol, 2.50 eq) in a mixture of 1,4-dioxane (4.00 mL) and water (1.00 mL) was added [2-(2-aminophenyl)phenyl]-chloro-palladium; bis(1-adamantyl)-butyl-phosphane (42.9 mg, 64.1 umol, 0.10 eq) and K₃PO₄ (408 mg, 1.92 mmol, 3.00 eq) under N₂ atmosphere. The reaction mixture was stirred at 100° C. for 1 h, then cooled to 25° C., filtered, and the filtrate was concentrated to give a residue which was purified by silica gel column chromatography (petroleum ether/ethyl acetate=1/10 to ½ over 40 min.) to give tert-butyl 7-[2-[6-(methoxymethoxy)-2,7-dimethyl-indazol-5-yl]-5-oxo-1,6-naphthyridin-6-yl]-4-azaspiro[2.5]octane-4-carboxylate (250 mg, 58%) as an oil. LCMS: (ESI, m/z): 560.3 [M+H]⁺. ¹H NMR (400 MHz, DMSO-d₆) δ ppm 8.53 (d, J=8.50 Hz, 1H) 8.42 (s, 1H) 7.89 (s, 1H) 7.81 (d, J=8.38 Hz, 1H) 7.77 (d, J=7.75 Hz, 1H) 6.77 (d, J=7.75 Hz, 1H) 5.05-5.20 (m, 1H) 4.68 (s, 2H) 4.19 (s, 3H) 4.04-4.09 (m, 1H) 3.30 (br d, J=2.00 Hz, 1H) 3.12 (s, 3H) 2.93-3.05 (m, 1H) 2.51-2.51 (m, 3H) 2.30 (br t, J=12.01 Hz, 1H) 1.83 (br d, J=3.13 Hz, 2H) 1.46 (s, 9H) 1.21-1.26 (m, 1H) 0.91 (dt, J=9.57, 6.16 Hz, 1H) 0.53-0.66 (m, 2H).

### Synthesis of Intermediate G181 and G182

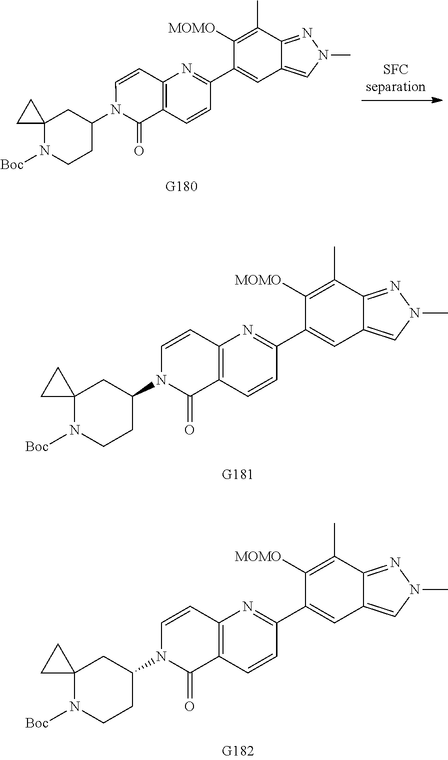

Tert-butyl 7-[2-[6-(methoxymethoxy)-2,7-dimethyl-indazol-5-yl]-5-oxo-1,6-naphthyridin-6-yl]-4-azaspiro[2.5]octane-4-carboxylate (168-1, 250 mg) was purified by SFC (Condition G3) to give tert-butyl (7S)-7-[2-[6-(methoxymethoxy)-2,7-dimethyl-indazol-5-yl]-5-oxo-1,6-naphthyridin-6-yl]-4-azaspiro[2.5]octane-4-carboxylate (100 mg, 40%) and tert-butyl (7R)-7-[2-[6-(methoxymethoxy)-2,7-dimethyl-indazol-5-yl]-5-oxo-1,6-naphthyridin-6-yl]-4-azaspiro[2.5]octane-4-carboxylate (100 mg, 40%) as solids. G181: ¹H NMR (400 MHz, DMSO-d₆) δ ppm 8.53 (d, J=8.53 Hz, 1H) 8.42 (s, 1H) 7.89 (s, 1H) 7.79 (dd, J=15.87, 8.09 Hz, 2H) 6.77 (d, J=7.65 Hz, 1H) 5.07-5.20 (m, 1H) 4.68 (s, 2H) 4.19 (s, 3H) 3.97-4.09 (m, 1H) 3.12 (s, 3H) 2.90-3.05 (m, 1H) 2.51 (br s, 3H) 2.29-2.37 (m, 1H) 1.79-1.87 (m, 2H) 1.46 (s, 9H) 1.16-1.25 (m, 2H) 0.91 (dt, J=9.82, 6.57 Hz, 1H) 0.50-0.66 (m, 2H). G182: ¹H NMR (400 MHz, DMSO-d₆) δ ppm 8.53 (d, J=8.41 Hz, 1H) 8.42 (s, 1H) 7.89 (s, 1H) 7.79 (dd, J=15.87, 8.22 Hz, 2H) 6.77 (d, J=7.65 Hz, 1H) 5.04-5.22 (m, 1H) 4.60-4.76 (m, 2H) 4.19 (s, 3H) 3.96-4.11 (m, 1H) 3.12 (s, 3H) 2.92-3.05 (m, 1H) 2.51-2.51 (m, 3H) 2.27-2.37 (m, 1H) 1.76-1.90 (m, 2H) 1.46 (s, 9H) 1.14-1.26 (m, 2H) 0.91 (dt, J=9.72, 6.31 Hz, 1H) 0.50-0.71 (m, 2H).

### Synthesis of Compound 1508

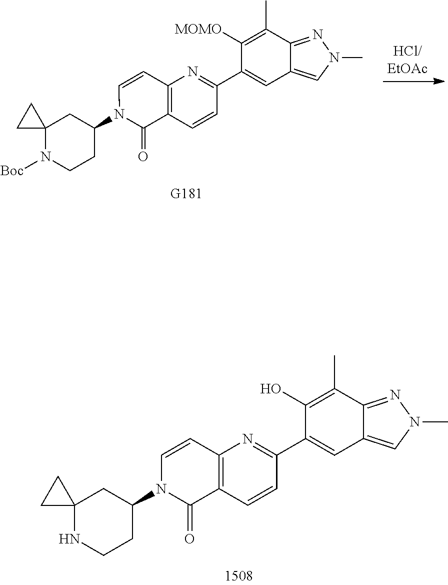

A solution of tert-butyl (7S)-7-[2-[6-(methoxymethoxy)-2,7-dimethyl-indazol-5-yl]-5-oxo-1,6-naphthyridin-6-yl]-4-azaspiro[2.5]octane-4-carboxylate (100 mg, 179 umol, 1.00 eq) in HCl (gas)/ethyl acetate (2.0 M, 20.0 mL) was stirred at 20° C. for 2 h, then concentrated under reduced pressure to give a residue. The residue was triturated with dichloromethane (10.0 mL) to give a suspension. The resulting suspension was filtered and the filter cake dried to give 6-[(7S)-4-azaspiro[2.5]octan-7-yl]-2-(6-hydroxy-2,7-dimethyl-indazol-5-yl)-1,6-naphthyridin-5-one (73.6 mg, 91%) as a solid. LCMS: (ESI, m/z): 416.2 [M+H]⁺. ¹H NMR (400 MHz, METHANOL-d₄) δ ppm 8.57 (d, J=8.76 Hz, 1H) 8.40-8.50 (m, 2H) 8.15 (d, J=9.01 Hz, 1H) 7.80 (d, J=7.88 Hz, 1H) 6.85 (d, J=7.75 Hz, 1H) 5.21 (ddd, J=12.13, 8.32, 3.81 Hz, 1H) 4.25 (s, 3H) 3.65 (br d, J=12.76 Hz, 1H) 3.40 (td, J=12.79, 3.06 Hz, 1H) 2.79 (t, J=12.88 Hz, 1H) 2.24-2.48 (m, 5H) 1.65 (br dd, J=13.63, 1.88 Hz, 1H) 1.21 (s, 2H) 0.97-1.12 (m, 2H).

### Synthesis of Compound 1509

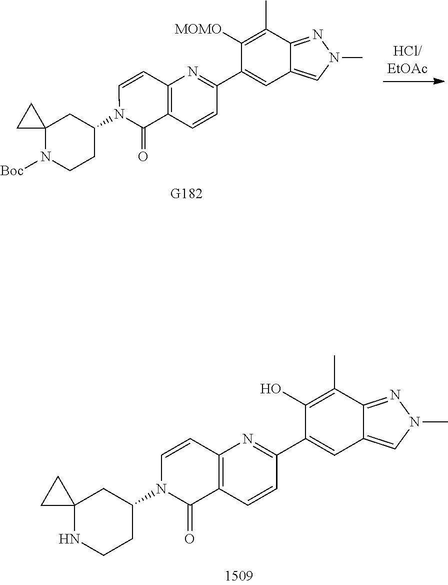

A solution of tert-butyl (7R)-7-[2-[6-(methoxymethoxy)-2,7-dimethyl-indazol-5-yl]-5-oxo-1,6-naphthyridin-6-yl]-4-azaspiro[2.5]octane-4-carboxylate (168-1p2, 100 mg, 179 umol, 1.00 eq) in HCl (gas)/ethyl acetate (2.0 M, 20.0 mL) was stirred at 20° C. for 2 h, then concentrated to give a residue which was triturated with dichloromethane (10.0 mL) to form a suspension. The resulting suspension was filtered and the filter cake was dried to give 6-[(7R)-4-azaspiro[2.5]octan-7-yl]-2-(6-hydroxy-2,7-dimethyl-indazol-5-yl)-1,6-naphthyridin-5-one (72.5 mg, 90%) as a solid. LCMS: (ESI, m/z): 416.3 [M+H]⁺. ¹H NMR (400 MHz, METHANOL-d₄) δ ppm 8.62 (d, J=8.76 Hz, 1H) 8.47 (d, J=10.13 Hz, 2H) 8.20 (d, J=8.88 Hz, 1H) 7.80 (d, J=7.88 Hz, 1H) 6.87 (d, J=7.63 Hz, 1H) 5.14-5.31 (m, 1H) 4.25 (s, 3H) 3.57-3.71 (m, 1H) 3.40 (td, J=12.82, 3.25 Hz, 1H) 2.79 (t, J=12.94 Hz, 1H) 2.24-2.48 (m, 5H) 1.65 (br dd, J=13.76, 1.88 Hz, 1H) 1.15-1.26 (m, 2H) 0.97-1.08 (m, 2H).

### Example G96: Synthesis of Compound 1519

### Synthesis of Intermediate G183

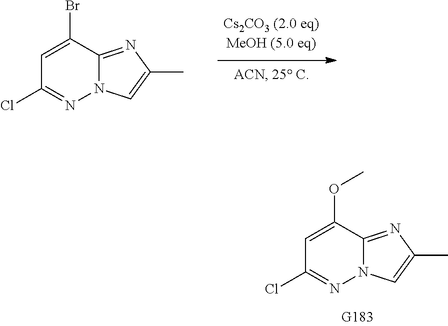

To a mixture of 8-bromo-6-chloro-2-methyl-imidazo[1,2-b]pyridazine (3.00 g, 12.2 mmol, 1.00 eq) and methanol (1.95 g, 60.9 mmol, 2.46 mL, 5.00 eq) in acetonitrile (60.0 mL) was added cesium carbonate (7.93 g, 24.3 mmol, 2.00 eq). The reaction mixture was stirred at 25° C. for 6 h, then diluted with water (100 mL) and extracted with ethyl acetate (3×60.0 mL). The organic layers were combined, washed with brine (100 mL), dried over Na₂SO₄, filtered, and the filtrate concentrated under reduced pressure to give a residue. The residue was purified by column chromatography on silica gel, eluting with 35% ethyl acetate in petroleum ether to give 6-chloro-8-methoxy-2-methyl-imidazo[1,2-b]pyridazine (2.20 g, 92%) as a solid. LCMS: (ESI, m/z): 198.6 [M+H]⁺. ¹H NMR (400 MHz, CDCl₃) δ ppm 7.60 (s, 1H) 6.36 (s, 1H) 4.08 (s, 3H) 2.45 (s, 3H).

### Synthesis of Intermediate G184

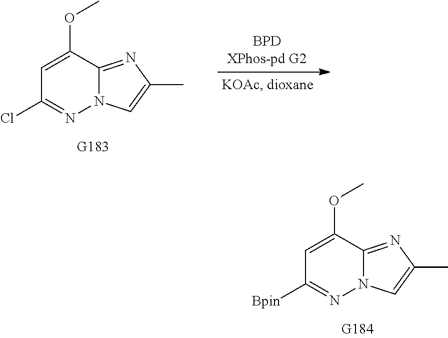

To a solution of 6-chloro-8-methoxy-2-methyl-imidazo[1,2-b]pyridazine (50.0 mg, 253 umol, 1.00 eq) in 1,4-dioxane (1.00 mL) was added 4,4,5,5-tetramethyl-2-(4,4,5,5-tetramethyl-1,3,2-dioxaborolan-2-yl)-1,3,2-dioxaborolane (192 mg, 759 umol, 3.00 eq), potassium acetate (49.6 mg, 506 umol, 2.00 eq), and chloro(2-dicyclohexylphosphino-2′,4′,6′-triisopropyl-1,1′-biphenyl)[2-(2′-amino-1,1′-biphenyl)]palladium (II)(19.9 mg, 25.3 umol, 0.10 eq) at 25° C. under N₂ atmosphere. The reaction mixture was stirred at 100° C. for 1 h under N₂ atmosphere. Nineteen additional reaction mixtures were set up in parallel and the twenty reaction mixtures were combined. The combined reaction mixtures were filtered, and the organic layer was concentrated under reduced pressure to give 8-methoxy-2-methyl-6-(4,4,5,5-tetramethyl-1,3,2-dioxaborolan-2-yl)imidazo[1,2-b]pyridazine (2.80 g, 98%) as an oil.

### Synthesis of Intermediate G185

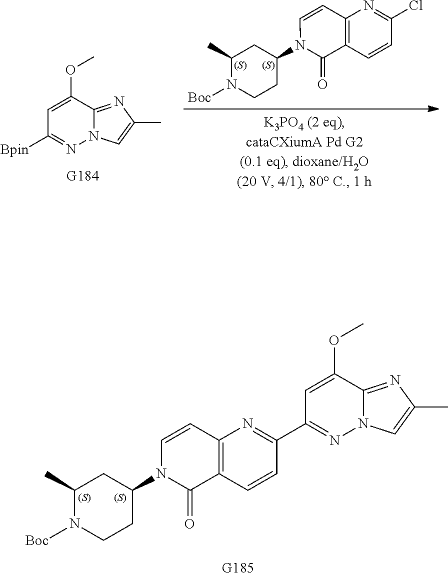

To a mixture of tert-butyl (2S,4S)-4-(2-chloro-5-oxo-1,6-naphthyridin-6-yl)-2-methyl-piperidine-1-carboxylate (40.0 mg, 105 umol, 1.00 eq) and 8-methoxy-2-methyl-6-(4,4,5,5-tetramethyl-1,3,2-dioxaborolan-2-yl)imidazo[1,2-b]pyridazine (183 mg, 635 umol, 6.00 eq) in 1,4-dioxane (640 uL) and water (160 uL) was added K₃PO₄ (44.9 mg, 211 umol, 2.00 eq) and [2-(2-aminophenyl)phenyl]-chloro-palladium; bis(1-adamantyl)-butyl-phosphane (7.08 mg, 10.59 umol, 0.10 eq) at 25° C. The reaction mixture was stirred at 80° C. for 1 h under N₂ atmosphere. Four additional reaction mixtures were set up in parallel and the five reaction mixtures were combined. The combined reaction mixtures were poured into water (20.0 mL) and extracted with ethyl acetate (3×20.0 mL). The organic layers were combined, washed with brine (50.0 mL), dried with anhydrous Na₂SO₄, filtered, and the filtrate concentrated under reduced pressure to give a residue. The residue was purified by silica gel column chromatography(petroleum ether/ethyl acetate=1/1) to give tert-butyl (2S,4S)-4-[2-(8-methoxy-2-methyl-imidazo[1,2-b]pyridazin-6-yl)-5-oxo-1,6-naphthyridin-6-yl]-2-methyl-piperidine-1-carboxylate (150 mg, 56%) as a solid. LCMS: (ESI, m/z): 506.5 [M+H]⁺. ¹H NMR (400 MHz, METHANOL-d₄) δ ppm 8.72 (d, J=8.50 Hz, 1H) 8.44 (d, J=8.50 Hz, 1H) 7.92 (s, 1H) 7.73-7.82 (m, 2H) 6.98 (d, J=7.88 Hz, 1H) 4.96-5.04 (m, 1H) 4.23 (s, 3H) 4.02-4.08 (m, 1H) 3.82-3.91 (m, 1H) 3.44-3.51 (m, 1H) 2.46 (s, 3H) 2.24-2.37 (m, 1H) 2.12-2.22 (m, 1H) 1.95-2.01 (m, 1H) 1.81 (br d, J=2.38 Hz, 1H) 1.52 (s, 9H) 1.31 (d, J=6.38 Hz, 3H).

### Synthesis of Compound 1519

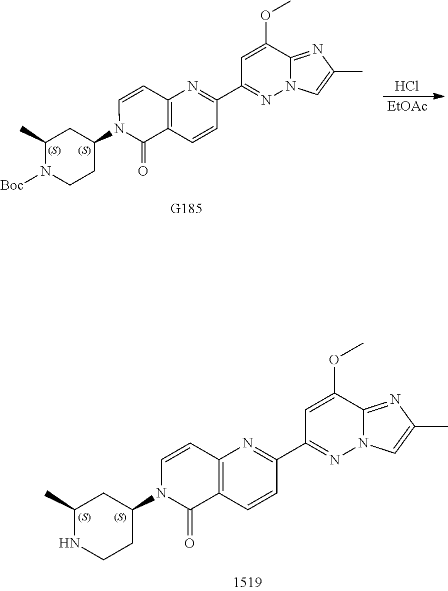

To a solution of tert-butyl (2S,4S)-4-[2-(8-methoxy-2-methyl-imidazo[1,2-b]pyridazin-6-yl)-5-oxo-1,6-naphthyridin-6-yl]-2-methyl-piperidine-1-carboxylate (150 mg, 297 umol, 1.00 eq) in ethyl acetate (3.00 mL) was added HCl/ethyl acetate (4.0 M, 3.00 mL) at 25° C. The reaction mixture was stirred at 25° C. for 4 h, then concentrated under reduced pressure to give a residue. The residue was triturated with methyl tert-butyl ether (5.00 mL) to give a suspension. The resulting suspension was filtered and the filter cake was concentrated under reduced pressure to give 2-(8-methoxy-2-methyl-imidazo[1,2-b]pyridazin-6-yl)-6-[(2S,4S)-2-methyl-4-piperidyl]-1,6-naphthyridin-5-one (101 mg, 77%) as a solid. LCMS: (ESI, m/z): 405.3 [M+H]⁺. ¹H NMR (400 MHz, METHANOL-d₄) δ ppm 8.75 (d, J=8.50 Hz, 1H) 8.47 (d, J=8.50 Hz, 1H) 7.93 (s, 1H) 7.76 (d, J=7.88 Hz, 2H) 7.00 (d, J=7.63 Hz, 1H) 5.07 (br s, 1H) 4.24 (s, 3H) 3.26 (br s, 1H) 2.88-2.98 (m, 2H) 2.47 (s, 3H) 1.89-1.99 (m, 3H) 1.58-1.71 (m, 1H) 1.23 (d, J=6.13 Hz, 3H).

### Example G97: Synthesis of Compound 1520

### Synthesis Intermediate G186

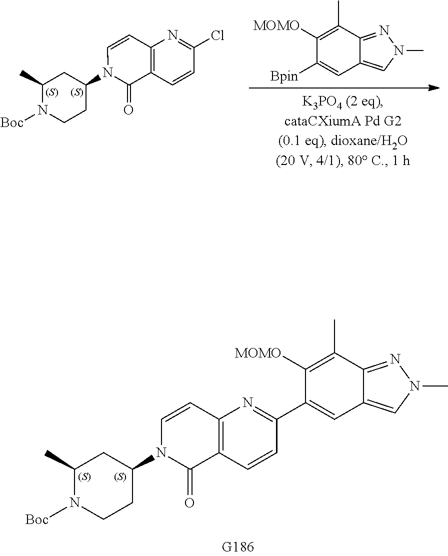

To a mixture of tert-butyl (2S,4S)-4-(2-chloro-5-oxo-1,6-naphthyridin-6-yl)-2-methyl-piperidine-1-carboxylate (40.0 mg, 105 umol, 1.00 eq) and 6-(methoxymethoxy)-2,7-dimethyl-5-(4,4,5,5-tetramethyl-1,3,2-dioxaborolan-2-yl) indazole (52.7 mg, 158 umol, 1.50 eq) in 1,4-dioxane (640 uL) and water (160 uL) was added [2-(2-aminophenyl)phenyl]-chloro-palladium; bis(1-adamantyl)-butyl-phosphane (7.08 mg, 10.59 umol, 0.10 eq) and K₃PO₄ (44.9 mg, 211 umol, 2.00 eq) at 25° C. The reaction mixture was stirred at 80° C. for 1 h under N₂ atmosphere. Four additional reaction mixtures were set up in parallel and the five reaction mixtures were combined and cooled to room temperature. The combined reaction mixture was poured into water (30.0 mL) and extracted with ethyl acetate (3×30.0 mL). The organic layers were combined, washed with brine (90.0 mL), dried with anhydrous Na₂SO₄, filtered, and the filtrate concentrated to give a residue. The residue was purified by silica gel column chromatography (petroleum ether/ethyl acetate=1/1), followed by prep-HPLC (Condition G1, Gradient 6) to give tert-butyl (2S,4S)-4-[2-[6-(methoxymethoxy)-2,7-dimethyl-indazol-5-yl]-5-oxo-1,6-naphthyridin-6-yl]-2-methyl-piperidine-1-carboxylate (140 mg, 48%) as a solid. LCMS: (ES, m/z): 548.5 [M+H]^{+ 1}H NMR (400 MHz, METHANOL-d₄) δ ppm 8.66 (d, J=8.38 Hz, 1H) 8.29 (s, 1H) 7.83-7.88 (m, 2H) 7.78 (d, J=7.75 Hz, 1H) 6.93 (d, J=7.75 Hz, 1H) 5.01 (br d, J=9.26 Hz, 1H) 4.72 (s, 2H) 4.24 (s, 3H) 4.02-4.11 (m, 1H) 3.86 (ddd, J=13.88, 7.19, 2.44 Hz, 1H) 3.47 (ddd, J=14.04, 10.22, 6.13 Hz, 1H) 3.13 (s, 3H) 2.59 (s, 3H) 2.26-2.38 (m, 1H) 2.12-2.22 (m, 1H) 1.93-2.01 (m, 1H) 1.77-1.86 (m, 1H) 1.51 (s, 9H) 1.31 (d, J=6.38 Hz, 3H).

### Synthesis of Compound 1520

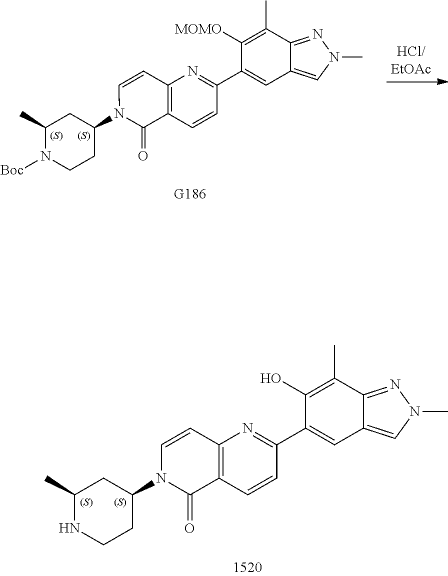

To a solution of tert-butyl (2S,4S)-4-[2-[6-(methoxymethoxy)-2,7-dimethyl-indazol-5-yl]-5-oxo-1,6-naphthyridin-6-yl]-2-methyl-piperidine-1-carboxylate (72-1, 140 mg, 255 umol, 1.00 eq) in ethyl acetate (2.80 mL) was added HCl/ethyl acetate (4.0 M, 2.80 mL) at 25° C. The reaction mixture was stirred at 25° C. for 4 h, then concentrated under reduced pressure to give a residue. The residue was triturated with methyl tert-butyl ether (5.00 mL) to give a suspension. The resulting suspension was filtered and the filter cake was concentrated under reduced pressure to give 2-(6-hydroxy-2,7-dimethyl-indazol-5-yl)-6-[(2S,4S)-2-methyl-4-piperidyl]-1,6-naphthyridin-5-one (97.1 mg, 94%) as a solid. LCMS: (ESI, m/z): 404.2 [M+H]⁺. ¹H NMR (400 MHz, METHANOL-d₄) δ ppm 8.83 (d, J=8.76 Hz, 1H) 8.73 (s, 1H) 8.67 (s, 1H) 8.37 (d, J=8.88 Hz, 1H) 7.77 (d, J=7.75 Hz, 1H) 6.95 (d, J=7.75 Hz, 1H) 5.09-5.19 (m, 1H) 4.33 (s, 3H) 3.59-3.68 (m, 1H) 3.48-3.56 (m, 1H) 3.33 (br s, 1H) 2.48 (s, 3H) 2.27-2.35 (m, 1H) 2.18-2.27 (m, 2H) 2.06-2.18 (m, 1H) 1.44 (d, J=6.50 Hz, 3H).

### Example G98: Synthesis of Compound 1439 and 1440

### Synthesis of Intermediate G187

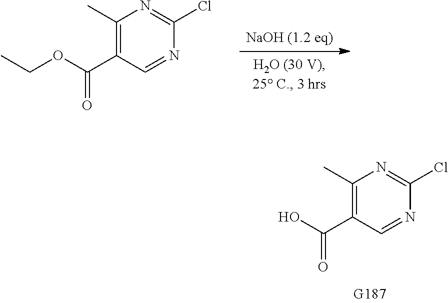

To a solution of ethyl 2-chloro-4-methyl-pyrimidine-5-carboxylate (1.50 g, 7.48 mmol, 1.00 eq) in water (45.0 mL) was added NaOH (359 mg, 8.97 mmol, 1.20 eq) at 25° C. The reaction mixture was stirred at 25° C. for 3 h, then acidified to pH 2 with HCl (6N). A precipitate formed that was collected by filtration and washed with water (3×5.00 mL), then dried to give 2-chloro-4-methyl-pyrimidine-5-carboxylic acid (800 mg, 62%) as a solid. LCMS: (ESI, m/z): 173.0 [M+H]⁺. 1H NMR (400 MHz, DMSO-d₆) δ ppm 13.75-14.00 (m, 1H) 9.01 (s, 1H) 2.73 (s, 3H).

### Synthesis of Intermediate G188

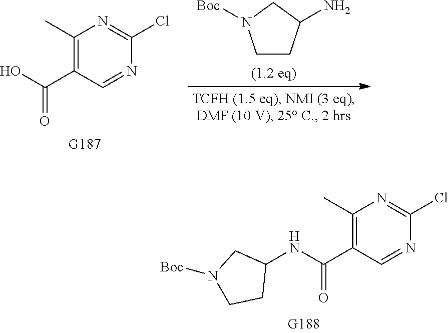 [SMILES: Cc1nc(Cl)ncc1C(=O)O ; CC(C)(C)OC(=O)N1CCC(N)C1 ; Cc1nc(Cl)ncc1C(=O)NC1CCN(C(=O)OC(C)(C)C)C1]

To a solution of 2-chloro-4-methyl-pyrimidine-5-carboxylic acid (800 mg, 4.64 mmol, 1.00 eq) in dimethyl formamide (8.00 mL) was added [chloro(dimethylamino)methylene]-dimethyl-ammonium; hexafluorophosphate (1.95 g, 6.95 mmol, 1.50 eq), 1-methylimidazole (1.14 g, 13.9 mmol, 1.11 mL, 3.00 eq), and tert-butyl 3-aminopyrrolidine-1-carboxylate (1.04 g, 5.56 mmol, 1.20 eq) at 25° C. under N₂ atmosphere. The reaction mixture was stirred at 25° C. for 2 h, then poured into water (40.0 mL) and extracted with ethyl acetate (3×40.0 mL). The organic layers were combined, washed with brine (100 mL), dried with anhydrous Na₂SO₄, filtered, and the filtrate concentrated under reduced pressure to give a residue. The residue was purified by silica gel chromatography (petroleum ether/ethyl acetate=1/1) to give tert-butyl 3-[(2-chloro-4-methyl-pyrimidine-5-carbonyl)amino]pyrrolidine-1-carboxylate (1.30 g, 82%) as a solid. LCMS: (ESI, m/z): 285.1 [M−56+H]⁺. ¹H NMR (400 MHz, DMSO-d₆) δ ppm 8.85 (br d, J=6.48 Hz, 1H) 8.69 (s, 1H) 4.33-4.40 (m, 1H) 3.46-3.55 (m, 1H) 3.30-3.37 (m, 2H) 3.16-3.23 (m, 1H) 2.67 (s, 3H) 2.46-2.49 (m, 2H) 1.38 (s, 9H).

### Synthesis of Intermediate G189

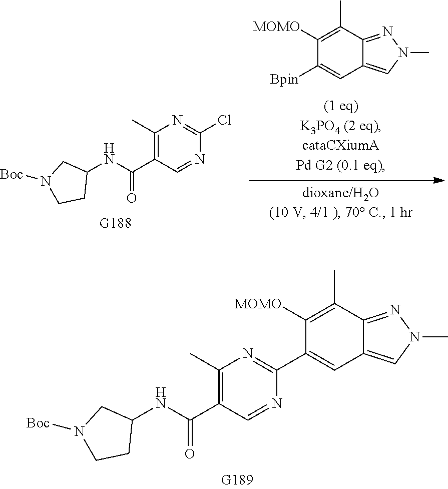

To a mixture of tert-butyl 3-[(2-chloro-4-methyl-pyrimidine-5-carbonyl)amino]pyrrolidine-1-carboxylate (36-2, 400 mg, 1.17 mmol, 1.00 eq) in 1,4-dioxane (3.20 mL) and water (0.80 mL) was added 6-(methoxymethoxy)-2,7-dimethyl-5-(4,4,5,5-tetramethyl-1,3,2-dioxaborolan-2-yl)indazole (389 mg, 1.17 mmol, 1.00 eq), K₃PO₄ (498 mg, 2.35 mmol, 2.00 eq), and [2-(2-aminophenyl)phenyl]-chloro-palladium; bis(1-adamantyl)-butyl-phosphane (78.4 mg, 117 umol, 0.10 eq) at 25° C. under N₂ atmosphere. The reaction mixture was stirred at 70° C. for 1 h. One additional reaction mixture was set up in parallel and the two reaction mixtures were combined and cooled to room temperature. The combined reaction mixture was poured into water (20.0 mL) and extracted with ethyl acetate (3×20.0 mL). The organic layers were combined, washed with brine (50.0 mL), dried with anhydrous Na₂SO₄, filtered, and the filtrate concentrated to give a residue. The residue was purified by silica gel column chromatography (ethyl acetate/methanol=4/1) to give tert-butyl 3-[[2-[6-(methoxymethoxy)-2,7-dimethyl-indazol-5-yl]-4-methyl-pyrimidine-5-carbonyl]amino]pyrrolidine-1-carboxylate (900 mg, 75%) as a solid. LCMS: (ESI, m/z): 511.2 [M+H]⁺. ¹H NMR (400 MHz, DMSO-d₆) δ ppm 8.88 (br d, J=6.11 Hz, 1H) 8.78 (s, 1H) 8.41 (s, 1H) 7.91 (s, 1H) 4.86 (s, 2H) 4.42 (br s, 1H) 4.18 (s, 3H) 3.52-3.59 (m, 1H) 3.36-3.44 (m, 2H) 3.32 (s, 3H) 3.27 (s, 3H) 2.57 (s, 3H) 2.43-2.47 (m, 1H) 2.08-2.15 (m, 1H) 1.86-1.92 (m, 1H) 1.41 (s, 9H).

### Synthesis of Intermediate G190

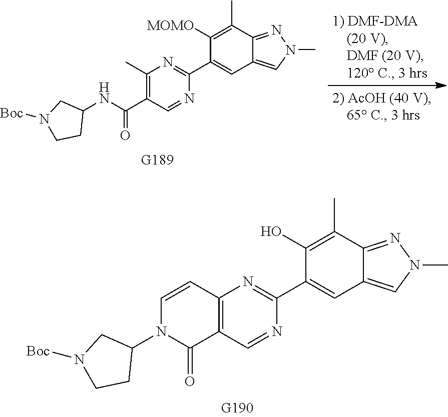

To a solution of tert-butyl 3-[[2-[6-(methoxymethoxy)-2,7-dimethyl-indazol-5-yl]-4-methyl-pyrimidine-5-carbonyl]amino]pyrrolidine-1-carboxylate (200 mg, 391 umol, 1.00 eq) in dimethyl formamide (4.00 mL) was added 1,1-dimethoxy-N,N-dimethylmethanamine (4.00 mL) at 25° C. under N₂ atmosphere. The reaction mixture was stirred at 120° C. for 3 h, then concentrated under reduced pressure to give a residue. To the residue was added acetic acid (8.00 mL) and the resulting mixture was stirred at 65° C. for 6 h. The reaction mixture was concentrated under reduced pressure to give a residue, which was triturated with methyl tert-butyl ether (5.00 mL) to give a suspension. The suspension was filtered to collect tert-butyl 3-[2-(6-hydroxy-2,7-dimethyl-indazol-5-yl)-5-oxo-pyrido[4,3-d]pyrimidin-6-yl]pyrrolidine-1-carboxylate (100 mg, 54%) as a solid. LCMS: (ESI, m/z): 477.2 [M+H]⁺. ¹H NMR (400 MHz, DMSO-d₆) δ ppm 13.12 (s, 1H) 9.55 (s, 1H) 8.97 (s, 1H) 8.47 (s, 1H) 8.01 (br s, 1H) 6.82 (d, J=7.70 Hz, 1H) 5.32-5.38 (m, 1H) 4.15 (s, 2H) 4.11-4.20 (m, 1H) 3.72-3.77 (m, 1H) 3.53 (td, J=5.47, 2.26 Hz, 1H) 3.39-3.43 (m, 2H) 2.37 (s, 3H) 2.23-2.33 (m, 2H) 1.42 (br s, 9H).

### Synthesis of Intermediate G191

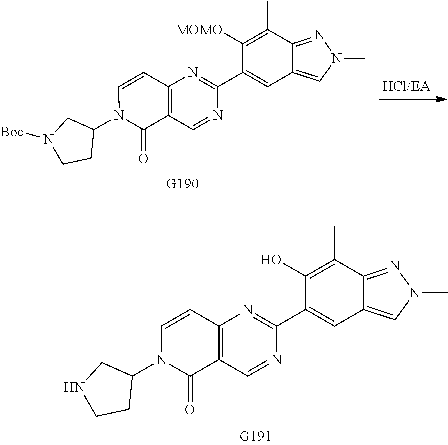

To a solution of tert-butyl 3-[2-(6-hydroxy-2,7-dimethyl-indazol-5-yl)-5-oxo-pyrido[4,3-d]pyrimidin-6-yl]pyrrolidine-1-carboxylate (300 mg, 629 umol, 1.00 eq) in ethyl acetate (6.00 mL) was added HCl/ethyl acetate (4.0 M, 6.00 mL) at 25° C. The reaction mixture was stirred at 25° C. for 3 h, then concentrated under reduced pressure to give 2-(6-hydroxy-2,7-dimethyl-indazol-5-yl)-6-pyrrolidin-3-yl-pyrido[4,3-d]pyrimidin-5-one (160 mg, 89%) as a solid. LCMS: (ESI, m/z): 377.1 [M+H]⁺. ¹H NMR (400 MHz, DMSO-d₆) δ ppm 9.57 (s, 1H) 9.17-9.32 (m, 2H) 8.98 (s, 1H) 8.49 (s, 1H) 8.18 (d, J=7.70 Hz, 1H) 6.89 (d, J=7.58 Hz, 1H) 5.33 (br t, J=6.42 Hz, 1H) 4.15 (s, 3H) 3.57-3.62 (m, 2H) 3.50-3.54 (m, 1H) 3.25-3.31 (m, 1H) 2.45 (br d, J=5.14 Hz, 1H) 2.38 (s, 3H) 2.28-2.34 (m, 1H).

### Synthesis of Compound 1439 and 1440

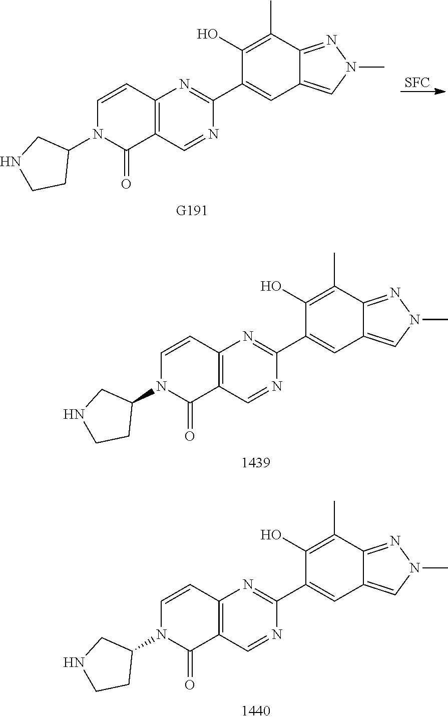

To a solution of 2-(6-hydroxy-2,7-dimethyl-indazol-5-yl)-6-pyrrolidin-3-yl-pyrido[4,3-d]pyrimidin-5-one (150 mg) in methanol (5.00 mL) was added alkaline resin (300 mg). The reaction mixture was stirred at 25° C. for 1 h, then filtered and the filtrate was concentrated to give a residue. The residue was purified by SFC (Condition G3) to give (6R)-2-(6-hydroxy-2,7-dimethyl-indazol-5-yl)-6-pyrrolidin-3-yl-pyrido[4,3-d]pyrimidin-5-one (79.14 mg, 53%) and (6S)-2-(6-hydroxy-2,7-dimethyl-indazol-5-yl)-6-pyrrolidin-3-yl-pyrido[4,3-d]pyrimidin-5-one (42.97 mg, 29%) as solids. 1439: LCMS: (ESI, m/z): 377.1 [M+H]^{+ 1}H NMR (400 MHz, DMSO-d₆) δ ppm 13.14 (s, 1H) 9.55 (s, 1H) 8.97 (s, 1H) 8.47 (s, 1H) 8.15 (d, J=7.70 Hz, 1H) 6.85 (d, J=7.58 Hz, 1H) 5.24-5.39 (m, 1H) 4.15 (s, 3H) 3.27 (br s, 2H) 3.14 (br dd, J=12.53, 4.10 Hz, 1H) 2.97-3.04 (m, 1H) 2.38 (s, 3H) 2.32-2.36 (m, 1H) 1.95-2.03 (m, 1H). 1440: LCMS: (ESI, m/z): 377.1 [M+H]^{+ 1}H NMR (400 MHz, DMSO-d₆) δ ppm 9.56 (s, 1H) 9.19-9.43 (m, 2H) 8.98 (s, 1H) 8.49 (s, 1H) 8.19 (d, J=7.70 Hz, 1H) 6.88 (d, J=7.70 Hz, 1H) 5.31-5.37 (m, 1H) 4.15 (s, 3H) 3.59 (br d, J=8.19 Hz, 2H) 3.52 (br dd, J=10.70, 4.34 Hz, 2H) 3.27-3.32 (m, 1H) 2.38 (s, 3H) 2.29-2.34 (m, 1H).

### Example G99: Synthesis of Compound 1447

### Synthesis of Intermediate G192

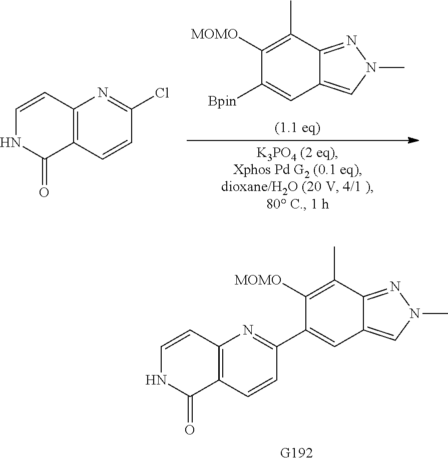

To a mixture of 2-chloro-6H-1,6-naphthyridin-5-one (22-3, 200 mg, 1.11 mmol, 1.00 eq) and 6-(methoxymethoxy)-2,7-dimethyl-5-(4,4,5,5-tetramethyl-1,3,2-dioxaborolan-2-yl)indazole (A22, 404 mg, 1.22 mmol, 1.10 eq) in 1,4-dioxane (3.20 mL) and water (0.80 mL) was added K₃PO₄ (470 mg, 2.21 mmol, 2.00 eq), chloro(2-dicyclohexyl-phosphino-2′,4′,6′-triisopropyl-1,1′-biphenyl)[2-(2′-amino-1,1′-biphenyl)]palladium(II)(87.1 mg, 110 umol, 0.10 eq) at 25° C. under N₂ atmosphere. The reaction mixture was stirred at 80° C. for 1 h, then poured into water (20.0 mL) and extracted with ethyl acetate (3×20.0 mL). The organic layers were combined, washed with brine (50.0 mL), dried with anhydrous Na₂SO₄, filtered, and the filtrate concentrated under reduced pressure to give a residue. The residue was purified by silica gel column chromatography (ethyl acetate) to give 2-[6-(methoxymethoxy)-2,7-dimethyl-indazol-5-yl]-6H-1,6-naphthyridin-5-one (200 mg, 52%) as a solid. LCMS: (ESI, m/z): 351.1 [M+H]⁺. ¹H NMR (400 MHz, DMSO-d₆) δ ppm 8.48 (d, J=8.44 Hz, 1H) 8.41 (s, 1H) 7.88 (s, 1H) 7.78 (d, J=8.31 Hz, 1H) 7.44 (dd, J=7.21, 6.11 Hz, 1H) 6.66 (d, J=7.21 Hz, 1H) 4.69 (s, 2H) 4.18 (s, 3H) 3.11 (s, 3H) 2.49 (br s, 3H).

### Synthesis of Intermediate G193

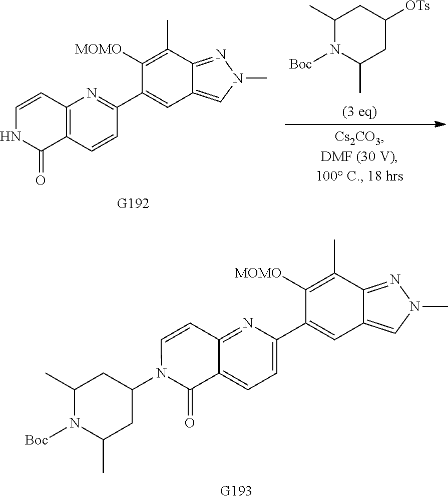

To a solution of 2-[6-(methoxymethoxy)-2,7-dimethyl-indazol-5-yl]-6H-1,6-naphthyridin-5-one (100 mg, 285 umol, 1.00 eq) and tert-butyl 2,6-dimethyl-4-(p-tolylsulfonyloxy)piperidine-1-carboxylate (164 mg, 428 umol, 1.50 eq) in dimethyl formamide (3.00 mL) was added Cs₂CO₃ (278 mg, 856 umol, 3.00 eq) at 25° C. under N₂ atmosphere. The reaction mixture was stirred at 80° C. for 4 hours. Tert-butyl 2,6-dimethyl-4-(p-tolylsulfonyloxy)piperidine-1-carboxylate (164 mg, 428 umol, 1.50 eq) and Cs₂CO₃ (92.9 mg, 285 umol, 1.00 eq) were added to the reaction mixture at 25° C. under N₂ atmosphere. The resulting mixture was stirred at 100° C. for 18 h, then cooled to 25° C., poured into water (20.0 mL), and extracted with ethyl acetate (3×20.0 mL). The organic layers were combined, washed with brine (50.0 mL), dried over anhydrous Na₂SO₄, filtered, and the filtrate concentrated to give a residue. The residue was purified by silica gel column chromatography (ethyl acetate) to give tert-butyl 4-[2-[6-(methoxymethoxy)-2,7-dimethyl-indazol-5-yl]-5-oxo-1,6-naphthyridin-6-yl]-2,6-dimethyl-piperidine-1-carboxylate (35.0 mg, 29%) as a solid. LCMS: (ESI, m/z): 562.3 [M+H]⁺. ¹H NMR (400 MHz, DMSO-d₆) δ ppm 8.54 (dd, J=8.44, 3.79 Hz, 1H) 8.41-8.44 (m, 1H) 7.89 (s, 1H) 7.83 (d, J=2.57 Hz, 1H) 7.81 (d, J=2.32 Hz, 1H) 6.78 (dd, J=7.70, 3.18 Hz, 1H) 5.22-5.54 (m, 1H) 4.69 (s, 2H) 4.42 (br d, J=5.26 Hz, 2H) 4.19 (s, 3H) 3.11-3.15 (m, 3H) 2.49-2.50 (m, 3H) 2.03-2.18 (m, 2H) 1.70-1.78 (m, 2H) 1.44 (s, 9H) 1.25-1.34 (m, 6H).

### Synthesis of Compound 1447

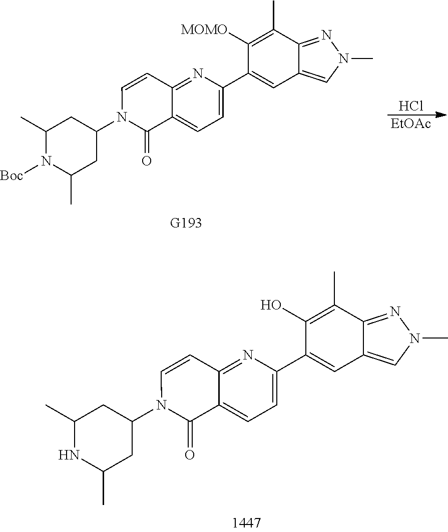

To a solution of tert-butyl 4-[2-[6-(methoxymethoxy)-2,7-dimethyl-indazol-5-yl]-5-oxo-1,6-naphthyridin-6-yl]-2,6-dimethyl-piperidine-1-carboxylate (35.0 mg, 62.3 umol, 1.00 eq) in ethyl acetate (1.00 mL) and was added HCl/ethyl acetate (4.0 M, 1.00 mL) at 25° C. The reaction mixture was stirred at 25° C. for 2 h, then concentrated under reduced pressure to give a residue. The residue was purified by prep-HPLC (Condition G8, Gradient 4) to give 6-(2,6-dimethyl-4-piperidyl)-2-(6-hydroxy-2,7-dimethyl-indazol-5-yl)-1,6-naphthyridin-5-one (4.16 mg, 16%) as a solid. LCMS: (ES, m/z): 418.2 [M+H]⁺. ¹H NMR (400 MHz, DMSO-d₆) δ ppm 8.64 (br d, J=8.68 Hz, 1H) 8.55 (s, 1H) 8.43 (s, 1H) 8.34-8.40 (m, 1H) 7.95 (d, J=7.83 Hz, 1H) 7.70 (d, J=7.46 Hz, 1H) 6.93 (d, J=7.70 Hz, 1H) 6.82 (d, J=7.70 Hz, 1H) 5.17-5.34 (m, 1H) 4.92-5.01 (m, 1H) 4.15 (s, 3H) 3.81-3.96 (m, 1H) 3.44-3.50 (m, 1H) 2.57-2.63 (m, 1H) 2.38 (s, 3H) 2.33 (br s, 1H) 1.88-2.07 (m, 2H) 1.45 (br d, J=7.21 Hz, 1H) 1.27-1.33 (m, 5H).

### Example G100: Synthesis of Compound 1448

### Synthesis of Intermediate G194

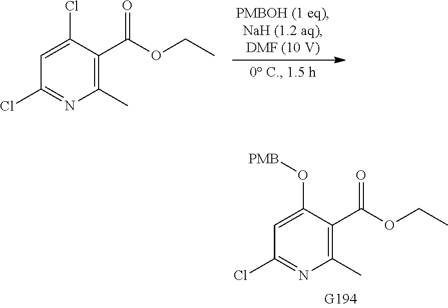

To a mixture of ethyl 4,6-dichloro-2-methyl-pyridine-3-carboxylate (5.00 g, 21.4 mmol, 1 eq) and (4-methoxyphenyl)methanol (2.95 g, 21.4 mmol, 2.66 mL, 1 eq) in N,N-dimethylformamide (50.0 mL) was added sodium hydride (1.03 g, 25.6 mmol, 60%, 1.2 eq) at 0° C. The resulting mixture was stirred at 0° C. for 1.5 h, then quenched with water (200 mL) at 25° C. and extracted with ethyl acetate (6×50.0 mL). The organic layers were combined, dried over Na₂SO₄, filtered, and the filtrate concentrated under reduced pressure to give a residue. The residue was purified by column chromatography (SiO₂, 9% of ethyl acetate in petroleum ether) to give ethyl 6-chloro-4-[(4-methoxyphenyl)methoxy]-2-methyl-pyridine-3-carboxylate (2.50 g, 35%) as a solid. LCMS: (ES, m/z): 336.1 [M+H]⁺. ¹H NMR (400 MHz, CHLOROFORM-d) δ ppm 7.30 (d, J=8.70 Hz, 2H) 6.92 (d, J=8.70 Hz, 2H) 6.79 (s, 1H) 5.07 (s, 2H) 4.36 (q, J=7.07 Hz, 2H) 3.83 (s, 3H) 2.49 (s, 3H) 1.30 (t, J=7.15 Hz, 3H).

### Synthesis of Intermediate G195

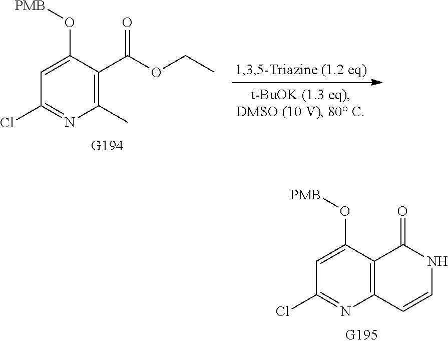

A mixture of ethyl 6-chloro-4-[(4-methoxyphenyl)methoxy]-2-methyl-pyridine-3-carboxylate (0015-2, 2.5 g, 7.45 mmol, 1 eq), potassium tert-butoxide (1.09 g, 9.68 mmol, 1.3 eq), 1,3,5-triazine (724 mg, 8.93 mmol, 1.2 eq) in dimethyl sulfoxide (25.0 mL) was degassed and purged with N₂ 3 times. The reaction mixture was stirred at 80° C. for 4 h under N₂ atmosphere, then cooled to 20° C., quenched with water (200 mL) at 25° C., and extracted with ethyl acetate (6×40.0 mL). The organic layers were combined, dried over Na₂SO₄, filtered, and the filtrate concentrated under reduced pressure to give a residue. The residue was triturated with tert-butyl methyl ether (20.0 mL) to give a suspension. The resulting suspension was filtered to collect the solid, which was dried to give 2-chloro-4-[(4-methoxyphenyl)methoxy]-6H-1,6-naphthyridin-5-one (1.00 g, 42%). LCMS: (ES, m/z): 317.1 [M+H]⁺. ¹H NMR (400 MHz, DMSO-d₆) δ ppm 7.49 (d, J=8.70 Hz, 2H) 7.37-7.46 (m, 1H) 7.17 (s, 1H) 6.98 (d, J=8.70 Hz, 2H) 6.39 (d, J=7.27 Hz, 1H) 5.29 (s, 2H) 3.77 (s, 3H).

### Synthesis of Intermediate G196

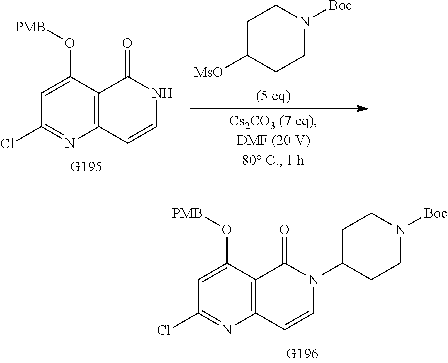

A mixture of 2-chloro-4-[(4-methoxyphenyl)methoxy]-6H-1,6-naphthyridin-5-one (1.00 g, 3.16 mmol, 1 eq), tert-butyl 4-methylsulfonyloxypiperidine-1-carboxylate (4.41 g, 15.8 mmol, 5 eq), and Cs₂CO₃ (7.20 g, 22.1 mmol, 7 eq) in N,N-dimethylformamide (20.0 mL) was degassed and purged with N₂ 3 times. The reaction mixture was stirred at 80° C. for 1 h under N₂ atmosphere, then cooled to 25° C., quenched with water (100 mL), and extracted with ethyl acetate (6×30.0 mL). The organic layers were combined, dried over Na₂SO₄, filtered, and the filtrate concentrated under reduced pressure to give a residue. The residue was purified by column chromatography (SiO₂, 19% of ethyl acetate in petroleum ether) to give tert-butyl 4-[2-chloro-4-[(4-methoxyphenyl)methoxy]-5-oxo-1,6-naphthyridin-6-yl]piperidine-1-carboxylate (300 mg, 19%) as a solid. LCMS: (ES, m/z): 500.2 [M+H]⁺. ¹H NMR (400 MHz, DMSO-d₆) δ ppm 7.84 (d, J=7.70 Hz, 1H) 7.51 (d, J=8.68 Hz, 2H) 7.21 (s, 1H) 6.99 (d, J=8.68 Hz, 2H) 6.47 (d, J=7.70 Hz, 1H) 5.26-5.30 (m, 2H) 4.90 (dq, J=10.38, 5.22 Hz, 1H) 4.02-4.19 (m, 2H) 3.78 (s, 3H) 2.75-2.94 (m, 2H) 1.65-1.82 (m, 4H) 1.42 (s, 9H).

### Synthesis of Intermediate G197

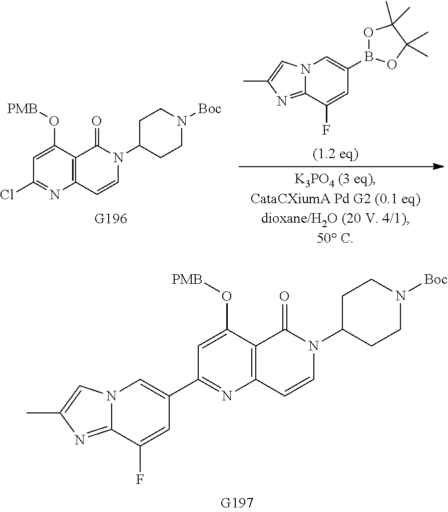

A mixture of 8-fluoro-2-methyl-6-(4,4,5,5-tetramethyl-1,3,2-dioxaborolan-2-yl)-3,8a-dihydroimidazo[1,2-a]pyridine (286 mg, 720 umol, 1.2 eq), tert-butyl 4-[2-chloro-4-[(4-methoxyphenyl)methoxy]-5-oxo-1,6-naphthyridin-6-yl]piperidine-1-carboxylate (300 mg, 600 umol, 1.2 eq), K₃PO₄ (382 mg, 1.08 mmol, 3 eq), and [2-(2-aminophenyl)phenyl]-chloro-palladium; bis(1-adamantyl)-butyl-phosphane (40.1 mg, 36.00 umol, 0.1 eq) in dioxane (4.80 mL) and water (1.20 mL) was degassed and purged with N₂ 3 times. The reaction mixture was stirred at 50° C. for 2 h under N₂ atmosphere, then cooled to 25° C., quenched with water (10.0 mL), and extracted with ethyl acetate (5×10.0 mL). The organic layers were combined, dried over Na₂SO₄, filtered, and the filtrate was concentrated under reduced pressure to give a residue, which was purified by column chromatography on silica gel (SiO₂, 33% of ethyl acetate in petroleum ether) to give tert-butyl 4-[2-(8-fluoro-2-methyl-3,8a-dihydroimidazo[1,2-a]pyridin-6-yl)-4-[(4-methoxyphenyl)methoxy]-5-oxo-1,6-naphthyridin-6-yl]piperidine-1-carboxylate (180 mg, 48%) as a solid. LCMS: (ES, m/z): 614.3 [M+H]⁺. ¹HNMR (400 MHz, DMSO-d₆) δ ppm 9.34 (d, J=1.19 Hz, 1H) 7.94-8.02 (m, 2H) 7.79 (d, J=7.87 Hz, 1H) 7.67 (s, 1H) 7.59 (d, J=8.70 Hz, 2H) 7.02 (d, J=8.58 Hz, 2H) 6.59 (d, J=7.63 Hz, 1H) 5.39 (s, 2H) 4.87-4.99 (m, 1H) 4.08-4.19 (m, 2H) 3.78 (s, 3H) 2.85-2.96 (m, 2H) 2.40 (s, 3H) 1.71-1.79 (m, 4H) 1.43 (s, 9H).

### Synthesis of Compound 1448

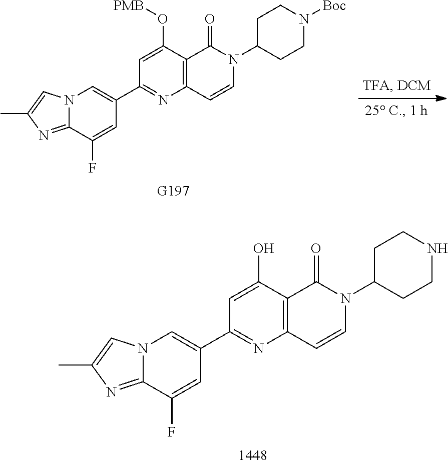

A solution of tert-butyl 4-[2-(8-fluoro-2-methyl-3,8a-dihydroimidazo[1,2-a]pyridin-6-yl)-4-[(4-methoxyphenyl)methoxy]-5-oxo-1,6-naphthyridin-6-yl]piperidine-1-carboxylate (170 mg, 276 umol, 1 eq) in dichloromethane (3.00 mL) was added trifluoroacetic acid (1.75 g, 15.3 mmol, 55.4 eq) dropwise. The reaction mixture was stirred at 25° C. for 1 h, then concentrated in vacuo to give a residue, which was purified by prep-HPLC (Condition G8, Gradient 1) to give tert-butyl 4-[2-(8-fluoro-2-methyl-3,8a-dihydroimidazo[1,2-a]pyridin-6-yl)-4-hydroxy-5-oxo-1,6-naphthyridin-6-yl]piperidine-1-carboxylate (95.0 mg, 61%) as a solid. LCMS: (ES, m/z): 394.2 [M+H]⁺. ¹H NMR (400 MHz, METHANOL-d₄) δ ppm 9.45 (d, J=1.07 Hz, 1H) 8.56 (dd, J=11.21, 1.19 Hz, 1H) 8.19 (dd, J=2.26, 1.19 Hz, 1H) 7.77 (d, J=7.87 Hz, 1H) 7.54 (s, 1H) 6.98 (d, J=7.87 Hz, 1H) 5.09 (tt, J=12.16, 3.93 Hz, 1H) 3.60-3.67 (m, 2H) 3.24-3.30 (m, 2H) 2.62 (d, J=0.95 Hz, 3H) 2.33-2.44 (m, 2H) 2.22 (br d, J=13.47 Hz, 2H). ¹⁹F NMR (376 MHz, METHANOL-d₄) δ ppm−133.936 (s, 1F).

### Example G101: Synthesis of Compound 1453

### Synthesis of Intermediate G198

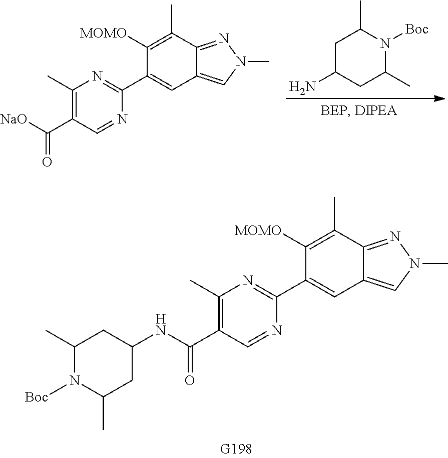

To a mixture of [2-[6-(methoxymethoxy)-2,7-dimethyl-indazol-5-yl]-4-methyl-pyrimidine-5-carbonyl]oxysodium (250 mg, 686.19 umol, 1 eq) and tert-butyl 4-amino-2,6-dimethyl-piperidine-1-carboxylate (156.68 mg, 686.19 umol, 1 eq) in DMF (3 mL) was added 2-bromo-1-ethyl-pyridin-1-ium; tetrafluoroborate (225.51 mg, 823.43 umol, 1.2 eq) and DIPEA (221.71 mg, 1.72 mmol, 298.80 uL, 2.5 eq). The reaction mixture was stirred at 25° C. for 12 h, then quenched with water (1 mL) at 25° C. and concentrated under reduced pressure to give a residue. The residue was purified by prep-HPLC to give tert-butyl 4-[[2-[6-(methoxymethoxy)-2,7-dimethyl-indazol-5-yl]-4-methyl-pyrimidine-5-carbonyl]amino]-2,6-dimethyl-piperidine-1-carboxylate (300 mg, 71%) as a solid. LCMS: (ESI, m/z): 553.4 [M+H]⁺. ¹H NMR (CHLOROFORM-d, 400 MHz) δ 8.7-8.8 (m, 1H), 7.9-8.0 (m, 2H), 7.3-7.3 (m, 1H), 4.9-4.9 (m, 1H), 4.3-4.7 (m, 2H), 4.24 (s, 2H), 3.6-3.9 (m, 1H), 3.4-3.4 (m, 2H), 3.1-3.2 (m, 1H), 2.96 (s, 3H), 2.89 (s, 3H), 2.7-2.8 (m, 2H), 2.66 (s, 2H), 2.0-2.0 (m, 1H), 1.5-1.5 (m, 10H), 1.4-1.4 (m, 3H), 1.3-1.4 (m, 4H).

### Synthesis of Intermediate G199

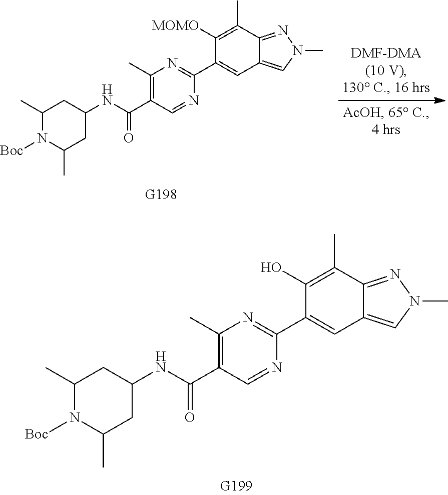

A solution of tert-butyl 4-[[2-[6-(methoxymethoxy)-2,7-dimethyl-indazol-5-yl]-4-methyl-pyrimidine-5-carbonyl]amino]-2,6-dimethyl-piperidine-1-carboxylate (20 mg, 36.19 umol, 1 eq) in DMF-DMA (448.50 mg, 3.76 mmol, 0.5 mL, 104.01 eq) was stirred at 130° C. for 4 h under N₂ atmosphere. The reaction mixture was concentrated under reduced pressure to give a residue. To the residue was added acetic acid (1.05 g, 17.49 mmol, 1 mL, 483.18 eq) and the resulting mixture was stirred at 65° C. for 4 h, then extracted with DCM (3×3 mL). The organic layers were combined, dried over MgSO₄, filtered, and the filtrate concentrated under reduced pressure to give a residue. The residue was purified by prep-HPLC (Condition G1, Gradient 10) to give tert-butyl 4-[2-(6-hydroxy-2,7-dimethyl-indazol-5-yl)-5-oxo-pyrido[4,3-d]pyrimidin-6-yl]-2,6-dimethyl piperidine-1-carboxylate (200 mg, 69%) as a solid. ¹H NMR (DMSO-d₆, 400 MHz) δ 13.13 (s, 1H), 9.5-9.6 (m, 1H), 8.97 (s, 1H), 8.47 (s, 1H), 8.1-8.2 (m, 1H), 6.8-6.8 (m, 1H), 5.1-5.5 (m, 1H), 4.43 (br d, 2H, J=2.6 Hz), 4.15 (s, 3H), 2.38 (s, 3H), 2.11 (dt, 2H, J=6.7, 12.9 Hz), 1.7-1.9 (m, 2H), 1.44 (s, 9H), 1.28 (br d, 6H, J=6.7 Hz).

### Synthesis of Compound 1453

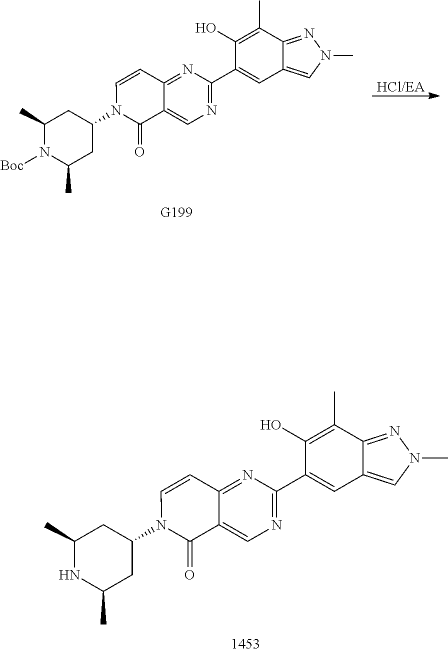

To a solution of tert-butyl (2S,6R)-4-[2-(6-hydroxy-2,7-dimethyl-indazol-5-yl)-5-oxo-pyrido[4,3-d]pyrimidin-6-yl]-2,6-dimethyl-piperidine-1-carboxylate (110 mg, 212.11 umol, 1 eq) in ethyl acetate (2 mL) was added HCl/EtOAc (4 M, 53.03 uL, 1 eq). The reaction mixture was stirred at 25° C. for 6 h under N₂ atmosphere, then concentrated under reduced pressure to give a residue. The residue was purified by re-crystallization from ethyl acetate (10 mL) at 25° C. to give 6-[(2S,6R)-2,6-dimethyl-4-piperidyl]-2-(6-hydroxy-2,7-dimethyl-indazol-5-yl)pyrido[4,3-d]pyrimidin-5-one (90.56 mg, 94%) as a solid. LCMS: (ESI, m/z): 419.2 [M+H]⁺. ¹H NMR (METHANOL-d₄, 400 MHz) δ 9.67 (d, 1H, J=0.6 Hz), 9.26 (s, 1H), 8.82 (s, 1H), 8.14 (d, 1H, J=7.8 Hz), 6.87 (d, 1H, J=7.7 Hz), 5.07 (br t, 1H, J=5.6 Hz), 4.36 (s, 3H), 3.69 (ddd, 2H, J=3.1, 6.2, 12.3 Hz), 2.5-2.6 (m, 2H), 2.49 (s, 3H), 2.10 (ddd, 2H, J=5.7, 12.5, 16.2 Hz), 1.43 (d, 6H, J=6.4 Hz), 1.4-1.4 (m, 1H).

### Example G102: Synthesis of Compound 642

### Synthesis of Compound 642

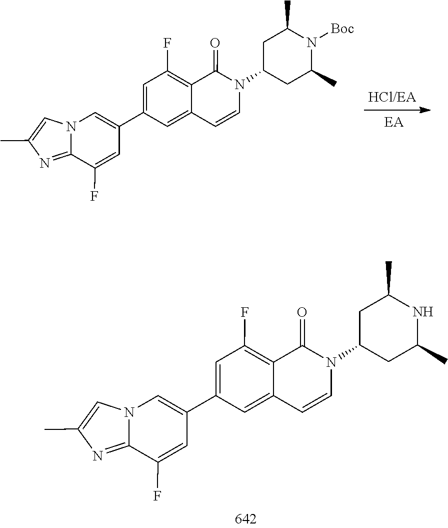

To a solution of tert-butyl (2S,6R)-4-[8-fluoro-6-(8-fluoro-2-methyl-imidazo[1,2-a]pyridin-6-yl)-1-oxo-2-isoquinolyl]-2,6-dimethyl-piperidine-1-carboxylate (20 mg, 38.27 umol, 1 eq) in ethyl acetate (3 mL) was added HCl/EtOAc (4 M, 9.57 uL, 1 eq). The reaction mixture was stirred at 25° C. for 6 h, then concentrated under reduced pressure to give a residue. The residue was purified by re-crystallization from methanol (10.0 mL) at 25° C. to give 2-[(2S,6R)-2,6-dimethyl-4-piperidyl]-8-fluoro-6-(8-fluoro-2-methyl-imidazo[1,2-a]pyridin-6-yl)isoquinolin-1-one (42.70 mg, 60%) as a solid. LCMS: (ES, m/z):423.1 [M+H]⁺. ¹H NMR (METHANOL-d₄, 400 MHz) δ 9.13 (d, 1H, J=1.0 Hz), 8.31 (dd, 1H, J=1.0, 11.1 Hz), 8.15 (d, 1H, J=1.0 Hz), 7.90 (d, 1H, J=1.3 Hz), 7.6-7.7 (m, 2H), 6.85 (dd, 1H, J=1.8, 7.6 Hz), 5.0-5.1 (m, 1H), 3.65 (ddd, 2H, J=3.1, 6.2, 12.2 Hz), 2.63 (d, 3H, J=0.7 Hz), 2.51 (br d, 2H, J=15.9 Hz), 2.06 (ddd, 2H, J=5.7, 12.4, 16.2 Hz), 1.40 (d, 6H, J=6.5 Hz).

### Example G103: Synthesis of Compound 1454

### Synthesis of Compound 1454

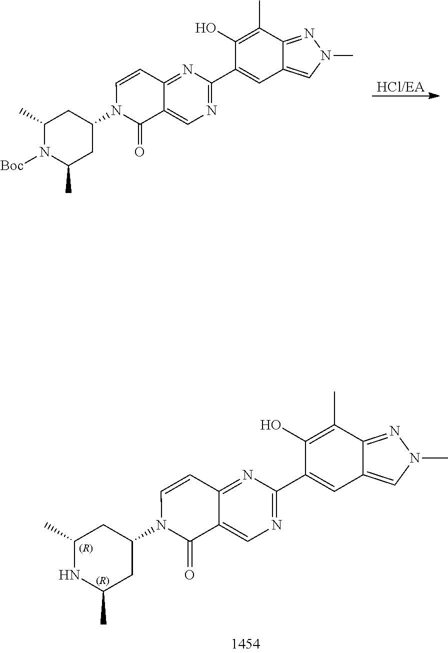

To a solution of tert-butyl (2R,6R)-4-[2-(6-hydroxy-2,7-dimethyl-indazol-5-yl)-5-oxo-pyrido[4,3-d]pyrimidin-6-yl]-2,6-dimethyl-piperidine-1-carboxylate (15 mg, 28.92 umol, 1 eq) in ethyl acetate (2 mL) was added HCl/EtOAc (4 M, 7.23 uL). The reaction mixture was stirred at 25° C. for 6 h, then concentrated under reduced pressure to give a residue. The residue was purified by re-crystallization from ethyl acetate (10 mL) at 25° C. to give 6-[(2R,6R)-2,6-dimethyl-4-piperidyl]-2-(6-hydroxy-2,7-dimethyl-indazol-5-yl)pyrido[4,3-d]pyrimidin-5-one (4.13 mg, 34%) as a solid. LCMS: (ESI, m/z): 419.2 [M+H]⁺. ¹H NMR (METHANOL-d₄, 400 MHz) δ 9.6-9.7 (m, 1H), 9.17 (s, 1H), 8.60 (s, 1H), 7.94 (d, 1H, J=7.9 Hz), 6.85 (d, 1H, J=7.8 Hz), 5.30 (tt, 1H, J=3.6, 12.6 Hz), 4.28 (s, 3H), 4.04 (quin, 1H, J=6.0 Hz), 3.7-3.9 (m, 1H), 2.4-2.5 (m, 3H), 2.38 (dt, 1H, J=5.3, 13.2 Hz), 2.2-2.3 (m, 1H), 2.0-2.1 (m, 2H), 1.60 (d, 3H, J=7.1 Hz), 1.44 (d, 3H, J=6.5 Hz).

### Example G104: Synthesis of Compound 1455

### Synthesis of Compound 1455

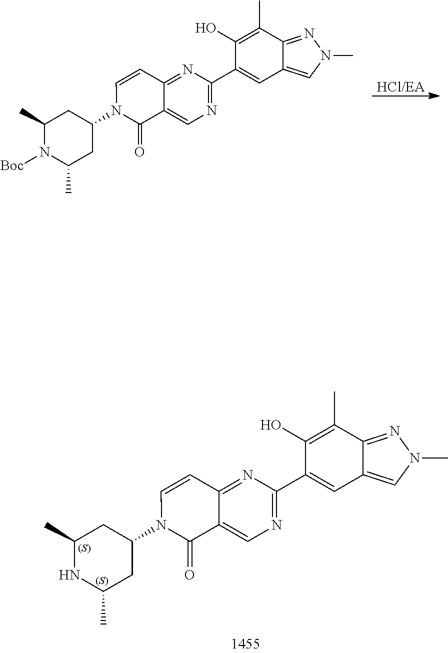

To a solution of tert-butyl (2S,6S)-4-[2-(6-hydroxy-2,7-dimethyl-indazol-5-yl)-5-oxo-pyrido[4,3-d]pyrimidin-6-yl]-2,6-dimethyl-piperidine-1-carboxylate (19.85 mg, 38.27 umol, 1 eq) in ethyl acetate (2 mL) was added HCl/EtOAc (4 M, 9.57 uL, 1 eq). The reaction mixture was stirred at 25° C. for 6 h under N₂ atmosphere, then concentrated under reduced pressure to give a residue. The residue was purified by re-crystallization from ethyl acetate (10 mL) at 25° C. to give 6-[(2S,6S)-2,6-dimethyl-4-piperidyl]-2-(6-hydroxy-2,7-dimethyl-indazol-5-yl)pyrido[4,3-d]pyrimidin-5-one (4.49 mg, 27%) as a solid. LCMS: (ES, m/z):419.2 [M+H]⁺. ¹H NMR (METHANOL-d₄, 400 MHz) δ 9.63 (s, 1H), 9.15 (s, 1H), 8.55 (s, 1H), 7.92 (d, 1H, J=7.8 Hz), 6.84 (d, 1H, J=7.6 Hz), 5.3-5.3 (m, 1H), 4.27 (s, 3H), 4.0-4.1 (m, 1H), 3.79 (ddd, 1H, J=3.3, 6.4, 11.6 Hz), 2.46 (s, 3H), 2.37 (dt, 1H, J=5.1, 13.3 Hz), 2.24 (br d, 1H, J=12.1 Hz), 2.0-2.1 (m, 2H), 1.60 (d, 3H, J=7.1 Hz), 1.44 (d, 3H, J=6.4 Hz).

### Example G105: Synthesis of Compound 1459

### Synthesis of Intermediate G200

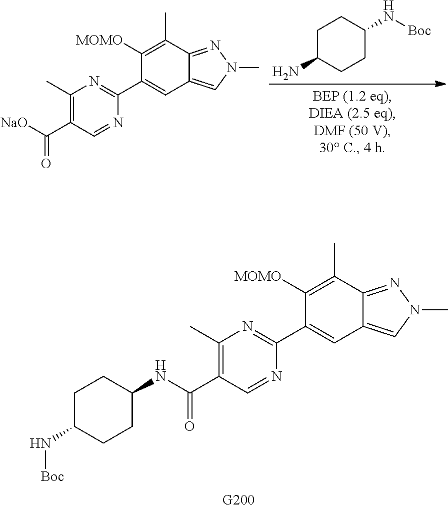

To a mixture of 2-[6-(methoxymethoxy)-2,7-dimethyl-indazol-5-yl]-4-methyl-pyrimidine-5-carboxylic acid (200 mg, 547.44 umol, 1 eq) and tert-butyl ((1r,4r)-4-aminocyclohexyl)carbamate (140.78 mg, 656.93 umol, 1.2 eq) in N,N-dimethylformamide (10 mL) was added 2-bromo-1-ethyl-pyridin-1-ium; tetrafluoroborate (179.91 mg, 656.93 umol, 1.2 eq) and N,N-diisopropylethylamine (176.88 mg, 1.37 mmol, 238.38 uL, 2.5 eq). The reaction mixture was stirred at 30° C. for 2 h, then quenched with water (15 mL) and extracted with dichloromethane (50 mL×4). The organic layers were combined, washed with brine (150 mL), dried over anhydrous sodium sulfate, filtered, and the filtrate concentrated under reduced pressure to give a residue. The residue was purified by silica gel chromatography (petroleum ether/ethyl acetate=35/65) to give tert-butyl ((1r,4r)-4-(4-((E)-2-(dimethylamino)vinyl)-2-(6-(methoxymethoxy)-2,7-dimethyl-2H-indazol-5-yl)pyrimidine-5-carboxamido)cyclohexyl)carbamate (200 mg, 64%) as a solid. LCMS: (ES, m/z): 539.3 [M+H]⁺. ¹HNMR (400 MHz, CDCl₃) δ ppm 8.74 (s, 1H), 7.96 (d, J=15.16 Hz, 2H), 5.75 (br d, J=8.44 Hz, 1H), 4.89 (s, 2H), 4.44 (br dd, J=4.22, 3.48 Hz, 1H), 4.24 (s, 3H), 3.91-4.04 (m, 1H), 3.43-3.56 (m, 1H), 3.40 (s, 3H), 2.74 (s, 3H), 2.66 (s, 3H), 2.09-2.21 (m, 4H), 1.46 (s, 9H), 1.30-1.40 (m, 4H).

### Synthesis of Intermediate G201

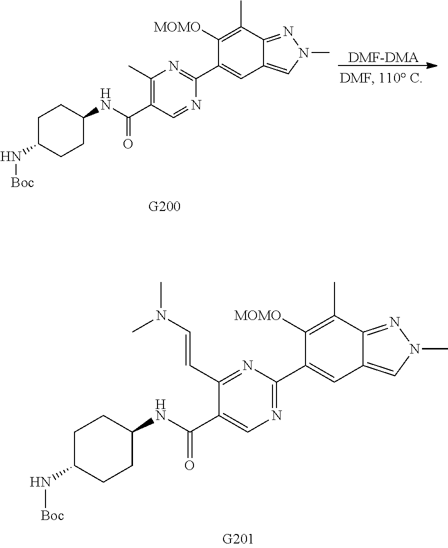

To a solution of tert-butyl N-[4-[[2-[6-(methoxymethoxy)-2,7-dimethyl-indazol-5-yl]-4-methyl-pyrimidine-5-carbonyl]amino]cyclohexyl]carbamate (200 mg, 371.31 umol, 1 eq) in N,N-dimethylformamide (2 mL) was added N,N-dimethylformamide dimethylacetal (2.00 mL). The reaction mixture was stirred at 110° C. for 1 h, then concentrated under reduced pressure to give a residue.

### Synthesis of Intermediate G202

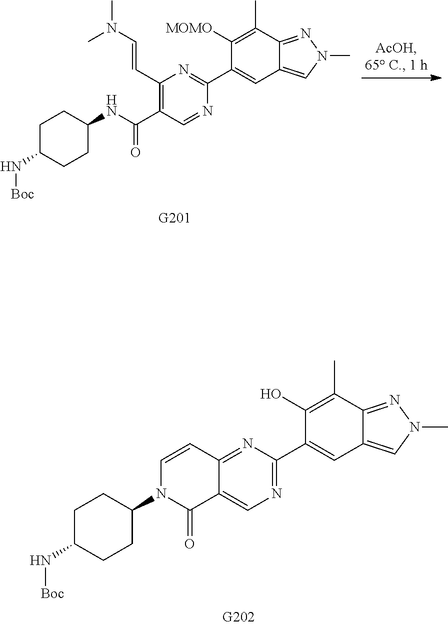

A solution of tert-butyl ((1r, 4r)-4-(4-((E)-2-(dimethylamino) vinyl)-2-(6-(methoxymethoxy)-2,7-dimethyl-2H-indazol-5-yl)pyrimidine-5-carboxamido)cyclohexyl)carbamate (150 mg, 252.65 umol, 1 eq) in acetic acid (1.5 mL) was stirred at 65° C. for 1 h, then filtered. The filter cake was washed with methyl tert-butyl ether (3×1 mL) and dried under vacuum to give tert-butyl ((1r,4r)-4-(2-(6-hydroxy-2,7-dimethyl-2H-indazol-5-yl)-5-oxopyrido[4,3-d]pyrimidin-6(5H)-yl)cyclohexyl)carbamate (80 mg, 63%) as a solid. LCMS: (ES, m/z): 505.2 [M+H]⁺. ¹HNMR (400 MHz, DMSO-d6) δ ppm 13.15 (s, 1H), 9.54 (s, 1H), 8.96 (s, 1H), 8.47 (s, 1H), 8.18 (d, J=7.70 Hz, 1H), 6.74-6.85 (m, 2H), 4.70 (br s, 1H), 4.14 (s, 3H), 3.30 (s, 1H), 2.38 (s, 3H), 1.69-2.03 (m, 8H), 1.40 (s, 9H).

### Synthesis of Intermediate G203

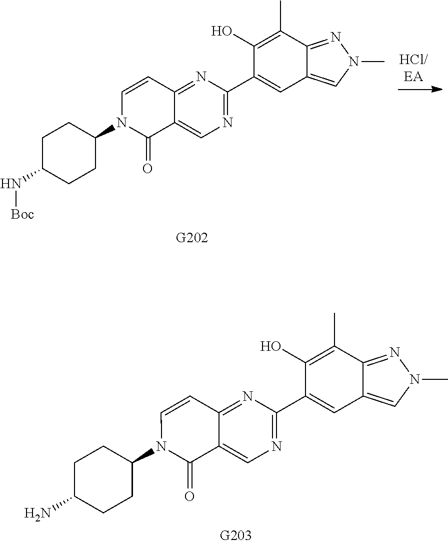 [SMILES: Cc1c(O)c(-c2ncc3c(=O)n([C@H]4CC[C@H](NC(=O)OC(C)(C)C)CC4)ccc3n2)cc2cn(C)nc12 ; Cc1c(O)c(-c2ncc3c(=O)n([C@H]4CC[C@H](N)CC4)ccc3n2)cc2cn(C)nc12]

To a solution of tert-butyl ((1r,4r)-4-(2-(6-hydroxy-2,7-dimethyl-2H-indazol-5-yl)-5-oxopyrido[4,3-d]pyrimidin-6(5H)-yl)cyclohexyl)carbamate (69-7, 90 mg, 178.37 umol, 1 eq) in ethyl acetate (0.9 mL) was added HCl/ethyl acetate (4 M, 0.9 mL, 20.18 eq). The reaction mixture was stirred at 20° C. for 1 h, then concentrated under reduced pressure to give a residue. The residue was washed with dichloromethane (1 mL), dried under vacuum to remove the solvent, then diluted with water (0.5 mL), and lyophilized to give 6-((1r, 4r)-4-aminocyclohexyl)-2-(6-hydroxy-2,7-dimethyl-2H-indazol-5-yl)pyrido[4,3-d]pyrimidin-5(6H)-one (72 mg, 88%) as a solid. LCMS: (ES, m/z): 405.2 [M+H]⁺. ¹HNMR (400 MHz, D₂O) δ ppm 8.41 (s, 1H), 7.59-7.81 (m, 3H), 6.14 (br d, J=7.34 Hz, 1H), 4.37-4.52 (m, 1H), 3.82 (s, 3H), 3.22-3.36 (m, 1H), 2.24 (br d, J=7.46 Hz, 2H), 1.91-2.00 (m, 2H), 1.66-1.84 (m, 4H), 1.65 (s, 3H).

### Synthesis of Compound 1459

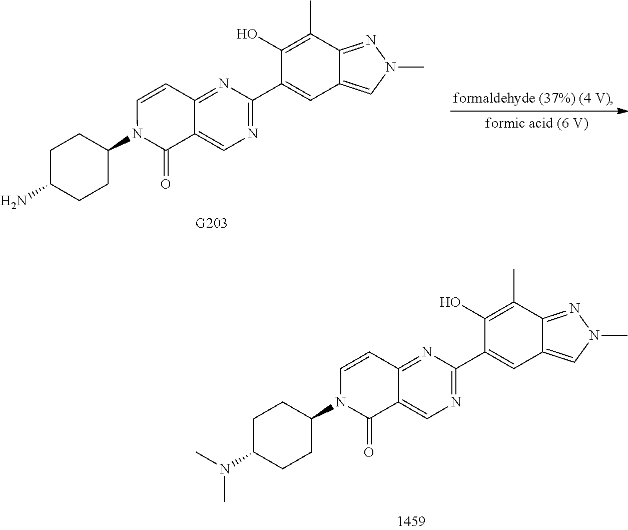

To a solution of 6-(4-aminocyclohexyl)-2-(6-hydroxy-2,7-dimethyl-indazol-5-yl)pyrido[4,3-d]pyrimidin-5-one (72 mg, 163.29 umol, 1 eq, HCl) in formic acid (1.5 mL) was added formaldehyde (1.09 g, 13.43 mmol, 1 mL, 37%, 82.24 eq). The reaction mixture was stirred at 100° C. for 1 h, then poured into a saturated aqueous sodium bicarbonate solution and extracted with ethyl acetate (3×50 mL). The organic layers were combined, washed with a brine, dried over anhydrous sodium sulfate, filtered, and the filtrate concentrated under reduced pressure to give a residue. The residue was purified by prep-HPLC (Condition G8, Gradient 5) to give 6-((1r,4r)-4-(dimethylamino)cyclohexyl)-2-(6-hydroxy-2,7-dimethyl-2H-indazol-5-yl)pyrido[4,3-d]pyrimidin-5(6H)-one (39 mg, 55%) as a solid. LCMS: (ES, m/z): 433.2 [M+H]⁺. ¹HNMR (400 MHz, MeOD) δ ppm 9.66 (s, 1H), 9.23 (s, 1H), 8.76 (s, 1H), 8.03 (d, J=7.78 Hz, 1H), 6.85 (d, J=7.65 Hz, 1H), 4.91 (br s, 1H), 4.33 (s, 3H), 3.40 (s, 1H), 2.93 (s, 6H), 2.47 (s, 3H), 2.30 (br d, J=12.42 Hz, 2H), 2.10-2.19 (m, 2H), 1.94-2.07 (m, 2H), 1.78-1.93 (m, 2H).

### Example G106: Synthesis of Compound 1300

### Synthesis Intermediate G204

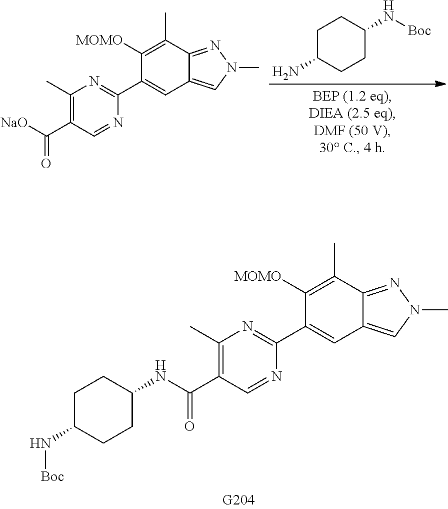

To a mixture of 2-[6-(methoxymethoxy)-2,7-dimethyl-indazol-5-yl]-4-methyl-pyrimidine-5-carboxylic acid (150 mg, 410.58 umol, 1 eq) and tert-butyl ((1s,4s)-4-aminocyclohexyl)carbamate (140.78 mg, 656.93 umol, 1.2 eq) in N,N-dimethylformamide (10 mL) was added 2-bromo-1-ethyl-pyridin-1-ium; tetrafluoroborate (134.93 mg, 492.69 umol, 1.2 eq) and N,N-diisopropylethylamine (132.66 mg, 1.03 mmol, 178.78 uL, 2.5 eq). The reaction mixture was stirred at 30° C. for 2 h, then quenched with water (15 mL) and extracted with dichloromethane (50 mL×4). The organic layers were combined, washed with brine (150 mL), dried over anhydrous sodium sulfate, filtered, and the filtrate concentrated under reduced pressure to give a residue. The residue was purified by silica gel chromatography (petroleum ether/ethyl acetate=35/65) to give tert-butyl ((1s,4s)-4-(2-(6-(methoxymethoxy)-2,7-dimethyl-2H-indazol-5-yl)-4-methylpyrimidine-5-carboxamido)cyclohexyl)carbamate (150 mg, 64%) as a solid. LCMS: (ES, m/z): 539.3 [M+H]⁺. ¹HNMR (400 MHz, CDCl₃) δ ppm 8.75 (s, 1H), 7.97 (d, J=14.92 Hz, 2H), 5.92 (br d, J=7.46 Hz, 1H), 4.90 (s, 2H), 4.51-4.67 (m, 1H), 4.25 (s, 3H), 4.15 (br d, J=6.72 Hz, 1H), 3.70 (br s, 1H), 3.40 (s, 3H), 2.74 (s, 3H), 2.67 (s, 3H), 1.78-1.96 (m, 4H), 1.67 (br s, 4H), 1.46 (s, 9H).

### Synthesis of Intermediate G205

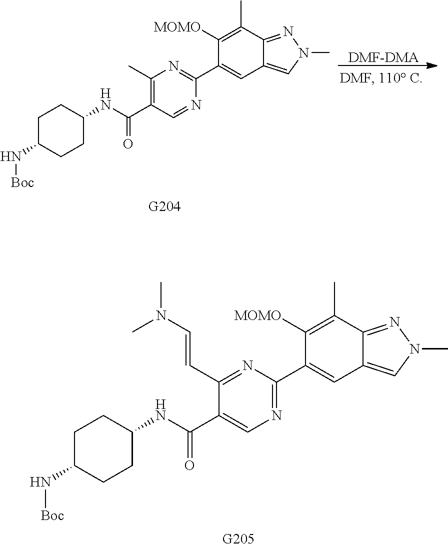

To a solution of tert-butyl ((1s,4s)-4-(2-(6-(methoxymethoxy)-2,7-dimethyl-2H-indazol-5-yl)-4-methylpyrimidine-5-carboxamido)cyclohexyl)carbamate (150 mg, 371.31 umol, 1 eq) in N,N-dimethylformamide (2 mL) was added N,N-dimethylformamide dimethylacetal (2.00 mL). The reaction mixture was stirred at 110° C. for 1 h, then concentrated under reduced pressure to give a residue.

### Synthesis of Intermediate G206

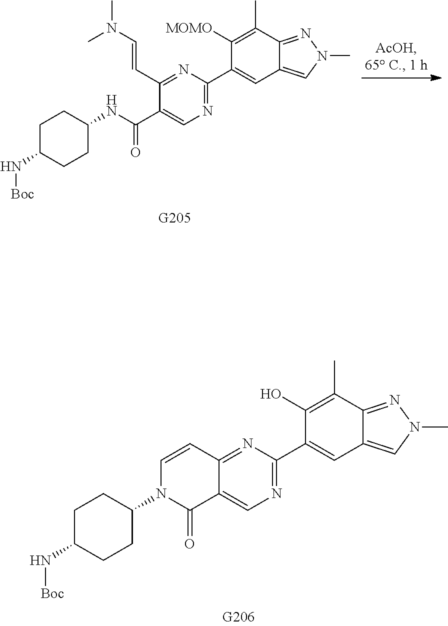

A solution of tert-butyl ((1s,4s)-4-(4-((E)-2-(dimethylamino)vinyl)-2-(6-(methoxymethoxy)-2,7-dimethyl-2H-indazol-5-yl)pyrimidine-5-carboxamido)cyclohexyl)carbamate (150 mg, 252.65 umol, 1 eq) in acetic acid (1.5 mL) was stirred at 65° C. for 1 h, then filtered. The filter cake was washed with methyl tert-butyl ether (3×1 mL) and dried under vacuum to give tert-butyl ((1s,4s)-4-(2-(6-hydroxy-2,7-dimethyl-2H-indazol-5-yl)-5-oxopyrido[4,3-d]pyrimidin-6(5H)-yl)cyclohexyl)carbamate (80 mg, 63%) as a solid. LCMS: (ES, m/z): 505.2 [M+H]⁺. ¹HNMR (400 MHz, DMSO-d6) δ ppm 13.19 (s, 1H), 9.55 (s, 1H), 8.97 (s, 1H), 8.47 (s, 1H), 8.35 (d, J=7.58 Hz, 1H), 7.18 (br d, J=9.17 Hz, 1H), 6.91 (d, J=7.70 Hz, 1H), 4.73-4.88 (m, 1H), 4.15 (s, 3H), 3.79-3.86 (m, 1H), 2.38 (s, 3H), 2.00 (br dd, J=12.47, 4.03 Hz, 2H), 1.55-1.79 (m, 6H), 1.43 (s, 9H).

### Synthesis of Compound 1300

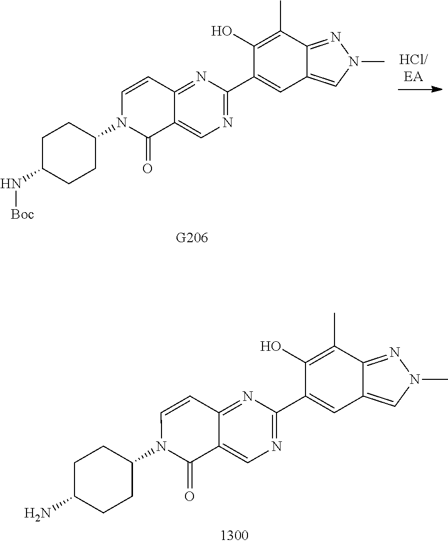 [SMILES: Cc1c(O)c(-c2ncc3c(=O)n([C@H]4CC[C@@H](NC(=O)OC(C)(C)C)CC4)ccc3n2)cc2cn(C)nc12 ; Cc1c(O)c(-c2ncc3c(=O)n([C@H]4CC[C@@H](N)CC4)ccc3n2)cc2cn(C)nc12]

To a solution of tert-butyl ((1s,4s)-4-(2-(6-hydroxy-2,7-dimethyl-2H-indazol-5-yl)-5-oxopyrido[4,3-d]pyrimidin-6(5H)-yl)cyclohexyl)carbamate (90 mg, 178.37 umol, 1 eq) in ethyl acetate (0.9 mL) was added HCl/ethyl acetate (4 M, 0.9 mL, 20.18 eq). The reaction mixture was stirred at 20° C. for 1 h, then concentrated under reduced pressure to give a residue. The residue was washed with dichloromethane (1 mL), dried under vacuum, diluted with water (0.5 mL), and lyophilized to give 6-((1s,4s)-4-aminocyclohexyl)-2-(6-hydroxy-2,7-dimethyl-2H-indazol-5-yl)pyrido[4,3-d]pyrimidin-5(6H)-one (70 mg, 87%) as a solid. LCMS: (ES, m/z): 405.2 [M+H]⁺. ¹HNMR (400 MHz, D₂O) δ ppm 8.49 (s, 1H), 7.84 (s, 1H), 7.70-7.79 (m, 2H), 6.31 (d, J=7.46 Hz, 1H), 4.52 (td, J=10.45, 4.03 Hz, 1H), 3.85 (s, 3H), 3.69 (br s, 1H), 2.08-2.16 (m, 2H), 1.99-2.06 (m, 2H), 1.86 (br d, J=3.91 Hz, 4H), 1.73 (s, 3H).

### Synthesis of Compound 1460

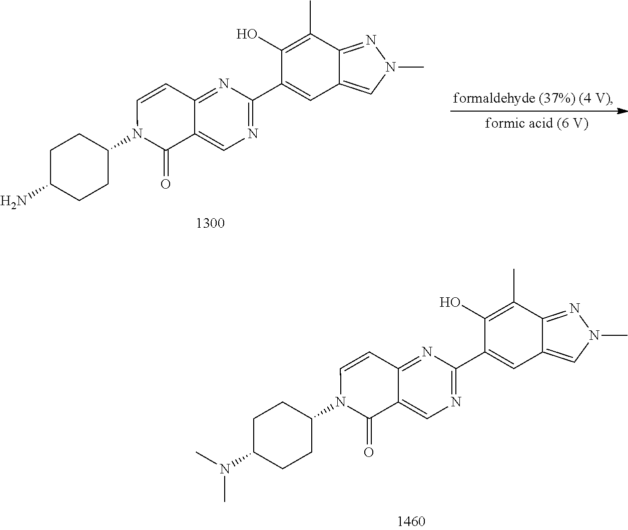

To a solution of 6-((1s,4s)-4-aminocyclohexyl)-2-(6-hydroxy-2,7-dimethyl-2H-indazol-5-yl)pyrido[4,3-d]pyrimidin-5(6H)-one (80 mg, 181.44 umol, 1 eq, HCl) in formic acid (1 mL) was added formaldehyde (1.45 g, 17.91 mmol, 1 mL). The reaction mixture was stirred at 100° C. for 1 h, then poured into a saturated aqueous sodium bicarbonate solution and extracted with ethyl acetate (3×50 mL). The organic layers were combined, washed with brine, dried over anhydrous sodium sulfate, filtered, and the filtrate concentrated under reduced pressure to give a residue. The residue was purified by prep-HPLC (Condition G8, Gradient 5) to give 6-((1s,4s)-4-(dimethylamino)cyclohexyl)-2-(6-hydroxy-2,7-dimethyl-2H-indazol-5-yl)pyrido[4,3-d]pyrimidin-5(6H)-one (27 mg, 35%) as a solid. LCMS: (ES, m/z): 433.2 [M+H]^{+ 1}HNMR (400 MHz, MeOD) δ ppm 9.64 (d, J=4.02 Hz, 1H), 9.20 (s, 1H), 8.66-8.78 (m, 1H), 8.31-8.41 (m, 1H), 6.86 (dd, J=7.53, 3.01 Hz, 1H), 5.03-5.10 (m, 1H), 4.32 (s, 3H), 3.43 (br d, J=2.76 Hz, 1H), 3.01 (s, 6H), 2.41-2.48 (m, 5H), 2.14 (br d, J=5.14 Hz, 2H), 2.07 (br d, J=15.06 Hz, 2H), 1.94-2.00 (m, 2H).

### Example G107: Synthesis of Compounds 1461 and 1462

### Synthesis of Intermediate G207

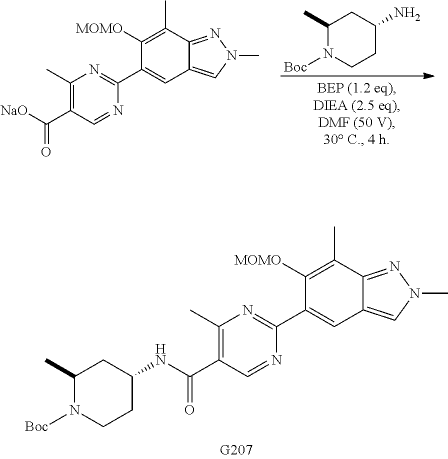

To a mixture of [2-[6-(methoxymethoxy)-2,7-dimethyl-indazol-5-yl]-4-methyl-pyrimidine-5-carbonyl]oxysodium (160 mg, 439 umol, 1 eq) and tert-butyl 4-amino-2-methyl-piperidine-1-carboxylate (113 mg, 527 umol, 1.2 eq) in DMF (8.00 mL) was added 2-bromo-1-ethyl-pyridin-1-ium; tetrafluoroborate (144 mg, 527 umol, 1.2 eq) and DIEA (141 mg, 1.10 mmol, 191 uL, 2.5 eq). The reaction mixture was stirred at 30° C. for 12 h, then quenched with H₂O (10.0 mL) at 0° C., diluted with ethyl acetate (10.0 mL), and the layers separated. The aqueous layer was extracted with ethyl acetate (3×10.0 mL). The organic layers were combined, washed with brine (3×10.0 mL), dried over Na₂SO₄, filtered, and the filtrate concentrated under reduced pressure to give a residue. The residue was purified by column chromatography (SiO₂, Petroleum ether/Ethyl acetate=5/1 to 0/1) to give tert-butyl 4-[[2-[6-(methoxymethoxy)-2,7-dimethyl-indazol-5-yl]-4-methyl-pyrimidine-5-carbonyl]amino]-2-methyl-piperidine-1-carboxylate (140 mg, 59%) as a solid. LCMS: (ES, m/z): 539.3 [M+H]⁺.

### Synthesis of Intermediate G208

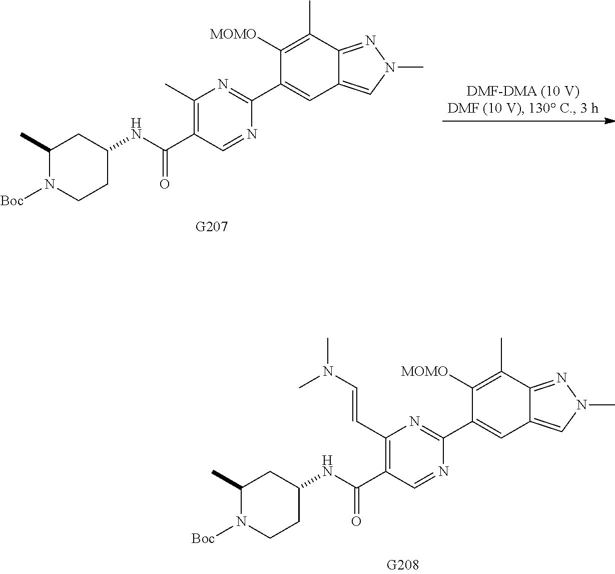

To a solution of tert-butyl 4-[[2-[6-(methoxymethoxy)-2,7-dimethyl-indazol-5-yl]-4-methyl-pyrimidine-5-carbonyl]amino]-2-methyl-piperidine-1-carboxylate (130 mg, 185 umol, 1 eq) in DMF (1.00 mL) was added DMF-DMA (897 mg, 1.00 mL). The reaction mixture was stirred at 130° C. for 3 h, then concentrated under reduced pressure to give tert-butyl 4-[[4-[(Z)-2-(dimethylamino)vinyl]-2-[6-(methoxy-methoxy)-2,7-dimethyl-indazol-5-yl]pyrimidine-5-carbonyl]amino]-2-methyl-piperidine-1-carboxylate (120 mg, 71%) as a solid. LCMS: (ES, m/z): 594.3 [M+H]⁺.

### Synthesis of Intermediate G209

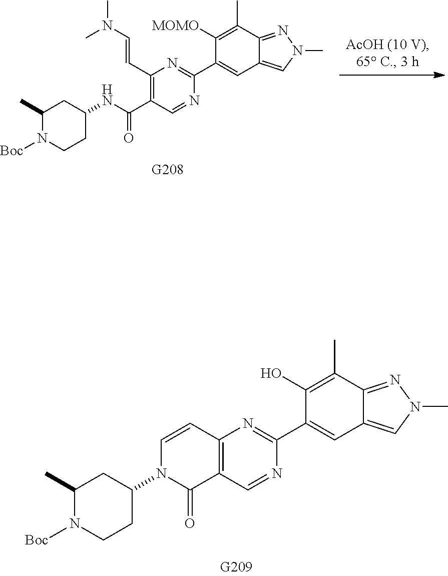

To tert-butyl 4-[[4-[(Z)-2-(dimethylamino) vinyl]-2-[6-(methoxymethoxy)-2,7-dimethyl-indazol-5-yl]pyrimidine-5-carbonyl]amino]-2-methyl-piperidine-1-carboxylate (110 mg, 122 umol, 1 eq) was added acetic acid (762 mg, 726 uL). The reaction mixture was stirred at 65° C. for 3 h, then concentrated under reduced pressure to give a residue. The residue was purified by column chromatography (SiO₂, Petroleum ether/Ethyl acetate=4/1 to 0/1) to give tert-butyl 4-[2-(6-hydroxy-2,7-dimethyl-indazol-5-yl)-5-oxo-pyrido[4,3-d]pyrimidin-6-yl]-2-methyl-piperidine-1-carboxylate (70 mg, 94%) as a solid. LCMS: (ES, m/z): 594.3 [M+H]⁺. ¹H NMR (400 MHz, METHANOL-d₄) δ ppm 9.60 (s, 1H), 9.04 (s, 1H), 8.29 (s, 1H), 7.99 (d, J=7.87 Hz, 1H), 6.77 (d, J=7.63 Hz, 1H), 5.28-5.35 (m, 1H), 4.14-4.21 (m, 4H), 2.44 (s, 3H), 2.01 (s, 3H), 1.50 (s, 9H), 1.46 (d, J=3.70 Hz, 2H), 1.35 (br s, 3H), 1.01 (s, 2H).

### Synthesis of Intermediate G210

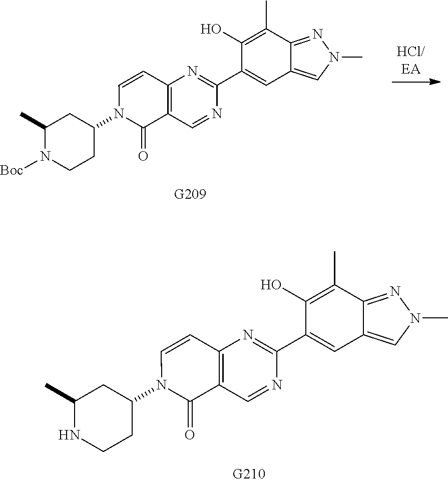

To tert-butyl 4-[2-(6-hydroxy-2,7-dimethyl-indazol-5-yl)-5-oxo-pyrido[4,3-d]pyrimidin-6-yl]-2-methyl-piperidine-1-carboxylate (70.0 mg, 138 umol, 1 eq) was added HCl/ethyl acetate (4 M, 700 uL). The reaction mixture was stirred at 20° C. for 1 h, then concentrated under reduced pressure to give a residue. The residue was triturated with ethyl acetate at 25° C. for 30 min to give a suspension. The suspension was filtered and the filter cake dried to give 2-(6-hydroxy-2,7-dimethyl-indazol-5-yl)-6-(2-methyl-4-piperidyl)pyrido[4,3-d]pyrimidin-5-one (42.0 mg, 74%) as a solid. LCMS: (ES, m/z): 405.1 [M+H]⁺. ¹H NMR (400 MHz, METHANOL-d4) δ ppm 9.67 (s, 1H), 9.26 (s, 1H), 8.84 (s, 1H), 8.00 (d, J=7.75 Hz, 1H), 6.88 (d, J=7.75 Hz, 1H), 5.22-5.37 (m, 1H), 4.35 (s, 3H), 3.97-4.12 (m, 1H), 3.40-3.54 (m, 2H), 2.42-2.52 (m, 4H), 2.28-2.40 (m, 1H), 2.15-2.24 (m, 1H), 2.02-2.13 (m, 1H), 1.58 (d, J=7.15 Hz, 3H).

### Synthesis of Compound 1462 and 1461

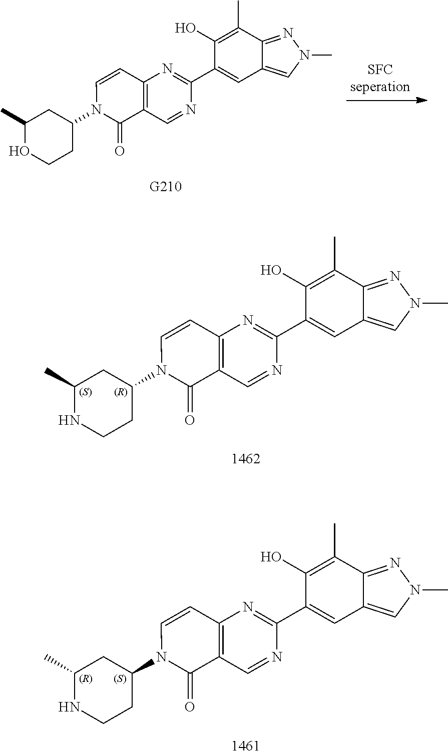

2-(6-hydroxy-2,7-dimethyl-indazol-5-yl)-6-(2-methyl-4-piperidyl)pyrido[4,3-d]pyrimidin-5-one (42.0 mg, 103 umol, 1 eq) was purified by SFC (Condition G3) to give 2-(6-hydroxy-2,7-dimethyl-indazol-5-yl)-6-[(2S,4R)-2-methyl-4-piperidyl]pyrido[4,3-d]pyrimidin-5-one (12.9 mg, 31%) and 2-(6-hydroxy-2,7-dimethyl-indazol-5-yl)-6-[(2R,4S)-2-methyl-4-piperidyl]pyrido[4,3-d]pyrimidin-5-one (12.7 mg, 30%) as solids. 1462: SFC (RT=1.870 min). LCMS: (ES, m/z): 405.3 [M+H]^{+ 1}H NMR (400 MHz, METHANOL-d₄) δ ppm 9.57 (s, 1H), 9.02 (s, 1H), 8.28 (s, 1H), 7.95 (d, J=7.75 Hz, 1H), 6.79 (d, J=7.63 Hz, 1H), 5.18-5.35 (m, 1H), 4.19 (s, 3H), 3.58-3.69 (m, 1H), 3.18-3.26 (m, 1H), 3.07-3.15 (m, 1H), 2.43 (s, 3H), 2.07-2.20 (m, 1H), 1.94-2.05 (m, 2H), 1.83-1.93 (m, 1H), 1.42 (d, J=6.91 Hz, 3H. 1461: SFC (RT=2.859 min). LCMS: (ES, m/z): 405.3 [M+H]^{+ 1}H NMR (400 MHz, METHANOL-d₄) δ ppm 9.59 (s, 1H), 9.04 (s, 1H), 8.29 (s, 1H) 7.97 (d, J=7.39 Hz, 1H) 6.73-6.85 (m, 1H) 5.23-5.31 (m, 1H) 4.19 (s, 3H) 3.55-3.62 (m, 1H) 3.13-3.22 (m, 1H) 3.04-3.11 (m, 1H) 2.44 (s, 3H) 2.06-2.17 (m, 1H) 1.93-2.02 (m, 2H) 1.82-1.91 (m, 1H) 1.40 (d, J=6.91 Hz, 3H).

### Example G108: Synthesis of Compound 1463 and 1464

### Synthesis of Intermediate G211

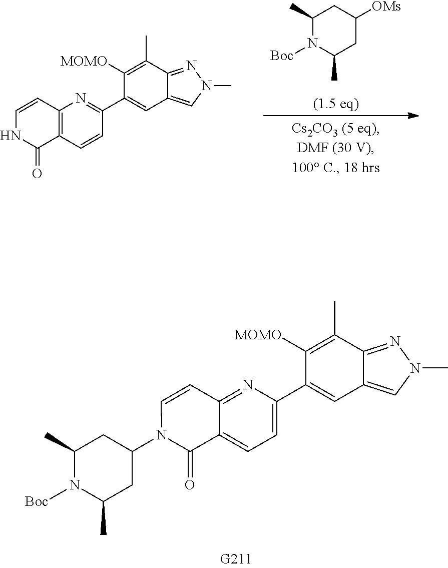

To a mixture of 2-[6-(methoxymethoxy)-2,7-dimethyl-indazol-5-yl]-6H-1,6-naphthyridin-5-one (400 mg, 1.14 mmol, 1.00 eq) and tert-butyl (2R,6S)-2,6-dimethyl-4-methylsulfonyloxy-piperidine-1-carboxylate (525 mg, 1.71 mmol, 1.50 eq) in dimethyl formamide (8.00 mL) was added Cs₂CO₃ (1.11 g, 3.42 mmol, 3.00 eq) at 25° C. under N₂ atmosphere. The reaction mixture was stirred at 100° C. for 18 h, then poured into water (40.0 mL) and extracted with ethyl acetate (3×40.0 mL). The organic layers were combined, washed with brine (100 mL), dried over anhydrous Na₂SO₄, filtered, and the filtrate concentrated under reduced pressure to give a residue. The residue was purified by silica gel column chromatography (ethyl acetate) to give tert-butyl (2R,6S)-4-[2-[6-(methoxymethoxy)-2,7-dimethyl-indazol-5-yl]-5-oxo-1,6-naphthyridin-6-yl]-2,6-dimethyl-piperidine-1-carboxylate (170 mg, 21%) as an oil. LCMS: (ESI, m/z): 562.3 [M+H]⁺. ¹H NMR (400 MHz, DMSO-d₆) δ ppm 8.55 (d, J=8.34 Hz, 1H) 8.43 (s, 1H) 7.89 (s, 1H) 7.84 (d, J=2.50 Hz, 1H) 7.82 (d, J=1.91 Hz, 1H) 6.78 (d, J=7.75 Hz, 1H) 5.41-5.52 (m, 1H) 4.70 (s, 2H) 4.36-4.51 (m, 2H) 4.19 (s, 3H) 3.30 (s, 3H) 3.12 (s, 3H) 2.11 (dt, J=12.25, 6.09 Hz, 2H) 1.73 (br d, J=10.85 Hz, 2H) 1.42-1.46 (m, 9H) 1.30 (br d, J=4.17 Hz, 6H).

### Synthesis of Intermediate G212 and G213

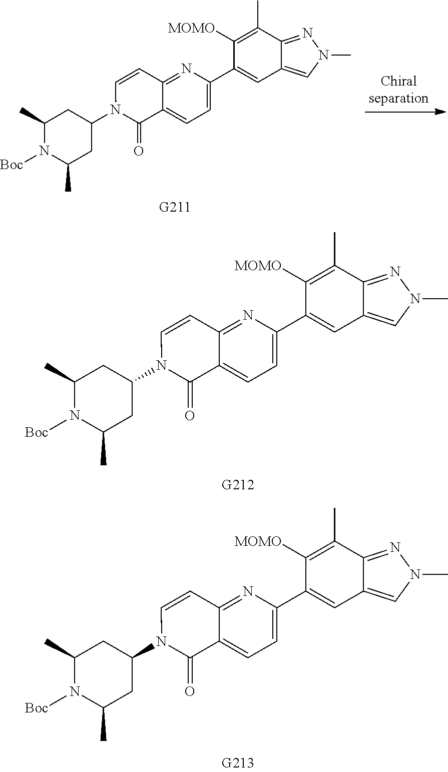

Tert-butyl (2R,6S)-4-[2-[6-(methoxymethoxy)-2,7-dimethyl-indazol-5-yl]-5-oxo-1,6-naphthyridin-6-yl]-2,6-dimethyl-piperidine-1-carboxylate (170 mg) was purified by SFC (Condition G3) to give tert-butyl (2S,6R)-4-[2-(6-hydroxy-2,7-dimethyl-indazol-5-yl)-5-oxo-1,6-naphthyridin-6-yl]-2,6-dimethyl-piperidine-1-carboxylate (140 mg, 82%) and tert-butyl (2S,6R)-4-[2-(6-hydroxy-2,7-dimethyl-indazol-5-yl)-5-oxo-1,6-naphthyridin-6-yl]-2,6-dimethyl-piperidine-1-carboxylate (10.0 mg, 6%) as a solid. G212: LCMS: (ESI, m/z): 562.3 [M+H]⁺. ¹H NMR (400 MHz, METHANOL-d₄) δ ppm 8.70 (d, J=8.44 Hz, 1H) 8.29 (s, 1H) 7.85-7.91 (m, 2H) 7.76 (d, J=7.82 Hz, 1H) 6.92 (d, J=7.82 Hz, 1H) 5.54-5.67 (m, 1H) 4.73 (s, 2H) 4.59 (br t, J=6.60 Hz, 2H) 4.24 (s, 3H) 3.13 (s, 3H) 2.60 (s, 3H) 2.15 (td, J=12.69, 6.17 Hz, 2H) 1.87 (br d, J=12.23 Hz, 2H) 1.51 (s, 9H) 1.41 (br d, J=6.97 Hz, 6H). G213: LCMS: (ESI, m/z): 562.3 [M+H]^{+ 1}H NMR (400 MHz, METHANOL-d₄) δ ppm 8.67 (d, J=8.44 Hz, 1H) 8.29 (s, 1H) 7.83-7.88 (m, 2H) 7.80 (d, J=7.82 Hz, 1H) 6.94 (d, J=7.95 Hz, 1H) 4.93-4.99 (m, 1H) 4.73 (s, 2H) 4.39-4.50 (m, 2H) 4.24 (s, 3H) 3.13 (s, 3H) 2.60 (s, 3H) 2.30-2.43 (m, 2H) 1.97 (td, J=12.56, 7.52 Hz, 2H) 1.52 (s, 9H) 1.41 (d, J=6.72 Hz, 6H).

### Synthesis of Compound 1463

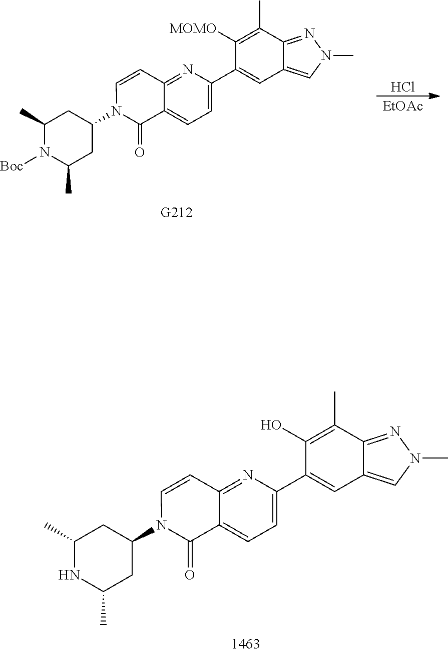

To a solution of tert-butyl (2S,6R)-4-[2-(6-hydroxy-2,7-dimethyl-indazol-5-yl)-5-oxo-1,6-naphthyridin-6-yl]-2,6-dimethyl-piperidine-1-carboxylate (140 mg, 270 umol, 1.00 eq) in ethyl acetate (2.80 mL) was added HCl/ethyl acetate (4.0 M, 2.80 mL) at 25° C. The reaction mixture was stirred at 25° C. for 4 h, then concentrated to give a residue. The residue was triturated with methyl tert-butyl ether (5.00 mL). The mixture was filtered and the filter cake was dried under reduced pressure to give a suspension. The suspension was filtered and the filter cake dried to give 6-[(2S,6R)-2,6-dimethyl-4-piperidyl]-2-(6-hydroxy-2,7-dimethyl-indazol-5-yl)-1,6-naphthyridin-5-one (112 mg, 100%) as a solid. LCMS: (ESI, m/z): 418.2 [M+H]⁺. ¹H NMR (400 MHz, METHANOL-d₄) δ ppm 8.82 (br d, J=9.29 Hz, 1H) 8.69 (br d, J=19.56 Hz, 2H) 8.34-8.41 (m, 1H) 7.93 (br d, J=7.46 Hz, 1H) 6.92 (br d, J=7.09 Hz, 1H) 5.03-5.13 (m, 1H) 4.33 (s, 3H) 3.61-3.73 (m, 2H) 2.55 (br d, J=15.65 Hz, 2H) 2.48 (s, 3H) 2.00-2.15 (m, 2H) 1.41 (br d, J=5.38 Hz, 6H).

### Synthesis of Compound 1464

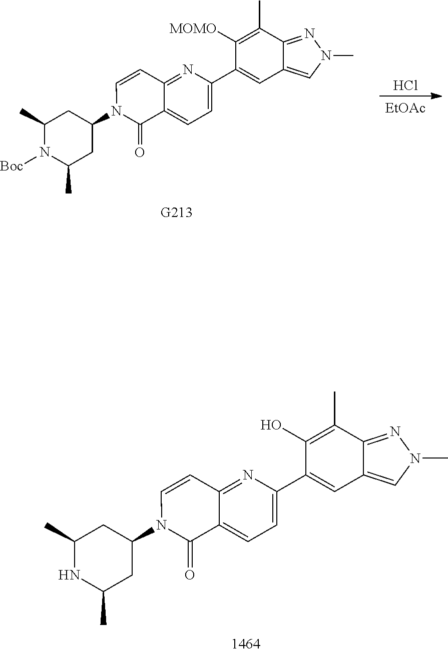

To a solution of tert-butyl (2S,6R)-4-[2-(6-hydroxy-2,7-dimethyl-indazol-5-yl)-5-oxo-1,6-naphthyridin-6-yl]-2,6-dimethyl-piperidine-1-carboxylate (10.0 mg, 19.3 umol, 1.00 eq) in ethyl acetate (0.40 mL) was added HCl/ethyl acetate (4.0 M, 0.40 mL) at 25° C. The reaction mixture was stirred at 25° C. for 4 h, then concentrated to give a residue. The residue was triturated with methyl tert-butyl ether (2.00 mL) to give a suspension. The suspension was filtered and the filter cake was dried under reduced pressure to give 6-[(2S,6R)-2,6-dimethyl-4-piperidyl]-2-(6-hydroxy-2,7-dimethyl-indazol-5-yl)-1,6-naphthyridin-5-one (6.64 mg, 82%) as a solid. LCMS: (ESI, m/z): 418.2 [M+H]⁺. ¹H NMR (400 MHz, METHANOL-d₄) δ ppm 8.79 (d, J=8.80 Hz, 1H) 8.51-8.62 (m, 2H) 8.35 (d, J=9.05 Hz, 1H) 7.71 (d, J=7.82 Hz, 1H) 6.93 (d, J=7.82 Hz, 1H) 5.10-5.19 (m, 1H) 4.28 (s, 3H) 3.50-3.58 (m, 2H) 2.47 (s, 3H) 2.25 (br d, J=14.55 Hz, 2H) 1.97-2.10 (m, 2H) 1.45 (d, J=6.48 Hz, 6H).

### Example G109: Synthesis of Compounds 652 and 653

### Synthesis of Intermediate G214

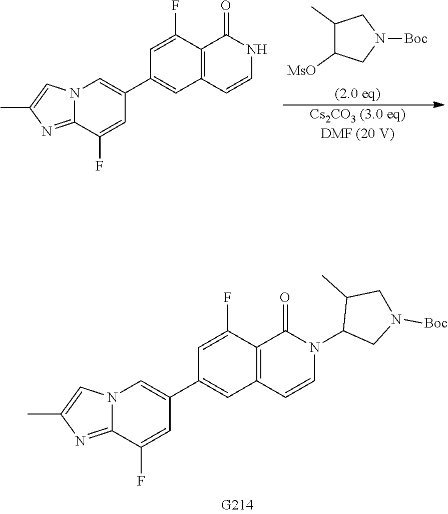

To a solution of 8-fluoro-6-(8-fluoro-2-methyl-imidazo[1,2-a]pyridin-6-yl)-2H-isoquinolin-1-one (500 mg, 1.61 mmol, 1 eq) in DMF (10.0 mL) was added Cs₂CO₃ (1.57 g, 4.82 mmol, 3 eq) and tert-butyl 3-methyl-4-methylsulfonyloxy-pyrrolidine-1-carboxylate (897 mg, 3.21 mmol, 2 eq). The reaction mixture was stirred at 80° C. for 4 h, then quenched with water (10.0 mL), diluted with ethyl acetate (10.0 mL), and the layers separated. The aqueous layer was extracted with ethyl acetate (3×40.0 mL). The organic layers were combined, washed with brine (3×10.0 mL), dried over Na₂SO₄, filtered, and the filtrate concentrated under reduced pressure to give a residue. The residue was purified by prep-HPLC (Condition G8, Gradient 3) to give tert-butyl 3-[8-fluoro-6-(8-fluoro-2-methyl-imidazo[1,2-a]pyridin-6-yl)-1-oxo-2-isoquinolyl]-4-methyl-pyrrolidine-1-carboxylate (100 mg, 9%) as a solid. LCMS: (ES, m/z): 495.3 [M+H]⁺.

### Synthesis of Intermediate G215 and G216

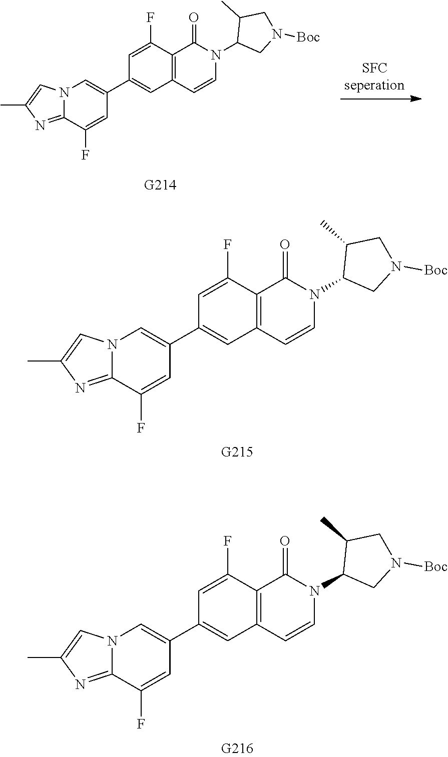 [SMILES: Cc1cn2cc(-c3cc(F)c4c(=O)n(C5CN(C(=O)OC(C)(C)C)CC5C)ccc4c3)cc(F)c2n1 ; Cc1cn2cc(-c3cc(F)c4c(=O)n([C@H]5CN(C(=O)OC(C)(C)C)C[C@H]5C)ccc4c3)cc(F)c2n1 ; Cc1cn2cc(-c3cc(F)c4c(=O)n([C@@H]5CN(C(=O)OC(C)(C)C)C[C@@H]5C)ccc4c3)cc(F)c2n1]

8-fluoro-6-(8-fluoro-2-methyl-imidazo[1,2-a]pyridin-6-yl)-2H-isoquinolin-1-one (500 mg, 1.61 mmol, 1 eq) was purified by SFC (Condition G4) to give tert-butyl 3-[8-fluoro-6-(8-fluoro-2-methyl-imidazo[1,2-a]pyridin-6-yl)-1-oxo-2-isoquinolyl]-4-methyl-pyrrolidine-1-carboxylate (9.20 mg, 15%) and tert-butyl 3-[8-fluoro-6-(8-fluoro-2-methyl-imidazo[1,2-a]pyridin-6-yl)-1-oxo-2-isoquinolyl]-4-methyl-pyrrolidine-1-carboxylate (11.0 mg, 18%) as solids. G215: LCMS: (ES, m/z): 495.1 [M+H]⁺. ¹H NMR (400 MHz, CHLOROFORM-d) δ ppm 8.19 (br s, 1H), 7.39-7.57 (m, 2H), 7.13-7.19 (m, 1H), 7.01-7.11 (m, 1H), 6.38-6.66 (m, 1H), 5.64-5.83 (m, 1H), 3.39-4.11 (m, 4H), 2.94-3.16 (m, 1H), 2.69-2.89 (m, 1H), 2.46-2.62 (m, 3H), 1.53 (br s, 9H), 0.86-0.96 (m, 3H). ¹⁹F NMR (376 MHz, CHLOROFORM-d) δ ppm: −108.259, −129.353. G216: LCMS: (ES, m/z): 495.1 [M+H]⁺. ¹H NMR (400 MHz, CHLOROFORM-d) δ ppm 8.19 (s, 1H), 7.50 (br d, J=2.15 Hz, 1H), 7.43 (br s, 1H), 7.15-7.21 (m, 1H), 7.07 (br d, J=3.22 Hz, 1H), 6.54 (br d, J=6.79 Hz, 1H), 5.70-5.84 (m, 1H), 3.48-4.13 (m, 4H), 3.00-3.16 (m, 1H), 2.69-2.86 (m, 1H), 2.53 (s, 3H), 1.53 (br s, 9H), 0.77-0.87 (m, 3H). ¹⁹F NMR (376 MHz, CHLOROFORM-d) δ ppm: −108.190, −129.072.

### Synthesis of Compound 652

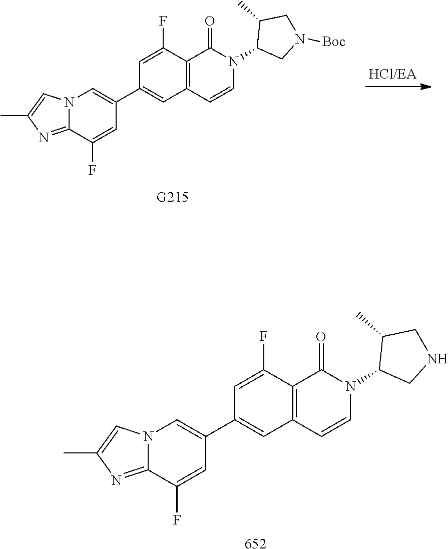 [SMILES: Cc1cn2cc(-c3cc(F)c4c(=O)n([C@H]5CN(C(=O)OC(C)(C)C)C[C@H]5C)ccc4c3)cc(F)c2n1 ; "G215" ; Cc1cn2cc(-c3cc(F)c4c(=O)n([C@H]5CNC[C@H]5C)ccc4c3)cc(F)c2n1 ; "652"]

To a solution of tert-butyl (3R,4R)-3-[8-fluoro-6-(8-fluoro-2-methyl-imidazo[1,2-a]pyridin-6-yl)-1-oxo-2-isoquinolyl]-4-methyl-pyrrolidine-1-carboxylate (9.00 mg, 18.2 umol, 1 eq) in ethyl acetate (200 uL) was added HCl/ethyl acetate (4 M, 200 uL). The reaction mixture was stirred at 25° C. for 2 h, then concentrated under reduced pressure to give a residue. The residue was triturated with dichloromethane at 25° C. for 30 min to give a suspension. The suspension was filtered and the filter cake dried to give 8-fluoro-6-(8-fluoro-2-methyl-imidazo[1,2-a]pyridin-6-yl)-2-[(3R,4R)-4-methylpyrrolidin-3-yl]isoquinolin-1-one (7.85 mg, 98%) as a solid. LCMS: (ES, m/z): 395.2 [M+H]⁺. ¹H NMR (400 MHz, METHANOL-d₄) δ ppm 9.10 (s, 1H) 8.26 (br d, J=11.09 Hz, 1H), 8.12 (br s, 1H), 7.89 (s, 1H), 7.65 (br d, J=12.64 Hz, 1H), 7.30-7.48 (m, 1H), 6.80 (br d, J=7.27 Hz, 1H), 4.07 (br d, J=13.47 Hz, 1H), 3.75-3.90 (m, 1H), 3.57-3.68 (m, 1H), 3.51 (br t, J=10.67 Hz, 1H), 2.86-3.05 (m, 1H), 2.62 (s, 3H), 1.19-1.35 (m, 1H), 0.98 (br d, J=6.56 Hz, 3H). ¹⁹F NMR (376 MHz, METHANOL-d₄) δ ppm: −109.984, −133.609.

### Synthesis of Compound 653

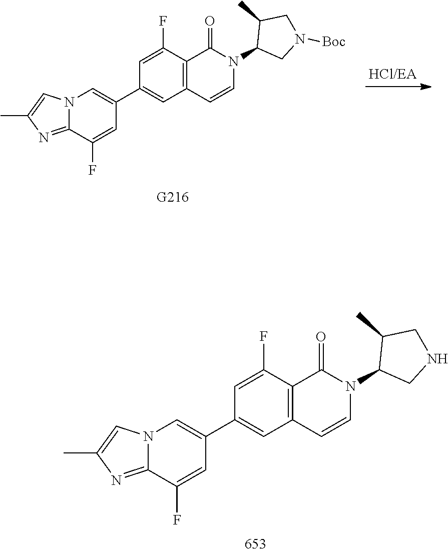 [SMILES: Cc1cn2cc(-c3cc(F)c4c(=O)n([C@@H]5CN(C(=O)OC(C)(C)C)C[C@@H]5C)ccc4c3)cc(F)c2n1 ; "G216" ; Cc1cn2cc(-c3cc(F)c4c(=O)n([C@@H]5CNC[C@@H]5C)ccc4c3)cc(F)c2n1 ; "653"]

To a solution of tert-butyl (3S,4S)-3-[8-fluoro-6-(8-fluoro-2-methyl-imidazo[1,2-a]pyridin-6-yl)-1-oxo-2-isoquinolyl]-4-methyl-pyrrolidine-1-carboxylate (11.0 mg, 22.24 umol, 1 eq) in ethyl acetate (200 uL) was added HCl/ethyl acetate (4 M, 200 uL). The reaction mixture was stirred at 25° C. for 2 h, then concentrated under reduced pressure to give a residue. The residue was triturated with dichloromethane at 25° C. for 30 min to give a suspension. The suspension was filtered and the filter cake dried to give 8-fluoro-6-(8-fluoro-2-methyl-imidazo[1,2-a]pyridin-6-yl)-2-[(3S,4S)-4-methylpyrrolidin-3-yl]isoquinolin-1-one (7.87 mg, 89%) as a solid. LCMS: (ES, m/z): 395.2 [M+H]^{+ 1}H NMR (400 MHz, METHANOL-d₄) δ ppm 9.11 (br s, 1H), 8.25 (br d, J=11.09 Hz, 1H), 8.13 (br s, 1H), 7.89 (br s, 1H), 7.65 (br d, J=12.40 Hz, 1H), 7.40 (br d, J=6.32 Hz, 1H), 6.80 (br d, J=4.77 Hz, 1H), 4.07 (br d, J=12.52 Hz, 1H), 3.76-3.89 (m, 1H), 3.58-3.67 (m, 1H), 3.51 (br t, J=10.49 Hz, 1H), 2.84-3.10 (m, 1H), 2.61 (s, 3H), 1.11-1.40 (m, 1H), 0.98 (br d, J=6.32 Hz, 3H). ¹⁹F NMR (376 MHz, METHANOL-d₄) δ ppm: −109.998, −133.581.

### Example G110: Synthesis of Compound 1473

### Synthesis Intermediate G217

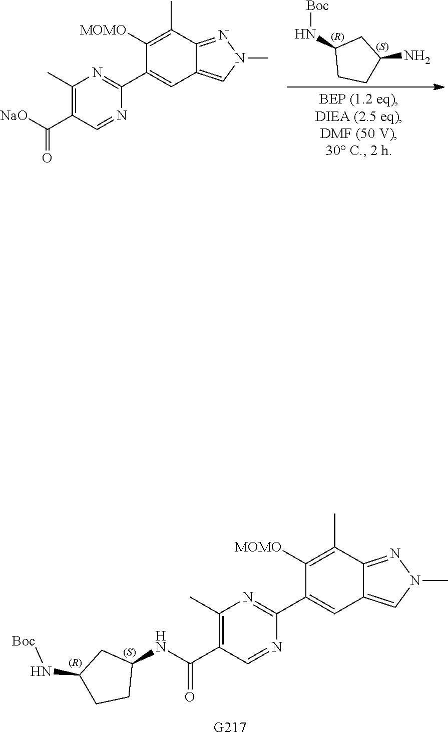

To a mixture of 2-[6-(methoxymethoxy)-2,7-dimethyl-indazol-5-yl]-4-methyl-pyrimidine-5-carboxylic acid (250 mg, 684.30 umol, 1 eq) and tert-butyl N-[(1R,3S)-3-aminocyclopentyl]carbamate (164.46 mg, 821.16 umol, 1.2 eq) in N,N-dimethylformamide (12.5 mL) was added 2-bromo-1-ethyl-pyridin-1-ium tetrafluoroborate (224.88 mg, 821.16 umol, 1.2 eq) and N,N-diisopropylethylamine (221.10 mg, 1.71 mmol, 297.98 uL, 2.5 eq). The reaction mixture was stirred at 30° C. for 2 h, then diluted with water (15 mL) and extracted with ethyl acetate (20×3 mL). The organic layers were combined, washed with brine (50 mL), dried over Na₂SO₄, filtered, and the filtrate concentrated under reduced pressure to give a residue. The residue was purified by column chromatography on silica gel, eluting with 70% ethyl acetate in petroleum ether to give tert-butyl N-[(1R,3S)-3-[[2-[6-(methoxymethoxy)-2,7-dimethyl-indazol-5-yl]-4-methyl-pyrimidine-5-carbonyl]amino]cyclopentyl]carbamate (270 mg, 72%) as a solid. LCMS: (ES, m/z): 525.2 [M+H]^{+ 1}HNMR (400 MHz, CDCl₃) δ ppm 8.85 (s, 1H), 7.96 (d, J=14.78 Hz, 2H), 4.91-4.98 (m, 1H), 4.88 (s, 2H), 4.46-4.55 (m, 1H), 4.24 (s, 3H), 3.81 (br d, J=6.91 Hz, 1H), 3.37 (s, 3H), 2.85 (s, 1H), 2.79 (s, 3H), 2.66 (s, 3H), 2.48 (ddd, J=14.31, 9.42, 7.63 Hz, 1H), 1.83-2.08 (m, 5H) 1.73 (br d, J=14.31 Hz, 1H) 1.42 (s, 8H).

### Synthesis of Intermediate G218

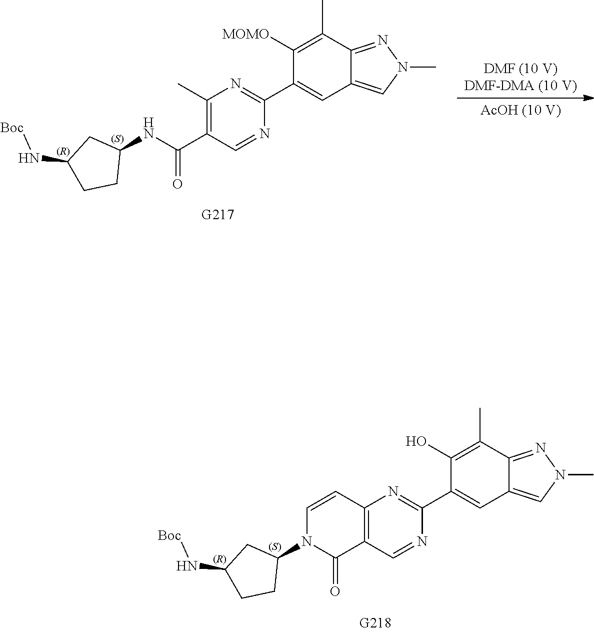

To a solution of tert-butyl N-[(1R,3S)-3-[[2-[6-(methoxymethoxy)-2,7-dimethyl-indazol-5-yl]-4-methyl-pyrimidine-5-carbonyl]amino]cyclopentyl]carbamate (87-1, 100 mg, 190.62 umol, 1 eq) in N,N-dimethylformamide (0.1 mL) was added DMF-DMA (0.1 mL). The reaction mixture was stirred at 120° C. for 2 h, then concentrated under reduced pressure to give a residue. To the residue was added acetic acid (1.00 mL) and the reaction mixture was stirred at 65° C. for 4 h, then filtered. The filter cake was washed with methyl tert-butyl ether (3×1 mL) and dried under vacuum to give a residue. The residue was purified by prep-HPLC (Condition G1, Gradient 7) to give tert-butyl N-[(1R,3S)-3-[2-[6-(methoxymethoxy)-2,7-dimethyl-indazol-5-yl]-5-oxo-pyrido[4,3-d]pyrimidin-6-yl]cyclopentyl]carbamate (50 mg, 49%) as a solid. LCMS: (ES, m/z): 491.2 [M+H]⁺. ¹HNMR (400 MHz, CDCl₃) δ ppm 13.15 (s, 1H), 9.54 (s, 1H), 8.97 (s, 1H), 8.47 (s, 1H), 8.13 (d, J=7.78 Hz, 1H), 7.11 (br d, J=4.14 Hz, 1H), 6.86 (d, J=7.65 Hz, 1H), 5.17 (br t, J=8.72 Hz, 1H), 4.15 (s, 3H), 3.93 (br d, J=7.15 Hz, 1H), 2.38 (s, 3H), 1.60-2.11 (m, 6H), 1.41 (s, 9H).

### Synthesis of Compound 1473

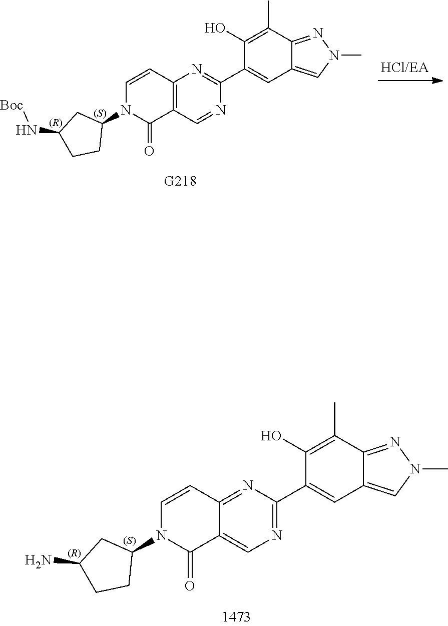

To a solution of tert-butyl N-[(1R,3S)-3-[5-hydroxy-2-(6-hydroxy-2,7-dimethyl-indazol-5-yl)-5H-pyrido[4,3-d]pyrimidin-6-yl]cyclopentyl]carbamate (50 mg, 101.51 umol, 1 eq) in ethyl acetate (0.4 mL) was added HCl/ethyl acetate (4 M, 0.4 mL). The reaction mixture was stirred at 25° C. for 1 h, then concentrated under reduced pressure to give a residue. The residue was triturated with dichloromethane (2 mL) at 20° C. for 5 min to give a suspension. The suspension was filtered and the filter cake was collected with water (2 mL). The aqueous solution was lyophilized to give 6-[(1S,3R)-3-aminocyclopentyl]-2-(6-hydroxy-2,7-dimethyl-indazol-5-yl)pyrido[4,3-d]pyrimidin-5-one (26 mg, 60%) as a solid. LCMS: (ES, m/z): 3911.1 [M+H]⁺. ¹H NMR (400 MHz, D₂O) δ ppm 8.62 (s, 1H), 7.88 (s, 2H), 7.72 (d, J=7.75 Hz, 1H), 6.31 (d, J=7.63 Hz, 1H), 4.92 (br s, 1H), 3.89 (s, 3H), 3.85 (br d, J=7.25 Hz, 1H), 2.63-2.73 (m, 1H), 2.22-2.32 (m, 2H), 1.88-2.18 (m, 4H), 1.81 (s, 3H).

### Example G111: Synthesis of Compound 1476

### Synthesis of Intermediate G219

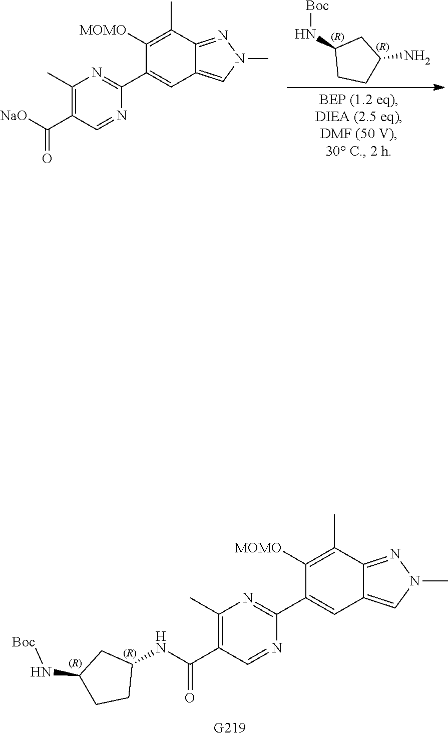

To a solution of [2-[6-(methoxymethoxy)-2,7-dimethyl-indazol-5-yl]-4-methyl-pyrimidine-5-carbonyl]oxysodium (200 mg, 548 umol, 1 eq) in DMF (10.0 mL) was added 2-bromo-1-ethyl-pyridin-1-ium; tetrafluoroborate (180 mg, 658 umol, 1.2 eq), tert-butyl ((1R,3R)-3-aminocyclopentyl)carbamate (136 mg, 658 umol, 1.2 eq) and N-ethyl-N-propan-2-ylpropan-2-amine (177 mg, 1.37 mmol, 239 uL, 2.5 eq). The reaction mixture was stirred at 30° C. for 2 h, then diluted with water (10.0 mL) and extracted with ethyl acetate (3×10.0 mL). The organic layers were combined, washed with brine (3×10.0 mL), dried over Na₂SO₄, filtered, and the filtrate concentrated under reduced pressure to give a residue. The residue was purified by column chromatography (SiO2, Petroleum ether/Ethyl acetate=4/1 to 0/1) to give tert-butyl N-[(1R,3R)-3-[[2-[6-(methoxymethoxy)-2,7-dimethyl-indazol-5-yl]-4-methyl-pyrimidine-5-carbonyl]amino]cyclopentyl]carbamate (190 mg, 66%) as a solid. LCMS: (ES, m/z): 525.3 [M+H]⁺.

### Synthesis of Intermediate G220

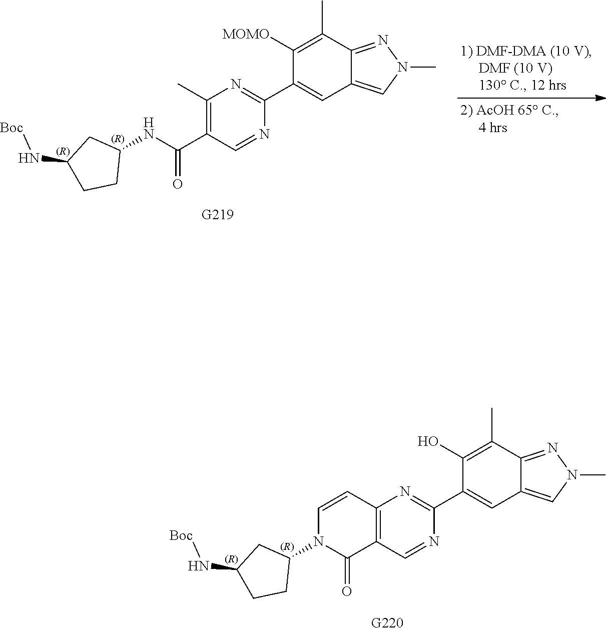

To a solution of tert-butyl N-[(1R,3R)-3-[[2-[6-(methoxymethoxy)-2,7-dimethyl-indazol-5-yl]-4-methyl-pyrimidine-5-carbonyl]amino]cyclopentyl]carbamate (150 mg, 285 umol, 1 eq) in DMF (1.50 mL) was added DMF-DMA (1.34 g, 1.50 mL). The reaction mixture was stirred at 130° C. for 12 h, then concentrated under reduced pressure to give a residue. To the residue was added acetic acid (1.30 mL). The reaction mixture was stirred at 65° C. for 4 h, then concentrated under reduced pressure to give a residue. The residue was purified by column chromatography (SiO2, Petroleum ether/Ethyl acetate=4/1 to 1/1) to give tert-butyl 4-[2-(6-hydroxy-2,7-dimethyl-indazol-5-yl)-5-oxo-pyrido[4,3-d]pyrimidin-6-yl]-2-methyl-piperidine-1-carboxylate (82.0 mg, 58%) as a solid. LCMS: (ES, m/z): 491.3 [M+H]⁺.

### Synthesis of Compound 1476

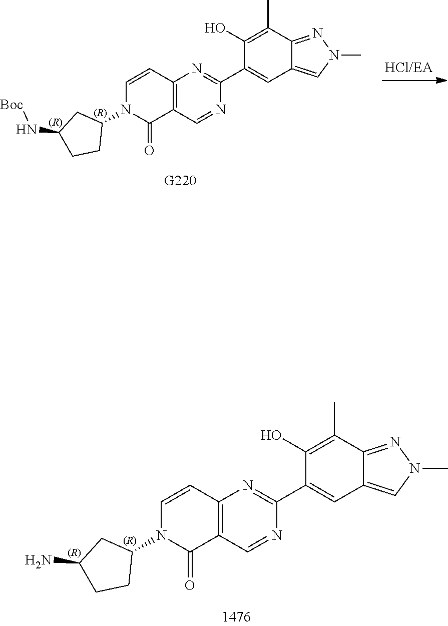

To a solution of tert-butyl N-[(1R,3R)-3-[2-[6-(methoxymethoxy)-2,7-dimethyl-indazol-5-yl]-5-oxo-pyrido[4,3-d]pyrimidin-6-yl]cyclopentyl]carbamate (55.0 mg, 102 umol, 1 eq) in ethyl acetate (1.00 mL) was added HCl/Ethyl acetate (4 M, 1.00 mL). The reaction mixture was stirred at 20° C. for 3 h, then concentrated under reduced pressure to give a residue. The residue was triturated with ethyl acetate (500 uL) at 25° C. for 30 min to give a suspension. The suspension was filtered and the filter cake dried to give 6-[(1R,3R)-3-aminocyclopentyl]-2-(6-hydroxy-2,7-dimethyl-indazol-5-yl)pyrido[4,3-d]pyrimidin-5-one (31.1 mg, 71%) as a solid. LCMS: (ES, m/z): 391.3 [M+H]⁺. ¹H NMR (400 MHz, METHANOL-d₄) δ ppm 9.63 (s, 1H), 9.22 (s, 1H), 8.77 (s, 1H), 8.03 (d, J=7.78 Hz, 1H), 6.83 (d, J=7.65 Hz, 1H), 5.27-5.40 (m, 1H), 4.33 (s, 3H), 4.07 (quin, J=7.43 Hz, 1H), 2.45-2.54 (m, 4H), 2.39-2.45 (m, 1H), 2.32-2.39 (m, 1H), 2.25-2.32 (m, 1H), 2.09-2.23 (m, 1H), 1.81 (ddt, J=13.13, 10.62, 7.89, 7.89 Hz, 1H).

### Example G112: Synthesis of Compound 1478

### Synthesis of Intermediate G221

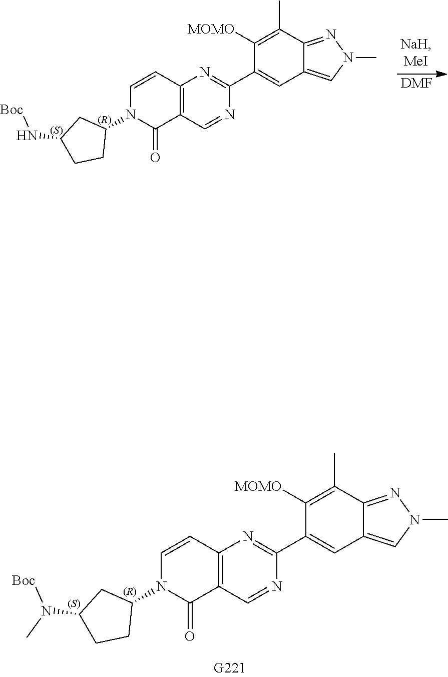

To a solution of tert-butyl N-[(1S,3R)-3-[2-[6-(methoxymethoxy)-2,7-dimethyl-indazol-5-yl]-5-oxo-pyrido[4,3-d]pyrimidin-6-yl]cyclopentyl]carbamate (80 mg, 149.64 umol, 1 eq) in DMF (1 mL) was added NaH (17.96 mg, 448.93 umol, 3 eq) and MeI (106.20 mg, 748.22 umol, 46.58 uL, 5 eq). The reaction mixture was stirred at 25° C. for 4 h, then quenched with water (10 mL) and extracted with ethyl acetate (3×10 mL). The organic layers were combined, dried over MgSO₄, filtered, and the filtrate concentrated under reduced pressure to give a residue, which was purified by prep-HPLC to give tert-butyl N-[(1S,3R)-3-[2-[6-(methoxymethoxy)-2,7-dimethyl-indazol-5-yl]-5-oxo-pyrido[4,3-d]pyrimidin-6-yl]cyclopentyl]-N-methyl-carbamate (12 mg, 13%) as a solid. LCMS: (ES, m/z):549.4 [M+H]⁺. ¹H NMR (CHLOROFORM-d, 400 MHz) δ 9.75 (s, 1H), 8.11 (s, 1H), 7.97 (s, 1H), 7.63 (d, 1H, J=7.9 Hz), 6.83 (d, 1H, J=7.5 Hz), 5.2-5.3 (m, 1H), 4.89 (s, 2H), 4.5-4.6 (m, 1H), 4.25 (s, 3H), 3.31 (s, 3H), 2.88 (s, 3H), 2.67 (s, 3H), 2.2-2.4 (m, 2H), 1.9-2.1 (m, 4H), 1.50 (s, 10H).

### Synthesis of Compound 1478

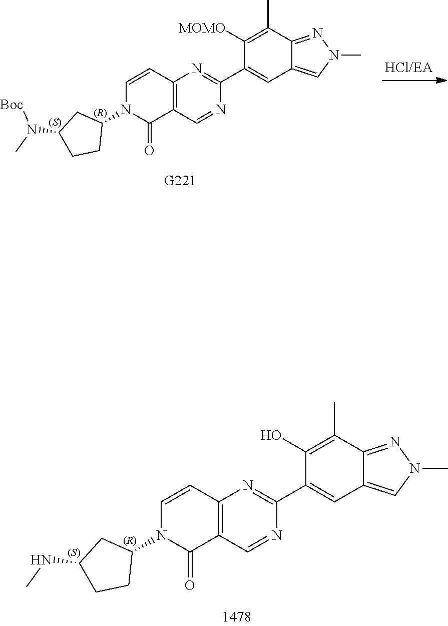

To a solution of tert-butyl N-[(1S,3R)-3-[2-[6-(methoxymethoxy)-2,7-dimethyl-indazol-5-yl]-5-oxo-pyrido[4,3-d]pyrimidin-6-yl]cyclopentyl]-N-methyl-carbamate (12 mg, 21.87 umol, 1 eq) in ethyl acetate (1 mL) was added HCl/EtOAc (4 M, 5.47 uL, 1 eq) under N₂ atmosphere. The reaction mixture was stirred at 25° C. for 6 h, then concentrated under reduced pressure to give a residue. The residue was purified by re-crystallization from methanol (10.0 mL) at 25° C. to give 2-(6-hydroxy-2,7-dimethyl-indazol-5-yl)-6-[(1R,3S)-3-(methylamino)cyclopentyl]pyrido[4,3-d]pyrimidin-5-one (9.06 mg, 88%) as a solid. LCMS: (ES, m/z):405.2 [M+H]⁺. ¹H NMR (METHANOL-d₄, 400 MHz) δ=9.63 (1H, s, M04), 9.10 (1H, s, M03), 8.42 (1H, s, M01), 7.98 (1H, d, J=7.7 Hz, M02), 6.84 (1H, d, J=7.8 Hz, M05), 4.99-5.06 (1H, m, M06), 4.23 (3H, s, M07), 3.71 (1H, quin, J=5.9 Hz, M08), 2.80 (3H, s, M09), 2.62-2.77 (2H, m, M10), 2.45 (3H, s, M11), 2.26-2.33 (2H, m, M13), 2.18-2.24 (2H, m, M12).

### Example G113: Synthesis of Compound 1481

### Synthesis of Intermediate G222

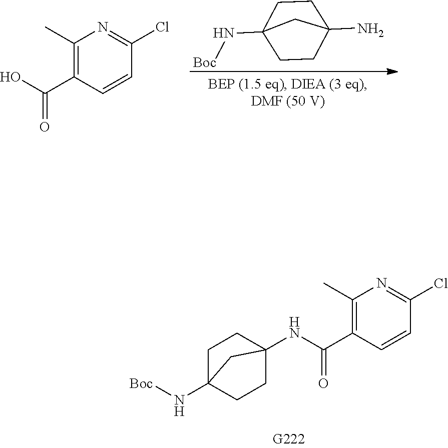 [SMILES: Cc1nc(Cl)ccc1C(=O)O ; CC(C)(C)OC(=O)NC12CCC(N)(CC1)C2 ; Cc1nc(Cl)ccc1C(=O)NC12CCC(NC(=O)OC(C)(C)C)(CC1)C2]

A mixture of 6-chloro-2-methyl-pyridine-3-carboxylic acid (1 g, 5.83 mmol, 1 eq), tert-butyl N-(4-aminonorbornane-1-yl)carbamate (1.32 g, 5.83 mmol, 1 eq), N-ethyl-N-propan-2-ylpropan-2-amine (1.88 g, 14.5 mmol, 2.54 mL, 2.5 eq), and 2-bromo-1-ethyl-pyridin-1-ium; tetrafluoroborate (1.92 g, 6.99 mmol, 1.2 eq) in DMF (55.0 mL) was degassed and purged with N₂ 3 time. The reaction mixture was stirred at 30° C. for 4 h under N₂ atmosphere, then quenched with water (10.0 mL) at 0° C., and extracted with ethyl acetate (3×10.0 mL). The organic layers were combined, washed with brine (3×10 mL), dried over Na₂SO₄, filtered, and the filtrate concentrated under reduced pressure to give a residue. The residue was purified by column chromatography (SiO₂, Petroleum ether/Ethyl acetate=5/1 to 0/1) to give 6-chloro-N-[4-(dimethylamino)cyclohexyl]-2-methyl-pyridine-3-carboxamide (1.70 g, 77%) as a solid. LCMS: (ES, m/z): 380.2 [M+H]⁺. ¹H NMR (400 MHz, CHLOROFORM-d) δ ppm 7.61 (d, J=7.99 Hz, 1H), 7.18 (d, J=7.99 Hz, 1H), 5.89 (s, 1H), 4.75 (br s, 1H), 2.62 (s, 3H), 2.21 (s, 2H), 2.03-2.15 (m, 4H), 1.91-2.01 (m, 2H), 1.74-1.87 (m, 2H), 1.43-1.47 (m, 9H).

### Synthesis of Intermediate G223

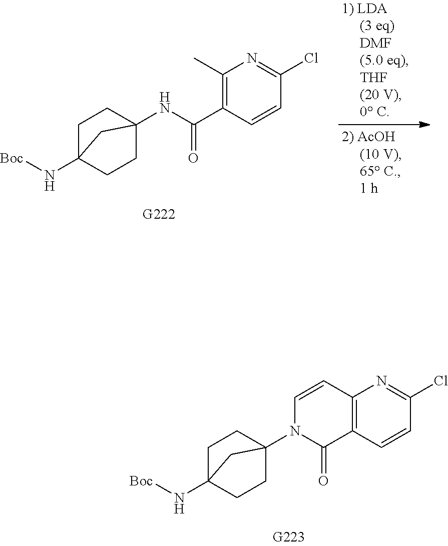 [SMILES: Cc1nc(Cl)ccc1C(=O)NC12CCC(NC(=O)OC(C)(C)C)(CC1)C2 ; CC(C)(C)OC(=O)NC12CCC(n3ccc4nc(Cl)ccc4c3=O)(CC1)C2]

To a solution of tert-butyl N-[4-[(6-chloro-2-methyl-pyridine-3-carbonyl)amino]norbornan-1-yl]carbamate (1.70 g, 4.47 mmol, 1 eq) in THE (30.0 mL) was added LDA (2 M, 6.70 mL, 3 eq) at 0° C. dropwise over 5 min. The reaction mixture was stirred at 0° C. for 1 h, then DMF (3.26 g, 44.7 mmol, 10 eq) was added dropwise at 0° C. The resulting mixture was stirred at 0° C. for 1 h, then quenched with water (10.0 mL) at 0° C., diluted with dichloromethane (10.0 mL), and the layers separated. The aqueous layer was extracted with dichloromethane (3×10.0 mL). The organic layers were combined, washed with brine (3×10.0 mL), dried over Na₂SO₄, filtered, and the filtrate concentrated under reduced pressure to give a residue. To the residue was added acetic acid (15.7 g, 262 mmol, 15.0 mL). The reaction mixture was stirred at 65° C. for 1 h, then concentrated under reduced pressure to give a residue. The residue was purified by column chromatography (SiO₂, Petroleum ether/Ethyl acetate=5/1 to 0/1) to give tert-butyl N-[4-(2-chloro-5-oxo-1,6-naphthyridin-6-yl)norbornan-1-yl]carbamate (630 mg, 36%) as a solid. LCMS: (ES, m/z): 390.2 [M+H]⁺. ¹H NMR (400 MHz, CHLOROFORM-d) δ ppm 8.55 (d, J=8.41 Hz, 1H), 7.50 (d, J=7.91 Hz, 1H), 7.35 (d, J=8.41 Hz, 1H), 6.62 (d, J=7.78 Hz, 1H), 4.78 (br s, 1H), 2.67-2.91 (m, 2H), 2.47 (br s, 2H), 2.10-2.28 (m, 2H), 1.72-1.95 (m, 2H), 1.64 (br s, 2H), 1.47 (s, 9H).

### Synthesis of Intermediate G224

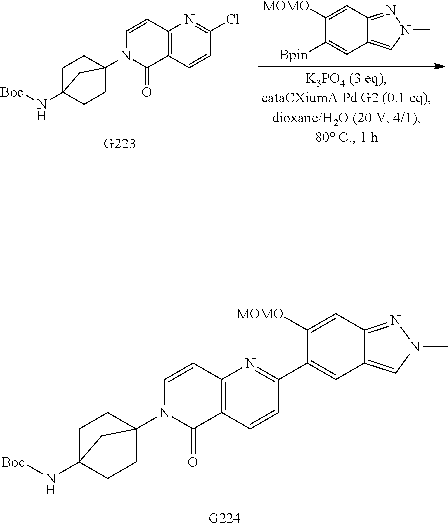

A mixture of tert-butyl N-[4-(2-chloro-5-oxo-1,6-naphthyridin-6-yl)norbornan-1-yl]carbamate (0127-2, 112 mg, 287 umol, 1 eq), 6-(methoxymethoxy)-2-methyl-5-(4,4,5,5-tetramethyl-1,3,2-dioxaborolan-2-yl)indazole (91.4 mg, 287 umol, 1 eq), [2-(2-aminophenyl)phenyl]-chloro-palladium; bis(1-adamantyl)-butyl-phosphane (19.1 mg, 28.7 umol, 0.1 eq), and K₃PO₄ (182 mg, 861 umol, 3 eq) in dioxane (1.50 mL) and water (400 uL) was degassed and purged with N₂ 3 times, then stirred at 80° C. for 1 h under N₂ atmosphere. The reaction mixture was filtered through celite and the filtrate was concentrated under reduced pressure to give a residue. The residue was purified by prep-TLC (Petroleum ether/Ethyl acetate=0:1) to give tert-butyl N-[4-[2-[6-(methoxymethoxy)-2-methyl-indazol-5-yl]-5-oxo-1,6-naphthyridin-6-yl]norbornan-1-yl]carbamate (120 mg, 76%) as a solid. LCMS: (ES, m/z): 546.5 [M+H]⁺.

### Synthesis of Compound 1481

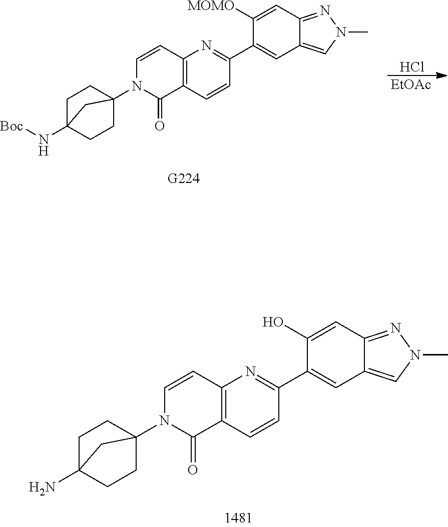

To a solution of tert-butyl N-[4-[2-[6-(methoxymethoxy)-2-methyl-indazol-5-yl]-5-oxo-1,6-naphthyridin-6-yl]norbornan-1-yl]carbamate (120 mg, 220 umol, 1 eq) in ethyl acetate (2.00 mL) was added HCl/ethyl acetate (4 M, 1.00 mL). The reaction mixture was stirred at 25° C. for 3 h, then concentrated under reduced pressure to give a residue. The residue was triturated with ethyl acetate (500 uL) at 25° C. for 30 min to give a suspension. The suspension was filtered and the filter cake dried to give 6-(4-aminonorbornane-1-yl)-2-(6-hydroxy-2-methyl-indazol-5-yl)-1,6-naphthyridin-5-one (56.1 mg, 63%) as a solid. LCMS: (ES, m/z): 402.2 [M+H]⁺. ¹H NMR (400 MHz, METHANOL-d₄) δ ppm 8.97 (d, J=8.63 Hz, 1H), 8.53 (d, J=4.88 Hz, 2H), 8.29 (d, J=8.75 Hz, 1H), 7.93 (d, J=7.88 Hz, 1H), 7.07 (s, 1H), 6.87 (d, J=7.88 Hz, 1H), 4.25 (s, 3H), 2.69-2.82 (m, 2H), 2.57 (s, 2H), 1.97-2.24 (m, 6H).

### Example G114: Synthesis of Compounds 1484 and 1485

### Synthesis of Intermediate G225

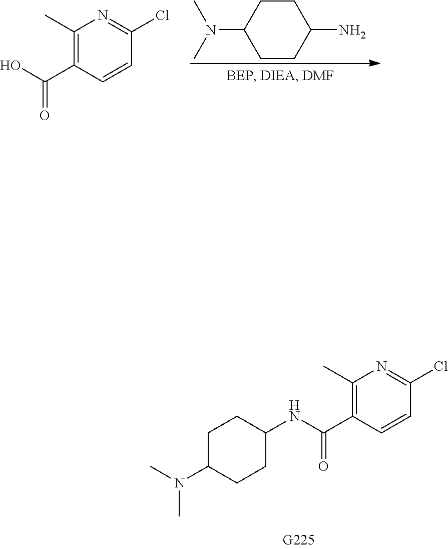

A mixture of 6-chloro-2-methyl-pyridine-3-carboxylic acid (200 mg, 1.17 mmol, 1 eq), N,N-dimethylcyclohexane-1,4-diamine (198 mg, 1.40 mmol, 1.2 eq), 2-bromo-1-ethyl-pyridin-1-ium; tetrafluoroborate (383 mg, 1.40 mmol, 1.2 eq), and N-ethyl-N-propan-2-ylpropan-2-amine (376 mg, 2.91 mmol, 507 uL, 2.5 eq) in DMF (10.0 mL) was degassed and purged with N₂ 3 times, then stirred at 20° C. for 3 h under N₂ atmosphere. The reaction mixture was quenched with water (10.0 mL) at 0° C., then diluted with ethyl acetate (10.0 mL), and the layers separated. The aqueous layer was extracted with ethyl acetate 30.0 mL (3×10.0 mL). The organic layers were combined, washed with brine (3×10.0 mL), dried over Na₂SO₄, filtered, and the filtrate concentrated under reduced pressure to give a residue. The residue was triturated with methyl tert-butyl ether (20.0 mL) for 60 min to give a suspension. The suspension was filtered and the filter cake 6-chloro-N-[4-(dimethylamino)cyclohexyl]-2-methyl-pyridine-3-carboxamide (230 mg, 67%) as a solid. LCMS: (ES, m/z): 296.2 [M+H]⁺.

### Synthesis of Intermediate G226

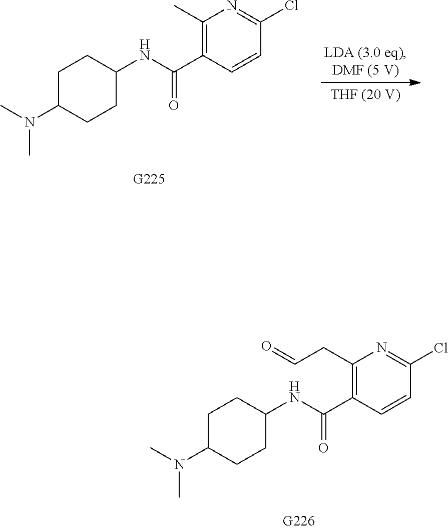 [SMILES: Cc1nc(Cl)ccc1C(=O)NC1CCC(N(C)C)CC1 ; CN(C)C1CCC(NC(=O)c2ccc(Cl)nc2CC=O)CC1]

To a solution of 6-chloro-N-[4-(dimethylamino)cyclohexyl]-2-methyl-pyridine-3-carboxamide (100 mg, 338 umol, 1 eq) in THE (2.00 mL) was added LDA (2 M, 507 uL, 3 eq). The reaction mixture was stirred at 0° C. for 1 h, then DMF (475 mg, 500 uL) was added. The resulting mixture was stirred for an additional 2 h, then quenched with water (20.0 mL) at 0° C., diluted with dichloromethane (10.0 mL), and the layers separated. The aqueous layer was extracted with dichloromethane (3×10.0 mL). The organic layers were combined, washed with brine (3×10.0 mL), dried over Na₂SO₄, filtered, and the filtrate concentrated under reduced pressure to give a residue. The residue 6-chloro-N-[4-(dimethylamino)cyclohexyl]-2-(2-oxoethyl)pyridine-3-carboxamide (60.0 mg, 33%). LCMS: (ES, m/z): 324.1 [M+H]⁺.

### Synthesis of Intermediate G227

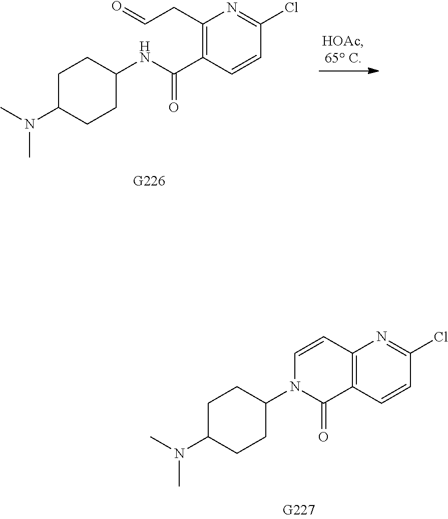 [SMILES: CN(C)C1CCC(NC(=O)c2ccc(Cl)nc2CC=O)CC1 ; CN(C)C1CCC(n2ccc3nc(Cl)ccc3c2=O)CC1]

A mixture of 6-chloro-N-[4-(dimethylamino)cyclohexyl]-2-(2-oxoethyl)pyridine-3-carboxamide (150 mg, 463 umol, 1 eq) in acetic acid (1.50 mL) was degassed and purged with N₂ 3 times, then stirred at 65° C. for 2 h under N₂ atmosphere. The reaction mixture was concentrated under reduced pressure to give a residue. The residue was purified by prep-HPLC (column: Waters Xbridge Prep OBD C18 150*40 mm*10 um; mobile phase: [water(NH₄HCO₃)-ACN]; B %: 15%-45%, 8 min) to give 2-chloro-6-[4-(dimethylamino)cyclohexyl]-1,6-naphthyridin-5-one (40-7, 65.0 mg, 23%) as a solid. LCMS: (ES, m/z): 306.1 [M+H]^{+ 1}H NMR (400 MHz, CHLOROFORM-d) δ ppm 8.62 (d, J=8.25 Hz, 1H), 7.54-7.69 (m, 1H), 7.36 (d, J=8.38 Hz, 1H), 6.73 (br d, J=7.88 Hz, 1H), 4.92-5.20 (m, 1H), 2.83-3.05 (m, 1H), 2.18-2.48 (m, 6H), 2.08-2.18 (m, 2H), 1.92-2.04 (m, 2H), 1.65 (br dd, J=14.13, 3.50 Hz, 2H), 1.16-1.32 (m, 2H).

### Synthesis of Compound 1484 and 1485

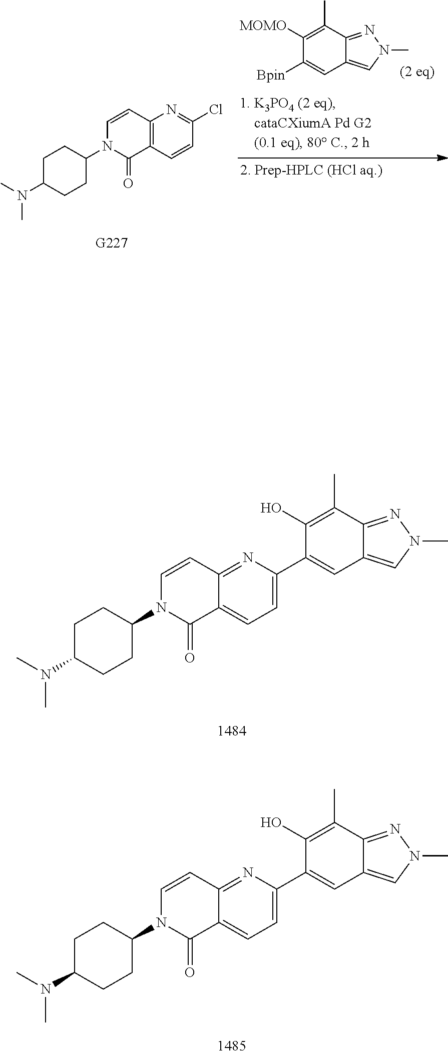

A mixture of 2-chloro-6-[4-(dimethylamino)cyclohexyl]-1,6-naphthyridin-5-one (40-7, 65.0 mg, 212 umol, 1 eq), 6-(methoxymethoxy)-2,7-dimethyl-5-(4,4,5,5-tetra-methyl-1,3,2-dioxaborolan-2-yl)-2H-indazole (46.8 mg, 212 umol, 1 eq), [2-(2-amino-phenyl)phenyl]-chloro-palladium; bis(1-adamantyl)-butyl-phosphane (14.2 mg, 21.2 umol, 0.1 eq), and K₃PO₄ (90.2 mg, 425 umol, 2 eq) in dioxane (800 uL) and water (200 uL) was degassed and purged with N₂ 3 times, then stirred at 80° C. for 2 h under N₂ atmosphere. The reaction mixture was filtered through celite and concentrated under reduced pressure to give a residue. The residue was purified by prep-HPLC (Condition G8, Gradient 6) to give 6-[4-(dimethyl amino)cyclohexyl]-2-(6-hydroxy-2,7-dimethyl-indazol-5-yl)-1,6-naphthyridin-5-one (17.0 mg, 19%) and 6-[4-(dimethylamino)cyclohexyl]-2-(6-hydroxy-2,7-dimethyl-indazol-5-yl)-1,6-naphthyridin-5-one (30.0 mg, 33%) as a solid. 1484: LCMS: (ES, m/z): 432.3 [M+H]^{+ 1}H NMR (400 MHz, METHANOL-d₄) δ ppm 8.84 (d, J=8.76 Hz, 1H), 8.74 (s, 1H), 8.66 (s, 1H), 8.36 (d, J=8.88 Hz, 1H), 7.85 (d, J=7.88 Hz, 1H), 6.93 (d, J=7.63 Hz, 1H), 4.93-4.96 (m, 1H), 4.33 (s, 3H), 3.37-3.44 (m, 1H), 2.93 (s, 6H), 2.48 (s, 3H), 2.31 (br d, J=11.88 Hz, 2H), 2.14 (br d, J=9.01 Hz, 2H), 1.95-2.08 (m, 2H), 1.79-1.93 (m, 2H). 1485: LCMS: (ES, m/z): 432.3 [M+H]^{+ 1}H NMR (400 MHz, METHANOL-d₄) δ ppm 8.81 (d, J=8.78 Hz, 1H), 8.60 (s, 2H), 8.34 (d, J=8.91 Hz, 1 H), 8.06 (d, J=7.78 Hz, 1H), 6.94 (d, J=7.78 Hz, 1H), 5.07 (td, J=10.48, 4.27 Hz, 1H), 4.29 (s, 3H), 3.43 (br s, 1H), 3.02 (s, 6H), 2.36-2.53 (m, 5H), 1.93-2.18 (m, 6H).

### Example G115: Synthesis of Compound 1486

### Synthesis of Intermediate G228

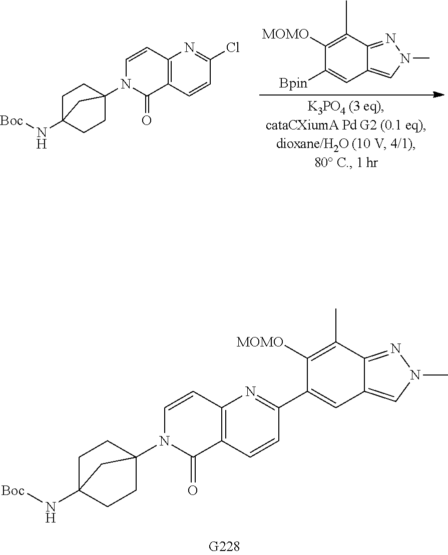

A mixture of tert-butyl N-[4-(2-chloro-5-oxo-1,6-naphthyridin-6-yl)norbornan-1-yl]carbamate (100 mg, 256 umol, 1 eq), 6-(methoxymethoxy)-2,7-dimethyl-5-(4,4,5,5-tetramethyl-1,3,2-dioxaborolan-2-yl)indazole (255 mg, 307 umol, 40% purity, 1.2 eq), [2-(2-aminophenyl)phenyl]-chloro-palladium; bis(1-adamantyl)-butyl-phosphane (17.1 mg, 25.6 umol, 0.1 eq), and K₃PO₄ (163 mg, 769 umol, 3 eq) in dioxane (2.00 mL) and H₂O (500 uL) was degassed and purged with N₂ 3 times, then was stirred at 80° C. for 1 h under N₂ atmosphere. The reaction mixture was quenched with water (10.0 mL) at 0° C., and extracted with ethyl acetate (3×20.0 mL). The organic layers were combined, dried over Na₂SO₄, filtered and the filtrate concentrated under reduced pressure to give a residue. The residue was purified by column chromatography (SiO₂, Petroleum ether/Ethyl acetate=6/1 to 0/1) to give tert-butyl N-[4-[2-[6-(methoxymethoxy)-2,7-dimethyl-indazol-5-yl]-5-oxo-1,6-naphthyridin-6-yl]norbornan-1-yl]carbamate (100 mg, 69%) as solid. LCMS: (ES, m/z): 560.4 [M+H]⁺

### Synthesis of Compound 1486

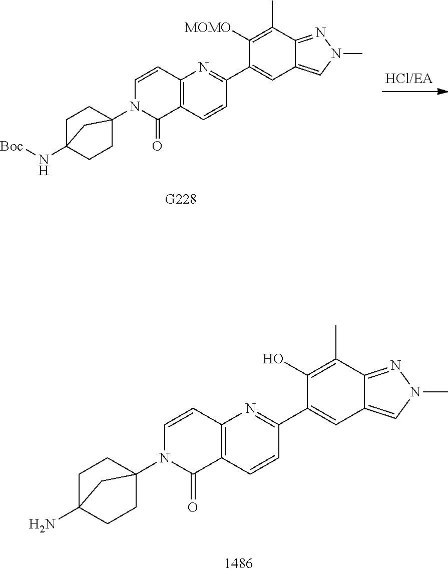

To a solution of tert-butyl N-[4-[2-[6-(methoxymethoxy)-2,7-dimethyl-indazol-5-yl]-5-oxo-1,6-naphthyridin-6-yl]norbornan-1-yl]carbamate (100 mg, 160 umol, 1 eq) in ethyl acetate (1.00 mL) was added HCl/ethyl acetate (4 M, 1.00 mL). The reaction mixture was stirred at 25° C. for 2 h, then concentrated under reduced pressure to give a residue. The residue was triturated with ethyl acetate (500 uL) at 25° C. for 30 min to give a suspension. The suspension was filtered and the filter cake dried to give 6-(4-aminonorbornane-1-yl)-2-(6-hydroxy-2-methyl-indazol-5-yl)-1,6-naphthyridin-5-one (55.0 mg, 74%) as a solid. LCMS: (ES, m/z): 416.4 [M+H]^{+ 1}H NMR (400 MHz, METHANOL-d₄) δ ppm 8.77 (d, J=8.78 Hz, 1H), 8.66 (s, 1H), 8.63 (s, 1H), 8.34 (d, J=8.78 Hz, 1H), 7.79 (d, J=7.91 Hz, 1H), 6.84 (d, J=7.78 Hz, 1H), 4.31 (s, 3H), 2.70-2.82 (m, 2H), 2.56 (s, 2H), 2.48 (s, 3H), 1.95-2.26 (m, 6H).

### Example G116: Synthesis of Compound 1487

### Synthesis Intermediate G229

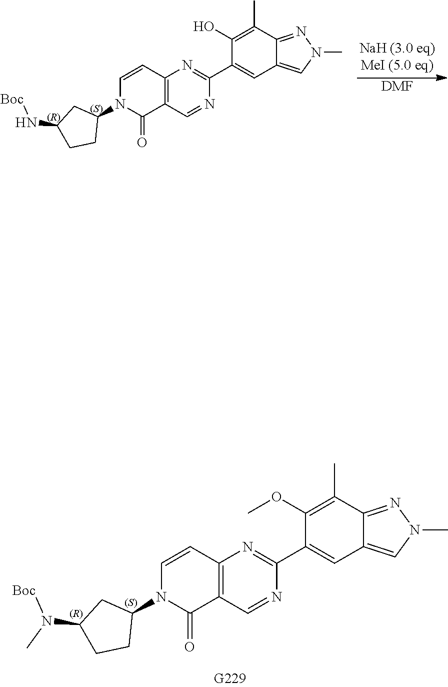

To a solution of tert-butyl N-[(1R,3S)-3-[2-(6-hydroxy-2,7-dimethyl-indazol-5-yl)-5-oxo-pyrido[4,3-d]pyrimidin-6-yl]cyclopentyl]carbamate (90 mg, 203.85 umol, 1 eq) in N,N-dimethylformamide (1 mL) was added sodium hydrogen (24.46 mg, 611.55 umol, 3 eq) at 0° C. The reaction mixture was stirred at 0° C. for 30 min. To the reaction mixture was added methyl iodide (144.67 mg, 1.02 mmol, 63.45 uL, 5 eq) and the resulting mixture was stirred at 20° C. for 2 h. The reaction mixture was quenched with water (5 mL) and extracted with ethyl acetate (20 mL×3). The organic layers were combined, washed with a saturated aqueous sodium chloride solution, dried over anhydrous sodium sulfate, filtered, and the filtrate concentrated under reduced pressure to give a residue. The residue was purified by column chromatography on silica gel, eluting with 70% ethyl acetate in petroleum ether to give tert-butyl N-[(1R,3S)-3-[2-(6-methoxy-2,7-dimethyl-indazol-5-yl)-5-oxo-pyrido[4,3-d]pyrimidin-6-yl]cyclopentyl]carbamate (36 mg, 37%) as a solid. LCMS: (ES, m/z): 519.3 [M+H]⁺. ¹H NMR (DMSO-d₆, 400 MHz): δ (ppm) 9.54 (s, 1H), 8.43 (s, 1H), 8.17 (br d, J=7.7 Hz, 1H), 8.02 (s, 1H), 6.74 (br d, J=7.7 Hz, 1H), 5.09-5.22 (m, 1H), 4.52 (br d, J=2.8 Hz, 1H), 4.18 (s, 3H), 3.68 (s, 3H), 2.80 (s, 3H), 2.47 (br s, 3H), 1.82-2.13 (m, 6H), 1.42 (s, 9H).

### Synthesis of Compound 1487

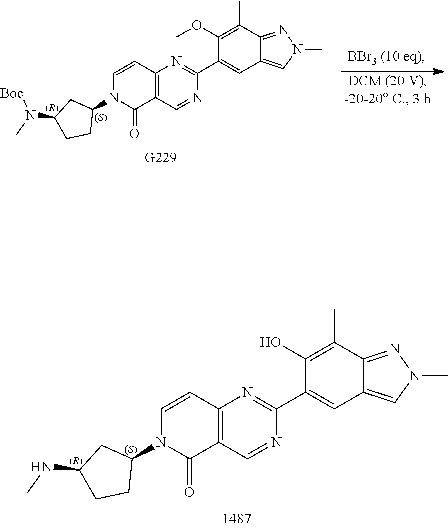

To a solution of tert-butyl N-[(1R,3S)-3-[2-(6-methoxy-2,7-dimethyl-indazol-5-yl)-5-oxo-pyrido[4,3-d]pyrimidin-6-yl]cyclopentyl]-N-methyl-carbamate (36 mg, 69.42 umol, 1 eq) in dichloromethane (0.5 mL) was added boron tribromide (1 M, 240.00 uL, 10.37 eq) at −20° C. The reaction mixture was stirred at 20° C. for 3 h, then dried under nitrogen to give a residue. The residue was purified by prep-HPLC to give 2-(6-hydroxy-2,7-dimethyl-indazol-5-yl)-6-[(1S,3R)-3-(methylamino)cyclopentyl]pyrido[4,3-d]pyrimidin-5-one (2 mg, 7%) as a solid. LCMS: (ES, m/z): 405.2 [M+H]⁺. ¹H NMR (400 MHz, D₂O) δ ppm 8.74 (s, 1H), 7.91-8.07 (m, 2H), 7.75 (d, J=7.65 Hz, 1H), 6.39 (d, J=7.53 Hz, 1H), 4.94 (br d, J=7.03 Hz, 1H), 3.94 (s, 3H), 3.71-3.78 (m, 1H), 2.78 (s, 3H), 2.68-2.74 (m, 1H), 2.30 (br dd, J=7.97, 3.58 Hz, 2H), 1.96-2.18 (m, 3H), 1.89 (s, 3H).

### Example G117: Synthesis of Compound 1489

### Synthesis Intermediate G230

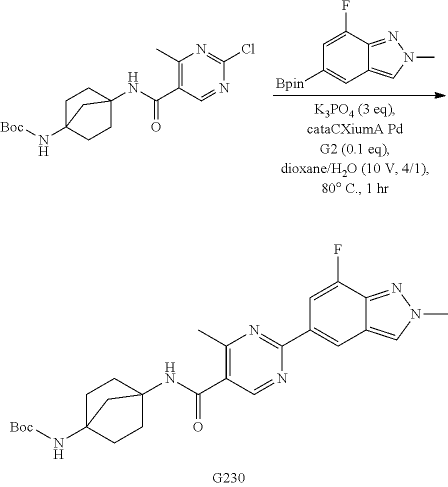

A mixture of tert-butyl N-[4-[(2-chloro-4-methyl-pyrimidine-5-carbonyl)amino]norbornan-1-yl]carbamate (200 mg, 525.12 umol, 1 eq), 7-fluoro-2-methyl-5-(4,4,5,5-tetramethyl-1,3,2-dioxaborolan-2-yl)indazole (217.49 mg, 630.14 umol, 1.2 eq), potassium phosphate (222.93 mg, 1.05 mmol, 2 eq), and [2-(2-aminophenyl)phenyl]-chloro-palladium; bis(1-adamantyl)-butyl-phosphane (35.11 mg, 52.51 umol, 0.1 eq) in dioxane (1.6 mL) and water (0.4 mL) was degassed and purged with nitrogen 3 times, then stirred at 80° C. for 1 h under nitrogen atmosphere. The reaction mixture was diluted with water (20 mL) and extracted with ethyl acetate (30 mL×3). The organic layers were combined, washed with brine (100 mL), dried over anhydrous sodium sulfate, filtered, and the filtrate concentrated under reduced pressure to give a residue. The residue was triturated with dichloromethane (10 mL) at 20° C. for 5 min to give a suspension. The suspension was filtered and the filter cake was dried under vacuum to give tert-butyl N-[4-[[2-(7-fluoro-2-methyl-indazol-5-yl)-4-methyl-pyrimidine-5-carbonyl]amino]norbornan-1-yl]carbamate (210 mg 70%) as a solid. LCMS: (ES, m/z): 495.2 [M+H]⁺. ¹HNMR (400 MHz, DMSO-d6) δ ppm 8.70-8.76 (m, 2H), 8.67 (s, 2H), 7.97 (d, J=13.20 Hz, 1H), 6.91-7.12 (m, 1H), 4.24 (s, 3H), 2.59 (s, 3H), 2.02 (s, 2H), 1.80-1.99 (m, 6H), 1.69 (br d, J=9.29 Hz, 2H), 1.39 (s, 9H).

### Synthesis of Intermediate G231

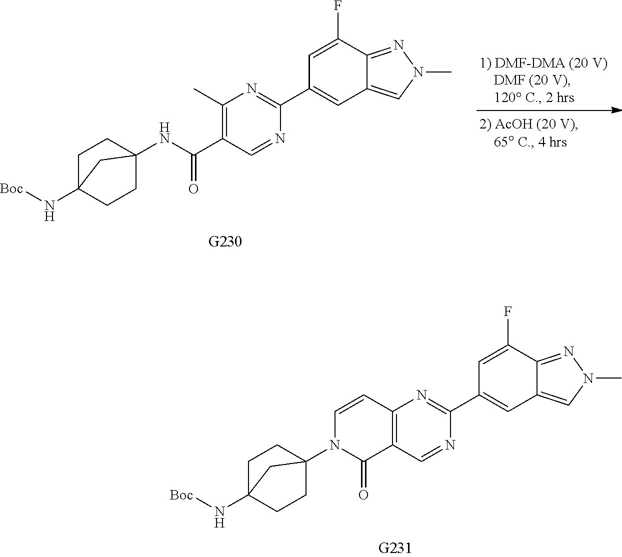 [SMILES: Cc1nc(-c2cc(F)c3nn(C)cc3c2)ncc1C(=O)NC12CCC(NC(=O)OC(C)(C)C)(CC1)C2 ; Cn1cc2cc(-c3ncc4c(=O)n(C56CCC(NC(=O)OC(C)(C)C)(CC5)C6)ccc4n3)cc(F)c2n1]

To a solution of tert-butyl N-[4-[[2-(7-fluoro-2-methyl-indazol-5-yl)-4-methyl-pyrimidine-5-carbonyl]amino]norbornan-1-yl]carbamate (200 mg, 404.40 umol, 1 eq) in N,N-dimethylformamide (2 mL) was added N,N-dimethylformamide dimethylacetal (2 mL). The reaction mixture was stirred at 120° C. for 4 h. The reaction mixture was concentrated under reduced pressure to give a residue. To the residue was added acetic acid (2.00 mL) and the reaction mixture was stirred at 65° C. for 4 h, then filtered. The filter cake was washed with methyl tert-butyl ether (3×1 mL), and dried under vacuum to give tert-butyl N-[4-[2-(7-fluoro-2-methyl-indazol-5-yl)-5-oxo-pyrido[4,3-d]pyrimidin-6-yl]norbornan-1-yl]carbamate (120 mg, 64%) as a solid. LCMS: (ES, m/z): 505.2 [M+H]⁺. ¹HNMR (400 MHz, DMSO-d6) δ ppm 9.48 (s, 1H), 8.84 (s, 1H), 8.71 (d, J=2.50 Hz, 1H), 8.05 (d, J=13.38 Hz, 1H), 7.96 (d, J=7.75 Hz, 1H), 7.15 (br s, 1H), 6.62 (d, J=7.63 Hz, 1H), 4.24 (s, 3H), 2.36 (br s, 2H), 2.06 (br d, J=2.25 Hz, 2H), 1.91 (s, 2H), 1.72-1.85 (m, 4H), 1.41 (s, 9H).

### Synthesis of Compound 1489

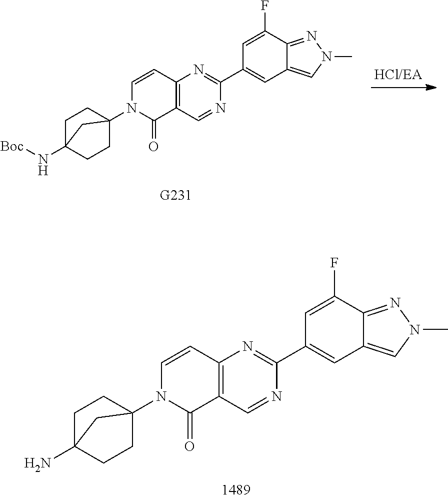 [SMILES: Cn1cc2cc(-c3ncc4c(=O)n(C56CCC(NC(=O)OC(C)(C)C)(CC5)C6)ccc4n3)cc(F)c2n1 ; Cn1cc2cc(-c3ncc4c(=O)n(C56CCC(N)(CC5)C6)ccc4n3)cc(F)c2n1]

To a solution of tert-butyl N-[4-[2-(7-fluoro-2-methyl-indazol-5-yl)-5-oxo-pyrido[4,3-d]pyrimidin-6-yl]norbornan-1-yl]carbamate (120 mg, 237.83 umol, 1 eq) in ethyl acetate (1.2 mL) was added HCl/ethyl acetate (4 M, 1.2 mL, 20.18 eq). The reaction mixture was stirred at 25° C. for 1 h, then concentrated under reduced pressure to give a residue. The residue was triturated with dichloromethane to give a suspension. The suspension was filtered and the filter cake was washed with dichloromethane, then dried under vacuum to give 6-(4-aminonorbornane-1-yl)-2-(7-fluoro-2-methyl-indazol-5-yl)pyrido[4,3-d]pyrimidin-5-one (90 mg, 92%). LCMS: (ES, m/z): 405.2 [M+H]⁺. ¹HNMR (400 MHz, DMSO-d6) δ ppm 9.47 (s, 1H), 8.81 (s, 1H), 8.67 (d, J=2.63 Hz, 1H), 8.03 (d, J=13.26 Hz, 1H), 7.90 (d, J=7.88 Hz, 1H), 6.68 (d, J=7.88 Hz, 1H), 4.21 (s, 3H), 2.56 (br s, 2H), 2.40 (s, 2H), 1.98 (br d, J=6.63 Hz, 4H), 1.81-1.90 (m, 2H).

### Example G118: Synthesis of Compound 1490

### Synthesis of Intermediate G232

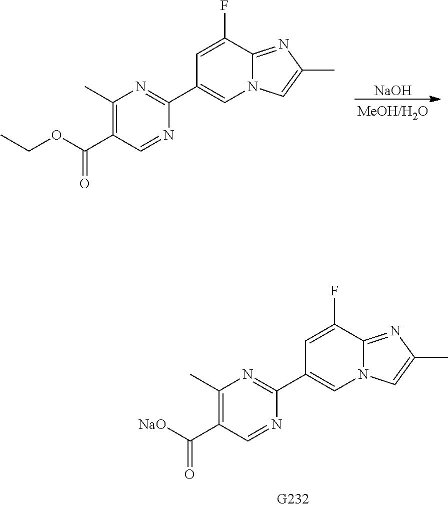

To a solution of ethyl 2-(8-fluoro-2-methyl-imidazo[1,2-a]pyridin-6-yl)-4-methyl-pyrimidine-5-carboxylate (1.20 g, 3.82 mmol, 1 eq) in methanol (25.0 mL) and water (12.0 mL) was added the solution of NaOH (610 mg, 15.2 mmol, 4 eq) in water (12.0 mL). The reaction mixture was stirred at 25° C. for 12 h, then concentrated under reduced pressure to give [2-(8-fluoro-2-methyl-imidazo[1,2-a]pyridin-6-yl)-4-methyl-pyrimidine-5-carbonyl]oxysodium (1.00 g, 85%) as a solid. LCMS: (ES, m/z): 287.1 [M+H]⁺. ¹H NMR (400 MHz, METHANOL-d₄) 6 ppm 9.26 (d, J=1.19 Hz, 1H), 8.87 (s, 1H), 7.98 (dd, J=12.16, 1.19 Hz, 1H), 7.81 (d, J=2.26 Hz, 1H), 2.77 (s, 3H), 2.44 (s, 3H). ¹⁹F NMR (376 MHz, METHANOL-d₄) δ ppm−134.379.

### Synthesis of Intermediate G233

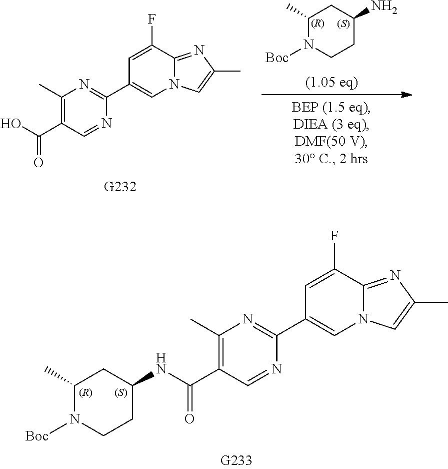

A mixture of [2-(8-fluoro-2-methyl-imidazo[1,2-a]pyridin-6-yl)-4-methyl-pyrimidine-5-carbonyl]oxysodium (220 mg, 713 umol, 1 eq), tert-butyl (2R,4 S)-4-amino-2-methyl-piperidine-1-carboxylate (168 mg, 785 umol, 1.05 eq), 2-bromo-1-ethyl-pyridin-1-ium; tetrafluoroborate (234 mg, 856 umol, 1.5 eq), and N-ethyl-N-propan-2-ylpropan-2-amine (230 mg, 1.78 mmol, 310 uL, 3.0 eq) in DMF (12.5 mL) was degassed and purged with N₂ 3 times, then stirred at 30° C. for 2 h under N₂ atmosphere. The reaction mixture was quenched with water (10.0 mL) at 0° C., diluted with ethyl acetate (10.0 mL), and the layers separated. The aqueous layer was extracted with ethyl acetate (3×10.0 mL). The organic layers were combined, washed with brine (3×10.0 mL), dried over Na₂SO₄, filtered, and concentrated under reduced pressure to give a residue. The residue was purified by column chromatography (SiO₂, Petroleum ether/Ethyl acetate=5/1 to 0/1) to give tert-butyl (2R,4S)-4-[[2-(8-fluoro-2-methyl-imidazo[1,2-a]pyridin-6-yl)-4-methyl-pyrimidine-5-carbonyl]amino]-2-methyl-piperidine-1-carboxylate (190 mg, 55%) as a solid. LCMS: (ES, m/z): 483.6 [M+H]^{+ 1}H NMR (400 MHz, CHLOROFORM-d) δ ppm 8.78 (s, 1H), 8.02 (s, 1H), 7.71 (br d, J=8.34 Hz, 1H), 7.52 (br d, J=2.03 Hz, 2H), 4.80 (br s, 1H), 2.96 (s, 3H), 2.78-2.92 (m, 5H), 2.52 (s, 3H), 2.19 (br s, 1H), 1.79-1.98 (m, 4H), 1.48 (s, 9H).

### Synthesis of Intermediate G234

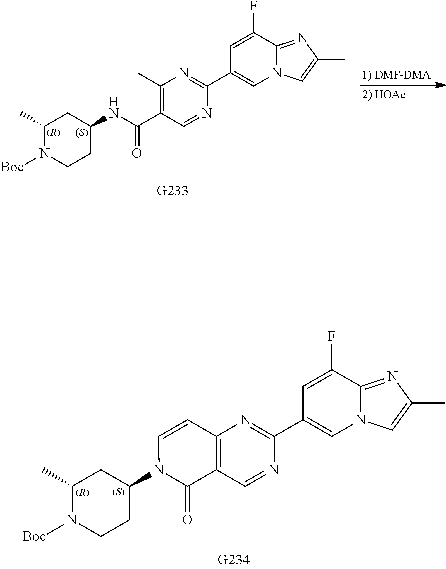

To a solution of tert-butyl (2R,4S)-4-[[2-(8-fluoro-2-methyl-imidazo[1,2-a]pyridin-6-yl)-4-methyl-pyrimidine-5-carbonyl]amino]-2-methyl-piperidine-1-carboxylate (140 mg, 290 umol, 1 eq) in DMF (500 uL) was added 1,1-dimethoxy-N,N-dimethylmethanamine (1.26 g, 1.40 mL). The reaction mixture was stirred at 130° C. for 1 h, then concentrated under reduced pressure to give a residue. To the residue was added acetic acid (2.00 g, 1.90 mL) and the reaction mixture was stirred at 65° C. for 2 h, then concentrated under reduced pressure to give a residue. The residue was triturated with methyl tert-butyl ether (2.00 mL) at 25° C. for 30 min to give a suspension. The suspension was filtered and the filter cake dried to give tert-butyl (2R,4S)-4-[2-(8-fluoro-2-methyl-imidazo[1,2-a]pyridin-6-yl)-5-oxo-pyrido[4,3-d]pyrimidin-6-yl]-2-methyl-piperidine-1-carboxylate (100 mg, 28%) as a solid. LCMS: (ES, m/z): 493.6 [M+H]⁺. ¹H NMR (400 MHz, DMSO-d₆) δ ppm 9.52-9.55 (m, 2H), 8.15 (d, J=6.90 Hz, 1H), 8.06 (d, J=3.26 Hz, 1H), 7.93 (d, J=12.17 Hz, 1H), 6.71 (d, J=7.53 Hz, 1H), 5.08-5.26 (m, 1H), 3.96-4.15 (m, 1H), 2.39 (s, 4H), 2.35 (s, 3H), 1.66-1.75 (m, 2H), 1.43 (s, 9H), 1.21-1.25 (m, 3H). ¹⁹F NMR (376 MHz, DMSO-d₆) δ ppm−131.933.

### Synthesis of Compound 1490

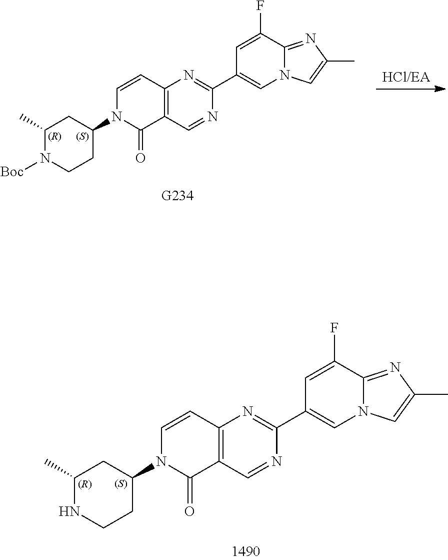

To a solution of tert-butyl (2R,4S)-4-[2-(8-fluoro-2-methyl-imidazo[1,2-a]pyridin-6-yl)-5-oxo-pyrido[4,3-d]pyrimidin-6-yl]-2-methyl-piperidine-1-carboxylate (100 mg, 203 umol, 1 eq) in ethyl acetate (1.00 mL) was added HCl/ethyl acetate (4 M, 1.00 mL). The reaction mixture was stirred at 20° C. for 3 h, then concentrated under reduced pressure to give a residue. The residue was triturated with dichloromethane at 20° C. for 30 min to give a suspension. The suspension was filtered and the filter cake dried to give 2-(8-fluoro-2-methyl-imidazo[1,2-a]pyridin-6-yl)-6-[(2R,4S)-2-methyl-4-piperidyl]pyrido[4,3-d]pyrimidin-5-one (65.0 mg, 81%) as a solid. LCMS: (ES, m/z): 393.3 [M+H]⁺. ¹H NMR (400 MHz, METHANOL-d₄) δ ppm 9.77 (s, 1H), 9.69 (s, 1H), 8.75 (d, J=11.04 Hz, 1H), 8.22 (s, 1H), 7.98 (d, J=7.78 Hz, 1H), 6.90 (d, J=7.78 Hz, 1H), 5.29 (tt, J=11.98, 4.02 Hz, 1H), 3.95-4.08 (m, 1H), 3.42-3.53 (m, 2H), 2.62 (s, 3H), 2.27-2.53 (m, 2H), 2.19 (br d, J=14.18 Hz, 1H), 2.01-2.11 (m, 1H), 1.58 (d, J=7.15 Hz, 3H). ¹⁹F NMR (376 MHz, METHANOL-d₄) δ ppm−134.039.

### Example G119: Synthesis of Compound 1491

### Synthesis of Intermediate G235

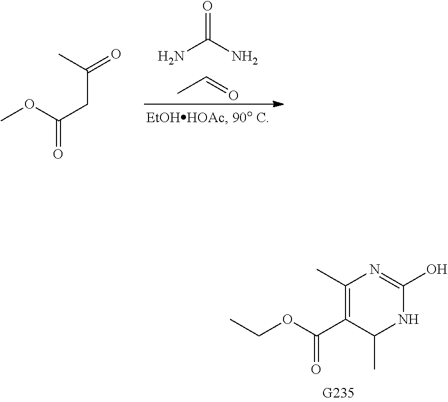

A mixture of ethyl 3-oxobutanoate (60.0 g, 461 mmol, 1 eq), acetaldehyde (27.7 g, 461 mmol, 40%, 1 eq), urea (20.3 g, 461 mmol, 1 eq), and acetic acid (2.77 g, 46.1 mmol, 0.1 eq) in ethanol (360 mL) was degassed and purged with N₂ 3 times. The reaction mixture was stirred at 90° C. for 16 h under N₂ atmosphere, then quenched with water (500 mL) at 0° C. The mixture was filtered and the filter cake was dried to give a residue. The residue was triturated with ethanol (300 mL) to give a suspension. The suspension was filtered and the filter cake dried to give ethyl 2-hydroxy-4,6-dimethyl-1,6-dihydropyrimidine-5-carboxylate (50.0 g, 55%) as a solid. LCMS: (ES, m/z): 199.4 [M+H]⁺. ¹H NMR (400 MHz, CHLOROFORM-d) δ ppm 8.04 (br s, 1H) 5.71 (br s, 1H) 4.39-4.48 (m, 1H) 4.11-4.28 (m, 2H) 2.29 (s, 3H) 1.27-1.31 (m, 6H).

### Synthesis of Intermediate G236

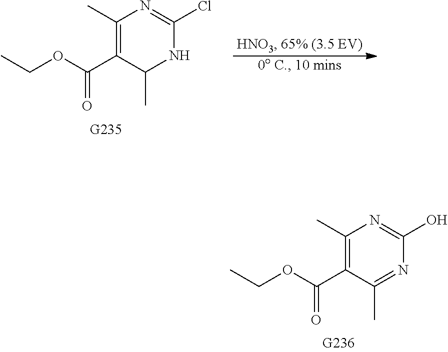

A mixture of ethyl 2-hydroxy-4,6-dimethyl-1,6-dihydropyrimidine-5-carboxylate (30.0 g, 151.3 mmol, 1 eq) in HNO₃ (105 mL) was degassed and purged with N₂ 3 times, then stirred at 0° C. for 10 min under N₂ atmosphere. The reaction mixture was quenched with water (500 mL) slowly at 0° C., then adjusted to pH 7 with solid K₂CO₃ and extracted with dichloromethane (10×100 mL). The organic layers were combined, dried over Na₂SO₄, filtered, and the filtrate concentrated under reduced pressure to give a residue. The residue was triturated with tert-butyl methyl ether (200 mL) to give a suspension. The suspension was filtered and the filter cake was dried to give ethyl 2-hydroxy-4,6-dimethyl-pyrimidine-5-carboxylate (25.0 g, 84%) as a solid. LCMS: (ES, m/z): 197.7 [M+H]⁺. ¹H NMR (400 MHz, DMSO-d₆) δ ppm 4.26 (q, J=7.13 Hz, 2H) 2.35 (s, 6H) 1.28 (t, J=7.07 Hz, 3H).

### Synthesis of Intermediate G237

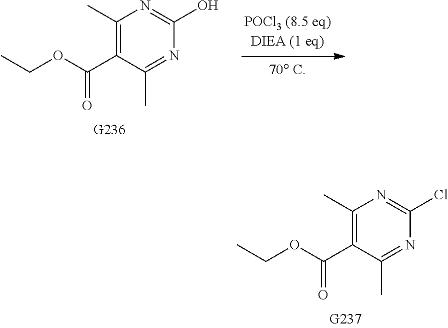

To a solution of ethyl 2-hydroxy-4,6-dimethyl-pyrimidine-5-carboxylate (20.0 g, 102 mmol, 1 eq) in POCl₃ (135 g, 866 mmol, 8.5 eq) was added N,N-diisopropylethylamine (13.2 g, 102 mmol, 1 eq) at 25° C. The reaction mixture was stirred at 70° C. for 2 h, then cooled to 25° C., poured into water (1.00 L), and extracted with ethyl acetate (3×1.00 L). The organic layers were combined, washed with brine (1.00 L), dried with anhydrous Na₂SO₄, filtered, and the filtrate concentrated to give a residue. The residue was purified by column chromatography on silica gel (SiO₂, 1.5% of ethyl acetate in petroleum ether) to give ethyl 2-chloro-4,6-dimethyl-pyrimidine-5-carboxylate (18.0 g, 82%) as a solid. LCMS: (ES, m/z): 215.2, 217.1 [M+H]⁺. 1H NMR (400 MHz, METHANOL-d₄) δ ppm 4.45 (q, J=7.15 Hz, 2H) 2.52 (s, 6H) 1.40 (t, J=7.15 Hz, 3H).

### Synthesis of Intermediate G238

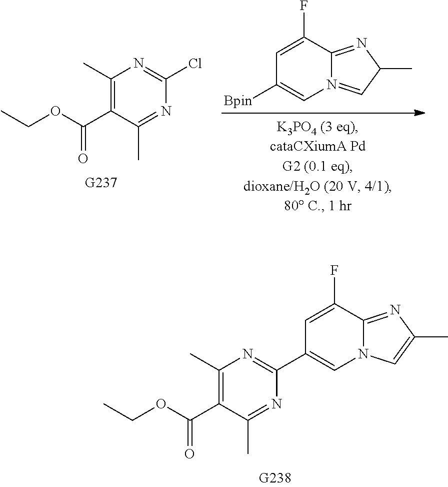

A mixture of ethyl 2-chloro-4,6-dimethyl-pyrimidine-5-carboxylate (1.00 g, 4.94 mmol, 1 eq), 8-fluoro-2-methyl-6-(4,4,5,5-tetramethyl-1,3,2-dioxaborolan-2-yl)imidazo[1,2-a]pyridine (1.5 g, 5.43 mmol, 1.1 eq), K₃PO₄ (3.15 g, 14.8 mmol, 3 eq), and [2-(2-aminophenyl)phenyl]-chloro-palladium; bis(1-adamantyl)-butyl-phosphane (330 mg, 494 umol, 0.1 eq) in dioxane (16.0 mL) and water (4.0 mL) was degassed and purged with N₂ 3 times, then stirred at 80° C. for 1 h under N₂ atmosphere and cooled to 25° C. The reaction mixture was filtered through a celite pad and washed with dichloromethane (3×30.0 mL). The filtrate was concentrated to give a residue, which was purified by column chromatography on silica gel (eluting with a gradient of 37% ethyl acetate in petroleum ether over 40 mins) to give ethyl 2-(8-fluoro-2-methyl-imidazo[1,2-a]pyridin-6-yl)-4,6-dimethyl-pyrimidine-5-carboxylate (1.00 g, 62%) as a solid. LCMS: (ES, m/z): 329.6 [M+H]⁺. ¹H NMR (400 MHz, CHLOROFORM-d) δ ppm 9.09 (d, J=1.10 Hz, 1H) 7.95 (dd, J=11.74, 1.10 Hz, 1H) 7.49 (d, J=2.32 Hz, 1H) 4.46 (q, J=7.21 Hz, 2H) 2.61 (s, 6H) 2.50 (s, 3H) 1.43 (t, J=7.15 Hz, 3H).

### Synthesis of Intermediate G239

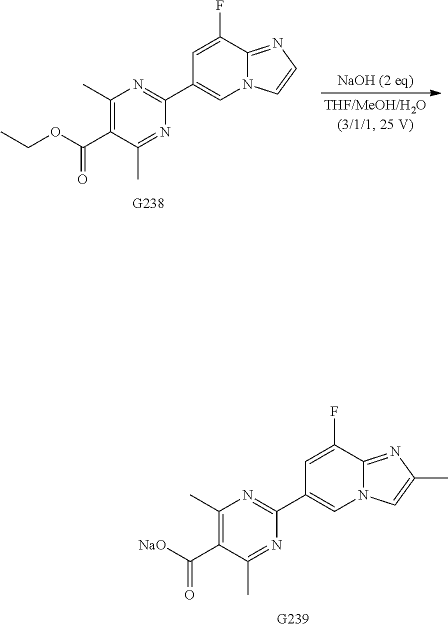

To the mixture of ethyl 2-(8-fluoro-2-methyl-imidazo[1,2-a]pyridin-6-yl)-4,6-dimethyl-pyrimidine-5-carboxylate (1.00 g, 3.05 mmol, 1 eq) in a mixture of tetrahydrofuran (15.0 mL), methanol (5.00 mL) and water (5.00 mL) was added NaOH (243 mg, 6.09 mmol, 2 eq). The reaction mixture was stirred at 25° C. for 16 h, then filtered and the filtrate was concentrated under reduced pressure to give a residue. The residue was triturated with dichloromethane (20.0 mL) to give a suspension. The suspension was filtered and the filter cake was dried to give [2-(8-fluoro-2-methyl-imidazo[1,2-a]pyridin-6-yl)-4,6-dimethyl-pyrimidine-5-carbonyl]oxysodium (800 mg, 76%) as a solid. LCMS: (ES, m/z): 301.6 [M+H]⁺. ¹H NMR (400 MHz, METHANOL-d₄) δ ppm 9.24 (d, J=1.13 Hz, 1H) 7.98 (dd, J=12.23, 1.07 Hz, 1H) 7.81 (d, J=2.26 Hz, 1H) 2.58 (s, 6H) 2.45 (s, 3H).

### Synthesis of Intermediate G240

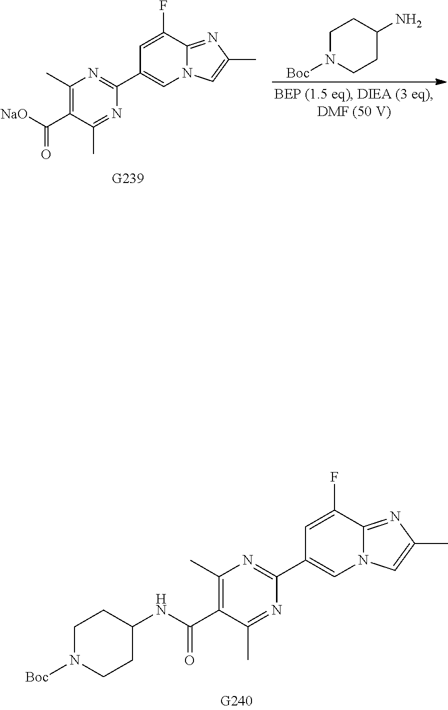

To a mixture of [2-(8-fluoro-2-methyl-imidazo[1,2-a]pyridin-6-yl)-4,6-dimethyl-pyrimidine-5-carbonyl]oxysodium (600 mg, 1.86 mmol, 1 eq), tert-butyl 4-aminopiperidine-1-carboxylate (447 mg, 2.23 mmol, 1 eq), and 2-bromo-1-ethyl-pyridin-1-ium; tetrafluoroborate (765 mg, 2.79 mmol, 1.5 eq) in N,N-dimethylformamide (30.0 mL) was added N,N-diisopropylethylamine (722 mg, 5.59 mmol, 3 eq). The reaction mixture was degassed and purged with N₂ 3 times, then stirred at 30° C. for 2 h under N₂ atmosphere. The reaction mixture was quenched with water (200 mL) and extracted with ethyl acetate (3×60.0 mL). The organic layers were combined, dried over Na₂SO₄, filtered, and the filtrate was concentrated under reduced pressure to give a residue, which was purified by column chromatography on silica gel (SiO₂, 71% of ethyl acetate in petroleum ether) to give tert-butyl 4-[[2-(8-fluoro-2-methyl-imidazo[1,2-a]pyridin-6-yl)-4,6-dimethyl-pyrimidine-5-carbonyl]amino]piperidine-1-carboxylate (500 mg, 56%) as an oil. LCMS: (ES, m/z): 483.3 [M−55+H]⁺. ¹H NMR (400 MHz, METHANOL-d₄) δ ppm 9.29 (d, J=1.13 Hz, 1H) 7.98 (dd, J=12.07, 1.06 Hz, 1H) 7.81 (d, J=2.13 Hz, 1H) 4.11-4.19 (m, 1H) 2.82-3.11 (m, 4H) 2.54 (s, 6H) 2.44 (s, 3H) 1.99-2.06 (m, 2H) 1.83 (br dd, J=12.82, 3.31 Hz, 2H) 1.47 (s, 9H).

### Synthesis of Intermediate G241

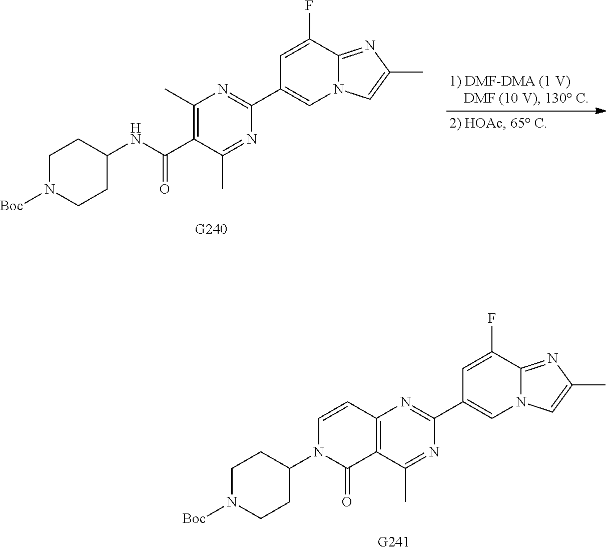 [SMILES: Cc1cn2cc(-c3nc(C)c(C(=O)NC4CCN(C(=O)OC(C)(C)C)CC4)c(C)n3)cc(F)c2n1 ; Cc1cn2cc(-c3nc(C)c4c(=O)n(C5CCN(C(=O)OC(C)(C)C)CC5)ccc4n3)cc(F)c2n1]

To a solution of tert-butyl 4-[[2-(8-fluoro-2-methyl-imidazo[1,2-a]pyridin-6-yl)-4,6-dimethyl-pyrimidine-5-carbonyl]amino]piperidine-1-carboxylate (500 mg, 1.04 mmol, 1 eq) in N,N-dimethylformamide (5.00 mL) was added N,N-dimethylformamide-dimethylacetamide (500 uL). The resulting mixture was stirred at 130° C. for 2 h, then concentrated under reduced pressure to give a residue. To the residue was added acetic acid (5.00 mL). The resulting mixture was degassed and purged with N₂ 3 times, then stirred at 65° C. for 2 h under N₂ atmosphere. The reaction mixture was concentrated under reduced pressure to give a residue, which was purified by column chromatography on silica gel (SiO₂, 50% of ethyl acetate in petroleum ether) to give tert-butyl 4-[2-(8-fluoro-2-methyl-imidazo[1,2-a]pyridin-6-yl)-4-methyl-5-oxo-pyrido[4,3-d]pyrimidin-6-yl]piperidine-1-carboxylate (200 mg, 44%) as an oil. LCMS: (ES, m/z): 493.3 [M+H]⁺. ¹H NMR (400 MHz, METHANOL-d₄) δ ppm 9.39 (d, J=1.25 Hz, 1H) 8.04 (dd, J=12.07, 1.19 Hz, 1H) 7.90 (d, J=7.63 Hz, 1H) 7.83 (d, J=2.25 Hz, 1H) 6.72 (d, J=7.63 Hz, 1H) 5.00 (dt, J=10.69, 5.41 Hz, 1H) 4.31 (br d, J=13.63 Hz, 2H) 3.07 (s, 3H) 2.89-3.04 (m, 2H) 2.45 (s, 3H) 1.82-1.96 (m, 4H) 1.50 (s, 9H).

### Synthesis of Compound 1491

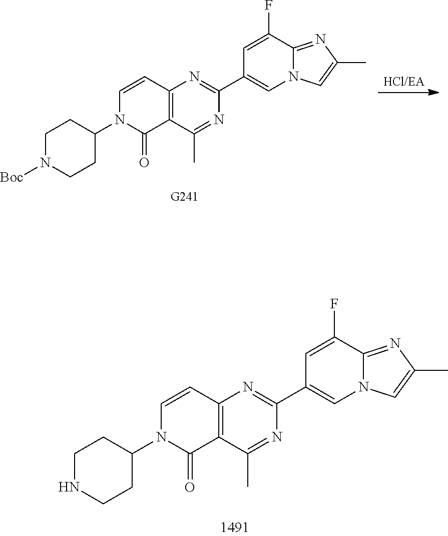 [SMILES: Cc1cn2cc(-c3nc(C)c4c(=O)n(C5CCN(C(=O)OC(C)(C)C)CC5)ccc4n3)cc(F)c2n1 ; Cc1cn2cc(-c3nc(C)c4c(=O)n(C5CCNCC5)ccc4n3)cc(F)c2n1]

To a mixture of tert-butyl 4-[2-(8-fluoro-2-methyl-imidazo[1,2-a]pyridin-6-yl)-4-methyl-5-oxo-pyrido[4,3-d]pyrimidin-6-yl]piperidine-1-carboxylate (200.0 mg, 406 umol, 1 eq) in ethyl acetate (1.00 mL) was added HCl/ethyl acetate (4.00 M, 1.00 mL). The reaction mixture was stirred at 25° C. for 2 h, then filtered and the filter cake was concentrated under reduced pressure to give a residue. The residue was triturated with dichloromethane (5.00 mL) to give a suspension. The suspension was filtered and the filter cake was dried to give 2-(8-fluoro-2-methyl-imidazo[1,2-a]pyridin-6-yl)-4-methyl-6-(4-piperidyl)pyrido[4,3-d]pyrimidin-5-one (103 mg, 65%) as a solid. LCMS: (ES, m/z): 393.2 [M+H]⁺. ¹H NMR (400 MHz, METHANOL-d₄) δ ppm 9.77 (d, J=1.00 Hz, 1H) 8.77 (dd, J=11.07, 1.06 Hz, 1H) 8.23 (d, J=1.00 Hz, 1H) 7.90 (d, J=7.75 Hz, 1H) 6.84 (d, J=7.63 Hz, 1H) 5.05-5.12 (m, 1H) 3.59-3.67 (m, 2H) 3.23-3.30 (m, 2H) 3.14 (s, 3H) 2.62 (d, J=0.88 Hz, 3H) 2.30 (qd, J=13.03, 4.06 Hz, 2H) 2.18 (br d, J=14.76 Hz, 2H) ¹⁹F NMR (376 MHz, METHANOL-d₄) δ ppm−134.236 (s, 1F).

### Example G120: Synthesis of Compound 1493

### Synthesis of Intermediate G242

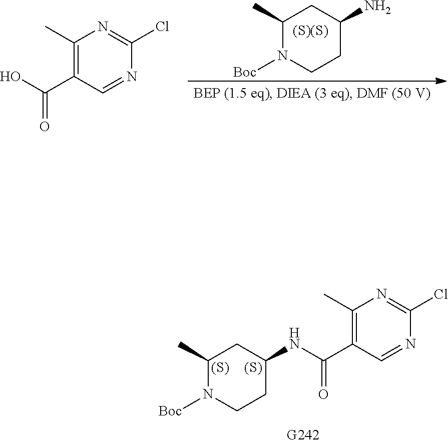

To a mixture of 2-chloro-4-methyl-pyrimidine-5-carboxylic acid (200 mg, 1.16 mmol, 1 eq), tert-butyl (2S,4S)-4-amino-2-methyl-piperidine-1-carboxylate (248 mg, 116 mmol, 1 eq), and 2-bromo-1-ethyl-pyridin-1-ium; tetrafluoroborate (476 mg, 1.74 mmol, 1.5 eq) in N,N-dimethylformamide (10.0 mL) was added N,N-diisopropylethylamine (449 mg, 348 mmol, 3 eq). The reaction mixtures was degassed and purged with N₂ 3 times, then stirred at 30° C. for 2 h under N₂ atmosphere. The reaction mixture was quenched with water (50.0 mL) and extracted with ethyl acetate (3×20.0 mL). The organic layers were combined, dried over Na₂SO₄, filtered, and the filtrate was concentrated under reduced pressure to give a residue, which was purified by column chromatography on silica gel (SiO₂, 21% of ethyl acetate in petroleum ether) to give tert-butyl (2S,4S)-4-[(2-chloro-4-methyl-pyrimidine-5-carbonyl)amino]-2-methyl-piperidine-1-carboxylate (200 mg, 52%) as an oil. LCMS: (ES, m/z): 313.1 [M−55]⁺. ¹H NMR (400 MHz, CHLOROFORM-d) δ ppm 8.55 (s, 1H) 5.94 (br d, J=6.75 Hz, 1H) 4.24-4.31 (m, 1H) 4.20 (br d, J=6.50 Hz, 1H) 3.83-3.93 (m, 1H) 3.11-3.21 (m, 1H) 2.69 (s, 3H) 2.05-2.14 (m, 2H) 1.63-1.75 (m, 2H) 1.48 (s, 9H) 1.26-1.28 (m, 3H).

### Synthesis of Intermediate G243

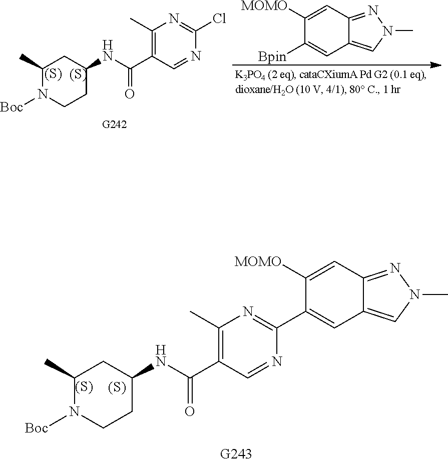

A mixture of tert-butyl (2S,4S)-4-[(2-chloro-4-methyl-pyrimidine-5-carbonyl)amino]-2-methyl-piperidine-1-carboxylate (200 mg, 542 umol, 1 eq), 6-(methoxymethoxy)-2-methyl-5-(4,4,5,5-tetramethyl-1,3,2-dioxaborolan-2-yl)indazole (360 mg, 1.13 mmol, 2 eq), K₃PO₄ (360 mg, 1.69 mmol, 3 eq), and [2-(2-aminophenyl)phenyl]-chloro-palladium; bis(1-adamantyl)-butyl-phosphane (38.2 mg, 36.5 umol, 0.1 eq) in dioxane (1.60 mL) and water (400 uL) was degassed and purged with N₂ 3 times. The reaction mixture was stirred at 80° C. for 1 h under N₂ atmosphere, then cooled to 25° C., filtered through a celite pad, and washed with dichloromethane (3×10.0 mL). The filtrate was concentrated to give a residue, which was purified by column chromatography on silica gel (eluting with a gradient of 35% ethyl acetate in petroleum ether over 40 mins) to give tert-butyl (2S,4S)-4-[[2-[6-(methoxymethoxy)-2-methyl-indazol-5-yl]-4-methyl-pyrimidine-5-carbonyl]amino]-2-methyl-piperidine-1-carboxylate (150 mg, 53%) as a solid. LCMS: (ES, m/z): 525.4[M+H]⁺, ¹H NMR (400 MHz, CHLOROFORM-d) δ ppm 8.77 (s, 1H) 7.99 (s, 1H) 7.91 (s, 1H) 7.36 (s, 1H) 5.26 (s, 2H) 4.28-4.36 (m, 1H) 4.22-4.27 (m, 1H) 4.19-4.21 (m, 3H) 3.85-3.95 (m, 1H) 3.49 (s, 3H) 3.13-3.23 (m, 1H) 2.75 (s, 3H) 2.07-2.15 (m, 2H) 1.67-1.80 (m, 2H) 1.49 (s, 9H) 1.28-1.34 (m, 3H).

### Synthesis of Intermediate G244

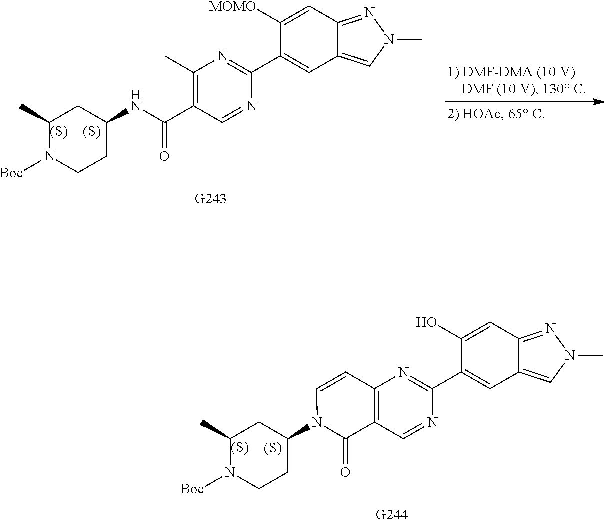

To a solution of tert-butyl (2S,4S)-4-[[2-[6-(methoxymethoxy)-2-methyl-indazol-5-yl]-4-methyl-pyrimidine-5-carbonyl]amino]-2-methyl-piperidine-1-carboxylate (150 mg, 286 umol, 1 eq) in N,N-dimethylformamide (1.50 mL) was added N,N-dimethylformamide-dimethylacetamide (1.50 mL). The resulting mixture was stirred at 130° C. for an additional 2 h, then concentrated under reduced pressure to give a residue. To the residue was added acetic acid (1.50 mL). The reaction mixture was degassed and purged with N₂ 3 times and stirred at 65° C. for 2 h under N₂ atmosphere. The reaction mixture was concentrated under reduced pressure to give a residue, which was purified by column chromatography on silica gel (SiO₂, 63% of ethyl acetate in petroleum ether) to give tert-butyl (2S,4S)-4-[2-[6-(methoxymethoxy)-2-methyl-indazol-5-yl]-5-oxo-pyrido[4,3-d]pyrimidin-6-yl]-2-methyl-piperidine-1-carboxylate (100 mg, 71%) as a solid. LCMS: (ES, m/z): 491.3 [M+H]⁺. ¹HNMR (400 MHz, CHLOROFORM-d) δ ppm 12.94-13.12 (m, 1H) 9.66 (s, 1H) 9.17 (s, 1H) 8.00 (s, 1H) 7.57 (d, J=7.78 Hz, 1H) 7.18 (s, 1H) 6.71 (d, J=7.65 Hz, 1H) 5.05 (br d, J=9.41 Hz, 1H) 4.22 (s, 3H) 3.93-4.08 (m, 1H) 3.76-3.88 (m, 1H) 3.44-3.57 (m, 1H) 2.35 (br dd, J=13.87, 8.22 Hz, 1H) 1.84-2.04 (m, 2H) 1.70-1.76 (m, 1H) 1.51 (s, 9H) 1.34 (d, J=6.27 Hz, 3H).

### Synthesis of Compound 1493

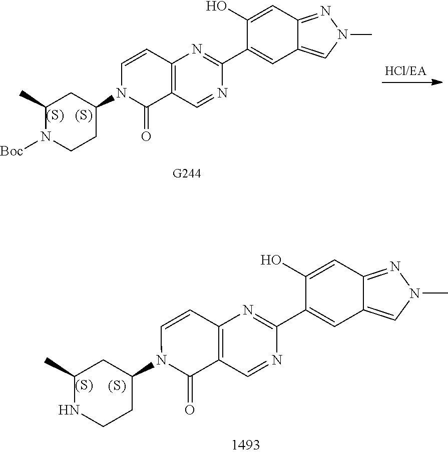

To a mixture of tert-butyl (2S,4S)-4-[2-[6-(methoxymethoxy)-2-methyl-indazol-5-yl]-5-oxo-pyrido[4,3-d]pyrimidin-6-yl]-2-methyl-piperidine-1-carboxylate (100.0 mg, 204 umol, 1 eq) in ethyl acetate (1.00 mL) was added HCl/ethyl acetate (4.00 M, 1.00 mL). The reaction mixture was stirred at 25° C. for 2 h, then filtered and the filter cake was concentrated under reduced pressure to give a residue. The residue was triturated with dichloromethane (5.00 mL) to give a suspension. The suspension was filtered and the filter cake was dried to give to give 2-(6-hydroxy-2-methyl-indazol-5-yl)-6-[(2S,4S)-2-methyl-4-piperidyl]pyrido[4,3-d]pyrimidin-5-one (19.0 mg, 24%) as a solid. LCMS: (ES, m/z): 391.2 [M+H]⁺. ¹H NMR (400 MHz, METHANOL-d₄) δ ppm 9.61 (s, 1H) 9.23 (s, 1H) 8.47 (s, 1H) 7.92 (d, J=7.78 Hz, 1H) 6.97 (s, 1H) 6.84 (d, J=7.65 Hz, 1H) 5.09 (br d, J=4.39 Hz, 1H) 4.22 (s, 3H) 3.62 (br d, J=12.92 Hz, 1H) 3.46-3.56 (m, 1H) 3.27 (br d, J=3.76 Hz, 1H) 2.17-2.33 (m, 3H) 2.10 (br d, J=12.80 Hz, 1H) 1.44 (d, J=6.53 Hz, 3H).

### Example G121: Synthesis of Compound 1496

### Synthesis of Intermediate G245

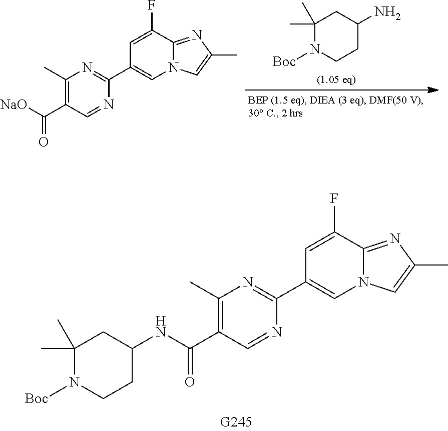

To a mixture of [2-(8-fluoro-2-methyl-imidazo[1,2-a]pyridin-6-yl)-4-methyl-pyrimidine-5-carbonyl]oxysodium (300 mg, 973 umol, 1.00 eq) and tert-butyl 4-amino-2,2-dimethyl-piperidine-1-carboxylate (233 mg, 1.02 mmol, 1.05 eq) in dimethyl formamide (15.0 mL) was added 2-bromo-1-ethyl-pyridin-1-ium; tetrafluoroborate (399 mg, 1.46 mmol, 1.50 eq) and N,N-diisopropylethylamine (377 mg, 2.92 mmol, 3.00 eq) at 25° C. The reaction mixture was stirred at 30° C. for 2 h, then poured into water (100 mL) and extracted with ethyl acetate (3×100 mL). The organic layers were combined, washed with brine (200 mL), dried over anhydrous Na₂SO₄, filtered, and the filtrate concentrated under reduced pressure to give a residue. The residue was purified by silica gel column chromatography (petroleum ether/ethyl acetate=2/1) to give tert-butyl 4-[[2-(8-fluoro-2-methyl-imidazo[1,2-a]pyridin-6-yl)-4-methyl-pyrimidine-5-carbonyl]amino]-2,2-dimethyl-piperidine-1-carboxylate (500 mg, 80%) as an oil. LCMS: (ESI, m/z): 497.4 [M+H]⁺. ¹H NMR (400 MHz, DMSO-d₆) δ ppm 9.38 (d, J=1.19 Hz, 1H) 8.75 (s, 1H) 8.01 (d, J=2.50 Hz, 1H) 7.86 (d, J=12.16 Hz, 1H) 4.04-4.12 (m, 1H) 3.69-3.78 (m, 1H) 3.56 (s, 1H) 3.15 (ddd, J=13.59, 10.37, 3.70 Hz, 1H) 2.60 (s, 3H) 2.37 (s, 3H) 1.94-2.01 (m, 1H) 1.76 (br dd, J=13.17, 3.16 Hz, 1H) 1.57 (br t, J=12.34 Hz, 1H) 1.47 (s, 3H) 1.39 (s, 9H) 1.30 (s, 3H).

### Synthesis of Intermediate G246

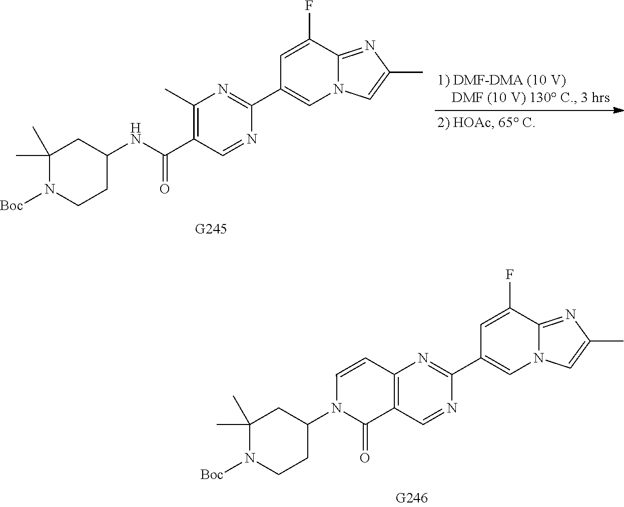

To a solution of tert-butyl 4-[[2-(8-fluoro-2-methyl-imidazo[1,2-a]pyridin-6-yl)-4-methyl-pyrimidine-5-carbonyl]amino]-2,2-dimethyl-piperidine-1-carboxylate (500 mg, 1.01 mmol, 1.00 eq) in dimethyl formamide (5.00 mL) was added 1,1-dimethoxy-N,N-dimethylmethanamine (5.00 mL) at 25° C. under N₂ atmosphere. The reaction mixture was stirred at 130° C. for 3 h, then cooled to 25° C., and concentrated under reduced pressure to give a residue. To the residue was added acetic acid (5.00 mL) and the resulting mixture was stirred at 65° C. for 6 h. The reaction mixture was concentrated under reduced pressure to give a residue, which was triturated with methyl tert-butyl ether (5.00 mL) to give a suspension. The suspension was filtered and the filter cake was dried to give tert-butyl 4-[2-(8-fluoro-2-methyl-imidazo[1,2-a]pyridin-6-yl)-5-oxo-pyrido[4,3-d]pyrimidin-6-yl]-2,2-dimethyl-piperidine-1-carboxylate (280 mg, 55%) as a solid. LCMS: (ESI, m/z): 507.4 [M+H]⁺. ¹H NMR (400 MHz, METHANOL-d₄) δ ppm 9.61 (s, 1H) 9.47 (s, 1H) 8.11 (d, J=11.92 Hz, 1H) 8.00 (s, 1H) 7.87 (br s, 1H) 6.83 (d, J=8.16 Hz, 1H) 5.17-5.22 (m, 1H) 3.98-4.09 (m, 1H) 3.47 (br d, J=9.03 Hz, 1H) 2.47 (s, 3H) 2.08-2.16 (m, 2H) 1.87-1.99 (m, 1H) 1.76 (br dd, J=13.99, 4.83 Hz, 1H) 1.60 (s, 3H) 1.50 (br s, 3H) 1.49 (s, 9H) ¹⁹F NMR (376 MHz, METHANOL-d₄) δ ppm−133.95.

### Synthesis of Intermediate G247 and G248

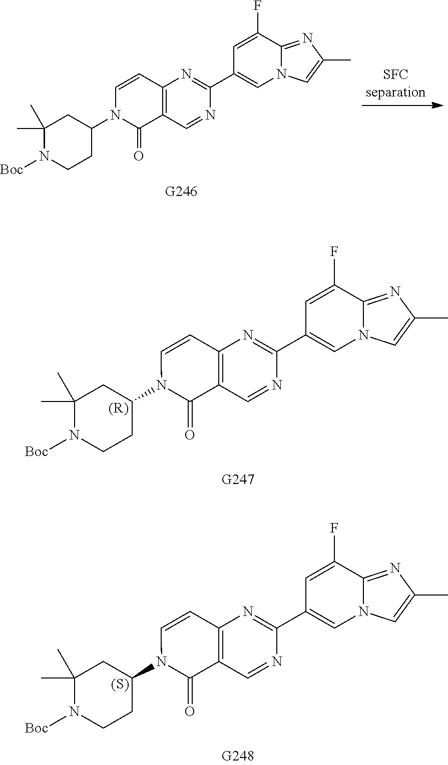

Tert-butyl 4-[2-(8-fluoro-2-methyl-imidazo[1,2-a]pyridin-6-yl)-5-oxo-pyrido[4,3-d]pyrimidin-6-yl]-2,2-dimethyl-piperidine-1-carboxylate (280 mg, 552 umol, 1.00 eq) was purified by SFC (Condition G4) to give tert-butyl (4R)-4-[2-(8-fluoro-2-methyl-imidazo[1,2-a]pyridin-6-yl)-5-oxo-pyrido[4,3-d]pyrimidin-6-yl]-2,2-dimethyl-piperidine-1-carboxylate (120 mg, 43%) and tert-butyl (4S)-4-[2-(8-fluoro-2-methyl-imidazo[1,2-a]pyridin-6-yl)-5-oxo-pyrido[4,3-d]pyrimidin-6-yl]-2,2-dimethyl-piperidine-1-carboxylate (90.0 mg, 32%) as solids. G247: LCMS: (ESI, m/z): 507.3 [M+H]⁺. ¹H NMR (400 MHz, DMSO-d₆) δ ppm 9.55 (d, J=1.38 Hz, 1H) 9.53 (s, 1H) 8.14 (d, J=7.75 Hz, 1H) 8.06 (d, J=2.25 Hz, 1H) 7.89-7.99 (m, 1H) 6.73 (d, J=7.75 Hz, 1H) 5.02-5.11 (m, 1H) 3.87-3.97 (m, 1H) 3.75-3.82 (m, 1H) 2.39 (s, 3H) 2.05 (t, J=12.88 Hz, 1H) 1.91-1.97 (m, 2H) 1.66-1.69 (m, 1H) 1.51 (s, 3H) 1.42 (s, 9H) 1.38 (s, 3H). ¹⁹F NMR (376 MHz, DMSO-d₆) δ ppm−131.93. G248: LCMS: (ESI, m/z): 507.3 [M+H]⁺. ¹H NMR (400 MHz, DMSO-d₆) δ ppm 9.51-9.58 (m, 2H) 8.14 (d, J=7.75 Hz, 1H) 8.06 (d, J=2.25 Hz, 1H) 7.93 (dd, J=12.26, 1.25 Hz, 1H) 6.73 (d, J=7.75 Hz, 1H) 5.01-5.13 (m, 1H) 3.89-3.98 (m, 1H) 3.27-3.31 (m, 1H) 2.39 (s, 3H) 2.05 (t, J=12.69 Hz, 1H) 1.88-1.99 (m, 2H) 1.67 (br dd, J=12.76, 2.50 Hz, 1H) 1.51 (s, 3H) 1.42 (s, 9H) 1.38 (s, 3H). ¹⁹F NMR (376 MHz, DMSO-d₆) δ ppm −131.93.

### Synthesis of Compound 1496

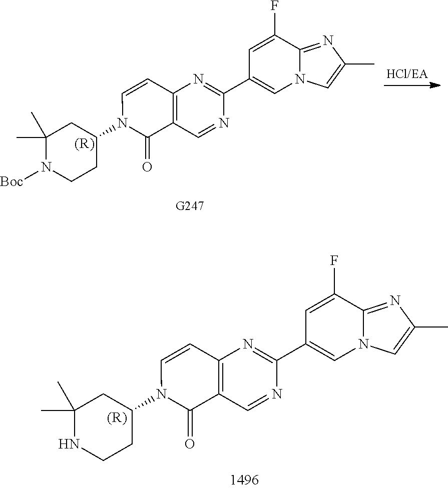

To a solution of tert-butyl (4R)-4-[2-(8-fluoro-2-methyl-imidazo[1,2-a]pyridin-6-yl)-5-oxo-pyrido[4,3-d]pyrimidin-6-yl]-2,2-dimethyl-piperidine-1-carboxylate (120 mg, 236 umol, 1.00 eq) in ethyl acetate (2.40 mL) was added HCl/ethyl acetate (4.0 M, 2.40 mL) at 25° C. The reaction mixture was stirred at 25° C. for 4 h, then concentrated to give a residue which was triturated with methyl tert-butyl ether (5.00 mL) to give a suspension. The suspension was filtered and the filter cake was dried under reduced pressure to give 6-[(4R)-2,2-dimethyl-4-piperidyl]-2-(8-fluoro-2-methyl-imidazo[1,2-a]pyridin-6-yl)pyrido[4,3-d]pyrimidin-5-one (63.2 mg, 66%) as a solid. LCMS: (ESI, m/z): 407.2 [M+H]⁺. ¹H NMR (400 MHz, METHANOL-d₄) δ ppm 9.79 (d, J=0.75 Hz, 1H) 9.69 (s, 1H) 8.76-8.82 (m, 1H) 8.25 (s, 1H) 7.99 (d, J=7.78 Hz, 1H) 6.91 (d, J=7.78 Hz, 1H) 5.22-5.36 (m, 1H) 3.47-3.57 (m, 2H) 2.63 (s, 3H) 2.31 (br dd, J=12.11, 7.34 Hz, 1H) 2.16-2.27 (m, 2H) 2.05-2.12 (m, 1H) 1.52-1.61 (m, 6H) ¹⁹F NMR (376 MHz, DMSO-d6) δ ppm−134.05.

### Synthesis of Compound 1497

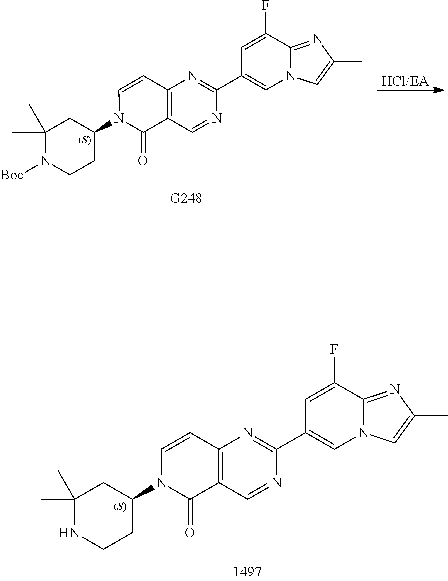

To a solution of tert-butyl (4S)-4-[2-(8-fluoro-2-methyl-imidazo[1,2-a]pyridin-6-yl)-5-oxo-pyrido[4,3-d]pyrimidin-6-yl]-2,2-dimethyl-piperidine-1-carboxylate (90.0 mg, 177 umol, 1.00 eq) in ethyl acetate (1.80 mL) was added HCl/ethyl acetate (4.0 M, 1.80 mL) at 25° C. The reaction mixture was stirred at 25° C. for 4 h, then concentrated to give a residue which was triturated with methyl tert-butyl ether (5.00 mL) to give a suspension. The suspension was filtered and the filter cake was dried under reduced pressure to give 6-[(4S)-2,2-dimethyl-4-piperidyl]-2-(8-fluoro-2-methyl-imidazo[1,2-a]pyridin-6-yl)pyrido[4,3-d]pyrimidin-5-one (53.4 mg, 74%) as a solid. LCMS: (ESI, m/z): 407.2 [M+H]⁺. ¹H NMR (400 MHz, METHANOL-d₄) δ ppm 9.78 (s, 1H) 9.69 (s, 1H) 8.76 (d, J=11.04 Hz, 1H) 8.23 (d, J=0.63 Hz, 1H) 8.00 (d, J=7.78 Hz, 1H) 6.91 (d, J=7.65 Hz, 1H) 5.29 (br t, J=4.02 Hz, 1H) 3.43-3.53 (m, 2H) 2.62 (s, 3H) 2.28-2.40 (m, 1H) 2.16-2.27 (m, 2H) 2.08 (br dd, J=13.43, 1.88 Hz, 1H) 1.56 (d, J=18.82 Hz, 6H). ¹⁹F NMR (376 MHz, METHANOL-d₄) δ ppm−134.03.

### Example G122: Synthesis of Compound 1498

### Synthesis of Intermediate G249

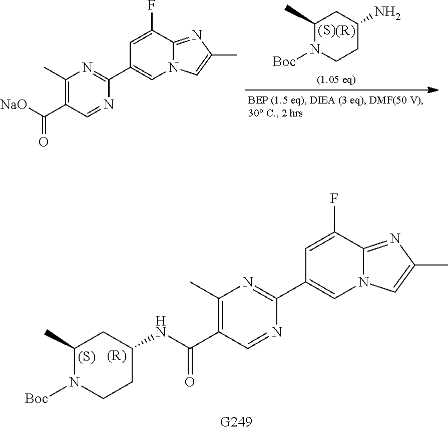

To a mixture of 2-(8-fluoro-2-methyl-imidazo[1,2-a]pyridin-6-yl)-4-methyl-pyrimidine-5-carboxylic acid (200 mg, 646.72 umol, 1 eq, Na) and tert-butyl (2S,4R)-4-amino-2-methyl-piperidine-1-carboxylate (145.53 mg, 679.06 umol, 1.05 eq) in N,N-dimethylformamide (10 mL) was added 2-bromo-1-ethyl-pyridin-1-ium tetrafluoroborate (265.67 mg, 970.09 umol, 1.5 eq) and N,N-diisopropylethylamine (250.75 mg, 1.94 mmol, 337.94 uL, 3 eq). The reaction mixture was stirred at 30° C. for 2 h, then diluted with water (20 mL) and extracted with ethyl acetate (30 mL×3). The organic layers were combined, washed with brine 100 mL, dried over anhydrous sodium sulfate, filtered, and the filtrate concentrated under reduced pressure to give a residue. The residue was triturated with dichloromethane at 20° C. for 5 min to give a suspension. The suspension was filtered. The filter cake was washed with dichloromethane and dried in vacuum to give tert-butyl (2S,4R)-4-[[2-(8-fluoro-2-methyl-imidazo[1,2-a]pyridin-6-yl)-4-methyl-pyrimidine-5-carbonyl]amino]-2-methyl-piperidine-1-carboxylate (140 mg, 45%) as a solid. LCMS: (ES, m/z): 483.2 [M+H]⁺. ¹HNMR (400 MHz, DMSO-d6) δ ppm 9.41 (d, J=1.25 Hz, 1H), 8.77 (s, 1H), 8.54 (d, J=7.63 Hz, 1H), 8.03 (d, J=2.38 Hz, 1H), 7.81-7.90 (m, 1H), 4.38 (br s, 1H), 4.07-4.23 (m, 1H), 3.81-3.98 (m, 1H), 2.96 (br s, 1H), 2.61 (s, 3H), 2.38 (s, 3H), 1.91 (br d, J=12.13 Hz, 1H), 1.74-1.83 (m, 1H), 1.52 (td, J=12.57, 5.63 Hz, 1H), 1.40 (s, 9H), 1.23-1.34 (m, 1H), 1.17 (d, J=6.88 Hz, 3H).

### Synthesis of Intermediate G250

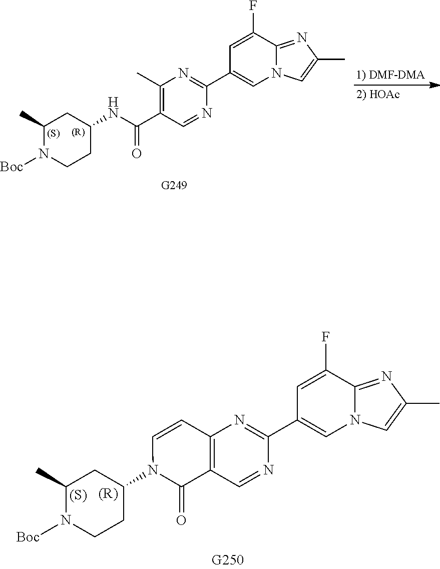

To a solution of tert-butyl (2S,4R)-4-[[2-(8-fluoro-2-methyl-imidazo[1,2-a]pyridin-6-yl)-4-methyl-pyrimidine-5-carbonyl]amino]-2-methyl-piperidine-1-carboxylate (140 mg, 290.13 umol, 1 eq) in N,N-dimethylformamide (1.4 mL) was added N,N-dimethylformamide dimethylacetal (1.4 mL). The reaction mixture was stirred at 130° C. for 1 h, then concentrated under reduced pressure to give a residue. To the residue was added acetic acid (2.00 mL). The reaction mixture was stirred at 65° C. for 4 h, then filtered. The filter cake was washed with methyl tert-butyl ether (3×1 mL) and dried under vacuum to give tert-butyl (2S,4R)-4-[2-(8-fluoro-2-methyl-imidazo[1,2-a]pyridin-6-yl)-5-oxo-pyrido[4,3-d]pyrimidin-6-yl]-2-methyl-piperidine-1-carboxylate (75 mg, 58%) as a solid. LCMS: (ES, m/z): 493.2 [M+H]⁺. ¹HNMR (400 MHz, DMSO-d6) δ ppm 9.49-9.56 (m, 2H), 8.15 (d, J=7.75 Hz, 1H), 8.06 (d, J=2.50 Hz, 1H), 7.93 (dd, J=12.38, 1.13 Hz, 1H), 6.71 (d, J=7.75 Hz, 1H), 5.09-5.24 (m, 1H), 4.38-4.61 (m, 1H), 4.04 (br s, 1H), 2.98-3.13 (m, 1H), 2.39 (s, 3H), 1.95-2.07 (m, 1H), 1.77-1.89 (m, 2H), 1.70 (br d, J=12.76 Hz, 1H), 1.43 (s, 9H), 1.19-1.27 (m, 3H).

### Synthesis of Compound 1498

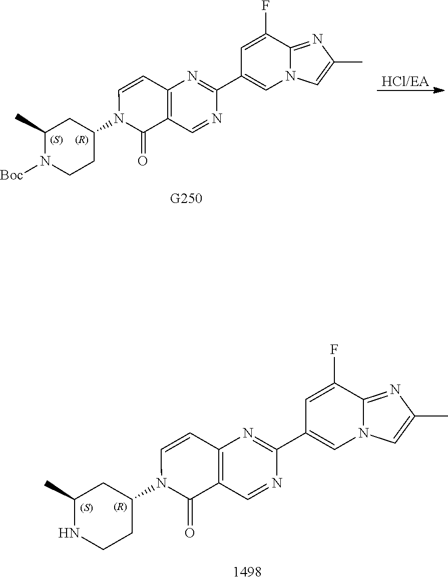

To a solution of tert-butyl (2S,4R)-4-[2-(8-fluoro-2-methyl-imidazo[1,2-a]pyridin-6-yl)-5-oxo-pyrido[4,3-d]pyrimidin-6-yl]-2-methyl-piperidine-1-carboxylate (75 mg, 152.27 umol, 1 eq) in ethyl acetate (0.8 mL) was added HCl/ethyl acetate (4 M, 0.8 mL, 21.02 eq). The reaction mixture was stirred at 25° C. for 1 h, then filtered and the filter cake was collected with water (10 mL). The aqueous solution was lyophilized to give 2-(8-fluoro-2-methyl-imidazo[1,2-a]pyridin-6-yl)-6-[(2S,4R)-2-methyl-4-piperidyl]pyrido[4,3-d]pyrimidin-5-one (63 mg, 96%) as a solid. LCMS: (ES, m/z): 393.2 [M+H]⁺. ¹HNMR (400 MHz, D₂O) δ ppm 9.61 (s, 1H), 9.44 (s, 1H), 8.51 (br d, J=10.64 Hz, 1H), 8.05 (s, 1H), 7.97 (d, J=7.82 Hz, 1H), 6.95 (d, J=7.70 Hz, 1H), 5.25-5.34 (m, 1H), 4.07 (br s, 1H), 3.43-3.57 (m, 2H), 2.55 (s, 3H), 2.36 (td, J=13.08, 4.77 Hz, 1H), 2.23 (br d, J=4.16 Hz, 2H), 2.09 (br d, J=13.69 Hz, 1H), 1.54 (br d, J=6.97 Hz, 3H).

### Example G123: Synthesis of Compound 1499

### Synthesis Intermediate G251

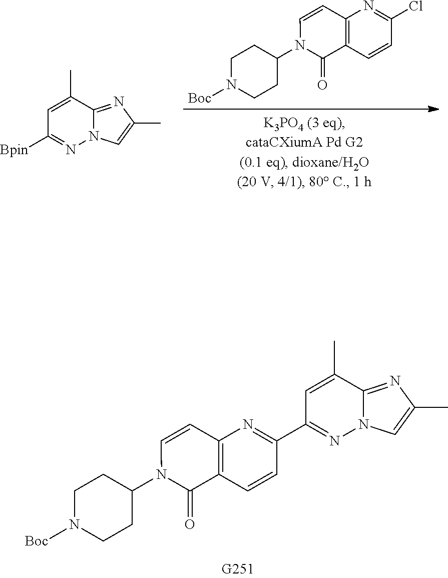

To a mixture of tert-butyl 4-(2-chloro-5-oxo-1,6-naphthyridin-6-yl)piperidine-1-carboxylate (200 mg, 549.70 umol, 1 eq) and 2,8-dimethyl-6-(4,4,5,5-tetramethyl-1,3,2-dioxaborolan-2-yl)imidazo[1,2-b]pyridazine (900.86 mg, 3.30 mmol, 6 eq) in dioxane (3.2 mL) and water (0.8 mL) was added [2-(2-aminophenyl)phenyl]-chloro-palladium; bis(1-adamantyl)-butyl-phosphane (36.75 mg, 54.97 umol, 0.1 eq) and potassium phosphate (233.37 mg, 1.10 mmol, 2 eq). The reaction mixture was stirred at 80° C. for 1 h under nitrogen, then diluted with water (10 mL) and extracted with ethyl acetate (20 mL×3). The organic layers were combined, washed with brine (60 mL), dried over anhydrous sodium sulfate, filtered, and the filtrate concentrated under reduced pressure to give a residue. The residue was purified by prep-HPLC (Condition G1, Gradient 6) to give tert-butyl 4-[2-(2,8-dimethylimidazo[1,2-b]pyridazin-6-yl)-5-oxo-1,6-naphthyridin-6-yl]piperidine-1-carboxylate (85 mg, 32%) as a solid. LCMS: (ES, m/z): 475.2 [M+H]^{+ 1}HNMR (400 MHz, DMSO-d6) δ ppm 8.72 (d, J=8.38 Hz, 1H), 8.37 (d, J=8.38 Hz, 1H), 8.16 (d, J=0.63 Hz, 1H), 8.07 (d, J=1.00 Hz, 1H), 7.92 (d, J=7.88 Hz, 1H), 6.82 (d, J=7.75 Hz, 1H), 4.90-5.04 (m, 1H), 4.14 (br d, J=9.01 Hz, 2H), 2.82-3.04 (m, 2H), 2.66 (d, J=0.75 Hz, 3H), 2.43 (s, 3H), 1.75-1.92 (m, 4H), 1.44 (s, 9H).

### Synthesis of Compound 1499

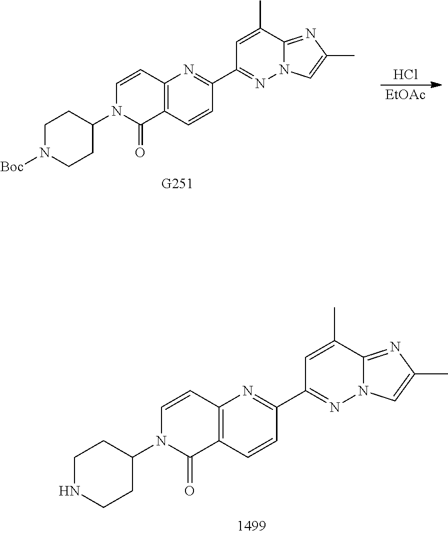 [SMILES: "EtOAc" ; Cc1cn2nc(-c3ccc4c(=O)n(C5CCN(C(=O)OC(C)(C)C)CC5)ccc4n3)cc(C)c2n1 ; Cl ; Cc1cn2nc(-c3ccc4c(=O)n(C5CCNCC5)ccc4n3)cc(C)c2n1]

To a solution of tert-butyl 4-[2-(2,8-dimethylimidazo[1,2-b]pyridazin-6-yl)-5-oxo-1,6-naphthyridin-6-yl]piperidine-1-carboxylate (80 mg, 168.58 umol, 1 eq) in ethyl acetate (0.1 mL) was added HCl/ethyl acetate (4 M, 830.37 uL, 19.70 eq). The reaction mixture was stirred at 25° C. for 1 h, then filtered and the filter cake was diluted with water (10 mL). The aqueous solution was lyophilized to give 2-(2,8-dimethylimidazo[1,2-b]pyridazin-6-yl)-6-(4-piperidyl)-1,6-naphthyridin-5-one (60 mg, 87%) as a solid. LCMS: (ES, m/z): 375.2 [M+H]^{+ 1}HNMR (400 MHz, D₂O) δ ppm 8.71 (br d, J=8.44 Hz, 1H), 8.35 (br s, 1H), 8.28 (br d, J=7.95 Hz, 1H), 8.10 (br s, 1H), 7.75 (br d, J=7.46 Hz, 1H), 7.01 (br d, J=7.58 Hz, 1H), 5.10 (br s, 1H), 3.65 (br d, J=12.84 Hz, 2H), 3.28 (br t, J=11.31 Hz, 2H), 2.69 (br s, 3H), 2.54 (br s, 3H), 2.21-2.29 (m, 4H).

### Example G124: Synthesis of Compound 1500

### Synthesis of Intermediate G252

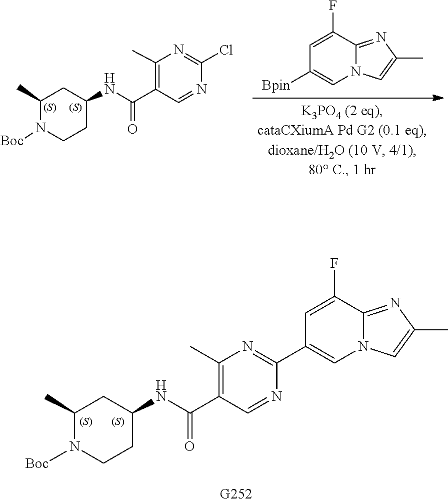

A mixture of tert-butyl (2R,4R)-4-[(2-chloro-4-methyl-pyrimidine-5-carbonyl)amino]-2-methyl-piperidine-1-carboxylate (200 mg, 542 umol, 1 eq), 8-fluoro-2-methyl-6-(4,4,5,5-tetramethyl-1,3,2-dioxaborolan-2-yl)imidazo[1,2-a]pyridine (224 mg, 813 umol, 1.5 eq), K₃PO₄ (360 mg, 1.69 mmol, 3 eq), and [2-(2-aminophenyl)phenyl]-chloro-palladium; bis(1-adamantyl)-butyl-phosphane (38.2 mg, 36.5 umol, 0.1 eq) in dioxane (1.60 mL) and water (400 uL) was degassed and purged with N₂ 3 times. The reaction mixture was stirred at 80° C. for 1 h under N₂ atmosphere, then cooled to 20° C., poured into water (10.0 mL), and extracted with dichloromethane (3×20.0 mL). The organic layers were combined, dried over Na₂SO₄, filtered, and the filtrate was concentrated under reduced pressure to give a residue, which was purified by column chromatography on silica gel (SiO₂, % of ethyl acetate in petroleum ether) to give tert-butyl (2S,4S)-4-[[2-(8-fluoro-2-methyl-imidazo[1,2-a]pyridin-6-yl)-4-methyl-pyrimidine-5-carbonyl]amino]-2-methyl-piperidine-1-carboxylate (180 mg, 69%) as a solid. LCMS: (ES, m/z): 483.4[M+H]⁺. ¹H NMR (400 MHz, METHANOL-d₄) δ ppm 9.36 (d, J=1.25 Hz, 1H) 8.74 (s, 1H) 8.02 (dd, J=12.05, 1.25 Hz, 1H) 7.85 (d, J=2.13 Hz, 1H) 4.16 (s, 2H) 3.75-3.87 (m, 1H) 3.34-3.38 (m, 1H) 2.69 (s, 3H) 2.47 (s, 3H) 1.98-2.16 (m, 2H) 1.82-1.89 (m, 1H) 1.65-1.75 (m, 1H) 1.50 (s, 9H) 1.32 (d, J=6.78 Hz, 3H).

### Synthesis of Intermediate G253

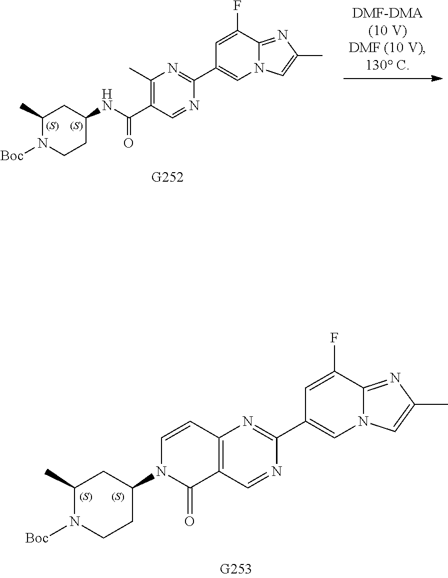

To a solution of tert-butyl (2S,4S)-4-[[2-(8-fluoro-2-methyl-imidazo[1,2-a]pyridin-6-yl)-4-methyl-pyrimidine-5-carbonyl]amino]-2-methyl-piperidine-1-carboxylate (180 mg, 373 umol, 1 eq) in N,N-dimethylformamide (1.80 mL) was added N,N-dimethylformamide-dimethylacetamide (1.80 mL) under N₂ atmosphere. The resulting mixture was stirred at 130° C. for 2 h, then cooled to 25° C., and concentrated under reduced pressure to give a residue, which was purified by column chromatography on silica gel (SiO₂, 59% of ethyl acetate in petroleum ether) to give tert-butyl (2S,4S)-4-[2-(8-fluoro-2-methyl-imidazo[1,2-a]pyridin-6-yl)-5-oxo-pyrido[4,3-d]pyrimidin-6-yl]-2-methyl-piperidine-1-carboxylate (130 mg, 71%) as a solid. LCMS: (ES, m/z): 493.3 [M+H]⁺. ¹HNMR (400 MHz, METHANOL-d₄) δ ppm 9.59 (s, 1H) 9.46 (s, 1H) 8.09 (d, J=11.76 Hz, 1H) 7.99 (d, J=7.63 Hz, 1H) 7.86 (d, J=2.38 Hz, 1H) 6.83 (d, J=7.75 Hz, 1H) 4.94-5.00 (m, 1H) 3.97-4.10 (m, 1H) 3.79-3.90 (m, 1H) 3.45-3.51 (m, 1H) 2.46 (s, 3H) 2.26-2.38 (m, 1H) 2.07-2.22 (m, 1H) 1.93-2.03 (m, 1H) 1.80 (dt, J=6.41, 3.24 Hz, 1H) 1.51 (s, 9H) 1.31 (d, J=6.38 Hz, 3H).

### Synthesis of Compound 1500

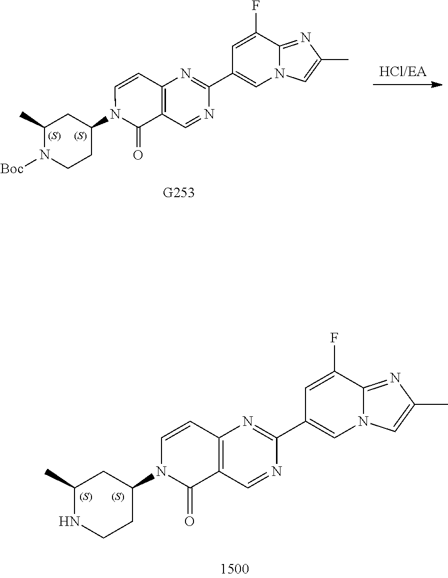

To a mixture of tert-butyl (2S,4S)-4-[2-(8-fluoro-2-methyl-imidazo[1,2-a]pyridin-6-yl)-5-oxo-pyrido[4,3-d]pyrimidin-6-yl]-2-methyl-piperidine-1-carboxylate (130 mg, 263 umol, 1 eq) in ethyl acetate (1.30 mL) was added HCl/ethyl acetate (4.00 M, 1.30 mL). The reaction mixture was stirred at 25° C. for 2 h, then filtered and the filter cake was concentrated under reduced pressure to give a residue. The residue was triturated with dichloromethane (5 mL) to give a suspension. The suspension was filtered and the filter cake was dried to give 2-(8-fluoro-2-methyl-imidazo[1,2-a]pyridin-6-yl)-6-[(2S,4S)-2-methyl-4-piperidyl]pyrido[4,3-d]pyrimidin-5-one (77.9 mg, 75%) as a solid. LCMS: (ES, m/z): 393.2 [M+H]⁺. ¹H NMR (400 MHz, METHANOL-d₄) δ ppm 9.77 (d, J=0.88 Hz, 1H) 9.68 (s, 1H) 8.74 (dd, J=11.17, 0.88 Hz, 1H) 8.22 (d, J=1.00 Hz, 1H) 7.97 (d, J=7.78 Hz, 1H) 6.90 (d, J=7.65 Hz, 1H) 5.05-5.16 (m, 1H) 3.59-3.69 (m, 1H) 3.47-3.57 (m, 1H) 3.27 (br s, 1H) 2.62 (d, J=0.75 Hz, 3H) 2.11-2.37 (m, 4H) 1.45 (d, J=6.40 Hz, 3H) ¹⁹F NMR (376 MHz, METHANOL-d₄) δ ppm−134.034 (s, 1F).

### Example G125: Synthesis of Compound 1502

### Synthesis of Intermediate G254

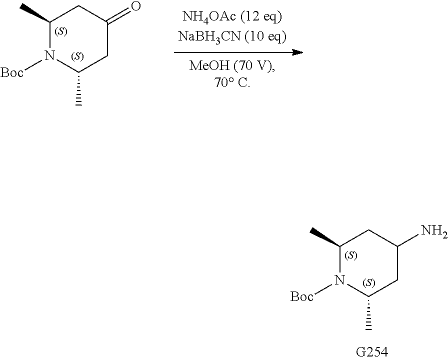

To a solution of tert-butyl (2S,6S)-2,6-dimethyl-4-oxo-piperidine-1-carboxylate (380 mg, 1.67 mmol, 1 eq) in methanol (25.0 mL) was added dropwise NH₄OAc (1.55 g, 20.0 mmol, 12 eq) at 25° C. The reaction mixture was stirred for 10 min, then NaBH₃CN (1.05 g, 16.7 mmol, 10.0 eq) was added dropwise at 0° C. The resulting mixture was stirred at 70° C. for 1 h, then quenched with water (10.0 mL) at 0° C., diluted with dichloromethane (10.0 mL), and the layers separated. The aqueous layer was extracted with dichloromethane (3×10.0 mL). The organic layers were combined, dried over Na₂SO₄, filtered, and the filtrate concentrated under reduced pressure to give tert-butyl (2S,6S)-4-amino-2,6-dimethyl-piperidine-1-carboxylate (400 mg, 16%) as a solid. LCMS: (ES, m/z): 229.4 [M+H]⁺.

### Synthesis of Intermediate G255

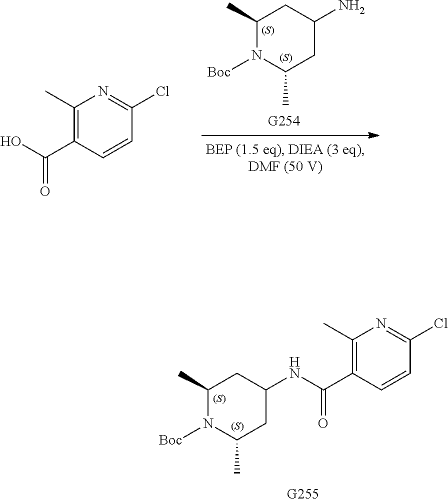

To a solution of 6-chloro-2-methyl-pyridine-3-carboxylic acid (200 mg, 1.17 mmol, 1 eq) in DMF (10.0 mL) was added 2-bromo-1-ethyl-pyridin-1-ium; tetrafluoroborate (383 mg, 1.40 mmol, 1.2 eq), tert-butyl (2S,6S)-4-amino-2,6-dimethylpiperidine-1-carboxylate (0144-1, 266 mg, 1.17 mmol, 1 eq) and N-ethyl-N-propan-2-ylpropan-2-amine (376 mg, 2.91 mmol, 507 uL, 2.5 eq). The reaction mixture was stirred at 30° C. for 2 h, then quenched with water (10.0 mL) at 0° C., diluted with ethyl acetate (10.0 mL), and the layers separated. The aqueous layer was extracted with ethyl acetate (3×10.0 mL). The organic layers were combined, washed with brine (3×10.0 mL), dried over Na₂SO₄, filtered, and the filtrate concentrated under reduced pressure to give a residue. The residue was purified by column chromatography (SiO₂, Petroleum ether/Ethyl acetate=5/1 to 0/1) to give tert-butyl (2S,6S)-4-[(6-chloro-2-methyl-pyridine-3-carbonyl)amino]-2,6-dimethyl-piperidine-1-carboxylate (200 mg, 22%) as a solid. LCMS: (ES, m/z): 382.4 [M+H]⁺. ¹H NMR (400 MHz, CHLOROFORM-d) δ ppm 8.24 (d, J=8.25 Hz, 1H), 7.62 (d, J=8.00 Hz, 1H), 6.64 (d, J=9.13 Hz, 1H), 4.42-4.55 (m, 1H), 4.29-4.40 (m, 1H), 3.78-3.91 (m, 1H), 2.65 (s, 3H), 2.36 (ddd, J=14.32, 6.82, 4.50 Hz, 1H), 2.10 (ddd, J=13.38, 6.50, 2.50 Hz, 1H), 1.70-1.83 (m, 1H), 1.54 (dt, J=14.32, 5.97 Hz, 1H), 1.47 (s, 9H), 1.39 (d, J=2.88 Hz, 3H), 1.25-1.30 (m, 3H).

### Synthesis of Intermediate G256

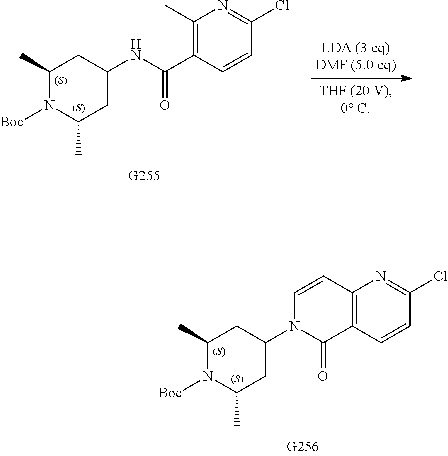

To a solution of tert-butyl (2S,6S)-4-[(6-chloro-2-methyl-pyridine-3-carbonyl)amino]-2,6-dimethyl-piperidine-1-carboxylate (200 mg, 523 umol, 1 eq) in THE (400 uL) was added LDA (2 M, 785 uL, 3 eq) dropwise at 0° C. over 3 min. The reaction mixture was stirred at 0° C. for 1 h, then DMF (950 mg, 1.00 mL) was added dropwise at 0° C. The resulting mixture was stirred at 0° C. for 20 min, then quenched with water (10.0 mL) at 0° C., diluted with ethyl acetate (10.0 mL), and the layers separated. The aqueous layer was extracted with ethyl acetate (3×10.0 mL). The organic layers were combined, washed with brine (3×10.0 mL), dried over Na₂SO₄, filtered, and the filtrate concentrated under reduced pressure to give a residue. The residue was purified by prep-TLC (SiO₂, Petroleum ether:Ethyl acetate=1:1) to give tert-butyl (2S,6S)-4-(2-chloro-5-oxo-1,6-naphthyridin-6-yl)-2,6-dimethyl-piperidine-1-carboxylate (100 mg, 44%) as a solid. LCMS: (ES, m/z): 392.2 [M+H]⁺. ¹H NMR (400 MHz, CHLOROFORM-d) δ ppm 8.61 (d, J=8.28 Hz, 1H), 7.40 (dd, J=8.09, 1.82 Hz, 2H), 6.76 (d, J=7.78 Hz, 1H), 5.33-5.58 (m, 1H), 4.55-4.69 (m, 1H), 3.73 (ddd, J=9.85, 6.59, 3.39 Hz, 1H), 1.96 (td, J=12.67, 5.40 Hz, 2H), 1.80-1.87 (m, 2H), 1.52 (d, J=6.65 Hz, 3H), 1.50 (s, 9H), 1.37 (d, J=7.03 Hz, 3H).

### Synthesis of Intermediate G257

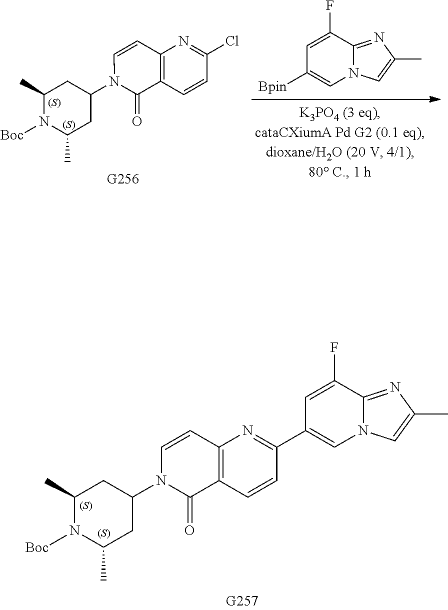

A mixture of tert-butyl (2S,6S)-4-(2-chloro-5-oxo-1,6-naphthyridin-6-yl)-2,6-dimethyl-piperidine-1-carboxylate (0139-2, 90.0 mg, 229 umol, 1 eq), 8-fluoro-2-methyl-6-(4,4,5,5-tetramethyl-1,3,2-dioxaborolan-2-yl)imidazo[1,2-a]pyridine (152 mg, 275 umol, 1.2 eq), [2-(2-aminophenyl)phenyl]-chloro-palladium; bis(1-adamantyl)-butyl-phosphane (15.3 mg, 22.9 umol, 0.1 eq), and K₃PO₄ (146 mg, 688 umol, 3 eq) in dioxane (2.00 mL) and water (500 uL) was degassed and purged with N₂ 3 times, then stirred at 80° C. for 1 h under N₂ atmosphere. The reaction mixture was filtered through celite and concentrated under reduced pressure to give a residue. The residue was purified by prep-TLC (SiO₂, Petroleum ether/Ethyl acetate=6/1 to 0/1) to give tert-butyl (2S,6S)-4-[2-(8-fluoro-2-methyl-imidazo[1,2-a]pyridin-6-yl)-5-oxo-1,6-naphthyridin-6-yl]-2,6-dimethyl-piperidine-1-carboxylate (90.0 mg, 78%) as a solid. LCMS: (ES, m/z): 506.3 [M+H]⁺. ¹H NMR (400 MHz, CHLOROFORM-d) δ ppm 8.78 (d, J=1.38 Hz, 1H), 8.74 (d, J=8.38 Hz, 1H), 7.75 (d, J=8.50 Hz, 1H), 7.64 (dd, J=11.51, 1.38 Hz, 1H), 7.52 (d, J=2.25 Hz, 1H), 7.33-7.44 (m, 2H), 6.82-6.87 (m, 1H), 5.41-5.55 (m, 1H), 4.60-4.66 (m, 1H), 3.70-3.80 (m, 1H), 2.51 (s, 3H), 1.95-2.03 (m, 2H), 1.82-1.88 (m, 2H), 1.51 (s, 9H), 1.35 (s, 3H), 1.24 (s, 3H).

### Synthesis of Compound 1502

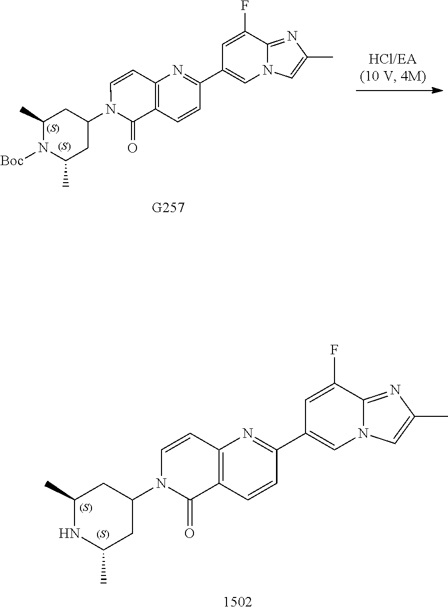

To a solution of tert-butyl (2S,6S)-4-[2-(8-fluoro-2-methyl-imidazo[1,2-a]pyridin-6-yl)-5-oxo-1,6-naphthyridin-6-yl]-2,6-dimethyl-piperidine-1-carboxylate (100 mg, 197 umol, 1 eq) in ethyl acetate (500 uL) was added HCl/EtOAc (4 M, 500 uL). The reaction mixture was stirred at 25° C. for 3 h, then concentrated under reduced pressure to give a residue. The residue was triturated with ethyl acetate (500 uL) at 25° C. for 30 min to give a suspension. The suspension was filtered and the filter cake dried to give 6-[(2S,6S)-2,6-dimethyl-4-piperidyl]-2-(8-fluoro-2-methyl-imidazo[1,2-a]pyridin-6-yl)-1,6-naphthyridin-5-one (46.7 mg, 53%) as a solid. LCMS: (ES, m/z): 406.2 [M+H]⁺. ¹H NMR (400 MHz, METHANOL-d₄) δ ppm 9.54 (d, J=1.00 Hz, 1H), 8.82 (d, J=8.53 Hz, 1H), 8.67 (dd, J=11.17, 1.13 Hz, 1H), 8.16-8.22 (m, 2H), 7.74 (d, J=7.91 Hz, 1H), 6.99 (d, J=7.78 Hz, 1H), 5.37 (tt, J=12.36, 3.45 Hz, 1H), 3.97-4.13 (m, 1H), 3.73-3.87 (m, 1H), 2.63 (s, 3H), 2.41 (td, J=13.30, 5.02 Hz, 1H), 1.99-2.11 (m, 1H), 2.12-2.27 (m, 1H), 1.60 (d, J=7.15 Hz, 3H), 1.44 (d, J=6.40 Hz, 3H). ¹⁹F NMR (376 MHz, METHANOL-d₄) δ ppm−133.954.

### Example G126: Synthesis of Compound 1503

### Synthesis of Intermediate G258

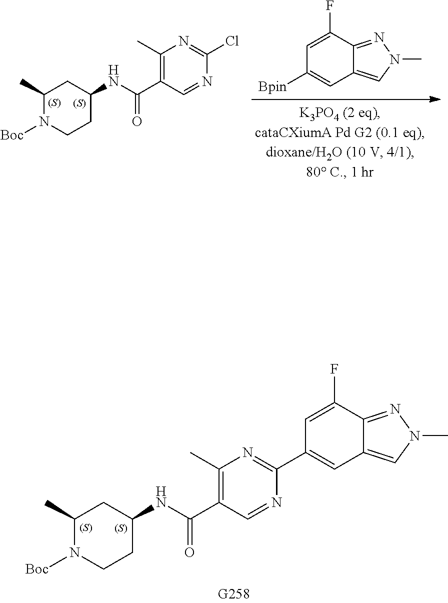

A mixture of tert-butyl (2S,4S)-4-[(2-chloro-4-methyl-pyrimidine-5-carbonyl)amino]-2-methyl-piperidine-1-carboxylate (150 mg, 407 umol, 1 eq), 7-fluoro-2-methyl-5-(4,4,5,5-tetramethyl-1,3,2-dioxaborolan-2-yl)indazole (112 mg, 406.66 umol, 1 eq), K₃PO₄ (259 mg, 1.22 mmol, 3 eq), and [2-(2-aminophenyl)phenyl]-chloro-palladium; bis(1-adamantyl)-butyl-phosphane (27.19 mg, 40.67 umol, 0.1 eq) in dioxane (2.40 mL) and water (600 uL) was degassed and purged with N₂ 3 times. The reaction mixture was stirred at 80° C. for 1 h under N₂ atmosphere, then cooled to 20° C., poured into water (10.0 mL), and extracted with dichloromethane (3×20.0 mL). The organic layers were combined, dried over Na₂SO₄, filtered, and the filtrate was concentrated under reduced pressure to give a residue, which was purified by column chromatography on silica gel (SiO₂, 65% of ethyl acetate in petroleum ether) to give tert-butyl (2S,4S)-4-[[2-(7-fluoro-2-methyl-indazol-5-yl)-4-methyl-pyrimidine-5-carbonyl]amino]-2-methyl-piperidine-1-carboxylate (90.0 mg, 46%) as a solid. LCMS: (ES, m/z): 483.4[M+H]⁺. ¹H NMR (400 MHz, METHANOL-d₄) δ ppm 8.74 (d, J=1.13 Hz, 1H) 8.71 (s, 1H) 8.47 (d, J=2.64 Hz, 1H) 8.07 (dd, J=13.18, 1.13 Hz, 1H) 4.27 (s, 3H) 4.05-4.20 (m, 3H) 3.75-3.83 (m, 1H) 2.68 (s, 3H) 2.05-2.14 (m, 1H) 1.99 (br dd, J=6.21, 4.33 Hz, 1H) 1.86 (dt, J=13.80, 7.15 Hz, 1H) 1.63-1.73 (m, 1H) 1.48 (s, 9H) 1.31 (d, J=6.78 Hz, 3H).

### Synthesis of Intermediate G259

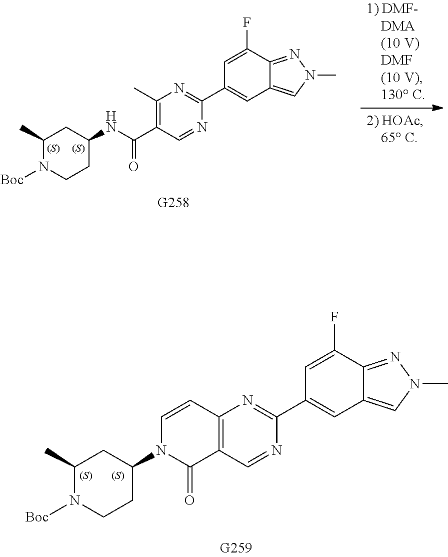

To a solution of tert-butyl (2S,4S)-4-[[2-(7-fluoro-2-methyl-indazol-5-yl)-4-methyl-pyrimidine-5-carbonyl]amino]-2-methyl-piperidine-1-carboxylate (90.0 mg, 186 umol, 1 eq) in N,N-dimethylformamide (0.900 mL) was added N,N-dimethylformamide-dimethylacetamide (0.900 mL). The resulting mixture was stirred at 130° C. for 2 h. The reaction mixture was concentrated under reduced pressure to give a residue. To the residue was added acetic acid (900 uL) and the reaction mixture was degassed and purged with N₂ 3 times, then stirred at 65° C. for 2 h under N₂ atmosphere. The reaction mixture was concentrated under reduced pressure to give a residue, which was purified by column chromatography on silica gel (SiO₂, 59% of ethyl acetate in petroleum ether) to give tert-butyl (2S,4S)-4-[2-(7-fluoro-2-methyl-indazol-5-yl)-5-oxo-pyrido[4,3-d]pyrimidin-6-yl]-2-methyl-piperidine-1-carboxylate (60.0 mg, 61%) as a solid. LCMS: (ES, m/z): 493.3 [M+H]⁺. ¹H NMR (400 MHz, METHANOL-d₄) δ ppm 9.55 (s, 1H) 8.84 (d, J=1.13 Hz, 1H) 8.49 (d, J=2.64 Hz, 1H) 8.13 (dd, J=13.18, 1.13 Hz, 1H) 7.95 (d, J=7.78 Hz, 1H) 6.82 (d, J=7.65 Hz, 1H) 4.92-5.02 (m, 1H) 4.27 (s, 3H) 4.02-4.09 (m, 1H) 3.85 (ddd, J=14.15, 7.37, 2.45 Hz, 1H) 3.44-3.52 (m, 1H) 2.26-2.43 (m, 1H) 2.08-2.22 (m, 1H) 1.92-2.01 (m, 1H) 1.75-1.85 (m, 1H) 1.51 (s, 9H) 1.29-1.32 (m, 3H).

### Synthesis of Compound 1503

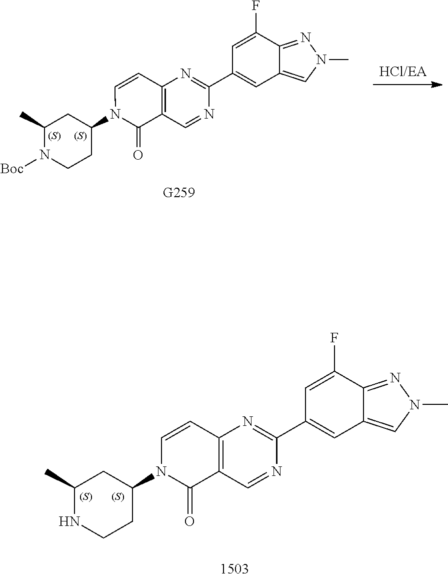

A mixture of tert-butyl (2S,4S)-4-[2-(7-fluoro-2-methyl-indazol-5-yl)-5-oxo-pyrido[4,3-d]pyrimidin-6-yl]-2-methyl-piperidine-1-carboxylate (50.0 mg, 101 umol, 1 eq) in ethyl acetate (0.500 mL) was added HCl/ethyl acetate (4.00 M, 0.500 mL). The reaction mixture was stirred at 25° C. for 2 h, then filtered and the filter cake was dried to give a residue. The residue was triturated with dichloromethane (5.00 mL) to give a suspension. The suspension was filtered and the filter cake was dried to give 2-(7-fluoro-2-methyl-indazol-5-yl)-6-[(2S,4S)-2-methyl-4-piperidyl]pyrido[4,3-d]pyrimidin-5-one (46.7 mg, 98%) as a solid. LCMS: (ES, m/z): 393.2 [M+H]⁺. ¹H NMR (400 MHz, DMSO-d₆) δ ppm 9.55 (s, 1H) 9.12 (br d, J=9.38 Hz, 1H) 8.91-9.05 (m, 1H) 8.86 (s, 1H) 8.73 (d, J=2.63 Hz, 1H) 8.06 (dd, J=13.26, 0.88 Hz, 1H) 7.88 (d, J=7.88 Hz, 1H) 6.83 (d, J=7.75 Hz, 1H) 5.02-5.14 (m, 1H) 4.26 (s, 3H) 3.45 (br d, J=11.76 Hz, 2H) 3.18 (br d, J=11.13 Hz, 1H) 2.22 (br dd, J=12.82, 3.94 Hz, 1H) 1.95-2.14 (m, 3H) 1.32 (d, J=6.38 Hz, 3H) ¹⁹F NMR (376 MHz, DMSO-d₆) δ ppm−128.783 (s, 1F).

### Example G127: Synthesis of Compound 1504

### Synthesis Intermediate G260

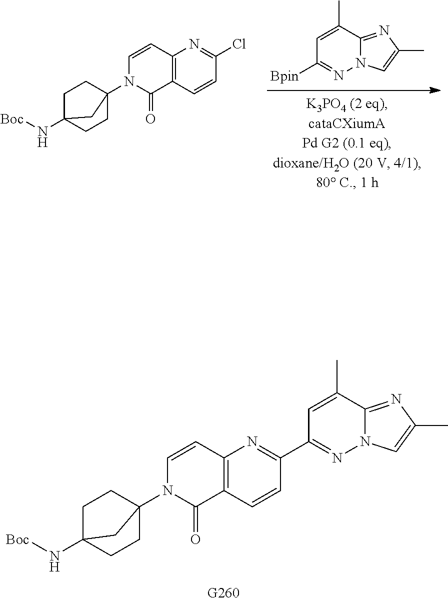

To a mixture of 2,8-dimethyl-6-(4,4,5,5-tetramethyl-1,3,2-dioxaborolan-2-yl)imidazo[1,2-b]pyridazine (208.07 mg, 761.78 umol, 2.2 eq) and tert-butyl N-[4-(2-chloro-5-oxo-1,6-naphthyridin-6-yl)norbornan-1-yl]carbamate (135.00 mg, 346.26 umol, 1 eq) in dioxane (2.4 mL) and water (0.6 mL) was added [2-(2-aminophenyl)phenyl]-chloro-palladium; bis(1-adamantyl)-butyl-phosphane (23.15 mg, 34.63 umol, 0.1 eq) and potassium phosphate (147.00 mg, 692.53 umol, 2 eq). The reaction mixture was stirred at 80° C. for 1 h under nitrogen atmosphere, then diluted with water (10 mL) and extracted with ethyl acetate (20 mL×3). The organic layers were combined, washed with brine (60 mL), dried over anhydrous sodium sulfate, filtered, and the filtrate concentrated under reduced pressure to give a residue. The residue was purified by column chromatography on silica gel, eluting with 30% ethyl acetate in petroleum ether to give tert-butyl N-[4-[2-(2,8-dimethylimidazo[1,2-b]pyridazin-6-yl)-5-oxo-1,6-naphthyridin-6-yl]norbornan-1-yl]carbamate (120 mg, 64%) as a solid. LCMS: (ES, m/z): 501.2 [M+H]⁺. ¹HNMR (400 MHz, DMSO-d6) δ ppm 8.66 (d, J=8.51 Hz, 1H), 8.34 (d, J=8.50 Hz, 1H), 8.15 (s, 1H), 8.06 (s, 1H), 7.76 (d, J=7.88 Hz, 1H), 7.15 (br s, 1H), 6.75 (d, J=7.88 Hz, 1H), 2.69 (s, 1H), 2.65 (s, 3H), 2.43 (s, 3H), 2.37 (s, 1H), 1.99-2.11 (m, 2H), 1.84-1.97 (m, 2H), 1.69-1.82 (m, 4H), 1.41 (s, 9H).

### Synthesis of Compound 1504

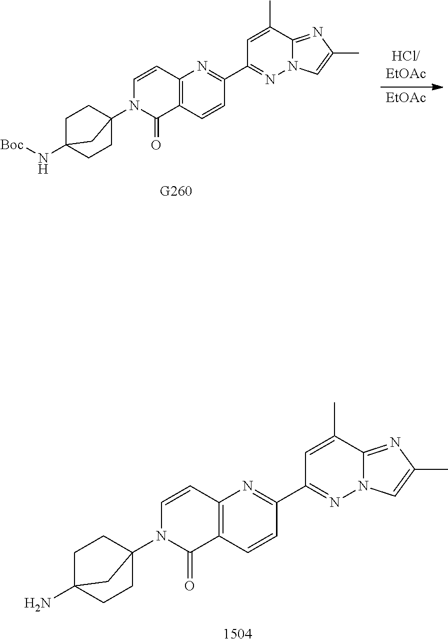 [SMILES: "EtOAc" ; Cc1cn2nc(-c3ccc4c(=O)n(C56CCC(NC(=O)OC(C)(C)C)(CC5)C6)ccc4n3)cc(C)c2n1 ; Cc1cn2nc(-c3ccc4c(=O)n(C56CCC(N)(CC5)C6)ccc4n3)cc(C)c2n1]

To a solution of tert-butyl N-[4-[2-(2,8-dimethylimidazo[1,2-b]pyridazin-6-yl)-5-oxo-1,6-naphthyridin-6-yl]norbornan-1-yl]carbamate (120 mg, 239.72 umol, 1 eq) in ethyl acetate (1.2 mL) was added HCl/ethyl acetate (4 M, 1.20 mL, 20.02 eq). The reaction mixture was stirred at 25° C. for 1 h, then concentrated under reduced pressure to give a residue. The residue was triturated with dichloromethane (2 mL) at 20° C. for 5 min to give a suspension. The suspension was filtered and the filter cake was collected with water (2 mL). The aqueous solution was lyophilized to give 6-(4-aminonorbornane-1-yl)-2-(2,8-dimethylimidazo[1,2-b]pyridazin-6-yl)-1,6-naphthyridin-5-one (97 mg, 91%) as a solid. LCMS: (ES, m/z): 401.2 [M+H]⁺. ¹HNMR (400 MHz, D₂O) δ ppm 8.67 (br d, J=8.63 Hz, 1H), 8.35 (s, 1H), 8.26 (d, J=8.50 Hz, 1H), 8.11 (s, 1H), 7.76 (br d, J=8.00 Hz, 1H), 6.91 (br d, J=7.75 Hz, 1H), 2.70 (s, 5H), 2.56 (s, 5H), 2.01-2.14 (m, 6H).

### Example G128: Synthesis of Compounds 1514 and 1515

### Synthesis of Intermediate G261

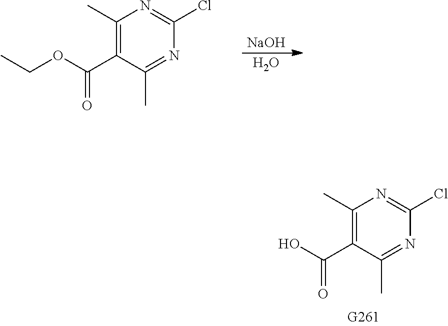

A mixture of ethyl 2-chloro-4,6-dimethyl-pyrimidine-5-carboxylate (5.00 g, 23.3 mmol, 1 eq) and NaOH (1.12 g, 27.9 mmol, 1.2 eq) in water (150 mL) was stirred at 25° C. for 3 h, then extracted with dichloromethane (3×20.0 mL). The organic layers were combined and acidified to pH 2 with aqueous HCl (6N). The resulting mixture was filtered and the filter cake was dried to give 2-chloro-4,6-dimethyl-pyrimidine-5-carboxylic acid (2.30 g, 53%) as a solid. LCMS: (ES, m/z): 187.1 [M+H]⁺. ¹H NMR (400 MHz, DMSO-d₆) δ ppm 13.95-14.26 (m, 1H) 2.49 (s, 6H).

### Synthesis of Intermediate G262

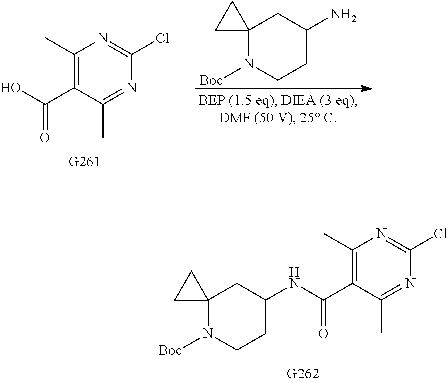 [SMILES: Cc1nc(Cl)nc(C)c1C(=O)O ; CC(C)(C)OC(=O)N1CCC(N)CC12CC2 ; Cc1nc(Cl)nc(C)c1C(=O)NC1CCN(C(=O)OC(C)(C)C)C2(CC2)C1]

A mixture of 2-chloro-4,6-dimethyl-pyrimidine-5-carboxylic acid (1.00 g, 5.35 mmol, 1 eq), tert-butyl 7-amino-4-azaspiro[2.5]octane-4-carboxylate (1.33 g, 5.90 mmol, 1.1 eq), N,N-diisopropylethylamine (2.08 g, 16.1 mmol, 3 eq), and 2-bromo-1-ethyl-pyridin-1-ium; tetrafluoroborate (2.20 g, 8.05 mmol, 1.5 eq) in N,N-dimethylformamide (50.0 mL) was degassed and purged with N₂ 3 times. The reaction mixture was stirred at 30° C. for 2 h, then quenched with water (200 mL) and extracted with ethyl acetate (4×50.0 mL). The organic layers were combined, dried over Na₂SO₄, filtered, and the filtrate was concentrated under reduced pressure to give a residue, which was purified by column chromatography (SiO2, 53% of ethyl acetate in petroleum ether) to give tert-butyl 7-[(2-chloro-4,6-dimethyl-pyrimidine-5-carbonyl)amino]-4-azaspiro[2.5]octane-4-carboxylate (900 mg, 35%) as a solid. LCMS: (ES, m/z): 339.2 [M−55+H]⁺. ¹H NMR (400 MHz, METHANOL-d₄) δ ppm 4.30 (s, 1H) 3.98-4.08 (m, 1H) 3.01-3.12 (m, 1H) 2.45 (s, 6H) 1.96-2.05 (m, 1H) 1.83-1.95 (m, 1H) 1.46 (s, 9H) 1.34-1.43 (m, 2H) 1.23 (br d, J=6.75 Hz, 1H) 0.90 (s, 1H) 0.57-0.73 (m, 2H).

### Synthesis of Intermediate G263

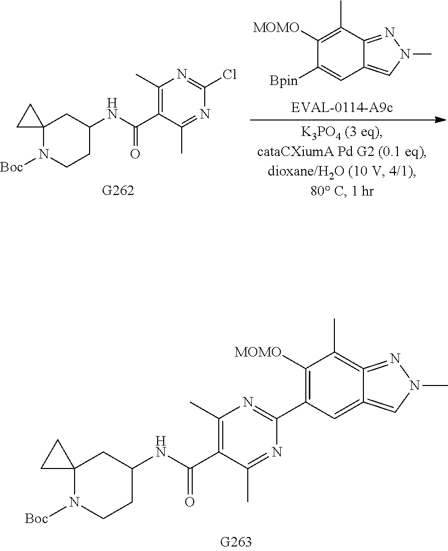

A mixture of tert-butyl 7-[(2-chloro-4,6-dimethyl-pyrimidine-5-carbonyl)amino]-4-azaspiro[2.5]octane-4-carboxylate (900 mg, 2.28 mmol, 1 eq), 6-(methoxymethoxy)-2,7-dimethyl-5-(4,4,5,5-tetramethyl-1,3,2-dioxaborolan-2-yl) indazole (907 mg, 2.73 mmol, 1.2 eq), K₃PO₄ (1.45 g, 6.84 mmol, 3 eq), and [2-(2-aminophenyl)phenyl]-chloro-palladium; bis(1-adamantyl)-butyl-phosphane (152 mg, 228 umol, 0.1 eq) in dioxane (7.20 mL) and water (1.80 mL) was degassed and purged with N₂ 3 times, then stirred at 80° C. for 1 h under N₂ atmosphere. The resulting mixture was cooled to 20° C., then poured into water (20.0 mL) and extracted with dichloromethane (3×10.0 mL). The organic layers were combined, dried over Na₂SO₄, filtered, and the filtrate was concentrated under reduced pressure to give a residue, which was purified by column chromatography on silica gel (SiO₂, 82% of ethyl acetate in petroleum ether) to give tert-butyl 7-[[2-[6-(methoxymethoxy)-2,7-dimethyl-indazol-5-yl]-4,6-dimethyl-pyrimidine-5-carbonyl]amino]-4-azaspiro[2.5]octane-4-carboxylate (800 mg, 63%) as a solid. LCMS: (ES, m/z): 565.5[M+H]⁺. ¹H NMR (400 MHz, METHANOL-d₄) δ ppm 8.27 (s, 1H) 7.84 (s, 1H) 4.83 (s, 2H) 4.31-4.41 (m, 1H) 4.21-4.25 (m, 3H) 4.01-4.08 (m, 1H) 3.30 (br s, 3H) 3.02-3.12 (m, 1H) 2.58 (s, 3H) 2.53 (s, 6H) 2.05 (br s, 1H) 1.94 (s, 1H) 1.50 (br d, J=4.25 Hz, 1H) 1.47 (s, 9H) 1.42 (dd, J=13.20, 4.06 Hz, 1H) 1.25-1.32 (m, 1H) 0.86-0.99 (m, 1H) 0.57-0.74 (m, 2H).

### Synthesis of Intermediate G264

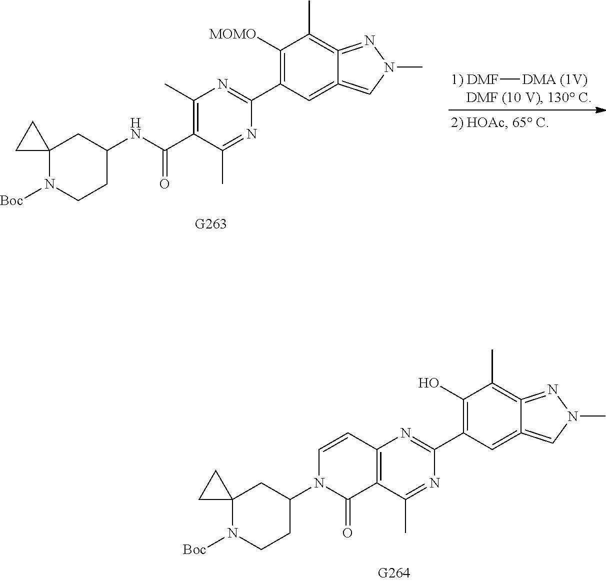

To a solution of tert-butyl 7-[[2-[6-(methoxymethoxy)-2,7-dimethyl-indazol-5-yl]-4,6-dimethyl-pyrimidine-5-carbonyl]amino]-4-azaspiro[2.5]octane-4-carboxylate (800 mg, 1.42 mmol, 1 eq) in N,N-dimethylformamide (8.00 mL) was added 1,1-dimethoxy-N,N-dimethylmethanamine (800 uL). The resulting mixture was stirred at 130° C. for 2 h, then cooled to 25° C. and concentrated under reduced pressure to give a residue. To the residue was added acetic acid (8.00 mL). The reaction mixture was degassed and purged with N₂ 3 times and stirred at 65° C. for 2 h under N₂ atmosphere, then concentrated under reduced pressure to give a residue, which was purified by column chromatography on silica gel (SiO₂, 84% of ethyl acetate in petroleum ether) to give tert-butyl 7-[2-(6-hydroxy-2,7-dimethyl-indazol-5-yl)-4-methyl-5-oxo-pyrido[4,3-d]pyrimidin-6-yl]-4-azaspiro[2.5]octane-4-carboxylate (350 mg, 43%) as a solid. LCMS: (ES, m/z): 531.4 [M+H]⁺. ¹HNMR (400 MHz, METHANOL-d₄) δ ppm 8.87 (s, 1H) 8.20 (s, 1H) 7.81 (d, J=7.75 Hz, 1H) 6.59 (d, J=7.63 Hz, 1H) 5.17 (s, 1H) 4.18-4.24 (m, 1H) 4.16 (s, 3H) 3.07-3.16 (m, 1H) 2.96-3.01 (m, 3H) 2.38 (s, 3H) 2.25-2.34 (m, 1H) 1.82-1.95 (m, 2H) 1.52 (s, 9H) 1.32-1.39 (m, 1H) 1.29 (br d, J=2.88 Hz, 1H) 0.98 (br dd, J=7.00, 2.88 Hz, 1H) 0.64-0.75 (m, 2H).

### Synthesis of Intermediate G265 and G266

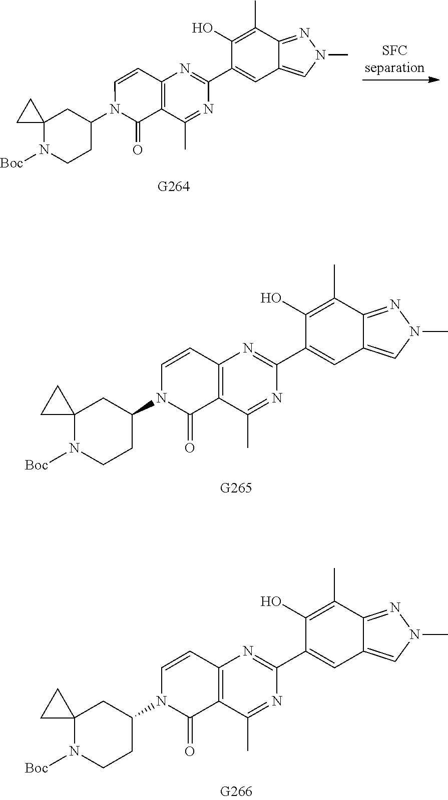 [SMILES: Cc1c(O)c(-c2nc(C)c3c(=O)n(C4CCN(C(=O)OC(C)(C)C)C5(CC5)C4)ccc3n2)cc2cn(C)nc12 ; Cc1c(O)c(-c2nc(C)c3c(=O)n([C@H]4CCN(C(=O)OC(C)(C)C)C5(CC5)C4)ccc3n2)cc2cn(C)nc12 ; Cc1c(O)c(-c2nc(C)c3c(=O)n([C@@H]4CCN(C(=O)OC(C)(C)C)C5(CC5)C4)ccc3n2)cc2cn(C)nc12]

Tert-butyl 7-[2-(6-hydroxy-2,7-dimethyl-indazol-5-yl)-4-methyl-5-oxo-pyrido[4,3-d]pyrimidin-6-yl]-4-azaspiro[2.5]octane-4-carboxylate (350 mg) was purified by SFC (Condition G5) to tert-butyl (7S)-7-[2-(6-hydroxy-2,7-dimethyl-indazol-5-yl)-4-methyl-5-oxo-pyrido[4,3-d]pyrimidin-6-yl]-4-azaspiro[2.5]octane-4-carboxylate (150 mg, 43%) and tert-butyl (7R)-7-[2-(6-hydroxy-2,7-dimethyl-indazol-5-yl)-4-methyl-5-oxo-pyrido[4,3-d]pyrimidin-6-yl]-4-azaspiro[2.5]octane-4-carboxylate (150 mg, 43%) as a solid. G265: SFC (Rt=1.580 min). LCMS (ESI, m/z) m/z: [M+H]⁺ 531.4. ¹H NMR (400 MHz, METHANOL-d₄) δ ppm 8.98 (s, 1H) 8.25 (s, 1H) 7.87 (d, J=7.75 Hz, 1H) 6.68 (d, J=7.75 Hz, 1H) 5.22 (br t, J=4.19 Hz, 1H) 4.19-4.25 (m, 1H) 4.18 (s, 3H) 3.09-3.18 (m, 1H) 3.07 (s, 3H) 2.42 (s, 3H) 2.32 (brt, J=12.76 Hz, 1H) 1.85-1.98 (m, 2H) 1.52 (s, 9H) 1.26-1.38 (m, 2H) 0.93-1.03 (m, 1H) 0.63-0.79 (m, 2H) G266: SFC (Rt=2.182 min). LCMS (ESI, m/z) m/z: [M+H]⁺ 531.4. ¹H NMR (400 MHz, METHANOL-d₄) δ ppm 8.98 (s, 1H) 8.25 (s, 1H) 7.87 (d, J=7.75 Hz, 1H) 6.68 (d, J=7.63 Hz, 1H) 5.22 (br t, J=4.44 Hz, 1H) 4.19-4.25 (m, 1H) 4.18 (s, 3H) 3.09-3.17 (m, 1H) 3.07 (s, 3H) 2.42 (s, 3H) 2.32 (br t, J=12.13 Hz, 1H) 1.83-1.95 (m, 2H) 1.52 (s, 9H) 1.29-1.40 (m, 2H) 0.92-1.02 (m, 1H) 0.62-0.76 (m, 2H).

### Synthesis of Compound 1514

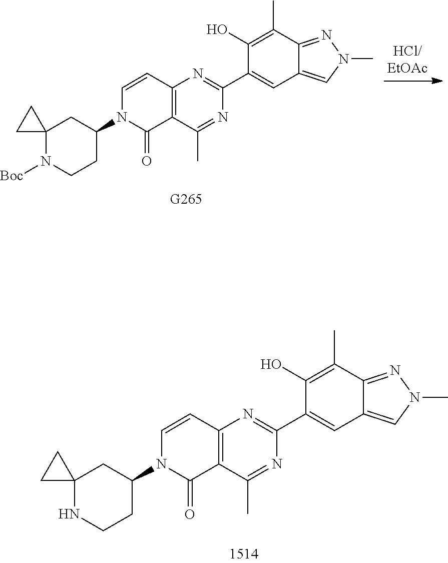 [SMILES: Cc1c(O)c(-c2nc(C)c3c(=O)n([C@H]4CCN(C(=O)OC(C)(C)C)C5(CC5)C4)ccc3n2)cc2cn(C)nc12 ; Cc1c(O)c(-c2nc(C)c3c(=O)n([C@H]4CCNC5(CC5)C4)ccc3n2)cc2cn(C)nc12]

To a mixture of tert-butyl (7S)-7-[2-(6-hydroxy-2,7-dimethyl-indazol-5-yl)-4-methyl-5-oxo-pyrido[4,3-d]pyrimidin-6-yl]-4-azaspiro[2.5]octane-4-carboxylate (150 mg, 282 umol, 1 eq) in ethyl acetate (1.50 mL) was added HCl/ethyl acetate (4.00 M, 1.50 mL). The reaction mixture was stirred at 25° C. for 2 h, then concentrated under reduced pressure to give a residue. The residue was triturated with dichloromethane (5.00 mL) to give a suspension. The suspension was filtered and the filter cake was dried to give 6-[(7S)-4-azaspiro[2.5]octan-7-yl]-2-(6-hydroxy-2,7-dimethyl-indazol-5-yl)-4-methyl-pyrido[4,3-d]pyrimidin-5-one (121 mg, 96%) as a solid. LCMS (ESI+) m/z: [M+H]⁺ 431.3. ¹H NMR (400 MHz, METHANOL-d₄) δ ppm 9.10 (s, 1H) 8.56 (s, 1H) 7.91 (d, J=7.78 Hz, 1H) 6.75 (d, J=7.65 Hz, 1H) 5.10-5.23 (m, 1H) 4.27 (s, 3H) 3.57-3.70 (m, 1H) 3.41 (td, J=12.86, 3.26 Hz, 1H) 3.09 (s, 3H) 2.74 (t, J=12.99 Hz, 1H) 2.43 (s, 3H) 2.23-2.40 (m, 2H) 1.59-1.69 (m, 1H) 1.17 (s, 2H) 0.96-1.08 (m, 2H).

### Synthesis of Compound 1515

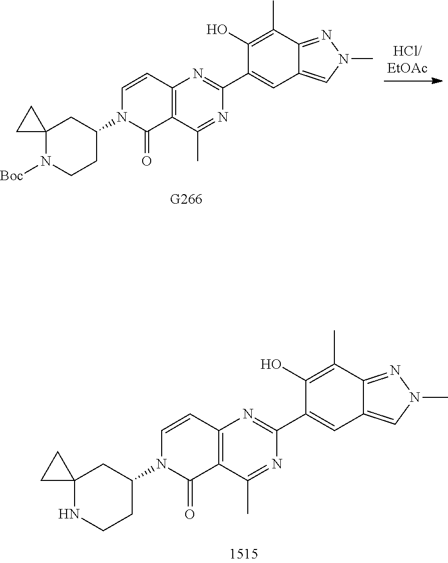 [SMILES: Cc1c(O)c(-c2nc(C)c3c(=O)n([C@@H]4CCN(C(=O)OC(C)(C)C)C5(CC5)C4)ccc3n2)cc2cn(C)nc12 ; Cc1c(O)c(-c2nc(C)c3c(=O)n([C@@H]4CCNC5(CC5)C4)ccc3n2)cc2cn(C)nc12]

To a mixture of tert-butyl (7R)-7-[2-(6-hydroxy-2,7-dimethyl-indazol-5-yl)-4-methyl-5-oxo-pyrido[4,3-d]pyrimidin-6-yl]-4-azaspiro[2.5]octane-4-carboxylate (150 mg, 282 umol, 1 eq) in ethyl acetate (1.50 mL) was added HCl/ethyl acetate (4.00 M, 1.50 mL). The reaction mixture was stirred at 25° C. for 2 h, then concentrated under reduced pressure to give a residue. The residue was triturated with dichloromethane (5.00 mL) to give a suspension. The suspension was filtered and the filter cake was dried to give (R)-2-(6-hydroxy-2,7-dimethyl-2H-indazol-5-yl)-4-methyl-6-(4-azaspiro[2.5]octan-7-yl)pyrido[4,3-d]pyrimidin-5(6H)-one (105 mg, 86%) as a solid. LCMS (ESI+) m/z: [M+H]⁺ 431.3. ¹H NMR (400 MHz, METHANOL-d₄) δ ppm 9.16 (s, 1H) 8.65 (s, 1H) 7.92 (d, J=7.78 Hz, 1H) 6.78 (d, J=7.65 Hz, 1H) 5.16 (s, 1H) 4.30 (s, 3H) 3.59-3.68 (m, 1H) 3.41 (td, J=12.89, 3.33 Hz, 1H) 3.11 (s, 3H) 2.75 (t, J=12.99 Hz, 1H) 2.45 (s, 3H) 2.23-2.40 (m, 2H) 1.64 (br dd, J=15.25, 1.69 Hz, 1H) 1.12-1.22 (m, 2H) 0.97-1.10 (m, 2H).

### Example G130: Synthesis of Compound 1524

### Synthesis of Intermediate G267

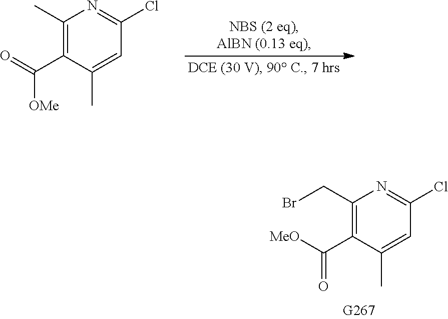

A solution of methyl 6-chloro-2,4-dimethyl-pyridine-3-carboxylate (500 mg, 2.50 mmol, 1.00 eq) in dichloromethane (15.0 mL) was stirred at 90° C. for 5 min. To the reaction mixture was added N-bromosuccinimide (891 mg, 5.01 mmol, 2.00 eq) and 2,2-Azobis(2-methylpropionitrile)(53.4 mg, 325 umol, 0.13 eq) at 90° C. under N₂. The reaction mixture was stirred at 90° C. for 7 h, then concentrated under reduced pressure to a residue. The residue was diluted with saturated NaHCO₃ (40.0 mL) and extracted with dichloromethane (3×40.0 mL). The organic layers were combined, washed with brine (100 mL), dried over anhydrous Na₂SO₄, filtered, and the filtrate concentrated to give a residue. The residue was purified by reversed-phase chromatography (0.1% NH₄HCO₃) to give methyl 2-(bromomethyl)-6-chloro-4-methyl-pyridine-3-carboxylate (145-8, 220 mg, 31.5%) as an oil. LCMS: (ESI, m/z): 278.0, 280.1 [M+H]⁺. ¹H NMR (400 MHz, METHANOL-d₄) δ ppm 7.38 (s, 1H) 4.61 (s, 2H) 3.98 (s, 3H) 2.39 (s, 3H).

### Synthesis of Intermediate G268

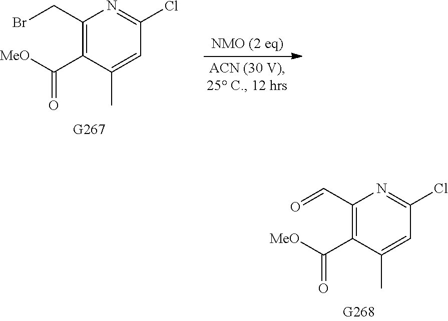

To a solution of methyl 2-(bromomethyl)-6-chloro-4-methyl-pyridine-3-carboxylate (145-8, 200 mg, 574 umol, 1.00 eq) in acetonitrile (6.00 mL) was added 4-methylmorpholine-oxide (134 mg, 1.15 mmol, 2.00 eq) at 25° C. under N₂. The reaction mixture was stirred at 25° C. for 12 h, then poured into water (20.0 mL) and extracted with ethyl acetate (3×20.0 mL). The organic layers were combined, washed with brine (50.0 mL), dried with anhydrous Na₂SO₄, filtered, and the filtrate concentrated to give a residue. The residue was purified by silica gel column chromatography (petroleum ether/ethyl acetate=4/1) to give methyl 6-chloro-2-formyl-4-methyl-pyridine-3-carboxylate (50.0 mg, 41%) as an oil. LCMS: (ESI, m/z): 214.1 [M+H]⁺. ¹H NMR (400 MHz, DMSO-d₆) δ ppm 9.85 (s, 1H) 7.90 (d, J=0.63 Hz, 1H) 3.88 (s, 3H) 2.34 (s, 3H).

### Synthesis of Intermediate G269

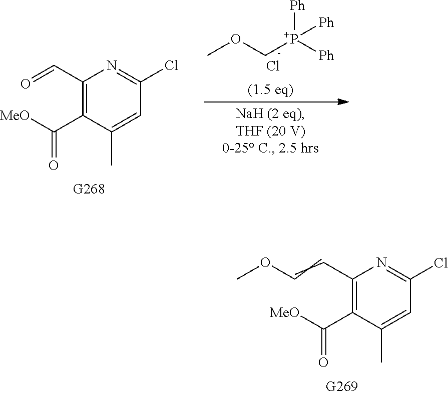 [SMILES: COC[P+](c1ccccc1)(c1ccccc1)c1ccccc1 ; COC(=O)c1c(C)cc(Cl)nc1C=O ; Cl ; COC=Cc1nc(Cl)cc(C)c1C(=O)OC]

To a solution of methoxymethyl(triphenyl)phosphonium; chloride (240 mg, 702 umol, 1.50 eq) in tetrahydrofuran (4.80 mL) was added NaH (37.4 mg, 936 umol, 2.00 eq) at 0° C. under N₂ atmosphere. The reaction mixture was stirred at 25° C. for 30 min. To the reaction mixture was added methyl 6-chloro-2-formyl-4-methyl-pyridine-3-carboxylate (145-9, 100 mg, 468 umol, 1.00 eq) in tetrahydrofuran (1.20 mL) at 25° C. under N₂ atmosphere. The resulting mixture was stirred at 25° C. for 2 h. Four additional reaction mixtures were set up in parallel and the five reaction mixtures were combined. The combined reaction mixture was poured into water (60.0 mL) and extracted with dichloromethane (3×60.0 mL). The organic layers were combined, washed with brine (60.0 mL), dried over anhydrous Na₂SO₄, filtered, and the filtrate concentrated to give a residue. The residue was purified by silica gel column chromatography (petroleum ether/ethyl acetate=7/1) to give methyl 6-chloro-2-[(E)-2-methoxyvinyl]-4-methyl-pyridine-3-carboxylate (170 mg, 30%) as a solid. LCMS: (ESI, m/z): 242.2 [M+H]⁺. ¹H NMR (400 MHz, DMSO-d₆) δ ppm 7.67 (d, J=12.01 Hz, 1H) 7.21 (s, 1H) 5.74 (d, J=12.01 Hz, 1H) 3.90 (s, 3H) 3.71 (s, 3H) 2.23 (s, 3H).

### Synthesis of Intermediate G270

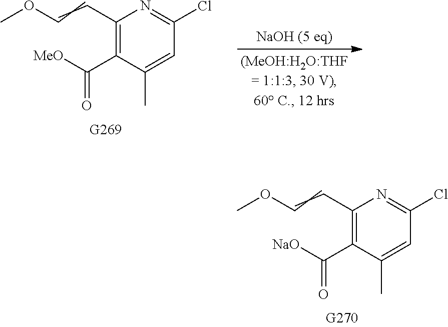

To a solution of methyl 6-chloro-2-[(E)-2-methoxyvinyl]-4-methyl-pyridine-3-carboxylate (170 mg, 703 umol, 1.00 eq) in a mixture of methanol (1.70 mL), water (1.70 mL) and tetrahydrofuran (5.10 mL) was added NaOH (140 mg, 3.52 mmol, 5.00 eq) at 25° C. under N₂ atmosphere. The reaction mixture was stirred at 60° C. for 12 h, then concentrated under reduced pressure to give [6-chloro-2-[(E)-2-methoxyvinyl]-4-methyl-pyridine-3-carbonyl]oxysodium (350 mg, 100%) as an oil. LCMS: (ESI, m/z): 228.2 [M+H]⁺. ¹H NMR (400 MHz, DMSO-d₆) δ ppm 7.47 (d, J=12.38 Hz, 1H) 6.87 (s, 1H) 6.00 (d, J=12.38 Hz, 1H) 3.62 (s, 3H) 3.19 (s, 3H).

### Synthesis of Intermediate G271

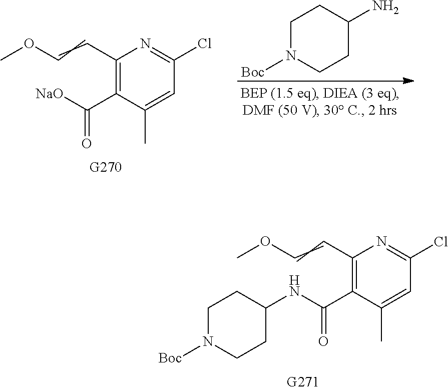

To a mixture of [6-chloro-2-[(E)-2-methoxyvinyl]-4-methyl-pyridine-3-carbonyl]oxysodium (350 mg, 1.40 mmol, 1.00 eq) and tert-butyl 4-aminopiperidine-1-carboxylate (280 mg, 1.40 mmol, 1.00 eq) in dimethyl formamide (17.5 mL) was added 2-bromo-1-ethyl-pyridin-1-ium; tetrafluoroborate (575 mg, 2.10 mmol, 1.50 eq) and N,N-diisopropylethylamine (543 mg, 4.21 mmol, 3.00 eq) at 25° C. The reaction mixture was stirred at 30° C. for 2 h, then poured into water (100 mL) and extracted with ethyl acetate (3×100 mL). The organic layers were combined, washed with brine (200 mL), dried over anhydrous Na₂SO₄, filtered, and the filtrate concentrated to give a residue. The residue was purified by silica gel column chromatography (ethyl acetate) to give tert-butyl 4-[[6-chloro-2-[(E)-2-methoxyvinyl]-4-methyl-pyridine-3-carbonyl]amino]piperidine-1-carboxylate (200 mg, 35%) as an oil. LCMS: (ESI, m/z): 410.3. ¹H NMR (DMSO-d₆, 400 MHz): δ (ppm) 8.40-8.58 (m, 1H) 7.63 (d, J=12.1 Hz, 0.5H) 7.33 (s, 0.5H) 7.14 (s, 0.5H) 5.68 (d, J=12.1 Hz, 0.5H) 3.94-4.05 (m, 1H) 3.86 (br d, J=10.4 Hz, 2H) 3.22 (s, 3H) 2.91 (br d, J=13.5 Hz, 2H) 2.16-2.31 (m, 3H) 1.81 (br dd, J=8.8, 4.3 Hz, 2H) 1.39 (s, 9H) 1.26-1.34 (m, 2H).

### Synthesis of Intermediate G272

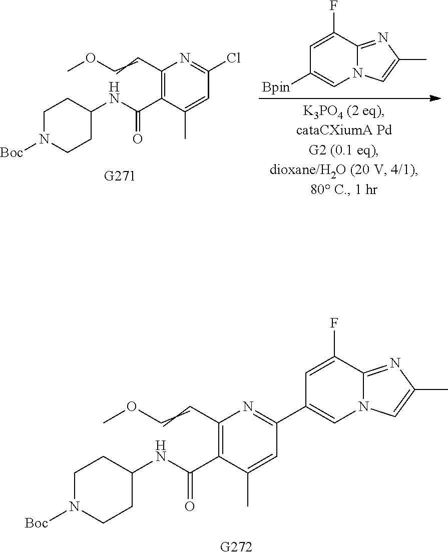

To a mixture of tert-butyl 4-[[6-chloro-2-[(E)-2-methoxyvinyl]-4-methyl-pyridine-3-carbonyl]amino]piperidine-1-carboxylate (200 mg, 487 umol, 1.00 eq) and 8-fluoro-2-methyl-6-(4,4,5,5-tetramethyl-1,3,2-dioxaborolan-2-yl)imidazo[1,2-a]pyridine (269 mg, 975 umol, 2.00 eq) in 1,4-dioxane (3.20 mL) and water (0.80 mL) was added K₃PO₄ (207 mg, 975 umol, 2.00 eq) and [2-(2-aminophenyl)phenyl]-chloro-palladium; bis(1-adamantyl)-butyl-phosphane (32.6 mg, 48.7 umol, 0.10 eq) at 25° C. under N₂ atmosphere. The reaction mixture was stirred at 80° C. for 1 h, then cooled to 25° C., poured into water (20.0 mL), and extracted with ethyl acetate (3×20.0 mL). The organic layers were combined, washed with brine (30.0 mL), dried over anhydrous Na₂SO₄, filtered, and the filtrate concentrated to give a residue. The residue was purified by silica gel column chromatography (petroleum ether/ethyl acetate=2/1) to give tert-butyl 4-[[6-(8-fluoro-2-methyl-imidazo[1,2-a]pyridin-6-yl)-2-(2-methoxyvinyl)-4-methyl-pyridine-3-carbonyl]amino]piperidine-1-carboxylate (150 mg, 59%) as a solid. LCMS: (ESI, m/z): 524.4 [M+H]⁺. ¹H NMR (DMSO-d₆, 400 MHz): δ (ppm) 9.11-9.25 (m, 1H), 8.41-8.55 (m, 1H), 7.95-8.01 (m, 1H), 7.97 (d, J=12.2 Hz, 0.5H), 7.91 (dd, J=17.4, 2.8 Hz, 1H), 7.60-7.87 (m, 2H), 5.78 (d, J=12.2 Hz, 0.5H), 3.96-4.01 (m, 1H), 3.85-3.92 (m, 2H), 3.26 (s, 3H), 2.85-3.01 (m, 3H), 2.38 (s, 2H), 2.30-2.33 (m, 2H), 2.26 (s, 3H), 1.88-1.94 (m, 2H), 1.79-1.87 (m, 2H) 1.40 (s, 9H) ¹⁹F NMR (DMSO-d₆, 376 MHz): δ (ppm)−132.279 (s, 1F).

### Synthesis of Compound 1524

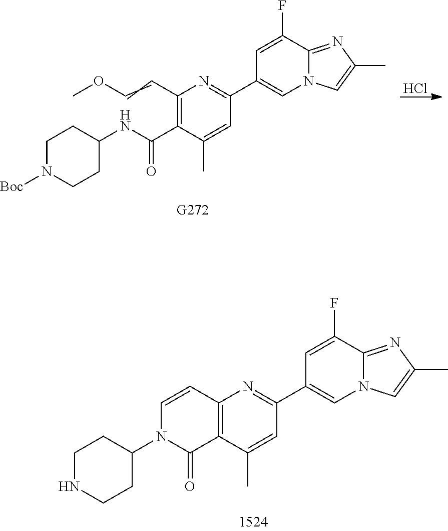 [SMILES: COC=Cc1nc(-c2cc(F)c3nc(C)cn3c2)cc(C)c1C(=O)NC1CCN(C(=O)OC(C)(C)C)CC1 ; Cl ; Cc1cn2cc(-c3cc(C)c4c(=O)n(C5CCNCC5)ccc4n3)cc(F)c2n1]

A solution of tert-butyl 4-[[6-(8-fluoro-2-methyl-imidazo[1,2-a]pyridin-6-yl)-2-(2-methoxyvinyl)-4-methyl-pyridine-3-carbonyl]amino]piperidine-1- carboxylate (80.0 mg, 152 umol, 1.00 eq) in HCl (1.60 mL, 12N) was stirred at 25° C. for 1 h. The reaction mixture was concentrated under reduced pressure to give a residue which was purified by prep-HPLC (Condition G8, Gradient 6) to give 2-(8-fluoro-2-methyl-imidazo[1,2-a]pyridin-6-yl)-4-methyl-6-(4-piperidyl)-1,6-naphthyridin-5-one (42.1 mg, 64%) as a solid. LCMS: (ESI, m/z): 392.3 [M+H]⁺. ¹H NMR (400 MHz, DMSO-d₆) δ ppm 9.54 (s, 1H) 8.55 (d, J=11.51 Hz, 1H) 8.23 (s, 1H) 7.99 (s, 1H) 7.69 (d, J=7.75 Hz, 1H) 6.81 (d, J=7.75 Hz, 1H) 4.93-5.06 (m, 1H) 3.45 (br d, J=12.63 Hz, 2H) 3.07-3.17 (m, 2H) 2.90 (s, 3H) 2.51 (br s, 3H) 2.12-2.22 (m, 2H) 1.98 (br d, J=12.63 Hz, 2H). ¹⁹F NMR (376 MHz, DMSO-d₆) δ ppm−131.661 (s, 1F).

### Example G131: Synthesis of Compound 1305

### Synthesis of Intermediate G273

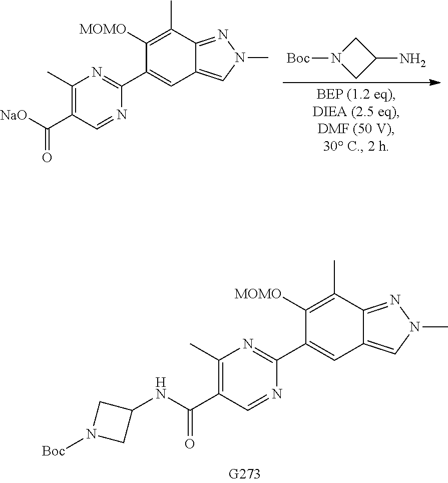

To a mixture of sodium 2-(6-(methoxymethoxy)-2,7-dimethyl-2H-indazol-5-yl)-4-methylpyrimidine-5-carboxylate (100 mg, 273 umol, 1 eq) and tert-butyl 3-aminoazetidine-1-carboxylate (56.5 mg, 328 umol, 1.2 eq) in N,N-dimethylformamide (5.00 mL) was added N,N-diisopropylethylamine (88.4 mg, 684 umol, 119 uL, 2.5 eq) and 2-bromo-1-ethyl-pyridin-1-ium; tetrafluoroborate (89.9 mg, 328 umol, 1.2 eq) at 25° C. The reaction mixture was stirred at 30° C. for 2 h, then diluted with water (10.0 mL) and extracted with ethyl acetate (3×10.0 mL). The organic layers were combined, washed with brine (20.0 mL), dried over Na₂SO₄, filtered, and the filtrate concentrated under reduced pressure to give a residue. The residue was purified by Prep-TLC (ethyl acetate/methanol=10/1) to give tert-butyl 3-[[2-[6-(methoxymethoxy)-2,7-dimethyl-indazol-5-yl]-4-methyl-pyrimidine-5-carbonyl]amino]azetidine-1-carboxylate (130 mg, 74%) as a solid. LCMS (ESI+) m/z: [M+H]⁺ 497.3. ¹H NMR (400 MHz, CDCl₃) δ ppm 8.79 (s, 1H) 7.98 (s, 1H) 7.94 (s, 1H) 7.13 (br d, J=7.09 Hz, 1H) 4.88 (s, 2H) 4.77-4.85 (m, 1H) 4.36 (t, J=8.56 Hz, 2H) 4.23 (s, 3H) 3.90 (dd, J=9.41, 5.14 Hz, 2H) 3.37 (s, 3H) 2.73 (s, 3H) 2.65 (s, 3H) 1.45 (s, 9H).

### Synthesis of Intermediate G274

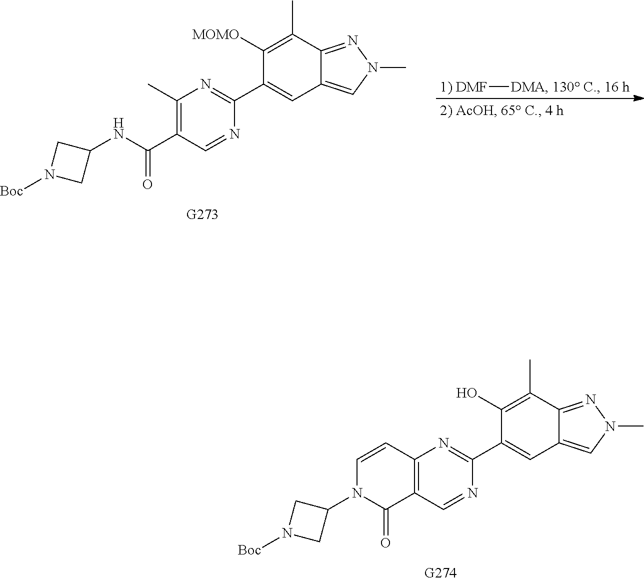

A mixture of tert-butyl 3-[[2-[6-(methoxymethoxy)-2,7-dimethyl-indazol-5-yl]-4-methyl-pyrimidine-5-carbonyl]amino]azetidine-1-carboxylate (110 mg, 221 umol, 1 eq) in DMF-DMA (1.10 mL) was stirred at 130° C. for 16 h, then concentrated under reduced pressure to give a residue. To the residue was added acetic acid (1.00 mL). The reaction mixture was stirred at 65° C. for 4 h, then filtered. The filter cake was washed with methyl tert-butyl ether (3×1.00 mL) and dried to give tert-butyl 3-[2-(6-hydroxy-2,7-dimethyl-indazol-5-yl)-5-oxo-pyrido[4,3-d]pyrimidin-6-yl]azetidine-1-carboxylate (70.0 mg, 40%) as a solid. LCMS (ESI+) m/z: [M+H]⁺ 463.2. ¹H NMR (400 MHz, CDCl₃) δ ppm 12.78 (s, 1H) 10.63 (s, 1H) 9.59 (s, 1H) 9.03 (s, 1H) 8.55 (s, 1H) 8.01 (s, 1H) 5.61 (ddd, J=7.97, 5.03, 3.00 Hz, 1H) 4.53 (dd, J=9.82, 8.32 Hz, 2H) 4.25 (s, 3H) 4.15 (dd, J=10.01, 5.00 Hz, 2H) 2.56 (s, 3H) 1.51 (s, 9H).

### Synthesis of Compound 1305

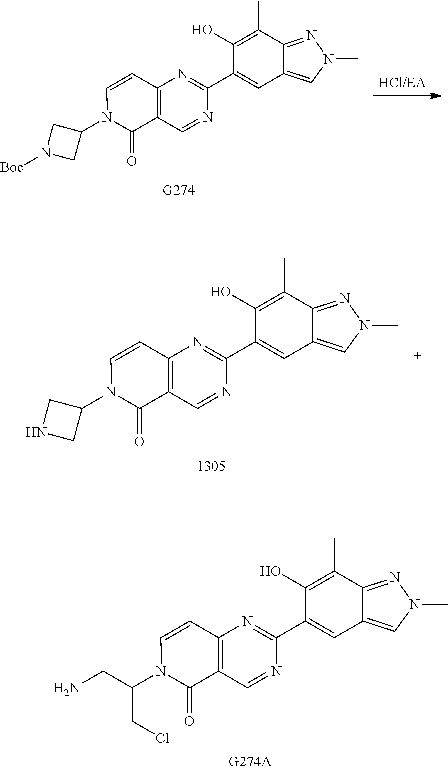 [SMILES: Cc1c(O)c(-c2ncc3c(=O)n(C4CN(C(=O)OC(C)(C)C)C4)ccc3n2)cc2cn(C)nc12 ; Cc1c(O)c(-c2ncc3c(=O)n(C4CNC4)ccc3n2)cc2cn(C)nc12 ; Cc1c(O)c(-c2ncc3c(=O)n(C(CN)CCl)ccc3n2)cc2cn(C)nc12]

To a solution of tert-butyl 3-[2-(6-hydroxy-2,7-dimethyl-indazol-5-yl)-5-oxo-pyrido[4,3-d]pyrimidin-6-yl]azetidine-1-carboxylate (60.0 mg, 97.3 umol, 1 eq) in ethyl acetate (600 uL) was added HCl/ethyl acetate (600 uL, 4N) at 0° C. The reaction mixture was stirred at 25° C. for 1 h, then concentrated under reduced pressure to give a residue. The residue was purified by prep-HPLC (Condition G10, Gradient 1) to give 6-(azetidin-3-yl)-2-(6-hydroxy-2,7-dimethyl-indazol-5-yl)pyrido[4,3-d]pyrimidin-5-one (13.0 mg, 28%) and 6-[1-(aminomethyl)-2-chloro-ethyl]-2-(6-hydroxy-2,7-dimethyl-indazol-5-yl)pyrido[4,3-d]pyrimidin-5-one (3.76 mg, 2%) as solids. 1306: LCMS (ESI+) m/z: [M+H]⁺ 363.1. ¹H NMR (400 MHz, D₂O) δ ppm 8.68 (s, 1H) 7.97 (s, 2H) 7.53 (d, J=7.58 Hz, 1H) 6.32 (d, J=7.58 Hz, 1H) 5.18 (br t, J=8.07 Hz, 1H) 4.53-4.72 (m, 4H) 3.94 (s, 3H) 1.79 (s, 3H). G274A: LCMS (ESI+) m/z: [M+H]⁺ 399.1. ¹H NMR (400 MHz, D₂O) δ ppm 8.89 (s, 1H) 8.09 (s, 1H) 7.96 (s, 1H) 7.88 (d, J=7.75 Hz, 1H) 6.63 (d, J=7.75 Hz, 1H) 5.32-5.40 (m, 1H) 4.11-4.21 (m, 2H) 3.95 (s, 3H) 3.68 (d, J=6.75 Hz, 2H) 1.93 (s, 3H).

### Example G132: Synthesis of Compounds 1417 and 1427

### Synthesis of Intermediate G275

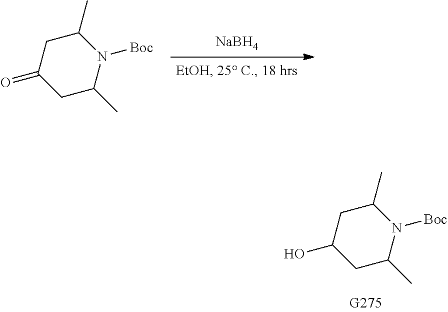 [SMILES: CC1CC(=O)CC(C)N1C(=O)OC(C)(C)C ; "NaBH4" ; CC1CC(O)CC(C)N1C(=O)OC(C)(C)C]

To a solution of tert-butyl 2,6-dimethyl-4-oxo-piperidine-1-carboxylate (13.1 g, 57.6 mmol, 1.0 eq) in ethanol (864 mL) was added sodium borohydride (3.05 g, 80.6 mmol, 1.40 eq) in portions at 0° C. The reaction mixture was stirred at 25° C. for 18 h, then quenched with water (300 mL) slowly at 0° C. under N₂ atmosphere and extracted with dichloromethane (3×200 mL). The organic layer were combined, washed with brine (1.00 L), dried over anhydrous Na₂SO₄, filtered, and the filtrate concentrated in vacuo to give an oil. The oil was purified by silica gel chromatography eluted with petroleum ether/ethyl acetate=3/1 to give tert-butyl 4-hydroxy-2,6-dimethyl-piperidine-1-carboxylate (10.8 g, 47.4 mmol, 82%) as an oil. ¹H NMR (400 MHz, CHLOROFORM-d) δ ppm 4.33-4.15 (m, 2H), 4.04-3.83 (m, 1H), 2.22-2.00 (m, 2H), 1.60 (ddd, J=4.6, 6.7, 13.2 Hz, 3H), 1.47 (s, 9H), 1.41-1.32 (m, 5H), 1.17 (d, J=7.0 Hz, 1H).

### Synthesis of Intermediate G276

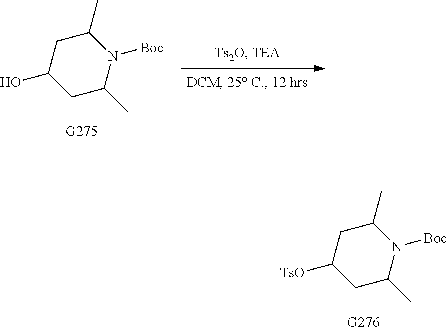 [SMILES: CC1CC(O)CC(C)N1C(=O)OC(C)(C)C ; "Ts2O, TEA" ; Cc1ccc(S(=O)(=O)OC2CC(C)N(C(=O)OC(C)(C)C)C(C)C2)cc1]

To a solution of tert-butyl 4-hydroxy-2,6-dimethyl-piperidine-1-carboxylate (5.0 g, 21.8 mmol, 1.0 eq) in dichloromethane (50.0 mL) was added triethylamine (11.0 g, 109 mmol, 5.0 eq) and p-tolylsulfonyl 4-methylbenzenesulfonate (21.3 g, 65.4 mmol, 3.0 eq) at 0° C. The reaction mixture was stirred at 25° C. for 12 h, then diluted with dichloromethane (100 mL), washed with water (100 mL) and brine (100 mL). The organic layer was dried over Na₂SO₄ and filtered. The filtrate was concentrated under reduced pressure to give an oil, which was purified by column chromatography (SiO₂, petroleum ether/ethyl acetate=10/1 to 1/1) to give tert-butyl 2,6-dimethyl-4-(p-tolylsulfonyloxy)piperidine-1-carboxylate (4.89 g, 10.2 mmol, 47%) as an oil. ¹H NMR (400 MHz, DMSO-d6) δ ppm 7.81 (d, J=8.3 Hz, 2H), 7.53-7.45 (m, 2H), 4.96-4.73 (m, 1H), 4.20-3.75 (m, 2H), 2.43 (s, 3H), 1.96-1.80 (m, 2H), 1.72-1.60 (m, 2H), 1.43-1.34 (m, 9H), 1.24-1.19 (m, 5H), 1.04 (d, J=6.8 Hz, 1H).

### Synthesis of Intermediate G277

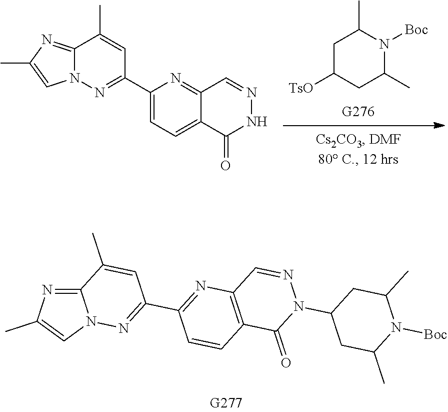 [SMILES: Cc1cn2nc(-c3ccc4c(=O)[nH]ncc4n3)cc(C)c2n1 ; Cc1ccc(S(=O)(=O)OC2CC(C)N(C(=O)OC(C)(C)C)C(C)C2)cc1 ; Cc1cn2nc(-c3ccc4c(=O)n(C5CC(C)N(C(=O)OC(C)(C)C)C(C)C5)ncc4n3)cc(C)c2n1]

To a mixture of 2-(2,8-dimethylimidazo[1,2-b]pyridazin-6-yl)-6H-pyrido[2,3-d]pyridazin-5-one (400 mg, 1.37 mmol, 1.0 eq) and tert-butyl 2,6-dimethyl-4-(p-tolylsulfonyl oxy)piperidine-1-carboxylate (1.31 g, 3.42 mmol, 2.50 eq) in dimethyl formamide (8 mL) was added Cs₂CO₃ (668 mg, 2.05 mmol, 3.0 eq). The reaction mixture was stirred at 100° C. for 1.5 h under N₂ atmosphere, then diluted with water (100 mL) and extracted with ethyl acetate (3×50.0 mL). The organic layers were combined, washed with brine (200 mL), dried over Na₂SO₄, and filtered. The filtrate was concentrated under reduced pressure to give an oil. The oil was purified by silica gel column chromatography (petroleum ether/ethyl acetate=0:1) to afford tert-butyl 4-[2-(2,8-dimethyl-imidazo[1,2-b]pyridazin-6-yl)-5-oxo-pyrido[2,3-d]pyridazin-6-yl]-2,6-dimethyl-piperidine-1-carboxylate (310 mg, 589 umol, 43%) as a solid. LCMS: (ESI, m/z): 504.3 [M+H]⁺. ¹H NMR (400 MHz, METHANOL-d4) δ ppm 8.81-8.71 (m, 2H), 8.64-8.56 (m, 1H), 8.21 (s, 1H), 8.02-7.95 (m, 1H), 5.70-5.48 (m, 1H), 4.63-4.35 (m, 2H), 2.71 (s, 3H), 2.51 (s, 3H), 2.36-2.18 (m, 2H), 2.01-1.82 (m, 2H), 1.51 (s, 9H), 1.44-1.36 (m, 6H).

### Synthesis of Compound 1417

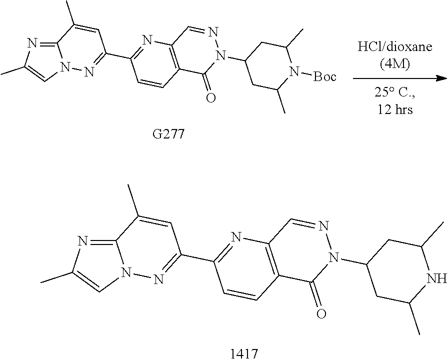

A mixture of tert-butyl 4-[2-(2,8-dimethylimidazo[1,2-b]pyridazin-6-yl)-5-oxo-pyrido[2,3-d]pyridazin-6-yl]-2,6-dimethyl-piperidine-1-carboxylate (310 mg, 616 umol, 1.0 eq) in HCl/dioxane (10.0 mL) was stirred at 25° C. for 12 h, then concentrated under reduced pressure to give a solid, which was triturated with methyl tert-butyl ether (5.0 mL) to give a suspension. The suspension was filtered and the filter cake dried to afford 2-(2,8-dimethylimidazo[1,2-b]pyridazin-6-yl)-6-(2,6-dimethyl-4-piperidyl)pyrido[2,3-d]pyridazin-5-one (230 mg, 571 umol, 92%). LCMS: (ESI, m/z): 404.1 [M+H]⁺. ¹H NMR (400 MHz, METHANOL-d4) δ ppm 8.95-8.90 (m, 1H), 8.86 (s, 1H), 8.84 (d, J=1.2 Hz, 1H), 8.67 (d, J=2.2 Hz, 1H), 8.44 (d, J=0.9 Hz, 1H), 5.61-5.30 (m, 1H), 4.08-3.74 (m, 2H), 2.86 (s, 3H), 2.69 (d, J=0.7 Hz, 3H), 2.47-2.41 (m, 1H), 2.26-1.96 (m, 3H), 1.60 (d, J=7.1 Hz, 1H), 1.44 (dd, J=6.6, 7.6 Hz, 2H), 1.38 (d, J=6.5 Hz, 4H).

### Synthesis of Compound 1427

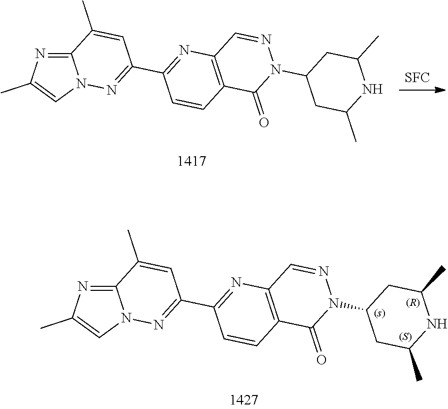

2-(2,8-dimethylimidazo[1,2-b]pyridazin-6-yl)-6-(2,6-dimethyl-4-piperidyl)pyrido[2,3-d]pyridazin-5-one (40 mg, 99.1 umol) was purified by SFC (Condition G3) to give 2-(2,8-dimethylimidazo[1,2-b]pyridazin-6-yl)-6-[(2S,6R)-2,6-dimethyl-4-piperidyl]pyrido[2,3-d]pyridazin-5-one (13.6 mg, 34%). LCMS: (ESI, m/z): 404.1 [M+H]⁺. ¹H NMR (400 MHz, methanol-d4) δ ppm 8.77-8.67 (m, 2H), 8.56 (s, 1H), 8.19 (s, 1H), 7.98 (s, 1H), 5.22 (br t, J=5.2 Hz, 1H), 3.47-3.36 (m, 2H), 2.70 (s, 3H), 2.50 (s, 3H), 2.15 (br d, J=14.4 Hz, 2H), 1.63 (ddd, J=5.4, 11.9, 14.7 Hz, 2H), 1.12 (d, J=6.4 Hz, 6H).

### Example G133: Synthesis of Compounds 1419-1422

### Synthesis of Intermediate G280

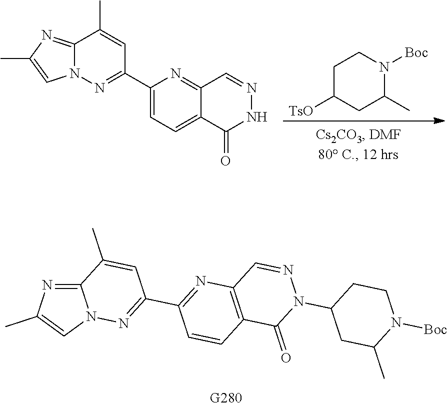 [SMILES: Cc1cn2nc(-c3ccc4c(=O)[nH]ncc4n3)cc(C)c2n1 ; Cc1ccc(S(=O)(=O)OC2CCN(C(=O)OC(C)(C)C)C(C)C2)cc1 ; Cc1cn2nc(-c3ccc4c(=O)n(C5CCN(C(=O)OC(C)(C)C)C(C)C5)ncc4n3)cc(C)c2n1]

To a mixture of 2-(2,8-dimethylimidazo[1,2-b]pyridazin-6-yl)-6H-pyrido[2,3-d]pyridazin-5-one (250 mg, 855 umol, 1.0 eq) and tert-butyl 2-methyl-4-(p-tolylsulfonyloxy) piperidine-1-carboxylate (948 mg, 2.57 mmol, 3.0 eq) in dimethylformamide (6.25 mL) was added Cs₂CO₃ (836 mg, 2.57 mmol, 3.0 eq). The reaction mixture was stirred at 80° C. for 12 h, then diluted with water (20.0 mL) and extracted with ethyl acetate (3×20.0 mL). The organic layers were combined, washed with brine (4×20.0 mL), dried over Na₂SO₄, filtered, and the filtrate concentrated under reduced pressure to give an oil. The oil was purified by column chromatography on silica gel eluted with petroleum ether/ethyl acetate (1/0 to 5/1) to give tert-butyl 4-[2-(2,8-dimethylimidazo[1,2-b]pyridazin-6-yl)-5-oxo-pyrido[2,3-d]pyridazin-6-yl]-2-methyl-piperidine-1-carboxylate (0.20 g, 38%) as a solid. LCMS: (ESI, m/z): 490.3 [M+H]⁺. ¹H NMR (400 MHz, CHLOROFORM-d) δ ppm 8.83-8.77 (m, 1H), 8.74-8.70 (m, 1H), 8.53 (d, J=7.3 Hz, 1H), 8.13 (s, 1H), 7.82 (s, 1H), 5.50-5.22 (m, 1H), 4.29-4.02 (m, 1H), 3.39 (s, 0.5H), 3.24-3.01 (m, 0.5H), 2.79 (s, 3H), 2.58 (s, 3H), 2.26-1.72 (m, 4H), 1.59 (br s, 1H), 1.51 (d, J=3.7 Hz, 9H), 1.35-1.25 (m, 3H).

### Synthesis of Compound 1418

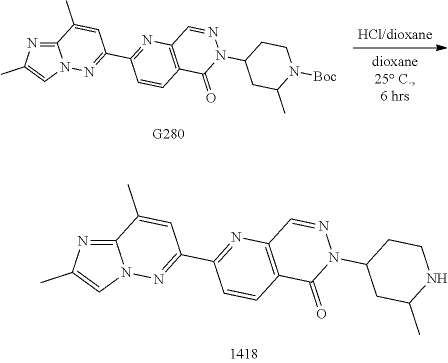 [SMILES: Cc1cn2nc(-c3ccc4c(=O)n(C5CCN(C(=O)OC(C)(C)C)C(C)C5)ncc4n3)cc(C)c2n1 ; Cc1cn2nc(-c3ccc4c(=O)n(C5CCNC(C)C5)ncc4n3)cc(C)c2n1]

To a solution of tert-butyl 4-[2-(2,8-dimethylimidazo[1,2-b]pyridazin-6-yl)-5-oxo-pyrido[2,3-d]pyridazin-6-yl]-2-methyl-piperidine-1-carboxylate (280 mg, 571 umol, 1.0 eq) in dioxane (5.60 mL) was added HCl/dioxane (4.0 M, 5.60 mL). The reaction mixture was stirred at 25° C. for 6 h, then filtered. The filter cake was dried under vacuum to give 2-(2,8-dimethylimidazo[1,2-b]pyridazin-6-yl)-6-(2-methyl-4-piperidyl)pyrido[2,3-d]pyridazin-5-one (280 mg, 91%) as a solid. LCMS: (ESI, m/z): 390.1 [M+H]⁺. ¹H NMR (400 MHz, D₂O) δ ppm 8.84-8.77 (m, 1H), 8.72-8.65 (m, 2H), 8.57 (s, 1H), 8.19 (s, 1H), 5.38-5.25 (m, 1H), 4.05-3.92 (m, 0.5H), 3.66-3.53 (m, 2H), 3.49-3.37 (m, 0.5H), 3.27 (dt, J=2.9, 13.1 Hz, 0.5H), 2.76 (s, 3H), 2.60 (s, 3H), 2.44-2.06 (m, 4H), 1.41 (dd, J=3.0, 6.4 Hz, 3H).

### Synthesis of Compound 1419-1422

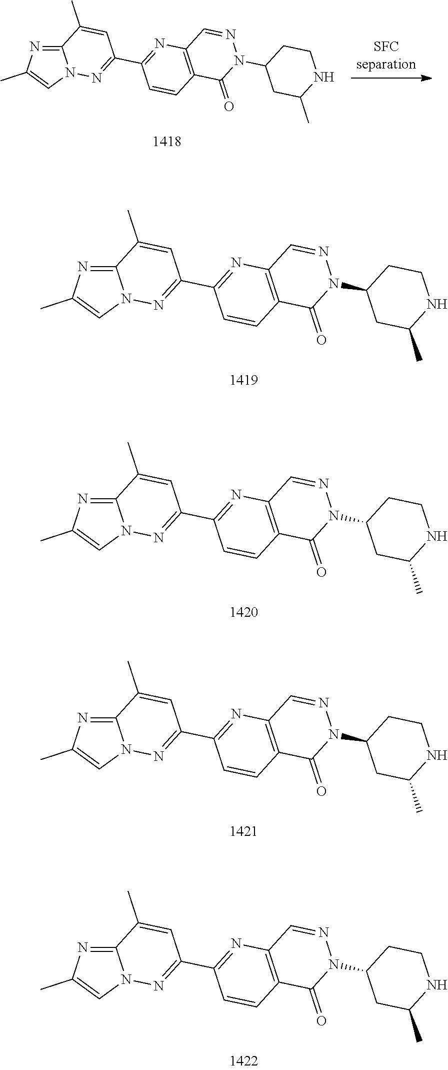 [SMILES: Cc1cn2nc(-c3ccc4c(=O)n(C5CCNC(C)C5)ncc4n3)cc(C)c2n1 ; Cc1cn2nc(-c3ccc4c(=O)n([C@H]5CCN[C@@H](C)C5)ncc4n3)cc(C)c2n1 ; Cc1cn2nc(-c3ccc4c(=O)n([C@@H]5CCN[C@H](C)C5)ncc4n3)cc(C)c2n1 ; Cc1cn2nc(-c3ccc4c(=O)n([C@H]5CCN[C@H](C)C5)ncc4n3)cc(C)c2n1 ; Cc1cn2nc(-c3ccc4c(=O)n([C@@H]5CCN[C@@H](C)C5)ncc4n3)cc(C)c2n1]

2-(2,8-dimethylimidazo[1,2-b]pyridazin-6-yl)-6-(2-methyl-4-piperidyl)pyrido[2,3-d]pyridazin-5-one (280 mg, 571 umol) was separated by SFC (column: Phenomenex-Cellulose-2 (250 mm*30 mm, 10 um); mobile phase: [acetonitrile/methyl alcohol (0.1% NH₃H₂O)]; B %: 60%-60%, 25 min) to give 2-(2,8-dimethylimidazo[1,2-b]pyridazin-6-yl)-6-[(2S,4S)-2-methyl-4-piperidyl]pyrido[2,3-d]pyridazin-5-one (14.2 mg, 6%), 2-(2,8-dimethylimidazo[1,2-b]pyridazin-6-yl)-6-[(2R,4S)-2-methyl-4-piperidyl]pyrido[2,3-d]pyridazin-5-one (19.1 mg, 9%), 2-(2,8-dimethylimidazo[1,2-b]pyridazin-6-yl)-6-[(2R,4R)-2-methyl-4-piperidyl]pyrido[2,3-d]pyridazin-5-one (19.2 mg, 9%), and 2-(2,8-dimethylimidazo[1,2-b]pyridazin-6-yl)-6-[(2S,4R)-2-methyl-4-piperidyl]pyrido[2,3-d]pyridazin-5-one (12.3 mg, 6%) as solids. 1419: LCMS (ESI, m/z): 390.2[M+H]⁺. ¹H NMR (400 MHz, DMSO-d6) δ ppm 8.76 (d, J=8.51 Hz, 1H), 8.74-8.63 (m, 2H), 8.13 (s, 1H), 8.07 (s, 1H), 4.94 (m, 1H), 3.14-3.05 (m, 1H), 2.76-2.63 (m, 5H), 2.41 (s, 3H), 1.82-1.72 (m, 3H), 1.59-1.56 (m, 1H), 1.06 (d, J=6.13 Hz, 3H). 1420: LCMS (ESI, m/z): 390.3[M+H]⁺. ¹H NMR (400 MHz, DMSO-d6) δ ppm 8.80-8.71 (m, 1H), 8.68-8.60 (m, 2H), 8.09 (d, J=19.1 Hz, 2H), 5.21-5.08 (m, 1H), 3.37-3.24 (m, 1H), 2.94-2.85 (m, 2H), 2.63 (s, 3H), 2.41 (s, 3H), 2.09-1.98 (m, 1H), 1.95-1.84 (m, 1H), 1.84-1.74 (m, 1H), 1.65-1.55 (m, 1H), 1.13 (d, J=6.8 Hz, 3H). 1421: LCMS (ESI, m/z): 390.3[M+H]⁺. ¹H NMR (400 MHz, DMSO-d6) δ ppm 8.78-8.73 (m, 1H), 8.70-8.62 (m, 2H), 8.12 (s, 1H), 8.07 (s, 1H), 5.24-5.11 (m, 1H), 3.47-3.38 (m, 2H), 2.63 (s, 3H), 2.41 (s, 3H), 2.17-1.92 (m, 4H), 1.28 (d, J=6.3 Hz, 3H). 1422: LCMS (ESI, m/z): 390.2[M+H]⁺. ¹H NMR (400 MHz, DMSO-d6) δ ppm 8.77 (d, J=8.4 Hz, 1H), 8.71-8.59 (m, 2H), 8.18 (s, 1H), 8.07 (d, J=1.0 Hz, 1H), 5.21 (td, J=4.6, 9.0 Hz, 1H), 3.37-3.33 (m, 1H), 2.99-2.85 (m, 2H), 2.67 (s, 3H), 2.44 (s, 3H), 2.09-1.98 (m, 1H), 1.95-1.83 (m, 1H), 1.82-1.73 (m, 1H), 1.60 (td, J=4.2, 12.6 Hz, 1H), 1.15 (d, J=6.7 Hz, 3H).

### Example G134: Synthesis of Compound 1528

### Synthesis of Compound 1528

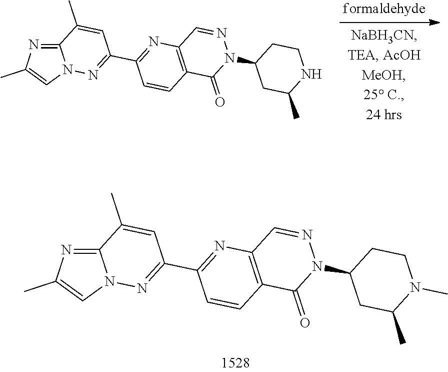

To a solution of 2-(2,8-dimethylimidazo[1,2-b]pyridazin-6-yl)-6-[(2R,4S)-2-methyl-4-piperidyl]pyrido[2,3-d]pyridazin-5-one (10.0 mg, 25.7 umol, 1.0 eq) in methanol (0.20 mL) was added triethylamine (5.20 mg, 51.3 umol, 2.0 eq). The reaction mixture was stirred at 25° C. for 10 min. To the reaction mixture was added acetic acid (5.87 uL, 5.0 eq) and formaldehyde (6.17 mg, 205 umol, 8.0 eq) and the resulting mixture was stirred at 25° C. for an additional 10 min. Sodium cyanoborohydride (2.42 mg, 77.0 umol, 1.50 eq) was added to the reaction mixture. The reaction mixture was stirred at 25° C. for 24 h, then diluted with water (5.0 mL) and extracted with dichloromethane (3×4.0 mL). The organic layers were combined, dried over Na₂SO₄, filtered, and the filtrate concentrated under reduced pressure to give a residue. The residue was triturated with dimethylformamide for 15 min to give a suspension. The suspension was filtered. The filter cake was dried under vacuum to give 2-(2,8-dimethylimidazo[1,2-b]pyridazin-6-yl)-6-(1,2-dimethyl-4-piperidyl)pyrido[2,3-d]pyridazin-5-one (4.25 mg, 29%) was as a solid. LCMS: (ESI, m/z): 404.3 [M+H]⁺. ¹H NMR (400 MHz, DMSO-d₆) δ ppm 8.79 (d, J=8.5 Hz, 1H), 8.75-8.58 (m, 2H), 8.19 (s, 1H), 8.08 (d, J=1.0 Hz, 1H), 4.98-4.86 (m, 1H), 2.97-2.90 (m, 1H), 2.67 (s, 3H), 2.44 (s, 3H), 2.22 (br s, 3H), 2.10-1.97 (m, 2H), 1.84-1.69 (m, 3H), 1.25 (s, 1H), 1.08 (d, J=6.1 Hz, 3H).

### Example G135: Synthesis of Compound 1529

### Synthesis of Compound 1529

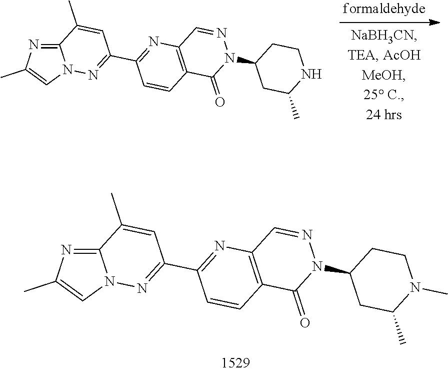

To a solution of 2-(2,8-dimethylimidazo[1,2-b]pyridazin-6-yl)-6-[(2R,4S)-2-methyl-4-piperidyl]pyrido[2,3-d]pyridazin-5-one (10.0 mg, 25.7 umol, 1.0 eq) in methanol (0.20 mL) was added triethylamine (5.20 mg, 51.3 umol, 2.0 eq). The reaction mixture was stirred at 25° C. for 10 min. Formaldehyde (6.17 mg, 205 umol, 8.0 eq) and acetic acid (5.87 uL, 5.0 eq) were added to the reaction mixture and the resulting mixture was stirred at 25° C. for 10 min. Sodium cyanoborohydride (2.42 mg, 77.0 umol, 1.50 eq) was added to the reaction mixture. The reaction mixture was stirred at 25° C. for 24 h, then diluted with water (5.0 mL) and extracted with dichloromethane (3×4.0 mL). The organic layers were combined, dried over Na₂SO₄, filtered, and the filtrate concentrated under reduced pressure to residue. The residue was triturated with methanol (0.50 mL) for 15 min to give a suspension. The suspension was filtered. The filter cake was dried under vacuum to give 2-(2,8-dimethylimidazo[1,2-b]pyridazin-6-yl)-6-[(2R,4S)-1,2-dimethyl-4-piperidyl]pyrido[2,3-d]pyridazin-5-one (2.15 mg, 21%) as a solid. LCMS: (ESI, m/z): 404.2 [M+H]⁺. ¹H NMR (400 MHz, DMSO-d6) δ ppm 8.77 (d, J=8.5 Hz, 1H), 8.72-8.59 (m, 2H), 8.19 (s, 1H), 8.08 (d, J=1.0 Hz, 1H), 5.21-5.02 (m, 1H), 3.14-3.07 (m, 1H), 2.67 (d, J=0.6 Hz, 3H), 2.62-2.57 (m, 1H), 2.44 (s, 3H), 2.28 (s, 3H), 2.22-2.14 (m, 1H), 2.04 (br dd, J=4.6, 12.0 Hz, 1H), 1.77 (br dd, J=1.8, 12.6 Hz, 1H), 1.66 (br d, J=13.0 Hz, 1H), 1.23 (s, 1H), 1.07 (d, J=6.8 Hz, 3H).

### Example G136: Synthesis of Compounds 1536-1538

### Synthesis of Compound 1536, 1537, 1538

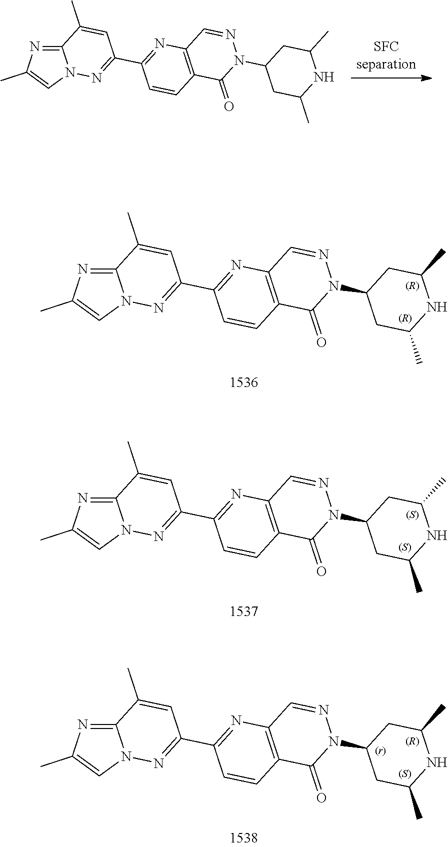

21-1 (310 mg, 768 umol) was separated by SFC (column: DAICEL CHIRALCEL OX (250 mm*30 mm, 10 um); mobile phase: [0.1% NH₃H₂O in ethanol]; B %: 60%-60%, 15 min, column: DAICEL CHIRALPAK IG (250 mm*30 mm, 10 um) to give 2-(2,8-dimethylimidazo[1,2-b]pyridazin-6-yl)-6-[(2S,6R)-2,6-dimethyl-4-piperidyl]pyrido[2,3-d]pyridazin-5-one (80.0 mg, 26%), 2-(2,8-dimethylimidazo[1,2-b]pyridazin-6-yl)-6-[(2R,6R)-2,6-dimethyl-4-piperidyl]pyrido[2,3-d]pyridazin-5-one (15.0 mg, 5%), and 2-(2,8-dimethyl imidazo[1,2-b]pyridazin-6-yl)-6-[(2S,6S)-2,6-dimethyl-4-piperidyl]pyrido[2,3-d]pyridazin-5-one (20.0 mg, 6%) as solids. LCMS of 1536: (ESI, m/z): 404.2 [M+H]⁺. ¹H NMR (400 MHz, methanol-d4) δ=8.81-8.70 (m, 2H), 8.59 (s, 1H), 8.21 (d, J=1.0 Hz, 1H), 8.00 (s, 1H), 5.43 (tt, J=3.9, 12.1 Hz, 1H), 3.65-3.54 (m, 1H), 3.29-3.18 (m, 1H), 2.71 (d, J=0.8 Hz, 3H), 2.51 (s, 3H), 2.26 (dt, J=5.2, 12.5 Hz, 1H), 1.95 (br d, J=12.1 Hz, 1H), 1.85-1.72 (m, 2H), 1.41 (d, J=7.0 Hz, 3H), 1.19 (d, J=6.3 Hz, 3H). LCMS of 1537: (ESI, m/z): 404.2 [M+H]⁺. ¹H NMR (400 MHz, methanol-d4) δ=8.81-8.73 (m, 2H), 8.60 (s, 1H), 8.23 (d, J=1.1 Hz, 1H), 8.00 (d, J=0.7 Hz, 1H), 5.44 (tt, J=3.9, 12.2 Hz, 1H), 3.59 (quin, J=6.3 Hz, 1H), 3.28 (br s, 1H), 2.72 (d, J=1.0 Hz, 3H), 2.51 (s, 3H), 2.26 (dt, J=5.3, 12.6 Hz, 1H), 1.95 (br d, J=12.4 Hz, 1H), 1.83-1.75 (m, 2H), 1.41 (d, J=7.0 Hz, 3H), 1.19 (d, J=6.2 Hz, 3H). LCMS of 1538: (ESI, m/z): 404.2 [M+H]⁺. ¹H NMR (400 MHz, methanol-d4) δ=8.80-8.71 (m, 2H), 8.59 (s, 1H), 8.22 (d, J=1.1 Hz, 1H), 7.99 (s, 1H), 5.14 (tt, J=4.1, 11.8 Hz, 1H), 3.03-2.89 (m, 2H), 2.71 (d, J=0.8 Hz, 3H), 2.51 (s, 3H), 1.96-1.86 (m, 2H), 1.73 (q, J=11.9 Hz, 2H), 1.22 (d, J=6.3 Hz, 6H).

### Example G137: Synthesis of Compound 1541

### Synthesis of Intermediate G281

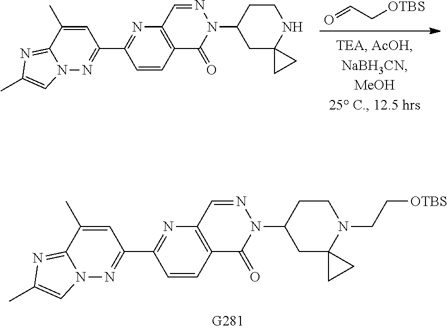 [SMILES: Cc1cn2nc(-c3ccc4c(=O)n(C5CCNC6(CC6)C5)ncc4n3)cc(C)c2n1 ; CC(C)(C)[Si](C)(C)OCC=O ; Cc1cn2nc(-c3ccc4c(=O)n(C5CCN(CCO[Si](C)(C)C(C)(C)C)C6(CC6)C5)ncc4n3)cc(C)c2n1]

To a mixture of 6-(4-azaspiro[2.5]octan-7-yl)-2-(2,8-dimethylimidazo[1,2-b]pyridazin-6-yl)pyrido[2,3-d]pyridazin-5-one (40.0 mg, 99.6 umol, 1.00 eq) in methanol (0.40 mL) was added triethylamine (20.1 mg, 199 umol, 27.7 uL, 2.00 eq). The reaction mixture was stirred at 25° C. for 30 min. To the reaction mixture was added 2-[tert-butyl(dimethyl)silyl]oxyacetaldehyde (34.7 mg, 199 umol, 37.9 uL, 2.00 eq), followed by acetic acid (5.98 mg, 99.6 umol, 5.70 uL, 1.00 eq), then sodium cyanotrihydroborate (9.39 mg, 149 umol, 1.50 eq). The reaction mixture was stirred at 25° C. for 12 h, then diluted with water (50.0 mL) and extracted with ethyl acetate (3×20.0 mL). The organic layers were combined, dried over Na₂SO₄, filtered, and the filtrate concentrated under reduced pressure to give 6-[4-[2-[tert-butyl(dimethyl)silyl]oxyethyl]-4-azaspiro[2.5]octan-7-yl]-2-(2,8-dimethylimidazo[1,2b]pyridazin-6-yl)pyrido[2,3-d]pyridazin-5-one (42.0 mg, 54%) as a solid. LCMS: (ESI, m/z): 560.0 [M+H]⁺. ¹H NMR (400 MHz, CHLOROFORM-d) δ ppm 8.78-8.83 (m, 1H) 8.70-8.77 (m, 1H) 8.52-8.56 (m, 1H) 8.14 (d, J=0.95 Hz, 1H) 7.82 (s, 1H) 5.32-5.42 (m, 1H) 3.70-3.74 (m, 5H) 3.63-3.68 (m, 5H) 2.79 (s, 3H) 2.57 (s, 3H) 0.92 (s, 9H) 0.12 (s, 4H) 0.08-0.10 (m, 6H).

### Synthesis of Compound 1541

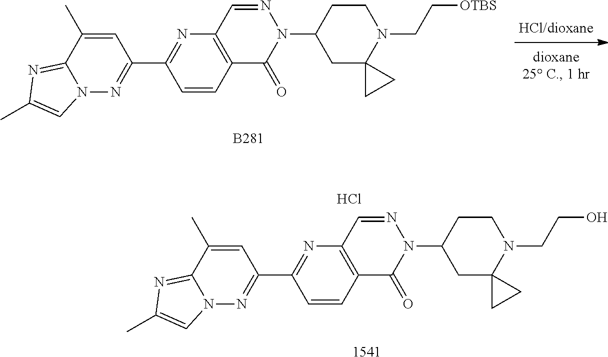 [SMILES: Cc1cn2nc(-c3ccc4c(=O)n(C5CCN(CCO[Si](C)(C)C(C)(C)C)C6(CC6)C5)ncc4n3)cc(C)c2n1 ; "B281" ; Cl ; Cc1cn2nc(-c3ccc4c(=O)n(C5CCN(CCO)C6(CC6)C5)ncc4n3)cc(C)c2n1 ; "1541"]

To a solution of 6-[4-[2-[tert-butyl(dimethyl)silyl]oxyethyl]-4-azaspiro[2.5]octan-7-yl]-2-(2,8-dimethylimidazo[1,2-b]pyridazin-6-yl)pyrido[2,3-d]pyridazin-5-one (40.0 mg, 71.4 umol, 1.00 eq) in dioxane (1.00 mL) was added HCl/dioxane (8.00 M, 803 uL, 90.0 eq). The reaction mixture was stirred at 25° C. for 1 h, then filtered. The filter cake was purified by prep-HPLC (column: Phenomenex Luna 80*30 mm*3 um; mobile phase: [water (HCl)-acetonitrile]; B %: 1%-30%, 8 min) to afford 2-(2,8-dimethylimidazo[1,2-b]pyridazin-6-yl)-6-[4-(2-hydroxyethyl)-4-azaspiro[2.5]octan-7-yl]pyrido-[2,3-d]pyridazin-5-one (5.89 mg, 19%) as a solid. LCMS: (ESI, m/z): 446.2[M+H]⁺. ¹H NMR (400 MHz, METHANOL-d4) δ ppm 8.91-8.95 (m, 1H) 8.85-8.89 (m, 1H) 8.82 (d, J=0.95 Hz, 1H) 8.69 (s, 1H) 8.43 (d, J=0.95 Hz, 1H) 5.36-5.52 (m, 1H) 3.87-4.01 (m, 2H) 3.62-3.80 (m, 4H) 2.94-3.04 (m, 1H) 2.86 (d, J=0.83 Hz, 3H) 2.69 (d, J=0.60 Hz, 4H) 2.16-2.26 (m, 1H) 1.48-1.56 (m, 1H) 1.33-1.48 (m, 2H) 1.01-1.16 (m, 2H).

### Example G138: Synthesis of Compounds 1545 and 1456

### Synthesis of Intermediate G282

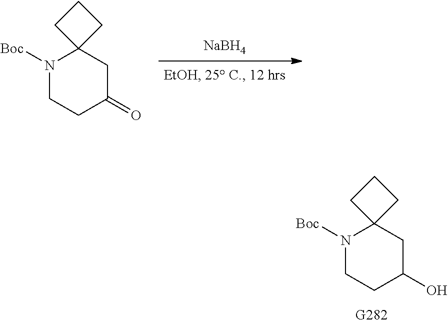 [SMILES: CC(C)(C)OC(=O)N1CCC(=O)CC12CCC2 ; "NaBH4" ; CC(C)(C)OC(=O)N1CCC(O)CC12CCC2]

To a solution of tert-butyl 8-oxo-5-azaspiro[3.5]nonane-5-carboxylate (4.00 g, 16.7 mmol, 1.00 eq) in ethanol (264 mL) was added sodium borohydride (885 mg, 23.4 mmol, 1.40 eq) in portions at 0° C. The reaction mixture was stirred at 25° C. for 12 hr, then quenched with water (100 mL) slowly at 0° C. under N₂ atmosphere and extracted with dichloromethane (3×100 mL). The organic layers were combined, washed with brine (300 mL), dried over Na₂SO₄, filtered and the filtrate concentrated in vacuum to give an oil. The oil was purified by silica gel chromatography eluted with petroleum ether/ethyl acetate=3/1 to give tert-butyl 8-hydroxy-5-azaspiro[3.5]nonane-5-carboxylate (4.00 g, 16.5 mmol, 99%) as an oil. ¹H NMR (400 MHz, CHLOROFORM-d) δ=3.93 (tt, J=5.0, 10.3 Hz, 1H), 3.83 (td, J=3.4, 13.9 Hz, 1H), 2.70-2.55 (m, 2H), 2.23 (dd, J=4.6, 12.5 Hz, 1H), 2.15-2.05 (m, 1H), 2.03-1.89 (m, 2H), 1.87-1.78 (m, 1H), 1.75-1.65 (m, 3H), 1.48 (br d, J=12.0 Hz, 1H), 1.44 (s, 9H), 1.32-1.21 (m, 1H).

### Synthesis of Intermediate G283

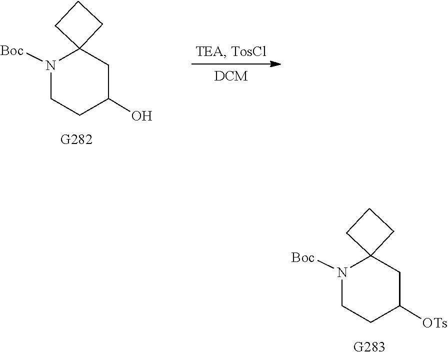 [SMILES: CC(C)(C)OC(=O)N1CCC(O)CC12CCC2 ; Cc1ccc(S(=O)(=O)OC2CCN(C(=O)OC(C)(C)C)C3(CCC3)C2)cc1]

To a solution of tert-butyl 8-hydroxy-5-azaspiro[3.5]nonane-5-carboxylate (3.00 g, 12.4 mmol, 1.00 eq) in dichloromethane (60.0 mL) was added triethylamine (6.30 g, 62.1 mmol, 8.64 mL, 5.00 eq) and p-tolylsulfonyl 4-methylbenzenesulfonate (12.1 g, 37.2 mmol, 3.00 eq) at 0° C. The reaction mixture was stirred at 25° C. for 12 h, then diluted with dichloromethane (100 mL), and washed with water (100 mL) and brine (100 mL). The organic layer was dried over Na₂SO₄ and filtered. The filtrate was concentrated under reduced pressure to give an oil. The oil was purified by column chromatography (SiO₂, petroleum ether/ethyl acetate=10/1 to 1/1) to give tert-butyl 8-(p-tolylsulfonyloxy)-5-azaspiro[3.5]nonane-5-carboxylate (3.00 g, 7.59 mmol, 61%) as an oil. ¹HNMR (400 MHz, CHLOROFORM-d) δ=7.81 (d, J=8.4 Hz, 2H), 7.36 (d, J=8.0 Hz, 2H), 4.81-4.69 (m, 1H), 3.73 (td, J=4.1, 14.1 Hz, 1H), 2.71 (ddd, J=2.8, 11.3, 14.1 Hz, 1H), 2.58-2.48 (m, 1H), 2.47 (s, 3H), 2.17-2.04 (m, 2H), 2.03-1.96 (m, 1H), 1.96-1.87 (m, 1H), 1.80-1.71 (m, 2H), 1.71-1.63 (m, 1H), 1.62-1.58 (m, 1H), 1.54-1.46 (m, 1H), 1.43 (s, 9H).

### Synthesis of Intermediate G284

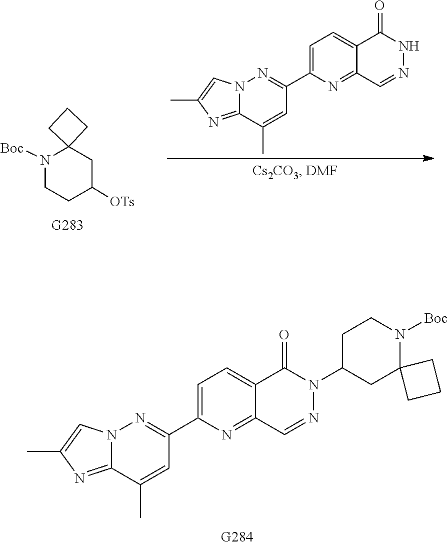 [SMILES: Cc1ccc(S(=O)(=O)OC2CCN(C(=O)OC(C)(C)C)C3(CCC3)C2)cc1 ; Cc1cn2nc(-c3ccc4c(=O)[nH]ncc4n3)cc(C)c2n1 ; Cc1cn2nc(-c3ccc4c(=O)n(C5CCN(C(=O)OC(C)(C)C)C6(CCC6)C5)ncc4n3)cc(C)c2n1]

A suspension of 2-(2,8-dimethylimidazo[1,2-b]pyridazin-6-yl)-6H-pyrido[2,3-d]pyridazin-5-one (400 mg, 1.37 mmol, 1.00 eq), tert-butyl 8-(p-tolylsulfonyloxy)-5-azaspiro[3.5]nonane-5-carboxylate (811 mg, 2.05 mmol, 1.50 eq), and Cs₂CO₃ (1.34 g, 4.11 mmol, 3.00 eq) in dimethyl formamide (4.00 mL) was stirred at 80° C. for 24 h, then diluted with water (20.0 mL) and extracted with ethyl acetate (3×10.0 mL). The organic layers were combined, washed with brine (30.0 mL), dried over Na₂SO₄, and filtered. The filtrate was concentrated under reduced pressure to give an oil. The oil was purified by silica gel column chromatography (petroleum ether/ethyl acetate=0/1) to afford tert-butyl 8-[2-(2,8-dimethylimidazo[1,2-b]pyridazin-6-yl)-5-oxo-pyrido[2,3-d]pyridazin-6-yl]-5-azaspiro[3.5]nonane-5-carboxylate (150 mg, 290 umol, 21%) as a solid. LCMS: (ESI, m/z): 516.2 [M+H]⁺. ¹H NMR (400 MHz, METHANOL-d4) δ=8.78-8.69 (m, 2H), 8.59 (s, 1H), 8.19 (d, J=1.1 Hz, 1H), 7.98 (s, 1H), 5.50-5.37 (m, 1H), 4.03-3.93 (m, 1H), 2.92 (dt, J=2.3, 13.4 Hz, 1H), 2.77-2.66 (m, 4H), 2.50 (s, 3H), 2.41-2.32 (m, 1H), 2.24-2.04 (m, 4H), 2.03-1.91 (m, 1H), 1.84-1.72 (m, 3H), 1.51 (s, 9H).

### Synthesis of Compound 1546

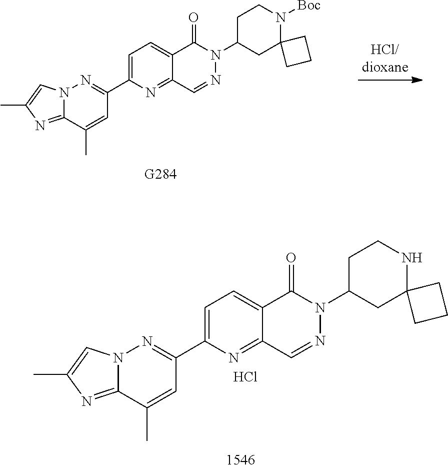 [SMILES: Cc1cn2nc(-c3ccc4c(=O)n(C5CCN(C(=O)OC(C)(C)C)C6(CCC6)C5)ncc4n3)cc(C)c2n1 ; Cc1cn2nc(-c3ccc4c(=O)n(C5CCNC6(CCC6)C5)ncc4n3)cc(C)c2n1 ; Cl]

A mixture of tert-butyl 8-[2-(2,8-dimethylimidazo[1,2-b]pyridazin-6-yl)-5-oxo-pyrido[2,3-d]pyridazin-6-yl]-5-azaspiro[3.5]nonane-5-carboxylate (150 mg, 290 umol, 1.00 eq) in HCl/dioxane (2.00 M, 5.8 mL) was stirred at 25° C. for 12 h. The reaction mixture was concentrated under reduced pressure to give 2-(2,8-dimethylimidazo[1,2-b]pyridazin-6-yl)-6-(5-azaspiro[3.5]nonan-8-yl)pyrido[2,3-d]pyridazin-5(6H)-one hydrochloride (120 mg, 288 umol, 99%) as a solid. LCMS: (ESI, m/z): 416.2 [M+H]⁺. ¹HNMR (400 MHz, METHANOL-d4) δ=8.93-8.91 (m, 1H), 8.87-8.85 (m, 1H) 8.73 (d, J=1.1 Hz, 1H), 8.67 (s, 1H), 8.37 (s, J=1.0 Hz, 1H), 5.30 (tt, J=3.9, 11.8 Hz, 1H), 3.55-3.46 (m, 1H), 3.28-3.21 (m, 1H), 2.86 (d, J=1.0 Hz, 3H), 2.69 (d, J=0.8 Hz, 3H), 2.54-2.27 (m, 6H), 2.23-2.02 (m, 4H).

### Synthesis of Compound 1544, 1545

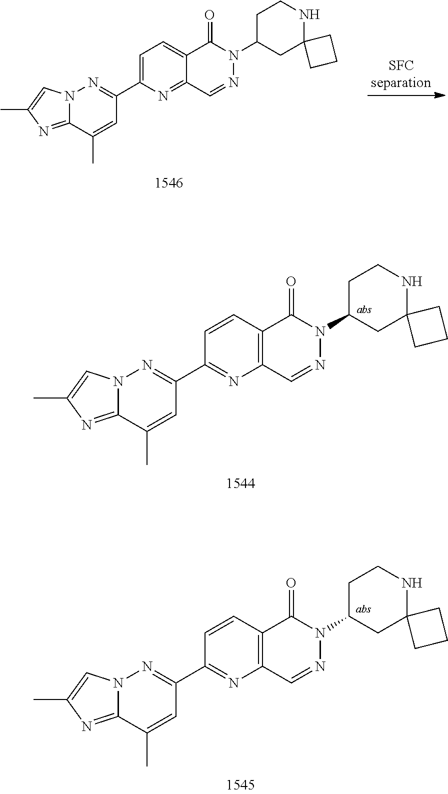 [SMILES: Cc1cn2nc(-c3ccc4c(=O)n(C5CCNC6(CCC6)C5)ncc4n3)cc(C)c2n1 ; Cc1cn2nc(-c3ccc4c(=O)n([C@H]5CCNC6(CCC6)C5)ncc4n3)cc(C)c2n1 ; Cc1cn2nc(-c3ccc4c(=O)n([C@@H]5CCNC6(CCC6)C5)ncc4n3)cc(C)c2n1]

2-(2,8-dimethylimidazo[1,2-b]pyridazin-6-yl)-6-(5-azaspiro[3.5]nonan-8-yl)pyrido[2,3-d]pyridazin-5(6H)-one hydrochloride (98 mg, 235 umol) was separated by SFC (column: DAICEL CHIRALCEL OJ (250 mm*30 mm, 10 um); mobile phase: [0.1% NH₃H₂O ethanol]; B %: 15%-35%, 12 min) to give 6-[(8S)-5-azaspiro[3.5]nonan-8-yl]-2-(2,8-dimethylimidazo[1,2-b]pyridazin-6-yl)pyrido[2,3-d]pyridazin-5-one (28 mg, 66.72 umol, 28%) and 6-[(8R)-5-azaspiro[3.5]nonan-8-yl]-2-(2,8-dimethylimidazo[1,2-b]pyridazin-6-yl)pyrido[2,3-d]pyridazin-5-one (31 mg, 73.12 umol, 31%). LCMS for 1544: (ESI, m/z): 416.2 [M+H]⁺. ¹H NMR (400 MHz, METHANOL-d4) δ ppm 8.63-8.67 (m, 1H) 8.58-8.62 (m, 1H) 8.52 (s, 1H) 8.08 (d, J=0.88 Hz, 1H) 7.90 (s, 1H) 5.11 (tt, J=11.88, 3.81 Hz, 1H) 2.96-3.07 (m, 1H) 2.88 (td, J=12.54, 2.44 Hz, 1H) 2.64 (s, 3H) 2.47 (s, 3H) 2.24-2.35 (m, 1H) 2.18 (br dd, J=12.32, 1.69 Hz, 1H) 1.95-2.13 (m, 5H) 1.82-1.94 (m, 3H). LCMS for 1545: (ESI, m/z): 416.2 [M+H]⁺. ¹H NMR (400 MHz, METHANOL-d4) δ ppm 8.68 (q, J=8.63 Hz, 2H) 8.55 (s, 1H) 8.14 (d, J=1.13 Hz, 1H) 7.94 (d, J=0.63 Hz, 1H) 5.13 (tt, J=11.91, 3.97 Hz, 1H) 3.01 (dq, J=12.48, 2.22 Hz, 1H) 2.88 (td, J=12.57, 2.63 Hz, 1H) 2.67 (d, J=0.88 Hz, 3H) 2.49 (s, 3H) 2.28 (qd, J=7.55, 3.88 Hz, 1H) 2.14-2.21 (m, 1H) 1.95-2.12 (m, 5H) 1.79-1.92 (m, 3H).

### Example G139: Synthesis of Compound 1429

### Synthesis of Intermediate G285

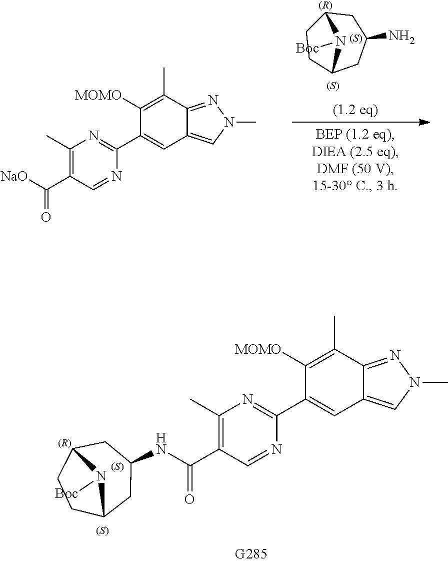

To a mixture of sodium 2-(6-(methoxymethoxy)-2,7-dimethyl-2H-indazol-5-yl)-4-methylpyrimidine-5-carboxylate (250 mg, 684 umol, 1 eq) and tert-butyl (1S,5R)-3-amino-8-azabicyclo[3.2.1]octane-8-carboxylate (185 mg, 821 umol, 1.2 eq) in DMF (12.5 mL) was added DIPEA (221 mg, 1.71 mmol, 297 uL, 2.5 eq) and 2-bromo-1-ethyl-pyridin-1-ium; tetrafluoroborate (224 mg, 821 umol, 1.2 eq) at 15° C. The reaction mixture was stirred at 30° C. for 3 h under nitrogen atmosphere, then diluted with water (30.0 mL) and extracted with ethyl acetate (2×30.0 mL) and dichloromethane (30.0 mL). The organic layers were combined, washed with brine (90.0 mL), dried over with Na₂SO₄, filtered, and the filtrate concentrated under reduced pressure to give a residue. The residue was purified by silica gel chromatography (dichloromethane:methanol=19:1) to give tert-butyl (1S,5R)-3-[[2-[6-(methoxymethoxy)-2,7-dimethyl-indazol-5-yl]-4-methyl-pyrimidine-5-carbonyl]amino]-8-azabicyclo[3.2.1]octane-8-carboxylate (290 mg, 77%) as a solid. LCMS: (ESI, m/z): 551.3 [M+H]⁺. ¹H NMR (400 MHz, CHLOROFORM-d) δ ppm 8.73 (s, 1H) 7.96 (d, J=14.92 Hz, 2H) 5.83 (br d, J=8.31 Hz, 1H) 4.89 (s, 2H) 4.51-4.67 (m, 1H) 4.20-4.41 (m, 5H) 3.39 (s, 3H) 2.73 (s, 3H) 2.65 (s, 3H) 2.02-2.09 (m, 4H) 1.80-1.90 (m, 2H) 1.61-1.74 (m, 2H) 1.48 (s, 9H).

### Synthesis of Intermediate G286

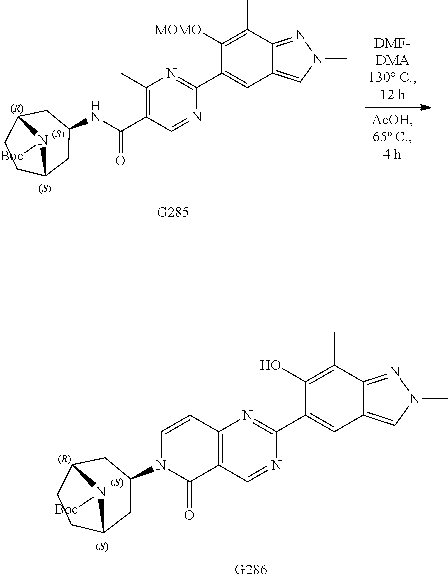

A mixture of tert-butyl (1S,5R)-3-[[2-[6-(methoxymethoxy)-2,7-dimethyl-indazol-5-yl]-4-methyl-pyrimidine-5-carbonyl]amino]-8-azabicyclo[3.2.1]octane-8-carboxylate (290 mg, 526 umol, 1 eq) in DMF-DMA (2.90 mL) was stirred at 130° C. for 12 h under N₂ atmosphere, then concentrated under reduced pressure to give a residue. The residue was dissolved in acetic acid (2.90 mL), then stirred at 65° C. for 4 h. The resulting mixture was cooled to 15° C. A precipitate formed that was collected by filtration and triturated with methyl tert-butyl ether. The resulting suspension was filtered and the solid dried to afford tert-butyl (1S,5R)-3-[2-(6-hydroxy-2,7-dimethyl-indazol-5-yl)-5-oxo-pyrido[4,3-d]pyrimidin-6-yl]-8-azabicyclo[3.2.1]octane-8-carboxylate (32-3, 130 mg, 40%) as a solid LCMS: (ESI, m/z): 517.3 [M+H]⁺. ¹H NMR (400 MHz, CHLOROFORM-d) δ ppm 13.16 (s, 1H) 9.65 (s, 1H) 9.04 (s, 1H) 7.99 (s, 1H) 7.47 (d, J=7.70 Hz, 1H) 6.69 (d, J=7.70 Hz, 1H) 5.57-5.65 (m, 1H) 4.37-4.50 (m, 2H) 4.23 (s, 3H) 2.57 (s, 3H) 2.09-2.14 (m, 2H) 1.85-1.90 (m, 2H) 1.58 (br s, 4H) 1.54 (s, 9H).

### Synthesis of Compound 1429

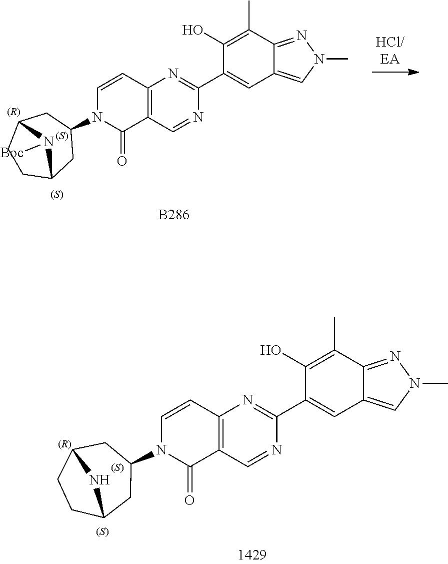

To a solution of tert-butyl (1S,5R)-3-[2-(6-hydroxy-2,7-dimethyl-indazol-5-yl)-5-oxo-pyrido[4,3-d]pyrimidin-6-yl]-8-azabicyclo[3.2.1]octane-8-carboxylate (32-3, 75.0 mg, 145 umol, 1 eq) in ethyl acetate (750 uL) was added HCl in ethyl acetate (4 M, 750 uL). The reaction mixture was stirred at 15° C. for 12 h, then concentrated under reduced pressure to give a solid. The resulting solid was dissolved in dichloromethane, then concentrated under reduced pressure to give a residue. The residue was triturated with dichloromethane and filtered to collect the resulting solid. The filter cake was dried in vacuum to afford 6-[(1S,5R)-8-azabicyclo[3.2.1]octan-3-yl]-2-(6-hydroxy-2,7-dimethyl-indazol-5-yl)pyrido[4,3-d]pyrimidin-5-one (60.4 mg, 84%) as a solid. LCMS: (ESI, m/z): 417.6 [M+H]⁺. ¹H NMR (400 MHz, METHANOL-d₄) δ ppm 9.65 (s, 1H) 9.21 (s, 1H) 8.71 (s, 1H) 7.98 (d, J=7.75 Hz, 1H) 6.87 (d, J=7.75 Hz, 1H) 5.29-5.40 (m, 1H) 4.26-4.33 (m, 5H) 2.44-2.52 (m, 5H) 2.26 (s, 4H) 2.10-2.17 (m, 2H).

### Example G140: Synthesis of Compound 1431

### Synthesis of Intermediate G287

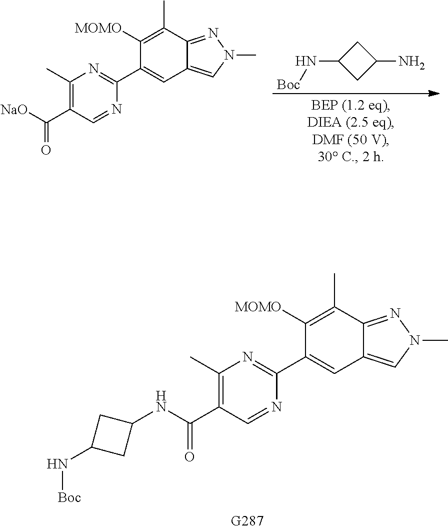

To a mixture of sodium 2-(6-(methoxymethoxy)-2,7-dimethyl-2H-indazol-5-yl)-4-methylpyrimidine-5-carboxylate (200 mg, 547 umol, 1 eq) and tert-butyl N-(3-aminocyclobutyl)carbamate (122.3 mg, 656 umol, 1.2 eq) in N,N-dimethylformamide (10.0 mL) was added N,N-diisopropylethylamine (176 mg, 1.37 mmol, 238 uL, 2.5 eq) and 2-bromo-1-ethyl-pyridin-1-ium; tetrafluoroborate (179 mg, 656 umol, 1.2 eq) at 15° C. The reaction mixture was stirred at 30° C. for 2 h under N₂ atmosphere. The reaction mixture was diluted with water (10.0 mL) and extracted with ethyl acetate (3×10.0 mL). The organic layers were combined, washed with brine (20.0 mL), dried over Na₂SO₄, filtered, and the filtrate concentrated under reduced pressure to give a residue. The residue was purified by silica gel chromatography (dichloromethane/methanol=24/1) to give tert-butyl N-[3-[[2-[6-(methoxymethoxy)-2,7-dimethyl-indazol-5-yl]-4-methyl-pyrimidine-5-carbonyl]amino]cyclobutyl]carbamate (150 mg, 48%) as a solid. LCMS (ESI+) m/z: [M+H]⁺ 511.1. ¹H NMR (400 MHz, CDCl₃) δ ppm 8.75-8.80 (m, 1H) 7.99 (s, 1H) 7.95 (s, 1H) 4.89 (s, 2H) 4.57-4.64 (m, 1H) 4.25-4.37 (m, 4H) 3.36-3.41 (m, 3H) 2.92 (br dd, J=7.09, 1.97 Hz, 1H) 2.75 (s, 3H) 2.66 (s, 3H) 2.43-2.46 (m, 3H) 1.46 (s, 9H).

### Synthesis of Intermediate G288

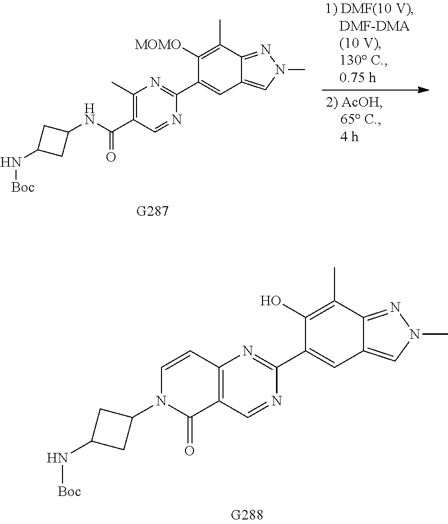

To a solution of tert-butyl N-[3-[[2-[6-(methoxymethoxy)-2,7-dimethyl-indazol-5-yl]-4-methyl-pyrimidine-5-carbonyl]amino]cyclobutyl]carbamate (150 mg, 293 umol, 1 eq) in N,N-dimethylformamide (1.50 mL) was added DMF-DMA (1.50 mL). The reaction mixture was stirred at 130° C. for 45 min, then concentrated under reduced pressure to give a residue. The residue was dissolved in acetic acid (1.50 mL), then stirred at 65° C. for 4 h. The reaction mixture was filtered and the filter cake was washed with methyl tert-butyl ether (3×1.00 mL) to give tert-butyl N-[3-[2-(6-hydroxy-2,7-dimethyl-indazol-5-yl)-5-oxo-pyrido[4,3-d]pyrimidin-6-yl]cyclobutyl]carbamate (90.0 mg, 64%) as a solid. LCMS (ESI+) m/z: [M+H]⁺ 477.0. ¹H NMR (400 MHz, CDCl₃) δ ppm 13.07-13.37 (m, 1H) 9.64 (s, 1H) 9.05 (s, 1H) 7.99 (s, 1H) 7.69 (br d, J=7.39 Hz, 1H) 6.71 (d, J=7.63 Hz, 1H) 5.35-5.47 (m, 1H) 4.97 (br s, 1H) 4.24 (s, 4H) 2.62-2.77 (m, 4H) 2.61 (s, 3H) 1.49 (s, 9H).

### Synthesis of Intermediate G289 and G290

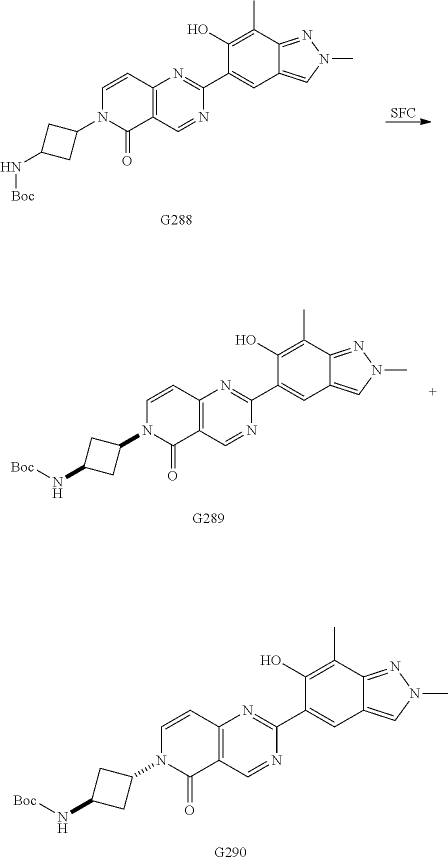 [SMILES: Cc1c(O)c(-c2ncc3c(=O)n(C4CC(NC(=O)OC(C)(C)C)C4)ccc3n2)cc2cn(C)nc12 ; "SFC" ; Cc1c(O)c(-c2ncc3c(=O)n([C@H]4C[C@@H](NC(=O)OC(C)(C)C)C4)ccc3n2)cc2cn(C)nc12 ; Cc1c(O)c(-c2ncc3c(=O)n([C@H]4C[C@H](NC(=O)OC(C)(C)C)C4)ccc3n2)cc2cn(C)nc12]

Tert-butyl N-[3-[2-(6-hydroxy-2,7-dimethyl-indazol-5-yl)-5-oxo-pyrido[4,3-d]pyrimidin-6-yl]cyclobutyl]carbamate (110 mg) was purified by SFC (column: daicel chiralcel od(250 mm*30 mm, 10 um); mobile phase: [0.10% NH₃H₂O EtOH]; B %: 50%-50%, 13 min) to give tert-butyl ((1s,3s)-3-(2-(6-hydroxy-2,7-dimethyl-2H-indazol-5-yl)-5-oxopyrido[4,3-d]pyrimidin-6(5H)-yl)cyclobutyl)carbamate (25.0 mg, 22%) and tert-butyl ((1r,3r)-3-(2-(6-hydroxy-2,7-dimethyl-2H-indazol-5-yl)-5-oxopyrido[4,3-d]pyrimidin-6(5H)-yl)cyclobutyl)carbamate (35.0 mg, 31%) as solids. B289: SFC (Rt=1.730 min). LCMS (ESI+) m/z: [M+H]+: 477.22. ¹H NMR (400 MHz, CDCl₃) δ ppm 13.22 (br s, 1H) 9.64 (s, 1H) 9.05 (s, 1H) 7.99 (s, 1H) 7.69-7.85 (m, 1H) 6.71 (d, J=7.58 Hz, 1H) 4.76-4.94 (m, 2H) 4.24 (s, 3H) 3.91-4.05 (m, 1H) 2.92-3.05 (m, 2H) 2.58 (s, 3H) 2.46 (br dd, J=7.15, 2.75 Hz, 2H) 1.48 (s, 9H). B290: SFC (Rt=1.926 min). LCMS (ESI+) m/z: [M+H]⁺ 477.2. ¹H NMR (400 MHz, CDCl₃) δ ppm 13.20 (s, 1H), 9.64 (s, 1H), 9.05 (s, 1H), 7.99 (s, 1H), 7.69 (br d, J=7.58 Hz, 1H), 6.71 (d, J=7.70 Hz, 1H), 5.33-5.47 (m, 1H), 4.83-5.02 (m, 1H), 4.26-4.34 (m, 1H), 4.24 (s, 3H), 2.61-2.76 (m, 4H), 2.58 (s, 3H), 1.49 (s, 9H).

### Synthesis of Compound 1431

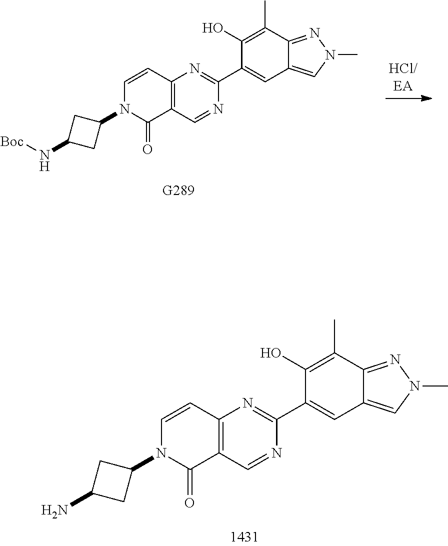 [SMILES: Cc1c(O)c(-c2ncc3c(=O)n([C@H]4C[C@@H](NC(=O)OC(C)(C)C)C4)ccc3n2)cc2cn(C)nc12 ; Cc1c(O)c(-c2ncc3c(=O)n([C@H]4C[C@@H](N)C4)ccc3n2)cc2cn(C)nc12]

To a solution of tert-butyl ((1s,3s)-3-(2-(6-hydroxy-2,7-dimethyl-2H-indazol-5-yl)-5-oxopyrido[4,3-d]pyrimidin-6(5H)-yl)cyclobutyl)carbamate (25.0 mg, 1 eq) in ethyl acetate (250 uL) was added HCl/ethyl acetate (250 uL, 4 M). The reaction mixture was stirred at 25° C. for 1 h, then filtered. The filter cake was washed with dichloromethane (3.00 mL), then dried to give 6-((1s,3s)-3-aminocyclobutyl)-2-(6-hydroxy-2,7-dimethyl-2H-indazol-5-yl)pyrido[4,3-d]pyrimidin-5(6H)-one (20.0 mg, 80%) as a solid. LCMS (ESI+) m/z: [M+H]⁺ 377.2. ¹H NMR (400 MHz, D₂O) δ ppm 8.25 (s, 1H) 7.73 (s, 1H) 7.55 (br d, J=6.72 Hz, 2H) 6.12 (br d, J=7.34 Hz, 1H) 4.47 (br t, J=8.19 Hz, 1H) 3.71-3.80 (m, 4H) 2.92 (br d, J=7.58 Hz, 2H) 2.38-2.46 (m, 2H) 1.58 (s, 3H).

### Example G141: Synthesis of Compound 1432

### Synthesis of Compound 1432

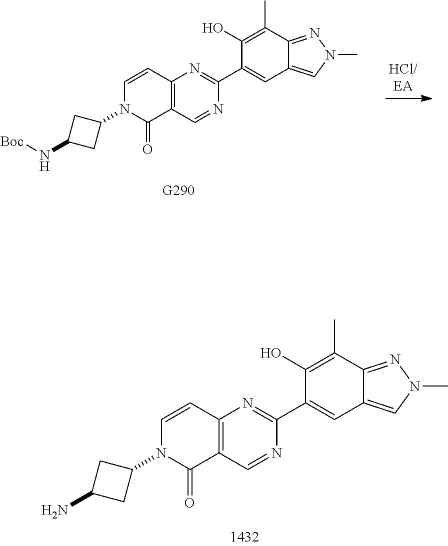 [SMILES: Cc1c(O)c(-c2ncc3c(=O)n([C@H]4C[C@H](NC(=O)OC(C)(C)C)C4)ccc3n2)cc2cn(C)nc12 ; Cc1c(O)c(-c2ncc3c(=O)n([C@H]4C[C@H](N)C4)ccc3n2)cc2cn(C)nc12]

To a solution of tert-butyl ((1r,3r)-3-(2-(6-hydroxy-2,7-dimethyl-2H-indazol-5-yl)-5-oxopyrido[4,3-d]pyrimidin-6(5H)-yl)cyclobutyl)carbamate (35.0 mg, 1 eq) in ethyl acetate (350 uL) was added HCl/ethyl acetate (350 uL, 4 M) at 15° C. The mixture was stirred at 15° C. for 1 h, then filtered. The filter cake was washed with dichloromethane (3×1.00 mL) and dried to give 6-((1r,3r)-3-aminocyclobutyl)-2-(6-hydroxy-2,7-dimethyl-2H-indazol-5-yl)pyrido[4,3-d]pyrimidin-5(6H)-one (30.0 mg, 84%) as a solid. LCMS (ESI+) m/z: [M+H]⁺ 377.2. ¹H NMR (400 MHz, D₂O) δ ppm 8.27 (s, 1H) 7.73 (s, 1H) 7.49-7.61 (m, 2H) 6.05 (d, J=7.46 Hz, 1H) 5.00-5.09 (m, 1H) 3.95-4.03 (m, 1H) 3.78 (s, 3H) 2.67-2.83 (m, 4H) 1.56 (s, 3H).

### Example G142: Synthesis of Compounds 640 and 641

### Synthesis of Intermediate G291

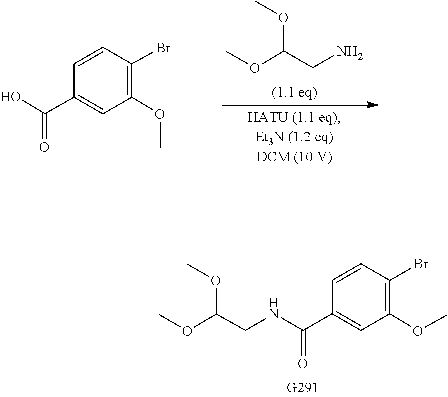

To a solution of 4-bromo-3-methoxy-benzoic acid (10.0 g, 43.3 mmol, 1 eq) in dichloromethane (100 mL) was added O-(7-Azabenzotriazol-1-yl)-N,N,N′,N′-tetramethyl-uronium hexafluorophosphate (18.1 g, 47.6 mmol, 1.1 eq) and triethylamine (5.26 g, 51.9 mmol, 7.23 mL, 1.2 eq). The reaction mixture was stirred at 20° C. for 15 min. To the reaction mixture was added 2,2-dimethoxyethanamine (5.01 g, 47.6 mmol, 5.19 mL, 1.1 eq). The resulting mixture was stirred at 20° C. for 1 h, then washed with water (5×50.0 mL). The organic layer was dried over anhydrous Na₂SO₄, filtered, and the filtrate concentrated to give a residue, which was purified by silica gel column chromatography, eluted with 5-40% of ethyl acetate in petroleum ether to afford 4-bromo-N-(2,2-dimethoxyethyl)-3-methoxy-benzamide (31-2, 11.0 g, 68%) as a solid. LCMS: (ES, m/z): 285.9 [M+H−32]⁺. ¹H NMR (400 MHz, CHLOROFORM-d) δ ppm 7.59 (d, J=8.07 Hz, 1H) 7.44 (d, J=1.83 Hz, 1H) 7.12 (dd, J=8.13, 1.90 Hz, 1H) 6.33 (br s, 1H) 4.50 (t, J=5.20 Hz, 1H) 3.96 (s, 3H) 3.61 (t, J=5.50 Hz, 2H) 3.45 (s, 6H).

### Synthesis of Intermediate G292

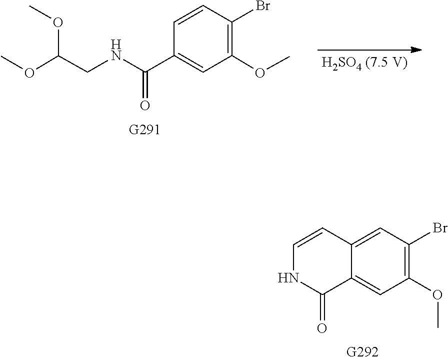

4-bromo-N-(2,2-dimethoxyethyl)-3-methoxy-benzamide (5.00 g, 15.7 mmol, 1 eq) was added to H₂SO₄ (37.5 mL) at 0° C. The reaction mixture was stirred at 70° C. for 3 h, then poured into ice water (300 mL) slowly. A precipitate formed that was collected by filtration. The filter cake was washed with water (3×50.0 mL) and dried to give 6-bromo-7-methoxy-2H-isoquinolin-1-one (31-3, 4.00 g, 87%) as a solid. LCMS: (ES, m/z): 253.9, 255.9[M+H]⁺. ¹H NMR (400 MHz, DMSO-d₆) δ ppm 11.33 (br s, 1H) 8.02 (s, 1H) 7.66 (s, 1H) 7.10 (t, J=6.30 Hz, 1H) 6.50 (d, J=6.97 Hz, 1H) 3.95 (s, 3H).

### Synthesis of Intermediate G293

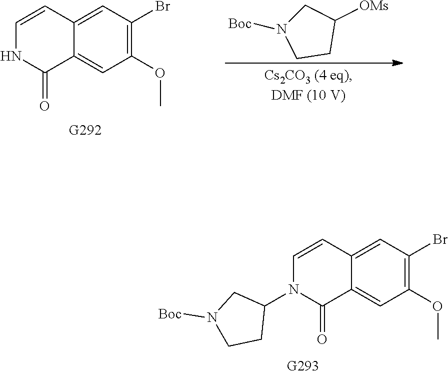 [SMILES: COc1cc2c(=O)[nH]ccc2cc1Br ; CC(C)(C)OC(=O)N1CCC(OS(C)(=O)=O)C1 ; COc1cc2c(=O)n(C3CCN(C(=O)OC(C)(C)C)C3)ccc2cc1Br]

To a solution of 6-bromo-7-methoxy-2H-isoquinolin-1-one (1.00 g, 3.94 mmol, 1 eq) in dimethyl formamide (10.0 mL) was added Cs₂CO₃ (5.13 g, 15.7 mmol, 4 eq) and tert-butyl 3-methylsulfonyloxy pyrrolidine-1-carboxylate (1.25 g, 4.72 mmol, 1.2 eq). The reaction mixture was stirred at 90° C. for 1 h, then poured into water (50.0 mL) and extracted with ethyl acetate (5×20.0 mL). The organic layers were combined, washed with brine (50.0 mL), dried with anhydrous Na₂SO₄, filtered, and the filtrate concentrated under reduced pressure to give a residue, which was purified by silica gel column chromatography, eluted with 10%-60% of ethyl acetate in petroleum ether to give tert-butyl 3-(6-bromo-7-methoxy-1-oxo-2-isoquinolyl)pyrrolidine-1-carboxylate (600 mg, 36%) as a solid. LCMS: (ES, m/z): 367.0 [M−55]⁺. ¹H NMR (400 MHz, DMSO-d₆) δ ppm 8.03 (s, 1H) 7.71 (s, 1H) 7.37 (br s, 1H) 6.64 (d, J=7.58 Hz, 1H) 5.38 (br t, J=6.97 Hz, 1H) 3.96 (s, 3H) 3.70 (br d, J=6.72 Hz, 1H) 3.51 (br dd, J=5.32, 3.12 Hz, 1H) 3.35-3.45 (m, 2H) 2.19-2.30 (m, 2H) 1.40 (s, 9H).

### Synthesis of Intermediate G294

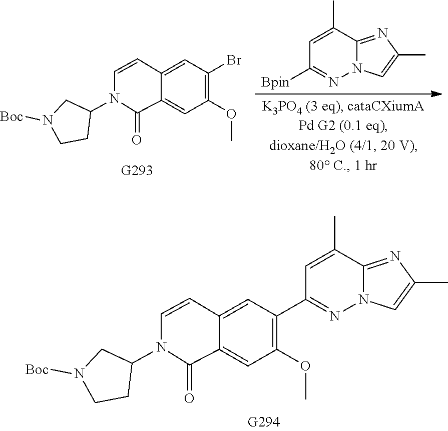

To a mixture of tert-butyl 3-(6-bromo-7-methoxy-1-oxo-2-isoquinolyl)pyrrolidine-1-carboxylate (200 mg, 472 umol, 1 eq) and 2,8-dimethyl-6-(4,4,5,5-tetramethyl-1,3,2-dioxaborolan-2-yl)imidazo[1,2-b]pyridazine (277 mg, 709 umol, 1.5 eq) in a mixture of dioxane (4.00 mL) and water (1.00 mL) was added K₃PO₄ (301 mg, 1.42 mmol, 3 eq) and [2-(2-aminophenyl)phenyl]-chloro-palladium; bis(1-adamantyl)-butyl-phosphane (31.6 mg, 47.3 umol, 0.1 eq) under N₂ atmosphere. The reaction mixture was stirred at 80° C. for 1 h, then cooled to 20° C. The reaction mixture was filtered, the filter cake washed with dichloromethane (5×10.0 mL), and the filtrate concentrated under reduced pressure to give a residue. The residue was purified by silica gel column chromatography, eluted with 30%-100% of ethyl acetate in petroleum ether to give tert-butyl-3-[6-(2,8-dimethylimidazo[1,2-b]pyridazin-6-yl)-7-methoxy-1-oxo-2-isoquinolyl]pyrrolidine-1-carboxylate (31-5, 150 mg, 65%) as a solid. LCMS: (ES, m/z): 490.2 [M+H]⁺.

### Synthesis of Intermediate G295

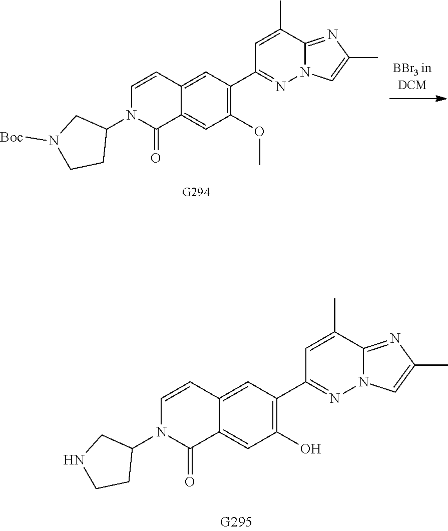 [SMILES: COc1cc2c(=O)n(C3CCN(C(=O)OC(C)(C)C)C3)ccc2cc1-c1cc(C)c2nc(C)cn2n1 ; Cc1cn2nc(-c3cc4ccn(C5CCNC5)c(=O)c4cc3O)cc(C)c2n1]

To a solution of tert-butyl 3-[6-(2,8-dimethylimidazo[1,2-b]pyridazin-6-yl)-7-methoxy-1-oxo-2-isoquinolyl]pyrrolidine-1-carboxylate (150 mg, 307 umol, 1 eq) in dichloromethane (7.50 mL) was added BBr₃ (1 M, 3.0 mL, 9.79 eq) at −20° C. The reaction mixture was stirred at 20° C. for 6 h, then dried under N₂ to give a solid. The solid was triturated with dichloromethane (10.0 mL). The resulting suspension was filtered and the filter cake was concentrated to give 6-(2,8-dimethylimidazo[1,2-b]pyridazin-6-yl)-7-hydroxy-2-pyrrolidin-3-yl-isoquinolin-1-one (120 mg, 76%) as a solid. LCMS: (ES, m/z): 376.2 [M+H]⁺. ¹H NMR (400 MHz, METHANOL-d₄) δ ppm 8.32 (s, 1H) 8.24 (s, 1H) 8.05 (s, 1H) 7.92 (s, 1H) 7.28 (d, J=7.51 Hz, 1H) 6.75 (d, J=7.39 Hz, 1H) 5.06-5.14 (m, 1H) 3.85-3.92 (m, 1H) 3.77-3.82 (m, 1H) 3.66-3.71 (m, 1H) 3.36-3.42 (m, 1H) 2.78 (s, 3H) 2.66-2.73 (m, 4H) 2.48 (td, J=9.63, 4.23 Hz, 1H).

### Synthesis of Compound 640 and 641

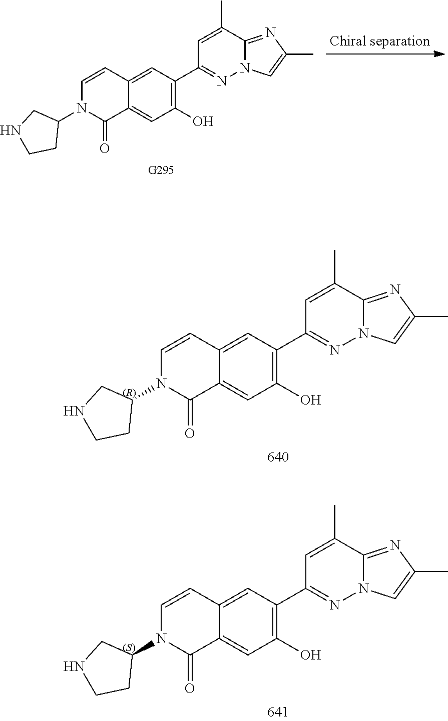

To a solution of 6-(2,8-dimethylimidazo[1,2-b]pyridazin-6-yl)-7-hydroxy-2-pyrrolidin-3-yl-isoquinolin-1-one (120 mg) in methanol (100 mL) was added basic free resin. The resulting mixture was stirred at 20° C. for 1 h, then filtered, and the filtrate concentrated to give a residue (100 mg). The residue was purified by Chiral separation (column: DAICEL CHIRALPAK AD (250 mm×30 mm, 10 um); mobile phase: [0.1% NH₃H₂O ethanol]; B %: 50%-50%, 15 min) to give 6-(2,8-dimethylimidazo[1,2-b]pyridazin-6-yl)-7-hydroxy-2-[(3R)-pyrrolidin-3-yl]isoquinolin-1-one (29.5 mg, 20%) and 6-(2,8-dimethylimidazo[1,2-b]pyridazin-6-yl)-7-hydroxy-2-[(3S)-pyrrolidin-3-yl]isoquinolin-1-one (32.1 mg, 22%) as solids. 640: LCMS: (ES, m/z): 376.0 [M+H]⁺. ¹H NMR (400 MHz, METHANOL-d₄) δ ppm 8.00 (s, 1H) 7.94 (s, 1H) 7.82 (s, 1H) 7.66 (s, 1H) 7.28 (d, J=7.46 Hz, 1H) 6.75 (d, J=7.34 Hz, 1H) 5.27-5.45 (m, 1H) 3.32-3.38 (m, 2H) 2.99-3.15 (m, 2H) 2.66 (s, 3H) 2.50 (s, 3H) 2.33-2.44 (m, 1H) 2.00-2.12 (m, 1H). 641: LCMS: (ES, m/z): 376.1 [M+H]⁺. ¹H NMR (400 MHz, METHANOL-d₄) δ ppm 7.98 (s, 1H) 7.92 (s, 1H) 7.80 (s, 1H) 7.65 (s, 1H) 7.27 (d, J=7.58 Hz, 1H) 6.73 (d, J=7.46 Hz, 1H) 5.30-5.46 (m, 1H) 3.33-3.37 (m, 1H) 3.27 (br s, 1H) 2.98-3.14 (m, 2H) 2.65 (s, 3H) 2.49 (s, 3H) 2.32-2.43 (m, 1H) 2.01-2.12 (m, 1H).

### Example G143: Synthesis of Compounds 1434 and 1441

### Synthesis of Intermediate G296, G297 and G298

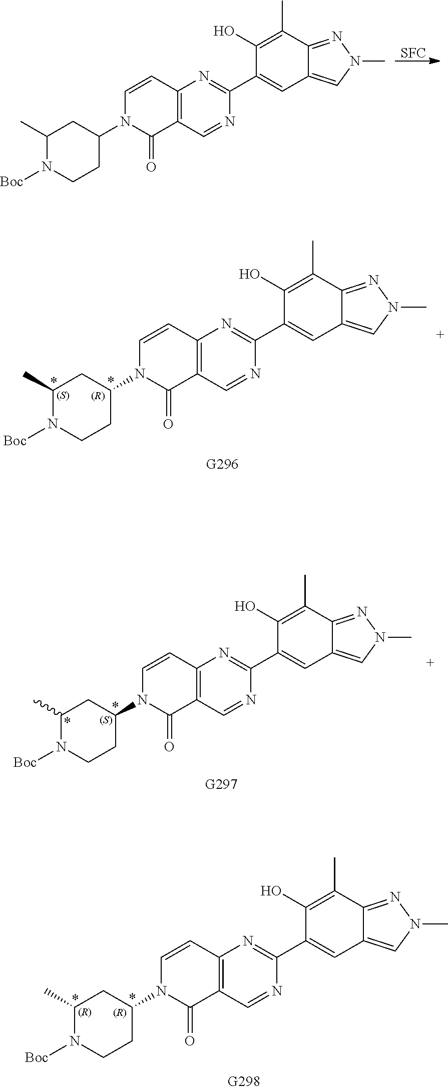

4-[2-(6-hydroxy-2,7-dimethyl-indazol-5-yl)-5-oxo-pyrido[4,3-d]pyrimidin-6-yl]-2-methyl-piperidine-1-carboxylate (200 mg, 396 umol) was purified by SFC (Column: DAICEL CHIRALPAK IE(250 mm*30 mm, 10 um); Mobile phase: A for Heptane and B for EtOH:ACN=4:1; Gradient: B %=50-50%, Flow rate:50 ml/min; Wavelength:220 nm) to give (2S,4R)-tert-butyl 4-(2-(6-hydroxy-2,7-dimethyl-2H-indazol-5-yl)-5-oxopyrido[4,3-d]pyrimidin-6(5H)-yl)-2-methylpiperidine-1-carboxylate (0.8 mg, 1%), (4S)-tert-butyl 4-(2-(6-hydroxy-2,7-dimethyl-2H-indazol-5-yl)-5-oxopyrido[4,3-d]pyrimidin-6(5H)-yl)-2-methylpiperidine-1-carboxylate (44.9 mg, 22%), and (2R,4R)-tert-butyl 4-(2-(6-hydroxy-2,7-dimethyl-2H-indazol-5-yl)-5-oxopyrido[4,3-d]pyrimidin-6(5H)-yl)-2-methylpiperidine-1-carboxylate (29.1 mg, 15%) as solids. B296: LCMS: (ESI, m/z): 505.3 [M+H]⁺. ¹H NMR (400 MHz, METHANOL-d₄) δ ppm 9.67-9.55 (m, 1H) 9.10-9.00 (m, 1H) 8.35-8.25 (m, 1H) 8.06-7.93 (m, 1H) 6.77 (d, J=7.7 Hz, 1H) 5.40-5.26 (m, 1H) 4.84-4.51 (m, 1H) 4.44-4.42 (m, 1H) 4.19 (s, 3H) 3.25-3.12 (m, 2H) 2.44 (s, 3H) 2.17-2.01 (m, 1H) 1.94-1.83 (m, 3H) 1.50 (s, 9H) 1.37-1.34 (m, 3H). B298: LCMS: (ESI, m/z): 505.3 [M+H]⁺. ¹H NMR (400 MHz, METHANOL-d₄) δ ppm 9.51 (s, 1H) 8.97 (s, 1H) 8.25 (s, 1H) 7.97 (d, J=7.83 Hz, 1H) 6.74 (d, J=7.70 Hz, 1H) 4.89-4.99 (m, 1H) 4.18 (s, 3H), 4.00-4.12 (m, 1H), 3.85 (ddd, J=14.00, 7.27, 2.69 Hz, 1H), 3.46 (ddd, J=14.15, 10.18, 6.36 Hz, 1H), 2.41 (s, 3H) 2.24-2.38 (m, 1H), 2.07-2.20 (m, 1H), 1.93-2.03 (m, 1H), 1.72-1.83 (m, 1H), 1.52 (s, 9H), 1.31 (d, J=6.36 Hz, 3H).

### Synthesis of Intermediate G299

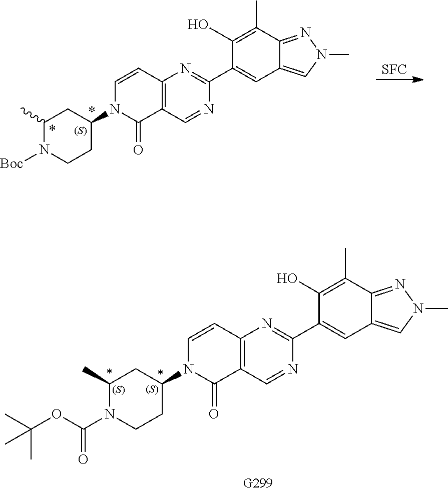

(4S)-tert-butyl 4-(2-(6-hydroxy-2,7-dimethyl-2H-indazol-5-yl)-5-oxopyrido[4,3-d]pyrimidin-6(5H)-yl)-2-methylpiperidine-1-carboxylate (44.9 mg, 89 umol) was purified by SFC (Column: DAICEL CHIRALCEL OD(250 mm*30 mm, 10 um); Mobile phase: A for CO₂ and B for MeOH(0.1% NH₃H₂O); Gradient: B %=50% isocratic elution mode; Flow rate: 70 g/min; Wavelength:220 nm; Column temperature: 35 degrees centigrade; System back pressure: 120 bar) to give (2S,4S)-tert-butyl 4-(2-(6-hydroxy-2,7-dimethyl-2H-indazol-5-yl)-5-oxopyrido[4,3-d]pyrimidin-6(5H)-yl)-2-methylpiperidine-1-carboxylate (18.0 mg, 40%) as a solid. LCMS: (ESI, m/z): 505.3 [M+H]⁺. ¹H NMR (400 MHz, METHANOL-d₄) δ ppm 9.50 (s, 1H) 8.97 (s, 1H) 8.25 (s, 1H) 7.97 (d, J=7.70 Hz, 1H) 6.74 (d, J=7.70 Hz, 1H) 4.89-4.97 (m, 1H) 4.17 (s, 3H) 3.99-4.11 (m, 1H) 3.85 (ddd, J=14.09, 7.37, 2.38 Hz, 1H) 3.46 (ddd, J=14.03, 10.12, 6.30 Hz, 1H) 2.41 (s, 3H) 2.25-2.37 (m, 1H) 2.08-2.18 (m, 1H) 1.95-2.02 (m, 1H) 1.77 (dtd, J=12.99, 6.40, 6.40, 3.18 Hz, 1H), 1.52 (s, 9H) 1.31 (d, J=6.36 Hz, 3H).

### Synthesis of Compound 1434

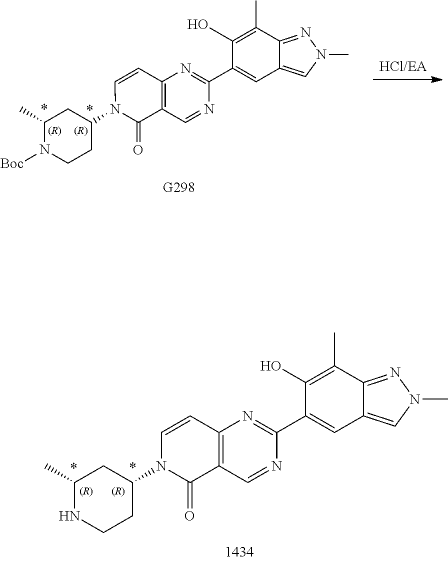

A mixture of (2R,4R)-tert-butyl 4-(2-(6-hydroxy-2,7-dimethyl-2H-indazol-5-yl)-5-oxopyrido[4,3-d]pyrimidin-6(5H)-yl)-2-methylpiperidine-1-carboxylate (20-1P3, 29.1 mg, 57.6 umol, 1 eq) in HCl/ethyl acetate (4.38 mL) was degassed and purged with N₂ 3 times. The resulting mixture was stirred at 20° C. for 4 h under N₂ atmosphere, then concentrated to give 2-(6-hydroxy-2,7-dimethyl-indazol-5-yl)-6-(2-methyl-4-piperidyl)pyrido[4,3-d]pyrimidin-5-one (25.4 mg, 96%) as a solid. LCMS: (ESI, m/z): 405.1 [M+H]⁺. ¹H NMR (400 MHz, METHANOL-d₄) δ ppm 9.65-9.71 (m, 1H) 9.23-9.28 (m, 1H) 8.80-8.84 (m, 1H) 7.94-8.00 (m, 1H) 6.85-6.90 (m, 1H) 5.10 (tt, J=12.05, 4.02 Hz, 1H) 4.32-4.38 (m, 3H) 3.58-3.66 (m, 1H) 3.46-3.57 (m, 1H) 3.33 (br s, 1H) 2.48 (s, 3H) 2.08-2.38 (m, 4H) 1.45 (d, J=6.50 Hz, 3H).

### Synthesis of Compound 1441

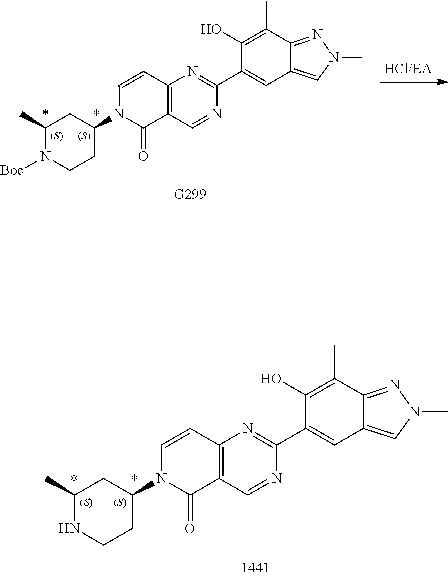

A mixture of (2R,4S)-tert-butyl (2S,4S)-tert-butyl 4-(2-(6-hydroxy-2,7-dimethyl-2H-indazol-5-yl)-5-oxopyrido[4,3-d]pyrimidin-6(5H)-yl)-2-methylpiperidine-1-carboxylate (18.4 mg, 36.47 umol, 1 eq) in HCl/ethyl acetate (4.00 mL) was stirred at 20° C. for 4 h. The reaction mixture was concentrated under reduced pressure to give 2-(6-hydroxy-2,7-dimethyl-indazol-5-yl)-6-(2-methyl-4-piperidyl)pyrido[4,3-d]pyrimidin-5-one (10.16 mg, 41%) as a solid. LCMS: (ESI, m/z): 405.1 [M+H]^{+ 1}H NMR (400 MHz, METHANOL-d₄) δ ppm 9.62-9.70 (m, 1H) 9.22-9.28 (m, 1H) 8.78-8.85 (m, 1H) 7.98 (d, J=7.88 Hz, 1H) 6.84-6.90 (m, 1H) 5.10 (tt, J=12.04, 4.03 Hz, 1H) 4.26-4.40 (m, 3H) 3.59-3.67 (m, 1H) 3.49-3.58 (m, 1H) 3.32-3.36 (m, 1H) 2.47 (s, 3H) 2.09-2.36 (m, 4H) 1.45 (d, J=6.50 Hz, 3H).

### Example G144: Synthesis of Compound 1443

### Synthesis of Intermediate G300 and G301

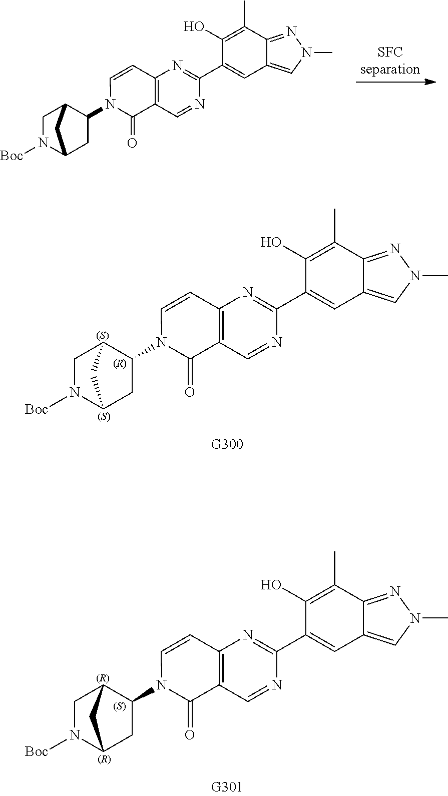

Tert-butyl (4R)-5-[(6R)-2-(6-hydroxy-2,7-dimethyl-indazol-5-yl)-5-oxo-pyrido[4,3-d]pyrimidin-6-yl]-2-azabicyclo[2.2.1]heptane-2-carboxylate (45.0 mg, 89.5 umol, 1 eq) was purified by SFC (column: REGIS(S,S)WHELK-01 (250 mm*25 mm, 10 um); mobile phase: [0.1% NH₃H₂O ETOH]; B %: 70%-70%, 15 min) to give tert-butyl (1S,4S,5R)-5-[2-(6-hydroxy-2,7-dimethyl-indazol-5-yl)-5-oxo-pyrido[4,3-d]pyrimidin-6-yl]-2-azabicyclo[2.2.1]heptane-2-carboxylate (14.0 mg, 31%) and tert-butyl (1R,4R,5S)-5-[2-(6-hydroxy-2,7-dimethyl-indazol-5-yl)-5-oxo-pyrido[4,3-d]pyrimidin-6-yl]-2-azabicyclo[2.2.1]heptane-2-carboxylate (15.0 mg, 33%) as solids. B300: LCMS: (ESI, m/z): 503.3 [M+H]⁺. ¹H NMR (400 MHz, CHLOROFORM-d) δ ppm 13.18 (s, 1H) 9.65 (s, 1H) 9.04 (s, 1H) 7.98 (s, 1H) 7.65 (br d, J=7.70 Hz, 1H) 6.66 (d, J=7.70 Hz, 1H) 4.79-5.00 (m, 1H) 4.29-4.50 (m, 1H) 4.23 (s, 3H) 3.23-3.48 (m, 2H) 2.62-2.85 (m, 2H) 2.57 (s, 3H) 1.67-1.94 (m, 3H) 1.50 (s, 9H). B301: LCMS: (ESI, m/z): 503.3 [M+H]⁺. ¹H NMR (400 MHz, CHLOROFORM-d) δ ppm 13.19 (s, 1H) 9.66 (s, 1H) 9.04 (s, 1H) 7.99 (s, 1H) 7.65 (d, J=7.95 Hz, 1H) 6.67 (d, J=7.70 Hz, 1H) 4.77-5.00 (m, 1H) 4.30-4.53 (m, 1H) 4.23 (s, 3H) 3.23-3.48 (m, 2H) 2.62-2.86 (m, 2H) 2.57 (s, 3H) 1.67-1.95 (m, 3H) 1.50 (s, 9H).

### Synthesis of Compound 1443

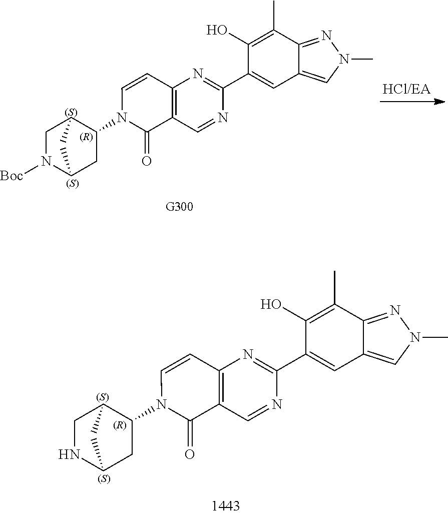

To a solution of tert-butyl (1R,4R,5R)-5-[2-(6-hydroxy-2,7-dimethyl-indazol-5-yl)-5-oxo-pyrido[4,3-d]pyrimidin-6-yl]-2-azabicyclo[2.2.1]heptane-2-carboxylate (10.0 mg, 19.9 umol, 1 eq) in ethyl acetate (100 uL) was added HCl/EtOAc (4 M, 1.00 mL). The reaction mixture was stirred at 15° C. for 12 h, then concentrated under reduced pressure to give a solid. The solid was dissolved in dichloromethane and the resulting mixture was concentrated under reduced pressure to give a residue. The residue was triturated with dichloromethane to form a suspension. The resulting suspension was filtered and the collected solid dried under vacuum to afford 6-[(1R,4R,5R)-2-azabicyclo[2.2.1]heptan-5-yl]-2-(6-hydroxy-2,7-dimethyl-indazol-5-yl)pyrido[4,3-d]pyrimidin-5-one (6.93 mg, 79%) as a solid. LCMS: (ESI, m/z): 403.1 [M+H]⁺. ¹H NMR (400 MHz, METHANOL-d₄) δ ppm 9.65 (s, 1H) 9.24 (s, 1H) 8.78 (s, 1H) 8.14 (d, J=7.89 Hz, 1H) 6.85 (d, J=7.67 Hz, 1H) 4.79-4.83 (m, 1H) 4.33 (s, 3H) 4.26 (br s, 1H) 3.36-3.42 (m, 1H) 3.27 (br s, 1H) 3.17 (br d, J=2.19 Hz, 1H) 2.64-2.71 (m, 1H) 2.47 (s, 3H) 2.19 (br d, J=12.06 Hz, 1H) 2.05-2.12 (m, 1H) 1.98 (br dd, J=11.73, 0.77 Hz, 1H).

### Example G145: Synthesis of Compound 1444

### Synthesis of Compound 1444

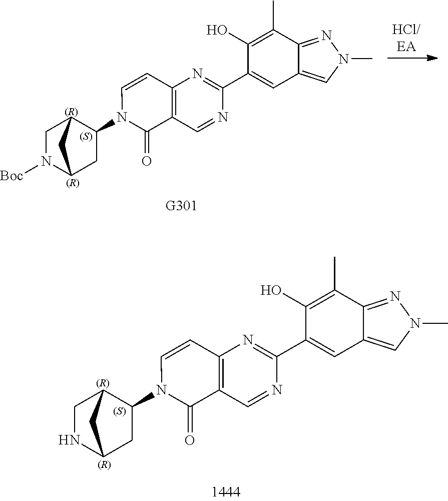

To a solution of tert-butyl (1R,4R,5R)-5-[2-(6-hydroxy-2,7-dimethyl-indazol-5-yl)-5-oxo-pyrido[4,3-d]pyrimidin-6-yl]-2-azabicyclo[2.2.1]heptane-2-carboxylate in ethyl acetate (110 uL) was added HCl/EtOAc (4 M, 1.10 mL). The reaction mixture was stirred at 15° C. for 12 h, then concentrated under reduced pressure to give a solid. The resulting solid was dissolved in dichloromethane and the resulting mixture was concentrated under reduced pressure to give a residue. The residue was triturated with dichloromethane to give a suspension. The resulting suspension was filtered to collect the solid, and the filter cake was dried under vacuum to afford 6-[(1R,4R,5R)-2-azabicyclo[2.2.1]heptan-5-yl]-2-(6-hydroxy-2,7-dimethyl-indazol-5-yl)pyrido[4,3-d]pyrimidin-5-one (7.70 mg, 80%) as a solid. LCMS: (ESI, m/z): 403.1 [M+H]⁺. ¹H NMR (400 MHz, METHANOL-d₄) δ ppm 9.66 (s, 1H) 9.24 (s, 1H) 8.79 (s, 1H) 8.14 (d, J=7.67 Hz, 1H) 6.85 (d, J=7.89 Hz, 1H) 4.81 (br dd, J=7.78, 4.49 Hz, 1H) 4.34 (s, 3H) 4.26 (br s, 1H) 3.36-3.42 (m, 1H) 3.27 (br s, 1H) 3.18 (br d, J=2.19 Hz, 1H) 2.64-2.71 (m, 1H) 2.47 (s, 3H) 2.19 (br d, J=12.06 Hz, 1H) 2.06-2.13 (m, 1H) 1.99 (br s, 1H).

### Example G146: Synthesis of Compound 1445

### Synthesis of Compound 1445

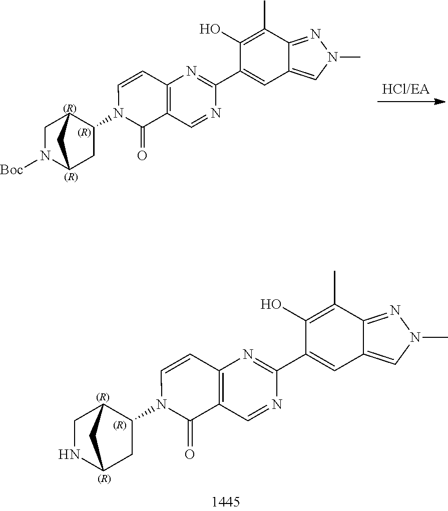

To a solution of tert-butyl (1R,4R,5R)-5-[2-(6-hydroxy-2,7-dimethyl-indazol-5-yl)-5-oxo-pyrido[4,3-d]pyrimidin-6-yl]-2-azabicyclo[2.2.1]heptane-2-carboxylate (19.0 mg, 37.81 umol, 1 eq) in ethyl acetate (190 uL) was added HCl/EtOAc (4 M, 1.90 mL). The reaction mixture was stirred at 15° C. for 12 h, then concentrated under reduced pressure to give a solid. The solid was dissolved in dichloromethane and the resulting mixture was concentrated under reduced pressure to give a residue. The residue was triturated with dichloromethane to form a suspension. The resulting suspension was filtered and the filter cake dried under vacuum to afford 6-[(1R,4R,5R)-2-azabicyclo[2.2.1]heptan-5-yl]-2(6-hydroxy-2,7-dimethyl-indazol-5-yl)pyrido[4,3-d]pyrimidin-5-one (16.3 mg, 98%) as a solid. LCMS: (ESI, m/z): 403.1 [M+H]⁺. ¹H NMR (400 MHz, METHANOL-d₄) δ ppm 9.67 (s, 1H) 9.25 (s, 1H) 8.77 (s, 1H) 8.08 (d, J=7.67 Hz, 1H) 6.88 (d, J=7.67 Hz, 1H) 5.02-5.07 (m, 1H) 4.33 (s, 3H) 4.28 (br s, 1H) 3.37 (s, 1H) 3.28 (br s, 1H) 3.17-3.24 (m, 1H) 2.41-2.52 (m, 5H) 2.19 (br d, J=11.84 Hz, 1H) 1.96-2.02 (m, 1H).

### Example G147: Synthesis of Compound

### Synthesis of Intermediate G302 and G303

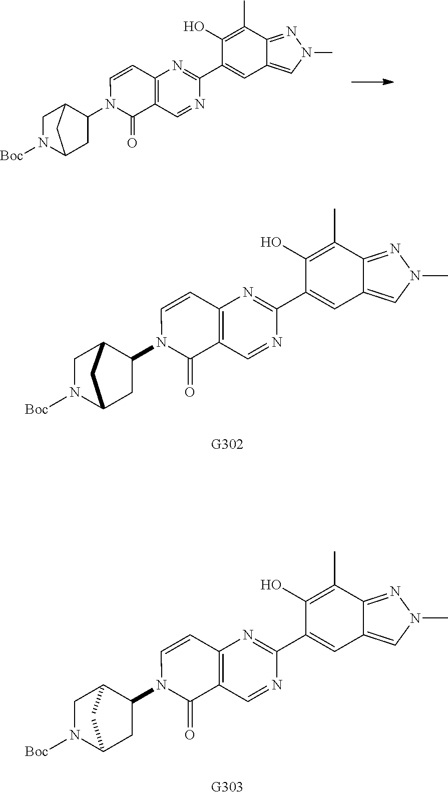

Tert-butyl 5-(2-(6-hydroxy-2,7-dimethyl-2H-indazol-5-yl)-5-oxopyrido[4,3-d]pyrimidin-6(5H)-yl)-2-azabicyclo[2.2.1]heptane-2-carboxylate (150 mg, 98.8 umol, 1 eq) was purified by SFC (column: daicel chiralcel oz 250×25 mm I.D. 10 um; mobile phase: [0.1% NH₃H₂O methanol]; B %: 60%-70%, 30 min) to give tert-butyl (1R,4R,5S)-5-(2-(6-hydroxy-2,7-dimethyl-2H-indazol-5-yl)-5-oxopyrido[4,3-d]pyrimidin-6(5H)-yl)-2-azabicyclo[2.2.1]heptane-2-carboxylate (45.0 mg) and tert-butyl (1S,4S,5S)-5-(2-(6-hydroxy-2,7-dimethyl-2H-indazol-5-yl)-5-oxopyrido[4,3-d]pyrimidin-6(5H)-yl)-2 azabicyclo[2.2.1]heptane-2-carboxylate (70.0 mg) as solids. B302: LCMS: (ESI, m/z): 503.3 [M+H]⁺. ¹H NMR (400 MHz, CHLOROFORM-d) δ ppm 13.18 (br s, 1H) 9.64 (s, 1H) 9.03 (s, 1H) 7.98 (s, 1H) 7.64 (d, J=7.70 Hz, 1H) 6.66 (d, J=7.70 Hz, 1H) 4.72-5.02 (m, 1H) 4.27-4.53 (m, 1H) 4.23 (s, 3H) 3.21-3.49 (m, 2H) 2.60-2.89 (m, 2H) 2.56 (s, 3H) 1.73-1.96 (m, 3H) 1.49 (s, 9H). LCMS: (ESI, m/z): 503.3 [M+H]⁺. B303: LCMS: (ESI, m/z): 503.3 [M+H]⁺. ¹H NMR (400 MHz, CHLOROFORM-d) δ ppm 13.06-13.30 (m, 1H) 9.65 (s, 1H) 9.04 (s, 1H) 7.98 (s, 1H) 7.50 (br d, J=7.21 Hz, 1H) 6.66 (br d, J=7.46 Hz, 1H) 5.51 (br d, J=10.27 Hz, 1H) 4.31-4.53 (m, 1H) 4.23 (s, 3H) 3.06-3.33 (m, 2H) 3.00 (br s, 1H) 2.57 (s, 3H) 1.84-2.27 (m, 4H) 1.52 (br s, 9H).

### Synthesis of Intermediate G304 and G305

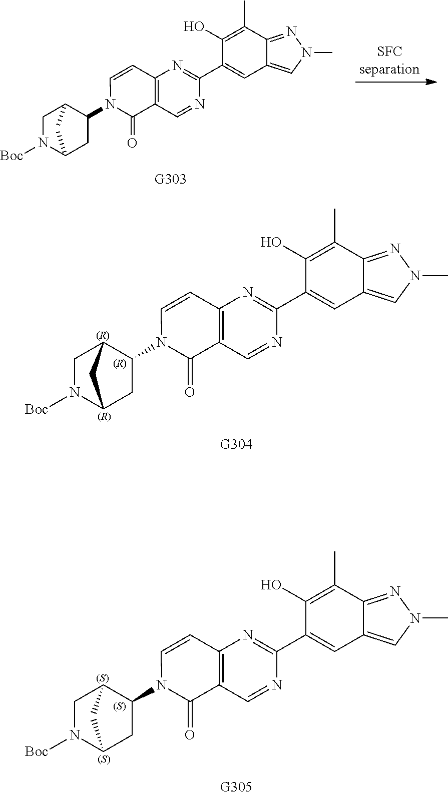

Tert-butyl (4S,5S)-5-[2-(6-hydroxy-2,7-dimethyl-indazol-5-yl)-5-oxo-pyrido[4,3-d]pyrimidin-6-yl]-2-azabicyclo[2.2.1]heptane-2-carboxylate (70.0 mg, 139 umol, 1 eq) was purified by SFC (column: DAICEL CHIRALPAK IG (250 mm*30 mm, 10 um); mobile phase: [Neu-ETOH]; B %: 60%-60%, 12 min) to afford tert-butyl (1R,4R,5R)-5-[2-(6-hydroxy-2,7-dimethyl-indazol-5-yl)-5-oxo-pyrido[4,3-d]pyrimidin-6-yl]-2-azabicyclo[2.2.1]heptane-2-carboxylate (23.0 mg, 33%) and tert-butyl (1S,4S,5S)-5-[2-(6-hydroxy-2,7-dimethyl-indazol-5-yl)-5-oxo-pyrido[4,3-d]pyrimidin-6-yl]-2-azabicyclo[2.2.1]heptane-2-carboxylate (22.0 mg, 31%) as solids. B303: LCMS: (ESI, m/z): 503.3 [M+H]⁺. ¹H NMR (400 MHz, CHLOROFORM-d) δ ppm 13.06-13.34 (m, 1H) 9.66 (s, 1H) 9.05 (s, 1H) 7.99 (s, 1H) 7.41-7.55 (m, 1H) 6.67 (br d, J=6.97 Hz, 1H) 5.47-5.61 (m, 1H) 4.32-4.49 (m, 1H) 4.24 (s, 3H) 3.07-3.33 (m, 2H) 3.01 (br s, 1H) 2.58 (s, 3H) 1.86-2.28 (m, 4H) 1.52 (br s, 9H). B304: LCMS: (ESI, m/z) [M+H]⁺ 503.3. ¹H NMR (400 MHz, CHLOROFORM-d) δ ppm 13.06-13.34 (m, 1H) 9.66 (s, 1H) 9.05 (s, 1H) 7.99 (s, 1H) 7.41-7.55 (m, 1H) 6.67 (br d, J=6.97 Hz, 1H) 5.47-5.61 (m, 1H) 4.32-4.49 (m, 1H) 4.24 (s, 3H) 3.07-3.33 (m, 2H) 3.01 (br s, 1H) 2.58 (s, 3H) 2.14-2.23 (m, 2H) 1.86-1.95 (m, 2H) 1.52 (br s, 9H).

### Synthesis of Compound 1446

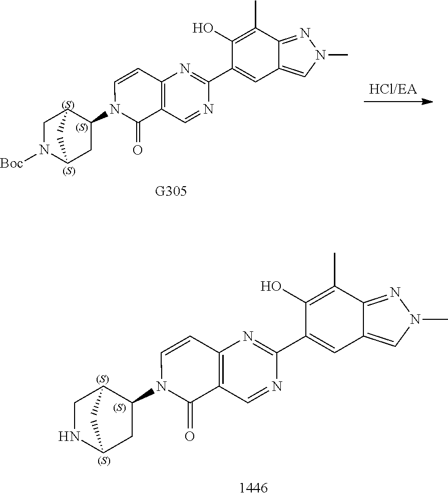

To a solution of tert-butyl (1R,4R,5R)-5-[2-(6-hydroxy-2,7-dimethyl-indazol-5-yl)-5-oxo-pyrido[4,3-d]pyrimidin-6-yl]-2-azabicyclo[2.2.1]heptane-2-carboxylate (18.0 mg, 35.8 umol, 1 eq) in ethyl acetate (180 uL) was added HCl/EtOAc (4 M, 1.80 mL). The resulting mixture was stirred at 15° C. for 12 h, then concentrated under reduced pressure to give a solid. The solid was dissolved in dichloromethane and the resulting mixture was concentrated under reduced pressure to give a residue. The residue was triturated with dichloromethane to give a suspension. The suspension was filtered to collect the solid, and filter cake was dried under vacuum to afford 6-[(1R,4R,5R)-2-azabicyclo[2.2.1]heptan-5-yl]-2-(6-hydroxy-2,7-dimethyl-indazol-5-yl)pyrido[4,3-d]pyrimidin-5-one (14.2 mg, 90%) as a solid. LCMS: (ESI, m/z): 403.1 [M+H]⁺. ¹H NMR (400 MHz, METHANOL-d₄) δ ppm 9.67 (s, 1H) 9.25 (s, 1H) 8.77 (s, 1H) 8.08 (d, J=7.67 Hz, 1H) 6.88 (d, J=7.67 Hz, 1H) 5.02-5.07 (m, 1H) 4.33 (s, 3H) 4.28 (br s, 1H) 3.37 (s, 1H) 3.28 (br s, 1H) 3.17-3.24 (m, 1H) 2.41-2.52 (m, 5H) 2.19 (br d, J=11.84 Hz, 1H) 1.96-2.02 (m, 1H).

### Example G148: Synthesis of Compound 643

### Synthesis of Intermediate G306

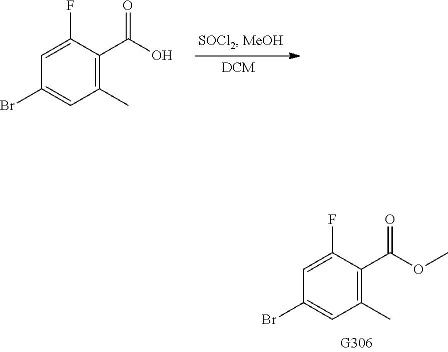

A mixture of 4-bromo-2-fluoro-6-methyl-benzoyl chloride (15.0 g, 59.6 mmol, 1 eq) and SOCl₂ (35.4 g, 298 mmol, 21.6 mL, 5 eq) in DCM (150 mL) under N₂ atmosphere was stirred at 45° C. for 4 h, then poured into methanol (150 mL) at 0° C. The resulting mixture was stirred at 25° C. for 4 h, then quenched with water (100 mL) at 0° C. and diluted with ethyl acetate (100 mL). The aqueous layer was extracted with ethyl acetate (3×100 mL). The organic layers were combined, washed with water (100 mL), dried over MgSO₄, filtered, and the filtrate concentrated under reduced pressure to give a residue. The residue was triturated with tert-butyl methyl ether (10.0 mL) at 25° C. for 30 min to give methyl 4-bromo-2-fluoro-6-methyl-benzoate (10.0 g, 54%) as a solid. ¹H NMR (CHLOROFORM-d, 400 MHz) δ 7.1-7.2 (m, 2H), 3.94 (s, 3H), 2.39 (s, 3H).

### Synthesis of Intermediate G307

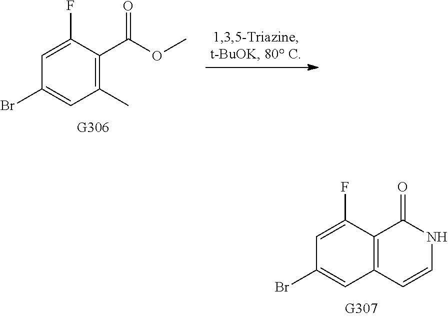

To a solution of methyl 4-bromo-2-fluoro-6-methyl-benzoate (10.0 g, 40.4 mmol, 1.00 eq) in dimethyl sulfoxide (100 mL) was added 1,3,5-triazine (3.94 g, 48.5 mmol, 1.2 eq) and t-BuOK (5.90 g, 52.6 mmol, 1.3 eq). The reaction mixture was stirred at 80° C. for 2 h, then quenched with water (100 mL) at 0° C. and diluted with ethyl acetate (100 mL). The aqueous layer was extracted with ethyl acetate (3×100 mL). The organic layers were combined, washed with water (100 mL), dried over MgSO₄, filtered, and the filtrate concentrated under reduced pressure to give a residue. The residue was triturated with methanol (10.0 mL) at 25° C. for 30 min to form a suspension. The resulting suspension was filtered, and the filter cake dried to afford 6-bromo-8-fluoro-2H-isoquinolin-1-one (4.50 g, 41%) as a solid. LCMS: (ESI, m/z): 241.9, 243.9 [M+H]^{+ 1}H NMR (DMSO-d₆, 400 MHz) δ ppm 11.36 (br s, 1H) 7.77 (s, 1H) 7.48 (dd, 1H, J=1.8, 11.1 Hz) 7.24 (t, 1H, J=6.4 Hz) 6.51 (dd, 1H, J=2.0, 7.1 Hz) ¹⁹F NMR (DMSO-d₆, 376 MHz) δ ppm−108.474 (s, 1F).

### Synthesis of Intermediate G308

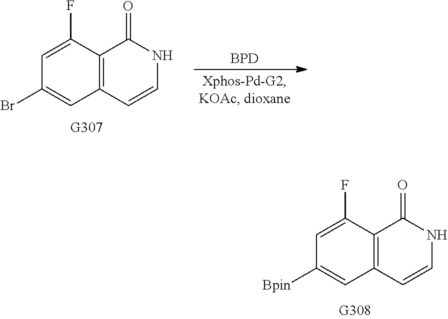

To a solution of 6-bromo-8-fluoro-2H-isoquinolin-1-one (4.50 g, 18.6 mmol, 1.00 eq) in 1,4-dioxane (45.0 mL) was added chloro(2-dicyclohexylphosphino-2′,4′,6′-triisopropyl-1,1′-biphenyl)[2-(2′-amino-1,1′-biphenyl)]palladium(II)(1.46 g, 1.86 mmol, 0.10 eq) and potassium acetate (3.65 g, 37.1 mmol, 2.00 eq), bis(pinacolato)diboron (14.1 g, 55.7 mmol, 3.00 eq). The reaction mixture was stirred at 100° C. for 3 h, then quenched with water (100 mL) at 0° C. and diluted with ethyl acetate (100 mL). The aqueous layer was extracted with ethyl acetate (100 mL). The organic layers were combined, washed with water (100 mL), dried over MgSO₄, filtered, and the filtrate concentrated under reduced pressure to give 8-fluoro-6-(4,4,5,5-tetramethyl-1,3,2-dioxaborolan-2-yl)-2H-isoquinolin-1-one (2.00 g, 33%) as a solid. LCMS: (ESI, m/z): 290.1 [M+H]⁺.

### Synthesis of Intermediate G309

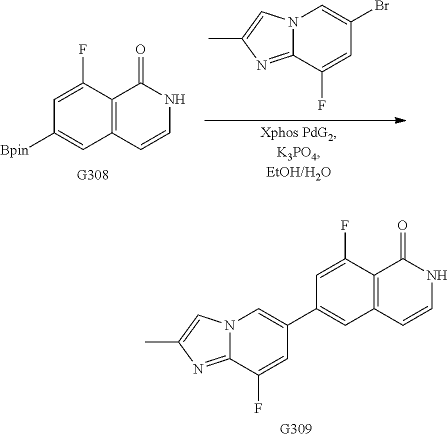

To a mixture of 8-fluoro-6-(4,4,5,5-tetramethyl-1,3,2-dioxaborolan-2-yl)-2H-isoquinolin-1-one (2.00 g, 6.92 mmol, 1.00 eq) and 6-bromo-8-fluoro-2-methyl-imidazo[1,2-a]pyridine (1.43 g, 6.23 mmol, 0.90 eq) in ethanol (20.0 mL) and water (5.00 mL) was added Xphos-Pd-G2 (544 mg, 692 umol, 0.10 eq) and K₃PO₄ (2.94 g, 13.8 mmol, 2.00 eq) under N₂ atmosphere. The resulting mixture was stirred at 80° C. for 12 h, then quenched with water (100 mL) at 0° C. and extracted with ethyl acetate (3×100 mL). The organic layers were combined, washed with water (100 mL), dried over MgSO₄, filtered, and the filtrate concentrated under reduced pressure to give a residue. The residue was re-crystallized from methanol (10.0 mL) at 25° C. to give 8-fluoro-6-(8-fluoro-2-methyl-imidazo[1,2-a]pyridin-6-yl)-2H-isoquinolin-1-one (2.00 g, 84%) as a solid. LCMS: (ESI, m/z): 312.1 [M+H]⁺. ¹H NMR (DMSO-d₆, 400 MHz) δ ppm 8.98 (d, 1H, J=1.3 Hz), 7.85 (dd, 2H, J=2.0, 13.9 Hz), 7.6-7.7 (m, 2H), 7.2-7.3 (m, 1H), 6.56 (br d, 1H, J=7.5 Hz), 2.39 (s, 3H) ¹⁹F NMR (DMSO-d₆, 376 MHz) δ ppm−110.597 (s, 1F), −132.019 (s, 1F).

### Synthesis of Intermediate G310

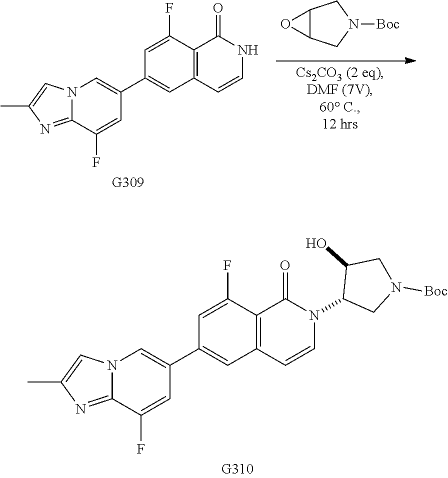

To a mixture of tert-butyl 6-oxa-3-azabicyclo[3.1.0]hexane-3-carboxylate (50.0 mg, 270 umol, 8.40 eq) and 8-fluoro-6-(8-fluoro-2-methyl-imidazo[1,2- a]pyridin-6-yl)-2H-isoquinolin-1-one (10.0 mg, 32.1 umol, 1.00 eq) in N,N-dimethyl formamide (500 uL) was added Cs₂CO₃ (20.9 mg, 64.2 umol, 2.00 eq). The reaction mixture was stirred at 60° C. for 12 h, then quenched with water (10.0 mL) at 25° C. and extracted with ethyl acetate (3×10 mL). The organic layers were combined, washed with brine (10 mL), dried over MgSO₄, filtered, and the filtrate concentrated under reduced pressure to give a residue. The residue was purified by prep-TLC (petroleum ether/ethyl acetate=1/2, Rf=0.25) to give tert-butyl 3-[8-fluoro-6-(8-fluoro-2-methyl-imidazo[1,2-a]pyridin-6-yl)-1-oxo-2-isoquinolyl]-4-hydroxy-pyrrolidine-1-carboxylate (50.0 mg, 28%) as a solid. ¹H NMR (DMSO-d₆, 400 MHz) δ 9.00 (d, 1H, J=1.4 Hz) 7.86 (dd, 2H, J=2.0, 18.0 Hz) 7.6-7.7 (m, 2H) 7.47 (br t, 1H, J=7.3 Hz) 6.70 (dd, 1H, J=1.8, 7.6 Hz) 5.60 (d, 1H, J=5.0 Hz) 5.07 (q, 1H, J=7.2 Hz) 4.4-4.6 (m, 1H) 3.7-3.8 (m, 1H) 3.63 (dd, 1H, J=6.6, 11.0 Hz) 3.4-3.4 (m, 1H) 3.1-3.2 (m, 1H) 2.39 (s, 3H) 1.43 (br d, 9H, J=5.5 Hz).

### Synthesis of Compound 643

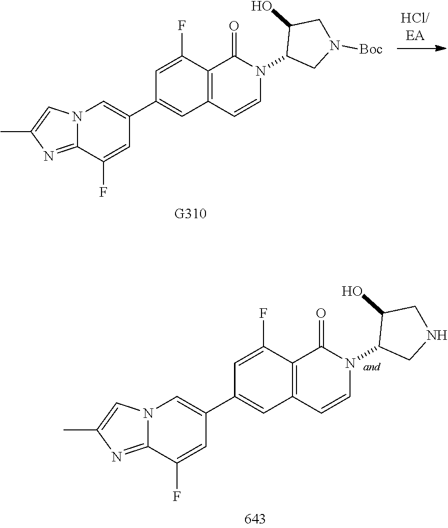

To a solution of tert-butyl 3-[8-fluoro-6-(8-fluoro-2-methyl-imidazo[1,2-a]pyridin-6-yl)-1-oxo-2-isoquinolyl]-4-hydroxy-pyrrolidine-1-carboxylate (50.0 mg, 100 umol, 1.00 eq) in ethyl acetate (1.00 mL) was added HCl/ethyl acetate (4.0 M, 1.50 mL) under N₂ atmosphere. The reaction mixture was stirred at 25° C. for 6 h, then concentrated under reduced pressure to give a residue. The residue was re-crystallized from methanol (10.0 mL) at 25° C. to give 8-fluoro-6-(8-fluoro-2-methyl-imidazo[1,2-a]pyridin-6-yl)-2-(4-hydroxypyrrolidin-3-yl)isoquinolin-1-one (35.2 mg, 79%) as a solid. LCMS: (ESI, m/z): 397.1 [M+H]^{+ 1}H NMR (METHANOL-d₄, 400 MHz) δ 9.12 (d, 1H, J=1.2 Hz), 8.27 (dd, 1H, J=1.2, 11.1 Hz), 8.14 (d, 1H, J=1.1 Hz), 7.91 (d, 1H, J=1.5 Hz), 7.66 (dd, 1H, J=1.7, 12.7 Hz), 7.50 (d, 1H, J=7.5 Hz), 6.84 (dd, 1H, J=2.0, 7.6 Hz), 4.90 (br s, 1H), 4.7-4.7 (m, 1H), 3.8-4.0 (m, 3H), 3.27 (d, 1H, J=5.3 Hz), 2.62 (d, 3H, J=0.9 Hz).

### Example G149: Synthesis of Compound 474

### Synthesis of Intermediate G311

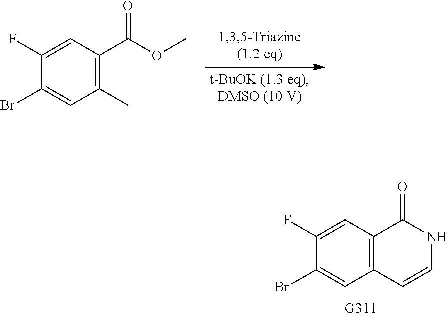

To a solution of ethyl 4-bromo-5-fluoro-2-methyl-benzoate (500 mg, 1.92 mmol, 1 eq) in dimethylsulfoxide (5.00 mL) was added 1,3,5-Triazine (186 mg, 2.30 mmol, 1.2 eq) and potassium tert-butoxide (279 mg, 2.49 mmol, 1.3 eq). The reaction mixture was stirred at 80° C. for 5 h, then poured into water (200 mL) and extracted with ethyl acetate (3×50.0 mL). Nine additional reaction mixtures were set up in parallel. The organic layers were combined, washed with brine (200 mL), dried over anhydrous sodium sulfate, filtered, and the filtrate concentrated under reduced pressure to give a residue, which was purified by silica gel chromatography (petroleum ether/ethyl acetate=1/1) to give a residue. The residue was triturated with methyl tert-butyl ether (10.0 mL) to form a suspension. The resulting suspension was filtered and the filter cake dried to give 6-bromo-7-fluoro-2H-isoquinolin-1-one (900 mg, 22%) as a solid. LCMS: (ES, m/z): 241.9 [M+H]⁺. ¹H NMR (400 MHz, DMSO-d₆) δ ppm 11.48 (br s, 1H) 8.16 (d, J=6.60 Hz, 1H) 7.94 (d, J=9.29 Hz, 1H) 7.16-7.29 (m, 1H) 6.56 (d, J=7.09 Hz, 1H). ¹⁹F NMR (376 MHz, DMSO-d₆) δ ppm−110.020 (s, 1F).

### Synthesis of Intermediate G312

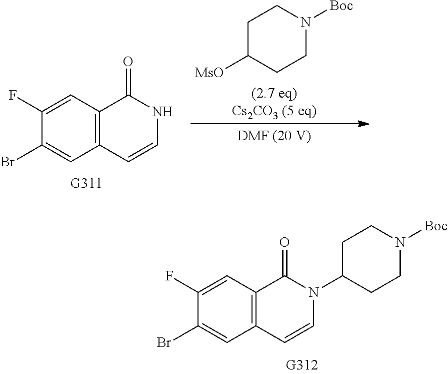 [SMILES: O=c1[nH]ccc2cc(Br)c(F)cc12 ; CC(C)(C)OC(=O)N1CCC(OS(C)(=O)=O)CC1 ; CC(C)(C)OC(=O)N1CCC(n2ccc3cc(Br)c(F)cc3c2=O)CC1]

To a solution of 6-bromo-7-fluoro-2H-isoquinolin-1-one (950 mg, 3.92 mmol, 1 eq) in dimethyl formamide (19.0 mL) was added Cs₂CO₃ (6.39 g, 19.6 mmol, 5 eq) and tert-butyl 4-methylsulfonyloxypiperidine-1-carboxylate (2.96 g, 10.6 mmol, 2.7 eq). The reaction mixture was stirred at 80° C. for 1 h, then cooled to 20° C. and poured into water (100 mL). The resulting mixture was extracted with dichloromethane (4×10 mL). The organic layers were combined, dried with anhydrous Na₂SO₄, filtered, and the filtrate concentrated under vacuum to give a residue, which was purified by column chromatography on silica gel (13% of ethyl acetate in petroleum ether) to give tert-butyl 4-(6-bromo-7-fluoro-1-oxo-2-isoquinolyl)piperidine-1-carboxylate (600 mg, 36%) as an oil. LCMS: (ES, m/z): 369.0 [M−tBu+H]⁺. ¹H NMR (400 MHz, CHLOROFORM-d) δ ppm 8.13 (d, J=9.05 Hz, 1H) 7.76 (d, J=6.24 Hz, 1H) 7.09 (br d, J=7.58 Hz, 1H) 6.46 (br d, J=7.58 Hz, 1H) 5.14 (br t, J=12.35 Hz, 1H) 4.32 (br d, J=1.22 Hz, 2H) 2.78-3.01 (m, 2H) 1.90 (br d, J=11.74 Hz, 2H) 1.66-1.81 (m, 2H) 1.49 (s, 9H). ¹⁹F NMR (376 MHz, CHLOROFORM-d) δ ppm−107.786 (s, 1F).

### Synthesis of Intermediate G313

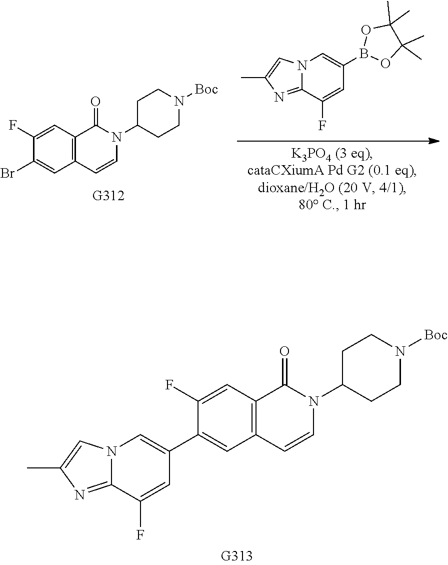

To a mixture of tert-butyl 4-(6-bromo-7-fluoro-1-oxo-2-isoquinolyl)piperidine-1-carboxylate (100 mg, 235 umol, 1 eq) and 8-fluoro-2-methyl-6-(4,4,5,5-tetramethyl-1,3,2-dioxaborolan-2-yl)imidazo[1,2-a]pyridine (71.4 mg, 259 umol, 1.1 eq) in a mixture of dioxane (1.60 mL) and water (0.4 mL) was added tripotassium; phosphate (150 mg, 705 umol, 3 eq) and [2-(2-aminophenyl)phenyl]-chloro-palladium; bis(1-adamantyl)-butyl-phosphane (15.7 mg, 23.5 umol, 0.1 eq) under N₂ atmosphere. The resulting mixture was stirred at 80° C. for 1 h. Two additional reaction mixtures were set up in parallel and the three reaction mixtures were combined. The combined reaction mixture was poured into water (10.0 mL) and extracted with dichloromethane (4×8.00 mL). The organic layers were combined, washed with saturated brine (20 mL), dried over anhydrous sodium sulfate, filtered, and the filtrate concentrated to give a residue, which was purified by silica gel column chromatography (petroleum ether/ethyl acetate=1:1) to give tert-butyl 4-[7-fluoro-6-(8-fluoro-2-methyl-imidazo[1,2-a]pyridin-6-yl)-1-oxo-2-isoquinolyl]piperidine-1-carboxylate (0017-4, 150 mg, 43%) as a solid. LCMS: (ES, m/z): 495.2 [M+H]⁺. ¹H NMR (400 MHz, CHLOROFORM-d) δ ppm 8.17-8.27 (m, 2H) 7.61 (d, J=6.97 Hz, 1H) 7.50 (s, 1H) 7.14 (br d, J=7.46 Hz, 2H) 6.59 (d, J=7.58 Hz, 1H) 5.13-5.26 (m, 1H) 4.27-4.44 (m, 2H) 2.88-3.02 (m, 2H) 2.54 (s, 3H) 1.93 (br dd, J=12.41, 2.75 Hz, 2H) 1.73-1.84 (m, 2H) 1.50 (s, 9H).

### Synthesis of Compound 474

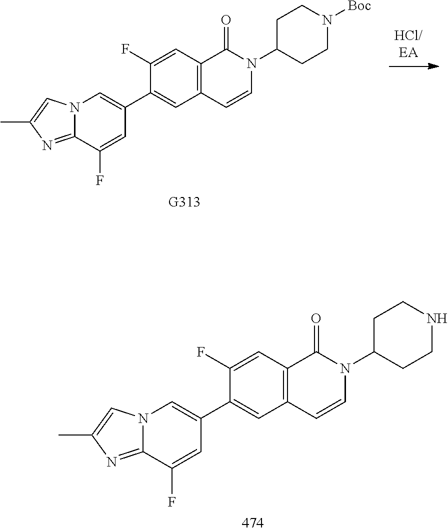 [SMILES: Cc1cn2cc(-c3cc4ccn(C5CCN(C(=O)OC(C)(C)C)CC5)c(=O)c4cc3F)cc(F)c2n1 ; Cc1cn2cc(-c3cc4ccn(C5CCNCC5)c(=O)c4cc3F)cc(F)c2n1]

A mixture of tert-butyl 4-[7-fluoro-6-(8-fluoro-2-methyl-imidazo[1,2-a]pyridin-6-yl)-1-oxo-2-isoquinolyl]piperidine-1-carboxylate (140 mg, 283 umol, 1 eq) in HCl (g)/ethyl acetate (4 M, 20.0 mL) was stirred at 20° C. for 2 h. A solid formed that was collected by filtration. The solid was triturated with dichloromethane (3.00 mL) to form a suspension. The resulting suspension was filtered and the filter cake dried to give 7-fluoro-6-(8-fluoro-2-methyl-imidazo[1,2-a]pyridin-6-yl)-2-(4-piperidyl)isoquinolin-1-one (120 mg, 98%) as a solid. LCMS: (ES, m/z): 395.2 [M+H]⁺. ¹H NMR (400 MHz, METHANOL-d₄) δ ppm 9.01 (s, 1H) 8.18 (d, J=11.00 Hz, 3H) 8.02 (d, J=7.21 Hz, 1H) 7.47 (d, J=7.58 Hz, 1H) 6.88 (d, J=7.58 Hz, 1H) 5.08-5.18 (m, 1H) 3.62 (br d, J=12.96 Hz, 2H) 3.23-3.30 (m, 2H) 2.63 (s, 3H) 2.35 (qd, J=13.08, 4.03 Hz, 2H) 2.17 (br d, J=13.82 Hz, 2H)¹⁹F NMR (376 MHz, METHANOL-d₄) δ ppm−119.159 (s, 1F)-134.020 (s, 1F).

### Synthesis of Intermediate G314

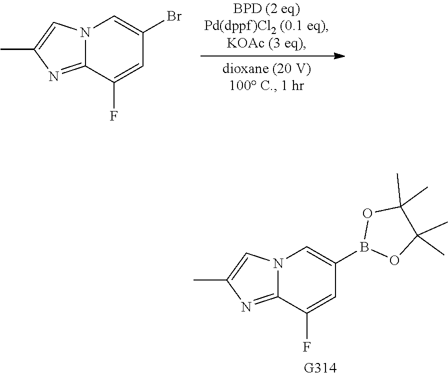

To a solution of 6-bromo-8-fluoro-2-methyl-imidazo[1,2-a]pyridine (1.00 g, 4.37 mmol, 1 eq) in dioxane (20.0 mL) was added potassium acetate (1.29 g, 13.1 mmol, 3 eq), cyclopentyl(diphenyl)phosphane; dichloropalladium; iron (319 mg, 437 umol, 0.1 eq), and 4,4,4′,4′,5,5,5′,5′-octamethyl-2,2′-bi(1,3,2-dioxaborolane)(2.22 g, 8.73 mmol, 2 eq) under N₂ atmosphere. The reaction mixture was stirred at 100° C. for 1 h, then cooled to 20° C., filtered and the filter cake washed with dichloromethane (3×10.0 mL). The filtrate was concentrated under reduced pressure to give 8-fluoro-2-methyl-6-(4,4,5,5-tetramethyl-1,3,2-dioxaborolan-2-yl)imidazo[1,2-a]pyridine (1.20 g, 85%) as a solid. LCMS: (ES, m/z): 194.1 [M−82+H]⁺.

### Example G150: Synthesis of Compound 1449 and 1450

### Synthesis of Intermediate G315

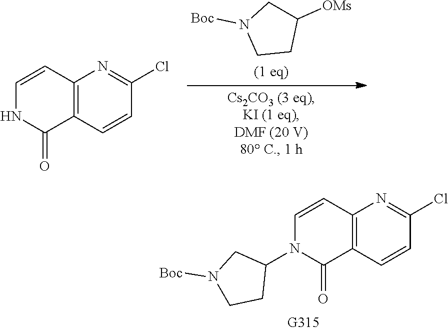 [SMILES: CC(C)(C)OC(=O)N1CCC(OS(C)(=O)=O)C1 ; O=c1[nH]ccc2nc(Cl)ccc12 ; CC(C)(C)OC(=O)N1CCC(n2ccc3nc(Cl)ccc3c2=O)C1]

A mixture of 2-chloro-6H-1,6-naphthyridin-5-one (6.00 g, 33.2 mmol, 1 eq), tert-butyl 3-methylsulfonyloxy pyrrolidine-1-carboxylate (8.82 g, 33.2 mmol, 1 eq), Cs₂CO₃ (32.5 g, 99.7 mmol, 3 eq), and KI (5.52 g, 33.2 mmol, 1 eq) in N,N-dimethylformamide (120 mL) was degassed and purged with N₂ 3 times. The reaction mixture was stirred at 80° C. for 1 h under N₂ atmosphere, then cooled to 25° C., quenched with water (500 mL) and extracted with dichloromethane (6×100 mL). The organic layers were combined, dried over Na₂SO₄, filtered, and the filtrate concentrated under reduced pressure to give a residue. The residue was purified by column chromatography (SiO₂, 23% of ethyl acetate in petroleum ether) to give tert-butyl 3-(2-chloro-5-oxo-1,6-naphthyridin-6-yl)pyrrolidine-1-carboxylate (3.00 g, 13%) as a solid. LCMS: (ES, m/z): 350.1, 352.1 [M+H]⁺. ¹HNMR (400 MHz, CHLOROFORM-d) δ ppm 8.61 (d, J=8.46 Hz, 1H) 7.40 (d, J=8.34 Hz, 1H) 7.35 (br d, J=7.75 Hz, 1H) 6.75 (d, J=7.87 Hz, 1H) 5.53-5.61 (m, 1H) 3.40-3.55 (m, 4H) 2.26 (br d, J=15.14 Hz, 2H) 1.49 (s, 9H).

### Synthesis of Intermediate G316

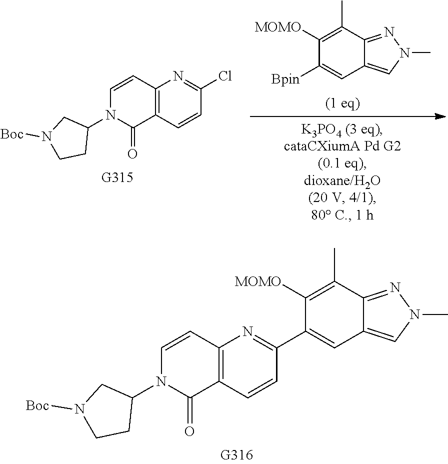

A mixture of 6-(methoxymethoxy)-2,7-dimethyl-5-(4,4,5,5-tetramethyl-1,3,2-dioxaborolan-2-yl)indazole (759 mg, 2.29 mmol, 1 eq), tert-butyl 3-(2-chloro-5-oxo-1,6-naphthyridin-6-yl)pyrrolidine-1-carboxylate (800 mg, 2.29 mmol, 1 eq), [2-(2-aminophenyl)phenyl]-chloro-palladium; bis(1-adamantyl)-butyl-phosphane (153 mg, 22 umol, 0.1 eq), K₃PO₄ (1.46 g, 6.86 mmol, 3 eq) in dioxane (12.8 mL) and H₂O (3.20 mL) was degassed and purged with N₂ 3 times. The reaction mixture was stirred at 80° C. for 1 h under N₂ atmosphere, then cooled to 25° C., quenched with water (20.0 mL), and extracted with ethyl acetate (6×10.0 mL). The organic layers were combined, dried over Na₂SO₄, filtered, and the filtrate concentrated under reduced pressure to give a residue. The residue was purified by column chromatography (SiO₂, 83% of ethyl acetate in petroleum ether) to give tert-butyl 3-[2-[6-(methoxymethoxy)-2,7-dimethyl-indazol-5-yl]-5-oxo-1,6-naphthyridin-6-yl]pyrrolidine-1-carboxylate (160 mg, 13%) as a solid. LCMS: (ES, m/z): 520.3 [M+H]⁺.

### Synthesis of Intermediate G317

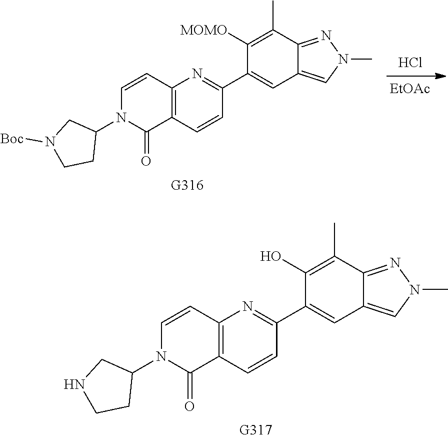

To a solution of tert-butyl 3-[2-[6-(methoxymethoxy)-2,7-dimethyl-indazol-5-yl]-5-oxo-1,6-naphthyridin-6-yl]pyrrolidine-1-carboxylate (160 mg, 308 umol, 1 eq) in ethyl acetate (1.60 mL) was added HCl/ethyl acetate (4.00 M, 1.60 mL). The reaction mixture was stirred at 25° C. for 2 h, then filtered and the filter cake was dried to give a residue. The residue was triturated with dichloromethane (5.00 mL) to form a suspension. The suspension was filtered to collect the solid and the filter cake dried to give 2-(6-hydroxy-2,7-dimethyl-indazol-5-yl)-6-pyrrolidin-3-yl-1,6-naphthyridin-5-one (140 mg, 94%) as a solid. LCMS: (ES, m/z): 376.2 [M+H]⁺.

### Synthesis of Compound 1449 and 1450

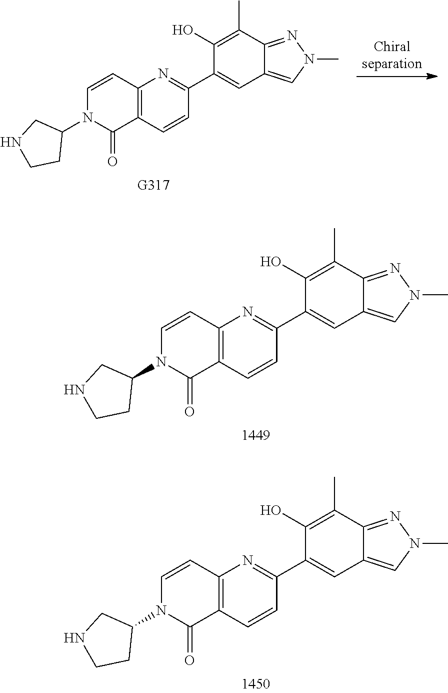 [SMILES: Cc1c(O)c(-c2ccc3c(=O)n(C4CCNC4)ccc3n2)cc2cn(C)nc12 ; "Chiralseparation" ; Cc1c(O)c(-c2ccc3c(=O)n([C@H]4CCNC4)ccc3n2)cc2cn(C)nc12 ; Cc1c(O)c(-c2ccc3c(=O)n([C@@H]4CCNC4)ccc3n2)cc2cn(C)nc12]

2-(6-hydroxy-2,7-dimethyl-indazol-5-yl)-6-pyrrolidin-3-yl-1,6-naphthyridin-5-one (EVAL-0175-0022, 280 mg) was purified by SFC (column: DAICEL CHIRALCEL OJ(250 mm×30 mm, 10 um); mobile phase: [0.1% NH₃H₂O ethanol]; B %: 60%-60%, 20 min) to give 2-(6-hydroxy-2,7-dimethyl-indazol-5-yl)-6-[(3S)-pyrrolidin-3-yl]-1,6-naphthyridin-5-one (76.3 mg, 27%) and 2-(6-hydroxy-2,7-dimethyl-indazol-5-yl)-6-[(3R)-pyrrolidin-3-yl]-1,6-naphthyridin-5-one (68.4 mg, 24%) as solids. 1449: SFC (Rt=2.845 min). LCMS (ESI, m/z): [M+H]⁺ 376.2. ¹H NMR (400 MHz, DMSO-d₆) δ ppm 14.00 (s, 1H) 8.62 (d, J=8.70 Hz, 1H) 8.54 (s, 1H) 8.32-8.43 (m, 2H) 7.93 (d, J=7.75 Hz, 1H) 6.85 (d, J=7.63 Hz, 1H) 5.35 (dt, J=8.20, 3.95 Hz, 1H) 4.14 (s, 3H) 3.08-3.21 (m, 2H) 2.82-2.96 (m, 2H) 2.37 (s, 3H) 2.27-2.33 (m, 1H) 1.81 (br dd, J=13.23, 5.72 Hz, 1H). 1450: SFC (Rt=3.548 min). LCMS (ESI, m/z) m/z: [M+H]⁺ 376.2. ¹H NMR (400 MHz, DMSO-d₆) δ ppm 14.00 (s, 1H) 8.54-8.62 (m, 2H) 8.32-8.43 (m, 2H) 7.93 (s, 1H) 6.85 (d, J=7.63 Hz, 1H) 5.35 (dt, J=8.20, 3.95 Hz, 1H) 4.14 (s, 3H) 3.08-3.21 (m, 2H) 2.82-2.96 (m, 2H) 2.37 (s, 3H) 2.27-2.33 (m, 1H) 1.78 (br dd, J=13.23, 5.72 Hz, 1H).

### Example G151: Synthesis of Compound 1451 and 1452

### Synthesis of Intermediate G318

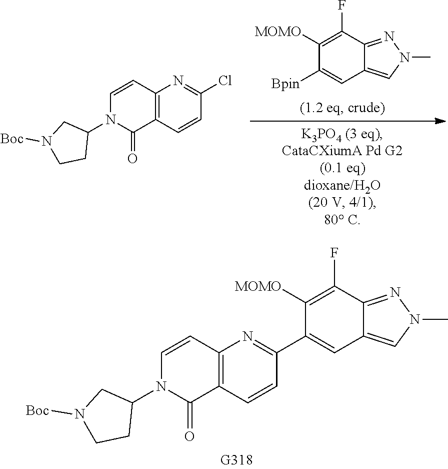

A mixture of 7-fluoro-6-(methoxymethoxy)-2-methyl-5-(4,4,5,5-tetramethyl-1,3,2-dioxaborolan-2-yl)indazole (A23, 824 mg, 1.72 mmol, 70% purity, 1.2 eq), tert-butyl 3-(2-chloro-5-oxo-1,6-naphthyridin-6-yl)pyrrolidine-1-carboxylate (22-4, 500 mg, 1.43 mmol, 1 eq), K₃PO₄ (910 mg, 4.29 mmol, 3 eq), and [2-(2-aminophenyl)phenyl]-chloro-palladium; bis(1-adamantyl)-butyl-phosphane (95.6 mg, 143 umol, 0.1 eq) in dioxane (8.00 mL) and water (2.00 mL) was degassed and purged with N₂ 3 times. The reaction mixture was stirred at 80° C. for 1 h under N₂ atmosphere, then cooled to 25° C., quenched with water (20.0 mL), and extracted with ethyl acetate (6×10.0 mL). The organic layers were combined, dried over Na₂SO₄, filtered, and the filtrate concentrated under reduced pressure to give a residue. The residue was purified by column chromatography (SiO₂, 86% of ethyl acetate in petroleum ether) to give tert-butyl 3-[2-[7-fluoro-6-(methoxymethoxy)-2-methyl-indazol-5-yl]-5-oxo-1,6-naphthyridin-6-yl]pyrrolidine-1-carboxylate (120 mg, 16%) as a solid. LCMS: (ES, m/z): 524.3 [M+H]⁺.

### Synthesis of Intermediate G319 and G320

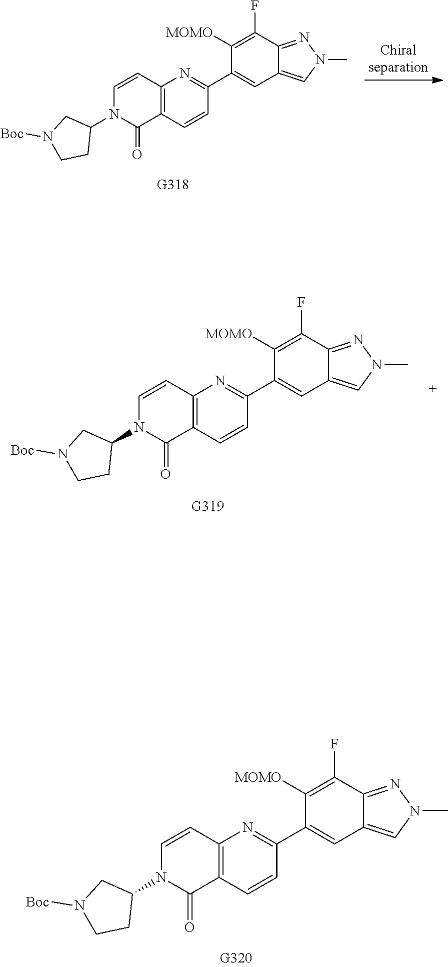

Tert-butyl 3-[2-[7-fluoro-6-(methoxymethoxy)-2-methyl-indazol-5-yl]-5-oxo-1,6-naphthyridin-6-yl]pyrrolidine-1-carboxylate (23-5, 330 mg) was purified by SFC (column: Phenomenex-Cellulose-2 (250 mm×30 mm, 10 um); mobile phase: [0.1%₀NH₃H₂O ethanol]; B %: 50%-50%, 15 min) to give tert-butyl (3S)-3-[2-[7-fluoro-6-(methoxymethoxy)-2-methyl-indazol-5-yl]-5-oxo-1,6-naphthyridin-6-yl]pyrrolidine-1-carboxylate (90.0 mg, 27%) and tert-butyl (3R)-3-[2-[7-fluoro-6-(methoxymethoxy)-2-methyl-indazol-5-yl]-5-oxo-1,6-naphthyridin-6-yl]pyrolidine-1-carboxylate (80.0 mg, 24%) as solids. B319: SFC (Rt=1.413 min). LCMS (ESI, m/z) m/z: [M+H]⁺ 524.3. ¹H NMR (400 MHz, DMSO-d₆) δ ppm 8.55-8.60 (m, 2H) 7.93 (s, 1H) 7.72 (d, J=7.82 Hz, 1H) 7.70 (br d, J=7.95 Hz, 1H) 6.81 (d, J=7.82 Hz, 1H) 5.39 (t, J=6.66 Hz, 1H) 4.96 (s, 2H) 4.21 (s, 3H) 3.72-3.75 (m, 1H) 3.50-3.53 (m, 1H) 3.40-3.42 (m, 2H) 3.16 (s, 3H) 2.23-2.29 (m, 2H) 1.42 (s, 9H). ¹⁹F NMR (376 MHz, methanol-d₄) δ ppm−149.688 (s, 1F). B320: SFC (Rt=1.880 min). LCMS (ESI, m/z) m/z: [M+H]⁺ 524.3. ¹H NMR (400 MHz, DMSO-d₆) δ ppm 8.54-8.64 (m, 2H) 7.94 (s, 1H) 7.87 (d, J=8.46 Hz, 1H) 7.68-7.76 (m, 1H) 6.81 (d, J=7.75 Hz, 1H) 5.39 (br t, J=6.97 Hz, 1H) 4.96 (s, 2H) 4.18-4.24 (m, 3H) 3.67-3.80 (m, 1H) 3.49-3.57 (m, 1H) 3.39-3.45 (m, 2H) 3.16 (s, 3H) 2.21-2.32 (m, 2H) 1.42 (br s, 9H). ¹⁹F NMR (400 MHz, methanol-d₄) δ ppm−149.683 (s, 1F).

### Synthesis of Compound 1451

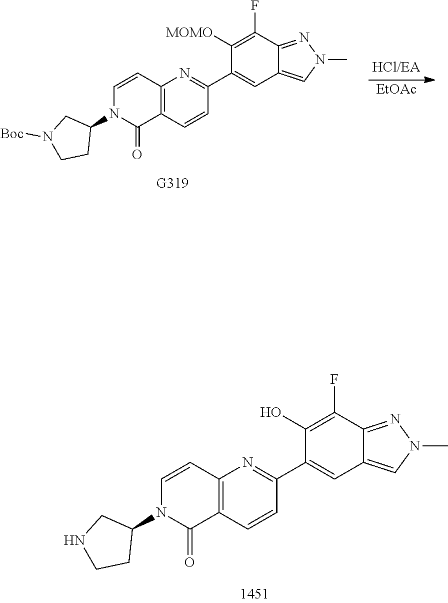

To a mixture of tert-butyl (3 S)-3-[2-[7-fluoro-6-(methoxymethoxy)-2-methyl-indazol-5-yl]-5-oxo-1,6-naphthyridin-6-yl]pyrrolidine-1-carboxylate (23-5P1, 90.0 mg, 172 umol, 1 eq) in ethyl acetate (1.00 mL) was added HCl/ethyl acetate (4.00 M, 1.00 mL). The reaction mixture was stirred at 25° C. for 2 h, then filtered and the filter cake was dried to give a residue. The residue was triturated with dichloromethane (5.00 mL) to give a suspension. The resulting suspension was filtered and the filter cake was dried to give 2-(7-fluoro-6-hydroxy-2-methyl-indazol-5-yl)-6-[(3S)-pyrrolidin-3-yl]-1,6-naphthyridin-5-one (66.9 mg, 87%) as a solid. LCMS (ESI+) m/z: [M+H]⁺ 380.1. ¹H NMR (400 MHz, DMSO-d₆) δ ppm 8.65 (d, J=8.68 Hz, 1H) 8.49-8.61 (m, 2H) 8.39 (d, J=8.93 Hz, 1H) 7.93 (dd, J=7.70, 1.47 Hz, 1H) 6.95 (d, J=7.70 Hz, 1H) 5.27-5.42 (m, 1H) 4.17 (s, 3H) 3.61 (dt, J=11.77, 4.33 Hz, 2H) 3.51-3.56 (m, 1H) 3.21-3.33 (m, 1H) 2.19-2.32 (m, 2H). ¹⁹F NMR (376 MHz, DMSO-d₆) δ ppm−157.723 (s, 1F).

### Synthesis of Compound 1452

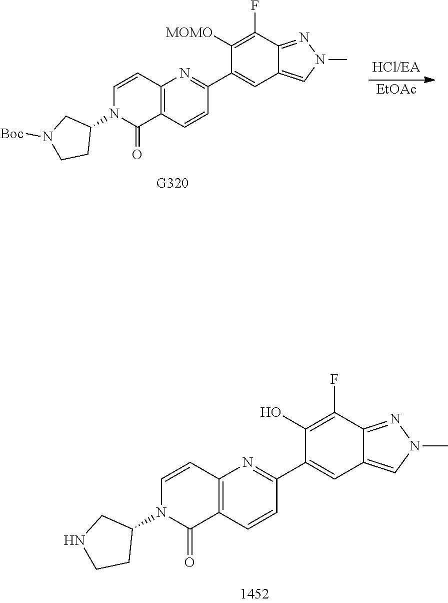

To a mixture of tert-butyl (3R)-3-[2-[7-fluoro-6-(methoxymethoxy)-2-methyl-indazol-5-yl]-5-oxo-1,6-naphthyridin-6-yl]pyrrolidine-1-carboxylate (80.0 mg, 153 umol, 1 eq) in ethyl acetate (1.00 mL) was added HCl/ethyl acetate (4.00 M, 1.00 mL). The reaction mixture was stirred at 25° C. for 2 h, then filtered and the filter cake was dried to give a residue. The residue was triturated with dichloromethane (5.00 mL) to give a suspension. The suspension was filtered and the filter cake was dried to give 2-(7-fluoro-6-hydroxy-2-methyl-indazol-5-yl)-6-[(3R)-pyrrolidin-3-yl]-1,6-naphthyridin-5-one (45.7 mg, 83%) as a solid. LCMS (ESI+) m/z: [M+H]⁺ 380.2. ¹H NMR (400 MHz, DMSO-d₆) δ ppm 8.65 (d, J=8.80 Hz, 1H) 8.58 (d, J=2.57 Hz, 1H) 8.54 (s, 1H) 8.39 (d, J=8.93 Hz, 1H) 7.92 (d, J=7.82 Hz, 1H) 6.95 (d, J=7.83 Hz, 1H) 5.24-5.39 (m, 1H) 4.17 (s, 3H) 3.57-3.65 (m, 2H) 3.50-3.56 (m, 1H) 3.23-3.33 (m, 1H) 2.17-2.32 (m, 2H). ¹⁹F NMR (376 MHz, DMSO-d₆) δ ppm−157.721 (s, 1F).

### Example G152: Synthesis of Compound 1457 and 1458

### Synthesis of Intermediate G324

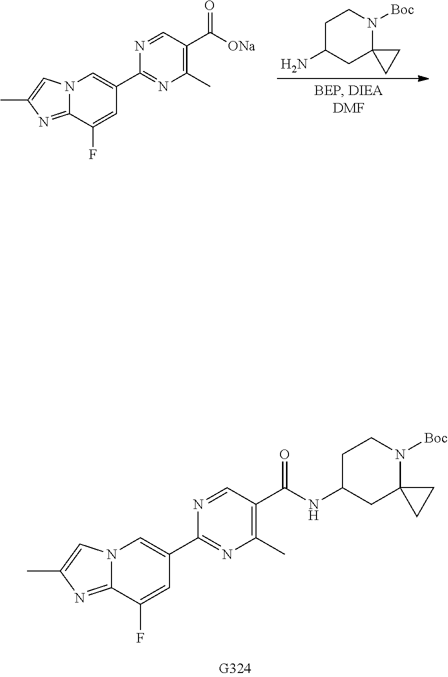

A mixture of sodium 2-(8-fluoro-2-methylimidazo[1,2-a]pyridin-6-yl)-4-methyl-pyrimidine-5-carboxylate (300 mg, 970 umol, 1.00 eq), tert-butyl 7-amino-4-azaspiro[2.5]octane-4-carboxylate (263 mg, 1.16 mmol, 1.20 eq), 2-bromo-1-ethyl-pyridin-1-ium; tetrafluoroborate (318 mg, 1.16 mmol, 1.20 eq), and N-ethyl-N-isopropyl-propan-2-amine (313 mg, 2.43 mmol, 423 uL, 2.50 eq) in N,N-dimethylformamide (15.0 mL) was stirred at 30° C. for 2 h, then filtered. The filter cake was washed with methyl tert-butyl ether (3×3.00 mL) and dried under vacuum to give tert-butyl 7-[[2-(8-fluoro-2-methyl-imidazo[1,2-a]pyridin-6-yl)-4-methyl-pyrimidine-5-carbonyl]amino]-4-azaspiro[2.5]octane-4-carboxylate (300 mg, 58%) as a solid. LCMS: (ESI, m/z): 495.1 [M+H]⁺. ¹HNMR (400 MHz, CDCl₃) δ ppm 8.98 (d, J=1.19 Hz, 1H), 8.47 (s, 1H), 7.82 (d, J=11.44 Hz, 1H), 7.49 (d, J=2.38 Hz, 1H), 6.63 (br d, J=8.11 Hz, 1H), 4.41 (td, J=7.69, 4.05 Hz, 1H), 4.09 (br d, J=14.31 Hz, 1H), 3.07 (br t, J=13.11 Hz, 1H), 2.66 (s, 3H), 2.51 (s, 3H), 2.06-2.18 (m, 1H), 1.88-2.04 (m, 1H), 1.52 (br d, J=3.93 Hz, 2H), 1.48 (s, 9H), 1.24-1.36 (m, 1H), 0.86-0.97 (m, 1H), 0.66 (br t, J=8.11 Hz, 2H).

### Synthesis of Intermediate B325

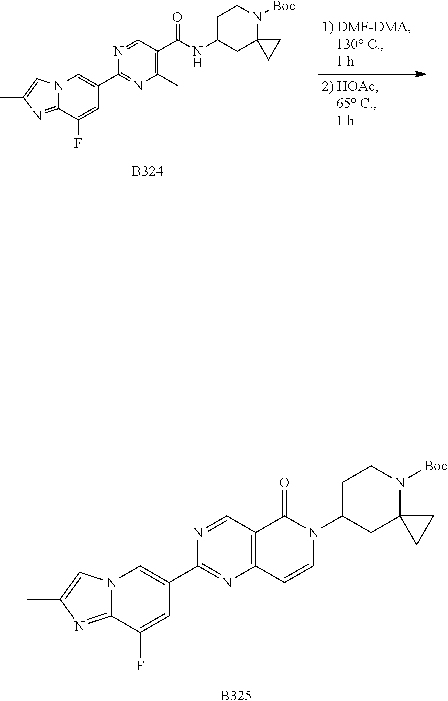 [SMILES: Cc1cn2cc(-c3ncc(C(=O)NC4CCN(C(=O)OC(C)(C)C)C5(CC5)C4)c(C)n3)cc(F)c2n1 ; "B324" ; Cc1cn2cc(-c3ncc4c(=O)n(C5CCN(C(=O)OC(C)(C)C)C6(CC6)C5)ccc4n3)cc(F)c2n1 ; "B325"]

To a solution of tert-butyl 7-[[2-(8-fluoro-2-methyl-imidazo[1,2-a]pyridin-6-yl)-4-methyl-pyrimidine-5-carbonyl]amino]-4-azaspiro[2.5]octane-4-carboxylate (53-4, 300 mg, 606 umol, 1.00 eq) in N,N-dimethylformamide (3.00 mL) was added N,N-dimethylformamide dimethylacetal (2.69 g, 22.5 mmol, 3.00 mL, 37.2 eq). The reaction mixture was stirred at 130° C. for 1 h, then concentrated under reduced pressure to give a residue. The residue was dissolved in acetic acid (3.00 mL) and the reaction mixture was stirred at 65° C. for 1 h, then filtered. The filter cake was washed with methyl tert-butyl ether (3×1.00 mL) and dried under vacuum to give tert-butyl 7-[2-(8-fluoro-2-methyl-imidazo[1,2-a]pyridin-6-yl)-5-oxo-pyrido[4,3-d]pyrimidin-6-yl]-4-azaspiro[2.5]octane-4-carboxylate (200 mg, 70%) as a solid. LCMS: (ESI, m/z): 505.3 [M+H]⁺. ¹HNMR (400 MHz, CDCl₃) δ ppm 9.64 (s, 1H), 9.23 (d, J=1.19 Hz, 1H), 8.03 (dd, J=11.56, 1.19 Hz, 1H), 7.49-7.58 (m, 2H), 6.74 (d, J=7.75 Hz, 1H), 5.31-5.43 (m, 1H), 4.24 (br d, J=13.47 Hz, 1H), 3.10 (br t, J=11.74 Hz, 1H), 2.52 (s, 3H), 1.96 (br dd, J=12.46, 2.09 Hz, 1H), 1.76 (br dd, J=12.40, 3.81 Hz, 1H), 1.52 (s, 9H), 1.35-1.43 (m, 1H), 1.26 (br dd, J=11.74, 3.40 Hz, 1H), 0.78-1.03 (m, 2H), 0.64-0.73 (m, 2H).

### Synthesis of Intermediate B326 and B327

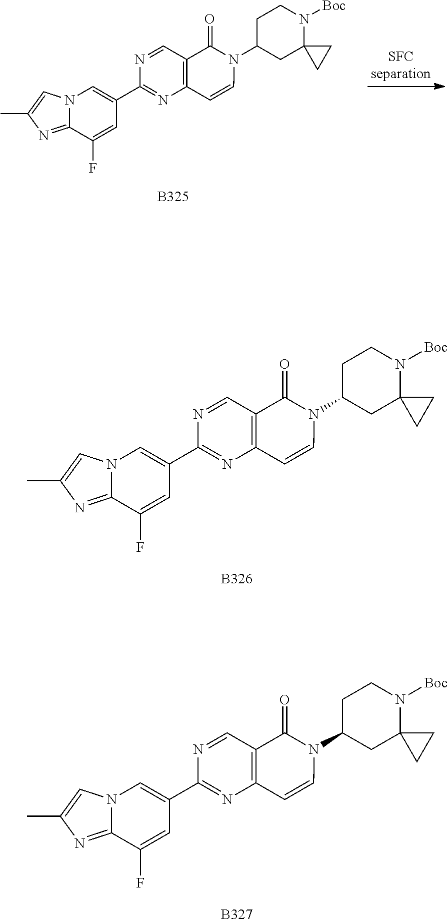 [SMILES: Cc1cn2cc(-c3ncc4c(=O)n(C5CCN(C(=O)OC(C)(C)C)C6(CC6)C5)ccc4n3)cc(F)c2n1 ; "B325" ; Cc1cn2cc(-c3ncc4c(=O)n([C@@H]5CCN(C(=O)OC(C)(C)C)C6(CC6)C5)ccc4n3)cc(F)c2n1 ; "B326" ; Cc1cn2cc(-c3ncc4c(=O)n([C@H]5CCN(C(=O)OC(C)(C)C)C6(CC6)C5)ccc4n3)cc(F)c2n1 ; "B327"]

Tert-butyl 7-[2-(8-fluoro-2-methyl-imidazo[1,2-a]pyridin-6-yl)-5-oxo-pyrido[4,3-d]pyrimidin-6-yl]-4-azaspiro[2.5]octane-4-carboxylate (53-5, 200 mg) was purified by SFC (column: DAICEL CHIRALPAK IG (250 mm×30 mm, 10 um); mobile phase: [0.1% NH3H₂O ETOH]; B %: 50%-50%, 22 min) to give tert-butyl (7R)-7-[2-(8-fluoro-2-methyl-imidazo[1,2-a]pyridin-6-yl)-5-oxo-pyrido[4,3-d]pyrimidin-6-yl]-4-azaspiro[2.5]octane-4-carboxylate (70.0 mg) and tert-butyl (S)-7-(2-(8-fluoro-2-methylimidazo[1,2-a]pyridin-6-yl)-5-oxopyrido[4,3-d]pyrimidin-6(5H)-yl)-4-azaspiro[2.5]octane-4-carboxylate (70.0 mg) as solids. B326: LCMS: (ESI, m/z): 505.0 [M+H]⁺. ¹H NMR (400 MHz, CDCl₃) δ ppm 9.65 (s, 1H), 9.24 (s, 1H), 8.06 (br d, J=11.44 Hz, 1H), 7.47-7.59 (m, 2H), 6.74 (d, J=7.75 Hz, 1H), 5.30-5.44 (m, 1H), 4.18-4.31 (m, 1H), 3.04-3.16 (m, 1H), 2.54 (s, 3H), 2.25 (br t, J=11.80 Hz, 1H), 1.69-1.85 (m, 1H), 1.52 (s, 9H), 1.35-1.43 (m, 1H), 1.26 (br dd, J=11.74, 4.59 Hz, 2H), 0.95 (br d, J=8.11 Hz, 1H), 0.69 (t, J=8.40 Hz, 2H). B327: LCMS: (ESI, m/z): 505.0 [M+H]⁺. ¹HNMR (400 MHz, CDCl₃) δ ppm 9.65 (s, 1H), 9.24 (d, J=0.95 Hz, 1H), 8.07 (br d, J=11.44 Hz, 1H), 7.47-7.60 (m, 2H), 6.74 (d, J=7.75 Hz, 1H), 5.29-5.45 (m, 1H), 4.17-4.31 (m, 1H), 3.03-3.18 (m, 1H), 2.54 (s, 3H), 2.17-2.31 (m, 1H), 1.70-1.84 (m, 1H), 1.52 (s, 9H), 1.40 (br d, J=7.27 Hz, 1H), 1.24-1.29 (m, 2H), 0.95 (br d, J=7.87 Hz, 1H), 0.69 (t, J=8.29 Hz, 2H).

### Synthesis of Compound 1457

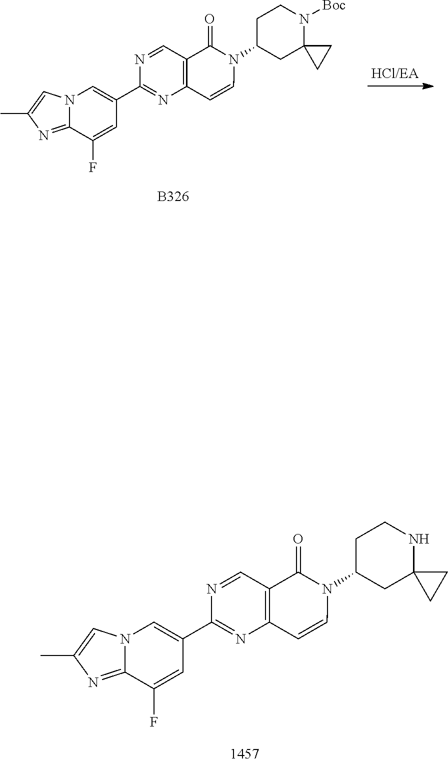 [SMILES: Cc1cn2cc(-c3ncc4c(=O)n([C@@H]5CCN(C(=O)OC(C)(C)C)C6(CC6)C5)ccc4n3)cc(F)c2n1 ; "B326" ; Cc1cn2cc(-c3ncc4c(=O)n([C@@H]5CCNC6(CC6)C5)ccc4n3)cc(F)c2n1]

To a solution of tert-butyl (7R)-7-[2-(8-fluoro-2-methyl-imidazo[1,2-a]pyridin-6-yl)-5-oxo-pyrido[4,3-d]pyrimidin-6-yl]-4-azaspiro[2.5]octane-4-carboxylate (74.0 mg, 146 umol, 1.00 eq) in ethyl acetate (0.70 mL) was added HCl/ethyl acetate (4.0 M, 0.70 mL). The reaction mixture was stirred at 20° C. for 1 h, then concentrated under reduced pressure to give a residue. The residue was washed with dichloromethane (1.00 mL), then dried in vacuum, diluted with water (0.50 mL), and lyophilized to give 6-[(7R)-4-azaspiro[2.5]octan-7-yl]-2-(8-fluoro-2-methyl-imidazo[1,2-a]pyridin-6-yl)pyrido[4,3-d]pyrimidin-5-one (56.0 mg, 87%) as a solid. LCMS: (ESI, m/z): 405.1 [M+H]⁺. ¹H NMR (400 MHz, D₂O) δ ppm 9.57 (s, 1H), 9.40 (s, 1H), 8.47 (d, J=10.76 Hz, 1H), 7.95-8.06 (m, 2H), 6.94 (d, J=7.70 Hz, 1H), 5.15-5.25 (m, 1H), 3.59-3.68 (m, 1H), 3.41 (br dd, J=6.72, 3.42 Hz, 1H), 2.68-2.76 (m, 1H), 2.54 (s, 3H), 2.25-2.35 (m, 2H), 1.64 (br dd, J=13.69, 3.30 Hz, 1H), 1.16 (br s, 2H), 0.93-1.04 (m, 2H). ¹⁹F NMR (376 MHz, D₂O) δ ppm−132.097 (s, 1F).

### Synthesis of Compound 1458

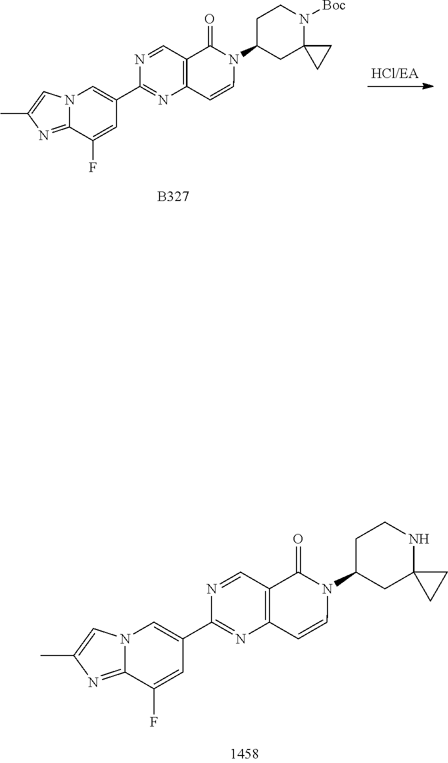 [SMILES: Cc1cn2cc(-c3ncc4c(=O)n([C@H]5CCN(C(=O)OC(C)(C)C)C6(CC6)C5)ccc4n3)cc(F)c2n1 ; "B327" ; Cc1cn2cc(-c3ncc4c(=O)n([C@H]5CCNC6(CC6)C5)ccc4n3)cc(F)c2n1]

To a solution of tert-butyl (7R)-7-[2-(8-fluoro-2-methyl-imidazo[1,2-a]pyridin-6-yl)-5-oxo-pyrido[4,3-d]pyrimidin-6-yl]-4-azaspiro[2.5]octane-4-carboxylate (74.0 mg, 146 umol, 1.00 eq) in ethyl acetate (0.70 mL) was added HCl/ethyl acetate (4.0 M, 0.70 mL). The reaction mixture was stirred at 20° C. for 1 h, then concentrated under reduced pressure to give a residue. The residue was washed with dichloromethane (1.00 mL), then dried in vacuum, diluted with water (0.50 mL), and lyophilized to give 6-[(7S)-4-azaspiro[2.5]octan-7-yl]-2-(8-fluoro-2-methyl-imidazo[1,2-a]pyridin-6-yl)pyrido[4,3-d]pyrimidin-5-one (56.0 mg, 87%) as a solid. LCMS: (ESI, m/z): 405.1 [M+H]⁺. ¹HNMR (400 MHz, D₂O) δ ppm 9.44 (br s, 1H), 9.03-9.32 (m, 1H), 8.11-8.34 (m, 1H), 7.93 (br d, J=7.09 Hz, 2H), 6.85 (br d, J=3.18 Hz, 1H), 5.07-5.21 (m, 1H), 3.65 (br dd, J=8.01, 2.38 Hz, 1H), 3.34-3.45 (m, 1H), 2.70 (br t, J=13.08 Hz, 1H), 2.45 (br s, 3H), 2.30 (br s, 2H), 1.59-1.66 (m, 1H), 1.18 (br d, J=1.83 Hz, 2H), 0.99 (br s, 2H). ¹⁹F NMR (376 MHz, D₂O) δ ppm−132.107 (s, 1F).

### Example G153: Synthesis of Compounds 645 and 646

### Synthesis of Intermediate B328

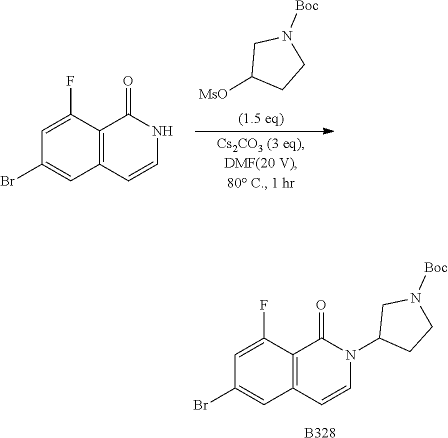

To a mixture of 6-bromo-8-fluoro-2H-isoquinolin-1-one (500 mg, 2.07 mmol, 1 eq) and tert-butyl 3-methylsulfonyloxypyrrolidine-1-carboxylate (822 mg, 3.10 mmol, 1.5 eq) in dimethyl formamide (10.0 mL) was added Cs₂CO₃ (2.02 g, 6.20 mmol, 3 eq) at 25° C. under N₂ atmosphere. The reaction mixture was stirred at 80° C. for 1 h, then filtered and the filtrate was concentrated under reduced pressure to give a residue. The residue was purified by silica gel column chromatography (ethyl acetate) to give tert-butyl 3-(6-bromo-8-fluoro-1-oxo-2-isoquinolyl)pyrrolidine-1-carboxylate (400 mg, 47%) as a solid. LCMS: (ES, m/z): 355.0 [M-tBu+H]⁺. ¹H NMR (400 MHz, DMSO-d₆) δ ppm 7.78 (s, 1H) 7.53 (br dd, J=11.21, 1.79 Hz, 2H) 6.65 (dd, J=7.57, 1.85 Hz, 1H) 5.22-5.30 (m, 1H) 3.63-3.74 (m, 1H) 3.44-3.56 (m, 2H) 3.37-3.42 (m, 1H) 2.15-2.24 (m, 2H) 1.40 (s, 9H).

### Synthesis of Intermediate B329

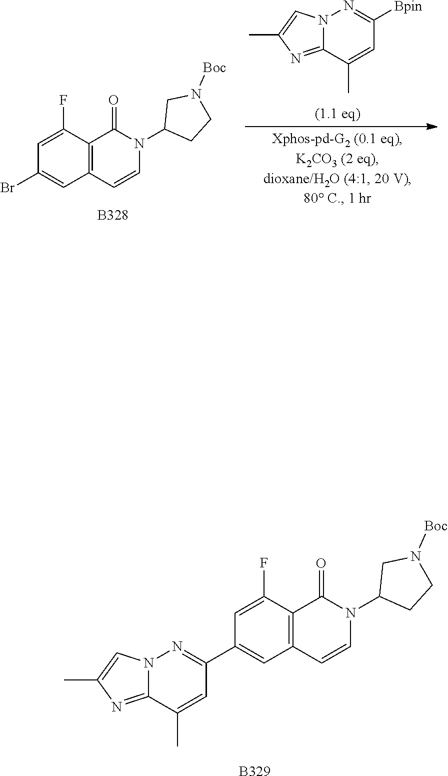

To a mixture of tert-butyl 3-(6-bromo-8-fluoro-1-oxo-2-isoquinolyl)pyrrolidine-1-carboxylate (400 mg, 972 umol, 1 eq) and 2,8-dimethyl-6-(4,4,5,5-tetramethyl-1,3,2-dioxaborolan-2-yl)imidazo[1,2-b]pyridazine (292 mg, 1.07 mmol, 1.1 eq) in dioxane (6.40 mL) and water (1.60 mL) was added K₃PO₄ (413 mg, 1.95 mmol, 2 eq) and Xphos-Pd-G₂ (76.5 mg, 97.3 umol, 0.1 eq) at 25° C. under N₂ atmosphere. The reaction mixture was stirred at 80° C. for 1 h, then cooled to 25° C. The resulting mixture was filtered and washed with dichloromethane (3×3.00 mL). The organic layers were combined and concentrated under reduced pressure to give a residue. The residue was purified by silica gel column chromatography (ethyl acetate/methanol=10/1) to give tert-butyl 3-[6-(2,8-dimethylimidazo[1,2-b]pyridazin-6-yl)-8-fluoro-1-oxo-2-isoquinolyl]pyrrolidine-1-carboxylate (250 mg, 54%) as a solid. LCMS: (ES, m/z): 478.2 [M+H]⁺. ¹H NMR (400 MHz, DMSO-d₆) δ ppm 8.17 (d, J=1.35 Hz, 1H) 8.10 (s, 1H) 7.81-7.87 (m, 1H) 7.78 (d, J=0.98 Hz, 1H) 7.52 (br s, 1H) 6.79 (dd, J=7.58, 1.83 Hz, 1H) 5.33 (br d, J=6.85 Hz, 1H) 3.72 (br d, J=7.46 Hz, 1H) 3.52 (dq, J=7.86, 5.45 Hz, 1H) 3.35-3.40 (m, 2H) 3.17 (d, J=5.26 Hz, 1H) 2.69 (s, 3H) 2.42 (s, 3H) 2.19-2.25 (m, 2H) 1.42 (br s, 9H). ¹⁹F NMR (376 MHz, DMSO-d₆) δ ppm−109.791 (s, 1F).

### Synthesis of Intermediate B330

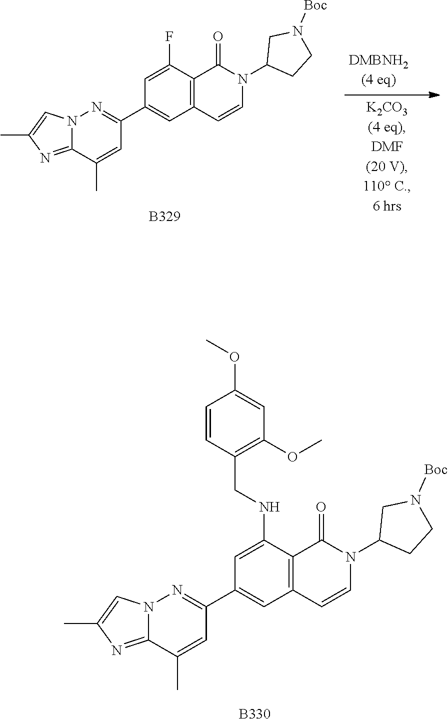 [SMILES: Cc1cn2nc(-c3cc(F)c4c(=O)n(C5CCN(C(=O)OC(C)(C)C)C5)ccc4c3)cc(C)c2n1 ; "B329" ; COc1ccc(CNc2cc(-c3cc(C)c4nc(C)cn4n3)cc3ccn(C4CCN(C(=O)OC(C)(C)C)C4)c(=O)c23)c(OC)c1 ; "B330"]

To a solution of tert-butyl 3-[6-(2,8-dimethylimidazo[1,2-b]pyridazin-6-yl)-8-fluoro-1-oxo-2-isoquinolyl]pyrrolidine-1-carboxylate (42-2, 300 mg, 628 umol, 1 eq) in dimethyl formamide (6.00 mL) was added K₂CO₃ (347 mg, 2.51 mmol, 4 eq) and (2,4-dimethoxyphenyl)methanamine (420 mg, 2.51 mmol, 379 uL, 4 eq) at 25° C. under N₂ atmosphere. The reaction mixture was stirred at 110° C. for 6 h, then poured into water (30.0 mL) and extracted with ethyl acetate (3×30.0 mL). The organic layers were combined, washed with brine (100 mL), dried with anhydrous Na₂SO₄, filtered, and the filtrate concentrated under reduced pressure to give a residue. The residue was purified by silica gel column chromatography (ethyl acetate/methanol=8/1) to give tert-butyl 3-[8-[(2,4-dimethoxyphenyl)methylamino]-6-(2,8-dimethylimidazo[1,2-b]pyridazin-6-yl)-1-oxo-2-isoquinolyl]pyrrolidine-1-carboxylate (350 mg, 89%) as a solid. LCMS: (ES, m/z): 625.3 [M+H]⁺. ¹H NMR (400 MHz, DMSO-d₆) δ ppm 9.51 (br t, J=5.07 Hz, 1H) 8.08 (s, 1H) 7.62 (s, 1H) 7.35 (br s, 1H) 7.24-7.30 (m, 2H) 7.13 (s, 1H) 6.61-6.65 (m, 2H) 6.52 (dd, J=8.38, 2.26 Hz, 1H) 5.27-5.37 (m, 1H) 4.40 (br d, J=5.14 Hz, 2H) 3.84 (s, 3H) 3.75 (s, 3H) 3.66-3.71 (m, 2H) 3.47-3.52 (m, 2H) 2.61 (s, 3H) 2.41 (s, 3H) 2.16-2.24 (m, 2H) 1.41 (br s, 9H).

### Synthesis of Intermediate B331

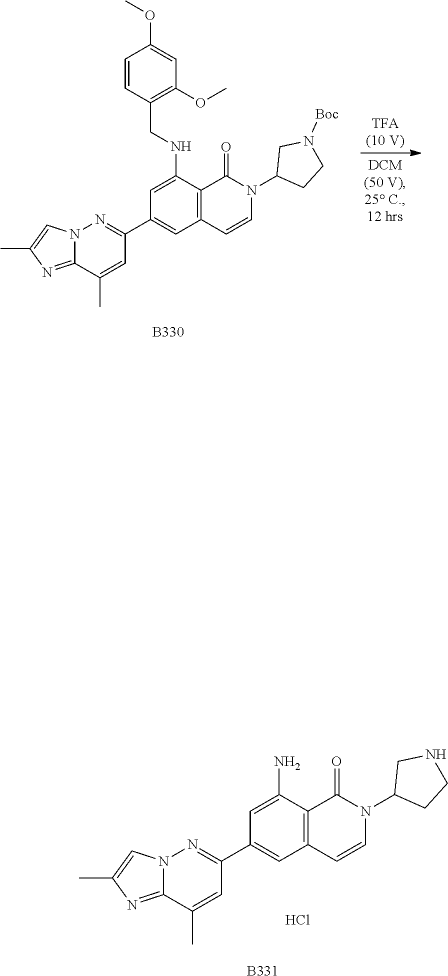 [SMILES: COc1ccc(CNc2cc(-c3cc(C)c4nc(C)cn4n3)cc3ccn(C4CCN(C(=O)OC(C)(C)C)C4)c(=O)c23)c(OC)c1 ; "B330" ; Cc1cn2nc(-c3cc(N)c4c(=O)n(C5CCNC5)ccc4c3)cc(C)c2n1 ; Cl ; "B331"]

To a solution of tert-butyl 3-[8-[(2,4-dimethoxyphenyl)methylamino]-6-(2,8-dimethylimidazo[1,2-b]pyridazin-6-yl)-1-oxo-2-isoquinolyl]pyrrolidine-1-carboxylate (150 mg, 240 umol, 1 eq) in dichloromethane (7.50 mL) was added 2,2,2-trifluoroacetate (1.50 mL) at 25° C. The reaction mixture was stirred at 25° C. for 12 h, then filtered and the filter cake concentrated under reduced pressure to give a residue. The residue was purified by prep-HPLC (column: Phenomenex Luna 80×30 mm×3 um; mobile phase: [water (HCl)-acetonitrile]; B %: 1%-30%, 8 min) to give 8-amino-6-(2,8-dimethylimidazo[1,2-b]pyridazin-6-yl)-2-pyrrolidin-3-yl-isoquinolin-1-one (37.0 mg, 41%) as a solid. LCMS: (ES, m/z): 375.1 [M+H]⁺. ¹H NMR (400 MHz, DMSO-d₆) δ ppm 9.13-9.39 (m, 2H) 8.39 (s, 1H) 7.99-8.12 (m, 1H) 7.49 (d, J=7.58 Hz, 1H) 7.32 (dd, J=8.62, 1.53 Hz, 2H) 6.61 (d, J=7.46 Hz, 1H) 5.24-5.35 (m, 1H) 3.13-3.32 (m, 4H) 2.71 (s, 3H) 2.54 (s, 3H) 2.41 (td, J=8.62, 4.65 Hz, 1H) 2.19-2.28 (m, 1H).

### Synthesis of Compound 645 and 646

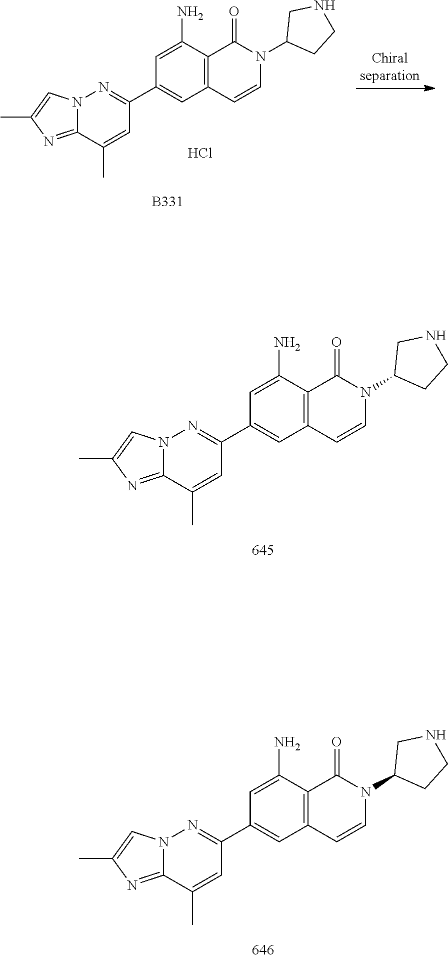

8-amino-6-(2,8-dimethylimidazo[1,2-b]pyridazin-6-yl)-2-pyrrolidin-3-yl-isoquinolin-1-one (37.0 mg, 98.8 umol, 1 eq) was purified by SFC (column: DAICEL CHIRALCEL OZ 250×25 mm I.D. 10 um; mobile phase: [0.1% NH₃H₂O methanol]; B %: 60%-60%, 30 min) to give 8-amino-6-(2,8-dimethylimidazo[1,2-b]pyridazin-6-yl)-2-[(3S)-pyrrolidin-3-yl]isoquinolin-1-one (11.2 mg, 30%) and 8-amino-6-(2,8-dimethylimidazo[1,2-b]pyridazin-6-yl)-2-[(3R)-pyrrolidin-3-yl]isoquinolin-1-one (7.76 mg, 21%) as solids. 645: LCMS: (ES, m/z): 375.1 [M+H]⁺. ¹H NMR (400 MHz, METHANOL-d₄) δ ppm 7.90 (s, 1H) 7.53 (d, J=0.83 Hz, 1H) 7.25-7.28 (m, 3H) 6.58 (d, J=7.63 Hz, 1H) 5.21-5.34 (m, 1H) 3.29 (br s, 2H) 2.99-3.11 (m, 2H) 2.66 (d, J=0.72 Hz, 3H) 2.49 (s, 3H) 2.30-2.38 (m, 1H) 2.00-2.12 (m, 1H). 646: LCMS: (ES, m/z): 375.1 [M+H]⁺. ¹H NMR (400 MHz, METHANOL-d₄) δ ppm 7.89 (s, 1H) 7.52 (s, 1H) 7.23-7.27 (m, 3H) 6.57 (d, J=7.51 Hz, 1H) 5.21-5.32 (m, 1H) 3.30 (br s,2H) 2.99-3.12 (m, 2H) 2.66 (s, 3H) 2.49 (s, 3H) 2.31-2.40 (m, 1H) 2.01-2.11 (m, 1H).

### Example 193: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (I)). After treatment, the levels of the HTT, MYB, or SMN2 mRNA targets were determined from each sample of cell lysate by cDNA synthesis followed by qPCR.

### Materials:

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_m1)—used for K562/suspension cell lines
GUSB: VIC-PL, ThermoFisher 4326320E (Assay: Hs99999908_m1)—used for K562/suspension cell lines
PPIA: VIC-PL, ThermoFisher 4326316E (Assay: Hs99999904_m1)—used for A673/adherent cell lines

Probe/primer sequences
Canonical junction
(CJ)
HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/
Alternative junction (AJ)
HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/
3IABKFQ/
MYB Primer 1:
CAACACCATTTCATAGAGACCAGAC
MYB Primer 2:
GTTCTAAAATCATCCCTTGGCTTCTAAT
MYB FAM-Probe:
/56-FAM/AAATACTGT/ZEN/ATAGGACCTCTTCTGACA
TCC/3IABKFQ/

### Description

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO₂ for 24 hours.

The cells were then gently washed with 50 uL-100 uL cold PBS before proceeding to addition of lysis buffer. 30 uL-50 uL of room temperature lysis buffer with DNAse I (and optionally RNAsin) was added to each well. Cells were shaken/mixed thoroughly at room temperature for 5-10 minutes for lysis to take place and then 3 uL-5 uL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3× lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 uL. The table below summarizes the components of the RT-qPCR reactions:

| Component | 1X |
|---|---|
| Tagman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time | |
|---|---|---|---|---|
| RT step | 1 | 50° C. | 5 | min |
| RT inactivation/initial | 1 | 95° C. | 20 | sec |
| denaturation | | | | |
| Amplification | 40 | 95° C. | 3 | sec |
| — | | 60° C. | 30 | sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 A673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 5000 response in AJ increase) while the decrease in CJ mRNA levels is reported as IC₅₀ (compound concentration having 50% response in CJ decrease).

A summary of these results is illustrated in Table 4, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 μM; and “D” represents an AC₅₀/IC₅₀ of greater than 10 μM.

TABLE 4
Modulation of RNA Splicing by Exemplary Compounds

| | HTT | HTT | MYB | MYB |
|---|---|---|---|---|
| | AJ | CJ | AJ | CJ |
| Cmpd | AC₅₀ | IC₅₀ | AC₅₀ | IC₅₀ |
| No. | (nM) | (nM) | (nM) | (nM) |
| 108 | D | D | D | D |
| 111 | C | C | C | C |
| 115 | D | D | C | B |
| 121 | D | D | D | C |
| 152 | D | D | C | D |
| 153 | D | D | D | D |
| 156 | C | C | C | C |
| 157 | D | D | C | C |
| 158 | D | D | D | D |
| 159 | D | D | D | D |
| 160 | D | D | D | D |
| 161 | D | D | D | D |
| 162 | D | D | D | D |
| 163 | D | D | C | D |
| 165 | D | D | — | — |
| 166 | C | C | C | C |
| 167 | D | D | D | D |
| 172 | D | D | D | D |
| 173 | D | D | D | D |
| 174 | B | B | B | B |
| 175 | D | C | C | C |
| 176 | C | C | B | C |
| 177 | D | D | D | D |
| 178 | D | D | D | D |
| 179 | D | D | D | D |
| 180 | D | D | D | D |
| 181 | B | B | B | B |
| 182 | C | C | B | B |
| 185 | B | C | C | C |
| 186 | A | A | A | B |
| 188 | C | C | C | B |
| 190 | B | B | B | B |
| 191 | D | D | D | D |
| 192 | D | D | D | D |
| 203 | D | D | D | D |
| 204 | D | D | D | D |
| 205 | B | B | B | B |
| 206 | B | B | B | A |
| 207 | C | C | C | C |
| 208 | D | C | C | C |
| 209 | C | B | B | B |
| 210 | C | C | C | C |
| 215 | C | C | C | C |
| 216 | C | C | B | C |
| 217 | C | C | C | C |
| 218 | C | C | C | C |
| 219 | C | C | B | B |
| 220 | D | D | D | D |
| 221 | D | D | D | D |
| 222 | C | C | C | C |
| 223 | C | C | B | B |
| 224 | D | D | D | C |
| 225 | D | D | D | D |
| 226 | D | D | C | C |
| 227 | C | C | C | C |
| 228 | B | B | B | B |
| 229 | D | D | D | D |
| 230 | A | A | A | A |
| 231 | B | B | B | B |
| 232 | A | A | A | A |
| 233 | B | B | B | B |
| 234 | C | C | C | C |
| 235 | D | D | D | D |
| 236 | B | B | B | A |
| 237 | B | B | B | B |
| 238 | D | D | D | D |
| 239 | D | D | D | C |
| 241 | C | C | C | C |
| 242 | A | A | A | A |
| 243 | A | A | A | A |
| 244 | D | D | D | D |
| 245 | B | B | B | B |
| 246 | C | C | C | C |
| 247 | C | C | B | B |
| 248 | A | B | B | B |
| 249 | C | C | C | C |
| 250 | A | A | B | B |
| 251 | B | B | B | B |
| 252 | D | D | C | C |
| 253 | C | C | C | C |
| 254 | C | C | C | C |
| 255 | C | C | C | C |
| 256 | B | B | B | B |
| 257 | B | B | B | B |
| 258 | C | D | — | — |
| 259 | B | B | B | B |
| 260 | B | B | C | B |
| 261 | D | D | D | D |
| 262 | C | C | C | B |
| 263 | D | D | D | D |
| 264 | C | C | C | C |
| 265 | A | A | B | A |
| 266 | A | A | B | A |
| 267 | C | B | C | A |
| 268 | C | C | C | B |
| 269 | D | D | D | D |
| 270 | C | C | C | C |
| 271 | C | C | C | C |
| 272 | A | B | A | B |
| 273 | A | B | — | — |
| 274 | A | A | A | A |
| 276 | A | A | A | A |
| 277 | B | B | B | B |
| 278 | A | A | B | B |
| 279 | B | B | A | B |
| 280 | B | B | B | B |
| 281 | C | C | C | C |
| 282 | D | D | D | D |
| 283 | B | B | B | B |
| 284 | D | D | D | D |
| 285 | C | D | B | C |
| 286 | C | C | C | C |
| 287 | B | B | B | B |
| 288 | C | C | C | C |
| 290 | B | B | B | B |
| 291 | B | B | B | A |
| 292 | B | B | B | B |
| 293 | B | C | B | B |
| 294 | C | C | B | B |
| 295 | C | C | B | B |
| 296 | B | B | A | A |
| 297 | C | C | B | B |
| 298 | B | B | B | B |
| 299 | D | D | D | D |
| 300 | B | C | C | B |
| 301 | C | C | C | C |
| 302 | C | C | C | C |
| 303 | C | C | C | C |
| 304 | C | C | B | B |
| 305 | B | C | | |
| 306 | C | C | C | D |
| 308 | C | C | C | C |
| 309 | A | A | A | A |
| 310 | B | B | A | A |
| 311 | C | C | C | C |
| 312 | A | A | A | A |
| 313 | D | D | | |
| 314 | C | C | C | C |
| 315 | C | C | B | B |
| 316 | B | B | A | A |
| 317 | B | B | A | B |
| 318 | A | A | A | A |
| 319 | C | C | B | C |
| 320 | D | D | D | D |
| 321 | D | D | C | C |
| 322 | A | A | A | A |
| 323 | B | B | B | B |
| 327 | C | C | C | C |
| 337 | B | B | B | B |
| 338 | B | C | A | A |
| 339 | C | D | C | C |
| 340 | B | B | A | A |
| 341 | B | B | B | A |
| 342 | A | B | A | A |
| 343 | B | B | A | A |
| 344 | C | C | B | B |
| 345 | A | A | A | A |
| 346 | B | B | A | A |
| 347 | D | D | C | C |
| 348 | D | D | D | D |
| 349 | B | B | B | B |
| 350 | A | A | A | A |
| 351 | A | A | A | A |
| 352 | D | D | D | D |
| 353 | B | B | B | B |
| 354 | C | C | C | C |
| 355 | B | A | A | A |
| 356 | B | A | B | A |
| 357 | A | A | A | A |
| 358 | C | D | C | C |
| 359 | C | C | A | C |
| 360 | D | D | C | D |
| 361 | A | A | A | A |
| 362 | D | D | C | C |
| 363 | D | D | D | D |
| 364 | D | D | D | D |
| 365 | D | D | C | C |
| 366 | C | C | C | C |
| 367 | B | B | B | B |
| 368 | A | A | B | B |
| 369 | A | A | A | A |
| 370 | A | A | A | A |
| 371 | C | C | C | C |
| 372 | B | B | B | B |
| 373 | C | C | C | C |
| 374 | B | B | B | B |
| 375 | C | C | C | C |
| 376 | B | B | B | B |
| 377 | B | B | B | B |
| 378 | C | C | C | C |
| 379 | B | B | B | B |
| 380 | A | A | A | A |
| 381 | A | B | A | A |
| 382 | C | C | A | B |
| 383 | B | C | A | A |
| 384 | A | A | A | A |
| 385 | D | D | B | C |
| 386 | C | C | C | C |
| 387 | A | A | A | A |
| 388 | D | D | C | C |
| 389 | B | B | B | B |
| 390 | A | A | A | A |
| 391 | A | A | A | A |
| 392 | C | C | A | A |
| 393 | B | C | B | B |
| 394 | A | A | A | A |
| 395 | C | C | A | B |
| 396 | C | C | A | A |
| 397 | A | A | B | A |
| 398 | A | A | A | A |
| 399 | B | B | B | B |
| 400 | A | A | A | A |
| 402 | B | B | A | B |
| 403 | C | C | C | B |
| 404 | C | C | C | C |
| 405 | D | D | C | B |
| 406 | B | B | A | A |
| 407 | B | B | A | B |
| 408 | B | B | A | A |
| 409 | D | D | D | D |
| 410 | C | C | B | B |
| 411 | C | C | C | C |
| 412 | A | A | A | A |
| 413 | A | A | A | A |
| 414 | C | C | C | C |
| 415 | A | A | A | A |
| 416 | B | B | A | A |
| 417 | A | A | A | A |
| 418 | A | A | A | A |
| 419 | D | D | C | C |
| 420 | C | C | B | B |
| 421 | D | D | D | D |
| 422 | B | B | A | A |
| 423 | A | A | A | A |
| 424 | B | B | A | B |
| 425 | A | A | A | A |
| 426 | A | A | A | A |
| 427 | D | D | D | C |
| 428 | C | C | B | B |
| 429 | D | D | D | D |
| 430 | D | D | D | D |
| 431 | A | A | A | A |
| 432 | C | C | A | B |
| 433 | D | D | B | C |
| 434 | D | D | B | B |
| 435 | B | B | A | A |
| 436 | B | B | A | A |
| 437 | D | D | C | C |
| 438 | C | C | B | C |
| 439 | D | D | C | C |
| 440 | B | B | A | A |
| 441 | D | D | C | C |
| 442 | D | D | D | D |
| 444 | B | B | A | A |
| 445 | A | A | A | A |
| 446 | A | A | A | A |
| 447 | A | A | A | A |
| 448 | A | A | A | A |
| 449 | D | D | D | D |
| 450 | C | D | C | C |
| 452 | D | D | C | C |
| 453 | D | D | D | D |
| 454 | D | D | D | D |
| 456 | D | D | D | D |
| 461 | B | B | A | A |
| 462 | C | C | C | C |
| 471 | D | D | D | D |
| 472 | B | B | B | B |
| 499 | B | B | B | B |
| 500 | B | B | B | B |
| 509 | D | D | D | D |
| 510 | B | C | C | C |
| 511 | B | B | B | B |
| 512 | B | C | C | C |
| 513 | B | B | C | C |
| 514 | A | A | A | A |
| 515 | B | B | C | B |
| 516 | C | C | C | C |
| 517 | C | C | B | B |
| 518 | D | D | C | D |
| 519 | A | A | A | A |
| 520 | B | B | B | B |
| 521 | C | C | C | C |
| 522 | D | D | D | D |
| 523 | D | C | C | C |
| 524 | D | D | D | D |
| 525 | A | B | B | A |
| 526 | B | B | B | B |
| 527 | B | B | B | B |
| 528 | B | C | C | C |
| 529 | B | C | B | B |
| 530 | D | D | D | D |
| 531 | A | B | B | B |
| 532 | B | B | B | B |
| 533 | B | B | B | B |
| 534 | C | C | C | C |
| 535 | C | C | C | C |
| 536 | D | D | D | D |
| 537 | A | A | B | B |
| 538 | A | A | B | B |
| 539 | A | A | A | A |
| 540 | A | A | A | A |
| 541 | A | A | A | A |
| 542 | A | A | A | A |
| 543 | A | B | B | B |
| 544 | A | A | B | A |
| 545 | A | A | A | A |
| 546 | A | A | A | A |
| 547 | A | A | A | A |
| 548 | A | A | A | A |
| 549 | A | A | A | B |
| 550 | A | A | A | A |
| 551 | B | B | B | B |
| 552 | D | D | D | D |
| 553 | A | A | B | B |
| 554 | B | B | B | B |
| 555 | A | A | B | B |
| 556 | B | B | B | B |
| 557 | B | B | B | B |
| 558 | C | C | B | B |
| 559 | A | B | A | A |
| 560 | B | C | B | B |
| 561 | B | B | C | B |
| 585 | C | C | C | C |
| 586 | B | C | B | B |
| 587 | B | B | A | A |
| 588 | C | C | B | C |
| 589 | D | D | C | C |
| 637 | A | A | A | A |
| 638 | A | A | A | B |
| 640 | B | C | B | B |
| 641 | B | C | A | B |
| 642 | B | C | B | B |
| 643 | D | D | C | C |
| 644 | D | D | B | C |
| 645 | C | D | C | C |
| 646 | D | D | D | C |
| 647 | C | D | C | C |
| 648 | D | D | D | D |
| 649 | C | C | C | C |
| 650 | D | D | D | D |
| 651 | A | B | A | A |
| 652 | D | D | C | C |
| 653 | D | D | C | C |
| 654 | A | A | A | A |
| 655 | B | C | A | B |
| 656 | B | B | A | A |
| 1123 | A | B | A | A |
| 1158 | B | C | B | B |
| 1181 | A | A | A | A |
| 1182 | B | B | B | B |
| 1191 | A | B | A | A |
| 1196 | B | B | A | A |
| 1198 | B | B | B | B |
| 1199 | C | C | C | B |
| 1204 | B | B | B | B |
| 1205 | B | B | B | B |
| 1206 | D | D | B | C |
| 1207 | C | C | C | C |
| 1208 | D | D | C | C |
| 1209 | B | B | B | B |
| 1210 | D | D | C | B |
| 1211 | B | B | B | A |
| 1212 | B | B | A | B |
| 1214 | D | D | C | C |
| 1215 | D | D | B | C |
| 1219 | C | C | C | C |
| 1220 | B | B | B | B |
| 1221 | B | B | B | B |
| 1222 | C | D | B | B |
| 1223 | C | C | B | B |
| 1225 | B | B | B | B |
| 1227 | D | D | C | C |
| 1228 | D | D | B | C |
| 1231 | B | B | B | B |
| 1238 | D | D | D | C |
| 1240 | D | D | C | C |
| 1241 | B | B | A | A |
| 1242 | A | A | A | A |
| 1243 | D | D | D | D |
| 1246 | B | B | A | A |
| 1247 | A | A | A | A |
| 1283 | B | B | B | B |
| 1284 | A | B | A | A |
| 1285 | A | A | A | A |
| 1286 | A | A | B | A |
| 1287 | A | A | A | A |
| 1288 | A | A | A | A |
| 1289 | A | A | A | A |
| 1290 | A | A | A | A |
| 1291 | A | A | A | A |
| 1292 | B | B | B | B |
| 1293 | D | D | D | D |
| 1294 | C | C | B | B |
| 1296 | A | B | A | A |
| 1297 | B | B | B | B |
| 1298 | A | A | B | B |
| 1299 | D | D | B | C |
| 1300 | B | C | B | B |
| 1301 | B | B | B | B |
| 1302 | D | D | D | D |
| 1303 | B | B | B | A |
| 1304 | B | C | B | B |
| 1305 | D | D | C | C |
| 1306 | B | B | B | B |
| 1307 | C | C | C | C |
| 1308 | A | B | B | B |
| 1309 | A | B | B | B |
| 1310 | B | C | C | C |
| 1311 | B | B | B | B |
| 1428 | C | D | B | B |
| 1429 | B | B | A | A |
| 1431 | C | C | C | C |
| 1432 | C | C | B | B |
| 1434 | B | C | B | B |
| 1435 | C | C | B | B |
| 1436 | C | C | A | A |
| 1437 | C | C | B | B |
| 1438 | C | C | B | B |
| 1439 | C | D | B | B |
| 1440 | D | D | B | B |
| 1441 | B | B | A | A |
| 1442 | B | C | B | A |
| 1443 | C | C | B | B |
| 1444 | B | B | B | B |
| 1445 | D | D | C | D |
| 1446 | D | D | C | C |
| 1447 | B | B | B | B |
| 1448 | B | C | B | B |
| 1449 | C | C | B | B |
| 1450 | C | C | B | B |
| 1451 | B | B | A | B |
| 1452 | B | B | A | B |
| 1453 | C | D | B | C |
| 1454 | B | C | B | B |
| 1455 | B | B | A | A |
| 1456 | D | D | B | B |
| 1457 | B | C | B | B |
| 1458 | B | B | A | B |
| 1459 | B | B | A | B |
| 1460 | B | C | B | B |
| 1461 | B | B | A | A |
| 1462 | B | B | A | A |
| 1463 | B | C | B | B |
| 1464 | B | B | A | B |
| 1465 | B | B | B | B |
| 1466 | B | B | B | B |
| 1467 | C | D | B | B |
| 1468 | B | B | A | A |
| 1469 | C | C | B | B |
| 1470 | C | D | B | B |
| 1471 | B | B | A | A |
| 1472 | C | C | B | B |
| 1473 | C | D | B | B |
| 1474 | C | C | B | C |
| 1475 | C | D | C | C |
| 1476 | B | C | B | B |
| 1477 | A | B | A | A |
| 1478 | C | C | B | B |
| 1479 | C | C | A | B |
| 1480 | B | B | B | B |
| 1481 | B | C | A | B |
| 1482 | C | C | A | B |
| 1483 | B | C | B | C |
| 1484 | A | B | A | A |
| 1485 | B | B | B | B |
| 1486 | B | C | A | B |
| 1488 | B | C | B | B |
| 1489 | C | C | B | B |
| 1490 | A | B | A | A |
| 1491 | B | B | B | B |
| 1492 | C | C | B | B |
| 1493 | C | C | B | B |
| 1494 | C | C | B | B |
| 1495 | A | B | A | A |
| 1496 | B | C | B | B |
| 1497 | B | C | B | B |
| 1498 | C | C | B | A |
| 1499 | A | A | A | A |
| 1500 | B | B | A | A |
| 1501 | B | B | A | A |
| 1502 | C | C | B | B |
| 1503 | B | B | B | A |
| 1504 | B | C | A | A |
| 1505 | B | B | B | B |
| 1506 | A | A | B | A |
| 1507 | B | B | B | B |
| 1508 | A | A | A | A |
| 1509 | B | B | A | B |
| 1510 | B | C | B | C |
| 1511 | B | B | B | B |
| 1512 | B | B | A | B |
| 1513 | B | C | B | B |
| 1514 | D | C | B | B |
| 1515 | B | C | A | B |
| 1516 | C | C | B | B |
| 1517 | B | B | B | B |
| 1518 | B | B | B | A |
| 1519 | A | B | B | B |
| 1520 | A | B | A | A |
| 1521 | B | C | B | B |
| 1522 | B | B | A | A |
| 1523 | B | C | B | A |
| 1524 | B | B | B | A |
| 1525 | C | C | A | A |
| 1526 | C | D | A | A |
| 1527 | B | B | B | B |
| 1528 | B | B | B | B |
| 1530 | A | A | A | A |
| 1531 | A | B | B | B |
| 1533 | A | B | B | B |
| 1534 | A | A | B | A |
| 1536 | B | B | B | B |
| 1537 | B | B | B | B |
| 1538 | B | B | B | B |
| 1540 | B | B | B | B |
| 1541 | A | B | B | B |
| 1542 | B | B | C | C |
| 1543 | A | A | B | B |
| 1544 | B | B | B | B |
| 1545 | A | A | B | B |
| 1547 | B | C | C | C |
| 1548 | B | B | B | B |
| 1549 | B | B | C | C |
| 1550 | B | B | B | B |
| 1551 | B | B | B | B |
| 1552 | B | B | B | B |
| 1553 | B | C | C | C |
| 2151 | D | D | B | D |
| 2154 | D | D | D | D |
| 2155 | D | D | D | D |
| 2164 | D | D | D | D |
| 2168 | D | D | D | D |
| 2169 | D | D | D | D |
| 2170 | D | D | D | D |
| 2171 | D | D | D | D |
| 2194 | C | C | C | C |
| 2195 | D | D | D | D |
| 2196 | D | D | D | D |
| 2197 | C | C | C | C |
| 2198 | D | D | D | D |
| 2199 | D | D | D | D |
| 2200 | D | D | D | D |
| 2201 | D | D | D | D |
| 2202 | C | C | C | C |
| 2211 | D | D | D | D |
| 2212 | C | C | C | C |
| 2213 | D | D | D | D |
| 2214 | D | D | | |

Additional studies were carried out for a larger panel of genes using the protocol provided above. The junction between flanking upstream and downstream exons was used to design canonical junction Qpcr assays. At least one of the forward primer, reverse primer or the CY5-labeled 5′ nuclease probe (with 3′ quencher such as ZEN/Iowa Black FQ) was designed to overlap with the exon junction to capture the C J Mrna transcript. BLAST was used to confirm the specificity of the probeset and parameters such as melting temperature, GC content, amplicon size, and primer dimer formation are considered during their design. Data for the decrease in CJ Mrna levels for four exemplary genes (HTT, SMN2, MYB, and Target C) analyzed in this panel are reported as IC₅₀ (compound concentration having 5000 response in CJ decrease).

A summary of the results from the panel is illustrated in Table 5, wherein “A” represents an IC₅₀ of less than 100 Nm; “B” represents an IC₅₀ of between 100 Nm and 1 μM; and “C” represents an IC₅₀ of between 1 μM and 10 μM; and “D” represents an IC₅₀ of greater than 10 μM.

TABLE 5
Modulation of RNA Splicing by Exemplary Compounds

| Cmpd | | | | |
|---|---|---|---|---|
| No. | HTT | SMN2 | Target C | MYB |
| 108 | D | C | C | D |
| 111 | C | B | C | C |
| 115 | D | A | D | C |
| 121 | C | B | D | D |
| 152 | D | D | D | D |
| 153 | D | C | — | C |
| 156 | C | B | — | C |
| 157 | D | C | — | C |
| 158 | D | D | — | D |
| 159 | D | D | — | D |
| 160 | D | D | D | D |
| 161 | D | D | D | D |
| 162 | D | D | — | D |
| 163 | D | D | D | C |
| 165 | D | B | — | — |
| 166 | C | C | C | C |
| 167 | D | D | D | D |
| 172 | D | D | D | D |
| 173 | D | C | — | D |
| 174 | B | A | — | B |
| 175 | C | C | D | C |
| 176 | C | B | C | B |
| 177 | D | D | C | D |
| 178 | D | D | D | D |
| 179 | D | C | D | D |
| 180 | D | D | C | D |
| 181 | B | A | B | B |
| 182 | C | A | C | B |
| 185 | B | B | — | C |
| 186 | A | A | B | B |
| 188 | C | A | C | C |
| 190 | B | B | B | B |
| 191 | D | D | D | D |
| 192 | D | D | D | D |
| 203 | D | D | D | D |
| 204 | D | D | D | D |
| 205 | B | A | B | B |
| 206 | B | A | B | B |
| 207 | C | B | C | C |
| 208 | D | B | D | C |
| 209 | C | A | C | B |
| 210 | C | B | C | C |
| 215 | C | A | C | C |
| 216 | C | A | C | B |
| 217 | C | B | D | C |
| 218 | C | C | C | C |
| 219 | C | A | C | B |
| 220 | D | D | D | D |
| 221 | D | B | D | D |
| 222 | C | B | C | C |
| 223 | C | B | D | B |
| 224 | D | B | D | D |
| 225 | D | D | D | D |
| 226 | D | B | D | C |
| 227 | C | B | D | C |
| 228 | B | B | B | B |
| 229 | D | D | D | D |
| 230 | A | A | A | A |
| 231 | B | A | C | B |
| 232 | A | A | B | A |
| 233 | B | A | C | B |
| 234 | C | A | D | C |
| 235 | D | B | D | D |
| 236 | B | A | B | B |
| 237 | B | A | C | B |
| 238 | D | D | D | D |
| 239 | D | C | D | D |
| 241 | C | C | C | C |
| 242 | A | A | B | A |
| 243 | A | A | A | A |
| 244 | D | B | D | D |
| 245 | B | A | B | B |
| 246 | C | C | C | C |
| 247 | C | A | C | B |
| 248 | A | A | B | B |
| 249 | C | C | C | C |
| 250 | A | A | B | B |
| 251 | B | A | B | B |
| 252 | D | B | D | C |
| 253 | C | B | C | C |
| 254 | C | C | C | C |
| 255 | C | B | D | C |
| 256 | B | A | D | B |
| 257 | B | A | C | B |
| 258 | — | 1 | — | |
| 259 | B | B | B | B |
| 260 | B | B | C | C |
| 261 | D | C | D | D |
| 262 | C | A | C | C |
| 263 | D | D | D | D |
| 264 | C | B | D | C |
| 265 | A | A | B | B |
| 266 | A | A | B | B |
| 267 | C | A | C | C |
| 268 | C | A | D | C |
| 269 | D | C | D | D |
| 270 | C | B | D | C |
| 271 | C | B | C | C |
| 272 | A | A | B | A |
| 273 | A | — | B | |
| 274 | A | A | A | A |
| 275 | D | — | D | — |
| 276 | A | A | A | A |
| 277 | B | A | C | B |
| 278 | A | A | B | B |
| 279 | B | A | B | A |
| 280 | B | A | C | B |
| 281 | C | B | C | C |
| 282 | D | C | D | D |
| 283 | B | A | B | B |
| 284 | D | C | D | D |
| 285 | C | A | D | B |
| 286 | C | B | C | C |
| 287 | B | A | B | B |
| 288 | C | C | C | C |
| 290 | B | B | B | B |
| 291 | B | A | B | B |
| 292 | B | A | C | B |
| 293 | B | A | C | B |
| 294 | C | A | C | B |
| 295 | C | A | D | B |
| 296 | B | A | B | A |
| 297 | C | A | C | B |
| 298 | B | A | C | B |
| 299 | D | D | D | D |
| 300 | B | A | C | C |
| 301 | C | C | C | C |
| 302 | C | B | C | C |
| 303 | C | B | C | C |
| 304 | B | A | C | B |
| 305 | B | — | B | |
| 306 | C | A | C | C |
| 308 | C | B | C | C |
| 309 | A | A | A | A |
| 310 | B | A | B | A |
| 311 | C | B | C | C |
| 312 | A | A | A | A |
| 313 | D | B | D | |
| 314 | C | B | D | C |
| 315 | C | A | C | B |
| 316 | B | A | B | A |
| 317 | B | A | B | A |
| 318 | A | A | B | A |
| 319 | C | A | C | B |
| 320 | D | C | D | D |
| 32 | D | B | D | C |
| 322 | A | A | A | A |
| 323 | B | A | B | B |
| 327 | C | C | C | C |
| 337 | B | B | B | B |
| 338 | B | A | C | A |
| 339 | C | B | D | C |
| 340 | B | A | B | A |
| 341 | B | A | C | B |
| 342 | A | A | B | A |
| 343 | B | A | B | A |
| 344 | C | A | C | B |
| 345 | A | A | A | A |
| 346 | B | A | B | A |
| 347 | D | D | C | B |
| 348 | D | D | D | D |
| 349 | B | A | C | B |
| 350 | A | A | B | A |
| 351 | A | A | A | A |
| 352 | D | D | D | D |
| 353 | B | A | C | B |
| 354 | C | C | C | C |
| 355 | B | A | B | A |
| 356 | B | A | B | B |
| 357 | A | A | A | A |
| 358 | C | B | D | C |
| 359 | C | A | D | A |
| 360 | D | C | D | C |
| 361 | A | A | B | A |
| 362 | D | B | D | C |
| 363 | D | C | D | D |
| 364 | D | D | D | D |
| 365 | D | B | D | C |
| 366 | C | C | C | C |
| 367 | B | B | B | B |
| 368 | A | A | B | B |
| 369 | A | A | A | A |
| 370 | A | A | B | A |
| 371 | C | C | C | C |
| 372 | B | A | C | B |
| 373 | C | B | C | C |
| 374 | B | A | C | B |
| 375 | C | C | C | C |
| 376 | B | B | B | B |
| 377 | B | A | B | B |
| 378 | C | B | C | C |
| 379 | B | B | C | B |
| 380 | A | A | A | A |
| 381 | A | A | C | A |
| 382 | C | A | D | A |
| 383 | B | A | C | A |
| 384 | A | A | B | A |
| 385 | D | A | D | B |
| 386 | C | A | C | C |
| 387 | A | A | B | A |
| 388 | D | B | D | C |
| 389 | B | B | B | B |
| 390 | A | A | A | A |
| 391 | A | A | A | A |
| 392 | C | A | C | A |
| 393 | B | A | C | B |
| 394 | A | A | B | A |
| 395 | C | A | D | A |
| 396 | C | A | D | A |
| 397 | A | A | B | B |
| 398 | A | A | A | A |
| 399 | B | A | C | B |
| 400 | A | A | B | A |
| 402 | B | A | B | A |
| 403 | C | A | C | C |
| 404 | C | A | D | C |
| 405 | D | B | D | C |
| 406 | B | A | B | A |
| 407 | B | A | B | A |
| 408 | B | A | C | A |
| 409 | D | D | D | D |
| 410 | C | A | D | B |
| 411 | C | B | D | C |
| 412 | A | A | A | A |
| 413 | A | A | B | A |
| 414 | C | B | D | C |
| 415 | A | A | B | A |
| 416 | B | A | B | A |
| 417 | A | A | B | A |
| 418 | A | A | B | A |
| 419 | D | B | D | C |
| 420 | C | A | C | B |
| 421 | D | D | D | D |
| 422 | B | A | B | A |
| 423 | A | A | B | A |
| 424 | B | A | B | A |
| 425 | A | A | A | A |
| 426 | A | A | A | A |
| 427 | D | C | D | D |
| 428 | C | A | C | B |
| 429 | D | D | D | D |
| 430 | D | B | D | D |
| 431 | A | A | B | A |
| 432 | C | A | C | A |
| 433 | D | A | B | A |
| 434 | D | A | D | B |
| 435 | B | A | C | A |
| 436 | B | A | B | A |
| 437 | D | B | D | C |
| 438 | C | B | C | B |
| 439 | D | B | D | C |
| 440 | B | A | B | A |
| 441 | D | B | D | C |
| 442 | D | C | D | D |
| 444 | B | A | B | A |
| 445 | A | A | A | A |
| 446 | A | A | A | A |
| 447 | A | A | A | A |
| 448 | A | A | A | A |
| 449 | D | D | D | D |
| 451 | D | B | D | C |
| 452 | D | A | D | C |
| 453 | D | D | D | D |
| 454 | D | C | D | D |
| 456 | D | C | D | D |
| 461 | B | A | B | A |
| 462 | C | B | D | C |
| 471 | D | B | D | D |
| 472 | B | A | B | B |
| 499 | B | B | B | B |
| 500 | B | B | B | B |
| 509 | D | C | D | D |
| 510 | B | A | C | C |
| 511 | B | A | C | B |
| 512 | B | B | C | C |
| 513 | B | B | C | C |
| 514 | A | A | A | A |
| 515 | B | A | C | C |
| 516 | C | C | C | C |
| 517 | C | A | C | B |
| 518 | D | B | D | C |
| 519 | A | A | A | A |
| 520 | B | A | C | B |
| 521 | C | C | C | C |
| 522 | D | C | D | D |
| 523 | D | B | D | C |
| 524 | D | D | D | D |
| 525 | A | A | B | B |
| 526 | B | A | C | B |
| 527 | B | A | C | B |
| 528 | B | A | C | C |
| 529 | B | A | C | B |
| 530 | D | D | D | D |
| 531 | A | A | B | B |
| 532 | B | A | C | B |
| 533 | B | A | C | B |
| 534 | C | A | C | C |
| 535 | C | A | D | C |
| 536 | D | C | D | D |
| 537 | A | A | B | B |
| 538 | A | A | B | B |
| 539 | A | A | B | A |
| 540 | A | A | A | A |
| 541 | A | A | A | A |
| 542 | A | A | B | A |
| 543 | A | A | B | B |
| 544 | A | A | A | B |
| 545 | A | A | A | A |
| 546 | A | A | A | A |
| 547 | A | A | A | A |
| 548 | A | A | A | A |
| 549 | A | A | B | A |
| 550 | A | A | A | A |
| 551 | B | B | B | B |
| 552 | D | D | D | D |
| 553 | A | A | B | B |
| 554 | B | A | B | B |
| 555 | A | A | B | B |
| 556 | B | A | C | B |
| 557 | B | A | C | B |
| 558 | C | A | C | B |
| 559 | A | A | B | A |
| 560 | B | A | C | B |
| 561 | B | B | C | C |
| 585 | C | B | C | C |
| 586 | B | A | C | B |
| 587 | B | A | B | A |
| 588 | C | A | B | B |
| 589 | D | B | D | C |
| 637 | A | A | A | A |
| 638 | A | A | A | A |
| 640 | B | A | C | B |
| 641 | B | A | C | A |
| 642 | B | A | C | B |
| 643 | D | B | D | C |
| 644 | D | B | D | B |
| 645 | C | B | D | C |
| 646 | D | B | D | D |
| 647 | C | B | D | C |
| 648 | D | B | D | D |
| 649 | C | B | D | C |
| 650 | D | C | D | D |
| 651 | A | A | B | A |
| 652 | D | B | D | C |
| 653 | D | B | D | C |
| 654 | A | A | B | A |
| 655 | B | A | C | A |
| 656 | B | A | B | A |
| 1123 | A | A | B | A |
| 1158 | B | A | C | B |
| 1181 | A | A | A | A |
| 1182 | B | B | B | B |
| 1191 | A | A | B | A |
| 1196 | B | A | B | A |
| 1198 | B | B | B | B |
| 1199 | C | A | C | C |
| 1204 | B | A | C | B |
| 1205 | B | A | B | B |
| 1206 | D | A | D | B |
| 1207 | C | A | C | C |
| 1208 | D | B | D | C |
| 1209 | B | B | B | B |
| 1210 | D | B | D | C |
| 1211 | B | A | C | B |
| 1212 | B | A | B | A |
| 1214 | D | B | D | C |
| 1215 | D | A | D | B |
| 1219 | C | B | D | C |
| 1220 | B | A | B | B |
| 1221 | B | B | B | B |
| 1222 | C | A | D | B |
| 1223 | C | A | C | B |
| 1225 | B | A | B | B |
| 1227 | D | B | D | C |
| 1228 | D | A | D | B |
| 1231 | B | A | B | B |
| 1238 | D | C | D | D |
| 1240 | D | B | D | C |
| 1241 | B | A | C | A |
| 1242 | A | A | B | A |
| 1243 | D | D | D | D |
| 1246 | B | A | C | A |
| 1247 | A | A | B | A |
| 1283 | B | A | C | B |
| 1284 | A | A | B | A |
| 1285 | A | A | A | A |
| 1286 | A | A | A | B |
| 1287 | A | A | A | A |
| 1288 | A | A | A | A |
| 1289 | A | A | A | A |
| 1290 | A | A | A | A |
| 1291 | A | A | B | A |
| 1292 | B | B | B | B |
| 1293 | D | D | D | D |
| 1294 | C | A | C | B |
| 1296 | A | A | B | A |
| 1297 | B | A | B | B |
| 1298 | A | A | B | B |
| 1299 | D | B | D | B |
| 1300 | B | A | C | B |
| 1301 | B | A | C | B |
| 1302 | D | C | D | D |
| 1303 | B | A | B | B |
| 1304 | B | A | C | B |
| 1305 | D | A | D | C |
| 1306 | B | A | B | B |
| 1307 | C | B | C | C |
| 1308 | A | A | B | B |
| 1309 | A | A | B | B |
| 1310 | B | B | C | C |
| 1311 | B | A | B | B |
| 1428 | C | A | C | B |
| 1429 | B | A | C | A |
| 1431 | C | B | C | C |
| 1432 | C | A | C | B |
| 1434 | B | A | C | B |
| 1435 | C | A | D | B |
| 1436 | C | A | C | A |
| 1437 | C | A | C | B |
| 1438 | C | A | C | B |
| 1439 | C | A | D | B |
| 1440 | D | A | D | B |
| 1441 | B | A | B | A |
| 1442 | B | A | C | B |
| 1443 | C | A | C | B |
| 1444 | B | A | B | B |
| 1445 | D | B | D | C |
| 1446 | D | B | D | C |
| 1447 | B | A | C | B |
| 1448 | B | A | C | B |
| 1449 | C | A | D | B |
| 1450 | C | A | D | B |
| 1451 | B | A | B | A |
| 1452 | B | A | B | A |
| 1453 | C | A | D | B |
| 1454 | B | A | C | B |
| 1455 | B | A | C | A |
| 1456 | D | B | D | B |
| 1457 | B | A | C | B |
| 1458 | B | A | B | A |
| 1459 | B | A | B | A |
| 1460 | B | A | C | B |
| 1461 | B | A | B | A |
| 1462 | B | A | B | A |
| 1463 | B | A | C | B |
| 1464 | B | A | C | A |
| 1465 | B | A | B | B |
| 1466 | B | A | B | B |
| 1467 | C | A | D | B |
| 1468 | B | A | B | A |
| 1469 | C | A | B | B |
| 1470 | C | A | C | B |
| 1471 | B | A | B | A |
| 1472 | C | B | C | B |
| 1473 | C | A | C | B |
| 1474 | C | A | C | B |
| 1475 | C | B | C | C |
| 1476 | B | A | C | B |
| 1477 | A | A | B | A |
| 1478 | C | A | C | B |
| 1479 | C | A | C | A |
| 1480 | B | B | B | B |
| 1481 | B | A | C | A |
| 1482 | C | A | B | A |
| 1483 | B | A | B | B |
| 1484 | A | A | B | A |
| 1485 | B | A | B | B |
| 1486 | B | A | C | A |
| 1488 | B | A | C | B |
| 1489 | C | A | C | B |
| 1490 | A | A | B | A |
| 1491 | B | A | B | B |
| 1492 | C | A | C | B |
| 1493 | C | A | C | B |
| 1494 | C | A | C | B |
| 1495 | A | A | B | A |
| 1496 | B | A | C | B |
| 1497 | B | A | C | B |
| 1498 | C | A | C | B |
| 1499 | A | A | B | A |
| 1500 | B | A | B | A |
| 1501 | B | A | B | A |
| 1502 | C | A | C | B |
| 1503 | B | A | B | B |
| 1504 | B | A | C | A |
| 1505 | B | | B | B |
| 1506 | A | | B | B |
| 1507 | B | | C | B |
| 1508 | A | | A | A |
| 1509 | B | | B | A |
| 1510 | B | | C | B |
| 1511 | B | | B | B |
| 1512 | B | | B | A |
| 1513 | B | | C | B |
| 1514 | D | | C | B |
| 1515 | B | | B | A |
| 1516 | C | | D | B |
| 1517 | B | A | C | B |
| 1518 | B | A | B | B |
| 1519 | A | A | B | B |
| 1520 | A | A | B | A |
| 1521 | B | A | C | B |
| 1522 | B | A | B | A |
| 1523 | B | A | C | B |
| 1524 | B | A | B | B |
| 1525 | C | A | D | A |
| 1526 | C | A | D | A |
| 1527 | B | A | B | B |
| 1528 | B | B | B | B |
| 1530 | A | A | A | A |
| 1531 | A | A | B | B |
| 1533 | A | A | B | B |
| 1534 | A | A | A | B |
| 1536 | B | A | B | B |
| 1537 | B | A | B | B |
| 1538 | B | B | B | B |
| 1540 | B | A | B | B |
| 1541 | A | A | B | B |
| 1542 | B | B | C | C |
| 1543 | A | A | B | B |
| 1544 | B | B | B | B |
| 1545 | A | A | B | B |
| 1547 | B | B | C | C |
| 1548 | B | B | B | B |
| 1549 | B | B | C | C |
| 1550 | B | B | B | B |
| 1551 | B | A | B | B |
| 1552 | B | — | B | B |
| 1553 | B | B | C | C |
| 2151 | D | D | — | B |
| 2154 | D | D | — | D |
| 2155 | D | D | — | D |
| 2164 | D | D | C | D |
| 2168 | D | D | D | D |
| 2169 | D | D | D | D |
| 2170 | D | D | D | D |
| 2171 | D | D | D | D |
| 2194 | C | C | C | C |
| 2195 | D | D | D | D |
| 2196 | D | D | D | D |
| 2197 | C | C | C | C |
| 2198 | D | D | D | D |
| 2199 | D | D | D | D |
| 2200 | D | D | D | D |
| 2201 | D | D | D | D |
| 2202 | C | C | C | C |
| 2211 | D | D | D | D |
| 2212 | C | C | C | C |
| 2213 | D | D | D | D |
| 2214 | D | D | D | — |

### Example X: Evaluating Effect of Exemplary Compounds on Protein Abundance

Compounds described herein were used to screen for effects on quantitative protein abundance using a HiBit assay system (Promega). Quantitative protein abundance was determined by measuring the protein levels of HiBit-tagged protein targets expressed in cell culture via luminescence using the Nano-Glo HiBiT Lytic Detection System, which uses a split complementation assay format to reconstitute NanoBiT enzyme to generate a luminescent signal. A protein abundance assay was developed such that endogenous protein targets could be modified with the HiBiT peptide tag and their abundance could be assessed after compound treatment. Briefly, K562 cell lines containing a HiBiT-modification were treated with various compounds described herein (e.g., compounds of Formulas (I), (II), (III), (IV), (V), (VI), (VII), (VIII), or (IX)). After treatment for 24 hours, the protein abundance of a specific target was determined by measuring luminescence.

### Materials:

Promega Nano-Glo HiBiT Lytic Detection System (cat #N3030)
Corning 384-well TC-treated microplates (cat #3570)
Synthego Engineered Cells Knock-In Clones

TABLE 5
Design of genetically modified HiBiT cell lines

| | | | Guide | Guide | |
|---|---|---|---|---|---|
| Cell | | Modifi- | RNA | RNA cut | |
| Line | Gene | cation | Sequence | location | Donor Sequence |
| K562 | MYB | HIBIT | GCGCCA | chr6: | CGGTGCGGTCCCCGCGGCTC |
| | | | TGGCCC | 135,181,526 | TCGGCGGAGCCCCGCGCCCG |
| | | | GAAGAC | | CCGCGCCATGgtgagcggct |
| | | | CC | | ggcggctgttcaagaagatt |
| | | | | | agcGGCAGCTCCGGAGGATC |
| | | | | | TAGCGGCGCCCGAAGACCCC |
| | | | | | GGCACAGgtaacggggagcc |
| | | | | | gggcgggcggccgaggg |
| K562 | HTT | HIBIT | CAGCTTT | chr4: | CGAGTCGGCCCGAGGCCTCC |
| | | | TCCAGG | 3,074,830 | GGGGACTGCCGTGCCGGGCG |
| | | | GTCGCC | | GGAGACCGCCATGgtgagcg |
| | | | A | | gctggcggctgttcaagaag |
| | | | | | attagcGGCAGCTCCGGAGG |
| | | | | | ATCTAGCGGCGCGACCCTGG |
| | | | | | AAAAGCTGATGAAGGCCTTC |
| | | | | | GAGTCCCTCAAGTCCTTCCA |

### Description:

Cells were maintained in IMDM with 10% FBS. Before the assay, cells were diluted with phenolphthalein-free growth media (IMDM+100 FBS media) and were seeded in a 384-well plate at a density of 10,000 cells/well (for each cell line listed in Table 5). Each compound was prepared as a 10-point 3-fold serial dilution in DMSO with the top dose at a final concentration of 10 μM in the well. Unmodified K562 cells were added at the previously specified density with DMSO to serve as an assay baseline and positive control (PC) and DMSO only with the respective modified cell lines was added to the negative control (NC) columns. Final DMSO concentration was kept at or below 0.25%. Treated cell plates were placed in an incubator at 37° C. with 500 CO₂ for 24 hours. After 24 hours, 25 μL of Complete HiBit Lytic reagent was added to each well at room temperature (e.g. one plate requiring 10 mL Lytic Buffer, 100 μL LgBiT Protein, 200 μL Lytic Substrate), shaken for 5 minutes at 600 RPM, then left to sit for 10 minutes for signal to stabilize before reading on a Spark Cyto plate reader (Tecan) with a 500 ms measurement time.

To determine compound effects on protein abundance of each target in Table 5, the percent response for each respective cell line was calculated at each compound concentration as follows:

$$\% response = 100 * ( S - PC ) / ( NC - PC )$$

For the normalized response at each concentration, a four-parameter logistical regression was fit to the data and the response was interpolated at the 500% value to determine a concentration for protein abundance at 5000 (IC₅₀) the untreated control. A summary of the results for protein abundance can be prepared and labeled, with compounds following in the following representative categories: A represents <100 nM; B represents 100-1000 nM; C represents 1000-9999 nM; and D represents greater than 10 μM.

TABLE 12
Effect of exemplary compounds on protein abundance

| Cmpd | | Target | | | | | |
|---|---|---|---|---|---|---|---|
| No. | MYB | C | HTT | | | | |
| 186 | B | B | B | 264 | C | D | C |
| 188 | B | C | C | 265 | B | B | A |
| 215 | B | C | B | 266 | B | B | A |
| 216 | B | C | B | 267 | B | C | B |
| 217 | B | D | B | 268 | B | D | C |
| 218 | C | C | C | 269 | C | C | D |
| 219 | B | D | B | 270 | B | D | C |
| 220 | D | D | D | 271 | C | C | C |
| 221 | D | D | D | 272 | B | B | A |
| 222 | C | C | C | 273 | | | |
| 223 | C | D | C | 274 | A | A | A |
| 224 | D | D | D | 275 | | | |
| 225 | C | D | C | 276 | A | A | A |
| 226 | C | D | C | 277 | B | B | B |
| 247 | B | C | B | 278 | A | A | A |
| 248 | A | B | A | 279 | B | A | A |
| 249 | C | C | C | 280 | B | B | B |
| 250 | A | B | A | 281 | B | C | C |
| 251 | B | B | B | 282 | C | D | D |
| 252 | C | D | D | 283 | B | B | A |
| 253 | C | C | C | 304 | B | B | B |
| 254 | C | C | C | 305 | | | |
| 255 | D | D | D | 309 | A | A | A |
| 256 | B | B | B | 310 | B | B | B |
| 257 | B | B | B | 312 | A | A | A |
| 258 | | | | 322 | A | A | A |
| 259 | B | B | B | 324 | | | |
| 260 | B | C | B | 368 | A | A | A |
| 261 | D | D | D | 369 | C | C | B |
| 262 | B | C | C | 370 | A | A | A |
| 263 | D | C | D | 372 | B | C | B |
| | | | | 375 | C | C | C |
| | | | | 378 | C | C | C |
| 379 | B | B | B | 441 | C | D | C |
| 380 | A | A | A | 442 | D | D | D |
| 381 | A | B | A | 443 | | | |
| 382 | B | C | C | 444 | B | B | A |
| 383 | A | B | B | 445 | A | A | A |
| 384 | A | A | A | 446 | A | A | A |
| 387 | A | A | A | 447 | A | A | A |
| 390 | A | A | A | 448 | A | A | A |
| 391 | A | A | A | 449 | D | D | D |
| 392 | B | C | C | 451 | C | D | C |
| 393 | A | B | B | 452 | C | D | D |
| 394 | A | A | A | 453 | D | D | D |
| 395 | B | C | B | 454 | D | D | D |
| 396 | B | B | B | 456 | C | D | D |
| 397 | A | A | A | 457 | | | |
| 398 | A | A | A | 461 | A | B | A |
| 399 | B | B | B | 462 | B | C | C |
| 400 | A | A | A | 466 | A | A | A |
| 402 | A | B | A | 467 | B | B | B |
| 403 | B | C | C | 468 | A | A | A |
| 404 | B | C | C | 469 | A | A | A |
| 409 | D | D | D | 470 | B | A | A |
| 410 | B | C | B | 509 | D | D | D |
| 411 | C | C | C | 510 | C | C | B |
| 412 | A | A | A | 511 | B | B | B |
| 413 | A | A | A | 512 | C | A | C |
| 414 | C | D | C | 513 | C | C | B |
| 415 | A | A | A | 514 | A | A | A |
| 416 | A | A | A | 515 | C | C | B |
| 417 | A | A | A | 516 | C | C | C |
| 418 | A | A | A | 517 | B | C | C |
| 419 | C | D | D | 518 | C | C | C |
| 420 | B | C | C | 519 | A | A | A |
| 421 | D | D | D | 520 | B | C | B |
| 423 | A | A | A | 521 | C | C | C |
| 425 | A | A | A | 522 | C | D | D |
| 426 | A | A | A | 523 | C | C | C |
| 428 | B | C | B | 524 | D | D | D |
| 429 | C | B | D | 525 | A | B | A |
| 430 | C | D | D | 526 | A | B | B |
| 431 | A | A | A | 527 | B | B | B |
| 432 | A | C | C | 528 | C | B | B |
| 433 | C | D | D | 529 | B | B | B |
| 434 | C | D | D | 530 | D | D | D |
| 440 | A | B | B | 531 | A | B | A |
| 532 | B | B | B | 648 | C | D | D |
| 533 | A | B | B | 649 | C | C | C |
| 534 | B | C | C | 650 | D | D | D |
| 535 | C | C | C | 651 | A | B | A |
| 536 | D | D | D | 652 | C | D | D |
| 537 | B | B | A | 653 | C | D | D |
| 538 | B | B | A | 152 | D | D | D |
| 539 | A | A | A | 153 | D | D | D |
| 540 | A | A | A | 156 | C | D | C |
| 541 | A | A | A | 157 | C | C | C |
| 542 | A | A | A | 158 | C | C | D |
| 543 | B | B | A | 159 | D | D | D |
| 544 | A | A | A | 160 | D | D | D |
| 545 | A | A | A | 161 | D | D | D |
| 546 | A | A | A | 162 | D | D | D |
| 547 | A | A | A | 163 | D | D | D |
| 548 | A | A | A | 172 | D | D | D |
| 549 | A | A | A | 173 | D | D | D |
| 550 | A | A | A | 174 | A | B | B |
| 551 | B | B | B | 175 | C | D | C |
| 552 | C | D | D | 176 | B | C | C |
| 553 | B | B | A | 177 | D | D | D |
| 554 | B | B | A | 178 | D | D | D |
| 555 | B | B | A | 179 | D | D | D |
| 556 | B | B | A | 180 | D | D | D |
| 557 | B | B | A | 181 | A | A | A |
| 558 | B | C | B | 182 | B | C | C |
| 559 | A | B | A | 203 | D | D | D |
| 560 | B | B | B | 204 | D | D | D |
| 561 | B | B | B | 205 | B | B | B |
| 562 | B | B | B | 206 | B | B | B |
| 563 | B | B | B | 207 | C | C | C |
| 564 | B | B | B | 208 | C | D | C |
| 565 | A | A | A | 209 | D | D | D |
| 566 | B | B | B | 210 | C | C | C |
| 637 | A | A | A | 227 | C | D | C |
| 638 | B | A | A | 228 | B | B | B |
| 640 | A | C | C | 229 | D | D | D |
| 641 | A | B | B | 230 | A | B | B |
| 642 | B | C | B | 231 | B | B | B |
| 643 | C | D | D | 232 | A | B | A |
| 644 | C | D | D | 233 | B | B | B |
| 645 | C | D | C | 234 | C | C | C |
| 646 | C | D | D | 235 | C | D | D |
| 647 | C | D | C | 236 | B | C | B |
| 237 | B | C | B | 345 | D | D | D |
| 241 | C | C | C | 346 | A | B | B |
| 242 | A | A | A | 347 | C | D | D |
| 243 | A | A | A | 348 | D | D | D |
| 244 | D | D | D | 349 | B | C | B |
| 245 | B | B | B | 350 | A | A | B |
| 246 | C | C | C | 351 | A | A | A |
| 284 | C | D | D | 352 | D | D | D |
| 285 | B | C | C | 353 | B | C | B |
| 286 | B | C | C | 354 | C | C | C |
| 287 | B | B | B | 355 | A | A | A |
| 288 | C | C | C | 356 | A | A | A |
| 290 | B | B | B | 357 | A | A | A |
| 291 | B | B | B | 358 | C | C | C |
| 292 | B | C | B | 359 | B | C | B |
| 293 | B | C | B | 360 | D | D | D |
| 294 | B | C | B | 361 | A | A | A |
| 295 | C | C | C | 362 | C | D | D |
| 296 | B | B | B | 363 | C | C | D |
| 297 | B | C | C | 364 | D | D | D |
| 298 | B | C | B | 365 | C | D | D |
| 299 | D | D | D | 366 | C | C | C |
| 300 | B | C | B | 367 | B | B | B |
| 301 | C | C | C | 499 | B | B | B |
| 302 | C | C | C | 500 | B | B | B |
| 303 | C | C | C | 501 | A | A | A |
| 306 | C | C | B | 502 | D | D | D |
| 308 | C | C | C | 503 | A | B | A |
| 311 | C | C | C | 504 | D | D | D |
| 313 | C | D | D | 505 | B | B | B |
| 314 | C | D | C | 506 | D | D | D |
| 315 | B | C | C | 507 | C | C | D |
| 316 | A | B | B | 508 | C | D | D |
| 317 | A | B | B | 585 | C | C | C |
| 318 | A | B | A | 586 | B | C | B |
| 319 | B | C | C | 587 | A | B | B |
| 320 | D | D | D | 588 | B | C | C |
| 321 | D | D | D | 589 | C | D | C |
| 338 | B | C | C | 590 | B | C | C |
| 339 | D | D | D | 591 | C | D | D |
| 340 | A | B | B | | B | B | B |
| 341 | B | C | C | 1123 | A | A | A |
| 342 | B | B | B | 1191 | C | A | C |
| 343 | A | B | B | 1196 | A | B | B |
| 344 | B | D | C | 1198 | B | B | B |
| 1199 | C | C | C | 1303 | B | B | B |
| 1200 | | | | 1304 | B | C | B |
| 1204 | B | C | B | 1305 | B | D | D |
| 1205 | B | B | B | 1320 | A | B | B |
| 1206 | B | D | C | 1321 | B | C | B |
| 1207 | C | C | C | 1322 | C | C | D |
| 1208 | C | D | D | 1323 | A | D | C |
| 1209 | B | B | B | 1324 | B | C | C |
| 1210 | C | D | C | 1325 | B | C | B |
| 1211 | A | B | B | 1326 | A | B | B |
| 1212 | A | B | A | 1327 | B | D | D |
| 1213 | B | B | B | 1329 | A | B | B |
| 1214 | C | D | D | 1333 | B | C | B |
| 1215 | B | C | D | 1334 | B | C | B |
| 1218 | D | D | D | 1428 | A | C | C |
| 1219 | B | C | C | 1429 | A | B | B |
| 1220 | B | | B | 1431 | B | C | C |
| 1221 | B | B | B | 1432 | B | C | C |
| 1222 | A | D | D | 1434 | B | C | B |
| 1223 | B | C | C | 1436 | A | B | B |
| 1224 | A | B | A | 1437 | B | C | C |
| 1225 | B | B | B | 1438 | B | C | C |
| 1226 | A | A | A | 1439 | B | D | C |
| 1227 | C | D | D | 1440 | B | D | C |
| 1228 | B | C | D | 1441 | A | B | A |
| 1230 | A | B | A | 1442 | A | B | B |
| 1231 | B | B | B | 1443 | B | C | C |
| 1234 | A | A | A | 1444 | B | B | B |
| 1237 | A | A | A | 1445 | C | D | D |
| 1238 | D | D | D | 1446 | C | D | D |
| 1239 | A | A | A | 1447 | A | B | B |
| 1240 | D | D | D | 1448 | B | B | B |
| 1241 | A | A | A | 1449 | B | C | C |
| 1242 | A | A | A | 1450 | B | C | C |
| 1243 | D | D | D | 1451 | B | B | B |
| 1245 | A | A | A | 1452 | B | B | B |
| 1246 | B | B | B | 1453 | B | D | C |
| 1284 | A | B | A | 1454 | B | C | B |
| 1285 | A | A | A | 1455 | A | B | B |
| 1296 | A | B | A | 1456 | C | D | C |
| 1297 | B | B | B | 1457 | B | C | B |
| 1299 | C | D | D | 1458 | A | B | B |
| 1300 | B | C | B | 1459 | A | B | B |
| 1301 | B | B | B | 1460 | B | C | B |
| 1302 | D | D | D | 1461 | A | B | B |
| 1462 | A | B | A | 1537 | B | B | B |
| 1463 | B | C | B | 1538 | B | B | B |
| 1464 | A | B | B | 1540 | B | B | B |
| 1465 | B | B | B | 1541 | B | B | A |
| 1466 | B | B | B | 1542 | C | B | B |
| 1467 | B | C | B | 1543 | B | B | A |
| 1468 | A | B | B | 1545 | B | A | A |
| 1469 | B | C | C | 1553 | C | C | B |
| 1470 | B | C | C | 1158 | B | C | B |
| 1471 | B | B | B | 1181 | A | A | A |
| 1476 | B | C | B | 1182 | B | B | B |
| 1477 | B | B | A | 2151 | D | D | D |
| 1479 | A | C | B | 2154 | D | D | D |
| 1512 | A | B | A | 2155 | D | D | D |
| 1515 | A | B | B | 2164 | D | D | D |
| 1519 | A | A | A | 2168 | D | D | D |
| 1247 | A | A | A | 2169 | D | D | D |
| 1276 | B | A | A | 2170 | D | D | D |
| 1306 | B | B | B | 2171 | D | D | D |
| 1307 | C | C | C | 2195 | D | D | D |
| 1308 | B | B | A | 2196 | D | D | D |
| 1309 | B | B | B | 2197 | C | C | C |
| 1310 | C | C | C | 2198 | D | D | D |
| 1311 | B | B | B | 2199 | D | D | D |
| 1411 | C | C | C | 2200 | D | D | D |
| 1527 | B | B | B | 2201 | D | D | D |
| 1528 | B | B | B | 2202 | C | C | C |
| 1530 | A | A | A | 2211 | D | D | D |
| 1531 | B | B | B | 2212 | D | D | D |
| 1533 | B | B | B | 2213 | D | D | D |
| 1534 | B | A | A | 2214 | D | D | D |
| 1536 | B | B | B | | | | |

### Example X: Investigating Effect of Exemplary Compounds on Cell Viability

Compounds described herein were screened for toxicity in K562 (human chronic myelogenous leukemia) cells using a Cell Titer Glo 2.0 assay.

### Materials:

Promega CellTiter-Glo® 2.0 Cell Viability Assay (cat #G9241)
Corning 384-well TC-treated microplates (cat #3570)

### Description

Cells were plated at 500 cells/well (K562 cells) in 45 μL of IMDM supplemented with 1000 FBS in a 384-well opaque plate. Wells containing only medium were used as a blank control. Test compounds (e.g., compounds ofFormula (I), (II), (III), (IV), (V), (VI), (VII), (VIII), and (IX)) were first serially diluted in DMSO then diluted 1:100 with IMDM+10% FBS. The final concentration of DMSO was 0.1% in each well. The cells were incubated for 72 hours at 37° C. and 500 CO₂ before assaying with Cell Titer Glo 2.0 reagent.

Exemplary compounds were tested and found to fall within the following ranges: compounds labeled “A” represent <100 nM; “B” represent 100-1000 nM; “C” represent 1000-9999 nM; and “D” represent greater than 10 M in K562 cells.

| Compound | K562 | Compound | K562 | Compound | K562 |
|---|---|---|---|---|---|
| No. | GI₅₀ | No. | GI₅₀ | No. | GI₅₀ |
| 186 | B | 253 | B | 273 | |
| 188 | C | 254 | C | 274 | B |
| 215 | C | 255 | B | 275 | |
| 216 | B | 256 | A | 276 | A |
| 217 | C | 257 | A | 277 | C |
| 218 | C | 258 | | 278 | B |
| 219 | C | 259 | B | 279 | C |
| 220 | D | 260 | B | 280 | C |
| 221 | C | 261 | D | 281 | C |
| 222 | C | 262 | B | 282 | D |
| 223 | C | 263 | C | 283 | B |
| 224 | D | 264 | C | 304 | B |
| 225 | C | 265 | B | 305 | |
| 226 | C | 266 | B | 309 | A |
| 247 | B | 267 | C | 310 | C |
| 248 | A | 268 | C | 312 | B |
| 249 | C | 269 | D | 322 | A |
| 250 | A | 270 | C | 324 | |
| 251 | C | 271 | C | 368 | B |
| 252 | C | 272 | B | 369 | C |
| 370 | A | 412 | A | 449 | D |
| 372 | C | 413 | A | 451 | C |
| 375 | C | 414 | D | 452 | C |
| 378 | C | 415 | B | 453 | D |
| 379 | C | 416 | A | 454 | D |
| 380 | B | 417 | A | 456 | D |
| 381 | B | 418 | B | 457 | |
| 382 | C | 419 | D | 461 | A |
| 383 | B | 420 | C | 462 | C |
| 384 | B | 421 | D | 466 | A |
| 387 | A | 423 | B | 467 | C |
| 390 | A | 425 | A | 468 | B |
| 391 | A | 426 | A | 469 | A |
| 392 | C | 428 | C | 470 | B |
| 393 | B | 429 | C | 509 | D |
| 394 | A | 430 | C | 510 | C |
| 395 | B | 431 | B | 511 | C |
| 396 | B | 432 | C | 512 | C |
| 397 | B | 433 | C | 513 | C |
| 398 | A | 434 | C | 514 | A |
| 399 | B | 440 | A | 515 | C |
| 400 | B | 441 | C | 516 | C |
| 401 | | 442 | D | 517 | C |
| 402 | B | 443 | | 518 | C |
| 403 | C | 444 | B | 519 | A |
| 404 | C | 445 | A | 520 | C |
| 409 | D | 446 | B | 521 | C |
| 410 | C | 447 | B | 522 | C |
| 411 | C | 448 | A | 523 | C |
| 524 | D | 553 | B | 653 | D |
| 525 | B | 554 | B | 152 | C |
| 526 | B | 555 | B | 153 | |
| 527 | C | 556 | B | 156 | |
| 528 | C | 557 | B | 157 | |
| 529 | C | 558 | B | 158 | |
| 530 | D | 559 | A | 159 | |
| 531 | B | 560 | B | 160 | D |
| 532 | B | 561 | C | 161 | D |
| 533 | B | 562 | B | 162 | |
| 534 | C | 563 | B | 163 | D |
| 535 | C | 564 | B | 172 | C |
| 536 | D | 565 | B | 173 | C |
| 537 | B | 566 | B | 174 | B |
| 538 | B | 637 | B | 175 | C |
| 539 | B | 638 | B | 176 | C |
| 540 | A | 640 | B | 177 | D |
| 541 | A | 641 | B | 178 | D |
| 542 | B | 642 | C | 179 | D |
| 543 | B | 643 | D | 180 | D |
| 544 | B | 644 | C | 181 | B |
| 545 | A | 645 | C | 182 | B |
| 546 | A | 646 | C | 203 | D |
| 547 | A | 647 | C | 204 | D |
| 548 | A | 648 | C | 205 | B |
| 549 | B | 649 | C | 206 | B |
| 550 | A | 650 | C | 207 | C |
| 551 | B | 651 | A | 208 | C |
| 552 | D | 652 | C | 209 | C |
| 210 | B | 296 | B | 347 | D |
| 227 | C | 297 | C | 348 | D |
| 228 | B | 298 | C | 349 | C |
| 229 | D | 299 | D | 350 | B |
| 230 | B | 300 | C | 351 | B |
| 231 | B | 301 | C | 352 | D |
| 232 | B | 302 | C | 353 | B |
| 233 | B | 303 | C | 354 | C |
| 234 | C | 306 | B | 355 | B |
| 235 | C | 308 | C | 356 | B |
| 236 | B | 311 | C | 357 | A |
| 237 | B | 313 | C | 358 | C |
| 241 | C | 314 | C | 359 | B |
| 242 | B | 315 | C | 360 | C |
| 243 | A | 316 | B | 361 | B |
| 244 | D | 317 | B | 362 | C |
| 245 | B | 318 | B | 363 | C |
| 246 | C | 319 | B | 364 | D |
| 284 | D | 320 | D | 365 | D |
| 285 | B | 321 | D | 366 | C |
| 286 | B | 338 | B | 367 | B |
| 287 | B | 339 | D | 499 | B |
| 288 | C | 340 | B | 500 | B |
| 290 | | 341 | B | 501 | B |
| 291 | A | 342 | A | 502 | D |
| 292 | B | 343 | B | 503 | C |
| 293 | B | 344 | C | 504 | D |
| 294 | B | 345 | D | 505 | B |
| 295 | B | 346 | B | 506 | D |
| 507 | C | 1221 | B | 1303 | B |
| 508 | D | 1222 | C | 1304 | B |
| 585 | C | 1223 | C | 1305 | C |
| 586 | B | 1224 | A | 1320 | A |
| 587 | B | 1225 | B | 1321 | B |
| 588 | C | 1226 | A | 1322 | C |
| 589 | C | 1227 | C | 1323 | A |
| 590 | C | 1228 | C | 1324 | B |
| 591 | C | 1230 | A | 1325 | B |
| 1123 | A | 1231 | B | 1326 | B |
| 1191 | A | 1234 | A | 1327 | B |
| 1196 | B | 1237 | A | 1329 | B |
| 1198 | B | 1238 | D | 1333 | B |
| 1199 | C | 1239 | A | 1334 | B |
| 1204 | B | 1240 | C | 1428 | A |
| 1205 | B | 1241 | A | 1429 | B |
| 1206 | C | 1242 | A | 1431 | C |
| 1207 | C | 1243 | D | 1432 | B |
| 1208 | D | 1244 | | 1434 | B |
| 1209 | B | 1245 | B | 1435 | B |
| 1210 | C | 1246 | B | 1436 | A |
| 1211 | A | 1284 | A | 1437 | C |
| 1212 | B | 1285 | A | 1438 | C |
| 1213 | B | 1296 | B | 1439 | B |
| 1214 | C | 1297 | B | 1440 | B |
| 1215 | C | 1299 | C | 1441 | A |
| 1218 | D | 1300 | B | 1442 | B |
| 1219 | C | 1301 | B | 1443 | B |
| 1220 | B | 1302 | D | 1444 | B |
| 1445 | C | 1474 | B | 1504 | B |
| 1446 | B | 1475 | C | 1505 | C |
| 1447 | B | 1476 | B | 1506 | B |
| 1448 | B | 1477 | A | 1507 | B |
| 1449 | B | 1478 | B | 1508 | A |
| 1450 | B | 1479 | A | 1509 | B |
| 1451 | B | 1480 | B | 1510 | B |
| 1452 | B | 1481 | A | 1511 | B |
| 1453 | B | 1482 | A | 1512 | B |
| 1454 | B | 1483 | B | 1513 | B |
| 1455 | A | 1484 | A | 1514 | B |
| 1456 | C | 1485 | B | 1515 | B |
| 1457 | B | 1486 | A | 1516 | B |
| 1458 | B | 1488 | B | 1517 | C |
| 1459 | A | 1489 | B | 1518 | B |
| 1460 | B | 1490 | B | 1519 | B |
| 1461 | A | 1491 | B | 1520 | A |
| 1462 | A | 1492 | B | 1521 | B |
| 1463 | B | 1493 | B | 1522 | A |
| 1464 | A | 1494 | B | 1523 | B |
| 1465 | B | 1495 | B | 1524 | B |
| 1466 | B | 1496 | B | 1525 | A |
| 1467 | B | 1497 | B | 1526 | B |
| 1468 | A | 1498 | B | 1247 | B |
| 1469 | B | 1499 | B | 1276 | B |
| 1470 | B | 1500 | B | 1306 | B |
| 1471 | B | 1501 | B | 1307 | C |
| 1472 | C | 1502 | B | 1308 | B |
| 1473 | B | 1503 | B | 1309 | B |
| 1310 | B | 1544 | B | 2171 | D |
| 1311 | B | 1545 | B | 2194 | C |
| 1411 | C | 1547 | C | 2195 | D |
| 1527 | B | 1548 | B | 2196 | D |
| 1528 | B | 1549 | C | 2197 | C |
| 1530 | B | 1550 | C | 2198 | D |
| 1531 | B | 1551 | B | 2199 | D |
| 1533 | B | 1552 | B | 2200 | D |
| 1534 | B | 1553 | C | 2201 | D |
| 1536 | B | 1158 | B | 2202 | C |
| 1537 | B | 1181 | A | 2211 | D |
| 1538 | B | 1182 | B | 2212 | C |
| 1540 | B | 2164 | D | 2213 | C |
| 1541 | C | 2168 | D | 2214 | C |
| 1542 | C | 2169 | D | | |
| 1543 | B | 2170 | D | | |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, FIGURES, or Examples but rather is as set forth in the appended claims. Those of ordinary skill in the art will appreciate that various changes and modifications to this description may be made without departing from the spirit or scope of the present invention, as defined in the following claims.

1. A compound selected from one of
(i) Formula (I): 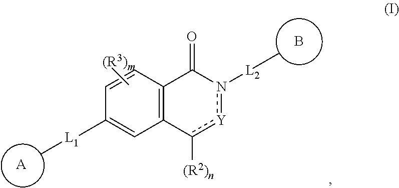
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁷;
Y is N, C(R^{6a}), or C(R^{6a})(R^{6b}), wherein the dashed lines in the ring comprising Y may be single or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, —NR^{B}C(O)R^{D}, or —S(O)ₓR^{D};
R³ is C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, C(O)NR^{B}R^{C}, —NR^{B}C(O)R^{D}, or —S(O)ₓR^{D};
R⁴ is hydrogen, C₁-C₆-alkyl, or C₁-C₆-haloalkyl;
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷;
R^{6a} and R^{6b} is independently hydrogen, C₁-C₆-alkyl, C₁-C₆-heteroalkyl, C₁-C₆-haloalkyl, or halo;
each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₂-C₆-alkenyl, C₂-C₆-alkynyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁹;
each R^{B} and R^{C} is independently hydrogen, C₁-C₆ alkyl, C₂-C₆-alkenyl, C₂-C₆-alkynyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —OR^{A}, —S(O)ₓR^{D}; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl, halo, or —OR^{A1};
each R^{A1} is hydrogen or C₁-C₆-alkyl;
each R⁹ and R¹⁰ is independently C₁-C₆-alkyl, halo, or —OR^{A1};
m is 0, 1, 2, or 3;
n is 0, 1, or 2; and
x is 0, 1, or 2;
or
(ii) Formula (II): 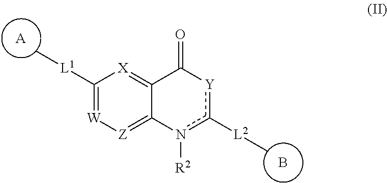
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
each of L¹ and L² is independently absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹;
each of W, X, and Z is independently C(R³) or N:
Y is N, N(R^{4a}), C(R^{4b}), or C(R^{4b})(R^{4c}), wherein the dashed lines in the ring comprising Y may be single or double bonds as valency permits:
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R² is absent, hydrogen, or C₁-C₆-alkyl;
R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}, or —S(O)ₓR^{D};
R^{4a} is hydrogen, C₁-C₆-alkyl, C₁-C₆-heteroalkyl, or C₁-C₆-haloalkyl;
each of R^{4b} and R^{4c} is independently hydrogen, C₁-C₆-alkyl, C₁-C₆-heteroalkyl, C₁-C₆-haloalkyl, halo, or —OR^{A};
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶;
each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R⁸ is independently hydrogen, C₁-C₆-alkyl, or C₁-C₆-haloalkyl;
each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R^{A} is independently hydrogen, C₁-C₆ alkyl, C₂-C₆-alkenyl, C₂-C₆-alkynyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁹;
each R^{B} and R^{C} is independently hydrogen, C₁-C₆ alkyl, C₂-C₆-alkenyl, C₂-C₆-alkynyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —OR^{A}, —S(O)ₓR^{D}, or R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R¹⁰ is independently C₁-C₆-alkyl or halo; and
x is 0, 1, or 2;
or
(iii) Formula (III): 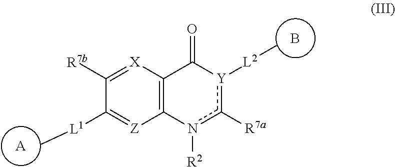
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
each of L¹ and L² is independently absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹;
each of X and Z is independently C(R³) or N;
Y is N, C, or C(R^{4b}), wherein the dashed lines in the ring comprising Y may be single or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R² is absent, hydrogen, or C₁-C₆-alkyl;
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, —C(O)OR^{D}, —S(O)ₓR^{D};
R^{4b} is hydrogen, C₁-C₆-alkyl, C₁-C₆-heteroalkyl, or C₁-C₆-haloalkyl;
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶;
each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
R^{7a} is hydrogen, C₁-C₆-alkyl, C₁-C₆-heteroalkyl, C₁-C₆-haloalkyl, halo, cyano, oxo, or —OR^{A};
R^{7b} is hydrogen, C₁-C₆-alkyl, C₁-C₆-heteroalkyl, C₁-C₆-haloalkyl, halo, cyano, or —OR^{A};
each R⁸ is independently hydrogen, C₁-C₆-alkyl, or C₁-C₆-haloalkyl;
each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each R^{B} and R^{C} is independently hydrogen, C₁-C₆ alkyl, C₁-C₆ heteroalkyl, cycloalkyl, heterocyclyl, —OR^{A}; or
R^{B} and R^{C} together with the atom to which they are attached form a 3-7-membered heterocyclyl ring optionally substituted with one or more R¹⁰;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R¹⁰ is independently C₁-C₆-alkyl or halo; and
x is 0, 1, or 2;
or
(iv) Formula (IV): 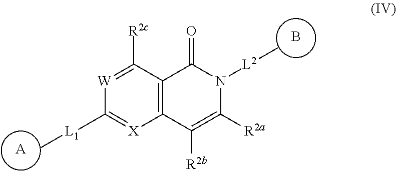
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
W and X are each independently C(R³) or N, wherein one of W and X is independently N;
each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁶;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R^{2a}, R^{2b}, and R^{2c} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D};
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D};
R⁴ is hydrogen, C₁-C₆-alkyl, or C₁-C₆-haloalkyl;
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷;
each R⁶ and R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —C(O)R^{D}, or —S(O)ₓR^{D};
each R^{B} and R^{C} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —OR^{A}, —S(O)ₓR^{D}; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁹ is independently C₁-C₆-alkyl or halo; and
x is 0, 1, or 2;
or
(v) Formula (V): 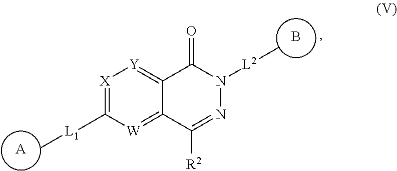
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
W, X, and Y are each independently C(R³) or N, wherein at least one of W, X, and Y is independently N;
each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁶;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R² is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D};
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D};
R⁴ is hydrogen, C₁-C₆-alkyl, or C₁-C₆-haloalkyl;
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷;
each R⁶ and R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —C(O)R^{D}, or —S(O)ₓR^{D};
each R^{B} and R^{C} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —OR^{A}, —S(O)ₓR^{D}; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁹ is independently C₁-C₆-alkyl or halo; and
x is 0, 1, or 2.

2. (canceled)

3. The compound of claim 1, wherein for Formula (I), (II), (III), (IV), or (V), one of A and B is independently a nitrogen-containing heterocyclyl, oxygen-containing heterocyclyl, or nitrogen-containing heteroaryl.

4. (canceled)

5. The compound of claim 1, wherein for Formulas (I), (II), (III), (IV), or (V), one of A and B is independently selected from 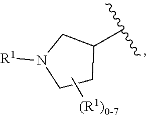
wherein R¹ is as described in claim 1.

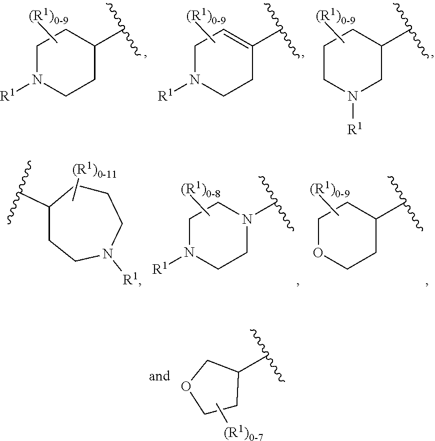

6-17. (canceled)

18. The compound of claim 1, wherein for Formulas (I), (II), (III), (IV), or (V), one of A and B is independently selected from 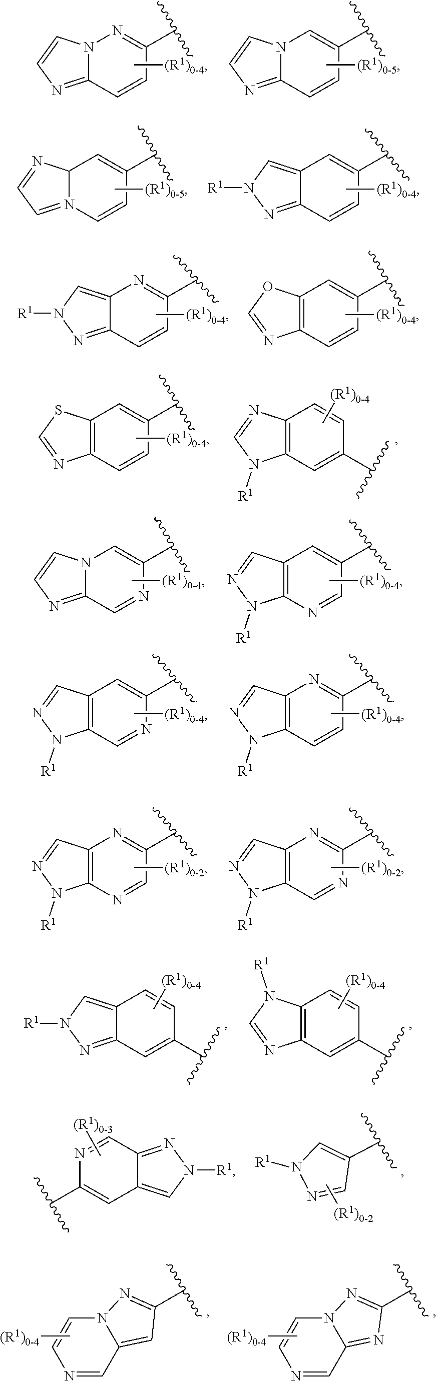 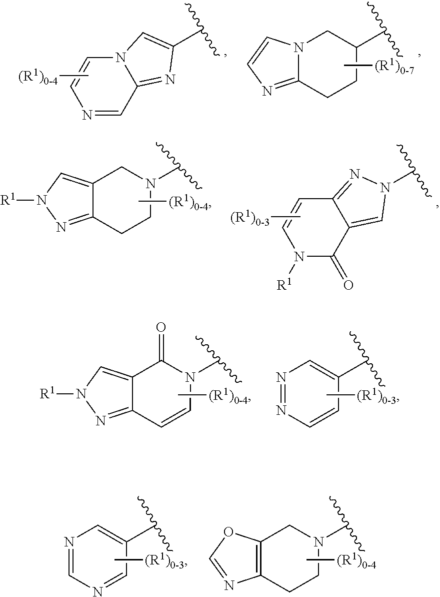
wherein R¹ is as described in claim 1.

19-25. (canceled)

26. The compound of claim 1, wherein for Formula (I):
(i) each of L¹ and L² is independently absent;
(ii) Y is C(R^{6a})(e.g., CH) or N;
(iii) R² is hydrogen or alkyl (e.g., CH₃);
(iv) n is 1 or 2; and/or
(v) m is 0 or 1.

27-34. (canceled)

35. The compound of claim 1, wherein for Formula (I), the compound of Formula (I) is a compound of:
(i) Formula (I-a): 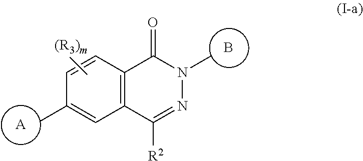
or a pharmaceutically acceptable salt, solvate, hydrate, tautomer, or stereoisomer thereof, wherein A, B, R², R³, and subvariables thereof are as defined in claim 1; or
(ii) Formula (I-b): 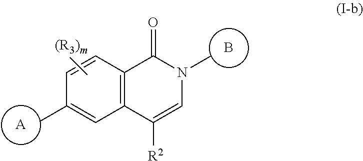
or a pharmaceutically acceptable salt, solvate, hydrate, tautomer, or stereoisomer thereof, wherein A, B, R², R³, and subvariables thereof are as defined in claim 1.

36-77. (canceled)

78. The compound of claim 1, wherein for Formula (II):
(i) each of L¹ and L² is independently absent;
(ii) W is C(R³)(e.g., CH);
(iii) X is C(R³)(e.g., CH);
(iv) Z is C(R³)(e.g., CH); and/or
(v) Y is N(R^{4a}) or C(R^{4b}), (e.g., NH).

79-83. (canceled)

84. The compound of claim 1, wherein for Formula (II), the compound of Formula (II) is a compound of:
(i) Formula (II-a): 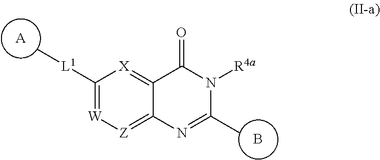
or a pharmaceutically acceptable salt, solvate, hydrate, tautomer, or stereoisomer thereof, wherein A, B, L¹, W, X, Z, R^{4a}, and subvariables thereof are as defined in claim 1;
(ii) Formula (II-b): 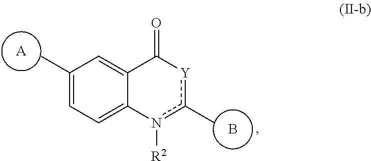
or a pharmaceutically acceptable salt, solvate, hydrate, tautomer, or stereoisomer thereof, wherein A, B, Y, R², and subvariables thereof are as defined in claim 1;
(iii) Formula (II-c): 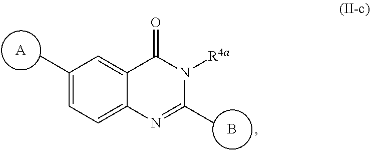
or a pharmaceutically acceptable salt, solvate, hydrate, tautomer, or stereoisomer thereof, wherein A, B, Y, R^{4a}, and subvariables thereof are as defined in claim 1; or
(iv) Formula (II-d): 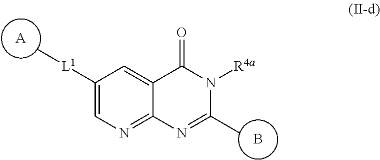
or a pharmaceutically acceptable salt, solvate, hydrate, tautomer, or stereoisomer thereof, wherein A, B, L¹, R^{4a}, and subvariables thereof are as defined in claim 1.

85-112. (canceled)

113. The compound of claim 1, wherein for Formula (III):
(i) each of L¹ and L² is independently absent,
(ii) X is C(R³)(e.g., CH);
(iii) Z is C(R³)(e.g., CH); and/or
(iv) Y is N.

114-122. (canceled)

123. The compound of claim 1, wherein for Formula (III), the compound of Formula (III) is a compound of:
(i) Formula (III-a): 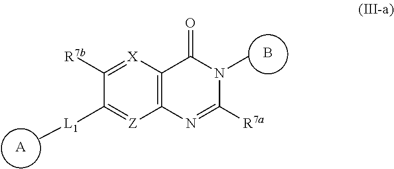
or a pharmaceutically acceptable salt, solvate, hydrate, tautomer, or stereoisomer thereof, wherein A, B, X, Z, R^{7b}, and subvariables thereof are as defined in claim 1;
(ii) Formula (III-b): 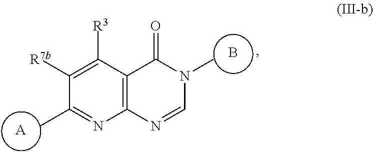
or a pharmaceutically acceptable salt, solvate, hydrate, tautomer, or stereoisomer thereof, wherein A, B, R³, R^{7b}, and subvariables thereof are as defined in claim 1; or
(iii) Formula (III-c): 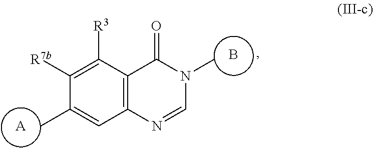
or a pharmaceutically acceptable salt, solvate, hydrate, tautomer, or stereoisomer thereof, wherein A, B, R³, R^{7b}, and subvariables thereof are as defined in claim 1.

124-144. (canceled)

145. The compound of claim 1, wherein for Formula (IV), one of A and B is selected from 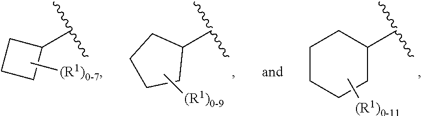
wherein R¹ is as defined in claim 1.

146-159. (canceled)

160. The compound of claim 1, wherein for Formula (IV):
(i) each of L¹ and L² is independently absent;
(ii) W is C(R³)(e.g., CH); or
(iii) W is N;
(iv) X is N; and/or
(v) each of W and X is N;
(vi) W is C(R³)(e.g., CH) and X is N; and/or
(vii) each of R^{2a}, R^{2b}, and R^{2c} is independently hydrogen or alkyl (e.g., CH₃).

161-171. (canceled)

172. The compound of claim 1, wherein for Formula (IV), the compound of Formula (IV) is a compound of:
(i) Formula (IV-a): 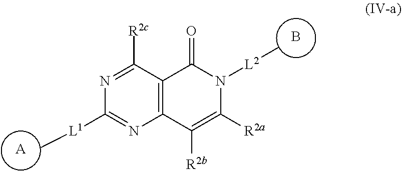
or a pharmaceutically acceptable salt, solvate, hydrate, tautomer, or stereoisomer thereof, wherein A, B, L¹, L², R^{2a}, R^{2b}, R^{2c}, and subvariables thereof are as defined in claim 1;
(ii) Formula (IV-d): 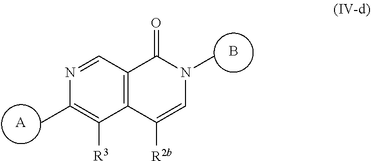
or a pharmaceutically acceptable salt, solvate, hydrate, tautomer, or stereoisomer thereof, wherein A, B, R^{2b}, R³, and subvariables thereof are as defined in claim 1; or
(iii) Formula (IV-e): 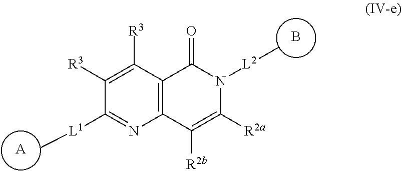
or a pharmaceutically acceptable salt, solvate, hydrate, tautomer, or stereoisomer thereof, wherein A, B, L¹, L², R^{2a}, R^{2b}, R^{2c}, R³, and subvariables thereof are as defined in claim 1.

173-215. (canceled)

216. The compound of claim 1, wherein for Formula (V):
(i) each of L¹ and L² is both absent;
(ii) W is C(R³)(e.g., CH); or
(iii) W is N;
(iv) X is C(R³)(e.g., CH); and/or
(v) Y is C(R³)(e.g., CH).

217-227. (canceled)

228. The compound of claim 1, wherein for Formula (V), the compound of Formula (V) is a compound of:
(i) Formula (V-a): 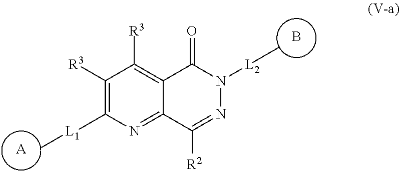
or a pharmaceutically acceptable salt, solvate, hydrate, tautomer, or stereoisomer thereof, wherein A, B, L¹, L², R², R³, and subvariables thereof are as defined in claim 1; or
(ii) Formula (V-e): 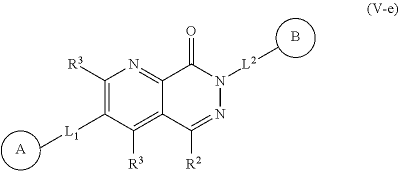
or a pharmaceutically acceptable salt, solvate, hydrate, tautomer, or stereoisomer thereof, wherein A, B, L¹, L², R^{2a}, R^{2b}, R^{2c}, R³, and subvariables thereof are as defined in claim 1.

229-239. (canceled)

240. The compound of claim 1, wherein the compound is a compound provided in one of Tables 1, 2, 3, 4, and.

241. A compound selected from one of:
(ii) Formula (VI): 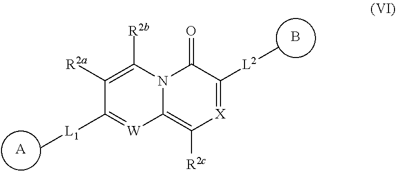
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
W and X are each independently C(R³) or N;
each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁶;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R^{2a}, R^{2b}, and R^{2c} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D};
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D};
R⁴ is hydrogen, C₁-C₆-alkyl, or C₁-C₆-haloalkyl;
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷;
each R⁶ and R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —C(O)R^{D}, or —S(O)ₓR^{D};
each R^{B} and R^{C} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —OR^{A}, S(O)ₓR^{D}; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁹ is independently C₁-C₆-alkyl or halo; and
x is 0, 1, or 2;
or
(ii) Formula (VII): 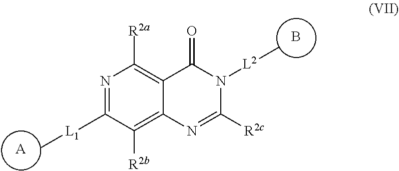
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁶;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R^{2a}, R^{2b}, and R^{2c} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, —OR^{A}, —NR^{B}R^{C}, —C(O)R^{D}, —C(O)OR^{D}, —C(O)NR^{B}R^{C}, or —S(O)ₓR^{D};
R⁴ is hydrogen, C₁-C₆-alkyl, or C₁-C₆-haloalkyl;
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷;
each R⁶ and R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —C(O)R^{D}, or —S(O)ₓR^{D};
each R^{B} and R^{C} is independently hydrogen, C₁-C₆ alkyl, C₁-C₆ heteroalkyl, C₁-C₆ haloalkyl, aryl, heteroaryl, C₁-C₆ alkylene-aryl, C₁-C₆ alkylene-heteroaryl, cycloalkyl, heterocyclyl, —OR^{A}, S(O)ₓR^{D}; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁹ is independently C₁-C₆-alkyl or halo; and
x is 0, 1, or 2.

242. (canceled)

243. The compound of claim 241, wherein for Formula (VI) or (VII), one of A and B is independently a nitrogen-containing heterocyclyl or nitrogen-containing heteroaryl.

244. The compound of claim 241, wherein for Formula (VI):
(i) one of A and B is independently selected from 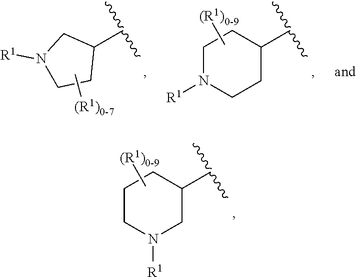
wherein R¹ is as described in claim 241; and/or
(ii) one of A and B is independently selected from 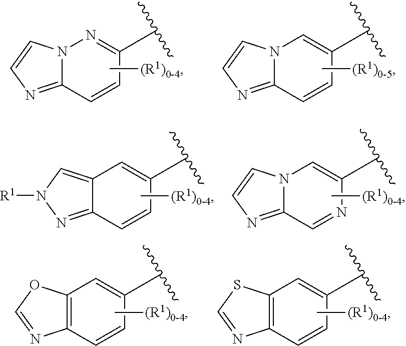
wherein R¹ is as described in claim 241.

245-267. (canceled)

268. The compound of claim 241, wherein for Formula (VI):
(i) each of L¹ and L² is independently absent; and/or
(ii)(a) each of W and X is N;
(b) W is N and X is C(R³)(e.g., CH);
(c) W is C(R³)(e.g., CH) and X is N; or
(d) W is C(R³)(e.g., CH) and X is C(R³)(e.g., CH); and/or
(iii) each of R^{2a}, R^{2b}, and R^{2c} is independently hydrogen.

269-280. (canceled)

281. The compound of claim 241, wherein for Formula (VI), the compound of Formula (VI) is a compound of;
(i) Formula (VI-a): 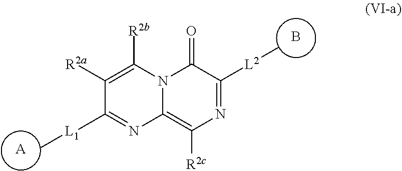
or a pharmaceutically acceptable salt, solvate, hydrate, tautomer, or stereoisomer thereof, wherein A, B, L¹, L², R^{2a}, R^{2b}, R^{2c}, and subvariables thereof are as defined in claim 241;
(ii) Formula (VI-b): 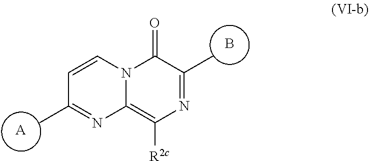
or a pharmaceutically acceptable salt, solvate, hydrate, tautomer, or stereoisomer thereof, wherein A, B, R^{2c}, and subvariables thereof are as defined in claim 241;
(iii) Formula (VI-c): 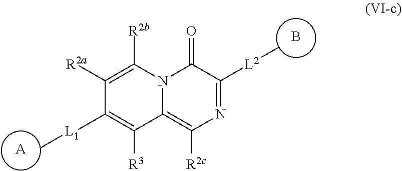
or a pharmaceutically acceptable salt, solvate, hydrate, tautomer, or stereoisomer thereof, wherein A, B, L¹, L², R^{2a}, R^{2b}, R^{2c}, R³, and subvariables thereof are as defined in claim 241;
(iv) Formula (VI-d): 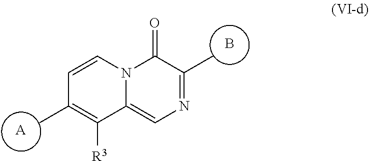
or a pharmaceutically acceptable salt, solvate, hydrate, tautomer, or stereoisomer thereof, wherein A, B, R³, and subvariables thereof are as defined in claim 241;
(v) Formula (VI-e): 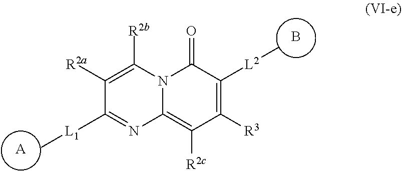
or a pharmaceutically acceptable salt, solvate, hydrate, tautomer, or stereoisomer thereof, wherein A, B, L¹, L², R^{2a}, R^{2b}, R^{2c}, R³, and subvariables thereof are as defined in claim 241;
(vi) Formula (VI-f): 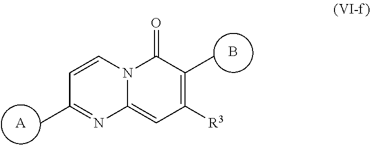
or a pharmaceutically acceptable salt, solvate, hydrate, tautomer, or stereoisomer thereof, wherein A, B, R³, and subvariables thereof are as defined in claim 241;
(vii) Formula (VI-g): 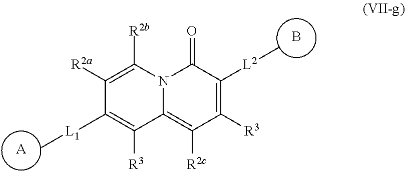
or a pharmaceutically acceptable salt, solvate, hydrate, tautomer, or stereoisomer thereof, wherein A, B, L¹, L², R^{2a}, R^{2b}, R^{2c}, R³, and subvariables thereof are as defined in claim 241; or
(viii) Formula (VI-h): 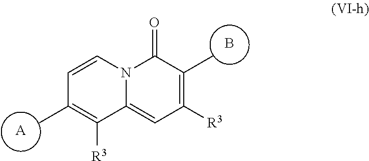
or a pharmaceutically acceptable salt, solvate, hydrate, tautomer, or stereoisomer thereof, wherein A, B, R³, and subvariables thereof are as defined in claim 241.

282-297. (canceled)

298. The compound of claim 241, wherein for Formula (VII), A is a nitrogen-containing heteroaryl and B is a nitrogen-containing heterocyclyl, wherein each heterocyclyl and heteroaryl is optionally substituted with one or more R¹.

299. (canceled)

300. The compound of claim 241, wherein for Formula (VII):
(i) one of A and B is independently selected from 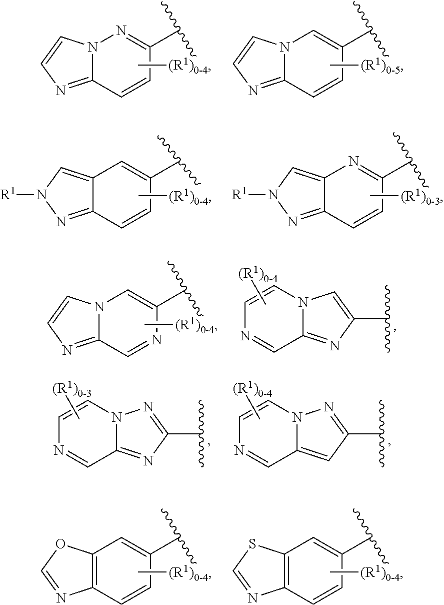
wherein R¹ is as described in claim 241; and/or
(ii) one of A and B is independently selected from 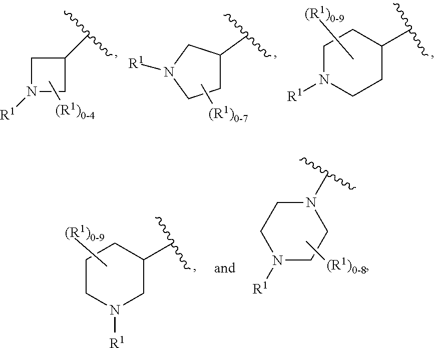
wherein R¹ is as described in claim 241.

301-323. (canceled)

324. The compound of claim 241, wherein for Formula (VII):
(i) each of L¹ and L² is independently absent; and/or
(ii) each of R^{2a}, R^{2b}, and R^{2c} is independently selected from hydrogen or alkyl (e.g., CH₃).

325-331. (canceled)

332. The compound of claim 241, wherein for Formula (VII), the compound of Formula (VII) is a compound of:
(i) Formula (VII-a): 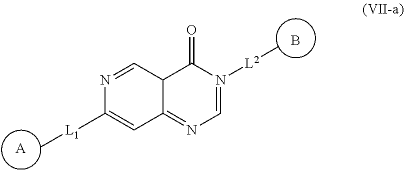
or a pharmaceutically acceptable salt, solvate, hydrate, tautomer, or stereoisomer thereof, wherein A, B, L¹, L², and subvariables thereof are as defined in claim 241; or
(ii) Formula (VII-b): 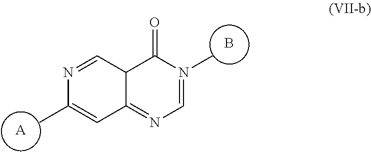
or a pharmaceutically acceptable salt, solvate, hydrate, tautomer, or stereoisomer thereof, wherein A, B, and subvariables thereof are as defined in claim 241.

333. (canceled)

334. The compound of claim 241, the compound of Formula (VI) or (VII) is a compound provided in Tables 6 or 7.

335. A method of treating a proliferative disease in a subject comprising administering to the subject a compound selected from one of Formula (VIII) or Formula (IX), wherein:
(i) the compound of Formula (VIII) is: 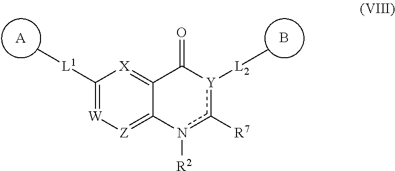
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
each of L¹ and L² is independently absent, C₁-C₆-alkylene, C₁-C₆-heteroalkylene, —O—, —C(O)—, —N(R⁸)—, —N(R⁸)C(O)—, or —C(O)N(R′)—, wherein each alkylene and heteroalkylene is optionally substituted with one or more R⁹;
each of W, X, and Z is independently C(R³) or N;
Y is N, C, or C(R^{4b}), wherein the dashed lines in the ring comprising Y may be single or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R² is absent, hydrogen, or C₁-C₆-alkyl;
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D};
R^{4b} is hydrogen, C₁-C₆-alkyl, C₁-C₆-heteroalkyl, or C₁-C₆-haloalkyl;
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶;
each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
R⁷ is hydrogen, C₁-C₆-alkyl, C₁-C₆-heteroalkyl, C₁-C₆-haloalkyl, halo, cyano, oxo, or —OR^{A};
each R⁸ is independently hydrogen, C₁-C₆-alkyl, or C₁-C₆-haloalkyl;
each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each R^{B} and R^{C} is independently hydrogen, C₁-C₆ alkyl, C₁-C₆ heteroalkyl, cycloalkyl, heterocyclyl, —OR^{A}; or
R^{B} and R^{C} together with the atom to which they are attached form a 3-7-membered heterocyclyl ring optionally substituted with one or more R¹⁰;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R¹⁰ is independently C₁-C₆-alkyl or halo; and
x is 0, 1, or 2;
or
(ii) wherein the compound of Formula (IX) is: 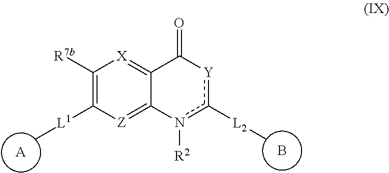
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
each of L¹ and L² is absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹;
each of X and Z is independently C(R³) or N;
Y is N, N(R^{4a}), C(R^{4b}), or C(R^{4b})(R^{4c}), wherein the dashed lines in the ring comprising Y may be single or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R² is absent, hydrogen, or C₁-C₆-alkyl;
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D};
R^{4a} is hydrogen, C₁-C₆-alkyl, C₁-C₆-heteroalkyl, or C₁-C₆-haloalkyl;
each of R^{4b} and R^{4c} is independently hydrogen, C₁-C₆-alkyl, C₁-C₆-heteroalkyl, C₁-C₆-haloalkyl, halo, or —OR^{A};
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶;
each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
R^{7b} is hydrogen, C₁-C₆-alkyl, C₁-C₆-heteroalkyl, C₁-C₆-haloalkyl, halo, cyano, or —OR^{A};
each R⁸ is independently hydrogen, C₁-C₆-alkyl, or C₁-C₆-haloalkyl;
each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each R^{B} and R^{C} is independently hydrogen, C₁-C₆ alkyl, C₁-C₆ heteroalkyl, cycloalkyl, heterocyclyl, —OR^{A}; or
R^{B} and R^{C} together with the atom to which they are attached form a 3-7-membered heterocyclyl ring optionally substituted with one or more R¹⁰;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R¹⁰ is independently C₁-C₆-alkyl or halo; and
x is 0, 1, or 2.

336. (canceled)

337. The method of claim 335, wherein for Formula (VIII) or (IX), one of A and B is independently a nitrogen-containing heterocyclyl or nitrogen-containing heterocyclyl optionally substituted with one or more R¹.

338. (canceled)

339. The method of claim 335, wherein for Formula (VIII):
(i) one of A and B is independently selected from 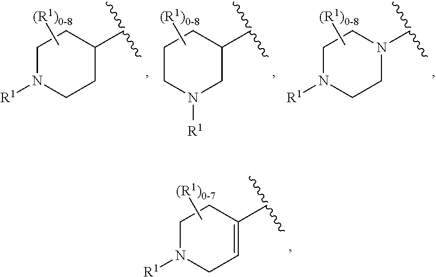
and, wherein R¹ is as described in claim 335; and/or
(ii) one of A and B is independently selected from 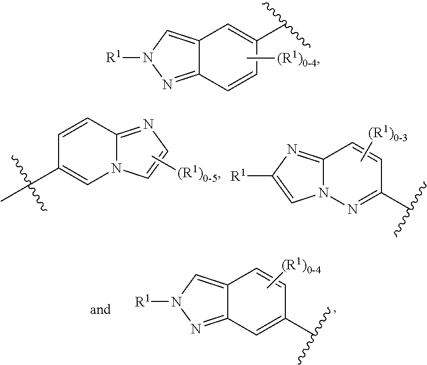
wherein R¹ is as described in claim 335.

340-363. (canceled)

364. The method of claim 335, wherein for Formula (VIII):
(i) each of L¹ and L² is independently absent;
(ii) W is C(R³)(e.g., CH);
(iii) X is C(R³)(e.g., CH);
(iv) Z is C(R³)(e.g., CH);
(v) Y is N;
(vi) R² is absent; and/or
(vii) R⁷ is hydrogen.

365-370. (canceled)

371. The method of claim 335, wherein for Formula (VIII), the compound of Formula (VIII) is a compound of:
(i) Formula (VIII-a): 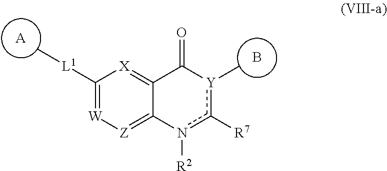
or a pharmaceutically acceptable salt, solvate, hydrate, tautomer, or stereoisomer thereof, wherein A, B, L¹, W, X, Y, Z, R⁷, and subvariables thereof are as defined in claim 335;
(ii) Formula (VIII-b): 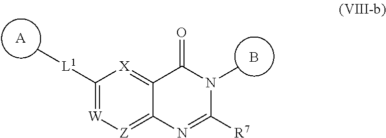
or a pharmaceutically acceptable salt, solvate, hydrate, tautomer, or stereoisomer thereof, wherein A, B, L¹, W, X, Z, R⁷, and subvariables thereof are as defined in claim 335; or
(iii) Formula (VIII-c): 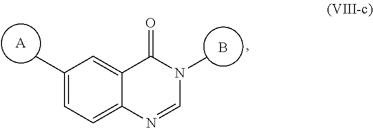
or a pharmaceutically acceptable salt, solvate, hydrate, tautomer, or stereoisomer thereof, wherein A, B, and subvariables thereof are as defined in claim 335.

372-373. (canceled)

374. The method of claim 335, wherein for Formula (VIII), the compound of Formula (VIII) is selected from a compound listed in Table 8 or a pharmaceutically acceptable salt, solvate, hydrate, tautomer, or stereoisomer thereof.

375-406. (canceled)

407. The method of claim 335, wherein for Formula (IX):
(i) each of L¹ and L² is independently absent;
(ii) W is C(R³)(e.g., CH);
(iii) Y is NH;
(iv) Z is C(R³)(e.g., CH);
(v) R² is absent; and/or
(vi) R^{7b} is hydrogen.

408-412. (canceled)

413. The method of claim 335, wherein for Formula (IX), the compound of Formula (IX) is a compound of:
(i) Formula (IX-a): 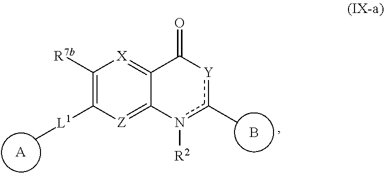
or a pharmaceutically acceptable salt, solvate, hydrate, tautomer, or stereoisomer thereof, wherein A, B, L¹, X, Y, Z, R², R^{7b}, and subvariables thereof are as defined in claim 335; or
(ii) Formula (IX-b): 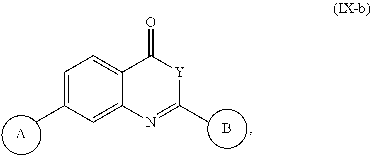
or a pharmaceutically acceptable salt, solvate, hydrate, tautomer, or stereoisomer thereof, wherein A, B, Y, and subvariables thereof are as defined in claim 335.

414. (canceled)

415. The method of claim 335, wherein for Formula (IX), the compound of Formula (IX) is a compound listed in Table 9 or a pharmaceutically acceptable salt, solvate, hydrate, tautomer, or stereoisomer thereof.

416. The method of claim 335, wherein the compound selected from one of Formula (VIII) or Formula (IX) is formulated as a pharmaceutical composition.

417-430. (canceled)

431. A pharmaceutical composition comprising a compound of claim 1 or a pharmaceutically acceptable salt, and a pharmaceutically acceptable excipient.

432. The compound of claim 1, wherein the compound:
(i) alters a target nucleic acid (e.g., an RNA, e.g., a pre-mRNA);
(ii) binds to a target nucleic acid (e.g., an RNA, e.g., a pre-mRNA); or
(iii) stabilizes a target nucleic acid (e.g., an RNA, e.g., a pre-mRNA).

433. A method of forming a complex comprising a component of a spliceosome (e.g., a major spliceosome component or a minor spliceosome component), a nucleic acid (e.g., a DNA, RNA, e.g., a pre-mRNA), and a compound of claim 1:
comprising contacting the nucleic acid (e.g., a DNA, RNA, e.g., a pre-mRNA) with a compound of Formula (I), (II), (III), (IV), (V), (VI), or (VII).

434. A method for treating a disease or disorder in a subject comprising administering to the subject a compound of claim 1 or a pharmaceutically acceptable salt or a composition thereof.

435. The method of claim 434, wherein the disease or disorder comprises:
(i) a proliferative disease (e.g., cancer, a benign neoplasm, or angiogenesis);
(ii) a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease;
(iii) neurological disease or disorder; and/or
(iv) Huntington's disease.
